(19) United States
(12) Patent Application Publication
(10) Pub. No.: US 2019/0017122 A1
(43) Pub. Date: Jan. 17, 2019

(54) MIRNAS AS DIAGNOSTIC BIOMARKERS TO DISTINGUISH BENIGN FROM MALIGNANT THYROID TUMORS

(71) Applicant: Interpace Diagnostics, LLC, Parsippany, NJ (US)

(72) Inventors: Sylvie Beaudenon-Huibregtse, Austin, TX (US); Dennis Wylie, Austin, TX (US); Alex T. Adai, Austin, TX (US)

(21) Appl. No.: 15/873,067

(22) Filed: Jan. 17, 2018

Related U.S. Application Data
(63) Continuation of application No. 13/662,450, filed on Oct. 27, 2012, now abandoned.
(60) Provisional application No. 61/552,451, filed on Oct. 27, 2011.
(60) Provisional application No. 61/552,762, filed on Oct. 28, 2011.

(51) Int. Cl. C12Q 1/6886

(52) U.S. Cl. CPC C12Q 1/6886; C12Q 2600/178; C12Q 2600/158

(57) ABSTRACT

Embodiments concern methods and compositions for distinguishing benign from malignant thyroid tumor in a patient based on the expression levels of one or more miRNAs.

The application claims priority to U.S. Provisional Patent Application 61/552,451 filed on Oct. 27, 2011 and U.S. Provisional Patent Application 61/552,762 filed on Oct. 28, 2011, both of which are hereby incorporated by reference.

### BACKGROUND OF THE INVENTION

### I. Field of the Invention

The invention relates generally to the field of molecular biology. More particularly, it concerns methods and compositions involving microRNA molecules (miRNAs). Certain aspects of the invention include applications for miRNAs in diagnostics, prognostics, and therapy for thyroid cancer.

### II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and arise from longer precursors, which are transcribed from non-protein-encoding genes. See review of Carrington et al. (2003). The precursors form structures that fold back on themselves in self-complementary regions; they are then processed by the nuclease Dicer in animals or DCL1 in plants. miRNA molecules interrupt translation through precise dr imprecise base-pairing with their targets.

Many miRNAs are conserved among diverse organisms, and this has led to the suggestion that miRNAs are involved in essential biological processes throughout the life span of an organism (Esquela-Keischer and Slack, 2006). In particular, miRNAs have been implicated in regulating cell growth and cell and tissue differentiation, cellular processes that are associated with the development of cancer. For instance, lin-4 and let-7 both regulate passage from one larval state to another during C. elegans development (Ambros, 2001). miR-14 and bantam are Drosophila miRNAs that regulate cell death, apparently by regulating the expression of genes involved in apoptosis (Brennecke et al., 2003, Xu et al., 2003).

Research on miRNAs is increasing as scientists are beginning to appreciate the broad role that these molecules play in the regulation of eukaryotic gene expression. In particular, several recent studies have shown that expression levels of numerous miRNAs are associated with various cancers (reviewed in Esquela-Kerscher and Slack, 2006). Reduced expression of two miRNAs correlates strongly with chronic lymphocytic leukemia in humans, providing a possible link between miRNAs and cancer (Calin et al., 2002). Others have evaluated the expression patterns of large numbers of miRNAs in multiple human cancers and observed differential expression of almost all miRNAs across numerous cancer types (Lu et al., 2005).

Thus, compelling evidence indicates that miRNAs play key roles in the development of many diseases and cancers, including thyroid cancer. Thyroid cancer is the fastest growing cancer diagnosis in the US with a total of 44,670 new cases and 1,690 deaths expected in 2010. From nuclear disasters such as Chemobyl in 1986, it is clear that radiation exposure is a significant risk factor for thyroid cancer but, the majority of thyroid cancers appear to be sporadic in nature. Thyroid cancers encompass a variety of lesions that range from benign adenoma to malignant tumors. They also span the spectrum from, well-differentiated, poorly differentiated or undifferentiated (anaplastic). More than 95% of thyroid cancers are derived from thyroid follicular cells, while 2-3% of thyroid tumors (medullary thyroid cancers) are derived from the calcitonin producing C-cells.

A number of genetic alterations have been shown to be involved in the development of follicular cell-derived cancers. These point mutations and translocations occur in genes for several important signaling pathways, in particular the mitogen-activated protein kinase (MAPK) pathway, and are required for transformation of well-differentiated follicular cell-derived thyroid cancers, i.e., papillary thyroid cancer (PTC) and follicular thyroid cancer (FTC).

Asuragen miRInform Thyroid Panel Assay tests for the most common gene alterations identified in follicular cell-derived thyroid carcinomas. The panel consists of 7 genes that are either mutated (BRAF, NRAS, HRAS, KRAS) or rearranged by translocation (RET/PTC1, RET/PTC3, and PAXS/PPARγ). However, approximately 30% of thyroid cancers do not carry any known mutation and therefore cannot be identified using a test based on these markers only.

Methods and compositions that improve the ability to distinguish between benign thyroid conditions from malignant thyroid tumors, including mutation- and translocation-negative tumors.

### SUMMARY OF THE INVENTION

Disclosed herein are methods and compositions for evaluating a thyroid sample from a patient to provide a clinician with information that is useful for determining diagnosis and/or treatment options. Methods involve obtaining information about the levels of expression of certain microRNAs or miRNAs whose expression levels differ between malignant and benign cells and tissue in the thyroid. In some embodiments, differences in miRNA expression between thyroid malignancies and benign thyroid cells and tissue are highlighted when expression level differences are first compared among two or more miRNAs and those differential values are compared to or contrasted with the differential values of malignant thyroid growths from non-malignant or benign thyroid growths. Such growths refer to abnormal growth of the thyroid and they include but are not limited to what are referred to as tumors, cysts, and modules.

Embodiments concern methods and compositions that can be used for evaluating a thyroid sample; categorizing a thyroid sample; evaluating a thyroid sample that has been tested for a mutation associated with malignancy; distinguishing a thyroid sample or growth; identifying a thyroid sample or growth as malignant; identifying a thyroid sample as benign; characterizing a BRAF V600E-negative thyroid sample or growth; characterizing a BRAF V600E-positive thyroid sample or growth; characterizing a thyroid sample or growth whose BRAF V600E status is not yet known; characterizing a thyroid sample or growth suspected of being malignant; characterizing a thyroid sample or growth that has already undergone a cytology or histology evaluation; testing a thyroid sample or growth known to be negative for mutations associated with thyroid malignancies; identifying thyroid tissue as a target for thyroidectomy; determining whether a. thyroid sample or growth is malignant; determining the thyroid should not be subject to a thyroidectomy; categorizing a thyroid tumor, cyst, or nodule; diagnosing a thyroid tumor, growth, or cyst; evaluating a recurrent thyroid tumor, growth or cyst; providing a prognosis to a patient regarding a thyroid tumor, growth or cyst; evaluating treatment options for a thyroid. sample suspected of being malignant; monitoring a patient's thyroid; or, treating a patient with malignant thyroid cancer. These methods can be implemented involving steps and compositions described below in different embodiments.

In some embodiments methods for evaluating a thyroid sample are provided in which methods involve measuring the level of expression of one or more miRNAs or the miRNA precursors, or one or more targets of the miRNA. In some embodiments, the thyroid sample has not been evaluated for any mutations associated with malignancy. In other embodiments, the thyroid sample has been tested for a BRAF V600E mutation. In certain cases, the thyroid sample from a patient has been determined to be negative for a BRAF V600E mutation. In further embodiments, the thyroid sample has been alternatively or additionally tested for a point mutation in one or more of the following; N-Ras, H-Ras or K-Ras and/or for the following genetic alterations: RET/PTC 1 (translocation), RET/PTC3 (translocation), and/or PAX8-PPARg Fusion Protein (PPFP) (translocaticn); these will be collectively referred to as mutations. In certain embodiments, a thyroid sample has been determined to be negative for a BRAF V600E mutation. In other embodiments, the thyroid sample has been determined to be negative for the following mutations: N-Ras, H-Ras, K-Ras, RET/PTC 1 (translocation), RET/PTC3 (translocation), PAX8-PPARg Fusion Protein (PPFP) (translocation). In further embodiments, however, a thyroid sample has been determined to be positive for a mutation related to thyroid malignancy, such as N-Ras, H-Ras, K-Ras, RET/PTC 1 (translocation), RET/PTC3 (translocation), PAX8-PPARg Fusion Protein (PPFP) (translocation). In some embodiments, a patient may be tested for a BRAF V600E mutation by having a thyroid sample assayed for miR-146b expression (Chou et al. 2010, which is hereby incorporated by reference).

In some embodiments, methods and steps discussed below are implemented on a thyroid sample that has been determined to be negative for a BRAF V600E mutation, to be positive for a mutation in N-Ras, H-Ras, K-Ras. RET/FTC 1 (translocation), RET/PTC3 (translocation), and/or PAX8-PPARg Fusion Protein (PPFP) (translocation), or to be negative for a mutation in BRAF V60E, N-Ras, H-Ras, K-Ras, RET/PTC 1 (translocation), RET/PTC3 (translocation), and PAX8-PPARg Fusion Protein (PPFP) (translocation), or to have an unknown mutation status related to BRAF V600E, N-Ras, H-Ras, K-Ras, RET/PTC 1 (translocation), RET/PTC3 (translocation), and PAX8-PPARg Fusion Protein (PPFP) (translocation). In some embodiments, methods include determining whether a thyroid sample has a BRAF V600E mutation. In even further embodiments, methods include assaying the sample for a BRAF V600E mutation. This assay may be performed before, after, or at the same time that the expression level of one or more miRNAs or other biomarkers is measured. Also, this assay may be performed before, after, or at the same time that a sample is assayed for a mutation in N-Ras, H-Ras, and/or K-Ras and/or for RET/PTC 1 (translocation), RET/PTC3 (translocation), and/or PAX8-PPARg Fusion Protein (PPFP) (translocation).

Methods for evaluating a. thyroid sample generally concern determining expression levels of one or more miRNAs because patterns of expression levels differ between malignant thyroid samples and benign thyroid samples. In some embodiments, methods are particularly valuable because they are able to distinguish malignant thyroid samples from benign thyroid samples when those samples were previously undistinguishable either from their pathology and/or from other genetic analysis. The sample may or may not have been evaluated by a cytology or histology to determine whether there were malignant cells present.

In some embodiments there are methods for evaluating a thyroid sample from a patient determined to be BRAF V600E-negative comprising a) measuring the level of expression of at least one biomarker miRNAs, wherein the biomarker is indicative of a thyroid malignancy; b) comparing the expression level of the at least one biomarker miRNA to the expression level of a comparative marker from the sample; and, c) evaluating the thyroid sample by calculating a score based on the compared expression levels, wherein the score indicates probability that the thyroid sample is benign or malignant.

Additional embodiments concern methods for determining whether a thyroid sample from a patient is malignant comprising: a) measuring expression levels of at least two differentially expressed diff pair miRNAs in the sample, wherein the expression of one miRNA is increased in a malignant thyroid sample relative to its level in a benign thyroid sample or other control and the expression of a second miRNA is decreased in malignant thyroid sample relative toils level in a benign thyroid sample or other control; b) calculating a diff pair value based on the expression levels of the increased miRNA and the decreased miRNA; and, c) evaluating the thyroid sample by calculating a score based on the compared expression levels, wherein the score indicates probability that the thyroid sample is benign or malignant.

In some embodiments there are methods for evaluating a thyroid sample from a patient comprising: a) measuring the level of expression of miR-375 or its precursor in the sample; b) comparing the expression level of miR-375 or its precursor in the sample to the level of expression of a first comparative miRNA; and, c) evaluating the sample based on the expression levels of miR-375, or its precursor and the comparative miRNA, wherein the evaluation assesses whether the sample comprises a malignant thyroid tumor. In some embodiments, methods further comprise measuring the level of expression of miR-34a-5p in the sample and comparing the miR-34a-5p expression level to the level of expression of the first or a second comparative miRNA. In additional embodiments, the first comparative miRNA is miR-7-5p. In particular embodiments, the sample's BRAF V600E mutation status has already been determined and known; in other embodiments, methods include determining whether the sample has a BRAF V600E mutation. In certain other embodiments, methods include assaying the sample for a BRAF V600E mutation.

Methods may involve measuring the level of expression of a biomarker miRNA. The term “biomarker miRNA” refers to a miRNA whose expression level is indicative of a particular disease or condition. In some embodiments the biomarker miRNA is one whose expression level is detectably increased in one population relative to another population, such as in malignancies compared to a benign thyroid growth. In other embodiments, a biomarker miRNA is one whose expression level is detectably decreased in one population relative to another population. A biomarker miRNA may be a diff pair miRNA. In certain embodiments. As part of a diff pair, the level of expression of a biomarker miRNA may highlight or emphasize differences in miRNA expression between different populations, such as a thyroid malignancy and a thyroid growth that is benign. In some embodiments, when miRNA expression is different in a particular population relative to another population, differences between miRNA expression levels can be increased, highlighted, emphasized, or otherwise more readily observed in the context of a diff pair. In some embodiments a biomarker miRNA is miR-10b, miR-15a, miR-21, miR-24-1-star, miR-24-2-star, miR-27a, miR-27b-star, miR-31, miR-31-star, miR-34a, miR-34a-star, miR-34b-star, miR-127-3p, miR-132, miR-132-star, miR-138-1-star, miR-140-5p, miR-142-5p, miR-146a, miR-146b-3p, miR-146b-5p, miR-155, miR-181a-star, miR-181c, miR-182, miR-196a, miR-205, miR-221, miR-221-star, miR-222, miR-222-star, miR-224, miR-301a, miR-329, miR-335, miR-340, miR-346, miR-369-5p, miR-375, miR-376c, miR-409-3p, milt-425, miR-493-star, miR-495, miR-539, miR-551b, miR-1827, miR-1910, miR-3065-5p, miR-3200-3p, and/or miR-4288. In further embodiments, a biomarker miRNA is measuring the level of expression of at least two of the following biomarker miRNAs: miR-375, miR-146b-3p, miR-146b-5p, miR-221-3p, miR-222-3p, miR-551b-3p, miR-204-5p, miR-7-5p, miR-141-3p, miR-31-5p, miR-81a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, and/or miR-182-5p. In certain embodiments, the first 3, 4, 5, 6, 7, 8, 9, 10, 11, 12, 13, 14, 15, 16, 17, 18, 19, 20, 21, 22, 23, 24, 25 or more (or any range derivable therein) or more of the miRNAs listed in the previous paragraph may be used in certain embodiments. In some embodiments a biomarker miRNA is one or more of the following: miR-10b, miR-15a, miR-21, miR-24-1-5p, miR-24-2-5p, miR-27a, miR-27b-5p, miR-31-5p, miR-31-3p, miR-34a-5p, miR-34a-3p, miR-34b-5p, miR-127-3p, miR-132-3p, miR-132-5p, miR-138-1-3p, miR-140-5p, miR-142-5p, miR-146a-5p, miR-146b-3p, miR-146b-5p, miR-155-5p, miR-181a-2-3p, miR-181c-5p, miR-182-5p, miR-196a-5p, miR-205-5p, miR-221-3p, miR-221-5p, miR-222-3p, miR-222-5p, miR-224-5p, miR-301a-3p, miR-329, miR-335-5p, miR-340-5p, miR-346, miR-369-5p, miR-375, miR-376c-3p, miR-409-3p, miR-425, miR-493-5p, miR-495-3p, miR-539-5p, miR-551b-3p, miR-1827, miR-1910, miR-3065-5p, miR-3200-3p, and/or miR-4288.

It is contemplated that in some embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38,39, 40, 41, 42, 43, 44, 45, 46, 47, 48, 49, or 50 biomarker miRNAs (or any range derivable therein) may be measured. In some embodiments, a biomarker miRNA, is an miRNA listed in Table 1, 4D, or 5B. In other embodiments at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biomarker miRNAs are measured. In certain embodiments, any miRNA shown in Table 4A or 4B may be a biomarker or comparative miRNA. In further embodiments it is contemplated that a method or other embodiments may include at least or at most embodiments 1, 2, 3, 4, 5, 6, 7, 8, 9, 10; 11, 12, 13, 14, 15, 16, 17, 18, 19, 20, 21, 22, 23, 24, 25, 26, 27, 28, 29. or 30 (or any range derivable therein) of the following miRNAs: miR-375, miR-146b-3p, miR-46b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, and/or miR-182-5p. In specific embodiments, 1, 2, 3, 4, 5, 6, 7, 8, 9, 10, 11, or all 12 of the following miRNAs are included: miR-375, miR-146b-3p, miR-146b-5p, miR-221-3p; miR-222-3p, miR-551b-3p, miR-204-5p, miR-7-5p, miR-141-3p, miR-31-5p, miR-181a-2-3p, and miR-15a-5p. In further embodiments, 1, 2, 3, 4, 5, 6, 7, 8, or all 9 (any any range derivable therein) of the following miRNAs are included additional or alternatively to the previous set: miR-224-5p, miR-221-5p, miR-222-5p, miR-21-5p, miR-424-5p, miR-34a-5p, miR-197-3p, miR-19b-3p, miR-155-5p. In additional embodiments, 1, 2, 3, 4, 5, 6, 7, 8, 9, 11, or 12 (or any range derivable therein) of the following miRNAs may be included: miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, and miR-182-5p. In some cases, miR-224-5p and/or miR-222-5p may be included.

Measuring a microRNA or miRNA refers to measuring the amount of a mature microRNA or miRNA, though it is contemplated that a mature miRNA may be indirectly determined by measuring the level of an immature or unprocessed form of the miRNA, such as the double-stranded RNA molecule or RNA hairpin structure. Moreover, in some embodiments, the amount of a mature miRNA is determined by measuring the amount of one or more of the miRNA's target or the targets complement. An miRNA's target refers to the endogenous RNA in the thyroid cell that is the target for the miRNA and whose expression is affected by the miRNA. Consequently, any embodiments discussed herein in the context of determining the amount of a microRNA (i.e., the mature form of a microRNA) can be implemented instead by measuring a precursor of the miRNA or one or more of the miRNA's target (or the complement thereof). Unless qualified, the term “measuring” refers to directly measuring. Mature miRNAs may be indirectly determined by directly measuring precursor microRNA molecules. It will be understood that the term “star” in the context of a miR refers to an asterisk (*); for example, miR-222-star is the same as miR-222*.

Measuring or assaying for expression levels of a microRNA can be accomplished by a variety of different chemical and/or enzymatic reactions that are well known to those of skill in the art. In certain embodiments, methods may involve amplification and/or hybridization. It is contemplated that the level of a mature microRNA (miRNA) may be indirectly determined by measuring the level of the immature or unprocessed microRNA. Whether the mature or immature form of a microRNA is measured depends on the detection method, such as which primer or probe is used in the method. A person of ordinary skill in the art knows how this would be implemented.

Some methods also involve comparing the expression level of the at least one biomarker miRNA to the expression level of a comparative marker from the sample. In other embodiments, methods involve comparing the expression level of at least one biomarker miRNA to the expression level of that biomarker miRNA in a standardized sample, such as a sample known to be thyroid malignancy or known to be a benign thyroid condition. An increase or decrease in the level of expression will be evaluated. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative markers (or any range derivable therein) may be used in comparisons or compared to the expression level of a biomarker or of a biomarker miRNA. In other embodiments at least or at most 1, 2, 3, 4, 5, 6, 7, 8, 9, 10, 11, 12, 13, 0.14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative markers are measured. In particular embodiments, at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative markers are compared to one or more biomarker miRNAs. A “comparative marker” refers to a gene product (such as a protein, RNA transcript, miRNA, or unprocessed miRNA) whose expression level is used to evaluate the level of an miRNA in the sample; in some embodiments, the expression level of a comparative miRNA is used to evaluate a biomarker miRNA expression level. In specific embodiments, the comparative marker is an miRNA. Moreover, in certain embodiments, a comparative marker may also be a biomarker miRNA. In particular cases, a comparative marker is one whose expression level appears to change in the opposite direction as a biomarker miRNA against which it is compared. For example, a biomarker may have an increased expression level in a malignant thyroid sample as compared to a benign thyroid sample. The comparative marker that is used to compare against that biomarker may have a decreased expression level in a malignant thyroid sample as compared to a benign thyroid sample.

In some embodiments, the comparative marker is miR-7, miR-139-5p, miR-185-star, miR-486-5p, miR-519a, miR-1910, or miR-3123. In additional embodiments, a comparative miRNA is an miRNA listed in Table 1, 4D, or 5B. In further embodiments, a comparative marker is any of miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, and/or miR-182-5p.

Some methods involve measuring expression levels of at least two differentially expressed diff pair miRNAs in the sample, wherein the expression of one miRNA is increased in a malignant thyroid sample relative to a benign thyroid sample and the expression of a second miRNA is decreased in malignant thyroid sample relative to a benign thyroid sample. In some embodiments, the increased miRNA is miR-10b, miR-15a, miR-21, miR-24-1-star, miR-24-2-star, miR-27a, miR-27b-star, miR-31, miR-31-star, miR-34a, miR-34a-star, miR-34b-star, miR-127-3p, miR-132, miR-132-star, miR-138-1-star, miR-140-5p, miR-142-5p, miR-146a, miR-146b-3p, miR-146b-5p, miR-155, miR-181a-star, miR-181c, miR-182, miR-196a, miR-205, miR-221, miR-221-star, miR-222, miR-222-star, miR-224, miR-301a, miR-329, miR-335, miR-340, miR-346, miR-369-5p, miR-375, miR-376c, miR-409-3p, miR-425, miR-493-star, miR-495, miR-539, miR-551b, miR-1827, miR-1910, miR-3065-5p, miR-3200-3p, or miR-4288. It is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of these increased miRNAs (or any range derivable therein) may be measured. In some embodiments, an increased miRNA is an miRNA listed in Tables 1, 4D, or 5B that shows a relative increase in expression. The term “diff pair miRNA” refers to a miRNA that is one member of a pair of miRNAs where the expression level of one miRNA of the diff pair in a sample is compared to the expression level of the other miRNA of the diff pair in the same sample. The expression levels of two diff pair miRNAs may be evaluated with respect to each other, i.e., compared, which includes but is not limited to subtracting, dividing, multiplying or adding values representing the expression levels of the two diff pair miRNAs. A. “diff pair” refers to the pair of diff pair miRNAs (or other marker). The diff pair is identified by an entity in front of a slash (/) and an entity following the slash. In embodiments described herein, the biomarker miRNA is understood as the entity in front of the slash and the comparative marker follows the slash. Moreover, it is readily apparent that the miRNA used as a biomarker and the miRNA used as the comparative miRNA may be switched, and that any calculated value can be evaluated accordingly by a person of ordinary skill in the art. However, a person of ordinary skill in the art understands that different pair analysis may be adjusted, particular with respect to altering the comparative miRNA in a pair without affecting the concept of the embodiments discussed herein.

In some embodiments it is specifically contemplated that the expression level of miR-467b in the sample is not measured. In other embodiments, the expression level of miR-221 and/or miR-222 in the sample is not measured. In further embodiments, they may be measured, but not to determine whether a sample is benign or malignant but for subclassifying a sample. In some embodiments, methods involve determining at least one diff pair value based on the expression levels of a diff pair, wherein the biomarker miRNA and a comparative marker are the diff pair. A differential pair value or diff pair value between the biomarker miRNA and the comparative marker can be calculated or determined or evaluated; this value is a number that is referred to as a “diff pair value” when it is based on the expression level of two miRNAs (or their precursors or targets). A diff pair value can be calculated, determined or evaluated using one or more mathematical formulas or algorithms. In some embodiments, the value is calculated, determined or evaluated using computer software. In particular embodiments, the diff pair is milt-375/miR-7. In other embodiments, the diff pair is miR-34a/miR-7 (miR-7 is synonymous with miR-7-5p). In additional embodiments a diff pair used in embodiments is 1, 2, 3, 4, 5, 6, 7, 8, or all 9 (and any range derivable therein) of the following diff pairs: miR-375/miR-7-5p, miR-375/miR-204-5p, miR-375/miR-141-3p, miR-146b-5p/miR-7-5p, miR-146b-5p/miR-204-5p, miR-146b-5p/miR-141-3p, miR-146b-3p/miR-204-5p, miR-146b-3p/mir-7, and/or miR-146b-3p/miR-141-3p.

In some embodiments, a weighted coefficient may be applied to a diff pair value. In certain embodiments, a diff pair value for miR-375/miR-7-5p is weighted more heavily than a diff pair value for miR-34a/miR-7-5p. In other embodiments, a diff pair value for miR-34a/miR-7 is weighted more heavily than the diff pair value for miR-375/miR-7-5p. In some embodiments, the diff pair value for miR-375/miR-7-5p is weighted more heavily than a diff pair value for 1, 2, 3, 4, 5, 6, 7, or all 8 of miR-375/miR-204-5p, miR-375/miR-141-3p, miR-146b-5p/miR-7-5p, miR-146b-5p/miR-204-5p, miR-146b-5p/miR-141-3p, miR-146b-3p/miR-204-5p, miR-146b-3p/miR-7-5p, and/or miR-146b-3p/miR-141-3p, miR-375/miR-7-5p, miR-375/miR-204-5p, miR-375/miR-141-3p, miR-146b-5p/miR-7-5p, miR-146b-5p/miR-204-5p, miR-146b-5p/miR-141-3p, miR-146b-3p/miR-204-5p, miR-146b-3p/miR-7-5p, and/or miR-146b-3p/miR-141-3p. In some embodiments, the diff pair value for miR-375/miR-204-5p is weighted more heavily than a diff pair value for 1, 2, 3, 4, 5, 6, 7, or all 8 of miR-375/miR-7-5p, miR-375/miR-141-3p, miR-146b-5p/miR-7-5p, miR-146b-5p/miR-204-5p, miR-146b-5p/miR-141-3p, miR-146b-3p/miR-204-5p, miR-146b-3p/miR-7-5p, and/or miR-146b-3p/miR-141-3p. In some embodiments, the diff pair value for miR-375/miR-141-3p is weighted more heavily than a diff pair value for 1, 2, 3, 4, 5, 6, 7, or all 8 of miR-375/miR-7-5p, miR-375/miR-204-5p, miR-146b-5p/miR-7-5p, miR-146b-5p/miR-204-5p, miR-146b-5p/miR-141-3p, miR-146b-3p/miR-204-5p, miR-146b-3p/miR-7-5p, and/or miR-146b-3p/miR-141-3p. In some embodiments, the diff pair value for miR-146b-5p/miR-7-5p is weighted more heavily than a diff pair value for 1, 2, 3, 4, 5, 6, 7, or all 8 of miR-375/miR-7-5p, miR-375/miR-204-5p, miR-375/miR-141-3p, miR-146b-5p/miR-204-5p, miR-146b-5p/miR-141-3p, miR-146b-3p/miR-204-5p, miR-146b-3p/miR-7-5p, and/or miR-146b-3p/miR-141-3p. In some embodiments, the diff pair value for miR-miR-146b-5p/miR-204-5p is weighted more heavily than a diff pair value for 1, 2, 3, 4, 5, 6, 7, or all 8 of miR-375/miR-7-5p, miR-375/miR-204-5p, miR-375/miR-141-3p, miR-146b-5p/miR-7-5p, miR-146b-5p/miR-141-3p, miR-146b-3p/miR-204-5p, miR-146b-3p/miR-7-5p, and/or miR-146b-3p/miR-141-3p, miR-375/miR-7-5p, miR-375/miR-204-5p, miR-375/miR-141-3p, miR-146b-5p/miR-7-5p, miR-146b-5p/miR-204-5p, miR-146b-5p/miR-141-3p, miR-146b-3p/miR-204-5p, miR-146b-3p/miR-7-5p, and/or miR-146b-3p/miR-141-3p. In some embodiments, the diff pair value for miR-146b-3p/miR-7-5p is weighted more heavily than a diff pair value for 1, 2, 3, 4, 5, 6, 7, or all 8 of miR-375/miR-7-5p, miR-375/miR-204-5p, miR-375/miR-141-3, miR-146b-5p/miR-7-5p, miR-146b-5p/miR-204-5p, miR-146b-5p, miR-141-3p, miR-146b-3p/miR-204-5p, and/or miR-146b-3p/miR-141-3p. In some embodiments, the diff pair value for miR-146b-3p/miR-141-3p is weighted more heavily than a diff pair value for 1, 2, 3, 4, 5, 6, 7, or all 8 of miR-375/miR-7-5p, miR-375/miR-204-5p, miR-375/miR-141-3p, miR-146b-5p/miR-7-5p, miR-146b-5p/miR-204-5p, miR-146b-5p/miR-141-3p, miR-146b-3p/miR-204-5p, and/or miR-146b-3p/miR-7-5p.

Methods also include evaluating the thyroid sample by calculating a score based on the compared expression levels or diff pair values, wherein the score indicates probability that the thyroid sample is benign or malignant.

In certain embodiments, methods may involve classifying a sample as a hyperplastic nodule, follicular adenoma, oncocytic follicular adenoma, multi nodular goiter or Hashimoto's thyroiditis. In some cases, the sample is classified as a papillary carcinoma, follicular variant of papillary carcinoma, or a follicular carcinoma. The sample may be generally categorized as benign or malignant and subcategorized as one of the classifications discussed above. In certain embodiments, the categorization as benign or malignant occurs prior to subcategorization as one of the classifications, however, the testing for these may be done at the same (parallel) or different (serial) times. In some embodiments, miR-224-5p and/or miR-155-5p may be used for identifying Hashimoto's thyroiditis.

In other embodiments, a diff pair need not be evaluated and instead, a coefficient value is applied to each miRNA expression level. The coefficient value reflects the weight that the expression level of that particular miRNA has in assessing the chances that a particular pancreatic cyst is a high risk lesion or a low risk lesion. In some embodiments, instead of a diff pair value, a coefficient value is used for the measured expression level of miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-L-3p, miR-139-5p, miR-151a-3p, miR-29b-3p, miR-96-5p, miR-34a-star, miR-3065-5p, miR-34b-star, and/or miR-182. In certain embodiments, the coefficient values for a plurality of miRNAs whose expression levels are measured add up to zero (0). The plurality may be, be at least, or be at most 1, 2, 3, 4, 5, 6, 7, 8, 9, 10, 11, 12, 13, 14, 15, 16, 17, 18, 19, 20, 21, 22, 23, 24, 25, 26 of these miRNAs, as well as any miRNAs discussed herein. Methods and computer readable medium can be implemented with coefficient values instead of or in addition to diff pair values. In some cases, a coefficient may be multiplied against a diff pair value to reflect the weight of that diff pair in any analysis or diagnostic score calculation.

In some embodiments, the coefficient for miR-375 is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-146b-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-146b-5p is greater than or less than the coefficient for 1, 2, 10, 11, 12, 13, 14, 15, 16, 17, 18, 19, 20, 21, 22, 23, 24, or all 25 (any any range derivable therein) of the following miRNAs: miR-375, miR-146b-3p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-221-3p is greater than or less than. the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-222-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-551b-3p, miR-204-5p, miR-7-5p, miR-141-3p, miR-31-5p, miR-18.1a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for mil-551b-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-204-5p, miR-7-5p, miR-141-3p, miR-31-5p, miR-81a-2-3p, miR-15a-5p, miR-224-5p, miR-221-5p, miR-222-5p, miR-21-5p, miR-424-5p, miR-34a-5p, miR-197-3p, miR-19b-3p, miR-155-5p miR-138-3p, miR-139-5p, miR-151a-3p, miR-29b-3p, miR-96-5p, miR-34a-star, miR-3065-5p, miR-34b-star, and miR-182. In other embodiments, the coefficient for miR-204-5p is greater than or less than the coefficient for 1, 2, 3, 4, 5, 6, 7, 8, 9, 10, 11, 12, 13, 14, 15, 16, 17, 18, 0.19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-7-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-141-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following mil RNAs: miR-375, miR-146b-3p, miR-146b-5p, miR-221-3p, miR-222-3p, miR-551b-3p, miR-204-5p, miR-7-5p, mil-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments; the coefficient for miR-31-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any tango derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-22-3p is greater than or loss than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-38-1-3p miR-138-3p, miR-39-5p, miR-151a-3p, miR-29b-3p, miR-96-5p, miR-34a-star, miR-3065-5p, miR-34b-star, and miR-182. In other embodiments, the coefficient for miR-222-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-551b-3p, miR-204-5p, miR-7-5p, miR-141-3p, miR-31-5p, miR-181a-2-3p, miR-15a-5p, miR-224-5p, miR-221-5p, miR-222-3p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-21-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-424-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-34a-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, mil-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-197-3p, miR-19b-3p, miR-155-5p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-197-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-55.1 b-3p, miR-204-5p, miR-7-5p, miR-141-3p, miR-31-5p, miR-81a-2-3p, miR-15a-5p, miR-224-5p, miR-221-5p, miR-222-5p, miR-21-5p, miR-424-5p, miR-34a-5p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-19b-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p; miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-155-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-9b-3p, miR-138-1-3p, miR-139-5p, miR-151a-3p, miR-29b-3p, miR-96-5p, miR-34a-star, miR-3065-5p, miR-34b-star, and/or miR-182: In other embodiments, the coefficient for miR-138-1-3p is greater than or less than the coefficient for 1, 2, 3, 4, 5, 6, 7, 8, 9, 10, 11, 12, 13, 14, 15, 16, 17, 18, 19,20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-139-5p, miR-151a-3p, miR-29b-3p, miR-96-5p, miR-34a-star, miR-3065-5p, miR-34b-star, and miR-182. In other embodiments, the coefficient for miR-139-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-151a-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-29b-3p, miR-96-5p, miR-34a-star, miR-3065-5p, miR-34b-star, and miR-182. In other embodiments, the coefficient for miR-29b-3p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, mil-551b-3p, miR-204-5p, miR-7-5p, miR-141-3p, miR-31-5p, miR-181a-2-3p, miR-15a-5p, miR-224-5p, miR-221-5p; miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, miR-34b-star, and miR-182. In other embodiments, the coefficient for miR-96-5p is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-155a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-34a-star, miR-3065-5p, miR-34b-star, and miR-182. In other embodiments, the coefficient for miR-34a-star is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-3065-5p, miR-34b-star, and miR-182. In other embodiments, the coefficient for miR-3065-5p is greater than or less than the coefficient for 1,2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, mil-96-5p, miR-34a-star, miR-34b-star, and miR-182. In other embodiments, the coefficient for miR-34b-star is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-3.1-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, and miR-182. In other embodiments, the coefficient for miR-182 is greater than or less than the coefficient for 1, 2, 3, 4, 5, 6, 7, 8, 9, 10, 11, 12, 13, 14, 15, 16, 17, 18, 19, 20, 21, 22, 23, 24, or all 25 (any any range derivable therein) of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-151a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-star, miR-3065-5p, and miR-34b-star.

A difference between or among weighted coefficients may be, be at least or be at most 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 3.5, 4.0, 4.5, 5.0, 5.5, 6,0, 6,5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 times or -fold (or any range derivable therein). Moreover, a person of ordinary skill in the art would understand that the coefficients could be negative or positive (in order to make their sum be zero, for example), but that the coefficient still reflected a difference in weight that is shown above.

In some embodiments, the accuracy of methods to determine whether a thyroid sample is benign or malignant or provide a diagnostic score is, is at least, or is at most 50, 55, 60, 65, 70, 75, 80, 85, 90, 95, 96, 97, 98, 99 or 100% (and any range derivable therein). In other embodiments, the accuracy of categorizing the sample. Into a subtype of benign or malignant conditions is, is at least, or is at most 50, 55, 60, 65, 70, 75, 80, 85, 90, 95, 96, 97, 98, 99 or 100% (and any range derivable therein).

Methods for treating a patient with thyroid cancer are also provided. In some embodiments such methods comprise; a) obtaining a diagnostic score based on expression levels of miRNAs in a thyroid sample from the patient, wherein the expression levels differ between malignant thyroid cancer cells compared to benign thyroid cells and wherein the thyroid sample has been determined to be negative for a BRAF V600E mutation; and, b) performing a thyroidectomy on a patient determined to have a diagnostic score indicative of malignant thyroid cancer.

In some embodiments, a patient is also administered radioactive iodine, radiation and/or chemotherapy as part of a treatment regimen. In further embodiments, methods may involve determining the patient as having or likely having a benign thyroid condition, or determining the patient as not having or likely not having a malignancy. In such cases, a clinician may then decide not to subject the patient to surgery. In such cases, the patient may continue to be monitored. In certain embodiments, methods involve imaging an unresected thyroid growth or doing a biopsy before and/or after miRNA levels are measured. In further embodiments, the imaging or biopsy occurs after 1, 2, 3, 4, 5, 6 months following a test that involves measuring one or more miRNA expression levels.

In some embodiments, methods will involve determining or calculating a diagnostic score based on data concerning the expression level of one or more miRNAs, meaning that the expression level of the one or more miRNAs is at least one of the factors on which the score is based. A diagnostic score will provide information about the biological sample, such as, the general probability that the thyroid sample is malignant and/or an aggressive tumor or that the thyroid sample is benign. In some embodiments, the diagnostic score represents the probability that the thyroid sample is more likely than not either malignant or benign. In certain embodiments, a probability value is expressed as a numerical integer or number that represents a probability of 0% likelihood to 100% likelihood that a patient has a thyroid malignancy or a benign thyroid condition. In some embodiments, the probability value is expressed as a numerical integer or number that represents a probability of 0, 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61,62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has a particular type of thyroid condition or growth. Alternatively, the probability may be expressed generally in percentiles, quartiles, or deciles.

In some embodiments, methods include evaluating one or more differential pair values using a scoring algorithm to generate a diagnostic score for the thyroid growth in terms of being malignant or being benign, wherein the patient is identified as having or as not having such a growth based on the score. It is understood by those of skill in the art that the score is a predictive value about the classification of the thyroid growth. In some embodiments, a report is generated and/or provided that identifies the diagnostic score or the values that factor into such a score. In some embodiments, a cut-off score is employed to characterize a sample as likely having a thyroid malignancy (or alternatively a benign thyroid condition). In some embodiments, the risk score for the patient is compared to a cut-off score to characterize the biological sample from the patient with respect to a malignancy or a benign condition. In certain embodiments, the diagnostic score is calculated using a weighted coefficient for each of the measured miRNA levels of expression. The weighted coefficients will typically reflect the significance of the expression level of a particular miRNA for determining risk of a certain disease state, such as malignancy or benign status, or any one of hyperplastic nodule, follicular adenoma, oncocytic follicular adenoma, multi nodular goiter, Hashimoto's thyroiditis, papillary carcinoma, follicular variant of papillary carcinoma, or a follicular carcinoma.

In some embodiments, determination of calculation of a diagnostic score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0:058, 0.059, 0.060, 0.061, 0.062, 0.063, 0.064, 0.065, 0.066, 0.067, 0.068, 0.069, 0.070, 0.071, 0:072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain embodiments, the diagnostic score is calculated using one or more statistically significantly differentially expressed biomarkers (either individually or as diff pairs).

Methods may involve obtaining from the patient a thyroid sample, which means the sample is obtained directly from the patient. In other embodiments, a patient's thyroid tissue sample may be obtained from an entity that is not the patient, such as the doctor, clinician, hospital or laboratory. In certain embodiments, methods involve a thyroid tissue sample or a thyroid cyst or nodule sample. In particular embodiments, the sample is a tissue sample, while in other embodiments, the sample is a cystic fluid sample. In some cases, methods involve fixing the tissue sample in formalin and embedding it in paraffin prior to measuring the level of expression of one or more miRNAs or diff pair miRNAs in the sample. In additional embodiments, the sample is obtained by fine needle aspirate or FNA. In other embodiments, the sample is retrieved from a biopsy, such as a fine needle aspiration biopsy (FNAB) or a needle aspiration biopsy (NAB).

In some embodiments, a patient is determined to have a thyroid sample indicative of a malignancy or aggressive cancer. The term “indicative of a malignancy” means the data indicate that the patient likely has a malignancy, where “likely” means “greater than not.” In other embodiments, the patient is determined to have a thyroid sample indicative of a benign condition. The determination may or may not be based on a diagnostic score that is calculated based on one or more miRNA expression levels or diff pair values. In additional embodiments, methods involve determining a treatment for the patient based on one or more diff pair values. In some embodiments, methods include determining a treatment for the patient based on a calculated diagnostic score. In some embodiments, a patient may be suspected of having a thyroid malignancy. In other embodiments, the patient may have previously had a thyroid condition suspected of being malignant that was then subsequently treated. In. other embodiments, the patient has recurring thyroid growth, which may or may not be malignant. In still further embodiments, the patient has a familial history of thyroid growths, particularly malignant thyroid growths. In some circumstances, a patient also presents with symptoms of a thyroid growth, such as fatigue, change in appetite and other symptoms relating to a change in thyroid hormone levels.

Some embodiments further involve isolating ribonucleic or RNA from a biological sample. Other steps may or may not include amplifying a nucleic acid in a sample and/or hybridizing one or more probes to an amplified or non-amplified nucleic acid. In certain embodiments, a microarray may be used to measure or assay the level of miRNA expression in a sample.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs that are related to the disclosed methods and compositions, as well as their sequences, are provided herein. The name of the miRNAs that are used in methods and compositions refers to an miRNA that is at least 90% identical to the named miRNA based on its mature sequence listed herein and that is capable of being detected under the conditions described herein using the designated ABI part number for the probe. In most embodiments, the sequence provided herein is the sequence that is being measured in methods described herein. In some methods, a step may involve using a nucleic acid with the sequence comprising or consisting of any of the complements of any of SEQ ID NOs:1-65 or any sequence found herein to measure expression of a miRNA in the sample. Alternatively, probes directed to the immature form of these miRNAs may be used, as may be probes directed to the targets of the miRNAs. In some embodiments, a complement of SEQ ID NO:1 (5′-GGAGAAAUUAUCCUUGGUGUGU′3′) is used to measure expression of naturally occurring miR-539 in a sample. In some embodiments, a complement of SEQ ID NO:2 (5′-UGGAAGACUAGUGAUUUUGUUGU′3′) is used to measure expression of naturally occurring miR-7-5p in a sample. In some embodiments, a complement of SEQ ID NO:3 (5′-UGAGAACUGAAUUCCAUAGGCU′3′) is used to measure expression of naturally occurring miR-146b-5p in a sample. In some embodiments, a complement of SEQ ID NO:4 (5′-UGCCCUGUGGACUCAGUUCUGG′3′) is used to measure expression of naturally occurring miR-146b-3p in a sample. In some embodiments, a complement of SEQ ID NO:5 (5′-AACACACCUGGUUAACCUCUUU′3′) is used to measure expression of naturally occurring miR-329 in a sample. In some embodiments, a complement of SEQ ID NO:6 (5′-UCGGAUCCGUCUGAGCUUGGCU′3′) is used to measure expression of naturally occurring miR-127-3p in a sample. In some embodiments, a complement of SEQ ID NO:7 (5′-ACCUGGCAUACAAUGUAGAUUU′3′) is used to measure expression of naturally occurring miR-221* in a sample. In some embodiments, a complement of SEQ ID NO:8 (5′-CCAGUCCUGUGCCUGCCGCCU′3′) is used to measure expression of naturally occurring miR-1910 in a sample. In some embodiments, a complement of SEQ ID NO:9 (5′-AAACAAACAUGGUGCACUUCUU′3′) is used to measure expression of naturally occurring miR-495 in a sample. In some embodiments, a complement of SEQ 1D NO:10 (5′-GAAUGUUGCUCGGUGAACCCCU′3′) is used to measure expression of naturally occurring miR-409-3p in a sample. In some embodiments, a complement of SEQ ID NO:11 (5′-AACAUAGAGGAAAUUCCACGU′3′) is used to measure expression of naturally occurring miR-376c in a sample. In some embodiments, a complement of SEQ ID NO:12 (5′-UCCUUCAUUCCACCGGAGUCUG′3′) is used to measure expression of naturally occurring miR-205 in a sample. In some embodiments, a complement of SEQ ID NO:13 (5′-UUUGUUCGUUCGGCUCGCGUGA′3′) is used to measure expression of naturally occurring miR-375 in a sample. In some embodiments, a complement of SEQ ID NO:14 (5′-CUCAGUAGCCAGUGUAGAUCCU′3′) is used to measure expression of naturally occurring miR-222* in a sample. In some embodiments, a complement of SEQ ID NO:15 (5′-AGAUCGACCGUGUUAUAUUCGC′3′) is used to measure expression of naturally occurring miR-369-5p in a sample. In some embodiments, a complement of SEQ ID NO:16 (5′-UGUCUGCCCGCAUGCCUGCCUCU′3′) is used to measure expression of naturally occurring miR-346 in a sample. In some embodiments, a complement of SEQ ID NO:17 (5′-AGCUACAUUGUCUGCUGGGUUUC′3′) is used to measure expression of naturally occurring miR-221 in a sample. In some embodiments, a complement of SEQ ID NO:18 (5′-AGGCAAGAUGCUGGCAUAGCU′3′) is used to measure expression of naturally occurring miR-31 in a sample. In some embodiments, a complement of SEQ ID NO:19 (5′-UUGUACAUGGUAGGCUUUCAUU′3′) is used to measure expression of naturally occurring miR-493* in a sample. In some embodiments, a complement of SEQ ID NO:21 (5′-CACCUUGCGCUACUCAGGUCUG′3′) is used to measure expression of naturally occurring miR-3200-3p in a sample. In some embodiments, a complement of SEQ ID NO:22 (5′-UAGCUUAUCAGACUGAUGUUGA′3′) is used to measure expression of naturally occurring miR-21 in a sample. In some embodiments, a complement of SEQ ID NO:23 (5′-UGGCAGUGUCUUAGCUGGUUGU′3′) is used to measure expression of naturally occurring miR-34a in a sample. In some embodiments, a complement of SEQ ID NO:24 (5′-GCUACUUCACAACACCAGGGCC′3′) is used to measure expression of naturally occurring miR-138-1* in a sample. In some embodiments, a complement of SEQ ID NO:25 (5′-UCUACAUACACUCACUGUCUCCAG′3′) is used to measure expression of naturally occurring miR-139-5p in a sample. In some embodiments, a complement of SEQ ID NO:26 (5′-UAACCACUGUCUGGUAAAGAUGG′3′) is used to measure expression of naturally occurring miR-141-3p in a sample. In some embodiments, a complement of SEQ ID NO:28 (5′-UUAAUGCUAAUCGUGAUAGGGGU′3′) is used to measure expression of naturally occurring miR-155 in a sample. In some embodiments, a complement of SEQ ID NO:30 (5′-UUCACCACCUUCUCCACCCAGC′3′) is used to measure expression of naturally occurring miR-197-3p in a sample. In some embodiments, a complement of SEQ ID NO:31 (5′-AGCUACAUCUGGCUACUGGGU′3′) is used to measure expression of naturally occurring miR-222 in a sample. In some embodiments, a complement of SEQ ID NO:32 (5′-CAAGUCACUAGUGGUUCCGUU′3′) is used to measure expression of naturally occurring miR-224 in a sample. In some embodiments, a complement of SEQ ID NO:33 (5′-AAUGACACGAUCACUCCCGUUGA′3′) is used to measure expression of naturally occurring miR-425 in a sample. In some embodiments, a complement of SEQ ID NO:34 (5′-UJAGAGAACUGAAUUCCAUGGGUU′3′) is used to measure expression of naturally occurring miR-146a in a sample. In some embodiments, a complement of SEQ ID NO:35 (5′-UACCCUGUAGAACCGAAUUUGUG′3′) is used to measure expression of naturally occurring miR-10b in a sample. In some embodiments, a complement of SEQ ID NO:36 (5′-UAGCAGCACAUAAUGGJUUULGUG′3′) is used to measure expression of naturally occurring miR-15a in a sample. In some embodiments, a complement of SEQ ID NO:37 (5′-UGCCUACUiAGCUGAUAUCAGU′3′) is used to measure expression of naturally occurring miR-24-1* in a sample. In some embodiments, a complement of SEQ ID NO:38 (5′-UGCCUACUGAGCUGAAACACAG′3′) is used to measure expression of naturally occurring miR-24-2* in a sample. In some embodiments, a complement of SEQ ID NO:39 (5′-LRICACAGUGGCUAAGUUCCGC′3′) is used to measure expression of naturally occurring miR-27a in a sample. In some embodiments, a complement of SEQ ID NO:40 (5′-AGAGCUUAGCUGAUUGGUGAAC′3′) is used to measure expression of naturally occurring miR-27b* in a sample. In some embodiments, a complement of SEQ ID NO:41 (5′-UGCUAUGCCAACAUAUUGCCAU′3′) is used to measure expression of naturally occurring miR-31* in a sample. In some embodiments, a complement of SEQ ID NO:42 (5′-CAAUCAGCAAGUAUACUGCCCU′3′) is used to measure expression of naturally occurring miR-34a* in a sample. In some embodiments, a complement of SEQ ID NO:43 (5′-UAGGCAGUGUCAUUAGCUGAUUG′3′) is used to measure expression of naturally occurring miR-34b* in a sample. In some embodiments, a complement of SEQ ID NO:44 (5′-UAACAGUCUACACCAGCCAUGGUCG′3′) is used to measure expression of naturally occurring miR-132 in a sample. In some embodiments, a complement of SEQ ID NO:45 (5′-ACCGUGGCUUUCGAUUGUUACU′3′) is used to measure expression of naturally occurring miR-132* in a sample. In some embodiments, a complement of SEQ ID NO:46 (5′-CAUAAAGUAGAAAGCACUACU′3′) is used to measure expression of naturally occurring miR-140-5p in a sample. In some embodiments, a complement of SEQ ID NO:47 (5′-ACCAUCGACCCJUUGAUUGUACC′3′) is used to measure expression of naturally occurring miR-181a* in a sample. In some embodiments, a complement of SEQ ID NO:48 (5′-AACAUUCAACCUGUCGGUGAGU′3′) is used to measure expression of naturally occurring miR-181c in a sample. In some embodiments, a complement of SEQ ID NO:49 (5′-UUUGGCAAUGGUAGAACUCACACU′3′) is used to measure expression of naturally occurring miR-182 in, a sample. In some embodiments, a complement of SEQ ID NO:50 (5′-UAGGUAGUUUCAUGUUGUUGGG′3′) is used to measure expression of naturally occurring milt-196a in a sample. In some embodiments, a complement of SEQ ID NO:51 (5′-CAGUGCAAUAGUAUUGUCAAAGC′3′) is used to measure expression of naturally occurring miR-301a in a sample. In some embodiments, a complement of SEQ ID NO:52 (5′-UCAAGAGCAAUAACOAAAAAUGU′3′) is used to measure expression of naturally occurring miR-335 in a sample. In some embodiments, a complement of SEQ ID NO:53 (5′-UUAUAAAGCAAUGAGACUGAUU′3′) is used to measure expression of naturally occurring miR-340 in a sample. In some embodiments, a complement of SEQ ID NO:54 (5′-GCGACCCAUACUUGGUUUCAG′3′) is used to measure expression of naturally occurring miR-551b in a sample. In some embodiments, a complement of SEQ ID NO:55 (5′-UGAGGCAGUAGAUUGAAU′3′) is used to measure expression of naturally occurring miR-1827 in a sample. In some embodiments, a complement of SEQ ID NO:56 (5′-UCAACAAAAUCACUGAUGCUGGA′3′) is used to measure expression of naturally occurring miR-3065-5p in a sample. In some embodiments, a complement of SEQ ID NO:57 (5′-UUGUCUGCUGAGUUUCC′3′) is used to measure expression of naturally occurring miR-4288 in a sample. In some embodiments, a complement of SEQ ID NO:63 (5′ CAUAAAGUAGAAAGCACUACU 3′) is used to measure expression of naturally occurring miR-142-5p in a sample. In some embodiments, a complement of SEQ ID NO:64 (5′-GUCCCUGUUCAGGCGCCA′3′) is used to measure expression of naturally occurring miR-1274a in a sample. In some embodiments, a complement of SEQ ID NO:90 (5′-UGUGCAAAUCCAUGCAAAACUGA′3′) is used to measure expression of naturally occurring miR-19b-3p in a sample. In some embodiments, a complement of SEQ ID NO:129 (5′-UUUGGCACUAGCACAUUUGCU′3′) is used to measure expression of naturally occurring miR-96-5p in a sample. In some embodiments, a complement of SEQ ID NO:139 (5′-UAGCACCAUUUGAAAUCAGUGUU′3′) is used to measure expression of naturally occurring miR-29b-3p in a sample. In some embodiments, a complement of SEQ ID NO:178 (5′-ACCACUGACCGUUGACUGUACC′3′) is used to measure expression of naturally occurring miR-181a-2-3p in a sample. In some embodiments, a complement of SEQ ID NO:192 (5′-UUCCCUUUGUCAUCCUAUGCCU′3′) is used. to measure expression of naturally occurring miR-204-5p in a sample. In some embodiments, a complement of SEQ ID NO:388 (5′-CUAGACUGAAGCUCCUUGAGG′3′) is used to measure expression of naturally occurring miR-151a-3p in a sample. In some embodiments, a complement of SEQ ID NO:413 (5′-CAGCAGCAAUUCAUGUUUUGAA′3′) is used to measure expression of naturally occurring miR-424-5p in a sample. It is contemplated that a probe used in methods may be 85, 86, 87, 88, 89, 90, 91, 92, 93, 94, 95, 96, 97, 98, 99, or 100% complementary (and any range derivable therein) to any of SEQ ID NOs:1-64 or any sequence found herein. Such probes may be 10, 11, 12, 13, 14, 15, 16, 17, 18, 19, 20, 21, 22, 23, 24, 25, 26, 27, 28, 29, or 30 residues in length (or any range derivable therein).

ft is contemplated that the target of a miRNA may be measured as a surrogate of miRNA expression levels. For example, a target for miR-221 and miR-222 is CDKN1B (p27^{Kip1}).

Methods may also involve one or more of the following steps: obtaining a thyroid sample of a patient; preparing the sample to characterize miRNA in the sample (for instance, for hybridization and/or amplification); storing a sample from a patient; assessing the integrity or adequacy of the sample, such as of the nucleic acids; doing a cytology analysis of the sample; staining all or part of the sample using tissue stains; fixing all or part of the sample; freezing all or part of the sample; transporting the sample; providing or being provided one or more images of the sample; visually accessing the sample directly or remotely, such as with telemedicine; measuring the level of expression. of at least one biomarker miRNAs; comparing the level of expression of each biomarker miRNA to the level of expression of another biomarker miRNA; calculating a diagnostic score that indicates the probability the thyroid sample is benign or is malignant, wherein the diagnostic score is based on comparisons between the expression levels of the biomarker miRNAs to the expression level of at least one other biomarker miRNA; reporting the diagnostic score or the probability the thyroid sample is benign or malignant; storing information related to the expression levels of measured miRNA or related to comparisons between levels of miRNA or related to calculations from measured and/or compared expression levels or related to diagnostic scores; implementing an algorithm on a computer to calculate values that reflect comparisons between or among expression levels of biomarker miRNAs; implementing an algorithm on a computer to calculate diagnostic scores; diagnosing the patient as having or not having a thyroid malignancy; diagnosing the patient as having or not having a benign thyroid condition; categorizing a thyroid sample as a particular subtype or category of malignant thyroid conditions; categorizing a thyroid sample as a particular subtype or category of benign thyroid conditions; monitoring a patient's thyroid; and/or, treating the patient for a thyroid malignancy. It is contemplated that the score may indicate the probability that the sample is benign. In other embodiments, the score may indicate the probability that the sample is malignant. It will also be understood that a sample may be tested or evaluated more than one time either at the same time and/or at different times. In some cases, another test is run to obtain a second opinion on the same sample or a different sample from the patient.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to a level of expression in a thyroid sample from a patient of at least two of the following diff pair miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-197a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, or miR-182-5p, wherein at least one of the miRNAs is a biomarker miRNA; and b) determining a biomarker diff pair value using information corresponding to the at least one biomarker miRNA and information corresponding to the level of expression of a comparative microRNA, the diff pair value being indicative of whether the thyroid growth is malignant or benign. In some embodiments, receiving information comprises receiving from a tangible data storage device information corresponding to a level of expression in a thyroid sample from a patient of at least two of the following diff pair miRNAs: miR-375, miR-146b-3p, miR-146b-5p, miR-221-3p, miR-222-3p, miR-551b-3p, miR-204-5p, miR-7-5p, miR-141-3p, miR-31-5p, miR-18.1a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, or miR-182-5p, wherein at least one of the miRNAs is a biomarker miRNA. In additional embodiments, information is used that corresponds to the level of expression of a comparative miRNA that is miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, or miR-182-5p. In additional embodiments the medium further comprises computer-readable code that, when executed by a computer, causes the computer to perform one or more additional operations comprising: sending information corresponding to the biomarker diff pair value to a tangible data storage device. In specific embodiments, it further comprises computer-readable code that, when executed by a computer, causes the computer to perform one or more additional operations comprising: sending information corresponding to the biomarker diff pair value to a tangible data storage device. In certain embodiments, receiving information comprises receiving from a tangible data storage device information corresponding to a level of expression in a thyroid sample from a patient of at least two of the following diff pair miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, or miR-182-5p, wherein at least one of the miRNAs is a biomarker miRNA. In even further embodiments, the tangible computer-readable medium has computer-readable code that, when executed by a computer, causes the computer to perform operations further comprising: c) calculating a diagnostic score for the thyroid sample, wherein the diagnostic score is indicative of the probability that the thyroid sample is malignant. it is contemplated that any of the methods described above may be implemented, with tangible computer readable medium that has computer readable code, that when executed by a computer, causes the computer to perform operations related to the measuring, comparing, and/or calculating a diagnostic score related to the probability of a malignancy or a benign thyroid condition.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data.

Also provided are kits containing the disclosed compositions or compositions used to implement the disclosed methods. In some embodiments, kits can be used to evaluate one or more miRNA molecules. In certain embodiments, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34,35, 36, 37, 38, 39, 40, 41, 42, 43, 44, 45, 46, 47, 48, 49, 50 or more, or any range and combination derivable therein, miRNA probes including those that may specifically hybridize under stringent conditions to miRNAs disclosed. herein. In other embodiments, kits or methods may involve 1, 2, 3, 4, 5, 6, 7, 8, 9, 10, 11, or 12 or more (or any range derivable therein) miRNA probes or corresponding primers, which may be capable of specifically detecting any of the following miRNAs: miR-375, miR-146b-3p, miR-146b-5p, miR-221-3p, miR-222-3p, miR-551b-3p, miR-204-5p, miR-7-5p, miR-141-3p, miR-31-5p, miR-181a-2-3p, miR-15a-5p, miR-224-5p, miR-221-5p, miR-222-5p, miR-21-5p, miR-424-5p, miR-34a-5p, miR-197-3p, miR-19b-3p, miR-155-5p, miR-138-1-3p, miR-139-5p, miR-151a-3p, miR-29b-3p, miR-96-5p, miR-34a-3p, miR-3065-5p, miR-34b-5p, miR-31-3p, miR-146a, miR-181c, or miR-182-5p.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any disclosed method or composition, and vice versa. Any embodiment discussed with respect to a method can be implemented in the context of a kit or computer readable medium. Furthermore, the disclosed compositions and kits can be used to achieve the disclosed methods.

Throughout this application, the term “about” is used to indicate that a value includes the standard deviation of error for the device or method being employed to determine the value.

The use of the term “or” in the claims is used to mean “and/or” unless explicitly indicated to refer to alternatives only, or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. However, for a claim using any of these terms, embodiments are also contemplated where the claim is closed and does exclude additional, unrecited elements or method steps.

Other objects, features and advantages of the invention will be apparent from the following detailed description. It should be understood, however, that the detailed description and the specific examples, while indicating specific embodiments, are given by way of illustration only, because various changes and modifications within the spirit and scope of the invention will be apparent to those skilled in the art from this detailed description.

### DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIGS. 1A, 1B and IC. Differentially expressed miRNAs identified from FFPE Samples. (A) 18 Differentially expressed miRs identified by LIMMA ANOVA analysis of Affymeirix miRNA array data (all significant at p<0.05 after Benjamini-Hochberg FDR adjustment). This analysis, in conjunction with pair-wise comparisons between different sample subgroups, led to the identification of a set of miRNAs for qRT-PCR Analysis. Good correlation was observed for benign-vs-malignant fold changes (shown as log-ratios for array and delta-Ct for PCR) between the array data and the PCR data for selected miRNA chosen for assay in FNA set 1 (B) and FNA set 2 (C). B=benign conditions other than FA.

FIGS. 2A and 2B, miRNA Expression in FNA Specimens correlated with mutational status. Principal Components Analysis (PCA) applied to qRT-PCR data (raw Ct values) derived from (A) PNA set 1 and (B) FNA set 2. Both sets show clear separation of BRAF-mutation positive samples from benign, less separation between mutation-negative malignant samples and benign samples. B_neg=benign, mutation-negative; M_BRAF=malignant, V600E mutation-positive; M_RAS=malignant, RAS mutation-positive; M_neg=malignant, mutation-negative.

FIG. 3. Preliminary Logistic Regression Classifier for FNA Samples, miR-146b and miR-222 show strongly differential expression between BRAF WT and mutant populations, but are much less useful for distinguishing mutation negative malignant from benign specimens.

FIGS. 4A, 4B and 4C. Strongest miRNA signals in miRNAs correlate with BRAF status. Logistic model fit to merged FNA set (using 11 miRNA, species assayed on both FNA sets). Model was fit using λ₁ penalized regression to DiffPaired data. The performance of this model was assessed using 5-fold cross-validation, with resulting malignancy predictions shown in (A) and resulting ROC curve shown in (B). Four DiffPairs received non-zero weights in final model, with the top two DiffPairs (values shown in (C)) having >5-fold larger weights than the bottom two.

FIG. 5. Algorithm describing the approach for modeling the distinction between benign and malignant FNA samples.

FIG. 6. LOOCV classification scores produced separating Hashimoto's disease samples from other thyroid samples.

FIG. 7. LOOCV classification scores produced by 5-miRNA model.

FIG. 8. LOOCV classification scores produced by 10-miRNA model.

### DETAILED DESCRIPTION OF THE INVENTION

Certain embodiments are directed to compositions and methods relating to preparation and characterization of miRNAs, as well as use of miRNAs for prognostic and diagnostic applications, particularly those methods and compositions related to assessing and/or identifying thyroid cancer.

Histological subtypes of thyroid cancers have distinct miRNA expression profiles, and recent studies have suggested that miRNAs may be used in preoperative FNA samples to distinguish between hyperplastic thyroid nodules and malignant thyroid tumors. The use of miRNAs, as described herein, will complement and improve the performance (sensitivity and NPV) of the mutation Panel assay and allow the accurate classification of malignant FNAs, including those that are mutation-negative.

### I. miRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enable them to form a stem-loop- or fold-back-like structure, which is cleaved in animals by a ribonuclease Ill-like nuclease enzyme called Dicer. The processed miRNA is typically a portion of the stem.

The processed miRNA (also referred to as “mature miRNA”) becomes part of a large complex to down-regulate a particular target gene. Examples of animal miRNAs include those that imperfectly basepair with the target, which halls translation of the target (Olsen et al., 1999; Seggerson et al., 2002). siRNA molecules also are processed by Dicer, but from a long, double-stranded RNA. molecule. siRNAs are not naturally found in animal cells, but they can direct the sequence-specific cleavage of an mRNA target through an RNA-induced silencing complex (RISC) (Denli et al., 2003). In some embodiments, the mature miRNAs may be any of the miRNAs listed in Table 1.

TABLE 1

| | | SEQ ID |
|---|---|---|
| miRNA | Probe Sequence | NO |
| hsa-miR- | GGAGAAAUUAUCCUUGGUGUGU | SEQ ID |
| 539 | | NO: 1 |
| hsa-miR- | UGGAAGACUAGUGAUUUUGUUGU | SEQ ID |
| 7 | | NO: 2 |
| hsa-miR- | UGAGAACUGAAUUCCAUAGGCU | SEQ ID |
| 146b-5p | | NO: 3 |
| hsa-miR- | UGCCCUGUGGACUCAGUUCUGG | SEQ ID |
| 146b-3p | | NO: 4 |
| hsa-miR- | AACACACCUGGUUAACCUCUUU | SEQ ID |
| 329 | | NO: 5 |
| hsa-miR- | UCGGAUCCGUCUGAGCUUGGCU | SEQ ID |
| 127-3p | | NO: 6 |
| hsa-miR- | ACCUGGCAUACAAUGUAGAUUU | SEQ ID |
| 221* | | NO: 7 |
| hsa-miR- | CCAGUCCUGUGCCUGCCGCCU | SEQ ID |
| 1910 | | NO: 8 |
| hsa-miR- | AAACAAACAUGGUGCACUUCUU | SEQ ID |
| 495 | | NO: 9 |
| hsa-miR- | GAAUGUUGCUCGGUGAACCCCU | SEQ ID |
| 409-3p | | NO: 10 |
| hsa-miR- | AACAUAGAGGAAAUUCCACGU | SEQ ID |
| 376c | | NO: 11 |
| hsa-miR- | UCCUUCAUUCCACCGGAGUCUG | SEQ ID |
| 205 | | NO: 12 |
| hsa-miR- | UUUGUUCGUUCGGCUCGCGUGA | SEQ ID |
| 375 | | NO: 13 |
| hsa-miR- | CUCAGUAGCCAGUGUAGAUCCU | SEQ ID |
| 222* | | NO: 14 |
| hsa-miR- | AGAUCGACCGUGUUAUAUUCGC | SEQ ID |
| 369-5p | | NO: 15 |
| hsa-miR- | UGUCUGCCCGCAUGCCUGCCUCU | SEQ ID |
| 346 | | NO: 16 |
| hsa-miR- | AGCUACAUUGUCUGCUGGGUUUC | SEQ ID |
| 221 | | NO: 17 |
| hsa-miR- | AGGCAAGAUGCUGGCAUAGCU | SEQ ID |
| 31 | | NO: 18 |
| hsa-miR- | UUGUACAUGGUAGGCUUUCAUU | SEQ ID |
| 493* | | NO: 19 |
| hsa-miR- | AAUCUGAGAAGGCGCACAAGGU | SEQ ID |
| 3200-5p | | NO: 20 |
| hsa-miR- | CACCUUGCGCUACUCAGGUCUG | SEQ ID |
| 3200-3p | | NO: 21 |
| hsa-miR- | UAGCUUAUCAGACUGAUGUUGA | SEQ ID |
| 21 | | NO: 22 |
| hsa-miR- | UGGCAGUGUCUUAGCUGGUUGU | SEQ ID |
| 34a | | NO: 23 |
| hsa-miR- | GCUACUUCACAACACCAGGGCC | SEQ ID |
| 138-1* | | NO: 24 |
| hsa-miR- | UCUACAGUGCACGUGUCUCCAG | SEQ ID |
| 139-5p | | NO: 25 |
| hsa-miR- | UAACACUGUCUGGUAAAGAUGG | SEQ ID |
| 141 | | NO: 26 |
| hsa-miR- | GUCCAGUUUUCCCAGGAAUCCCU | SEQ ID |
| 145 | | NO: 27 |
| hsa-miR- | UUAAUGCUAAUCGUGAUAGGGGU | SEQ ID |
| 155 | | NO: 28 |
| hsa-miR- | UAUGGCACUGGUAGAAUUCACU | SEQ ID |
| 183 | | NO: 29 |
| hsa-miR- | UUCACCACCUUCUCCACCCAGC | SEQ ID |
| 197 | | NO: 30 |
| hsa-miR- | AGCUACAUCUGGCUACUGGGU | SEQ ID |
| 222 | | NO: 31 |
| hsa-miR- | CAAGUCACUAGUGGUUCCGUU | SEQ ID |
| 224 | | NO: 32 |
| hsa-miR- | AAUGACACGAUCACUCCCGUUGA | SEQ ID |
| 425 | | NO: 33 |
| hsa-miR- | UGAGAACUGAAUUCCAUGGGUU | SEQ ID |
| 146a | | NO: 34 |
| hsa-miR- | UACCCUGUAGAACCGAAUUUGUG | SEQ ID |
| 10b | | NO: 35 |
| hsa-miR- | UAGCAGCACAUAAUGGUUUGUG | SEQ ID |
| 15a | | NO: 36 |
| hsa-miR- | UGCCUACUGAGCUGAUAUCAGU | SEQ ID |
| 24-1* | | NO: 37 |
| hsa-miR- | UGCCUACUGAGCUGAAACACAG | SEQ ID |
| 24-2* | | NO: 38 |
| hsa-miR- | UUCACAGUGGCUAAGUUCCGC | SEQ ID |
| 27a | | NO: 39 |
| hsa-miR- | AGAGCUUAGCUGAUUGGUGAAC | SEQ ID |
| 27b* | | NO: 40 |
| hsa-miR- | UGCUAUGCCAACAUAUUGCCAU | SEQ ID |
| 31* | | NO: 41 |
| hsa-miR- | CAAUCAGCAAGUAUACUGCCCU | SEQ ID |
| 34a* | | NO: 42 |
| hsa-miR- | UAGGCAGUGUCAUUAGCUGAUUG | SEQ ID |
| 34b* | | NO: 43 |
| hsa-miR- | UAACAGUCUACAGCCAUGGUCG | SEQ ID |
| 132 | | NO: 44 |
| hsa-miR- | ACCGUGGCUUUCGAUUGUUACU | SEQ ID |
| 132* | | NO: 45 |
| hsa-miR- | CAUAAAGUAGAAAGCACUACU | SEQ ID |
| 140-5p | | NO: 46 |
| hsa-miR- | ACCAUCGACCGUUGAUUGUACC | SEQ ID |
| 181a | | NO: 47 |
| hsa-miR- | AACAUUCAACCUGUCGGUGAGU | SEQ ID |
| 181c | | NO: 48 |
| hsa-miR- | UUUGGCAAUGGUAGAACUCACACU | SEQ ID |
| 182 | | NO: 49 |
| hsa-miR- | UAGGUAGUUUCAUGUUGUUGGG | SEQ ID |
| 196a | | NO: 50 |
| hsa-miR- | CAGUGCAAUAGUAUUGUCAAAGC | SEQ ID |
| 301a | | NO: 51 |
| hsa-miR- | UCAAGAGCAAUAACGAAAAAUGU | SEQ ID |
| 335 | | NO: 52 |
| hsa-miR- | UUAUAAAGCAAUGAGACUGAUU | SEQ ID |
| 340 | | NO: 53 |
| hsa-miR- | GCGACCCAUACUUGGUUUCAG | SEQ ID |
| 551b | | NO: 54 |
| hsa-miR- | UGAGGCAGUAGAUUGAAU | SEQ ID |
| 1827 | | NO: 55 |
| hsa-miR- | UCAACAAAAUCACUGAUGCUGGA | SEQ ID |
| 3065-5p | | NO: 56 |
| hsa-miR- | UUGUCUGCUGAGUUUCC | SEQ ID |
| 4288 | | NO: 57 |
| hsa-miR- | AGGGGCUGGCUUUCCUCUGGUC | SEQ ID |
| 185* | | NO: 58 |
| hsa-miR- | CAGAGAAUUGUUUAAUC | SEQ ID |
| 3123 | | NO: 59 |
| hsa-miR- | CCAGUCCUGUGCCUGCCGCCU | SEQ ID |
| 1910 | | NO: 60 |
| hsa-miR- | UCCUGUACUGAGCUGCCCCGAG | SEQ ID |
| 486-5p | | NO: 61 |
| hsa-miR- | AAAGUGCAUCCUUUUAGAGUGU | SEQ ID |
| 519a | | NO: 62 |
| hsa-miR- | CAUAAAGUAGAAAGCACUACU | SEQ ID |
| 142-5p | | NO: 63 |
| hsa-miR- | GUCCCUGUUCAGGCGCCA | SEQ ID |
| 1274a | | NO: 64 |
| hsa-miR- | UGUGCAAAUCCAUGCAAAACUGA | SEQ ID |
| 19b-3p | | NO: 90 |
| hsa-miR- | UUUGGCACUAGCACAUUUUUGCU | SEQ ID |
| 96-5p | | NO: 129 |
| hsa-miR- | UAGCACCAUUUGAAAUCAGUGUU | SEQ ID |
| 29b-3p | | NO: 139 |
| hsa-miR- | ACCACUGACCGUUGACUGUACC | SEQ ID |
| 181a- | | NO: 178 |
| 2-3p | | |
| hsa-miR- | UUCCCUUUGUCAUCCUAUGCCU | SEQ ID |
| 204-5p | | NO: 192 |
| hsa-miR- | CUAGACUGAAGCUCCUUGAGG | SEQ ID |
| 151a-3p | | NO: 388 |
| hsa-miR- | CAGCAGCAAUUCAUGUUUUGAA | SEQ ID |
| 424-5p | | NO: 413 |

A. Nucleic Acids

In the disclosed compositions and methods miRNAs can be labeled, used in. array analysis, or employed in diagnostic, therapeutic, or prognostic applications, particularly those related to pathological conditions of the thyroid. The RNA may have been endogenously produced by a cell, or been synthesized or produced chemically or recombinantly. They may be isolated and/or purified. The term “miRNA,” unless otherwise indicated, refers to the single-stranded processed RNA, after it has been cleaved from its precursor. The name of the miRNA is often abbreviated and referred to without a hsa-, mmu-, or mo-prefix and will be understood as such, depending on the context. Unless otherwise indicated, miRNAs referred to are human sequences identified as miR-X or let-X, where X is a number and/or letter.

In certain experiments, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the World Wide Web at sanger.ac.uk. Moreover, in some embodiments, a miRNA probe is used that does not correspond to a known human miRNA. It is contemplated that these non-human miRNA probes may be used in embodiments or that there may exist a human miRNA that is homologous to the non-human miRNA. While the methods and compositions are not limited to human miRNA, in certain embodiments, miRNA from human cells or a human biological sample is used or evaluated. In other embodiments, any mammalian miRNA or cell, biological sample, or preparation thereof may be employed.

In some embodiments, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 0.19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56,57, 58, 59, 60, 61, 62, 63, 64, 65, 66, 67, 68, 69, 70, 71, 72,73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA. containing vectors, control nucleic acids, and other probes and primers. In many embodiments, miRNAs are 19-24 nucleotides in length, while miRNA probes are 19-35 nucleotides in length, depending on the length of the processed miRNA and any flanking regions added. mRNA precursors are generally between 62 and 110 nucleotides in human s.

Nucleic acids used in methods and compositions disclosed herein may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000, or any range derivable therein, contiguous nucleotides from any nucleic acid discussed or provided herein, including any miR or precursor miR sequence. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of the sequences disclosed herein. The commonly used name of the miRNA is given (with its identifying source in the prefix, for example, “hsa” for human sequences) and the processed miRNA sequence. Unless otherwise indicated, a miRNA without a prefix will be understood to refer to a human miRNA. A miRNA designated, for example, as miR-1-2 in the application will be understood to refer to hsa-miR-1-2. Moreover, a lowercase letter in the name of a miRNA may or may not be lowercase; for example, hsa-mir-130b can also be referred to as miR-130B. In addition, miRNA sequences with a “mu” or “mmu” sequence will be understood to refer to a mouse miRNA and miRNA sequences with a “mo” sequence will be understood to refer to a rat miRNA. The term “miRNA probe” refers to a nucleic acid probe that can identify a particular miRNA or structurally related miRNAs.

It is understood that a miRNA is derived from genomic sequences or a gene. In this respect, the term “gene” is used for simplicity to refer to the genomic sequence encoding the precursor miRNA for a given miRNA. However, embodiments may involve genomic sequences of a miRNA that are involved in its expression, such as a promoter or other regulatory sequences.

The term “recombinant” generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids can include, can be, or can be at least 60, 65, 70, 75, 80, 85, 90, 95, 96, 97, 98, 99 or 100% complementary to their target.

As used herein, “hybridization”, “hybridizes” or “capable of hybridizing” is understood to mean the forming of a double or triple stranded molecule or a molecule with partial double or triple stranded nature. The term “anneal” is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein, “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 NM to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of such include hybridization performed at about 0.15 M to about 0.9 M NaCl at a temperature range of about 20° C. to about 50° C. Of course, it is within the skill of one in the art to further modify the low or high stringency conditions to suite a particular application.

Embodiments may concern detecting miRNA expression levels through hybridization such as with probes. In other embodiments, expression levels may be determined using PCR, such as quantitative PCR. Primers to amplify target miRNA sequences (or corresponding cDNA sequences) may be employed in some embodiments.

Labeling methods and kits may use nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the methods and compositions are not naturally occurring nucleotides, but instead, refer to prepared nucleotides that have a reactive moiety on them. Specific reactive functionalities of interest include: amino, sulfhydryl, sulfoxyl, aminosulthydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide; glyoxal, aldehyde, iodoacetyl, cyanomethyl ester, p-nitrophenyl. ester, o-nitrophenyl ester, hydroxypyridine ester, carbonyl imidazole, and other such chemical. groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in some embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, etc. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in some embodiments. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amnino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dCTP, dTTP, or dUTP in place of a 5-(3-aminoallyl)-UTP.

### B. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased.

Methods may involve the use of organic solvents and/or alcohol to isolate nucleic acids, particularly miRNA used in methods and compositions disclosed herein. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column may work particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, 1) capturing the miRNA molecules. Typically the sample is dried down and resuspended in a liquid and volume appropriate for subsequent manipulation.

### II. LABELS AND LABELING TECHNIQUES

In some embodiments, miRNAs are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In particular embodiments, the label is non-radioactive. Generally, nucleic acids may be labeled by adding labeled nucleotides (one-step process) or adding nucleotides and labeling the added nucleotides (two-step process).

### A. Labeling Techniques

In some embodiments, nucleic acids are labeled by catalytically adding to the nucleic acid an already labeled nucleotide or nucleotides. One or more labeled nucleotides can be added to miRNA-molecules. See U.S. Pat. No. 6,723,509, which is hereby incorporated by reference.

In other embodiments, an unlabeled nucleotide(s) is catalytically added to a miRNA, and the unlabeled nucleotide is modified with a chemical moiety that enables it to be subsequently labeled. In some embodiments, the chemical moiety is a reactive amine such that the nucleotide is an amine-modified nucleotide. Examples of amine-modified nucleotides are well known to those of skill in the art, many being commercially available.

In contrast to labeling of cDNA during its synthesis, the issue for labeling miRNA is how to label the already existing molecule. Some aspects concern the use of an enzyme capable of using a di- or tri-phosphate ribonucleotide or deoxyribonucleotide as a substrate for its addition to a miRNA. Moreover, in specific embodiments, a modified di- or tri-phosphate ribonucleotide is added to the 3′ end of a miRNA. The source of the enzyme is not limiting. Examples of sources for the enzymes include yeast, gram-negative bacteria such as E. coli, Lactococcus lactis, and sheep pox virus.

Enzymes capable of adding such nucleotides include, but are not limited to, poly(A) polymerase, terminal transferase, and polynucleotide phosphorylase. In specific embodiments, a ligase is contemplated as not being the enzyme used to add the label, and instead, a non-ligase enzyme is employed.

Terminal transferase may catalyze the addition of nucleotides to the 3′ terminus of a nucleic acid. Polynucleotide phosphorylase can polymerize nucleotide diphosphates without the need for a primer.

### B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue: Cascade Yellow; coumarin and its derivatives, such as 7-amino-4-methylcoumiarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dyer™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Flour 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides include Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UCP, and BODIPY TR-14-UTP. Other fluorescent ribonucleotides include Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP.

It is contemplated that nucleic acids may be labeled with two different labels. Furthermore, fluorescence resonance energy transfer (FRET) may be employed in disclosed methods (e.g., Klostermeier et al., 2002; Emptage, 2001; Didenko, 2001, each incorporated by reference).

Alternatively, the label, may not be detectable per se, but indirectly detectable or allowing for the isolation or separation of the targeted nucleic acid. For example, the label could be biotin, digoxigenin, polyvalent cations, chelator groups and other ligands, include ligands for an antibody.

### C. Visualization Techniques

A number of techniques for visualizing or detecting labeled nucleic acids are readily available. Such techniques include, microscopy, arrays, fluorometry, light cyclers or other real time PCR machines, FACS analysis, scintillation counters, phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acids. Furthermore, a person of ordinary skill in the art is well aware of ways of visualizing, identifying, and characterizing labeled nucleic acids, and accordingly, such protocols may be used. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### III. ARRAY PREPARATION AND SCREENING

A. Array Preparation

Some embodiments involve the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, and silicon. Such arrays may vary in a number of different ways, including average probe length, sequence or types of probes, nature of bond between the probe and the array surface, e.g. covalent or non-covalent, and the like. The labeling and screening methods are not limited by with respect to any parameter except that the probes detect miRNA; consequently, methods and compositions may be used with a variety of different types of miRNA. arrays.

Representative methods and apparatuses for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/1995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898: WO 03040410; WO 03053586; WO 03087297; WO 03091426; WO03100012; WO 04020085; WO 04027093; EP 373 203; EP 785 280; EP 799 897 and UK 8 803 000, which are each herein incorporated by reference.

It is contemplated that the arrays can be high density arrays, such that they contain 2, 20, 25, 50, 80, 100, or more, or any integer derivable therein, different probes. It is contemplated that they may contain 1000, 16,000, 65,000, 250,000 or 1,000,000 or more, or any integer or range derivable therein, different probes. The probes can be directed to targets in one or more different organisms or cell types. In some embodiments, the oligonucleotide probes may range from 5 to 50, 5 to 45, 10 to 40, 9 to 34, or 15 to 40 nucleotides in length. In certain embodiments, the oligonucleotide probes are 5, 10, 15, 20, 25, 30, 35, 40 nucleotides in length, including all integers and ranges there between.

Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed herein or may be found in, for example, WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the miRNA of a wide variety of samples can be analyzed using arrays, miRNA probes, or array technology. While endogenous miRNA is contemplated for use with compositions and methods disclosed herein, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells. In certain embodiments, samples may be, but are not limited to, fresh, frozen, fixed, formalin fixed, paraffin embedded, or formalin fixed and paraffin embedded. Alternatively, the sample may not be a biological sample, but a chemical mixture, such as a cell-free reaction mixture (which may contain, one or more biological enzymes).

1. Biological Sample Collection

In certain aspects, methods involve obtaining a sample from a subject. The term subject may refer to an animal (for example a mammal), including but not limited to humans, non-human primates, rodents, dogs, or pigs. The methods of obtaining provided herein include methods of biopsy such as fine needle aspiration, core needle biopsy, vacuum assisted biopsy, incisional biopsy, excisional biopsy, punch biopsy, shave biopsy or skin biopsy: The sample may be obtained from any of the tissues provided herein that include but are not limited to gall bladder, skin, heart, lung, breast, pancreas, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples, such as multiple thyroid samples may be obtained for diagnosis by the methods described herein. In other cases, multiple samples, such as one or more samples from one tissue type (for example thyroid) and one or more samples from another tissue (for example, buccal) may be obtained for diagnosis by the methods of the present methods. In some cases, multiple samples such as one or more samples from one tissue type (e.g., thyroid) and one or more samples from another tissue (e.g., buccal) may be obtained at the same or different times. Samples may be obtained at different times are stored and/or analyzed by different methods. For example, a sample may be obtained and analyzed by routine staining methods or any other cytological analysis methods.

In some cases, further samples may be obtained from a subject based on the results of such a cytological analysis. A cancer diagnosis may include an examination of a subject by a physician, nurse or other medical professional. The examination may be part of a routine examination, or the examination may be due to a specific complaint. A specific complaint may include but is not limited to: pain, illness, anticipation of illness, presence of a suspicious lump or mass, a disease, or a condition.

In some embodiments the subject may or may not be aware of the disease or condition.

In some cases, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated.

In other cases, the sample is obtained by an invasive procedure including but not limited to: biopsy, alveolar or pulmonary lavage, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material. Methods of obtaining suitable samples of thyroid are known in the art and are further described in the ATA Guidelines for thyroid nodule management (Cooper et al. Thyroid Vol. 16 No. 2 2006), herein incorporated by reference in its entirety.

General methods for obtaining biological samples are also known in the art. Publications such as Ramzy, Ibrahim Clinical Cytopathology and Aspiration Biopsy 2001, which is herein incorporated by reference in its entirety, described general methods for biopsy and cytological methods. In one embodiment, the sample is a fine needle aspirate of a thyroid nodule or a suspected thyroid tumor. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described. herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

2. Biological Sample Storage

In certain aspects, a sample may be obtained and prior to analysis by one or more methods described herein, the sample may be stored for a length of time. A length of time may include a time interval such as seconds, minutes, hours, days, weeks, months, years or longer. In some cases, the sample obtained from a subject is subdivided prior to the step of storage or further analysis. In some cases where the sample is subdivided different portions of the sample are subjected to different downstream methods or processes. Such methods or processes may include storage, cytological analysis, integrity tests, nucleic acid extraction, molecular profiling or any combination of these.

In some cases where storage is contemplated, some part of the sample may be stored while another portion of the sample is further processed. Processing may include but is not limited to molecular profiling, cytological staining, gene or gene expression product (RNA or protein) extraction, detection, or quantification, fixation or examination.

In other cases, the sample is obtained and stored and subdivided, after the step of storage for further analysis such that different portions of the sample are subject to different downstream methods or processes including but not limited to storage, cytological, analysis, adequacy tests, nucleic acid extraction, molecular profiling or a combination thereof.

In some cases, samples are obtained and analyzed by cytological analysis, and the resulting sample material is further analyzed by one, or more molecular profiling methods described herein. In such cases, the samples may be stored between the steps of cytological analysis and the steps of molecular profiling. Samples may be stored upon acquisition to facilitate transport, or to wait for the results of other analyses. In another embodiment, samples may be stored while awaiting instructions a medical professional.

An acquired sample may be placed in short term or long term storage by placing in a suitable medium, excipient, solution, or container. In certain cases storage may require keeping the sample in a refrigerated, or frozen environment. The sample may be quickly frozen prior to storage in a frozen environment. In certain instances the frozen sample may be contacted with a suitable cryopreservation medium or compound. Examples of cryopreservation mediums or compounds include but are not limited to: glycerol, ethylene glycol, sucrose, or glucose.

A suitable medium, excipient, or solution may include but is not limited to: hanks salt solution, saline, cellular growth medium, an ammonium salt solution such as ammonium sulphate or ammonium phosphate, or water.

Suitable concentrations of ammonium salts include solutions of about 0.1 g/ml, 0.2 g/ml, 0.3 g/ml, 0.4 g/ml, 0.5 g/ml, 0.6 g/ml, 0.7 g/ml, 0.8 g/ml, 0.9 g/ml, 1.0 g/ml, 1.1 g/ml, 1.2 g/ml, 1.3 g/ml, 1.4 g/ml, 1.5 g/ml, 1.6 g/ml, 1.7 g/ml, 1.8 g/ml, 1.9 g/ml, 2.0 g/ml, 2.2 g/ml, 2.3 g/ml, 2.5 g/ml, 2.7 g/ml, 3.0 g/ml or higher. The medium, excipient, or solution may or may not be sterile.

The medium, excipient, or solution may contain preservative agents to maintain the sample in an adequate state for subsequent diagnostics or manipulation, or to prevent coagulation. Said preservatives may include citrate, ethylene diamine tetraacetic acid, sodium azide, or thimerosol. The sample may be fixed prior to or during storage by any method known to the art such as using glutaraldehyde, formaldehyde, or methanol. The container may be any container suitable for storage and or transport of the biological sample including but not limited to: a cup, a cup with a lid, a tube, a sterile tube, a vacuum tube, a syringe, a bottle, a microscope slide, or any other suitable container. The container may or may not be sterile. In some cases, the sample may be stored in a commercial preparation suitable for storage of cells for subsequent cytological analysis such as but not limited to Cytyc ThinPrep, SurePath, or Monoprep.

The storage temperature may be explicitly defined or defined by a temperature range. The sample may be stored at room temperature or at reduced temperatures such as cold temperatures (e.g. between about 20° C. and about 0° C.), or freezing temperatures, including for example 0′C, −1° C., −2° C., −3° C., −4° C., −5° C., −6° C., −7° C., −8° C., −9° C., −10° C., −12° C., −14° C., −15° C., −16° C., −20° C., −22° C., −25° C., −28° C., −30° C., −35° C., −40° C., −45° C., −50° C., −60° C., −70° C., −80° C., −100° C., −120° C., −140° C., −180° C., −190° C., or about −200° C. The sample may be stored in any condition or environment that allows or achieves the desired. temperature condition. In some cases, the samples may be stored in a refrigerator, on ice or a frozen gel pack, in a freezer, in a cryogenic freezer, on dry ice, in liquid nitrogen, or in a vapor phase equilibrated with liquid nitrogen.

The sample container may be any container suitable for storage and or transport of the biological sample including but not limited to: a cup, a cup with a lid, a tube, a sterile tube, a vacuum tube, a syringe, a bottle, a microscope slide, or any other suitable container. The container may or may not be sterile.

3. Sample Conveyance and Transportation

Additionally contemplated in the current methods are methods of transporting a sample. Transport may involve moving or conveyance of a sample to or from a clinic, hospital, doctor's office, or other location to a second location. Upon transport the sample may be stored and/or analyzed by for example, cytological analysis or molecular profiling. In some embodiments some aspect of analysis, processing or profiling may begin or take place during transport. In some cases, the sample may be transported to a molecular profiling company in order to perform the analyses described herein. In other cases, the sample may be transported to a laboratory such as a laboratory authorized or otherwise capable of performing the methods described herein, such as a Clinical Laboratory improvement Amendments (CLIA) laboratory.

In some instances the subject may transport the sample. Transportation by an individual may include the individual appearing at a molecular profiling business or a designated sample receiving point and providing a sample. Providing of the sample may involve any of the techniques of sample acquisition described herein, or the sample may have already have been acquired and stored in a suitable container. In other cases the sample may be transported to a molecular profiling business using a courier service, the postal service, a shipping service, or any method capable of transporting the sample in a suitable manner.

In some cases, the sample may be provided to a molecular profiling business by a third party testing laboratory (e.g. a cytology lab). In other cases, the sample may be provided to a molecular profiling business by the subject's primary care physician, endocrinologist or other medical professional. The cost of transport may be billed to the individual, medical provider, or insurance provider. The molecular profiling business may begin analysis of the sample immediately upon receipt, or may store the sample in any manner described herein. The method of storage may or may not be the same as chosen prior to receipt of the sample by the molecular profiling business.

The sample may be transported in any medium or excipient including any medium or excipient provided herein suitable for storing the sample such as a cryopreservation medium or a liquid based cytology preparation. In some cases, the sample may be transported frozen refrigerated such as at any of the suitable sample storage temperatures provided herein.

Once the sample is received, the sample may be assayed using a variety of routine analyses known to the art such as cytological assays, and genomic analysis by a molecular profiling business, a representative or licensee thereof, a medical professional, researcher, or a third party laboratory or testing center (e.g. a cytology laboratory). Such tests may be indicative of cancer, the type of cancer, any other disease or condition, the presence of disease markers, or the absence of cancer, diseases, conditions, or disease markers. The tests may take the form of cytological examination including microscopic examination as described below. The tests may involve the use of one or more cytological stains. The biological material may be manipulated or prepared for the test prior to administration of the test by any suitable method known to the art for biological sample preparation. The specific assay performed may be determined by the molecular profiling company, the physician who ordered the test, or a third party such as a consulting medical professional, cytology laboratory, the subject from whom the sample derives, or an insurance provider. The specific assay may be chosen based on the likelihood of obtaining a definite diagnosis, the cost of the assay, the speed of the assay, or the suitability of the assay to the type of material provided.

4. Sample Integrity Tests

In some embodiments, concurrent with sample acquisition, sample storage or sample analysis the sample may be subjected to tests or examination that detail or reveal the integrity of the sample for use in the compositions or methods described herein. As a result of an integrity test a sample may be determined to be adequate or inadequate for further analysis.

In some embodiments sample integrity tests may pertain to the quality, integrity or adequacy of cells and or tissue in the sample. Metrics employed to determine quality, integrity or adequacy may involve but are not limited to cell number tests, cell viability tests, nuclear content tests, genetic content tests, biochemical assays, cell mass tests, cell volume tests, PCR tests, Q-PCR tests, RT-PCR tests, immunochemical analysis, histochemical analysis, microscopic analysis or visual analysis.

In certain aspects sample integrity may be ascertained by tests that measure nucleic acid content or integrity. Nucleic acid content tests may measure DNA content, RNA content or a some mixture of DNA or RNA. In some aspects nucleic acids are extracted or purified from other cellular components prior to a nucleic acid content test. In some embodiments nucleic acid specific dyes are used to assay nucleic acid integrity. In cases of nucleic acid extraction, spectrophotometric or electrophoretic methods may be used to assay nucleic acid integrity.

In yet other aspects, sample integrity may be ascertained by tests that measure protein content or integrity. Methods that measure protein content or integrity are well known to those skilled in the art. Such methods include but are not limited to ultraviolet absorbance reading (e.g. 280 nm absorbance readings), cell staining, protein staining or immunocytochemical methods. In some instances tests may be performed in parallel in intact samples or the samples may be divided and tests performed serially or in parallel.

Integrity tests may be performed on small subsets or aliquots of a sample or on the entirety of a sample.

C. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization, conditions are well known to those of skill in the art and reviewed in Sambrook et al. (2001) and WO 95/21944. Of particular interest in embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding, to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays can be used to detect differences between two samples. Specifically contemplated applications include identifying and/or quantifying differences between miRNA from a sample that is normal and from a sample that is not normal, between a cancerous condition and a non-cancerous condition, or between two differently treated samples. Also, miRNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. No. 5,384,261, incorporated herein by reference in its entirety. Although a planar array surface is used ii certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate (see U.S. Pat. Nos. 5,770,358, 5,789,162, 5,708,153, 6,040,193 and 5,800,992, each of which is hereby incorporated in its entirety). Arrays may be packaged in such a manner as to allow for diagnostics or other manipulation of an all inclusive device (see for example, U.S. Pat. Nos. 5,856,174 and 5,922,591, each incorporated in its entirety by reference). See also U.S. patent application Ser. No. 09/545,207, filed Apr. 7, 2000, which is incorporated by reference in its entirety for additional information concerning arrays, their manufacture, and their characteristics,

Particularly, arrays can be used to evaluate samples with respect to diseases or conditions that include, but are not limited to: benign thyroid conditions and malignant thyroid tumors, including mutation- and translocation-negative tumors.

Moreover, miRNAs can be evaluated with respect to the following diseases, conditions, and disorders: benign thyroid conditions and, malignant thyroid tumors, including mutation- and translocation-negative tumors; conventional follicular carcinomas (FC); oncocytic. follicular carcinomas (OFC); nodular hyperplasias (NH); follicular adenomas (FA); papillary carcinomas (PC). In addition, miRNAs can be evaluated with respect to thyroid tumors and the determination of whether a particular tumor is not generally malignant or aggressive. Also, miRNAs can be evaluated with respect to determining whether a particular tumor is generally considered malignant and/or aggressive.

It is specifically contemplated that the disclosed methods and compositions can be used to evaluate differences between stages of disease, such as between hyperplasia, neoplasia, pre-cancer and cancer, or between a primary tumor and a metastasized tumor, or between a lesion that is low risk (i.e., not generally malignant or aggressive) and a lesion that is high risk (i.e., generally considered malignant and/or aggressive).

E. Other Assays

In addition to the use of arrays and microarrays, it is contemplated that a number of different assays could be employed to analyze miRNAs, their activities, and their effects. Such assays include, but are not limited to, nucleic acid amplification, polymerase chain reaction, quantitative PCR, RT-PCR, in sire hybridization, Northern hybridization, hybridization protection assay (HPA), branched DNA (bDNA) assay, rolling circle amplification (RCA), single molecule hybridization detection, Invader assay, and/or Bridge Litigation Assay.

F. Evaluation of Expression Levels

A variety of different models can be employed to evaluate expression levels and/or other comparative values based on expression levels of miRNAs (or their precursors or targets). One model used in the Examples described below is a logistic regression model (see the Wikipedia entry on the World Wide Web at en.wikipedia, which is hereby incorporated by reference).

Start by computing the weighted sum of the DiffPair values:

z=β₀+β₁* Diff(miR₁ₐ,miR_{1b})+β₂*Diff(miR₂ₐ,miR_{2b})+ . . .

where the β₀ is the (Intercept) term identified in the spreadsheets, while the remaining βi are the weights corresponding to the various DiffPairs in the model in question. Once z is computed, the score p_{alignment} (which may be interpreted as predicted probability of malignancy) is calculated as

$${P}_{malignant} = \frac{1}{1 + exp ( - z )}$$

This functions to turn the number z, which may be any value from negative infinity to positive infinity, into a number between 0 and 1, with negative values for z becoming scores/probabilities of less than 50% and positive values for z becoming scores/probabilities of greater than 50%.

Other examples of models include but are not limited to Decision Tree, Linear Disciminant Analysis, Neural Network, Support Vector Machine, and k-Nearest Neighbor Classifier. A. person of ordinary skill in the art could use these different models to evaluate expression level data and comparative data involving expression levels of one or more miRs (or their precursors or their targets):

Models may take into account one or more diff pair values or they may also take into account differential expression of one or more miRNAs not specifically as part of a diff pair. A diagnostic score may be based on 1, 2, 3, 4, 5, 6, 7, 8 or more diff pair values (or any range derivable therein), but in some embodiments, it takes into account additionally or alternatively, 1, 2, 3, 4, 5, 6, 7; 8, 9, 10 or more miRNA expression levels (or any range derivable therein), wherein the miRNA expression level detectably differs between low risk and high risk lesions.

It will be understood by those of skill in the art that instead of a diff pair value, methods may involve a coefficient value that can be used in conjunction with the level of expression of a particular miRNA based on classifier analysis. Whereas the expression value associated with a diff pair is 1 times the expression value of the first miRNA of the pair summed with −1 times the expression value of the second miRNA of the diff pair, a more general classifier may be built with features composed of combinations of 2 or more miRNA expression values with coefficient values other than just 1 or −1.

Additionally, embodiments, may be based on a constrained logistic regression model. For illustrative purposes, consider a model built with three biomarker predictors A, B, and C. The classifier is specified by the weights w₀, w₁, w₂, and w₃: given a sample with expression value x₁ for marker A, x₂ for marker B, and x₃ for marker C, the model score may be computed as

$${P}_{malignant} = \frac{1}{1 + exp ( - z )} = \frac{1}{1 + exp ( - {w}_{0} - {w}_{1} {x}_{1} - {w}_{2} {x}_{2} - {w}_{3} {x}_{3} )}$$

$$where$$

$$\begin{matrix}z = {w}_{0} + {w}_{1} {x}_{1} + {w}_{2} {x}_{2} + {w}_{3} {x}_{3} \\ = {w}_{0} + {w}_{1} ( {x}_{1} - {x}_{2} ) + ( {w}_{1} + {w}_{2} ) ( {x}_{2} - {x}_{3} ) + ( {w}_{1} + {w}_{2} + {w}_{3} ) {x}_{3}\end{matrix}$$

Since a model may be constrained to satisfy w1+w2+w3=0, the last term m the above equation must vanish. But what's left expresses our model score in terms of the diff pairs Diff(A,B) (with expression value x1−x2) and Diff(B,C) (with expression value x2−x3). This argument can be extended in a straight-forward manner to apply to constrained logistic regression models with any number of predictors.

Alternatively, the converse may be done. A logistic regression model built using two DiffPairs, Diff(A,B) and Diff(C,D), with weights W₁ and W₂ can be equivalently described directly in terms of the underlying mils A, B, C, and D with weights w₁=W₁, w₂=−W₁, w₃=W₂, and w₄=−W₂ (in case of miR overlap, we just add together the relevant coefficients: e.g., if B=C, we would have w₁=W₁, w₂=−W₁+W₂, w₃=−W₂, with x₃ now describing the expression value of D). Because the weight of each diff pair appears twice in the resulting individual miRs, once with a positive sign and once with a negative sign, the sum of the resulting individual miR coefficients (the lower-case w₁'s) must be zero, satisfying our constraint condition.

### IV. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well as reagents for preparation of samples from thyroid samples. The kit may further include reagents for creating or synthesizing miRNA probes. Such kits may thus comprise, in suitable container means, an enzyme for labeling the miRNA by incorporating labeled nucleotides or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. Kits may also include one or more buffers, such as a reaction buffer, labeling buffer, washing buffer, or hybridization buffer, compounds for preparing the miRNA probes, and components for isolating miRNAs. Other kits may include components for making a nucleic acid array comprising miRNAs, and this, may include, for example, a solid support.

Kits for implementing methods described herein are specifically contemplated. In. some embodiments, there are kits for preparing miRNAs for multi-labeling and kits for preparing miRNA probes and/or miRNA arrays. In such embodiments, kits comprise, in suitable container means, 1, 2, 3, 4, 5, 6, 7, 8, 9, 10, 11, 12 or more of the following: 1) poly(A) polymerase; 2) unmodified nucleotides (G, A, T, C, and/or U); 3) a modified nucleotide (labeled or unlabeled); 4) poly(A) polymerase buffer, 5) at least one microfilter; 6) label that can be attached to a nucleotide; 7) at least one miRNA probe; 8) reaction buffer; 9) a miRNA array or components for making such an array; 10) acetic acid; 11) alcohol; 12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits include an array containing miRNA. probes, as described in the application. An array may have probes corresponding to all known miRNAs of an organism or a particular tissue or organ in particular conditions, or to a subset of such probes. The subset of probes on arrays may be or include those identified. as relevant to a particular diagnostic, therapeutic, or prognostic application. For example, the array may contain one or more probes that are indicative or suggestive of 1) a disease or condition (thyroid cancer), 2) susceptibility or resistance to a particular drug or treatment; 3) susceptibility to toxicity from a drug or substance; 4) the stage of development or severity of a disease or condition (prognosis); and 5) genetic predisposition to a disease or condition.

For any kit embodiment, including an array, there can be nucleic acid molecules that contain or can be used to amplify a sequence that is a variant of, identical to, or complementary to all or part of any of the sequences disclosed herein. In certain embodiments, a kit or array can contain one or more probes for the miRNAs identified by sequences disclosed herein. Any nucleic acid discussed above may be implemented as part of a kit.

Components of kits may be packaged either in aqueous media or in lyophilized form: The container means of the kits will-generally include at least one vial, test tube, flask, bottle, syringe, or other container means, into which a component may be placed, and preferably, suitably aliquoted. Where there is more than one component in the kit (e.g., labeling reagent and label may be packaged together), the kit also will generally contain a second, third, or other additional container into which the additional components may be separately placed. However, various combinations of components may be comprised in a vial. The kits also may include a means for containing the nucleic acids, and any other reagent containers in close confinement for commercial sale. Such containers may include injection or blow molded plastic containers into which the desired vials are retained.

When the components of a kit are provided in one and/or more liquid solutions, the liquid solution may be an aqueous solution, with a sterile aqueous solution being particularly preferred.

However, the components of a kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg, or at least or at most those amounts, of dried dye are provided in kits. The dye may then be resuspended in any suitable solvent, such as DMSO.

The container means will generally include at least one vial, test tube, flask, bottle, syringe and/or other container means, into which the nucleic acid formulations are placed, for example, suitably allocated. Kits may also comprise a second container means for containing a sterile, pharmaceutically acceptable buffer and/or other diluent.

Kits may include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit may also include instructions for employing the kit components as well the use of any other reagent not included in the kit. Instructions may include variations that can be implemented.

Kits may also include one or more of the following: control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits. Such kits, however, are not limited to the particular items identified above and may include any reagent used for the manipulation or characterization of miRNA.

### V. EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art will, in light of the present disclosure, appreciate that many changes can be made in the specific embodiments that are disclosed and still obtain a like or similar result without departing from the spirit and scope of the invention.

### Example 1

The goal was to identify classifiers than can accurately distinguish benign thyroid conditions from malignant thyroid tumors, including mutation- and translocation-negative tumors, in preoperative thyroid FNAs. Papillary thyroid carcinomas are characterized, in particular, by the up-regulation of three miRNAs, miR-146b, miR-221, and miR-222, which is even more pronounced in BRAF V600E-positive tumors.

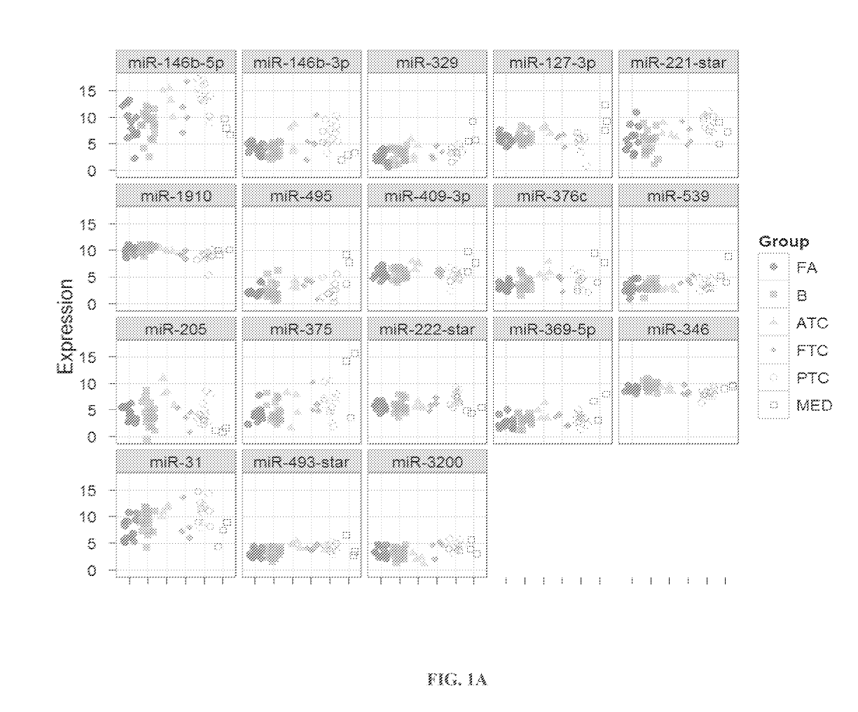

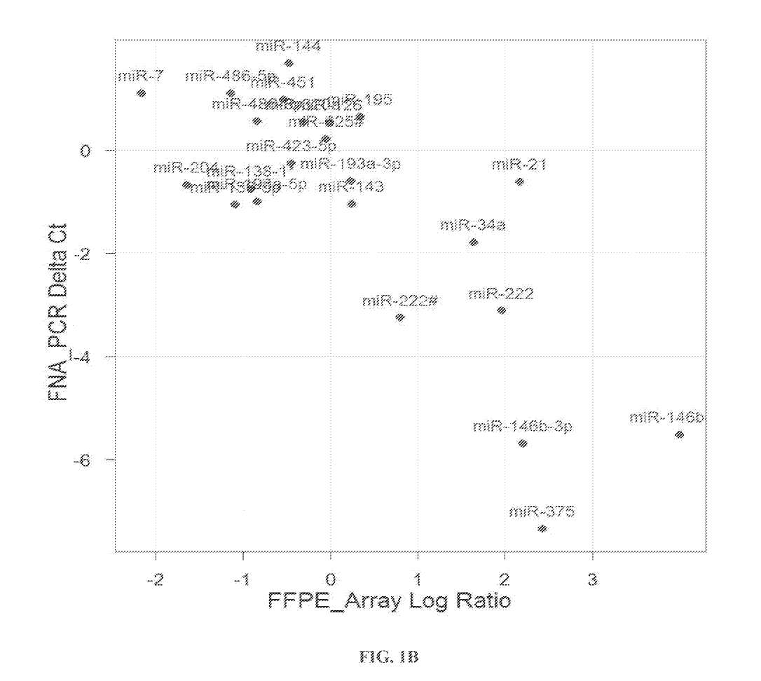

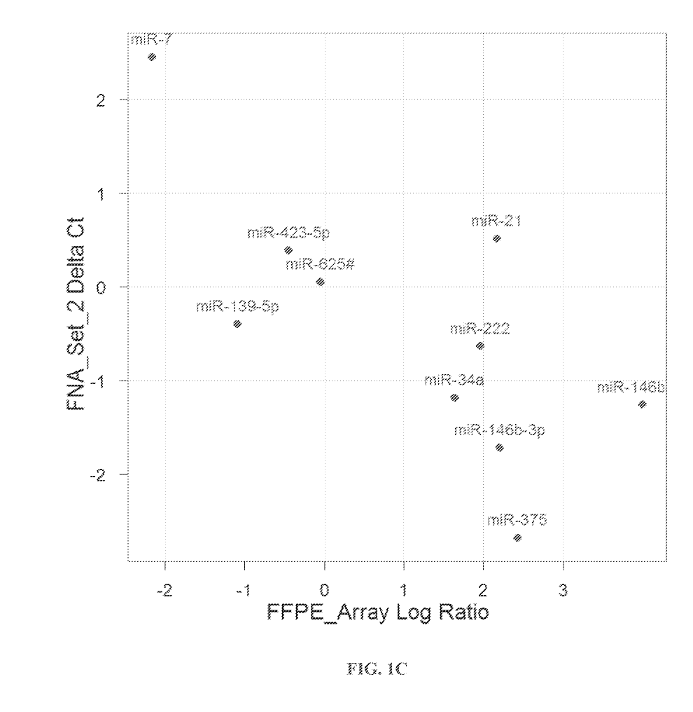

The classifier consists of a mathematical algorithm to predict probability of malignancy from the qRT-PCR measurements of the expression levels of the following microRNA species in FNA samples: miR-7, miR-21, miR31, miR-34a, miR-138-1, miR-139-5p, miR-141, miR-145, miR-146b-3p, miR-146b-5p, miR-155, miR-183, miR-197, miR-222, miR-224, miR-375, and miR-425. These microRNAs were identified through statistical differential expression analysis of fresh frozen and FFPE samples comparing various benign and malignant sample types, FIG. 1 illustrates the differentially expressed miRNAs identified from FFPE Samples. FIG. 1A reflects 18 differentially expressed miRs identified by LIMMA ANOVA analysis of Affymetrix miRNA array data (all significant at p<0.05 after Benjamini-Hochberg FDR adjustment). This analysis, in conjunction with pair-wise comparisons between different sample subgroups, led to the identification of a set of miRNAs for qRT-PCR Analysis. Good correlation was observed for benign-vs-malignant fold changes (shown as log-ratios for array and delta-Ct for PCR) between the array data and the PCR data for selected miRNA chosen for assay in FNA set 1 (B) and FNA set 2 (C), B=benign conditions other than FA.

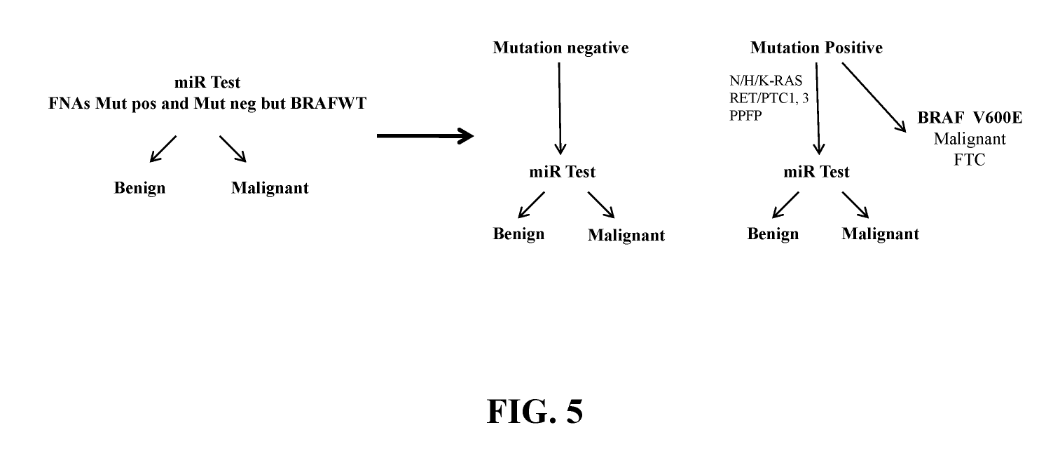

This was followed by a second round of feature selection conducted. by L1-constrained logistic regression modeling. Prior indication of differential expression of microRNA species in the literature was considered as an additional factor for those miRs which were found to be strongly predictive of malignancy status in regression modeling. FIG. 5 illustrates the approach for modeling the distinction between benign and malignant FNA samples used.

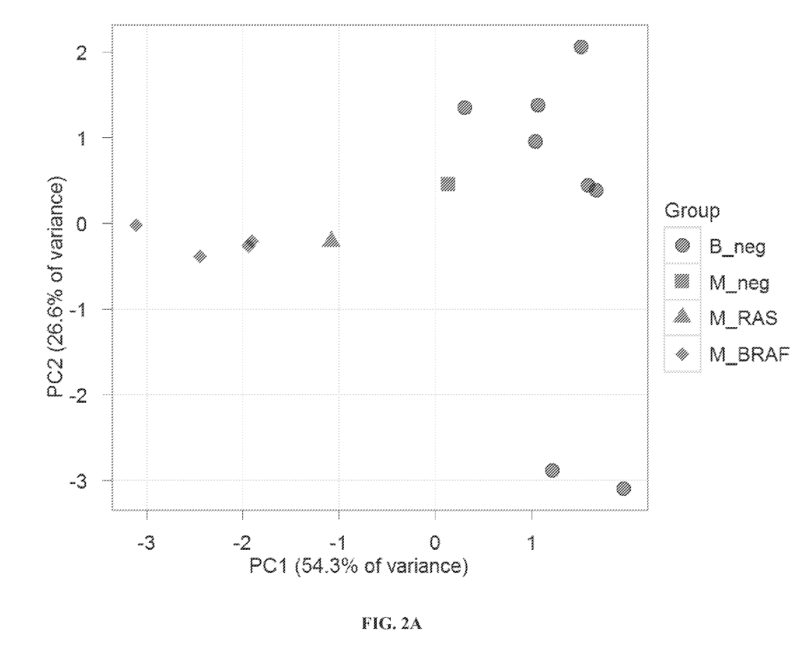

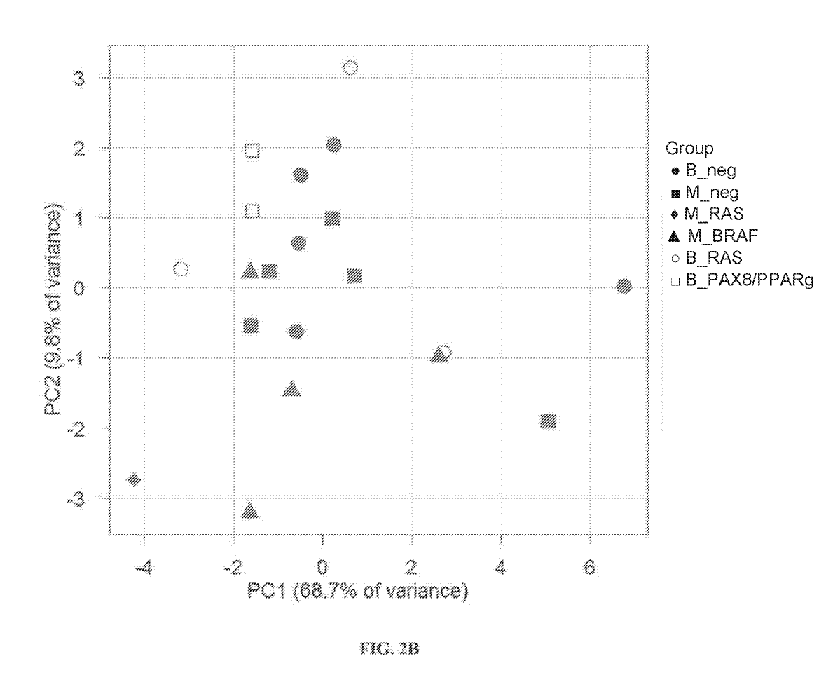

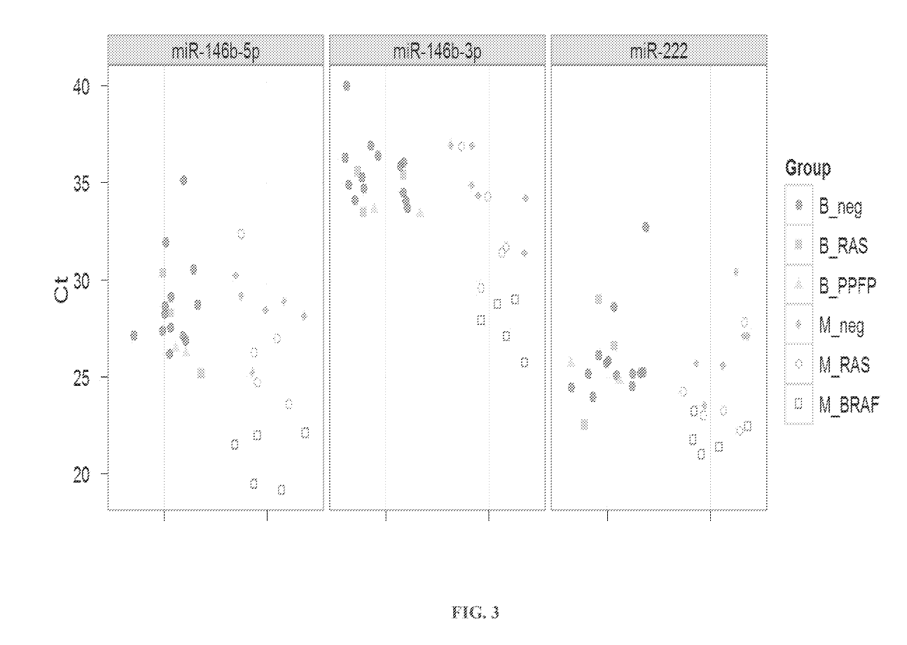

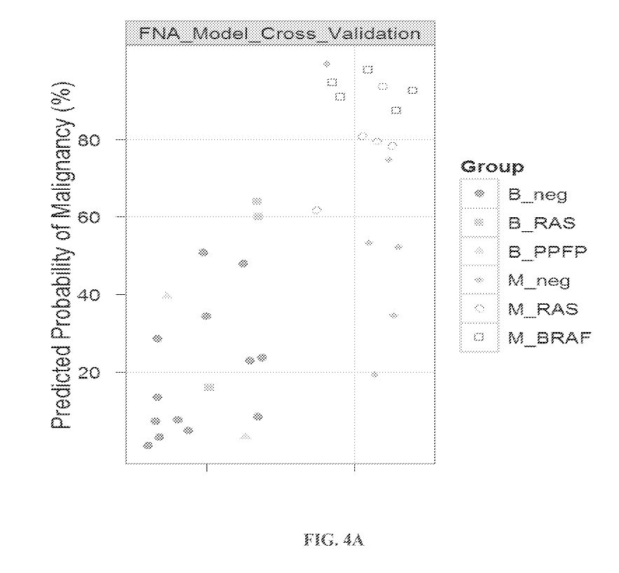

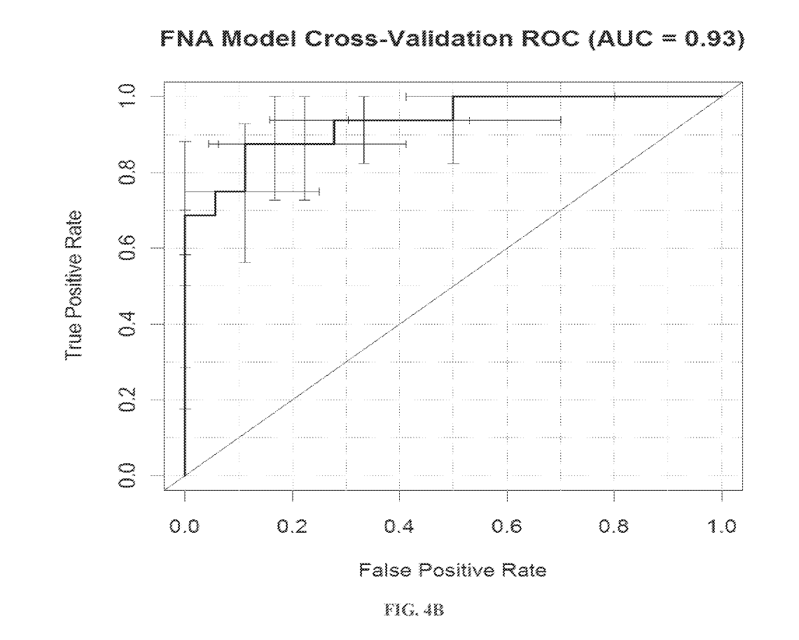

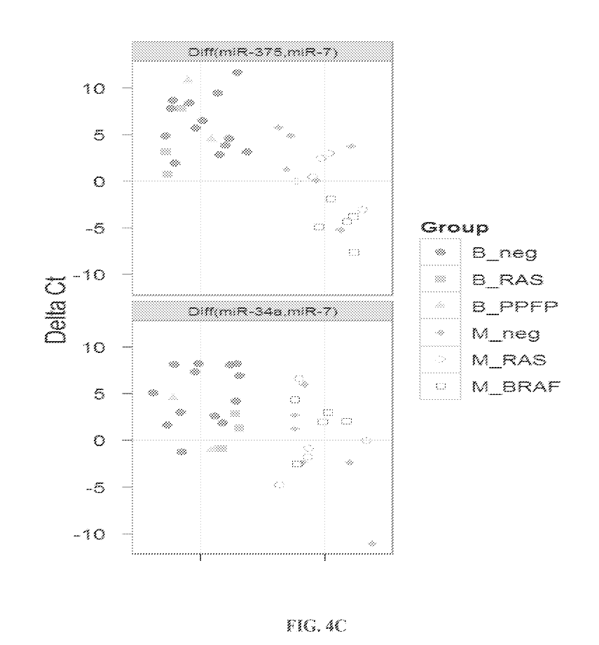

The first analysis revealed BRAF V600E mutation to be a confounding factor for the development of classifiers. FIG. 2 illustrates miRNA expression in FNA specimens correlated with mutational status. Principal Components Analysis (PCA) applied to qRT-PCR data (raw Ct values) derived from FIG. 2A FNA set 1 and FIG. 2B FNA set 2. Both sets show clear separation of BRAF-mutation positive samples from benign, less separation between mutation-negative malignant samples and benign samples. B_neg=benign, mutation-negative; M_BRAF=malignant, V600E mutation-positive; M_RAS=malignant, RAS mutation-positive; M_neg=malignant, mutation-negative. A preliminary logistic regression classifier for FNA samples is shown in FIG. 3, miR-146b and miR-222 show strongly differential expression between BRAF WT and mutant populations, but are much less useful for distinguishing mutation negative malignant from benign, specimens. FIG. 4 illustrates that the strongest miRNA signals in FNAs correlate with BRAF status. Logistic model fit to merged FNA set (using 11 miRNA species assayed on both FNA sets). Model was fit using λ₁ penalized regression to DiffPaired. data. The performance of this model was assessed using 5-fold cross-validation, with resulting malignancy predictions shown in FIG. 4A and resulting ROC curve shown in FIG. 4B. Four DiffPairs received non-zero weights in final model, with the top two DiffPairs (values shown in FIG. 4C) having >5-fold larger weights than the bottom two.

As BRAF V600E is highly specific, of malignancy, the inventors focused subsequent analyses on FNA specimens that were BRAF wild-type. This allowed the inventors to show that classifiers based on miRs-375 and miR-7, among others, show promise for identification of BRAF wild-type malignancies in FNA biopsies.

### Materials and Methods

To identify candidate miRNAs, two independent sets of thyroid samples were procured that corresponded to a variety of benign and malignant conditions. The two sample sets consisted. of 46 fresh frozen biopsies (data not shown) and 55 formalin-fixed paraffin-embedded (FEPE) samples, respectively. Total RNA was isolated from FFPE samples using the RecoverAll kit (Ambion), miRNA expression profiles were established on Affymetrix GeneChip miRNA 2.0 Arrays (Affymetrix) that target 1,105 human miRNAs from the Sanger database V15 (FFPE specimens). The samples were divided into benign and malignant groups for analysis, according to histology.

FNAs were obtained from an on-going multi-center study. Total nucleic acids were isolated from the FNAs using a proprietary method developed by Asuragen. qRT-PCR reactions were performed using specific TaqMan miRNAs assays (Applied Biosystems), as selected from the array data analysis.

Affymetrix GeneChip miRNA Service Methods Summary

Sample and Array Processing.

Samples for miRNA profiling studies were processed by Asuragen Services (Austin, TX), according to the company's standard operating procedures. Following incoming sample quality control (QC) assessment, the 3′ ends of RNA molecules in total RNA samples were labeled with biotin according to the company's standard protocol. Labeled RNA (100 ng total RNA per sample) was purified and hybridized to Affymetrix GeneChip® miRNA Arrays (Affymetrix, Santa Clara, Calif.). Hybridization, washing, staining, imaging, and signal extraction were performed according to Affymetrix-recommended procedures. Arrays were scanned on an. Affymetrix GeneChip® Scanner 3000 7G.

Normalization and Summarization.

Probe level summaries were extracted using median polish and Variance Stabilization Normalization (VSN) was applied to the array data. This is a global normalization approach that models the quadratic relationship between the variance of microarray data and the signal intensity, and transforms the data such that the variance is roughly constant. The post-normalized data scale is reported as generalized log 2 data. As the distribution of microarray data tends to approximately follow a normal distribution pattern after log transformation, this transformation helps to render normalized data amendable to classical statistical treatments, including t-tests and ANOVA.

Statistical Hypothesis Testing.

The signal processing implemented for the Affymetrix miRNA array is a multi-step process involving probe specific signal detection calls, background correction, and array scaling. For each probe, the contribution of signal due to background Was estimated and removed by the feature extraction software as part of the data file output. Similarly, detection calls were based on the feature extraction software. Arrays within a specific analysis experiment were normalized together according to the VSN scaling method.

The signal processing implemented for the Affymetrix miRNA array is a multi-step process involving probe specific signal detection calls, background correction, and global normalization. For each probe, the contribution of signal due to background was estimated and removed by the feature extraction software as part of the data file output. Similarly, detection calls were based on the feature extraction software. Arrays within a specific analysis experiment were normalized together according to the VSN method described by Bolstad B et al [1].

The signal processing implemented for the Affymetrix miRNA array is a multi-step process involving probe specific signal detection calls, background estimate and correction, constant variance stabilization and log 2 transformation. For each probe, an estimated background value is subtracted that is derived from the median signal of a set of G-C-matched anti-genomic controls. Detection calls were based on a Wilcoxon rank-sum test of the miRNA probe signal compared to the distribution of signals from GC-content matched anti-genomic probes.

The signal processing implemented for the Affymetrix miRNA array is a multi-step process involving probe specific signal detection calls, background correction, and array scaling. For each probe, the contribution of signal due to background was estimated and removed by the feature extraction software as part of the data file output. Similarly, detection calls were based on the feature extraction software. Arrays within a specific analysis experiment were normalized together according to the VSN scaling method to the 1^{th} percentile.

For statistical hypothesis testing, both equal variance two-sample t-Tests and one-way ANOVA were applied using empirical Bayes methods. (as implemented by the Limma R package) to shrink individual miRNA variances towards a common value. This approach of borrowing information from the entire set of miRNAs increases the stability and power of the statistical test. All p-values are adjusted for false discovery rate using the method described by Benjamini & Hochberg (Benjamini & Hochberg (1995)) to account for testing all human mature miRNAs for differential expression.

Sample Information.

A total of 55 formalin-fixed paraffin-embedded (FFPE) samples from 31 benign and 24 malignant neoplasms were purchased from Asterand (Asterand Plc., Detroit, Mich., USA). The benign samples corresponded to 11 hyperplastic nodules (HN), 10 follicular adenomas (FA), 2 oncocytic follicular adenoma (OFA), 4 multi nodular goiter (MNG), and 4 Hashimoto's thyroiditis (HASH). The malignant samples corresponded to 8 papillary carcinomas (PTC), 5 follicular variant of papillary carcinomas (FVPTC), 2 follicular carcinomas (FTC), 2 oncocytic variant of follicular carcinoma (OFTC), 3 anaplastic carcinoma (ATC), and 4 medullary carcinomas (MED). Information regarding these samples is provided in Table 2. Total RNA was isolated from each sample, using the RecoverAll RNA Isolation Kit (Ambion; Austin, Tex., USA). Total RNA was quantified using a NanoDrop® ND-1000 spectrophotometer (NanoDrop Technologies Inc, Wilmington, Del., USA).

TABLE 2
Patient information and histopathological data for the thyroid FFPE samples.

| | | Age at | | | Excision | AJCC/ | AJCC/UICC |
|---|---|---|---|---|---|---|---|
| Sample ID | Histopathology | Excision | Sex | Ethnicity | Year | UICC Stage | Stage Group |
| AF 91 | ATC | 62 | Male | Caucasian | 2000 | T4aNXMX | IVA |
| AF 101 | ATC | 76 | Female | Caucasian | 2004 | T4aN1aMX | IVA |
| AF 102 | ATC | 49 | Male | Caucasian | 2005 | T4aN1bMX | IVA |
| AF62 | ATC | N/A | N/A | N/A | N/A | N/A | N/A |
| AF60 | FA | 38 | Male | Caucasian | 2004 | | |
| AF63 | FA | 65 | Female | Caucasian | 1995 | | |
| AF71 | FA | 47 | Female | Caucasian | 1995 | T2NXMX | II |
| AF76 | FA | 69 | Male | Caucasian | 2004 | | |
| AF77 | FA | 71 | Female | Caucasian | 2004 | T2NXM0 | II |
| AF81 | FA | 30 | Female | Caucasian | 2001 | | |
| AF82 | FA | 66 | Female | Caucasian | 2001 | | |
| AF83 | FA | 35 | Female | Caucasian | 2001 | | |
| AF88 | FA | 68 | Female | Caucasian | 2006 | T1NXMX | I |
| AF113 | FA | 63 | Female | Asian/ | 2001 | | |
| | | | | Pacific Islander | | | |
| AF64 | FTC | 47 | Female | Caucasian | 1995 | T2NXMX | II |
| AF 111 | FTC | 56 | Female | Caucasian | 2008 | T2N0MX | II |
| AF47 | FVPTC | 80 | Female | Caucasian | 2009 | T2N0M0 | II |
| AF61 | FVPTC | 67 | Male | Caucasian | 1993 | T3NXMX | III |
| AF67 | FVPTC | 42 | Female | Caucasian | 1996 | T3NXMX | III |
| AF72 | FVPTC | 25 | Female | Caucasian | 1993 | T1bN1MX | I |
| AF 107 | FVPTC | 63 | Male | Caucasian | 2009 | T2N0MX | II |
| AF50 | HASH | 41 | Female | Caucasian | 2010 | T1aNXMO | I |
| AF54 | HASH | 62 | Female | Caucasian | 1995 | T3NXMX | III |
| AF74 | HASH | 36 | Female | Caucasian | 1995 | | |
| AF75 | HASH | 45 | Female | Asian/ | 1995 | T1aN0MX | I |
| | | | | Pacific Islander | | | |
| AF70 | MED | 62 | Female | Caucasian | 1996 | T3NXMX | III |
| AF 99 | MED | 37 | Male | Native American/ | 2003 | T3N1bMX | IVA |
| | | | | Alaskan | | | |
| AF 104 | MED | 60 | Female | Caucasian | 2007 | T2N1bMX | IVA |
| AF 90 | MNG | 48 | Male | Caucasian | 2000 | | |
| AF 95 | MNG | 47 | Female | Caucasian | 2001 | | |
| AF 96 | MNG | 67 | Female | Caucasian | 2002 | | |
| AF 98 | MNG | 60 | Female | Caucasian | 2003 | | |
| AF48 | NH | 22 | Female | Caucasian | 2009 | T3N1M0 | I |
| AF49 | NH | 64 | Female | Caucasian | 2009 | | |
| AF53 | NH | 70 | Male | Caucasian | 2009 | | |
| AF55 | NH | 50 | Female | Caucasian | 2008 | T1NXM0 | I |
| AF56 | NH | 61 | Female | Caucasian | 2008 | TXNXM0 | |
| AF57 | NH | 80 | Female | Caucasian | 2008 | T2N1M1 | IV |
| AF58 | NH | 46 | Female | Caucasian | | | |
| AF59 | NH | 66 | Male | Caucasian | 2004 | | |
| AF66 | NH | 44 | Female | Caucasian | 1996 | T1bN1MX | III |
| AF78 | NH | 82 | Male | Caucasian | 2004 | | |
| AF87 | NH | 57 | Female | Caucasian | 2006 | T1NXMX | I |
| AF 108 | OFA | 56 | Female | Caucasian | 1997 | | |
| AF110 | OFA | 38 | Male | Caucasian | 2001 | | |
| AF69 | OFTC | 46 | Female | Caucasian | 1996 | T3NXMX | III |
| AF 92 | OFTC | 86 | Female | | 2000 | T2N1MX | III |
| AF52 | PTC | 38 | Female | Caucasian | 2009 | T2NXMX | I |
| AF85 | PTC | 45 | Female | Caucasian | 2006 | T2N0M0 | II |
| AF86 | PTC | 77 | Female | Caucasian | 2006 | T1NXMX | I |
| AF89 | PTC | 46 | Female | Caucasian | 2006 | T1NXMO | I |
| AF 100 | PTC | 55 | Female | Caucasian | 2003 | T3N1bMX | IVA |
| AF 103 | PTC | N/A | N/A | N/A | N/A | N/A | N/A |
| AF 105 | PTC | 61 | Male | Caucasian | 2008 | T3N1bM0 | IVA |
| AF 109 | PTC | 79 | Male | Caucasian | 1999 | T4aN1MX | IVA |

Anaplastic cancer (ATC);
Follicular Adenoma (FA);
oncocytic FA (OFA);
Follicular carcinoma (FTC);
oncocytic FTC (OFTC);
Papillary carcinoma (PTC);
Follicular variant of PTC (FVPTC);
medullary cancer (MED);
Hashimoto's thyroiditis (HASH);
multinodular goiter (MNG);
hyperplastic nodules (HN)

Two sets of fine needle aspiration (FNA) biopsies were used, each of these sets containing both benign and malignant FNA samples, as determined by cytology. Malignancy was confirmed by histology after resection of the lesion (Tables 3A and 3B). Molecular testing was performed using Asuragen miRInform™ Thyroid mutation panel assay.

TABLE 3A
Patient information and pathological reports on the first set (Set1) of FNA
samples used as a training set for the development of the miRNA classifier

| | | | | | Histopathology | |
|---|---|---|---|---|---|---|
| Sample | | | | | report after | Molecular |
| ID | Age | Gender | Cytology | Location | surgery | testing results |
| FNA1 | 55 | M | Benign | right | FA | Neg |
| FNA2 | 24 | M | Benign | right middle | | Neg |
| FNA3 | 53 | F | Benign | right lower | | Neg |
| FNA4 | 27 | F | Benign | right | | Neg |
| FNA5 | 28 | F | Benign | left | | Neg |
| FNA6 | 58 | F | Benign | Left | | Neg |
| FNA7 | 48 | F | Benign | left upper | | Neg |
| FNA8 | 81 | F | Benign | left lower | | Neg |
| FNA9 | 32 | F | Positive Papillary | right middle | PTC | BRAF V600E |
| | | | Carcinoma | | | |
| FNA10 | 44 | F | Positive Papillary | left lower | PFVPTC | NRAS Q61R |
| | | | Carcinoma | | | |
| FNA11 | 66 | F | Positive Papillary | right | FVPTC | Neg |
| | | | Carcinoma | | | |
| FNA12 | 46 | F | Positive Papillary | left middle | FVPTC | BRAF V600E |
| | | | Carcinoma | | | |
| FNA13 | 22 | F | Positive Papillary | left middle | FVPTC | BRAF V600E |
| | | | Carcinoma | | | |
| FNA14 | 43 | M | Positive Papillary | left lower | FVPTC | BRAF V600E |
| | | | Carcinoma | | | |

TABLE 3B
pathological reports and mutation status on the FNAs used as the second set
(Set 2) of FNA samples used for the development of a miRNA classifier

| | | | |
|---|---|---|---|
| Sample | | SURGERY/ | |
| ID | CYTOLOGY REPORT | HISTOLOGY | Mutational status |

Benign lesions Mutation negative

| | | | |
|---|---|---|---|
| FNA15 | HASH | N/A | Neg |
| FNA16 | Benign | N/A | Neg |
| FNA17 | Benign | N/A | Neg |
| FNA18 | Benign | N/A | Neg |
| FNA19 | Benign | N/A | Neg |

Benign lesions Mutation-positive

| | | | |
|---|---|---|---|
| FNA20 | Benign | N/A | KRAS7 G12V |
| FNA21 | Benign | N/A | PPFP |
| FNA22 | AUS/FLUS | N/A | HRAS G12V |
| FNA23 | Benign | N/A | NRAS Q61K |
| FNA24 | FLUS/AUS | N/A | PAX8/PPARg |

PTC mutation negative

| | | | |
|---|---|---|---|
| FNA25 | FLUS/AUS | FVPTC | Neg |
| FNA26 | FLUS/AUS | FVPTC | neg |
| FNA27 | FLUS/AUS | FVPTC | neg |
| FNA28 | FLUS/AUS | PTC | Neg |
| FNA29 | FLUS/AUS | PTC | Neg |

PTC mutation-positive

| | | | |
|---|---|---|---|
| FNA30 | Malignant | FVPTC | NRAS Q61R |
| FNA31 | Malignant | FVPTC | NRAS Q61R |
| FNA32 | Suspicious for malignancy | FVPTC | HRAS Q61R |
| FNA33 | Suspicious for neoplasm | FVPTC | NRAS Q61R |
| FNA34 | Suspicious for malignancy | FVPTC | HRAS Q61R |
| FNA35 | PTC | PTC | BRAF V600E |

FNA specimens, corresponding to an entire extra-pass, were collected according to the institutional IRB-approved protocol as a part of standard clinical care, and all samples were de-identified. Upon collection, FNAs were placed in 1 mL of Asuragen's RNARetain™ nucleic acid stabilization solution and shipped to Asuragen at room temperature for molecular testing. Total RNA was extracted using a modified mirVana PARIS™ procedure and proprietary Asuragen protocols optimized for recovery of nucleic acids from RNARetain. Concentration and purity of RNA were measured using the NanoDrop ND-1000 Spectrophotometer (NanoDrop Technologies Inc., Wilmington, Del., USA).

### Sample Description

| | | No. of | | | |
|---|---|---|---|---|---|
| FFPE | Category | Samples | Histopathology | | |
| 55 | Benign | 31 | HN (11); FA (10); OFA (2); MNG (4); HASH (4) | | |
| | Malignant | 24 | PTC (8); PVPTC (5); FTC (2); OFTC (2); MED (3); ATC (4) | | |
| | | No. of | | | |
| FNAs | Category | Samples | Cytology | Surgery/Histology | Mutational status* |
| Set 1 = 14 | Benign (8) | 8 | Non malignant cells | FA (1) | Neg |
| | Malignant (6) | 6 | Malignant | FVPTC (4), PTC (2) | BRAF V600E (4) NRAS Q61R (1) |
| Set 2 = 20 | Benign Mut neg (5) | 4 | Non malignant cells | N/A | Neg |
| | | 1 | Hashimoto | N/A | Neg |
| | Begnign Mut pos (5) | 3 | Non malignant cells | N/A | KRAS G12V (1) NRAS Q6IK (1) |
| | | | | | PAX8/PPARg (1) |
| | | 2 | AUS/FLUS | N/A | HRAS G12V (1) PAX8PPARg (1) |
| | Malignant Mut neg (5) | 5 | AUS/FLUS | PTC (2) FVPTC (3) | Neg |
| | Malignant Mut pos (5) | 2 | Malignant | PTC (2) | NRAS Q61R (2) |
| | | 2 | Susp for malignancy | FVPTC (2) | HRAS Q61R (2) |
| | | 1 | Suspfor neoplasm | FVPTC (1) | NRAS Q51R (1) |

*The mutational status of these samples was established using Asuragen's Luminex-based Research Assay.
Benign lesions: hyperplastic nodule (HN); Follicular adenoma (FA); oncocytic follicular adenoma (OFA); multinodular goiter (MNG); Hashimoto's thyroiditis (HASH);
Malignant lesions: papillary carcinoma (PTC); follicular variant of papillary carcinoma (FVPTC); follicular carcinoma (FTC); oncocytic follicular carcinoma (OFTC); medullary cancer (MED); anaplastic carcinoma (ATC)

### Example 2

### miRNAs can Distinguish Benign and Malignant Thyroid Neoplasms

A total of 55 FFPE samples from from 31 benign and 24 malignant neoplasms were profiled using the Affymetrix GeneChip® miRNA Arrays V2 in Asuragen Services and then analyzed by Asuragen's bioinformatics group (Asuragen). The Affymetrix miRNA arrays contain probes for more than 1,000 human miRNAs from Sanger miRBase VII.0 (available on the world wide web at asuragen.com/Services/services). A total of 200 ng of total RNA corresponding to each of the 55 samples was labeled as described in. the materials and methods. The samples were separated in 2 different groups for analysis, corresponding to Benign (hyperplastic nodules (HN; n=11), follicular adenoma (FA; n=10), oncocytic follicular adenoma (OFA; n=2), multi nodular goiter (MNG; n=4), and Hashimoto's thyroiditis (HASH; n=4) and Malignant (papillary carcinomas (PTC; n=8), follicular variant of papillary carcinomas (FVPTC; n=5), follicular carcinomas (FTC; n=2), oncocytic variant of follicular carcinoma (OFTC; n=2), anaplastic carcinoma (ATC; n=4), and medullary carcinomas (MED; n=4). The average levels of expression of each miRNA within each group of samples are shown in Tables 4A-D.

TABLE 4A
normalized array data for the 55 benign and malignant FFPE samples. Hyperplastic
nodule (HN); follicular adenoma (FA); oncocytic FA (OFA), multinodular goiter (MNG).

| | Average Expression | | | |
|---|---|---|---|---|
| miRNA | HN (Mean) | FA (Mean) | OFA (Mean) | MNG (Mean) |
| has-let-7a_st | 15.33134637 | 14.24099186 | 11.7936842 | 13.4739347 |
| has-let-7a-star_st | 2.110895451 | 1.791791809 | 1.248074076 | 1.173824172 |
| has-let-7a-2-star_st | 4.860529413 | 4.586453364 | 5.269724619 | 4.937201273 |
| has-let-7b_st | 14.69413715 | 13.25913718 | 10.57489326 | 12.14642872 |
| has-let-7b-star_st | 6.792844262 | 7.117992036 | 7.662555232 | 7.530672855 |
| has-let-7c_st | 15.23062851 | 13.88133156 | 11.35091982 | 12.99181533 |
| has-let-7c-star_st | 4.171332397 | 3.806653704 | 4.425062721 | 4.218655511 |
| hsa-let-7d_st | 14.76346582 | 13.81262879 | 11.64571066 | 12.62751338 |
| hsa-let-7d-star_st | 8.53187748 | 8.098607654 | 7.766016534 | 8.079830029 |
| has-let-7e_st | 13.09541233 | 12.28617016 | 10.45250531 | 11.10540492 |
| has-let-7e-star_st | 4.215115082 | 4.609380325 | 4.55332673 | 4.058074559 |
| hsa-let-7f_st | 14.59342893 | 13.91129749 | 11.51510071 | 12.76865153 |
| hsa-let-7f-1-star_st | 3.530423518 | 4.395882486 | 5.171252468 | 4.777049014 |
| hsa-let-7f-2-star_st | 1.650535016 | 1.454440181 | 2.457326428 | 1.573903791 |
| hsa-miR-15a_st | 10.06262181 | 10.33242942 | 6.089214326 | 7.032466977 |
| hsa-miR-15a-star_st | 3.374005266 | 3.850024561 | 4.930377243 | 4.45707072 |
| hsa-miR-16_st | 13.65491454 | 13.05403302 | 9.753821444 | 11.81436974 |
| hsa-miR-16-1-star_st | 2.884220775 | 3.157135553 | 3.116583089 | 2.04270118 |
| hsa-miR-17_st | 10.3925651 | 9.511397276 | 6.805043509 | 7.53169487 |
| hsa-miR-17-star_st | 4.982267167 | 6.18280036 | 4.656587948 | 3.927135405 |
| hsa-miR-18a_st | 4.575647081 | 4.114891284 | 3.099195998 | 2.671173077 |
| hsa-miR-18a-star_st | 4.768566013 | 5.071700097 | 5.116458511 | 4.069444557 |
| hsa-miR-19a-star_st | 0.951157914 | 0.390178671 | 0.159125319 | 0.740013802 |
| hsa-miR-19a_st | 4.165326791 | 4.720638613 | 1.708514281 | 2.456917344 |
| hsa-miR-19b-1-star_st | 2.486422955 | 2.619038729 | 2.290072306 | 2.393443376 |
| hsa-miR-19b_st | 9.547185652 | 9.231137075 | 5.898956336 | 7.370312978 |
| hsa-miR-19b-2-star_st | 0.814341412 | 1.154489192 | 0.393980058 | 1.303815242 |
| hsa-miR-20a_st | 9.756100841 | 9.082626556 | 6.966591876 | 6.063871985 |
| hsa-miR-20a-star_st | 5.973104762 | 5.932765668 | 7.793689194 | 6.583224777 |
| hsa-miR-21_st | 11.01084597 | 10.74976677 | 7.569426583 | 8.956136328 |
| hsa-miR-21-star_st | 5.608680329 | 5.617396845 | 5.793445199 | 6.336205876 |
| hsa-miR-22-star_st | 7.067117077 | 7.346201177 | 4.991743417 | 5.122410063 |
| hsa-miR-22_st | 12.41582277 | 11.57063023 | 10.08266085 | 10.1423768 |
| hsa-miR-23a-star_st | 10.49987149 | 10.52086187 | 11.25819113 | 11.31269572 |
| hsa-miR-23a_st | 14.87227126 | 13.69208321 | 11.11631748 | 12.79184378 |
| hsa-miR-24-1-star_st | 2.959612098 | 3.542333549 | 0.864472389 | 1.395382794 |
| hsa-miR-24_st | 13.72162874 | 12.71103737 | 10.21179484 | 11.31904231 |
| hsa-miR-24-2-star_st | 4.835122145 | 5.082053254 | 3.962836564 | 2.853893061 |
| hsa-miR-25-star_st | 12.29983445 | 12.10747545 | 12.6415432 | 13.06917051 |
| hsa-miR-25_st | 10.93305799 | 10.19648078 | 7.313209378 | 9.406423011 |
| hsa-miR-26a_st | 15.44341098 | 14.52683697 | 12.1348761 | 13.63241759 |
| hsa-miR-26a-1-star_st | 1.682603238 | 1.846078022 | 2.928478151 | 2.2429861 |
| hsa-miR-26b_st | 11.82468346 | 11.68895799 | 8.366104012 | 10.13776415 |
| hsa-miR-26b-star_st | 5.276948079 | 5.275218808 | 3.006360752 | 3.59024221 |
| hsa-miR-27a-star_st | 5.23209868 | 4.753705437 | 4.671827788 | 4.703555096 |
| hsa-miR-27a_st | 12.12234929 | 11.53552561 | 6.637891757 | 9.664558579 |
| hsa-miR-28-5p_st | 8.123312706 | 8.360312589 | 6.18153165 | 6.72334116 |
| hsa-miR-28-3p_st | 6.830527448 | 6.407299605 | 3.670113324 | 4.978559366 |
| hsa-miR-29a-star_st | 1.748267835 | 2.022443862 | 0.00546112 | 1.100842105 |
| hsa-miR-29a_st | 13.680311679 | 12.93575986 | 10.37410826 | 11.25896547 |
| hsa-miR-30a_st | 13.05130132 | 12.0149128 | 9.067793345 | 10.88559904 |
| hsa-miR-30a-star_st | 11.09923524 | 10.23710648 | 6.475475296 | 8.452538111 |
| hsa-miR-31_st | 9.619647188 | 8.101965364 | 5.571869825 | 6.623018309 |
| hsa-miR-31-star_st | 2.677595202 | 1.918786537 | 0.864348119 | 0.150686423 |
| hsa-miR-32_st | 1.166563361 | 0.836690028 | −0.373473207 | 0.156618925 |
| hsa-miR-32-star_st | 3.349009922 | 2.998726344 | 1.910695921 | 2.35793901 |
| hsa-miR-33a_st | 0.756242573 | 0.586972588 | −0.061997733 | 0.130096997 |
| hsa-miR-33a-star_st | 2.171593172 | 2.309149985 | 2.365761712 | 0.235256468 |
| hsa-miR-92a-1-star_st | 7.976558893 | 8.085757731 | 8.829550884 | 8.369285127 |
| hsa-miR-92a_st | 11.45554953 | 10.64283391 | 8.777991244 | 9.730037613 |
| hsa-miR-92a-2-star_st | 9.09449318 | 8.980932369 | 9.60951049 | 9.855146145 |
| hsa-miR-93_st | 9.897781626 | 9.310W4419 | 7.657767503 | 7.279514837 |
| hsa-miR-93-star_st | 4.539168591 | 4.339508228 | 4.592281692 | 3.467935794 |
| hsa-miR-95_st | 8.086443894 | 7.894299767 | 5.350165578 | 5.503750948 |
| hsa-miR-96_st | 5.246328885 | 5.913103579 | 2.174771127 | 3.61434929 |
| hsa-miR-96-star_st | 3.683589729 | 3.078257283 | 2.31660147 | 2.846710637 |
| hsa-miR-98_st | 9.554020646 | 9.263015883 | 7.364675578 | 7.611944543 |
| hsa-miR-99a_st | 12.92654872 | 11.84990568 | 8.268397439 | 11.00189645 |
| hsa-miR-99a-star_st | 4.828002098 | 4.245132921 | 2.021521485 | 2.729973125 |
| hsa-miR-100_st | 13.07416587 | 11.57836561 | 8.999592205 | 11.29685279 |
| hsa-miR-100-star_st | 2.776758472 | 1.815709837 | 1.447129752 | 2.093577305 |
| hsa-miR-101-star_st | 2.156574473 | 2.064826172 | 1.749691952 | 1.480993166 |
| hsa-miR-101_st | 4.862240083 | 5.67878915 | 2.290794179 | 3.151929802 |
| hsa-miR-29b-1-star_st | 5.799259779 | 6.040138604 | 4.711839376 | 3.943970648 |
| hsa-miR-29b_st | 11.26582032 | 11.28784293 | 8.132559788 | 8.614078976 |
| hsa-miR-29b-2-star_st | 7.942549396 | 8.190596694 | 5.872576721 | 6.549234697 |
| hsa-miR-103-2-star_st | 3.806061097 | 5.06901149 | 5.718608473 | 4.721570025 |
| hsa-miR-103_st | 12.72342206 | 12.25753739 | 10.23814663 | 10.82241984 |
| hsa-miR-105_st | 1.780457626 | 2.429907613 | 0.757591376 | 1.432887998 |
| hsa-miR-105-star_st | 2.893660905 | 3.016586842 | 2.251257711 | 2.755948349 |
| hsa-miR-106a_st | 10.63596087 | 9.611018622 | 7.50045103 | 7.637153639 |
| hsa-miR-106a-star_st | 1.324502017 | 1.362145996 | 0.572682676 | 1.486150452 |
| hsa-miR-107_st | 12.40095928 | 12.07296552 | 10.12538705 | 10.71427874 |
| hsa-miR-16-2-star_st | 3.21179161 | 2.651708224 | 4.184852424 | 2.497404416 |
| hsa-miR-192_st | 6.947694459 | 7.161749822 | 4.862597963 | 4.341260452 |
| hsa-miR-192-star_st | 3.425682817 | 3.934343476 | 3.148794448 | 3.01583634 |
| hsa-miR-196a_st | 2.358492407 | 2.643032944 | 0.937628053 | 1.307525843 |
| hsa-miR-197_st | 7.888822541 | 7.825775748 | 7.779642934 | 7.19135433 |
| hsa-miR-198_st | 12.04045943 | 11.9966779 | 12.75041315 | 13.27009679 |
| hsa-miR-199a-5p_st | 10.64633486 | 8.622377942 | 4.90208252 | 7.077998366 |
| hsa-miR-199a-3p_st | 12.02658343 | 10.33078881 | 4.677455466 | 7.918235101 |
| hsa-miR-208a_st | 2.353943003 | 2.570362952 | 3.249730736 | 2.240290791 |
| hsa-miR-129-5p_st | 11.14222665 | 11.17707535 | 12.45432092 | 12.5561397 |
| hsa-miR-129-star_st | 4.074661718 | 4.057992762 | 3.085871775 | 3.59562257 |
| hsa-miR-148a-star_st | 4.281543145 | 4.520590327 | 4.271130416 | 3.623976363 |
| hsa-miR-148a_st | 9.953417333 | 10.16665643 | 7.129153945 | 8.872673591 |
| hsa-miR-30c_st | 13.78327519 | 13.25445044 | 11.45097637 | 12.68202159 |
| hsa-miR-30c-2-star_st | 7.552599179 | 7.475386616 | 8.067753581 | 7.42365416 |
| hsa-miR-30d_st | 12.79683497 | 11.72930698 | 9.664865878 | 10.59133543 |
| hsa-miR-30d-star_st | 2.51590425 | 2.159646456 | 0.311206626 | 0.964248287 |
| hsa-miR-139-5p_st | 8.001245997 | 7.12292897 | 3.3781163 | 5.905172009 |
| hsa-miR-139-3p_st | 7.849404245 | 7.344998178 | 8.324273659 | 8.027381198 |
| hsa-miR-147_st | 4.304495246 | 4.188206507 | 3.452829167 | 3.961008631 |
| hsa-miR-7_st | 8.623874667 | 9.966740157 | 4.356877889 | 7.670837028 |
| hsa-miR-7-1-star_st | 5.652010926 | 6.366677192 | 5.310172856 | 5.580097263 |
| hsa-miR-7-2-star_st | 6.720183564 | 7.452894481 | 5.222031053 | 6.186775963 |
| hsa-miR-10a_st | 8.596946346 | 7.735623047 | 5.010738844 | 5.685383228 |
| hsa-miR-10a-star_st | 4.162138961 | 4.068309051 | 4.210632206 | 3.430793953 |
| hsa-miR-10h_st | 9.374464963 | 8.112435575 | 4.892606616 | 6.837048224 |
| hsa-miR-10b-star_st | 3.652693175 | 4.033819926 | 4.540840238 | 2.614435167 |
| hsa-miR-34a_st | 8.857810399 | 9.379133916 | 7.323260879 | 6.853769689 |
| hsa-miR-34a-star_st | 5.680199931 | 5.391458652 | 3.324502218 | 4.12653636 |
| hsa-miR-181a_st | 12.40474448 | 12.27414864 | 8.939235919 | 11.33971306 |
| hsa-miR-181a-2-star_st | 7.475698088 | 8.418060867 | 5.114817398 | 6.56463499 |
| hsa-miR-181b_st | 11.05275668 | 11.00758045 | 8.229311624 | 9.934662767 |
| hsa-miR-181c_st | 7.894910188 | 8.46517983 | 5.921284705 | 7.125742808 |
| hsa-miR-181c-star_st | 5.59934365 | 6.466167443 | 6.307881357 | 6.45427408 |
| hsa-miR-182_st | 7.524211555 | 8.899339742 | 6.702345889 | 7.901414189 |
| hsa-miR-182-star_st | 2.032582862 | 2.131259034 | 2.420276869 | 1.914366526 |
| hsa-miR-183_st | 4.66769888 | 6.196534354 | 5.5375514 | 4.179341993 |
| hsa-miR-183-star_st | 6.541125284 | 6.61979709 | 7.97705585 | 7.539169489 |
| hsa-miR-187-star_st | 9.454544796 | 9.457251443 | 10.26412464 | 10.5096408 |
| hsa-miR-187_st | 3.307934048 | 4.333924292 | 4.44680084 | 2.875484864 |
| hsa-miR-196a-star_st | 3.847132501 | 4.168709422 | 4.243851953 | 4.586396171 |
| hsa-miR-199b-5p_st | 8.067967522 | 4.529025777 | 1.057041185 | 2.807380212 |
| hsa-miR-199b-3p_st | 11.79350667 | 10.21604343 | 5.366542865 | 7.724606411 |
| hsa-miR-203_st | 7.600749502 | 6.444237644 | 5.083414382 | 5.290557638 |
| hsa-miR-204_st | 7.411721036 | 8.37308567 | 3.313088111 | 6.794870821 |
| hsa-miR-205_st | 3.990158386 | 4.644758066 | 3.280489258 | 2.737388309 |
| hsa-miR-205-star_st | 4.930894679 | 4.889438806 | 4.733235709 | 4.600157867 |
| hsa-miR-210_st | 12.60042133 | 12.26927293 | 13.01961584 | 13.19596831 |
| hsa-miR-211_st | 4.298901289 | 5.27868152 | 6.154193409 | 5.562856306 |
| hsa-miR-212_st | 5.923072742 | 6.024019947 | 5.688406394 | 5.171673581 |
| hsa-miR-181a-star_st | 4.510047961 | 5.081552354 | 2.760137254 | 3.616997219 |
| hsa-miR-214-star_st | 5.245165628 | 4.519316903 | 3.855855626 | 3.385412432 |
| hsa-miR-214_st | 10.71268052 | 9.91797503 | 9.298130203 | 9.988812722 |
| hsa-miR-215_st | 3.785440755 | 3.90503273 | 2.553577367 | 2.89366364 |
| hsa-miR-216a_st | 2.340791519 | 1.787989914 | 2.069681995 | 1.72700379 |
| hsa-miR-217_st | 2.620723398 | 2.487393657 | 2.766473151 | 1.612522451 |
| hsa-miR-218_st | 5.153732024 | 3.280111348 | 2.277183206 | 1.176112602 |
| hsa-miR-218-1-star_st | 1.788750208 | 2.035094496 | 2.464154422 | 2.154916125 |
| hsa-miR-218-2-star_st | 3.587807606 | 2.993567475 | 4.378754516 | 2.636383899 |
| hsa-miR-219-5p_st | 0.384446566 | 0.404351952 | 0.367257754 | 0.972382583 |
| hsa-miR-219-1-3p_st | 3.980340937 | 4.428423134 | 5.231668266 | 4.726677154 |
| hsa-miR-220a_st | 1.640339861 | 2.122617922 | 3.214141295 | 2.315753506 |
| hsa-miR-221-star_st | 4.779248214 | 6.058598172 | 4.49356479 | 4.869230705 |
| hsa-miR-221_st | 12.11222807 | 12.00447403 | 11.5409352 | 11.18137776 |
| hsa-miR-222-star_st | 5.613675781 | 5.881210037 | 5.249988899 | 5.792583972 |
| hsa-miR-222_st | 11.94145868 | 11.72274824 | 10.5216812 | 10.95863898 |
| hsa-miR-223-star_st | 2.942046803 | 2.588409082 | 1.805067787 | 2.373048464 |
| hsa-miR-223_st | 9.121444916 | 7.868562683 | 5.322900131 | 8.121239409 |
| hsa-miR-224_st | 6.973063191 | 6.199613757 | 5.165652822 | 5.040369634 |
| hsa-miR-224-star_st | 5.272592145 | 5.165295017 | 5.834069212 | 4.419147963 |
| hsa-miR-200b-star_st | 6.468954011 | 6.923095776 | 5.623171152 | 5.555011611 |
| hsa-miR-200b_st | 11.54311053 | 11.15009086 | 4.73069754 | 8.346543019 |
| hsa-let-7gt_st | 13.90421406 | 13.0451982 | 10.62064191 | 11.92054208 |
| hsa-let-7g-star_st | 5.096948119 | 5.429245988 | 6.361792825 | 5.221381404 |
| hsa-let-7i_st | 14.39693639 | 13.50049584 | 11.15316808 | 11.62843114 |
| hsa-let-7i-star_st | 6.071489133 | 5.736371616 | 5.334407345 | 4.671001479 |
| hsa-miR-1_st | 5.244680127 | 3.281360125 | 1.476561852 | 1.68558141 |
| hsa-miR-15b_st | 11.2379346 | 10.38427128 | 6.998841053 | 9.0013019 |
| hsa-miR-15b-star_st | 3.025151958 | 2.86975036 | 3.343331646 | 2.516608009 |
| hsa-miR-23b-star_st | 5.136783023 | 5.338278615 | 4.77429646 | 4.952859227 |
| hsa-miR-23b_st | 14.88013629 | 13.87806044 | 11.49938663 | 13.22715344 |
| hsa-miR-27b-star_st | 5.860349798 | 5.964703663 | 4.120732943 | 5.199553354 |
| hsa-miR-27b_st | 12.52711974 | 11.7783819 | 7.444099046 | 9.901518021 |
| hsa-miR-30b_st | 12.11162383 | 12.0578777 | 9.35467279 | 10.84041239 |
| hsa-miR-30b-star_st | 8.589807707 | 8.683564575 | 9.55517825 | 9.459831779 |
| hsa-miR-122_st | 3.889363089 | 3.605838016 | 3.4816285 | 3.223910208 |
| hsa-miR-122-star_st | 1.278197653 | 1.643104396 | 1.587150542 | 2.170436413 |
| hsa-miR-124-star_st | 5.158307655 | 4.896442972 | 4.541563143 | 4.747548252 |
| hsa-miR-124_st | 8.125058861 | 7.698790095 | 8.018721262 | 8.380250026 |
| hsa-miR-125b_st | 15.37839298 | 14.14753725 | 11.38400455 | 13.87254178 |
| hsa-miR-125b-1-star_st | 11.77719511 | 11.4391194 | 12 34951892 | 12.58771937 |
| hsa-miR-128_st | 8.293931949 | 8.042393782 | 4.989677167 | 5.505274286 |
| hsa-miR-130a-star_st | 1.93755209 | 1.249764803 | 1.380648632 | 1.992896122 |
| hsa-miR-130a_st | 12.11274077 | 11.1906897 | 6.849160008 | 9.172153736 |
| hsa-miR-132-star_st | 3.028108399 | 2.374363338 | 1.668585891 | 1.551267999 |
| hsa-miR-132_st | 6.752557932 | 6.32167357 | 3.185423803 | 4.498883146 |
| hsa-miR-133a_st | 6.273986193 | 4.966813185 | 3.680650552 | 3.73965703 |
| hsa-miR-135a_st | 1.442072858 | 1.273841152 | 0.453637304 | 0.543004162 |
| hsa-miR-135a-star_st | 12.0538831 | 12.07507252 | 12.82743377 | 13.16900317 |
| hsa-miR-137_st | 0.846258755 | 1.204209133 | 1.178614116 | 1.799875927 |
| hsa-miR-138_st | 12.10998767 | 10.80333116 | 7.833281763 | 9.790973995 |
| hsa-miR-138-2-star_st | 3.725770208 | 3.979475207 | 3.060028878 | 3.201258328 |
| hsa-miR-140-5p_st | 4.445719261 | 4.424472562 | 2.138501593 | 3.070893966 |
| hsa-miR-140-3p_st | 11.03184716 | 10.30751232 | 8.999652593 | 9.601590353 |
| hsa-miR-141-star_st | 2.500630806 | 2.112760706 | 1.951732732 | 1.741624864 |
| hsa-miR-141_st | 9.443828533 | 9.149149511 | 5.261781871 | 5.725930731 |
| hsa-miR-142-5p_st | 2.960603358 | 1.133018333 | 1.910242498 | 1.397685481 |
| hsa-miR-142-3p_st | 0.436913361 | 0.340406984 | 0.836451958 | 0.578413651 |
| hsa-miR-143-star_st | 6.760170454 | 6.589048693 | 5.986821796 | 4.990739243 |
| hsa-miR-143_st | 12.92915097 | 12.04329338 | 8.808331832 | 10.46024021 |
| hsa-miR-144-star_st | 3.333436591 | 2.153373884 | 1.522915426 | 2.466154179 |
| hsa-miR-144_st | 1.061434367 | 1.681963838 | 0.79433018 | 1.194383941 |
| hsa-miR-145_st | 14.45965185 | 12.93912805 | 10.25706229 | 12.22629585 |
| hsa-miR-145-star_st | 5.459142581 | 5.63449693 | 2.591150728 | 3.632172912 |
| hsa-miR-152_st | 10.89837989 | 10.00742135 | 7.650372445 | 8.289858828 |
| hsa-miR-153_st | 1.668336474 | 0.953369544 | 1.48948361 | 1.893133761 |
| hsa-miR-191_st | 13.2433589 | 12.38587658 | 10.07738651 | 11.20928886 |
| hsa-miR-191-star_st | 5.623418766 | 5.752978332 | 7.07585322 | 6.888867875 |
| hsa-miR-9_st | 0.200051971 | −0.001036009 | −0.960945335 | 0.529803707 |
| hsa-miR-9-star_st | 1.963587273 | 1.83701144 | 1.734037582 | 1.383029455 |
| hsa-miR-125a-5p_st | 14.15012293 | 13.29195992 | 11.23217617 | 12.2855968 |
| hsa-miR-125a-3p_st | 11.71601853 | 11.63054033 | 12.64724621 | 12.54399405 |
| hsa-miR-125b-2-star_st | 8.573549739 | 9.333465713 | 9.580022539 | 10.07593647 |
| hsa-miR-126-star_st | 0.827289499 | 0.120824923 | 0.625448597 | 1.179293554 |
| hsa-miR-126_st | 13.8575686 | 13.23283164 | 10.81809315 | 11.65351557 |
| hsa-miR-127-5p_st | 4.998506022 | 6.006863057 | 6.602450467 | 6.112720967 |
| hsa-miR-127-3p_st | 6.299901407 | 5.907298634 | 5.904479109 | 5.479787341 |
| hsa-miR-129-3p_st | 2.892862083 | 3.120739671 | 2.933124943 | 2.303131348 |
| hsa-miR-134_st | 8.13753629 | 8.332181017 | 8.526153922 | 9.022439642 |
| hsa-miR-136_st | 0.448477925 | 0.423496568 | −0.502198971 | −0.338011523 |
| hsa-miR-136-star_st | 1.445986356 | 1.144262388 | 0.561324708 | 0.426275444 |
| hsa-miR-138-1-star_st | 8.172072602 | 7.691401796 | 6.048433919 | 7.06814545 |
| hsa-miR-146a_st | 8.573496171 | 7.898194922 | 3.851725058 | 5.818601668 |
| hsa-miR-146a-star_st | 1.053421606 | 1.03216522 | −1.205914224 | −0.203433661 |
| hsa-miR-149_st | 6.778957741 | 6.680207501 | 6.169160088 | 6.012200051 |
| hsa-miR-149-star_st | 16.63679017 | 15.53649106 | 15.33596685 | 16.2603804 |
| hsa-miR-150_st | 10.53473125 | 8.939972533 | 5.425106806 | 8.681398962 |
| hsa-miR-150-star_st | 13.01287248 | 12.78447723 | 13.49743366 | 13.89141157 |
| hsa-miR-154_st | 3.413323916 | 3.593041837 | 5.961831085 | 4.082194237 |
| hsa-miR-154-star_st | 3.156771561 | 3.151295405 | 3.59428388 | 2.822344894 |
| hsa-miR-184_st | 7.357860868 | 7.399794556 | 8.454248669 | 7.664804763 |
| hsa-miR-185_st | 9.977495773 | 9.288338231 | 8.5661096 | 8.409417083 |
| hsa-miR-185-star_st | 9.542286063 | 9.718285227 | 10.16615653 | 10.43786415 |
| hsa-miR-186_st | 4.957137934 | 4.793091495 | 3.894968795 | 2.474412628 |
| hsa-miR-186-star_st | 0.007183874 | 0.241908136 | −0.098008813 | 0.009371155 |
| hsa-miR-188-5p_st | 6.339496652 | 6.94744166 | 7.035566183 | 7.640720401 |
| hsa-miR-188-3p_st | 2.957769448 | 3.631789929 | 4.133284855 | 4.165261444 |
| hsa-miR-190_st | −0.198930932 | −0.159779808 | −0.348645982 | 0.671634933 |
| hsa-miR-193a-5p_st | 12.78799963 | 12.0592054 | 12.91881181 | 12.88162155 |
| hsa-miR-193a-3p_st | 6.94786343 | 7.043553016 | 4.754336099 | 5.319876239 |
| hsa-miR-194_st | 7.017492693 | 7.375541124 | 5.095710626 | 4.494633514 |
| hsa-miR-195_st | 11.3535786 | 10.67223403 | 8.852363295 | 9.211213692 |
| hsa-miR-195-star_st | 4.186850367 | 5.069942912 | 5.370451755 | 4.387462401 |
| hsa-miR-206_st | 5.151152536 | 5.138838394 | 5.8758.03972 | 6.236512373 |
| hsa-miR-320a_st | 12.75601335 | 12.16351155 | 11.70853241 | 12.0843744 |
| hsa-miR-200c-star_st | 3.247825026 | 3.678655798 | 2.814796301 | 3.413083583 |
| hsa-miR-200c_st | 14.18967553 | 13.3364253 | 11.74345926 | 12.35648154 |
| hsa-miR-155_st | 8.730589163 | 7.866812975 | 7.141686282 | 6.923646844 |
| hsa-miR-155-star_st | 1.708696372 | 2.235610251 | 1.496025962 | 2.65311934 |
| hsa-miR-194-star_st | 8.363420627 | 8.134515441 | 9.19966909 | 9.020668454 |
| hsa-miR-106b_st | 9.443753896 | 8.78678085 | 7.278514787 | 6.290228412 |
| hsa-miR-106b-star_st | 7.65510332 | 7.581459882 | 5.688624992 | 6.877793128 |
| hsa-miR-29c-star_st | 7.730207062 | 7.793705132 | 4.692903533 | 4.43189257 |
| hsa-miR-29c_st | 11.47186231 | 11.14008205 | 7.940987224 | 9.252536909 |
| hsa-miR-30c-1-star_st | 10.03311133 | 10.52696772 | 11.28343633 | 11.29161533 |
| hsa-miR-200a-star_st | 5.386129455 | 5.988508997 | 6.157553693 | 5.631661946 |
| hsa-miR-200a_st | 10.20686655 | 10.01828691 | 3.626107869 | 6.440151698 |
| hsa-miR-302a-star_st | 2.29677852 | 2.417564798 | 1.909985007 | 2.017370516 |
| hsa-miR-302a_st | 1.943212782 | 2.038382097 | 0.847570677 | 1.696802531 |
| hsa-miR-219-2.3p_st | 6.384211662 | 6.84796628 | 7.403509876 | 6.95564895 |
| hsa-miR-34b-star_st | 3.96776278 | 4.450077534 | 1.879668192 | 3.01943276 |
| hsa-miR-34b_st | 6.488782204 | 6.791978638 | 7.622007236 | 5.130741926 |
| hsa-miR-34c-5p_st | 2.265367194 | 2.128246893 | 1.788225075 | 1.187697956 |
| hsa-miR-34c-3p_st | 5.648844974 | 6.034862127 | 6.722810019 | 6.965080001 |
| hsa-miR-299-5p_st | 5.333651397 | 5.183359463 | 5.943575776 | 6.016661536 |
| hsa-miR-299-3p_st | 6.826294477 | 6.947323606 | 7.273500585 | 7.988755583 |
| hsa-miR-301a_st | 2.762532729 | 4.459120962 | 2.121046356 | 1.098728607 |
| hsa-miR-99b_st | 11.29006217 | 11.00373647 | 9.244167232 | 9.673225273 |
| hsa-miR-99b-star_st | 8.123948973 | 7.999178731 | 7.593535396 | 7.586360852 |
| hsa-miR-296-5p_st | 3.5073761 | 3.950104577 | 4.402805231 | 3.556280624 |
| hsa-miR-296-3p_st | 11.84275434 | 11.69343684 | 12.22275471 | 12.4031653 |
| hsa-miR-130b-star_st | 4.3006712 | 4.787864482 | 5.621530214 | 3.505363021 |
| hsa-miR-130b_st | 8.069415741 | 7.966085461 | 6.908520896 | 6.830532025 |
| hsa-miR-30c_st | 9.916286045 | 9.375500175 | 6.920421411 | 7.481851102 |
| hsa-miR-30c-star_st | 9.97393791 | 9.189038026 | 6.439812195 | 7.208651868 |
| hsa-miR-26a-2-star_st | 0.485944001 | 0.742550412 | -0.074385277 | 0.073748819 |
| hsa-miR-361-5p_st | 11.43155668 | 10.65424427 | 8.465368343 | 9.539945275 |
| hsa-miR-361-3p_st | 6.111144691 | 5.503239319 | 4.083824508 | 3.01892708 |
| hsa-miR-362-5p_st | 6.550553799 | 6.802218493 | 5.889063949 | 5.489351263 |
| hsa-miR-362-3p_st | 2.597326861 | 2.782580675 | 1.774631682 | 2.061467188 |
| hsa-miR-363-star_st | 9.805774014 | 10.26169981 | 10.62250696 | 10.34645701 |
| hsa-miR-363_st | 4.612048986 | 4.718494156 | 2.948400454 | 3.863134066 |
| hsa-miR-365_st | 3.251038937 | 2.938390188 | 2.119936982 | 1.189569168 |
| hsa-miR-365-star_st | 11.23199509 | 10.93822755 | 11.95642514 | 12.2120781 |
| hsa-miR-302b-star_st | 3.572040317 | 3.503688803 | 3.600937912 | 3.611414903 |
| hsa-miR.302b_st | 0.033323649 | −0.393170571 | −0.768068235 | −0.343965405 |
| hsa-miR-302c-star_st | 7.760493288 | 7.954012322 | 8.415525113 | 8.391361632 |
| hsa-miR-302c_st | 1.690955922 | 0.933819172 | −0.128455193 | 1.13539498 |
| hsa-miR-302d-star_st | 5.78022579 | 6.445257825 | 8.758538782 | 7.19114014 |
| hsa-miR-302d_st | 0.802038969 | 1.10649924 | −0.791326765 | 0.032393695 |
| hsa-miR-367-star_st | 0.849235527 | 0.8293585 | 1.082638518 | 0.586027922 |
| hsa-miR-367_st | 1.481333238 | 1.677814743 | 1.632439354 | 0.64283786 |
| hsa-miR-376c_st | 4.404289644 | 3.407955004 | 2.884392604 | 2.707283391 |
| hsa-miR-369-5p_st | 3.316313348 | 2.504383425 | 3.419326232 | 2.614176447 |
| hsa-miR-369-3p_st | 0.875573431 | 0.904205693 | 0.342003115 | 1.69160154 |
| hsa-miR-370_st | 10.82969169 | 10.94663096 | 11.16653566 | 12.11914579 |
| hsa-miR-371-5p_st | 11.90934711 | 11.66559637 | 12.56358747 | 12.50287368 |
| hsa-miR-371-3p_st | 4.246194973 | 4.339071588 | 5.306187466 | 5.290201727 |
| hsa-miR-372_st | 1.93509982 | 3.52520786 | 3.019679412 | 3.201351804 |
| hsa-miR-373-star_st | 11.28905989 | 10.93617992 | 11.8747412 | 11.84282021 |
| hsa-miR-373_st | 4.471124146 | 5.482013599 | 4.208403642 | 3.175306358 |
| hsa-miR-3274a_st | 3.360307167 | 2.217833177 | 2.060223584 | 0.56219015 |
| hsa-miR-374a-star_st | 3.067930788 | 2.368403295 | 2.269822931 | 2.335118985 |
| hsa-miR-375_st | 4.525015112 | 4.183518117 | 4.48311008 | 5.255905778 |
| hsa-miR-376a-star_st | 1.018497211 | 0.471021763 | −0.219261959 | −0.180839728 |
| hsa-miR-376a_st | 3.224056473 | 3.299854677 | 3.015643213 | 3.811273006 |
| hsa-miR-377-star_st | 3.994718733 | 4.281197495 | 4.494361719 | 3.463604933 |
| hsa-miR-377_st | 2.325943038 | 2.249148846 | 1.625343402 | 1.177216491 |
| hsa-miR-378-star_st | 5.359169068 | 5.141947206 | 5.961592308 | 5.15803329 |
| hsa-miR-378_st | 8.431838262 | 8.315388395 | 8.236400731 | 8.412290166 |
| hsa-miR-379_st | 4.900723587 | 5.325461826 | 5.632275324 | 5.417886971 |
| hsa-miR-379-star_st | 2.61650132 | 2.13390947 | 2.858338364 | 1.86318168 |
| hsa-miR-380-star_st | 2.161005995 | 1.416477813 | 2.65340506 | 1.85269566 |
| hsa-miR-380_st | 0.47676769 | 0.934629691 | 2.292888159 | 1.328995858 |
| hsa-miR-381_st | 4.631497319 | 5.006094751 | 4.385840756 | 5.188608327 |
| hsa-miR-382_st | 6.707203209 | 6.750814145 | 6.756103695 | 6.746593018 |
| hsa-miR-383_st | 5.302844017 | 5.904030191 | 5.717461328 | 6.112890293 |
| hsa-miR-340_st | 2.683066352 | 3.607835834 | 2.903885794 | 0.794138602 |
| hsa-miR-340-star_st | 1.547599631 | 0.92586989 | 1.272914795 | 1.233087397 |
| hsa-miR-330-5p_st | 5.748135851 | 5.714421368 | 6.233296839 | 5.199805051 |
| hsa-miR-330-3p_st | 6.033132481 | 6.362149319 | 5.607454445 | 5.660787438 |
| hsa-miR-328_st | 7.701886329 | 7.732266124 | 8.251295114 | 8.124428329 |
| hsa-miR-342-5p_st | 4.83711592 | 5.712763178 | 4.603132218 | 3.998680061 |
| hsa-miR-342-3p_st | 10.27603077 | 9.599283817 | 7.855569042 | 8.676174783 |
| hsa-miR-337-5p_st | 2.557578229 | 2.698236984 | 3.148861902 | 2.640529145 |
| hsa-miR-337-3p_st | 3.68332555 | 3.754700167 | 4.266807042 | 4.019484846 |
| hsa-miR-323-5p_st | 10.83663792 | 10.88354069 | 11.8252232 | 11.86697029 |
| hsa-miR-323-3p_st | 3.333838722 | 3.396486231 | 2.850254661 | 3.347886918 |
| hsa-miR-326_st | 7.28135965 | 6.389662408 | 7.215417257 | 6.834417829 |
| hsa-miR-151-5p_st | 11.975172 | 11.06771751 | 8.475622115 | 9.708254107 |
| hsa-miR-151-3p_st | 8.927270185 | 8.232130651 | 5.063012396 | 5.735574621 |
| hsa-miR-135b_st | 1.600616842 | 1.808860403 | 1.46564086 | −0.176298613 |
| hsa-miR-135b-star_st | 6.123792278 | 5.851599245 | 7.937067947 | 7.079881202 |
| hsa-miR-148b-star_st | 1.826120011 | 1.485945874 | 1.189040672 | 2.392090879 |
| hsa-miR-148b_st | 8.968059691 | 8.815196605 | 6.028009553 | 6.695467473 |
| hsa-miR-331-5p_st | 5.753981256 | 6.223558037 | 5.946357974 | 6.27132125 |
| hsa-miR-331-3p_st | 9.234004793 | 9.208916823 | 7.690096766 | 8.36528048 |
| hsa-miR-324-5p_st | 8.594595954 | 8.727897617 | 6.913057884 | 7.122355147 |
| hsa-miR-324-3p_st | 8.61194494 | 8.363209845 | 7.472679198 | 7.759121096 |
| hsa-miR-338-5p_st | 4.491954203 | 4.775885671 | 4.256418432 | 4.653229927 |
| hsa-miR-338-3p_st | 4.065521836 | 4.195282672 | 1.882692004 | 2.113323256 |
| hsa-miR-339-5p_st | 8.205156231 | 8.01607148 | 7.368147594 | 7.003609863 |
| hsa-miR-339-3p_st | 8.637127084 | 8.39558597 | 7.844275571 | 8.290510866 |
| hsa-miR-335_st | 7.641669829 | 8.129625783 | 6.032033565 | 4.932728299 |
| hsa-miR-335-star_st | 21.1661214 | 12.268402667 | 4.94377077 | 1.948471242 |
| hsa-miR-133b_st | 6.784666323 | 5.031078155 | 4.616784403 | 4.724209413 |
| hsa-miR-325_st | 2.853060648 | 3.471916375 | 3.80407475 | 3.874426834 |
| hsa-miR-345_st | 10.59851357 | 10.67912169 | 6.847310947 | 8.454577605 |
| hsa-miR-346_st | 9.555399031 | 8.870529479 | 9.021681576 | 9.960640594 |
| hsa-miR-384_st | 1.123490745 | 0.26558648 | −0.390802462 | 1.069992765 |
| hsa-miR-196b_st | 2.264536781 | 2.484979009 | 2.707146152 | 1.967840156 |
| hsa-miR-196b-star_st | 5.948954768 | 6.276499498 | 6.569665412 | 6.28086413 |
| hsa-miR-422a_st | 8.983806534 | 8.882679037 | 9.87486563 | 9.208426926 |
| hsa-miR-423-5p_st | 13.3735062 | 12.89364059 | 13.50600485 | 13.84236238 |
| hsa-miR-423-3p_st | 10.00437156 | 9.466252103 | 8.634091295 | 8.638450528 |
| hsa-miR-424_st | 5.596922003 | 6.67092464 | 2.194584919 | 2.047772839 |
| hsa-miR-424-star_st | 8.399122813 | 8.384304438 | 7.662456086 | 8.058330009 |
| hsa-miR-425_st | 7.691072671 | 8.199186389 | 5.446019415 | 6.531060359 |
| hsa-miR-425-star_st | 7.838181658 | 7.947874031 | 8.31796297 | 8.198626856 |
| hsa-miR-18b_st | 3.714007434 | 3.553028844 | 2.001169269 | 2.74027829 |
| hsa-miR-18b-star_st | 3.975634589 | 3.837724999 | 5.73034459 | 4.214748359 |
| hsa-miR-20b_st | 8.274423854 | 7.613606136 | 6.318724325 | 5.879053758 |
| hsa-miR-20b-star_st | 4.380467375 | 4.965599058 | 5.053830912 | 5.507004938 |
| hsa-miR-448_st | 1.995628867 | 2.119403855 | 3.636178743 | 1.537743409 |
| hsa-miR-429_st | 4.820608628 | 5.59110814 | 3.147367772 | 1.944800267 |
| hsa-miR-449a_st | 3.222327366 | 3.241181082 | 2.242788242 | 2.976664367 |
| hsa-miR-450a_st | 2.650953631 | 3.024833537 | 3.032072806 | 1.866581529 |
| hsa-miR-431_st | 2.491821857 | 2.382569236 | 2.988633765 | 2.55059584 |
| hsa-miR-431-star_st | 6.237746627 | 6.395154153 | 6.366077539 | 6.752372148 |
| hsa-miR-433_st | 4.136946105 | 4.660273768 | 4.802417626 | 4.354328356 |
| hsa-miR-329_st | 2.746620613 | 2.557765836 | 2.076494177 | 2.898824993 |
| hsa-miR-451_st | 13.68172624 | 12.75740874 | 7.012489589 | 11.30509166 |
| hsa-miR-452_st | 5.693101949 | 5.940767331 | 6.356139299 | 5.429315621 |
| hsa-miR-452-star_st | 4.004729801 | 3.20726597 | 3.286230278 | 3.87379775 |
| hsa-miR-409-5p_st | 3.283946497 | 3.21905172 | 3.518976638 | 3.598641862 |
| hsa-miR-409-3p_st | 5.490975536 | 5.615373048 | 5.252184952 | 5.155386837 |
| hsa-miR-412_st | 3.910187307 | 4.358637998 | 5.938048937 | 4.127117412 |
| hsa-miR-410_st | 3.401374867 | 3.102593357 | 2.564069511 | 3.148024967 |
| hsa-miR-376b_st | 2.037331802 | 1.633547686 | 2.109034746 | 1.384231192 |
| hsa-miR-483-5p_st | 11.48697621 | 11.14013436 | 11.79228324 | 12.10165268 |
| hsa-miR-483-3p_st | 7.015955269 | 6.672237311 | 7.6947126 | 7.117736593 |
| hsa-miR-484_st | 7.179139789 | 7.628582821 | 6.314937919 | 6.491440502 |
| hsa-miR-485-5p_st | 5.865033827 | 6.043636579 | 6.278040564 | 6.186079495 |
| hsa-miR-485-2p_st | 5.040404224 | 5.191017249 | 6.33382879 | 6.544911556 |
| hsa-miR-486-5p_st | 9.945133767 | 8.216680029 | 6.626558893 | 8.443564606 |
| hsa-miR-486-3p_st | 10.30725446 | 9.745482622 | 10.32434641 | 10.91720717 |
| hm-miR-487a_st | 4.950546283 | 5.352456447 | 5.022346627 | 5.099229098 |
| hsa-miR-488-star_st | 2.101418594 | 1.736782612 | 2.192048811 | 2.074310116 |
| hsa-miR-488_st | 2.385195266 | 1.84629391 | 0.522412748 | 0.793672075 |
| hsa-miR-489_st | 7.224259227 | 8.487167896 | 9.392607614 | 9.221319953 |
| hsa-miR-490-5p_st | 6.287414241 | 6.712714336 | 7.789732604 | 7.747411863 |
| hsa-miR-490-3p_st | 3.957729018 | 4.529561833 | 6.269429331 | 5.156304206 |
| hsa-miR-491-5p_st | 9.14801115 | 9.119533094 | 9.360999042 | 9.331565902 |
| hsa-miR-491-3p_st | 4.161097073 | 5.101786149 | 5.183513981 | 4.999811428 |
| hsa-miR-511_st | 0.702313416 | 0.714146383 | −0.243418908 | 0.079447355 |
| hsa-miR-146b-5p_st | 9.015522352 | 9.152902899 | 6.340359326 | 5.681079655 |
| hsa-miR-146b-3p_st | 3.403523326 | 4.319334342 | 3.034035011 | 3.942033319 |
| hsa-miR-202-star_st | −0.408710262 | −1.224903335 | −0.325593377 | −0.997067698 |
| hsa-miR-202_st | 8.173654613 | 8.586726092 | 9.217224764 | 8.8184631738 |
| hsa-miR-492_st | 7.688090156 | 7.704359996 | 9.062426003 | 9.102016572 |
| hsa-miR-493-star_st | 3.162219739 | 3.170230452 | 2.76910378 | 2.59678215 |
| hsa-miR-493_st | 4.465152523 | 5.02433445 | 5.736822861 | 5.478322642 |
| hsa-miR-432_st | 6.572573183 | 6.739568851 | 6.781156057 | 7.532411502 |
| hsa-miR-432-star_st | 4.330503177 | 5.03034875 | 5.436058465 | 5.239478042 |
| hsa-miR-494_st | 14.75246457 | 15.01578591 | 15.17287543 | 16.10271.852 |
| hsa-miR-495_st | 2.677284625 | 2.394071447 | 1.957841643 | 1.519621597 |
| hsa-miR-496_st | 1.491130997 | 1.675203096 | 2.141256899 | 1.53866506 |
| hsa-miR-193b-star_st | 11.57881434 | 11.22118542 | 11.9356515 | 12.12750837 |
| hsa-miR-193b_st | 8.722490265 | 7.933249019 | 6.011209961 | 6.252612011 |
| hsa-miR-497_st | 9.880786896 | 8.888758093 | 6.690177723 | 7.151218198 |
| hsa-miR-497-star_st | 2.728169727 | 2.519892129 | 1.45478758 | 1.511959166 |
| hsa-miR-181d_st | 9.546893657 | 9.474112716 | 7.490449476 | 8.190783076 |
| hsa-miR-512-5p_st | 3.610445142 | 4.250914414 | 5.70436943 | 5.019476144 |
| hsa-miR-512-3p_st | 4.613123718 | 4.549653388 | 4.641703368 | 4.769113756 |
| hsa-miR-498_st | 11.37241328 | 11.06065387 | 11.73356668 | 12.09446051 |
| hsa-miR-520e_st | 1.271983135 | 1.294041938 | −0.268031871 | 1.060488617 |
| hsa-miR-515-5p_st | 2.306234144 | 1.928544491 | 1.402261236 | 1.743039617 |
| hsa-miR-515-3p_st | 2.363556379 | 3.539060893 | 3.713444648 | 2.648821267 |
| hsa-miR-519e-star_st | 4.976779173 | 6.234375268 | 6.044391889 | 6.306589474 |
| hsa-miR-519e_st | 3.968351439 | 3.840298188 | 3.171937727 | 3.235211346 |
| hsa-miR-520f_st | 0.6762583.57 | 1.89807052 | −0.811860466 | 0.887527051 |
| hsa-miR-519c-5p_st | 4.55929431 | 5.018043301 | 6.153401371 | 6.768684196 |
| hsa-miR-519c-3p_st | 0.362559732 | 0.295886916 | −0.028172323 | 0.607261925 |
| hsa-miR-520a-5p_st | 2.858531648 | 3.719555849 | 4.97524749 | 4.02941409 |
| hsa-miR-520a-3p_st | 2.216029214 | 2.164579655 | 1.88957143 | 1.718621096 |
| hsa-miR-526b_st | 4.512010262 | 5.05462741 | 5.432868216 | 6.171072749 |
| hsa-miR-526b-star_st | 1.024741198 | 1.279532309 | 0.222092606 | 0.745519221 |
| hsa-miR-519b-5p_st | 5.688749288 | 5.348697266 | 6.51157094 | 7.042383186 |
| hsa-miR-519b-3p_st | 1.56291148 | 1.77313865 | 0.566468504 | 1.52256108 |
| hsa-miR-525-5p_st | 4.630508828 | 5.476751421 | 6.071568336 | 6.347440019 |
| hsa-miR-525-3p_st | 3.044225068 | 2.49581343 | 3.317340452 | 3.103063132 |
| hsa-miR-523-star_st | 4.75883717 | 4.549634758 | 6.577608902 | 7.095216182 |
| hsa-miR-523_st | 1.257382437 | 1.608987536 | −0.03852437 | 1.966963204 |
| hsa-miR-518f-star_st | 5.222288263 | 6.351974543 | 6.204044626 | 6.484032146 |
| hsa-miR-518f_st | 1.333105212 | 1.069308819 | 0.461487661 | 1.258275453 |
| hsa-miR-520b_st | 1.265926491 | 1.370427544 | 0.11123631 | 1.144638543 |
| hsa-miR-518b_st | 3.626783705 | 3.842392906 | 4.182290718 | 3.907051147 |
| hsa-miR-526a_st | 5.040739311 | 6.01758759 | 6.419561853 | 7.648065313 |
| hsa-miR-520c-5p_st | 4.2371028 | 5.856593262 | 6.884801142 | 7.250926877 |
| hsa-miR-520c-3p_st | 3.379730618 | 3.558563779 | 2.195875869 | 2.89079991 |
| hsa-miR-518c-star_st | 7.788806025 | 8.255909544 | 8.997885104 | 9.251760045 |
| hsa-miR-518c_st | 0.69110614 | 0.098388841 | −0.734749822 | 0.809050159 |
| hsa-miR-524-5p_st | 3.558902371 | 5.01547627 | 5.577985611 | 5.351318587 |
| hsa-miR-524-3p_st | 3.332175704 | 2.969925085 | 3.49591154 | 3.542643801 |
| hsa-miR-517-star_st | 3.881761665 | 4.972207769 | 5.828180436 | 5.099171501 |
| hsa-miR-517a_st | 1.142142988 | 1.77643191 | 1.455968132 | 4.03594404 |
| hsa-miR-519d_st | 3.445149798 | 5.176889255 | 3.77663282 | 4.674648426 |
| hsa-miR-521_st | 3.162040426 | 2.839665167 | 2.431095553 | 2.761564039 |
| hsa-miR-520d-5p_st | 5.099609202 | 5.915113675 | 6.949657545 | 6.505307004 |
| hsa-miR-520d-3p_st | 4.216808958 | 4.934183662 | 5.612221069 | 5.75567015 |
| hsa-miR-517b_st | 2.425572566 | 3.23432469 | 2.962888503 | 4.842949335 |
| hsa-miR-520g_st | 1.434258799 | 2.983828657 | 2.36649476 | 3.356506828 |
| hsa-miR-516b_st | 6.068500901 | 7.048631664 | 7.232776266 | 7.859808933 |
| hsa-miR-516b-star_st | 2.977201837 | 2.828689965 | 2.270424765 | 2.364534038 |
| hsa-miR-518e-star_st | 5.710663209 | 5.621366618 | 6.736633271 | 7.28636198 |
| hsa-miR-518e_st | 1.939216552 | 1.979057411 | 0.582926368 | 2.534633473 |
| hsa-miR-518a-5p_st | 5.484250927 | 6.400779521 | 7.334769917 | 7.987066618 |
| hsa-miR-518a-3p_st | 2.61962248 | 3.114159375 | 3.127147536 | 3.957343392 |
| hsa-miR-518d-5p_st | 4.957013741 | 6.168347881 | 6.993936988 | 7.426922315 |
| hsa-miR-518d-3p_st | 2.569767115 | 2.464411534 | 2.707356601 | 2.869206938 |
| hsa-miR-517e_st | 0.346296634 | 0.469967149 | 0.457766473 | 1.5685395 |
| hsa-miR-520h_st | 2.086135675 | 2.294451586 | 2.886149924 | 2.772852027 |
| hsa-miR-522-star_st | 4.753812187 | 5.334628514 | 5.493407302 | 7.154470214 |
| hsa-miR-522_st | 2.570990253 | 2.257011602 | 2.058867451 | 3.2376085 |
| hsa-miR-519a-star_st | 4.70277895 | 4.871011474 | 6.06749863 | 6.455096822 |
| hsa-miR-519a_st | 1.185992184 | 0.9788824 | −0.092276397 | 1.86904505 |
| hsa-miR-527_st | 6.202731154 | 6.611320914 | 7.978549152 | 8.079381.615 |
| hsa-miR-516a-5p_st | 5.988111823 | 6.58292372 | 7.620995211 | 8.046510697 |
| hsa-miR-516a-3p_st | 2.955268253 | 2.155749326 | 1.752678801 | 2.069355666 |
| hsa-miR-499-5p_st | 2.211139301 | 2.087048811 | 0.201791451 | 1.289218943 |
| hsa-miR-499-3p_st | 2.760633389 | 2.357884381 | 2.209156671 | 1.563268224 |
| hsa-miR-500_st | 6.181665756 | 5.952074625 | 4.773930114 | 4.469984509 |
| hsa-miR-500-star_st | 9.030920841 | 8.538077287 | 8.353384809 | 8.141336827 |
| hsa-miR-501-5p_st | 5.396947659 | 5.675229931 | 4.839233686 | 5.810843576 |
| hsa-miR-501-3p_st | 8.106901495 | 8.273116305 | 8.47027712 | 8.678756618 |
| hsa-miR-502-5p_st | 3.761496028 | 3.867682178 | 2.62117773 | 3.111313633 |
| hsa-miR-502-3p_st | 8.435697346 | 8.477783091 | 7.521004192 | 7.634626573 |
| hsa-miR-503_st | 10.61366553 | 9.865656289 | 11.01753547 | 10.83829654 |
| hsa-miR-504_st | 5.360727245 | 5.236806635 | 5.724072832 | 5.64046857 |
| hsa-miR-505-star_st | 6.932005194 | 7.049431265 | 7.414575416 | 7.008814896 |
| hsa-miR-505_st | 4.55828447 | 5.130852067 | 3.357976966 | 2.248955283 |
| hsa-miR-513a-5p_st | 12.1046364 | 12.66112211 | 13.19600086 | 13.79723245 |
| hsa-miR-513a-3p_st | 1.001508234 | 0.36826761 | 0.200083112 | 0.890134052 |
| hsa-miR-506_st | 3.586773699 | 3.456770276 | 4.080674828 | 3.37322638 |
| hsa-miR-507_st | 0.82044249 | 0.511013834 | 0.803892662 | 1.567600221 |
| hsa-miR-508-5p_st | 7.771968922 | 7.999130437 | 8.619456195 | 9.18480847 |
| hsa-miR-508-3p_st | −0.429103682 | 0.306943741 | −0.322930801 | −0.838581445 |
| hsa-miR-509-5p_st | 3.582480146 | 4.423614723 | 5.188919685 | 5.049664757 |
| hsa-miR-509-3p_st | 4.622828296 | 4.813708522 | 4.679440173 | 4.796024154 |
| hsa-miR-510_st | 7.328737291 | 7.70738716 | 8.680082646 | 8.704086925 |
| hsa-miR-514_st | 1.517871415 | 0.970357311 | 0.008882164 | 1.07072062 |
| hsa-miR-532-5p_st | 8.04219745 | 7.654205403 | 6.731118346 | 6.429236403 |
| hsa-miR-532-3p_st | 8.088159085 | 7.765646662 | 6.932990305 | 7.234312748 |
| hsa-miR-455-5p_st | 3.360490025 | 2.878159212 | 2.341931082 | 1.79726567 |
| hsa-miR-455-3p_st | 8.784896906 | 7.876516996 | 5.796970324 | 6.835533264 |
| hsa-miR-539_st | 3.319843346 | 3.021805043 | 3.240219606 | 2.662709981 |
| hsa-miR-544_st | 0.610179798 | 0.055713025 | 0.353780267 | 0.470410288 |
| hsa-miR-545-star_st | 0.878761813 | 1.366812241 | 1.433002819 | 1.398577412 |
| hsa-miR-545_st | 1.417327023 | 0.848667315 | −0.469400152 | 0.685363067 |
| hsa-miR-487b_st | 6.229736949 | 7.131982957 | 7.012964999 | 6.107746135 |
| hsa-miR-551a_st | 3.810754317 | 3.444889898 | 4.467543844 | 3.855289781 |
| hsa-miR-552_st | 3.548337305 | 4.219585302 | 6.620574711 | 4.315280695 |
| hsa-miR-553_st | 2.717037555 | 2.50042178 | 1.236581881 | 2.015763555 |
| hsa-miR-554_st | 4.079876404 | 4.351764039 | 5.486304696 | 4.54855859 |
| hsa-miR-92b-star_st | 15.25687132 | 14.51975526 | 14.24544199 | 15.43594874 |
| hsa-miR-92b_st | 9.448707813 | 9.024157953 | 8.521543981 | 8.417767394 |
| hsa-miR-555_st | 3.303768932 | 3.39716382 | 3.547694409 | 3.860847964 |
| hsa-miR-556-5p_st | 1.7058265/5 | 0.901111201 | −0.08062856 | 0.060370126 |
| hsa-miR-556-3p_st | 0.888064703 | 1.007056819 | 1.287607208 | 1.122278349 |
| hsa-miR-557_st | 12.15429565 | 11.7510164 | 12.44979903 | 12.59507443 |
| hsa-miR-558_st | 0.99008644 | 1.02821929 | 1.666709879 | 0.443088246 |
| hsa-miR-559_st | 1.573919842 | 0.977742479 | 1.270535961 | 2.036913442 |
| hsa-miR-561_st | 1.327771814 | 0.354692286 | −0.252898983 | 0.374073519 |
| hsa-miR-562_st | 1.518323396 | 1.1865286 | 0.92061375 | 12.31612962 |
| hsa-miR-563_st | 2.366869458 | 2.057381088 | 2.564706102 | 1.942708993 |
| hsa-miR-564_st | 11.70682995 | 11.92451583 | 12.18794141 | 12.62466832 |
| hsa-miR-566_st | 7.556852398 | 6.901466031 | 6.725504249 | 7.034973361 |
| hsa-miR-567_st | 3.222862586 | 3.210992352 | 2.410976448 | 2.929558822 |
| hsa-miR-568_st | 0.821082826 | 0.697107064 | 0.601235793 | 1.808933599 |
| hsa-miR-551b-star_st | 10.491309 | 10.58412643 | 10.89387543 | 10.89588663 |
| hsa-miR-551b_st | 2.100430676 | 2.891180147 | 0.718002497 | 1.671583047 |
| hsa-miR-569_st | 2.498686023 | 2.243654785 | 1.372552073 | 2.561962106 |
| hsa-miR-570_st | 1.781283916 | 1.696051832 | 1.535931251 | 1.349462665 |
| hsa-miR-571_st | 3.963162148 | 4.132617125 | 3.019950301 | 3.034975685 |
| hsa-miR-572_st | 14.43182634 | 13.71088503 | 14.2457319 | 14.88966559 |
| hsa-miR-573_st | 1.169562209 | 1.017935318 | 0.852214036 | 0.346158858 |
| hsa-miR-574-5p_st | 7.811959857 | 7.98916159 | 7.878216552 | 7.772829319 |
| hsa-miR-574-3p_st | 10.16348091 | 9.453413187 | 7.237664895 | 9.105149162 |
| hsa-miR-575_st | 12.53841855 | 12.51675986 | 12.92647795 | 13.53376601 |
| hsa-miR-576-5p_st | 2.959168927 | 2.988904507 | 3.890220935 | 2.82754008 |
| hsa-miR-576-3p_st | 4.61167322 | 4.443105867 | 3.999404885 | 3.63794014 |
| hsa-miR-577_st | 0.397892873 | 0.608677929 | 0.717954196 | 0.639014922 |
| hsa-miR-578_st | 2.986800913 | 3.012545428 | 2.119884786 | 2.449467327 |
| hsa-miR-579_st | 1.996007301 | 1.68293746 | 0.789697177 | 2.369853703 |
| hsa-miR-580_st | 1.518864571 | 1.171931991 | 1.192003735 | 1.31445676 |
| hsa-miR-581_st | −0.009483798 | 0.220997198 | 0.103549424 | 0.124103514 |
| hsa-miR-582-5p_st | 0.866582877 | 0.830708181 | 1.087744659 | 0.019599761 |
| hsa-miR-582-3p_st | 2.804606468 | 2.644514108 | 2.154790439 | 2.289700181 |
| hsa-miR-583_st | 4.4781731989 | 5.479572088 | 6.882908902 | 6.87822049 |
| hsa-miR-584_st | 6.644179071 | 7.104145107 | 7.548949577 | 7.402872696 |
| hsa-miR-585_st | 3.158587512 | 3.165437498 | 3.147818657 | 2.985731738 |
| hsa-miR-548a-3p_st | 1.596364318 | 1.280390342 | 0.132761099 | −0.335731056 |
| hsa-miR-586_st | 2.344988904 | 1.728708548 | 0.48216527 | 1.825982972 |
| hsa-miR-587_st | 2.086996148 | 3.103481595 | 2.984174143 | 3.125139244 |
| hsa-miR-548b-5p_st | 3.465767147 | 2.722024623 | 2.403642792 | 1.566159523 |
| hsa-miR-548b-3p_st | 2.043194018 | 2.543230261 | 1.033817391 | 1.495422227 |
| hsa-miR-588_st | 4.943891068 | 4.805973606 | 4.345704292 | 5.270198861 |
| hsa-miR-589_st | 2.739390334 | 3.948850954 | 3.903403608 | 2.3832335 |
| hsa-miR-589-star_st | 4.500657837 | 4.823910282 | −5.707038982 | 5.314492845 |
| hsa-miR-550_st | 10.66826656 | 11.3973578 | 12.21595233 | 12.28480266 |
| hsa-miR-550-star_st | 3.714581867 | 4.884868474 | 5.142945638 | 4.334713003 |
| hsa-miR-590-5p_st | 1.98745085 | 1.936296236 | 1.781864558 | 0.77192895 |
| hsa-miR-590-3p_st | 0.220784646 | 1.087278605 | 0.698480139 | 0.807890324 |
| hsa-miR-591_st | 2.506993096 | 2.230807199 | 2.955099903 | 1.916071265 |
| hsa-miR-592_st | 0.856312933 | 0.503674622 | 0.681908324 | 0.914049708 |
| hsa-miR-593-star_st | 7.523822906 | 8.565775959 | 9.330630646 | 9.46260744 |
| hsa-miR-593_st | 5.659663898 | 5.951821439 | 5.07649263 | 4.919194593 |
| hsa-miR-595_st | 7.082613683 | 7.148557786 | 7.353500516 | 7.052813346 |
| hsa-miR-596_st | 8.109116874 | 7.910067374 | 8.332356726 | 8.524722219 |
| hsa-miR-597_st | 1.031983187 | 1.033526899 | 1.520749238 | 1.083128096 |
| hsa-miR-598_st | 5.527440081 | 6.129854325 | 4.138866681 | 4.490006912 |
| hsa-miR-599_st | 1.752546588 | 1.08583459 | 0.137668964 | 0.560388914 |
| hsa-miR-548a-5p_st | 4.651150278 | 4.10266644 | 2.776504015 | 3.432013932 |
| hsa-miR-600_st | 4.371316783 | 4.788674785 | 6.516436231 | 5.384855557 |
| hsa-miR-601_st | 5.330621343 | 5.738662546 | 6.199343069 | 6.219808196 |
| hsa-miR-602_st | 13.85687299 | 13.20154422 | 13.99376486 | 14.5311121 |
| hsa-miR-603_st | 1.200987355 | 1.569354372 | 0.624070025 | 0.764590817 |
| hsa-miR-604_st | 8.250573194 | 8.734633676 | 8.716558006 | 8.912973539 |
| hsa-miR-605_st | 4.762110098 | 5.451833299 | 7.021082306 | 5.597642095 |
| hsa-miR-606_st | 1.956299706 | 1.465643263 | 1.459278287 | 1.620299621 |
| hsa-miR-607_st | 2.775308709 | 2.011249011 | 1.580127315 | 1.535594714 |
| hsa-miR-608_st | 9.526159938 | 9.39345738 | 10.05676087 | 10.07620903 |
| hsa-miR-609_st | 2.676169118 | 2.081134797 | 2.070836211 | 3.228669955 |
| hsa-miR-610_st | 4.442368862 | 4.302014088 | 5.46768961 | 4.786566398 |
| hsa-miR-611_st | 7.676732242 | 7.214564104 | 8.16685875 | 8.565322 |
| hsa-miR-612_st | 11.28493978 | 11.40116443 | 12.37190388 | 12.59802188 |
| hsa-miR-613_st | 3.626478649 | 4.302167609 | 3.554477082 | 3.398286727 |
| hsa-miR-614_st | 4.899056394 | 5.14676756 | 6.314433096 | 6.566224485 |
| hsa-miR-615-5p_st | 10.70528033 | 10.49117366 | 11.25971341 | 11.50707402 |
| hsa-miR-615-3p_st | 7.061438593 | 6.919840213 | 6.809181541 | 7.582428254 |
| hsa-miR-616-star_st | 3.925541918 | 4.23459399 | 3.785917853 | 3.145642082 |
| hsa-miR-616_st | 7.393772862 | 7.591586629 | 8.231298975 | 7.415244839 |
| hsa-miR-548c-5p_st | 8.5613925861 | 7.832850048 | 4.691005635 | 3.829045136 |
| hsa-miR-548c-3p_st | 0.736443408 | 0.546496528 | −0.588414441 | −0.436561068 |
| hsa-miR-617_st | 4.803263515 | 5.813230395 | 7.545030169 | 5.023409312 |
| hsa-miR-618_st | 2.798379037 | 2.536342891 | 3.042670049 | 1.748360898 |
| hsa-miR-619_st | 3.701312525 | 3.815113951 | 3.806825073 | 3.107222527 |
| hsa-miR-620_st | 2.242724989 | 2.110160229 | 3.442336134 | 2.930335488 |
| hsa-miR-621_st | 3.321457023 | 3.137499425 | 4.800835983 | 2.618824186 |
| hsa-miR-622_st | 7.984455635 | 8.776017804 | 9.076905618 | 9.158094544 |
| hsa-miR-623_st | 9.122791966 | 8.982954985 | 9.336689173 | 9.604868556 |
| hsa-miR-624-star_st | 4.008527721 | 3.335362819 | 2.977009647 | 3.438746624 |
| hsa-miR-624_st | 3.033924569 | 2.753069575 | 2.551250032 | 2.824142589 |
| hsa-miR-625_st | 6.96091631 | 6.89362271 | 7.358124032 | 6.597953836 |
| hsa-miR-625-star_st | 3.363817431 | 3.23553708 | 3.971467473 | 3.279300369 |
| hsa-miR-626_st | 1.818829098 | 1.774383519 | 1.121493213 | 1.746503401 |
| hsa-miR-627_at | 1.643651941 | 1.429134668 | 0.01527961 | −0.226278232 |
| hsa-miR-628-5p_st | 2.767676762 | 1.830261519 | 1.213993981 | 1.671852923 |
| hsa-miR-628-3p_st | 8.84914342 | 9.325776414 | 11.0997708. | 9.765209158 |
| hsa-miR-629_st | 7.461767261 | 7.563889839 | 6.988666557 | 7.343668188 |
| hsa-miR-629-star_st | 4.847277296 | 5.259245462 | 5.556362691 | 5.028311772 |
| hsa-miR-630_st | 5.626814971 | 6.479810631 | 7.82364249 | 7.039566349 |
| hsa-miR-631_st | 5.748364171 | 6.125445177 | 6.901221164 | 6.662426692 |
| hsa-miR-33b_st | 1.509508535 | 0.597724381 | 0.1115375055 | 0.483613654 |
| hsa-miR-33b-star_st | 6.213020706 | 6.597048611 | 6.633265095 | 7.139089388 |
| hsa-miR-632_st | 3.283328448 | 3.986108139 | 4.302072377 | 3.124612602 |
| hsa-miR-633_st | 1.678200227 | 1.243106048 | 1.755430508 | 2.247671028 |
| hsa-miR-634_st | 5.16327607 | 5.539080519 | 6.163966823 | 5.862631655 |
| hsa-miR-635_st | 4.499166082 | 4.904284167 | 5.075898545 | 5.087467107 |
| hsa-miR-636_st | 9.649983122 | 8.957490053 | 9.710756198 | 9.955664693 |
| hsa-miR-637_st | 12.70648413 | 12.2219657 | 13.1203992 | 13.34664817 |
| hsa-miR-638_st | 16.82583215 | 15.68760048 | 15.33503592 | 16.35842034 |
| hsa-miR-639_st | 8.686046313 | 8.806347295 | 9.225462631 | 9.674096305 |
| hsa-miR-640_st | 6.406036892 | 6.517050896 | 7.235019894 | 6.770630498 |
| hsa-miR-641_st | 3.061321722 | 3.210692345 | 3.105240992 | 3.024443534 |
| hsa-miR-642_st | 2.833885529 | 3.110872839 | 2.57091629 | 2.966592075 |
| hsa-miR-643_st | 0.864443095 | 2.1863099 | 2.687478297 | 2.303251861 |
| hsa-miR-644_st | 2.090300639 | 2.48190247 | 0.262453437 | 0.8952705 |
| hsa-miR-645_st | 2.530746232 | 2.683701611 | 2.689088189 | 1.513029116 |
| hsa-miR-646_st | 3.397822452 | 2.941166876 | 2.384898604 | 3.321528582 |
| hsa-miR-647_st | 4.003560464 | 6.188915448 | 5.448412248 | 4.754702836 |
| hsa-miR-648_st | 7.335062637 | 7.712786623 | 8.528042054 | 8.021695799 |
| hsa-miR-649_st | 3.67971451 | 4.198223404 | 3.090713694 | 3.668543956 |
| hsa-miR-650_st | 10.1481418 | 9.474639965 | 10.25182582 | 10.57492613 |
| hsa-miR-651_st | 0.445277076 | 1.053738486 | 1.05086477 | 0.605013171 |
| hsa-miR-652_st | 7.949560485 | 7.853109136 | 6.582726191 | 6.645879685 |
| hsa-miR-548d-5p_st | 7.360267394 | 6.658029445 | 2.990819369 | 4.001901958 |
| hsa-miR-548d-3p_st | 1.994773664 | 2.16081734 | 0.983842672 | 0.875984184 |
| hsa-miR-661_st | 7.44540529 | 7.253284372 | 7.11020542 | 7.369940684 |
| hsa-miR-662_st | 6.034252134 | 6.093382182 | 6.122712094 | 6.513324242 |
| hsa-miR-663_st | 16.5934618 | 15.42423353 | 15.33625634 | 16.24447672 |
| hsa-miR-449b_st | 4.797141722 | 4.79909231 | 4.58595706 | 4.672664052 |
| hsa-miR-449b-star_st | 7.857283601 | 7.848662638 | 8.216760343 | 8.506307971 |
| hsa-miR-653_st | 1.106907123 | 0.256644068 | 2.325152193 | 0.438370826 |
| hsa-miR-411_st | 3.335691735 | 2.967234747 | 2.759995351 | 2.973115051 |
| hsa-miR-411-star_st | 3.715134428 | 3.821225916 | 3.870658719 | 3.850245308 |
| hsa-miR-654-5p_st | 10.15895553 | 10.6489257 | 11.5110892 | 12.33782824 |
| hsa-miR-654-3p_st | 3.661695204 | 5.619365951 | 4.816144494 | 3.000416362 |
| hsa-miR-655_st | 0.906080776 | 0.718999725 | 0.067276738 | 0.146702674 |
| hsa-miR-656_st | 3.099123522 | 2.173228154 | 3.269099496 | 1.543781571 |
| hsa-miR-549_st | 2.467652829 | 2.21387869 | 2.787928792 | 2.33283136 |
| hsa-miR-657_st | 3.724109369 | 3.682879335 | 5.076183796 | 3.923230815 |
| hsa-miR-658_st | 12.19377733 | 11.77696576 | 12.28022453 | 12.8377025 |
| hsa-miR-659_st | 10.44708232 | 10.43430391 | 11.0251027 | 11.26107126 |
| hsa-miR-660_st | 7.114293509 | 6.625166558 | 4.914623022 | 5.01971782 |
| hsa-miR-421_st | 6.107800551 | 6.267098582 | 5.654146122 | 4.939031495 |
| hsa-miR-542-5p_st | 7.377661175 | 7.523626465 | 7.206159892 | 7.754228592 |
| hsa-miR-542-3p_st | 4.69734634 | 5.66580513 | 5.96380632 | 5.56620297 |
| hsa-miR-758_st | 2.450277501 | 2.570506438 | 3.1715716 | 1.325962266 |
| hsa-miR-1264_st | 2.332774695 | 2.098542415 | 1.93800895 | 2.120349242 |
| hsa-miR-671-5p_st | 11.4532377 | 11.06728759 | 11.55459236 | 12.20431395 |
| hsa-miR-671-3p_st | 5.868324251 | 6.769824161 | 7.404163047 | 7.06378556 |
| hsa-miR-668_st | 8.102091255 | 8.427992362 | 9.077610623 | 9.134023394 |
| hsa-miR-767-5p_st | 2.579006794 | 2.754504547 | 2.003248059 | 2.947028227 |
| hsa-miR-767-3p_st | 1.965860018 | 2.350472892 | 3.027868983 | 1.966075806 |
| hsa-miR-1224-5p_st | 13.05045054 | 12.74162752 | 13.6112501 | 13.73720261 |
| hsa-miR-1224-3p_st | 5.249762161 | 5.358271898 | 7.090449559 | 5.46927206 |
| hsa-miR-320b_st | 12.06436497 | 11.45217194 | 10.63174016 | 10.92937027 |
| hsa-miR-320c_st | 11.66249355 | 10.71003057 | 9.78356249 | 10.15481048 |
| hsa-miR-1296_st | 6.828462317 | 6.197309016 | 7.420111805 | 6.421199534 |
| hsa-miR-1468_st | 4.919981554 | 5.463877754 | 6.877835487 | 6.221438866 |
| hsa-miR-1323_st | 7.304856542 | 7.772463463 | 7.999766605 | 8.488849167 |
| hsa-miR-1271_st | 6.05405897 | 6.619878747 | 6.794560279 | 6.270065049 |
| hsa-miR-1301_st | 7.197210379 | 7.90420429 | 7.946110036 | 7.880225706 |
| hsa-miR-454-star_st | 2.144625725 | 1.712211583 | 1.499354102 | 1.61798444. |
| hsa-miR-454_st | 3.964494136 | 4.960265049 | 2.591447474 | 1.065389007 |
| hsa-miR-1185_st | 1.151554229 | 1.032298365 | 0.412654372 | 0.224122481 |
| hsa-miR-449c_st | 5.959341131 | 6.395147941 | 7.772084379 | 7.396201984 |
| hsa-miR-449c-star_st | 3.71277301 | 3.96095334 | 4.557226471 | 4.575132331 |
| hsa-miR-1283_st | 1.915133717 | 2.303974627 | 3.389586864 | 2.566177153 |
| hsa-miR-769-5p_st | 6.371491588 | 7.167932566 | 6.760065421 | 6.391929608 |
| hsa-miR-769-3p_st | 9.547719104 | 9.439108531 | 10.32388326 | 10.44138785 |
| hsa-miR-766_st | 7.339629768 | 7.523930737 | 7.971647646 | 7.700352376 |
| hsa-miR-762_st | 16.76868616 | 15.6041217 | 15.33720724 | 16.34358027 |
| hsa-miR-802_st | 1.080486387 | 0.391286922 | 0.512161962 | 0.679653059 |
| hsa-miR-670_st | 4.222012993 | 4.701603256 | 4.130291807 | 4.64576156 |
| hsa-miR-1298_st | 3.229934102 | 4.000206453 | 4.872480826 | 3.259557889 |
| hsa-miR-2113_st | 4.801006816 | 5.521521473 | 5.088639255 | 5.440513025 |
| hsa-miR-761_st | 7.020354487 | 7.276838509 | 7.91447577 | 8.272703255 |
| hsa-miR-764_st | 5.119055355 | 5.978268059 | 6.567472495 | 5.979927114 |
| hsa-miR-759_st | 1.198727868 | 1.198186082 | 0.907009283 | 1.449717547 |
| hsa-miR-765_st | 8.725017911 | 9.145093167 | 9.697710346 | 9.625867139 |
| hsa-miR-770-5p_st | 6.235127247 | 6.771835188 | 7.276804581 | 7.000849746 |
| hsa-miR-675_st | 13.62609191 | 12.76832827 | 13.67923956 | 13.99321113 |
| hsa-miR-675-star_st | 7.272125734 | 6.925201712 | 7.899073782 | 8.337199696 |
| hsa-miR-298_st | 9.852365218 | 10.18389802 | 10.64209271 | 11.03798492 |
| hsa-miR-891a_st | 5.817426468 | 5.770974993 | 7.229102928 | 6.084532474 |
| hsa-miR-300_st | 4.764690634 | 4.767748344 | 5.451135946 | 5.901921162 |
| hsa-miR-886-5p_st | 12.70950005 | 11.83542348 | 12.06637747 | 12.51293883 |
| hsa-miR-886-3p_st | 11.14869336 | 10.30518738 | 9.210951617 | 9.915750803 |
| hsa-miR-892a_st | 2.636830189 | 4.017622897 | 5.472504169 | 4.560156529 |
| hsa-miR-220c_st | 3.379918141 | 3.50429268 | 3.973960106 | 3.414552275 |
| hsa-miR-450b-5p_st | 0.328948416 | 0.046942734 | 0.162292995 | 0.625735886 |
| hsa-miR-450b-3p_st | 0.984425175 | 0.737770107 | 0.051873416 | 0.591649314 |
| hsa-miR-874_st | 11.90706167 | 11.76221816 | 12.35629337 | 12.91327086 |
| hsa-miR-890_st | 4.389964436 | 5.523298561 | 7.073328483 | 5.176748711 |
| hsa-miR-891b_st | 2.746131046 | 2.662836578 | 1.815137697 | 2.622007111 |
| hsa-miR-220c_st | 4.817604743 | 6.263178844 | 6.512521322 | 5.396386738 |
| hsa-miR-888_st | 1.781695783 | 1.585274458 | 1.658933193 | 0.88584169 |
| hsa-miR-888-star_st | 0.916043305 | 1.069958076 | 0.914226168 | 0.838247206 |
| hsa-miR-892b_st | 3.469869279 | 3.904086945 | 4.29455937 | 2.838950513 |
| hsa-miR-541-star_st | 6.359838087 | 6.221440777 | 6.242503547 | 6.510524164 |
| hsa-miR-541_st | 7.707957871 | 8.087011184 | 8.480149195 | 8.693805869 |
| hsa-miR-889_st | 1.578287762 | 1.046492892 | 2.086323564 | 1.937226347 |
| hsa-miR-875-5p_st | 0.315608033 | 0.706653686 | 4.139245494 | 0.535795599 |
| hsa-miR-875-3p_st | 2.999220755 | 2.729779235 | 2.155760224 | 3.435552947 |
| hsa-miR-876-5p_st | 0.03863183 | -0.304959898 | 0.403996083 | 0.480722016 |
| hsa-miR-876-3p_st | 1.373732646 | 1.442643055 | 0.983839486 | 1.071856781 |
| hsa-miR-708_st | 7.239197915 | 6.835198868 | 6.483775913 | 4.722705328 |
| hsa-miR-708-star_st | 3.849764031 | 3.866066809 | 3.580852098 | 2.892062786 |
| hsa-miR-147b_st | 3.914269803 | 3.870121614 | 4.771212594 | 4.056059076 |
| hsa-miR-190b_st | 0.633446086 | 1.076370957 | 0.775052509 | −0.249151231 |
| hsa-miR-744_st | 14.70217707 | 14.20123681 | 14.96715719 | 15.58817853 |
| hsa-miR-744-star_st | 3.713698066 | 3.758788685 | 4.554413642 | 4.350294361 |
| hsa-miR-885-5p_st | 7.172481189 | 7.341928357 | 8.649609276 | 7.821084222 |
| hsa-miR-885-3p_st | 14.09623533 | 13.2666511 | 13.95380378 | 14.39408241 |
| hsa-miR-877_st | 11.93586796 | 11.70879748 | 12.33726818 | 12.50737987 |
| hsa-miR-877-star_st | 7.277016312 | 6.390417395 | 7.83670507 | 7.201516652 |
| hsa-miR-887_st | 8.8331618694 | 8.449282294 | 9.33228137 | 9.396635569 |
| hsa-miR-665_st | 12.45853312 | 12.06068253 | 12.41395325 | 12.68316187 |
| hsa-miR-873_st | 4.272598796 | 4.193212241 | 4.251622638 | 4.019190514 |
| hsa-miR-543_st | 3.708955709 | 3.3480405 | 3.83803881 | 4.102454384 |
| hsa-miR-374b_st | 6.048632241 | 5.787739384 | 3.872193699 | 2.758113275 |
| hsa-miR-374b-star_st | 1.545771098 | 1.327410017 | 0.562223604 | 1.262982618 |
| hsa-miR-760_st | 10.67016869 | 10.30453529 | 10.74309848 | 11.29343081 |
| hsa-miR-301b_st | 0.004172902 | 1.134541019 | 1.236881731 | -0.108888319 |
| hsa-miR-216b_st | 4.017565261 | 3.428607839 | 3.649467597 | 2.877467589 |
| hsa-miR-208b_st | 2.664578067 | 2.8389751 | 0.910518462 | 1.753974803 |
| hsa-miR-920_st | 10.26048059 | 10.02648387 | 10.80713868 | 11.06642267 |
| hsa-miR-921_st | 10.25497246 | 10.74968211 | 11.75043863 | 11.89166336 |
| hsa-miR-922_st | 4.509270408 | 5.233178939 | 6.277185644 | 5.983522058 |
| hsa-miR-924_st | 2.019863486 | 2.19707223 | 3.002759242 | 0.748347347 |
| hsa-miR-509-3-5p_st | 5.606708197 | 6.223341462 | 7.805786002 | 5.637988843 |
| hsa-miR-933_st | 10.96044315 | 10.55859856 | 10.64715278 | 10.79824735 |
| hsa-miR-934_st | 3.354013522 | 3.46976687 | 3.189667527 | 3.81129798 |
| hsa-miR-935_st | 6.678257946 | 6.45936146 | 7.548521133 | 7.344681574 |
| hsa-miR-936_st | 10.12298367 | 10.17478509 | 10.7359425 | 10.91971242 |
| hsa-miR-937_st | 4.240432727 | 4.893285672 | 5.720364603 | 4.553469406 |
| hsa-miR-938_st | 3.91172587 | 4.345856717 | 5.334771867 | 4.442201953 |
| hsa-miR-939_st | 13.53649246 | 13.04497356 | 13.50054334 | 14.15250009 |
| hsa-miR-940_st | 8.26629469 | 7.917507393 | 9.077161476 | 8.892749052 |
| hsa-miR-941_st | 5.14039694 | 5.401732722 | 5.317688737 | 5.557745712 |
| hsa-miR-942_st | 1.746705837 | 1.697845435 | 1.638036919 | 0.782667492 |
| hsa-miR-943_st | 8.133539192 | 7.806899416 | 8.663679447 | 8.567512531 |
| hsa-miR-944_st | 1.760020567 | 1.4865282 | −0.426772601 | 1.545835088 |
| hsa-miR-297_st | 4.090194732 | 5.95594855 | 4.212958805 | 3.933114008 |
| hsa-miR-1178_st | 3.583097511 | 3.829677464 | 4.728832479 | 4.162261373 |
| hsa-miR-1179_st | 1.499578099 | 1.668009207 | −0.240283308 | 0.71332853 |
| hsa-miR-1180_st | 9.7896753781 | 9.451569101 | 9.367611923 | 10.16423389 |
| hsa-miR-1181_st | 10.01447497 | 9.601251221 | 10.02537281 | 10.09593038 |
| hsa-miR-1182_st | 10.7606795 | 10.50515791 | 11.33452074 | 11.24237013 |
| hsa-miR-1183_st | 10.3242268 | 10.67532292 | 11.04819082 | 11.04137448 |
| hsa-miR-1184_st | 3.711344923 | 4.269689798 | 4.231342595 | 4.182820617 |
| hsa-miR-1225-5p_st | 14.11053686 | 13.70917522 | 14.24746333 | 14.88474749 |
| hsa-miR-1225-32p_st | 7.991840582 | 7.785029773 | 8.582877853 | 8.680460756 |
| hsa-miR-1226-star_st | 11.02877444 | 10.72178519 | 11.45018231 | 11.79218749 |
| hsa-miR-1226_st | 5.700582042 | 5.744766534 | 7.0405091 | 6.242981796 |
| hsa-miR-1227_st | 5.419469141 | 5.198321665 | 6.021184152 | 5.665351209 |
| hsa-miR-1228-star_st | 16.70998293 | 15.55700397 | 15.33610805 | 16.29285962 |
| hsa-miR-1228_st | 8.154115643 | 8.05923496 | 8.284488649 | 8.878458863 |
| hsa-miR-1229_st | 8.03656271 | 7.605598641 | 8.315984806 | 7.609823243 |
| hsa-miR-1231_st | 14.21257807 | 13.63274086 | 14.38096772 | 14.78253075 |
| hsa-miR-1233_st | 7.17634291 | 6.918075954 | 7.521408561 | 7.823261022 |
| hsa-miR-1234_st | 6.838286834 | 6.80815887 | 7.585403874 | 7.017561509 |
| hsa-miR-1236_st | 5.841932948 | 5.717808985 | 6.363944409 | 5.551508776 |
| hsa-miR-1237_st | 7.99816422 | 8.096619309 | 8.139073365 | 8.61766465 |
| hsa-miR-1238_st | 6.806674748 | 7.019020562 | 7.852386277 | 7.015796807 |
| hsa-miR-1200_st | 4.60755795 | 5.354495868 | 5.087865523 | 5.090030192 |
| hsa-miR-1201_st | 7.928667307 | 7.927733969 | 7.372510068 | 6.824235646 |
| hsa-miR-1202_st | 13.06650356 | 12.92261707 | 13.72162959 | 14.04562654 |
| hsa-miR-1203_st | 8.216345523 | 8.469704043 | 8.921408438 | 8.929213375 |
| hsa-miR-663b_st | 10.08383 | 9.73883975 | 10.8925643 | 10.97017249 |
| hsa-miR-1204_st | 4.89894805 | 5.088187073 | 4.726236643 | 5.184858194 |
| hsa-miR-1205_st | 3.342607697 | 3.657960242 | 3.133278231 | 3.21001883 |
| hsa-miR-1206_st | 0.912534017 | 0.715262617 | −0.781103034 | 0.337732972 |
| hsa-miR-1207-5p_st | 15.46101297 | 14.73993958 | 14.96362568 | 15.62123855 |
| hsa-miR-1207-3p_st | 3.904235798 | 3.872213017 | 4.314777158 | 3.971824924 |
| hsa-miR-1208_st | 8.35520478 | 8.416007134 | 9.709637838 | 9.475237418 |
| hsa-miR-548e_st | 0.46241271 | 0.39142689 | 1.017450221 | 0.461710119 |
| hsa-miR-548j_st | 3.361188914 | 3.292840372 | 3.921747375 | 2.451554908 |
| hsa-miR-1285_st | 13.01632775 | 12.8823899 | 12.43062011 | 12.99252508 |
| hsa-miR-1286_st | 7.72852305 | 8.445567843 | 9.230146776 | 8.834770442 |
| hsa-miR-1287_st | 5.471271745 | 5.960019992 | 6.681396732 | 5.853883448 |
| hsa-miR-1289_st | 4.56369306 | 4.963962371 | 3.695735726 | 3.225426247 |
| hsa-miR-1290_st | 9.679564994 | 9.129036839 | 9.398244283 | 9.307488772 |
| hsa-miR-1291_st | 8.168479109 | 8.358091441 | 9.22049167 | 8.296236635 |
| hsa-miR-548k_st | 2.197201364 | 2.325718735 | 1.490168219 | 2.353650507 |
| hsa-miR-1293_st | 7.631046340 | 7.669139533 | 8.11335445 | 8.466871205 |
| hsa-miR-1294_st | 5.436385596 | 5.959692915 | 5.561478995 | 5.133624265 |
| hsa-miR-1295_st | 5.297172118 | 5.500607795 | 6.896104851 | 6.733670936 |
| hsa-miR-1297_st | 0.724144921 | 1.169591166 | 0.330944799 | -0.496106028 |
| hsa-miR-1299_st | 5.204375499 | 5.767106251 | 4.258952329 | 4.366795007 |
| hsa-miR-5481_st | 4.273381644 | 4.296765959 | 2.785056688 | 3.807303151 |
| hsa-miR-1302_st | 1.75649963 | 1.286548082 | 3.71112815 | 2.06904518 |
| hsa-miR-1303_st | 10.50497028 | 10.33983581 | 10.56147319 | 10.66925816 |
| hsa-miR-1304_st | 5.648867949 | 6.701391191 | 7.186285314 | 7.102162117 |
| hsa-miR-1305_st | 2.310964882 | 2.35679225 | 2.059400448 | 1.67784167 |
| hsa-miR-1243_st | 3.368599396 | 2.909771223 | 2.925218666 | 3.156172105 |
| hsa-miR-548f_st | 1.254985225 | 0.845929419 | 1.05633651 | 0.350027612 |
| hsa-miR-1244_st | 7.632693704 | 8.451290155 | 7.268389986 | 8.873274812 |
| hsa-miR-1245_st | 3.648606204 | 2.927243577 | 2.937517715 | 3.360656507 |
| hsa-miR-1246_st | 13.78938377 | 13.04291866 | 13.15280909 | 12.6537567 |
| hsa-miR-1247_st | 6.28046736 | 6.379706246 | 7.333077533 | 7.043184828 |
| hsa-miR-1248_st | 9.028266837 | 9.932013427 | 9.955835036 | 10.22919251 |
| hsa-miR-1249_st | 4.742773346 | 4.470797979 | 5.066666152 | 4.681310445 |
| hsa-miR-1250_st | 8.406365311 | 8.481852979 | 8.966489878 | 9.178783164 |
| hsa-miR-1251_st | 4.662359354 | 4.851673773 | 3.421764485 | 3.194277395 |
| hsa-miR-1253_st | 4.067602802 | 4.454960059 | 5.178877179 | 4.549201013 |
| hsa-miR-1254_st | 10.69293115 | 10.934926113 | 11.41367662 | 12.14160589 |
| hsa-miR-1255a_st | 3.917838318 | 5.4239959 | 4.425751658 | 4.609682763 |
| hsa-miR-1256_st | 2.980699524 | 3.107340676 | 2.729576419 | 2.11550905 |
| hsa-miR-1257_st | 3.33890951 | 4.006488792 | 4.102969069 | 4.177521699 |
| hsa-miR-1258_st | 3.697035298 | 4.244841938 | 5.035523494 | 3.605472347 |
| hsa-miR-1259_st | 2.219917081 | 1.698737213 | 1.411795384 | 1.236252404 |
| hsa-miR-1260_st | 10.75426184 | 10.61282162 | 9.540151665 | 10.20221475 |
| hsa-miR-548g_st | 0.961406784 | 0.154572422 | 0.016211565 | −0.304197401 |
| hsa-miR-1261_st | 7.008745335 | 7.735547252 | 8.833477227 | 7.932451799 |
| hsa-miR-1262_st | 2.516339214 | 2.610070218 | 3.045336382 | 2.513164148 |
| hsa-miR-1263_st | 3.413790905 | 3.307066474 | 3.785548065 | 2.579193679 |
| hsa-miR-548n_st | 1.488868639 | 1.494784413 | 0.259442315 | 1.987819487 |
| hsa-miR-548m_st | 2.058863636 | 1.687188637 | 2.351456914 | 2.122010686 |
| hsa-miR-1265_st | 4.719051244 | 4.852673648 | 5.417890945 | 4.781806321 |
| hsa-miR-548o_st | 1.819011213 | 1.641218643 | 0.996206023 | 1.171508815 |
| hsa-miR-1266_st | 8.749567976 | 8.586819418 | 9.23915796 | 9.289911436 |
| hsa-miR-1267_st | 3.881619018 | 3.317034777 | 4.098373022 | 4.099285048 |
| hsa-miR-1268_st | 15.9790372 | 15.11718597 | 15.20533578 | 15.97885655 |
| hsa-miR-1269_st | 4.776676915 | 5.020923709 | 5.529737932 | 5.288180901 |
| hsa-miR-1270_st | 6.199106888 | 6.303482794 | 6.586428453 | 6.563158996 |
| hsa-miR-1272_st | 3.270869025 | 3.122875308 | 4.620269657 | 3.156542523 |
| hsa-miR-1273_st | 11.02246563 | 10.76989738 | 10.39353704 | 10.95862079 |
| hsa-miR-1274a_st | 9.568747066 | 10.03465896 | 9.383120549 | 9.743879148 |
| hsa-miR-548b_st | 3.275704381 | 2.932913652 | 1.13343668 | 0.013115665 |
| hsa-miR-1275_st | 14.07330883 | 13.81655531 | 14.24148814 | 14.68065084 |
| hsa-miR-1276_st | 5.635431364 | 5.797376185 | 7.219162417 | 6.368853534 |
| hsa-miR-302e_st | 2.395226428 | 2.88511818 | 2.436093274 | 3.665971316 |
| hsa-miR-302f_st | 1.020223788 | 1.013751006 | 1.244476073 | 1.068236078 |
| hsa-miR-1277_st | 2.764665269 | 1.438185557 | 1.566373136 | 1.4.4382788 |
| hsa-miR-548p_st | 1.180958451 | 1.305949463 | 0.674521162 | 1.517150123 |
| hsa-miR-548i_st | 4.578266987 | 4.396822381 | 2.639131646 | 3.220080058 |
| hsa-miR-1278_st | 3.925672707 | 3.174846046 | 1.817023541 | 2.837937964 |
| hsa-miR-1279_st | 3.190088856 | 3.024442755 | 1.422201255 | 0.523641598 |
| hsa-miR-12746_st | 13.25598939 | 13.50959829 | 12.97000789 | 13.29480599 |
| hsa-miR-1281_st | 9.059000565 | 9.095417735 | 9.384833818 | 9.599101646 |
| hsa-miR-1282_st | 3.192302676 | 4.288601151 | 1.982558325 | 3.706753823 |
| hsa-miR-1284_st | 3.024413459 | 4.37722344 | 4.515529492 | 4.31737603 |
| hsa-miR-1288_st | 3.731935286 | 3.76649828 | 5.728396309 | 4.650410363 |
| hsa-miR-1292_st | 12.43088819 | 12.34116862 | 13.1636083 | 13.5621493 |
| hsa-miR-1252_st | 1.653467614 | 0.880759235 | 1.882882368 | 1.457760139 |
| hsa-miR-1255b_st | 7.300654252 | 8.295850717 | 7.321072569 | 7.02013921 |
| hsa-miR-1280_st | 15.46704804 | 14.82832318 | 14.03923877 | 14.75703853 |
| hsa-miR-1308_st | 14.98486222 | 15.01417758 | 14.62676488 | 14.97388107 |
| hsa-miR-664-star_st | 9.993835053 | 9.712708523 | 10.18021905 | 9.777048041 |
| hsa-miR-664_st | 5.051341942 | 5.751036681 | 5.761666935 | 5.779450298 |
| hsa-miR-1306_st | 6.840617021 | 7.053952682 | 7.103387956 | 7.475237452 |
| hsa-miR-1307_st | 14.2941406 | 13.37617881 | 14.13392948 | 1.432507148 |
| hsa-miR-513b_st | 9.926543823 | 10.28739809 | 11.57297791 | 11.98510846 |
| hsa-miR-513c_st | 8.063941849 | 9.023749356 | 9.626994734 | 9.898204496 |
| hsa-miR-1321_st | 7.099585987 | 7.598151531 | 8.808100161 | 8.330093258 |
| hsa-miR-1322_st | 3.626969454 | 3.525184531 | 3.724091248 | 3.872489407 |
| hsa-miR-720_st | 15.41912763 | 14.87662038 | 14.37444695 | 14.84592235 |
| hsa-miR-1197_st | 2.133959742 | 1.688860402 | 1.605150748 | 2.309062372 |
| hsa-miR-1324_st | 1.769124533 | 1.695610846 | 1.099782949 | 1.517074083 |
| hsa-miR-1469_st | 16.72095847 | 15.57718604 | 15.33635317 | 16.32007345 |
| hsa-miR-1470_st | 5.603682335 | 5.74074909 | 5.828480765 | 6.337599843 |
| hsa-miR-1471_st | 1.039409495 | 10.68249991 | 11.94599153 | 12.0706761 |
| hsa-miR-1537_st | 1.365680003 | 0.861392991 | 0.942752856 | 1.188012555 |
| hsa-miR-1538_st | 7.529837108 | 7.837174942 | 8.453519663 | 8.564029327 |
| hsa-miR-1539_st | 5.844954457 | 5.671325275 | 7.004200358 | 6.598818739 |
| hsa-miR-103-as_st | 2.127161342 | 2.889842322 | 2.760745957 | 2.231528645 |
| hsa-miR-320d_st | 10.049061 | 9.763778363 | 7.677381821 | 8.409056802 |
| hsa-miR-1825_st | 6.856302384 | 6.699015439 | 7.037075726 | 7.243565051 |
| hsa-miR-1826_st | 16.32585487 | 15.70328241 | 15.33522687 | 16.3575971 |
| hsa-miR-1827_st | 43.741478428 | 4.954485077 | 3.479671646 | 3.82309643 |
| hsa-miR-1908_st | 16.81281513 | 15.64217652 | 15.3303654 | 16.35595952 |
| hsa-miR-1909-star_st | 9.119022776 | 8.92317744 | 9.330025332 | 9.411065623 |
| hsa-miR-1909_st | 15.12419687 | 14.40011948 | 14.93985824 | 15.42788542 |
| hsa-miR-1910_st | 10.20234338 | 9.926037288 | 10.61378857 | 10.64724514 |
| hsa-miR-1911_st | 3.145527282 | 3.184645854 | 5.130815829 | 4.107002671 |
| hsa-miR-1911-star_st | 4.658075907 | 5.030702773 | 4.064546793 | 5.255819332 |
| hsa-miR-1912_st | 4.001384093 | 3.865746486 | 3.538355087 | 4.118885677 |
| hsa-miR-1913_st | 8.237616705 | 7.952887331 | 8.80795441 | 8.603837596 |
| hsa-miR-1914_st | 6.903125285 | 7.359554324 | 6.656591439 | 6.081670273 |
| hsa-miR-1914-star_st | 12.662557 | 12.55414759 | 13.07866111 | 13.78611978 |
| hsa-miR-1915-star_st | 7.720172427 | 7.788545722 | 8.742783194 | 8.178105895 |
| hsa-miR-1915_st | 16.81079247 | 15.63888351 | 15.33533993 | 16.35558 |
| hsa-miR-1972_st | 13.29544678 | 13.42957619 | 12.16183818 | 12.94796176 |
| hsa-miR-1973_st | 14.01930413 | 14.46947987 | 15.32646231 | 15.74214039 |
| hsa-miR-1975_st | 16.32404312 | 15 36795277 | 14.79178069 | 15 99366727 |
| hsa-miR-1976_st | 7.959859616 | 7.835742095 | 8.074508458 | 8.208211194 |
| hsa-miR-1979_st | 15.58724906 | 14.98172603 | 14.26634492 | 15.08869296 |
| hsa-miR-2052_st | 0.132426186 | 0.439437169 | 0.267544675 | 0.608661$84 |
| hsa-miR-2053_st | 1.635318468 | 1.630887974 | 0.868080463 | 2.828516262 |
| hsa-miR-2054_st | 0.12102467 | −0.794225032 | −1.407328249 | −0.145001587 |
| hsa-miR-2110_st | 10.64853729 | 10.56131206 | 11.36224787 | 11.73149438 |
| hm-miR-2114_st | 2.417797389 | 2.147652852 | 1.274853735 | 2.507157259 |
| hsa-miR-2114-star_st | 4.979326639 | 6.024583986 | 7.102254105 | 6.954035675 |
| hsa-miR-2115_st | 1.975770368 | 1.701632793 | 2.369701056 | 0.825623318 |
| hsa-miR-2115-star_st | 7.272849194 | 7.728794325 | 9.633485123 | 8.915777083 |
| hsa-miR-2116_st | 4.294566102 | 5.746923153 | 6.490850977 | 5.986766953 |
| hsa-miR-2116-star_st | 3.627304794 | 4.451414431 | 5.14923881 | 4.91427796 |
| hsa-miR-2117_st | 1.970867356 | 2.150486173 | 2.174498923 | 1.828557754 |
| hsa-miR-548q_st | 7.781288274 | 8.039882909 | 7.948350835 | 8.391793027 |
| hsa-miR-2276_st | 9.677141282 | 10.07997224 | 10.96562624 | 10.79733286 |
| hsa-miR-2277_st | 8.926268827 | 8.666541945 | 8.946248836 | 9.643003749 |
| hsa-miR-2278_st | 5.44540097 | 5.449001332 | 6.22166905 | 5.4222975 |
| hsa-miR-711_st | 11.33694248 | 11.33689883 | 12.33858614 | 12.54151351 |
| hsa-miR-718_st | 7.33468596 | 7.043672525 | 8.490981779 | 7.878328285 |
| hsa-miR-2861_st | 16.81757584 | 15.64957484 | 15.33661258 | 16.35623765 |
| hsa-miR-2909_st | 3.194000343 | 2.733268516 | 2.76760842 | 2.494159926 |
| hsa-miR-3115_st | 2.08057033 | 1.771668275 | 1.07413404 | 1.341353926 |
| hsa-miR-3116_st | 5.643945211 | 5.87107786 | 6.322297739 | 6.272952137 |
| hsa-miR-3117_st | 2.631995899 | 2.965426903 | 2.438042157 | 3.286417281 |
| hsa-miR-3118_st | 2.092779489 | 1.902207128 | 3.037413928 | 1.639121764 |
| hsa-miR-3119_st | 0.738891056 | 1.337859578 | 0.459193204 | 1.222256601 |
| hsa-miR-3120_st | 5.382424543 | 6.105824215 | 6.885072781 | 6.261522297 |
| hsa-miR-3121_st | 5.865046105 | 7.246201672 | 7.681663936 | 7.657149204 |
| hsa-miR-3122_st | 11.0863169 | 11.05485269 | 11.10914063 | 12.55042481 |
| hsa-miR-3123_st | 0.735367678 | 0.835267971 | 1.745365347 | 0.673378873 |
| hsa-miR-3124_st | 11.68283721 | 1.136797365 | 12.52820842 | 12.48815411 |
| hsa-miR-548s_st | 1.442633509 | 1.472518154 | 1.172116719 | 1.26473859 |
| hsa-miR-3125_st | 6.504162127 | 7.182266558 | 7.520218037 | 7.417503971 |
| hsa-miR-3126-5p_st | 10.96810365 | 11.62186652 | 12.68781638 | 13.00605153 |
| hsa-miR-3126-3p_st | 3.953319103 | 3.978451587 | 5.048331457 | 5.129238795 |
| hsa-miR-3127_st | 8.884563625 | 9.039912725 | 9.64981247 | 9.9412665534 |
| hsa-miR-3128_st | 2.854019924 | 2.623850359 | 3.476188214 | 2.377058234 |
| hsa-miR-3129_st | 3.377202117 | 3.953676414 | 3.337570019 | 3.298642869 |
| hsa-miR-3130-5p_st | 6.104011466 | 6.703341513 | 7.899651325 | 7.250658705 |
| hsa-miR-3130-3p_st | 7.781764224 | 7.520785865 | 8.373633709 | 8.032454092 |
| hsa-miR-3131_st | 11.95047502 | 11.54631808 | 12.47524853 | 12.675821367 |
| hsa-miR-3132_st | 8.461240608 | 8.736381457 | 9.440632407 | 9.926082683 |
| hsa-miR-3133_st | 2.378042587 | 2.295880487 | 2.882253985 | 2.433449311 |
| hsa-miR-378b_st | 8.699729699 | 8.528200972 | 9.474499779 | 9.107606241 |
| hsa-miR-3134_st | 1.620075696 | 1.279377813 | 0.444280401 | 1.693810819 |
| hsa-miR-3135_st | 6.2135514745 | 6.446723785 | 6.288928729 | 6.294295833 |
| hsa-miR-466_st | 5.583333986 | 5.641216216 | 5.925404787 | 4.497290916 |
| hsa-miR-3136_st | 5.709042043 | 5.853991317 | 6.790102542 | 7.003281584 |
| hsa-miR-544b_st | 2.556910659 | 2.429889652 | 2.973163073 | 2.011956337 |
| hsa-miR-3137_st | 10.58265506 | 10.80228301 | 11.04354704 | 11.66753526 |
| hsa-miR-3138_st | 10.20602609 | 10.11848337 | 11.40768658 | 11.89898801 |
| hsa-miR-3139_st | 1.896074286 | 2.852669535 | 3.669149066 | 1.918817763 |
| hsa-miR-3140_st | 4.222628987 | 4.471333241 | 4.561171795 | 5.121162607 |
| hsa-miR-548t_st | 2.525714261 | 2.317318822 | 1.852011068 | 1.331908769 |
| hsa-miR-3141_st | 15.71898682 | 14.80622412 | 15.09937963 | 15.74324263 |
| hsa-miR-3142_st | 1.205131996 | 0.701501475 | 1.508902008 | 2.015208406 |
| hsa-miR-3143_st | 0.555087091 | 0.589265729 | −0.314516863 | 0.1.80454299 |
| hsa-miR-548u_st | 0.997890624 | 1.161559813 | 1.575939429 | 0.851874369 |
| hsa-miR-3144-5p_st | 3.926118612 | 4.520530436 | 5.82417076 | 4.984584952 |
| hsa-miR-3144-3p_st | 2.282236025 | 2.790490565 | 2.372629955 | 3.021075861 |
| hsa-miR-3145_st | −0.111673215 | 0.353988674 | 0.763924371 | −0.286856322 |
| hsa-miR-1273c_st | 9.882253246 | 9.964363674 | 10.69050718 | 10.54306794 |
| hsa-miR-3146_st | 2.945613978 | 3.625310743 | 3.479988314 | 3.574430026 |
| hsa-miR-3147_st | 11.31223075 | 11.06988344 | 11.26902777 | 11.74209153 |
| hsa-miR-548v_st | 2.920060947 | 2.993303609 | 2.05241956 | 2.208413496 |
| hsa-miR-3148_st | 4.167148141 | 5.000873101 | 5.431043849 | 4.917964986 |
| hsa-miR-3149_st | 2.754983043 | 2.867249042 | 2.011214842 | 2.10613195 |
| hsa-miR-3150_st | 9.615649728 | 9.11211859 | 9.746588367 | 9.932034858 |
| hsa-miR-3151_st | 11.44924489 | 11.11472788 | 11.80353343 | 11.82346138 |
| hsa-miR-3152_st | 6.541702375 | 4.690797349 | 2.984411612 | 7.814784315 |
| hsa-miR-3153_st | 9.354932089 | 9.202213372 | 9.87868729 | 10.01096999 |
| hsa-miR-3074_st | 4.501612427 | 4.491314484 | 4.753265571 | 3.719386519 |
| hsa-miR-3154_st | 11.49774802 | 11.12714529 | 11.72343524 | 11.99313811 |
| hsa-miR-3155_st | 6.565851986 | 6.889214589 | 7.542009555 | 7.662245725 |
| hsa-miR-3156_st | 9.928795173 | 9.903434578 | 10.05090179 | 9.892850521 |
| hsa-miR-3157_st | 5.305026019 | 5.074194853 | 6.611393865 | 5.805131658 |
| hsa-miR-3158_st | 3.955107788 | 4.291612812 | 5.901754412 | 4.579903461 |
| hsa-miR-3159_st | 4.035939071 | 4.32783237 | 5.414366599 | 3.342490764 |
| hsa-miR-3160_st | 5.280229075 | 5.375606752 | 4.249289009 | 4.859477079 |
| hsa-miR-3161_st | 6.017152498 | 6.591561574 | 7.038460631 | 7.443594734 |
| hsa-miR-3162_st | 13.78868534 | 13.3045883 | 13.82420336 | 14.13551663 |
| hsa-miR-3163_st | 7.335080458 | 7.788269015 | 8.516681454 | 8.219031745 |
| hsa-miR-3164_st | 5.482057365 | 6.455302725 | 6.73749629 | 7.063559483 |
| hsa-miR-3165_st | 6.348356354 | 6.919507228 | 8.384407521 | 8.460337368 |
| hsa-miR-3166_st | 4.255315724 | 3.566827129 | 4.193637407 | 4.062160516 |
| hsa-miR-1260b_st | 14.13645693 | 13.31450831 | 13.27181796 | 14.15228502 |
| hsa-miR-3167_st | 1.359122744 | 1.259165833 | 2.177125463 | 1.568751667 |
| hsa-milt-3168_st | 2.122248955 | 2.176182938 | 2.567775628 | 2.471731792 |
| hsa-miR-3169_st | 3.90841288 | 3.555686667 | 3.133065499 | 3.575791301 |
| hsa-miR-3170_st | 6.004392899 | 5.864261805 | 6.880459689 | 6.613095322 |
| hsa-miR-3171_st | 2.667171702 | 2.387096828 | 1.198033798 | 2.248682366 |
| hsa-miR-3172_st | 35.92077025 | 14.68880989 | 15.11734161 | 15.14609441 |
| hsa-miR-3173_st | 9.048177133 | 8.968976691 | 9.411201517 | 9.520804629 |
| hsa-miR-1193_st | 9.911480551 | 9.636563813 | 10.09103872 | 10.42165416 |
| hsa-miR-323b-5p_st | 6.945202112 | 7.524213663 | 8.223721373 | 7.548105125 |
| hsa-miR-323b-3p_st | 2.419752805 | 2.294720423 | 2.165882863 | 2.014582413 |
| hsa-miR-3174_st | 5.377129063 | 6.508082051 | 7.645911237 | 6.573067633 |
| hsa-miR-3175_st | 12.93931604 | 12.66498524 | 13.32768879 | 13.59467072 |
| hsa-miR-3176_st | 10.72480996 | 10.37095989 | 10.89275954 | 10.66606667 |
| hsa-miR-3177_st | 11.36567443 | 11.28019025 | 12.03209576 | 12.3665468 |
| hsa-miR-3178_st | 16.21059466 | 15.19604729 | 15.3340853 | 16.14169734 |
| hsa-miR-3179_st | 2.887270068 | 3.337387994 | 3.816917295 | 4.193563805 |
| hsa-miR-3180-5p_st | 7.635121632 | 7.430306941 | 8.44798072 | 8.310428335 |
| hsa-miR-3180-3p_st | 15.1994894 | 14.37446565 | 14.90086765 | 15.42058031 |
| hsa-miR-548w_st | 6.673203927 | 6.550087426 | 4.029213156 | 4.404701612 |
| hsa-miR-3181_st | 8.467396794 | 8.054873157 | 9.078799228 | 8.846671007 |
| hsa-miR-3182_st | 3.16117371 | 3.220767719 | 2.395839408 | 2.521380618 |
| hsa-miR-3183_st | 3.297133256 | 3.742008798 | 4.994021886 | 5.047533899 |
| hsa-miR-3194_st | 9.463583975 | 9.242817485 | 10.1691606 | 10.22021817 |
| hsa-miR-3185_st | 13.6640327 | 14.75812583 | 15.16272631 | 15.86427235 |
| hsa-miR-3065-5p_st | 3.451786816 | 3.147668435 | 1.69895205 | 2.551370871 |
| hsa-miR-3065-3p_st | 3.019294463 | 4.097916906 | 4.821272566 | 4.186438595 |
| hsa-miR-3186-5p_st | 3.620689213 | 4.401528046 | 4.470002531 | 3.498190896 |
| hsa-miR-3186-3p_st | 8.496605877 | 8.552527963 | 9.640403704 | 8.863884674 |
| hsa-miR-3187_st | 12.59381048 | 12.34988788 | 13.34906934 | 13.52923318 |
| hsa-miR-3188_st | 12.96506681 | 12.6688561 | 13.5748588 | 13.8780603 |
| hsa-miR-3189_st | 7.249371513 | 7.344889529 | 8.312516751 | 7.957371797 |
| hsa-miR-320c_st | 10.48111268 | 9.327977586 | 8.212206741 | 8.319047394 |
| hsa-miR-3190-5p_st | 9.652359814 | 9.807611909 | 10.21870217 | 10.61183419 |
| hsa-miR-3190-3p_st | 9.892239451 | 9.9510742 | 19.64439145 | 10.77693315 |
| hsa-miR-3191_st | 11.0830004 | 11.19477116 | 11.86977093 | 12.48403807 |
| hsa-miR-3192_st | 8.470003126 | 8.637509546 | 9.490772075 | 8.981398358 |
| hsa-miR-3193_st | 5.769719923 | 6.007491391 | 6.666289427 | 6.283734856 |
| hsa-miR-3194_st | 10.01116325 | 10.33787585 | 11.08111668 | 11.64941776 |
| hsa-miR-3193_st | 12.65371718 | 12.62349079 | 13.04519708 | 13.80441822 |
| hsa-miR-3196_st | 16.8253773 | 15.68293987 | 15.33594888 | 16.35720843 |
| hsa-miR-548x_st | 0.1248527 | 0.312276603 | 1.236305876 | 0.548993064 |
| hsa-miR-3197_st | 15.25351605 | 14.76280554 | 15.11766207 | 15.88380859 |
| hsa-miR-3198_st | 9.516123713 | 9.56709137 | 10.37375244 | 10.46456015 |
| hsa-miR-3199_st | 4.306521728 | 4.45176047 | 6.043481966 | 5.592119239 |
| hsa-miR-3200_st | 3.070729684 | 3.441688654 | 2.531463452 | 2.96866272 |
| hsa-miR-3201_st | 3.76016476 | 3.987363786 | 4.096188264 | 4.153246398 |
| hsa-miR-514b-5p_st | 9.663950632 | 9.962281065 | 10.49206243 | 10.36380426 |
| hsa-miR-415b-3p_st | 1.073350543 | 1.487872809 | 1.130047673 | 1.655528889 |
| hsa-miR-3202_st | 7.812265519 | 7.542395552 | 8.417045091 | −8.354353044 |
| hsa-miR-1273d_st | 11.77028387 | 11.6709639 | 10.59217381 | 11.00159289 |
| hsa-miR-4295_st | 2.113017483 | 1.650716633 | 2.719843815 | 1.455272015 |
| hsa-miR-4296_st | 7.098732239 | 7.175574426 | 7.64324757 | 7.614420881 |
| hsa-miR-4297_st | 4.595170908 | 5.554352442 | 6.149366799 | 6.142865613 |
| hsa-miR-378c_st | 8.53033554 | 8.217178808 | 8.710138926 | 8.827477528 |
| hsa-miR-4293_st | 3.990289864 | 4.089600536 | 5.156839509 | 4.729844589 |
| hsa-miR-4294_st | 7.382348631 | 7.481199535 | 8.499596592 | 8.55304756 |
| hsa-miR-4301_st | 2.344239551 | 2.017064066 | 1.655613837 | 2.131812365 |
| hsa-miR-4299_st | 12.11777373 | 12.73119768 | 13.32859055 | 13.27151577 |
| hsa-miR-4298_st | 13.08995053 | 12.77697854 | 13.31206505 | 13.59236967 |
| hsa-miR-4300_st | 7.268698715 | 7.456844024 | 8.803179915 | 8.552925365 |
| hsa-miR-4304_st | 5.787527746 | 5.870801767 | 6.139488076 | 6.495595167 |
| hsa-miR-4302_st | 4.945013657 | 5.014106472 | 6.310027957 | 5.637091239 |
| hsa-miR-4303_st | 5.143181804 | 5.971380004 | 6.908483815 | 6.373314087 |
| hsa-miR-4305_st | 3.286512482 | 3.690368588 | 5.070309387 | 3.030223299 |
| hsa-miR-4306_st | 9.216708673 | 9.639101996 | 9.50259055 | 9.658865651 |
| hsa-miR-4309_st | 3.726743084 | 3.074433833 | 2.914805719 | 3.865614409 |
| hsa-miR-4307_st | 1.828997925 | 3.050393996 | 1.540194065 | 1.61005368 |
| hsa-miR-4308_st | 1.557421473 | 1.66087641 | 1.665246583 | −0.098516564 |
| hsa-miR-4310_st | 2.808520703 | 2.819603543 | 4.635336896 | 2.949926376 |
| hsa-miR-4311_st | 4.650444661 | 5.128340853 | 5.377693933 | 4.349796503 |
| hsa-miR-4312_st | 6.127439891 | 6.607877135 | 7.883909102 | 6.69264288 |
| hsa-miR-4313_st | 5.562308902 | 5.707320771 | 6.723536981 | 5.423431952 |
| hsa-miR-4315_st | 2.243168815 | 2.653119732 | 2.987270516 | 2.620692902 |
| hsa-miR-4316_st | 7.483488844 | 7.26321408 | 8.080742863 | 8.393380861 |
| hsa-miR-4314_st | 8.40250987 | 8.17214178 | 9.604787925 | 9.303692084 |
| hsa-miR-4318_st | 2.654578226 | 3.623085724 | 3.084754315 | 2.280820913 |
| hsa-miR-4319_st | 3.102730444 | 3.371998623 | 3.791579557 | 3.694410614 |
| hsa-miR-4320_st | 2.880254648 | 2.82420049 | 3.402652417 | 2.017410231 |
| hsa-miR-4317_st | 8.33557065 | 8.837623024 | 10.0554715 | 9.656000175 |
| hsa-miR-4322_st | 12.715955512 | 12.20471674 | 13.17334529 | 13.34673293 |
| hsa-miR-4321_st | 9.955181327 | 9.908439559 | 10.65127282 | 10.84096092 |
| hsa-miR-4323_st | 5.147613483 | 5.822933319 | 5.964358581 | 6.023675447 |
| hsa-miR-4324_st | 7.468077064 | 7.371011277 | 4.226813577 | 6.338509284 |
| hsa-miR-4256_st | 3.840062511 | 3.92147895 | 3.992051935 | 3.833036528 |
| hsa-miR-4257_st | 11.85072847 | 12.07146525 | 12.95533475 | 13.20619486 |
| hsa-miR-4258_st | 7.835951648 | 7.521094447 | 7.774478173 | 8.196121982 |
| hsa-miR-4259_st | 11.22028681 | 10.89034974 | 11.28400256 | 11.89975642 |
| hsa-miR-4260_st | 10.22753334 | 10.3305395 | 11.33693614 | 11.48469591 |
| hsa-miR-4253_st | 11.27381284 | 10.94377554 | 11.78299111 | 12.01375435 |
| hsa-miR-4251_st | 5.247752931 | 5.834816805 | 6.319884071 | 6.535681026 |
| hsa-miR-4254_st | 5.575751709 | 5.512302837 | 5.309655955 | 4.667917989 |
| hsa-miR-4255_st | 3.113345185 | 2.381933512 | 1.366451826 | 7.557792937 |
| hsa-miR-4252_st | 4.642329613 | 4.601515033 | 4.905025678 | 4.684139082 |
| hsa-miR-4325_st | 3.2248685 | 2.812017108 | 3.168209875 | 2.256222273 |
| hsa-miR-4326_st | 4.969969258 | 4.172804398 | 6.257555083 | 5.277796448 |
| hsa-miR-4327_st | 12.2435562 | 11.91994721 | 12.43537573 | 12.55450382 |
| hsa-miR-4261_st | 8.061055429 | 8.581054432 | 8.833331508 | 8.488958353 |
| hsa-miR-4265_st | 6.867547783 | 7.464642575 | 7.971004655 | 8.097834643 |
| hsa-miR-4266_st | 5.978349239 | 6.120270507 | 6.524133334 | 6.919127356 |
| hsa-miR-4267_st | 5.089888506 | 5.721675396 | 5.671919771 | 6.112771819 |
| hsa-miR-4262_st | 1.068759287 | 1.645914408 | 2.98917695 | 1.422560709 |
| hsa-miR-2355_st | 1.770925776 | 1.983582634 | 2.015509587 | 2.910491525 |
| hsa-miR-4268_st | 3.715547239 | 3.541068169 | 5.509252136 | 3.925135426 |
| hsa-miR-4269_st | 7.787224879 | 7.416545045 | 7.973918336 | 8.119422079 |
| hsa-miR-4263_st | 2.142752819 | 2.486840227 | 2.278193495 | 1.306221794 |
| hsa-miR-4264_st | 2.068394171 | 1.980206287 | 2.207760671 | 2.626465906 |
| hsa-miR-4270_st | 15.86093217 | 15.0138598 | 15.15773455 | 15.88409745 |
| hsa-miR-4271_st | 11.64984361 | 11.37047897 | 12.05306749 | 12.4654521 |
| hsa-miR-4272_st | 1.71058204 | 0.790256489 | 1.16267958 | 1.535365445 |
| hsa-miR-4273_st | 1.207960197 | 1.453436788 | 0.406491662 | 0.070274307 |
| hsa-miR-4276_st | 1.244872957 | 0.905032176 | 0.753387042 | 0.631445531 |
| hsa-miR-4275_st | 1.743568249 | 1.941266577 | 2.485463958 | 0.979681498 |
| hsa-miR-4274_st | 6.019798539 | 5.355152175 | 4.971018592 | 5.803213885 |
| hsa-miR-4281_st | 16.43866443 | 15.3682291 | 15.33657972 | 16.23080115 |
| hsa-miR-4277_st | 2.397083326 | 2.716033187 | 2.586332016 | 1.94229493 |
| hsa-miR-4279_st | 5.357090115 | 5.662692271 | 6.536614389 | 5.766521327 |
| hsa-miR-4278_st | 6.270251624 | 6.807727643 | 7.677343221 | 8.285024817 |
| hsa-miR-4280_st | 4.016164407 | 4.402783235 | 3.886184729 | 4.003929803 |
| hsa-miR-4282_st | −0.03850547 | −0.041624091 | 0.454318683 | 0.156785332 |
| hsa-miR-4285_st | 11.85139308 | 11.34823563 | 12.6224675 | 13.04732808 |
| hsa-miR-4283_st | 8.636403456 | 8.544465088 | 9.289331874 | 9.443886953 |
| hsa-miR-4234_st | 14.82958188 | 14.57174313 | 15.33414685 | 15.01590166 |
| hsa-miR-4286_st | 13.40774722 | 13.92185021 | 13.50431208 | 14.35078797 |
| hsa-miR-4287_st | 4.69985764 | 4.678911609 | 5.40942507 | 5.086449512 |
| hsa-miR-4288_st | 4.028529409 | 3.984581289 | 3.54474729 | 3.082570632 |
| hsa-miR-4292_st | 7.214290498 | 6.944202264 | 7.544399934 | 7.490346603 |
| hsa-miR-4289_st | 4.905355479 | 6.179714771 | 6.959824746 | 5.190720705 |
| hsa-miR-4290_st | 5.899223832 | 7.215696878 | 6.951214183 | 6.947467062 |
| hsa-miR-4291_st | 4.843792255 | 6.080465464 | 5.937078493 | 5.725932852 |
| hsa-miR-4329_st | 6.357568907 | 5.882436315 | 4.44305889 | 5.193260045 |
| hsa-miR-4330_st | 4.621260415 | 5.870757673 | 6.668679549 | 6.409822521 |
| hsa-miR-500b_st | 3.626476586 | 3.978498443 | 5.124247944 | 3.703260432 |
| hsa-miR-4328_st | 2.920160349 | 3.329034389 | 3.251905407 | 0.818476267 |

TABLE 4B
normalized array data for the 55 benign and malignant FFPE samples.
Average Expression

| | HASH | PTC | FVPTC | FTC | OFTC | ATC | MED |
|---|---|---|---|---|---|---|---|
| miRNA | (Mean) | (Mean) | (Mean) | (Mean) | (Mean) | (Mean) | (Mean) |
| hsa-let-7a_st | 14.15558717 | 14.59021467 | 14.79633591 | 15.05698319 | 12.67273374 | 13.66442503 | 14.22234562 |
| hsa-let-7a-star_st | 2.000910793 | 2.272276021 | 2.12122997 | 2.048872311 | 1.767978037 | 1.558502008 | 2.059719333 |
| hsa-let-7a-2-star_st | 5.168356321 | 4.61680414 | 5.286619394 | 4.872932612 | 5.293124276 | 4.549405249 | 4.518435998 |
| hsa-let-7b_st | 13.27317996 | 13.58463025 | 14.28040197 | 14.08870415 | 11.74870694 | 12.66805825 | 13.59467828 |
| hsa-let-7b-star_st | 6.817059455 | 6.069718412 | 6.30137353 | 5.666949977 | 6.373685054 | 6.99973529 | 6.382344652 |
| hsa-let-7c_st | 13.84045117 | 14.25013036 | 14.72490417 | 14.77839155 | 12.06631017 | 13.17694019 | 14.03440391 |
| hsa-let-7c-star_st | 3.152252428 | 3.83595483 | 3.915512562 | 4.160489257 | 4.442004088 | 4.448860523 | 4.159266112 |
| hsa-let-7d_st | 13.49512033 | 14.07276021 | 14.56294519 | 14.71702284 | 12.37431602 | 13.16709374 | 13.78320506 |
| hsa-let-7d-star_st | 7.719150742 | 7.842045647 | 7.10659991 | 8.149559654 | 7.442085512 | 7.293443086 | 7.660955046 |
| hsa-let-7e_st | 11.63534653 | 12.77464894 | 13.22930831 | 13.43136103 | 11.00485748 | 11.27898959 | 12.59794682 |
| hsa-let-7e-star_st | 4.395542061 | 4.237150173 | 4.191062972 | 4.00496397 | 4.85917825 | 3.777219302 | 3.989818735 |
| hsa-let-7f_st | 13.86770986 | 14.17270133 | 14.10332693 | 15.03109521 | 12.48534444 | 13.1700209 | 13.7628527 |
| hsa-let-7f-1-star_st | 4.748353076 | 3.240654007 | 4.196818355 | 3.220476391 | 5.39538426 | 4.544562842 | 4.040318715 |
| hsa-let-7f-2-star_st | 2.642786315 | 2.24369218 | 2.66054664 | 1.403793951 | 2.187887944 | 2.453876072 | 1.765776531 |
| hsa-miR-15a_st | 10.22855261 | 10.78554572 | 11.78138922 | 11.61137172 | 9.208379685 | 9.55795377 | 10.03911845 |
| hsa-miR-15a-star_st | 4.537517946 | 4.23094415 | 3.790423445 | 4.078921743 | 5.221031665 | 3.371420821 | 4.389824211 |
| hsa-miR-16_st | 13.67601185 | 13.91398442 | 14.45033516 | 14.30677628 | 11.56732264 | 13.41227708 | 13.35525998 |
| hsa-miR-16-1-star_st | 3.567191861 | 3.49373081 | 3.934272778 | 3.874926103 | 3.149129864 | 4.565050367 | 3.279612644 |
| hsa-miR-17_st | 10.33571266 | 10.35543015 | 11.70579728 | 11.17207422 | 9.350341962 | 9.497373109 | 8.976596477 |
| hsa-miR-17-star_st | 5.533789427 | 5.802410675 | 6.615134745 | 6.244423759 | 6.168633685 | 5.627433445 | 3.308338717 |
| hsa-miR-18a_st | 5.002356167 | 4.78929286 | 5.820447886 | 5.5881992 | 5.623384578 | 4.647298608 | 4.119516835 |
| hsa-miR-18a-star_st | 4.745056518 | 4.881361159 | 4.71148011 | 4.768974009 | 5.363608716 | 5.512723702 | 4.103737228 |
| hsa-miR-19a-star_st | 0.898433319 | 0.978313376 | 0.337970327 | 0.16307698 | 0.63379919 | 0.105735692 | 0.412517604 |
| hsa-miR-19a_st | 5.545129704 | 4.92264926 | 6.730049896 | 6.990245501 | 4.719046431 | 3.523181402 | 1.85946981 |
| hsa-miR-19b-1-star_st | 2.075057342 | 2.915718065 | 3.862448035 | 2.471369829 | 2.870994001 | 1.613601082 | 1.049024525 |
| hsa-miR-19b_st | 10.15135781 | 9.874982906 | 10.69568346 | 11.23338678 | 9.37194437 | 8.973546187 | 7.655901885 |
| hsa-miR-19b-2-star_st | 1.267334379 | 1.345956033 | 1.998329834 | −0.141021071 | 1.530205954 | 0.489036019 | 0.602051649 |
| hsa-miR-20a_st | 9.842580341 | 9.209579827 | 11.18583966 | 10.70485318 | 9.310415945 | 8.800586904 | 8.429350381 |
| hsa-miR-20a-star_st | 4.44557718 | 7.677528358 | 4.30644141 | 5.227305497 | 6.9900236 | 2.48889582 | 6.566144449 |
| hsa-miR-21_st | 11.54698196 | 13.04009013 | 11.98964111 | 13.11796716 | 11.10399051 | 13.87368972 | 12.05333621 |
| hsa-miR-21-star_st | 6.30420299 | 6.778416463 | 6.316638798 | 6.5515016 | 6.631277332 | 7.976329149 | 5.735722094 |
| hsa-miR-22-star_st | 6.421952845 | 6.789741956 | 8.524052209 | 9.106625368 | 8.151159519 | 6.617810833 | 7.31101491 |
| hsa-miR-22_st | 11.05277738 | 11.44987129 | 13.27065348 | 13.13256133 | 11.24763135 | 11.9823142 | 11.28321014 |
| hsa-miR-23a-star_st | 10.31104907 | 9.858473752 | 9.903093535 | 10.01753639 | 10.73847369 | 10.93500291 | 10.65124107 |
| hsa-miR-23a_st | 13.5963321 | 14.53458654 | 14.78480566 | 14.8950786 | 12.43065282 | 14.20246249 | 14.76056881 |
| hsa-miR-24-1-star_st | 2.890137011 | 3.273428365 | 5.168339476 | 5.737672979 | 3.566322415 | 2.189673162 | 5.165794755 |
| hsa-miR-24_st | 12.74765201 | 13.68250263 | 14.28444905 | 14.21881702 | 11.7794624 | 13.14715514 | 13.75024265 |
| hsa-miR-24-2-star_st | 4.932204015 | 5.746042115 | 6.81998025 | 7.530642489 | 4.675881831 | 6.852119208 | 5.301711427 |
| hsa-miR-25-star_st | 12.1346603 | 11.04052605 | 11.43680338 | 11.14512285 | 12.04988373 | 12.89759501 | 12.34140131 |
| hsa-miR-25_st | 10.39185859 | 10.10631951 | 11.36722724 | 11.67144628 | 9.181655345 | 10.43144687 | 10.25257266 |
| hsa-miR-26a_st | 14.77205274 | 14.9925562 | 14.29109135 | 15.36110683 | 12.82152635 | 14.22653157 | 14.35755585 |
| hsa-miR-26a-1-star_st | 1.426595703 | 1.395898206 | 1.375607255 | 1.583961486 | 2.158652188 | 1.15896614 | 1.574951234 |
| hsa-miR-26b_st | 11.45304815 | 11.73506896 | 11.71530952 | 13.57376249 | 10.27168874 | 10.19870097 | 11.87214728 |
| hsa-miR-26b-star_st | 4.799189993 | 4.863637795 | 5.860061359 | 6.363199271 | 4.169823611 | 4.235954019 | 5.119257805 |
| hsa-miR-27a-star_st | 5.170785201 | 5.480304052 | 4.926467582 | 5.935919287 | 5.764092638 | 5.474807064 | 4.366513168 |
| hsa-miR-27a_st | 11.83395926 | 12.70262712 | 12.91788995 | 13.56781561 | 10.81134798 | 12.05985028 | 12.54097106 |
| hsa-miR-28-5p_st | 7.926705601 | 8.039287499 | 9.87504255 | 9.903931374 | 6.804963959 | 8.362124195 | 8.654979082 |
| hsa-miR-28-3p_st | 6.47522152 | 6.384773469 | 6.772715323 | 7.239416061 | 5.003913242 | 7.516807855 | 6.898328183 |
| hsa-miR-29a-star_st | 1.236542813 | 2.427204244 | 1.993710542 | 1.138996668 | 2.941554319 | 1.406089468 | 0.339519955 |
| hsa-miR-29a_st | 13.1147982 | 13.78672265 | 13.91974692 | 14.64068283 | 12.44553549 | 12.94341798 | 13.38369759 |
| hsa-miR-30a_st | 11.82133057 | 12.09619493 | 13.49493147 | 13.81781814 | 11.01569113 | 10.35405371 | 11.7092504 |
| hsa-miR-30a-star_st | 9.397240205 | 8.690107102 | 10.71191557 | 11.3324932 | 8.806171869 | 6.644641364 | 7.522593943 |
| hsa-miR-31_st | 9.93589464 | 11.66755968 | 10.0728959 | 10.77522615 | 6.617768559 | 11.07542167 | 6.899861768 |
| hsa-miR-31-star_st | 4.482548351 | 5.822449532 | 4.962247496 | 5.929216999 | 1.021173127 | 4.838256477 | 2.0223309 |
| hsa-miR-32_st | 0.353603165 | 2.062220233 | 0.768164548 | 2.194132191 | 1.041383646 | 1.155493738 | 0.341168283 |
| hsa-miR-32-star_st | 2.99947611 | 3.151384348 | 3.834688154 | 3.406030872 | 1.409075005 | 1.329716854 | 2.431588363 |
| hsa-miR-33a_st | −0.711921589 | 0.893086155 | 1.185216905 | 1.104654008 | 0.721154112 | 0.2920118 | −0.42424147 |
| hsa-miR-33a-star_st | 3.124682244 | 2.767795205 | 3.930904133 | 5.591248043 | 2.837470909 | 1.893753394 | 2.109337512 |
| hsa-miR-92a-1-star_st | 7.203125347 | 6.804136576 | 7.289368022 | 7.986137877 | 7.243504228 | 8.061767379 | 8.293312403 |
| hsa-miR-92a_st | 10.36678173 | 11.22137732 | 11.45815148 | 11.23863627 | 9.378182258 | 10.26781596 | 9.958756909 |
| hsa-miR-92a-2-star_st | 8.419567696 | 8.023478386 | 8.333560558 | 8.508241911 | 8.366199316 | 9.263381064 | 9.154700975 |
| hsa-miR-93_st | 9.381962353 | 9.51784926 | 11.57735451 | 10.82438539 | 8.515727457 | 9.637784101 | 9.288556963 |
| hsa-miR-93-star_st | 4.823343886 | 4.674976822 | 5.294583512 | 5.261968088 | 5.029624933 | 4.586652533 | 5.518594839 |
| hsa-miR-95_st | 6.605124298 | 6.582011528 | 8.159989018 | 9.566997361 | 6.047061768 | 4.577933434 | 8.147387044 |
| hsa-miR-96_st | 4.06747702 | 5.665218456 | 7.302927712 | 7.881283843 | 5.558531727 | 3.591208411 | 6.495520721 |
| hsa-miR-96-star_st | 3.23829881 | 3.183461799 | 3.757992978 | 3.665806318 | 2.742345389 | 1.921073934 | 3.036074318 |
| hsa-miR-98_st | 8.764035515 | 9.582128348 | 11.28216671 | 11.19300317 | 9.248880372 | 8.491389814 | 9.806015732 |
| hsa-miR-99a_st | 12.12833834 | 12.52868936 | 13.32185912 | 13.12940031 | 10.98598063 | 11.27890857 | 12.79287611 |
| hsa-miR-99a-star_st | 3.920141689 | 4.695537102 | 5.540634899 | 5.095796428 | 6.034168337 | 3.969663981 | 5.153626375 |
| hsa-miR-100_st | 12.06947437 | 11.95528815 | 12.93969011 | 13.02190847 | 10.9314438 | 11.9555963 | 12.39840916 |
| hsa-miR-100-star_st | 1.868638372 | 2.040636571 | 1.60575921 | 1.730884757 | 2.189441974 | 2.076183724 | 1.735104117 |
| hsa-miR-101-star_st | 2.774201676 | 2.517878954 | 3.441190905 | 2.356353358 | 2.957629665 | 1.558055369 | 1.599150544 |
| hsa-miR-101_st | 5.192614949 | 6.207942029 | 6.982511176 | 6.619860027 | 4.414594501 | 3.516447501 | 4.589175546 |
| hsa-miR-29b-1-star_st | 6.202272412 | 5.867594055 | 7.076130015 | 6.770624046 | 6.314643455 | 5.417821018 | 5.292955967 |
| hsa-miR-29b_st | 11.17914318 | 12.2967625 | 11.75861177 | 13.30816913 | 10.29315732 | 10.56349555 | 11.13933078 |
| hsa-miR-29b-2-star_st | 7.031637888 | 7.226713588 | 8.740549041 | 9.610294833 | 6.801455244 | 6.200969235 | 8.267482535 |
| hsa-miR-103-2-star_st | 5.214095015 | 4.949109865 | 5.356796976 | 5.41040604 | 4.933801977 | 5.241155419 | 4.919688613 |
| hsa-miR-103_st | 11.88316092 | 12.43837453 | 13.76870378 | 13.67006857 | 11.36550876 | 11.69697438 | 12.00691348 |
| hsa-miR-105_st | 2.733230098 | 2.473734812 | 1.878935221 | 1.569650397 | 2.584630708 | 1.633457684 | 1.438569598 |
| hsa-miR-105-star_st | 2.910784228 | 2.304464764 | 2.620903285 | 3.303375162 | 3.221779591 | 2.94482592 | 2.845816659 |
| hsa-miR-106a_st | 10.34722345 | 10.38103063 | 11.84159746 | 11.09438953 | 9.25435793 | 9.487342321 | 8.930793388 |
| hsa-miR-106a-star_st | 1.362472709 | 2.290006472 | 0.570145726 | 1.511380533 | 0.224515551 | 0.491056474 | 1.62560452 |
| hsa-miR-107_st | 11.69556531 | 12.36038512 | 13.57948492 | 13.53198517 | 10.7912168 | 11.59297499 | 12.07227501 |
| hsa-miR-16-2-star_st | 3.973489139 | 4.087432032 | 4.530458716 | 3.788449282 | 4.9965682 | 2.320337009 | 4.520236468 |
| hsa-miR-192_st | 7.492753339 | 7.058302068 | 8.909646011 | 9.130219767 | 6.803319762 | 5.532400548 | 6.009148637 |
| hsa-miR-192-star_st | 3.418696746 | 4.151977557 | 4.023693518 | 4.83942338 | 3.103107233 | 2.796057305 | 3.101445368 |
| hsa-miR-196a_st | 2.222208824 | 3.378319401 | 3.57719364 | 3.288017833 | 3.398751492 | 2.490453722 | 2.870212301 |
| hsa-miR-197_st | 7.738885553 | 7.215557733 | 6.425273481 | 7.788989972 | 7.176391256 | 7.216398895 | 6.904313002 |
| hsa-miR-198_st | 11.8334179 | 11.4220187 | 11.31439721 | 12.01765106 | 11.7956482 | 12.63872181 | 12.59586918 |
| hsa-miR-199a-5p_st | 10.09721053 | 9.449549306 | 10.77755948 | 10.59055768 | 7.437834534 | 11.27821785 | 9.908600555 |
| hsa-miR-199a-3p_st | 11.5616895 | 10.58923267 | 11.97851807 | 12.35393875 | 8.250820644 | 12.34931903 | 11.44429994 |
| hsa-miR-208a_st | 2.044248201 | 2.942199916 | 2.949751166 | 2.392810973 | 3.123211201 | 2.529824133 | 3.122450383 |
| hsa-miR-129-5p_st | 10.75875476 | 10.2285354 | 9.989510104 | 10.86639337 | 11.0719225 | 11.81694159 | 11.53393392 |
| hsa-miR-129-star_st | 4.435826896 | 4.456336479 | 3.393844283 | 4.703448924 | 3.909189517 | 4.093172285 | 5.017997908 |
| hsa-miR-148a-star_st | 4.630457714 | 4.545521342 | 4.398764816 | 5.424393108 | 4.405005014 | 4.609467366 | 4.350362905 |
| hsa-miR-148a_st | 10.16047549 | 10.50327901 | 11.47098471 | 12.31219272 | 9.659307219 | 9.746201307 | 9.639611137 |
| hsa-miR-30c_st | 12.9739315 | 13.20297343 | 13.61263571 | 14.56123872 | 12.03154511 | 11.56601999 | 12.90391769 |
| hsa-miR-30c-2-star_st | 7.273820491 | 7.233754822 | 7.269872796 | 7.133733724 | 7.69512299 | 6.553367089 | 6.810625105 |
| hsa-miR-30d_st | 11.53951636 | 12.26949304 | 13.33428583 | 13.25323129 | 10.58560508 | 10.73618037 | 12.21827765 |
| hsa-miR-30d-star_st | 2.659001626 | 2.266437028 | 3.558440047 | 3.560641096 | 3.253253053 | 0.656983056 | 1.415498987 |
| hsa-miR-139-5p_st | 6.991887904 | 6.188167636 | 7.056761228 | 6.771251705 | 4.672489331 | 5.433336135 | 4.28007157 |
| hsa-miR-139-3p_st | 7.497881274 | 6.718077082 | 7.020533496 | 6.844999487 | 7.737523062 | 7.683109716 | 7.597040698 |
| hsa-miR-147_st | 4.286284966 | 4.231462063 | 3.596554731 | 4.051340218 | 4.449678211 | 3.523859523 | 3.60707822 |
| hsa-miR-7_st | 6.829008756 | 4.76153663 | 8.711389161 | 7.015778387 | 5.678030047 | 3.265153284 | 10.09715629 |
| hsa-miR-7-1-star_st | 6.128291711 | 5.775823686 | 6.929382687 | 6.360395059 | 5.839142228 | 5.920844784 | 7.449272665 |
| hsa-miR-7-2-star_st | 5.152212561 | 5.416573335 | 7.230029186 | 6.318720515 | 6.069846332 | 4.282075592 | 5.113401839 |
| hsa-miR-10a_st | 7.817762825 | 7.543722306 | 9.127189043 | 8.269499463 | 6.218609015 | 8.297859122 | 10.92463042 |
| hsa-miR-10a-star_st | 3.823003487 | 3.559641377 | 4.128900891 | 3.36731792 | 4.512958902 | 4.450666032 | 5.913660357 |
| hsa-miR-10b_st | 8.974220983 | 9.213328465 | 9.506927579 | 10.1234253 | 9.529424914 | 9.34901616 | 9.955072789 |
| hsa-miR-10b-star_st | 3.06527396 | 3.99684384 | 3.395801858 | 3.709203984 | 4.634592662 | 3.730818264 | 4.327067157 |
| hsa-miR-34 a_st | 8.633908516 | 10.03888339 | 11.81293507 | 11.03795179 | 9.419917206 | 9.753800163 | 9.170093443 |
| hsa-miR-34a-star_st | 5.014888394 | 6.53248197 | 6.390812722 | 7.473209474 | 6.567990586 | 6.34249384 | 5.782483676 |
| hsa-miR-181a_st | 10.96798371 | 12.653882 | 13.0799677 | 14.10751045 | 9.455426499 | 11.95980569 | 11.27802999 |
| hsa-miR-181a-2-star_st | 7.330540203 | 8.784836445 | 10.23210011 | 10.63227287 | 6.481159895 | 7.338199195 | 6.465805715 |
| hsa-miR-181b_st | 9.767864295 | 11.35343817 | 11.85274514 | 12.80521069 | 8.714115373 | 10.70360943 | 9.938603195 |
| hsa-miR-181c_st | 6.827420887 | 9.144540298 | 9.513495627 | 10.4666294 | 5.910773463 | 8.254549086 | 7.645745719 |
| hsa-miR-181c-star_st | 6.49754933 | 6.254712519 | 7.474375122 | 6.292237196 | 6.769313803 | 7.371440703 | 6.323862707 |
| hsa-miR-182_st | 6.677081403 | 8.885071506 | 10.61688438 | 10.28127884 | 8.102190578 | 6.680846331 | 10.37933166 |
| hsa-miR-182-star_st | 2.012069982 | 1.919635521 | 1.501307095 | 0.944835915 | 1.867009587 | 1.854240768 | 1.857618328 |
| hsa-miR-183_st | 3.766386098 | 5.360403714 | 5.511571562 | 7.123350479 | 5.125361201 | 4.317192692 | 7.944247235 |
| hsa-miR-183-star_st | 6.89440698 | 6.410652784 | 6.091296487 | 6.550299392 | 6.916808505 | 7.601743634 | 7.239483546 |
| hsa-miR-187-star_st | 9.526590037 | 8.775800551 | 8.581223968 | 8.884449193 | 9.637912093 | 10.13708744 | 9.730203101 |
| hsa-miR-187_st | 4.672892852 | 4.092445298 | 4.309999443 | 4.105007888 | 5.142451974 | 4.310364957 | 2.923682593 |
| hsa-miR-196a-star_st | 4.36952091 | 4.67877903 | 4.781823853 | 5.047647902 | 5.718324409 | 4.771580955 | 3.725223479 |
| hsa-miR-199b-5p_st | 7.593858521 | 5.598570435 | 7.292452208 | 7.89141558 | 4.37259123 | 9.015192133 | 6.04669207 |
| hsa-miR-199b-3p_st | 11.41795063 | 11.06442715 | 11.84681756 | 12.20388891 | 8.031814669 | 12.27579123 | 11.33329201 |
| hsa-miR-203_st | 5.245024372 | 7.151890717 | 7.504746081 | 6.2452748 | 7.477416154 | 5.851978878 | 3.458763043 |
| hsa-miR-204_st | 5.663648348 | 5.664581417 | 7.82831927 | 7.233124112 | 5.142731169 | 1.986843962 | 5.022496021 |
| hsa-miR-205_st | 5.335849885 | 4.28878994 | 4.599205678 | 5.028069709 | 2.267075636 | 8.308836448 | 1.218782129 |
| hsa-miR-205-star_st | 5.445961267 | 4.606051166 | 5.154179779 | 4.395409233 | 5.284450763 | 4.248301802 | 5.633643845 |
| hsa-miR-210_st | 11.84418496 | 11.78697555 | 11.45186249 | 12.16162053 | 12.10456496 | 12.88926558 | 12.70932084 |
| hsa-miR-211_st | 6.339653367 | 4.628308407 | 5.085480393 | 3.795096046 | 6.5372267 | 5.081828594 | 5.143133761 |
| hsa-miR-212_st | 5.976116621 | 6.158293546 | 6.370343852 | 5.523670347 | 6.798856131 | 6.479408109 | 6.007727052 |
| hsa-miR-181a-star_st | 4.357356256 | 5.466988472 | 5.892538151 | 7.643065661 | 4.121950974 | 5.861952622 | 5.253225189 |
| hsa-miR-214-star_st | 5.088593454 | 4.594336315 | 5.816660233 | 5.046719015 | 4.460684971 | 6.018471611 | 6.2945065 |
| hsa-miR-214_st | 10.62938533 | 9.658784309 | 10.44747637 | 9.582368563 | 10.19162672 | 10.71961042 | 9.901004022 |
| hsa-miR-215_st | 4.270591269 | 4.311806864 | 5.098956407 | 5.485850806 | 4.801810688 | 3.617281012 | 3.941171568 |
| hsa-miR-216a_st | 1.883856723 | 2.286469844 | 1.42625946 | 3.822753636 | 3.952866256 | 1.73311264 | 1.757123104 |
| hsa-miR-217_st | 1.695666778 | 3.035235214 | 2.301735044 | 2.412148892 | 3.240006341 | 2.49636166 | 2.038495932 |
| hsa-miR-218_st | 3.507417369 | 4.738090632 | 5.880315143 | 6.813675371 | 3.670812429 | 5.136511661 | 2.555380674 |
| hsa-miR-218-1-star_st | 1.80837225 | 1.631812146 | 0.767883102 | 1.115131244 | 2.462172456 | 2.233346376 | 1.476511475 |
| hsa-miR-218-2-star_st | 3.460035795 | 3.457361177 | 3.011797663 | 2.055196392 | 3.609866248 | 2.735933066 | 2.005229681 |
| hsa-miR-219-5p_st | 0.558006232 | 0.726226699 | 1.215404486 | −0.02016011 | 1.479172476 | −0.343115386 | 0.18328327 |
| hsa-miR-219-1-3p_st | 4.381347765 | 4.477786371 | 4.425702516 | 4.951708927 | 4.707411535 | 5.034158813 | 4.379737187 |
| hsa-miR-220a_st | 2.087789184 | 2.425674514 | 2.357882657 | 1.538952393 | 1.676918204 | 1.647878646 | 2.501871406 |
| hsa-miR-221-star_st | 6.066776382 | 8.563593398 | 9.559644886 | 6.630949794 | 6.979899544 | 7.203735635 | 7.089821709 |
| hsa-miR-221_st | 12.13001047 | 14.57854186 | 14.26153434 | 12.53174497 | 11.64481607 | 13.89436439 | 13.27234191 |
| hsa-miR-222-star_st | 5.818279578 | 7.132332433 | 7.589451472 | 5.531120456 | 6.541231848 | 5.708590479 | 4.920460405 |
| hsa-miR-222_st | 11.98922523 | 14.51881613 | 13.96555929 | 12.41445009 | 11.58376583 | 13.59523865 | 12.86215637 |
| hsa-miR-223-star_st | 3.163623433 | 3.358138524 | 2.774117573 | 3.457772071 | 2.444782519 | 4.449474443 | 2.491377585 |
| hsa-miR-223_st | 9.650721078 | 8.603195453 | 8.375693368 | 10.66239316 | 8.552718061 | 10.80554703 | 8.168209329 |
| hsa-miR-224_st | 5.064450025 | 6.411892369 | 7.704669542 | 8.739896628 | 6.750249122 | 7.249377988 | 6.885702763 |
| hsa-miR-224-star_st | 4.507544545 | 5.064934247 | 5.929024663 | 6.906067197 | 5.280013536 | 4.859059332 | 5.030770951 |
| hsa-miR-200b-star_st | 6.105280153 | 6.258765338 | 5.611969958 | 6.062586469 | 5.754332265 | 5.515944588 | 6.652708038 |
| hsa-miR-200b_st | 10.60880916 | 11.58154047 | 10.62678477 | 12.60697709 | 7.14146164 | 9.065166932 | 11.11433327 |
| hsa-let-7g_st | 13.18726298 | 13.34889669 | 13.82042491 | 14.78719799 | 11.67193933 | 12.65946324 | 12.83057161 |
| hsa-let-7g-star_st | 4.529559879 | 4.591773812 | 5.02643075 | 4.415556107 | 5.519437669 | 5.28765056 | 4.648485709 |
| hsa-let-7i_st | 13.32603748 | 14.0670837 | 14.79153195 | 15.03012353 | 11.66674839 | 13.38627063 | 12.74185859 |
| hsa-let-7i-star_st | 5.994641236 | 6.187109219 | 6.557306317 | 5.853826111 | 5.755943505 | 6.20908114 | 5.45977697 |
| hsa-miR-1_st | 2.895608646 | 2.354832255 | 5.688104289 | 4.810969245 | 3.377879698 | 2.66589044 | 4.564563909 |
| hsa-miR-15b_st | 10.10475455 | 10.60944185 | 10.99276936 | 11.39267049 | 8.616058957 | 9.222821702 | 9.753888403 |
| hsa-miR-15b-star_st | 4.141388282 | 1.82713164 | 2.592140593 | 3.264764547 | 4.037068329 | 2.504335849 | 2.981822737 |
| hsa-miR-23b-star_st | 5.341773863 | 5.381732117 | 5.073605297 | 5.482529408 | 4.939352772 | 4.560027664 | 5.249563316 |
| hsa-miR-23b_st | 13.78050337 | 14.63961365 | 14.7121197 | 15.07621511 | 12.25217307 | 13.94532136 | 15.12780576 |
| hsa-miR-27b-star_st | 5.899655559 | 6.447717923 | 7.816555897 | 7.492233784 | 6.693311721 | 5.199909202 | 6.907610254 |
| hsa-miR-27b_st | 11.70151257 | 12.41039491 | 13.34233946 | 13.99454949 | 10.82641477 | 11.23271974 | 12.80217355 |
| hsa-miR-30b_st | 12.04580773 | 12.4872028 | 13.10948587 | 13.78897012 | 10.56878692 | 10.41817895 | 11.8810397 |
| hsa-miR-30b-star_st | 8.76947302 | 7.781993417 | 8.118760757 | 8.094038443 | 9.319870737 | 9.002742118 | 9.07655031 |
| hsa-miR-122_st | 3.585326852 | 2.737253917 | 3.446951702 | 3.027338387 | 4.430544251 | 2.375744402 | 3.762012074 |
| hsa-miR-122-star_st | 1.964075286 | 1.947571948 | 1.142933974 | 2.207985365 | 2.351621695 | 1.979414234 | 1.923152294 |
| hsa-miR-124-star_st | 4.794079348 | 5.095088673 | 3.948924275 | 5.135005308 | 4.894830939 | 5.298676745 | 4.750264324 |
| hsa-miR-124_st | 7.806511128 | 7.784305524 | 7.07705244 | 7.397269122 | 6.743404889 | 8.627227002 | 8.925469795 |
| hsa-miR-125b_st | 14.1919019 | 14.97554962 | 14.42866721 | 15.29685476 | 12.40493896 | 13.731989 | 14.83731351 |
| hsa-miR-125b-1-star_st | 11.36946131 | 11.16199369 | 10.49754353 | 10.83320229 | 11.62693935 | 12.26047773 | 11.90564221 |
| hsa-miR-128_st | 7.422848484 | 7.216004634 | 9.276139556 | 9.140042075 | 6.32007373 | 7.407939845 | 8.333243989 |
| hsa-miR-130a-star_st | 0.786479804 | 2.10985463 | 1.544702029 | 0.631679791 | 1.451754806 | 0.991927412 | 0.787444139 |
| hsa-miR-130a_st | 10.95251849 | 11.3280284 | 12.77911769 | 12.79495895 | 9.652723919 | 9.584293959 | 9.26605896 |
| hsa-miR-132-star_st | 1.741616958 | 2.682784372 | 4.012137023 | 3.684869004 | 3.167044926 | 3.247791097 | 4.216315171 |
| hsa-miR-132_st | 6.784279235 | 7.041617861 | 7.180649067 | 7.613218152 | 4.23808657 | 7.382487951 | 8.819555589 |
| hsa-miR-133a_st | 4.021975192 | 4.04792149 | 5.808665815 | 5.20248349 | 5.052777918 | 3.559910657 | 5.077578674 |
| hsa-miR-135a_st | 1.067953188 | 1.748144202 | 4.43473341 | 4.308808333 | 1.950107949 | 0.317751258 | 1.116647141 |
| hsa-miR-135a-star_st | 11.83549917 | 11.33078949 | 11.11368328 | 11.85564522 | 11.92740925 | 12.59260092 | 12.38596622 |
| hsa-miR-137_st | 2.001380579 | 1.815176614 | 1.392432866 | 1.364279415 | 0.825130406 | 1.751735688 | 0.982315307 |
| hsa-miR-138_st | 10.42071623 | 9.974431864 | 12.2247727 | 10.17848543 | 9.684958709 | 7.777501433 | 8.895437529 |
| hsa-miR-138-2-star_st | 4.681402799 | 4.037846732 | 4.139818818 | 4.195616795 | 5.043503354 | 3.500263107 | 4.116858746 |
| hsa-miR-140-5p_st | 4.280632595 | 4.484409805 | 5.938300771 | 7.505254469 | 4.415534157 | 4.585066748 | 5.856707796 |
| hsa-miR-140-3p_st | 11.21159969 | 10.34538914 | 11.6177264 | 11.49546455 | 9.837357601 | 10.58542797 | 10.54373498 |
| hsa-miR-141-star_st | 1.764082774 | 1.60780637 | 3.762955058 | 3.166838758 | 3.970558121 | 0.927821235 | 1.071939233 |
| hsa-miR-141_st | 8.246432824 | 9.722639288 | 10.5589574 | 10.87221424 | 8.489656305 | 6.001322071 | 6.726696143 |
| hsa-miR-142-5p_st | 7.697000272 | 4.46124026 | 4.251002986 | 4.771781888 | 5.545056055 | 6.609062526 | 3.020451748 |
| hsa-miR-142-3p_st | 1.479133811 | 1.627178642 | 0.558456298 | −0.236153373 | 1.150118252 | 0.605999308 | −0.040190223 |
| hsa-miR-143-star_st | 6.798823037 | 5.997180428 | 7.582575695 | 6.680777283 | 5.598154425 | 6.176383002 | 5.883719468 |
| hsa-miR-143_st | 12.20141256 | 11.65121946 | 13.6549655 | 13.96978701 | 9.839501741 | 11.58629479 | 12.53737939 |
| hsa-miR-144-star_st | 2.289380183 | 3.064744909 | 3.077131071 | 2.995776582 | 1.409755128 | 1.813221703 | 2.269920652 |
| hsa-miR-144_st | 1.033913323 | 1.408971929 | 0.847876762 | 0.321653442 | 0.652074446 | −0.056050181 | 0.524737705 |
| hsa-miR-145_st | 12.75665105 | 12.17800278 | 13.89739783 | 14.40965082 | 10.9061954 | 12.27661388 | 13.98583179 |
| hsa-miR-145-star_st | 4.692695925 | 4.569718396 | 6.199548484 | 7.17489021 | 4.405981305 | 4.699893983 | 5.774812742 |
| hsa-miR-152_st | 9.314848588 | 9.244788583 | 11.67251664 | 11.14921919 | 8.46429467 | 8.882691036 | 8.801029044 |
| hsa-miR-153_st | 1.170922889 | 2.354372267 | 0.91566264 | 1.209452907 | 1.451366337 | 0.92719961 | 1.96295281 |
| hsa-miR-191_st | 12.42525393 | 13.19851021 | 13.62382892 | 13.32984752 | 10.85053591 | 12.23517027 | 13.0339579 |
| hsa-miR-191-star_st | 6.577628988 | 5.530830507 | 5.213814831 | 5.707216115 | 7.181439317 | 6.27655688 | 6.54464126 |
| hsa-miR-9_st | −0.8869132 | 0.224301916 | 0.388235346 | −0.985842924 | 0.137574801 | −0.430840486 | 2.48388758 |
| hsa-miR-9-star_st | 2.194109126 | 2.205316629 | 1.552981658 | 3.599083918 | 0.933166235 | 0.857813509 | 3.304189879 |
| hsa-miR-125a-5p_st | 12.59010926 | 13.88134014 | 13.45615422 | 14.10534867 | 11.44457428 | 12.21721845 | 13.40101043 |
| hsa-miR-125a-3p_st | 11.82467831 | 11.17649043 | 11.27485903 | 11.30233652 | 11.81059014 | 12.06138848 | 12.01479309 |
| hsa-miR-125b-2-star_st | 9.598723423 | 8.351435282 | 8.778905155 | 9.224655821 | 9.533268049 | 10.07319994 | 9.226995842 |
| hsa-miR-126-star_st | 0.480778016 | 0.984734729 | 2.074256246 | 0.919074692 | 1.953659055 | 0.55697652 | 0.564760027 |
| hsa-miR-126_st | 13.40627171 | 12.99389586 | 14.2865301 | 14.7325165 | 11.73948371 | 12.51033892 | 12.09179883 |
| hsa-miR-127-5p_st | 6.065131497 | 5.435765839 | 6.376483893 | 5.215929537 | 7.21081578 | 6.832409463 | 6.671427078 |
| hsa-miR-127-3p_st | 5.688982631 | 5.569513571 | 5.57320905 | 5.22420258 | 6.642025421 | 7.720184782 | 9.678530601 |
| hsa-miR-129-3p_st | 3.147432386 | 3.774912408 | 3.134068451 | 2.363771153 | 3.775266892 | 2.476258704 | 5.931930163 |
| hsa-miR-134_st | 7.357268722 | 7.380168804 | 7.355223284 | 8.278940631 | 8.015559424 | 9.287592102 | 8.619861335 |
| hsa-miR-136_st | 1.681412199 | 0.839714928 | 1.475878647 | 1.86121057 | −0.291764768 | 0.180251077 | 0.020485652 |
| hsa-miR-136-star_st | 1.664595123 | 2.259961149 | 1.79879255 | 1.685114936 | 0.685413677 | 1.306515497 | 0.826916125 |
| hsa-miR-138-1-star_st | 7.369352767 | 6.021438987 | 7.988706123 | 7.335679479 | 6.471158139 | 6.381567728 | 6.703012699 |
| hsa-miR-146a_st | 10.93507161 | 9.636324716 | 10.36910697 | 10.37269315 | 8.261485267 | 10.12460409 | 7.016099341 |
| hsa-miR-146a-star_st | −0.026692432 | 1.645291388 | 1.709677264 | 1.908031509 | 0.385516821 | 1.129266889 | 1.235996692 |
| hsa-miR-149_st | 6.415664237 | 6.662821575 | 6.635770335 | 6.956451277 | 6.599391906 | 6.815466089 | 7.641749729 |
| hsa-miR-149-star_st | 15.08862573 | 15.7012122 | 15.01057966 | 15.43442102 | 14.53262325 | 15.83176583 | 15.66375449 |
| hsa-miR-150_st | 13.1287096 | 9.670581011 | 9.044907059 | 10.43796227 | 8.600444415 | 11.39620093 | 8.450111524 |
| hsa-miR-150-star_st | 12.78897006 | 12.31848817 | 12.11511701 | 12.6453347 | 12.59304601 | 13.86507565 | 13.29326258 |
| hsa-miR-154_st | 4.617459155 | 3.528313533 | 3.324393825 | 3.020937689 | 4.658377149 | 4.141740026 | 5.125460616 |
| hsa-miR-154-star_st | 3.669627148 | 2.91163797 | 3.013163581 | 2.071177464 | 3.32964704 | 3.167627229 | 3.339111144 |
| hsa-miR-184_st | 6.936745513 | 6.622640674 | 6.645739239 | 6.646598493 | 8.497840203 | 7.517968989 | 7.294637147 |
| hsa-miR-185_st | 9.400888787 | 8.881011032 | 10.5448793 | 10.12657698 | 9.606802476 | 8.698018295 | 8.147289208 |
| hsa-miR-185-star_st | 9.434281223 | 7.848030887 | 8.55341008 | 9.045785034 | 9.630778121 | 9.602970198 | 9.696949212 |
| hsa-miR-186_st | 4.395239757 | 3.454877209 | 5.679008137 | 5.783911032 | 3.69175877 | 4.023561427 | 3.841284529 |
| hsa-miR-186-star_st | 0.090880609 | 0.585351444 | 1.042973241 | 2.179470582 | 0.363104408 | 0.637384472 | 0.872261176 |
| hsa-miR-188-5p_st | 6.186688709 | 6.503704553 | 6.573393633 | 6.547511844 | 7.198634804 | 7.688234431 | 7.34307882 |
| hsa-miR-188-3p_st | 3.482262449 | 3.158909902 | 3.858285782 | 3.303776724 | 4.220118015 | 4.047598422 | 3.603964985 |
| hsa-miR-190_st | 0.032419558 | 0.1137425 | −1.217677031 | −0.768260639 | −0.396461744 | 0.084487406 | −0.002141882 |
| hsa-miR-193a-5p_st | 12.00175793 | 11.5281307 | 10.8349368 | 11.3279787 | 11.52244986 | 12.57322299 | 12.31279765 |
| hsa-miR-193a-3p_st | 7.436717114 | 7.166387083 | 8.0179642 | 6.073969598 | 6.290215771 | 7.20796229 | 5.10580256 |
| hsa-miR-194_st | 7.167387408 | 7.251847965 | 8.744947191 | 8.547775573 | 5.909894737 | 5.657501474 | 5.562160525 |
| hsa-miR-195_st | 11.01947046 | 10.70380498 | 12.00806291 | 11.9823632 | 10.38711643 | 10.17200071 | 10.95026168 |
| hsa-miR-195-star_st | 5.312914015 | 4.874648244 | 4.704450833 | 5.83733951 | 5.37893907 | 5.918313564 | 4.750944732 |
| hsa-miR-206_st | 5.326304161 | 5.637537948 | 5.367506599 | 5.238271939 | 6.00097516 | 4.771024315 | 5.694402695 |
| hsa-miR-320a_st | 12.07583982 | 12.03895857 | 12.25696961 | 11.82901769 | 11.43862693 | 12.06035041 | 12.00940815 |
| hsa-miR-200c-star_st | 3.425551521 | 3.183613734 | 4.193372991 | 2.42074175 | 3.310670347 | 2.9424622 | 3.578695497 |
| hsa-miR-200c_st | 12.53647947 | 13.65745284 | 14.40176071 | 14.20922481 | 11.73747464 | 10.76346356 | 12.46358892 |
| hsa-miR-155_st | 11.25575445 | 9.035329102 | 8.985833032 | 9.192711002 | 8.680173678 | 10.73279074 | 7.105913695 |
| hsa-miR-155-star_st | 1.964949002 | 2.467721388 | 2.054812113 | 2.08884536 | 2.023930208 | 1.732035151 | 1.557014646 |
| hsa-miR-194-star_st | 7.69841929 | 7.733534988 | 7.240230495 | 7.181817602 | 8.332122903 | 8.687291268 | 8.002536882 |
| hsa-miR-106b_st | 9.133106521 | 9.379692755 | 11.13695291 | 10.59803448 | 8.280212695 | 8.544609295 | 8.777741762 |
| hsa-miR-106b-star_st | 7.695518233 | 7.518951706 | 7.913585085 | 7.558228849 | 6.264628013 | 7.78163287 | 7.543962549 |
| hsa-miR-29c-star_st | 7.88826684 | 6.769481202 | 8.042632105 | 9.761083082 | 6.934005057 | 5.186342908 | 8.823057821 |
| hsa-miR-29c_st | 11.03910328 | 11.59741823 | 11.54383747 | 13.44223212 | 10.21301967 | 9.626839584 | 11.08211055 |
| hsa-miR-30c-1-star_st | 10.12401426 | 9.636250605 | 10.55657357 | 10.29245385 | 10.85556273 | 10.94996051 | 11.18884027 |
| hsa-miR-200a-star_st | 5.923948898 | 5.84225088 | 5.6910311 | 6.306674155 | 5.818509223 | 6.595250191 | 5.966171141 |
| hsa-miR-200a_st | 9.697820348 | 10.05673761 | 9.748730655 | 11.90218462 | 7.520292358 | 7.599506565 | 9.883965305 |
| hsa-miR-302a-star_st | 3.051030388 | 2.231881494 | 2.126364102 | 2.658996485 | 3.099562106 | 2.729499945 | 1.98996486 |
| hsa-miR-302a_st | 1.818872667 | 2.461408658 | 2.249238648 | 2.421394794 | 3.117085479 | 1.15466028 | 0.760130488 |
| hsa-miR-219-2-3p_st | 6.899954429 | 6.438641496 | 6.790768836 | 6.670673502 | 7.616638071 | 7.105163476 | 6.500456716 |
| hsa-miR-34b-star_st | 3.771201434 | 5.105646879 | 6.703511355 | 6.63403825 | 3.97003028 | 3.777145167 | 4.86408088 |
| hsa-miR-34b_st | 6.775058195 | 5.908339554 | 6.267935353 | 4.886271339 | 7.450826459 | 6.135950456 | 4.934693032 |
| hsa-miR-34c-5p_st | 2.569358485 | 2.482194489 | 3.05156833 | 2.185736736 | 3.157294511 | 2.934435035 | 2.250833696 |
| hsa-miR-34c-3p_st | 6.223597126 | 5.3698315 | 5.649786395 | 5.780885713 | 6.303876534 | 6.835290271 | 6.334604844 |
| hsa-miR-299-5p_st | 4.950422237 | 4.678760546 | 4.522162223 | 4.602791037 | 5.5426922 | 6.30402748 | 6.446749465 |
| hsa-miR-299-3p_st | 6.173380829 | 6.162643491 | 6.835452736 | 8.074913195 | 7.040893178 | 7.389118274 | 6.849599568 |
| hsa-miR-301a_st | 2.748480666 | 4.239985163 | 6.177645414 | 5.237205457 | 4.406466449 | 2.825312441 | 4.463063692 |
| hsa-miR-99b_st | 10.13820818 | 11.16140547 | 11.79850838 | 11.94543609 | 10.11384671 | 10.3383226 | 11.30991958 |
| hsa-miR-99b-star_st | 8.105670646 | 7.597073764 | 7.785265162 | 8.03851155 | 7.910051702 | 8.083388458 | 7.129505076 |
| hsa-miR-296-5p_st | 4.372488258 | 4.191816415 | 3.952109545 | 3.280387923 | 4.989515121 | 4.655409834 | 3.770778919 |
| hsa-miR-296-3p_st | 11.51907546 | 11.23077833 | 10.92230689 | 11.32972499 | 11.32792531 | 11.98606173 | 11.68241726 |
| hsa-miR-130b-star_st | 4.191301883 | 3.895084828 | 4.28694144 | 4.395341261 | 4.540856081 | 4.089406352 | 3.040887664 |
| hsa-miR-130b_st | 7.956017022 | 7.650604762 | 9.389567266 | 8.502937452 | 6.972951972 | 7.929065823 | 7.565687386 |
| hsa-miR-30 [symbol] _st | 9.232191902 | 9.303211263 | 10.70646239 | 11.44577569 | 9.27682026 | 8.240636673 | 8.655339995 |
| hsa-miR-30 [symbol] -star_st | 8.494628404 | 8.371277672 | 9.551668823 | 10.64897779 | 8.737289833 | 6.198045921 | 7.936274296 |
| hsa-miR-26a-2-star_st | 0.244237035 | 0.764557411 | 1.044437823 | 3.495767246 | 0.327660441 | −0.228618088 | 1.072277438 |
| hsa-miR-361-5p_st | 10.58812464 | 10.85021483 | 11.73611213 | 11.4796971 | 9.765335862 | 10.1677554 | 10.36498951 |
| hsa-miR-361-3p_st | 5.747118185 | 4.881073277 | 6.285243737 | 7.235577328 | 5.750719479 | 5.880051395 | 4.964220924 |
| hsa-miR-362-5p_st | 6.593791258 | 6.42866353 | 7.579314487 | 7.254003573 | 6.302470499 | 7.014645838 | 5.810450013 |
| hsa-miR-362-3p_st | 2.712235861 | 3.310294674 | 4.371779538 | 5.974499216 | 3.433370912 | 2.632662563 | 1.961924908 |
| hsa-miR-363-star_st | 10.47213715 | 9.763029519 | 9.306418504 | 9.455709928 | 9.924571943 | 9.856413018 | 10.31402515 |
| hsa-miR-363_st | 5.728431766 | 4.511752982 | 7.149971078 | 6.294731199 | 5.215647861 | 4.012664934 | 4.300000954 |
| hsa-miR-365_st | 3.522290291 | 2.669814289 | 3.750861188 | 3.89427793 | 2.580516873 | 1.507411815 | 2.40054963 |
| hsa-miR-365-star_st | 10.75938149 | 10.38724188 | 10.22970146 | 10.55777404 | 10.71054415 | 11.5138193 | 11.36758467 |
| hsa-miR-302b-star_st | 3.436658334 | 4.038382435 | 3.205584347 | 3.619448918 | 2.701445033 | 2.998349206 | 3.358653139 |
| hsa-miR-302b_st | −0.423071411 | 1.297733625 | 0.450001229 | 1.12165908 | 0.194614368 | −0.836145201 | 0.340801348 |
| hsa-miR-302c-star_st | 7.119359015 | 7.239686523 | 7.160367409 | 6.67915066 | 7.244134197 | 7.917265947 | 7.476687232 |
| hsa-miR-302c_st | 1.375021698 | 1.939934228 | 1.913785856 | 3.12364524 | 1.780628403 | 0.377522923 | 1.612148806 |
| hsa-miR-302d-star_st | 6.786884487 | 6.467950757 | 6.232364399 | 5.834539825 | 7.741466955 | 6.229415235 | 6.171797895 |
| hsa-miR-302d_st | 0.931091422 | 1.465251896 | 1.617855999 | 0.818258323 | 1.893609301 | 0.148932348 | 0.949523386 |
| hsa-miR-367-star_st | 1.267906574 | 1.410447263 | 0.317229568 | −0.252566081 | 1.16283862 | 1.053480827 | 0.718642506 |
| hsa-miR-367_st | 1.286000411 | 1.814754325 | 1.490102954 | 1.826484384 | 0.116571705 | 0.803147056 | 1.489776233 |
| hsa-miR-376c_st | 2.850707327 | 3.852250516 | 4.454235572 | 4.762576969 | 3.66030209 | 5.983911455 | 7.022262395 |
| hsa-miR-369-5p_st | 2.816605982 | 3.429502238 | 2.967445772 | 2.041933545 | 3.902130115 | 4.573546486 | 5.92064661 |
| hsa-miR-369-3p_st | 0.584522362 | 0.527524997 | 0.435511191 | −0.21088085 | 1.555950965 | 0.838843072 | 0.149038903 |
| hsa-miR-370_st | 10.66250198 | 10.19542595 | 9.934866716 | 10.41134399 | 10.49497141 | 11.66197987 | 11.15441774 |
| hsa-miR-371-5p_st | 11.46805808 | 10.65296546 | 10.84385006 | 11.0967639 | 11.3071377 | 12.40076071 | 11.59256738 |
| hsa-miR-371-3p_st | 4.428684445 | 4.00951213 | 2.927362451 | 3.780362405 | 4.951871521 | 3.672238769 | 3.593820418 |
| hsa-miR-372_st | 3.383842566 | 2.932912173 | 2.695467555 | 2.868907903 | 2.911392346 | 3.410624225 | 2.083169638 |
| hsa-miR-373-star_st | 10.44198357 | 9.834390326 | 9.660602823 | 10.18923427 | 10.24367291 | 11.50611211 | 10.8012473 |
| hsa-miR-373_st | 4.68157357 | 4.667159466 | 5.347240899 | 4.898623484 | 5.760515338 | 4.188565291 | 3.565890765 |
| hsa-miR-374a_st | 2.746352732 | 2.101453857 | 4.790475604 | 4.987806416 | 3.68464203 | 1.55251603 | 2.308989051 |
| hsa-miR-374a-star_st | 2.359595217 | 2.707385191 | 3.277990385 | 2.993706106 | 1.462605794 | 1.989512609 | 2.472470429 |
| hsa-miR-375_st | 3.686513562 | 6.426353038 | 6.137216321 | 8.152571828 | 4.938681056 | 5.561218031 | 11.10838014 |
| hsa-miR-376a-star_st | 0.245067577 | 0.71757059 | 0.377714058 | 1.031179431 | 0.394770958 | −0.486256129 | 0.297106535 |
| hsa-miR-376a_st | 3.241543741 | 4.201065686 | 3.424938745 | 3.19523485 | 3.538782724 | 4.01732044 | 5.004864937 |
| hsa-miR-377-star_st | 3.761491855 | 3.885023279 | 4.217497117 | 4.151183189 | 3.90914576 | 4.090239155 | 5.01639604 |
| hsa-miR-377_st | 2.017976063 | 2.501149813 | 2.461234646 | 2.99185006 | 2.074998783 | 2.653150198 | 3.238371152 |
| hsa-miR-378-star_st | 4.936781413 | 5.157465089 | 5.063804703 | 4.719172179 | 5.30446115 | 5.129536145 | 5.168817953 |
| hsa-miR-378_st | 8.67122022 | 7.99057416 | 8.609338708 | 8.587733241 | 8.907842308 | 9.040023431 | 8.808561769 |
| hsa-miR-379_st | 5.056938067 | 4.739756204 | 5.49523466 | 5.597989582 | 6.258519917 | 5.211109664 | 7.062890441 |
| hsa-miR-379-star_st | 2.332267711 | 2.481254736 | 1.336725792 | 2.563032243 | 0.97963515 | 1.601561422 | 3.006174657 |
| hsa-miR-380-star_st | 2.500714412 | 1.249601515 | 2.216234747 | 1.44333619 | 2.22407685 | 1.298198913 | 2.368648228 |
| hsa-miR-380_st | 1.522644209 | 0.714126368 | −0.175728294 | 0.564272055 | −0.332641745 | 0.974600959 | 1.349426779 |
| hsa-miR-381_st | 5.195650439 | 5.258783721 | 4.329945176 | 4.302408997 | 6.312257649 | 5.895777269 | 5.871400479 |
| hsa-miR-382_st | 6.376664164 | 7.003910777 | 5.929397284 | 6.085575512 | 6.076890881 | 6.521354915 | 8.654419679 |
| hsa-miR-383_st | 6.150586869 | 5.482646286 | 5.883540209 | 5.206614207 | 6.797733986 | 5.654344534 | 4.991290529 |
| hsa-miR-340_st | 2.739748114 | 4.226724273 | 4.888614384 | 5.626948873 | 4.294456897 | 3.151236965 | 3.7316651 |
| hsa-miR-340-star_st | 1.105858998 | 1.868313575 | 1.909870054 | 1.003833926 | 2.432430935 | 0.75420138 | 1.390787536 |
| hsa-miR-330-5p_st | 5.543035505 | 6.00995518 | 5.169021449 | 5.431741752 | 6.380199848 | 6.063818017 | 6.123791792 |
| hsa-miR-330-3p_st | 5.877504201 | 5.899891653 | 7.128009078 | 6.079233466 | 5.952005285 | 6.094368129 | 6.604788802 |
| hsa-miR-328_st | 8.023271414 | 6.94536591 | 6.754418019 | 7.908697225 | 8.042126529 | 7.70300363 | 7.701877459 |
| hsa-miR-342-5p_st | 6.013896834 | 5.130002933 | 5.635206131 | 5.654050909 | 3.250929013 | 4.970118357 | 4.892404436 |
| hsa-miR-342-3p_st | 11.18782558 | 10.33383011 | 10.55575686 | 11.04744498 | 9.716525489 | 9.987005921 | 10.72063988 |
| hsa-miR-337-5p_st | 3.11616758 | 2.495193726 | 2.529643134 | 2.711052765 | 2.407500661 | 2.184134061 | 4.933427389 |
| hsa-miR-337-3p_st | 4.192662148 | 4.163497455 | 4.241856453 | 3.338704997 | 4.729829498 | 3.458832889 | 3.928510968 |
| hsa-miR-323-5p_st | 10.68533297 | 10.43832356 | 9.988308198 | 10.55372502 | 10.39694144 | 10.83355004 | 11.2476582 |
| hsa-miR-323-3p_st | 3.172662581 | 3.917278932 | 3.518027496 | 4.338639515 | 2.871411534 | 3.898537407 | 5.079618197 |
| hsa-miR-326_st | 7.189387683 | 6.434684307 | 6.521292472 | 6.97468923 | 6.62066558 | 6.132429218 | 6.844870951 |
| hsa-miR-151-5p_st | 10.84197149 | 11.23883523 | 12.76668197 | 12.60927027 | 9.914487993 | 10.4084821 | 11.19280892 |
| hsa-miR-151-3p_st | 8.067053149 | 8.23323805 | 9.960454629 | 9.933906889 | 6.58645934 | 8.707401804 | 8.466364616 |
| hsa-miR-135b_st | 0.920062746 | 1.906618649 | 3.15237334 | 3.467772674 | 2.708207518 | 0.611852554 | 1.322383227 |
| hsa-miR-135b-star_st | 5.779762829 | 6.185345105 | 5.901408556 | 6.116182373 | 6.817866651 | 6.725849204 | 6.054944542 |
| hsa-miR-148b-star_st | 2.113008009 | 2.469794658 | 2.480660288 | 3.105552475 | 2.645119452 | 1.371280818 | 1.263135905 |
| hsa-miR-148b_st | 7.552957328 | 8.728074879 | 9.789143638 | 10.61861591 | 6.207270443 | 6.554188772 | 8.7554851 |
| hsa-miR-331-5p_st | 5.84637556 | 6.058363224 | 6.880749831 | 6.007233777 | 6.287541051 | 6.479721079 | 5.885213925 |
| hsa-miR-331-3p_st | 8.43599499 | 8.871346705 | 10.78920451 | 10.73465829 | 8.121097469 | 7.973937898 | 9.827228306 |
| hsa-miR-324-5p_st | 7.657052602 | 8.452394225 | 10.09490014 | 9.7779192 | 7.348160894 | 7.899566703 | 9.009269421 |
| hsa-miR-324-3p_st | 7.826858127 | 7.999003888 | 9.107038126 | 8.270187988 | 8.282632625 | 8.008809916 | 7.74731467 |
| hsa-miR-338-5p_st | 4.561652104 | 5.187278435 | 6.573332418 | 6.152560126 | 5.237665476 | 5.282553326 | 4.830155015 |
| hsa-miR-338-3p_st | 3.374302226 | 3.761520049 | 6.700619906 | 6.527609825 | 4.508076527 | 2.889486257 | 3.9678053 |
| hsa-miR-339-5p_st | 7.969051484 | 7.796328417 | 8.765662152 | 8.200420366 | 7.351144211 | 8.086352477 | 7.718630707 |
| hsa-miR-339-3p_st | 8.3692502 | 8.607951369 | 8.899536514 | 8.764571056 | 8.068342249 | 8.436200024 | 7.943722012 |
| hsa-miR-335_st | 7.671713581 | 8.287737938 | 10.39543188 | 9.848927583 | 7.228803961 | 6.102533735 | 10.15294873 |
| hsa-miR-335-star_st | 4.082026326 | 2.361811507 | 3.225032342 | 3.793966342 | 4.624678977 | 1.814977209 | 2.87063635 |
| hsa-miR-133b_st | 4.414634066 | 5.210705263 | 5.682681058 | 5.547738416 | 4.21831562 | 3.997423643 | 5.298707856 |
| hsa-miR-325_st | 3.853904368 | 3.257803028 | 3.604437494 | 3.418319871 | 3.743732192 | 3.255085399 | 4.244909852 |
| hsa-miR-345_st | 9.373440995 | 9.480434043 | 12.28027146 | 11.41637476 | 9.079104101 | 8.409632131 | 5.980464413 |
| hsa-miR-346_st | 8.784388859 | 8.167658397 | 8.205981074 | 8.158354848 | 8.900148273 | 8.967807936 | 9.339839674 |
| hsa-miR-384_st | 0.842931018 | 1.258444499 | 0.95981369 | 0.678065129 | −0.147713731 | 0.155753324 | 0.479215017 |
| hsa-miR-196b_st | 1.76546443 | 2.531502363 | 3.205204927 | 2.805552739 | 2.728638755 | 1.928151386 | 1.71009496 |
| hsa-miR-196b-star_st | 5.779327792 | 6.178843645 | 5.654104379 | 5.792737568 | 6.210381189 | 6.491260119 | 6.246087963 |
| hsa-miR-422a_st | 8.459954474 | 8.377322163 | 8.533880591 | 9.237399557 | 9.619792287 | 9.062528905 | 9.037294785 |
| hsa-miR-423-5p_st | 12.89182799 | 12.62140973 | 12.4055162 | 12.54246929 | 12.86180596 | 13.35382633 | 13.138973 |
| hsa-miR-423-3p_st | 9.063235932 | 9.185441861 | 10.64425982 | 9.719892765 | 8.924912467 | 8.963259279 | 9.506448899 |
| hsa-miR-424_st | 4.882868691 | 6.295135013 | 7.994906891 | 10.84215501 | 5.46745459 | 5.834776276 | 3.70676654 |
| hsa-miR-424-star_st | 8.029179231 | 8.043883588 | 8.337879539 | 9.271244311 | 7.478962877 | 8.29953306 | 7.305267459 |
| hsa-miR-425_st | 8.582498398 | 8.352634808 | 10.30500075 | 9.41102455 | 8.140395514 | 9.341932874 | 8.396795161 |
| hsa-miR-425-star_st | 8.098387011 | 7.932382183 | 7.829302142 | 7.532967708 | 7.438925211 | 8.533314119 | 8.000824764 |
| hsa-miR-18b_st | 4.499532941 | 3.728748147 | 4.786809911 | 4.110511493 | 4.186012317 | 3.601074213 | 2.735250315 |
| hsa-miR-18b-star_st | 4.287819139 | 4.077068701 | 4.43673482 | 4.765473323 | 4.963154947 | 4.650173468 | 4.400712134 |
| hsa-miR-20b_st | 7.865800708 | 8.202315551 | 9.532464877 | 9.788243423 | 7.569925978 | 7.566182684 | 6.843484444 |
| hsa-miR-20b-star_st | 4.143627157 | 5.030917652 | 4.206523494 | 5.175876296 | 5.05688342 | 4.974647314 | 4.459519972 |
| hsa-miR-448_st | 2.162557404 | 2.214686596 | 2.13922308 | 2.774330799 | 2.538956688 | 2.210529503 | 1.720363848 |
| hsa-miR-429_st | 4.241260167 | 6.081711471 | 5.577460139 | 7.480276828 | 4.434648205 | 3.527725747 | 5.935416594 |
| hsa-miR-449a_st | 3.045092947 | 3.665909815 | 3.456066367 | 3.1817055 | 3.711956766 | 2.698347049 | 2.48748893 |
| hsa-miR-450a_st | 1.536042121 | 3.616575704 | 4.144006502 | 5.168090675 | 2.83111974 | 1.897887798 | 2.184613142 |
| hsa-miR-431_st | 2.915260253 | 2.755098244 | 3.288321387 | 2.74860232 | 2.483698739 | 3.580672381 | 2.944661195 |
| hsa-miR-431-star_st | 5.505880555 | 6.038798865 | 5.880931061 | 6.244159874 | 6.812791054 | 6.629710406 | 7.124071842 |
| hsa-miR-433_st | 5.517735236 | 4.554620367 | 4.395937844 | 4.312870005 | 4.893548608 | 5.397555859 | 6.951844216 |
| hsa-miR-329_st | 3.398369888 | 3.510061867 | 3.285220855 | 2.868015751 | 3.671430905 | 3.772272093 | 6.797868256 |
| hsa-miR-451_st | 12.96940173 | 11.82769992 | 13.0955659 | 14.19532705 | 9.950243634 | 11.81095118 | 10.98415674 |
| hsa-miR-452_st | 5.710855215 | 5.595672484 | 7.330620363 | 7.74641091 | 7.445811206 | 6.721417294 | 6.17210208 |
| hsa-miR-452-star_st | 3.528180907 | 3.615676975 | 2.71971657 | 3.132175727 | 2.842837796 | 2.856532589 | 3.529203911 |
| hsa-miR-409-5p_st | 3.374220979 | 3.169664112 | 3.45247325 | 3.947970815 | 3.349906107 | 3.50555313 | 5.245901773 |
| hsa-miR-409-3p_st | 5.729595433 | 5.453170437 | 4.543197836 | 5.391541448 | 6.291002505 | 7.390994388 | 7.780689088 |
| hsa-miR-412_st | 3.853293934 | 4.254169686 | 4.474206596 | 4.170368141 | 4.989744029 | 4.541766664 | 3.938177965 |
| hsa-miR-410_st | 3.72222715 | 3.670557803 | 3.258032713 | 2.91539165 | 2.903733938 | 3.484047063 | 5.408722177 |
| hsa-miR-376b_st | 2.048566137 | 2.729773534 | 1.641543446 | 2.68496279 | 1.437631545 | 2.482683177 | 3.602866796 |
| hsa-miR-483-5p_st | 10.9378543 | 10.8656964 | 10.71265813 | 11.03548838 | 11.09259711 | 11.58218504 | 11.45556196 |
| hsa-miR-483-3p_st | 6.665855363 | 6.316505497 | 5.951720747 | 6.316170882 | 6.850657716 | 7.20191699 | 7.033686048 |
| hsa-miR-484_st | 6.913860167 | 6.786655549 | 8.619578993 | 7.127768873 | 6.55347334 | 7.193164653 | 7.974155786 |
| hsa-miR-485-5p_st | 5.94920657 | 5.676783967 | 5.581727432 | 5.613559599 | 6.384470949 | 6.322337878 | 7.407799826 |
| hsa-miR-485-3p_st | 5.291253144 | 4.907830691 | 5.659977304 | 4.972062969 | 5.999860274 | 6.003767517 | 5.690373839 |
| hsa-miR-486-5p_st | 8.69158319 | 7.174199322 | 7.760496764 | 8.886961688 | 7.276771701 | 7.586422584 | 8.491068116 |
| hsa-miR-486-3p_st | 9.954956254 | 9.010921276 | 8.727808912 | 9.299464961 | 9.553294049 | 10.11271732 | 9.965941934 |
| hsa-miR-487a_st | 5.623884505 | 4.975835559 | 4.93582479 | 4.916795819 | 5.852231815 | 5.57826735 | 5.763071109 |
| hsa-miR-488-star_st | 2.500680883 | 2.381066254 | 1.973024867 | 2.332466917 | 1.895005876 | 1.321182229 | 2.035841781 |
| hsa-miR-488_st | 2.62719596 | 2.316401956 | 2.348805102 | 2.271105315 | 1.122737217 | 1.885344241 | 1.725288171 |
| hsa-miR-489_st | 7.508636931 | 7.282778511 | 7.967696477 | 8.419882862 | 9.129564931 | 8.874439074 | 9.15128658 |
| hsa-miR-490-5p_st | 6.63468389 | 6.029808769 | 6.071069604 | 5.980226978 | 6.734694937 | 7.554556125 | 7.686641955 |
| hsa-miR-490-3p_st | 5.082500625 | 4.652859918 | 4.195431463 | 4.747161999 | 5.2866652 | 5.649061601 | 5.248693968 |
| hsa-miR-491-5p_st | 9.132643854 | 8.112016789 | 9.536157287 | 8.948854356 | 8.598623181 | 8.952183645 | 9.462878083 |
| hsa-miR-491-3p_st | 5.54326012 | 4.65990261 | 4.932498107 | 4.469863838 | 5.398481819 | 4.864612776 | 4.335984162 |
| hsa-miR-511_st | 0.839436228 | 0.113024582 | 2.244800081 | 0.282334695 | 1.693281708 | −0.347514889 | 0.83389021 |
| hsa-miR-146b-5p_st | 10.52057441 | 14.70966445 | 12.5161599 | 14.32373111 | 8.454725246 | 13.37425573 | 8.075023822 |
| hsa-miR-146b-3p_st | 4.719539775 | 7.799741716 | 5.880215126 | 7.941664517 | 3.481937957 | 6.03691075 | 2.720996282 |
| hsa-miR-202-star_st | −0.710354353 | 0.11688602 | 0.110264034 | −0.058861623 | −0.086572407 | 0.429165649 | −1.034188406 |
| hsa-miR-202_st | 8.374365459 | 8.118762885 | 8.061756989 | 7.908788593 | 8.604166618 | 8.405821408 | 8.630164513 |
| hsa-miR-492_st | 7.629427245 | 7.426256936 | 7.163710438 | 8.22053489 | 8.105177913 | 9.051248437 | 8.499085675 |
| hsa-miR-493-star_st | 3.487163308 | 4.345678887 | 4.011155197 | 3.855518329 | 4.298424359 | 4.712630615 | 4.227656678 |
| hsa-miR-493_st | 5.385862991 | 4.795534507 | 5.101901016 | 4.735788463 | 4.868820869 | 5.310596275 | 4.890299928 |
| hsa-miR-432_st | 6.54502925 | 6.003396028 | 6.412921209 | 6.410218113 | 7.38860191 | 6.965490916 | 8.774912478 |
| hsa-miR-432-star_st | 4.596185241 | 4.302474656 | 4.444028932 | 4.320177292 | 5.384393998 | 5.244918718 | 5.33587688 |
| hsa-miR-494_st | 13.98461275 | 14.88118304 | 14.42031419 | 15.12039241 | 14.52981296 | 15.6143693 | 15.53535125 |
| hsa-miR-495_st | 2.262903045 | 2.734790533 | 2.143386819 | 3.889029154 | 2.476828061 | 3.530585983 | 6.876033135 |
| hsa-miR-496_st | 0.472655191 | 1.643274745 | 0.299584845 | 1.603247092 | 1.643717548 | 1.359192885 | 2.288260374 |
| hsa-miR-193b-star_st | 11.01101258 | 10.62378697 | 10.26195889 | 10.47300255 | 10.6684967 | 11.54872907 | 10.94864216 |
| hsa-miR-193b_st | 8.435169833 | 8.163186009 | 8.985803483 | 9.670567235 | 6.18975779 | 8.675079249 | 7.392970829 |
| hsa-miR-497_st | 9.050640456 | 8.244639698 | 10.66736358 | 10.29175183 | 8.484254593 | 7.996287595 | 9.293549196 |
| hsa-miR-497-star_st | 1.643786965 | 2.047643771 | 0.979924947 | 1.876203663 | 1.496653186 | 2.117945865 | 2.494677383 |
| hsa-miR-181d_st | 8.420723783 | 9.41676115 | 9.827333839 | 11.43832275 | 8.048719709 | 8.857936017 | 8.506563185 |
| hsa-miR-512-5p_st | 4.586588427 | 4.291934453 | 3.468613759 | 4.596197901 | 5.00137836 | 4.196364425 | 3.957253756 |
| hsa-miR-512-3p_st | 4.395390302 | 4.35000214 | 4.216604755 | 3.456111122 | 4.385710511 | 4.211822087 | 4.219703533 |
| hsa-miR-498_st | 11.09428756 | 10.95435299 | 9.8331356 | 10.41129628 | 10.19214144 | 11.81207563 | 11.35393276 |
| hsa-miR-520 [symbol] _st | 1.675954968 | 2.119538988 | 2.120835759 | 1.209729103 | 1.191246989 | 1.463702792 | 0.755242281 |
| hsa-miR-515-5p_st | 1.662186444 | 2.257666226 | 2.462623701 | 2.024098196 | 2.062043214 | 2.778166658 | 2.595360763 |
| hsa-miR-515-3p_st | 4.375396132 | 3.808048853 | 3.39352862 | 3.182907393 | 5.106154805 | 2.428022429 | 3.275411466 |
| hsa-miR-519 [symbol] -star_st | 5.729696842 | 5.097101852 | 5.204551906 | 4.550616919 | 6.608884358 | 6.767449272 | 5.985567073 |
| hsa-miR-519 [symbol] _st | 3.471908391 | 3.842707686 | 4.099387073 | 3.335056747 | 3.60342135 | 3.582457983 | 3.89475721 |
| hsa-miR-520 [symbol] _st | 1.685083081 | 1.394834323 | 2.703273015 | 2.863401672 | 1.713775331 | 0.542016529 | 1.530500218 |
| hsa-miR-519c-5p_st | 5.406237378 | 4.186498999 | 5.110205282 | 4.490789316 | 5.670155936 | 5.747092568 | 5.910440404 |
| hsa-miR-519c-3p_st | −0.549794826 | 0.622617686 | −0.33511625 | −1.317412983 | −0.483598918 | −0.50596838 | −0.136204987 |
| hsa-miR-520a-5p_st | 4.519500171 | 3.667123568 | 2.500904717 | 2.357004003 | 4.112836347 | 3.904275982 | 3.207414766 |
| hsa-miR-520a-3p_st | 3.10388247 | 2.000657387 | 2.901119578 | 2.846109152 | 3.679453058 | 1.691957609 | 1.843885913 |
| hsa-miR-526b_st | 4.949959327 | 4.459591212 | 4.674527623 | 4.444358449 | 5.686353998 | 4.431356481 | 4.420912356 |
| hsa-miR-526b-star_st | 0.60233174 | 1.155060732 | 2.186238509 | −0.152210402 | 1.382461222 | 1.444201582 | 0.837489073 |
| hsa-miR-519b-5p_st | 5.764440067 | 5.153931951 | 5.253103802 | 6.056261066 | 6.217745987 | 5.721172117 | 5.571879494 |
| hsa-miR-519b-3p_st | 1.725319737 | 1.890360411 | 1.780752816 | 0.733844217 | 0.878526856 | 0.874462175 | 0.55147417 |
| hsa-miR-525-5p_st | 4.914893377 | 4.939616585 | 4.525264862 | 4.38321538 | 5.197370701 | 5.171392936 | 5.539146207 |
| hsa-miR-525-3p_st | 3.136624386 | 2.908171583 | 2.228155508 | 2.108242343 | 1.698417783 | 3.27746518 | 2.720377479 |
| hsa-miR-523-star_st | 5.641050698 | 3.878241586 | 3.859254339 | 5.070155783 | 5.42496753 | 5.707575993 | 5.032216476 |
| hsa-miR-523_st | 1.033488418 | 1.586070682 | 1.324662699 | 1.103658375 | 0.385425412 | 1.439021362 | 1.12957409 |
| hsa-miR-518 [symbol] -star_st | 6.482487786 | 5.833422436 | 5.545088839 | 5.644723701 | 5.96857591 | 6.279083636 | 6.454918966 |
| hsa-miR-518 [symbol] _st | 0.998159165 | 1.148713076 | 1.56831707 | 0.694297378 | 0.702306121 | 1.314675628 | 0.632210105 |
| hsa-miR-520b_st | 1.03456029 | 1.217354164 | 1.043293188 | −0.03949637 | 0.515829828 | 1.108856635 | 1.138783346 |
| hsa-miR-518b_st | 3.438524426 | 2.717213079 | 3.075565546 | 3.049839255 | 2.920466083 | 3.565723937 | 4.380235243 |
| hsa-miR-526a_st | 6.032071983 | 5.072921146 | 5.879985174 | 5.67980207 | 6.689122741 | 5.989887504 | 5.963522371 |
| hsa-miR-520c-5p_st | 5.765271582 | 5.693138417 | 6.13562055 | 6.552868785 | 6.401170506 | 6.345762923 | 6.17289623 |
| hsa-miR-520c-3p_st | 2.247390942 | 3.446986383 | 3.226686882 | 3.629892063 | 3.379888391 | 2.997761269 | 2.476401929 |
| hsa-miR-518c-star_st | 8.147354578 | 7.078271483 | 7.932173465 | 7.615708413 | 8.45859527 | 8.381858995 | 8.396162316 |
| hsa-miR-518c_st | −0.777517245 | 0.6041063 | −0.526736849 | −0.231989003 | 0.518111246 | 0.119043374 | 0.086523521 |
| hsa-miR-524-5p_st | 5.459834811 | 4.504010275 | 5.193528956 | 4.932016145 | 5.194604662 | 4.262254725 | 4.347500832 |
| hsa-miR-524-3p_st | 2.735798049 | 2.915233161 | 2.781078582 | 1.836713128 | 2.866370074 | 2.651850418 | 3.326497246 |
| hsa-miR-517-star_st | 4.761400007 | 4.419790058 | 4.250741056 | 4.072639789 | 4.580273113 | 4.822990606 | 4.368774133 |
| hsa-miR-517a_st | 2.195131565 | 1.210558881 | 0.359337297 | 1.73778594 | 2.210479709 | 0.589857448 | 1.842891979 |
| hsa-miR-519d_st | 4.132793585 | 3.626304786 | 3.693808848 | 3.780643324 | 3.861885802 | 3.044223817 | 3.814302266 |
| hsa-miR-521_st | 2.489355502 | 2.722874793 | 3.086840786 | 3.554839091 | 3.123897523 | 2.738635801 | 2.730466676 |
| hsa-miR-520d-5p_st | 6.195598862 | 4.882337586 | 5.846500299 | 5.893878744 | 6.194230037 | 6.616149745 | 7.007784209 |
| hsa-miR-520d-3p_st | 5.323296403 | 5.699770398 | 5.191000218 | 4.372326295 | 6.419796604 | 5.469519667 | 3.952336282 |
| hsa-miR-517b_st | 2.760911435 | 2.926054466 | 3.233010063 | 2.55525226 | 3.364069447 | 2.291932494 | 2.580645489 |
| hsa-miR-520g_st | 2.369255034 | 1.914940511 | 2.616487955 | 1.525505264 | 3.180571513 | 1.433766715 | 1.873566369 |
| hsa-miR-516b_st | 5.882338411 | 5.510166251 | 5.857692753 | 6.502369011 | 6.70047697 | 6.514763305 | 7.211128145 |
| hsa-miR-516b-star_st | 2.801934818 | 3.321359042 | 2.660297327 | 3.63093769 | 2.177969079 | 2.791774524 | 3.144125074 |
| hsa-miR-518 [symbol] -star_st | 6.199327696 | 5.56484411 | 5.939852567 | 5.447737281 | 6.28953341 | 6.017023596 | 5.708195652 |
| hsa-miR-518 [symbol] _st | 2.469889705 | 1.625959269 | 2.524813956 | 0.298813904 | 1.160099192 | 1.442571747 | 1.322047396 |
| hsa-miR-518a-5p_st | 6.4065813 | 6.03862878 | 5.852241749 | 7.068438045 | 6.782664753 | 6.790318254 | 6.642328151 |
| hsa-miR-518a-3p_st | 3.009493472 | 3.049731066 | 2.797335983 | 1.918310434 | 3.685969858 | 2.902829381 | 2.950615287 |
| hsa-miR-518d-5p_st | 6.005551575 | 5.986588652 | 5.548331989 | 5.737001891 | 6.020136865 | 6.187263916 | 6.122531729 |
| hsa-miR-518d-3p_st | 3.033963685 | 2.856131336 | 2.311075845 | 2.069450896 | 2.771209718 | 3.207793422 | 2.597781453 |
| hsa-miR-517c_st | −0.189209395 | −0.123112626 | 0.864703373 | −0.568531577 | 0.665877394 | −0.396534366 | −0.543525556 |
| hsa-miR-520h_st | 3.501857005 | 2.322431935 | 1.558945645 | 1.509406498 | 2.729457706 | 1.159661952 | 2.364359378 |
| hsa-miR-522-star_st | 4.80791035 | 4.896525074 | 4.77136396 | 4.656568434 | 5.954806199 | 6.350870405 | 5.561035081 |
| hsa-miR-522_st | 2.226131081 | 2.337903259 | 1.531606335 | 1.357960144 | 1.617437867 | 1.846396545 | 1.931253577 |
| hsa-miR-519a-star_st | 5.582550105 | 4.354351578 | 3.686316205 | 4.313327802 | 5.884078752 | 5.867585927 | 5.287321982 |
| hsa-miR-519a_st | 0.248203506 | −0.147905597 | −0.525791863 | −1.516950308 | 1.318269063 | −0.341668399 | 0.22974033 |
| hsa-miR-527_st | 6.510959215 | 6.539492409 | 6.425150205 | 7.05391517 | 7.582825801 | 7.474668696 | 6.528886973 |
| hsa-miR-516a-5p_st | 7.088239464 | 5.918051974 | 6.10113407 | 6.304903174 | 7.645689315 | 7.502644792 | 6.969722152 |
| hsa-miR-516a-3p_st | 2.526175394 | 2.928041413 | 2.574533178 | 2.197902249 | 2.362803412 | 2.46902788 | 2.226040698 |
| hsa-miR-499-5p_st | 1.735921828 | 2.044475607 | 5.017299949 | 1.883292365 | 3.588723052 | 1.179388971 | 1.852773681 |
| hsa-miR-499-3p_st | 2.695462592 | 2.412555913 | 3.829239174 | 2.917713351 | 1.720955791 | 2.655627135 | 3.031909236 |
| hsa-miR-500_st | 6.115427242 | 5.62835908 | 7.429244352 | 6.412291828 | 5.313361614 | 6.964763976 | 5.313563745 |
| hsa-miR-500-star_st | 8.614855484 | 7.618592458 | 8.617485619 | 9.015593792 | 7.92392514 | 8.735283037 | 8.262721627 |
| hsa-miR-501-5p_st | 5.417892087 | 5.215368468 | 5.108110084 | 6.672488427 | 5.347400481 | 5.125839848 | 5.705047891 |
| hsa-miR-501-3p_st | 8.686883016 | 8.160224812 | 8.557588768 | 8.134681511 | 8.360146435 | 8.988086251 | 8.472333358 |
| hsa-miR-502-5p_st | 3.656195807 | 3.565752032 | 3.987136015 | 3.99529824 | 4.033136498 | 3.450045182 | 2.682136412 |
| hsa-miR-502-3p_st | 8.568606026 | 8.055237183 | 8.655385039 | 8.869641429 | 7.855702505 | 8.532227184 | 8.40085584 |
| hsa-miR-503_st | 10.05526134 | 10.1706024 | 9.413847157 | 10.1360777 | 9.361401731 | 10.41098089 | 10.5603285 |
| hsa-miR-504_st | 4.733945101 | 5.020624194 | 5.302854486 | 4.889820098 | 5.110689772 | 4.613444198 | 7.010663539 |
| hsa-miR-505-star_st | 7.70634597 | 6.47300211 | 6.994515549 | 6.20932306 | 7.609360342 | 6.799143364 | 7.799187604 |
| hsa-miR-505_st | 5.054478955 | 4.322556268 | 5.644079478 | 5.728498907 | 4.238066748 | 4.821662534 | 5.822139512 |
| hsa-miR-513a-5p_st | 12.01689483 | 11.58496937 | 12.52463938 | 12.55320373 | 12.72827673 | 12.986416 | 13.2956074 |
| hsa-miR-513a-3p_st | 1.016511924 | 1.141672097 | 0.129620969 | 1.693439417 | −0.285468892 | −0.146609393 | 2.030167931 |
| hsa-miR-506_st | 3.397460858 | 3.618035775 | 3.760978768 | 3.437165209 | 3.615280319 | 3.163944068 | 3.080310649 |
| hsa-miR-507_st | 1.100674178 | 1.004100805 | 0.60886341 | 0.54123601 | −0.642894488 | 0.987745931 | 0.580257436 |
| hsa-miR-508-5p_st | 7.397868422 | 7.461686451 | 6.932265908 | 7.766250837 | 7.686883188 | 8.354051807 | 8.153664691 |
| hsa-miR-508-3p_st | 0.737446251 | 0.240671264 | 2.195973871 | 1.210963063 | 1.074518478 | −0.942758103 | −0.4641357 |
| hsa-miR-509-5p_st | 4.746801979 | 3.960726433 | 3.935030653 | 4.749178874 | 4.726133269 | 4.953843274 | 5.122556162 |
| hsa-miR-509-3p_st | 4.930815364 | 4.859305139 | 4.546538826 | 4.819761932 | 4.753935273 | 4.684626424 | 5.22752142 |
| hsa-miR-510_st | 7.352735486 | 7.002023174 | 6.864634879 | 7.073059844 | 8.01525399 | 8.44943122 | 8.275843102 |
| hsa-miR-514_st | 0.673418293 | 1.457188618 | 1.502887324 | 1.147143084 | 1.026837954 | 0.142984671 | 0.30282737 |
| hsa-miR-532-5p_st | 7.689369219 | 6.938208079 | 8.761787862 | 7.959027824 | 7.019139979 | 7.918420758 | 7.816276508 |
| hsa-miR-532-3p_st | 7.816436888 | 7.294434331 | 8.361100734 | 7.505876724 | 7.521743758 | 7.10706053 | 7.084561314 |
| hsa-miR-455-5p_st | 4.521412053 | 3.257983972 | 4.76537493 | 3.478305384 | 3.323378506 | 2.024253674 | 3.536649142 |
| hsa-miR-455-3p_st | 8.206693177 | 7.514317435 | 8.653915268 | 8.15463075 | 5.913146909 | 8.224099002 | 8.369132895 |
| hsa-miR-539_st | 3.153650763 | 3.379348904 | 3.618465698 | 4.58923712 | 4.098023517 | 2.670317037 | 6.000677123 |
| hsa-miR-544_st | −0.141359602 | 1.111510233 | 0.083093059 | −0.383995203 | −0.735306156 | −0.368364077 | 0.098712429 |
| hsa-miR-545-star_st | 0.682048438 | 1.464484815 | 1.078180281 | 1.495054436 | 0.774006755 | 1.398538513 | 1.489820507 |
| hsa-miR-545_st | 0.868632223 | 1.208827741 | 0.679844962 | 1.257143574 | 0.456574965 | 0.613815493 | 0.466117673 |
| hsa-miR-487b_st | 6.996737933 | 7.38625027 | 6.745930614 | 7.792952177 | 7.444282099 | 7.604082287 | 9.649092395 |
| hsa-miR-551a_st | 3.946372902 | 3.778055745 | 3.909230297 | 3.791524174 | 4.910442287 | 3.943318951 | 4.238093192 |
| hsa-miR-552_st | 4.613782644 | 4.589706144 | 3.970053674 | 4.750346192 | 4.660621348 | 3.827499362 | 5.231974785 |
| hsa-miR-553_st | 1.735343108 | 1.772618625 | 2.864791347 | 2.363657481 | 3.361853896 | 2.145926338 | 1.317674988 |
| hsa-miR-554_st | 4.319162096 | 4.527996206 | 3.778726947 | 4.198853805 | 4.934639981 | 4.175954613 | 4.675567443 |
| hsa-miR-92b-star_st | 14.26896827 | 14.2551999 | 13.8539788 | 14.2035804 | 13.96380845 | 15.11340303 | 14.73220385 |
| hsa-miR-92b_st | 8.806231403 | 8.912214078 | 9.345529399 | 8.838861847 | 8.53259147 | 8.890814165 | 8.316847399 |
| hsa-miR-555_st | 3.623139219 | 3.154467464 | 3.83929359 | 3.489814694 | 4.036995081 | 3.397308061 | 4.035941073 |
| hsa-miR-556-5p_st | 0.954827423 | 1.10923269 | 2.015772652 | 2.016342979 | −0.004490902 | 0.278540747 | 0.666758045 |
| hsa-miR-556-3p_st | 0.420972567 | 2.347393955 | 1.137077879 | 1.877233372 | −0.058014804 | 0.656840155 | 1.003443138 |
| hsa-miR-557_st | 11.78996606 | 10.72289265 | 10.50920716 | 11.30501459 | 11.43225514 | 12.41063254 | 11.82504438 |
| hsa-miR-558_st | 1.23937357 | 1.824799535 | 1.871563193 | 1.539823914 | 0.891607833 | 0.905187829 | 1.814314038 |
| hsa-miR-559_st | 2.027442916 | 2.244306812 | 1.442942664 | 2.926935634 | 1.881057024 | 0.966171504 | 1.931322247 |
| hsa-miR-561_st | 0.525527386 | 1.398717571 | 0.044911966 | 0.488116646 | 0.064989008 | 1.744867685 | 1.374535555 |
| hsa-miR-562_st | 1.744592994 | 1.421181047 | 1.926107033 | 1.84997461 | 0.592225586 | 1.13183089 | 0.201682401 |
| hsa-miR-563_st | 2.823110138 | 2.818187645 | 2.058077479 | 0.574902877 | 2.772848127 | 2.841063867 | 2.901722703 |
| hsa-miR-564_st | 11.61571838 | 11.13188404 | 11.49093595 | 11.55048334 | 11.73302897 | 12.29933641 | 12.18463026 |
| hsa-miR-566_st | 7.421205274 | 6.875799367 | 7.121412317 | 6.832239534 | 6.796061261 | 7.116446456 | 6.96635027 |
| hsa-miR-567_st | 2.872484694 | 2.941223114 | 2.878808335 | 2.063521577 | 2.379389899 | 2.704682418 | 1.967826245 |
| hsa-miR-568_st | 0.841222709 | 1.003993292 | 0.268887928 | 0.408696941 | 0.320762432 | 1.370908157 | 0.180566245 |
| hsa-miR-551b-star_st | 10.144072 | 10.80207939 | 10.13827864 | 10.32452342 | 10.20140123 | 10.66845316 | 10.62981089 |
| hsa-miR-551b_st | 1.831658154 | 6.031653391 | 4.902501433 | 4.320087058 | 1.353296753 | 2.866569471 | 1.160939481 |
| hsa-miR-569_st | 2.059050402 | 2.197334883 | 2.111663405 | 2.605915412 | 0.954620449 | 1.822215514 | 2.393329094 |
| hsa-miR-570_st | 1.429336709 | 2.098681434 | 1.860837924 | 3.642386705 | 3.149490978 | 0.961394389 | 2.186083925 |
| hsa-miR-571_st | 3.380559295 | 3.932621755 | 4.05569966 | 4.219762216 | 4.02741426 | 4.051145852 | 3.674427609 |
| hsa-miR-572_st | 13.71328956 | 13.26232857 | 12.95563784 | 13.43772993 | 13.55392917 | 14.72647896 | 14.15597419 |
| hsa-miR-573_st | 0.915203499 | 0.538451784 | 1.314701061 | 0.487946924 | −0.025999063 | −0.327956891 | 0.369268137 |
| hsa-miR-574-5p_st | 8.174609455 | 7.129576405 | 7.638178162 | 6.590010225 | 7.816554564 | 7.683912518 | 7.239715927 |
| hsa-miR-574-3p_st | 9.354293239 | 9.366586673 | 9.811478169 | 9.79238808 | 8.68975847 | 9.323111322 | 9.696666588 |
| hsa-miR-575_st | 12.14134093 | 11.83164776 | 12.4880422 | 12.78663814 | 12.65936333 | 13.72040819 | 13.35082926 |
| hsa-miR-576-5p_st | 4.052735121 | 3.717853966 | 3.228303147 | 2.984571167 | 3.117416511 | 2.478943054 | 3.318799149 |
| hsa-miR-576-3p_st | 4.193011785 | 4.212058968 | 4.45060884 | 4.788974215 | 6.287818248 | 3.152824124 | 4.492082281 |
| hsa-miR-577_st | 0.024570477 | 1.039931121 | 0.770209806 | 0.689806029 | −0.214009666 | 1.615514663 | 0.678151308 |
| hsa-miR-578_st | 2.988410084 | 4.032868967 | 3.955262144 | 3.12961382 | 3.13101188 | 2.674756648 | 3.147223288 |
| hsa-miR-579_st | 1.418868431 | 2.376228639 | 2.248171119 | 2.256512879 | 2.174676686 | 2.220716733 | 1.68647074 |
| hsa-miR-580_st | 1.791515535 | 1.09459287 | 0.72258462 | −0.035363538 | 0.4917074 | 1.059918212 | 1.162670873 |
| hsa-miR-581_st | 0.7491545 | −0.475220183 | 0.096158816 | −1.386606475 | 0.387322965 | 0.618778518 | −0.439336625 |
| hsa-miR-582-5p_st | 1.780259411 | 1.546873342 | 2.157961771 | 1.695210767 | 1.683379377 | 0.128053684 | 2.114597632 |
| hsa-miR-582-3p_st | 2.42975747 | 3.132402344 | 2.797576005 | 2.04520976 | 2.461344034 | 3.23704303 | 2.089123902 |
| hsa-miR-583_st | 6.437128467 | 5.298685748 | 5.365335413 | 5.609664047 | 6.068005233 | 6.532552101 | 5.570598399 |
| hsa-miR-584_st | 7.080132522 | 6.971329144 | 7.555048719 | 6.922772245 | 7.842862351 | 7.477167691 | 7.12596509 |
| hsa-miR-585_st | 3.01292581 | 3.10636595 | 2.491330782 | 3.123460879 | 2.600657745 | 2.938880313 | 3.197363501 |
| hsa-miR-548a-3p_st | 0.318523022 | 1.19009688 | 1.994197598 | 0.385254853 | 1.381379122 | 0.094641151 | 0.528920974 |
| hsa-miR-586_st | 1.279270963 | 1.792583007 | 2.204418094 | 2.263989756 | 1.354538991 | 0.88987338 | 1.661892417 |
| hsa-miR-587_st | 2.118818492 | 2.894049312 | 2.697376727 | 2.978405153 | 3.329280525 | 2.455720163 | 2.807680688 |
| hsa-miR-548b-5p_st | 3.734335891 | 2.757118192 | 4.041867641 | 4.024962717 | 4.291748511 | 2.109944618 | 1.643056814 |
| hsa-miR-548b-3p_st | 1.642453437 | 2.774159234 | 3.117411899 | 2.622291887 | 3.193940661 | 1.955213834 | 1.153854152 |
| hsa-miR-588_st | 4.29062919 | 4.720786655 | 4.983628933 | 4.968883971 | 4.736342406 | 5.211103331 | 5.195347532 |
| hsa-miR-589_st | 3.887558236 | 3.883264401 | 3.023904246 | 3.037780882 | 4.25144117 | 3.611549864 | 3.27318682 |
| hsa-miR-589-star_st | 4.799672458 | 4.795739846 | 4.833408782 | 3.827913515 | 5.52340674 | 4.917918559 | 4.965984889 |
| hsa-miR-550_st | 11.16428246 | 10.40434353 | 10.95939665 | 11.16521616 | 11.59810924 | 11.66224552 | 11.90658708 |
| hsa-miR-550-star_st | 4.166682298 | 4.581326751 | 4.984841508 | 4.010560427 | 4.887346146 | 4.525312343 | 3.860527763 |
| hsa-miR-590-5p_st | 2.197286679 | 2.438730482 | 2.523408476 | 1.981748267 | 2.752292563 | 0.836372605 | 0.95864178 |
| hsa-miR-590-3p_st | 1.257587867 | 1.241829502 | −0.105779452 | 0.860934071 | 0.484016233 | 0.994981194 | 0.168746252 |
| hsa-miR-591_st | 2.783823616 | 2.474387368 | 2.909320393 | 1.791175076 | 2.866647577 | 1.706979281 | 1.287035249 |
| hsa-miR-592_st | 1.84097372 | 0.672747337 | 1.484629064 | 1.315995386 | 2.503190344 | 0.455950787 | 2.085752662 |
| hsa-miR-593-star_st | 9.332162645 | 8.114067792 | 8.21033438 | 7.65692913 | 9.410123896 | 8.79398727 | 8.9833194 |
| hsa-miR-593_st | 5.828127858 | 5.800627487 | 5.90187003 | 5.565114451 | 6.332444763 | 5.505037721 | 4.21496538 |
| hsa-miR-595_st | 7.351906685 | 6.790181727 | 6.997603857 | 7.031769941 | 7.128351546 | 7.282898507 | 7.177527435 |
| hsa-miR-596_st | 7.861566113 | 6.632444124 | 6.961116656 | 7.374355474 | 7.660386225 | 8.160538384 | 8.195744521 |
| hsa-miR-597_st | 2.436971759 | 1.581016067 | 1.677023261 | 1.461927199 | 0.905204105 | 1.772202005 | 1.099577698 |
| hsa-miR-598_st | 5.331619976 | 5.720871401 | 7.434135124 | 7.968328453 | 5.348142934 | 4.250945032 | 7.347296647 |
| hsa-miR-599_st | 1.586356324 | 1.564198749 | 2.09465854 | 2.389020175 | 1.651481111 | 1.004264288 | 1.097614472 |
| hsa-miR-548a-5p_st | 3.635285076 | 4.431005765 | 4.113932028 | 4.69344434 | 3.655750515 | 4.042241667 | 4.044894771 |
| hsa-miR-600_st | 5.465360277 | 5.660066921 | 4.725298299 | 4.571524582 | 5.416411683 | 5.526296346 | 4.043056928 |
| hsa-miR-601_st | 5.627316069 | 6.089278354 | 4.697206951 | 4.791263503 | 6.524982459 | 5.948292481 | 5.524552246 |
| hsa-miR-602_st | 13.15090327 | 12.89815612 | 12.19107695 | 13.07171216 | 13.04329751 | 14.03002919 | 13.63949319 |
| hsa-miR-603_st | 3.536959524 | 1.875330278 | 2.419784634 | 2.371923729 | 2.375770845 | 0.386410684 | 0.728060881 |
| hsa-miR-604_st | 8.273628962 | 8.061633592 | 8.370017987 | 8.984732027 | 8.032766566 | 9.465384586 | 9.203509967 |
| hsa-miR-605_st | 6.137813329 | 5.212763587 | 5.27613191 | 3.435614661 | 6.17463945 | 5.852856363 | 5.206011824 |
| hsa-miR-606_st | 1.617429852 | 2.102062071 | 2.632477803 | 2.525372986 | 1.250412158 | 1.506709577 | 1.482482286 |
| hsa-miR-607_st | 2.343004256 | 2.994529175 | 2.635818339 | 3.118019176 | 1.418068623 | 2.583979837 | 2.092793802 |
| hsa-miR-608_st | 8.877024008 | 9.062934221 | 8.919377674 | 9.074302895 | 9.379407418 | 9.463746519 | 9.499801629 |
| hsa-miR-609_st | 2.84304626 | 2.923296145 | 2.424579692 | 2.24317475 | 2.406284424 | 1.390437928 | 1.49318755 |
| hsa-miR-610_st | 5.274501112 | 4.556253312 | 5.635909227 | 5.269175438 | 5.008994342 | 4.860049194 | 4.895667052 |
| hsa-miR-611_st | 7.633488225 | 6.215205355 | 7.146574667 | 6.797561515 | 7.623934664 | 8.166107899 | 7.352309843 |
| hsa-miR-612_st | 11.54291563 | 10.16311536 | 10.91953569 | 10.90287527 | 11.73181419 | 11.89999505 | 11.82806173 |
| hsa-miR-613_st | 3.62040995 | 3.841091303 | 4.625743676 | 4.080908351 | 4.366156616 | 3.388841376 | 3.413393802 |
| hsa-miR-614_st | 5.812001508 | 5.437330343 | 4.473376578 | 5.494611952 | 5.700395066 | 6.643097642 | 6.307541562 |
| hsa-miR-615-5p_st | 10.57918769 | 9.833331791 | 9.7623915 | 9.883754278 | 10.54857436 | 10.98987694 | 10.93498938 |
| hsa-miR-615-3p_st | 7.573934471 | 6.138085639 | 6.205335604 | 7.730413305 | 6.886265114 | 6.976313035 | 6.841559235 |
| hsa-miR-616-star_st | 4.499478979 | 4.656314984 | 3.985019577 | 4.253940162 | 4.802356869 | 3.443595469 | 3.436301985 |
| hsa-miR-616_st | 7.600061873 | 6.773423822 | 6.877939268 | 6.323385517 | 7.94386325 | 7.031291407 | 7.170596487 |
| hsa-miR-548c-5p_st | 8.625708585 | 8.118960159 | 8.707674975 | 7.664363464 | 8.655909164 | 6.318980618 | 6.003063976 |
| hsa-miR-548c-3p_st | 1.110706338 | 1.328843738 | 0.4863786 | 0.666563166 | 0.992594083 | 0.327774959 | 1.101654274 |
| hsa-miR-617_st | 5.302938397 | 5.569821714 | 5.018231274 | 5.390432001 | 6.811839293 | 5.48495218 | 5.964345857 |
| hsa-miR-618_st | 3.182785655 | 2.898692146 | 2.850006572 | 3.503150818 | 4.305498338 | 1.794159357 | 3.48606908 |
| hsa-miR-619_st | 3.665468574 | 3.785546152 | 3.683794292 | 3.083758024 | 3.818686757 | 3.909754737 | 3.707512244 |
| hsa-miR-620_st | 2.44025913 | 3.040608004 | 2.026886071 | 2.181694881 | 3.005367974 | 2.318672573 | 2.464278035 |
| hsa-miR-621_st | 3.719083689 | 3.382126279 | 3.217000018 | 2.541344354 | 3.440252944 | 2.936676434 | 3.194331179 |
| hsa-miR-622_st | 8.642610505 | 8.166661704 | 8.616923443 | 9.043206419 | 9.285747076 | 9.380614396 | 8.913270568 |
| hsa-miR-623_st | 8.569399854 | 8.040030533 | 7.952447719 | 8.440211545 | 8.306041408 | 9.517905604 | 8.912827573 |
| hsa-miR-624-star_st | 4.291064737 | 3.883820202 | 3.428780769 | 2.978121524 | 3.792996738 | 3.699412479 | 3.094544016 |
| hsa-miR-624_st | 2.978046143 | 2.796435163 | 1.787087296 | 2.458950893 | 1.411996277 | 2.661219971 | 2.692581166 |
| hsa-miR-625_st | 6.871307101 | 6.527948101 | 7.297107814 | 6.469159169 | 6.831217039 | 6.524150093 | 6.21824841 |
| hsa-miR-625-star_st | 4.576422678 | 3.018605998 | 3.802362362 | 2.797049012 | 3.678003952 | 3.870741947 | 3.835916538 |
| hsa-miR-626_st | 0.714596607 | 1.775193777 | 2.459703882 | 2.305714218 | 2.55770444 | 2.088218522 | 1.363835842 |
| hsa-miR-627_st | 1.431712781 | 1.657261287 | 1.32251047 | 1.173781862 | 0.283398022 | 0.595399103 | 1.609555357 |
| hsa-miR-628-5p_st | 1.856465736 | 1.958409645 | 2.990632861 | 2.240612964 | 2.235299217 | 2.182061249 | 2.17672931 |
| hsa-miR-628-3p_st | 9.125401099 | 8.349944068 | 9.598077304 | 9.829492208 | 10.10309557 | 10.05817015 | 9.874305754 |
| hsa-miR-629_st | 7.225504218 | 7.745354517 | 7.834408609 | 8.627683281 | 6.861469686 | 7.641208121 | 7.603599999 |
| hsa-miR-629-star_st | 5.748524211 | 5.243198805 | 5.156274568 | 5.352960855 | 5.313647678 | 4.992653128 | 4.481839114 |
| hsa-miR-630_st | 6.485579016 | 5.428255658 | 5.945747534 | 5.278760897 | 6.916335845 | 7.976016256 | 6.973217804 |
| hsa-miR-631_st | 6.422610303 | 5.74323599 | 5.979011349 | 5.772490551 | 6.204126669 | 6.763590719 | 5.880281471 |
| hsa-miR-33b_st | 1.594267299 | 1.134749481 | 1.380759324 | 1.660417239 | 0.656050831 | 0.219507921 | −0.325445503 |
| hsa-miR-33b-star_st | 6.427703381 | 5.726989374 | 6.072573525 | 5.934780525 | 6.172740449 | 6.912862072 | 6.720499911 |
| hsa-miR-632_st | 4.26307099 | 3.384095517 | 4.938680281 | 4.578246658 | 4.282751828 | 3.32925136 | 3.40745619 |
| hsa-miR-633_st | 0.891478443 | 2.429348457 | 0.954422618 | 1.40576831 | 1.668639943 | 1.493379171 | 1.561699059 |
| hsa-miR-634_st | 5.860920829 | 5.810171457 | 5.812635519 | 5.353943851 | 7.020365158 | 4.471178928 | 4.969976182 |
| hsa-miR-635_st | 4.533979501 | 5.259604756 | 3.85885678 | 4.984546224 | 5.196680422 | 4.21820803 | 3.859246708 |
| hsa-miR-636_st | 8.946469281 | 8.384034046 | 7.523284321 | 8.635086475 | 8.679252266 | 9.676805929 | 9.431240623 |
| hsa-miR-637_st | 12.23726549 | 11.73601894 | 11.38069999 | 11.7179655 | 11.99334681 | 12.50436026 | 12.47145251 |
| hsa-miR-638_st | 15.51481568 | 16.2473256 | 15.31212226 | 15.49869309 | 14.53321459 | 15.83136002 | 15.71068299 |
| hsa-miR-639_st | 8.292126969 | 7.832611502 | 7.463580326 | 8.2048689 | 8.693443021 | 9.020292548 | 8.72419512 |
| hsa-miR-640_st | 6.676006823 | 6.475776455 | 5.524015524 | 6.274460174 | 6.49079198 | 6.451735732 | 7.041966752 |
| hsa-miR-641_st | 3.622857483 | 3.311325204 | 3.073800633 | 2.892273963 | 3.81889203 | 2.866898993 | 3.486859931 |
| hsa-miR-642_st | 3.594145741 | 3.542538841 | 3.323983635 | 3.310550527 | 4.741299326 | 2.742469227 | 4.115737103 |
| hsa-miR-643_st | 1.88652457 | 1.616446441 | 2.238630498 | 2.506634027 | 1.824814819 | 2.110146211 | 1.952078318 |
| hsa-miR-644_st | 2.437943686 | 2.128516964 | 2.67245495 | 1.184166214 | 2.281244501 | 1.683423942 | 0.63748629 |
| hsa-miR-645_st | 2.949351982 | 2.12732061 | 2.61177189 | 3.461361109 | 2.570170393 | 3.665325806 | 2.453147856 |
| hsa-miR-646_st | 2.942679033 | 3.592030927 | 2.8908041 | 3.294284003 | 2.816545338 | 2.570405234 | 3.449108798 |
| hsa-miR-647_st | 6.077940692 | 5.067515565 | 4.503260575 | 5.309295877 | 5.67896594 | 4.392055873 | 4.685470695 |
| hsa-miR-648_st | 7.279707849 | 7.185651907 | 6.970137345 | 6.50069676 | 7.243415957 | 7.72502138 | 7.886615472 |
| hsa-miR-649_st | 4.905834189 | 4.087821649 | 5.239081728 | 4.629922998 | 5.240424457 | 2.618649422 | 2.773472447 |
| hsa-miR-650_st | 10.67361231 | 9.154659886 | 9.987968893 | 10.01782265 | 10.85943975 | 11.17961851 | 10.19646105 |
| hsa-miR-651_st | 0.31611231 | 0.798286945 | 0.583001679 | −0.014842326 | 0.143785414 | 0.972959323 | 1.952648617 |
| hsa-miR-652_st | 7.450767149 | 7.03317083 | 8.24475439 | 7.599168124 | 7.674285338 | 6.604032907 | 8.339292538 |
| hsa-miR-548d-5p_st | 7.524343032 | 6.484600725 | 7.939994823 | 6.244845357 | 7.762601635 | 5.718696533 | 4.639829452 |
| hsa-miR-548d-3p_st | 1.636809693 | 2.526720726 | 2.789680332 | 2.296207428 | 1.893897802 | 1.285495338 | 1.890100929 |
| hsa-miR-661_st | 7.612861528 | 6.413613372 | 6.69074105 | 6.659990028 | 7.614260745 | 7.298928723 | 6.558911334 |
| hsa-miR-662_st | 6.11976633 | 6.44608912 | 6.76443855 | 5.926590827 | 6.08841694 | 6.301181394 | 6.269652085 |
| hsa-miR-663_st | 15.22126025 | 15.6519729 | 15.04527593 | 15.40562496 | 14.53350071 | 15.83052149 | 15.57890658 |
| hsa-miR-449b_st | 4.676952894 | 5.299151435 | 4.921417799 | 4.594422 | 5.025669722 | 4.585845192 | 4.748592436 |
| hsa-miR-449b-star_st | 7.674329433 | 7.239350928 | 7.137335961 | 7.997913975 | 7.693145608 | 7.495554124 | 7.364270467 |
| hsa-miR-653_st | 0.29098795 | 1.830693654 | 0.207961931 | 1.872402808 | −0.66983009 | 0.306524379 | 0.853540575 |
| hsa-miR-411_st | 3.675302593 | 3.02096103 | 2.962308284 | 3.419316101 | 2.480368443 | 3.454803377 | 4.974721301 |
| hsa-miR-411-star_st | 3.585204222 | 3.769389512 | 4.094779996 | 3.867622112 | 4.153818767 | 3.85823412 | 3.672295845 |
| hsa-miR-654-5p_st | 10.89687384 | 9.956949848 | 9.548772041 | 9.903340007 | 10.51933652 | 11.32770137 | 11.28177457 |
| hsa-miR-654-3p_st | 3.529801042 | 2.867773379 | 3.63431131 | 2.70532274 | 4.321342493 | 5.233089123 | 5.712914245 |
| hsa-miR-655_st | 0.683665929 | 1.444516254 | 1.261843693 | 1.540178957 | 0.355206552 | 0.998566646 | 0.611942618 |
| hsa-miR-656_st | 2.483428501 | 2.553678605 | 2.580217036 | 2.386504193 | 2.20237361 | 1.458517042 | 2.02155612 |
| hsa-miR-549_st | 2.565386727 | 2.26205593 | 2.667043567 | 2.383707078 | 2.297376334 | 2.211233738 | 2.280250463 |
| hsa-miR-657_st | 4.285194365 | 4.329163626 | 4.262950773 | 3.530581224 | 3.682643693 | 3.731013551 | 4.554679608 |
| hsa-miR-658_st | 11.76238903 | 11.37030017 | 11.37636624 | 11.36001273 | 11.12683658 | 11.97473325 | 12.2603194 |
| hsa-miR-659_st | 10.78477061 | 9.689560107 | 9.69071639 | 10.00967777 | 10.20855818 | 11.08369834 | 10.73305553 |
| hsa-miR-660_st | 6.91351322 | 7.165370671 | 8.217642038 | 7.915878655 | 6.631565089 | 5.944121769 | 6.208002101 |
| hsa-miR-421_st | 6.292083739 | 6.541192276 | 7.040990232 | 7.256618274 | 6.24272431 | 5.911003491 | 6.983259903 |
| hsa-miR-542-5p_st | 7.538601115 | 7.430618497 | 7.483102683 | 8.738682299 | 7.125946488 | 7.135160862 | 7.517791184 |
| hsa-miR-542-3p_st | 5.992649971 | 5.427924897 | 6.184252194 | 7.740176207 | 6.378661276 | 6.016607322 | 5.221555897 |
| hsa-miR-758_st | 2.356753045 | 1.915534234 | 0.987204286 | 2.981493291 | 3.452297929 | 3.397588165 | 3.612410114 |
| hsa-miR-1264_st | 2.373729404 | 2.668659426 | 3.031760709 | 2.651096276 | 4.186456349 | 1.894568394 | 2.040723758 |
| hsa-miR-671-5p_st | 11.2166394 | 10.50533991 | 10.76897197 | 10.62282309 | 11.14212132 | 12.10912696 | 11.67985263 |
| hsa-miR-671-3p_st | 6.76424341 | 6.527312033 | 6.795322889 | 5.364176729 | 6.791221173 | 7.066787233 | 7.308369973 |
| hsa-miR-668_st | 8.166866106 | 7.541167752 | 7.537283571 | 7.573939927 | 7.783709078 | 8.122397637 | 8.497071384 |
| hsa-miR-767-5p_st | 3.093920954 | 3.059321236 | 2.318376524 | 2.742392556 | 3.885189876 | 2.94276661 | 2.336666396 |
| hsa-miR-767-3p_st | 2.907468673 | 2.146622261 | 2.765413149 | 3.423100097 | 3.569948578 | 2.661450064 | 1.625299882 |
| hsa-miR-1224-5p_st | 12.63962093 | 12.30011815 | 12.10505473 | 12.48549894 | 12.65197772 | 13.81310379 | 13.41424396 |
| hsa-miR-1224-3p_st | 5.148364882 | 5.067414265 | 5.025677061 | 5.312534298 | 5.901404217 | 5.305855073 | 6.110508956 |
| hsa-miR-320b_st | 11.34828284 | 11.0320645 | 11.83049238 | 11.08969333 | 10.94293063 | 10.95823093 | 11.0179839 |
| hsa-miR-320c_st | 10.83115587 | 10.79043387 | 11.40979838 | 10.81243036 | 9.846536697 | 10.6228549 | 10.54496136 |
| hsa-miR-1296_st | 6.94929788 | 6.219152246 | 5.926965222 | 7.1215218 | 6.34744579 | 6.706243975 | 6.574410089 |
| hsa-miR-1468_st | 5.728014571 | 5.152276148 | 4.48270612 | 5.432003774 | 6.106457987 | 5.790944847 | 6.098629129 |
| hsa-miR-1323_st | 7.296208168 | 7.151319376 | 6.560656612 | 7.365533388 | 7.559942946 | 7.756625817 | 6.946026848 |
| hsa-miR-1271_st | 6.686845344 | 6.467967216 | 6.574415452 | 6.706215066 | 7.065287613 | 7.20091514 | 7.119404754 |
| hsa-miR-1301_st | 8.084569985 | 7.108185328 | 7.578494602 | 8.017643584 | 8.055483672 | 8.629387415 | 8.414236051 |
| hsa-miR-454-star_st | 1.239092419 | 2.077953993 | 1.657948294 | 3.337385718 | 1.27070949 | 0.976904255 | 2.045022086 |
| hsa-miR-454_st | 4.176676364 | 4.753369128 | 6.794563918 | 6.533331517 | 3.749690497 | 4.104854563 | 4.966299313 |
| hsa-miR-1185_st | 0.826857153 | 1.192026612 | 1.151480351 | 0.544278827 | 0.286042437 | 0.450648889 | 0.979244433 |
| hsa-miR-449c_st | 6.645129375 | 5.970884399 | 5.436798472 | 5.661979285 | 6.889171256 | 7.531881047 | 6.04504776 |
| hsa-miR-449c-star_st | 3.539802311 | 3.387327209 | 3.629923134 | 3.82373556 | 4.607836709 | 2.910863582 | 3.13177466 |
| hsa-miR-1283_st | 1.789740947 | 2.204471677 | 1.736329224 | 2.884920933 | 2.94052832 | 1.637115039 | 2.363002158 |
| hsa-miR-769-5p_st | 6.760567422 | 6.75614509 | 7.890262132 | 6.251294397 | 6.814134375 | 7.274034493 | 6.888648694 |
| hsa-miR-769-3p_st | 9.516922051 | 8.702776821 | 8.855037655 | 8.849180626 | 9.280707277 | 9.643072794 | 9.585431556 |
| hsa-miR-766_st | 7.884016592 | 7.4793299 | 7.315786363 | 7.636174948 | 7.955034501 | 7.976051989 | 7.344995225 |
| hsa-miR-762_st | 15.32478604 | 15.96368556 | 15.29530767 | 15.49841851 | 14.53399291 | 15.83120318 | 15.70978622 |
| hsa-miR-802_st | 1.152737519 | 1.736079043 | −0.026235431 | 0.079138161 | −0.157472091 | 0.095142909 | 1.430155661 |
| hsa-miR-670_st | 4.38787298 | 4.136177353 | 5.041062019 | 4.611749446 | 4.618876664 | 3.699027129 | 4.315760354 |
| hsa-miR-1298_st | 4.030354023 | 3.874375872 | 3.952001842 | 4.002448406 | 4.510695602 | 3.886093541 | 4.364631887 |
| hsa-miR-2113_st | 6.210884603 | 5.657316496 | 5.301636981 | 5.50045067 | 5.665891555 | 5.394681433 | 5.167143478 |
| hsa-miR-761_st | 7.353817523 | 6.477147623 | 6.964975682 | 7.149223486 | 7.882177289 | 7.564286267 | 7.249536399 |
| hsa-miR-764_st | 5.85455759 | 4.925197354 | 5.375425594 | 5.15940143 | 6.039439423 | 5.775061059 | 5.357170518 |
| hsa-miR-759_st | 1.055534708 | 1.966177208 | 1.118481697 | 1.922079508 | 1.264361104 | 2.818703892 | −0.334985355 |
| hsa-miR-765_st | 7.935969438 | 8.066777002 | 8.777468432 | 8.734867122 | 9.303377607 | 8.882514331 | 9.368641279 |
| hsa-miR-770-5p_st | 6.60960487 | 6.199135255 | 5.9966288 | 6.449524499 | 6.594717781 | 6.485427675 | 5.860416893 |
| hsa-miR-675_st | 13.19734892 | 12.49017431 | 12.64268068 | 12.58654091 | 12.85752223 | 13.69886219 | 13.42561372 |
| hsa-miR-675-star_st | 6.349648086 | 6.301195437 | 5.325734291 | 6.864306949 | 7.01236302 | 7.839194365 | 6.504058662 |
| hsa-miR-298_st | 9.86200511 | 9.243125847 | 9.503658689 | 10.16355024 | 9.842704559 | 10.68087216 | 10.50549213 |
| hsa-miR-891a_st | 6.485438184 | 5.998112292 | 6.343441571 | 5.660959262 | 6.448875055 | 6.128087718 | 8.623106865 |
| hsa-miR-300_st | 5.214506346 | 4.772604338 | 4.67253437 | 4.499143644 | 5.495126266 | 4.840401035 | 5.231660564 |
| hsa-miR-886-5p_st | 11.75223698 | 11.57501668 | 10.56355167 | 12.10515393 | 11.01041386 | 12.14903466 | 12.12630147 |
| hsa-miR-886-3p_st | 9.641649359 | 9.994555728 | 9.518346438 | 11.31319403 | 8.967143143 | 11.01698561 | 10.33262396 |
| hsa-miR-892a_st | 4.729286441 | 3.738034823 | 3.640155623 | 3.585331961 | 4.670721249 | 2.651455468 | 5.049494031 |
| hsa-miR-220b_st | 3.707205793 | 3.59884387 | 3.547979065 | 3.073998958 | 3.715838095 | 2.564431385 | 3.120227978 |
| hsa-miR-450b-5p_st | 0.676718281 | 1.021609875 | 0.705111228 | −0.134594011 | 2.114140843 | 0.309320889 | −0.029960092 |
| hsa-miR-450b-3p_st | 0.951900349 | 1.64388678 | 0.48353567 | 0.171237495 | 0.756576061 | 0.461603648 | 0.288112462 |
| hsa-miR-874_st | 11.98068336 | 11.27985073 | 11.93202627 | 11.73980948 | 11.93944802 | 12.77845115 | 12.17761583 |
| hsa-miR-890_st | 6.15054768 | 5.855085113 | 5.837506751 | 6.050968077 | 6.185355794 | 5.596077845 | 7.181132662 |
| hsa-miR-891b_st | 3.122597921 | 3.288394888 | 3.694607668 | 2.762275343 | 2.440615367 | 3.051339285 | 5.018197032 |
| hsa-miR-220c_st | 6.07010339 | 4.910335716 | 6.1408178 | 5.753228908 | 6.993546786 | 5.866489448 | 5.999797051 |
| hsa-miR-888_st | 1.696069021 | 3.018200648 | 1.576282828 | 0.563992224 | 2.307508038 | 0.994823711 | 4.1520701 |
| hsa-miR-888-star_st | 1.377590098 | 0.59327829 | 1.351050351 | 1.620999064 | 0.647062406 | 0.871178503 | 1.16920787 |
| hsa-miR-892b_st | 4.261052098 | 4.079689762 | 4.672374113 | 4.610072906 | 5.183545378 | 2.912148696 | 5.048497881 |
| hsa-miR-541-star_st | 5.748402412 | 5.981574759 | 5.681589828 | 6.2433838 | 6.341800525 | 5.9391511 | 6.093392763 |
| hsa-miR-541_st | 7.639489072 | 7.513571252 | 6.779326801 | 7.50476707 | 8.232493466 | 8.101263457 | 7.944180247 |
| hsa-miR-889_st | 1.585279305 | 1.192503034 | 1.72674327 | 1.342641786 | 2.626353029 | 2.173169019 | 1.639130402 |
| hsa-miR-875-5p_st | 0.538540167 | −0.011661196 | 0.254664596 | 0.251640439 | 0.166005299 | −0.330084559 | −0.70968401 |
| hsa-miR-875-3p_st | 3.163147417 | 3.228995953 | 3.385398059 | 2.536006385 | 3.571982174 | 2.958475739 | 3.339702984 |
| hsa-miR-876-5p_st | 0.594502261 | −0.355354001 | −0.652631534 | −0.3947282 | −0.3768045 | 0.389231182 | 0.975598963 |
| hsa-miR-876-3p_st | 1.357905334 | 1.304829813 | 1.800715892 | 0.480081216 | 1.643916906 | 1.470525506 | 1.595257862 |
| hsa-miR-708_st | 7.55440396 | 6.27260111 | 6.17902355 | 7.07138296 | 7.474919194 | 8.076530152 | 5.52680611 |
| hsa-miR-708-star_st | 4.373964346 | 4.004395469 | 3.584263559 | 3.609237171 | 3.887428614 | 3.154803137 | 2.345708843 |
| hsa-miR-147b_st | 4.371331162 | 3.722308953 | 4.297558583 | 3.476675556 | 4.227044978 | 3.738034141 | 4.063454842 |
| hsa-miR-190b_st | 0.279410031 | 0.566346156 | 0.088421212 | −0.30435167 | −0.622722797 | −0.656747747 | −0.385496095 |
| hsa-miR-744_st | 14.09324847 | 14.01963807 | 13.64159611 | 14.06808712 | 14.11701445 | 14.72129845 | 14.68870069 |
| hsa-miR-744-star_st | 4.45847073 | 3.91925144 | 4.745799792 | 4.133613416 | 4.936226466 | 4.287916966 | 4.59136773 |
| hsa-miR-885-5p_st | 6.965484739 | 6.116181877 | 6.00167497 | 5.707053667 | 8.165501697 | 7.495485076 | 6.150788281 |
| hsa-miR-885-3p_st | 13.2093057 | 12.94536002 | 12.2653855 | 12.90503522 | 12.66575473 | 13.82107055 | 13.49329099 |
| hsa-miR-877_st | 11.46354785 | 11.78968278 | 10.71341474 | 11.34637974 | 11.2419919 | 12.09846103 | 12.13112183 |
| hsa-miR-877-star_st | 6.90200244 | 5.877473647 | 6.850534177 | 6.370116962 | 6.836094512 | 7.077277729 | 7.217048327 |
| hsa-miR-887_st | 7.591891047 | 7.944165668 | 7.776728422 | 8.169008912 | 8.469177497 | 9.67007441 | 9.004490368 |
| hsa-miR-665_st | 12.47362751 | 11.88170737 | 11.75587876 | 11.66010024 | 12.31264344 | 13.07547905 | 12.75831744 |
| hsa-miR-873_st | 5.07787817 | 3.111169468 | 5.331925886 | 4.696980553 | 4.987182193 | 4.535719474 | 4.284712641 |
| hsa-miR-543_st | 1.805540682 | 3.693977546 | 3.307393455 | 3.521693424 | 4.092543664 | 3.606751033 | 5.09122863 |
| hsa-miR-374b_st | 4.122166731 | 6.194190776 | 7.073874094 | 7.739798099 | 6.291447371 | 4.270283018 | 6.490112402 |
| hsa-miR-374b-star_st | 2.319429497 | 2.237518698 | 2.30828274 | 2.011073851 | 1.13480868 | 1.271432344 | 2.111220352 |
| hsa-miR-760_st | 10.26386087 | 9.848326177 | 9.272927986 | 9.748738274 | 9.917078607 | 10.63963506 | 10.26605492 |
| hsa-miR-301b_st | 0.231558773 | 1.179847703 | 2.32644415 | 1.442854696 | 0.253975495 | 1.485389445 | −0.076167876 |
| hsa-miR-216b_st | 3.224938778 | 4.289049403 | 4.286144411 | 4.872684375 | 3.461247613 | 3.114670074 | 4.23896913 |
| hsa-miR-208b_st | 2.994767965 | 2.966718509 | 2.985520328 | 3.146335298 | 2.496993364 | 2.61246972 | 2.549902803 |
| hsa-miR-920_st | 10.25743035 | 9.248871646 | 9.285180179 | 9.892453413 | 10.03615941 | 10.4941513 | 10.56397822 |
| hsa-miR-921_st | 10.72670217 | 10.02921398 | 10.17211297 | 10.3213818 | 11.05128032 | 11.22969542 | 11.05397654 |
| hsa-miR-922_st | 5.84945184 | 4.903436763 | 4.66302554 | 5.630938246 | 6.118060539 | 5.455006984 | 4.695791623 |
| hsa-miR-924_st | 2.491867963 | 3.064888593 | 3.72012057 | 2.751335896 | 3.24106063 | 0.944424102 | 1.327009845 |
| hsa-miR-509-3-5p_st | 5.704604353 | 5.721052794 | 5.213916472 | 4.430116846 | 6.389521076 | 6.070772308 | 5.921931979 |
| hsa-miR-933_st | 10.75343369 | 10.80442101 | 10.57256457 | 10.84547546 | 10.38957675 | 11.13175078 | 11.07831093 |
| hsa-miR-934_st | 3.250592731 | 2.835204132 | 4.01856052 | 2.354720365 | 3.974389046 | 3.808497332 | 3.790059051 |
| hsa-miR-935_st | 5.895680033 | 6.086855177 | 4.990401387 | 5.295087922 | 6.102459821 | 6.513293001 | 6.632482008 |
| hsa-miR-936_st | 9.816728836 | 9.717692655 | 9.225127815 | 9.658492954 | 9.865199903 | 10.93795777 | 10.81893123 |
| hsa-miR-937_st | 4.901800791 | 4.2046582 | 4.663566531 | 4.626611085 | 5.097318377 | 5.239588125 | 5.249526816 |
| hsa-miR-938_st | 4.449343942 | 4.031007287 | 3.796089877 | 3.811024811 | 4.095061408 | 4.673627389 | 3.99445415 |
| hsa-miR-939_st | 13.08393623 | 12.64033484 | 12.03966903 | 12.73761373 | 12.67205064 | 13.81120354 | 13.40577753 |
| hsa-miR-940_st | 7.802836338 | 7.609601727 | 7.821727117 | 7.217288858 | 8.522792748 | 8.615252218 | 8.327456171 |
| hsa-miR-941_st | 5.326987922 | 5.36717838 | 4.823248685 | 5.803131396 | 5.002156186 | 5.652529123 | 4.87666116 |
| hsa-miR-942_st | 1.461599964 | 2.015805405 | 2.340303697 | 1.60638432 | 2.395610188 | 1.401256373 | 1.623535994 |
| hsa-miR-943_st | 6.921388551 | 6.882490198 | 6.317256362 | 7.556737717 | 7.500512109 | 7.99479988 | 7.662945752 |
| hsa-miR-944_st | 1.385904372 | 1.831466572 | 2.126535001 | 1.250160667 | 1.713341953 | 1.060340904 | 0.418168653 |
| hsa-miR-297_st | 5.404786268 | 5.250557885 | 5.758845309 | 6.011798969 | 6.109259778 | 4.110748171 | 4.41850846 |
| hsa-miR-1178_st | 3.770267416 | 3.53003298 | 3.720843102 | 3.632374942 | 4.125609452 | 3.767587811 | 3.923467427 |
| hsa-miR-1179_st | 0.560924876 | 1.421815892 | 1.771973998 | 0.845535599 | 1.795442517 | 0.712885228 | 1.201206129 |
| hsa-miR-1180_st | 9.421351806 | 8.958293968 | 9.070844136 | 9.512605685 | 8.770750042 | 9.619220857 | 9.300269595 |
| hsa-miR-1181_st | 10.1667549 | 9.257919496 | 9.121851101 | 9.254555289 | 9.520597234 | 9.881645829 | 9.281577198 |
| hsa-miR-1182_st | 10.3205141 | 9.936479523 | 9.957470533 | 10.51538306 | 10.21864216 | 10.79130674 | 10.80056654 |
| hsa-miR-1183_st | 10.23912762 | 10.04171726 | 9.992086591 | 10.01354395 | 10.56144488 | 10.69011981 | 10.31387753 |
| hsa-miR-1184_st | 4.352302368 | 3.261210089 | 4.057078066 | 4.01028496 | 5.606830423 | 3.49055658 | 4.204705624 |
| hsa-miR-1225-5p_st | 13.3730496 | 13.48241304 | 12.82226514 | 13.59285649 | 13.39780002 | 14.52703283 | 14.17282982 |
| hsa-miR-1225-3p_st | 7.768562129 | 7.267447194 | 6.729967449 | 7.452748265 | 7.133931882 | 8.361052227 | 7.858234309 |
| hsa-miR-1226-star_st | 10.7835261 | 10.23493658 | 10.05988077 | 10.52565585 | 10.66117382 | 11.5311234 | 11.45877427 |
| hsa-miR-1226_st | 5.708953349 | 5.353960481 | 5.216292366 | 6.034400493 | 6.337818284 | 6.310487711 | 6.036169071 |
| hsa-miR-1227_st | 5.888694077 | 4.339955243 | 5.325032501 | 5.290556798 | 6.239850805 | 5.432499317 | 5.318159461 |
| hsa-miR-1228-star_st | 15.24867146 | 15.88335068 | 15.1705223 | 15.44423905 | 14.53349253 | 15.83167904 | 15.67048775 |
| hsa-miR-1228_st | 7.832604424 | 7.072740162 | 7.354228815 | 8.270985887 | 8.516011351 | 8.869672515 | 7.901490459 |
| hsa-miR-1229_st | 8.079337925 | 7.502404518 | 7.112456326 | 7.695573252 | 7.596396217 | 7.733745443 | 7.03622041 |
| hsa-miR-1231_st | 13.29546515 | 13.14347679 | 12.33068351 | 13.32037528 | 12.99435053 | 14.44250904 | 13.94326103 |
| hsa-miR-1233_st | 6.727736604 | 6.347853318 | 6.269290876 | 7.117139428 | 7.133041061 | 7.514199728 | 7.609595002 |
| hsa-miR-1234_st | 7.446542778 | 6.2070202 | 6.916643802 | 6.335625772 | 7.005074887 | 7.426480601 | 6.756259179 |
| hsa-miR-1236_st | 5.525625644 | 5.60077085 | 5.227504529 | 4.979727201 | 6.166442988 | 5.479618794 | 6.111388293 |
| hsa-miR-1237_st | 7.460143651 | 7.254932199 | 7.061362775 | 7.244196443 | 7.645789754 | 8.371892233 | 7.809468726 |
| hsa-miR-1238_st | 6.497894829 | 6.285586177 | 6.297129236 | 6.399280834 | 6.940816489 | 7.549483489 | 7.17850241 |
| hsa-miR-1200_st | 5.205927165 | 4.891564155 | 4.646726015 | 3.77347417 | 5.811080719 | 4.571752641 | 4.009752348 |
| hsa-miR-1201_st | 8.623426156 | 8.152476457 | 8.990078198 | 8.594043609 | 9.30514718 | 8.614697629 | 8.734646149 |
| hsa-miR-1202_st | 12.63747686 | 12.20918987 | 12.18939523 | 12.53055799 | 12.54326328 | 13.4332898 | 13.24944402 |
| hsa-miR-1203_st | 7.996504376 | 7.891752331 | 7.710798648 | 8.052820543 | 8.893491702 | 9.112853704 | 9.125542166 |
| hsa-miR-663b_st | 10.40662859 | 9.915337424 | 9.714881182 | 9.332496274 | 10.42737433 | 10.97972878 | 10.37230224 |
| hsa-miR-1204_st | 5.304191098 | 4.337908484 | 4.408029091 | 5.328821729 | 5.227907626 | 5.041018778 | 4.588724561 |
| hsa-miR-1205_st | 3.347458996 | 3.12734715 | 3.383574857 | 4.384720258 | 4.1870661 | 3.412034655 | 4.025876692 |
| hsa-miR-1206_st | 0.824532418 | 1.57223996 | 0.657986719 | 1.048979327 | 2.288892355 | 0.845189422 | 1.480204668 |
| hsa-miR-1207-5p_st | 14.50651391 | 14.6557507 | 14.05413455 | 14.61926619 | 14.24945829 | 15.40218271 | 15.15191042 |
| hsa-miR-1207-3p_st | 4.199337067 | 3.08812303 | 2.945595881 | 3.872048223 | 3.409220845 | 3.704736501 | 4.075857993 |
| hsa-miR-1208_st | 7.899113283 | 7.237238637 | 7.038563395 | 7.737828848 | 8.266511833 | 9.426295937 | 8.532575206 |
| hsa-miR-548 [symbol] _st | 0.44686361 | 0.683115974 | 0.533342237 | 2.542039214 | 0.81914677 | 0.47013454 | 0.946564208 |
| hsa-miR-548 [symbol] _st | 4.1487535 | 2.891615064 | 3.507354881 | 3.231510925 | 3.876967683 | 2.270491356 | 2.524108531 |
| hsa-miR-1285_st | 13.33430111 | 13.00815091 | 12.4887947 | 12.57602047 | 12.82914766 | 12.98824106 | 13.12505705 |
| hsa-miR-1286_st | 8.741030173 | 7.03040932 | 7.896966469 | 8.102601477 | 8.89751111 | 8.911098633 | 8.880238809 |
| hsa-miR-1287_st | 5.779484489 | 5.685263222 | 6.054232493 | 6.058445011 | 6.864395644 | 5.375984243 | 6.575840604 |
| hsa-miR-1289_st | 5.964265517 | 4.520060915 | 5.507904402 | 5.094374561 | 5.445014135 | 3.936551758 | 4.522606497 |
| hsa-miR-1290_st | 9.166036374 | 8.582298339 | 8.994859989 | 9.041258483 | 9.498916604 | 9.456733878 | 9.276381837 |
| hsa-miR-1291_st | 7.735000403 | 7.80364345 | 8.381709846 | 8.477314751 | 7.995232471 | 8.101655928 | 8.341013114 |
| hsa-miR-548k_st | 1.884914027 | 2.941338154 | 3.571631226 | 3.845117167 | 2.46458227 | 11.544746147 | 1.392997582 |
| hsa-miR-1293_st | 7.647704336 | 7.266265779 | 7.421994767 | 7.468327567 | 7.782436481 | 7.457729481 | 7.689659567 |
| hsa-miR-1294_st | 5.758069726 | 5.289270136 | 6.084272202 | 4.969828464 | 5.464682568 | 4.906513629 | 5.702112521 |
| hsa-miR-1295_st | 5.842828641 | 5.498166572 | 5.04366399 | 5.094810736 | 5.914544071 | 6.197025204 | 6.405333717 |
| hsa-miR-1297_st | 0.799085434 | 1.773753189 | 1.474034948 | −0.154347372 | 0.046134513 | 1.630350888 | 0.903267171 |
| hsa-miR-1299_st | 6.333317555 | 4.846896731 | 5.70384078 | 5.737263448 | 6.914061367 | 4.31423626 | 3.655186293 |
| hsa-miR-548l_st | 4.009002081 | 3.809654632 | 4.133619263 | 4.560570934 | 4.507167452 | 3.367278831 | 4.353228874 |
| hsa-miR-1302_st | 1.652386045 | 2.051516906 | 1.297898644 | 0.740963765 | 1.621003123 | 2.030055038 | 1.725563732 |
| hsa-miR-1303_st | 10.75840089 | 10.31014521 | 9.987055503 | 9.641007274 | 9.872577705 | 10.3979247 | 10.28600451 |
| hsa-miR-1304_st | 6.486539798 | 6.575388847 | 6.761035036 | 6.266261892 | 7.212002475 | 6.581001318 | 7.291395156 |
| hsa-miR-1305_st | 2.23964492 | 2.61984501 | 2.256999951 | 2.805858484 | 1.802330574 | 1.941506129 | 1.95410658 |
| hsa-miR-1243_st | 2.990535773 | 2.92899382 | 2.661690595 | 3.618980988 | 2.227277173 | 3.012109232 | 3.108349089 |
| hsa-miR-548 [symbol] _st | 0.794552682 | 0.697855346 | 1.159502729 | 0.834966099 | 0.303356803 | 0.943249207 | 0.483959665 |
| hsa-miR-1244_st | 8.071397053 | 8.149003012 | 9.050160098 | 8.981644705 | 8.914975273 | 8.995573895 | 8.118169322 |
| hsa-miR-1245_st | 2.828472282 | 3.326473254 | 2.79538873 | 3.004523028 | 3.278243796 | 3.603673474 | 3.319459402 |
| hsa-miR-1246_st | 13.02643745 | 13.26400421 | 13.13752855 | 13.2191444 | 12.78620332 | 12.85934439 | 13.07470331 |
| hsa-miR-1247_st | 6.597744835 | 5.663792009 | 6.239961809 | 5.746350172 | 5.952444283 | 7.664384714 | 6.013757726 |
| hsa-miR-1248_st | 9.313928653 | 9.664122204 | 9.971933553 | 10.62740335 | 10.32738997 | 9.725973627 | 10.1256414 |
| hsa-miR-1249_st | 4.971083895 | 4.352905935 | 4.684337858 | 4.88883234 | 5.657470183 | 5.147616145 | 4.619079982 |
| hsa-miR-1250_st | 8.67918742 | 7.737641888 | 8.1873687 | 8.225551512 | 9.30282666 | 8.307473653 | 9.080009271 |
| hsa-miR-1251_st | 3.718263732 | 4.621119252 | 5.715141443 | 6.979903888 | 3.941686766 | 3.336985741 | 5.182304921 |
| hsa-miR-1253_st | 4.269887958 | 3.806724699 | 4.531717736 | 4.619131876 | 4.432304947 | 4.186553905 | 4.256422889 |
| hsa-miR-1254_st | 11.23587328 | 10.50683709 | 10.24874425 | 10.60287101 | 11.0219449 | 11.98363338 | 11.23120595 |
| hsa-miR-1255a_st | 5.365524479 | 4.672347076 | 5.159273303 | 5.569252323 | 5.555665295 | 4.577440684 | 4.625339589 |
| hsa-miR-1256_st | 3.199576116 | 3.17428179 | 4.70136052 | 3.983161789 | 4.342417656 | 2.008958653 | 1.531976485 |
| hsa-miR-1257_st | 3.54633271 | 3.653829094 | 4.132731258 | 3.427909302 | 4.352579394 | 3.552421552 | 4.346040535 |
| hsa-miR-1258_st | 3.946593477 | 4.098563446 | 3.1897803 | 3.183865086 | 5.808573884 | 4.041570964 | 4.564685123 |
| hsa-miR-1259_st | 1.608780695 | 2.565086439 | 2.366787991 | 2.84370509 | 1.985231452 | 1.401969999 | 1.202509585 |
| hsa-miR-1260_st | 10.40275691 | 11.08287471 | 10.50666654 | 10.51230143 | 9.589388795 | 10.0806496 | 10.3308085 |
| hsa-miR-548g_st | 0.581732821 | 1.380152389 | 0.561799773 | −0.018105876 | −0.7912175 | 0.437944333 | 0.454037462 |
| hsa-miR-1261_st | 7.170204544 | 6.750779416 | 6.86075105 | 7.417506659 | 8.046426671 | 8.217420936 | 8.40684468 |
| hsa-miR-1262_st | 1.006115209 | 2.511087897 | 2.432251778 | 1.729033225 | 2.632286527 | 1.000295989 | 2.490891676 |
| hsa-miR-1263_st | 3.671072331 | 2.43258445 | 3.041120001 | 2.828042706 | 3.441202234 | 3.223552053 | 3.789065134 |
| hsa-miR-548n_st | 1.492210457 | 1.334915506 | 2.630167506 | 1.328713453 | 1.392540297 | 1.69160298 | 0.139778105 |
| hsa-miR-548m_st | 2.220539483 | 2.403331786 | 2.914787083 | 3.621147345 | 2.90030524 | 1.076870008 | 1.223740093 |
| hsa-miR-1265_st | 4.879670511 | 4.960251428 | 5.128342147 | 4.607699911 | 5.366557894 | 3.589632418 | 4.12516532 |
| hsa-miR-548o_st | 2.550235755 | 1.809516108 | 1.036974417 | 1.40203856 | 2.401846175 | 0.246171242 | 0.886316106 |
| hsa-miR-1266_st | 8.092477746 | 7.619307137 | 7.834546953 | 8.404740079 | 8.485189511 | 9.388296014 | 8.62435427 |
| hsa-miR-1267_st | 4.265216641 | 3.343352841 | 3.658090892 | 2.785731758 | 3.235208875 | 3.853123412 | 3.680739074 |
| hsa-miR-1268_st | 14.94917819 | 15.2219134 | 14.44293943 | 14.86718959 | 14.36722631 | 15.67421174 | 15.29265589 |
| hsa-miR-1269_st | 4.428153478 | 4.870550306 | 4.718878363 | 4.029209915 | 5.309937431 | 5.059762216 | 5.058944544 |
| hsa-miR-1270_st | 5.740805301 | 6.199652161 | 5.532897024 | 6.79685106 | 6.373376601 | 6.279767358 | 6.051949953 |
| hsa-miR-1272_st | 3.467358832 | 3.223719021 | 3.892802021 | 2.069955945 | 4.318773544 | 2.835849596 | 3.27362261 |
| hsa-miR-1273_st | 11.03257812 | 11.09945265 | 10.60483481 | 10.45707144 | 11.18984734 | 10.30967973 | 10.95998332 |
| hsa-miR-1274a_st | 10.63013359 | 11.05078298 | 10.67598948 | 10.88263655 | 10.19071206 | 11.49131218 | 11.00006558 |
| hsa-miR-548h_st | 4.074263999 | 3.256563591 | 4.375861485 | 2.998969925 | 2.35328987 | 2.024109919 | 2.359912074 |
| hsa-miR-1275_st | 13.81351143 | 13.3993736 | 13.29753808 | 13.55323555 | 13.74736678 | 14.28979841 | 14.19099142 |
| hsa-miR-1276_st | 5.864459249 | 5.698057284 | 5.304785316 | 4.532261763 | 6.449252439 | 6.007186759 | 6.279568717 |
| hsa-miR-302 [symbol] _st | 2.82401543 | 2.097613458 | 3.111637824 | 2.791077509 | 4.166208469 | 1.086448182 | 2.500091196 |
| hsa-miR-302 [symbol] _st | 0.966171712 | 1.875028865 | 1.44963233 | 1.514685554 | 0.603706852 | 1.490830469 | 0.82029764 |
| hsa-miR-1277_st | 1.457847327 | 2.017360794 | 2.266847891 | 1.952479553 | 1.510541575 | 1.404568286 | 1.665504545 |
| hsa-miR-548p_st | 1.44039033 | 1.678206956 | 0.866109068 | 1.429777948 | 0.656395673 | 2.134872838 | 1.145599335 |
| hsa-miR-548i_st | 5.489830277 | 4.41608705 | 5.403790274 | 4.904343926 | 5.327893788 | 3.220426363 | 3.484818987 |
| hsa-miR-1278_st | 2.959075373 | 3.665362256 | 3.778179236 | 4.424875182 | 2.49945755 | 2.888658728 | 2.843858005 |
| hsa-miR-1279_st | 3.011420483 | 2.980631994 | 4.86563415 | 3.857222067 | 4.241671924 | 1.414593802 | 1.881452527 |
| hsa-miR-1274b_st | 13.8953473 | 14.0830953 | 14.24051857 | 14.12473728 | 13.24421404 | 14.25372017 | 13.91959034 |
| hsa-miR-1281_st | 9.066540175 | 8.396412021 | 8.205188893 | 8.345758838 | 9.021066245 | 9.0653027 | 9.296346678 |
| hsa-miR-1282_st | 4.632387273 | 3.804729646 | 4.832931552 | 4.50046572 | 5.152020072 | 2.786353282 | 3.666140393 |
| hsa-miR-1284_st | 4.439892501 | 3.745898523 | 3.748503642 | 4.156305087 | 4.868053547 | 3.852632084 | 3.327636599 |
| hsa-miR-1288_st | 4.250362802 | 4.732828893 | 3.712746617 | 4.773842697 | 4.055263386 | 3.981287333 | 4.47175624 |
| hsa-miR-1292_st | 12.0799599 | 11.80351024 | 11.60304513 | 11.748145 | 12.11399642 | 13.15674427 | 12.70349083 |
| hsa-miR-1252_st | 1.631417262 | 1.436528832 | 1.488617827 | 2.070940555 | 1.098821961 | 1.988367223 | 2.057676929 |
| hsa-miR-1255b_st | 8.882065385 | 8.397153807 | 8.49154682 | 7.498980109 | 8.60803343 | 8.024935844 | 7.708445133 |
| hsa-miR-1280_st | 14.80588843 | 15.40611039 | 14.83091836 | 14.90898491 | 13.71965271 | 14.9361986 | 14.74120197 |
| hsa-miR-1308_st | 14.93604122 | 15.42784598 | 14.63544424 | 14.84622385 | 14.37932811 | 15.56819828 | 15.27865834 |
| hsa-miR-664-star_st | 10.08824902 | 9.116293351 | 10.23776913 | 9.220519912 | 9.826454758 | 9.259838538 | 9.92416625 |
| hsa-miR-664_st | 5.436691335 | 4.746876098 | 5.091850407 | 5.459585746 | 5.702010646 | 5.665704983 | 5.944617488 |
| hsa-miR-1306_st | 5.952731712 | 6.308482501 | 6.432773142 | 6.542987273 | 7.507046709 | 6.872932356 | 6.765366092 |
| hsa-miR-1307_st | 13.30535552 | 13.38702044 | 12.50833195 | 13.24395914 | 12.99216439 | 14.28752436 | 13.91560532 |
| hsa-miR-513b_st | 9.667055463 | 9.613555404 | 10.70872942 | 10.6330214 | 10.86092251 | 10.97497465 | 11.21328094 |
| hsa-miR-513c_st | 8.297081404 | 7.536278229 | 8.22461603 | 8.104812489 | 9.251092175 | 9.092323849 | 9.296934924 |
| hsa-miR-1321_st | 6.901093894 | 6.771097702 | 7.106131501 | 6.930425872 | 7.640096684 | 6.908974632 | 7.761179472 |
| hsa-miR-1322_st | 3.914019806 | 3.448498164 | 3.470609506 | 3.540354824 | 4.24332251 | 3.692631473 | 3.362141243 |
| hsa-miR-720_st | 14.98882581 | 15.37395921 | 15.09005902 | 15.01040651 | 13.91270298 | 15.43901846 | 14.54973903 |
| hsa-miR-1197_st | 2.01805118 | 2.623844912 | 2.347619833 | 1.961806181 | 1.783893079 | 0.888667431 | 1.842047585 |
| hsa-miR-1324_st | 1.607923354 | 1.947692293 | 2.354792636 | 1.112990681 | 1.643093598 | 1.455884152 | 2.213429778 |
| hsa-miR-1469_st | 15.22828253 | 15.86860022 | 15.16955717 | 15.44886069 | 14.53291464 | 15.83157801 | 15.68485596 |
| hsa-miR-1470_st | 5.585456142 | 4.922763035 | 4.388830314 | 5.365528782 | 5.588502574 | 6.274821586 | 5.155740393 |
| hsa-miR-1471_st | 10.88796331 | 10.12701766 | 9.854973102 | 9.905605182 | 11.03480385 | 11.21767568 | 11.5380136 |
| hsa-miR-1537_st | 1.595276342 | 2.037066372 | 1.71436831 | 0.814158105 | 0.588147159 | 0.673061764 | 1.179419767 |
| hsa-miR-1538_st | 7.268420994 | 7.615397332 | 7.108693022 | 6.999986308 | 8.058420376 | 8.386394029 | 7.605800874 |
| hsa-miR-1539_st | 5.916922553 | 5.990814163 | 5.679635753 | 5.834709318 | 6.014130129 | 6.241943669 | 6.123561058 |
| hsa-miR-103-as_st | 2.752161768 | 2.291901794 | 2.139348341 | 2.810900456 | 3.813760476 | 1.814992319 | 1.621332063 |
| hsa-miR-320d_st | 9.406603759 | 9.800391485 | 10.54924815 | 10.0589528 | 8.816054245 | 9.250202494 | 9.416120982 |
| hsa-miR-1825_st | 6.190463203 | 6.018731663 | 6.132994182 | 6.524292016 | 7.055299397 | 7.062129851 | 6.442960872 |
| hsa-miR-1826_st | 15.50587766 | 16.26207727 | 15.16633314 | 15.49885302 | 14.5339472 | 15.83083235 | 15.71017665 |
| hsa-miR-1827_st | 3.843786348 | 5.639758447 | 6.578011481 | 6.532564432 | 6.018656798 | 4.15497128 | 5.465714373 |
| hsa-miR-1908_st | 15.36672371 | 16.12617118 | 15.30266162 | 15.50010419 | 14.53389508 | 15.83102386 | 15.71031479 |
| hsa-miR-1909-star_st | 8.862390484 | 8.156753439 | 7.975106092 | 8.265969146 | 8.537712758 | 8.99077341 | 9.129826616 |
| hsa-miR-1909_st | 14.40397812 | 14.25268364 | 13.81198373 | 14.16695026 | 14.16764201 | 14.91311077 | 14.73535624 |
| hsa-miR-1910_st | 10.20115232 | 8.660954391 | 8.936665359 | 9.215033298 | 9.169677685 | 10.00585819 | 9.738615629 |
| hsa-miR-1911_st | 3.470392012 | 3.423764108 | 3.422255662 | 3.058182882 | 4.74411524 | 3.696934131 | 3.278108035 |
| hsa-miR-1911-star_st | 4.827063796 | 5.008613494 | 4.891899693 | 6.00476189 | 4.507083219 | 4.636583617 | 4.818668204 |
| hsa-miR-1912_st | 3.589225811 | 3.815706193 | 4.576248829 | 4.089330481 | 3.877241564 | 3.922984142 | 3.667678247 |
| hsa-miR-1913_st | 8.075766292 | 7.562275017 | 7.198735568 | 7.278278815 | 7.959859314 | 8.76903596 | 8.517550318 |
| hsa-miR-1914_st | 7.061996312 | 6.279432211 | 7.077486342 | 6.732634269 | 7.131241378 | 6.77243328 | 6.321844635 |
| hsa-miR-1914-star_st | 12.36812436 | 12.08851744 | 11.79509276 | 12.25285398 | 11.92912213 | 12.90122417 | 13.03353095 |
| hsa-miR-1915-star_st | 7.824000995 | 7.452228103 | 7.577943591 | 7.17808095 | 8.099955499 | 7.066602395 | 6.8620742 |
| hsa-miR-1915_st | 15.38161198 | 16.11295899 | 15.26682994 | 15.4991243 | 14.53352223 | 15.83184979 | 15.70979606 |
| hsa-miR-1972_st | 13.82967707 | 13.47030929 | 13.10664147 | 12.63883731 | 13.18332823 | 12.93487135 | 12.48483901 |
| hsa-miR-1973_st | 13.97074855 | 14.67243449 | 14.31288182 | 14.27979701 | 14.5350626 | 15.20462859 | 15.24363118 |
| hsa-miR-1975_st | 15.4931759 | 16.02278069 | 15.30512044 | 15.49540235 | 14.52992145 | 15.77362369 | 15.59277397 |
| hsa-miR-1976_st | 7.931872509 | 7.110831669 | 7.286069909 | 6.903276481 | 7.842389157 | 7.735508039 | 7.344587623 |
| hsa-miR-1979_st | 15.06256507 | 15.30524163 | 14.40346944 | 15.23733244 | 14.17434994 | 14.86590562 | 15.11579688 |
| hsa-miR-2052_st | −0.18128035 | 1.307712753 | 0.673732632 | −0.066622305 | 0.171310892 | 1.579574611 | 0.24651085 |
| hsa-miR-2053_st | 0.306674829 | 2.256648569 | 1.590859985 | 2.698192286 | 2.25588578 | 0.838845201 | 1.090029472 |
| hsa-miR-2054_st | −0.377192814 | 0.815436126 | −0.802650027 | −0.458920601 | −0.878376175 | 0.225891486 | −0.685179269 |
| hsa-miR-2110_st | 10.28769483 | 10.03467203 | 9.829702772 | 10.33710626 | 10.16696434 | 10.95661784 | 11.15048165 |
| hsa-miR-2114_st | 2.46894214 | 2.04622731 | 2.733136858 | 2.646066249 | 3.59475051 | 13.519096866 | 2.357837486 |
| hsa-miR-2114-star_st | 6.413481302 | 5.630244799 | 5.650849443 | 4.730436828 | 7.961763628 | 7.032048356 | 6.455600705 |
| hsa-miR-2115_st | 1.238749796 | 2.381950617 | 2.03651834 | 2.34454499 | 2.111704986 | 1.485596539 | 1.504416397 |
| hsa-miR-2115-star_st | 7.920738142 | 7.683484511 | 6.421142555 | 7.269441523 | 9.250687785 | 7.824862466 | 7.504259856 |
| hsa-miR-2116_st | 6.307916912 | 4.830066678 | 5.286780453 | 4.386678636 | 5.800974647 | 6.236758362 | 5.611345917 |
| hsa-miR-2116-star_st | 4.840520647 | 4.953969915 | 4.325339187 | 4.861397804 | 5.449105487 | 3.901983075 | 4.071903599 |
| hsa-miR-2117_st | 1.705858466 | 1.685713595 | 1.739933999 | 0.79367315 | 3.620336274 | 2.388775658 | 1.025898666 |
| hsa-miR-548q_st | 7.433378006 | 7.363851764 | 6.702843358 | 6.471464736 | 7.876758315 | 7.789902386 | 6.320124994 |
| hsa-miR-2276_st | 9.776282102 | 9.289609553 | 9.254087226 | 9.477493969 | 9.61247373 | 10.38477688 | 10.13088508 |
| hsa-miR-2277_st | 8.206473651 | 8.144681781 | 7.403499965 | 8.058947607 | 8.548308566 | 8.971237002 | 8.850862671 |
| hsa-miR-2278_st | 5.254669392 | 5.091371732 | 5.024829664 | 5.362573062 | 5.299503812 | 5.590651707 | 4.866894469 |
| hsa-miR-711_st | 10.908872 | 10.22535162 | 10.66560957 | 11.02474765 | 11.35633374 | 11.88371834 | 11.88903123 |
| hsa-miR-718_st | 7.772221731 | 6.40575076 | 6.954535025 | 7.000859731 | 7.523553932 | 8.494278162 | 7.624311889 |
| hsa-miR-2861_st | 15.36791735 | 16.16258847 | 15.29234121 | 15.49875229 | 14.53406393 | 15.83133194 | 15.71056029 |
| hsa-miR-2909_st | 2.545817726 | 3.09793504 | 3.319480833 | 3.137747413 | 3.322000679 | 2.935254682 | 2.458677095 |
| hsa-miR-3115_st | 1.413278401 | 1.936272822 | 2.243885261 | 0.285815826 | 2.467287976 | 1.701047637 | 2.16057981 |
| hsa-miR-3116_st | 5.677384586 | 5.726605124 | 5.741660793 | 5.769077555 | 6.433749108 | 6.699159002 | 6.04880894 |
| hsa-miR-3117_st | 2.485330186 | 2.789571714 | 3.015089228 | 1.912330942 | 3.109854617 | 2.5515233 | 2.91833621 |
| hsa-miR-3118_st | 1.572829689 | 2.55875517 | 3.063745626 | 2.331618144 | 1.721606823 | 1.509211296 | 2.203582079 |
| hsa-miR-3119_st | 1.41954092 | 1.234176916 | 0.910753926 | 1.876234397 | 0.574332187 | 1.252412629 | −0.276649999 |
| hsa-miR-3120_st | 5.751863007 | 5.248937981 | 5.334837406 | 5.667002805 | 5.993567011 | 5.610385696 | 6.04066769 |
| hsa-miR-3121_st | 6.382228955 | 6.334919499 | 6.253458159 | 6.166158122 | 7.149443668 | 6.829141554 | 7.384561764 |
| hsa-miR-3122_st | 10.56094112 | 10.24325543 | 9.814864983 | 10.78809293 | 10.7331607 | 11.61415433 | 11.34042164 |
| hsa-miR-3123_st | 0.630043246 | 0.062765885 | −0.372385274 | 0.634308373 | −0.839670623 | −0.424664146 | −0.352527953 |
| hsa-miR-3124_st | 11.52662958 | 11.10808422 | 10.38498517 | 10.95239681 | 11.16450362 | 11.83724151 | 11.85885934 |
| hsa-miR-548s_st | 1.865987636 | 1.854746135 | 1.765217405 | 1.59232995 | 1.626525126 | 1.452279379 | 1.549474029 |
| hsa-miR-3125_st | 6.274721597 | 6.604860727 | 6.520971426 | 7.370989873 | 7.180788497 | 7.145909731 | 6.839644617 |
| hsa-miR-3126-5p_st | 11.3355542 | 10.58798571 | 11.03759911 | 11.21402991 | 11.60028585 | 12.07346528 | 12.2521444 |
| hsa-miR-3126-3p_st | 5.012886706 | 3.939803177 | 4.275618002 | 4.765615023 | 4.424292168 | 4.292269032 | 4.834780084 |
| hsa-miR-3127_st | 8.917610582 | 8.606263832 | 8.460299648 | 9.048219178 | 8.965362233 | 9.304704128 | 9.484717375 |
| hsa-miR-3128_st | 2.680030316 | 2.758055721 | 2.57417768 | 1.845215941 | 3.155247851 | 2.465893166 | 2.360842349 |
| hsa-miR-3129_st | 3.615105572 | 3.132488677 | 3.510828592 | 3.561733342 | 4.149439046 | 3.24320416 | 3.302787248 |
| hsa-miR-3130-5p_st | 6.449619782 | 5.988501074 | 5.98227786 | 6.433692886 | 7.236972148 | 6.572643261 | 7.009255958 |
| hsa-miR-3130-3p_st | 7.461910758 | 6.98517947 | 6.35618028 | 7.320098766 | 7.480741766 | 8.155804133 | 7.545450659 |
| hsa-miR-3131_st | 11.7162425 | 10.9505356 | 11.16040852 | 11.03121604 | 11.65406796 | 12.19364465 | 12.13119762 |
| hsa-miR-3132_st | 7.929125545 | 8.126921693 | 8.282559672 | 8.790891277 | 8.478483894 | 9.192818924 | 9.14546933 |
| hsa-miR-3133_st | 2.28574953 | 2.583856224 | 2.931582644 | 2.988642277 | 2.492678904 | 2.110154416 | 3.388543432 |
| hsa-miR-378b_st | 8.190594212 | 8.727377863 | 8.146824383 | 8.464604673 | 8.847551957 | 8.96409278 | 8.860412442 |
| hsa-miR-3134_st | 1.949163642 | 2.48171606 | 1.336308084 | 1.293095066 | 2.412839883 | 0.324418566 | 0.931937896 |
| hsa-miR-3135_st | 6.666898454 | 5.60813541 | 5.832960002 | 5.770409101 | 6.324432599 | 6.577521852 | 6.051698139 |
| hsa-miR-466_st | 6.256922382 | 5.752867775 | 5.39606828 | 5.390063769 | 6.992065298 | 4.706598296 | 5.476033225 |
| hsa-miR-3136_st | 5.964058112 | 5.613858668 | 5.30212754 | 5.617211545 | 6.692776019 | 5.987710392 | 6.685015217 |
| hsa-miR-544b_st | 1.961536427 | 2.289792259 | 2.57099512 | 2.443863245 | 3.086661191 | 1.793993462 | 2.165405534 |
| hsa-miR-3137_st | 10.20625676 | 9.918962387 | 9.526890276 | 10.40964899 | 10.16166641 | 11.23855577 | 10.78316545 |
| hsa-miR-3138_st | 10.32080424 | 9.488447865 | 10.19384462 | 10.34025937 | 10.16832272 | 11.26928833 | 11.31472514 |
| hsa-miR-3139_st | 2.439885547 | 2.414646654 | 2.226334709 | 2.006461653 | 2.801687578 | 2.411887039 | 1.985186232 |
| hsa-miR-3140_st | 4.834043967 | 4.639718588 | 3.740248916 | 3.572295201 | 4.688831015 | 4.665224443 | 4.624949544 |
| hsa-miR-548t_st | 1.747013982 | 2.76990208 | 2.31734993 | 3.2316209 | 2.575190622 | 2.292165516 | 2.549902427 |
| hsa-miR-3141_st | 14.56932438 | 14.7291538 | 14.14800755 | 14.49120012 | 14.16930883 | 15.37479435 | 15.09188915 |
| hsa-miR-3142_st | 1.695312345 | 1.469072187 | 1.059172518 | 1.166858182 | 1.26804272 | 1.643860314 | 1.731127528 |
| hsa-miR-3143_st | 0.07966077 | 1.11802397 | 0.638403259 | −0.138626943 | 1.362845873 | −0.05359338 | −0.106609177 |
| hsa-miR-548u_st | 1.651926922 | 1.59539641 | 2.421179281 | 2.367234176 | 2.925022775 | 1.637060588 | 0.615487311 |
| hsa-miR-3144-5p_st | 4.882875369 | 4.656077992 | 4.644484301 | 5.118924567 | 5.672945435 | 4.968066575 | 4.916071726 |
| hsa-miR-3144-3p_st | 2.738952499 | 3.000865689 | 2.533654533 | 2.747709897 | 3.17254822 | 2.617009905 | 3.990977388 |
| hsa-miR-3145_st | 0.055473026 | 0.67763553 | 0.188116774 | 0.049766145 | 0.779210549 | 0.889256046 | 0.286714613 |
| hsa-miR-1273c_st | 9.90472503 | 9.43537457 | 9.795252743 | 9.970191241 | 10.09048653 | 10.17754341 | 10.42008806 |
| hsa-miR-3146_st | 4.20240964 | 3.705415929 | 3.587421991 | 2.35180705 | 3.803379498 | 3.158513046 | 3.061553252 |
| hsa-miR-3147_st | 11.13768833 | 10.62937171 | 10.60001109 | 10.72587768 | 10.82724064 | 11.37846378 | 11.27473106 |
| hsa-miR-548v_st | 2.442368027 | 2.988389393 | 3.707428018 | 3.527207974 | 3.641251783 | 1.321764709 | 3.457068611 |
| hsa-miR-3148_st | 5.198093012 | 4.42720859 | 3.875386632 | 4.762473421 | 5.227989568 | 5.008262754 | 4.108404148 |
| hsa-miR-3149_st | 2.809794529 | 1.940989406 | 4.057900151 | 4.149343158 | 3.475687514 | 2.274564816 | 2.093383297 |
| hsa-miR-3150_st | 8.802378504 | 8.434243262 | 8.28177163 | 9.130804562 | 9.103377404 | 9.623332485 | 9.4401968 |
| hsa-miR-3151_st | 10.79586281 | 10.52252658 | 10.33300954 | 10.64251968 | 10.48394041 | 11.26211321 | 11.15687361 |
| hsa-miR-3152_st | 6.477694999 | 6.390645607 | 6.373901124 | 6.299514522 | 6.759208573 | 6.989595331 | 6.521175326 |
| hsa-miR-3153_st | 9.120933155 | 8.874850694 | 8.459952297 | 8.817173222 | 9.000091796 | 9.120393189 | 9.529892758 |
| hsa-miR-3074_st | 5.119070445 | 4.140910206 | 4.288173995 | 4.125670093 | 4.087623133 | 3.979735238 | 3.744019594 |
| hsa-miR-3154_st | 11.32335921 | 10.71790071 | 10.63956328 | 10.89604519 | 10.95482735 | 11.79220463 | 11.57509418 |
| hsa-miR-3155_st | 6.530807823 | 6.317042053 | 6.53503041 | 6.468660911 | 7.316242338 | 7.337678303 | 7.002524519 |
| hsa-miR-3156_st | 10.05317266 | 9.909538439 | 9.959304197 | 10.29802435 | 9.773439985 | 10.49110833 | 10.28648875 |
| hsa-miR-3157_st | 5.32255978 | 4.829436489 | 4.885936538 | 4.87600744 | 5.357329584 | 5.559204422 | 5.501835857 |
| hsa-miR-3158_st | 3.695240213 | 4.229479521 | 4.187219188 | 3.880908243 | 5.194370948 | 4.414033841 | 4.002877263 |
| hsa-miR-3159_st | 3.34413761 | 3.478509635 | 4.152360558 | 3.235383658 | 3.686109282 | 4.39526905 | 3.740007101 |
| hsa-miR-3160_st | 5.055148 | 5.314675646 | 5.097657276 | 5.229171559 | 5.68510048 | 5.851492984 | 5.282940917 |
| hsa-miR-3161_st | 6.47670426 | 6.347623956 | 5.888156391 | 5.97226241 | 7.539654786 | 7.092479234 | 7.257155693 |
| hsa-miR-3162_st | 13.11527046 | 12.89974007 | 12.44563495 | 13.26225432 | 12.99085333 | 14.04745034 | 13.7503546 |
| hsa-miR-3163_st | 7.13001139 | 7.294146427 | 7.073398267 | 8.103671973 | 7.949557354 | 8.076270978 | 7.208437079 |
| hsa-miR-3164_st | 6.12066392 | 6.180401533 | 5.689981304 | 6.070208195 | 6.706305381 | 6.374133679 | 6.537639188 |
| hsa-miR-3165_st | 5.93187898 | 5.980026721 | 6.205619096 | 7.173720586 | 6.626518007 | 7.03737729 | 7.412012516 |
| hsa-miR-3166_st | 4.019428406 | 4.33335431 | 3.824869502 | 4.008568204 | 3.861944297 | 4.529972953 | 3.728886515 |
| hsa-miR-1260b_st | 13.80865982 | 14.5533218 | 13.73049611 | 14.34257205 | 12.84707752 | 13.69539862 | 13.86175647 |
| hsa-miR-3167_st | 1.130398286 | 2.57433395 | 0.204145248 | 0.263799113 | 0.809023023 | 1.284475072 | 1.753026482 |
| hsa-miR-3168_st | 1.773244888 | 2.531243657 | 2.264887253 | 0.912632519 | 2.73540565 | 1.682399797 | 2.007728212 |
| hsa-miR-3169_st | 3.932399473 | 3.367649915 | 3.450176152 | 4.043607136 | 4.034873855 | 3.564478119 | 4.587484528 |
| hsa-miR-3170_st | 5.773286425 | 5.530039056 | 5.61107952 | 5.779599049 | 6.390023354 | 6.455677044 | 5.868933465 |
| hsa-miR-3171_st | 2.080327009 | 2.546224501 | 2.6166347 | 1.916591221 | 0.778886534 | 1.955187015 | 0.646801465 |
| hsa-miR-3172_st | 14.87003476 | 15.64546516 | 14.72631397 | 14.87603789 | 14.30953143 | 14.75071956 | 14.94756248 |
| hsa-miR-3173_st | 8.473346789 | 8.170716112 | 8.596609895 | 8.858337274 | 9.017505288 | 9.4327819 | 9.38343232 |
| hsa-miR-1193_st | 9.370720477 | 9.256452167 | 9.600637791 | 9.835350028 | 9.773822736 | 10.28727306 | 10.3316528 |
| hsa-miR-323b-5p_st | 8.046981225 | 7.1640292 | 6.925509259 | 7.016500356 | 7.600071672 | 7.463741081 | 7.300280224 |
| hsa-miR-323b-3p_st | 2.782520587 | 3.074113806 | 2.753225566 | 3.25297221 | 2.655059942 | 2.330341776 | 3.096981124 |
| hsa-miR-3174_st | 6.390068347 | 5.710256118 | 6.258946335 | 6.526312048 | 7.336500546 | 6.409653183 | 5.944397141 |
| hsa-miR-3175_st | 12.86018919 | 12.68191769 | 12.71895312 | 12.63019441 | 12.67627018 | 12.94779946 | 13.20819847 |
| hsa-miR-3176_st | 10.71752041 | 10.72694394 | 10.1458847 | 10.44005268 | 10.60051773 | 11.19572837 | 10.76012955 |
| hsa-miR-3177_st | 11.22322405 | 10.67969762 | 10.49638801 | 11.03673274 | 11.02697344 | 12.0487339 | 11.86200914 |
| hsa-miR-3178_st | 14.84047163 | 15.31685611 | 14.92355508 | 15.04914233 | 14.5322918 | 15.73859723 | 15.51317343 |
| hsa-miR-3179_st | 3.108401656 | 2.560647565 | 3.108999627 | 2.18932308 | 4.97078861 | 3.054140159 | 2.745499944 |
| hsa-miR-3180-5p_st | 7.763786493 | 6.662718492 | 6.795519097 | 7.267921181 | 7.147126368 | 7.942463868 | 7.372625186 |
| hsa-miR-3180-3p_st | 14.23895306 | 14.09768183 | 13.39441555 | 13.90923056 | 13.8212402 | 14.86180169 | 14.56874938 |
| hsa-miR-548w_st | 7.089096798 | 6.028725816 | 7.075623596 | 6.349187431 | 7.048076456 | 4.761813269 | 4.865359559 |
| hsa-miR-3181_st | 8.39148411 | 7.792795003 | 7.091914496 | 7.128721022 | 7.607516563 | 9.453165835 | 8.45296633 |
| hsa-miR-3182_st | 2.36896044 | 2.959051607 | 4.134028873 | 3.165686076 | 2.933380323 | 1.733975959 | 2.958692548 |
| hsa-miR-3183_st | 3.290039111 | 3.738264903 | 2.888159021 | 4.231102076 | 4.042987908 | 4.480911075 | 3.744895152 |
| hsa-miR-3184_st | 9.228275122 | 8.813382265 | 9.020348021 | 8.764044648 | 9.386267473 | 9.783788117 | 9.305150581 |
| hsa-miR-3185_st | 14.57875923 | 14.73526052 | 13.55456919 | 14.45158868 | 14.1436258 | 15.33260865 | 15.03765902 |
| hsa-miR-3065-5p_st | 1.912887773 | 4.445529232 | 5.287276829 | 4.293033284 | 3.376885305 | 2.685381418 | 4.110415907 |
| hsa-miR-3065-3p_st | 4.413279466 | 4.965085074 | 4.601754955 | 3.838988301 | 4.285595821 | 4.088492137 | 5.215985707 |
| hsa-miR-3186-5p_st | 4.460190355 | 4.286739391 | 4.3378327 | 3.324214963 | 5.275866057 | 3.900751303 | 3.658559316 |
| hsa-miR-3186-3p_st | 8.539669572 | 8.12784033 | 8.067931706 | 8.285220828 | 8.504953111 | 8.891588008 | 8.713370275 |
| hsa-miR-3187_st | 12.17035214 | 11.6282248 | 11.29841344 | 11.80226783 | 12.54423143 | 12.85471945 | 12.55441583 |
| hsa-miR-3188_st | 12.48690106 | 12.20877012 | 11.41013801 | 12.31535728 | 12.11279358 | 13.51555744 | 13.13152604 |
| hsa-miR-3189_st | 6.886016536 | 6.809562929 | 6.436718463 | 6.769883632 | 7.175720634 | 7.892821596 | 7.148431141 |
| hsa-miR-320 [symbol] _st | 9.263855681 | 9.476331059 | 10.0868345 | 9.786200932 | 8.620602766 | 9.123092563 | 9.203596286 |
| hsa-miR-3190-5p_st | 9.141092614 | 9.016443616 | 9.159709204 | 9.56645797 | 9.802986052 | 10.45564545 | 10.16608014 |
| hsa-miR-3190-3p_st | 9.462605136 | 9.334991533 | 9.358244185 | 9.750910969 | 9.656566043 | 10.6908063 | 10.35295762 |
| hsa-miR-3191_st | 11.52255255 | 10.61495389 | 10.99027282 | 11.34740057 | 11.43982861 | 11.82071177 | 11.94331995 |
| hsa-miR-3192_st | 8.649371986 | 8.126272303 | 7.991371006 | 8.496685235 | 8.954839124 | 8.961641632 | 9.006631762 |
| hsa-miR-3193_st | 5.679530464 | 5.506540092 | 6.127073005 | 5.804503529 | 6.14028567 | 6.071393175 | 6.45871691 |
| hsa-miR-3194_st | 10.15681871 | 9.540791858 | 9.579243042 | 9.874483237 | 10.37106851 | 10.79481532 | 10.78915705 |
| hsa-miR-3195_st | 12.70965904 | 12.16703876 | 11.54220032 | 11.79419203 | 12.21212559 | 13.02887324 | 12.86349721 |
| hsa-miR-3196_st | 15.53905776 | 16.28170265 | 15.31135483 | 15.49927968 | 14.53181774 | 15.83137173 | 15.71070786 |
| hsa-miR-548x_st | 0.639325962 | 0.901282077 | 0.79929523 | 0.628391667 | 0.129471688 | 0.204108023 | −0.19213261 |
| hsa-miR-3197_st | 14.18362318 | 14.65065527 | 14.4340179 | 14.69235717 | 14.5005803 | 15.35014127 | 15.19538457 |
| hsa-miR-3198_st | 8.909009189 | 8.608614477 | 8.625271539 | 9.145051516 | 9.16587878 | 10.11049917 | 9.589649085 |
| hsa-miR-3199_st | 5.263134701 | 4.38074435 | 4.364344803 | 4.320157187 | 5.704452414 | 4.358384096 | 5.416730415 |
| hsa-miR-3200_st | 3.161033776 | 4.025575105 | 4.692256084 | 4.673284515 | 3.624632287 | 2.06738048 | 4.135898179 |
| hsa-miR-3201_st | 3.541248819 | 3.805494447 | 3.063865135 | 3.38511774 | 2.768449591 | 3.319162743 | 3.889435684 |
| hsa-miR-514b-5p_st | 9.434179442 | 9.153702323 | 9.217463565 | 9.578007798 | 9.967058538 | 10.06856653 | 9.776149666 |
| hsa-miR-514b-3p_st | 1.976316805 | 1.989856394 | 1.426155483 | 1.435348716 | 1.742027815 | 2.411813925 | 1.206783685 |
| hsa-miR-3202_st | 7.073189993 | 7.112850402 | 7.443557073 | 7.568616773 | 7.898026042 | 7.942985935 | 7.861975679 |
| hsa-miR-1273d_st | 12.07836091 | 11.66695293 | 11.49633373 | 11.18967281 | 11.12850142 | 11.02290196 | 11.00169041 |
| hsa-miR-4295_st | 2.219466857 | 2.139421816 | 2.805674139 | 1.254592781 | 3.023111323 | 1.315861446 | 2.068824506 |
| hsa-miR-4296_st | 7.48491885 | 6.90760476 | 6.108965523 | 5.35510301 | 7.7512865 | 6.754467285 | 6.81347504 |
| hsa-miR-4297_st | 5.670322987 | 4.98980718 | 5.650017627 | 5.439531413 | 6.497076537 | 4.966878864 | 6.07761014 |
| hsa-miR-378c_st | 8.075101699 | 8.033645257 | 8.808352625 | 9.100213596 | 8.746313946 | 9.122998121 | 8.330768291 |
| hsa-miR-4293_st | 4.37172883 | 4.109828109 | 4.086080757 | 4.307026963 | 4.275520746 | 4.547283907 | 5.392728295 |
| hsa-miR-4294_st | 7.481153293 | 6.91149177 | 7.558529339 | 7.244048976 | 7.621170716 | 7.799023059 | 7.655182395 |
| hsa-miR-4301_st | 2.818798622 | 2.60645247 | 2.78892964 | 2.345675164 | 0.522742731 | 1.074041925 | 2.128799987 |
| hsa-miR-4299_st | 12.05659727 | 12.09834933 | 12.66421984 | 12.65788685 | 13.0334779 | 13.51867114 | 13.3246008 |
| hsa-miR-4298_st | 12.58812349 | 12.53573197 | 12.35668678 | 12.78107448 | 12.6480775 | 13.4060123 | 13.12797181 |
| hsa-miR-4300_st | 7.161391413 | 6.95567403 | 6.037551085 | 6.57196457 | 7.810743774 | 8.064461286 | 7.755579197 |
| hsa-miR-4304_st | 6.089357109 | 5.701927756 | 6.059980055 | 5.644317816 | 6.088406496 | 6.340026321 | 5.420582731 |
| hsa-miR-4302_st | 5.963622133 | 5.515523129 | 4.766567559 | 4.495000852 | 5.706249065 | 5.494622234 | 5.111962397 |
| hsa-miR-4303_st | 6.111169503 | 5.440604988 | 6.001063779 | 6.000298409 | 6.590324392 | 6.364861429 | 6.62831735 |
| hsa-miR-4305_st | 4.202856017 | 3.837546276 | 2.961465968 | 3.677286193 | 4.46110794 | 2.332976389 | 3.304915114 |
| hsa-miR-4306_st | 9.282911039 | 8.940876277 | 10.03554276 | 9.726933704 | 9.791884762 | 9.166164656 | 9.280134261 |
| hsa-miR-4309_st | 3.351716667 | 3.915182995 | 3.119480508 | 4.081796029 | 3.06432113 | 3.384715826 | 3.568271745 |
| hsa-miR-4307_st | 3.446013852 | 2.705881622 | 3.218497829 | 2.848625951 | 2.505498813 | 2.347619972 | 2.40996454 |
| hsa-miR-4308_st | 1.81160017 | 0.900775218 | 3.95836533 | 2.569646273 | 4.736076769 | 1.698102701 | −0.050242221 |
| hsa-miR-4310_st | 3.61591216 | 3.406759551 | 3.534973865 | 3.200110899 | 3.986172412 | 3.428936167 | 4.68705003 |
| hsa-miR-4311_st | 5.268279971 | 4.506457653 | 4.783057011 | 4.762950871 | 5.083185755 | 4.195793025 | 4.722818483 |
| hsa-miR-4312_st | 6.158806152 | 5.562110779 | 5.674927687 | 6.046393989 | 7.083683585 | 6.618348836 | 7.155204626 |
| hsa-miR-4313_st | 5.774298155 | 5.744425037 | 5.070700374 | 4.860930492 | 5.545820369 | 5.632840694 | 5.02406122 |
| hsa-miR-4315_st | 3.832077164 | 3.376791457 | 3.613141601 | 1.315425646 | 3.261485823 | 3.542305385 | 2.119148903 |
| hsa-miR-4316_st | 7.011870998 | 7.137774779 | 6.595361143 | 6.953089155 | 7.282161377 | 7.580968943 | 7.186343162 |
| hsa-miR-4314_st | 8.424165966 | 7.951440979 | 7.940952393 | 8.547408149 | 8.873631299 | 8.672230049 | 8.463766145 |
| hsa-miR-4318_st | 3.260461495 | 2.920004471 | 3.362836281 | 3.771869026 | 2.434533697 | 2.509148747 | 2.930914093 |
| hsa-miR-4319_st | 3.773306123 | 2.90423951 | 3.299110309 | 1.870652131 | 4.625432568 | 3.222991464 | 3.862592832 |
| hsa-miR-4320_st | 2.564673468 | 2.997114923 | 2.947977616 | 2.33115349 | 1.344200071 | 2.176769863 | 2.770817374 |
| hsa-miR-4317_st | 8.923029572 | 8.87079085 | 8.813434135 | 9.16409292 | 8.975067067 | 9.202460064 | 9.686813667 |
| hsa-miR-4322_st | 12.15044048 | 11.75827297 | 11.21239516 | 11.73918168 | 11.84706877 | 12.9039712 | 12.4326584 |
| hsa-miR-4321_st | 10.06733764 | 8.570222806 | 8.774261434 | 9.654911362 | 9.33442321 | 10.43809905 | 10.081932 |
| hsa-miR-4323_st | 5.611698061 | 5.129023136 | 5.323666992 | 4.859042311 | 6.276210215 | 6.025180546 | 5.272196406 |
| hsa-miR-4324_st | 7.197692236 | 7.264416684 | 7.763840103 | 8.697732169 | 6.289309834 | 6.414060472 | 7.480767725 |
| hsa-miR-4256_st | 3.778191253 | 3.484732192 | 3.433191495 | 3.729854543 | 3.666361754 | 3.980854038 | 3.570092295 |
| hsa-miR-4257_st | 11.61630969 | 11.22624102 | 11.173811 | 11.66483233 | 11.95937863 | 12.43951835 | 12.42531151 |
| hsa-miR-4258_st | 7.05684659 | 6.758624713 | 6.706779888 | 7.110311519 | 7.366696705 | 7.974619474 | 7.564725439 |
| hsa-miR-4259_st | 10.31612181 | 10.0972969 | 10.04688495 | 10.41011934 | 10.52843707 | 11.28390392 | 11.20459012 |
| hsa-miR-4260_st | 9.934255683 | 9.6974814 | 9.497927139 | 10.05243426 | 10.38740469 | 11.26290711 | 10.84894645 |
| hsa-miR-4253_st | 10.94325295 | 10.53486344 | 9.382332208 | 10.77654639 | 10.30130979 | 11.87002694 | 11.0720852 |
| hsa-miR-4251_st | 5.959184496 | 5.145460752 | 5.641174335 | 5.082869293 | 5.690303682 | 6.495687146 | 6.103203421 |
| hsa-miR-4254_st | 5.263434455 | 5.406242379 | 5.353957445 | 4.276094418 | 5.336639541 | 5.398111164 | 5.747805442 |
| hsa-miR-4255_st | 2.779201757 | 2.682653308 | 2.353279478 | 2.440565985 | 2.67720195 | 2.709991132 | 2.931546048 |
| hsa-miR-4252_st | 4.954896134 | 4.36412946 | 4.608268232 | 4.710064558 | 4.726923841 | 4.929985145 | 4.89251963 |
| hsa-miR-4325_st | 2.581088587 | 3.15643584 | 3.657105249 | 3.163869184 | 2.898559949 | 2.712018596 | 2.99310898 |
| hsa-miR-4326_st | 4.519922416 | 4.215471803 | 5.15894637 | 5.198679542 | 5.259475575 | 5.17004873 | 4.568383986 |
| hsa-miR-4327_st | 11.4785337 | 11.13289466 | 10.88493226 | 11.54716271 | 11.31150484 | 12.35103234 | 12.07234527 |
| hsa-miR-4261_st | 8.27941676 | 8.252080117 | 8.454613797 | 8.342882591 | 8.541538972 | 8.532189392 | 8.424020402 |
| hsa-miR-4265_st | 7.230204994 | 6.751223585 | 6.995385817 | 6.926814437 | 8.008516636 | 8.130377128 | 7.075633329 |
| hsa-miR-4266_st | 5.737945221 | 5.205356228 | 4.950480238 | 5.150353387 | 5.801653653 | 6.391334331 | 5.876488304 |
| hsa-miR-4267_st | 5.414966575 | 5.237375149 | 5.356802768 | 5.282128524 | 5.058805771 | 6.215340561 | 5.979414082 |
| hsa-miR-4262_st | 1.11352019 | 2.010583297 | 1.404089399 | 3.361448698 | 0.933191434 | 0.499419015 | 1.411694108 |
| hsa-miR-2355_st | 2.451564924 | 3.005322707 | 1.752137736 | 2.375507986 | 1.881761575 | 2.316176853 | 2.349611677 |
| hsa-miR-4268_st | 3.878892166 | 3.383421912 | 3.30870814 | 4.409794591 | 3.836592396 | 3.66462124 | 3.651450901 |
| hsa-miR-4269_st | 7.647573844 | 7.361324193 | 7.350295586 | 7.474638443 | 7.591727581 | 8.243208918 | 7.295971325 |
| hsa-miR-4263_st | 2.897367252 | 2.854330203 | 3.198309191 | 2.916433852 | 2.54952666 | 2.1106731 | 1.946336618 |
| hsa-miR-4264_st | 2.360459661 | 2.555651444 | 1.662711397 | 2.635492247 | 2.901200782 | 2.398923091 | 2.194039083 |
| hsa-miR-4270_st | 14.76903588 | 14.8977843 | 14.28603806 | 14.86834796 | 14.33858693 | 15.71612339 | 15.25085457 |
| hsa-miR-4271_st | 11.26552869 | 10.69345375 | 10.91885546 | 11.40453569 | 11.09625185 | 12.0996921 | 11.71406075 |
| hsa-miR-4272_st | 2.029967476 | 2.063486562 | 1.119464816 | 0.479548059 | −0.125264244 | 0.779872036 | 1.197038805 |
| hsa-miR-4273_st | 0.919750283 | 1.242349958 | 1.768366915 | 0.674529963 | 2.44533478 | 0.42295701 | 0.539418548 |
| hsa-miR-4276_st | 1.451202622 | 1.26008887 | 1.992381944 | 1.240573395 | 0.5047776 | 1.874571633 | 0.270794869 |
| hsa-miR-4275_st | 2.637497712 | 1.930413539 | 1.845608378 | 2.199785703 | 2.761088376 | 0.344462935 | 1.921899108 |
| hsa-miR-4274_st | 5.401284804 | 5.492092522 | 4.949995338 | 4.949286304 | 5.627987532 | 5.771016274 | 5.558610949 |
| hsa-miR-4281_st | 14.95015845 | 15.38928323 | 14.8154776 | 15.32409242 | 14.53295182 | 15.83054893 | 15.5561999 |
| hsa-miR-4277_st | 1.945066901 | 1.933134038 | 2.818428903 | 2.458360311 | 2.091420837 | 2.703888291 | 2.077394057 |
| hsa-miR-4279_st | 5.735195753 | 4.555957853 | 4.617077131 | 3.957327091 | 4.977750745 | 5.7672041 | 4.439094896 |
| hsa-miR-4278_st | 6.769430522 | 6.21230605 | 5.393414183 | 6.405381498 | 7.266283499 | 6.938920314 | 5.867434879 |
| hsa-miR-4280_st | 4.217511889 | 4.177986642 | 4.38336885 | 3.958048382 | 4.481304882 | 4.086782868 | 4.872075886 |
| hsa-miR-4282_st | 0.414003371 | 0.803873241 | −0.235498086 | −0.56519605 | −0.417528894 | 0.408227053 | −0.270863464 |
| hsa-miR-4285_st | 11.19117121 | 10.7788461 | 10.50026416 | 11.41126959 | 11.43660294 | 11.95910636 | 11.85641496 |
| hsa-miR-4283_st | 8.274272288 | 7.767795071 | 7.173974694 | 8.170720359 | 8.300815471 | 8.708288831 | 8.351162185 |
| hsa-miR-4284_st | 14.53463673 | 15.41007006 | 14.96709933 | 14.66963423 | 14.53079604 | 15.22239844 | 14.81581223 |
| hsa-miR-4286_st | 14.13825949 | 14.54600224 | 13.95215957 | 14.53970174 | 13.31428965 | 13.84741972 | 13.96222355 |
| hsa-miR-4287_st | 4.250551137 | 5.27016654 | 4.217148199 | 5.183834788 | 5.065523905 | 5.547011721 | 5.16394333 |
| hsa-miR-4288_st | 4.44515386 | 5.131605678 | 5.606988799 | 4.89996953 | 4.192276052 | 4.315456751 | 4.61593792 |
| hsa-miR-4292_st | 7.154388047 | 6.52788671 | 6.001495602 | 6.147113216 | 7.116515418 | 7.928765574 | 7.427084959 |
| hsa-miR-4289_st | 4.840611331 | 6.061450616 | 6.085938043 | 7.315498394 | 6.129057092 | 5.862832369 | 6.77065839 |
| hsa-miR-4290_st | 7.283698192 | 5.947016994 | 5.549512083 | 6.054037103 | 7.585893889 | 6.470195529 | 5.356387769 |
| hsa-miR-4291_st | 5.954604637 | 6.611914779 | 5.712379703 | 6.115296988 | 7.691933339 | 6.500027376 | 6.854483862 |
| hsa-miR-4329_st | 5.56478742 | 5.87164337 | 5.99687936 | 6.329544129 | 5.070309748 | 5.189209208 | 5.468667534 |
| hsa-miR-4330_st | 5.528112722 | 5.430515261 | 3.856110173 | 5.306409642 | 4.970604903 | 5.447401074 | 5.959457727 |
| hsa-miR-500b_st | 3.667298411 | 4.025630625 | 4.383865183 | 3.782416157 | 3.719773735 | 4.834009862 | 3.881715266 |
| hsa-miR-4328_st | 3.523442212 | 3.542135448 | 3.94829819 | 3.280353743 | 3.529277114 | 2.09856958 | 1.452698155 |

Hashimotyo's thyroiditis (HASH);
papillary carcinoma (PTC);
follicular variant of PTC (FVPTC);
follicular carcinoma (FTC);
oncocytic follicular carcinoma (OFTC);
anaplastic carcinoma (ATC medullary carcinoma (MED). [symbol]
indicates data missing or illegible when filed

TABLE 4C
normalized array data for the 55 benign and malignant FFPE samples

Average

| miRNA | Benign lesions | Malignant lesions |
|---|---|---|
| hsa-let-7a_st | 14.36000595 | 14.31198199 |
| hsa-let-7a-star_st | 1.817188905 | 2.034634364 |
| hsa-let-7a-2-star_st | 4.848130219 | 4.810523869 |
| hsa-let-7b_st | 13.45339038 | 13.46708924 |
| hsa-let-7b-star_st | 7.052169256 | 6.303827505 |
| hsa-let-7c_st | 14.0768108 | 14.00524749 |
| hsa-let-7c-star_st | 3.944675826 | 4.072642857 |
| hsa-let-7d_st | 13.81633445 | 13.89989481 |
| hsa-let-7d-star_st | 8.179506062 | 7.567053856 |
| hsa-let-7e_st | 12.2186856 | 12.5052487 |
| hsa-let-7e-star_st | 4.367109325 | 4.152463921 |
| hsa-let-7f_st | 13.84568839 | 13.8708236 |
| hsa-let-7f-1-star_st | 4.233470866 | 3.935010527 |
| hsa-let-7f-2-star_st | 1.757216137 | 2.23118618 |
| hsa-miR-15a_st | 9.523730359 | 10.63249938 |
| hsa-miR-15a-star_st | 3.917852022 | 4.085613882 |
| hsa-miR-16_st | 12.97462979 | 13.70944322 |
| hsa-miR-16-1-star_st | 2.99904911 | 3.740348404 |
| hsa-miR-17_st | 9.500384065 | 10.30568924 |
| hsa-miR-17-star_st | 5.283542505 | 5.698159331 |
| hsa-miR-18a_st | 4.141081926 | 5.032812284 |
| hsa-miR-18a-star_st | 4.795553175 | 4.884815074 |
| hsa-miR-19a-star_st | 0.685050416 | 0.532108606 |
| hsa-miR-19a_st | 4.143552632 | 4.838381769 |
| hsa-miR-19b-1-star_st | 2.451457654 | 2.621841261 |
| hsa-miR-19b_st | 9.006935767 | 9.689618053 |
| hsa-miR-19b-2-star_st | 1.018555034 | 1.137498593 |
| hsa-miR-20a_st | 8.89362477 | 9.588615579 |
| hsa-miR-20a-star_st | 5.959174041 | 5.710046197 |
| hsa-miR-21_st | 10.50865483 | 12.68198373 |
| hsa-miR-21-star_st | 5.807031546 | 6.720356935 |
| hsa-miR-22-star_st | 6.689072196 | 7.494085605 |
| hsa-miR-22_st | 11.52342875 | 12.0204796 |
| hsa-miR-23a-star_st | 10.63608279 | 10.23287553 |
| hsa-miR-23a_st | 13.81616627 | 14.41432249 |
| hsa-miR-24-1-star_st | 2.801615889 | 3.953883 |
| hsa-miR-24_st | 12.73350506 | 13.61325723 |
| hsa-miR-24-2-star_st | 4.615385083 | 6.158120747 |
| hsa-miR-25-star_st | 12.33778526 | 11.68803427 |
| hsa-miR-25_st | 10.19509648 | 10.49485004 |
| hsa-miR-26a_st | 14.61398465 | 14.48916527 |
| hsa-miR-26a-1-star_st | 1.854990302 | 1.453798647 |
| hsa-miR-26b_st | 11.29214694 | 11.52330198 |
| hsa-miR-26b-star_st | 4.850227976 | 5.065710184 |
| hsa-miR-27a-star_st | 4.965521181 | 5.286398414 |
| hsa-miR-27a_st | 11.22486946 | 12.53462951 |
| hsa-miR-28-5p_st | 7.868477936 | 8.605030726 |
| hsa-miR-28-3p_st | 6.205295102 | 6.674609956 |
| hsa-miR-29a-star_st | 1.574704733 | 1.841258599 |
| hsa-miR-29a_st | 12.84143789 | 13.58290488 |
| hsa-miR-30a_st | 12.02183057 | 12.10230006 |
| hsa-miR-30a-star_st | 9.961700524 | 8.854338003 |
| hsa-miR-31_st | 8.523069812 | 10.1455091 |
| hsa-miR-31-star_st | 2.222678966 | 4.612984691 |
| hsa-miR-32_st | 0.72558191 | 1.35229567 |
| hsa-miR-32-star_st | 2.970239513 | 2.776181659 |
| hsa-miR-33a_st | 0.37861614 | 0.6924047 |
| hsa-miR-33a-star_st | 2.101622678 | 3.023222763 |
| hsa-miR-92a-1-star_st | 8.017692542 | 7.436092652 |
| hsa-miR-92a_st | 10.65750464 | 10.80178948 |
| hsa-miR-92a-2-star_st | 9.102149194 | 8.505089146 |
| hsa-miR-93_st | 9.159293092 | 9.963608318 |
| hsa-miR-93-star_st | 4.376632867 | 4.973263034 |
| hsa-miR-95_st | 7.323539117 | 6.976585435 |
| hsa-miR-96_st | 4.900564549 | 5.940308881 |
| hsa-miR-96-star_st | 3.234687115 | 3.077770058 |
| hsa-miR-98_st | 8.966376043 | 9.888968078 |
| hsa-miR-99a_st | 11.92738151 | 12.44015979 |
| hsa-miR-99a-star_st | 4.070995307 | 4.952788995 |
| hsa-miR-100_st | 11.9698056 | 12.21937804 |
| hsa-miR-100-star_st | 2.17563109 | 1.904357889 |
| hsa-miR-101-star_st | 2.093249813 | 2.458609388 |
| hsa-miR-101_st | 4.781687089 | 5.603263242 |
| hsa-miR-29b-1-star_st | 5.619415955 | 6.085087062 |
| hsa-miR-29b_st | 10.71743423 | 11.66840776 |
| hsa-miR-29b-2-star_st | 7.591698518 | 7.664428275 |
| hsa-miR-103-2-star_st | 4.636666583 | 5.116206973 |
| hsa-miR-103_st | 12.05908493 | 12.55126282 |
| hsa-miR-105_st | 2.00205694 | 2.014277348 |
| hsa-miR-105-star_st | 2.876309165 | 2.704470738 |
| hsa-miR-106a_st | 9.678844073 | 10.32064486 |
| hsa-miR-106a-star_st | 1.313898069 | 1.311817168 |
| hsa-miR-107_st | 11.83968896 | 12.41731811 |
| hsa-miR-16-2-star_st | 3.100002226 | 3.990160093 |
| hsa-miR-192_st | 6.616238334 | 7.209948907 |
| hsa-miR-192-star_st | 3.51811829 | 3.737829775 |
| hsa-miR-196a_st | 2.205417441 | 3.202438077 |
| hsa-miR-197_st | 7.752098414 | 7.05667193 |
| hsa-miR-198_st | 12.20408767 | 11.82988454 |
| hsa-miR-199a-5p_st | 9.09156334 | 10.01581872 |
| hsa-miR-199a-3p_st | 10.41531984 | 11.23108944 |
| hsa-miR-208a_st | 2.426923226 | 2.866876966 |
| hsa-miR-129-5p_st | 11.37107956 | 10.73008473 |
| hsa-miR-129-star_st | 3.990282192 | 4.219661375 |
| hsa-miR-148a-star_st | 4.318157258 | 4.562739552 |
| hsa-miR-148a_st | 9.727259974 | 10.55115809 |
| hsa-miR-30c_st | 13.21568699 | 12.99368195 |
| hsa-miR-30c-2-star_st | 7.508318158 | 7.105102486 |
| hsa-miR-30d_st | 11.80359334 | 12.17102503 |
| hsa-miR-30d-star_st | 2.07699433 | 2.351079747 |
| hsa-miR-139-5p_st | 7.018950578 | 5.92709119 |
| hsa-miR-139-3p_st | 7.694936312 | 7.147328758 |
| hsa-miR-147_st | 4.165365984 | 3.90638249 |
| hsa-miR-7_st | 8.427198768 | 6.266205738 |
| hsa-miR-7-1-star_st | 5.912670765 | 6.303490609 |
| hsa-miR-7-2-star_st | 6.588741297 | 5.697015591 |
| hsa-miR-10a_st | 7.611506522 | 8.371969516 |
| hsa-miR-10a-star_st | 3.996873331 | 4.184409763 |
| hsa-miR-10b_st | 8.299153562 | 9.492010377 |
| hsa-miR-10b-star_st | 3.623172296 | 3.897742826 |
| hsa-miR-34a_st | 8.639499617 | 10.28404006 |
| hsa-miR-34a-star_st | 4.948757717 | 6.458906078 |
| hsa-miR-181a_st | 11.8162237 | 12.30958671 |
| hsa-miR-181a-2-star_st | 7.49108462 | 8.51734465 |
| hsa-miR-181b_st | 10.54596321 | 11.07333877 |
| hsa-miR-181c_st | 7.714549502 | 8.726418322 |
| hsa-miR-181c-star_st | 6.15088745 | 6.749584529 |
| hsa-miR-182_st | 7.854141892 | 9.116388045 |
| hsa-miR-182-star_st | 2.071525858 | 1.728213696 |
| hsa-miR-183_st | 5.037678916 | 5.668334307 |
| hsa-miR-183-star_st | 6.8335086 | 6.70005607 |
| hsa-miR-187-star_st | 9.653086446 | 9.162341913 |
| hsa-miR-187_st | 3.832697742 | 4.116541122 |
| hsa-miR-196a-star_st | 4.139255612 | 4.713887099 |
| hsa-miR-199b-5p_st | 5.734094768 | 6.66582012 |
| hsa-miR-199b-3p_st | 10.2965265 | 11.30516471 |
| hsa-miR-203_st | 6.463218637 | 6.498685112 |
| hsa-miR-204_st | 7.152259387 | 5.50936759 |
| hsa-miR-205_st | 4.167524329 | 4.532847116 |
| hsa-miR-205-star_st | 4.928554113 | 4.828048623 |
| hsa-miR-210_st | 12.49990996 | 12.07385469 |
| hsa-miR-211_st | 5.161069512 | 4.953134599 |
| hsa-miR-212_st | 5.850386233 | 6.237663924 |
| hsa-miR-181a-star_st | 4.446572243 | 5.664004911 |
| hsa-miR-214-star_st | 4.661216959 | 5.325425233 |
| hsa-miR-214_st | 10.26091254 | 10.06821253 |
| hsa-miR-215_st | 3.692075806 | 4.452383278 |
| hsa-miR-216a_st | 2.006819738 | 2.215754821 |
| hsa-miR-217_st | 2.337664425 | 2.63315841 |
| hsa-miR-218_st | 3.63808555 | 4.853644376 |
| hsa-miR-218-1-star_st | 1.961569664 | 1.558810001 |
| hsa-miR-218-2-star_st | 3.307895039 | 2.95864268 |
| hsa-miR-219-5p_st | 0.558983952 | 0.582593709 |
| hsa-miR-219-1-3p_st | 4.353658641 | 4.606037137 |
| hsa-miR-220a_st | 2.04233455 | 2.155153308 |
| hsa-miR-221-star_st | 5.351253871 | 8.067211581 |
| hsa-miR-221_st | 11.92279588 | 13.8201505 |
| hsa-miR-222-star_st | 5.725998902 | 6.531098523 |
| hsa-miR-222_st | 11.65865636 | 13.62259088 |
| hsa-miR-223-star_st | 2.709788027 | 3.24220149 |
| hsa-miR-223_st | 8.411456875 | 9.035877876 |
| hsa-miR-224_st | 6.111303772 | 7.102224934 |
| hsa-miR-224-star_st | 5.065366846 | 5.377721206 |
| hsa-miR-200b-star_st | 6.396031008 | 5.991071351 |
| hsa-miR-200b_st | 10.44380414 | 10.62028303 |
| hsa-let-7g_st | 13.06680131 | 13.24754752 |
| hsa-let-7g-star_st | 5.228588386 | 4.868016299 |
| hsa-let-7i_st | 13.40307965 | 13.81911381 |
| hsa-let-7i-star_st | 5.725209102 | 6.113275002 |
| hsa-miR-1_st | 3.605902148 | 3.667255452 |
| hsa-miR-15b_st | 10.25425488 | 10.25040802 |
| hsa-miR-15b-star_st | 3.073962052 | 2.547676394 |
| hsa-miR-23b-star_st | 5.181113699 | 5.135602016 |
| hsa-miR-23b_st | 13.98359691 | 14.43745778 |
| hsa-miR-27b-star_st | 5.701586643 | 6.68992006 |
| hsa-miR-27b_st | 11.51233731 | 12.4572577 |
| hsa-miR-30b_st | 11.74389887 | 12.14485003 |
| hsa-miR-30b-star_st | 8.81777762 | 8.371591203 |
| hsa-miR-122_st | 3.646502561 | 3.11823204 |
| hsa-miR-122-star_st | 1.619469872 | 1.837565558 |
| hsa-miR-124-star_st | 4.93211131 | 4.833770967 |
| hsa-miR-124_st | 7.972483773 | 7.801098825 |
| hsa-miR-125b_st | 14.37624126 | 14.44963404 |
| hsa-miR-125b-1-star_st | 11.75832583 | 11.3109495 |
| hsa-miR-128_st | 7.527388925 | 7.902519075 |
| hsa-miR-130a-star_st | 1.538371677 | 1.462469102 |
| hsa-miR-130a_st | 10.9465824 | 11.06460559 |
| hsa-miR-132-star_st | 2.372952819 | 3.369454077 |
| hsa-miR-132_st | 6.097848644 | 7.163642343 |
| hsa-miR-133a_st | 5.067413225 | 4.642066769 |
| hsa-miR-135a_st | 1.15975216 | 2.22073332 |
| hsa-miR-135a-star_st | 12.22633278 | 11.72121432 |
| hsa-miR-137_st | 1.255264255 | 1.492345231 |
| hsa-miR-138_st | 10.89537098 | 9.93510521 |
| hsa-miR-138-2-star_st | 3.820287956 | 4.076322371 |
| hsa-miR-140-5p_st | 4.091314417 | 5.22161458 |
| hsa-miR-140-3p_st | 10.50572604 | 10.72876276 |
| hsa-miR-141-star_st | 2.211640062 | 2.203296777 |
| hsa-miR-141_st | 8.444729901 | 8.89520913 |
| hsa-miR-142-5p_st | 3.357937041 | 4.711509093 |
| hsa-miR-142-3p_st | 0.584297115 | 0.830877789 |
| hsa-miR-143-star_st | 6.431750149 | 6.366869822 |
| hsa-miR-143_st | 11.96506029 | 12.21085325 |
| hsa-miR-144-star_st | 2.589339021 | 2.615721951 |
| hsa-miR-144_st | 1.257976317 | 0.783692809 |
| hsa-miR-145_st | 13.19010848 | 12.85861061 |
| hsa-miR-145-star_st | 4.996042403 | 5.285051949 |
| hsa-miR-152_st | 9.860515062 | 9.72840712 |
| hsa-miR-153_st | 1.39098679 | 1.597191112 |
| hsa-miR-191_st | 12.39382767 | 12.92127283 |
| hsa-miR-191-star_st | 6.045325279 | 5.868049182 |
| hsa-miR-9_st | −0.037423131 | 0.323639859 |
| hsa-miR-9-star_st | 1.862780771 | 1.992323554 |
| hsa-miR-125a-5p_st | 13.24316542 | 13.27096846 |
| hsa-miR-125a-3p_st | 11.86938739 | 11.51258355 |
| hsa-miR-125b-2-star_st | 9.209754481 | 9.008151793 |
| hsa-miR-126-star_st | 0.587084425 | 1.16320053 |
| hsa-miR-126_st | 13.11731956 | 13.11019041 |
| hsa-miR-127-5p_st | 5.708661536 | 6.148581495 |
| hsa-miR-127-3p_st | 5.963094836 | 6.502955865 |
| hsa-miR-129-3p_st | 2.925722079 | 3.577022621 |
| hsa-miR-134_st | 8.238898408 | 7.975684141 |
| hsa-miR-136_st | 0.436691213 | 0.750769397 |
| hsa-miR-136-star_st | 1.18821308 | 1.646729981 |
| hsa-miR-138-1-star_st | 7.63398959 | 6.723501115 |
| hsa-miR-146a_st | 8.000275818 | 9.489633492 |
| hsa-miR-146a-star_st | 0.599256679 | 1.438452988 |
| hsa-miR-149_st | 6.561947984 | 6.824176008 |
| hsa-miR-149-star_st | 15.94959873 | 15.45479213 |
| hsa-miR-150_st | 9.786207 | 9.650047292 |
| hsa-miR-150-star_st | 13.05492785 | 12.70584759 |
| hsa-miR-154_st | 3.877395393 | 3.839602042 |
| hsa-miR-154-star_st | 3.206254747 | 2.993683876 |
| hsa-miR-184_st | 7.42739076 | 7.018936933 |
| hsa-miR-185_st | 9.38739608 | 9.269716021 |
| hsa-miR-185-star_st | 9.740932048 | 8.766964677 |
| hsa-miR-186_st | 4.442838044 | 4.275145719 |
| hsa-miR-186-star_st | 0.087196561 | 0.839547882 |
| hsa-miR-188-5p_st | 6.728698341 | 6.882128003 |
| hsa-miR-188-3p_st | 3.474517048 | 3.608866094 |
| hsa-miR-190_st | −0.053777817 | −0.299015248 |
| hsa-miR-193a-5p_st | 12.47197392 | 11.63882799 |
| hsa-miR-193a-3p_st | 6.690228177 | 6.929106052 |
| hsa-miR-194_st | −6.702817284 | 7.08213949 |
| hsa-miR-195_st | 10.65287639 | 10.99785423 |
| hsa-miR-195-star_st | 4.719264269 | 5.119920239 |
| hsa-miR-206_st | 5.356578578 | 5.440984838 |
| hsa-miR-320a_st | 12.32287691 | 12.01672633 |
| hsa-miR-200c-star_st | 3.403121493 | 3.35018893 |
| hsa-miR-200c_st | 13.30675633 | 13.06693526 |
| hsa-miR-155_st | 8.442115751 | 9.050269641 |
| hsa-miR-155-star_st | 2.019873803 | 2.076690306 |
| hsa-miR-194-star_st | 8.342531305 | 7.827253713 |
| hsa-miR-106b_st | 8.645144215 | 9.541269309 |
| hsa-miR-106b-star_st | 7.409394541 | 7.546819664 |
| hsa-miR-29c-star_st | 7.149799152 | 7.290572146 |
| hsa-miR-29c_st | 10.79483359 | 11.23178028 |
| hsa-miR-30c-1-star_st | 10.447203 | 10.39730286 |
| hsa-miR-200a-star_st | 5.731292411 | 5.988460145 |
| hsa-miR-200a_st | 9.169758229 | 9.503851262 |
| hsa-miR-302a-star_st | 2.372057591 | 2.370495167 |
| hsa-miR-302a_st | 1.855387217 | 2.038060651 |
| hsa-miR-219-2-3p_st | 6.739852398 | 6.74831764 |
| hsa-miR-34b-star_st | 3.840904487 | 5.21965384 |
| hsa-miR-34b_st | 6.547213084 | 5.942852905 |
| hsa-miR-34c-5p_st | 2.090522064 | 2.678820887 |
| hsa-miR-34c-3p_st | 6.086653049 | 5.905086837 |
| hsa-miR-299-5p_st | 5.363201505 | 5.203675845 |
| hsa-miR-299-3p_st | 6.959936198 | 6.82563734 |
| hsa-miR-301a_st | 3.051935466 | 4.532745543 |
| hsa-miR-99b_st | 10.70845538 | 11.15355835 |
| hsa-miR-99b-star_st | 7.977755574 | 7.721754645 |
| hsa-miR-296-5p_st | 3.825898995 | 4.157035884 |
| hsa-miR-296-3p_st | 11.84964963 | 11.36519002 |
| hsa-miR-130b-star_st | 4.426314454 | 3.997836205 |
| hsa-miR-130b_st | 7.786698895 | 8.06324078 |
| hsa-miR-30e_st | 9.146166551 | 9.513823359 |
| hsa-miR-30e-star_st | 8.94504654 | 8.420921138 |
| hsa-miR-26a-2-star_st | 0.448195516 | 0.886994323 |
| hsa-miR-361-5p_st | 10.63653492 | 10.82243064 |
| hsa-miR-361-3p_st | 5.338284351 | 5.619177786 |
| hsa-miR-362-5p_st | 6.457708861 | 6.747031759 |
| hsa-miR-362-3p_st | 2.549692831 | 3.482225846 |
| hsa-miR-363-star_st | 10.16128719 | 9.740192497 |
| hsa-miR-363_st | 4.586469182 | 5.158970832 |
| hsa-miR-365_st | 2.846214258 | 2.76223775 |
| hsa-miR-365-star_st | 11.24944871 | 10.70587958 |
| hsa-miR-302b-star_st | 3.539467751 | 3.460255223 |
| hsa-miR-302b_st | −0.263529977 | 0.53926022 |
| hsa-miR-302c-star_st | 7.863854974 | 7.319376018 |
| hsa-miR-302c_st | 1.216885909 | 1.718478687 |
| hsa-miR-302d-star_st | 6.498846388 | 6.395437343 |
| hsa-miR-302d_st | 0.714797032 | 1.194972082 |
| hsa-miR-367-star_st | 0.877941639 | 0.877505077 |
| hsa-miR-367_st | 1.421065963 | 1.397357436 |
| hsa-miR-376c_st | 3.565402716 | 4.789057213 |
| hsa-miR-369-5p_st | 2.905969783 | 3.759062494 |
| hsa-miR-369-3p_st | 0.918124726 | 0.537099381 |
| hsa-miR-370_st | 11.03403163 | 10.54839769 |
| hsa-miR-371-5p_st | 11.89257045 | 11.19298009 |
| hsa-miR-371-3p_st | 4.502810974 | 3.735324728 |
| hsa-miR-372_st | 2.868332675 | 2.849718395 |
| hsa-miR-373-star_st | 11.17516631 | 10.26133923 |
| hsa-miR-373_st | 4.640220277 | 4.701820472 |
| hsa-miR-374a_st | 2.467623526 | 2.96858071 |
| hsa-miR-374a-star_st | 2.604831429 | 2.597379957 |
| hsa-miR-375_st | 4.398265966 | 6.827059343 |
| hsa-miR-376a-star_st | 0.507486272 | 0.392805453 |
| hsa-miR-376a_st | 3.313087786 | 3.970213789 |
| hsa-miR-377-star_st | 4.020741665 | 4.154103103 |
| hsa-miR-377_st | 2.068010287 | 2.61569932 |
| hsa-miR-378-star_st | 5.247508878 | 5.110442055 |
| hsa-miR-378_st | 8.410030576 | 8.522842373 |
| hsa-miR-379_st | 5.171820275 | 5.46418133 |
| hsa-miR-379-star_st | 2.342551084 | 2.04349047 |
| hsa-miR-380-star_st | 1.956654661 | 1.696314678 |
| hsa-miR-380_st | 0.986550906 | 0.551846428 |
| hsa-miR-381_st | 5.01019789 | 5.256109977 |
| hsa-miR-382_st | 6.686858479 | 6.752161838 |
| hsa-miR-383_st | 5.73743282 | 5.619950727 |
| hsa-miR-340_st | 2.759206667 | 4.245817534 |
| hsa-miR-340-star_st | 1.231739032 | 1.606564863 |
| hsa-miR-330-5p_st | 5.671343998 | 5.840636639 |
| hsa-miR-330-3p_st | 6.043678256 | 6.295562293 |
| hsa-miR-328_st | 7.843122583 | 7.298096324 |
| hsa-miR-342-5p_st | 5.148144481 | 5.065987529 |
| hsa-miR-342-3p_st | 9.812784392 | 10.37863789 |
| hsa-miR-337-5p_st | 2.723879131 | 2.765987114 |
| hsa-miR-337-3p_st | 3.853089832 | 4.011466473 |
| hsa-miR-323-5p_st | 11.02897044 | 10.51777655 |
| hsa-miR-323-3p_st | 3.303864373 | 3.924228136 |
| hsa-miR-326_st | 6.919923767 | 6.514124006 |
| hsa-miR-151-5p_st | 11.01794237 | 11.41683181 |
| hsa-miR-151-3p_st | 7.931115259 | 8.705733794 |
| hsa-miR-135b_st | 1.341991211 | 2.074222341 |
| hsa-miR-135b-star_st | 6.231949017 | 6.246922171 |
| hsa-miR-148b-star_st | 1.785330831 | 2.205730565 |
| hsa-miR-148b_st | 8.253236805 | 8.537720847 |
| hsa-miR-331-5p_st | 5.996544431 | 6.293113779 |
| hsa-miR-331-3p_st | 8.911242463 | 9.333572676 |
| hsa-miR-324-5p_st | 8.218170272 | 8.790495408 |
| hsa-miR-324-3p_st | 8.246863486 | 8.246251943 |
| hsa-miR-338-5p_st | 4.598152191 | 5.531917463 |
| hsa-miR-338-3p_st | 3.625466221 | 4.546999732 |
| hsa-miR-339-5p_st | 7.904657223 | 8.03347372 |
| hsa-miR-339-3p_st | 8.428769453 | 8.525128595 |
| hsa-miR-335_st | 7.349563245 | 8.63764613 |
| hsa-miR-335-star_st | 2.579882162 | 2.822031762 |
| hsa-miR-133b_st | 5.507453349 | 5.063207234 |
| hsa-miR-325_st | 3.374977587 | 3.506824457 |
| hsa-miR-345_st | 9.947792337 | 9.575654547 |
| hsa-miR-346_st | 9.252844035 | 8.515788731 |
| hsa-miR-384_st | 0.705946878 | 0.749499399 |
| hsa-miR-196b_st | 2.261522752 | 2.507888206 |
| hsa-miR-196b-star_st | 6.115599869 | 6.100450539 |
| hsa-miR-422a_st | 8.970061907 | 8.781848497 |
| hsa-miR-423-5p_st | 13.22560469 | 12.7766514 |
| hsa-miR-423-3p_st | 9.444694731 | 9.515284512 |
| hsa-miR-424_st | 5.173778206 | 6.558926603 |
| hsa-miR-424-star_st | 8.255107886 | 8.110617308 |
| hsa-miR-425_st | 7.675481843 | 9.000293305 |
| hsa-miR-425-star_st | 7.98460387 | 7.945211866 |
| hsa-miR-18b_st | 3.527288828 | 3.873630423 |
| hsa-miR-18b-star_st | 4.115489988 | 4.419179605 |
| hsa-miR-20b_st | 7.573277105 | 8.283015486 |
| hsa-miR-20b-star_st | 4.727461997 | 4.792609471 |
| hsa-miR-448_st | 2.103855381 | 2.210141362 |
| hsa-miR-429_st | 4.515379148 | 5.511999804 |
| hsa-miR-449a_st | 3.110645728 | 3.277116245 |
| hsa-miR-450a_st | 2.551079533 | 3.324852066 |
| hsa-miR-431_st | 2.550852409 | 3.004319505 |
| hsa-miR-431-star_st | 6.268771623 | 6.321666885 |
| hsa-miR-433_st | 4.554910789 | 4.969801894 |
| hsa-miR-329_st | 2.746201709 | 3.877841069 |
| hsa-miR-451_st | 12.5547106 | 12.02445188 |
| hsa-miR-452_st | 5.784024409 | 6.550204222 |
| hsa-miR-452-star_st | 3.622743958 | 3.186990324 |
| hsa-miR-409-5p_st | 3.330430107 | 3.623972951 |
| hsa-miR-409-3p_st | 5.503185818 | 5.942186892 |
| hsa-miR-412_st | 4.206326729 | 4.362758974 |
| hsa-miR-410_st | 3.259684019 | 3.643967998 |
| hsa-miR-376b_st | 1.828883403 | 2.459601136 |
| hsa-miR-483-5p_st | 11.40324762 | 11.06001912 |
| hsa-miR-483-3p_st | 6.916828196 | 6.522209625 |
| hsa-miR-484_st | 7.171207884 | 7.393697906 |
| hsa-miR-485-5p_st | 6.00157946 | 6.034655041 |
| hsa-miR-485-3p_st | 5.39893985 | 5.441357088 |
| hsa-miR-486-5p_st | 8.817967119 | 7.680934994 |
| hsa-miR-486-3p_st | 10.16038619 | 9.324192829 |
| hsa-miR-487a_st | 5.190894234 | 5.234422767 |
| hsa-miR-488-star_st | 2.037661041 | 2.031702259 |
| hsa-miR-488_st | 1.917044666 | 2.074173654 |
| hsa-miR-489_st | 8.065921716 | 8.172967587 |
| hsa-miR-490-5p_st | 6.754727362 | 6.554242182 |
| hsa-miR-490-3p_st | 4.591119855 | 4.858750807 |
| hsa-miR-491-5p_st | 9.174267442 | 8.827885194 |
| hsa-miR-491-3p_st | 4.817072369 | 4.756033597 |
| hsa-miR-511_st | 0.582438965 | 0.716293373 |
| hsa-miR-146b-5p_st | 8.65119709 | 12.64738009 |
| hsa-miR-146b-3p_st | 3.914402013 | 6.123201923 |
| hsa-miR-202-star_st | −0.781474877 | −0.007931765 |
| hsa-miR-202_st | 8.491867741 | 8.241607401 |
| hsa-miR-492_st | 7.956878031 | 7.899261836 |
| hsa-miR-493-star_st | 3.108410034 | 4.277607706 |
| hsa-miR-493_st | 4.977110017 | 4.958178529 |
| hsa-miR-432_st | 6.760195763 | 6.744838142 |
| hsa-miR-432-star_st | 4.779154597 | 4.709849583 |
| hsa-miR-494_st | 14.93967888 | 14.97979071 |
| hsa-miR-495_st | 2.336665394 | 3.336559005 |
| hsa-miR-496_st | 1.467169895 | 1.393316839 |
| hsa-miR-193b-star_st | 11.48400641 | 10.73433048 |
| hsa-miR-193b_st | 7.937207432 | 8.284766042 |
| hsa-miR-497_st | 8.895613769 | 9.029656096 |
| hsa-miR-497-star_st | 2.281978967 | 1.832596009 |
| hsa-miR-181d_st | 9.070447864 | 9.349844873 |
| hsa-miR-512-5p_st | 4.259904447 | 4.147121496 |
| hsa-miR-512-3p_st | 4.586526379 | 4.211378463 |
| hsa-miR-498_st | 11.35242614 | 10.80489493 |
| hsa-miR-520e_st | 1.204578854 | 1.686790871 |
| hsa-miR-515-5p_st | 1.970304748 | 2.393561669 |
| hsa-miR-515-3p_st | 3.126241548 | 3.481186768 |
| hsa-miR-519c-star_st | 5.720072666 | 5.589378134 |
| hsa-miR-519c_st | 3.71700652 | 3.797069026 |
| hsa-miR-520f_st | 1.131815056 | 1.691206685 |
| hsa-miR-519c-5p_st | 5.309746638 | 5.003525016 |
| hsa-miR-519c-3p_st | 0.22969516 | −0.113714668 |
| hsa-miR-520a-5p_st | 3.638243827 | 3.334189211 |
| hsa-miR-520a-3p_st | 2.228775692 | 2.327561233 |
| hsa-miR-526b_st | 5.017040378 | 4.595789821 |
| hsa-miR-526b-star_st | 0.96461501 | 1.2883939 |
| hsa-miR-519b-5p_st | 5.816569346 | 5.485221487 |
| hsa-miR-519b-3p_st | 1.582189453 | 1.35015253 |
| hsa-miR-525-5p_st | 5.25469618 | 4.941976647 |
| hsa-miR-525-3p_st | 2.904452936 | 2.63710265 |
| hsa-miR-523-star_st | 5.223994896 | 4.55164185 |
| hsa-miR-523_st | 1.349865804 | 1.309785593 |
| hsa-miR-518f-star_st | 5.975454364 | 5.920854764 |
| hsa-miR-518f_st | 1.138901913 | 1.124159574 |
| hsa-miR-520b_st | 1.179636607 | 0.989989281 |
| hsa-miR-518b_st | 3.744046243 | 3.185822688 |
| hsa-miR-526a_st | 5.909151007 | 5.690469241 |
| hsa-miR-520c-5p_st | 5.516401921 | 6.084709544 |
| hsa-miR-520c-3p_st | 3.15184483 | 3.214547385 |
| hsa-miR-518c-star_st | 8.252522272 | 7.797982062 |
| hsa-miR-518c_st | 0.233635096 | 0.146131446 |
| hsa-miR-524-5p_st | 4.635589471 | 4.681020416 |
| hsa-miR-524-3p_st | 3.176089486 | 2.800846584 |
| hsa-miR-517-star_st | 4.629680738 | 4.429839016 |
| hsa-miR-517a_st | 1.876262279 | 1.136074773 |
| hsa-miR-519d_st | 3.627373355 | 3.599347618 |
| hsa-miR-521_st | 2.872418246 | 2.90502578 |
| hsa-miR-520d-5p_st | 5.804831501 | 5.831473806 |
| hsa-miR-520d-3p_st | 4.879582049 | 5.286354066 |
| hsa-miR-517b_st | 3.076315007 | 2.846741496 |
| hsa-miR-520g_st | 2.361015476 | 2.048745141 |
| hsa-miR-516b_st | 6.666902208 | 6.144496808 |
| hsa-miR-516b-star_st | 2.782027252 | 3.003735243 |
| hsa-miR-518e-star_st | 6.014418927 | 5.786884935 |
| hsa-miR-518e_st | 2.009867794 | 1.595249971 |
| hsa-miR-518a-5p_st | 6.341247965 | 6.348362585 |
| hsa-miR-518a-3p_st | 3.034809147 | 2.919010517 |
| hsa-miR-518d-5p_st | 5.933174177 | 5.92772073 |
| hsa-miR-518d-3p_st | 2.643191913 | 2.696261216 |
| hsa-miR-517c_st | 0.481993156 | 0.01319139 |
| hsa-miR-520h_st | 2.47623046 | 1.940984919 |
| hsa-miR-522-star_st | 5.305630841 | 5.264098188 |
| hsa-miR-522_st | 2.478183936 | 1.895475028 |
| hsa-miR-519a-star_st | 5.184718328 | 4.708063184 |
| hsa-miR-519a_st | 1.003844498 | −0.2036258 |
| hsa-miR-527_st | 6.731023724 | 6.800021164 |
| hsa-miR-516a-5p_st | 6.692885778 | 6.527959031 |
| hsa-miR-516a-3p_st | 2.450094383 | 2.582193422 |
| hsa-miR-499-5p_st | 1.861198916 | 2.610925515 |
| hsa-miR-499-3p_st | 2.432227345 | 2.810092404 |
| hsa-miR-500_st | 5.787373445 | 6.226006184 |
| hsa-miR-500-star_st | 8.659756161 | 8.235187611 |
| hsa-miR-501-5p_st | 5.506843019 | 5.371740794 |
| hsa-miR-501-3p_st | 8.332586847 | 8.434530971 |
| hsa-miR-502-5p_st | 3.624699075 | 3.598548157 |
| hsa-miR-502-3p_st | 8.304046468 | 8.354207782 |
| hsa-miR-503_st | 10.35536084 | 10.03141346 |
| hsa-miR-504_st | 5.299415045 | 5.25691888 |
| hsa-miR-505-star_st | 7.110844003 | 6.874504071 |
| hsa-miR-505_st | 4.431591636 | 4.97862698 |
| hsa-miR-513a-5p_st | 12.57453975 | 12.40409996 |
| hsa-miR-513a-3p_st | 0.733097316 | 0.754228704 |
| hsa-miR-506_st | 3.524719744 | 3.489615805 |
| hsa-miR-507_st | 0.85212544 | 0.690231774 |
| hsa-miR-508-5p_st | 8.033954273 | 7.630762104 |
| hsa-miR-508-3p_st | −0.087132434 | 0.513031793 |
| hsa-miR-509-5p_st | 4.297004359 | 4.395609608 |
| hsa-miR-509-3p_st | 4.750143061 | 4.798983317 |
| hsa-miR-510_st | 7.718626991 | 7.464218402 |
| hsa-miR-514_st | 1.077241247 | 1.041680351 |
| hsa-miR-532-5p_st | 7.578802747 | 7.683060504 |
| hsa-miR-532-3p_st | 7.764361152 | 7.495756056 |
| hsa-miR-455-5p_st | 3.087275982 | 3.425044847 |
| hsa-miR-455-3p_st | 7.972963946 | 7.896811076 |
| hsa-miR-539_st | 3.112329336 | 3.799372521 |
| hsa-miR-544_st | 0.299770042 | 0.245484392 |
| hsa-miR-545-star_st | 1.113645529 | 1.321188245 |
| hsa-miR-545_st | 0.946917782 | 0.847954117 |
| hsa-miR-487b_st | 6.654542009 | 7.610738755 |
| hsa-miR-551a_st | 3.758352738 | 3.985920239 |
| hsa-miR-552_st | 4.199515037 | 4.433157267 |
| hsa-miR-553_st | 2.33449101 | 2.187194117 |
| hsa-miR-554_st | 4.349670096 | 4.338129697 |
| hsa-miR-92b-star_st | 14.88818352 | 14.34568728 |
| hsa-miR-92b_st | 9.036014493 | 8.886752713 |
| hsa-miR-555_st | 3.462723678 | 3.549286778 |
| hsa-miR-556-5p_st | 1.021765723 | 1.087119419 |
| hsa-miR-556-3p_st | 0.922177355 | 1.405861175 |
| hsa-miR-557_st | 12.05313471 | 11.20505749 |
| hsa-miR-558_st | 1.007626154 | 1.578448715 |
| hsa-miR-559_st | 1.480291626 | 1.851825245 |
| hsa-miR-561_st | 0.685323176 | 0.984316213 |
| hsa-miR-562_st | 1.364956829 | 1.292364779 |
| hsa-miR-563_st | 2.283937537 | 2.483367256 |
| hsa-miR-564_st | 11.91476508 | 11.61783388 |
| hsa-miR-566_st | 7.206959991 | 6.968120614 |
| hsa-miR-567_st | 3.08359803 | 2.647160748 |
| hsa-miR-568_st | 0.896970276 | 0.702526504 |
| hsa-miR-551b-star_st | 10.55462092 | 10.53013012 |
| hsa-miR-551b_st | 2.240822861 | 4.127566593 |
| hsa-miR-569_st | 2.295201557 | 2.071954881 |
| hsa-miR-570_st | 1.636829077 | 2.086717741 |
| hsa-miR-571_st | 3.76203211 | 3.97757048 |
| hsa-miR-572_st | 14.15362014 | 13.59308227 |
| hsa-miR-573_st | 0.961110161 | 0.483374673 |
| hsa-miR-574-5p_st | 7.915140727 | 7.353976228 |
| hsa-miR-574-3p_st | 9.504694233 | 9.472367599 |
| hsa-miR-575_st | 12.63366407 | 12.62164652 |
| hsa-miR-576-5p_st | 3.152949869 | 3.248353853 |
| hsa-miR-576-3p_st | 4.338131663 | 4.341276842 |
| hsa-miR-577_st | 0.469479061 | 0.900775137 |
| hsa-miR-578_st | 2.870049792 | 3.529217096 |
| hsa-miR-579_st | 1.790959284 | 2.210639291 |
| hsa-miR-580_st | 1.394668608 | 0.875418302 |
| hsa-miR-581_st | 0.187283261 | −0.173434592 |
| hsa-miR-582-5p_st | 0.877884499 | 1.53241598 |
| hsa-miR-582-3p_st | 2.596233023 | 2.803156258 |
| hsa-miR-583_st | 5.62651569 | 5.642229384 |
| hsa-miR-584_st | 7.005075512 | 7.265154665 |
| hsa-miR-585_st | 3.119003391 | 2.920976271 |
| hsa-miR-548a-3p_st | 0.985804548 | 1.041264938 |
| hsa-miR-586_st | 1.82152612 | 1.714374283 |
| hsa-miR-587_st | 2.610837187 | 2.812523842 |
| hsa-miR-548b-5p_st | 2.946869422 | 3.011193963 |
| hsa-miR-548b-3p_st | 2.016986266 | 2.528967344 |
| hsa-miR-588_st | 4.818621245 | 4.938556108 |
| hsa-miR-589_st | 3.306831528 | 3.542910019 |
| hsa-miR-589-star_st | 4.826357361 | 4.825218004 |
| hsa-miR-550_st | 11.27589533 | 11.0802969 |
| hsa-miR-550-star_st | 4.322598637 | 4.543894473 |
| hsa-miR-590-5p_st | 1.82791978 | 1.992349319 |
| hsa-miR-590-3p_st | 0.740654523 | 0.690908454 |
| hsa-miR-591_st | 2.406283077 | 2.264432045 |
| hsa-miR-592_st | 0.86580964 | 1.188523192 |
| hsa-miR-593-star_st | 8.460004942 | 8.426009482 |
| hsa-miR-593_st | 5.642477172 | 5.598938979 |
| hsa-miR-595_st | 7.152264823 | 7.012245517 |
| hsa-miR-596_st | 8.080994391 | 7.298500282 |
| hsa-miR-597_st | 1.251902643 | 1.506460357 |
| hsa-miR-598_st | 5.473052421 | 6.192344153 |
| hsa-miR-599_st | 1.258021847 | 1.59907441 |
| hsa-miR-548a-5p_st | 4.064887791 | 4.209156123 |
| hsa-miR-600_st | 4.916289299 | 5.129885648 |
| hsa-miR-601_st | 5.671311079 | 5.633315841 |
| hsa-miR-602_st | 13.65021407 | 13.05871872 |
| hsa-miR-603_st | 1.527701485 | 1.680282497 |
| hsa-miR-604_st | 8.525231223 | 8.577092671 |
| hsa-miR-605_st | 5.41566227 | 5.263864058 |
| hsa-miR-606_st | 1.678877544 | 2.000197543 |
| hsa-miR-607_st | 2.235985046 | 2.61757506 |
| hsa-miR-608_st | 9.504799581 | 9.181757238 |
| hsa-miR-609_st | 2.537991792 | 2.285395847 |
| hsa-miR-610_st | 4.615027343 | 4.971379588 |
| hsa-miR-611_st | 7.668343681 | 7.042452902 |
| hsa-miR-612_st | 11.6081787 | 11.01067274 |
| hsa-miR-613_st | 3.80957039 | 3.939463569 |
| hsa-miR-614_st | 5.403195681 | 5.572939788 |
| hsa-miR-615-5p_st | 10.75917077 | 10.21282271 |
| hsa-miR-615-3p_st | 7.132840021 | 6.574777076 |
| hsa-miR-616-star_st | 3.989652289 | 4.140479152 |
| hsa-miR-616_st | 7.541006212 | 6.947855814 |
| hsa-miR-548c-5p_st | 7.477022939 | 7.683988158 |
| hsa-miR-548c-3p_st | 0.486632096 | 0.874875836 |
| hsa-miR-617_st | 5.398827542 | 5.578629957 |
| hsa-miR-618_st | 2.643726601 | 2.945488042 |
| hsa-miR-619_st | 3.663548124 | 3.719574413 |
| hsa-miR-620_st | 2.391568642 | 2.562539354 |
| hsa-miR-621_st | 3.318204031 | 3.184787675 |
| hsa-miR-622_st | 8.546639189 | 8.722420297 |
| hsa-miR-623_st | 9.082281079 | 8.432712246 |
| hsa-miR-624-star_st | 3.687764447 | 3.576582665 |
| hsa-miR-624_st | 2.877907129 | 2.407143146 |
| hsa-miR-625_st | 6.906438656 | 6.669217579 |
| hsa-miR-625-star_st | 3.507199647 | 3.462561797 |
| hsa-miR-626_st | 1.607688443 | 2.027970356 |
| hsa-miR-627_st | 1.20076824 | 1.249802704 |
| hsa-miR-628-5p_st | 2.106074909 | 2.284612448 |
| hsa-miR-628-3p_st | 9.430978209 | 9.35461302 |
| hsa-miR-629_st | 7.418463258 | 7.728700733 |
| hsa-miR-629-star_st | 5.16556691 | 5.103179592 |
| hsa-miR-630_st | 6.33680443 | 6.265362285 |
| hsa-miR-631_st | 6.149324128 | 6.0203911 |
| hsa-miR-33b_st | 1.004003601 | 0.854850991 |
| hsa-miR-33b-star_st | 6.51120694 | 6.175282191 |
| hsa-miR-632_st | 3.681695917 | 3.876147355 |
| hsa-miR-633_st | 1.51479748 | 1.708930463 |
| hsa-miR-634_st | 5.529322114 | 5.54532548 |
| hsa-miR-635_st | 4.747459745 | 4.591006145 |
| hsa-miR-636_st | 9.379185909 | 8.596596543 |
| hsa-miR-637_st | 12.59894884 | 11.90191978 |
| hsa-miR-638_st | 16.13306369 | 15.71085502 |
| hsa-miR-639_st | 8.836316153 | 8.16788221 |
| hsa-miR-640_st | 6.577209801 | 6.328734865 |
| hsa-miR-641_st | 3.180037046 | 3.217088023 |
| hsa-miR-642_st | 3.021492034 | 3.515875701 |
| hsa-miR-643_st | 1.726001141 | 1.96185173 |
| hsa-miR-644_st | 1.989357915 | 1.915307772 |
| hsa-miR-645_st | 2.512997595 | 2.673384755 |
| hsa-miR-646_st | 3.116591723 | 3.168369747 |
| hsa-miR-647_st | 5.16631349 | 4.860732775 |
| hsa-miR-648_st | 7.615330772 | 7.266002473 |
| hsa-miR-649_st | 3.96574302 | 4.059737157 |
| hsa-miR-650_st | 10.06044403 | 10.0099794 |
| hsa-miR-651_st | 0.684569812 | 0.804540553 |
| hsa-miR-652_st | 7.597686981 | 7.477918948 |
| hsa-miR-548d-5p_st | 6.439672725 | 6.516081183 |
| hsa-miR-548d-3p_st | 1.792565954 | 2.223110919 |
| hsa-miR-661_st | 7.37367489 | 6.757648444 |
| hsa-miR-662_st | 6.131883283 | 6.393108462 |
| hsa-miR-663_st | 15.91309276 | 15.43246749 |
| hsa-miR-449b_st | 4.752576256 | 4.951235083 |
| hsa-miR-449b-star_st | 7.937832784 | 7.377443096 |
| hsa-miR-653_st | 0.719682533 | 0.911550982 |
| hsa-miR-411_st | 3.176729607 | 3.313415673 |
| hsa-miR-411-star_st | 3.760059788 | 3.880071744 |
| hsa-miR-654-5p_st | 10.78060499 | 10.30837255 |
| hsa-miR-654-3p_st | 3.620176302 | 3.884923887 |
| hsa-miR-655_st | 0.664932699 | 1.145258915 |
| hsa-miR-656_st | 2.531279986 | 2.26695859 |
| hsa-miR-549_st | 2.401667676 | 2.353313268 |
| hsa-miR-657_st | 3.89613112 | 4.123441906 |
| hsa-miR-658_st | 12.09232334 | 11.56240926 |
| hsa-miR-659_st | 10.70003607 | 10.12252061 |
| hsa-miR-660_st | 6.518421432 | 7.079439851 |
| hsa-miR-421_st | 6.00288855 | 6.630290342 |
| hsa-miR-542-5p_st | 7.483037941 | 7.486822332 |
| hsa-miR-542-3p_st | 5.370705982 | 5.929726337 |
| hsa-miR-758_st | 2.378455527 | 2.398144198 |
| hsa-miR-1264_st | 2.20962188 | 2.661817877 |
| hsa-miR-671-5p_st | 11.40166092 | 11.03723057 |
| hsa-miR-671-3p_st | 6.528072592 | 6.69575689 |
| hsa-miR-668_st | 8.411667957 | 7.779660941 |
| hsa-miR-767-5p_st | 2.712400421 | 2.837611792 |
| hsa-miR-767-3p_st | 2.279971129 | 2.521160045 |
| hsa-miR-1224-5p_st | 13.02261373 | 12.69567997 |
| hsa-miR-1224-3p_st | 5.418759666 | 5.318771817 |
| hsa-miR-320b_st | 11.53560732 | 11.18171255 |
| hsa-miR-320c_st | 10.93221694 | 10.78402919 |
| hsa-miR-1296_st | 6.626077061 | 6.369757726 |
| hsa-miR-1468_st | 5.493937398 | 5.340347087 |
| hsa-miR-1323_st | 7.652187714 | 7.155390606 |
| hsa-miR-1271_st | 6.393877363 | 6.763362299 |
| hsa-miR-1301_st | 7.676610365 | 7.777686165 |
| hsa-miR-454-star_st | 1.778710143 | 1.840510296 |
| hsa-miR-454_st | 3.84900782 | 5.161838868 |
| hsa-miR-1185_st | 0.903848628 | 0.903941085 |
| hsa-miR-449c_st | 6.490765356 | 6.179834837 |
| hsa-miR-449c-star_st | 3.93626542 | 3.464589841 |
| hsa-miR-1283_st | 2.203518171 | 2.150241028 |
| hsa-miR-769-5p_st | 6.670833939 | 7.058058874 |
| hsa-miR-769-3p_st | 9.674096826 | 9.061906854 |
| hsa-miR-766_st | 7.556645354 | 7.563965981 |
| hsa-miR-762_st | 16.05950466 | 15.61270904 |
| hsa-miR-802_st | 0.779100184 | 0.761326081 |
| hsa-miR-670_st | 4.446881202 | 4.354140447 |
| hsa-miR-1298_st | 3.691482224 | 4.017482251 |
| hsa-miR-2113_st | 5.316424475 | 5.465814895 |
| hsa-miR-761_st | 7.36539739 | 7.029608967 |
| hsa-miR-764_st | 5.695650751 | 5.327006012 |
| hsa-miR-759_st | 1.193641773 | 1.581856953 |
| hsa-miR-765_st | 8.937706312 | 8.6722512 |
| hsa-miR-770-5p_st | 6.622586426 | 6.216152999 |
| hsa-miR-675_st | 13.34487139 | 12.87896725 |
| hsa-miR-675-star_st | 7.219062545 | 6.485855347 |
| hsa-miR-298_st | 10.16448849 | 9.821490618 |
| hsa-miR-891a_st | 6.014178373 | 6.429310262 |
| hsa-miR-300_st | 5.014743946 | 4.857859675 |
| hsa-miR-886-5p_st | 12.23716753 | 11.52600293 |
| hsa-miR-886-3p_st | 10.39803241 | 10.13227778 |
| hsa-miR-892a_st | 3.783359396 | 3.765477795 |
| hsa-miR-220b_st | 3.505063834 | 3.322030411 |
| hsa-miR-450b-5p_st | 0.310395567 | 0.700205503 |
| hsa-miR-450b-3p_st | 0.789816886 | 0.838964653 |
| hsa-miR-874_st | 12.02865366 | 11.87100435 |
| hsa-miR-890_st | 5.257368933 | 6.017857157 |
| hsa-miR-891b_st | 2.691758155 | 3.435246962 |
| hsa-miR-220c_st | 5.629562702 | 5.706069794 |
| hsa-miR-888_st | 1.583771207 | 2.258563541 |
| hsa-miR-888-star_st | 1.015092215 | 0.959580777 |
| hsa-miR-892b_st | 3.683824539 | 4.265863069 |
| hsa-miR-541-star_st | 6.248172262 | 5.977820773 |
| hsa-miR-541_st | 7.998423438 | 7.571554986 |
| hsa-miR-889_st | 1.486734324 | 1.655074897 |
| hsa-miR-875-5p_st | 0.40506701 | −0.059752724 |
| hsa-miR-875-3p_st | 2.935340082 | 3.201164456 |
| hsa-miR-876-5p_st | 0.080136788 | −0.131888894 |
| hsa-miR-876-3p_st | 1.329813454 | 1.431587076 |
| hsa-miR-708_st | 6.776101765 | 6.627294578 |
| hsa-miR-708-star_st | 3.781738231 | 3.525256341 |
| hsa-miR-147b_st | 4.032586133 | 3.909008612 |
| hsa-miR-190b_st | 0.625895379 | −0.027698037 |
| hsa-miR-744_st | 14.59343027 | 14.15360767 |
| hsa-miR-744-star_st | 3.960724298 | 4.339519221 |
| hsa-miR-885-5p_st | 7.379421647 | 6.463218573 |
| hsa-miR-885-3p_st | 13.74342773 | 12.99148095 |
| hsa-miR-877_st | 11.90131515 | 11.57702037 |
| hsa-miR-877-star_st | 6.968994835 | 6.568547454 |
| hsa-miR-887_st | 8.654075735 | 8.391962876 |
| hsa-miR-665_st | 12.35824302 | 12.18147536 |
| hsa-miR-873_st | 4.316846102 | 4.24643027 |
| hsa-miR-543_st | 3.406031474 | 3.79241466 |
| hsa-miR-374b_st | 5.150898578 | 6.230702371 |
| hsa-miR-374b-star_st | 1.475215417 | 1.964696616 |
| hsa-miR-760_st | 10.58492169 | 9.909982838 |
| hsa-miR-301b_st | 0.463088948 | 1.257404871 |
| hsa-miR-216b_st | 3.554446998 | 4.066107039 |
| hsa-miR-208b_st | 2.532777991 | 2.83531643 |
| hsa-miR-920_st | 10.32386499 | 9.747609982 |
| hsa-miR-921_st | 10.78309218 | 10.49667967 |
| hsa-miR-922_st | 5.220000992 | 5.081167591 |
| hsa-miR-924_st | 2.037277431 | 2.619301274 |
| hsa-miR-509-3-5p_st | 5.964166115 | 5.646923572 |
| hsa-miR-933_st | 10.76296419 | 10.81375963 |
| hsa-miR-934_st | 3.426410172 | 3.418200873 |
| hsa-miR-935_st | 6.676731857 | 5.933023411 |
| hsa-miR-936_st | 10.24252649 | 9.963466283 |
| hsa-miR-937_st | 4.672239571 | 4.712912089 |
| hsa-miR-938_st | 4.281396098 | 4.071606338 |
| hsa-miR-939_st | 13.39670918 | 12.81677081 |
| hsa-miR-940_st | 8.227128388 | 7.95454125 |
| hsa-miR-941_st | 5.314064696 | 5.2460144 |
| hsa-miR-942_st | 1.56275362 | 1.929482676 |
| hsa-miR-943_st | 7.961964145 | 7.15536416 |
| hsa-miR-944_st | 1.454803899 | 1.5294701 |
| hsa-miR-297_st | 4.849327299 | 5.197471883 |
| hsa-miR-1178_st | 3.835439593 | 3.716716736 |
| hsa-miR-1179_st | 1.219093489 | 1.332146361 |
| hsa-miR-1180_st | 9.654183445 | 9.165207337 |
| hsa-miR-1181_st | 9.912039472 | 9.358092976 |
| hsa-miR-1182_st | 10.72063329 | 10.26309024 |
| hsa-miR-1183_st | 10.56574562 | 10.21442752 |
| hsa-miR-1184_st | 4.06854417 | 3.841068542 |
| hsa-miR-1225-5p_st | 13.99463783 | 13.60744016 |
| hsa-miR-1225-3p_st | 8.023302948 | 7.415903622 |
| hsa-miR-1226-star_st | 11.02379322 | 10.62722384 |
| hsa-miR-1226_st | 5.87284576 | 5.708668387 |
| hsa-miR-1227_st | 5.47922344 | 5.087087304 |
| hsa-miR-1228-star_st | 16.00703846 | 15.55054414 |
| hsa-miR-1228_st | 8.183898455 | 7.754592554 |
| hsa-miR-1229_st | 7.8660255 | 7.425379155 |
| hsa-miR-1231_st | 13.99160279 | 13.29293761 |
| hsa-miR-1233_st | 7.140881909 | 6.813134409 |
| hsa-miR-1234_st | 6.978386089 | 6.703978411 |
| hsa-miR-1236_st | 5.757283186 | 5.562027912 |
| hsa-miR-1237_st | 8.049528369 | 7.501759124 |
| hsa-miR-1238_st | 6.929779579 | 6.674332143 |
| hsa-miR-1200_st | 5.018956665 | 4.660480029 |
| hsa-miR-1201_st | 7.83962411 | 8.60963805 |
| hsa-miR-1202_st | 13.13333492 | 12.59373454 |
| hsa-miR-1203_st | 8.407178357 | 8.308694904 |
| hsa-miR-663b_st | 10.18073743 | 10.10219451 |
| hsa-miR-1204_st | 5.038031282 | 4.657796701 |
| hsa-miR-1205_st | 3.414346931 | 3.533583038 |
| hsa-miR-1206_st | 0.654108253 | 1.265207014 |
| hsa-miR-1207-5p_st | 15.09383225 | 14.67994122 |
| hsa-miR-1207-3p_st | 3.967191169 | 3.376750907 |
| hsa-miR-1208_st | 8.547871045 | 7.850096533 |
| hsa-miR-548e_st | 0.473225938 | 0.815593405 |
| hsa-miR-548j_st | 3.359555166 | 2.980872631 |
| hsa-miR-1285_st | 12.97329191 | 12.86031885 |
| hsa-miR-1286_st | 8.330094451 | 8.000560127 |
| hsa-miR-1287_st | 5.796143688 | 5.951267014 |
| hsa-miR-1289_st | 4.644854383 | 4.753867218 |
| hsa-miR-1290_st | 9.369553951 | 9.015379911 |
| hsa-miR-1291_st | 8.258068261 | 8.11301893 |
| hsa-miR-548k_st | 2.172935385 | 2.681926565 |
| hsa-miR-1293_st | 7.784448988 | 7.443396866 |
| hsa-miR-1294_st | 5.615706602 | 5.430705677 |
| hsa-miR-1295_st | 5.72171546 | 5.634436005 |
| hsa-miR-1297_st | 0.694687517 | 1.273957483 |
| hsa-miR-1299_st | 5.362501545 | 5.034147137 |
| hsa-miR-5481_st | 4.090651354 | 3.99206717 |
| hsa-miR-1302_st | 1.757901869 | 1.705103066 |
| hsa-miR-1303_st | 10.5092456 | 10.16222173 |
| hsa-miR-1304_st | 6.383188311 | 6.731791893 |
| hsa-miR-1305_st | 2.218622047 | 2.295353425 |
| hsa-miR-1243_st | 3.115792724 | 2.908600169 |
| hsa-miR-548f_st | 0.934036318 | 0.786744918 |
| hsa-miR-1244_st | 8.089934765 | 8.607202837 |
| hsa-miR-1245_st | 3.227053374 | 3.230305643 |
| hsa-miR-1246_st | 13.26254202 | 13.10299414 |
| hsa-miR-1247_st | 6.519744424 | 6.19193942 |
| hsa-miR-1248_st | 9.571458845 | 9.931793782 |
| hsa-miR-1249_st | 4.697464396 | 4.741051931 |
| hsa-miR-1250_st | 8.601722913 | 8.265194057 |
| hsa-miR-1251_st | 4.332141423 | 4.845112511 |
| hsa-miR-1253_st | 4.352494892 | 4.197114342 |
| hsa-miR-1254_st | 11.07447655 | 10.84067505 |
| hsa-miR-1255a_st | 4.71253267 | 4.900448328 |
| hsa-miR-1256_st | 2.921954678 | 3.257665828 |
| hsa-miR-1257_st | 3.738524139 | 3.862628094 |
| hsa-miR-1258_st | 3.980487676 | 4.024276093 |
| hsa-miR-1259_st | 1.793876877 | 2.134496387 |
| hsa-miR-1260_st | 10.51371892 | 10.52978062 |
| hsa-miR-548g_st | 0.427863344 | 0.639394206 |
| hsa-miR-1261_st | 7.500943582 | 7.388669793 |
| hsa-miR-1262_st | 2.385426572 | 2.18526919 |
| hsa-miR-1263_st | 3.328855641 | 2.977757046 |
| hsa-miR-548n_st | 1.476271083 | 1.519100638 |
| hsa-miR-548m_st | 1.986854867 | 2.284258132 |
| hsa-miR-1265_st | 4.836064174 | 4.666927642 |
| hsa-miR-548o_st | 1.719377422 | 1.288016821 |
| hsa-miR-1266_st | 8.713590792 | 8.222220746 |
| hsa-miR-1267_st | 3.855578054 | 3.480577888 |
| hsa-miR-1268_st | 15.51819611 | 15.04306878 |
| hsa-miR-1269_st | 4.925081179 | 4.860540477 |
| hsa-miR-1270_st | 6.2456038 | 6.119878209 |
| hsa-miR-1272_st | 3.323982487 | 3.299811978 |
| hsa-miR-1273_st | 10.89348275 | 10.8013459 |
| hsa-miR-1274a_st | 9.866616109 | 10.95409808 |
| hsa-miR-548h_st | 2.708976669 | 3.075521318 |
| hsa-miR-1275_st | 14.04621179 | 13.66733549 |
| hsa-miR-1276_st | 5.91403493 | 5.705786102 |
| hsa-miR-302e_st | 2.775251808 | 2.420822294 |
| hsa-miR-302f_st | 1.051179195 | 1.454558008 |
| hsa-miR-1277_st | 1.921769944 | 1.875581452 |
| hsa-miR-548p_st | 1.26545941 | 1.412701566 |
| hsa-miR-548i_st | 4.337002107 | 4.422845234 |
| hsa-miR-1278_st | 3.28235357 | 3.422861193 |
| hsa-miR-1279_st | 2.655743736 | 3.153072811 |
| hsa-miR-1274b_st | 13.40685468 | 14.05745456 |
| hsa-miR-1281_st | 9.162432854 | 8.628380901 |
| hsa-miR-1282_st | 3.720097483 | 4.002137534 |
| hsa-miR-1284_st | 3.906481147 | 3.839660908 |
| hsa-miR-1288_st | 4.057295362 | 4.309374769 |
| hsa-miR-1292_st | 12.54990676 | 12.12104333 |
| hsa-miR-1252_st | 1.390910014 | 1.641722688 |
| hsa-miR-1255b_st | 7.861905402 | 8.2114196 |
| hsa-miR-1280_st | 14.99196599 | 14.94288127 |
| hsa-miR-1308_st | 14.96349933 | 15.13166088 |
| hsa-miR-664-star_st | 9.899383779 | 9.542708113 |
| hsa-miR-664_st | 5.466549309 | 5.260588583 |
| hsa-miR-1306_st | 6.8937086 | 6.604984224 |
| hsa-miR-1307_st | 13.98022262 | 13.37529099 |
| hsa-miR-513b_st | 10.57485724 | 10.45748832 |
| hsa-miR-513c_st | 8.74116087 | 8.349383978 |
| hsa-miR-1321_st | 7.503803552 | 7.073330049 |
| hsa-miR-1322_st | 3.669120279 | 3.556889047 |
| hsa-miR-720_st | 15.04724175 | 15.07056161 |
| hsa-miR-1197_st | 1.963900545 | 2.054211227 |
| hsa-miR-1324_st | 1.455355935 | 1.888812334 |
| hsa-miR-1469_st | 16.0183398 | 15.54754242 |
| hsa-miR-1470_st | 5.754747808 | 5.158367754 |
| hsa-miR-1471_st | 10.86730917 | 10.48569035 |
| hsa-miR-1537_st | 1.182421653 | 1.412645392 |
| hsa-miR-1538_st | 7.788283814 | 7.622768161 |
| hsa-miR-1539_st | 5.970294118 | 5.973367867 |
| hsa-miR-103-as_st | 2.508175856 | 2.266885141 |
| hsa-miR-320d_st | 9.717392149 | 9.756189985 |
| hsa-miR-1825_st | 6.7812821 | 6.397975378 |
| hsa-miR-1826_st | 15.95940582 | 15.68532265 |
| hsa-miR-1827_st | 4.494451222 | 5.671983153 |
| hsa-miR-1908_st | 16.09437218 | 15.6685715 |
| hsa-miR-1909-star_st | 9.074029033 | 8.420395636 |
| hsa-miR-1909_st | 14.82498487 | 14.31704517 |
| hsa-miR-1910_st | 10.19701023 | 9.165819315 |
| hsa-miR-1911_st | 3.452208679 | 3.530335335 |
| hsa-miR-1911-star_st | 4.838918994 | 4.939768155 |
| hsa-miR-1912_st | 3.889736968 | 4.001458712 |
| hsa-miR-1913_st | 8.208934885 | 7.816539543 |
| hsa-miR-1914_st | 6.948960342 | 6.641912822 |
| hsa-miR-1914-star_st | 12.76141618 | 12.28137688 |
| hsa-miR-1915-star_st | 7.880688641 | 7.371510327 |
| hsa-miR-1915_st | 16.09441936 | 15.65666258 |
| hsa-miR-1972_st | 13.28967445 | 13.0889173 |
| hsa-miR-1973_st | 14.46489111 | 14.71345884 |
| hsa-miR-1975_st | 15.83144955 | 15.609638 |
| hsa-miR-1976_st | 7.955652531 | 7.324338717 |
| hsa-miR-1979_st | 15.17466845 | 14.92056909 |
| hsa-miR-2052_st | 0.261150813 | 0.879065556 |
| hsa-miR-2053_st | 1.566913167 | 1.772546409 |
| hsa-miR-2054_st | −0.371432679 | −0.054846939 |
| hsa-miR-2110_st | 10.7596285 | 10.32133115 |
| hsa-miR-2114_st | 2.275045318 | 2.652793175 |
| hsa-miR-2114-star_st | 5.893322871 | 6.090650086 |
| hsa-miR-2115_st | 1.669248276 | 2.025263831 |
| hsa-miR-2115-star_st | 7.86781663 | 7.517753205 |
| hsa-miR-2116_st | 5.382899873 | 5.301283894 |
| hsa-miR-2116-star_st | 4.313940938 | 4.570962705 |
| hsa-miR-2117_st | 1.989389206 | 1.81859151 |
| hsa-miR-548q_st | 7.909367287 | 7.135060897 |
| hsa-miR-2276_st | 10.04754803 | 9.612458781 |
| hsa-miR-2277_st | 8.843380123 | 8.242791768 |
| hsa-miR-2278_st | 5.46905257 | 5.172606924 |
| hsa-miR-711_st | 11.50174419 | 10.96229128 |
| hsa-miR-718_st | 7.442014014 | 7.163231535 |
| hsa-miR-2861_st | 16.09867509 | 15.67854395 |
| hsa-miR-2909_st | 2.843929561 | 3.059059612 |
| hsa-miR-3115_st | 1.737701734 | 1.895906102 |
| hsa-miR-3116_st | 5.846455429 | 5.994577546 |
| hsa-miR-3117_st | 2.792558003 | 2.7665622 |
| hsa-miR-3118_st | 1.966621914 | 2.355950452 |
| hsa-miR-3119_st | 1.064257222 | 0.979500777 |
| hsa-miR-3120_st | 5.873825771 | 5.522932348 |
| hsa-miR-3121_st | 6.72575369 | 6.585337578 |
| hsa-miR-3122_st | 11.19876638 | 10.60586491 |
| hsa-miR-3123_st | 0.811165914 | −0.188615176 |
| hsa-miR-3124_st | 11.71964185 | 11.16453936 |
| hsa-miR-548s_st | 1.466493177 | 1.689971077 |
| hsa-miR-3125_st | 6.876702817 | 6.818744686 |
| hsa-miR-3126-5p_st | 11.6003181 | 11.27378363 |
| hsa-miR-3126-3p_st | 4.320522288 | 4.289572758 |
| hsa-miR-3127_st | 9.124840533 | 8.868822515 |
| hsa-miR-3128_st | 2.735917851 | 2.578431728 |
| hsa-miR-3129_st | 3.581165201 | 3.371565647 |
| hsa-miR-3130-5p_st | 6.60574064 | 6.353294536 |
| hsa-miR-3130-3p_st | 7.726838449 | 7.18848278 |
| hsa-miR-3131_st | 11.91732799 | 11.41437778 |
| hsa-miR-3132_st | 8.733534161 | 8.548941914 |
| hsa-miR-3133_st | 2.379308925 | 2.704068889 |
| hsa-miR-378b_st | 8.681317348 | 8.650627772 |
| hsa-miR-3134_st | 1.486292562 | 1.585026115 |
| hsa-miR-3135_st | 6.388081763 | 5.945197855 |
| hsa-miR-466_st | 5.570854799 | 5.542584775 |
| hsa-miR-3136_st | 6.025449661 | 5.835307058 |
| hsa-miR-544b_st | 2.395651967 | 2.329439708 |
| hsa-miR-3137_st | 10.77465477 | 10.2263542 |
| hsa-miR-3138_st | 10.68211816 | 10.28813745 |
| hsa-miR-3139_st | 2.392149157 | 2.319510504 |
| hsa-miR-3140_st | 4.519529715 | 4.369874672 |
| hsa-miR-548t_st | 2.16050833 | 2.590748278 |
| hsa-miR-3141_st | 15.23935882 | 14.69454712 |
| hsa-miR-3142_st | 1.230043669 | 1.403627739 |
| hsa-miR-3143_st | 0.40032393 | 0.585435203 |
| hsa-miR-548u_st | 1.153531581 | 1.827011911 |
| hsa-miR-3144-5p_st | 4.500348126 | 4.861469455 |
| hsa-miR-3144-3p_st | 2.606286299 | 2.956528592 |
| hsa-miR-3145_st | 0.093993449 | 0.518199564 |
| hsa-miR-1273c_st | 10.04905189 | 9.856293901 |
| hsa-miR-3146_st | 3.4426515 | 3.404563434 |
| hsa-miR-3147_st | 11.26421121 | 10.85330475 |
| hsa-miR-548v_st | 2.734247475 | 3.018309976 |
| hsa-miR-3148_st | 4.747538073 | 4.463907972 |
| hsa-miR-3149_st | 2.666562564 | 2.768578604 |
| hsa-miR-3150_st | 9.397553648 | 8.840212022 |
| hsa-miR-3151_st | 11.32817214 | 10.69238559 |
| hsa-miR-3152_st | 6.838886942 | 6.526417665 |
| hsa-miR-3153_st | 9.393915286 | 8.916847838 |
| hsa-miR-3074_st | 4.493265824 | 4.089405742 |
| hsa-miR-3154_st | 11.43417874 | 11.02236951 |
| hsa-miR-3155_st | 6.870088743 | 6.714149239 |
| hsa-miR-3156_st | 9.93990287 | 10.0849857 |
| hsa-miR-3157_st | 5.38163833 | 5.094757246 |
| hsa-miR-3158_st | 4.236335397 | 4.274469069 |
| hsa-miR-3159_st | 4.040287102 | 3.801415135 |
| hsa-miR-3160_st | 5.161150506 | 5.378709596 |
| hsa-miR-3161_st | 6.51169066 | 6.557791334 |
| hsa-miR-3162_st | 13.59267667 | 13.14054899 |
| hsa-miR-3163_st | 7.645100343 | 7.48987569 |
| hsa-miR-3164_st | 6.163469175 | 6.189816592 |
| hsa-miR-3165_st | 6.882731374 | 6.535597214 |
| hsa-miR-3166_st | 3.973883057 | 4.118281598 |
| hsa-miR-1260b_st | 13.93656531 | 13.99271742 |
| hsa-miR-3167_st | 1.377189008 | 1.42325091 |
| hsa-miR-3168_st | 2.194258898 | 2.15096857 |
| hsa-miR-3169_st | 3.70478395 | 3.682058672 |
| hsa-miR-3170_st | 6.064431816 | 5.836019309 |
| hsa-miR-3171_st | 2.352321358 | 2.025211561 |
| hsa-miR-3172_st | 15.23599263 | 15.04216648 |
| hsa-miR-3173_st | 9.032862067 | 8.749245336 |
| hsa-miR-1193_st | 9.830435489 | 9.725716769 |
| hsa-miR-323b-5p_st | 7.434430581 | 7.205363706 |
| hsa-miR-323b-3p_st | 2.357569768 | 2.866142211 |
| hsa-miR-3174_st | 6.173342148 | 6.173925415 |
| hsa-miR-3175_st | 12.95023052 | 12.79495123 |
| hsa-miR-3176_st | 10.61297987 | 10.65372575 |
| hsa-miR-3177_st | 11.49185794 | 11.07616235 |
| hsa-miR-3178_st | 15.64109228 | 15.24205841 |
| hsa-miR-3179_st | 3.289533778 | 2.950144271 |
| hsa-miR-3180-5p_st | 7.725233167 | 7.083215398 |
| hsa-miR-3180-3p_st | 14.81867448 | 14.0986637 |
| hsa-miR-548w_st | 6.223862497 | 6.001907668 |
| hsa-miR-3181_st | 8.412913395 | 7.93524875 |
| hsa-miR-3182_st | 2.946246872 | 3.014694646 |
| hsa-miR-3183_st | 3.775060955 | 3.752226002 |
| hsa-miR-3184_st | 9.50508687 | 9.123334445 |
| hsa-miR-3185_st | 15.22528591 | 14.55369878 |
| hsa-miR-3065-5p_st | 2.925847678 | 4.183884524 |
| hsa-miR-3065-3p_st | 3.813962114 | 4.624189553 |
| hsa-miR-3186-5p_st | 4.019883608 | 4.156746826 |
| hsa-miR-3186-3p_st | 8.641386086 | 8.360382996 |
| hsa-miR-3187_st | 12.62991206 | 11.97054122 |
| hsa-miR-3188_st | 12.96496319 | 12.37641503 |
| hsa-miR-3189_st | 7.393244154 | 6.98199517 |
| hsa-miR-320c_st | 9.526714554 | 9.465066146 |
| hsa-miR-3190-5p_st | 9.796876407 | 9.541241883 |
| hsa-miR-3190-3p_st | 10.01846168 | 9.754508553 |
| hsa-miR-3191_st | 11.40731033 | 11.18992752 |
| hsa-miR-3192_st | 8.679024367 | 8.447355999 |
| hsa-miR-3193_st | 5.958950748 | 5.926624483 |
| hsa-miR-3194_st | 10.4157656 | 10.01084942 |
| hsa-miR-3195_st | 12.82491936 | 12.24024715 |
| hsa-miR-3196_st | 16.13437134 | 15.7220917 |
| hsa-miR-548x_st | 0.378130116 | 0.540103906 |
| hsa-miR-3197_st | 15.02973494 | 14.78116356 |
| hsa-miR-3198_st | 9.632039821 | 9.076169918 |
| hsa-miR-3199_st | 4.754752368 | 4.608359402 |
| hsa-miR-3200_st | 3.154084708 | 3.872455472 |
| hsa-miR-3201_st | 3.877606697 | 3.458973914 |
| hsa-miR-514b-5p_st | 9.874268622 | 9.500407674 |
| hsa-miR-514b-3p_st | 1.402356844 | 1.777999516 |
| hsa-miR-3202_st | 7.738810921 | 7.517156041 |
| hsa-miR-1273d_st | 11.60280398 | 11.35626331 |
| hsa-miR-4295_st | 1.931903283 | 2.132044697 |
| hsa-miR-4296_st | 7.275021063 | 6.644864124 |
| hsa-miR-4297_st | 5.343286852 | 5.422587806 |
| hsa-miR-378c_st | 8.420518509 | 8.562011567 |
| hsa-miR-4293_st | 4.242231179 | 4.368393525 |
| hsa-miR-4294_st | 7.650123448 | 7.374027486 |
| hsa-miR-4301_st | 2.228095075 | 2.133986309 |
| hsa-miR-4299_st | 12.53474584 | 12.73079626 |
| hsa-miR-4298_st | 13.00339819 | 12.74731493 |
| hsa-miR-4300_st | 7.580250098 | 7.088464462 |
| hsa-miR-4304_st | 5.967406714 | 5.875109351 |
| hsa-miR-4302_st | 5.276100863 | 5.236412449 |
| hsa-miR-4303_st | 5.807861351 | 6.002325091 |
| hsa-miR-4305_st | 3.61704062 | 3.376297467 |
| hsa-miR-4306_st | 9.43700333 | 9.385309266 |
| hsa-miR-4309_st | 3.43346671 | 3.560615807 |
| hsa-miR-4307_st | 2.384760497 | 2.711173548 |
| hsa-miR-4308_st | 1.416878319 | 2.010464942 |
| hsa-miR-4310_st | 3.052380358 | 3.628266963 |
| hsa-miR-4311_st | 4.892451406 | 4.608785295 |
| hsa-miR-4312_st | 6.472716919 | 6.127945376 |
| hsa-miR-4313_st | 5.69343871 | 5.40524793 |
| hsa-miR-4315_st | 2.677150824 | 3.115022119 |
| hsa-miR-4316_st | 7.507516492 | 7.101350428 |
| hsa-miR-4314_st | 8.52483982 | 8.259941138 |
| hsa-miR-4318_st | 3.024705032 | 2.975681662 |
| hsa-miR-4319_st | 3.396904748 | 3.216724224 |
| hsa-miR-4320_st | 2.743820827 | 2.628626923 |
| hsa-miR-4317_st | 8.854663529 | 9.049254113 |
| hsa-miR-4322_st | 12.59542136 | 11.92560502 |
| hsa-miR-4321_st | 10.11377839 | 9.267081291 |
| hsa-miR-4323_st | 5.591073958 | 5.409930688 |
| hsa-miR-4324_st | 7.0470127 | 7.291965127 |
| hsa-miR-4256_st | 3.867241681 | 3.602914193 |
| hsa-miR-4257_st | 12.13785011 | 11.66505888 |
| hsa-miR-4258_st | 7.676362685 | 7.131231992 |
| hsa-miR-4259_st | 11.0180055 | 10.48497079 |
| hsa-miR-4260_st | 10.45670775 | 10.14781802 |
| hsa-miR-4253_st | 11.25302284 | 10.58511018 |
| hsa-miR-4251_st | 5.764280017 | 5.633677603 |
| hsa-miR-4254_st | 5.380678028 | 5.336710632 |
| hsa-miR-4255_st | 2.649903696 | 2.629073433 |
| hsa-miR-4252_st | 4.691837687 | 4.634410546 |
| hsa-miR-4325_st | 2.879980251 | 3.145386356 |
| hsa-miR-4326_st | 4.777538434 | 4.784173474 |
| hsa-miR-4327_st | 12.09295135 | 11.45109662 |
| hsa-miR-4261_st | 8.362010242 | 8.394140492 |
| hsa-miR-4265_st | 7.336890943 | 7.191907851 |
| hsa-miR-4266_st | 6.149713022 | 5.478919472 |
| hsa-miR-4267_st | 5.505171634 | 5.506853671 |
| hsa-miR-4262_st | 1.430263669 | 1.580298001 |
| hsa-miR-2355_st | 2.090169511 | 2.401306329 |
| hsa-miR-4268_st | 3.823106966 | 3.571521985 |
| hsa-miR-4269_st | 7.704540144 | 7.526481382 |
| hsa-miR-4263_st | 2.251916983 | 2.668325453 |
| hsa-miR-4264_st | 2.158632979 | 2.333748508 |
| hsa-miR-4270_st | 15.40441472 | 14.90180799 |
| hsa-miR-4271_st | 11.64139121 | 11.19518471 |
| hsa-miR-4272_st | 1.39695707 | 1.230182865 |
| hsa-miR-4273_st | 1.051451991 | 1.180435309 |
| hsa-miR-4276_st | 1.051009386 | 1.326833075 |
| hsa-miR-4275_st | 1.871985847 | 1.739026976 |
| hsa-miR-4274_st | 5.629978633 | 5.400048437 |
| hsa-miR-4281_st | 15.80337411 | 15.28735574 |
| hsa-miR-4277_st | 2.395172897 | 2.321021436 |
| hsa-miR-4279_st | 5.633387454 | 4.741221052 |
| hsa-miR-4278_st | 6.858792003 | 6.223618133 |
| hsa-miR-4280_st | 4.156896034 | 4.29928346 |
| hsa-miR-4282_st | 0.017248915 | 0.171181809 |
| hsa-miR-4285_st | 11.80795524 | 11.15972937 |
| hsa-miR-4283_st | 8.706335149 | 7.951747816 |
| hsa-miR-4284_st | 14.76494449 | 15.07724784 |
| hsa-miR-4286_st | 13.79575921 | 14.1297145 |
| hsa-miR-4287_st | 4.730787189 | 5.059402816 |
| hsa-miR-4288_st | 3.914839643 | 4.932580056 |
| hsa-miR-4292_st | 7.176353439 | 6.781422755 |
| hsa-miR-4289_st | 5.477452762 | 6.232237948 |
| hsa-miR-4290_st | 6.705661946 | 6.023062657 |
| hsa-miR-4291_st | 5.570408916 | 6.484801604 |
| hsa-miR-4329_st | 5.828255767 | 5.705003789 |
| hsa-miR-4330_st | 5.504211012 | 5.122778314 |
| hsa-miR-500b_st | 3.851837359 | 4.171247331 |
| hsa-miR-4328_st | 2.880115886 | 3.102092043 |

TABLE 4D
normalized array data for the 55 benign and malignant FFPE samples
(all MiRNA are from Homo sapiens)

| | LogRatio | | Se- | | SEQ |
|---|---|---|---|---|---|
| | (malignant | | quence | | ID |
| miRNA | vs benign) | Alignments | Length | Sequence | NO |
| hsa-let-7a_st | −0.048023964 | | 22 | UGAGGUAGUAGGUUGUAUAGUU | 65 |
| hsa-let-7a- | 0.217445459 | | 21 | CUAUACAAUCUACUGUCUUUC | 66 |
| star_st | | | | | |
| hsa-let-7a-2- | −0.03760635 | | 22 | CUGUACAGCCUCCUAGCUUUCC | 67 |
| star_st | | | | | |
| hsa-let-7b_st | 0.013698867 | 22:46509566-46509648 (+) | 22 | UGAGGUAGUAGGUUGUGUGGUU | 68 |
| hsa-let-7b- | −0.748341751 | 22:46509566-46509648 (+) | 22 | CUAUACAACCUACUGCCUUCCC | 69 |
| star_st | | | | | |
| hsa-let-7c_st | −0.071563315 | 21:17912148-17912231 (+) | 22 | UGAGGUAGUAGGUUGUAUGGUU | 70 |
| hsa-let-7c- | 0.127967031 | 21:17912148-17932231 (+) | 22 | UAGAGUUACACCCUGGGAGUUA | 71 |
| star_st | | | | | |
| hsa-let-7d_st | 0.083560357 | 9:96941116-96941202 (+) | 22 | AGAGGUAGUAGGUUGCAUAGUU | 72 |
| hsa-let-7d- | −0.612452206 | 9:96941116-96941202 (+) | 22 | CUAUACGACCUGCUGCCUUUCU | 73 |
| star_st | | | | | |
| hsa-let-7e_st | 0.2865631 | 19:52196039-52196117 (+) | 22 | UGAGGUAGGAGGUUGUAUAGUU | 74 |
| hsa-let-7e- | −0.214645405 | 19:52196039-52196117 (+) | 22 | CUAUACGGCCUCCUAGCUUUCC | 75 |
| star_st | | | | | |
| hsa-let-7f_st | 0.025135204 | | 22 | UGAGGUAGUAGAUUGUAUAGUU | 76 |
| hsa-let-7f-1- | −0.298460339 | | 22 | CUAUACAAUCUAUUGCCUUCCC | 77 |
| star_st | | | | | |
| hsa-let-7f-2- | 0.473970042 | | 22 | CUAUACAGUCUACUGUCUUUCC | 78 |
| star_st | | | | | |
| hsa-miR-15a_st | 1.108769019 | 13:50623255-50623337 (−) | 22 | UAGCAGCACAUAAUGGUUUGUG | 79 |
| hsa-miR-15a- | 0.167761859 | 13:50623255-50623337 (−) | 22 | CAGGCCAUAUUGUGCUGCCUCA | 80 |
| star_st | | | | | |
| hsa-miR-16_st | 0.734813434 | | 22 | UAGCAGCACGUAAAUAUUGGCG | 81 |
| hsa-miR-16-1- | 0.741299295 | | 22 | CCAGUAUUAACUGUGCUGCUGA | 82 |
| star_st | | | | | |
| hsa-miR-17_st | 0.805305178 | 13:92002859-92002942 (+) | 23 | CAAAGUGCUUACAGUGCAGGUAG | 83 |
| hsa-miR-17- | 0.414616826 | 13:92002859-92002942 (+) | 22 | ACUGCAGUGAAGGCACUUGUAG | 84 |
| star_st | | | | | |
| hsa-miR-18a_st | 0.891730358 | 13:92003005-92003075 (+) | 23 | UAAGGUGCAUCUAGUGCAGAUAG | 85 |
| hsa-miR-18a- | 0.089261898 | 13:92003005-92003075 (+) | 23 | ACUGCCCUAAGUGCUCCUUCUGG | 86 |
| star_st | | | | | |
| hsa-miR-19a- | −0.152941809 | 13:92003145-92003226 (+) | 22 | AGUUUUGCAUAGUUGCACUACA | 87 |
| star_st | | | | | |
| hsa-miR-19a_st | 0.694829137 | 13:92003145-92003226 (+) | 23 | UGUGCAAAUCUAUGCAAAACUGA | 88 |
| hsa-miR-19b-1- | 0.170383606 | | 23 | AGUUUUGCAGGUUUGCAUCCAGC | 89 |
| star_st | | | | | |
| hsa-miR-19b_st | 0.682682286 | | 23 | UGUGCAAAUCCAUGCAAAACUGA | 90 |
| hsa-miR-19b-2- | 0.118943559 | | 22 | AGUUUUGCAGGUUUGCAUUUCA | 91 |
| star_st | | | | | |
| hsa-miR-20a_st | 0.694990809 | 13:92003319-92003389 (+) | 23 | UAAAGUGCUUAUAGUGCAGGUAG | 92 |
| hsa-miR-20a- | −0.249127844 | 13:92003319-92003389 (+) | 22 | ACUGCAUUAUGAGCACUUAAAG | 93 |
| star_st | | | | | |
| hsa-miR-21_st | 2.173328901 | 17:57918627-57918698 (+) | 22 | UAGCUUAUCAGACUGAUGUUGA | 94 |
| hsa-miR-21- | 0.913325389 | 17:57918627-57918698 (+) | 21 | CAACACCAGUCGAUGGGCUGU | 95 |
| star_st | | | | | |
| hsa-miR-22- | 0.805013409 | 17:1617197-1617281 (−) | 22 | AGUUCUUCAGUGGCAAGCUUUA | 96 |
| star_st | | | | | |
| hsa-miR-22_st | 0.497050849 | 17:16I7197-1617281 (−) | 22 | AAGCUGCCAGUUGAAGAACUGU | 97 |
| hsa-miR-23a- | −0.40320726 | 19:13947401-13947473 (−) | 22 | GGGGUUCCUGGGGAUGGGAUUU | 98 |
| star_st | | | | | |
| hsa-miR-23a_st | 0.598156224 | 19:13947401-13947473 (−) | 21 | AUCACAUUGCCAGGGAUUUCC | 99 |
| hsa-miR-24-1- | 1.152267111 | | 22 | UGCCUACUGAGCUGAUAUCAGU | 100 |
| star_st | | | | | |
| hsa-miR-24_st | 0.879752178 | | 22 | UGGCUCAGUUCAGCAGGAACAG | 101 |
| hsa-miR-24-2- | 1.542735664 | | 22 | UGCCUACUGAGCUGAAACACAG | 102 |
| star_st | | | | | |
| hsa-miR-25- | −0.649750992 | 7:99691183-99691266 (−) | 21 | AGGCGGAGACUUGGGCAAUUG | 103 |
| star_st | | | | | |
| hsa-miR-25_st | 0.299753561 | 7:99691183-99691266 (−) | 22 | CAUUGCACUUGUCUCGGUCUGA | 104 |
| hsa-miR-26a_st | −0.124819372 | | 22 | UUCAAGUAAUCCAGGAUAGGCU | 105 |
| hsa-miR-26a-1- | −0.401191655 | | 22 | CCUAUUCUUGGUUACUUGCACG | 106 |
| star_st | | | | | |
| hsa-miR-26b_st | 0.231155035 | 2:219267369-219267445 (+) | 21 | UUCAAGUAAUUCAGGAUAGGU | 107 |
| hsa-miR-26b- | 0.215482208 | 2:219267369-219267445 (+) | 22 | CCUGUUCUCCAUUACUUGGCUC | 108 |
| star_st | | | | | |
| hsa-miR-27a- | 0.320877233 | 19:13947254-13947331 (−) | 22 | AGGGCUUAGCUGCUUGUGAGCA | 109 |
| star_st | | | | | |
| hsa-miR-27a_st | 1.309760051 | 19:13947254-13947331 (−) | 21 | UUCACAGUGGCUAAGUUCCGC | 110 |
| hsa-miR-28- | 0.736552791 | 3:188406569-188406654 (+) | 22 | AAGGAGCUCACAGUCUAUUGAG | 111 |
| 5p_st | | | | | |
| hsa-miR-28- | 0.469314854 | 3:188406569-188406654 (+) | 22 | CACUAGAUUGUGAGCUCCUGGA | 112 |
| 3p_st | | | | | |
| hsa-miR-29a- | 0.266553866 | 7:130561506-130561569 (−) | 22 | ACUGAUUUCUUUUGGUGUUCAG | 113 |
| star_st | | | | | |
| hsa-miR-29a_st | 0.741466995 | 7:130561506-130561569 (−) | 22 | UAGCACCAUCUGAAAUCGGUUA | 114 |
| hsa-miR-30a_st | 0.080469489 | 6:72113254-72113324 (−) | 22 | UGUAAACAUCCUCGACUGGAAG | 115 |
| hsa-miR-30a- | −1.10736252 | 6:72113254-72113324 (−) | 22 | CUUUCAGUCGGAUGUUUGCAGC | 116 |
| star_st | | | | | |
| hsa-miR-31_st | 1.622439287 | 9:21512114-21512184 (−) | 21 | AGGCAAGAUGCUGGCAUAGCU | 117 |
| hsa-miR-31- | 2.390305726 | 9:21512114-21512184 (−) | 22 | UGCUAUGCCAACAUAUUGCCAU | 118 |
| star_st | | | | | |
| hsa-miR-32_st | 0.62671376 | 9:111808509-111808578 (−) | 22 | UAUUGCACAUUACUAAGUUGCA | 119 |
| hsa-miR-32- | −0.194057854 | 9:111808509-111808578 (−) | 22 | CAAUUUAGUGUGUGUGAUAUUU | 120 |
| star_st | | | | | |
| hsa-miR-33a_st | 0.313788559 | 22:42296948-42297016 (+) | 21 | GUGCAUUGUAGUUGCAUUGCA | 121 |
| hsa-miR-33a- | 0.921600085 | 22:42296948-42297016 (+) | 22 | CAAUGUUUCCACAGUGCAUCAC | 122 |
| star_st | | | | | |
| hsa-miR-92a-1- | −0.581599889 | | 23 | AGGUUGGGAUCGGUUGCAAUGCU | 123 |
| star_st | | | | | |
| hsa-miR-92a_st | 0.144284845 | | 22 | UAUUGCACUUGUCCCGGCCUGU | 124 |
| hsa-miR-92a-2- | −0.597060048 | | 22 | GGGUGGGGAUUUGUUGCAUUAC | 125 |
| star_st | | | | | |
| hsa-miR-93_st | 0.804315226 | 7:99691391-99691470 (−) | 23 | CAAAGUGCUGUUCGUGCAGGUAG | 126 |
| hsa-miR-93- | 0.596630167 | 7:99691391-99691470 (−) | 22 | ACUGCUGAGCUAGCACUUCCCG | 127 |
| star_st | | | | | |
| hsa-miR-95_st | −0.346953682 | 4:8007O28-8007108 (−) | 22 | UUCAACGGGUAUUUAUUGAGCA | 128 |
| hsa-miR-96_st | 1.039744333 | 7:129414532-129414609 (−) | 23 | UUUGGCACUAGCACAUUUUUGCU | 129 |
| hsa-miR-96- | −0.156917058 | 7:129414532-129414609 (−) | 22 | AAUCAUGUGCAGUGCCAAUAUG | 130 |
| star_st | | | | | |
| hsa-miR-98_st | 0.922592035 | X:53583184-53583302 (−) | 22 | UGAGGUAGUAAGUUGUAUUGUU | 131 |
| hsa-miR-99a_st | 0.512778282 | 21:17911409-17911489 (+) | 22 | AACCCGUAGAUCCGAUCUUGUG | 132 |
| hsa-miR-99a- | 0.881793689 | 21:17911409-17911489 (+) | 22 | CAAGCUCGCUUCUAUGGGUCUG | 133 |
| star_st | | | | | |
| hsa-miR-100_st | 0.249572435 | 11:122022937-122023016 (−) | 22 | AACCCGUAGAUCCGAACUUGUG | 134 |
| hsa-miR-100- | −0.271273201 | 11:122022937-122023016 (−) | 22 | CAAGCUUGUAUCUAUAGGUAUG | 135 |
| star_st | | | | | |
| hsa-miR-101- | 0.365359575 | | 22 | CAGUUAUCACAGUGCUGAUGCU | 136 |
| star_st | | | | | |
| hsa-miR-101_st | 0.821576153 | | 21 | UACAGUACUGUGAUAACUGAA | 137 |
| hsa-miR-29b-1- | 0.465671107 | | 24 | GCUGGUUUCAUAUGGUGGUUUAGA | 138 |
| star_st | | | | | |
| hsa-miR-29b_st | 0.950973534 | | 23 | UAGCACCAUUUGAAAUCAGUGUU | 139 |
| hsa-miR-29b-2- | 0.072729757 | | 22 | CUGGUUUCACAUGGUGGCUUAG | 140 |
| star_st | | | | | |
| hsa-miR-103-2- | 0.47954039 | | 23 | AGCUUCUUUACAGUGCUGCCUUG | 141 |
| star_st | | | | | |
| hsa-miR-103_st | 0.492177895 | | 23 | AGCAGCAUUGUACAGGGCUAUGA | 142 |
| hsa-miR-105_st | 0.012220407 | | 23 | UCAAAUGCUCAGACUCCUGUGGU | 143 |
| hsa-miR-105- | −0.171838427 | | 22 | ACGGAUGUUUGAGCAUGUGCUA | 144 |
| star_st | | | | | |
| hsa-miR-106a_st | 0.641800789 | X:133304228-133304308 (−) | 23 | AAAAGUGCUUACAGUGCAGGUAG | 145 |
| hsa-miR-106a- | −0.002080901 | X:133309228-133304308 (−) | 22 | CUGCAAUGUAAGCACUUCUUAC | 146 |
| star_st | | | | | |
| hsa-miR-107_st | 0.57762915 | 10:91352504-91352584 (−) | 23 | AGCAGCAUUGUACAGGGCUAUCA | 147 |
| hsa-miR-16-2- | 0.890157867 | | 22 | CCAAUAUUACUGUGCUGCUUUA | 148 |
| star_st | | | | | |
| hsa-miR-192_st | 0.593710572 | 11:64658609-64658718 (−) | 21 | CUGACCUAUGAAUUGACAGCC | 149 |
| hsa-miR-192- | 0.219711485 | 11:64658609-64658718 (−) | 22 | CUGCCAAUUCCAUAGGUCACAG | 150 |
| star_st | | | | | |
| hsa-miR-196a_st | 0.997020636 | | 22 | UAGGUAGUUUCAUGUUGUUGGG | 151 |
| hsa-miR-197_st | −0.695426484 | 1:110141515-110141589 (+) | 22 | UUCACCACCUUCUCCACCCAGC | 152 |
| hsa-miR-198_st | −0.374203131 | 3:120114515-120114576 (−) | 22 | GGUCCAGAGGGGAGAUAGGUUC | 153 |
| hsa-miR-199a- | 0.924255382 | | 23 | CCCAGUGUUCAGACUACCUGUUC | 154 |
| 5p_st | | | | | |
| hsa-miR-199a- | 0.815769594 | | 22 | ACAGUAGUCUGCACAUUGGUUA | 155 |
| 3p_st | | | | | |
| hsa-miR-208a_st | 0.43995374 | 14:23857805-23857875 (−) | 22 | AUAAGACGAGCAAAAAGCUUGU | 156 |
| hsa-miR-129- | −0.640994824 | | 21 | CUUUUUGCGGUCUGGGCUUGC | 157 |
| 5p_st | | | | | |
| hsa-miR-129- | 0.229379183 | | 22 | AAGCCCUUACCCCAAAAAGUAU | 158 |
| star_st | | | | | |
| hsa-miR-148a- | 0.244582293 | 7:25989539-25989606 (−) | 22 | AAAGUUCUGAGACACUCCGACU | 159 |
| star_st | | | | | |
| hsa-miR-148a_st | 0.823898116 | 7:25989539-25989606 (−) | 22 | UCAGUGCACUACAGAACUUUGU | 160 |
| hsa-miR-30c_st | −0.222005041 | | 23 | UGUAAACAUCCUACACUCUCAGC | 161 |
| hsa-miR-30c-2- | −0.403215672 | | 22 | CUGGGAGAAGGCUGUUUACUCU | 162 |
| star_st | | | | | |
| hsa-miR-30d_st | 0.367431688 | 8:135817119-135817188 (−) | 22 | UGUAAACAUCCCCGACUGGAAG | 163 |
| hsa-miR-30d- | 0.274085418 | 8:135817119-135817188 (−) | 22 | CUUUCAGUCAGAUGUUUGCUGC | 164 |
| star_st | | | | | |
| hsa-miR-139- | −1.091859388 | 11:72326107-72326174 (−) | 22 | UCUACAGUGCACGUGUCUCCAG | 165 |
| 5p_st | | | | | |
| hsa-miR-139- | −0.547607554 | 11:72326107-72326174 (−) | 22 | GGAGACGCGGCCCUGUUGGAGU | 166 |
| 3p_st | | | | | |
| hsa-miR-147_st | −0.258983494 | 9:123007257-123007328 (−) | 20 | GUGUGUGGAAAUGCUUCUGC | 167 |
| hsa-miR-7_st | −2.160993031 | | 23 | UGGAAGACUAGUGAUUUUGUUGU | 168 |
| hsa-miR-7-1- | 0.390819845 | | 22 | CAACAAAUCACAGUCUGCCAUA | 169 |
| star_st | | | | | |
| hsa-miR-7-2- | −0.891725706 | | 22 | CAACAAAUCCCAGUCUACCUAA | 170 |
| star_st | | | | | |
| hsa-miR-10a_st | 0.760462994 | 17:46657200-46657309 (−) | 23 | UACCCUGUAGAUCCGAAUUUGUG | 171 |
| hsa-miR-10a- | 0.187536432 | 17:46657200-46657309 (−) | 22 | CAAAUUCGUAUCUAGGGGAAUA | 172 |
| star_st | | | | | |
| hsa-miR-10b_st | 1.192856815 | 2:177015031-177015140 (+) | 23 | UACCCUGUAGAACCGAAUUUGUG | 173 |
| hsa-miR-10b- | 0.27457053 | 2:177015031-177015140 (+) | 22 | ACAGAUUCGAUUCUAGGGGAAU | 174 |
| star_st | | | | | |
| hsa-miR-34a_st | 1.644540443 | 1:9211727-9211836 (−) | 22 | UGGCAGUGUCUUAGCUGGUUGU | 175 |
| hsa-miR-34a- | 1.510148362 | 1:9211727-9211836 (−) | 22 | CAAUCAGCAAGUAUACUGCCCU | 176 |
| star_st | | | | | |
| hsa-miR-181a_st | 0.493363018 | | 23 | AACAUUCAACGCUGUCGGUGAGU | 177 |
| hsa-miR-181a-2- | 1.02626003 | | 22 | ACCACUGACCGUUGACUGUACC | 178 |
| star_st | | | | | |
| hsa-miR-181b_st | 0.527375559 | | 23 | AACAUUCAUUGCUGUCGGUGGGU | 179 |
| hsa-miR-181c_st | 1.011868821 | 19:13985513-13985622 (+) | 22 | AACAUUCAACCUGUCGGUGAGU | 180 |
| hsa-miR-181c- | 0.59869708 | 19:13985513-13985622 (+) | 22 | AAACCAUCGACCGUUGAGUGGAC | 181 |
| star_st | | | | | |
| hsa-miR-182_st | 1.262246153 | 7:129410223-129410332 (−) | 24 | UUUGGCAAUGGUAGAACUCACACU | 182 |
| hsa-miR-182- | −0.343312162 | 7:129410223-129410332 (−) | 21 | UGGUUCUAGACUUGCCAACUA | 183 |
| star_st | | | | | |
| hsa-miR-183_st | 0.630655391 | 7:129414745-129414854 (−) | 22 | UAUGGCACUGGUAGAAUUCACU | 184 |
| hsa-miR-183- | −0.13345253 | 7:129414745-129414854 (−) | 22 | GUGAAUUACCGAAGGGCCAUAA | 185 |
| star_st | | | | | |
| hsa-miR-187- | −0.490744534 | 18:33484781-33484889 (−) | 22 | GGCUACAACACAGGACCCGGGC | 186 |
| star_st | | | | | |
| hsa-miR-187_st | 0.28384338 | 18:33484781-33484889 (−) | 22 | UCGUGUCUUGUGUUGCAGCCGG | 187 |
| hsa-miR-196a- | 0.574631487 | | 22 | CGGCAACAAGAAACUGCCUGAG | 188 |
| star_st | | | | | |
| hsa-miR-199b- | 0.931725352 | 9:131007000-131007109 (−) | 23 | CCCAGUGUUUAGACUAUCUGUUC | 189 |
| 5p_st | | | | | |
| hsa-miR-199b- | 1.008638211 | 9:131007000-131007109 (−) | 22 | ACAGUAGUCUGCACAUUGGUUA | 190 |
| 3p_st | | | | | |
| hsa-miR-203_st | 0.035466475 | 14:104583742-104583851 (+) | 22 | GUGAAAUGUUUAGGACCACUAG | 191 |
| hsa-miR-204_st | −1.642891797 | 9:73424891-73425000 (−) | 22 | UUCCCUUUGUCAUCCUAUGCCU | 192 |
| hsa-miR-205_st | 0.365322787 | 1:209605478-209605587 (+) | 22 | UCCUUCAUUCCACCGGAGUCUG | 193 |
| hsa-miR-205- | −0.100505489 | 1:209605478-209605587 (+) | 21 | GAUUUCAGUGGAGUGAAGUUC | 194 |
| star_st | | | | | |
| hsa-miR-210_st | −0.426055268 | 11:568089-568198 (−) | 22 | CUGUGCGUGUGACAGCGGCUGA | 195 |
| hsa-miR-211_st | −0.207934914 | 15:31357235-31357344 (−) | 22 | UUCCCUUUGUCAUCCUUCGCCU | 196 |
| hsa-miR-212_st | 0.387277691 | 17:1953565-1953674 (−) | 21 | UAACAGUCUCCAGUCACGGCC | 197 |
| hsa-miR-181a- | 1.217432668 | | 22 | ACCAUCGACCGUUGAUUGUACC | 198 |
| star_st | | | | | |
| hsa-miR-214- | 0.664208274 | 1:172107938-172108047 (−) | 22 | UGCCUGUCUACACUUGCUGUGC | 199 |
| star_st | | | | | |
| hsa-miR-214_st | −0.192700009 | 1:172107938-172108047 (−) | 22 | ACAGCAGGCACAGACAGGCAGU | 200 |
| hsa-miR-215_st | 0.760307473 | 1:220291195-220291304 (−) | 21 | AUGACCUAUGAAUUGACAGAC | 201 |
| hsa-miR7216a_st | 0.208935083 | 2:56216085-56216194 (−) | 22 | UAAUCUCAGCUGGCAACUGUGA | 202 |
| hsa-miR-217_st | 0.295493985 | 2:56210102-56210211 (−) | 23 | UACUGCAUCAGGAACUGAUUGGA | 203 |
| hsa-miR-218_st | 1.215558827 | | 21 | UUGUGCUUGAUCUAACCAUGU | 204 |
| hsa-miR-218-1- | −0.402759664 | | 22 | AUGGUUCCGUCAAGCACCAUGG | 205 |
| star_st | | | | | |
| hsa-miR-218-2- | −0.349252359 | | 22 | CAUGGUUCUGUCAAGCACCGCG | 206 |
| star_st | | | | | |
| hsa-miR-219- | 0.023609757 | | 21 | UGAUUGUCCAAACGCAAUUCU | 207 |
| 5p_st | | | | | |
| hsa-miR-219-1- | 0.252378496 | 6:33175612-33175721 (+) | 22 | AGAGUUGAGUCUGGACGUCCCG | 208 |
| 3p_st | | | | | |
| hsa-miR-220a_st | 0.112818758 | X:122695946-122696055 (−) | 21 | CCACACCGUAUCUGACACUUU | 209 |
| hsa-miR-221- | 2.715957711 | X:45605585-45605694 (−) | 22 | ACCUGGCAUACAAUGUAGAUUU | 210 |
| star_st | | | | | |
| hsa-miR-221_st | 1.897354614 | X:45605585-45605694 (−) | 23 | AGCUACAUUGUCUGCUGGGUUUC | 211 |
| hsa-miR-222- | 0.805099621 | X:45606421-45606530 (−) | 22 | CUCAGUAGCCAGUGUAGAUCCU | 212 |
| star_st | | | | | |
| hsa-miR-222_st | 1.96393452 | X:45606421-45606530 (−) | 21 | AGCUACAUCUGGCUACUGGGU | 213 |
| hsa-miR-223- | 0.532413464 | X:65238712-65238821 (+) | 22 | CGUGUAUUUGACAAGCUGAGUU | 214 |
| star_st | | | | | |
| hsa-miR-223_st | 0.624421002 | X:65238712-65238821 (+) | 22 | UGUCAGUUUGUCAAAUACCCCA | 215 |
| hsa-miR-224_st | 0.990921161 | X:151127050-151127130 (−) | 21 | CAAGUCACUAGUGGUUCCGUU | 216 |
| hsa-miR-224- | 0.31235436 | X:151127050-151127130 (−) | 23 | AAAAUGGUGCCCUAGUGACUACA | 217 |
| star_st | | | | | |
| hsa-miR-200b- | −0.404959657 | 1:1102484-1102578 (+) | 22 | CAUCUUACUGGGCAGCAUUGGA | 218 |
| star_st | | | | | |
| hsa-miR-200b_st | 0.17647889 | 1:1102484-1102578 (+) | 22 | UAAUACUGCCUGGUAAUGAUGA | 219 |
| hsa-let-7g_st | 0.180746211 | 3:52302294-52302377 (−) | 22 | UGAGGUAGUAGUUUGUACAGUU | 220 |
| hsa-let-7g- | −0.360572088 | 3:52302294-52302377 (−) | 21 | CUGUACAGGCCACUGCCUUGC | 221 |
| star_st | | | | | |
| hsa-10-7i_st | 0.416034157 | 12:62997466-62997549 (+) | 22 | UGAGGUAGUAGUUUGUGCUGUU | 222 |
| hsa-14-7i- | 0.388065899 | 12:62997466-62997549 (+) | 22 | CUGCGCAAGCUACUGCCUUGCU | 223 |
| star_st | | | | | |
| hsa-miR-1_st | 0.061353305 | | 22 | UGGAAUGUAAAGAAGUAUGUAU | 224 |
| hsa-miR-15b_st | −0.003846858 | 3:160122376-160122473 (+) | 22 | UAGCAGCACAUCAUGGUUUACA | 225 |
| hsa-miR-15b- | −0.526285658 | 3:160122376-160122473 (+) | 22 | CGAAUCAUUAUUUGCUGCUCUA | 226 |
| star_st | | | | | |
| hsa-miR-23b- | −0.045511684 | 9:97847490-97847586 (+) | 22 | UGGGUUCCUGGCAUGCUGAUUU | 227 |
| star_st | | | | | |
| hsa-miR-23b_st | 0.453860873 | 9:97847490-97847586 (+) | 21 | AUCACAUUGCCAGGGAUUACC | 228 |
| hsa-miR-27b- | 0.988333417 | 9:97847727-97847823 (+) | 22 | AGAGCUUAGCUGAUUGGUGAAC | 229 |
| star_st | | | | | |
| hsa-miR-27b_st | 0.944920392 | 9:97847727-97847823 (+) | 21 | UUCACAGUGGCUAAGUUCUGC | 230 |
| hsa-miR-30b_st | 0.400951157 | 8:135812763-135812850 (−) | 22 | UGUAAACAUCCUACACUCAGCU | 231 |
| hsa-miR-30b- | −0.446186417 | 8:135812763-135812850 (−) | 22 | CUGGGAGGUGGAUGUUUACUUC | 232 |
| star_st | | | | | |
| hsa-miR-122_st | −0.528270521 | 18:56118306-56118390 (+) | 22 | UGGAGUGUGACAAUGGUGUUUG | 233 |
| hsa-miR-122- | 0.218095686 | 18:56118306-56118390 (+) | 22 | AACGCCAUUAUCACACUAAAUA | 234 |
| star_st | | | | | |
| hsa-miR-124- | −0.098340343 | | 22 | CGUGUUCACAGCGGACCUUGAU | 235 |
| star_st | | | | | |
| hsa-miR-124_st | −0.171384948 | | 20 | UAAGGCACGCGGUGAAUGCC | 236 |
| hsa-miR-125b_st | 0.07339278 | | 22 | UCCCUGAGACCCUAACUUGUGA | 237 |
| hsa-miR-125b-1- | −0.44737633 | | 22 | ACGGGUUAGGCUCUUGGGAGCU | 238 |
| star_st | | | | | |
| hsa-miR-128_st | 0.375130151 | | 21 | UCACAGUGAACCGGUCUCUUU | 239 |
| hsa-miR-130a- | −0.075902575 | 11:57408671-57408759 (+) | 22 | UUCACAUUGUGCUACUGUCUGC | 240 |
| star_st | | | | | |
| hsa-miR-130a_st | 0.118023189 | 11:57408671-57408159 (+) | 22 | CAGUGCAAUGUUAAAAGGGCAU | 241 |
| hsa-miR-132- | 0.996501258 | 17:1953202-1953302 (−) | 22 | ACCGUGGCUUUCGAUUGUUACU | 242 |
| star_st | | | | | |
| hsa-miR-132_st | 1.065793699 | 17:1953202-1953302 (−) | 22 | UAACAGUCUACAGCCAUGGUCG | 243 |
| hsa-miR-133a_st | −0.425346456 | | 22 | UUUGGUCCCCUUCAACCAGCUG | 244 |
| hsa-miR-135a_st | 1.06098116 | | 23 | UAUGGCUUUUUAUUCCUAUGUGA | 245 |
| hsa-miR-135a- | −0.505118465 | | 22 | UAUAGGGAUUGGAGCCGUGGCG | 246 |
| star_st | | | | | |
| hsa-miR-137_st | 0.237080977 | 1:98511626-98511727 (−) | 23 | UUAUUGCUUAAGAAUACGCGUAG | 247 |
| hsa-miR-138_st | −0.960265771 | | 23 | AGCUGGUGUUGUGAAUCAGGCCG | 248 |
| hsa-miR-138-2- | 0.256034416 | | 22 | GCUAUUUCACGACACCAGGGUU | 249 |
| star_st | | | | | |
| hsa-miR-140- | 1.130300163 | 16:69966984-69967083 (+) | 22 | CAGUGGUUUUACCCUAUGGUAG | 250 |
| 5p_st | | | | | |
| hsa-miR-140- | 0.223036719 | 16:69966984-69967083 (+) | 21 | UACCACAGGGUAGAACCACGG | 251 |
| 3p_st | | | | | |
| hsa-miR-141- | −0.008343285 | 12:7073260-7073354 (+) | 22 | CAUCUUCCAGUACAGUGUUGGA | 252 |
| star_st | | | | | |
| hsa-miR-141_st | 0.450479229 | 12:7073260-7073354 (+) | 22 | UAACACUGUCUGGUAAAGAUGG | 253 |
| hsa-miR-142- | 1.353572052 | 17:56408593-56408679 (−) | 21 | CAUAAAGUAGAAAGCACUACU | 254 |
| 5p_st | | | | | |
| hsa-miR-142- | 0.246580674 | 17:56408593-56408679 (−) | 23 | UGUAGUGUUUCCUACUUUAUGGA | 255 |
| 3p_st | | | | | |
| hsa-miR-143- | −0.064880327 | 5:148808481-148808586 (+) | 22 | GGUGCAGUGCUGCAUCUCUGGU | 256 |
| star_st | | | | | |
| hsa-miR-143_st | 0.245792956 | 5:148808481-148808386 (+) | 21 | UGAGAUGAAGCACUGUAGCUC | 257 |
| hsa-miR-144- | 0.02638293 | 17:27188551-27188636 (−) | 22 | GGAUAUCAUCAUAUACUGUAAG | 258 |
| star_st | | | | | |
| hsa-miR-144_st | −0.474283509 | 17:27188551-27188636 (−) | 20 | UACAGUAUAGAUGAUGUACU | 259 |
| hsa-miR-145_st | −0.331497872 | 5:148810209-148810296 (+) | 23 | GUCCAGUUUUCCCAGGAAUCCCU | 260 |
| hsa-miR-145- | 0.289009546 | 5:148810209-148810296 (+) | 22 | GGAUUCCUGGAAAUACUGUUCU | 261 |
| star_st | | | | | |
| hsa-miR-152_st | −0.132107943 | 17:46114527-46114613 (−) | 21 | UCAGUGCAUGACAGAACUUGG | 262 |
| hsa-miR-153_st | 0.206204323 | | 22 | UUGCAUAGUCACAAAAGUGAUC | 263 |
| hsa-miR-191_st | 0.527445159 | 3:49058051-49038142 (−) | 23 | CAACGGAAUCCCAAAAGCAGCUG | 264 |
| hsa-miR-191- | −0.177276096 | 3:49058051-49058142 (−) | 22 | GCUGCGCUUGGAUUUCGUCCC | 265 |
| star_st | | | | | |
| hsa-miR-9_st | 0.361062989 | | 23 | UCUUUGGUUAUCUAGCUGUAUGA | 266 |
| hsa-miR-9- | 0.129542783 | | 22 | AUAAAGCUAGAUAACCGAAAGU | 267 |
| star_st | | | | | |
| hsa-miR-125a- | 0.027803044 | 19:52196507-52196592 (+) | 24 | UCCCUGAGACCCUUUAACCUGUGA | 268 |
| 5p_st | | | | | |
| hsa-miR-125a- | −0.356803841 | 19:52196507-52196592 (+) | 22 | ACAGGUGAGGUUCUUGGGAGCC | 269 |
| 3p_st | | | | | |
| hsa-miR-125b- | −0.201602688 | | 22 | UCACAAGUCAGGCUCUUGGGAC | 270 |
| 2-star_st | | | | | |
| hsa-miR-126- | 0.576116104 | 9:139565054-139363138 (+) | 21 | CAUUAUUACUUUUGGUACGCG | 271 |
| star_st | | | | | |
| hsa-miR-126_st | −0.007129146 | 9:139565054-139565138 (+) | 22 | UCGUACCGUGAGUAAUAAUGCG | 272 |
| hsa-miR-127- | 0.43991996 | 14:101349316-101349412 (+) | 22 | CUGAAGCUCAGAGGGCUCUGAU | 273 |
| 5p_st | | | | | |
| hsa-miR-127- | 0.539861029 | 14:101349316-101349412 (+) | 22 | UCGGAUCCGUCUGAGCUUGGCU | 274 |
| 3p_st | | | | | |
| hsa-miR-129- | 0.651300542 | | 22 | AAGCCCUUACCCCAAAAAGGAU | 275 |
| 3p_st | | | | | |
| hsa-miR-134_st | −0.263214268 | 14:101521024-101521096 (+) | 22 | UGUGACUGGUUGACCAGAGGGG | 276 |
| hsa-miR-136_st | 0.314078184 | 14:101351039-101351120 (+) | 23 | ACUCCAUUUGUUUUGAUGAUGGA | 277 |
| hsa-miR-136- | 0.458516901 | 14:101351039-101351120 (+) | 22 | CAUCAUCGUCUCAAAUGAGUCU | 278 |
| star_st | | | | | |
| hsa-miR-138-1- | −0.910488475 | | 22 | GCUACUUCACAACACCAGGGCC | 279 |
| star_st | | | | | |
| hsa-miR-146a_st | 1.489357674 | 5:159912359-159912457 (+) | 22 | UGAGAACUGAAUUCCAUGGGUU | 280 |
| hsa-miR-146a- | 0.839196309 | 5:159912359-159912457 (+) | 22 | CCUCUGAAAUUCAGUUCUUCAG | 281 |
| star_st | | | | | |
| hsa-miR-149_st | 0.262228024 | 2:241395418-241395506 (+) | 23 | UCUGGCUCCGUGUCUUCACUCCC | 282 |
| hsa-miR-149- | −0.494806596 | 2:241395418-241195506 (+) | 21 | AGGGAGGGACGGGGGCUGUGC | 283 |
| star_st | | | | | |
| hsa-miR-150_st | −0.136159708 | 19:50004042-50004125 (−) | 22 | UCUCCCAACCCUUGUACCAGUG | 284 |
| hsa-miR-150- | −0.349080262 | 19:50004042-50004125 (−) | 22 | CUGGUACAGGCCUGGGGGACAG | 285 |
| star_st | | | | | |
| hsa-miR-154_st | −0.037793351 | 14:101526092-101526175 (+) | 22 | UAGGUUAUCCGUGUUGCCUUCG | 286 |
| hsa-miR-154- | −0.212570871 | 14:101526092-101526175 (+) | 22 | AAUCAUACACGGUUGACCUAUU | 287 |
| star_st | | | | | |
| hsa-miR-184_st | −0.408453827 | 15:19502130-79502213 (+) | 22 | UGGACGGAGAACUGAUAAGGGU | 288 |
| hsa-miR-185_st | −0.11768006 | 22:20020662-20020743 (+) | 22 | UGGAGAGAAAGGCAGUUCCUGA | 289 |
| hsa-miR-185- | −0.973967372 | 22:20020662-20020743 (+) | 22 | AGGGGCUGGCUUUCCUCUGGUC | 290 |
| star_st | | | | | |
| hsa-miR-186_st | −0.167692325 | 1:71533314-71533399 (−) | 22 | CAAAGAAUUCUCCUUUUGGGCU | 291 |
| hsa-miR-186- | 0.75235132 | 1:71533314-71533399 (−) | 22 | GCCCAAAGGUGAAUUUUUUGGG | 292 |
| star_st | | | | | |
| hsa-miR-188- | 0.153429662 | X:49768109-49768194 (+) | 21 | CAUCCCUUGCAUGGUGGAGGG | 293 |
| 5p_st | | | | | |
| hsa-miR-188- | 0.134349045 | X:49768109-49768194 (+) | 21 | CUCCACAUGCAGGGUUUGCA | 294 |
| 3p_st | | | | | |
| hsa-miR-190_st | −0.24523743 | 15:63116156-63116240 (+) | 22 | UGAUAUGUUUGAUAUAUUAGGU | 295 |
| hsa-miR-193a- | −0.833145932 | 17:29887015-29887102 (+) | 22 | UGGGUCUUUGCGGGCGAGAUGA | 296 |
| 5p_st | | | | | |
| hsa-miR-193a- | 0.238877874 | 17:29887015-29887102 (+) | 22 | AACUGGCCUACAAAGUCCCAGU | 297 |
| 3p_st | | | | | |
| hsa-miR-194_st | 0.379322206 | | 22 | UGUAACAGCAACUCCAUGUGGA | 298 |
| hsa-miR-195_st | 0.344977836 | 17:6920934-6921020 (−) | 21 | UAGCAGCACAGAAAUAUUGGC | 299 |
| hsa-miR-195- | 0.40065597 | 17:6920934-6921020 (−) | 22 | CCAAUAUUGGCUGUGCUGCUCC | 300 |
| star_st | | | | | |
| hsa-miR-206_st | 0.08440626 | 6:52009147-52009232 (+) | 22 | UGGAAUGUAAGGAAGUGUGUGG | 301 |
| hsa-miR-320a_st | −0.306150576 | 8:22102475-22102556 (−) | 22 | AAAAGCUGGGUUGAGAGGGCGA | 302 |
| hsa-miR-200c- | −0.052932563 | 12:7072862-1072929 (+) | 22 | CGUCUUACCCAGCAGUGUUUGG | 303 |
| star_st | | | | | |
| hsa-miR-200c_st | −0.239821076 | 12:7072862-7072929 (+) | 23 | UAAUACUGCCGGGUAAUGAUGGA | 304 |
| hsa-miR-155_st | 0.608153889 | 21:26946292-26946356 (+) | 23 | UUAAUGCUAAUCGUGAUAGGGGU | 305 |
| hsa-miR-155- | 0.056816503 | 21:26946292-26946356 (+) | 22 | CUCCUACAUAUUAGCAUUAACA | 306 |
| star_st | | | | | |
| hsa-miR-194- | −0.515277592 | | 22 | CCAGUGGGGCUGCUGUUAUCUG | 307 |
| star_st | | | | | |
| hsa-miR-106b_st | 0.896125094 | 7:99691616-99691697 (−) | 21 | UAAAGUGCUGACAGUGCAGAU | 308 |
| hsa-miR-106b- | 0.137425123 | 7:99691616-99691697 (−) | 22 | CCGCACUGUGGGUACUUGCUGC | 309 |
| star_st | | | | | |
| hsa-miR-29c- | 0.140772995 | 1:207975197-207975284 (−) | 22 | UGACCGAUUUCUCCUGGUGUUC | 310 |
| star_st | | | | | |
| hsa-miR-29c_st | 0.436946695 | 1:207975197-207975284 (−) | 22 | UAGCACCAUUUGAAAUCGGUUA | 311 |
| hsa-miR-30c-1- | −0.049900136 | | 22 | CUGGGAGAGGGUUGUUUACUCC | 312 |
| star_st | | | | | |
| hsa-miR-200a- | 0.257167734 | 1:1103243-1103332 (+) | 22 | CAUCUUACCGGACAGUGCUGGA | 313 |
| star_st | | | | | |
| hsa-miR-200a_st | 0.334093033 | 1:1103243-1103332 (+) | 22 | UAACACUGUCUGGUAACGAUGU | 314 |
| hsa-miR-302a- | −0.001562424 | 4:113569339-113569407 (−) | 23 | ACUUAAACGUGGAUGUACUUGCU | 315 |
| star_st | | | | | |
| hsa-miR-302a_st | 0.182673435 | 4:113569339-113569407 (−) | 23 | UAAGUGCUUCCAUGUUUUGGUGA | 316 |
| hsa-miR-219-2- | 0.008465241 | 9:131154897-131154993 (−) | 22 | AGAAUUGUGGCUGGACAUCUGU | 317 |
| 3p_st | | | | | |
| hsa-miR-34b- | 1.378749354 | 11:111383663-111383746 (+) | 23 | UAGGCAGUGUCAUUAGCUGAUUG | 318 |
| star_st | | | | | |
| hsa-miR-34b_st | −0.604360179 | 11:111383663-111383746 (+) | 22 | CAAUCACUAACUCCACUGCCAU | 319 |
| hsa-miR-34c- | 0.588298823 | 11:111384164-111384240 (+) | 23 | AGGCAGUGUAGUUAGCUGAUUGC | 320 |
| 5p_st | | | | | |
| hsa-miR-34c- | −0.181566213 | 11:111384164-111384240 (+) | 22 | AAUCACUAACCACACGGCCAGG | 321 |
| 3p_st | | | | | |
| hsa-miR-299- | −0.15952566 | 14:101490131-101490193 (+) | 22 | UGGUUUACCGUCCCACAUACAU | 322 |
| 5p_st | | | | | |
| hsa-miR-299- | −0.134298858 | 14:101490131-101490193 (+) | 22 | UAUGUGGGAUGGUAAACCGCUU | 323 |
| 3p_st | | | | | |
| hsa-miR-301a_st | 1.480810077 | 17:57228497-57228582 (−) | 23 | CAGUGCAAUAGUAUUGUCAAAGC | 324 |
| hsa-miR-99b_st | 0.445102969 | 19:52195865-52195934 (+) | 22 | CACCCGUAGAACCGACCUUGCG | 325 |
| hsa-miR-99b- | −0.256000929 | 19:52195865-52195934 (+) | 22 | CAAGCUCGUGUCUGUGGGUCCG | 326 |
| star_st | | | | | |
| hsa-miR-296- | 0.331136889 | 20:57392670-57392749 (−) | 21 | AGGGCCCCCCCUCAAUCCUGU | 327 |
| 5p_st | | | | | |
| hsa-miR-296- | −0.484459617 | 20:57392670-57392749 (−) | 22 | GAGGGUUGGGUGGAGGCUCUCC | 328 |
| 3p_st | | | | | |
| hsa-miR-130b- | −0.428478249 | 22:22007593-22007674 (+) | 21 | ACUCUUUCCCUGUUGCACUAC | 329 |
| star_st | | | | | |
| hsa-miR-130b_st | 0.276541886 | 22:22007593-22007674 (+) | 22 | CAGUGCAAUGAUGAAAGGGCAU | 330 |
| hsa-miR-30c_st | 0.367656808 | 1:41220027-41220118 (+) | 22 | UGUAAACAUCCUUGACUGGAAG | 331 |
| hsa-miR-30c- | −0.524125402 | 1:41220027-41220118 (+) | 22 | CUUUCAGUCGGAUGUUUACAGC | 332 |
| star_st | | | | | |
| hsa-miR-26a-2- | 0.43879887 | | 22 | CCUAUUCUUGAUUACUUGUUUC | 333 |
| star_st | | | | | |
| hsa-miR-361- | 0.185895719 | X:85158641-85158712 (−) | 22 | UUAUCAGAAUCUCCAGGGGUAC | 334 |
| 5p_st | | | | | |
| hsa-miR-361- | 0.280893435 | X:85158641-85158712 (−) | 23 | UCCCCCAGGUGUGAUUCUGAUUU | 335 |
| 3p_st | | | | | |
| hsa-miR-362- | 0.289322897 | X:49773572-49773636 (+) | 24 | AAUCCUUGGAACCUAGGUGUGAGU | 336 |
| 5p_st | | | | | |
| hsa-miR-362- | 0.932533015 | X:49773572-49773636 (+) | 22 | AACACACCUAUUCAAGGAUUCA | 337 |
| 3p_st | | | | | |
| hsa-miR-363- | −0.421094689 | X:133303408-133303482 (−) | 22 | CGGGUGGAUCACGAUGCAAUUU | 338 |
| star_st | | | | | |
| hsa-miR-363_st | 0.57250165 | X:133303408-133303482 (−) | 22 | AAUUGCACGGUAUCCAUCUGUA | 339 |
| hsa-miR-365_st | −0.083976508 | | 22 | UAAUGCCCCUAAAAAUCCUUAU | 340 |
| hsa-miR-365- | −0.543569131 | | 22 | AGGGACUUUCAGGGGCAGCUGU | 341 |
| star_st | | | | | |
| hsa-miR-302b- | −0.079212528 | 4:113569641-113569713 (−) | 22 | ACUUUAACAUGGAAGUGCUUUC | 342 |
| star_st | | | | | |
| hsa-miR-302b_st | 0.802790198 | 4:113569641-113569713 (−) | 23 | UAAGUGCUUCCAUGUUUUAGUAG | 343 |
| hsa-miR-302c- | −0.544478956 | 4:113569519-113569586 (−) | 22 | UUUAACAUGGGGGUACCUGCUG | 344 |
| star_st | | | | | |
| hsa-miR-302c_st | 0.501592778 | 4:113569519-113569586 (−) | 23 | UAAGUGCUUCCAUGUUUCAGUGG | 345 |
| hsa-miR-302d- | −0.103409044 | 4:113569160-113569227 (−) | 22 | ACUUUAACAUGGAGGCACUUGC | 346 |
| star_st | | | | | |
| hsa-miR-302d_st | 0.48017505 | 4:113569160-113569227 (−) | 23 | UAAGUGCUUCCAUGUUUGAGUGU | 347 |
| hsa-miR-367- | −0.000436563 | 4:113569030-113569097 (−) | 22 | ACUGUUGCUAAUAUGCAACUCU | 348 |
| star_st | | | | | |
| hsa-miR-367_st | −0.023708527 | 4:113569030-113569097 (−) | 22 | AAUUGCACUUUAGCAAUGGUGA | 349 |
| hsa-miR-376c_st | 1.223654497 | 14:101506027-101506092 (+) | 21 | AACAUAGAGGAAAUUCCACGU | 350 |
| hsa-miR-369- | 0.853092711 | 14:101531935-101532004 (+) | 22 | AGAUCGACCGUGUUAUAUUCGC | 351 |
| 5p_st | | | | | |
| hsa-miR-369- | −0.381025345 | 14:101531935-101532004 (+) | 21 | AAUAAUACAUGGUUGAUCUUU | 352 |
| 3p_st | | | | | |
| hsa-miR-370_st | −0.485633939 | 14:101377476-101377550 (+) | 22 | GCCUGCUGGGGUGGAACCUGGU | 353 |
| hsa-miR-371- | −0.699590356 | 19:54290929-54290995 (+) | 20 | ACUCAAACUGUGGGGGCACU | 354 |
| 5p_st | | | | | |
| hsa-miR-371- | −0.767486246 | 19:54290929-54290995 (+) | 23 | AAGUGCCGCCAUCUUUUGAGUGU | 355 |
| 3p_st | | | | | |
| hsa-miR-372_st | −0.018614281 | 19:54291144-54291210 (+) | 23 | AAAGUGCUGCGACAUUUGAGCGU | 356 |
| hsa-miR-373- | −0.91382708 | 19:54291959-54292027 (+) | 22 | ACUCAAAAUGGGGGCGCUUUCC | 357 |
| star_st | | | | | |
| hsa-miR-373_st | 0.061600195 | 19:54291959-54292027 (+) | 23 | GAAGUGCUUCGAUUUUGGGGUGU | 358 |
| hsa-miR-374a_st | 0.500957184 | X:73507121-73507192 (−) | 22 | UUAUAAUACAACCUGAUAAGUG | 359 |
| hsa-miR-374- | −0.007451471 | X:73507121-73507192 (−) | 22 | CUUAUCAGAUUGUAUUGUAAUU | 360 |
| star_st | | | | | |
| hsa-miR-375_st | 2.428793377 | 2:219866367-219866430 (−) | 22 | UUUGUUCGUUCGGCUCGCGUGA | 361 |
| hsa-miR-376a- | −0.114680818 | | 22 | GUAGAUUCUCCUUCUAUGAGUA | 362 |
| star_st | | | | | |
| hsa-miR-376a_st | 0.657126002 | | 21 | AUCAUAGAGGAAAAUCCACGU | 363 |
| hsa-miR-377- | 0.133361438 | 14:101528387-101528455 (+) | 22 | AGAGGUUGCCCUUGGUGAAUUC | 364 |
| star_st | | | | | |
| hsa-miR-377_st | 0.547689033 | 14:101528387-101528455 (+) | 22 | AUCACACAAAGGCAACUUUUGU | 365 |
| hsa-miR-378- | −0.137066823 | 5:149112388-149112453 (+) | 22 | CUCCUGACUCCAGGUCCUGUGU | 366 |
| star_st | | | | | |
| hsa-miR-378_st | 0.112811797 | 5:149112388-149112453 (+) | 21 | ACUGGACUUGGAGUCAGAAGG | 367 |
| hsa-miR-379_st | 0.292361055 | 14:101488403-101488469 (+) | 21 | UGGUAGACUAUGGAACGUAGG | 368 |
| hsa-miR-379- | −0.299060613 | 14:101488403-101488469 (+) | 22 | UAUGUAACAUGGUCCACUAACU | 369 |
| star_st | | | | | |
| hsa-miR-380- | −0.260339983 | 14:101491354-101491414 (+) | 22 | UGGUUGACCAUAGAACAUGCGC | 370 |
| star_st | | | | | |
| hsa-miR-380_st | −0.434704478 | 14:101491354-101491414 (+) | 22 | UAUGUAAUAUGGUCCACAUCUU | 371 |
| hsa-miR-381_st | 0.245912087 | 14:101512257-101512331 (+) | 22 | UAUACAAGGGCAAGCUCUCUGU | 372 |
| hsa-miR-382_st | 0.065303359 | 14:101520643-101520718 (+) | 22 | GAAGUUGUUCGUGGUGGAUUCG | 373 |
| hsa-miR-383_st | −0.117482093 | 8:14710947-14711019 (−) | 22 | AGAUCAGAAGGUGAUUGUGGCU | 374 |
| hsa-miR-340_st | 1.486610867 | 5:179442303-179442397 (−) | 22 | UUAUAAAGCAAUGAGACUGAUU | 375 |
| hsa-miR-340- | 0.374825831 | 5:179442303-179442397 (−) | 22 | UCCGUCUCAGUUACUUUAUAGC | 376 |
| star_st | | | | | |
| hsa-miR-330- | 0.16929264 | 19:46142252-46142345 (−) | 22 | UCUCUGGGCCUGUGUCUUAGGC | 377 |
| 5p_st | | | | | |
| hsa-miR-330- | 0.251884038 | 19:46142252-46142345 (−) | 23 | GCAAAGCACACGGCCUGCAGAGA | 378 |
| 3p_st | | | | | |
| hsa-miR-328_st | −0.545026259 | 16:67236224-67236298 (−) | 22 | CUGGCCCUCUCUGCCCUUCCGU | 379 |
| hsa-miR-342- | −0.082156952 | 14:100575992-100576090 (+) | 21 | AGGGGUGCUAUCUGUGAUUGA | 380 |
| 5p_st | | | | | |
| hsa-miR-342- | 0.565853501 | 14:100575992-100576090 (+) | 23 | UCUCACACAGAAAUCGCACCCGU | 381 |
| 3p_st | | | | | |
| hsa-miR-337- | 0.042107983 | 14:101340830-101340922 (+) | 21 | GAACGGCUUCAUACAGGAGUU | 382 |
| 5p_st | | | | | |
| hsa-miR-337- | 0.158376642 | 14:101340830-101340922 (+) | 22 | CUCCUAUAUGAUGCCUUUCUUC | 383 |
| 3p_st | | | | | |
| hsa-miR-323- | −0.511193887 | 14:101492069-101492154 (+) | 22 | AGGUGGUCCGUGGCGCGUUCGC | 384 |
| 5p_st | | | | | |
| hsa-miR-323- | 0.620363762 | 14:101492069-101492154 (+) | 21 | CACAUUACACGGUCGACCUCU | 385 |
| 3p_st | | | | | |
| hsa-miR-326_st | −0.405799761 | 11:75046136-75046230 (−) | 20 | CCUCUGGGCCCUUCCUCCAG | 386 |
| hsa-miR-151- | 0.398889432 | 8:141742663-141742732 (−) | 21 | UCGAGGAGCUCACAGUCUAGU | 387 |
| 5p_st | | | | | |
| hsa-miR-151- | 0.774618536 | 8:141742663-141742752 (−) | 21 | CUAGACUGAAGCUCCUUGAGG | 388 |
| 3p_st | | | | | |
| hsa-miR-135b_st | 0.732231129 | 1:205417430-205417526 (−) | 23 | UAUGGCUUUUCAUUCCUAUGUGA | 389 |
| hsa-miR-135b- | 0.014973154 | 1:205417430-205417526 (−) | 22 | AUGUAGGGCUAAAAGCCAUGGG | 390 |
| star_st | | | | | |
| hsa-miR-148b- | 0.420399734 | 12:54731000-54731098 (+) | 22 | AAGUUCUGUUAUACACUCAGGC | 391 |
| star_st | | | | | |
| hsa-miR-148b_st | 0.284484042 | 12:54731000-54731098 (+) | 22 | UCAGUGCAUCACAGAACUUUGU | 392 |
| hsa-miR-331- | 0.296569348 | 12:95702196-95702289 (+) | 22 | CUAGGUAUGGUCCCAGGGAUCC | 393 |
| 5p_st | | | | | |
| hsa-miR-331- | 0.422330212 | 12:95702196-95702289 (+) | 21 | GCCCCUGGGCCUAUCCUAGAA | 394 |
| 3p_st | | | | | |
| hsa-miR-324- | 0.572325136 | 17:1126616-7126698 (−) | 23 | CGCAUCCCCUAGGGCAUUGGUGU | 395 |
| 5p_st | | | | | |
| hsa-miR-324- | −0.000611543 | 17:7126616-7126698 (−) | 20 | ACUGCCCCAGGUGCUGCUGG | 396 |
| 3p_st | | | | | |
| hsa-miR-338- | 0.933765272 | 17:79099683-79099749 (−) | 22 | AACAAUAUCCUGGUGCUGAGUG | 397 |
| 5p_st | | | | | |
| hsa-miR-338- | 0.921533511 | 17:79099683-79099749 (−) | 22 | UCCAGCAUCAGUGAUUUUGUUG | 398 |
| 3p_st | | | | | |
| hsa-miR-339- | 0.128816497 | 7:1062569-1062662 (−) | 23 | UCCCUGUCCUCCAGGAGCUCACG | 399 |
| 5p_st | | | | | |
| hsa-miR-339- | 0.096359142 | 7:1062569-1062662 (−) | 23 | UGAGCGCCUCGACGACAGAGCCG | 400 |
| 3p_st | | | | | |
| hsa-miR-335_st | 1.288082885 | 7:130135952-130136045 (+) | 23 | UCAAGAGCAAUAACGAAAAAUGU | 401 |
| hsa-miR-335- | 0.2421496 | 7:130135952-130136045 (+) | 22 | UUUUUCAUUAUUGCUCCUGACC | 402 |
| star_st | | | | | |
| hsa-miR-133b_st | −0.444246115 | 6:52013721-52013839 (+) | 22 | UUUGGUCCCCUUCAACCAGCUA | 403 |
| hsa-miR-325_st | 0.13184687 | X:76225829-76225926 (−) | 23 | CCUAGUAGGUGUCCAGUAAGUGU | 404 |
| hsa-miR-345_st | −0.37213779 | 14:100774196-100774293 (+) | 22 | GCUGACUCCUAGUCCAGGGCUC | 405 |
| hsa-miR-346_st | −0.737055304 | 10:88024451-88024545 (−) | 23 | UGUCUGCCCGCAUGCCUGCCUCU | 406 |
| hsa-miR-384_st | 0.043552522 | X:76139698-76139785 (−) | 20 | AUUCCUAGAAAUUGUUCAUA | 407 |
| hsa-miR-196b_st | 0.246365454 | 7:27209099-27209182 (−) | 22 | UAGGUAGUUUCCUGUUGUUGGG | 408 |
| hsa-miR-196b- | −0.01514933 | 7:27209099-27209182 (−) | 22 | UCGACAGCACGACACUGCCUUC | 409 |
| star_st | | | | | |
| hsa-miR-422a_st | −0.18621341 | 15:64163129-64163218 (+) | 22 | ACUGGACUUAGGGUCAGAAGGC | 410 |
| hsa-miR-423- | −0.448953283 | 17:28444097-28444190 (+) | 23 | UGAGGGGCAGAGAGCGAGACUUU | 411 |
| 5p_st | | | | | |
| hsa-miR-423- | 0.070589781 | 17:28444097-28444190 (+) | 23 | AGCUCGGUCUGAGGCCCCUCAGU | 412 |
| 3p_st | | | | | |
| hsa-miR-424_st | 1.385148397 | X:133680644-133680741 (−) | 22 | CAGCAGCAAUUCAUGUUUUGAA | 413 |
| hsa-miR-424- | −0.144490578 | X:133680644-133680741 (−) | 21 | CAAAACGUGAGGCGCUGCUAU | 414 |
| star_st | | | | | |
| hsa-miR-425_st | 1.324811462 | 3:49057581-49057667 (−) | 23 | AAUGACACGAUCACUCCCGUUGA | 415 |
| hsa-miR-425- | −0.039392004 | 3:49057581-49057667 (−) | 22 | AUCGGGAAUGUCGUGUCCGCCC | 416 |
| star_st | | | | | |
| hsa-miR-18b_st | 0.346341595 | X:133304071-133304141 (−) | 23 | UAAGGUGCAUCUAGUGCAGUUAG | 417 |
| hsa-miR-18b- | 0.303689617 | X:133304071-133304141 (−) | 22 | UGCCCUAAAUGCCCCUUCUGGC | 418 |
| star_st | | | | | |
| hsa-miR-20b_st | 0.70973838 | X:133303839-133303907 (−) | 23 | CAAAGUGCUCAUAGUGCAGGUAG | 419 |
| hsa-miR-20b- | 0.065147473 | X:133303839-133303907 (−) | 22 | ACUGUAGUAUGGGCACUUCCAG | 420 |
| star_st | | | | | |
| hsa-miR-448_st | 0.106285981 | X:114058017-114058127 (+) | 22 | UUGCAUAUGUAGGAUGUCCCAU | 421 |
| hsa-miR-429_st | 0.996620656 | 1:1104385-1104467 (+) | 22 | UAAUACUGUCUGGUAAAACCGU | 422 |
| hsa-miR-449a_st | 0.166470516 | 5:54466360-54466450 (−) | 22 | UGGCAGUGUAUUGUUAGCUGGU | 423 |
| hsa-miR-450a_st | 0.773772533 | | 22 | UUUUGCGAUGUGUUCCUAAUAU | 424 |
| hsa-miR-431_st | 0.453467095 | 14:101347344-101347457 (+) | 21 | UGUCUUGCAGGCCGUCAUGCA | 425 |
| hsa-miR-431- | 0.052895261 | 14:101347344-101347457 (+) | 22 | CAGGUCGUCUUGCAGGGCUUCU | 426 |
| star_st | | | | | |
| hsa-miR-433_st | 0.414891105 | 14:101348223-101348315 (+) | 22 | AUCAUGAUGGGCUCCUCGGUGU | 427 |
| hsa-miR-329_st | 1.13163936 | | 22 | AACACACCUGGUUAACCUCUUU | 428 |
| hsa-miR-451_st | −0.530258722 | 17:27188387-27188458 (−) | 22 | AAACCGUUACCAUUACUGAGUU | 429 |
| hsa-miR-451_st | 0.766179813 | X:151128100-151128184 (−) | 22 | AACUGUUUGCAGAGGAAACUGA | 430 |
| hsa-miR-452- | −0.435753634 | X:151128100-151128184 (−) | 22 | CUCAUCUGCAAAGAAGUAAGUG | 431 |
| star_st | | | | | |
| hsa-miR-409- | 0.293542845 | 14:101531637-101531715 (+) | 23 | AGGUUACCCGAGCAACUUUGCAU | 432 |
| 5p_st | | | | | |
| hsa-miR-409- | 0.439001074 | 14:101531637-101531715 (+) | 22 | GAAUGUUGCUCGGUGAACCCCU | 433 |
| 3p_st | | | | | |
| hsa-miR-412_st | 0.156432244 | 14:101531784-101531874 (+) | 23 | ACUUCACCUGGUCCACUAGCCGU | 434 |
| hsa-miR-410_st | 0.384283979 | 14:101532249-101532328 (+) | 21 | AAUAUAACACAGAUGGCCUGU | 435 |
| hsa-miR-376b_st | 0.630717733 | 14:101506773-101506872 (+) | 22 | AUCAUAGAGGAAAAUCCAUGUU | 436 |
| hsa-miR-483- | −0.343228504 | 11:2155364-2155439 (−) | 22 | AAGACGGGAGGAAAGAAGGGAG | 437 |
| 5p_st | | | | | |
| hsa-miR-483- | −0.394618571 | 11:2155364-2155439 (−) | 21 | UCACUCCUCUCCUCCCGUCUU | 438 |
| 3p_st | | | | | |
| hsa-miR-484_st | 0.222490023 | 16:15737151-15737229 (+) | 22 | UCAGGCUCAGUCCCCUCCCGAU | 439 |
| hsa-miR-485- | 0.033075581 | 14:101521756-101521828 (+) | 22 | AGAGGCUGGCCGUGAUGAAUUC | 440 |
| 5p_st | | | | | |
| hsa-miR-485- | 0.042417238 | 14:101521756-401521828 (+) | 22 | GUCAUACACGGCUCUCCUCUCU | 441 |
| 3p_st | | | | | |
| hsa-miR-486- | −1.137032125 | 8:41517959-41518026 (−) | 22 | UCCUGUACUGAGCUGCCCCGAG | 442 |
| 5p_st | | | | | |
| hsa-miR-486- | −0.836193359 | 8:41517959-41518026 (−) | 21 | CGGGGCAGCUCAGUACAGGAU | 443 |
| 3p_st | | | | | |
| hsa-miR-487a_st | 0.043528534 | 14:101518783-101518862 (+) | 22 | AAUCAUACAGGGACAUCCAGUU | 444 |
| hsa-miR-488- | −0.005958782 | 1:176998499-176998581 (−) | 21 | CCCAGAUAAUGGCACUCUCAA | 445 |
| star_st | | | | | |
| hsa-miR-488_st | 0.157128988 | 1:176998499-176998581 (−) | 21 | UUGAAAGGCUAUUUCUUGGUC | 446 |
| hsa-miR-489_st | 0.107045871 | 7:93113248-93113331 (−) | 22 | GUGACAUCACAUAUACGGCAGC | 447 |
| hsa-miR-490- | −0.20048518 | 7:136587914-136588041 (+) | 20 | CCAUGGAUCUCCAGGUGGGU | 448 |
| 5p_st | | | | | |
| hsa-miR-490- | 0.267630951 | 7:136587914-136588041 (+) | 22 | CAACCUGGAGGACUCCAUGCUG | 449 |
| 3p_st | | | | | |
| hsa-miR-491- | −0.346382248 | 9:20716104-20716187 (+) | 22 | AGUGGGGAACCCUUCCAUGAGG | 450 |
| 5p_st | | | | | |
| hsa-miR-491- | −0.061038773 | 9:20716104-20716187 (+) | 22 | CUUAUGCAAGAUUCCCUUCUAC | 451 |
| 3p_st | | | | | |
| hsa-miR-511_st | 0.133854408 | | 21 | GUGUCUUUUGCUCUGCAGUCA | 452 |
| hsa-miR-146b- | 3.996183002 | 10:104196269-104196341 (+) | 22 | UGAGAACUGAAUUCCAUAGGCU | 453 |
| 5p_st | | | | | |
| hsa-miR-146b- | 2.20879991 | 10:104196269-104196341 (+) | 22 | UGCCCUGUGGACUCAGUUCUGG | 454 |
| 3p_st | | | | | |
| hsa-miR-202- | 0.773543112 | 10:135061015-135061124 (−) | 22 | UUCCUAUGCAUAUACUUCUUUG | 455 |
| star_st | | | | | |
| hsa-miR-202_st | −0.25026034 | 10:135061015-135061124 (−) | 20 | AGAGGUAUAGGGCAUGGGAA | 456 |
| hsa-miR-492_st | −0.057616195 | 12:95228174-95228289 (+) | 23 | AGGACCUGCGGGACAAGAUUCUU | 457 |
| hsa-miR-493- | 1.16919763 | 14:101335397-101335485 (+) | 22 | UUGUACAUGGUAGGCUUUCAUU | 458 |
| star_st | | | | | |
| hsa-miR-493_st | −0.018931488 | 14:101335397-101335485 (+) | 22 | UGAAGGUCUACUGUGUGCCAGG | 459 |
| hsa-miR-432_st | −0.01535762 | 14:101350820-101350913 (+) | 23 | UCUUGGAGUAGGUCAUUGGGUGG | 460 |
| hsa-miR-432- | −0.069305014 | 14:101350820-101350913 (+) | 21 | CUGGAUGGCUCCUCCAUGUCU | 461 |
| star_st | | | | | |
| hsa-miR-494_st | 0.040111825 | 14:101495971-101496051 (+) | 22 | UGAAACAUACACGGGAAACCUC | 462 |
| hsa-miR-495_st | 0.999893612 | 14:101500092-101500173 (+) | 22 | AAACAAACAUGGUGCACUUCUU | 463 |
| hsa-miR-496_st | −0.073853056 | 14:101526910-101527011 (+) | 22 | UGAGUAUUACAUGGCCAAUCUC | 464 |
| hsa-miR-193b- | −0.749675934 | 16:14397824-14397906 (+) | 22 | CGGGGUUUUGAGGGCGAGAUGA | 465 |
| star_st | | | | | |
| hsa-miR-193b_st | 0.34755861 | 16:14397824-14397906 (+) | 22 | AACUGGCCCUCAAAGUCCCGCU | 466 |
| hsa-miR-497_st | 0.134042327 | 17:6921230-6921341 (−) | 21 | CAGCAGCACACUGUGGUUUGU | 467 |
| hsa-miR-497- | −0.449382958 | 17:6921230-6921341 (−) | 22 | CAAACCACACUGUGGUGUUAGA | 468 |
| star_st | | | | | |
| hsa-miR-181d_st | 0.279397009 | 19:13985689-13985825 (+) | 23 | AACAUUCAUUGUUGUCGGUGGGU | 469 |
| hsa-miR-512- | −0.112782951 | | 23 | CACUCAGCCUUGAGGGCACUUUC | 470 |
| 5p_st | | | | | |
| hsa-miR-512- | −0.375147916 | | 22 | AAGUGCUGUCAUAGCUGAGGUC | 471 |
| 3p_st | | | | | |
| hsa-miR-498_st | −0.547531217 | 19:54177451-54177574 (+) | 23 | UUUCAAGCCAGGGGGCGUUUUUC | 472 |
| hsa-miR-520e_st | 0.482212017 | 19:54178965-54179051 (+) | 21 | AAAGUGCUUCCUUUUUGAGGG | 473 |
| hsa-miR-515- | 0.423256921 | | 24 | UUCUCCAAAAGAAAGCACUUUCUG | 474 |
| 5p_st | | | | | |
| hsa-miR-515- | 0.35494522 | | 22 | GAGUGCCUUCUUUUGGAGCGUU | 475 |
| 3p_st | | | | | |
| hsa-miR-519e- | −0.130694532 | 19:54183194-54183277 (+) | 22 | UUCUCCAAAAGGGAGCACUUUC | 476 |
| star_st | | | | | |
| hsa-miR-519e_st | 0.080062506 | 19:54183194-54183277 (+) | 22 | AAGUGCCUCCUUUUAGAGUGUU | 477 |
| hsa-miR-520f_st | 0.559392629 | 19:54185413-54185499 (+) | 22 | AAGUGCUUCCUUUUAGAGGGUU | 478 |
| hsa-miR-519c- | −0.306221622 | 19:54189723-54189809 (+) | 22 | CUCUAGAGGGAAGCGCUUUCUG | 479 |
| 5p_st | | | | | |
| hsa-miR-519c- | −0.343409838 | 19:54189723-54189809 (+) | 22 | AAAGUGCAUCUUUUUAGAGGAU | 480 |
| 3p_st | | | | | |
| hsa-miR-520a- | −0.304054616 | 19:54194135-54194219 (+) | 21 | CUCCAGAGGGAAGUACUUUCU | 481 |
| 5p_st | | | | | |
| hsa-miR-520a- | 0.098785541 | 19:54194135-54194219 (+) | 22 | AAAGUGCUUCCCUUUGGACUGU | 482 |
| 3p_st | | | | | |
| hsa-miR-526b_st | −0.421250558 | 19:54197647-54197729 (+) | 23 | CUCUUGAGGGAAGCACUUUCUGU | 483 |
| hsa-miR-526b- | 0.32377889 | 19:54197647-54197729 (+) | 22 | GAAAGUGCUUCCUUUUAGAGGC | 484 |
| star_st | | | | | |
| hsa-miR-519b- | −0.331347859 | 19:54198467-54198547 (+) | 22 | CUCUAGAGGGAAGCGCUUUCUG | 485 |
| 5p_st | | | | | |
| hsa-miR-519b- | −0.232036923 | 19:54198467-54198547 (+) | 22 | AAAGUGCAUCCUUUUAGAGGUU | 486 |
| 3p_st | | | | | |
| hsa-miR-525- | −0.312719533 | 19:54200787-54200871 (+) | 21 | CUCCAGAGGGAUGCACUUUCU | 487 |
| 5p_st | | | | | |
| hsa-miR-525- | −0.267350286 | 19:54200787-54200871 (+) | 22 | GAAGGCGCUUCCCUUUAGAGCG | 488 |
| 3p_st | | | | | |
| hsa-miR-523- | −0.672353045 | 19:54201639-54201725 (+) | 22 | CUCUAGAGGGAAGCGCUUUCUG | 489 |
| star_st | | | | | |
| hsa-miR-523_st | −0.04008021 | 19:54201639-54201725 (+) | 23 | GAACGCGCUUCCCUAUAGAGGGU | 490 |
| hsa-miR-518f- | −0.0545996 | 19:54203269-54203355 (+) | 22 | CUCUAGAGGGAAGCACUUUCUC | 491 |
| star_st | | | | | |
| hsa-miR-518f_st | −0.014742339 | 19:54203269-54203355 (+) | 21 | GAAAGCGCUUCUCUUUAGAGG | 492 |
| hsa-miR-520b_st | −0.189647326 | 19:54204481-54204541 (+) | 21 | AAAGUGCUUCCUUUUAGAGGG | 493 |
| hsa-miR-518b_st | −0.558223555 | 19:54205991-54206073 (+) | 22 | CAAAGCGCUCCCCUUUAGAGGU | 494 |
| hsa-miR-526a_st | −0.218681766 | | 22 | CUCUAGAGGGAAGCACUUUCUG | 495 |
| hsa-miR-520c- | 0.568307623 | 19:54210707-54210793 (+) | 22 | CUCUAGAGGGAAGCACUUUCUG | 496 |
| 5p_st | | | | | |
| hsa-miR-520c- | 0.062702555 | 19:54210707-54210793 (+) | 22 | AAAGUGCUUCCUUUUAGAGGGU | 497 |
| 3p_st | | | | | |
| hsa-miR-518c- | −0.45454021 | 19:54211989-54212089 (+) | 23 | UCUCUGGAGGGAAGCACUUUCUG | 498 |
| star_st | | | | | |
| hsa-miR-518c_st | −0.08750365 | 19:54211989-54212089 (+) | 23 | CAAAGCGCUUCUCUUUAGAGUGU | 499 |
| hsa-miR-524- | 0.045430946 | 19:54214256-54214342 (+) | 22 | CUACAAAGGGAAGCACUUUCUC | 500 |
| 5p_st | | | | | |
| hsa-miR-524- | −0.375242902 | 19:54214256-54214342 (+) | 21 | GAAGGCGCUUCCCUUUGGAGU | 501 |
| 3p_st | | | | | |
| hsa-miR-517- | −0.199841722 | | 22 | CCUCUAGAUGGAAGCACUGUCU | 502 |
| star_st | | | | | |
| hsa-miR-517a_st | −0.740187506 | 19:54215522-54215608 (+) | 22 | AUCGUGCAUCCCUUUAGAGUGU | 503 |
| hsa-miR-519d_st | −0.028025737 | 19:54216601-54216688 (+) | 22 | CAAAGUGCCUCCCUUUAGAGUG | 504 |
| hsa-miR-521_st | 0.032607535 | | 22 | AACGCACUUCCCUUUAGAGUGU | 505 |
| hsa-miR-520d- | 0.026642305 | 19:54223350-54223436 (+) | 20 | CUACAAAGGGAAGCCCUUUC | 506 |
| 5p_st | | | | | |
| hsa-miR-520d- | 0.406772017 | 19:54223350-54223436 (+) | 22 | AAAGUGCUUCUCUUUGGUGGGU | 507 |
| 3p_st | | | | | |
| hsa-miR-517b_st | −0.229573511 | 19:54224330-54224396 (+) | 22 | UCGUGCAUCCCUUUAGAGUGUU | 508 |
| hsa-miR-520g_st | −0.312270335 | 19:54225420-54225509 (+) | 24 | ACAAAGUGCUUCCCUUUAGAGUGU | 509 |
| hsa-miR-516b_st | −0.5224054 | | 22 | AUCUGGAGGUAAGAAGCACUUU | 510 |
| hsa-miR-516b- | 0.221707991 | | 18 | UGCUUCCUUUCAGAGGGU | 511 |
| star_st | | | | | |
| hsa-miR-518c- | −0.227533992 | 19:54233092-54233179 (+) | 22 | CUCUAGAGGGAAGCGCUUUCUG | 512 |
| star_st | | | | | |
| hsa-miR-518c_st | −0.414617824 | 19:54233092-54233179 (+) | 21 | AAAGCGCUUCCCUUCAGAGUG | 513 |
| hsa-miR-518a- | 0.007114621 | | 20 | CUGCAAAGGGAAGCCCUUUC | 514 |
| 5p_st | | | | | |
| hsa-miR-518a- | −0.11579863 | | 22 | GAAAGCGCUUCCCUUUGCUGGA | 515 |
| 3p_st | | | | | |
| hsa-miR-518d- | −0.005453447 | 19:54238131-54238217 (+) | 22 | CUCUAGAGGGAAGCACUUUCUG | 516 |
| 5p_st | | | | | |
| hsa-miR-518d- | 0.53069303 | 19:54238131-54238217 (+) | 21 | CAAAGCGCUUCCCUUUGGAGC | 517 |
| 3p_st | | | | | |
| hsa-miR-517c_st | −0.468801766 | 19:54244567-54244661 (+) | 22 | AUCGUGCAUCCUUUUAGAGUGU | 518 |
| hsa-miR-520h_st | −0.535245541 | 19:54245766-54245853 (+) | 22 | ACAAAGUGCUUCCCUUUAGAGU | 519 |
| hsa-miR-522_ | −0.041532652 | 19:54254465-54254551 (+) | 22 | CUCUAGAGGGAAGCGCUUUCUG | 520 |
| star_st | | | | | |
| hsa-miR-522_st | −0.582708908 | 19:54254465-54254551 (+) | 22 | AAAAUGGUUCCCUUUAGAGUGU | 521 |
| hsa-miR-519a- | −0.476655144 | | 22 | CUCUAGAGGGAAGCGCUUUCUG | 522 |
| star_st | | | | | |
| hsa-miR-519a_st | −1.207470298 | | 22 | AAAGUGCAUCCUUUUAGAGUGU | 523 |
| hsa-miR-527_st | 0.06899744 | 19:54257272-54257356 (+) | 20 | CUGCAAAGGGAAGCCCUUUC | 524 |
| hsa-miR-516a- | −0.164926747 | | 23 | UUCUCGAGGAAAGAAGCACUUUC | 525 |
| 5p_st | | | | | |
| hsa-miR-516a- | 0.132099039 | | 18 | UGCUUCCUUUCAGAGGGU | 526 |
| 3p_st | | | | | |
| hsa-miR-499- | 0.749726599 | 20:33578179-33578300 (+) | 21 | UUAAGACUUGCAGUGAUGUUU | 527 |
| 5p_st | | | | | |
| hsa-miR-499- | 0.377865059 | 20:33578179-33578300 (+) | 22 | AACAUCACAGCAAGUCUGUGCU | 528 |
| 3p_st | | | | | |
| hsa-miR-500_st | 0.438632739 | X:49773039-49773122 (+) | 23 | UAAUCCUUGCUACCUGGGUGAGA | 529 |
| hsa-miR-500- | −0.42456855 | X:49773039-49773122 (+) | 22 | AUGCACCUGGGCAAGGAUUCUG | 530 |
| star_st | | | | | |
| hsa-miR-501- | −0.135102225 | X:49774330-49774413 (+) | 22 | AAUCCUUUGUCCCUGGGUGAGA | 531 |
| 5p_st | | | | | |
| hsa-miR-501- | 0.101944124 | X:49774330-49774413 (+) | 22 | AAUGCACCCGGGCAAGGAUUCU | 532 |
| 3p_st | | | | | |
| hsa-miR-502- | −0.026150917 | X:49779206-49779291 (+) | 21 | AUCCUUGCUAUCUGGGUGCUA | 533 |
| 5p_st | | | | | |
| hsa-miR-502- | 0.050161315 | X:49779206-49779291 (+) | 22 | AAUGCACCUGGGCAAGGAUUCA | 534 |
| 3p_st | | | | | |
| hsa-miR-503_st | −0.323947388 | X:133680358-133680428 (−) | 23 | UAGCAGCGGGAACAGUUCUGCAG | 535 |
| hsa-miR-504_st | −0.042496164 | X:137749872-137749954 (−) | 22 | AGACCCUGGUCUGCACUCUAUC | 536 |
| hsa-miR-505- | −0.236339932 | X:139006307-139006390 (−) | 22 | GGGAGCCAGGAAGUAUUGAUGU | 537 |
| star_st | | | | | |
| hsa-miR-505_st | 0.547035344 | X:139006307-139006390 (−) | 22 | CGUCAACACUUGCUGGUUUCCU | 538 |
| hsa-miR-513a- | −0.170439794 | | 18 | UUCACAGGGAGGUGUCAU | 539 |
| 5p_st | | | | | |
| hsa-miR-513a- | 0.021131387 | | 23 | UAAAUUUCACCUUUCUGAGAAGG | 540 |
| 3p_st | | | | | |
| hsa-miR-506_st | −0.035103939 | X:146312238-146312361 (−) | 21 | UAAGGCACCCUUCUGAGUAGA | 541 |
| hsa-miR-507_st | −0.161893667 | X:146312502-146312595 (−) | 21 | UUUUGCACCUUUUGGAGUGAA | 542 |
| hsa-miR-508- | −0.403192169 | X:146318431-146318545 (−) | 23 | UACUCCAGAGGGCGUCACUCAUG | 543 |
| 5p_st | | | | | |
| hsa-miR-508- | 0.600164117 | X:146318431-146318545 (−) | 23 | UGAUUGUAGCCUUUUGGAGUAGA | 544 |
| 3p_st | | | | | |
| hsa-miR-509- | 0.098605249 | | 21 | UACUGCAGACAGUGGCAAUCA | 545 |
| 5p_st | | | | | |
| hsa-miR-509- | 0.048840256 | | 22 | UGAUUGGUACGUCUGUGGGUAG | 546 |
| 3p_st | | | | | |
| hsa-miR-510_st | −0.25440859 | X:146333853-146353926 (−) | 22 | UACUCAGGAGAGUGGCAAUCAC | 547 |
| hsa-miR-514_st | −0.035560896 | | 21 | AUUGACACUUCUGUGAGUAGA | 548 |
| hsa-miR-532- | −0.104257757 | X:49767754-49767844 (+) | 22 | CAUGCCUUGAGUGUAGGACCGU | 549 |
| 5p_st | | | | | |
| hsa-miR-532- | −0.268605096 | X:49767754-49767844 (+) | 22 | CCUCCCACACCCAAGGCUUGCA | 550 |
| 3p_st | | | | | |
| hsa-miR-455- | 0.337768864 | 9:116971714-116971809 (+) | 22 | UAUGUGCCUUUGGACUACAUCG | 551 |
| 5p_st | | | | | |
| hsa-miR-455- | −0.07615287 | 9:116971714-116971809 (+) | 21 | GCAGUCCAUGGGCAUAUACAC | 552 |
| 3p_st | | | | | |
| hsa-miR-539_st | 0.687043185 | 14:101513658-101513735 (+) | 22 | GGAGAAAUUAUCCUUGGUGUGU | 553 |
| hsa-miR-544_st | −0.05428565 | 14:101514995-101515085 (+) | 22 | AUUCUGCAUUUUUAGCAAGUUC | 554 |
| hsa-miR-545- | 0.207542716 | X:73506939-73507044 (−) | 22 | UCAGUAAAUGUUUAUUAGAUGA | 555 |
| star_st | | | | | |
| hsa-miR-545_st | −0.098963665 | X:73506939-73507044 (−) | 22 | UCAGCAAACAUUUAUUGUGUGC | 556 |
| hsa-miR-487b_st | 0.956196746 | 14:101512792-101512875 (+) | 22 | AAUCGUACAGGGUCAUCCACUU | 557 |
| hsa-miR-551a_st | 0.227567501 | 1:3477259-3477354 (−) | 21 | GCGACCCACUCUUGGUUUCCA | 558 |
| hsa-miR-552_st | 0.23364223 | 1:35135200-35135295 (−) | 21 | AACAGGUGACUGGUUAGACAA | 559 |
| hsa-miR-553_st | −0.147296893 | 1:100746797-100746864 (+) | 21 | AAAACGGUGAGAUUUUGUUUU | 560 |
| hsa-miR-554_st | −0.011540398 | 1:151518272-151518367 (+) | 21 | GCUAGUCCUGACUCAGCCAGU | 561 |
| hsa-miR-92b- | −0.542496243 | 1:155164968-155165063 (+) | 22 | AGGGACGGGACGCGGUGCAGUG | 562 |
| star_st | | | | | |
| hsa-miR-92b_st | −0.14926178 | 1:155164968-155165063 (+) | 22 | UAUUGCACUCGUCCCGGCCUCC | 563 |
| hsa-miR-555_st | 0.0865631 | 1:155316141-155316236 (−) | 21 | AGGGUAAGCUGAACCUCUGAU | 564 |
| hsa-miR-556- | 0.065353696 | 1:162312336-162312430 (+) | 22 | GAUGAGCUCAUUGUAAUAUGAG | 565 |
| 5p_st | | | | | |
| hsa-miR-556- | 0.48368382 | 1:162312336-162312430 (+) | 22 | AUAUUACCAUUAGCUCAUCUUU | 566 |
| 3p_st | | | | | |
| hsa-miR-557_st | −0.848077223 | 1:168344762-168344859 (+) | 23 | GUUUGCACGGGUGGGCCUUGUCU | 567 |
| hsa-miR-558_st | 0.570822562 | 2:32757220-32757313 (+) | 19 | UGAGCUGCUGUACCAAAAU | 568 |
| hsa-miR-559_st | 0.371533619 | 2:47604814-47604909 (+) | 21 | UAAAGUAAAUAUGCACCAAAA | 569 |
| hsa-miR-561_st | 0.298993037 | 2:189162219-189162315 (+) | 22 | CAAAGUUUAAGAUCCUUGAAGU | 570 |
| hsa-miR-562_st | −0.07259205 | 2:233037363-233037457 (+) | 20 | AAAGUAGCUGUACCAUUUGC | 571 |
| hsa-miR-563_st | 0.199429719 | 3:15915278-15915356 (+) | 19 | AGGUUGACAUACGUUUCCC | 572 |
| hsa-miR-564_st | −0.2969312 | 3:44903380-44903473 (+) | 19 | AGGCACGGUGUCAGCAGGC | 573 |
| hsa-miR-566_st | −0.238839377 | 3:50210759-50210852 (+) | 19 | GGGCGCCUGUGAUCCCAAC | 574 |
| hsa-miR-567_st | −0.436437282 | 3:111831648-111831745 (+) | 23 | AGUAUGUUCUUCCAGGACAGAAC | 575 |
| hsa-miR-568_st | −0.194443772 | 3:114035322-114033416 (−) | 20 | AUGUAUAAAUGUAUACACAC | 576 |
| hsa-miR-551b- | −0.024490803 | 3:168269642-168269737 (+) | 22 | GAAAUCAAGCGUGGGUGAGACC | 577 |
| star_st | | | | | |
| hsa-miR-551b_st | 1.886743732 | 3:168269642-168269737 (+) | 21 | GCGACCCAUACUUGGUUUCAG | 578 |
| hsa-miR-569_st | −0.223246676 | 3:170824453-170824548 (−) | 21 | AGUUAAUGAAUCCUGGAAAGU | 579 |
| hsa-miR-570_st | 0.449888664 | 3:195426272-195426368 (+) | 22 | CGAAAACAGCAAUUACCUUUGC | 580 |
| hsa-miR-571_st | 0.21553837 | 4:343946-344041 (+) | 21 | UGAGUUGGCCAUCUGAGUGAG | 581 |
| hsa-miR-572_st | −0.560537877 | 4:11370451-11370545 (+) | 20 | GUCCGCUCGGCGGUGGCCCA | 582 |
| hsa-miR-573_st | −0.477735488 | 4:24521815-24521913 (−) | 24 | CUGAAGUGAUGUGUAACUGAUCAG | 583 |
| hsa-miR-574- | −0.561164498 | 4:38869653-38869748 (+) | 23 | UGAGUGUGUGUGUGUGAGUGUGU | 584 |
| 5p_st | | | | | |
| hsa-miR-574- | −0.032326635 | 4:38869653-38869748 (+) | 22 | CACGCUCAUGCACACACCCACA | 585 |
| 3p_st | | | | | |
| hsa-miR-575_st | −0.01201755 | 4:83674490-83674583 (−) | 19 | GAGCCAGUUGGACAGGAGC | 586 |
| hsa-miR-576- | 0.095403984 | 4:110409854-110409951 (+) | 22 | AUUCUAAUUUCUCCACGUCUUU | 587 |
| 5p_st | | | | | |
| hsa-miR-576- | 0.003145179 | 4:110409854-110409951 (+) | 22 | AAGAUGUGGAAAAAUUGGAAUC | 588 |
| 3p_st | | | | | |
| hsa-miR-577_st | 0.431296077 | 4:115577915-115578010 (+) | 21 | UAGAUAAAAUAUUGGUACCUG | 589 |
| hsa-miR-578_st | 0.659167305 | 4:166307394-166307489 (+) | 21 | CUUCUUGUGCUCUAGGAUUGU | 590 |
| hsa-miR-579_st | 0.419680008 | 5:32394484-32394581 (−) | 23 | UUCAUUUGGUAUAAACCGCGAUU | 591 |
| hsa-miR-580_st | −0.519250306 | 5:36147994-36148090 (−) | 22 | UUGAGAAUGAUGAAUCAUUAGG | 592 |
| hsa-miR-581_st | −0.360717853 | 5:53247334-53247429 (−) | 21 | UCUUGUGUUCUCUAGAUCAGU | 593 |
| hsa-miR-582- | 0.654531481 | 5:58999432-58999529 (−) | 23 | UUACAGUUGUUCAACCAGUUACU | 594 |
| 5p_st | | | | | |
| hsa-miR-582- | 0.206923235 | 5:58999432-58999529 (−) | 22 | UAACUGGUUGAACAACUGAACC | 595 |
| 3p_st | | | | | |
| hsa-miR-583_st | 0.015713694 | 5:95414842-95414916 (+) | 21 | CAAAGAGGAAGGUCCCAUUAC | 596 |
| hsa-miR-584_st | 0.260079153 | 5:148441876-148441972 (−) | 22 | UUAUGGUUUGCCUGGGACUGAG | 597 |
| hsa-miR-585_st | −0.19802712 | 5:168690605-168690698 (−) | 19 | UGGGCGUAUCUGUAUGCUA | 598 |
| hsa-miR-548a- | 0.05546039 | | 22 | CAAAACUGGCAAUUACUUUUGC | 599 |
| 3p_st | | | | | |
| hsa-miR-586_st | −0.107151837 | 6:45165411-45165507 (−) | 22 | UAUGCAUUGUAUUUUUAGGUCC | 600 |
| hsa-miR-587_st | 0.201686654 | 6:107232000-107232095 (+) | 21 | UUUCCAUAGGUGAUGAGUCAC | 601 |
| hsa-miR-548b- | 0.064324541 | 6:119390212-119390308 (−) | 22 | AAAAGUAAUUGUGGUUUUGGCC | 602 |
| 5p_st | | | | | |
| hsa-miR-548b- | 0.511981078 | 6:119390212-119390308 (−) | 22 | CAAGAACCUCAGUUGCUUUUGU | 603 |
| 3p_st | | | | | |
| hsa-miR-588_st | 0.119934863 | 6:126805777-126805859 (+) | 21 | UUGGCCACAAUGGGUUAGAAC | 604 |
| hsa-miR-589_st | 0.236078491 | 7:5535450-5535548 (−) | 22 | UGAGAACCACGUCUGCUCUGAG | 605 |
| hsa-miR-589- | −0.001139358 | 7:5535450-5535548 (−) | 24 | UCAGAACAAAUGCCGGUUCCCAGA | 606 |
| star_st | | | | | |
| hsa-miR-550_st | −0.195598432 | | 23 | AGUGCCUGAGGGAGUAAGAGCCC | 607 |
| hsa-miR-550- | 0.221295836 | | 23 | UGUCUUACUCCCUCAGGCACAU | 608 |
| star_st | | | | | |
| hsa-miR-590- | 0.16442954 | 7:73605528-73605624 (+) | 22 | GAGCUUAUUCAUAAAAGUGCAG | 609 |
| 5p_st | | | | | |
| hsa-miR-590- | −0.049746069 | 7:73605528-73605624 (+) | 21 | UAAUUUUAUGUAUAAGCUAGU | 610 |
| 3p_st | | | | | |
| hsa-miR-591_st | −0.141851032 | 7:95848974-95849068 (−) | 20 | AGACCAUGGGUUCUCAUUGU | 611 |
| hsa-miR-592_st | 0.322713552 | 7:126698142-126698238 (−) | 22 | UUGGUCAAUAUGCGAUGAUGU | 612 |
| hsa-miR-593- | −0.03399546 | 7:127721913-127722012 (+) | 25 | AGGCACCAGCCAGGCAUUGCUCAGC | 613 |
| star_st | | | | | |
| hsa-miR-593_st | −0.043538192 | 7:127721913-127722012 (+) | 19 | UGUCUCUGCUGGGGUUUCU | 614 |
| hsa-miR-595 st | −0.140019306 | 7:158325410-158325505 (−) | 21 | GAAGUGUGCCGUGGUGUGUCU | 615 |
| hsa-miR-596_st | −0.782494109 | 8:1765397-1765473 (+) | 21 | AAGCCUGCCCGGCUCCUCGGG | 616 |
| hsa-miR-597_st | 0.254557713 | 8:9599182-9599278 (+) | 22 | UGUGUCACUCGAUGACCACUGU | 617 |
| hsa-miR-598_st | 0.719291732 | 8:10892716-10892812 (−) | 22 | UACGUCAUCGUUGUCAUCGUCA | 618 |
| hsa-miR-599_st | 0.341052563 | 8:100548864-100548958 (−) | 20 | GUUGUGUCAGUUUAUCAAAC | 619 |
| hsa-miR-548a- | 0.144268332 | | 22 | AAAAGUAAUUGCGAGUUUUACC | 620 |
| 5p_st | | | | | |
| hsa-miR-600_st | 0.21359635 | 9:125873825-125873922 (−) | 23 | ACUUACAGACAAGAGCCUUGCUC | 621 |
| hsa-miR-601_st | −0.037995238 | 9:126164804-126164882 (−) | 22 | UGGUCUAGGAUUGUUGGAGGAG | 622 |
| hsa-miR-602_st | −0.59149535 | 9:140732871-140732968 (+) | 23 | GACACGGGCGACAGCUGCGGCCC | 623 |
| hsa-miR-603_st | 0.153581012 | 10:24564614-24564710 (+) | 22 | CACACACUGCAAUUACUUUUGC | 624 |
| hsa-miR-604_st | 0.051861448 | 10:29833933-29834026 (−) | 19 | AGGCUGCGGAAUUCAGGAC | 625 |
| hsa-miR-605_st | −0.151798212 | 10:53059333-53059415 (+) | 23 | UAAAUCCCAUGGUGCCUUCUCCU | 626 |
| hsa-miR-606_st | 0.321319999 | 10:77312216-77312311 (+) | 21 | AAACUACUGAAAAUCAAAGAU | 627 |
| hsa-miR-607_st | 0.381590014 | 10:98588426-98588521 (−) | 21 | GUUCAAAUCCAGAUCUAUAAC | 628 |
| hsa-miR-608_st | −0.523042342 | 10:102734742-102734841 (+) | 25 | AGGGGUGGUGUUGGGACAGCUCCGU | 629 |
| hsa-miR-609_st | −0.252595945 | 10:105978547-105978641 (−) | 20 | AGGGUGUUUCUCUCAUCUCU | 630 |
| hsa-miR-610_st | 0.356352246 | 11:28078362-28078457 (+) | 21 | UGAGCUAAAUGUGUGCUGGGA | 631 |
| hsa-miR-611_st | −0.625890778 | 11:61559967-61560033 (−) | 23 | GCGAGGACCCCUCGGGGUCUGAC | 632 |
| hsa-miR-612_st | −0.597505963 | 11:65211929-65212028 (+) | 25 | GCUGGGCAGGGCUUCUGAGCUCCUU | 633 |
| hsa-miR-613_st | 0.129893178 | 12:12917583-12917677 (+) | 20 | AGGAAUGUUCCUUCUUUGCC | 634 |
| hsa-miR-614_st | 0.169744108 | 12:13068763-13068852 (+) | 23 | GAACGCCUGUUCUUGCCAGGUGG | 635 |
| hsa-miR-615- | −0.546348066 | 12:54427734-54427829 (+) | 22 | GGGGGUCCCCGGUGCUCGGAUC | 636 |
| 5p_st | | | | | |
| hsa-miR-615- | −0.558062945 | 12:54427734-54427829 (+) | 22 | UCCGAGCCUGGGUCUCCCUCUU | 637 |
| 3p_st | | | | | |
| hsa-miR-616- | 0.150826863 | 12:57912946-57913042 (−) | 22 | ACUCAAAACCCUUCAGUGACUU | 638 |
| star_st | | | | | |
| hsa-miR-616_st | −0.593150398 | 12:57912946-57913042 (−) | 22 | AGUCAUUGGAGGGUUUGAGCAG | 639 |
| hsa-miR-548c- | 0.20696522 | 12:65016289-65016385 (+) | 22 | AAAAGUAAUUGCGGUUUUUGCC | 640 |
| 5p_st | | | | | |
| hsa-miR-548c- | 0.38824374 | 12:65016289-65016385 (+) | 22 | CAAAAAUCUCAAUUACUUUUGC | 641 |
| 3p_st | | | | | |
| hsa-miR-617_st | 0.179802415 | 12:81226312-81226408 (−) | 22 | AGACUUCCCAUUUGAAGGUGGC | 642 |
| hsa-miR-618_st | 0.301761441 | 12:81329515-81329612 (−) | 23 | AAACUCUACUUGUCCUUCUGAGU | 643 |
| hsa-miR-619_st | 0.05602629 | 12:109230684-109230782 (−) | 24 | GACCUGGACAUGUUUGUGCCCAGU | 644 |
| hsa-miR-620_st | 0.170970712 | 12:116586365-116586459 (−) | 20 | AUGGAGAUAGAUAUAGAAAU | 645 |
| hsa-miR-621_st | −0.133416356 | 13:41384902-41384997 (+) | 21 | GGCUAGCAACAGCGCUUACCU | 646 |
| hsa-miR-622_st | 0.175781108 | 13:90883436-90883531 (+) | 21 | ACAGUCUGCUGAGGUUGGAGC | 647 |
| hsa-miR-623_st | −0.649568833 | 13:100008385-100008482 (+) | 23 | AUCCCUUGCAGGGGCUGUUGGGU | 648 |
| hsa-miR-624- | −0.111181783 | 14:31483852-31483948 (−) | 22 | UAGUACCAGUACCUUGUGUUCA | 649 |
| star_st | | | | | |
| hsa-miR-624_st | −0.470763983 | 14:31483852-31483948 (−) | 21 | CACAAGGUAUUGGUAUUACCU | 650 |
| hsa-miR-625_st | −0.237221077 | 14:65937820-65937904 (+) | 21 | AGGGGGAAAGUUCUAUAGUCC | 651 |
| hsa-miR-625- | −0.04463785 | 14:65937820-65937904 (+) | 22 | GACUAUAGAACUUUCCCCCUCA | 652 |
| star_st | | | | | |
| hsa-miR-626_st | 0.420281913 | 15:41983783-41983876 (+) | 19 | AGCUGUCUGAAAAUGUCUU | 653 |
| hsa-miR-627_st | 0.049034464 | 15:42491768-42491864 (−) | 22 | GUGAGUCUCUAAGAAAAGAGGA | 654 |
| hsa-miR-628- | 0.178537539 | 15:55665138-55665232 (−) | 22 | AUGCUGACAUAUUUACUAGAGG | 655 |
| 5p_st | | | | | |
| hsa-miR-628- | −0.07636519 | 15:55665138-55665232 (−) | 21 | UCUAGUAAGAGUGGCAGUCGA | 656 |
| 3p_st | | | | | |
| hsa-miR-629_st | 0.310237475 | 15:70371711-70371807 (−) | 21 | UGGGUUUACGUUGGGAGAACU | 657 |
| hsa-miR-629- | −0.062387318 | 15:70371711-70371807 (−) | 22 | GUUCUCCCAACGUAAGCCCAGC | 658 |
| star_st | | | | | |
| hsa-miR-630_st | −0.071442144 | 15:72879558-72879654 (+) | 22 | AGUAUUCUGUACCAGGGAAGGU | 659 |
| hsa-miR-631_st | −0.128933028 | 15:75645952-75646026 (−) | 21 | AGACCUGGCCCAGACCUCAGC | 660 |
| hsa-miR-33b_st | −0.149152609 | 17:17717150-17717245 (−) | 20 | GUGCAUUGCUGUUGCAUUGC | 661 |
| hsa-miR-33b- | −0.335924749 | 17:17717150-17717245 (−) | 22 | CAGUGCCUCGGCAGUGCAGCCC | 662 |
| star_st | | | | | |
| hsa-miR-632_st | 0.194451438 | 17:30677128-30677221 (+) | 19 | GUGUCUGCUUCCUGUGGGA | 663 |
| hsa-miR-633_st | 0.194132983 | 17:61021576-61021673 (+) | 23 | CUAAUAGUAUCUACCACAAUAAA | 664 |
| hsa-miR-634_st | 0.016003366 | 17:64783190-64783286 (+) | 22 | AACCAGCACCCCAACUUUGGAC | 665 |
| hsa-miR-635_st | −0.1564536 | 17:66420592-66420689 (−) | 23 | ACUUGGGCACUGAAACAAUGUCC | 666 |
| hsa-miR-636_st | −0.782589366 | 17:74732532-74732630 (−) | 23 | UGUGCUUGCUCGUCCCGCCCGCA | 667 |
| hsa-miR-637_st | −0.697029065 | 19:3961412-3961510 (−) | 24 | ACUGGGGGCUUUCGGGCUCUGCGU | 668 |
| hsa-miR-638_st | −0.422208669 | 19:10829080-10829179 (+) | 25 | AGGGAUCGCGGGCGGGUGGCGGCCU | 669 |
| hsa-miR-639_st | −0.668433943 | 19:14640355-14640452 (+) | 23 | AUCGCUGCGGUUGCGAGCGCUGU | 670 |
| hsa-miR-640_st | −0.248474937 | 19:19545872-19545967 (+) | 21 | AUGAUCCAGGAACCUGCCUCU | 671 |
| hsa-miR-641_st | 0.037050977 | 19:40788450-40788548 (−) | 24 | AAAGACAUAGGAUAGAGUCACCUC | 672 |
| hsa-miR-642_st | 0.494383667 | 19:46178186-46178282 (+) | 22 | GUCCCUCUCCAAAUGUGUCUUG | 673 |
| hsa-miR-643_st | 0.235850589 | 19:52785050-52785146 (+) | 22 | ACUUGUAUGCUAGCUCAGGUAG | 674 |
| hsa-miR-644_st | −0.074050142 | 20:33054130-33054223 (+) | 19 | AGUGUGGCUUUCUUAGAGC | 675 |
| hsa-miR-645_st | 0.160387161 | 20:49202323-49202416 (+) | 19 | UCUAGGCUGGUACUGCUGA | 676 |
| hsa-miR-646_st | 0.051778024 | 20:58883532-58883625 (+) | 19 | AAGCAGCUGCCUCUGAGGC | 677 |
| hsa-miR-647_st | −0.305580715 | 20:62573984-62574079 (−) | 21 | GUGGCUGCACUCACUUCCUUC | 678 |
| hsa-miR-648_st | −0.349328299 | 22:18463634-18463727 (−) | 19 | AAGUGUGCAGGGCACUGGU | 679 |
| hsa-miR-649_st | 0.093994137 | 22:21388465-21388561 (−) | 22 | AAACCUGUGUUGUUCAAGAGUC | 680 |
| hsa-miR-650_st | −0.050464632 | 22:23165270-23165365 (+) | 21 | AGGAGGCAGCGCUCUCAGGAC | 681 |
| hsa-miR-651_st | 0.119970741 | X:8095006-8095102 (+) | 22 | UUUAGGAUAAGCUUGACUUUUG | 682 |
| hsa-miR-652_st | −0.119768033 | X:109298557-109298654 (+) | 21 | AAUGGCGCCACUAGGGUUGUG | 683 |
| hsa-miR-548d- | 0.076408458 | | 22 | AAAAGUAAUUGUGGUUUUUGCC | 684 |
| 5p_st | | | | | |
| hsa-miR-548d- | 0.430544966 | | 22 | CAAAAACCACAGUUUCUUUUGC | 685 |
| 3p_st | | | | | |
| hsa-miR-661_st | −0.616026446 | 8:145019359-145019447 (−) | 24 | UGCCUGGGUCUCUGGCCUGCGCGU | 686 |
| hsa-miR-662_st | 0.261225179 | 16:820183-820277 (+) | 21 | UCCCACGUUGUGGCCCAGCAG | 687 |
| hsa-miR-663_st | −0.480625269 | 20:26188822-26188914 (−) | 22 | AGGCGGGGCGCCGCGGGACCGC | 688 |
| hsa-miR-449b_st | 0.198658827 | 5:54466474-54466570 (−) | 23 | AGGCAGUGUAUUGUUAGCUGGC | 689 |
| hsa-miR-449b- | −0.560389688 | 5:54466474-54466570 (−) | 22 | CAGCCACAACUACCCUGCCACU | 690 |
| star_st | | | | | |
| hsa-miR-653_st | 0.191868449 | 7:93112072-93112167 (−) | 21 | GUGUUGAAACAAUCUCUACUG | 691 |
| hsa-miR-411_st | 0.136686066 | 14:101489662-101489757 (+) | 21 | UAGUAGACCGUAUAGCGUACG | 692 |
| hsa-miR-411- | 0.120011956 | 14:101489662-101489757 (+) | 22 | UAUGUAACACGGUCCACUAACC | 693 |
| star_st | | | | | |
| hsa-miR-654- | −0.472232433 | 14:101506556-101506636 (+) | 22 | UGGUGGGCCGCAGAACAUGUGC | 694 |
| 5p_st | | | | | |
| hsa-miR-654- | 0.264747585 | 14:101506556-101506636 (+) | 22 | UAUGUCUGCUGACCAUCACCUU | 695 |
| 3p_st | | | | | |
| hsa-miR-655_st | 0.480326216 | 14:101515887-101515983 (+) | 22 | AUAAUACAUGGUUAACCUCUUU | 696 |
| hsa-miR-656_st | −0.264321396 | 14:101533061-101533138 (+) | 21 | AAUAUUAUACAGUCAACCUCU | 697 |
| hsa-miR-549_st | −0.048354407 | 15:81134319-81134414 (−) | 21 | UGACAACUAUGGAUGAGCUCU | 698 |
| hsa-miR-657_st | 0.227310786 | 17:79099076-79099173 (−) | 23 | GGCAGGUUCUCACCCUCUCUAGG | 699 |
| hsa-miR-658_st | −0.529914071 | 22:38240279-38240378 (−) | 25 | GGCGGAGGGAAGUAGGUCCGUUGGU | 700 |
| hsa-miR-659_st | −0.577515457 | 22:38243685-38243781 (−) | 22 | CUUGGUUCAGGGAGGGUCCCCA | 701 |
| hsa-miR-660_st | 0.561018419 | X:49777849-49777945 (+) | 22 | UACCCAUUGCAUAUCGGAGUUG | 702 |
| hsa-miR-421_st | 0.627401792 | X:73438212-73438296 (−) | 23 | AUCAACAGACAUUAAUUGGGCGC | 703 |
| hsa-miR-542- | 0.00378439 | X:133675371-133675467 (−) | 23 | UCGGGGAUCAUCAUGUCACGAGA | 704 |
| 5p_st | | | | | |
| hsa-miR-542- | 0.559020355 | X:133675371-133675467 (−) | 22 | UGUGACAGAUUGAUAACUGAAA | 705 |
| 3p_st | | | | | |
| hsa-miR-758_st | 0.019688671 | 14:101492357-101492444 (+) | 22 | UUUGUGACCUGGUCCACUAACC | 706 |
| hsa-miR-1264_st | 0.452195997 | X:113887130-113887198 (+) | 23 | CAAGUCUUAUUUGAGCACCUGUU | 707 |
| hsa-miR-671- | −0.364430355 | 7:150935507-150935624 (+) | 23 | AGGAAGCCCUGGAGGGGCUGGAG | 708 |
| 5p_st | | | | | |
| hsa-miR-671- | 0.167684298 | 7:150935507-150935624 (+) | 21 | UCCGGUUCUCAGGGCUCCACC | 709 |
| 3p_st | | | | | |
| hsa-miR-668_st | −0.632007016 | 14:101321595-101521660 (+) | 23 | UGUCACUCGGCUCGGCCCACUAC | 710 |
| hsa-miR-767- | 0.125211371 | X:151561893-151562001 (−) | 23 | UGCACCAUGGUUGUCUGAGCAUG | 711 |
| 5p_st | | | | | |
| hsa-miR-767- | 0.241188916 | X:151561893-151562001 (−) | 23 | UCUGCUCAUACCCCAUGGUUUCU | 712 |
| 3p_st | | | | | |
| hsa-miR-1224- | −0.326933758 | 3:183959193-183959277 (+) | 19 | GUGAGGACUCGGGAGGUGG | 713 |
| 5p_st | | | | | |
| hsa-miR-1224- | −0.099987848 | 3:183959193-183959277 (+) | 21 | CCCCACCUCCUCUCUCCUCAG | 714 |
| 3p_st | | | | | |
| hsa-miR-320b_st | −0.353894764 | | 22 | AAAAGCUGGGUUGAGAGGGCAA | 715 |
| hsa-miR-320c_st | −0.148187748 | | 20 | AAAAGCUGGGUUGAGAGGGU | 716 |
| hsa-miR-1296_st | −0.256319335 | 10:65132717-65132808 (−) | 22 | UUAGGGCCCUGGCUCCAUCUCC | 717 |
| hsa-miR-1468_st | −0.153590312 | X:63005882-63005967 (−) | 21 | CUCCGUUUGCCUGUUUCGCUG | 718 |
| hsa-miR-1323_st | −0.496797108 | 19:54175222-54175294 (+) | 22 | UCAAAACUGAGGGGCAUUUUCU | 719 |
| hsa-miR-1271_st | 0.369484935 | 5:175794949-175795034 (+) | 22 | CUUGGCACCUAGCAAGCACUCA | 720 |
| hsa-miR-1301_st | 0.1010758 | 2:25551509-25551590 (−) | 24 | UUGCAGCUGCCUGGGAGUGACUUC | 721 |
| hsa-miR-454- | 0.061800153 | 17:57215119-57215233 (−) | 22 | ACCCUAUCAAUAUUGUCUCUGC | 722 |
| star_st | | | | | |
| hsa-miR-454_st | 1.312831049 | 17:57215119-57215233 (−) | 23 | UAGUGCAAUAUUGCUUAUAGGGU | 723 |
| hsa-miR-1185_st | 9.24573E−05 | | 21 | AGAGGAUACCCUUUGUAUGUU | 724 |
| hsa-miR-449c_st | −0.310930519 | 5:54468090-54468181 (−) | 25 | UAGGCAGUGUAUUGCUAGCGGCUGU | 725 |
| hsa-miR-449c- | −0.471675579 | 5:54468090-54468181 (−) | 23 | UUGCUAGUUGCACUCCUCUCUGU | 726 |
| star_st | | | | | |
| hsa-miR-1283_st | −0.053277143 | | 22 | UCUACAAAGGAAAGCGCUUUCU | 727 |
| hsa-miR-769- | 0.387224935 | 19:46522190-46322307 (+) | 22 | UGAGACCUCUGGGUUCUGAGCU | 728 |
| 5p_st | | | | | |
| hsa-miR-769- | −0.612189973 | 19:46522190-46522307 (+) | 23 | CUGGGAUCUCCGGGGUCUUGGUU | 729 |
| 3p_st | | | | | |
| hsa-miR-766_st | 0.007320627 | X:118780701-118780811 (−) | 22 | ACUCCAGCCCCACAGCCUCAGC | 730 |
| hsa-miR-762_st | −0.446795615 | 16:10905224-30905306 (+) | 22 | GGGGCUGGGGCCGGGGCCGAGC | 731 |
| hsa-miR-802_st | −0.017774103 | 21:37093013-37093106 (+) | 23 | CAGUAACAAAGAUUCAUCCUUGU | 732 |
| hsa-miR-670_st | −0.092740756 | 11:43581206-43581303 (+) | 20 | GUCCCUGAGUGUAUGUGGUG | 733 |
| hsa-miR-1248_st | 0.326000027 | X:113949650-113949761 (+) | 22 | UUCAUUCGGCUGUCCAGAUGUA | 734 |
| hsa-miR-2113_st | 0.14939042 | 6:98472407-98472497 (+) | 21 | AUUUGUGCUUGGCUCUGUCAC | 735 |
| hsa-miR-761_st | −0.335788423 | 1:52302016-52302074 (−) | 22 | GCAGCAGGGUGAAACUGACACA | 736 |
| hsa-miR-764_st | 0.368644739 | X:113873918-113874002 (+) | 22 | GCAGGUGCUCACUUGUCCUCCU | 737 |
| hsa-miR-759_st | 0.388215181 | 13:53384185-53384275 (+) | 22 | GCAGAGUGCAAACAAUUUUGAC | 738 |
| hsa-miR-765_st | −0.265455113 | 1:156905923-156906036 (−) | 21 | UGGAGGAGAAGGAAGGUGAUG | 739 |
| hsa-miR-770- | −0.406433427 | 14:101318727-101318824 (+) | 23 | UCCAGUACCACGUGUCAGGGCCA | 740 |
| 5p_st | | | | | |
| hsa-miR-675_st | −0.465904133 | 11:2017989-2018061 (−) | 23 | UGGUGCGGAGAGGGCCCACAGUG | 741 |
| hsa-miR-675- | −0.733207197 | 11:2017989-2018061 (−) | 20 | CUGUAUGCCCUCACCGCUCA | 742 |
| star_st | | | | | |
| hsa-miR-298_st | −0.342997868 | 20:57393281-57393368 (−) | 23 | AGCAGAAGCAGGGAGGUUCUCCCA | 743 |
| hsa-miR-891a_st | 0.415131889 | X:145109312-145109390 (−) | 22 | UGCAACGAACCUGAGCCACUGA | 744 |
| hsa-miR-300_st | −0.156884271 | 14:101507700-101507782 (+) | 22 | UAUACAAGGGCAGACUCUCUCU | 745 |
| hsa-miR-886- | −0.7111646 | 5:135416177-135416297 (−) | 23 | CGGGUCGGAGUUAGCUCAAGCGG | 746 |
| 5p_st | | | | | |
| hsa-miR-886- | −0.265754631 | 5:135416177-135416297 (−) | 21 | CGCGGGUGCUUACUGACCCUU | 747 |
| 3p_st | | | | | |
| hsa-miR-892a_st | −0.0178816 | X:145078187-145078261 (−) | 21 | CACUGUGUCCUUUCUGCGUAG | 748 |
| hsa-miR-220b_st | −0.183033422 | 19:6495959-6496045 (+) | 21 | CCACCACCGUGUCUGACACUU | 749 |
| hsa-miR-450b- | 0.389809937 | X:133674215-133674292 (−) | 22 | UUUUGCAAUAUGUUCCUGAAUA | 750 |
| 5p_st | | | | | |
| hsa-miR-450b- | 0.049147767 | X:133674215-133674292 (−) | 22 | UUGGGAUCAUUUUGCAUCCAUA | 751 |
| 3p_st | | | | | |
| hsa-miR-874_st | −0.157649315 | 5:136933261-136983338 (−) | 22 | CUGCCCUGGCCCGAGGGACCGA | 752 |
| hsa-miR-890_st | 0.760488224 | X:145075793-145075869 (−) | 21 | UACUUGGAAAGGCAUCAGUUG | 753 |
| hsa-miR-891b_st | 0.743488807 | X:145082571-145082649 (−) | 22 | UGCAACUUACCUGAGUCAUUGA | 754 |
| hsa-miR-220c_st | 0.076507092 | 19:49063529-49063611 (−) | 22 | ACACAGGGCUGUUGUGAAGACU | 755 |
| hsa-miR-888_st | 0.674792334 | X:145076302-145076378 (−) | 21 | UACUCAAAAAGCUGUCAGUCA | 756 |
| hsa-miR-888- | −0.055511439 | X:145076302-145076378 (−) | 22 | GACUGACACCUCUUUGGGUGAA | 757 |
| star_st | | | | | |
| hsa-miR-892b_st | 0.58203853 | X:145078716-145078792 (−) | 22 | CACUGGCUCCUUUCUGGGUAGA | 758 |
| hsa-miR-541- | −0.270351489 | 14:101530832-101530915 (+) | 25 | AAAGGAUUCUGCUGUCGGUCCCACU | 759 |
| star_st | | | | | |
| hsa-miR-541_st | −0.426868452 | 14:101530832-101530915 (+) | 22 | UGGUGGGCACAGAAUCUGGACU | 760 |
| hsa-miR-889_st | 0.168340573 | 14:101514238-101514316 (+) | 21 | UUAAUAUCGGACAACCAUUGU | 761 |
| hsa-miR-875- | −0.464819734 | 8:100549014-100549089 (−) | 22 | UAUACCUCAGUUUUAUCAGGUG | 762 |
| 5p_st | | | | | |
| hsa-miR-875- | 0.265824374 | 8:100549014-100549089 (−) | 21 | CCUGGAAACACUGAGGUUGUG | 763 |
| 3p_st | | | | | |
| hsa-miR-876- | −0.212025682 | 9:28863624-28863704 (−) | 22 | UGGAUUUCUUUGUGAAUCACCA | 764 |
| 5p_st | | | | | |
| hsa-miR-876- | 0.101773622 | 9:28863624-28863704 (−) | 22 | UGGUGGUUUACAAAGUAAUUCA | 765 |
| 3p_st | | | | | |
| hsa-miR-708_st | −0.148807187 | 11:79113066-79113153 (−) | 23 | AAGGAGCUUACAAUCUAGCUGGG | 766 |
| hsa-miR-708- | −0.25648189 | 11:79113066-79113153 (−) | 22 | CAACUAGACUGUGAGCUUCUAG | 767 |
| star_st | | | | | |
| hsa-miR-147b_st | −0.12357752 | 15:45725248-45725327 (+) | 22 | GUGUGCGGAAAUGCUUCUGCUA | 768 |
| hsa-miR-190b_st | −0.653593416 | 1:154166141-154166219 (−) | 21 | UGAUAUGUUUGAUAUUGGGUU | 769 |
| hsa-miR-744_st | −0.439822595 | 17:11985216-11985313 (+) | 23 | UGCGGGGCUAGGGCUAACAGCA | 770 |
| hsa-miR-744- | 0.378794923 | 17:11985216-11985313 (+) | 22 | CUGUUGCCACUAACCUCAACCU | 771 |
| star_st | | | | | |
| hsa-miR-885- | −0.916203074 | 3:10436173-10436246 (−) | 22 | UCCAUUACACUACCCUGCCUCU | 772 |
| 5p_st | | | | | |
| hsa-miR-885- | −0.75194678 | 3:10436173-10436246 (−) | 22 | AGGCAGCGGGGUGUAGUGGAUA | 773 |
| 3p_st | | | | | |
| hsa-miR-877_st | −0.324294781 | 6:30552109-30552194 (+) | 20 | GUAGAGGAGAUGGCGCAGGG | 774 |
| hsa-miR-877- | −0.400447381 | 6:30552109-30552194 (+) | 21 | UCCUCUUCUCCCUCCUCCCAG | 775 |
| star_st | | | | | |
| hsa-miR-887_st | −0.262112859 | 5:15935291-15935369 (+) | 22 | GUGAACGGGCGCCAUCCCGAGG | 776 |
| hsa-miR-665_st | −0.176767659 | 14:101341370-101341441 (+) | 20 | ACCAGGAGGCUGAGGCCCCU | 777 |
| hsa-miR-873_st | −0.070415832 | 9:28888877-28888953 (−) | 21 | GCAGGAACUUGUGAGUCUCCU | 778 |
| hsa-miR-543_st | 0.386383186 | 14:101498324-101498401 (+) | 22 | AAACAUUCGCGGUGCACUUCUU | 779 |
| hsa-miR-374b_st | 1.079803793 | X:73438382-73438453 (−) | 22 | AUAUAAUACAACCUGCUAAGUG | 780 |
| hsa-miR-374b- | 0.489481199 | X:73438382-73438453 (−) | 22 | CUUAGCAGGUUGUAUUAUCAUU | 781 |
| star_st | | | | | |
| hsa-miR-760_st | −0.674938847 | 1:94312388-94312467 (+) | 20 | CGGCUCUGGGUCUGUGGGGA | 782 |
| hsa-miR-301b_st | 0.794315923 | 22:22007270-22007347 (+) | 23 | CAGUGCAAUGAUAUUGUCAAAGC | 783 |
| hsa-miR-216b_st | 0.511660042 | 2:56227849-56227930 (−) | 22 | AAAUCUCUGCAGGCAAAUGUGA | 784 |
| hsa-miR-208b_st | 0.302538439 | 14:23887196-23887272 (−) | 22 | AUAAGACGAACAAAAGGUUUGU | 785 |
| hsa-miR-920_st | −0.576255005 | 12:24365355-24365429 (+) | 20 | GGGGAGCUGUGGAAGCAGUA | 786 |
| hsa-miR-921_st | −0.286412503 | 1:166123980-166124035 (−) | 25 | CUAGUGAGGGACAGAACCAGGAUUC | 787 |
| hsa-miR-922_st | −0.138833401 | 3:197401567-197401447 (−) | 23 | GCAGCAGAGAAUAGGACUACGUC | 788 |
| hsa-miR-924_st | 0.582023843 | 18:37202087-37202139 (−) | 20 | AGAGUCUUGUGAUGUCUUGC | 789 |
| hsa-miR-509-3- | −0.317242543 | X:146341170-146341244 (−) | 22 | UACUGCAGACGUGGCAAUCAUG | 790 |
| 5p_st | | | | | |
| hsa-miR-933_st | 0.050795443 | 2:176032361-176032437 (−) | 22 | UGUGCGCAGGGAGACCUCUCCC | 791 |
| hsa-miR-934_st | −0.008209299 | X:135633037-135633119 (+) | 22 | UGUCUACUACUGGAGACACUGG | 792 |
| hsa-miR-935_st | −0.743708446 | 19:54485561-54485651 (+) | 23 | CCAGUUACCGCUUCCGCUACCGC | 793 |
| hsa-miR-936_st | −0.279060211 | 10:105807847-105807944 (−) | 22 | ACAGUAGAGGGAGGAAUCGCAG | 794 |
| hsa-miR-937_st | 0.040672517 | 8:144895127-144895212 (−) | 22 | AUCCGCGCUCUGACUCUCUGCC | 795 |
| hsa-miR-938_st | −0.20978976 | 10:29891193-29891275 (−) | 22 | UGCCCUUAAAGGUGAACCCAGU | 796 |
| hsa-miR-939_st | −0.579938374 | 8:145619364-145619445 (−) | 24 | UGGGGAGCUGAGGCUCUGGGGGUG | 797 |
| hsa-miR-940_st | −0.272587138 | 16:2321748-2321841 (+) | 21 | AAGGCAGGGCCCCCGCUCCCC | 798 |
| hsa-miR-941_st | −0.068050295 | | 23 | CACCCGGCUGUGUGCACAUGUGC | 799 |
| hsa-miR-942_st | 0.366729056 | 1:117637265-117637350 (+) | 22 | UCUUCUCUGUUUUGGCCAUGUG | 800 |
| hsa-miR-943_st | −0.806599985 | 4:1988111-1988204 (−) | 21 | CUGACUGUUGCCGUCCUCCAG | 801 |
| hsa-miR-944_st | 0.074666201 | 3:189547711-189547798 (+) | 22 | AAAUUAUUGUACAUCGGAUGAG | 802 |
| hsa-miR-297_st | 0.348144584 | 4:111781738-111781803 (−) | 21 | AUGUAUGUGUGCAUGUGCAUG | 803 |
| hsa-miR-1178_st | −0.118722857 | 12:120151439-120151529 (−) | 21 | UUGCUCACUGUUCUUCCCUAG | 804 |
| hsa-miR-1179_st | 0.113052871 | 15:89151338-89151428 (+) | 21 | AAGCAUUCUUUCAUUGGUUGG | 805 |
| hsa-miR-1180_st | −0.488976108 | 17:19247819-19247887 (−) | 22 | UUUCCGGCUCGCGUGGGUGUGU | 806 |
| hsa-miR-1181_st | −0.553946496 | 19:10514134-10514214 (−) | 21 | CCGUCGCCGCCACCCGAGCCG | 807 |
| hsa-miR-1182_st | −0.457543047 | 1:231155574-231155670 (−) | 23 | GAGGGUCUUGGGAGGGAUGUGAC | 808 |
| hsa-miR-1183_st | −0.351318092 | 7:21510676-21510764 (+) | 27 | CACUGUAGGUGAUGGUGAGAGUGG | 809 |
| | | | | GCA | |
| hsa-miR-1184_st | −0.227475628 | | 23 | CCUGCAGCGACUUGAUGGCUUCC | 810 |
| hsa-miR-1225- | −0.38719767 | 16:2140196-2140285 (−) | 22 | GUGGGUACGGCCCAGUGGGGGG | 811 |
| 5p_st | | | | | |
| hsa-miR-1225- | −0.607399326 | 16:2140196-2140285 (−) | 22 | UGAGCCCCUGUGCCGCCCCCAG | 812 |
| 3p_st | | | | | |
| hsa-miR-1226- | −0.396569371 | 3:47891045-47891119 (+) | 26 | GUGAGGGCAUGCAGGCCUGGAUGG | 813 |
| star_st | | | | GG | |
| hsa-miR-1226_st | −0.164177373 | 3:47891045-47891119 (+) | 22 | UCACCAGCCCUGUGUUCCCUAG | 814 |
| hsa-miR-1227_st | −0.392136136 | 19:2234061-2234148 (−) | 20 | CGUGCCACCCUUUUCCCCAG | 815 |
| hsa-miR-1228- | −0.456494317 | 12:57588287-57588359 (+) | 21 | GUGGGCGGGGGCAGGUGUGUG | 816 |
| star_st | | | | | |
| hsa-miR-1228_st | −0.429305902 | 12:57588287-57588359 (+) | 20 | UCACACCUGCCUCGCCCCCC | 817 |
| hsa-miR-1229_st | −0.440646346 | 5:179225278-179225346 (−) | 23 | CUCUCACCACUGCCCUCCCACAG | 818 |
| hsa-miR-1231_st | −0.698665174 | 1:201777739-201777830 (+) | 20 | GUGUCUGGGCGGACAGCUGC | 819 |
| hsa-miR-1233_st | −0.327747499 | | 20 | UGAGCCCUGUCCUCCCGCAG | 820 |
| hsa-miR-1234_st | −0.274407678 | 8:145625476-145625559 (−) | 22 | UCGGCCUGACCACCCACCCCAC | 821 |
| hsa-miR-1236_st | −0.195255274 | 6:31924616-31924717 (−) | 22 | CCUCUUCCCCUUGUCUCUCCAG | 822 |
| hsa-miR-1237_st | −0.547769245 | 11:64136074-64136175 (+) | 21 | UCCUUCUCCUCCGUCCCCCAG | 823 |
| hsa-miR-1238_st | −0.255447436 | 19:10662798-10662880 (+) | 20 | CUUCCUCGUCUGUCUGCCCC | 824 |
| hsa-miR-1200_st | −0.358476635 | 7:36958962-36959037 (−) | 22 | CUCCUGAGCCAUUCUGAGCCUC | 825 |
| hsa-miR-1201_st | 0.77001394 | 14:20794606-20794690 (−) | 24 | AGCCUGAUUAAACACAUGCUCUGA | 826 |
| hsa-miR-1202_st | −0.539600384 | 6:156267931-156268013 (+) | 21 | GUGCCAGCUGCAGUGGGGGAG | 827 |
| hsa-miR-1203_st | −0.098483453 | 17:46233789-46233873 (−) | 21 | CCCGGAGCCAGGAUGCAGCUC | 828 |
| hsa-miR-663b_st | −0.07854292 | 2:133014539-133014653 (−) | 22 | GGUGGCCCGGCCGUGCCUGAGG | 829 |
| hsa-miR-1204_st | −0.38023458 | 8:128808208-128808274 (+) | 21 | UCGUGGCCUGGUCUCCAUUAU | 830 |
| hsa-miR-1205_st | 0.119236107 | 8:128972879-128972941 (+) | 20 | UCUGCAGGGUUUGCUUUGAG | 831 |
| hsa-miR-1206_st | 0.61109876 | 8:129021144-129021202 (+) | 21 | UGUUCAUGUAGAUGUUUAAGC | 832 |
| hsa-miR-1207- | −0.413891024 | 8:129061398-129061484 (+) | 21 | UGGCAGGGAGGCUGGGAGGGG | 833 |
| 5p_st | | | | | |
| hsa-miR-1207- | −0.590440262 | 8:129061398-129061484 (+) | 18 | UCAGCUGGCCCUCAUUUC | 834 |
| 3p_st | | | | | |
| hsa-miR-1208_st | −0.697774512 | 8:129162362-129162434 (+) | 20 | UCACUGUUCAGACAGGCGGA | 835 |
| hsa-miR-548e_st | 0.342367468 | 10:112748684-112748771 (+) | 22 | AAAAACUGAGACUACUUUUGCA | 836 |
| hsa-miR-548j_st | −0.378682535 | 22:26951178-26951289 (−) | 22 | AAAAGUAAUUGCGGUCUUUGGU | 837 |
| hsa-miR-1285_st | −0.112973054 | | 22 | UCUGGGCAACAAAGUGAGACCU | 838 |
| hsa-miR-1286_st | −0.329534325 | 22:20236657-20236734 (−) | 21 | UGCAGGACCAAGAUGAGCCCU | 839 |
| hsa-miR-1287_st | 0.155123326 | 10:100154975-100155064 (−) | 22 | UGCUGGAUCAGUGGUUCGAGUC | 840 |
| hsa-miR-1289_st | 0.109012835 | | 23 | UGGAGUCCAGGAAUCUGCAUUUU | 841 |
| hsa-miR-1290_st | −0.35417404 | 1:19223565-19223642 (−) | 19 | UGGAUUUUUGGAUCAGGGA | 842 |
| hsa-miR-1291_st | −0.145049331 | 12:49048227-49048313 (−) | 24 | UGGCCCUGACUGAAGACCAGCAGU | 843 |
| hsa-miR-548k_st | 0.508991181 | 11:70130061-70130176 (+) | 22 | AAAAGUACUUGCGGAUUUUGCU | 844 |
| hsa-miR-1293_st | −0.341052122 | 12:50627925-50627995 (−) | 22 | UGGGUGGUCUGGAGAUUUGUGC | 845 |
| hsa-miR-1294_st | −0.185000925 | 5:153726666-153726807 (+) | 22 | UGUGAGGUUGGCAUUGUUGUCU | 846 |
| hsa-miR-1295_st | −0.087279455 | 1:171070869-171070947 (−) | 21 | UUAGGCCGCAGAUCUGGGUGA | 847 |
| hsa-miR-1297_st | 0.579269966 | 13:54886107-54886183 (−) | 17 | UUCAAGUAAUUCAGGUG | 848 |
| hsa-miR-1299_st | −0.328354408 | 9:69002239-69002321 (−) | 22 | UUCUGGAAUUCUGUGUGAGGGA | 849 |
| hsa-miR-5481_st | −0.098584184 | 11:94199661-94199746 (−) | 22 | AAAAGUAUUUGCGGGUUUUGUC | 850 |
| hsa-miR-1302_st | −0.052798803 | | 21 | UUGGGACAUACUUAUGCUAAA | 851 |
| hsa-miR-1303_st | −0.347023877 | 5:154065336-154065421 (+) | 22 | UUUAGAGACGGGGUCUUGCUCU | 852 |
| hsa-miR-1304_st | 0.348603582 | 11:93466840-93466930 (−) | 22 | UUUGAGGCUACAGUGAGAUGUG | 853 |
| hsa-miR-1305_st | 0.076731378 | 4:183090446-183090531 (+) | 22 | UUUUCAACUCUAAUGGGAGAGA | 854 |
| hsa-miR-1243_st | −0.207192555 | 4:114028019-114028111 (+) | 22 | AACUGGAUCAAUUAUAGGAGUG | 855 |
| hsa-miR-548f_st | −0.1472914 | | 19 | AAAAACUGUAAUUACUUUU | 856 |
| hsa-miR-1244_st | 0.517268072 | | 26 | AAGUAGUUGGUUUGUAUGAGAUGG | 857 |
| | | | | UU | |
| hsa-miR-1245_st | 0.003252269 | 2:189842818-189842887 (+) | 21 | AAGUGAUCUAAAGGCCUACAU | 858 |
| hsa-miR-1246_st | −0.159547883 | 2:177465708-177465780 (−) | 19 | AAUGGAUUUUUGGAGCAGG | 859 |
| hsa-miR-1247_st | −0.327805004 | 14:102026624-l02026759 (−) | 22 | ACCCGUCCCGUUCGUCCCCGGA | 860 |
| hsa-miR-1248_st | 0.360334937 | 3:186504461-186504566 (+) | 27 | ACCUUCUUGUAUAAGCACUGUGCU | 861 |
| | | | | AAA | |
| hsa-miR-1249_st | 0.043587535 | 22:45596835-45596900 (−) | 22 | ACGCCCUUCCCCCCCUUCUUCA | 862 |
| hsa-miR-1250_st | −0.336528856 | 17:79106996-79107108 (−) | 21 | ACGGUGCUGGAUGUGGCCUUU | 863 |
| hsa-miR-1251_st | 0.512971088 | 12:97885687-97885756 (+) | 21 | ACUCUAGCUGCCAAAGGCGCU | 864 |
| hsa-miR-1253_st | −0.15538055 | 17:2651372-2651476 (−) | 21 | AGAGAAGAAGAUCAGCCUGCA | 865 |
| hsa-miR-1254_st | −0.233801498 | 10:70519075-70519171 (+) | 24 | AGCCUGGAAGCUGGAGCCUGCAGU | 866 |
| hsa-miR-1255a_st | 0.187915657 | 4:102251459-102251571 (−) | 23 | AGGAUGAGCAAAGAAAGUAGAUU | 867 |
| hsa-miR-1256_st | 0.33571115 | 1:21314807-21314925 (−) | 22 | AGGCAUUGACUUCUCACUAGCU | 868 |
| hsa-miR-1257_st | 0.124103955 | 20:60528602-60528718 (−) | 21 | AGUGAAUGAUGGGUUCUGACC | 869 |
| hsa-miR-1258_st | 0.043788417 | 2:180725563-180725635 (−) | 21 | AGUUAGGAUUAGGUCGUGGAA | 870 |
| hsa-miR-1259_st | 0.34061951 | 20:47896847-47896957 (+) | 21 | AUAUAUGAUGACUUAGCUUUU | 871 |
| hsa-miR-1260_st | 0.016061699 | 14:77732561-77732633 (+) | 18 | AUCCCACCUCUGCCACCA | 872 |
| hsa-miR-548g_st | 0.211530862 | 4:148265781-148265869 (−) | 22 | AAAACUGUAAUUACUUUUGUAC | 873 |
| hsa-miR-1261_st | −0.112273789 | 11:90602289-90602370 (−) | 19 | AUGGAUAAGGCUUUGGCUU | 874 |
| hsa-miR-1262_st | −0.200157382 | 1:68649201-68649293 (−) | 22 | AUGGGUGAAUUUGUAGAAGGAU | 875 |
| hsa-miR-1263_st | −0.351098595 | 3:163889259-163889344 (−) | 22 | AUGGUACCCUGGCAUACUGAGU | 876 |
| hsa-miR-548n_st | 0.042829555 | 7:34980372-34980446 (−) | 22 | CAAAAGUAAUUGUGGAUUUUGU | 877 |
| hsa-miR-548m_st | 0.297403266 | X:94318140-94318225 (−) | 21 | CAAAGGUAUUUGUGGUUUUUG | 878 |
| hsa-miR-1265_st | −0.169136532 | 10:14478575-14478660 (+) | 22 | CAGGAUGUGGUCAAGUGUUGUU | 879 |
| hsa-miR-548o_st | −0.431360601 | 7:102046189-102046302 (−) | 22 | CCAAAACUGCAGUUACUUUUGC | 880 |
| hsa-miR-1266_St | −0.491370046 | 15:52569314-52569397 (−) | 23 | CCUCAGGGCUGUAGAACAGGGCU | 881 |
| hsa-miR-1267_st | −0.375000166 | 13:108183519-108183596 (−) | 21 | CCUGUUGAAGUGUAAUCCCCA | 882 |
| hsa-miR-1268_st | −0.475127329 | 15:22513229-22513280 (−) | 18 | CGGGCGUGGUGGUGGGGG | 883 |
| hsa-miR-1269_st | −0.064540701 | 4:67142542-67142646 (+) | 22 | CUGGACUGAGCCGUGCUACUGG | 884 |
| hsa-miR-1270_st | −0.125725591 | | 23 | CUGGAGAUAUGGAAGAGCUGUGU | 885 |
| hsa-miR-1272_st | −0.024170509 | 15:65054586-65054714 (−) | 26 | GAUGAUGAUGGCAGCAAAUUCUGA | 886 |
| | | | | AA | |
| hsa-miR-1273_st | −0.092136851 | 8:101036210-101036312 (−) | 25 | GGGCGACAAAGCAAGACUCUUUCUU | 887 |
| hsa-miR- | 1.087481971 | 5:41475734-41475804 (+) | 18 | GUCCCUGUUCAGGCGCCA | 888 |
| 1274n_st | | | | | |
| hsa-miR-548h_st | 0.36654465 | | 22 | AAAAGUAAUCGCGGUUUUUGUC | 889 |
| hsa-miR-1275_st | −0.378876303 | 6:33967749-33967828 (−) | 17 | GUGGGGGAGAGGCUGUC | 890 |
| hsa-miR-1276_st | −0.208238828 | 15:86313727-86313809 (−) | 20 | UAAAGAGCCCUGUGGAGACA | 891 |
| hsa-miR-302e_st | −0.354429514 | 11:7255997-7256068 (+) | 17 | UAAGUGCUUCCAUGCUU | 892 |
| hsa-miR-302f_st | 0.403378813 | 18:27878876-27878926 (+) | 17 | UAAUUGCUUCCAUGUUU | 893 |
| hsa-miR-1277_st | −0.046188492 | X:117520357-117520434 (+) | 22 | UACGUAGAUAUAUAUGUAUUUU | 894 |
| hsa-miR-548p_st | 0.147242156 | 5:100152186-100152269 (−) | 22 | UAGCAAAAACUGCAGUUACUUU | 895 |
| hsa-miR-548i_st | 0.085843127 | | 22 | AAAAGUAAUUGCGGAUUUUGCC | 896 |
| hsa-miR-1278_st | 0.140507622 | 1:193105633-193105713 (+) | 22 | UAGUACUGUGCAUAUCAUCUAU | 897 |
| hsa-miR-1279_st | 0.497329076 | 12:69666937-69666998 (−) | 17 | UCAUAUUGCUUCUUUCU | 898 |
| hsa-miR-1274b_st | 0.650599882 | 19:58024375-58024441 (−) | 17 | UCCCUGUUCGGGCGCCA | 899 |
| hsa-miR-1281_st | −0.534051953 | 22:41488517-41488570 (+) | 17 | UCGCCUCCUCCUCUCCC | 900 |
| hsa-miR-1282_st | 0.282040051 | 15:44085857-44085957 (−) | 20 | UCGUUUGCCUUUUUCUGCUU | 901 |
| hsa-miR-1284_st | −0.066820239 | 3:71591121-71591240 (−) | 22 | UCUAUACAGACCCUGGCUUUUC | 902 |
| hsa-miR-1288_st | 0.252079407 | 17:16185328-16185402 (+) | 21 | UGGACUGCCCUGAUCUGGAGA | 903 |
| hsa-miR-1292_st | −0.42886343 | 20:2633423-2633488 (+) | 25 | UGGGAACGGGUUCCGGCAGACGCUG | 904 |
| hsa-miR-1252_st | 0.250812674 | 12:79813037-79813101 (+) | 22 | AGAAGGAAAUUGAAUUCAUUUA | 905 |
| hsa-miR-1255b_st | 0.349514198 | | 22 | CGGAUGAGCAAAGAAAGUGGUU | 906 |
| hsa-miR-1280_st | −0.049084714 | 3:128081008-128081101 (+) | 17 | UCCCACCGCUGCCACCC | 907 |
| hsa-miR-1308_st | 0.168161553 | X:22080259-22080312 (−) | 18 | GCAUGGGUGGUUCAGUGG | 908 |
| hsa-miR-664- | −0.356675666 | 1:220373880-220373961 (−) | 24 | ACUGGCUAGGGAAAAUGAUUGGAU | 909 |
| star_st | | | | | |
| hsa-miR-664_st | −0.205960725 | 1:220373880-220373961 (−) | 23 | UAUUCAUUUAUCCCCAGCCUACA | 910 |
| hsa-miR-1306_st | −0.288724376 | 22:20073581-20073665 (+) | 18 | ACGUUGGCUCUGGUGGUG | 911 |
| hsa-miR-1307_st | −0.60493163 | 10:105154010-105154158 (−) | 22 | ACUCGGCGUGGCGUCGGUCGUG | 912 |
| hsa-miR-513b_st | −0.117368926 | X:146280562-146280645 (−) | 22 | UUCACAAGGAGGUGUCAUUUAU | 913 |
| hsa-miR-513c_st | −0.391776892 | X:146271222-146271305 (−) | 22 | UUCUCAAGGAGGUGUCGUUUAU | 914 |
| hsa-miR-1321_st | −0.430473503 | X:85090785-85090863 (+) | 18 | CAGGGAGGUGAAUGUGAU | 915 |
| hsa-miR-1322_st | −0.112231232 | 8:10682883-10682953 (−) | 19 | GAUGAUGCUGCUGAUGCUG | 916 |
| hsa-miR-720_st | 0.023319864 | 3:164059129-164059238 (+) | 17 | UCUCGCUGGGGCCUCCA | 917 |
| hsa-miR-1197_st | 0.090310683 | 14:101491901-101491988 (+) | 21 | UAGGACACAUGGUCUACUUCU | 918 |
| hsa-miR-1324_st | 0.4334564 | 3:75679914-75680009 (+) | 24 | CCAGACAGAAUUCUAUGCACUUUC | 919 |
| hsa-miR-1469_st | −0.470797377 | 15:96876490-96876536 (+) | 22 | CUCGGCGCGGGGCGCGGGCUCC | 920 |
| hsa-miR-1470_st | −0.596380055 | 19:15560359-15560419 (+) | 21 | GCCCUCCGCCCGUGCACCCCG | 921 |
| hsa-miR-1471_st | −0.38161882 | 2:232756952-232757008 (−) | 22 | GCCCGCGUGUGGAGCCAGGUGU | 922 |
| hsa-miR-1537_st | 0.23022374 | 1:236016300-236016360 (−) | 22 | AAAACCGUCUAGUUACAGUUGU | 923 |
| hsa-miR-1538_st | −0.165515652 | 16:69399711-69599771 (−) | 23 | CGGCCCGGGCUGCUGCUGUUCCU | 924 |
| hsa-miR-1519_st | 0.003073749 | 18:47013743-47013792 (+) | 21 | UCCUGCGCGUCCCAGAUGCCC | 925 |
| hsa-miR-103- | −0.241290715 | | 23 | UCAUAGCCCUGUACAAUGCUGCU | 926 |
| as_st | | | | | |
| hsa-miR-320d_st | 0.038797836 | | 19 | AAAAGCUGGGUUGAGAGGA | 927 |
| hsa-miR-1825_st | −0.383306722 | 20:30825598-30825650 (+) | 18 | UCCAGUGCCCUCCUCUCC | 928 |
| hsa-miR-1826_st | −0.274083166 | 16:33965508-33965592 (+) | 27 | AUUGAUCAUCGACACUUCGAACGC | 929 |
| | | | | AAU | |
| hsa-miR-1827_st | 1.177531932 | 12:100583662-100583727 (+) | 18 | UGAGGCAGUAGAUUGAAU | 930 |
| hsa-miR-1908_st | −0.425800688 | 11:61582633-61582712 (−) | 21 | CGGCGGGGGACGGCGAUUGGUC | 931 |
| hsa-miR-1909- | −0.653633397 | 19:1816158-1816237 (−) | 21 | UGAGUGCCGGUGCCUGCCCUG | 932 |
| star_st | | | | | |
| hsa-miR-1909_st | −0.507939701 | 19:1816158-1816237 (−) | 22 | CGCAGGGGCCGGGUGCUCACCG | 933 |
| hsa-miR-1910_st | −1.031190911 | 16:85775227-85775306 (−) | 21 | CCAGUCCUGUGCCUGCCGCCU | 934 |
| hsa-miR-1911_st | 0.078126657 | X:113997744-113997823 (+) | 23 | UGAGUACCGCCAUGUCUGUUGGG | 935 |
| hsa-miR-1911- | 0.100849161 | X:113997744-113997823 (+) | 20 | CACCAGGCAUUGUGGUCUCC | 936 |
| star_st | | | | | |
| hsa-miR-1912_st | 0.111721744 | X:113886019-113886098 (+) | 22 | UACCCAGAGCAUGCAGUGUGAA | 937 |
| hsa-miR-1913_st | −0.392395342 | 6:166922842-166922921 (−) | 22 | UCUGCCCCCUCCGCUGCUGCCA | 938 |
| hsa-miR-1914_st | −0.30704752 | 20:62572818-62572897 (−) | 22 | CCCUGUGCCCGGCCCACUUCUG | 939 |
| hsa-miR-1914- | −0.480039303 | 20:62572818-62572897 (−) | 22 | GGAGGGGUCCCGCACUGGGAGG | 940 |
| star_st | | | | | |
| hsa-miR-1915- | −0.509178313 | 10:21785491-21785570 (−) | 22 | ACCUUGCCUUGCUGCCCGGGCC | 941 |
| star_st | | | | | |
| hsa-miR-1915_st | −0.437756775 | 10:21785491-21785570 (−) | 20 | CCCCAGGGCGACGCGGCGGG | 942 |
| hsa-miR-1972_st | −0.200757156 | | 22 | UCAGGCCAGGCACAGUGGCUCA | 943 |
| hsa-miR-1973_st | 0.248567729 | 4:117220881-117220924 (+) | 19 | ACCGUGCAAAGGUAGCAUA | 944 |
| hsa-miR-1975_st | −0.221811549 | 7:148638580-198638654 (+) | 25 | CCCCCACAACCGCGCUUGACUAGCU | 945 |
| hsa-miR-1976_st | −0.631313814 | 1:26881033-26881084 (+) | 20 | CCUCCUGCCCUCCUUGCUGU | 946 |
| hsa-miR-1979_st | −0.254099361 | 4:166321814-166321889 (−) | 22 | CUCCCACUGCUUCACUUGACUA | 947 |
| hsa-miR-2052_st | 0.617914743 | 8:75617928-75617982 (+) | 20 | UGUUUUGAUAACAGUAAUGU | 948 |
| hsa-miR-2053_st | 0.205633242 | 8:113655722-113655812 (+) | 23 | GUGUAAUAUAAACCUCUAUUUAC | 949 |
| hsa-miR-2054_st | 0.31658574 | 4:126428414-126418462 (+) | 22 | CUGUAAUAUAAAUUUAAUUUAUU | 950 |
| hsa-miR-2110_st | −0.438297349 | 10:115933864-115933938 (−) | 22 | UUGGGGAAACGGCCGCUGAGUG | 951 |
| hsa-miR-2114_st | 0.377747857 | X:149396239-149396318 (+) | 22 | UAGUCCCUUCCUUGAAGCGGUC | 952 |
| hsa-miR-2114- | 0.197327215 | X:149396239-149396318 (+) | 21 | CGAGCCUCAAGCAAGGGACUU | 953 |
| star_st | | | | | |
| hsa-miR-2115_st | 0.356015555 | 3:48357850-48357949 (−) | 22 | AGCUUCCAUGACUCCUGAUGGA | 954 |
| hsa-miR-2115- | −0.350063425 | 3:48357850-48357949 (−) | 22 | CAUCAGAAUUCAUGGAGGCUAG | 955 |
| star_st | | | | | |
| hsa-miR-2116_st | −0.081615979 | 15:59463382-59463461 (−) | 21 | GGUUCUUAGCAUAGGAGGUCU | 956 |
| hsa-miR-2116- | 0.257021767 | 15:59463382-59463461 (−) | 21 | CCUCCCAUGCCAAGAACUCCC | 957 |
| star_st | | | | | |
| hsa-miR-2117_st | −0.170797696 | 17:41522174-41522253 (+) | 21 | UGUUCUCUUUGCCAAGGACAG | 958 |
| hsa-miR-548q_st | −0.077430639 | 10:12767253-12767352 (−) | 22 | GCUGGUGCAAAAGUAAUGGCGG | 959 |
| hsa-miR-2276_st | −0.435089246 | 13:24736555-24736643 (+) | 22 | UCUGCAAGUGUCAGAGGCGAGG | 960 |
| hsa-miR-2277_st | −0.600588355 | 5:92956402-92956494 (−) | 21 | UGACAGCGCCCUGCCUGGCUC | 961 |
| hsa-miR-2278_st | −0.296445646 | 9:97572244-97572339 (+) | 22 | GAGAGCAGUGUGUGUUGCCUGG | 962 |
| hsa-miR-711_st | −0.539452913 | 3:48616335-48616410 (−) | 22 | GGGACCCAGGGAGAGACGUAAG | 963 |
| hsa-miR-718_st | −0.278782479 | X:153285371-153285440 (−) | 21 | CUUCCGCCCCGCCGGGCGUCG | 964 |
| hsa-miR-2861_st | −0.420131134 | 9:130548197-130548286 (+) | 19 | GGGGCCUGGCGGUGGGCGG | 965 |
| hsa-miR-2909_st | 0.215130051 | 17:35391042-35391110 (+) | 21 | GUUAGGGCCAACAUCUCUUGG | 966 |
| hsa-miR-3115_st | 0.158204368 | 1:23370798-23370865 (+) | 20 | AUAUGGGUUUACUAGUUGGU | 967 |
| hsa-miR-3116_st | 0.148122118 | | 22 | UGCCUGGAACAUAGUAGGGACU | 968 |
| hsa-miR-3117_st | −0.025995803 | 1:67094123-61094200 (+) | 21 | AUAGGACUCAUAUAGUGCCAG | 969 |
| hsa-miR-3118_st | 0.389328538 | | 23 | UGUGACUGCAUUAUGAAAAUUCU | 970 |
| hsa-miR-3119_st | 0.084756445 | | 20 | UGGCUUUUAACUUUGAUGGC | 971 |
| hsa-miR-3120_st | −0.350893422 | 1:172107948-172108028 (+) | 21 | CACAGCAAGUGUAGACAGGCA | 972 |
| hsa-miR-3121_st | −0.140416111 | 1:180407449-180407525 (−) | 22 | UAAAUAGAGUAGGCAAAGGACA | 973 |
| hsa-miR-3122_st | −0.592901474 | 1:212250955-212251027 (+) | 22 | GUUGGGACAAGAGGACGGUCUU | 974 |
| hsa-miR-3123_st | −0.99978109 | 1:241295572-241295646 (+) | 17 | CAGAGAAUUGUUUAAUC | 975 |
| hsa-miR-3124_st | −0.555102495 | 1:249120576-249120642 (+) | 21 | UUCGCGGGCGAAGGCAAAGUC | 976 |
| hsa-miR-548s_st | 0.223477901 | 2:11907570-11907651 (+) | 23 | AUGGCCAAAACUGCAGUUAUUUU | 977 |
| hsa-miR-3125_st | −0.057958131 | 2:12877493-12877570 (+) | 20 | UAGAGGAAGCUGUGGAGAGA | 978 |
| hsa-miR-3126- | −0.326534467 | 2:69330814-69330887 (+) | 22 | UGAGGGACAGAUGCCAGAAGCA | 979 |
| 5p_st | | | | | |
| hsa-miR-3126- | −0.03094953 | 2:69330814-69330887 (+) | 22 | CAUCUGGCAUCCGUCACACAGA | 980 |
| 3p_st | | | | | |
| hsa-miR-3127_st | −0.256018018 | 2:97464015-97464090 (+) | 23 | AUCAGGGCUUGUGGAAUGGGAAG | 981 |
| hsa-miR-3128_st | −0.157486124 | 2:178120673-178120738 (−) | 23 | UCUGGCAAGUAAAAAACUCUCAU | 982 |
| hsa-MiR-3129_st | −0.209599553 | 2:189997762-189997837 (−) | 22 | GCAGUAGUGUAGAGAUUGGUUU | 983 |
| hsa-miR-3130- | −0.252446104 | | 21 | UACCCAGUCUCCGGUGCAGCC | 984 |
| 5p_st | | | | | |
| hsa-miR-3130- | −0.538355669 | | 21 | GCUGCACCGGAGACUGGGUAA | 985 |
| 3p_st | | | | | |
| hsa-miR-3131_st | −0.502950205 | 2:219923410-219923472 (−) | 23 | UCGAGGACUGGUGGAAGGGCCUU | 986 |
| hsa-miR-3132_st | −0.184592247 | 2:220413795-220413869 (−) | 24 | UGGGUAGAGAAGGAGCUCAGAGGA | 987 |
| hsa-miR-3133_st | 0.324759964 | 2:242417320-242417397 (+) | 22 | UAAAGAACUCUUAAAACCCAAU | 988 |
| hsa-miR-378b_st | −0.030689576 | 3:10371913-10371969 (+) | 19 | ACUGGACUUGGAGGCAGAA | 989 |
| hsa-miR-3134_st | 0.098733552 | 3:15738805-15738878 (−) | 23 | UGAUGGAUAAAAGACUACAUAUU | 990 |
| hsa-miR-3135_st | −0.442883908 | 3:20179057-20119133 (+) | 22 | UGCCUAGGCUGAGACUGCAGUG | 991 |
| hsa-miR-466_st | −0.028270025 | 3:31203196-31203279 (−) | 23 | AUACACAUACACGCAACACACAU | 992 |
| hsa-miR-3136_st | −0.190142604 | 3:69098109-69098186 (−) | 23 | CUGACUGAAUAGGUAGGGUCAUU | 993 |
| hsa-miR-544b_st | −0.066212259 | 3:124451286-124451363 (+) | 22 | ACCUGAGGUUGUGCAUUUCUAA | 994 |
| hsa-miR-3137_st | −0.548300574 | 3:194855235-194855309 (−) | 24 | UCUGUAGCCUGGGAGCAAUGGGGU | 995 |
| hsa-miR-3138_st | −0.393980701 | 4:10080235-10080316 (−) | 24 | UGUGGACAGUGAGGUAGAGGGAGU | 996 |
| hsa-miR-3139_st | −0.072638653 | 4:144264613-144264688 (+) | 23 | UAGGAGCUCAACAGAUGCCUGUU | 997 |
| hsa-miR-3140_st | −0.149655043 | 4:153410479-153410568 (−) | 22 | AGCUUUUGGGAAUUCAGGUAGU | 998 |
| hsa-miR-548t_st | 0.430239948 | 4:174189311-174189384 (+) | 21 | CAAAAGUGAUCGUGGUUUUUG | 999 |
| hsa-miR-3141_st | −0.544811704 | 5:153975572-153975632 (−) | 19 | GAGGGCGGGUGGAGGAGGA | 1000 |
| hsa-miR-3142_st | 0.17358407 | 5:159901409-159901490 (+) | 22 | AAGGCCUUUCUGAACCUUCAGA | 1001 |
| hsa-miR-3143_st | 0.185111272 | 6:27115405-27115467 (+) | 25 | AUAACAUUGUAAAGCGCUUCUUUCG | 1002 |
| hsa-miR-548u_st | 0.67348033 | 6:57254930-57255010 (+) | 23 | CAAAGACUGCAAUUACUUUUGCG | 1003 |
| hsa-miR-3144- | 0.361121329 | 6:120336325-120336403 (+) | 22 | AGGGGACCAAAGAGAUAUAUAG | 1004 |
| 5p_st | | | | | |
| hsa-miR-3144- | 0.350242292 | 6:120336325-120336403 (+) | 22 | AUAUACCUGUUCGGUCUCUUUA | 1005 |
| 3p_st | | | | | |
| hsa-miR-3145_st | 0.424206114 | 6:138756350-138756431 (−) | 24 | AGAUAUUUUGAGUGUUUGGAAUUG | 1006 |
| hsa-miR- | −0.192757992 | 6:155174494-155174570 (+) | 22 | GGCGACAAAACGAGACCCUGUC | 1007 |
| 1273c_st | | | | | |
| hsa-miR-3146_st | −0.038088065 | 7:19744981-19745059 (−) | 22 | CAUGCUAGGAUAGAAAGAAUGG | 1008 |
| hsa-miR-3147_st | −0.410906459 | 7:57472731-57472796 (+) | 24 | GGUUGGGCAGUGAGGAGGGUGUGA | 1009 |
| hsa-miR-548v_st | 0.284062501 | 8:17539087-17539166 (−) | 22 | AGCUACAGUUACUUUUGCACCA | 1010 |
| hsa-miR-3148_st | −0.283630101 | 8:29814788-29814864 (−) | 22 | UGGAAAAAACUGGUGUGUGCUU | 1011 |
| hsa-miR-3149_st | 0.10201604 | 8:77879004-77879086 (−) | 23 | UUUGUAUGGAUAUGUGUGUGUAU | 1012 |
| hsa-miR-3150_st | −0.557341627 | 8:96085142-96085221 (+) | 22 | CUGGGGAGAUCCUCGAGGUUGG | 1013 |
| hsa-miR-3151_st | −0.63578655 | 8:104166842-104166917 (+) | 21 | GGUGGGGCAAUGGGAUCAGGU | 1014 |
| hsa-miR-3152_st | −0.312469277 | 9:18573304-18573377 (+) | 22 | UGUGUUAGAAUAGGGGCAAUAA | 1015 |
| hsa-miR-3153_st | −0.477067448 | 9:91927140-91927221 (+) | 23 | GGGGAAAGCGAGUAGGGACAUUU | 1016 |
| hsa-miR-3074_st | −0.403860082 | 9:97848296-97848376 (−) | 22 | GAUAUCAGCUCAGUAGGCACCG | 1017 |
| hsa-miR-3154_st | −0.411809229 | 9:131007226-131007309 (−) | 22 | CAGAAGGGGAGUUGGGAGCAGA | 1018 |
| hsa-miR-3155_st | −0.155939504 | 10:6194159-6194240 (+) | 21 | CCAGGCUCUGCAGUGGGAACU | 1019 |
| hsa-miR-3156_st | 0.145082826 | | 22 | AAAGAUCUGGAAGUGGGAGACA | 1020 |
| hsa-miR-3157_st | −0.286881084 | 10:97824072-97824156 (−) | 22 | UUCAGCCAGGCUAGUGCAGUCU | 1021 |
| hsa-miR-3158_st | 0.038133672 | | 22 | AAGGGCUUCCUCUCUGCAGGAC | 1022 |
| hsa-miR-3159_st | −0.238871967 | 11:18409334-18409407 (+) | 22 | UAGGAUUACAAGUGUCGGCCAC | 1023 |
| hsa-miR-3160_st | 0.217559091 | | 22 | AGAGCUGAGACUAGAAAGCCCA | 1024 |
| hsa-miR-3161_st | 0.046100674 | 11:48118334-48118410 (+) | 23 | CUGAUAAGAACAGAGGCCCAGAU | 1025 |
| hsa-miR-3162_st | −0.452127683 | 11:59362550-59362631 (−) | 23 | UUAGGGACUAGAAGGGUGGGGAG | 1026 |
| hsa-miR-3163_st | −0.155224654 | 11:66701905-66701977 (−) | 22 | UAUAAAAUGAGGGCAGUAAGAC | 1027 |
| hsa-miR-3164_st | 0.026347417 | 11:68850644-68850726 (+) | 22 | UGUGACUUUAAGGGAAAUGGCG | 1028 |
| hsa-miR-3165_st | −0.34715416 | 11:71783274-71783348 (−) | 22 | AGGUGGAUGCAAUGUGACCUCA | 1029 |
| hsa-miR-3166_st | 0.144398541 | 11:87909670-87909761 (+) | 23 | CGCAGACAAUGCCUACUGGCCUA | 1030 |
| hsa-miR- | 0.056152106 | 11:96074602-96074690 (+) | 19 | AUCCCACCACUGCCACCAU | 1031 |
| 1260b_st | | | | | |
| hsa-miR-3167_st | 0.046061902 | 11:126858354-126858438 (−) | 22 | AGGAUUUCAGAAAUACUGGUGU | 1032 |
| has-miR-3168_st | −0.043290328 | 13:41675155-41675236 (−) | 17 | GAGUUCUACAGUCAGAC | 1033 |
| has-miR-3169_st | −0.022725278 | 13:61773932-61774014 (−) | 22 | UAGGACUGUGCUUGGCACAUAG | 1034 |
| hsa-miR-3170_st | −0.228412507 | 13:98860778-98860854 (+) | 22 | CUGGGGUUCUGAGACAGACAGU | 1035 |
| hsa-miR-3171_st | −0.327109797 | 14:28102411-28102484 (−) | 24 | AGAUGUAUGGAAUCUGUAUAUAUC | 1036 |
| hsa-miR-3172_st | −0.193826153 | 14:32954301-32954379 (+) | 19 | UGGGGUUUUGCAGUCCUUA | 1037 |
| hsa-miR-3173_st | −0.283616732 | 14:95604256-95604323 (−) | 22 | AAAGGAGGAAAUAGGCAGGCCA | 1038 |
| hsa-miR-1193_st | −0.10471872 | 14:101496389-101496466 (+) | 23 | GGGAUGGUAGACCGGUGACGUGC | 1039 |
| hsa-miR-323b- | −0.229066875 | 14:101522556-101522637 (+) | 23 | AGGUUGUCCGUGGUGAGUUCGCA | 1040 |
| 5p_st | | | | | |
| hsa-miR-323b- | 0.508572443 | 14:101522556-101522637 (+) | 22 | CCCAAUACACGGUCGACCUCUU | 1041 |
| 3p_st | | | | | |
| hsa-miR-3174_st | 0.000583267 | 15:90349987-90550073 (+) | 23 | UAGUGAGUUAGAGAUGCAGAGCC | 1042 |
| hsa-miR-3175_st | −0.155279288 | 15:93447629-93447705 (+) | 22 | CGGGGAGAGAACGCAGUGACGU | 1043 |
| hsa-miR-3176_st | 0.040745882 | 16:593277-593366 (+) | 19 | ACUGGCCUGGGACUACCGG | 1044 |
| hsa-miR-3177_st | −0.41569559 | 16:1784986-1785067 (+) | 21 | UGCACGGCACUGGGGACACGU | 1045 |
| hsa-miR-3178_st | −0.399033874 | 16:2581925-2582006 (−) | 17 | GGGGCGCGGCCGGAUCG | 1046 |
| hsa-miR-3179_st | −0.339389507 | | 22 | AGAAGGGGUGAAAUUUAAACGU | 1047 |
| hsa-miR-3180- | −0.642017769 | | 25 | CUUCCAGACGCUCCGCCCCACGUCG | 1048 |
| 5p_st | | | | | |
| hsa-miR-3180- | −0.720010774 | | 22 | UGGGGCGGAGCUUCCGGAGGCC | 1049 |
| 3p_st | | | | | |
| hsa-miR-548w_st | −0.221954829 | 16:26036558-26036631 (+) | 23 | AAAAGUAACUGCGGUUUUUGCCU | 1050 |
| hsa-miR-3181_st | −0.477664644 | 16:50776216-50776288 (+) | 19 | AUCGGGCCCUCGGCGCCGG | 1051 |
| hsa-miR-3182_st | 0.068447773 | 16:83541951-83542013 (+) | 17 | GCUUCUGUAGUGUAGUC | 1052 |
| hsa-miR-3183_st | −0.022834953 | 17:925716-925799 (−) | 22 | GCCUCUCUCGGAGUCGCUCGGA | 1053 |
| hsa-miR-3184_st | −0.381752425 | 17:28444104-28444178 (−) | 24 | UGAGGGGCCUCAGACCGAGCUUUU | 1054 |
| hsa-miR-3185_st | −0.67158713 | 17:46801770-46801837 (−) | 23 | AGAAGAAGGCGGUCGGUCUGCGG | 1055 |
| hsa-miR-3065- | 1.258036846 | 17:79099677-79099755 (+) | 23 | UCAACAAAAUCACUGAUGCUGGA | 1056 |
| 5p_st | | | | | |
| hsa-miR-3065- | 0.81022744 | 17:79099677-79099755 (+) | 23 | UCAGCACCAGGAUAUUGUUGGAG | 1057 |
| 3p_st | | | | | |
| hsa-miR-3186- | 0.136863218 | 17:79418130-79418214 (−) | 22 | CAGGCGUCUGUCUACGUGGCUU | 1058 |
| 5p_st | | | | | |
| hsa-miR-3186- | −0.28100309 | 17:79418130-79418214 (−) | 21 | UCACGCGGAGAGAUGGCUUUG | 1059 |
| 3p_st | | | | | |
| hsa-miR-3187_st | −0.65937084 | 19:813584-813653 (+) | 20 | UUGGCCAUGGGGCUGCGCGG | 1060 |
| hsa-miR-3188_st | −0.588548166 | 19:18392887-18392971 (+) | 23 | AGAGGCUUUGUGCGGAUACGGGG | 1061 |
| hsa-miR-3189_st | −0.411248984 | 19:18497372-18497444 (+) | 21 | CCCUUGGGUCUGAUGGGGUAG | 1062 |
| hsa-miR-320e_st | −0.061648408 | 19:47212550-47212602 (−) | 18 | AAAGCUGGGUUGAGAAGG | 1063 |
| hsa-miR-3190- | −0.255634524 | 19:47730199-47730278 (+) | 23 | UGUGGAAGGUAGACGGCCAGAGA | 1064 |
| 5p_st | | | | | |
| hsa-miR-3190- | −0.263953125 | 19:47730199-47730278 (+) | 22 | UGGAAGGUAGACGGCCAGAGAG | 1065 |
| 3p_st | | | | | |
| hsa-miR-3191_st | −0.217382811 | 19:47730201-47730276 (−) | 23 | UGGGGACGUAGCUGGCCAGACAG | 1066 |
| hsa-miR-3192_st | −0.231668368 | 20:18451259-18451335 (+) | 23 | UCUGGGAGGUUGUACCAGUGGAA | 1067 |
| hsa-miR-3193_st | −0.032326265 | 20:30194989-30195043 (+) | 22 | UCCUGCGUAGGAUCUGAGGAGU | 1068 |
| hsa-miR-3194_st | −0.404916179 | 20:50069442-50069514 (−) | 21 | GGCCAGCCACCAGAGGGCUG | 1069 |
| hsa-miR-3195_st | −0.584672209 | 20:60639858-60639941 (+) | 17 | CGCGCCGGGCCCGGGUU | 1070 |
| hsa-miR-3196_st | −0.412279642 | 20:61870131-61870194 (+) | 18 | CGGGGCGGCAGGGGCCUC | 1071 |
| hsa-miR-548x_st | 0.16197379 | 21:20058408-20058482 (−) | 21 | UAAAAACUGCAAUUACUUUCA | 1072 |
| hsa-miR-3197_st | −0.248571382 | 21:42539484-42539556 (+) | 23 | GGAGGCGCAGGCUCGGAAAGGCG | 1073 |
| hsa-miR-3198_st | −0.555869902 | 22:18246946-18247025 (−) | 22 | GUGGAGUCCUGGGGAAUGGAGA | 1074 |
| hsa-miR-3199_st | −0.146392966 | | 23 | AGGGACUGCCUUAGGAGAAAGUU | 1075 |
| hss-miR-3200_st | 0.718370764 | 22:31127544-31127628 (+) | 22 | CACCUUGCGCUACUCAGGUCUG | 1076 |
| hsa-miR-3201_st | −0.418632783 | 22:48670176-48670227 (+) | 17 | GGGAUAUGAAGAAAAAU | 1077 |
| hsa-miR-514b- | −0.373860947 | X:146331669-146331748 (−) | 22 | UUCUCAAGAGGGAGGCAAUCAU | 1078 |
| 5p_st | | | | | |
| hsa-miR-514b- | 0.375642672 | X:146331669-146331748 (−) | 21 | AUUGACACCUCUGUGAGUGGA | 1079 |
| 3p_st | | | | | |
| hsa-miR-3202_st | −0.22165488 | | 22 | UGGAAGGGAGAAGAGCUUUAAU | 1080 |
| hsa-miR- | −0.246540664 | 1:10287776-10287861 (+) | 25 | GAACCCAUGAGGUUGAGGCUGCAGU | 1081 |
| 1273d_st | | | | | |
| hsa-miR-4295_st | 0.200141414 | 10:114393929-114394013 (+) | 18 | CAGUGCAAUGUUUUCCUU | 1082 |
| hsa-miR-4296_st | −0.630156939 | 10:126721352-126721439 (−) | 17 | AUGUGGGCUCAGGCUCA | 1083 |
| hsa-miR-4297_st | 0.079300954 | 10:131641563-131641638 (−) | 16 | UGCCUUCCUGUCUGUG | 1084 |
| hsa-miR-378e_st | 0.141493059 | 10:132760851-132760931 (−) | 25 | ACUGGACUUGGAGUCAGAAGAGUGG | 1085 |
| hsa-miR-4293_st | 0.126162345 | 10:14425199-14925276 (−) | 17 | CAGCCUGACAGGAACAG | 1086 |
| hsa-miR-4294_st | −0.276095962 | 10:50193557-50193632 (−) | 17 | GGGAGUCUACAGCAGGG | 1087 |
| hsa-miR-4301_st | −0.094108767 | 11:113320745-113320810 (−) | 20 | UCCCACUACUUCACUUGUGA | 1088 |
| hsa-miR-4299_st | 0.196050419 | 11:11678198-11678269 (−) | 18 | GCUGGUGACAUGAGAGGC | 1089 |
| hsa-miR-4298_st | −0.256083264 | 11:1880694-1880766 (−) | 22 | CUGGGACAGGAGGAGGAGGCAG | 1090 |
| hsa-miR-4300_st | −0.491785636 | 11:81601783-81601878 (−) | 18 | UGGGAGCUGGACUACUUC | 1091 |
| hsa-miR-4304_st | −0.092297363 | 12:123495214-123495275 (−) | 17 | CCGGCAUGUCCAGGGCA | 1092 |
| hsa-miR-4302_st | −0.039688413 | 12:26026953-26027012 (−) | 18 | CCAGUGUGGCUCAGCGAG | 1093 |
| hsa-miR-4303_st | 0.19446374 | 12:98389161-98389226 (−) | 17 | UUCUGAGCUGAGGACAG | 1094 |
| hsa-miR-4305_st | −0.240743153 | 13:40238171-40238272 (−) | 18 | CCUAGACACCUCCAGUUC | 1095 |
| hsa-miR-4306_st | −0.051694064 | 13:100295313-100295403 (+) | 17 | UGGAGAGAAAGGCAGUA | 1096 |
| hsa-miR-4309_st | 0.127149097 | 14:103005981-103006063 (+) | 18 | CUGGAGUCUAGGAUUCCA | 1097 |
| hsa-miR-4307_st | 0.326413052 | 14:27377848-27377931 (+) | 19 | AAUGUUUUUUCCUGUUUCC | 1098 |
| hsa-miR-4308_st | 0.593586623 | 14:55344831-55344911 (−) | 18 | UCCCUGGAGUUUCUUCUU | 1099 |
| hsa-miR-4310_st | 0.575886605 | 15:42158693-42158749 (−) | 16 | GCAGCAUUCAUGUCCC | 1100 |
| hsa-miR-4311_st | −0.283666111 | 15:66332571-66332670 (+) | 18 | GAAAGAGAGCUGAGUGUG | 1101 |
| hsa-miR-4312_st | −0.344771543 | 15:69094189-69094264 (−) | 19 | GGCCUUGUUCCUGUCCCCA | 1102 |
| hsa-miR-4313_st | −0.28819078 | 15:76054556-76054656 (−) | 20 | AGCCCCCUGGCCCCAAACCC | 1103 |
| hsa-miR-4315_st | 0.437871294 | | 18 | CCGCUUUCUGAGCUGGAC | 1104 |
| hsa-miR-4316_st | −0.406166064 | 17:75393066-75393136 (−) | 17 | GGUGAGGCUAGCUGGUG | 1105 |
| hsa-miR-4314_st | −0.264898682 | 17:7991374-7991465 (+) | 18 | CUCUGGGAAAUGGGACAG | 1106 |
| hsa-miR-4318_st | −0.04902337 | 18:35237098-35237178 (+) | 17 | CACUGUGGGUACAUGCU | 1107 |
| hsa-miR-4319_st | −0.180180524 | 18:42550047-42550131 (−) | 17 | UCCCUGAGCAAAGCCAC | 1108 |
| hsa-miR-4320_st | −0.115193904 | 18:47652869-47652933 (−) | 18 | GGGAUUCUGUAGCUUCCU | 1109 |
| hsa-miR-4317_st | 0.194590584 | 18:6374360-6374424 (−) | 17 | ACAUUGCCAGGGAGUUU | 1110 |
| hsa-miR-4322_st | −0.669816339 | 19:10341089-10341161 (+) | 21 | CUGUGGGCUCAGCGCGUGGGG | 1111 |
| hsa-miR-4321_st | −0.846697098 | 19:2250638-2250717 (+) | 21 | UUAGCGGUGGACCGCCCUGCG | 1112 |
| hsa-miR-4323_st | −0.181143271 | 19:42637597-42637665 (−) | 18 | CAGCCCCACAGCCUCAGA | 1113 |
| hsa-miR-4324_st | 0.244952427 | 19:49812054-49812125 (−) | 20 | CCCUGAGACCCUAACCUUAA | 1114 |
| hsa-miR-4256_st | −0.264327488 | 1:113004392-113004455 (−) | 18 | AUCUGACCUGAUGAAGGU | 1115 |
| hsa-miR-4257_st | −0.472791231 | 1:150524405-150524490 (+) | 18 | CCAGAGGUGGGGACUGAG | 1116 |
| hsa-miR-4258_st | −0.545130693 | 1:154948169-154948259 (+) | 17 | CCCCGCCACCGCCUUGG | 1117 |
| hsa-miR-4259_st | −0.53303471 | 1:159869769-159869869 (−) | 22 | CAGUUGGGUCUAGGGGUCAGGA | 1118 |
| hsa-miR-4260_st | −0.308889731 | 1:209796789-209796855 (−) | 19 | CUUGGGGCAUGGAGUCCCA | 1119 |
| hsa-miR-4253_st | −0.667912661 | 1:23189652-23189719 (−) | 18 | AGGGCAUGUCCAGGGGGU | 1120 |
| hsa-miR-4251_st | −0.130602414 | 1:3044539-3044599 (+) | 17 | CCUGAGAAAAGGGCCAA | 1121 |
| hsa-miR-4254_st | −0.043967396 | 1:32224261-32224336 (−) | 23 | GCCUGGAGCUACUCCACCAUCUC | 1122 |
| hsa-miR-4255_st | −0.020830263 | 1:37627164-37627235 (+) | 17 | CAGUGUUCAGAGAUGGA | 1123 |
| hsa-miR-4252_st | −0.057427141 | 1:6489894-6489956 (−) | 19 | GGCCACUGAGUCAGCACCA | 1124 |
| hsa-miR-4325_st | 0.265406105 | 20:55896558-55896647 (−) | 18 | UUGCACUUGUCUCAGUGA | 1125 |
| hsa-miR-4326_st | 0.006635041 | 20:61918160-61918218 (+) | 20 | UGUUCCUCUGUCUCCCAGAC | 1126 |
| hsa-miR-4327_st | −0.64185473 | 21:31747612-31747696 (−) | 19 | GGCUUGCAUGGGGGACUGG | 1127 |
| hsa-miR-4261_st | 0.03213025 | 2:10332740-10332797 (−) | 16 | AGGAAACAGGGACCCA | 1128 |
| hsa-miR-4265_st | −0.144983092 | 2:109757946-109758044 (−) | 19 | CUGUGGGCUCAGCUCUGGG | 1129 |
| hsa-miR-4266_st | −0.67079355 | 2:109930027-109930081 (−) | 16 | CUAGGAGGCCUUGGCC | 1130 |
| hsa-miR-4267_st | 0.001682038 | 2:110827538-110827619 (−) | 16 | UCCAGCUCGGUGGCAC | 1131 |
| hsa-miR-4262_st | 0.150034332 | 2:11977059-11977112 (−) | 17 | GACAUUCAGACUACCUG | 1132 |
| hsa-miR-2355_st | 0.311136818 | 2:207974711-207974797 (−) | 21 | AUCCCCAGAUACAAUGGACAA | 1133 |
| hsa-miR-4268_st | −0.251584982 | 2:220771223-220771286 (−) | 21 | GGCUCCUCCUCUCAGGAUGUG | 1134 |
| hsa-miR-4269_st | −0.178058762 | 2:240227157-240227240 (+) | 21 | GCAGGCACAGACAGCCCUGGC | 1135 |
| hsa-miR-4263_st | 0.41640847 | 2:28219234-28219316 (+) | 18 | AUUCUAAGUGCCUUGGCC | 1136 |
| hsa-miR-4264_st | 0.175115529 | 2:79876420-79876485 (−) | 17 | ACUCAGACAUGGUCAUU | 1137 |
| hsa-miR-4270_st | −0.502606734 | 3:15537746-15537815 (−) | 20 | UCAGGGAGUCAGGGGAGGGC | 1138 |
| hsa-miR-4271_st | −0.446206505 | 3:49311553-49311619 (+) | 19 | GGGGGAAGAAAAGGUGGGG | 1139 |
| hsa-miR-4272_st | −0.166774205 | 3:67275888-67275951 (+) | 18 | CAUUCAACUAGUGAUUGU | 1140 |
| hsa-miR-4273_st | 0.128983317 | 3:75787431-75787514 (+) | 18 | GUGUUCUCUGAUGGACAG | 1141 |
| hsa-miR-4276_st | 0.275823689 | 4:175344946-175345015 (+) | 17 | CUCAGUGACUCAUGUGC | 1142 |
| hsa-miR-4275_st | −0.132958871 | 4:28821204-28821290 (+) | 17 | CCAAUUACCACUUCUUU | 1143 |
| hsa-miR-4274_st | −0.229930196 | 4:7461755-7461845 (+) | 18 | CAGCAGUCCCUCCCCCUG | 1144 |
| hsa-miR-4281_st | −0.516018376 | 5:176056440-176056501 (−) | 18 | GGGUCCCGGGGAGGGGGG | 1145 |
| hsa-miR-4277_st | −0.074151462 | 5:1708900-1708983 (−) | 21 | GCAGUUCUGAGCACAGUACAC | 1146 |
| hsa-miR-4279_st | −0.892166402 | 5:31936208-31936265 (−) | 16 | CUCUCCUCCCGGCUUC | 1147 |
| hsa-miR-4278_st | −0.63517387 | 5:6827966-6828034 (−) | 18 | CUAGGGGGUUUGCCCUUG | 1148 |
| hsa-miR-4280_st | 0.142387426 | 5:86410696-81410771 (−) | 21 | GAGUGUAGUUCUGAGCAGAGC | 1149 |
| hsa-miR-4282_st | 0.153932894 | 6:73677410-73677476 (−) | 18 | UAAAAUUUGCAUCCAGGA | 1150 |
| hsa-miR-4285_st | −0.648225861 | 7:101936369-101936453 (+) | 18 | GCGGCGACUCCGACUCAU | 1151 |
| hsa-miR-4283_st | −0.754587333 | | 17 | UGGGGCUCAGCGAGUUU | 1152 |
| hsa-miR-4284_st | 0.312303346 | 7:73125647-73125727 (+) | 18 | GGGCUCACAUCACCCCAU | 1153 |
| hsa-miR-4286_st | 0.333955292 | 8:10524488-10524580 (+) | 17 | ACCCCACUCCUGGUACC | 1154 |
| hsa-miR-4287_st | 0.328615626 | 8:27743556-27743633 (−) | 19 | UCUCCCUUGAGGGCACUUU | 1155 |
| hsa-miR-4288_st | 1.017740413 | 8:28362633-28362699 (−) | 17 | UUGUCUGCUGAGUUUCC | 1156 |
| hsa-miR-4292_st | −0.394930683 | 9:139725409-139725475 (+) | 18 | CCCCUGGGCCGGCCUUGG | 1157 |
| hsa-miR-4289_st | 0.754785186 | 9:91360751-91360820 (−) | 19 | GCAUUGUGCAGGGCUAUCA | 1158 |
| hsa-miR-4290_st | −0.682599288 | 9:92785723-92785817 (−) | 19 | UGCCCUCCUUUCUUCCCUC | 1159 |
| hsa-miR-4291_st | 0.914392688 | 9:96581639-96581703 (+) | 16 | UUCAGCAGGAACAGCU | 1160 |
| hsa-miR-4329_st | −0.123251977 | X:112023946-112024016 (−) | 19 | CCUGAGACCCUAGUUCCAC | 1161 |
| hsa-miR-4330_st | −0.381432698 | X:150336694-150336798 (+) | 19 | CCUCAGAUCAGAGCCUUGC | 1162 |
| hsa-miR-500b_st | 0.319409972 | X:49775280-49775358 (+) | 19 | AAUCCUUGCUACCUGGGU | 1163 |
| hsa-miR-4328_st | 0.221976157 | X:78156691-78156746 (−) | 17 | CCAGUUUUCCCAGGAUU | 1164 |

A total of 156 human miRNAs were significantly differentially expressed between the Benign and the Malignant groups of samples (p<0.05). Among these, 41 miRNAs were over-expressed (Log 2 diff (Malignant vs Benign)≥1) and 7 miRNAs were under-expressed (Log 2 diff (Malignant vs Benign)≤1) by at least 2-fold in the Malignant group compared to the Benign group of samples, with a p-value≤0.05. Of the miRNAs that were over-expressed, hsa-miR-146b was over-expressed by more than 15-fold in Malignant vs Norm, and hsa-miR-221*, -375, and -31* were over-expressed by more than 5-fold in the Malignant group of lesions compared to the Benign group of specimens (Tables 5A-C). In addition, hsa-miR-31*, -146b-3p, -21, -222, -221, -551b, -34a, and -31, were 3- to 5-fold overexpressed in Malignant lesions compared to Benign specimens (Tables 5A-C). Among the miRNAs that were under-expressed, hsa-miR-7 was under-expressed in the Malignant group by more than 4-fold compared to the Benign group of samples (Tables 5A-C).

TABLE 5A
miRNAs significantly differentially expressed between the groups of Benign and
Malignant samples.

| | AVG | SD | AVE | SD | Fold | |
|---|---|---|---|---|---|---|
| ProbeSet | benign | benign | malignant | malignant | change | P-Value |
| hsa-miR-146b-5p st | 8.65 | 2.78 | 12.65 | 3.18 | 16.0 | 1.31E−05 |
| hsa-miR-221-star st | 5.35 | 2.24 | 8.07 | 1.85 | 6.6 | 8.79E−06 |
| hsa-miR-375 st | 4.40 | 1.60 | 6.83 | 3.47 | 5.4 | 3.40E−03 |
| hsa-miR-31-star st | 2 22 | 1.96 | 4.61 | 3.02 | 5.2 | 1.77E−03 |
| hsa-miR-146B-3p st | 3.91 | 1.05 | 6.12 | 2.62 | 4.6 | 5.30E−04 |
| hsa-miR-21 st | 10.51 | 2.21 | 12.68 | 1.67 | 4.5 | 1.21E−04 |
| hsa-miR-222 st | 11.66 | 2.08 | 13.62 | 1.51 | 3.9 | 1.69E−04 |
| hsa-miR-221 sT | 11.92 | 1.95 | 13.82 | 1.52 | 3.7 | 1.60E−04 |
| hsa-miR-551b st | 2.24 | 2.08 | 4.13 | 3.20 | 3.7 | 1.67E−02 |
| hsa-miR-34a st | 8.64 | 1.94 | 10.28 | 1.97 | 3.1 | 3.28E−03 |
| hsa-miR-31 st | 8.52 | 2.09 | 10.15 | 2.63 | 3.1 | 1.72E−02 |
| hsa-miR-24-2-star st | 4.62 | 1.71 | 6.16 | 1.55 | 2.9 | 9.67E−04 |
| hsa-miR-34a-star st | 4.95 | 1.10 | 6.46 | 1.37 | 2.8 | 6.97E−05 |
| hsa-miR-146a st | 8.00 | 2.54 | 9.49 | 1.68 | 2.8 | 1.18E−02 |
| hsa-miR-340 st | 2.76 | 2.00 | 4.25 | 1.89 | 2.8 | 6.87E−03 |
| hsa-miR-301a st | 3.05 | 2.12 | 4.53 | 2.02 | 2.8 | 1.10E−02 |
| hsa-miR-34b-star st | 3.84 | 1.38 | 5.22 | 2.09 | 2.6 | 8.17E−03 |
| hsa-miR-142-5p st | 3.36 | 2.36 | 4.71 | 2.03 | 2.6 | 2.66E−02 |
| hsa-miR-425 st | 7.68 | 1.89 | 9.00 | 1.47 | 2.5 | 5.10E−03 |
| hsa-miR-27a st | 1122 | 2.32 | 12.53 | 1.27 | 2.5 | 104E−02 |
| hsa-miR-335 st | 7.35 | 2.25 | 8.64 | 2.36 | 2.4 | 4.59E−02 |
| hsa-miR~I82 si | 7.85 | 2.08 | 9.12 | 2.02 | 2.4 | 2.75E−02 |
| hsa-miR-3065-5p st | 2.93 | 1.55 | 4.18 | 1.70 | 2.4 | 6.83E−03 |
| hsa-miR-376c st | 3.57 | 1.13 | 4.79 | 1.87 | 2.3 | 7.57E−03 |
| hsa-miR-181a-star st | 4.45 | 1.39 | 5.66 | 1.91 | 2.3 | 1.20E−02 |
| hsa-miR-10b st | 8.30 | 2.39 | 9.49 | 1.36 | 2.3 | 2.36E−02 |
| hsa-miR-1827 st | 4.49 | 1.20 | 5.67 | 1.27 | 2.3 | 1.08E−03 |
| hsa-miR-493-star st | 3.11 | 0.84 | 4.28 | 0.83 | 2 2 | 4.24E−06 |
| hsa-miR-24-1-star st | 2.80 | 1.64 | 3.95 | 1.95 | 2.2 | 2.45E−02 |
| hsa-miR-329 st | 2.75 | 1.08 | 3.88 | 1.62 | 2 2 | 5.44E−03 |
| hsa-miR-140-5p st | 4.09 | 1.54 | 5.22 | 2.06 | 2.2 | 3.04E−02 |
| hsa-miR-15a st | 9.52 | 2.07 | 10.63 | 158 | 2.2 | 2.87E−02 |
| hsa-miR-1274a st | 9.87 | 1.23 | 10.95 | 1.09 | 2.1 | 1.04E−03 |
| hsa-miR-132 st | 6.10 | 2.05 | 7.16 | 1.50 | 2.1 | 3.02E−02 |
| hsa-miR-4288 st | 3.91 | 1.23 | 4.93 | 1.09 | 2.0 | 2.11E−03 |
| hsa-miR-181 st | 7.71 | 1.79 | 8.73 | 1.83 | 2.0 | 4.52E−02 |
| hsa-miR-495 st | 2.34 | 1.29 | 3.34 | 2.10 | 2.0 | 4.72E−02 |
| hsa-miR-196a_st | 2.21 | 1.15 | 3.20 | 1.14 | 2.0 | 2.40E−03 |
| hsa-miR-132-star_st | 2.37 | 1.49 | 3.37 | 1.63 | 2.0 | 2.38E−02 |
| hsa-miR-224_st | 6.11 | 1.45 | 7.10 | 1.40 | 2.0 | 1.32E−02 |
| hsa-miR-27b-star_st | 5.70 | 1.28 | 6.69 | 1.38 | 2.0 | 9.13E−03 |
| hsa-miR-487b_st | 6.65 | 1.50 | 7.61 | 1.46 | 1.9 | 2.10E−02 |
| hsa-miR-338-5p_st | 4.60 | 1.17 | 5.53 | 1.22 | 1.9 | 6.12E−03 |
| hsa-miR-4291_st | 5.57 | 1.57 | 6.48 | 1.28 | 1.9 | 2.10E−02 |
| hsa-miR-21-star_st | 5.81 | 0.92 | 6.72 | 1.06 | 1.9 | 1.58E−03 |
| hsa-miR-18a_st | 4.14 | 1.10 | 5.03 | 1.26 | 1.9 | 8.62E−03 |
| hsa-miR-16-2-star_st | 3.10 | 1.35 | 3.99 | 1.34 | 1.9 | 1.83E−02 |
| hsa-miR-99a-star_st | 4.07 | 1.47 | 4.95 | 1.27 | 1.8 | 2.11E−02 |
| hsa-miR-24_st | 12.73 | 1.68 | 13.61 | 1.13 | 1.8 | 2.44E−07 |
| hsa-miR-369-5p_st | 2.91 | 1.00 | 3.76 | 1.57 | 1.8 | 2.55E−02 |
| hsa-miR-146a-star_st | 0.60 | 1.29 | 1.44 | 1.31 | 1.8 | 2.13E−02 |
| hsa-miR-3065-3p_st | 3.81 | 1.65 | 4.62 | 1.29 | 1.8 | 4.64E−02 |
| hsa-miR-222-star_st | 5.73 | 0.98 | 6.53 | 1.32 | 1.7 | 1.68E−02 |
| hsa-miR-302b_st | −0.26 | 1.16 | 0.54 | 1.42 | 1.7 | 2.95E−02 |
| hsa-miR-202-star_st | −0.78 | 0.78 | −0.01 | 1.24 | 1.7 | 1.10E−02 |
| hsa-miR-452_st | 5.78 | 1.01 | 6.55 | 1.27 | 1.7 | 1.97E−02 |
| hsa-miR-890_st | 5.26 | 1.20 | 6.02 | 1.07 | 1.7 | 1.64E−02 |
| hsa-miR4289_st | 5.48 | 1.39 | 6.23 | 0.91 | 1.7 | 1.89E−02 |
| hsa-miR-186-star_st | 0.09 | 0.77 | 0.84 | 1.02 | 1.7 | 4.56E−03 |
| hsa-miR-16-1-star_st | 3.00 | 1.07 | 3.74 | 0.98 | 1.7 | 9.93E−03 |
| hsa-miR-3200_st | 3.15 | 0.90 | 3.87 | 1.25 | 1.6 | 2.24E−02 |
| hsa-miR-539_st | 3.11 | 0.94 | 3.80 | 1.39 | 1.6 | 4.42E−02 |
| hsa-miR-548u_st | 1.15 | 1.00 | 1.83 | 1.11 | 1.6 | 2.38E−02 |
| hsa-miR-578_st | 2.87 | 1.03 | 3.53 | 0.99 | 1.6 | 1.95E−02 |
| hsa-miR-376a_st | 3.31 | 0.98 | 3.97 | 1.12 | 1.6 | 2.76E−02 |
| hsa-miR-582-5p_st | 0.88 | 1.27 | 1.53 | 1.09 | 1.6 | 4.51E−02 |
| hsa-miR-1274b_st | 13.41 | 0.98 | 14.06 | 0.79 | 1.6 | 8.53E−03 |
| hsa-miR-323-3p_st | 3.30 | 0.82 | 3.92 | 0.95 | 1.5 | 1.47E−02 |
| hsa-miR-181c-star_st | 6.15 | 1.12 | 6.75 | 1.03 | 1.5 | 4.50E−02 |
| hsa-miR-196a-star_st | 4.14 | 1.05 | 4.71 | 0.79 | 1.5 | 2.42E−02 |
| hsa-miR-642_st | 3.02 | 0.93 | 3.52 | 0.86 | 1.4 | 4.66E−02 |
| hsa-miR-1293_st | 7.78 | 0.67 | 7.44 | 0.53 | 1.3 | 4.06E−02 |
| hsa-miR-1303_st | 10.51 | 0.68 | 10.16 | 0.58 | 1.3 | 4.68E−02 |
| hsa-miR-770-5p_st | 6.62 | 0.65 | 6.22 | 0.73 | 1.3 | 3.77E−02 |
| hsa-miR-3147_st | 11.26 | 0.56 | 10.85 | 0.60 | 1.3 | 1.23E−02 |
| hsa-miR-363-star_st | 10.16 | 0.69 | 9.74 | 0.58 | 1.3 | 1.75E−02 |
| hsa-miR-1229_st | 7.87 | 0.70 | 7.43 | 0.84 | 1.4 | 4.40E−02 |
| hsa-miR-3135_st | 6.39 | 0.72 | 5.95 | 0.66 | 1.4 | 2.09E−02 |
| hsa-miR-423-5p_st | 13.23 | 0.70 | 12.78 | 0.71 | 1.4 | 2.37E−02 |
| hsa-miR-3153_st | 9.39 | 0.67 | 8.92 | 0.73 | 1.4 | 1.61E−02 |
| hsa-miR-296-3p_st | 11.85 | 0.69 | 11.37 | 0.80 | 1.4 | 2.23E−02 |
| hsa-miR-1180_st | 9.65 | 0.72 | 9.17 | 0.86 | 1.4 | 2.92E−02 |
| hsa-miR-1323_st | 7.65 | 0.88 | 7.16 | 0.85 | 1.4 | 3.91E−02 |
| hsa-miR-3131_st | 11.92 | 0.76 | 11.41 | 0.97 | 1.4 | 4.21E−02 |
| hsa-miR-1915-star_st | 7.88 | 0.65 | 7.37 | 0.90 | 1.4 | 2.46E−02 |
| hsa-miR-323-5p_st | 11.03 | 0.85 | 10.52 | 0.98 | 1.4 | 4.77E−02 |
| hsa-miR-194-star_st | 8.34 | 0.90 | 7.83 | 0.85 | 1.4 | 3.37E−02 |
| hsa-miR-658_st | 12.09 | 0.83 | 11.56 | 1.05 | 1.4 | 4.81E−02 |
| hsa-miR-4259_st | 11.02 | 0.90 | 10.48 | 1.02 | 1.4 | 4.85E−02 |
| hsa-miR-1281_st | 9.16 | 0.89 | 8.63 | 1.03 | 1.4 | 4.92E−02 |
| hsa-miR-3130-3p_st | 7.73 | 0.88 | 7.19 | 1.05 | 1.5 | 4.96E−02 |
| hsa-miR-302c-star_st | 7.86 | 1.16 | 7.32 | 0.85 | 1.5 | 4.94E−02 |
| hsa-miR-139-3p_st | 7.69 | 0.83 | 7.15 | 1.00 | 1.5 | 3.56E−02 |
| hsa-miR-1237_st | 8.05 | 0.72 | 7.50 | 0.77 | 1.5 | 9.56E−03 |
| hsa-miR-1181_st | 9.91 | 0.84 | 9.36 | 0.62 | 1.5 | 6.70E−03 |
| hsa-miR-3124_st | 11.72 | 0.99 | 11.16 | 1.03 | 1.5 | 4.86E−02 |
| hsa-miR-3150_st | 9.40 | 0.89 | 8.84 | 1.06 | 1.5 | 4.45E−02 |
| hsa-miR-449b-star_st | 7.94 | 0.69 | 7.38 | 0.94 | 1.5 | 1.87E−02 |
| hsa-miR-574-5p_st | 7.92 | 0.55 | 7.35 | 1.16 | 1.5 | 3.65E−02 |
| hsa-miR-920_st | 10.32 | 0.89 | 9.75 | 1.03 | 1.5 | 3.35E−02 |
| hsa-miR-939_st | 13.40 | 0.82 | 12.82 | 1.03 | 1.5 | 2.91E−02 |
| hsa-miR-92a-1-star_st | 8.02 | 0.80 | 7.44 | 1.04 | 1.5 | 2.8711-02 |
| hsa-miR-3195_st | 12.82 | 0.94 | 12.24 | 1.13 | 1.5 | 4.71E−02 |
| hsa-miR-1207-3p_st | 3.97 | 0.72 | 3.38 | 0.80 | 1.5 | 6.57E−03 |
| hsa-miR-616_st | 7.54 | 0.62 | 6.95 | 0.77 | 1.5 | 3.66E−03 |
| hsa-miR-1470_st | 5.75 | 0.71 | 5.16 | 0.94 | 1.5 | 1.30E−02 |
| hsa-miR-2277_at | 8.84 | 0.83 | 8.24 | 0.97 | 1.5 | 1.93E−02 |
| hsa-miR-1225-3p_st | 8.02 | 1.07 | 7.42 | 0.97 | 1.5 | 3.19E−02 |
| hsa-miR-769-3p_st | 9.67 | 1.03 | 9.06 | 1.01 | 1.5 | 3.18E−02 |
| hsa-miR-661_st | 7.37 | 0.72 | 6.76 | 1.11 | 1.5 | 2.36E−02 |
| hsa-miR-4296_at | 7.28 | 0.76 | 6.64 | 1.08 | 1.5 | 1.94E−02 |
| hsa-miR-1976_at | 7.96 | 0.63 | 7.32 | 0.91 | 1.5 | 6.09E−03 |
| hsa-miR-668_st | 8.41 | 0.74 | 7.78 | 0.93 | 1.5 | 9.40E−03 |
| hsa-miR-4278_at | 6.86 | 1031 | 6.22 | 0.94 | 1.6 | 4.09E−02 |
| hsa-miR-3151_st | 11.33 | 0.82 | 10.69 | 0.97 | 1.6 | 1.31E−02 |
| hsa-miR-4327_at | 12.09 | 0.94 | 11.45 | 1.14 | 1.6 | 3.09E−02 |
| hsa-miR-3180-5p_st | 7.73 | 0.85 | 7.08 | 0.95 | 1.6 | 1.22E−02 |
| hsa-miR-623_st | 9.08 | 0.87 | 8.43 | 1.27 | 1.6 | 3.87E−02 |
| hsa-miR-1909-star_st | 9.07 | 0.67 | 8.42 | 0.84 | 1.6 | 3.27E−03 |
| hsa-miR-3187_st | 12.63 | 1.04 | 11.97 | 1.15 | 1.6 | 3.34E−02 |
| hsa-miR-4253_st | 11.25 | 0.89 | 10.59 | 1.27 | 1.6 | 3.48E−02 |
| hsa-miR-639_st | 8.84 | 0.96 | 8.17 | 1.16 | 1.6 | 2.70E−02 |
| hsa-miR-4322_st | 12.60 | 0.93 | 11.93 | 1.12 | 1.6 | 2.27E−02 |
| hsa-miR-4266_st | 6.15 | 0.81 | 5.48 | 0.99 | 1.6 | 1.01E−02 |
| hsa-miR-760_st | 10.58 | 0.85 | 9.91 | 0.90 | 1.6 | 6.89E−03 |
| hsa-miR-197_st | 7.75 | 0.62 | 7.06 | 1.04 | 1.6 | 6.58E−03 |
| hsa-miR-637_st | 12.60 | 0.95 | 11.90 | 1.06 | 1.6 | 1.48E−02 |
| hsa-miR-1231_st | 13.99 | 1.11 | 13.29 | 1.38 | 1.6 | 4.89E−02 |
| hsa-miR-880-5p_st | 12.24 | 0.69 | 11.53 | 1.43 | 1.6 | 3.20E−02 |
| hsa-miR-3180-3p_st | 14.82 | 1.09 | 14.10 | 1.09 | 1.6 | 1.86E−02 |
| hsa-miR-346_st | 9.25 | 0.69 | 8.52 | 0.81 | 1.7 | 8.64E−04 |
| hsa-miR-935_st | 6.68 | 1.03 | 5.93 | 1.06 | 1.7 | 1.21E−02 |
| hsa-let-7b-star_st | 7.05 | 0.74 | 6.30 | 1.01 | 1.7 | 3.93E−03 |
| hsa-miR-193b-star_st | 11.48 | 0.93 | 10.73 | 0.90 | 1.7 | 3.96E−03 |
| hsa-miR-885-3p_st | 13.74 | 1.10 | 12.99 | 1.15 | 17 | 1.79E−02 |
| hsa-miR-4283_st | 8.71 | 0.82 | 7.95 | 0.83 | 1.7 | 1.50E−03 |
| hsa-miR-371-3p_st | 4.50 | 1.38 | 3.74 | 1.19 | 1.7 | 3.09E−02 |
| hsa-miR-548q_st | 7.91 | 0.63 | 7.14 | 1.07 | 1.7 | 3.47E−03 |
| hsa-miR-596_st | 8.08 | 0.72 | 7.30 | 1.21 | 1.7 | 8.12E−03 |
| hsa-miR-636_st | 9.38 | 0.99 | 8.60 | 1.26 | 1.7 | 1.62E−02 |
| hsa-miR-943_st | 7.96 | 1.10 | 7.16 | 1.16 | 1.7 | 1.18E−02 |
| hsa-miR-193a-5p_st | 12.47 | 0.98 | 11.64 | 1.15 | 1.8 | 6.78E−03 |
| hsa-miR-486-3p_st | 10.16 | 1.03 | 9.32 | 1.22 | 1.8 | 9.94E−03 |
| hsa-miR-4321_st | 10.11 | 0.86 | 9.27 | 1.40 | 1.8 | 1.29E−02 |
| hsa-miR-557_st | 12.05 | 0.96 | 11.21 | 1.48 | 1.8 | 1.98E−02 |
| hsa-miR-4279_st | 5.63 | 0.83 | 4.74 | 1.36 | 1.9 | 7.48E−03 |
| hsa-miR-138-1-star_st | 7.63 | 1.09 | 6.72 | 1.46 | 1.9 | 1.47E−02 |
| hsa-miR-373-star_st | 11.18 | 1.23 | 10.26 | 1.38 | 1.9 | 1.42E−02 |
| hsa-miR-885-5p_st | 7.38 | 1.12 | 6.46 | 1.45 | 1.9 | 1.42E−02 |
| hsa-miR-185-star_st | 9.74 | 0.79 | 8.77 | 1.96 | 2.0 | 2.94E−02 |
| hsa-miR-3123_st | 0.81 | 1.05 | −0.19 | 0.78 | 2.0 | 1.61E−04 |
| hsa-miR-1910_st | 10.20 | 0.67 | 9.17 | 1.01 | 2.0 | 1.04E−04 |
| hsa-miR-139-5p_st | 7.02 | 1.94 | 5.93 | 1.95 | 2.1 | 4.42E−02 |
| hsa-miR-486-5p_st | 8.82 | 1.57 | 7.68 | 1.27 | 2.2 | 4.53E−03 |
| hsa-miR-519a_st | 1.00 | 1.59 | 0.20 | 1.17 | 2.3 | 2.01E−03 |
| hsa-miR-7_st | 8.43 | 2.97 | −6.27 | 3.78 | 4.5 | 2.62E−02 |

AVG, average expression among samples in a group;
SD, standard deviation

TABLE 5B
miRNAs significantly differentially expressed between the groups of Benign and
Malignant samples.

| | | | | SEQ |
|---|---|---|---|---|
| | | | | ID |
| ProbeSet | FDR | Alignments | Sequence | NO: |
| hsa-miR-146b-5p_st | 0.005 | 10:104196269-1041956341 (+) | UGAGAACUGAAUUCCAUAGGCU | 3 |
| hsa-miR-221-star_st | 0.005 | X:45605585-45605694 (−) | ACCUGGCAUACAAUGUAGAUUU | 7 |
| hsa-miR-375_st | 0.159 | 2:219866367-219866430 (−) | UUUGUUCGUUCGGCUCGCGUGA | 13 |
| hsa-miR-31-star_st | 0.114 | 9:21512114-21512184 (−) | UGCUAUGCCAACAUAUUGCCAU | 41 |
| hsa-miR-146b-3p_st | 0.058 | 10:104196269-104196341 (+) | UGCCCUGUGGACUCAGUUCUGG | 4 |
| hsa-miR-21_st | 0.021 | 17:57918627-57918698 (+) | UAGCUUAUCAGACUGAUGUUGA | 22 |
| hsa-miR-222_st | 0.021 | X:45606421-45606530 (−) | AGUACAUCUGGCUACUGGGU | 31 |
| hsa-miR-221_st | 0.021 | X:4560585-45605694 (−) | AGCUACAUUGUCUGCUGGGUUUC | 17 |
| hsa-miR-51b_st | 0.246 | 3:168269642-168269737 (+) | GCGACCCAUACUUGGUUUCAG | 54 |
| hsa-miR-34a_st | 0.159 | 1:9211727-9211836 (−) | UGGCAGUGUCUUAGCUGGUUGU | 23 |
| hsa-miR-31_st | 0.250 | 9:21512114-21512184 (−) | AGGCAAGAUGCUGGCAUAGCU | 18 |
| hsa-miR-24-2-star_st | 0.085 | | UGCCUACUGAGCUGAAACACAG | 38 |
| hsa-miR-34a-star_st | 0.019 | 1:9211727-9211836 (−) | CAAUCAGCAAGUAUACUGCCCU | 42 |
| hsa-miR-146a_st | 0.221 | 5:159912359-159912457 (+) | UGAGAACUGAAUUCCAUGGGUU | 34 |
| hsa-miR-340_st | 0.189 | 5:179442303-179442397 (−) | UUAUAAAGCAAUGAGACUGAUU | 53 |
| hsa-miR-301a_st | 0.221 | 17:57228497-57228582 (−) | CAGUGCAAUAGUAUUGUCAAAGC | 51 |
| hsa-miR-34b-star_st | 0.204 | 11:111383663-111383746 (+) | UAGGCAGUGUCAUUAGCUGAUUG | 43 |
| hsa-miR-142-5p_st | 0.274 | 17:56408593-56408679 (−) | CAUAAAGUAGAAAGCACUACU | 46 |
| hsa-miR-425_st | 0.187 | 3:49057581-49057667 (−) | AAUGACACGAUCACUCCCGUUGA | 33 |
| hsa-miR-27a_st | 0.215 | 19:13947254-13947331 (−) | UUCACAGUGGCUAAGUUCCGC | 39 |
| hsa-miR-335_st | 0.350 | 7:130135952-130136045 (+) | UCAAGAGCAAUAACGAAAAAUGU | 52 |
| hsa-miR-182_st | 0.276 | 7:129410223-129410332 (−) | UUUGGCAAUGGUAGAACUCACACU | 49 |
| hsa-miR-3065-5p_st | 0.189 | 17:79099677-79099755 (+) | UCAACAAAAUCACUGAUGCUGGA | 56 |
| hsa-miR-376c_st | 0.198 | 14:101506027-101506092 (+) | AACAUAGAGGAAAUUCCACGU | 11 |
| hsa-miR-181a-star_st | 0.221 | | ACCAUCGACCGUUGAUUGUACC | 47 |
| hsa-miR-10b_st | 0.261 | 2:177015031-177015140 (+) | UACCCUGUAGAACCGAAUUUGUG | 35 |
| hsa-miR-1827_st | 0.085 | 12:100583662-100583727 (+) | UGAGGCAGUAGAUUGAAU | 55 |
| hsa-miR-493-star_st | 0.005 | 14:101335397-101335485 (+) | UUGUACAUGGUAGGCUUUCAUU | 19 |
| hsa-miR-24-1-star_st | 0.261 | | UGCCUACUGAGCUGAUAUCAGU | 37 |
| hsa-miR-329_st | 0.189 | | AACACACCUGGUUAACCUCUUU | 5 |
| hsa-miR-140-5p_st | 0.283 | 16:69966984-69967083 (+) | CAGUGGUUUUACCCUAUGGUAG | 250 |
| hsa-miR-15a_st | 0.280 | 13:50623255-50623337 (−) | UAGCAGCACAUAAUGGUUUGUG | 36 |
| hsa-miR-1274a_st | 0.085 | 5:41475734-41475804 (+) | GUCCCUGUUCAGGCGCCA | 64 |
| hsa-miR-132_st | 0.283 | 17:1953202-1953302 (−) | UAACAGUCUACAGCCAUGGUCG | 44 |
| hsa-miR-4288_st | 0.122 | 8:28362633-28362699 (−) | UUGUCUGCUGAGUUUCC | 57 |
| hsa-miR-181c_st | 0.350 | 19:13985513-13985622 (+) | AACAUUCAACCUGUCGGUGAGU | 48 |
| hsa-miR-495_st | 0.350 | 14:101500092-101500173 (+) | AAACAAACAUGGUGCACUUCUU | 9 |
| hsa-miR-196a_st | 0.132 | | UAGGUAGUUUCAUGUUGUUGGG | 50 |
| hsa-miR-132-star_st | 0.261 | 17:1953202-1953302 (−) | ACCGUGGCUUUCGAUUGUUACU | 45 |
| hsa-miR-224_st | 0.223 | X:151127050-151127130 (4 | CAAGUCACUAGUGGUUCCGUU | 32 |
| hsa-miR-27b-star_st | 0.214 | 9:97847727-97847823 (+) | AGAGCUUAGCUGAUUGGUGAAC | 40 |
| hsa-miR-487b_st | 0.255 | 14:101512792-101512875 (+) | AAUCGUACAGGGUCAUCCACUU | 557 |
| hsa-miR-538-5p_st | 0.189 | 17:79099683-79099749 (−) | AACAAUAUCCUGGUGCUGAGUG | 397 |
| hsa-miR-4291_st | 0.255 | 9:96581639-96581703 (441 | UUCAGCAGGAACAGCU | 1160 |
| hsa-miR-21-star_st | 0.109 | 17:57918627-57918698 (+) | CAACACCAGUCGAUGGGCUGU | 95 |
| hsa-miR-18a_st | 0.206 | 13:92003005-92003075 (+) | UAAGGUGCAUCUAGUGCAGAUAG | 85 |
| hsa-miR-16-2-star_st | 0.350 | | CCAAUAUUACUGUGCUGCUUUA | 148 |
| hsa-miR-99a-star_st | 0.255 | 21:17911409-17911489 (+) | CAAGCUCGCUUCUAUGGGUCUG | 133 |
| hsa-miR-24_st | 0.261 | | UGGCUCAGUUCAGCAGGAACAG | 101 |
| hsa-miR-369-5p_st | 0.267 | 14:101531935-101532004 (+) | AGAUCGACCGUGUUAUAUUCGC | 15 |
| hsa-miR-146a-star_st | 0.255 | 5:159912359-159912457 (+) | CCUCUGAAAUUCAGUUCUUCAG | 281 |
| hsa-miR-3065-3p_st | 0.350 | 17:79099677-79099755 (4) | UCAGCACCAGGAUAUUGUUGGAG | 1057 |
| hsa-miR-222-star_st | 0.146 | X:45606421-45606530 (−) | CUCAGUAGCCAGUGUAGAUCCU | 14 |
| hsa-miR-302b_st | 0.180 | 4:113569641-113569713 (−) | UAAGUGCUUCCAUGUUUAGUAG | 343 |
| hsa-miR-202-star_st | 0.221 | 10:135061015-135061124 (−) | UUCCUAUGCAUAUACUUCUUUG | 455 |
| hsa-miR-452_st | 0.250 | X:151128100-151128184 (−) | AACUGUUUGGAGAGGAAACUGA | 430 |
| hsa-miR-890_st | 0.246 | X:145075793-145075869 (−) | UACUUGGAAAGGCAUCAGUUG | 753 |
| hsa-miR-4289_st | 0.250 | 9:91360751-91360820 (−) | GCAUUGUGCAGGGCUAUCA | 1158 |
| hsa-miR-186-star_st | 0.173 | 1:71533314-71533399 (−) | GCCCAAAGGUGAAUUUUUUGGG | 292 |
| hsa-miR-16-1-star_st | 0.214 | | CCAGUAUUAACUGUGCUGCUGA | 82 |
| hsa-miR-3200_st | 0.261 | 22:31127544-31127628 (+) | CACCUUGCGCUACUCAGGUCUG | 21 |
| hsa-miR-539_st | 0.350 | 14:101513658-101513735 (+) | GGAGAAAUUAUCCUUGGUGUGU | 1 |
| hsa-miR-548a_st | 0.261 | 6:57254930-57255010 (+) | CAAAGACUGCAAUUACUUUUGCG | 1003 |
| hsa-miR-578_st | 0.250 | 4:166307394-166507489(f) | CUUCUUGUGCUCUAGGAUUGU | 590 |
| hsa-miR-376a 81 | 0.276 | | AUCAUAGAGGAAAAUCCACGU | 363 |
| hsa-miR-582-511_st | 0.350 | 5:58999432-58999529 (−) | UUACAGUUGUUCAACCAGUUACU | 594 |
| hsa-miR-1274b_st | 0.206 | 19:58024375-58024441 (−) | UCCCUGUUCGGGCGCCA | 899 |
| hsa-miR-323-3p_st | 0.232 | 14:101492069-101492154 (+) | CACAUUACACGGUCGACCUCU | 385 |
| hsa-miR-181c-star_st | 0.350 | 19:13985513-13985622 (+) | AACCAUCGACCGUUGAGUGGAC | 181 |
| hsa-miR-196a-star_st | 0.261 | | CGGCAACAAGAAACUGCCUGAG | 188 |
| hsa-miR-642_st | 0.350 | 19:46178186-461782112 (+) | GUCCCUCUCCAAAUGUGUCUUG | 673 |
| hsa-miR-1293_st | 0.336 | 12:50627925-50627995 (−) | UGGGUGGUCUGGAGAUUUGUGC | 845 |
| hsa-miR-1303_st | 0.350 | 5:154065336-154065421 (+) | UUUAGAGACGGGGUCUUGCUCU | 852 |
| hsa-miR-770-5p_st | 0.319 | 14:101318727-101318824 (+) | UCCAGUACCACGUGUCAGGGCCA | 740 |
| hsa-miR-3147_st | 0.221 | 7:57472731-57472796 (+) | GGUUGGGCAGUGAGGAGGGUGUGA | 1009 |
| hsa-miR-363-star_st | 0.250 | X:133303408-133303482 (−) | CGGGUGGAUCACGAUGCAAUUU | 338 |
| hsa-miR-1229_st | 0.350 | 5:179225278-179225346 (−) | CUCUCACCACUGCCCUCCCACAG | 818 |
| hsa-miR-3135_st | 0.255 | 3:20179057-20179133 (+) | UGCCUAGGCUGAGACUGCAGUG | 991 |
| hsa-miR-423-5p_st | 0.261 | 17:28444097-28444190 (+) | UGAGGGGCAGAGAGCGAGACUUU | 411 |
| hsa-miR-3153_st | 0.246 | 9:91927140-91927221 (+) | GGGGAAAGCGAGUAGGGACAUUU | 1016 |
| hsa-miR-296-3p_st | 0.261 | 20:57392670-57392749 (−) | GAGGGUUGGGUGGAGGCUCUCC | 328 |
| hsa-miR-1180_st | 0.280 | 17:19247819-19247887 (−) | UUUCCGGCUCGCGUGGGUGUGU | 806 |
| hsa-miR-1323_st | 0.326 | 19:54175222-554175294 (+) | UCAAAACUGAGGGGCAUUUUCU | 719 |
| hsa-miR-3131_st | 0.343 | 2:219923410-219923472 (−) | UCGAGGACUGGUGGAAGGGCCUU | 986 |
| hsa-miR-1915-star_st | 0.261 | 10:21785491-21785570 (−) | ACCUUGCCUUGCUGCCCGGGCC | 941 |
| hsa-miR-323-5p_st | 0.350 | 14:101492069-101492154 (+) | AGGUGGUCCGUGGCGCGUUCGC | 384 |
| hsa-miR-194-star_st | 0.294 | | CCAGUGGGGCUGCUGUUAUCUG | 307 |
| hsa-miR-658_st | 0.350 | 22:38240279-38240378 (−) | GGCGGAGGGAAGUAGGUCCGUUGGU | 700 |
| hsa-miR-4259_st | 0.350 | 1:159869769-159869869 (−) | CAGUUGGGUCUAGGGGUCAGGA | 118 |
| hsa-miR-1281_st | 0.350 | 22:41488517-41488570 (+) | UCGCCUCCUCCUCUCCC | 900 |
| hsa-miR-3130-3p_st | 0.350 | | GCUGCACCGGAGACUGGGUAA | 985 |
| hsa-miR-302c-staR_st | 0.350 | 4:113569519-413569586 (−) | UUUAACAUGGGGGUACCUGCUG | 344 |
| hsa-miR-159-3p_st | 0.306 | 11:72326107-72326174 (−) | GGAGACGCGGCCCUGUUGGAGU | 166 |
| hsa-miR-1237_st | 0.214 | 11:64136074-64 36175 (+) | UCCUUCUGCUCCGUCCCCCAG | 823 |
| hsa-miR-1181_st | 0.189 | 19:10514134-10514214 (−) | CCGUCGCCGCCACCCGAGCCG | 807 |
| hsa-miR-3124_st | 0.350 | 1:249120576-249120642 (+) | UUCGCGGGCGAAGGCAAAGUC | 976 |
| hsa-miR-3150_st | 0.350 | 8:96035142-96085221 (+) | CUGGGGAGAUCCUCGAGGUUGG | 1013 |
| hsa-miR-449B-star_st | 0.250 | 5:54466474-54466570 (−) | CAGCCACAACUACCCUGCCACU | 690 |
| hsa-miR-574-5p_st | 0.311 | 4:38869653-38869748 (+) | UGAGUGUGUGUGUGUGAGUGUGU | 584 |
| hsa-miR-920_st | 0.294 | 12:24365355-24365429 (+) | GGGGAGCUGUGGAAGCAGUA | 786 |
| hsa-miR-939_st | 0.280 | 8:145619364-145619445 (−) | UGGGGAGCUGAGGCUCUGGGGGUG | 797 |
| hsa-miR-92a-1-star_st | 0.280 | | AGGUUGGGAUCGGUUGCAAUGCU | 123 |
| hsa-miR-3195_st | 0.350 | 20:60639858-60639941 (+) | CGCGCCGGGCCCGGGUU | 1070 |
| hsa-miR-1207-3p_st | 0.189 | 8:129061398429061484 (+) | UCAGCUGGCCCUCAUUUC | 834 |
| hsa-miR-616_st | 0.161 | 12:57912946-57913042 (−) | AGUCAUUGGAGGGUUUGAGCAG | 639 |
| hsa-miR-1470_st | 0.223 | 19:1556035945560419 (+) | GCCCUCCGCCCGUGCACCCCG | 921 |
| hsa-miR-2277_st | 0.250 | 5:92936402-92956494 (−) | UGACAGCGCCCUGCCUGGCUC | 961 |
| hsa-miR-1225-3p_st | 0.286 | 16:2140196-2140285 (−) | UGAGCCCCUGUGCCGCCCCCAG | 812 |
| hsa-miR-769-3p_st | 0.286 | 19:46522190-46522307 (+) | CUGGGAUCUCCGGGGUCUUGGUU | 729 |
| hsa-miR-661_st | 0.261 | 8:145019359-145019447 (−) | UGCCUGGGUCUCUGGCCUGCGCGU | 686 |
| hsa-miR-4296_st | 0.250 | 10:126721352-126121439 (−) | AUGUGGGCUCAGGCUCA | 1083 |
| hsa-miR-1976_st | 0.189 | 1:26881033-26881084 (+) | CCUCCUGCCCUCCUUGCUGU | 946 |
| hsa-miR-668_st | 0.214 | 14:101521595-101521660 (+) | UGUCACUCGGCUCGGCCCACUAC | 710 |
| hsa-miR-4278_st | 0.336 | 5:6827966-6828034 (−) | CUAGGGGGUUUGCCCUUG | 1148 |
| hsa-miR-3151_st | 0.223 | 8:104166842-104166917 (+) | GGUGGGGCAAUGGGAUCAGGU | 1014 |
| hsa-miR-4327_st | 0.283 | 21:31747612-31747696 (−) | GGCUUGCAUGGGGGACUGG | 1127 |
| hsa-miR-3180-5p_st | 0.221 | | CUUCCAGACGCUCCGCCCCACGUCG | 1048 |
| hsa-miR-623_st | 0.325 | 13:100008385-100008482 (+) | AUCCCUUGCAGGGGCUGUUGGGU | 648 |
| hsa-miR-1909-star_st | 0.159 | 19:1816158-1816237 (−) | UGAGUGCCGGUGCCUGCCCUG | 932 |
| hsa-miR-3187_st | 0.294 | 19:813584-813653 (+) | UUGGCCAUGGGGCUGCGCGG | 1060 |
| hsa-miR-4253_st | 0.302 | 1:23189652-23189719 (−) | AGGGCAUGUCCAGGGGGU | 1120 |
| hsa-miR-639_st | 0.275 | 19:14640355-14640452 (+) | AUCGCUGCGGUUGCGAGCGCUGU | 670 |
| hsa-miR-4322_st | 0.261 | 19:10341089-10341161 (+) | CUGUGGGCUCAGCGCGUGGGG | 1111 |
| hsa-miR-4266_st | 0.214 | 2:109930027-109930081 (−) | CUAGGAGGCCUUGGCC | 1130 |
| hsa-miR-760_st | 0.189 | 1:94312388-94312467 (+) | CGGCUCUGGGUCUGUGGGGA | 782 |
| hsa-miR-197_st | 0.189 | 1:110141515-110141589 (+) | UUCACCACCUUCUCCACCCAGC | 30 |
| hsa-miR-637_st | 0.232 | 19:3961412-3961510 (−) | ACUGGGGGCUUUCGGGCUCUGCGU | 668 |
| hsa-miR-1231_st | 0.350 | 1:201777739-201777830 (+) | GUGUCUGGGCGGACAGCUGC | 819 |
| hsa-miR-886-5p_st | 0.286 | 5:135416177-135416297 (−) | CGGGUCGGAGUUAGCUCAAGCGG | 746 |
| hsa-miR-3180-3p_st | 0.250 | | UGGGGCGGAGCUUCCGGAGGCC | 1049 |
| hsa-miR-346_st | 0.085 | 10:88024451-88024545 (−) | UGUCUGCCCGCAUGCCUGCCUCU | 16 |
| hsa-miR-935_st | 0.221 | 19:54485561-54485651 (+) | CCAGUUACCGCUUCCGCUACCGC | 793 |
| hsa-let-7b-star_st | 0.161 | 22:46509566-46509648 (+) | CUAUACAACCUACUGCCUUCCC | 69 |
| hsa-miR-193b-star_st | 0.161 | 16:14397824-14397906 (+) | CGGGGUUUUGAGGGCGAGAUGA | 465 |
| hsa-miR-885-3p_st | 0.250 | 3:10436173-10436246 (−) | AGGCAGCGGGGUGUAGUGGAUA | 773 |
| hsa-miR-4283_st | 0.109 | | UGGGGCUCAGCGAGUUU | 1152 |
| hsa-miR-371-3p_st | 0.283 | 19:54290929-54290995 (+) | AAGUGCCGCCAUCUUUUGAGUGU | 355 |
| hsa-miR-548q_st | 0.159 | 10:12767253-12767352 (−) | GCUGGUGCAAAAGUAAUGGCGG | 959 |
| hsa-miR-596_st | 0.204 | 8:1765397-1765473 (+) | AAGCCUGCCCGGCUCCUCGGG | 616 |
| hsa-miR-636_st | 0.246 | 17:74732532-74732630 (−) | UGUGCUUGCUCGUCCCGCCCGCA | 667 |
| hsa-miR-943_st | 0.221 | 4:1988111-1988204 (−) | CUGACUGUUGCCGUCCUCCAG | 801 |
| hsa-miR-193a-5p_st | 0.189 | 17:29887015-29887102 (+) | UGGGUCUUUGCGGGCGAGAUGA | 296 |
| hsa-miR-486-3p_st | 0.214 | 8:41517959-41718026 (−) | CGGGGCAGCUCAGUACAGGAU | 443 |
| hsa-miR-4321_st | 0.223 | 19:2250638-2250717 (+) | UUAGCGGUGGACCGCCCUGCG | 1112 |
| hsa-miR-557_st | 0.250 | 1:168344762-168344859 (+) | GUUUGCACGGGUGGGCCUUGUCU | 567 |
| hsa-miR-4279_st | 0.198 | 5:31936208-31936265 (−) | CUCUCCUCCCGGCUUC | 1147 |
| hsa-miR-138-1-star_st | 0.232 | | GCUACUUCACAACACCAGGGCC | 24 |
| hsa-miR-373-star_st | 0.232 | 19:54291959-54292027 (+) | ACUCAAAAUGGGGGCGCUUUCC | 357 |
| hsa-miR-885-5p_st | 0.232 | 3:10436173-10436246 (−) | UCCAUUACACUACCCUGCCUCU | 772 |
| hsa-miR-185-star_st | 0.280 | 22:20020662-20020743 (+) | AGGGGCUGGCUUUCCUCUGGUC | 58 |
| hsa-miR-3123_st | 0.021 | 1:241295572-241295646 (+) | CAGAGAAUUGUUUAAUC | 59 |
| hsa-miR-1910_st | 0.021 | 16:85775227-85775306 (−) | CCAGUCCUGUGCCUGCCGCCU | 8 |
| hsa-miR-139-5p_st | 0.350 | 11:72326107-72326174 (−) | UCUACAGUGCACGUGUCUCCAG | 25 |
| hsa-miR-486-5p_st | 0.173 | 8:41517959-41518026 (−) | UCCUGUACUGAGCUGCCCCGAG | 61 |
| hsa-miR-519a_st | 0.122 | | AAAGUGCAUCCUUUUAGAGUGU | 62 |
| hsa-miR-7_st | 0.272 | | UGGAAGACUAGUGAUUUUGUUGU | 2 |

TABLE 5C
FNA Set 2 Model Logistic Weight

| Predictor | Coefficient |
|---|---|
| (Intercept) | 1.759924 |
| Diff(miR-141,miR-7) | −0.27689 |
| Diff(miR-375,miR-7) | −0.16405 |
| Diff(miR-138-1#,miR-155) | −0.11432 |
| Diff(miR-138-1#,miR-7) | −0.10224 |
| Diff(miR-31,miR-7) | −0.02884 |
| Diff(miR-141,miR-155) | −0.01636 |
| Diff(miR-155,miR-31) | 0.0037 |
| Diff(miR-155,miR-375) | 7.54E−16 |

### Example 3

### qRT-PCR Experiments and Data Analysis Methods for the Development of a miRNA Classifier

Samples and qRT-PCR experiments: Two sets of FNA specimens were used for the development of miRNA classifier. The first set (Set 1) contained a total of 14 samples corresponding to 8 benign and 6 malignant specimens, respectively, as described in Table 3A. qRT-PCR reactions were performed using TaqMan MicroRNA Assays (Applied Biosystems; Foster City, Calif., USA) according to the manufacturer's instructions. Reactions were done in duplicates and included 5 ng of total nucleic acids (TNA) per reaction. The reactions were incubated in the 7900HT Fast Real-Time PCR System (Applied Biosystems). The miRNAs tested for Set 1 were: miRs-138-1#, -139-5, -146b-3p, -146b-5p, -21, -222, -320a, -34a, -375, -423-5p, -625#, -7, -126, -143, -144, -193a-3p, -193a-5p, -195, -204, -222#, -320a, -451, -486-3p, and miR-486-5p. These miRNAs were selected based on array analysis of the FFPE samples, as well as additional information obtained from previous work on thyroid tissues using fresh frozen biopsies.

TABLE 6
(former Table *5): qRT-PCR data obtained using the first set (Set1) as a training set for
the development of the miRNA classifier.

| miRNAs | NTC | AVG_Benign | SD_Benign | AVG_Malignant | SD_Malignant |
|---|---|---|---|---|---|
| miR-625# | 31.58 | 29.07 | 1.09 | 29.27 | 0.19 |
| miR-423-5p | 40.00 | 29.47 | 1.47 | 29.20 | 0.47 |
| miR-320a | 40.00 | 24.73 | t.66 | 25.26 | 0.40 |
| miR-126 | 40.00 | 24.72 | 2.06 | 25.29 | 0.73 |
| miR-138-1# | 39.25 | 32.94 | 1.02 | 32.45 | 1.02 |
| miR-139-5p | 3851 | 30.51 | 1.86 | 29.77 | 0.45 |
| miR-143 | 40.00 | 36.31 | 1.04 | 35.35 | 0.74 |
| miR-144 | 37.51 | 28.61 | 2.84 | 30.19 | 1.12 |
| miR-146b- | 40.00 | 35.62 | 0.90 | 29.82 | 2.86 |
| 3p | | | | | |
| miR-146b- | 40.00 | 28.48 | 1.85 | 22.94 | 3.10 |
| 5p | | | | | |
| miR-193a-3p | 40.00 | 35.41 | 1.05 | 34.83 | 0.62 |
| miR-193a-5p | 40.00 | 30.55 | 1.17 | 29.67 | 0.88 |
| miR-195 | 40.00 | 27.03 | 2.04 | 27.68 | 0.51 |
| miR-204 | 40.00 | 31.59 | 1.90 | 31.50 | 1.53 |
| miR-21 | 40.00 | 27.20 | 1.86 | 26.51 | 1.20 |
| miR-222# | 40.00 | 35.82 | 1.58 | 32.49 | 0.73 |
| miR-222 | 40.00 | 25.56 | 1.41 | 22.37 | 0.93 |
| miR-34a | 40.00 | 36.79 | 1.03 | 35.13 | 0.47 |
| rniR-375 | 40.00 | 35.29 | 2.33 | 27.80 | 1.66 |
| miR-451 | 40.00 | 23.17 | 2.61 | 24.09 | 0.98 |
| miR-486-3p | 39.46 | 26.61 | 2.14 | 27.26 | 0.86 |
| miR-486-5p | 40.00 | 20.50 | 2.28 | 21.65 | 1.35 |
| miR-7 | 40.00 | 29.74 | 1.67 | 31.12 | 1.84 |

NTC, no template control.
The miRNAs indicated in italics were used as putative normalizers.

The second set of FNAs (Set 2) contained a total of 21 samples corresponding to a benign group of 5 samples in which no mutation had been identified, a benign group of 5 samples in which mutations had been identified, and a group of 5 malignant samples in which no mutation had been identified and a group of 5 malignant samples in which mutations had been identified, with the exclusion of BRAF mutation. An additional malignant sample was used that contained a BRAF V600E mutation (Table 3B). The miRNAs tested in the second FNA set were miRs-138-1#, -139-5, -146b-3p, -146b-5p, -21, -222, -320a, -34a, -375, -423-5p, -625#, -7, -141, -145, -155, -183, -197, -224, -31, and miR-425-5p. These miRNAs were selected based on array analysis of the FFPE samples excluding BRAF-positive FFPE tumors, as well as additional information obtained from previous work on thyroid tissues using fresh frozen biopsies and data recently published in the literature (Kitano et al., 2011; Nikiforova. et al., 2009).

TABLE 7
(former Table *6): qRT-PCR data obtained using the first set (Set2) as a training set
for the development of the miRNA classifier.

| miRNAs | NTC | AVG_Benign | SD_Benign | AVG_Malignant | SD_Malignant |
|---|---|---|---|---|---|
| miR-625# | 31.38 | 28.66 | 1.56 | 28.72 | 1.10 |
| miR-423-5p | 40.00 | 27.36 | 2.76 | 27.73 | 1.83 |
| miR-320 | 40.00 | 23.33 | 2.90 | 23.91 | 2.00 |
| miR-138-1# | 33.98 | 33.40 | 2.64 | 32.05 | 2.31 |
| miR-139-5p | 40.00 | 31.24 | 3.38 | 30.75 | 3.36 |
| miR-141 | 40.00 | 30.85 | 4.09 | 28.42 | 2.79 |
| miR-145 | 35.26 | 30.20 | 3.05 | 30.00 | 2.19 |
| miR-146b-3p | 40.00 | 34.92 | 2.00 | 32.92 | 3.57 |
| miR-146b-5p | 40.00 | 28.32 | 2.87 | 26.83 | 3.56 |
| miR-155 | 40.00 | 30.03 | 3.44 | 30.71 | 2.70 |
| miR-183 | 40.00 | 29.50 | 3.12 | 30.00 | 2.35 |
| miR-197 | 40.00 | 28.52 | 2.56 | 28.45 | 2.01 |
| miR-21 | 40.00 | 25.12 | 2.23 | 25.56 | 2.30 |
| miR-222 | 40.00 | 26.19 | 2.82 | 25.27 | 2.70 |
| miR-224 | 40.00 | 33.90 | 2.13 | 33.88 | 2.51 |
| miR-31 | 40.00 | 31.05 | 3.56 | 29.22 | 2.91 |
| rniR-34a | 35.47 | 29.03 | 2.02 | 27.61 | 2.16 |
| miR-375 | 40.00 | 33.81 | 4.02 | 30.82 | 3.87 |
| miR-425-5p | 40.00 | 26.10 | 3.29 | 26.46 | 2.41 |
| miR-7 | 40.00 | 27.56 | 2.69 | 29.73 | 4.32 |

NTC, no template control.
The miRNAs indicated in italics were used as putative normalizers.

### DiffPair Normalization

DiffPairs, normalized biomarkers whose values are defined as the difference in expression values (Ct values for qRT-PCR data) between two distinct miRNAs, were used as the normalization method for qRT-PCR data. DiffPair normalization is similar to normalization using a specified normalizer reference gene with the modification that different DiffPairs use different reference genes. Because the DiffPair itself is regarded as a biomarker, DiffPair normalization does not require the assumption that either miRNA composing a given DiffPair is stably expressed across all samples, making DiffPair normalization appropriate in some conditions for which use of fixed normalizer genes can be problematic.

### Logistic Regression for Construction of Classifier

L1-penalized regression (Tibshirani 1996, Goeman 2010), as implemented by the R package penalized, was used to fit logistic model classifier to DiffPaired FNA data sets. The use of an L1-penalty term integrates feature selection, whereby only those DiffPairs who combine to produce the best fitting model without requiring large model weights are allowed to be used as model predictors, directly into the model-training process in order to prevent statistical over-fitting. The L1-penalty used for model-fitting was always fixed at a constant value based on prior assessment of reasonable values for logistic regression models using DiffPaired qRT-PCR data. Performance of this logistic regression modeling methodology was assessed using 5-fold cross-validation (Hastie 2009).

### Example 4

### Sample Information.

We tested an independent set of 65 thyroid FFPE specimens (Set 2) corresponding to 29 benign and 36 malignant neoplasms. The benign samples corresponded to 9 hyperplastic nodules (HN), 8 follicular adenomas (FA), 3 oncocytic follicular adenoma (OFA), 4 multi nodular goiters (MNG), and 5 Hashimoto's thyroiditis (HASH). The malignant samples corresponded to 19 papillary carcinomas (PTC), 8 follicular variant of papillary carcinomas (FVPTC), and 9 follicular carcinomas (FTC). Information regarding these samples is provided in Table X. Total RNA was isolated from each sample, using the RecoverAll RNA Isolation Kit (Ambion Austin Tex., USA). Total RNA was quantified using a NanoDrop® ND-1000 spectrophotometer (NanoDrop Technologic Inc, Wilmington, Del., USA). The samples were tested for mutations and translocations, using Asuragen miRInform® Thyroid Marker Panel. The mutational results of the assay are indicated in Table X.

TABLE X
Patient information and histopathological data for the second set (Set2) of thyroid FFPE samples.

| | | | | | | | | AJCC/ | |
|---|---|---|---|---|---|---|---|---|---|
| | | | | | | | AJCC/ | UICC | |
| Sample | Pathology- | | | | | Excision | UICC | Stage | miRInform ® |
| ID | (Abbr) | Histopathology | Age | Sex | Ethnicity | Year | Stage | Group | Thyroid |
| AF117 | PTC | Papillary | 38 | F | AA | 1995 | T2N1MX | I | BRAF V600E |
| | | carcinoma | | | | | | | |
| AF118 | FTC | Follicular | 47 | F | C | 1995 | T2NIXMX | II | PAX8/PPARg |
| | | carcinoma | | | | | | | |
| AF119 | MNG | Goiter, | 44 | F | C | 1996 | T1bN1MX | III | Neg |
| | | multinodular | | | | | | | |
| AF120 | FTC | Follicular | 46 | F | C | 1996 | T3NXMX | III | Neg |
| | | carcinoma | | | | | | | |
| AF121 | FA | Follicular adenoma | 47 | F | C | 1996 | T2NXMX | II | Neg |
| AF122 | Hash | Follicular Adenoma | 52 | F | C | 2006 | T2NXM0 | II | Neg |
| AF123 | FA | Follicular Adenoma | 41 | F | C | 2007 | T3NXM0 | I | Neg |
| AF124 | NH | Nodular | 54 | M | C | 2009 | N/A | N/A | Neg |
| | | Hyperplasia | | | | | | | |
| AF125 | PTC | Papillary | 24 | M | C | 2007 | T3N1MX | I | BRAF V600E |
| | | Carcinoma | | | | | | | |
| AF126 | PTC | Papillary | 45 | F | C | 2008 | T3NXM0 | III | Neg |
| | | Carcinoma | | | | | | | |
| AF127 | NH | Nodular | 88 | F | C | 2009 | N/A | N/A | Neg |
| | | Hyperplasia | | | | | | | |
| AF128 | FvPTC | Papillary | 38 | F | C | 2009 | T2NXMX | I | Neg |
| | | Carcinoma, | | | | | | | |
| | | Follicular Variant | | | | | | | |
| AF129 | FTC | Follicular | 52 | F | C | 2010 | T2NXM0 | II | Neg |
| | | carcinoma | | | | | | | |
| AF130 | PTC | Papillary | 49 | F | C | 2009 | T3N0M0 | III | Neg |
| | | carcinoma | | | | | | | |
| AF131 | PTC | Papillary | 40 | F | C | 2009 | T2N0M0 | I | Neg |
| | | carcinoma | | | | | | | |
| AF132 | FTC | Follicular | 79 | F | C | 2009 | T1N1M0 | III | Neg |
| | | Carcinoma | | | | | | | |
| AF133 | FA | Hyperplasia | 70 | M | C | 2009 | N/A | N/A | Neg |
| AF134 | PTC | Papillary | 53 | F | C | 2009 | T2N0M0 | II | BRAF V600E |
| | | carcinoma | | | | | | | |
| AF135 | MNG | Goiter, | 73 | M | C | 2009 | N/A | N/A | Neg |
| | | multinodular | | | | | | | |
| AF136 | PTC | Papillary | 77 | N | AP | 2011 | T3NXM0 | III | Neg |
| | | carcinoma | | | | | | | |
| AF137 | PTC | Papillary | 24 | M | AP | 2011 | T3NXM0 | I | BRAF V600E |
| | | carcinoma | | | | | | | |
| AF138 | PTC | Papillary | 19 | F | AP | 2011 | T1bNXM0 | I | BRAF V600E |
| | | carcinoma | | | | | | | |
| AF139 | PTC | Papillary | 67 | F | AP | 2011 | T3NXM0 | III | BRAF V600E |
| | | carcinoma | | | | | | | |
| AF140 | NH | Nodular | 68 | F | C | 2008 | T3NXM0 | III | Neg |
| | | Hyperplasia | | | | | | | |
| AF141 | PTC | Papillary | 58 | F | C | 1994 | T3NXMX | III | BRAF V600E |
| | | carcinoma | | | | | | | |
| AF142 | FA | Follicular adenoma | 36 | M | C | 1994 | T2NXMX | I | Neg |
| AF143 | oFA | Hurthle cell | 52 | M | C | 1994 | T3NXMX | III | Neg |
| | | adenoma | | | | | | | |
| AF144 | FvPTC | Papillary | 29 | F | C | 1994 | T3NXMX | I | BRAF V600E |
| | | carcinoma, | | | | | | | |
| | | follicular variant | | | | | | | |
| AF145 | FA | Follicular adenoma | 38 | F | C | 1994 | N/A | N/A | Neg |
| AF146 | FTC | Follicular | 67 | M | C | 1993 | T3NXMX | III | HRAS Q61R |
| | | carcinoma | | | | | | | |
| AF147 | PTC | Papillary | 54 | F | C | 2008 | T2NXM0 | II | Neg |
| | | Carcinoma | | | | | | | |
| AF148 | NH | nodular | 66 | M | C | 2004 | N/A | N/A | Neg |
| | | hyperplasia | | | | | | | |
| AF149 | NH | Nodular | 45 | M | C | 2004 | N/A | N/A | Neg |
| | | Hyperplasia | | | | | | | |
| AF150 | NH | Nodular | 46 | F | C | 2004 | N/A | N/A | Neg |
| | | Hyperplasia | | | | | | | |
| AF151 | FTC | Follicular | 61 | F | C | 1994 | TXNXMX | | Neg |
| | | carcinoma | | | | | | | |
| AF152 | FA | Follicular adenoma | 65 | F | C | 1995 | N/A | N/A | Neg |
| AF153 | FTC | Follicular | 46 | F | C | 1997 | T2NXMX | | PAX/PPARG |
| | | carcinoma | | | | | | | |
| AF154 | Hash | Hashimoto's | 48 | F | C | 1995 | N/A | N/A | Neg |
| | | thyroiditis | | | | | | | |
| AF156 | NH | Nodular | 29 | F | C | 2004 | T1N0M0 | I | Neg |
| | | Hyperplasia | | | | | | | |
| AF157 | FA | Follicular Adenoma | 47 | M | C | 2006 | T2NXMX | II | Neg |
| AF158 | Hash | Hashimoto's | 81 | M | C | 1995 | T2N0MX | II | Neg |
| | | thyroiditis | | | | | | | |
| AF159 | Hash | Hashimoto's | 45 | F | AP | 1995 | T1aN0MX | I | Neg |
| | | thyroiditis | | | | | | | |
| AF160 | PTC | Papillary | 37 | F | C | 1996 | T2NXMX | I | BRAF V600E |
| | | carcinoma | | | | | | | |
| AF161 | FA | Follicular adenoma | 31 | F | C | 1996 | T2NXMX | I | Neg |
| AF162 | NH | Nodular | N/A | N/A | N/A | N/A | N/A | N/A | Neg |
| | | Hyperplasia | | | | | | | |
| AF163 | FTC | Follicular | 75 | F | BAA | 1994 | T3NXMX | III | NRAS Q61K |
| | | carcinoma | | | | | | | |
| AF164 | FA | Follicalar adenoma | 44 | M | C | 1994 | N/A | N/A | Neg |
| AF165 | FTC | Follicular | 60 | F | C | 1995 | T4aNXMX | IVA | Neg |
| | | carcinoma | | | | | | | |
| AF166 | oFA | Hurthle cell | 42 | F | C | 1995 | N/A | N/A | Neg |
| | | adenoma | | | | | | | |
| AF167 | MNG | Goiter, | 46 | F | C | 1995 | N/A | N/A | Neg |
| | | multinodular | | | | | | | |
| AF168 | MNG | Goiter, | 36 | F | C | 1995 | T2NXMX | I | Neg |
| | | multinodular | | | | | | | |
| AF169 | Hash | Hashimoto's | 36 | F | C | 1995 | N/A | N/A | Neg |
| | | thyroiditis | | | | | | | |
| AF170 | FvPTC | Papillary | 35 | M | C | 1997 | T2NXMX | | Neg |
| | | carcinoma, | | | | | | | |
| | | follicular variant | | | | | | | |
| AF171 | FvPTC | Papillary | F | C | 60 | 1997 | T4NXMX | | HRAS Q61L |
| | | carcinoma, | | | | | | | |
| | | follicular variant | | | | | | | |
| AF172 | PTC | Papillary | 52 | F | C | 2005 | T3N1aM0 | III | Neg |
| | | Carcinoma | | | | | | | |
| AF173 | FvPTC | Papillary | 52 | F | C | 2002 | T2NXM0 | II | Neg |
| | | Carcinoma, | | | | | | | |
| | | Follicular Variant | | | | | | | |
| AF174 | PTC | Papillary | N/A | N/A | N/A | N/A | N/A | N/A | BRAF V600E |
| | | Carcinoma | | | | | | | |
| AF175 | PTC | Papillary | 24 | M | C | 1997 | T3N1MX | I | Neg |
| | | carcinoma | | | | | | | |
| AF176 | FvPTC | Papillary | 40 | F | C | 1996 | T2NXMX | I | BRAF V600E |
| | | carcinoma, | | | | | | | |
| | | follicular variant | | | | | | | |
| AF177 | FvPTC | Papillary | 53 | F | C | 1997 | N/A | N/A | Neg |
| | | carcinoma, | | | | | | | |
| | | follicular variant | | | | | | | |
| AF178 | FvPTC | Papillary | 63 | M | C | 2001 | N/A | N/A | BRAF V600E |
| | | Carcinoma, | | | | | | | |
| | | Follicular Variant | | | | | | | |
| AF179 | PTC | Papillary | 52 | F | C | 2006 | T1NXMX | I | Neg |
| | | Carcinoma | | | | | | | |
| AF180 | PTC | Papillary | 46 | F | C | 2006 | T1NXMX | I | Neg |
| | | Adenocarcinoma | | | | | | | |
| AF181 | PTC | Papillary | 62 | F | C | 2006 | T3NXM0 | III | BRAE V600E |
| | | Carcinoma | | | | | | | |
| AF183 | Hash | Hashimoto's | 73 | F | C | 1996 | T1aN0MX | I | neg |
| | | thyroiditis | | | | | | | |

Anaplastic cancer (ATC);
Follicular Adenoma (FA);
oncocytic FA (OFA);
Follicular carcinoma (FTC);
Papillary carcinoma (PTC);
Follicular variant of PTC (FvPTC),
Hashimoto's thyroiditis (HASH);
multinodular goiter (MNG);
hyperplastic nodules (HN);
Age at Excision (Age);
Male (M);
Female (F);
African-American (AA);
Black/African-American (BAA);
Caucasian (C);
Asian/Pacific (AP)

miRNA Profiling Analysis.

A total of 65 FFPE samples from 29 benign and 36 malignant neoplasms were profiled using the Affymetrix GeneChip® miRNA Arrays V2 in Asuragen Services and then analyzed by Asuragen's bioinformatics group (Asuragen). The Affymetrix miRNA arrays contain probes for more than 1,000 human miRNAs from Sanger miRbase V11.0 (on the World Wide Web at asuragen.com/Services/services). A total of 200 ng of total RNA corresponding to each of the 65 thyroid FFPE was labeled as described in the materials and methods. The thyroid samples were separated in 2 different groups for analysis, corresponding to Benign (hyperplastic nodules (HN; n=9), follicular adenoma (FA; n=8), oncocytic follicular adenoma (OFA; n=3), multi nodular goiter (MNG; n-4), and Hashimoto's thyroiditis (HASH; n==5)) and Malignant (papillary carcinomas (PTC; n=19), follicular variant of papillary carcinomas (FvPTC; n=8), and follicular carcinomas (FTC; n=9). The average levels of expression of each miRNA in each subset of the different groups of samples are shown in Table Y, which is provided at the end of the description.

miRNAs can Distinguish Benign and Malignant Thyroid Neoplasms

A total of 410 human miRNAs were significantly differentially expressed between the Benign and the Malignant groups of samples (p<0.05). Among these, 115 miRNAs were over-expressed (Log 2 diff (Malignant vs Benign)≥1) by at least 2-fold in the Malignant group compared to the Benign group of samples, with a p-value≤0.05. Of these, hsa-miR-146b-5p was over-expressed by more than 30-fold in Malignant vs Benign, and hsa-miR-146b-3p and miR-375 were over-expressed between 9- and 15-fold in the Malignant group of lesions compared to the Benign group of specimens (Table Y). In addition, hsa-miR-221, -221*, -222, -31*, -181a-2*, -551b, -424, -31, -21, and -429 were overexpresssed between 5- and 9-fold in Malignant lesions compared to Benign specimens (Table Y). Only one miRNA, miRNA-7, was down-regulated by at least 2-fold in Malignant compared to Benign lesions (Table Y).

As follicular thyroid cancers are more difficult to diagnose than papillary thyroid cancers, we also compared the miRNA expression profiles of the Follicular thyroid cancer (FTC) group of samples with those of the Benign group of samples. A total of 195 human miRNAs were significantly differentially expressed between the FTC and the Benign groups of samples (p<0.05). Among these, 66 miRNAs were over-expressed (Log 2 diff (FTC vs Benign)≥1) by at least 2-fold in the FTC group compared to the Benign group of samples, with a p-value≤0.05. Of these, two miRNAs, hsa-miR-96 and -182, were over-expressed by more than 5-fold. In FTC vs Benign (Table Y). There was no miRNA down-regulated by at least 2-fold in FTC compared to Benign lesions (Table Y).

A particularly challenging diagnostic issue is to distinguish Follicular carcinoma from Follicular adenoma due to overlapping cytological features between the two entities. We therefore also compared the expression profiles of the Follicular thyroid cancer (FTC) group of samples with those of Follicular adenoma (FA). A total of 101 human miRNAs were significantly differentially expressed between the FTC and the FA groups of samples (p<0.05). Among these, 22 miRNAs were over-expressed (Log 2 diff (FTC vs Benign)≥1) by at least 2-fold in the FTC group compared to the FA. group of samples, with a p-value≤0.05. Of these, two miRNAs, hsa-miR-146b-5p and -181a-2*, were over-expressed by more than 3-fold in FTC vs FA, and 20 miRNAs were over-expressed between 2- and 3-fold in FTC compared to FA (Table Y). There was no miRNA down-regulated by at least 2-fold in FTC compared to FA lesions (Table Y).

Hashimoto's lesions represent also a challenging diagnostic issue. We compared the miRNA expression profiles of the Hashimoto's group of samples (Hash) with those of other benign conditions. A total of 108 human miRNAs were significantly differentially expressed between the Hash and the Benign groups of samples (p<0.05). Among these, 8 miRNAs were over-expressed (Log 2 diff (Hash vs Benign)≥1) by at least 2-fold in the lash group compared to the other Benign group of samples, with a p-value≤0.05. Of these, one miRNA, hsa-miR-142-5p was over-expressed by more than 5-fold in Hash vs Benign. In addition, seven miRNAs, hsa-miR-150, -146b-5p, -155, -146a, -223, -199b-5p, and -31 were over-expressed between 2- and 5-fold in Hash compared to Benign (Table Y). A total of 12 miRNAs were under-expressed in Hash vs Benign by more than 2-fold (Log 2 diff (Hash vs Benign)≤1). Among these, only one miRNA, hsa-miR-7, was down-regulated by more than 5-fold in Hash vs Benign and 11 other miRNas were under-expressed by at least 2-fold in Hash samples compared to the Benign specimens (Table Y). The same comparison was made between Hash samples and the Malignant group of Thyroid specimens. A total of 350 human, miRNAs were significantly differentially expressed between the Hash and the Malignant groups of samples (p<0.05). Among these, 27 miRNAs were over-expressed (Log 2 diff (Hash vs Malignant)≥1) by at least 2-fold in the Hash group compared to the Malignant group of samples, with a p-value≤0.05 (Table Y). A total of 126 miRNAs were under-expressed in Hash vs Malignant by more than 2-fold (Log 2 diff (Hash vs Malignant)≤1). Among these, three miRNAs, hsa-miR-146b-3p, -200b, and -181a-2*, were down-regulated by at least 10-fold in Hash samples compared to the Malignant specimens (Table Y). A total of 21 miRNAs were down-regulated between. 5- and 10-fold in the Hash versus the Malignant groups of specimens, and 102 miRNAs were under-regulated between 2- and 5-fold in the Hash compared to the Malignant groups of samples (Table Y).

### Other Set of FFPE Samples (Set3)

A set of 41 FFPE samples containing 21 malignant and 20 benign thyroid neoplasms including 6 Hashimoto's specimens, was also analyzed to further to build a classifier that. can identify Hashimoto's disease among other benign and malignant thyroid conditions. Information regarding the samples is provided in Table Z1. Total nucleic acids (TNA) were isolated from each sample, using the RecoverAll RNA Isolation Kit (Ambion; Austin, Tex., USA), Total RNA was quantified using a NanoDrop® ND-1000 spectrophotometer (NanoDrop Technologie Inc, Wilmington, Del., USA). The samples were tested for mutations and translocations, using Asuragen miRInform® Thyroid Marker Panel. The mutational results of the assay are indicated in Table Z1. These samples were analyzed by RT-qPCR, as described below.

TABLE Z1
Histopathological information and mutation status for the third set of FFPE
samples (Set3)

| Sample ID | Histopathology | miRinform ® Thyroid |
|---|---|---|
| AF184 | TcPTC | Neg |
| AF185 | PTC | BRAF V600E |
| AF186 | PTC | BRAF V600E |
| AF187 | TcPTC | BRAF V600E |
| AF188 | Hashimoto's | Neg |
| AF189 | Hashimoto's | Neg |
| AF190 | Hashimoto's | BRAF V600E |
| AF191 | Hashimoto's | Neg |
| AF192 | FA | Neg |
| AF193 | FA | Neg |
| AF194 | FA | Neg |
| AF195 | FA | Neg |
| AF196 | FA | Neg |
| AF197 | PTC | Neg |
| AF198 | PTC | Neg |
| AF199 | PTC | Neg |
| AF200 | FA | BRAF V600E |
| AF201 | FA | Neg |
| AF202 | FvPTC | BRAF V600E |
| AF203 | FvPTC | Neg |
| AF204 | PTC | Neg |
| AF205 | FvPTC | Neg |
| AF206 | PTC | HRAS Q61R |
| AF207 | PTC | Neg |
| AF208 | PTC | NRAS Q61R |
| AF209 | PTC | Neg |
| AF210 | PTC | Neg |
| AF211 | PTC | BRAF V600E |
| AF212 | PTC | Neg |
| AF213 | MNG | BRAF V600E |
| AF214 | MNG | Neg |
| AF215 | MNG | BRAF V600E |
| AF216 | MNG | Neg |
| AF217 | MNG | Neg |
| AF218 | MNG | Neg |
| AF219 | MNG | Neg |
| AF220 | Hashimoto's | Neg |
| AF221 | Hashimoto's | Neg |
| AF222 | TcPTC | Neg |
| AF223 | PTC | Neg |
| AF224 | PTC | Neg |

Abbreviations:
Papillary carcinoma (PTC);
Follicular variant of PTC (FvPTC),
Tall cell variant of PTC (TcPTC);
Hashimoto's thyroiditis (HASH);
multinodular goiter (MNG);
Follicular adenoma (FA)

Constrained Logistic Regression Method for Constructing miRNA-Based Classifiers.

A variety of different models can be employed to evaluate expression. levels and/or other comparative values based on expression levels of miRNAs (or their precursors or targets). In particular, a logistic regression model (see the Wikipedia entry on the World Wide Web at en.wikipedia.org/wiki/Logistic_regression, which is hereby incorporated by reference) distinguishing between two diagnostic groups consists of a set of predictor variables, Xᵢ for i between 1 and n together with a set of weight coefficients wᵢ for i between 0 and n, from which the probability that a sample with predictor values Xᵢ=xᵢ is in the first diagnostic group can be computed as

$${P}_{malignant} = \frac{1}{1 + exp ( - {w}_{0} - {\sum }_{i = 1}^{n} {w}_{i} {x}_{i} )}$$

Other examples of models include but are no limited to decision trees, linear or quadratic discriminant analysis, neural networks, support vector machines, and k-nearest neighbor classifiers. A person of ordinary skill in the art could use these different modeling procedures to evaluate expression level data and comparative data involving expression. levels of one or miRNAs (or their precursors or their targets).

Because of the difficulties involved in precisely controlling the amount of intact RNA input for qRT-PCR assays, it is generally desirable to construct models which take as inputs comparative differences in expression between two or more biomarkers instead of the raw cycle threshold (Ct) values measured for individual miRNA biomarkers. One method for accomplishing this is to consider a DiffPair consisting of two biomarkers A. and B associated with the value computed as the difference in Ct value between marker A and marker B (i.e, if x_{A} is the Ct value of marker A and x_{B} is the Ct value of marker B, then x_{A}-x_{B} is the value of the DiffPair Diff(A,B)).

In fitting logistic regression models, an alternative method for adjusting for potential differential intact RNA input levels to the DiffPair method described above is to constrain the sum of the weight coefficients wᵢ for i>0 to be equal to zero:

$${\sum }_{i = 1}^{n} {w}_{i} = 0$$

The result of fitting such as a constrained logistic regression model is that, as in the case with DiffPair values, the model output scores are insensitive to any changes to biomarker Ct values that change the measured Ct values of all predictors upwards or downwards by the same amount, so that only relative expression levels between multiple biomarkers are used by the resulting model to predict the probability of malignancy. Note that in the case of exactly two biomarkers, this constrained logistic regression model becomes a logistic regression model built on a single DiffPair. More generally, models built with this type of constraint can be equivalently described in terms of an unconstrained logistic regression model built using a set of DiffPairs for more than two biomarkers as well (although this may increase the complexity of modeling process).

The classifier algorithms presented below were constructed using only subsets of the singleplex-measured miRNA biomarkers. For each model, the strategy for selection of the miRNA subset used by that model was to choose biomarkers using an unconstrained L2-penalized stepwise logistic regression strategy (L2 penalty parameter set in all cases to λ₂=2.5). Once the subset of biomarkers to be used as predictors for a given model had been identified, the final model was constructed by fitting using the constrained logistic regression described above (again using an L2-penalty λ2=2.5) to the selected predictors. Classifier performance was estimated using leave-one-out-cross-validation evaluating the entire modeling process, including feature selection.

### Results for Constrained Logistic Regression Classifier to Identify Hashimoto's Disease

The intercept (w₀) and weight coefficients (wᵢ for i>0) for the constrained logistic regression classifier for Hashimoto's disease are indicated in [symbol] .

TABLE DX

| Predictor | Coefficient |
|---|---|
| (Intercept) | −8.95 |
| miR-155-5p | −0.93 |
| miR-31-5p | −0.26 |
| miR-151-3p | 0.35 |
| miR-224-5p | 0.84 |

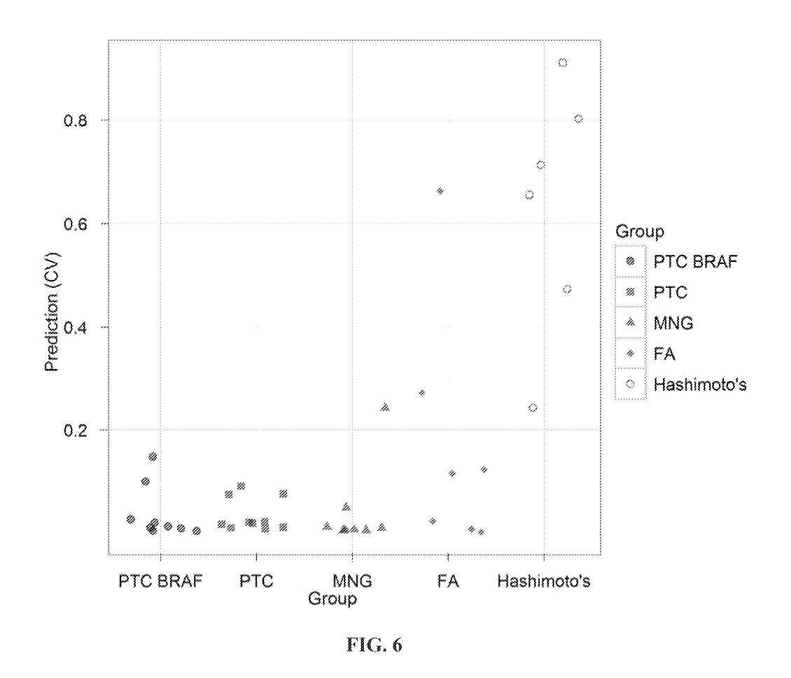

As estimated by leave-one-out-cross-validation (LOOCV) for the modeling strategy described above, the observed accuracy was 93% (95% CI: 80%-98%), sensitivity 67% (95% CI: 22%-96%), specificity 97% (95% CI: 85-100%), with an estimated AUC of 0.98. The confidence interval for sensitivity is very wide because the data set used to train this classifier contained only 6 Hashimoto's samples. The LOOCV classification scores produced by this model are shown in FIG. 6. This classifier is presented as a preliminary example of the type of classifier we intend to develop for the task of separating Hashimoto's disease samples from other thyroid samples. MiR-155, which showed elevated expression in Hashimoto's samples as compared with samples of other diagnostic groupings, is assigned the most negative coefficient in this model. The most positive model coefficient is assigned to miR-224, which exhibits reduced expression in Hashimoto's samples.

### FNA Samples

A total of 62 FNA specimens were analyzed. These samples were classified as benign or malignant, according to their cytology and/or histology report, as described in Table W1. Molecular testing was performed using Asuragen miRInform Thyroid mutation panel assay (Table W1). FNA specimens, corresponding to an entire extra-pass, were collected according to the institutional IRB-approved protocol as a part of standard clinical care, and all samples were de-identified. Upon collection, FNAs were placed in 1 mL of Asuragen's RNARetain™ nucleic acid stabilization solution and shipped to Asuragen at room temperature for molecular testing. Total RNA was extracted using a modified mirVana PARIS™ procedure and proprietary Asuragen protocols optimized for recovery of nucleic acids from RNARetain. Concentration and purity of RNA were measured using the NanoDrop ND-1000 Spectrophotometer (NanoDrop Technologies Inc, Wilmington, Del., USA).

TABLE W1
Classification of the FNAs used in the third set (Set3) of samples. The
samples were classified in two or three categories, according to their
cytological and histological reports. For cytology, the Bethesda
classification was used, and the samples were classified as Benign,
Malignant, Indeterminate, or Non-diagnostic, according to their cytology
reports. The samples were classified according to their histology reports
as Benign or Malignant, and as Benign* when the cytology was benign
and no surgery had been performed after 6 months to 2 years of
follow-up. The mutational status of each sample is indicated, as
determined by the miRInform ® ThyroidPanel Assay.
Papillary thyroid cancer (PTC); Follicular variant of papillary thyroid
cancer (FvPTC). Neg in the mutation column indicates that no mutation
or translocation was found in the sample.

| Sample ID | Cytology categories | Histology categories | Mutation status |
|---|---|---|---|
| FNA-T1 | Malignant | Malignant | Neg |
| FNA-T2 | Malignant | Malignant | Neg |
| FNA-T3 | Malignant | Malignant | Neg |
| FNA-T4 | Malignant | Malignant | Neg |
| FNA-T5 | Malignant | Malignant | Neg |
| FNA-T6 | Indeterminate | Malignant | Neg |
| FNA-T7 | Malignant | Malignant | Neg |
| FNA-T8 | Malignant | Malignant | Neg |
| FNA-T9 | Indeterminate | Malignant | Neg |
| FNA-T10 | Malignant | Malignant | BRAF V600E |
| FNA-T11 | Non diagnostic | Malignant | BRAF V600E |
| FNA-T12 | Malignant | Malignant | BRAF V600E |
| FNA-T13 | Malignant | Malignant | BRAF V600E |
| FNA-T14 | Malignant | Malignant | BRAF V600E |
| FNA-T15 | Benign | Malignant | NRAS Q61R |
| FNA-T16 | Malignant | Malignant | Neg |
| FNA-T17 | Malignant | Malignant | BRAF V600E |
| FNA-T18 | Malignant | Malignant | BRAF V600E |
| FNA-T19 | Malignant | Malignant | BRAF V600E |
| FNA-T20 | Benign | Benign* | Neg |
| FNA-T21 | Malignant | Malignant | BRAF V600E |
| FNA-T22 | Malignant | Malignant | BRAF V600E |
| FNA-T23 | Malignant | Malignant | BRAF V600E |
| FNA-T24 | Benign | Benign* | Neg |
| FNA-T25 | Benign | Benign* | Neg |
| FNA-T26 | Benign | Benign* | Neg |
| FNA-T27 | Benign | Benign* | Neg |
| FNA-T28 | Indeterminate | Benign | Neg |
| FNA-T29 | Benign | Benign* | Neg |
| FNA-T30 | Benign | Benign* | Neg |
| FNA-T31 | Benign | Benign* | Neg |
| FNA-T32 | Indeterminate | Benign | Neg |
| FNA-T33 | Benign | Benign | Neg |
| FNA-T34 | Benign | Benign* | Neg |
| FNA-T35 | Benign | Benign* | Neg |
| FNA-T36 | Benign | Benign* | Neg |
| FNA-T37 | Benign | Benign* | Neg |
| FNA-T38 | Benign | Benign* | Neg |
| FNA-T39 | Benign | Benign* | Neg |
| FNA-T40 | Benign | Benign* | Neg |
| FNA-T41 | Benign | Benign* | Neg |
| FNA-T42 | Malignant | Malignant | BRAF V600E |
| FNA-T43 | Malignant | Malignant | BRAF V600E |
| FNA-T44 | Malignant | Malignant | BRAF V600E |
| FNA-T45 | Malignant | Malignant | BRAF V600E |
| FNA-T46 | Malignant | Malignant | BRAF V600E |
| FNA-T47 | Malignant | Malignant | RET/PTC1 |
| FNA-T48 | Malignant | Malignant | BRAF V600E |
| FNA-T49 | Malignant | Malignant | BRAF V600E |
| FNA-T50 | Malignant | Malignant | BRAF V600E |
| FNA-T51 | Malignant | Malignant | BRAF V600E |
| FNA-T52 | Malignant | Malignant | BRAF V600E |
| FNA-T53 | Benign | Benign* | Neg |
| FNA-T54 | Benign | Benign* | Neg |
| FNA-T55 | Benign | Benign* | Neg |
| FNA-T56 | Benign | Benign* | Neg |
| FNA-T57 | Indeterminate | Malignant | HRAS Q61R |
| FNA-T58 | Indeterminate | Malignant | NRAS Q61R |
| FNA-T59 | Malignant | Malignant | BRAF V600E |
| FNA-T60 | Benign | Benign* | Neg |
| FNA-T61 | Benign | Benign* | Neg |
| FNA-T62 | Benign | Benign* | Neg |

Real-Time Assays and miRNA Classifiers

Real-time RT-qPCR reactions were performed for the 26 candidate miRNAs described in Table O (hsa-miR-375, -146b-3p, -146b-5p, -221, -222, -551b, -204, -7, -141, -31, -181a*-2, -15a, -221*, -21, -424, -34a, -197, -19b, -138-1*, -139-5p, -155, -151-3p, -29b, -96, -328, and 29b-1*). RT-qPCR reactions were performed in duplicates, using Exiqon miRCURY LNA™ Universal RT microRNA PCR Assays (Exiqon A/S, Denmark), using an optimizedversion of the manufacturer's protocol. Reactions included either 20 ng (FFPE samples) or 50 ng (FNAs) of total nucleic acids per reaction and were incubated in the 7900 Fast System SDS 2.3 software.

TABLE O
List of miRNAs

| miR Name | Alternate Name | Sequence |
|---|---|---|
| miR-375 | miR-375 | uuuguucguritggcucgcguga |
| miR-7 | miR-7-5p | uggaagacuagugauuuuguugu |
| miR-15a | miR-15a-5p | uagcagcacauaaugguuugug |
| miR-19b | miR-19b-3p | ugugcaaauccaugcaaaacuga |
| miR-21 | miR-21-5p | uagcuuaucagacugauguuga |
| miR-29b | miR-29b-3p | uagcaccauuugaaaucaguguu |
| miR-29b-1* | miR-29b-1-5p | gcugguuucauauggugguuuaga |
| miR-31 | miR-31-5p | aggcaagaugcuggcauagcu |
| miR-34a | miR-34a-5p | uggcagugucuuagcugguugu |
| miR-96 | miR-96-5p | uuuggcacuagcacauuuuugcu |
| miR-138-1* | miR-138-1-3p | gcuacuucacaacaccagggcc |
| miR-139-5p | miR-139-5p | ucuacagugcacgugucuCcag |
| miR-141 | miR-141-3p | uaacacugucugguaaagaugg |
| miR-146b-3p | miR446b-3p | ugcccuguggacucaguucugg |
| miR-146b-5p | miR-1466-5p | ugagaacugaauuccauaggcu |
| miR-151-3p | miR-151a-3p | cuagacugaagcuccuugagg |
| miR-155 | miR-155-5p | uuaaugcuaauc,gugauaggggu |
| miR-1318-2* | miR-181a-2-3p | accacugaccguugacuguacc |
| miR-197 | miR-197-3p | uucaccaccuucuccacccagc |
| miR-204 | miR-204-5p | uucccuuugucauccuaugccu |
| miR-221 | miR-221-3p | agcuacauugutugcuggguuuc |
| miR-221* | miR-221-5p | accuggcauacaauguagauuu |
| miR-222 | miR-222-3p | agcuacaucuggcuacugggu |
| miR-328 | miR-328 | cuggcccucucugcccuuccgu |
| miR-424 | miR-424-5p | cagcagcaauucauguuuugaa |
| miR-551b | miR-551b-3p | gcgacccauacuugguuucag |

The 41 FFPE samples (Set3), including 21 malignant and 20 benign thyroid neoplasms were analyzed. RT-qPCR results for each replicate of the 26 miRNA assays are summarized in Table Z2. Similarly, the 62 FNA samples (Set3) were also analyzed. RT-qPCR results for each replicate of the 26 miRNA assays are summarized in Table W2.

TABLE Z2
qRT-PCR data obtained using the third set (Set3) of FFPE samples,
corresponding to benign and malignant thyroid neoplasms as
described in Table Z1. Each sample was tested in duplicate. Rep 1
indicates replicate 1 and Rep 2 indicates replicate 2.
The miRNAs indicated in italics were used as putative normalizers.

| Sample | miR- | miR- | miR- | miR- | miR- | miR- |
|---|---|---|---|---|---|---|
| Name | 138-1-3p | 139-5p | 141-3p | 146b-3p | 146b-5p | 151-3p |
| AF184 | 33.37 | 30.93 | 24.00 | 29.92 | 26.11 | 27.86 |
| | 33.90 | 30.53 | 23.90 | 30.01 | 26.24 | 28.18 |
| AF185 | 33.27 | 31.44 | 25.28 | 27.98 | 24.10 | 29.61 |
| | 33.52 | 31.20 | 25.29 | 28.01 | 24.05 | 29.89 |
| AF186 | 33.80 | 31.26 | 25.33 | 27.57 | 23.75 | 28.44 |
| | 32.27 | 31.44 | 25.37 | 27.47 | 23.77 | 28.40 |
| AF187 | 33.49 | 31.59 | 24.62 | 27.48 | 23.08 | 28.20 |
| | 32.98 | 31.16 | 24.58 | 27.39 | 22.98 | 28.32 |
| AF188 | 39.01 | 30.94 | 23.57 | 35.83 | 30.82 | 29.22 |
| | 36.12 | 31.30 | 23.36 | 34.34 | 30.62 | 29.27 |
| AF189 | 32.75 | 29.91 | 24.54 | 26.44 | 22.58 | 28.25 |
| | 32.51 | 30.14 | 24.66 | 26.70 | 22.48 | 28.26 |
| AF190 | 33.09 | 30.58 | 24.38 | 26.81 | 23.25 | 27.97 |
| | 31.99 | 30.93 | 24.22 | 26.91 | 23.20 | 28.29 |
| AF191 | 32.91 | 30.62 | 27.24 | 33.69 | 28.88 | 30.28 |
| | 33.18 | 30.63 | 27.22 | 32.34 | 28.55 | 30.25 |
| AF192 | 32.68 | 31.16 | 26.17 | 31.94 | 28.33 | 30.29 |
| | 32.91 | 30.72 | 25.96 | 32.36 | 28.46 | 30.52 |
| AF193 | 34.53 | 31.48 | 28.29 | 32.79 | 28.65 | 30.86 |
| | 36.29 | 31.80 | 27.86 | 32.17 | 28.63 | 30.72 |
| AF194 | 32.42 | 30.51 | 26.39 | 31.97 | 27.97 | 30.74 |
| | 33.37 | 31.25 | 26.43 | 32.54 | 28.20 | 30.36 |
| AF195 | 28.71 | 26.77 | 23.23 | 33.61 | 30.63 | 27.24 |
| | 28.73 | 26.86 | 23.65 | 33.23 | 30.70 | 27.59 |
| AF196 | 30.88 | 28.69 | 24.77 | 33.94 | 30.13 | 28.47 |
| | 31.59 | 28.72 | 24.68 | 34.95 | 29.56 | 28.50 |
| AF197 | 33.39 | 30.32 | 26.60 | 35.33 | 30.72 | 30.44 |
| | 33.01 | 30.22 | 26.32 | 34.39 | 30.59 | 30.25 |
| AF198 | 29.09 | 29.04 | 22.49 | 32.40 | 28.49 | 27.54 |
| | 29.38 | 28.83 | 22.56 | 32.96 | 28.75 | 27.45 |
| AF199 | 30.82 | 30.90 | 31.36 | 30.55 | 26.40 | 28.41 |
| | 30.62 | 31.20 | 31.79 | 30.37 | 26.31 | 28.46 |
| AF200 | 30.88 | 29.76 | 22.36 | 24.84 | 20.17 | 26.64 |
| | 30.76 | 29.83 | 22.32 | 24.79 | 20.24 | 26.76 |
| AF201 | 31.70 | 28.68 | 23.86 | 26.56 | 22.32 | 27.52 |
| | 31.01 | 28.61 | 23.78 | 26.46 | 22.17 | 27.34 |
| AF202 | 36.10 | 30.76 | 24.65 | 26.52 | 22.79 | 28.19 |
| | 35.12 | 30.98 | 24.26 | 26.55 | 22.40 | 28.21 |
| AF203 | 30.47 | 28.22 | 22.81 | 31.14 | 26.47 | 27.41 |
| | 31.25 | 28.71 | 23.10 | 31.27 | 27.10 | 27.62 |
| AF204 | 30.01 | 29.26 | 24.31 | 31.63 | 28.00 | 27.92 |
| | 30.33 | 29.23 | 24.26 | 32.10 | 27.92 | 28.15 |
| AF205 | 30.77 | 29.19 | 25.19 | 28.90 | 25.41 | 28.25 |
| | 31.44 | 28.94 | 25.13 | 28.78 | 25.51 | 27.97 |
| AF206 | 31.30 | 30.07 | 24.25 | 29.17 | 25.36 | 28.32 |
| | 32.07 | 29.92 | 24.39 | 29.66 | 25.52 | 28.82 |
| AF207 | 30.14 | 28.94 | 25.97 | 30.48 | 27.58 | 28.28 |
| | 30.67 | 28.87 | 25.82 | 30.74 | 27.53 | 28.14 |
| AF208 | 31.13 | 29.38 | 23.38 | 32.60 | 29.68 | 27.80 |
| | 30.95 | 29.27 | 23.41 | 32.28 | 29.76 | 28.23 |
| AF209 | 31.20 | 29.18 | 25.89 | 29.14 | 25.91 | 28.38 |
| | 31.10 | 29.36 | 26.22 | 28.99 | 26.58 | 28.81 |
| AF210 | 30.89 | 28.94 | 24.63 | 27.38 | 23.82 | 27.74 |
| | 31.16 | 28.82 | 24.61 | 27.47 | 24.06 | 27.69 |
| AF211 | 32.43 | 30.43 | 23.53 | 27.17 | 22.96 | 28.32 |
| | 32.76 | 31.17 | 23.67 | 27.32 | 23.20 | 28.56 |
| AF212 | 33.08 | 30.23 | 25.45 | 31.21 | 27.42 | 29.47 |
| | 33.20 | 30.02 | 25.23 | 31.18 | 26.96 | 28.72 |
| AF213 | 30.79 | 30.47 | 23.74 | 26.66 | 22.50 | 27.74 |
| | 31.44 | 29.89 | 23.71 | 26.53 | 22.42 | 27.67 |
| AF214 | 31.26 | 29.67 | 23.79 | 26.62 | 22.77 | 27.77 |
| | 30.93 | 29.96 | 23.88 | 26.60 | 22.64 | 28.10 |
| AF215 | 29.35 | 26.96 | 21.73 | 23.70 | 19.31 | 25.75 |
| | 29.51 | 26.99 | 21.79 | 23.87 | 19.43 | 25.95 |
| AF216 | 28.67 | 30.68 | 22.91 | 33.21 | 29.41 | 27.53 |
| | 28.69 | 29.89 | 23.00 | 33.99 | 29.29 | 27.64 |
| AF217 | 28.98 | 29.86 | 24.01 | 34.17 | 30.10 | 27.93 |
| | 29.69 | 29.69 | 23.99 | 33.10 | 29.81 | 28.21 |
| AF218 | 28.90 | 28.65 | 23.47 | 33.47 | 29.63 | 27.55 |
| | 29.67 | 28.76 | 23.41 | 34.56 | 29.77 | 27.61 |
| AF219 | 29.10 | 28.85 | 23.27 | 34.23 | 29.60 | 27.51 |
| | 29.68 | 28.99 | 23.54 | 33.18 | 29.86 | 27.92 |
| AF220 | 29.26 | 30.45 | 23.23 | 34.49 | 30.15 | 27.85 |
| | 28.76 | 30.76 | 23.19 | 34.39 | 30.45 | 27.85 |
| AF221 | 30.47 | 28.28 | 23.29 | 34.45 | 29.18 | 27.98 |
| | 30.44 | 28.32 | 23.47 | 37.39 | 29.59 | 28.23 |
| AF222 | 30.31 | 28.47 | 23.38 | 33.44 | 30.44 | 27.88 |
| | 30.36 | 28.27 | 23.32 | 35.86 | 30.45 | 27.91 |
| AF223 | 32.58 | 30.68 | 25.93 | 31.70 | 28.00 | 29.28 |
| | 33.20 | 30.83 | 26.41 | 33.27 | 28.29 | 29.54 |
| AF224 | 34.11 | 31.25 | 28.21 | 31.88 | 29.11 | 31.03 |
| | 34.37 | 31.79 | 28.51 | 33.61 | 29.54 | 31.33 |
| Sample | miR- | miR- | miR- | miR- | miR- | miR- |
| Name | 155 | 15a-5p | 181a-2-3p | 197-3p | 19b | 204-5p |
| AF184 | 30.49 | 23.80 | 31.18 | 30.06 | 24.57 | 27.98 |
| | 30.30 | 23.87 | 31.53 | 29.55 | 24.64 | 28.01 |
| AF185 | 30.29 | 24.70 | 31.49 | 31.32 | 25.96 | 32.72 |
| | 30.02 | 24.82 | 31.92 | 30.67 | 26.11 | 32.53 |
| AF186 | 28.09 | 24.90 | 29.75 | 29.87 | 25.31 | 30.84 |
| | 27.90 | 25.10 | 29.92 | 29.73 | 25.40 | 30.38 |
| AF187 | 28.52 | 24.85 | 30.78 | 28.89 | 25.31 | 32.65 |
| | 28.15 | 24.58 | 30.97 | 28.81 | 25.33 | 35.06 |
| AF188 | 31.26 | 24.33 | 32.39 | 28.93 | 24.54 | 31.93 |
| | 31.32 | 24.34 | 32.73 | 29.31 | 24.50 | 32.43 |
| AF189 | 27.35 | 23.92 | 30.76 | 28.49 | 24.73 | 30.45 |
| | 27.25 | 24.16 | 31.46 | 29.12 | 24.77 | 30.62 |
| AF190 | 28.87 | 24.57 | 29.79 | 29.43 | 24.52 | 33.68 |
| | 29.28 | 25.14 | 29.92 | 29.51 | 24.78 | 33.22 |
| AF191 | 26.38 | 25.43 | 33.40 | 29.84 | 25.40 | 31.57 |
| | 26.18 | 25.48 | 32.49 | 29.90 | 25.40 | 31.36 |
| AF192 | 27.34 | 25.90 | 34.32 | 30.47 | 26.03 | 30.83 |
| | 27.15 | 25.72 | 33.53 | 30.09 | 25.99 | 31.16 |
| AF193 | 26.85 | 26.24 | 32.67 | 29.75 | 26.41 | 32.24 |
| | 26.94 | 26.18 | 34.40 | 30.47 | 26.19 | 32.36 |
| AF194 | 26.36 | 25.18 | 31.50 | 29.64 | 25.81 | 31.10 |
| | 26.69 | 25.49 | 33.61 | 30.19 | 25.95 | 31.18 |
| AF195 | 27.25 | 23.91 | 28.32 | 28.29 | 24.06 | 32.69 |
| | 27.71 | 24.17 | 28.35 | 28.64 | 24.20 | 32.50 |
| AF196 | 27.62 | 24.74 | 30.58 | 29.48 | 25.14 | 27.34 |
| | 27.60 | 24.57 | 30.90 | 29.57 | 25.00 | 27.32 |
| AF197 | 28.49 | 26.10 | 32.68 | 30.99 | 26.54 | 29.74 |
| | 28.50 | 26.17 | 33.40 | 30.73 | 26.65 | 30.10 |
| AF198 | 26.95 | 23.36 | 30.31 | 29.17 | 23.61 | 26.67 |
| | 26.97 | 23.52 | 29.78 | 29.15 | 23.65 | 26.64 |
| AF199 | 23.59 | 23.65 | 31.01 | 27.79 | 24.53 | 33.57 |
| | 23.56 | 23.70 | 30.90 | 27.75 | 24.59 | 32.89 |
| AF200 | 27.84 | 22.90 | 28.14 | 28.41 | 22.71 | 30.76 |
| | 28.00 | 22.88 | 28.27 | 28.21 | 22.71 | 30.87 |
| AF201 | 27.71 | 23.41 | 29.37 | 28.31 | 24.17 | 28.53 |
| | 27.69 | 23.21 | 29.32 | 28.65 | 24.09 | 28.55 |
| AF202 | 26.90 | 23.98 | 29.75 | 29.25 | 24.39 | 32.07 |
| | 26.81 | 23.93 | 29.93 | 28.85 | 24.33 | 31.33 |
| AF203 | 28.63 | 23.21 | 28.80 | 28.91 | 23.79 | 27.86 |
| | 28.61 | 23.63 | 28.87 | 28.88 | 24.29 | 28.35 |
| AF204 | 25.70 | 23.79 | 30.75 | 29.21 | 24.96 | 27.28 |
| | 25.71 | 23.78 | 30.53 | 29.67 | 24.95 | 27.67 |
| AF205 | 28.98 | 25.46 | 29.46 | 28.94 | 25.24 | 28.08 |
| | 29.12 | 25.53 | 29.53 | 29.26 | 25.35 | 28.28 |
| AF206 | 30.14 | 24.73 | 28.99 | 29.59 | 24.96 | 30.38 |
| | 30.34 | 25.15 | 29.43 | 29.68 | 25.31 | 30.46 |
| AF207 | 25.98 | 24.73 | 30.59 | 28.55 | 24.90 | 26.78 |
| | 25.90 | 24.85 | 30.66 | 28.57 | 25.00 | 26.88 |
| AF208 | 29.40 | 24.39 | 28.84 | 29.56 | 24.54 | 27.13 |
| | 29.27 | 24.44 | 28.68 | 29.58 | 24.62 | 27.23 |
| AF209 | 29.26 | 26.36 | 30.68 | 28.50 | 25.41 | 27.78 |
| | 29.35 | 26.59 | 31.00 | 28.63 | 25.72 | 27.63 |
| AF210 | 28.52 | 24.47 | 28.37 | 28.90 | 24.46 | 28.67 |
| | 28.57 | 24.48 | 28.98 | 28.98 | 24.49 | 28.78 |
| AF211 | 29.24 | 24.27 | 29.38 | 29.69 | 23.83 | 31.65 |
| | 29.27 | 24.67 | 29.31 | 29.63 | 24.13 | 32.17 |
| AF212 | 25.20 | 24.23 | 31.84 | 28.79 | 24.51 | 30.55 |
| | 24.87 | 24.09 | 32.03 | 28.58 | 24.30 | 30.37 |
| AF213 | 29.38 | 24.49 | 23.16 | 28.68 | 24.00 | 32.75 |
| | 29.47 | 24.53 | 28.13 | 28.83 | 23.91 | 33.30 |
| AF214 | 27.70 | 23.59 | 29.30 | 29.17 | 24.42 | 30.72 |
| | 27.84 | 23.69 | 29.35 | 29.24 | 24.51 | 30.74 |
| AF215 | 26.59 | 21.84 | 26.78 | 26.63 | 21.87 | 27.03 |
| | 26.48 | 21.98 | 26.87 | 26.96 | 21.99 | 27.19 |
| AF216 | 28.52 | 24.01 | 28.18 | 28.97 | 23.33 | 26.55 |
| | 28.30 | 23.91 | 28.18 | 28.90 | 23.57 | 26.25 |
| AF217 | 29.24 | 24.22 | 29.37 | 28.79 | 23.99 | 26.72 |
| | 28.84 | 24.35 | 29.43 | 28.71 | 24.02 | 26.72 |
| AF218 | 28.33 | 23.62 | 29.52 | 28.83 | 23.59 | 28.28 |
| | 28.51 | 23.73 | 30.22 | 28.50 | 23.72 | 27.93 |
| AF219 | 28.61 | 24.17 | 29.34 | 28.81 | 23.67 | 26.94 |
| | 28.33 | 24.53 | 29.75 | 29.36 | 24.13 | 27.20 |
| AF220 | 29.91 | 24.79 | 29.83 | 29.81 | 23.95 | 26.58 |
| | 29.56 | 24.79 | 30.10 | 29.65 | 23.96 | 26.41 |
| AF221 | 27.74 | 24.30 | 30.23 | 28.72 | 24.23 | 28.84 |
| | 27.79 | 24.37 | 30.39 | 28.81 | 24.31 | 28.78 |
| AF222 | 29.24 | 24.61 | 30.72 | 29.19 | 24.28 | 27.32 |
| | 29.15 | 24.62 | 30.57 | 29.15 | 24.46 | 27.31 |
| AF223 | 25.48 | 24.55 | 31.88 | 29.54 | 24.54 | 29.73 |
| | 25.72 | 24.72 | 32.30 | 29.53 | 24.82 | 29.59 |
| AF224 | 27.60 | 26.28 | 32.51 | 30.28 | 26.66 | 31.67 |
| | 27.66 | 26.78 | 32.53 | 30.10 | 26.78 | 31.96 |
| Sample | miR- | miR- | miR- | miR- | miR- | miR- |
| Name | 21-5p | 221-3p | 221-5p | 222-3p | 29b-1* | 29b-3p |
| AF184 | 22.60 | 23.61 | 30.03 | 22.63 | 30.74 | 25.78 |
| | 22.81 | 23.84 | 29.41 | 22.80 | 29.97 | 25.75 |
| AF185 | 23.18 | 24.52 | 30.32 | 22.93 | 30.99 | 27.31 |
| | 22.93 | 24.53 | 31.22 | 22.88 | 31.24 | 27.20 |
| AF186 | 21.60 | 24.18 | 30.28 | 22.85 | 31.39 | 26.77 |
| | 21.73 | 24.31 | 30.43 | 22.88 | 31.90 | 26.87 |
| AF187 | 20.58 | 24.16 | 30.49 | 22.18 | 30.97 | 26.02 |
| | 20.58 | 24.11 | 29.40 | 22.14 | 29.80 | 25.77 |
| AF188 | 21.77 | 28.96 | 33.38 | 27.47 | 30.49 | 24.58 |
| | 21.80 | 29.34 | 32.95 | 26.56 | 30.01 | 25.07 |
| AF189 | 20.22 | 23.71 | 29.84 | 22.46 | 29.54 | 25.63 |
| | 20.61 | 23.65 | 30.07 | 22.46 | 30.25 | 25.72 |
| AF190 | 20.77 | 23.92 | 31.25 | 22.45 | 30.46 | 27.02 |
| | 21.06 | 23.82 | 30.43 | 22.60 | 30.76 | 26.70 |
| AF191 | 22.86 | 28.11 | 33.88 | 25.99 | 31.65 | 27.45 |
| | 22.73 | 28.11 | 33.34 | 26.01 | 31.54 | 27.22 |
| AF192 | 22.07 | 28.45 | 34.16 | 26.35 | 31.20 | 27.33 |
| | 22.16 | 28.39 | 33.69 | 26.26 | 31.36 | 27.20 |
| AF193 | 23.19 | 28.01 | 33.31 | 26.00 | 32.22 | 28.20 |
| | 23.12 | 27.93 | 33.63 | 26.35 | 31.77 | 28.13 |
| AF194 | 21.69 | 26.90 | 32.77 | 25.79 | 31.73 | 27.16 |
| | 21.82 | 27.12 | 34.87 | 25.98 | 31.79 | 27.34 |
| AF195 | 21.75 | 25.80 | 33.79 | 25.74 | 31.65 | 26.41 |
| | 22.16 | 26.27 | 34.92 | 26.22 | 32.56 | 26.82 |
| AF196 | 22.51 | 28.58 | 33.84 | 27.10 | 30.87 | 27.13 |
| | 22.41 | 28.66 | 34.36 | 27.18 | 30.62 | 26.83 |
| AF197 | 23.73 | 28.91 | 35.09 | 27.33 | 34.11 | 28.33 |
| | 23.76 | 28.89 | 38.69 | 27.57 | 31.81 | 27.88 |
| AF198 | 21.51 | 27.39 | 32.41 | 25.71 | 29.54 | 25.21 |
| | 21.68 | 27.61 | 32.26 | 25.79 | 29.90 | 21.37 |
| AF199 | 19.45 | 24.96 | 32.54 | 25.36 | 31.36 | 27.25 |
| | 19.48 | 25.17 | 32.92 | 25.41 | 31.35 | 27.08 |
| AF200 | 19.20 | 21.92 | 27.07 | 20.51 | 29.42 | 23.69 |
| | 19.23 | 21.96 | 27.14 | 20.54 | 29.41 | 23.59 |
| AF201 | 21.52 | 23.31 | 28.85 | 21.87 | 30.20 | 24.87 |
| | 21.37 | 23.14 | 28.93 | 21.84 | 30.20 | 24.62 |
| AF202 | 20.11 | 24.56 | 32.29 | 22.62 | 31.73 | 25.94 |
| | 20.35 | 24.40 | 30.48 | 22.58 | 30.79 | 25.14 |
| AF203 | 21.98 | 24.13 | 29.89 | 23.18 | 30.08 | 25.20 |
| | 22.25 | 24.53 | 30.06 | 23.48 | 30.49 | 25.63 |
| AF204 | 21.17 | 27.98 | 32.91 | 26.31 | 30.74 | 26.32 |
| | 21.26 | 28.12 | 33.95 | 26.24 | 30.93 | 26.42 |
| AF205 | 23.53 | 24.61 | 30.35 | 23.84 | 30.14 | 26.70 |
| | 23.61 | 24.79 | 30.57 | 24.34 | 30.90 | 26.77 |
| AF206 | 23.62 | 24.92 | 31.05 | 24.00 | 30.83 | 25.83 |
| | 24.10 | 25.34 | 31.27 | 24.22 | 31.49 | 26.14 |
| AF207 | 21.91 | 26.61 | 32.85 | 26.36 | 30.31 | 26.42 |
| | 22.20 | 26.71 | 32.47 | 26.20 | 30.15 | 26.53 |
| AF208 | 22.60 | 22.76 | 29.38 | 22.45 | 31.78 | 25.42 |
| | 22.67 | 22.76 | 29.68 | 22.36 | 30.76 | 25.62 |
| AF209 | 23.63 | 25.34 | 32.92 | 24.70 | 30.57 | 27.58 |
| | 23.97 | 25.88 | 32.56 | 24.98 | 30.44 | 27.57 |
| AF210 | 20.92 | 23.92 | 30.30 | 22.71 | 30.37 | 26.14 |
| | 20.90 | 23.99 | 30.25 | 22.82 | 30.22 | 26.37 |
| AF211 | 21.15 | 24.56 | 29.81 | 21.69 | 29.86 | 25.63 |
| | 21.36 | 24.88 | 30.26 | 21.83 | 30.15 | 26.13 |
| AF212 | 21.14 | 27.50 | 33.12 | 25.46 | 31.51 | 26.52 |
| | 20.88 | 27.01 | 32.31 | 25.21 | 30.91 | 25.97 |
| AF213 | 21.01 | 22.92 | 29.45 | 21.94 | 30.33 | 25.51 |
| | 20.99 | 22.77 | 29.13 | 21.88 | 29.96 | 25.31 |
| AF214 | 20.96 | 23.58 | 29.12 | 22.16 | 30.14 | 25.14 |
| | 21.32 | 23.77 | 29.21 | 22.18 | 30.50 | 25.30 |
| AF215 | 18.89 | 22.86 | 28.48 | 21.59 | 27.98 | 22.83 |
| | 19.00 | 23.13 | 28.22 | 21.68 | 27.99 | 22.96 |
| AF216 | 21.58 | 25.49 | 31.04 | 24.08 | 29.82 | 25.54 |
| | 21.62 | 25.69 | 29.91 | 23.67 | 30.12 | 25.69 |
| AF217 | 22.74 | 24.74 | 30.78 | 23.88 | 29.65 | 25.87 |
| | 22.73 | 24.69 | 29.97 | 24.02 | 29.82 | 25.86 |
| AF218 | 22.38 | 26.60 | 32.41 | 24.55 | 29.79 | 25.25 |
| | 22.30 | 26.50 | 31.12 | 24.60 | 29.39 | 24.86 |
| AF219 | 22.17 | 26.73 | 30.69 | 24.42 | 29.77 | 25.18 |
| | 22.43 | 26.63 | 31.78 | 24.68 | 29.77 | 25.76 |
| AF220 | 22.87 | 26.61 | 30.91 | 24.65 | 29.83 | 24.97 |
| | 22.85 | 26.71 | 30.61 | 24.68 | 29.84 | 25.13 |
| AF221 | 22.25 | 26.33 | 31.66 | 25.37 | 29.77 | 25.28 |
| | 22.48 | 26.69 | 31.41 | 25.35 | 29.87 | 25.56 |
| AF222 | 22.75 | 27.65 | 32.54 | 26.46 | 30.43 | 25.57 |
| | 22.66 | 27.35 | 32.20 | 26.61 | 29.99 | 25.68 |
| AF223 | 21.70 | 27.76 | 33.53 | 25.55 | 31.36 | 26.53 |
| | 21.83 | 28.43 | 33.72 | 25.76 | 31.56 | 26.87 |
| AF224 | 22.84 | 28.64 | 35.25 | 27.09 | 32.26 | 27.79 |
| | 23.23 | 29.15 | 34.07 | 27.24 | 31.70 | 28.08 |
| Sample | miR- | miR- | miR- | miR- | miR- | miR- |
| Name | 31-5p | 328 | 34a | 375 | 424-5p | 551b-3p |
| AF184 | 23.91 | 29.47 | 25.09 | 30.85 | 27.10 | 26.12 |
| | 23.63 | 29.29 | 24.90 | 30.88 | 27.13 | 25.93 |
| AF185 | 29.26 | 30.54 | 26.15 | 29.95 | 26.54 | 31.18 |
| | 29.23 | 30.63 | 26.34 | 29.40 | 26.88 | 32.35 |
| AF186 | 24.88 | 28.72 | 25.71 | 28.23 | 25.68 | 27.41 |
| | 25.12 | 28.84 | 25.98 | 28.39 | 25.77 | 27.80 |
| AF187 | 25.53 | 28.63 | 24.86 | 28.47 | 26.54 | 28.41 |
| | 25.42 | 28.75 | 24.81 | 28.63 | 26.40 | 28.24 |
| AF188 | 30.45 | 29.42 | 24.25 | 31.51 | 28.21 | 40.29 |
| | 30.15 | 29.48 | 24.23 | 31.61 | 28.42 | 38.28 |
| AF189 | 26.67 | 28.29 | 24.62 | 28.13 | 25.11 | 27.40 |
| | 27.16 | 28.22 | 24.99 | 28.22 | 25.33 | 27.96 |
| AF190 | 27.47 | 28.49 | 25.15 | 31.18 | 25.54 | 29.71 |
| | 27.49 | 28.52 | 25.50 | 31.58 | 25.76 | 28.72 |
| AF191 | 29.31 | 30.85 | 28.09 | 31.89 | 27.75 | 32.70 |
| | 29.12 | 31.45 | 27.65 | 33.21 | 27.82 | 33.22 |
| AF192 | 27.64 | 31.44 | 27.44 | 35.55 | 26.81 | 34.94 |
| | 27.57 | 31.15 | 27.29 | 35.47 | 26.72 | 33.62 |
| AF193 | 28.91 | 31.12 | 28.24 | 34.39 | 27.85 | 34.74 |
| | 28.79 | 30.57 | 27.87 | 33.85 | 27.69 | 32.35 |
| AF194 | 27.90 | 30.29 | 26.71 | 41.82 | 26.87 | 37.87 |
| | 28.36 | 31.69 | 27.18 | 41.91 | 27.17 | 32.58 |
| AF195 | 30.20 | 29.30 | 26.31 | 33.39 | 24.93 | 30.17 |
| | 30.39 | 29.86 | 26.65 | 35.30 | 25.44 | 31.09 |
| AF196 | 29.15 | 29.54 | 26.80 | 34.66 | 25.24 | 33.52 |
| | 28.80 | 29.59 | 26.45 | 34.58 | 24.96 | 32.93 |
| AF197 | 30.78 | 31.49 | 27.41 | 34.28 | 26.94 | 35.53 |
| | 30.88 | 31.90 | 27.64 | 34.49 | 26.84 | 32.98 |
| AF198 | 29.50 | 28.61 | 24.47 | 31.80 | 24.42 | 32.76 |
| | 29.55 | 28.48 | 24.56 | 31.52 | 24.47 | 32.54 |
| AF199 | 26.74 | 29.64 | 25.54 | 43.36 | 24.86 | 32.38 |
| | 26.83 | 30.19 | 25.44 | 41.80 | 24.95 | 32.30 |
| AF200 | 22.70 | 28.46 | 23.45 | 26.27 | 21.64 | 24.91 |
| | 22.65 | 28.45 | 23.39 | 26.04 | 21.74 | 24.95 |
| AF201 | 23.72 | 28.16 | 24.91 | 29.25 | 22.74 | 27.49 |
| | 23.49 | 28.18 | 24.66 | 28.98 | 22.68 | 27.43 |
| AF202 | 24.20 | 28.90 | 24.82 | 27.94 | 25.23 | 27.17 |
| | 23.93 | 29.15 | 25.00 | 27.75 | 25.37 | 27.09 |
| AF203 | 30.73 | 28.12 | 25.46 | 26.69 | 22.59 | 34.30 |
| | 30.58 | 28.18 | 25.80 | 27.00 | 23.09 | 33.19 |
| AF204 | 29.67 | 29.55 | 26.16 | 37.80 | 24.71 | 32.37 |
| | 29.50 | 29.95 | 26.22 | 34.75 | 24.92 | 36.36 |
| AF205 | 26.94 | 28.33 | 26.77 | 27.28 | 26.78 | 29.62 |
| | 26.90 | 27.78 | 26.73 | 27.47 | 26.12 | 29.86 |
| AF206 | 31.02 | 45.00 | 26.24 | 27.66 | 27.60 | 31.92 |
| | 30.62 | 28.67 | 26.61 | 27.76 | 27.90 | 32.69 |
| AF207 | 27.55 | 28.51 | 25.95 | 33.88 | 26.35 | 32.53 |
| | 27.51 | 28.61 | 25.93 | 32.82 | 26.46 | 32.41 |
| AF208 | 25.52 | 29.53 | 25.41 | 31.66 | 24.26 | 24.62 |
| | 25.86 | 29.39 | 25.60 | 31.50 | 24.45 | 24.65 |
| AF209 | 27.74 | 27.49 | 26.42 | 29.38 | 26.41 | 30.26 |
| | 27.51 | 27.55 | 27.00 | 29.56 | 26.78 | 30.58 |
| AF210 | 25.28 | 28.46 | 25.24 | 28.43 | 24.34 | 27.26 |
| | 25.24 | 28.36 | 25.06 | 28.18 | 24.49 | 27.52 |
| AF211 | 26.79 | 29.11 | 25.44 | 27.26 | 27.70 | 27.14 |
| | 26.75 | 29.31 | 25.67 | 27.64 | 28.38 | 27.45 |
| AF212 | 27.72 | 29.98 | 26.74 | 33.51 | 25.96 | 32.37 |
| | 27.34 | 29.40 | 26.54 | 31.52 | 25.78 | 30.82 |
| AF213 | 24.95 | 28.27 | 24.65 | 26.66 | 24.14 | 25.87 |
| | 25.07 | 28.16 | 24.70 | 26.67 | 24.15 | 26.09 |
| AF214 | 23.40 | 28.55 | 24.45 | 24.89 | 26.70 | 26.72 |
| | 23.38 | 28.44 | 24.77 | 26.34 | 26.85 | 26.82 |
| AF215 | 22.11 | 26.20 | 23.71 | 24.27 | 20.96 | 24.38 |
| | 22.26 | 26.28 | 23.86 | 24.39 | 21.01 | 24.53 |
| AF216 | 29.09 | 27.94 | 24.95 | 30.97 | 23.41 | 31.20 |
| | 29.26 | 27.80 | 24.90 | 32.34 | 23.45 | 31.81 |
| AF217 | 29.74 | 28.14 | 25.09 | 32.87 | 25.36 | 30.03 |
| | 29.89 | 28.17 | 24.95 | 31.86 | 25.24 | 29.78 |
| AF218 | 28.24 | 28.60 | 25.28 | 30.85 | 23.43 | 30.96 |
| | 27.87 | 28.50 | 25.39 | 30.84 | 23 57 | 30.55 |
| AF219 | 30.24 | 27.95 | 24.61 | 31.50 | 24.53 | 31.38 |
| | 30.24 | 28.14 | 24.98 | 32.68 | 24.78 | 33.54 |
| AF220 | 28.08 | 28.66 | 25.22 | 34.30 | 23.62 | 32.39 |
| | 27.87 | 28.80 | 25.36 | 34.63 | 24.61 | 31.15 |
| AF221 | 27.84 | 28.44 | 25.79 | 29.48 | 23.79 | 30.95 |
| | 27.89 | 28.36 | 25.99 | 29.82 | 23.99 | 32.01 |
| AF222 | 30.34 | 28.45 | 25.31 | 32.21 | 24.86 | 32.15 |
| | 30.55 | 28.51 | 25.12 | 33.83 | 24.64 | 33.82 |
| AF223 | 27.94 | 30.49 | 27.22 | 32.20 | 26.26 | 32.45 |
| | 28.39 | 30.61 | 27.35 | 32.49 | 26.47 | 34.59 |
| AF224 | 29.34 | 30.72 | 27.78 | 35.15 | 28.13 | 34.65 |
| | 29.27 | 31.12 | 27.83 | 35.55 | 28.13 | 34.75 |

| Sample | miR- | miR- |
|---|---|---|
| Name | 7-5p | 96-5p |
| AF184 | 32.38 | 28.58 |
| | 31.25 | 28.68 |
| AF185 | 35.62 | 32.95 |
| | 36.76 | 32.45 |
| AF186 | 33.26 | 31.61 |
| | 33.76 | 31.99 |
| AF187 | 32.38 | 31.18 |
| | 32.15 | 30.62 |
| AF188 | 32.73 | 28.32 |
| | 32.14 | 28.62 |
| AF189 | 30.31 | 30.76 |
| | 30.60 | 31.78 |
| AF190 | 33.43 | 30.53 |
| | 33.47 | 30.81 |
| AF191 | 32.65 | 33.97 |
| | 32.11 | 35.30 |
| AF192 | 33.98 | 32.93 |
| | 33.54 | 32.53 |
| AF193 | 33.82 | 34.25 |
| | 33.85 | 34.15 |
| AF194 | 32.61 | 32.91 |
| | 32.93 | 32.66 |
| AF195 | 31.89 | 35.36 |
| | 32.40 | 35.80 |
| AF196 | 27.37 | 30.67 |
| | 27.17 | 30.22 |
| AF197 | 28.29 | 31.70 |
| | 27.90 | 32.02 |
| AF198 | 27.71 | 29.48 |
| | 28.17 | 29.39 |
| AF199 | 30.27 | 32.55 |
| | 29.74 | 32.64 |
| AF200 | 31.66 | 29.82 |
| | 32.09 | 29.87 |
| AF201 | 28.55 | 30.13 |
| | 28.21 | 29.97 |
| AF202 | 33.47 | 31.66 |
| | 31.50 | 31.27 |
| AF203 | 27.77 | 29.53 |
| | 28.47 | 29.93 |
| AF204 | 28.84 | 29.99 |
| | 28.56 | 30.01 |
| AF205 | 28.61 | 32.34 |
| | 28.66 | 32.19 |
| AF206 | 29.65 | 31.17 |
| | 30.18 | 31.84 |
| AF207 | 30.48 | 31.44 |
| | 30.57 | 31.88 |
| AF208 | 31.18 | 30.25 |
| | 31.12 | 30.58 |
| AF209 | 29.73 | 33.16 |
| | 30.08 | 33.73 |
| AF210 | 27.92 | 31.79 |
| | 27.60 | 31.19 |
| AF211 | 33.23 | 32.44 |
| | 34.20 | 32.56 |
| AF212 | 31.46 | 31.87 |
| | 31.50 | 31.54 |
| AF213 | 31.84 | 31.78 |
| | 31.80 | 31.79 |
| AF214 | 29.78 | 30.45 |
| | 30.72 | 30.76 |
| AF215 | 27.81 | 28.67 |
| | 27.76 | 28.84 |
| AF216 | 25.33 | 29.90 |
| | 24.88 | 29.76 |
| AF217 | 28.41 | 29.89 |
| | 28.37 | 30.19 |
| AF218 | 24.78 | 30.60 |
| | 24.35 | 30.88 |
| AF219 | 25.72 | 30.21 |
| | 26.14 | 30.75 |
| AF220 | 25.17 | 30.69 |
| | 24.85 | 30.90 |
| AF221 | 28.00 | 29.65 |
| | 28.25 | 30.03 |
| AF222 | 28.46 | 30.17 |
| | 28.26 | 29.69 |
| AF223 | 31.77 | 32.53 |
| | 32.44 | 35.27 |
| AF224 | 33.52 | 33.20 |
| | 34.45 | 32.84 |

TABLE W2

| | miR- | | | |
|---|---|---|---|---|
| Sample ID | 138-1-3p | miR-139-5p | miR-141-3p | miR-146b-3p |
| FNA-T1 | 29.686714 | 27.247915 | 23.298391 | 23.720663 |
| FNA-T1 | 29.66181 | 27.374592 | 23.236439 | 23.762691 |
| FNA-T10 | 33.610046 | 32.17644 | 27.20892 | 26.287708 |
| FNA-T10 | 32.77893 | 31.632158 | 27.216581 | 26.069279 |
| FNA-T11 | 34.85763 | 34.542107 | 28.939924 | 28.889807 |
| FNA-T11 | 34.45797 | 35.284443 | 29.184425 | 29.125067 |
| FNA-T12 | 34.113934 | 32.123882 | 25.513596 | 27.645618 |
| FNA-T12 | 33.71167 | 33.661522 | 25.607426 | 27.720995 |
| FNA-T13 | 35.781925 | 31.772648 | 24.466602 | 25.980343 |
| FNA-T13 | 33.83142 | 31.801874 | 24.428434 | 25.918755 |
| FNA-T14 | 31.794363 | 30.458538 | 25.926065 | 25.942867 |
| FNA-T14 | 32.492924 | 29.800528 | 26.113035 | 25.78877 |
| FNA-T15 | 31.875889 | 28.51506 | 27.280334 | 30.194223 |
| FNA-T15 | 31.78392 | 28.689842 | 27.146084 | 30.138336 |
| FNA-T16 | 32.932526 | 31.41135 | 27.525618 | 27.007025 |
| FNA-T16 | 33.03669 | 33.427067 | 27.685963 | 27.152893 |
| FNA-T17 | 30.957563 | 29.474812 | 23.295797 | 25.131533 |
| FNA-T17 | 31.223692 | 29.525341 | 23.461391 | 25.27709 |
| FNA-T18 | 32.8673 | 31.442698 | 27.58398 | 26.296366 |
| FNA-T18 | 33.00814 | 32.19118 | 27.509956 | 26.396751 |
| FNA-T19 | 31.111235 | 29.396866 | 24.659824 | 23.909304 |
| FNA-T19 | 31.434946 | 29.203545 | 24.664917 | 23.905048 |
| FNA-T2 | 31.980186 | 30.384403 | 27.85435 | 31.246332 |
| FNA-T2 | 32.488434 | 30.61042 | 28.185389 | 30.63359 |
| FNA-T20 | 30.106356 | 28.865314 | 24.599226 | 32.45335 |
| FNA-T20 | 30.381126 | 28.741804 | 24.478662 | 32.29998 |
| FNA-T21 | 30.102598 | 28.16299 | 24.108892 | 25.423674 |
| FNA-T21 | 30.244219 | 28.410114 | 23.934332 | 25.590101 |
| FNA-T22 | 31.478653 | 32.62077 | 26.917446 | 27.792574 |
| FNA-T22 | 31.91722 | 32.284798 | 26.694897 | 27.384892 |
| FNA-T23 | 30.931576 | 29.706663 | 24.101965 | 27.32051 |
| FNA-T23 | 30.792831 | 30.137056 | 23.953304 | 26.976732 |
| FNA-T24 | 31.998234 | 29.29144 | 26.961124 | 33.33903 |
| FNA-T24 | 32.08647 | 29.505066 | 27.351202 | 33.17794 |
| FNA-T25 | 32.426994 | 32.362892 | 28.1807 | 31.197384 |
| FNA-T25 | 32.599224 | 29.722988 | 28.310392 | 31.630665 |
| FNA-T26 | 32.400253 | 30.824299 | 27.49206 | 33.15483 |
| FNA-T26 | 33.65451 | 30.500172 | 27.622587 | 32.093754 |
| FNA-T27 | 32.102573 | 30.848007 | 27.20775 | 30.494226 |
| FNA-T27 | 32.10568 | 30.899002 | 27.281246 | 30.511189 |
| FNA-T28 | 31.981346 | 29.08321 | 25.901917 | 32.184334 |
| FNA-T28 | 32.593918 | 28.773298 | 25.767479 | 32.46334 |
| FNA-T29 | 29.535822 | 29.254395 | 23.848276 | 32.73343 |
| FNA-T29 | 29.635082 | 29.272427 | 24.182951 | 32.792492 |
| FNA-T3 | 32.538258 | 29.912996 | 25.642756 | 32.796402 |
| FNA-T3 | 31.945688 | 29.87382 | 25.800848 | 33.460773 |
| FNA-T30 | 30.541605 | 28.517384 | 24.828289 | 32.427128 |
| FNA-T30 | 30.92708 | 28.607445 | 25.17766 | 34.77237 |
| FNA-T31 | 29.869715 | 31.146616 | 27.003883 | 31.084644 |
| FNA-T31 | 29.721697 | 30.19459 | 26.63451 | 29.971098 |
| FNA-T32 | 37.196377 | 31.862574 | 27.661999 | Undetermined |
| FNA-T32 | 34.52026 | 33.11717 | 27.899796 | 35.803516 |
| FNA-T33 | 28.37062 | 29.169825 | 23.765234 | 31.671144 |
| FNA-T33 | 28.735172 | 29.671164 | 24.0439 | 31.66935 |
| FNA-T34 | 33.445362 | 31.597311 | 26.182804 | 33.784332 |
| FNA-T34 | 32.082203 | 31.9434 | 25.829365 | 33.603394 |
| FNA-T35 | 29.741611 | 29.717335 | 25.482199 | 31.869898 |
| FNA-T35 | 29.681551 | 29.868559 | 25.616343 | 32.157448 |
| FNA-T36 | 33.54916 | 32.003017 | 28.601177 | 33.726288 |
| FNA-T36 | 33.221252 | 32.413284 | 28.297396 | 33.929947 |
| FNA-T37 | 32.722103 | 29.315485 | 25.895226 | 34.312267 |
| FNA-T37 | 31.734703 | 29.285511 | 26.230925 | 33.356644 |
| FNA-T38 | 28.73019 | 26.1435 | 25.165466 | 31.289324 |
| FNA-T38 | 28.965206 | 26.151163 | 25.321548 | 32.214836 |
| FNA-T39 | 31.352875 | 29.358044 | 26.886892 | 31.125448 |
| FNA-T39 | 30.429163 | 29.271496 | 25.186626 | 30.990368 |
| FNA-T4 | 33.19305 | 32.144253 | 28.6505 | 30.507126 |
| FNA-T4 | 34.16581 | 30.25454 | 28.491386 | 30.651333 |
| FNA-T40 | 30.550085 | 30.673561 | 26.165552 | 33.257618 |
| FNA-T40 | 31.080975 | 30.664106 | 26.151747 | 33.51381 |
| FNA-T41 | 30.562462 | 28.952232 | 25.97411 | 31.680597 |
| FNA-T41 | 31.16829 | 28.875553 | 26.17238 | 32.236702 |
| FNA-T42 | 30.624987 | 27.908648 | 24.428516 | 25.571127 |
| FNA-T42 | 30.658573 | 27.445137 | 24.389067 | 25.594055 |
| FNA-T43 | 30.00317 | 27.26614 | 23.334663 | 24.188713 |
| FNA-T43 | 29.850477 | 27.505629 | 23.338024 | 24.257338 |
| FNA-T44 | 30.574074 | 29.157316 | 22.70063 | 23.928938 |
| FNA-T44 | 30.349606 | 28.867634 | 22.586786 | 23.822565 |
| FNA-T45 | 32.90401 | 31.363914 | 28.723019 | 26.445185 |
| FNA-T45 | 33.089054 | 31.256847 | 28.531202 | 26.53614 |
| FNA-T46 | 30.945957 | 28.765219 | 22.778337 | 23.918518 |
| FNA-T46 | 30.871307 | 28.85428 | 22.668093 | 23.98527 |
| FNA-T47 | 30.607052 | 30.209711 | 25.370367 | 27.159512 |
| FNA-T47 | 30.79872 | 30.541962 | 25.366777 | 27.207943 |
| FNA-T48 | 31.33223 | 30.207405 | 25.478212 | 24.181093 |
| FNA-T48 | 31.012167 | 30.255873 | 25.515608 | 24.226221 |
| FNA-T49 | 30.818712 | 28.274742 | 24.313276 | 24.373377 |
| FNA-T49 | 32.194633 | 27.943735 | 24.212631 | 24.296938 |
| FNA-T5 | 32.405037 | 28.555017 | 27.167114 | 29.384792 |
| FNA-T5 | 31.89376 | 28.703918 | 27.190718 | 29.46035 |
| FNA-T50 | 30.527687 | 28.470112 | 24.317799 | 25.720697 |
| FNA-T50 | 30.176064 | 28.373278 | 24.334843 | 25.726728 |
| FNA-T51 | 30.870789 | 28.721737 | 24.761866 | 24.236048 |
| FNA-T51 | 30.63485 | 29.45917 | 24.92758 | 24.526176 |
| FNA-T52 | 29.374151 | 28.440987 | 22.705618 | 26.206436 |
| FNA-T52 | 29.321453 | 28.479843 | 22.820032 | 26.28629 |
| FNA-T53 | 33.202103 | 29.906212 | 27.915926 | 32.233067 |
| FNA-T53 | 31.661749 | 29.735195 | 27.926538 | 31.778103 |
| FNA-T54 | 31.703917 | 29.97529 | 27.210884 | 34.27559 |
| FNA-T54 | 32.103035 | 29.599464 | 27.247477 | 33.553905 |
| FNA-T55 | 31.507004 | 28.198795 | 25.245266 | 31.651506 |
| FNA-T55 | 30.746473 | 27.802738 | 24.952003 | 31.818302 |
| FNA-T56 | 34.315556 | 32.289394 | 25.77568 | 34.346485 |
| FNA-T56 | 34.449806 | 33.10517 | 25.910244 | 35.411453 |
| FNA-T57 | 32.83361 | 30.722061 | 24.821531 | 28.7195 |
| FNA-T57 | 31.372488 | 30.59778 | 24.864733 | 28.526976 |
| FNA-T58 | 31.128708 | 28.178057 | 28.412926 | 33.540627 |
| FNA-T58 | 30.967634 | 28.466677 | 28.631424 | 34.370808 |
| FNA-T59 | 30.877525 | 29.4581 | 25.55062 | 25.727354 |
| FNA-T59 | 30.99779 | 29.486015 | 25.751541 | 25.748922 |
| FNA-T6 | 31.995312 | 31.7296 | 27.498919 | 35.970116 |
| FNA-T6 | 32.391037 | 32.419277 | 27.872248 | 35.623577 |
| FNA-T60 | 34.730118 | 31.15454 | 26.375692 | Undetermined |
| FNA-T60 | 32.970734 | 30.487375 | 26.474897 | 35.450314 |
| FNA-T61 | 31.383772 | 31.811224 | 26.245752 | 32.74382 |
| FNA-T61 | 31.403366 | 31.867811 | 26.596395 | 35.20468 |
| FNA-T62 | 30.78053 | 30.018875 | 25.911264 | 35.736336 |
| FNA-T62 | 30.81902 | 30.542652 | 25.900553 | 33.17349 |
| FNA-T7 | 31.494045 | 29.200693 | 26.626982 | 27.406225 |
| FNA-T7 | 31.402466 | 29.446598 | 26.651577 | 27.489912 |
| FNA-T8 | 32.83336 | 28.213581 | 25.212255 | 25.867922 |
| FNA-T8 | 32.586514 | 28.385561 | 25.362967 | 25.944284 |
| FNA-T9 | 35.19928 | 32.9607 | 30.458836 | 34.483185 |
| FNA-T9 | 33.972744 | 33.5527 | 31.345991 | 34.213585 |
| Sample ID | miR-146b-5p | miR-151-3p | miR-155 | miR-15a-5p |
| FNA-T1 | 20.532497 | 23.720423 | 23.397497 | 17.395332 |
| FNA-T1 | 20.545517 | 23.844328 | 23.497507 | 17.452892 |
| FNA-T10 | 25.24384 | 27.355364 | 28.774397 | 21.807444 |
| FNA-T10 | 24.460548 | 27.264843 | 28.968596 | 21.666267 |
| FNA-T11 | 27.978056 | 28.215204 | 26.409773 | 21.950428 |
| FNA-T11 | 28.236816 | 28.395224 | 26.613869 | 22.267273 |
| FNA-T12 | 25.318787 | 29.48024 | 30.908276 | 24.910538 |
| FNA-T12 | 25.46194 | 29.567608 | 30.785582 | 24.807781 |
| FNA-T13 | 23.385855 | 28.561462 | 26.950827 | 23.2032 |
| FNA-T13 | 23.349695 | 28.480206 | 26.960596 | 23.313564 |
| FNA-T14 | 24.594042 | 28.338284 | 30.142021 | 24.866877 |
| FNA-T14 | 24.406065 | 28.2445 | 29.90064 | 24.846542 |
| FNA-T15 | 28.103964 | 24.106462 | 26.935848 | 18.304169 |
| FNA-T15 | 28.44457 | 23.958496 | 27.020933 | 18.511219 |
| FNA-T16 | 27.736053 | 29.234398 | 33.175297 | 23.255949 |
| FNA-T16 | 26.794262 | 29.27337 | 31.58349 | 23.282183 |
| FNA-T17 | 22.982538 | 26.318102 | 25.135504 | 23.430061 |
| FNA-T17 | 22.894547 | 26.406599 | 25.233057 | 23.517857 |
| FNA-T18 | 25.324505 | 28.834984 | 26.238739 | 25.818779 |
| FNA-T18 | 25.752193 | 29.163809 | 26.275812 | 25.886168 |
| FNA-T19 | 22.844242 | 27.09977 | 29.678366 | 24.440128 |
| FNA-T19 | 22.779215 | 27.06503 | 29.27977 | 24.542385 |
| FNA-T2 | 29.205395 | 27.61581 | 25.472637 | 23.16486 |
| FNA-T2 | 29.307468 | 27.85993 | 25.698673 | 23.274725 |
| FNA-T20 | 29.452393 | 25.752836 | 28.163557 | 19.300997 |
| FNA-T20 | 29.30013 | 25.739996 | 27.755651 | 19.213083 |
| FNA-T21 | 23.882595 | 26.676023 | 26.910845 | 23.411472 |
| FNA-T21 | 23.849615 | 26.81944 | 26.901575 | 23.554602 |
| FNA-T22 | 26.525532 | 27.649153 | 29.901728 | 22.168571 |
| FNA-T22 | 26.538591 | 27.584732 | 29.374508 | 22.292957 |
| FNA-T23 | 25.355679 | 25.70668 | 25.798903 | 20.72359 |
| FNA-T23 | 25.098497 | 25.781 | 25.591196 | 20.421762 |
| FNA-T24 | 31.655443 | 27.836647 | 29.566021 | 21.991768 |
| FNA-T24 | 31.494802 | 28.272316 | 29.88115 | 22.22226 |
| FNA-T25 | 29.921268 | 27.33637 | 27.2154 | 21.351002 |
| FNA-T25 | 30.059465 | 27.529253 | 27.259384 | 21.64143 |
| FNA-T26 | 30.944326 | 28.775505 | 28.623587 | 22.730553 |
| FNA-T26 | 30.618221 | 28.751554 | 28.746346 | 22.885683 |
| FNA-T27 | 29.5818 | 28.68144 | 28.439346 | 24.10121 |
| FNA-T27 | 29.748611 | 28.861916 | 28.642506 | 23.982159 |
| FNA-T28 | 30.305853 | 27.837112 | 25.967134 | 21.383492 |
| FNA-T28 | 30.086035 | 27.574003 | 25.797411 | 21.442741 |
| FNA-T29 | 30.520782 | 26.770647 | 29.989164 | 22.250774 |
| FNA-T29 | 30.314692 | 26.75742 | 30.370378 | 22.28899 |
| FNA-T3 | 30.725107 | 27.690231 | 29.522203 | 21.497208 |
| FNA-T3 | 30.949142 | 27.543365 | 29.654066 | 21.57565 |
| FNA-T30 | 30.807035 | 26.206888 | 29.31309 | 20.311813 |
| FNA-T30 | 30.943573 | 26.197397 | 29.412018 | 20.378456 |
| FNA-T31 | 29.3972 | 27.653997 | 27.73402 | 22.430922 |
| FNA-T31 | 28.985794 | 27.39575 | 27.860409 | 22.122993 |
| FNA-T32 | 32.19736 | 27.685831 | 31.223785 | 20.859383 |
| FNA-T32 | 32.456356 | 27.931112 | 31.86898 | 21.35533 |
| FNA-T33 | 29.434418 | 25.925068 | 29.998503 | 21.138245 |
| FNA-T33 | 30.254755 | 26.221485 | 30.233063 | 21.451023 |
| FNA-T34 | 30.67846 | 27.296001 | 29.32363 | 21.798595 |
| FNA-T34 | 31.00535 | 27.430798 | 29.584566 | 21.822613 |
| FNA-T35 | 30.513634 | 26.846313 | 28.36747 | 20.32238 |
| FNA-T35 | 30.618778 | 26.887613 | 28.317791 | 20.414942 |
| FNA-T36 | 32.082962 | 29.915424 | 29.71966 | 22.997276 |
| FNA-T36 | 32.090916 | 29.670103 | 29.26421 | 23.025518 |
| FNA-T37 | 30.94133 | 26.748241 | 28.763447 | 21.065388 |
| FNA-T37 | 31.25862 | 26.725689 | 29.277992 | 21.16831 |
| FNA-T38 | 34.353374 | 27.199493 | 28.505981 | 25.332064 |
| FNA-T38 | 34.774017 | 27.351727 | 28.497934 | 25.244667 |
| FNA-T39 | 29.368946 | 26.574303 | 26.915857 | 19.811405 |
| FNA-T39 | 29.085936 | 26.626427 | 27.14496 | 19.929224 |
| FNA-T4 | 29.275595 | 27.655647 | 25.40957 | 21.16699 |
| FNA-T4 | 29.526566 | 27.638578 | 25.32429 | 21.207483 |
| FNA-T40 | 31.564825 | 27.700035 | 29.315458 | 21.58901 |
| FNA-T40 | 31.643892 | 27.716877 | 28.884947 | 21.5213 |
| FNA-T41 | 30.922012 | 26.756815 | 28.485174 | 20.583536 |
| FNA-T41 | 30.98264 | 26.898712 | 28.77016 | 20.82358 |
| FNA-T42 | 22.755472 | 24.281397 | 24.092228 | 17.618967 |
| FNA-T42 | 22.523087 | 24.153524 | 23.84154 | 17.572567 |
| FNA-T43 | 21.671013 | 24.299393 | 26.105927 | 19.262407 |
| FNA-T43 | 21.783876 | 24.278673 | 25.992552 | 19.234812 |
| FNA-T44 | 21.151045 | 25.507265 | 27.515324 | 21.424637 |
| FNA-T44 | 21.030365 | 25.459078 | 27.542799 | 21.428375 |
| FNA-T45 | 27.29478 | 28.799063 | 31.007568 | 22.857424 |
| FNA-T45 | 27.359974 | 28.917706 | 31.876291 | 22.96709 |
| FNA-T46 | 24.300142 | 25.858658 | 28.52694 | 19.13223 |
| FNA-T46 | 21.728031 | 25.901863 | 28.569756 | 19.261362 |
| FNA-T47 | 25.53313 | 27.835642 | 30.756876 | 24.522694 |
| FNA-T47 | 25.800882 | 27.890217 | 30.398163 | 24.451723 |
| FNA-T48 | 23.571497 | 27.148775 | 29.730305 | 25.396801 |
| FNA-T48 | 23.46953 | 27.211584 | 30.114784 | 25.466698 |
| FNA-T49 | 22.075262 | 25.603071 | 25.10628 | 19.510986 |
| FNA-T49 | 21.893274 | 25.66718 | 25.01344 | 19.458557 |
| FNA-T5 | 28.515465 | 26.384024 | 26.45084 | 20.669962 |
| FNA-T5 | 28.667355 | 26.593912 | 26.410242 | 20.694485 |
| FNA-T50 | 23.493168 | 24.545803 | 27.133133 | 18.219131 |
| FNA-T50 | 23.74096 | 24.553919 | 27.315342 | 18.350636 |
| FNA-T51 | 23.605844 | 26.550951 | 29.172537 | 23.494295 |
| FNA-T51 | 23.763407 | 26.883968 | 29.42887 | 23.729527 |
| FNA-T52 | 23.694386 | 25.307146 | 25.719387 | 20.97136 |
| FNA-T52 | 23.636364 | 25.319677 | 25.82726 | 21.015167 |
| FNA-T53 | 30.59159 | 26.853363 | 28.739058 | 20.795107 |
| FNA-T53 | 31.237247 | 26.843718 | 28.837276 | 20.91187 |
| FNA-T54 | 31.353594 | 27.005735 | 30.271217 | 19.524746 |
| FNA-T54 | 31.541918 | 26.831339 | 30.824812 | 19.676071 |
| FNA-T55 | 29.907467 | 25.93737 | 28.899256 | 19.729116 |
| FNA-T55 | 30.54798 | 25.930876 | 28.859764 | 19.937914 |
| FNA-T56 | 32.533268 | 27.8079 | 31.30899 | 20.543272 |
| FNA-T56 | 32.76386 | 27.981945 | 31.675732 | 20.627884 |
| FNA-T57 | 26.819609 | 28.381905 | 30.426289 | 22.923357 |
| FNA-T57 | 26.662947 | 28.251654 | 30.812857 | 23.018944 |
| FNA-T58 | 29.776308 | 25.52434 | 27.486877 | 19.55204 |
| FNA-T58 | 29.873936 | 25.982357 | 27.818792 | 19.82805 |
| FNA-T59 | 24.736164 | 27.709654 | 27.119335 | 22.795462 |
| FNA-T59 | 24.635582 | 28.006199 | 27.107613 | 22.847567 |
| FNA-T6 | 33.183666 | 28.807083 | 31.282656 | 22.748539 |
| FNA-T6 | 33.672558 | 28.952227 | 31.653599 | 23.173307 |
| FNA-T60 | 31.64863 | 28.4077 | 31.640816 | 19.682795 |
| FNA-T60 | 31.589708 | 28.475708 | 31.197052 | 19.749275 |
| FNA-T61 | 32.605984 | 28.393358 | 29.68497 | 22.528143 |
| FNA-T61 | 32.77558 | 28.715664 | 30.435673 | 22.693125 |
| FNA-T62 | 31.788069 | 28.129845 | 30.736885 | 21.967327 |
| FNA-T62 | 32.36156 | 28.234833 | 30.76271 | 22.301533 |
| FNA-T7 | 26.470524 | 27.314032 | 25.543707 | 22.182627 |
| FNA-T7 | 26.692406 | 27.38797 | 25.587519 | 22.293215 |
| FNA-T8 | 32.04267 | 26.689198 | 25.81281 | 21.58136 |
| FNA-T8 | 24.715437 | 26.9592 | 25.496908 | 21.70281 |
| FNA-T9 | 34.81624 | 29.538303 | 32.40396 | 23.187382 |
| FNA-T9 | 34.547188 | 29.821249 | 32.94409 | 23.511566 |
| | miR- | | | |
| Sample ID | 181a-2-3p | miR-197-3p | miR-19b | miR-204-5p |
| FNA-T1 | 26.239046 | 23.791225 | 17.215101 | 29.735453 |
| FNA-T1 | 26.236746 | 23.952164 | 17.333632 | 30.533972 |
| FNA-T10 | 29.457657 | 26.932486 | 21.622456 | 33.77057 |
| FNA-T10 | 29.482769 | 26.577967 | 21.461847 | 33.342205 |
| FNA-T11 | 30.944098 | 26.777277 | 22.610968 | 35.810627 |
| FNA-T11 | 31.247454 | 26.703016 | 22.768736 | 34.085926 |
| FNA-T12 | 30.680006 | 29.121956 | 24.657997 | 33.690598 |
| FNA-T12 | 30.929245 | 28.512478 | 24.88813 | 34.141846 |
| FNA-T13 | 30.30806 | 27.272444 | 23.540218 | 32.731987 |
| FNA-T13 | 30.5223 | 27.231318 | 23.569202 | 32.784172 |
| FNA-T14 | 28.702656 | 27.975029 | 24.873064 | 32.2442 |
| FNA-T14 | 28.871883 | 28.399105 | 24.850115 | 31.91132 |
| FNA-T15 | 29.492765 | 24.925266 | 18.616865 | 29.414454 |
| FNA-T15 | 29.692642 | 24.977236 | 18.670918 | 29.272732 |
| FNA-T16 | 29.904036 | 28.346355 | 23.424759 | 32.863037 |
| FNA-T16 | 30.700163 | 28.162886 | 23.540562 | 31.896528 |
| FNA-T17 | 28.39143 | 26.315237 | 23.557722 | 31.1135 |
| FNA-T17 | 28.454716 | 26.630678 | 23.734999 | 31.160854 |
| FNA-T18 | 31.297668 | 27.64463 | 25.664581 | 32.427624 |
| FNA-T18 | 31.452131 | 27.652357 | 25.911089 | 32.542015 |
| FNA-T19 | 27.497894 | 26.844429 | 24.421219 | 30.931953 |
| FNA-T19 | 27.467567 | 27.216856 | 24.436321 | 30.70659 |
| FNA-T2 | 31.882801 | 27.329395 | 23.14852 | 30.513096 |
| FNA-T2 | 31.733582 | 27.27619 | 23.3389 | 30.86093 |
| FNA-T20 | 30.207386 | 26.991385 | 19.413057 | 26.810284 |
| FNA-T20 | 29.866316 | 26.816502 | 19.476957 | 26.615961 |
| FNA-T21 | 27.14218 | 26.105713 | 23.510206 | 30.704092 |
| FNA-T21 | 27.282236 | 26.233751 | 23.57707 | 30.993202 |
| FNA-T22 | 28.833063 | 28.603338 | 22.602016 | 33.345787 |
| FNA-T22 | 28.577223 | 28.574562 | 22.571129 | 33.382484 |
| FNA-T23 | 28.38265 | 26.353848 | 20.791359 | 31.733624 |
| FNA-T23 | 28.250664 | 26.188334 | 20.766026 | 31.856928 |
| FNA-T24 | 30.679192 | 28.10714 | 22.151901 | 27.877872 |
| FNA-T24 | 30.588951 | 28.108578 | 22.31967 | 27.731865 |
| FNA-T25 | 31.554508 | 26.076218 | 21.378325 | 28.55321 |
| FNA-T25 | 31.55156 | 26.29985 | 21.640467 | 28.69519 |
| FNA-T26 | 31.569275 | 27.612509 | 22.94717 | 29.080849 |
| FNA-T26 | 31.422535 | 27.653923 | 23.507088 | 29.325119 |
| FNA-T27 | 30.772295 | 27.185225 | 24.07127 | 29.74005 |
| FNA-T27 | 31.474485 | 26.879963 | 23.994165 | 29.915297 |
| FNA-T28 | 32.221565 | 27.801239 | 21.547987 | 30.10192 |
| FNA-T28 | 32.223766 | 27.523092 | 21.499022 | 30.081406 |
| FNA-T29 | 30.479488 | 26.960232 | 22.53613 | 24.089521 |
| FNA-T29 | 30.743345 | 26.991112 | 22.637787 | 23.956926 |
| FNA-T3 | 29.848053 | 27.721497 | 21.583067 | 28.12065 |
| FNA-T3 | 29.577131 | 27.96883 | 21.688324 | 28.204988 |
| FNA-T30 | 29.831251 | 27.168604 | 20.693306 | 26.063478 |
| FNA-T30 | 29.909483 | 27.596775 | 20.839579 | 26.348583 |
| FNA-T31 | 30.244614 | 26.145866 | 22.69481 | 27.859968 |
| FNA-T31 | 29.55648 | 25.980295 | 22.60781 | 27.642803 |
| FNA-T32 | 35.14281 | 28.980658 | 20.867188 | 29.37276 |
| FNA-T32 | 33.353256 | 29.262188 | 21.192448 | 29.305498 |
| FNA-T33 | 26.909027 | 26.601334 | 21.291773 | 24.374651 |
| FNA-T33 | 27.300104 | 26.71459 | 21.579449 | 24.600601 |
| FNA-T34 | 29.98888 | 27.061102 | 21.733154 | 25.86781 |
| FNA-T34 | 29.972055 | 27.103056 | 21.734434 | 25.870594 |
| FNA-T35 | 29.187536 | 27.233412 | 20.83039 | 26.77417 |
| FNA-T35 | 29.484724 | 27.14771 | 20.96967 | 26.794481 |
| FNA-T36 | 31.724634 | 28.590982 | 23.671719 | 29.853868 |
| FNA-T36 | 32.296886 | 28.40818 | 23.672071 | 29.847422 |
| FNA-T37 | 31.885494 | 25.628588 | 20.787703 | 30.26648 |
| FNA-T37 | 31.921024 | 25.494148 | 21.00772 | 30.246279 |
| FNA-T38 | 27.619493 | 27.23486 | 23.98677 | 28.797356 |
| FNA-T38 | 27.655455 | 26.96634 | 24.14323 | 29.008282 |
| FNA-T39 | 29.547138 | 26.197433 | 20.368402 | 30.471426 |
| FNA-T39 | 29.659397 | 26.216265 | 20.430325 | 30.163635 |
| FNA-T4 | 32.777424 | 27.100462 | 21.702898 | 31.907082 |
| FNA-T4 | 30.997013 | 26.942728 | 21.742785 | 31.801085 |
| FNA-T40 | 30.683807 | 27.295164 | 21.915226 | 27.718126 |
| FNA-T40 | 30.43888 | 27.420246 | 21.96684 | 27.923742 |
| FNA-T41 | 29.975554 | 26.63543 | 21.192091 | 29.858027 |
| FNA-T41 | 30.231993 | 26.663046 | 21.365736 | 29.771774 |
| FNA-T42 | 28.181196 | 25.088528 | 17.540688 | 28.736517 |
| FNA-T42 | 28.244263 | 24.969404 | 17.44289 | 28.689404 |
| FNA-T43 | 26.088053 | 25.679552 | 18.882652 | 29.305132 |
| FNA-T43 | 25.941563 | 25.684647 | 18.97493 | 29.32449 |
| FNA-T44 | 26.769348 | 26.412367 | 21.882946 | 27.958933 |
| FNA-T44 | 26.748264 | 26.336475 | 21.766151 | 27.72356 |
| FNA-T45 | 30.667646 | 26.161797 | 23.105068 | 32.117153 |
| FNA-T45 | 30.681046 | 26.34453 | 23.260078 | 33.20252 |
| FNA-T46 | 28.437214 | 25.97021 | 18.998446 | 30.353168 |
| FNA-T46 | 28.48834 | 26.142548 | 19.157124 | 30.663158 |
| FNA-T47 | 28.913853 | 28.50871 | 24.336916 | 29.648561 |
| FNA-T47 | 29.481346 | 28.616205 | 24.555311 | 29.680964 |
| FNA-T48 | 27.710281 | 26.409561 | 25.145098 | 30.204496 |
| FNA-T48 | 27.667358 | 26.227879 | 25.005325 | 30.325666 |
| FNA-T49 | 28.15067 | 26.486704 | 20.154232 | 29.780933 |
| FNA-T49 | 27.896036 | 26.33218 | 20.15728 | 29.964594 |
| FNA-T5 | 30.47705 | 25.745754 | 20.946533 | 28.334478 |
| FNA-T5 | 29.970358 | 25.89298 | 20.989868 | 28.421026 |
| FNA-T50 | 27.48278 | 25.590887 | 18.3425 | 30.62756 |
| FNA-T50 | 27.298765 | 25.611038 | 18.489569 | 30.58245 |
| FNA-T51 | 27.649504 | 25.991215 | 23.777613 | 29.084648 |
| FNA-T51 | 27.655264 | 26.229612 | 23.873423 | 29.259912 |
| FNA-T52 | 26.85997 | 26.158352 | 21.346916 | 27.838436 |
| FNA-T52 | 27.010382 | 26.081982 | 21.448828 | 27.755262 |
| FNA-T53 | 31.601341 | 26.518251 | 21.133186 | 28.466084 |
| FNA-T53 | 30.921926 | 26.512085 | 21.311647 | 28.488344 |
| FNA-T54 | 31.869246 | 27.203337 | 19.957684 | 30.296206 |
| FNA-T54 | 32.72795 | 26.949951 | 19.862558 | 30.151691 |
| FNA-T55 | 28.993805 | 26.964731 | 20.115822 | 25.171743 |
| FNA-T55 | 28.949444 | 26.89224 | 20.209393 | 25.200478 |
| FNA-T56 | 33.565117 | 29.159819 | 20.358751 | 29.953836 |
| FNA-T56 | 34.022488 | 29.426474 | 20.589308 | 30.399946 |
| FNA-T57 | 29.662863 | 28.54691 | 23.496655 | 28.020273 |
| FNA-T57 | 29.643246 | 28.857246 | 23.477638 | 27.831347 |
| FNA-T58 | 30.491571 | 27.49442 | 19.653465 | 27.744125 |
| FNA-T58 | 30.896376 | 27.697884 | 19.78614 | 27.77092 |
| FNA-T59 | 29.239832 | 27.87485 | 23.317558 | 29.15248 |
| FNA-T59 | 29.286037 | 27.791822 | 23.261757 | 28.877626 |
| FNA-T6 | 31.562769 | 27.687021 | 23.473692 | 27.709042 |
| FNA-T6 | 31.654436 | 27.844822 | 23.701212 | 28.101784 |
| FNA-T60 | 33.682297 | 29.32063 | 20.390467 | 30.341618 |
| FNA-T60 | 33.276573 | 29.490606 | 20.46086 | 30.88486 |
| FNA-T61 | 32.13518 | 28.759398 | 22.932732 | 30.297083 |
| FNA-T61 | 31.532003 | 28.680769 | 23.182756 | 30.131485 |
| FNA-T62 | 31.512766 | 27.54955 | 22.434282 | 27.451754 |
| FNA-T62 | 31.26316 | 27.66141 | 22.63109 | 27.578077 |
| FNA-T7 | 29.442358 | 26.65219 | 22.461542 | 28.639988 |
| FNA-T7 | 29.55025 | 26.73802 | 22.540699 | 28.737852 |
| FNA-T8 | 28.464312 | 26.31842 | 21.490355 | 28.839254 |
| FNA-T8 | 28.75942 | 26.371647 | 21.61542 | 28.951405 |
| FNA-T9 | 33.719215 | 27.948347 | 23.56581 | 32.99965 |
| FNA-T9 | 33.411324 | 28.453445 | 23.893435 | 33.132107 |
| Sample ID | miR-21-5p | miR-221-3p | miR-221-5p | miR-222-3p |
| FNA-T1 | 16.642141 | 21.357765 | 27.328987 | 19.84434 |
| FNA-T1 | 16.827057 | 21.320906 | 27.682508 | 19.934328 |
| FNA-T10 | 24.675112 | 24.95443 | 31.58463 | 23.74235 |
| FNA-T10 | 24.113333 | 24.584356 | 32.377167 | 23.405302 |
| FNA-T11 | 22.721924 | 26.949018 | 34.3314 | 25.289131 |
| FNA-T11 | 22.729729 | 27.275375 | 33.760258 | 25.51431 |
| FNA-T12 | 22.634512 | 27.818203 | 32.1954 | 24.979237 |
| FNA-T12 | 22.700262 | 27.654505 | 33.073463 | 25.243189 |
| FNA-T13 | 20.864109 | 25.921751 | 30.43412 | 23.137339 |
| FNA-T13 | 20.934944 | 25.892391 | 30.31687 | 23.168837 |
| FNA-T14 | 23.826445 | 25.625902 | 31.185017 | 22.814573 |
| FNA-T14 | 23.89024 | 25.501534 | 31.349373 | 22.960455 |
| FNA-T15 | 21.229485 | 24.675568 | 32.35197 | 22.79949 |
| FNA-T15 | 21.37636 | 25.012701 | 31.831976 | 22.992157 |
| FNA-T16 | 25.229063 | 26.102863 | 32.776455 | 24.140661 |
| FNA-T16 | 25.687212 | 26.261883 | 33.05607 | 24.013094 |
| FNA-T17 | 19.657827 | 23.859627 | 29.69458 | 21.10718 |
| FNA-T17 | 19.931728 | 24.147314 | 30.14789 | 21.13952 |
| FNA-T18 | 23.434616 | 26.542694 | 32.04296 | 23.872118 |
| FNA-T18 | 23.51714 | 26.584282 | 32.51727 | 23.973524 |
| FNA-T19 | 22.263987 | 23.083506 | 29.259949 | 21.22238 |
| FNA-T19 | 22.39362 | 23.09326 | 29.280416 | 21.326138 |
| FNA-T2 | 23.141502 | 28.573292 | 33.55477 | 24.909723 |
| FNA-T2 | 23.485144 | 28.964634 | 34.074646 | 24.951363 |
| FNA-T20 | 21.675373 | 26.97268 | 34.023212 | 24.428822 |
| FNA-T20 | 21.650827 | 27.002932 | 33.660378 | 24.438662 |
| FNA-T21 | 21.501682 | 22.936522 | 29.107313 | 21.613956 |
| FNA-T21 | 21.459675 | 23.002327 | 29.70916 | 21.77176 |
| FNA-T22 | 23.5476 | 26.353445 | 31.454437 | 23.23415 |
| FNA-T22 | 23.821466 | 26.58046 | 31.937336 | 23.269836 |
| FNA-T23 | 20.700804 | 24.308699 | 30.184607 | 21.62827 |
| FNA-T23 | 20.687664 | 23.913965 | 29.609146 | 21.513168 |
| FNA-T24 | 24.456676 | 27.275145 | 35.223473 | 25.721024 |
| FNA-T24 | 24.675978 | 27.216908 | 32.480167 | 25.836185 |
| FNA-T25 | 22.337307 | 27.707338 | 35.2058 | 25.78641 |
| FNA-T25 | 22.628887 | 27.837511 | 34.652565 | 26.108932 |
| FNA-T26 | 23.459728 | 29.18395 | 36.588284 | 27.531094 |
| FNA-T26 | 23.614176 | 28.833822 | 34.337036 | 27.636753 |
| FNA-T27 | 23.90884 | 27.75643 | 33.941227 | 26.774282 |
| FNA-T27 | 23.905787 | 27.815226 | 34.192028 | 26.74601 |
| FNA-T28 | 22.877193 | 27.924435 | 34.261456 | 25.577286 |
| FNA-T28 | 22.696705 | 27.899048 | 36.93079 | 25.635555 |
| FNA-T29 | 23.184736 | 29.689062 | 35.747486 | 27.892754 |
| FNA-T29 | 23.57018 | 28.800997 | 34.65945 | 28.123535 |
| FNA-T3 | 23.698166 | 25.691387 | 32.20123 | 24.62932 |
| FNA-T3 | 23.72975 | 25.809195 | 32.099625 | 24.723389 |
| FNA-T30 | 22.996277 | 27.110403 | 33.098713 | 25.531672 |
| FNA-T30 | 23.335133 | 27.591036 | 33.438843 | 25.614237 |
| FNA-T31 | 22.877148 | 27.972603 | 34.231426 | 26.108313 |
| FNA-T31 | 22.57646 | 27.563244 | 35.02869 | 25.890535 |
| FNA-T32 | 22.991724 | 29.918121 | 36.83741 | 26.331404 |
| FNA-T32 | 23.554337 | 29.858301 | 34.696888 | 26.720278 |
| FNA-T33 | 22.652767 | 26.75156 | 32.684772 | 24.467228 |
| FNA-T33 | 23.153368 | 26.948929 | 32.69105 | 24.734575 |
| FNA-T34 | 24.82618 | 27.553524 | 40.63565 | 26.731283 |
| FNA-T34 | 24.728811 | 27.462141 | 33.582413 | 26.801907 |
| FNA-T35 | 22.208548 | 28.331123 | 34.13096 | 25.430008 |
| FNA-T35 | 22.252235 | 27.985441 | 34.53764 | 25.61295 |
| FNA-T36 | 25.183472 | 30.225927 | 33.201927 | 27.863577 |
| FNA-T36 | 24.950567 | 30.059834 | 35.54011 | 28.013996 |
| FNA-T37 | 23.385887 | 23.876055 | 32.544136 | 24.389685 |
| FNA-T37 | 23.601929 | 23.781513 | 31.732666 | 24.442585 |
| FNA-T38 | 25.105978 | 28.277088 | 44.084072 | 28.350552 |
| FNA-T38 | 25.405413 | 28.242113 | 37.186676 | 28.320631 |
| FNA-T39 | 21.924557 | 24.837963 | 31.8528 | 24.39689 |
| FNA-T39 | 21.796032 | 24.59598 | 32.090496 | 24.547747 |
| FNA-T4 | 22.715698 | 28.274874 | 35.58193 | 25.211798 |
| FNA-T4 | 22.821077 | 27.842999 | 33.81601 | 25.196201 |
| FNA-T40 | 23.54718 | 28.342707 | 33.322765 | 26.382792 |
| FNA-T40 | 23.49541 | 28.207663 | 33.68603 | 26.462458 |
| FNA-T41 | 23.564583 | 26.790874 | 33.00083 | 24.239904 |
| FNA-T41 | 23.831728 | 27.141502 | 33.077003 | 24.403778 |
| FNA-T42 | 19.245577 | 23.818342 | 29.376299 | 21.657558 |
| FNA-T42 | 19.089708 | 23.519146 | 29.406467 | 21.68643 |
| FNA-T43 | 19.30529 | 22.529505 | 28.323366 | 20.567038 |
| FNA-T43 | 19.336924 | 22.543348 | 27.97006 | 20.546412 |
| FNA-T44 | 20.64032 | 24.332214 | 29.681082 | 22.725319 |
| FNA-T44 | 20.50007 | 24.247269 | 29.543419 | 22.663923 |
| FNA-T45 | 25.197552 | 27.791895 | 34.155445 | 24.951199 |
| FNA-T45 | 25.18148 | 27.723564 | 34.240948 | 24.932848 |
| FNA-T46 | 19.56883 | 23.798355 | 28.558037 | 21.285751 |
| FNA-T46 | 19.755262 | 23.709597 | 28.774336 | 21.349846 |
| FNA-T47 | 22.90814 | 27.10719 | 31.997103 | 23.9283 |
| FNA-T47 | 23.125473 | 27.094215 | 32.343838 | 24.242105 |
| FNA-T48 | 22.892971 | 25.35467 | 31.83498 | 25.638552 |
| FNA-T48 | 22.945427 | 25.36678 | 31.50254 | 23.506653 |
| FNA-T49 | 20.707172 | 23.742239 | 29.237608 | 22.254862 |
| FNA-T49 | 20.617254 | 23.52166 | 28.951166 | 22.199163 |
| FNA-T5 | 23.004347 | 26.354876 | 33.14333 | 24.221584 |
| FNA-T5 | 22.933012 | 26.438005 | 33.222958 | 24.293667 |
| FNA-T50 | 20.168625 | 23.361359 | 29.784513 | 21.717112 |
| FNA-T50 | 20.23227 | 23.493647 | 30.129198 | 21.810036 |
| FNA-T51 | 22.280159 | 23.474936 | 28.952478 | 21.141918 |
| FNA-T51 | 22.537466 | 23.656971 | 29.79213 | 21.390736 |
| FNA-T52 | 19.521214 | 23.643982 | 29.103622 | 20.875029 |
| FNA-T52 | 19.56719 | 23.674892 | 29.450514 | 21.157232 |
| FNA-T53 | 23.89266 | 27.53248 | 33.90312 | 25.43582 |
| FNA-T53 | 23.919025 | 27.62424 | 34.64586 | 25.45106 |
| FNA-T54 | 21.781326 | 28.855728 | 34.84435 | 25.821611 |
| FNA-T54 | 21.889166 | 28.484718 | 38.365856 | 25.661104 |
| FNA-T55 | 22.511087 | 26.242882 | 33.210567 | 24.59916 |
| FNA-T55 | 22.602274 | 26.341595 | 33.31462 | 24.636683 |
| FNA-T56 | 23.152048 | 29.841078 | 38.35461 | 26.945574 |
| FNA-T56 | 23.218822 | 29.634008 | 43.373234 | 26.9255 |
| FNA-T57 | 24.466152 | 25.980993 | 31.783796 | 24.486273 |
| FNA-T57 | 24.177647 | 25.949017 | 32.318287 | 24.434679 |
| FNA-T58 | 21.24823 | 24.67756 | 32.82988 | 24.2605 |
| FNA-T58 | 21.65543 | 24.903204 | 32.61433 | 24.43127 |
| FNA-T59 | 22.396976 | 25.416971 | 31.779005 | 23.40019 |
| FNA-T59 | 22.63583 | 25.438662 | 31.82377 | 23.452908 |
| FNA-T6 | 25.859325 | 29.61415 | 34.542934 | 27.96887 |
| FNA-T6 | 26.25842 | 29.986795 | 35.714413 | 28.3271 |
| FNA-T60 | 22.91946 | 29.56167 | 34.83162 | 26.991592 |
| FNA-T60 | 22.710762 | 29.77171 | 33.800293 | 26.837193 |
| FNA-T61 | 25.105469 | 27.433054 | 34.742928 | 25.974476 |
| FNA-T61 | 25.28259 | 27.345282 | 34.662525 | 25.993118 |
| FNA-T62 | 24.525728 | 29.262197 | 34.50554 | 27.409775 |
| FNA-T62 | 24.807543 | 29.599257 | Undetermined | 27.668844 |
| FNA-T7 | 23.22485 | 25.900362 | 31.472517 | 22.983604 |
| FNA-T7 | 23.366869 | 26.269121 | 32.093784 | 23.153976 |
| FNA-T8 | 22.465874 | 24.141613 | 30.325752 | 22.532228 |
| FNA-T8 | 22.562605 | 24.29681 | 31.401922 | 22.579184 |
| FNA-T9 | 26.539865 | 28.935747 | 34.833427 | 27.334845 |
| FNA-T9 | 27.228676 | 29.6814 | 34.584236 | 27.69754 |
| Sample ID | miR-29b-1* | miR-29b-3p | miR-31-5p | miR-328 |
| FNA-T1 | 26.552824 | 22.407076 | 21.674368 | 24.717823 |
| FNA-T1 | 26.738533 | 22.68818 | 21.679962 | 24.76487 |
| FNA-T10 | 29.578978 | 27.923645 | 27.209602 | 26.714931 |
| FNA-T10 | 30.104855 | 27.636387 | 26.607618 | 26.492651 |
| FNA-T11 | 29.801655 | 28.82855 | 29.220049 | 28.52914 |
| FNA-T11 | 30.33799 | 28.94199 | 28.905233 | 28.277044 |
| FNA-T12 | 31.445667 | 27.562014 | 26.282423 | 28.423443 |
| FNA-T12 | 32.609802 | 27.495533 | 26.384924 | 28.415081 |
| FNA-T13 | 30.620462 | 26.155315 | 25.77704 | 27.752846 |
| FNA-T13 | 31.158028 | 26.07141 | 25.856716 | 27.903645 |
| FNA-T14 | 30.408047 | 27.230663 | 25.737677 | 27.310417 |
| FNA-T14 | 30.44525 | 27.425661 | 25.86754 | 27.442314 |
| FNA-T15 | 26.69902 | 25.444492 | 29.782415 | 25.196224 |
| FNA-T15 | 26.83123 | 25.58293 | 29.693596 | 25.3076 |
| FNA-T16 | 30.895475 | 28.958227 | 27.21757 | 27.747417 |
| FNA-T16 | 31.24299 | 29.210745 | 27.194315 | 27.5524 |
| FNA-T17 | 28.573452 | 25.898495 | 24.39711 | 27.148203 |
| FNA-T17 | 28.704588 | 26.101818 | 24.908726 | 27.162281 |
| FNA-T18 | 30.857159 | 28.79546 | 26.306192 | 28.122135 |
| FNA-T18 | 30.89154 | 28.616695 | 26.114096 | 28.193056 |
| FNA-T19 | 28.924171 | 26.516384 | 24.16467 | 25.929075 |
| FNA-T19 | 28.8763 | 26.657063 | 24.253307 | 25.996042 |
| FNA-T2 | 30.307966 | 27.803764 | 27.817917 | 28.397722 |
| FNA-T2 | 30.565325 | 27.856993 | 27.89706 | 28.562277 |
| FNA-T20 | 27.164104 | 25.318382 | 31.214188 | 26.014467 |
| FNA-T20 | 27.47511 | 25.2455 | 31.34887 | 25.94132 |
| FNA-T21 | 29.29395 | 26.474457 | 25.319622 | 26.095268 |
| FNA-T21 | 28.993692 | 26.63563 | 25.361935 | 26.132647 |
| FNA-T22 | 29.889164 | 28.32289 | 26.998734 | 27.714632 |
| FNA-T22 | 29.91072 | 28.499876 | 26.964794 | 27.659988 |
| FNA-T23 | 28.428488 | 26.144009 | 24.765995 | 26.547787 |
| FNA-T23 | 28.223621 | 25.56995 | 24.842922 | 26.389767 |
| FNA-T24 | 29.733946 | 28.780647 | 29.8842 | 27.809406 |
| FNA-T24 | 29.783478 | 28.941866 | 30.482067 | 27.920282 |
| FNA-T25 | 29.509336 | 28.153896 | 31.00858 | 27.541487 |
| FNA-T25 | 29.577816 | 28.66477 | 31.974932 | 27.854446 |
| FNA-T26 | 30.795837 | 29.46229 | 30.09425 | 27.782421 |
| FNA-T26 | 30.34819 | 29.380663 | 30.183193 | 28.158354 |
| FNA-T27 | 30.583546 | 29.679585 | 30.389462 | 27.31657 |
| FNA-T27 | 30.138546 | 29.180809 | 29.975212 | 27.275612 |
| FNA-T28 | 29.447802 | 26.958107 | 29.224815 | 27.570503 |
| FNA-T28 | 29.3097 | 26.708725 | 29.14273 | 27.437054 |
| FNA-T29 | 28.604471 | 26.42835 | 32.183193 | 27.199223 |
| FNA-T29 | 28.736488 | 26.497454 | 32.12903 | 27.127747 |
| FNA-T3 | 29.660929 | 27.963284 | 30.645727 | 27.5603 |
| FNA-T3 | 29.733301 | 28.240019 | 31.523262 | 27.765686 |
| FNA-T30 | 28.946253 | 27.141096 | 29.745821 | 26.90276 |
| FNA-T30 | 28.720947 | 27.340471 | 29.74179 | 26.82381 |
| FNA-T31 | 29.354944 | 28.903929 | 31.692434 | 26.424232 |
| FNA-T31 | 29.10675 | 28.486921 | 31.458744 | 26.556625 |
| FNA-T32 | 30.756907 | 27.51954 | 33.502304 | 29.329964 |
| FNA-T32 | 31.592253 | 27.713757 | 32.873417 | 29.71085 |
| FNA-T33 | 28.117823 | 26.871046 | 31.84709 | 25.612028 |
| FNA-T33 | 28.306261 | 27.167936 | 32.161015 | 25.84176 |
| FNA-T34 | 28.335701 | 27.505013 | 30.548182 | 26.832096 |
| FNA-T34 | 28.002518 | 27.479843 | 30.362991 | 26.855673 |
| FNA-T35 | 28.900257 | 27.314783 | 29.010115 | 26.724215 |
| FNA-T35 | 29.005304 | 27.63258 | 29.713867 | 26.932941 |
| FNA-T36 | 30.766787 | 29.719513 | 31.582434 | 29.072824 |
| FNA-T36 | 30.898794 | 30.086916 | 32.46275 | 29.225729 |
| FNA-T37 | 28.643822 | 27.933632 | 30.65435 | 25.64595 |
| FNA-T37 | 29.121542 | 27.585506 | 30.842812 | 25.72746 |
| FNA-T38 | 32.501987 | 30.83824 | 31.608345 | 29.263035 |
| FNA-T38 | 32.377304 | 30.838232 | 31.4355 | 28.796772 |
| FNA-T39 | 28.469568 | 26.985947 | 28.790743 | 26.430431 |
| FNA-T39 | 28.394993 | 27.003155 | 28.892612 | 26.325079 |
| FNA-T4 | 29.70096 | 27.641838 | 27.997614 | 27.788136 |
| FNA-T4 | 30.089777 | 27.48301 | 28.204514 | 27.51864 |
| FNA-T40 | 29.535404 | 28.631895 | 32.209587 | 27.045666 |
| FNA-T40 | 29.340818 | 28.945646 | 31.332457 | 26.892359 |
| FNA-T41 | 28.513926 | 27.531015 | 31.700697 | 26.606771 |
| FNA-T41 | 28.697788 | 27.763035 | 31.34682 | 26.520512 |
| FNA-T42 | 27.17159 | 23.768936 | 24.67187 | 25.634274 |
| FNA-T42 | 27.221664 | 23.780148 | 24.692028 | 25.479988 |
| FNA-T43 | 27.311766 | 24.51567 | 23.661364 | 25.720545 |
| FNA-T43 | 26.971657 | 24.44387 | 23.408943 | 25.807112 |
| FNA-T44 | 28.614473 | 25.378832 | 24.522394 | 25.804615 |
| FNA-T44 | 28.594675 | 25.456528 | 24.322575 | 25.710333 |
| FNA-T45 | 31.530228 | 29.795317 | 27.698383 | 26.393347 |
| FNA-T45 | 31.391783 | 29.712856 | 27.829576 | 26.452871 |
| FNA-T46 | 28.797535 | 23.662062 | 23.625675 | 25.876642 |
| FNA-T46 | 28.800667 | 23.96831 | 23.391968 | 25.873774 |
| FNA-T47 | 30.460367 | 28.372406 | 26.189934 | 27.689249 |
| FNA-T47 | 30.539267 | 28.137783 | 26.433525 | 27.787106 |
| FNA-T48 | 29.636694 | 28.251192 | 25.172813 | 25.575579 |
| FNA-T48 | 29.467516 | 28.492643 | 25.152712 | 25.585772 |
| FNA-T49 | 28.196089 | 24.9778 | 23.246456 | 25.784502 |
| FNA-T49 | 27.903898 | 24.805834 | 23.341095 | 25.622856 |
| FNA-T5 | 28.81858 | 27.08423 | 28.870111 | 26.408035 |
| FNA-T5 | 28.892872 | 27.171904 | 28.904049 | 26.230465 |
| FNA-T50 | 27.892622 | 25.304169 | 24.839092 | 25.700035 |
| FNA-T50 | 27.809248 | 25.495163 | 24.692587 | 25.635054 |
| FNA-T51 | 28.804409 | 26.369228 | 24.379492 | 25.550999 |
| FNA-T51 | 29.089012 | 26.880339 | 24.514566 | 25.779247 |
| FNA-T52 | 27.879488 | 25.62345 | 23.779797 | 26.477188 |
| FNA-T52 | 28.457241 | 25.661802 | 23.788904 | 26.412746 |
| FNA-T53 | 29.229086 | 28.122553 | 30.460966 | 26.603947 |
| FNA-T53 | 29.080864 | 28.127106 | 30.35179 | 26.729425 |
| FNA-T54 | 30.072956 | 26.63474 | 31.593695 | 27.151697 |
| FNA-T54 | 30.172771 | 26.431742 | 31.581427 | 27.194582 |
| FNA-T55 | 28.083344 | 26.968813 | 30.337082 | 26.680065 |
| FNA-T55 | 28.19728 | 26.960432 | 29.65397 | 26.706018 |
| FNA-T56 | 30.934877 | 27.728243 | 33.710785 | 28.976395 |
| FNA-T56 | 31.28978 | 28.126823 | 33.374626 | 29.97552 |
| FNA-T57 | 30.093792 | 27.307894 | 28.888605 | 27.902843 |
| FNA-T57 | 29.88062 | 27.230942 | 28.731913 | 27.984482 |
| FNA-T58 | 27.730694 | 25.952473 | 31.333189 | 27.171295 |
| FNA-T58 | 27.828024 | 26.051939 | 30.879128 | 27.379938 |
| FNA-T59 | 29.22478 | 27.990944 | 25.566982 | 26.99639 |
| FNA-T59 | 29.110918 | 27.79271 | 25.547623 | 27.220053 |
| FNA-T6 | 30.151512 | 29.729553 | 32.15644 | 26.919432 |
| FNA-T6 | 30.607885 | 29.989788 | 33.895775 | 27.135128 |
| FNA-T60 | 30.099794 | 25.85505 | 32.308018 | 29.690987 |
| FNA-T60 | 30.52286 | 25.911158 | 31.561152 | 29.411201 |
| FNA-T61 | 31.107653 | 28.86514 | 29.17722 | 28.659204 |
| FNA-T61 | 30.813538 | 29.242283 | 29.512753 | 28.473232 |
| FNA-T62 | 29.332699 | 28.253344 | 31.560658 | 26.864761 |
| FNA-T62 | 29.59081 | 28.642502 | 32.277462 | 27.004774 |
| FNA-T7 | 29.505049 | 27.154976 | 25.201332 | 26.64349 |
| FNA-T7 | 29.54286 | 27.272453 | 25.293428 | 26.27916 |
| FNA-T8 | 28.886976 | 26.35394 | 24.529758 | 25.89689 |
| FNA-T8 | 28.988264 | 26.36036 | 24.831955 | 26.221567 |
| FNA-T9 | 31.547228 | 30.27583 | 32.58279 | 28.348913 |
| FNA-T9 | 31.12415 | 30.882647 | 33.22385 | 28.463833 |
| Sample ID | miR-34a | miR-375 | miR-424-5p | miR-551b-3p |
| FNA-T1 | 21.759861 | 24.357325 | 21.689108 | 23.92916 |
| FNA-T1 | 21.90121 | 24.52902 | 21.716686 | 24.213411 |
| FNA-T10 | 26.714418 | 26.906414 | 27.610638 | 30.333134 |
| FNA-T10 | 26.395859 | 26.84903 | 27.288382 | 29.312304 |
| FNA-T11 | 27.571136 | 33.668278 | 27.786442 | 32.735714 |
| FNA-T11 | 27.814194 | 33.161793 | 28.25139 | 33.91899 |
| FNA-T12 | 26.743275 | 29.538189 | 25.58171 | 28.615417 |
| FNA-T12 | 26.97743 | 29.535969 | 25.793852 | 28.848145 |
| FNA-T13 | 26.32446 | 28.943882 | 25.823872 | 27.212582 |
| FNA-T13 | 25.184324 | 28.790844 | 26.065561 | 27.57199 |
| FNA-T14 | 26.87068 | 28.105324 | 26.995684 | 27.912191 |
| FNA-T14 | 26.807198 | 28.220304 | 27.383945 | 27.82647 |
| FNA-T15 | 25.972559 | 30.297363 | 23.469584 | 30.29642 |
| FNA-T15 | 26.147264 | 30.960415 | 23.55051 | 31.237104 |
| FNA-T16 | 28.341677 | 28.389948 | 26.23162 | 29.417755 |
| FNA-T16 | 28.408524 | 28.559496 | 26.427473 | 29.275133 |
| FNA-T17 | 23.863186 | 26.5023 | 26.409435 | 27.33214 |
| FNA-T17 | 24.100197 | 26.646877 | 26.79651 | 27.65021 |
| FNA-T18 | 27.613903 | 28.427395 | 28.244299 | 29.320124 |
| FNA-T18 | 27.99891 | 28.254398 | 28.21038 | 29.568445 |
| FNA-T19 | 25.155092 | 24.98625 | 24.376642 | 26.307323 |
| FNA-T19 | 25.408289 | 24.948767 | 24.655384 | 26.65667 |
| FNA-T2 | 28.846933 | 32.218384 | 27.797487 | 32.396782 |
| FNA-T2 | 28.657774 | 32.25273 | 28.133684 | 33.658894 |
| FNA-T20 | 26.521584 | 27.958426 | 24.983604 | 34.49795 |
| FNA-T20 | 26.793703 | 27.941505 | 25.087746 | 31.87205 |
| FNA-T21 | 24.22911 | 27.440147 | 25.571928 | 26.844156 |
| FNA-T21 | 24.335741 | 27.470142 | 25.725945 | 27.166061 |
| FNA-T22 | 26.558506 | 29.921537 | 27.155134 | 29.121315 |
| FNA-T22 | 26.83183 | 29.812838 | 27.424238 | 29.771072 |
| FNA-T23 | 24.26695 | 29.322336 | 26.358822 | 27.758314 |
| FNA-T23 | 23.75239 | 29.62862 | 26.281637 | 27.847853 |
| FNA-T24 | 28.781298 | 33.359737 | 27.35114 | 31.791058 |
| FNA-T24 | 28.783527 | 32.31928 | 27.85144 | 32.25515 |
| FNA-T25 | 27.217218 | 34.218094 | 27.433104 | 39.353657 |
| FNA-T25 | 27.640055 | 35.411415 | 28.298134 | 33.99329 |
| FNA-T26 | 28.286097 | 35.812702 | 27.71359 | 34.886494 |
| FNA-T26 | 28.192442 | 34.335247 | 28.346489 | 33.58086 |
| FNA-T27 | 27.707132 | 34.855465 | 29.58477 | 35.19459 |
| FNA-T27 | 27.739868 | 35.77197 | 29.687668 | 35.304783 |
| FNA-T28 | 28.52387 | 34.582108 | 26.444414 | 34.218353 |
| FNA-T28 | 28.180721 | 33.574974 | 26.334066 | 33.263306 |
| FNA-T29 | 25.505648 | 34.221226 | 25.376543 | 35.334885 |
| FNA-T29 | 25.576519 | 33.386536 | 25.414387 | 34.437332 |
| FNA-T3 | 26.475283 | 27.948328 | 25.575031 | 33.1651 |
| FNA-T3 | 26.72403 | 27.878012 | 25.800873 | 33.890945 |
| FNA-T30 | 25.80545 | 34.008995 | 25.832836 | 34.11272 |
| FNA-T30 | 26.115345 | 34.093388 | 26.072142 | 33.140255 |
| FNA-T31 | 27.499578 | 32.945213 | 27.39106 | 32.463623 |
| FNA-T31 | 27.16642 | 34.386936 | 26.962072 | 33.497566 |
| FNA-T32 | 31.421667 | 33.854218 | 25.922308 | 32.580975 |
| FNA-T32 | 32.63763 | 35.10524 | 26.213865 | 34.909473 |
| FNA-T33 | 26.306517 | 37.614838 | 22.88349 | 33.628967 |
| FNA-T33 | 26.454823 | 35.20879 | 23.211344 | 34.75065 |
| FNA-T34 | 26.449413 | 33.447548 | 26.982445 | 32.989803 |
| FNA-T34 | 26.341072 | 33.379044 | 26.879694 | 35.20106 |
| FNA-T35 | 26.968714 | 29.135952 | 25.497559 | 33.308086 |
| FNA-T35 | 27.189608 | 29.120487 | 25.715252 | 32.951935 |
| FNA-T36 | 28.91105 | 34.953197 | 27.69415 | 34.817013 |
| FNA-T36 | 28.894653 | 33.975353 | 27.690119 | 33.352234 |
| FNA-T37 | 26.910515 | 35.39039 | 26.13929 | 33.478268 |
| FNA-T37 | 26.862045 | 41.395145 | 26.40512 | 32.866364 |
| FNA-T38 | 28.332905 | 36.336166 | 27.403008 | 33.263515 |
| FNA-T38 | 28.450504 | 36.306988 | 27.721601 | 37.213985 |
| FNA-T39 | 26.509357 | 31.333836 | 25.737087 | 32.973557 |
| FNA-T39 | 26.499653 | 31.396465 | 25.934643 | 33.704407 |
| FNA-T4 | 28.71147 | 31.783518 | 27.354052 | 31.642044 |
| FNA-T4 | 28.673788 | 31.917233 | 27.443644 | 32.118744 |
| FNA-T40 | 28.10988 | 35.244644 | 26.687002 | 31.886219 |
| FNA-T40 | 27.704485 | 34.488426 | 26.902958 | 33.005867 |
| FNA-T41 | 28.116488 | 30.461643 | 26.472574 | 33.29255 |
| FNA-T41 | 28.713839 | 31.137833 | 26.642555 | 32.95098 |
| FNA-T42 | 24.331818 | 26.979107 | 23.70171 | 26.858057 |
| FNA-T42 | 24.23564 | 26.843723 | 23.762442 | 26.73503 |
| FNA-T43 | 23.246593 | 25.80915 | 23.447712 | 25.407848 |
| FNA-T43 | 23.482557 | 25.90109 | 23.405668 | 25.314327 |
| FNA-T44 | 24.191206 | 24.123878 | 22.833368 | 26.322565 |
| FNA-T44 | 24.20323 | 24.072432 | 22.768677 | 26.346481 |
| FNA-T45 | 28.792751 | 29.191832 | 27.903502 | 30.956787 |
| FNA-T45 | 28.881836 | 29.281704 | 28.120775 | 30.604996 |
| FNA-T46 | 24.505272 | 26.103832 | 24.481792 | 25.292429 |
| FNA-T46 | 24.524801 | 26.198767 | 24.738825 | 25.27952 |
| FNA-T47 | 25.661707 | 27.970852 | 25.671757 | 28.870934 |
| FNA-T47 | 25.525518 | 28.165735 | 25.975157 | 29.276144 |
| FNA-T48 | 25.584394 | 25.316916 | 26.86571 | 28.38578 |
| FNA-T48 | 25.43255 | 25.348103 | 27.139637 | 28.41595 |
| FNA-T49 | 24.199627 | 25.421167 | 24.435448 | 26.12788 |
| FNA-T49 | 24.128614 | 25.343431 | 24.296907 | 26.10545 |
| FNA-T5 | 28.22904 | 28.223204 | 25.668484 | 32.015877 |
| FNA-T5 | 28.130407 | 28.367552 | 25.708902 | 31.857765 |
| FNA-T50 | 24.635742 | 26.419092 | 23.418978 | 27.62377 |
| FNA-T50 | 24.799103 | 26.599754 | 23.560799 | 27.477293 |
| FNA-T51 | 24.850182 | 25.827854 | 25.093607 | 27.454805 |
| FNA-T51 | 25.178524 | 26.155672 | 25.549541 | 27.436525 |
| FNA-T52 | 23.40972 | 27.213345 | 23.948849 | 26.530197 |
| FNA-T52 | 23.415026 | 27.178568 | 24.14129 | 26.547876 |
| FNA-T53 | 29.006569 | 31.934603 | 26.50384 | 33.893867 |
| FNA-T53 | 30.00482 | 32.228584 | 26.785177 | 33.594067 |
| FNA-T54 | 30.230719 | 35.55649 | 24.54654 | 33.81531 |
| FNA-T54 | 30.348034 | 33.777855 | 24.50558 | 33.37074 |
| FNA-T55 | 27.150486 | 30.397959 | 26.60232 | 32.5844 |
| FNA-T55 | 27.450245 | 30.535732 | 26.84302 | 33.873325 |
| FNA-T56 | 30.826586 | 33.905197 | 25.787544 | 35.205944 |
| FNA-T56 | 31.207478 | 34.584137 | 26.272888 | 35.307472 |
| FNA-T57 | 27.335266 | 25.708355 | 24.945887 | 29.157518 |
| FNA-T57 | 27.388218 | 26.221941 | 24.756659 | 28.935549 |
| FNA-T58 | 29.32769 | 31.663275 | 24.78945 | 32.03856 |
| FNA-T58 | 29.572084 | 31.539835 | 25.112747 | 31.817013 |
| FNA-T59 | 26.361597 | 26.910456 | 25.969242 | 28.525698 |
| FNA-T59 | 26.52823 | 26.91924 | 26.02117 | 28.516512 |
| FNA-T6 | 29.114359 | 35.416687 | 27.748613 | Undetermined |
| FNA-T6 | 29.507107 | 37.737366 | 27.9832 | 38.977604 |
| FNA-T60 | 29.171234 | 32.740345 | 24.909168 | 34.16094 |
| FNA-T60 | 29.369196 | 33.4151 | 24.969099 | 33.008026 |
| FNA-T61 | 27.463772 | 30.406023 | 27.430864 | 36.95841 |
| FNA-T61 | 27.75444 | 30.957478 | 27.97967 | 33.324726 |
| FNA-T62 | 27.915558 | 27.626045 | 26.68034 | 28.60977 |
| FNA-T62 | 28.15422 | 27.920708 | 27.069723 | 28.961477 |
| FNA-T7 | 27.875864 | 28.565523 | 26.22175 | 29.542273 |
| FNA-T7 | 27.975122 | 28.598747 | 26.411266 | 29.828125 |
| FNA-T8 | 25.715467 | 28.33554 | 24.43361 | 30.01527 |
| FNA-T8 | 25.809011 | 28.592302 | 25.037632 | 30.422173 |
| FNA-T9 | 30.436687 | 32.909435 | 28.329449 | Undetermined |
| FNA-T9 | 31.15085 | 35.34046 | 28.869225 | 33.567963 |

| Sample ID | miR-7-5p | miR-96-5p |
|---|---|---|
| FNA-T1 | 24.46035 | 26.107105 |
| FNA-T1 | 24.46492 | 26.13061 |
| FNA-T10 | 27.78626 | 30.723984 |
| FNA-T10 | 28.47452 | 29.596931 |
| FNA-T11 | 30.17391 | 30.918604 |
| FNA-T11 | 29.90885 | 30.655432 |
| FNA-T12 | 33.76864 | 32.239414 |
| FNA-T12 | 32.76183 | 32.28395 |
| FNA-T13 | 31.15464 | 31.583239 |
| FNA-T13 | 30.68807 | 31.939806 |
| FNA-T14 | 32.40135 | 32.337006 |
| FNA-T14 | 32.8548 | 32.111927 |
| FNA-T15 | 25.17525 | 28.431435 |
| FNA-T15 | 25.40695 | 27.549498 |
| FNA-T16 | 31.49821 | 31.727325 |
| FNA-T16 | 31.56099 | 31.483181 |
| FNA-T17 | 30.2633 | 29.865303 |
| FNA-T17 | 31.10866 | 29.800121 |
| FNA-T18 | 32.07932 | 34.49733 |
| FNA-T18 | 31.75947 | 33.630657 |
| FNA-T19 | 31.04584 | 32.60601 |
| FNA-T19 | 31.57937 | 33.249607 |
| FNA-T2 | 30.74412 | 30.705088 |
| FNA-T2 | 30.01509 | 31.319689 |
| FNA-T20 | 23.31374 | 27.27577 |
| FNA-T20 | 23.23589 | 27.185717 |
| FNA-T21 | 31.33283 | 29.375444 |
| FNA-T21 | 31.54454 | 29.20065 |
| FNA-T22 | 30.41792 | 29.98111 |
| FNA-T22 | 30.24154 | 30.21693 |
| FNA-T23 | 28.48276 | 28.855429 |
| FNA-T23 | 28.40838 | 28.46479 |
| FNA-T24 | 28.24032 | 30.539133 |
| FNA-T24 | 28.43015 | 30.84059 |
| FNA-T25 | 25.88101 | 29.10208 |
| FNA-T25 | 26.35999 | 29.604494 |
| FNA-T26 | 30.37015 | 31.170195 |
| FNA-T26 | 30.67165 | 30.97432 |
| FNA-T27 | 28.9647 | 32.97908 |
| FNA-T27 | 28.80167 | 32.51327 |
| FNA-T28 | 28.5642 | 29.83185 |
| FNA-T28 | 28.76439 | 29.795675 |
| FNA-T29 | 24.70477 | 28.38451 |
| FNA-T29 | 24.92562 | 28.464401 |
| FNA-T3 | 28.43561 | 29.757824 |
| FNA-T3 | 27.89716 | 30.203068 |
| FNA-T30 | 27.13216 | 27.968243 |
| FNA-T30 | 27.25848 | 28.32718 |
| FNA-T31 | 25.50765 | 32.13602 |
| FNA-T31 | 25.57574 | 31.709713 |
| FNA-T32 | 26.87805 | 29.005896 |
| FNA-T32 | 27.01653 | 29.350685 |
| FNA-T33 | 23.21308 | 29.309095 |
| FNA-T33 | 23.43962 | 29.411545 |
| FNA-T34 | 28.19318 | 29.412329 |
| FNA-T34 | 28.30631 | 29.23639 |
| FNA-T35 | 24.64907 | 28.87817 |
| FNA-T35 | 25.09017 | 29.19014 |
| FNA-T36 | 26.9485 | 31.188734 |
| FNA-T36 | 27.21083 | 30.639658 |
| FNA-T37 | 28.58959 | 29.905659 |
| FNA-T37 | 28.56261 | 29.908806 |
| FNA-T38 | 32.7213 | 34.21291 |
| FNA-T38 | 32.79255 | 32.709476 |
| FNA-T39 | 27.66424 | 27.81202 |
| FNA-T39 | 27.0982 | 28.013802 |
| FNA-T4 | 29.55808 | 29.872852 |
| FNA-T4 | 29.50446 | 29.952682 |
| FNA-T40 | 25.90843 | 29.320099 |
| FNA-T40 | 25.76527 | 29.29864 |
| FNA-T41 | 28.73733 | 27.673727 |
| FNA-T41 | 28.13154 | 28.199219 |
| FNA-T42 | 25.21124 | 26.567322 |
| FNA-T42 | 24.37088 | 26.479298 |
| FNA-T43 | 26.84822 | 26.861986 |
| FNA-T43 | 26.34531 | 26.84644 |
| FNA-T44 | 27.92295 | 29.3795 |
| FNA-T44 | 27.80024 | 29.724712 |
| FNA-T45 | 30.70461 | 32.183765 |
| FNA-T45 | 31.41038 | 31.81356 |
| FNA-T46 | 26.84471 | 27.751984 |
| FNA-T46 | 27.36969 | 27.85507 |
| FNA-T47 | 30.30388 | 29.571192 |
| FNA-T47 | 30.89902 | 30.149002 |
| FNA-T48 | 33.7549 | 32.988167 |
| FNA-T48 | 33.52822 | 33.74298 |
| FNA-T49 | 26.93898 | 28.287607 |
| FNA-T49 | 26.73706 | 28.23724 |
| FNA-T5 | 27.4728 | 28.540136 |
| FNA-T5 | 27.54598 | 28.507719 |
| FNA-T50 | 24.57541 | 26.98736 |
| FNA-T50 | 24.5551 | 27.314873 |
| FNA-T51 | 29.96059 | 31.82041 |
| FNA-T51 | 30.48503 | 32.517166 |
| FNA-T52 | 28.43395 | 27.556486 |
| FNA-T52 | 28.2359 | 27.767237 |
| FNA-T53 | 25.96144 | 29.29252 |
| FNA-T53 | 25.79281 | 29.348373 |
| FNA-T54 | 27.14429 | 28.32788 |
| FNA-T54 | 27.42552 | 27.934515 |
| FNA-T55 | 26.18757 | 28.257067 |
| FNA-T55 | 26.38634 | 28.369804 |
| FNA-T56 | 27.93556 | 30.851402 |
| FNA-T56 | 28.46733 | 30.99064 |
| FNA-T57 | 28.94717 | 30.359634 |
| FNA-T57 | 28.76204 | 29.979872 |
| FNA-T58 | 26.3276 | 28.790974 |
| FNA-T58 | 26.76476 | 29.1504 |
| FNA-T59 | 29.35478 | 30.772585 |
| FNA-T59 | 29.91906 | 30.500744 |
| FNA-T6 | 27.30776 | 31.164837 |
| FNA-T6 | 27.9615 | 31.79102 |
| FNA-T60 | 27.92587 | 27.634392 |
| FNA-T60 | 27.93238 | 27.844763 |
| FNA-T61 | 25.7991 | 30.445461 |
| FNA-T61 | 26.34176 | 30.246597 |
| FNA-T62 | 26.68377 | 29.931646 |
| FNA-T62 | 26.64297 | 30.5349 |
| FNA-T7 | 29.85429 | 30.101599 |
| FNA-T7 | 29.80737 | 30.094799 |
| FNA-T8 | 29.26844 | 28.872662 |
| FNA-T8 | 29.68719 | 29.60882 |
| FNA-T9 | 30.77081 | 32.787815 |
| FNA-T9 | 31.20988 | 32.57085 |

### FNA Classifier Results

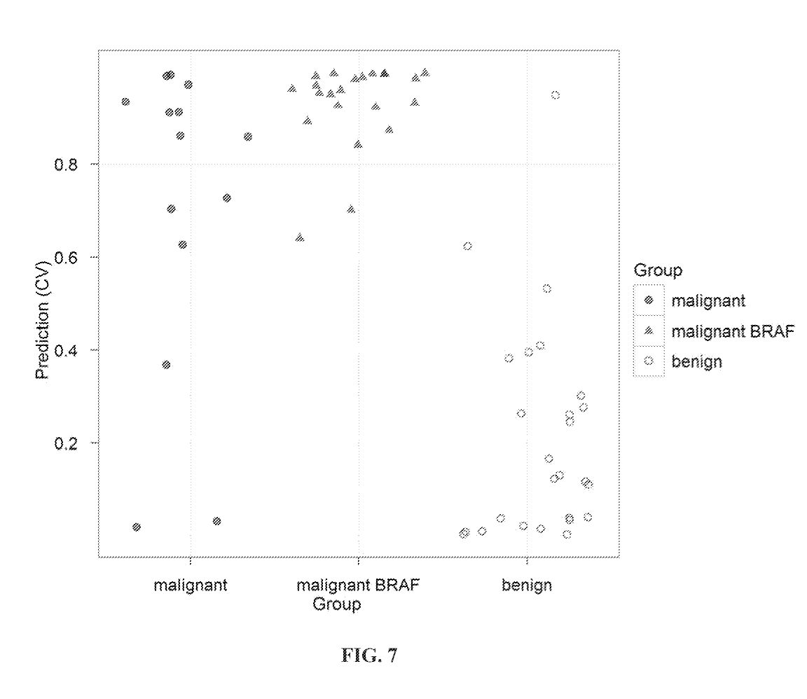

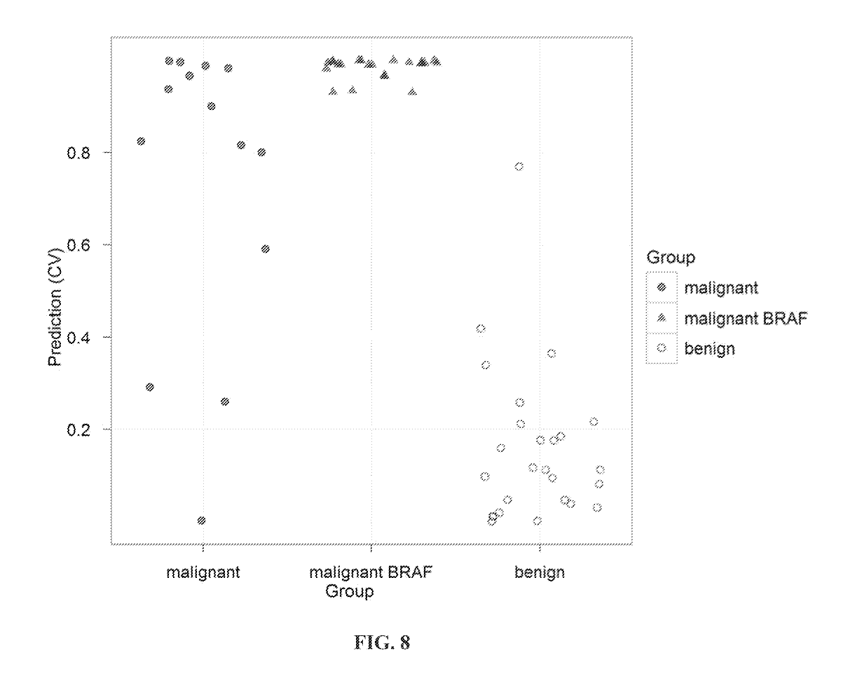

A total of 62 FNAs were also analyzed. RT-qPCR results for each replicate of the 26 miRNA assays are summarized in Table W2. The intercept (w₀) and weight coefficients (wᵢ for i>0) for the constrained logistic regression classifiers trained as described in section entitled “Constrained logistic regression method for constructing miRNA-based classifiers” above are indicated in Table D1. The leave-one-out-cross-validation (LOOCV) estimated accuracies and AUCs of the models were: (1) 5-miRNA model: accuracy 90% (95% CI: 80%-96%), AUC 0.93; (2) 10-miRNA model: accuracy 94% (95% CI: 84%-98%), AUC 0.96. For both the 5- and 10-miRNA models, model sensitivity with regard to BRAF-mutation negative malignant samples was estimated by LOOCV at 79% (95% CI: 49%-95%), while sensitivity for BRAF-mutation positive malignant samples was estimated at 100% (95% CI: 85%-100%). The specificity of (1) the 5-miRNA model was estimated at 88% (95% CI: 70%-98%), while the specificity of (2) the 10-miRNA model was estimated at 96% (95% CI: 80%-100%). The LOOCV classification scores produced by these models are shown in FIG. 7 and FIG. 8, respectively.

TABLE D1

| Predictor | 5 miRNA Model | 10 miRNA Model |
|---|---|---|
| (Intercept) | 4.99 | 3.54 |
| miR-375 | −0.42 | −0.43 |
| miR-221* | | −0.35 |
| miR-222 | −0.09 | −0.33 |
| miR-424 | | −0.27 |
| miR-551b | −0.27 | −0.18 |
| miR-146b-3p | −0.45 | −0.17 |
| miR-31 | | −0.08 |
| miR-139-5p | | 0.42 |
| miR-7 | | 0.51 |
| miR-141 | 1.23 | 0.88 |

The miRNA predictors used by the classifiers break down into two categories according to the sign of their coefficients in the models: those miRs which are substantially overexpressed in malignant samples as compared to benign samples tend to receive strongly negative model coefficients, while those miRs which exhibit similar or lower expression levels in malignant samples as compared with benign enter the models with positive coefficients. The most negatively-weighted miRNA predictor in the 5-miRNA model was found to be miR-146b-3p, with miR-375 a close second (Table D1); for the 10-miRNA model, miR-375 had the largest magnitude coefficient among the negatively weighted miRNAs, with miR-221* coming second. For both the 5- and 10-miRNA models, miR-141 (which showed little difference in expression levels between malignant and benign samples) was assigned the largest positive coefficient.

TABLE X
Patient information and histopathological data for the second set (Set2) of thyroid
FFPE samples.

| | | | | | | | | AJCC/ | |
|---|---|---|---|---|---|---|---|---|---|
| | | | | | | | AJCC/ | UICC | |
| Sample | Pathology- | | | | | Excision | UICC | Stage | miRInform ® |
| ID | (Abbr) | Histopathology | Age | Sex | Ethnicity | Year | Stage | Group | Thyroid |
| AF117 | PTC | Papillary | 38 | F | AA | 1995 | T2N1MX | I | BRAF V600E |
| | | carcinoma | | | | | | | |
| AE118 | FTC | Follicular | 47 | F | C | 1995 | T2NXMX | II | PAX8/PPARg |
| | | carcinoma | | | | | | | |
| AF119 | MNG | Goiter, | 44 | F | C | 1996 | T1bN1MX | III | Neg |
| | | multinodular | | | | | | | |
| AF120 | FTC | Follicular | 46 | F | C | 1996 | T3NXMX | III | Neg |
| | | carcinoma | | | | | | | |
| AF121 | FA | Follicular adenoma | 47 | F | C | 1996 | T2NXMX | II | Neg |
| AF122 | Hash | Follicular Adenoma | 52 | F | C | 2006 | T2NXM0 | II | Neg |
| AF123 | FA | Follicular Adenoma | 41 | F | C | 2007 | T3NXM0 | I | Neg |
| AF124 | NH | Nodular | 54 | M | C | 2009 | N/A | N/A | Neg |
| | | Hyperplasia | | | | | | | |
| AF125 | PTC | Papillary | 24 | M | C | 2007 | T3N1MX | I | BRAF V600E |
| | | Carcinoma | | | | | | | |
| AF126 | PTC | Papillary | 45 | F | C | 2008 | T3NXM0 | III | Neg |
| | | Carcinoma | | | | | | | |
| AF127 | NH | Nodular | 88 | F | C | 2009 | N/A | N/A | Neg |
| | | Hyperplasia | | | | | | | |
| AF128 | FvPTC | Papillary | 38 | F | C | 2009 | T2NXMX | I | Neg |
| | | Carcinoma, | | | | | | | |
| | | Follicular Variant | | | | | | | |
| AF129 | FTC | Follicular | 52 | F | C | 2010 | T2NXM0 | II | Neg |
| | | carcinoma | | | | | | | |
| AF130 | PTC | Papillary | 49 | F | C | 2009 | T3N0M0 | III | Neg |
| | | carcinoma | | | | | | | |
| AF131 | PTC | Papillary | 40 | F | C | 2009 | T2N0M0 | I | Neg |
| | | carcinoma | | | | | | | |
| AF132 | FTC | Follicular | 79 | F | C | 2009 | T1N1M0 | III | Neg |
| | | Carcinoma | | | | | | | |
| AF133 | FA | Hyperplasia | 70 | M | C | 2009 | N/A | N/A | Neg |
| AF134 | PTC | Papillary | 53 | F | C | 2009 | T2N0M0 | II | BRAF V600E |
| | | carcinoma | | | | | | | |
| AF135 | MNG | Goiter, | 73 | M | C | 2009 | N/A | N/A | Neg |
| | | multinodular | | | | | | | |
| AF136 | PTC | Papillary | 77 | M | AP | 2011 | T3NXM0 | III | Neg |
| | | carcinoma | | | | | | | |
| AF137 | PTC | Papillary | 24 | M | AP | 2011 | T3NXM0 | I | BRAF V600E |
| | | carcinoma | | | | | | | |
| AF138 | PTC | Papillary | 19 | F | AP | 2011 | T1bNXM0 | I | BRAF V600E |
| | | carcinoma | | | | | | | |
| AF139 | PTC | Papillary | 67 | F | AP | 2011 | T3NXM0 | III | BRAE V600E |
| | | carcinoma | | | | | | | |
| AF140 | NH | Nodular | 68 | F | C | 2008 | T3NXM0 | III | Neg |
| | | Hyperplasia | | | | | | | |
| AF141 | PTC | Papillary | 58 | F | C | 1994 | T3NXMX | III | BRAF V600E |
| | | carcinoma | | | | | | | |
| AF142 | FA | Follicular adenoma | 36 | M | C | 1994 | T2NXMX | I | Neg |
| AF143 | OFA | Hurthle cell | 52 | M | C | 1994 | T3NXMX | III | Neg |
| | | adenoma | | | | | | | |
| AF144 | FvPTC | Papillary | 29 | F | C | 1994 | T3NXMX | I | BRAF V600E |
| | | carcinoma, | | | | | | | |
| | | follicular variant | | | | | | | |
| AF145 | FA | Follicular adenoma | 38 | F | C | 1994 | N/A | N/A | Neg |
| AF146 | FTC | Follicular | 67 | M | C | 1993 | T3NXMX | III | HRAS Q61R |
| | | carcinoma | | | | | | | |
| AF147 | PTC | Papillary | 54 | F | C | 2008 | T2NXM0 | II | Neg |
| | | Carcinoma | | | | | | | |
| AF148 | NH | nodular | 66 | M | C | 2004 | N/A | N/A | Neg |
| | | hyperplasia | | | | | | | |
| AF149 | NH | Nodular | 45 | M | C | 2004 | N/A | N/A | Neg |
| | | Hyperplasia | | | | | | | |
| AF150 | NH | Nodular | 46 | F | C | 2004 | N/A | N/A | Neg |
| | | Hyperplasia | | | | | | | |
| AF151 | FTC | Follicular | 61 | F | C | 1994 | TXNXMX | | Neg |
| | | carcinoma | | | | | | | |
| AF152 | FA | Follicular adenoma | 65 | F | C | 1995 | N/A | N/A | Neg |
| AF153 | FTC | Follicular | 46 | F | C | 1997 | T2NXMX | | PAX/PPARG |
| | | carcinoma | | | | | | | |
| AF154 | Hash | Hashimoto's | 48 | F | C | 1995 | N/A | N/A | Neg |
| | | thyroiditis | | | | | | | |
| AF156 | NH | Nodular | 29 | F | C | 2004 | T1N0M0 | I | Neg |
| | | Hyperplasia | | | | | | | |
| AF157 | FA | Follicular Adenoma | 47 | M | C | 2006 | T2NXMX | II | Neg |
| AF158 | Hash | Hashimoto's | 81 | M | C | 1995 | T2N0MX | II | Neg |
| | | thyroiditis | | | | | | | |
| AF159 | Hash | Hashimoto's | 45 | F | AP | 1995 | T1aN0MX | I | Neg |
| | | thyroiditis | | | | | | | |
| AF160 | PTC | Papillary | 37 | F | C | 1996 | T2NXMX | I | BRAF V600 |
| | | carcinoma | | | | | | | |
| AF161 | FA | Follicular adenoma | 31 | F | C | 1996 | T2NXMX | I | Neg |
| AF162 | NH | Nodular | N/A | N/A | N/A | N/A | N/A | N/A | Neg |
| | | Hyperplasia | | | | | | | |
| AF163 | FTC | Follicular | 75 | F | BAA | 1994 | T3NXMX | III | NRAS Q61K |
| | | carcinoma | | | | | | | |
| AF164 | FA | Follicular adenoma | 44 | M | C | 1994 | N/A | N/A | Neg |
| AF165 | FTC | Follicular | 60 | F | C | 1995 | T4aNXMX | IVA | Neg |
| | | carcinoma | | | | | | | |
| AF166 | OFA | Hurthle cell | 42 | F | C | 1995 | N/A | N/A | Neg |
| | | adenoma | | | | | | | |
| AF167 | MNG | Goiter, | 46 | F | C | 1995 | N/A | N/A | Neg |
| | | multinodular | | | | | | | |
| AF168 | MNG | Goiter, | 36 | F | C | 1995 | T2NXMX | I | Neg |
| | | multinodular | | | | | | | |
| AF169 | Hash | Hashimoto's | 36 | F | C | 1995 | N/A | N/A | Neg |
| | | thyroiditis | | | | | | | |
| AF170 | FvPTC | Papillary | 35 | M | C | 1997 | T2NXMX | | Neg |
| | | carcinoma, | | | | | | | |
| | | follicular variant | | | | | | | |
| AF171 | FvPTC | Papillary | 60 | F | C | 1997 | T4NXMX | | HRAS Q61L |
| | | carcinoma, | | | | | | | |
| | | follicular variant | | | | | | | |
| AF172 | PTC | Papillary | 52 | F | C | 2005 | T3N1aM0 | III | Neg |
| | | Carcinoma | | | | | | | |
| AF173 | FvPTC | Papillary | 52 | F | C | 2002 | T2NXM0 | II | Neg |
| | | Carcinoma, | | | | | | | |
| | | Follicular Variant | | | | | | | |
| AF174 | PTC | Papillary | N/A | N/A | N/A | N/A | N/A | N/A | BRAF V600E |
| | | Carcinoma | | | | | | | |
| AF176 | PTC | Papillary | 24 | M | C | 1997 | T3N1MX | I | Neg |
| | | carcinoma | | | | | | | |
| AF176 | FvPTC | Papillary | 40 | F | C | 1996 | T2NXMX | I | BRAF V600E |
| | | carcinoma, | | | | | | | |
| | | follicular variant | | | | | | | |
| AF177 | FvPTC | Papillary | 53 | F | C | 1997 | N/A | N/A | Neg |
| | | carcinoma, | | | | | | | |
| | | follicular variant | | | | | | | |
| AF178 | FvPTC | Papillary | 63 | M | C | 2001 | N/A | N/A | BRAF V600E |
| | | Carcinoma, | | | | | | | |
| | | Follicular Variant | | | | | | | |
| AF179 | PTC | Papillary | 52 | F | C | 2006 | T1NXMX | I | Neg |
| | | Carcinoma | | | | | | | |
| AF180 | PTC | Papillary | 46 | F | C | 2006 | T1NXMX | I | Neg |
| | | Adenocarcinoma | | | | | | | |
| AF181 | PTC | Papillary | 62 | F | C | 2006 | T3NXM0 | III | BRAF V600E |
| | | Carcinoma | | | | | | | |
| AF183 | Hash | Hashimoto's | 73 | F | C | 1996 | T1aN0MX | I | neg |
| | | thyroiditis | | | | | | | |

Anaplastic cancer (ATC);
Follicular Adenoma (FA);
oncocytic FA (OFA);
Follicular carcinoma (FTC);
Papillary carcinoma (PTC);
Follicular variant of PTC (FvPTC),
Hashimoto's thyroiditis (HASH);
multinodular goiter (MNG);
hyperplastic nodules (HN);
Age at Excision (Age);
Male (M);
Female (F);
African-American (AA);
Black/African-American (BAA);
Caucasian (C);
Asian/Pacific (AP)

TABLE Y

| | | | Mean | STDEV |
|---|---|---|---|---|
| Sample Name | Mean Benign | STDEV Benign | Malignant | Malignant |
| hsa-miR-150_st | 10.38952 | 1.986007 | 10.6115756 | 1.75562767 |
| hsa-let-7a-2-star_st | 4.357289 | 0.514889 | 4.73780993 | 0.53708965 |
| hsa-let-7a-star_st | 3.46136 | 0.5399 | 3.6352817 | 0.48318111 |
| hsa-miR-513b_st | 10.85016 | 0.938455 | 9.90799044 | 1.85099487 |
| hsa-let-7b-star_st | 7.23489 | 0.583163 | 7.53839807 | 0.84976115 |
| hsa-miR-593-star_st | 9.472863 | 1.171043 | 8.62865919 | 1.48126588 |
| hsa-let-7c-star_st | 5.1015 | 0.479473 | 5.26218298 | 0.57284025 |
| hsa-miR-516a-5p_st | 7.094672 | 0.968459 | 6.3859007 | 1.08219292 |
| hsa-miR-142-5p_st | 5.406472 | 1.857546 | 6.27084979 | 1.47691603 |
| hsa-miR-650_st | 11.0446 | 0.637241 | 10.3854437 | 0.97124484 |
| hsa-miR-513c_st | 8.08382 | 0.957614 | 7.31029183 | 1.34694414 |
| hsa-miR-761_st | 8.196539 | 0.947349 | 7.76052319 | 1.01121665 |
| hsa-let-7f-1-star_st | 5.268617 | 0.542365 | 5.21174304 | 0.61755578 |
| hsa-miR-513a-5p_st | 12.32717 | 0.803544 | 11.6163995 | 1.55772595 |
| hsa-miR-654-5p_st | 11.43621 | 0.805136 | 10.5616472 | 0.97997343 |
| hsa-let-7g-star_st | 4.799798 | 0.37421 | 4.98768358 | 0.47524897 |
| hsa-miR-3163_st | 8.831189 | 0.723743 | 8.2157038 | 0.85874696 |
| hsa-let-7i-star_st | 5.959516 | 0.674877 | 6.93456687 | 0.98886368 |
| hsa-miR-3165_st | 7.767602 | 0.923176 | 7.05148597 | 1.20984278 |
| hsa-miR-3194_st | 11.38148 | 0.707084 | 10.6703154 | 0.90438404 |
| hsa-miR-100-star_st | 3.285287 | 0.574897 | 3.43309333 | 0.62473728 |
| hsa-miR-4251_st | 6.819539 | 0.892629 | 6.26363215 | 1.03574394 |
| hsa-miR-101-star_st | 3.906051 | 0.436347 | 4.3024388 | 0.50828268 |
| hsa-miR-922_st | 6.359004 | 0.788619 | 5.97533821 | 0.95110788 |
| hsa-miR-103-2-star_st | 5.337153 | 0.697506 | 5.74616724 | 0.7137798 |
| hsa-miR-155_st | 9.205806 | 1.316447 | 9.86291669 | 1.24628916 |
| hsa-miR-105_st | 3.953412 | 0.476763 | 3.75434474 | 0.46741637 |
| hsa-miR-127-5p_st | 7.610083 | 0.913334 | 7.12920119 | 1.12004162 |
| hsa-miR-1286_st | 9.388755 | 0.961808 | 9.02744344 | 0.93476374 |
| hsa-miR-106a-star_st | 3.51646 | 0.564608 | 3.62554268 | 0.51885134 |
| hsa-miR-890_st | 6.438965 | 0.98878 | 6.26391341 | 0.98663664 |
| hsa-miR-3126-5p_st | 12.64894 | 0.591335 | 11.7688999 | 1.2446833 |
| hsa-miR-489_st | 9.953939 | 0.730284 | 9.25821725 | 1.4084366 |
| hsa-miR-10a_st | 8.055236 | 1.615627 | 8.94837628 | 1.36028815 |
| hsa-miR-10a-star_st | 5.059565 | 1.029601 | 5.09416832 | 0.71190049 |
| hsa-miR-10b_st | 8.996288 | 1.603747 | 10.2327628 | 1.31631617 |
| hsa-miR-10b-star_st | 4.993942 | 0.695308 | 5.23391564 | 0.63009526 |
| hsa-miR-1178_st | 4.687587 | 0.500872 | 4.45768531 | 0.58350995 |
| hsa-miR-1179_st | 3.37391 | 0.433916 | 3.55336092 | 0.56249274 |
| hsa-miR-1180_st | 9.547859 | 0.541327 | 9.15211729 | 0.45721059 |
| hsa-miR-1181_st | 9.434006 | 0.707851 | 9.14468304 | 0.79915025 |
| hsa-miR-3138_st | 11.2951 | 0.547575 | 10.5022507 | 0.98344812 |
| hsa-miR-220c_st | 6.650857 | 0.82295 | 6.18928893 | 0.74203974 |
| hsa-miR-510_st | 9.297377 | 0.578111 | 8.53620233 | 0.96395687 |
| hsa-miR-1185_st | 3.016118 | 0.63058 | 2.95615293 | 0.55549584 |
| hsa-miR-1193_st | 10.6574 | 0.43507 | 10.3641434 | 0.41480404 |
| hsa-miR-1197_st | 3.724772 | 0.50822 | 3.73017576 | 0.47494647 |
| hsa-miR-1200_st | 4.47716 | 0.433778 | 4.5156683 | 0.45777405 |
| hsa-miR-1201_st | 8.333221 | 1.38038 | 8.86932578 | 1.24780914 |
| hsa-miR-1202_st | 13.77383 | 0.370809 | 13.1939211 | 0.53330107 |
| hsa-miR-30c-1-star_st | 10.55832 | 0.789909 | 9.87482854 | 1.25076796 |
| hsa-miR-1204_st | 5.447949 | 0.591326 | 5.21394368 | 0.64684982 |
| hsa-miR-1205_st | 4.455803 | 0.510428 | 4.29667029 | 0.43260039 |
| hsa-miR-1206_st | 3.136778 | 0.559368 | 3.16466435 | 0.52669384 |
| hsa-miR-1207-3p_st | 4.426257 | 0.509645 | 4.31709345 | 0.41886113 |
| hsa-miR-1207-5p_st | 14.77373 | 0.41229 | 14.3125136 | 0.44885445 |
| hsa-miR-1208_st | 10.91148 | 0.638299 | 10.1011842 | 0.82716953 |
| hsa-miR-622_st | 9.2618 | 0.609418 | 8.72085232 | 1.03477025 |
| hsa-miR-1224-3p_st | 5.545164 | 0.580603 | 5.53508927 | 0.51739318 |
| hsa-miR-2116_st | 6.438526 | 0.857813 | 5.9242753 | 0.87314661 |
| hsa-miR-1225-3p_st | 6.535976 | 0.686195 | 6.36329234 | 0.60803155 |
| hsa-miR-1225-5p_st | 14.59143 | 0.4084 | 14.0737575 | 0.5109831 |
| hsa-miR-1226_st | 5.944648 | 0.659491 | 5.75163868 | 0.48904257 |
| hsa-miR-30b-star_st | 9.3287 | 0.63229 | 8.66928794 | 0.78001447 |
| hsa-miR-1227_st | 4.590626 | 0.691355 | 4.6803536 | 0.60477427 |
| hsa-miR-1295_st | 6.878274 | 0.600932 | 6.35636625 | 0.68770121 |
| hsa-miR-1228-star_st | 15.61034 | 0.573307 | 15.3085406 | 0.5665271 |
| hsa-miR-20b_st | 8.194981 | 1.086889 | 9.64529683 | 1.13784137 |
| hsa-miR-122-star_st | 3.368453 | 0.474343 | 3.40177665 | 0.47901734 |
| hsa-miR-1231_st | 14.53925 | 0.488525 | 14.0000351 | 0.51755774 |
| hsa-miR-1233_st | 6.445083 | 0.633746 | 6.07615059 | 0.57351771 |
| hsa-miR-524-5p_st | 5.412551 | 0.788718 | 5.21779251 | 0.64776897 |
| hsa-miR-1236_st | 6.135532 | 0.73513 | 6.10499204 | 0.67831659 |
| hsa-miR-873_st | 5.434142 | 0.677063 | 5.11675727 | 0.70875546 |
| hsa-miR-1238_st | 7.621242 | 0.721776 | 7.37548619 | 0.6861198 |
| hsa-miR-124_st | 9.061091 | 0.526261 | 8.91111599 | 0.61616007 |
| hsa-miR-1243_st | 4.350738 | 0.443074 | 4.34341673 | 0.5047149 |
| hsa-miR-1244_st | 8.491907 | 0.799179 | 8.61243674 | 1.07054454 |
| hsa-miR-1245_st | 4.318502 | 0.438666 | 4.24588943 | 0.47547173 |
| hsa-miR-3122_st | 12.11072 | 0.396089 | 11.2726089 | 0.86619843 |
| hsa-miR-134_st | 10.21842 | 0.578755 | 9.39273751 | 0.89478316 |
| hsa-miR-1248_st | 10.16793 | 1.127361 | 10.1172362 | 1.60343786 |
| hsa-miR-146a_st | 8.887683 | 1.481866 | 10.5913748 | 1.72802847 |
| hsa-miR-124-star_st | 5.615336 | 0.508358 | 5.37160278 | 0.40689305 |
| hsa-miR-1250_st | 8.384996 | 0.596273 | 7.77720251 | 0.70818305 |
| hsa-miR-764_st | 5.749416 | 0.751104 | 5.43109307 | 0.66064111 |
| hsa-miR-1252_st | 3.215106 | 0.541593 | 3.42039276 | 0.50459537 |
| hsa-miR-223_st | 9.019365 | 1.270088 | 9.99254043 | 1.56807286 |
| hsa-miR-34c-3p_st | 8.568783 | 0.777162 | 7.91659629 | 0.96040607 |
| hsa-miR-1255a_st | 5.237402 | 0.778395 | 5.36918925 | 0.71236113 |
| hsa-miR-1255b_st | 7.983933 | 0.575313 | 7.73166901 | 1.26068293 |
| hsa-miR-1256_st | 3.955908 | 0.462784 | 4.06324904 | 0.52738418 |
| hsa-miR-1257_st | 5.22604 | 0.586825 | 5.24551787 | 0.49267956 |
| hsa-miR-199b-5p_st | 6.969523 | 2.030831 | 6.75422466 | 2.30813172 |
| hsa-miR-1259_st | 3.299872 | 0.535781 | 3.48222159 | 0.5039441 |
| hsa-miR-492_st | 9.328439 | 0.62203 | 8.68643223 | 0.88025514 |
| hsa-miR-3132_st | 9.699389 | 0.581574 | 9.11466067 | 0.98875953 |
| hsa-miR-516b_st | 7.279491 | 0.743569 | 6.7175821 | 0.91554739 |
| hsa-miR-4294_st | 9.088439 | 0.496404 | 8.38722481 | 0.95167841 |
| hsa-miR-125b-2-star_st | 10.12684 | 0.924505 | 9.86394277 | 0.82445524 |
| hsa-miR-126_st | 13.49144 | 1.086588 | 13.9975867 | 0.97182874 |
| hsa-miR-198_st | 12.56028 | 0.476438 | 11.8599303 | 1.01121456 |
| hsa-miR-3191_st | 11.9685 | 0.77925 | 11.4910121 | 1.07972177 |
| hsa-miR-1261_st | 7.937375 | 0.872943 | 7.42052755 | 1.10708599 |
| hsa-miR-1262_st | 4.476336 | 0.662835 | 4.36652798 | 0.52679639 |
| hsa-miR-1263_st | 4.433542 | 0.40919 | 4.54708684 | 0.43130432 |
| hsa-miR-1264_st | 3.614714 | 0.385273 | 3.67854575 | 0.55524343 |
| hsa-miR-1265_st | 5.27597 | 0.383067 | 5.44978149 | 0.5585284 |
| hsa-miR-2114-star_st | 7.402733 | 0.88373 | 6.89938422 | 1.07963391 |
| hsa-miR-1267_st | 4.43422 | 0.444069 | 4.17635775 | 0.38903668 |
| hsa-miR-1268_st | 14.52499 | 0.516985 | 14.0374836 | 0.49493537 |
| hsa-miR-1269_st | 6.005642 | 0.619387 | 5.6353091 | 0.58884571 |
| hsa-miR-509-5p_st | 6.534175 | 0.72875 | 6.07449563 | 0.80518836 |
| hsa-miR-1270_st | 6.653462 | 0.478535 | 6.38125403 | 0.62625484 |
| hsa-miR-1271_st | 7.771615 | 0.646477 | 7.49872364 | 0.77209925 |
| hsa-miR-4303_st | 7.21271 | 0.653178 | 6.71359698 | 0.72842596 |
| hsa-miR-1273_st | 10.14442 | 0.526005 | 9.65652443 | 0.83281863 |
| hsa-miR-583_st | 7.365099 | 0.868159 | 7.12713789 | 1.14313295 |
| hsa-miR-1273d_st | 10.74788 | 0.48916 | 10.4845754 | 0.55836515 |
| hsa-miR-127-3p_st | 6.076071 | 0.846256 | 6.5419768 | 1.49727579 |
| hsa-miR-522-star_st | 6.556959 | 0.734561 | 5.98997852 | 0.60266289 |
| hsa-miR-214-star_st | 5.544811 | 0.861459 | 5.70451843 | 1.13591376 |
| hsa-miR-184_st | 8.438343 | 0.850087 | 7.88354075 | 0.92726689 |
| hsa-miR-614_st | 6.463475 | 0.859388 | 6.14691367 | 0.97929551 |
| hsa-miR-526b_st | 5.889509 | 0.743883 | 5.54332474 | 0.67953542 |
| hsa-miR-1277_st | 3.313252 | 0.613061 | 3.53021114 | 0.54586911 |
| hsa-miR-1278_st | 3.92404 | 0.418186 | 4.1532405 | 0.57555697 |
| hsa-miR-1279_st | 3.547495 | 0.548873 | 3.66349591 | 0.51664879 |
| hsa-miR-409-3p_st | 6.657151 | 0.81253 | 6.5378946 | 1.00805052 |
| hsa-miR-199a-5p_st | 9.697437 | 1.328057 | 9.96347873 | 1.68904486 |
| hsa-miR-1281_st | 10.15523 | 0.660057 | 9.77871988 | 0.60212618 |
| hsa-miR-1282_st | 4.143756 | 0.587692 | 4.06748009 | 0.60470114 |
| hsa-miR-33b-star_st | 7.608316 | 0.469182 | 7.07200218 | 0.6468988 |
| hsa-miR-1284_st | 5.202345 | 0.687226 | 5.15836567 | 0.45722521 |
| hsa-miR-1253_st | 5.143236 | 0.680207 | 4.99361874 | 0.61031078 |
| hsa-miR-584_st | 8.04233 | 0.610655 | 7.44698406 | 0.78758502 |
| hsa-miR-1287_st | 6.330628 | 0.567947 | 6.32517179 | 0.54468857 |
| hsa-miR-15a_st | 9.400582 | 1.252219 | 11.2912451 | 1.16714496 |
| hsa-miR-1289_st | 4.784908 | 0.507687 | 4.70509184 | 0.48657725 |
| hsa-miR-1290_st | 10.6555 | 0.508518 | 10.688131 | 0.79078482 |
| hsa-miR-2117_st | 4.350308 | 0.547362 | 4.04519566 | 0.56664069 |
| hsa-miR-1292_st | 13.0471 | 0.45811 | 12.435359 | 0.63904949 |
| hsa-miR-1293_st | 7.765086 | 0.515708 | 7.25481822 | 0.58041738 |
| hsa-miR-129-3p_st | 3.9061 | 0.56408 | 4.28944547 | 1.08009037 |
| hsa-miR-520d-5p_st | 7.202907 | 0.777321 | 6.78993349 | 0.76731494 |
| hsa-miR-526a_st | 6.923736 | 0.717346 | 6.62565189 | 0.7583461 |
| hsa-miR-129-5p_st | 11.93347 | 0.497136 | 11.2705039 | 0.72035409 |
| hsa-miR-1254_st | 12.26813 | 0.629792 | 11.56637 | 0.83408274 |
| hsa-miR-1297_st | 3.238424 | 0.452585 | 3.22577271 | 0.60188484 |
| hsa-miR-1298_st | 4.943392 | 0.710928 | 4.99876047 | 0.7316201 |
| hsa-miR-1299_st | 5.782281 | 0.6536 | 5.63159734 | 0.98084374 |
| hsa-miR-129-star_st | 4.372468 | 0.568782 | 4.50913149 | 0.54666149 |
| hsa-miR-1301_st | 8.801853 | 0.445676 | 8.50042486 | 0.45196955 |
| hsa-miR-19b_st | 9.19229 | 1.308257 | 10.5795935 | 1.2774744 |
| hsa-miR-1303_st | 10.67638 | 0.533078 | 10.2721073 | 0.68711508 |
| hsa-miR-1304_st | 7.661559 | 0.74942 | 7.34902996 | 0.93193949 |
| hsa-miR-1305_st | 4.248962 | 0.580281 | 4.1858747 | 0.47206909 |
| hsa-miR-550_st | 12.18404 | 0.629339 | 11.6879493 | 0.8199054 |
| hsa-miR-1307_st | 14.08975 | 0.586589 | 13.5355464 | 0.57386276 |
| hsa-miR-1308_st | 14.94022 | 0.605185 | 15.2503915 | 0.55230295 |
| hsa-miR-4291_st | 7.029601 | 0.991374 | 7.7994051 | 0.94926531 |
| hsa-miR-130a-star_st | 3.978493 | 0.488964 | 3.98471736 | 0.46117242 |
| hsa-miR-130b_st | 8.085583 | 0.751431 | 8.50271914 | 0.81869108 |
| hsa-miR-130b-star_st | 4.852718 | 0.663151 | 5.12802279 | 0.74686133 |
| hsa-miR-921_st | 12.04582 | 0.530387 | 11.37426 | 0.82357838 |
| hsa-miR-520a-5p_st | 5.059042 | 0.608316 | 4.79466106 | 0.53072223 |
| hsa-miR-711_st | 12.27524 | 0.589288 | 11.7096369 | 0.78223308 |
| hsa-miR-1323_st | 8.829832 | 0.603626 | 8.18944317 | 0.71589806 |
| hsa-miR-1324_st | 3.571799 | 0.533497 | 3.70749581 | 0.60082365 |
| hsa-miR-490-3p_st | 6.38212 | 0.659382 | 5.94635026 | 0.6740777 |
| hsa-miR-718_st | 9.107095 | 0.612011 | 8.50757811 | 0.91841826 |
| hsa-miR-133b_st | 5.690387 | 1.252192 | 6.10093928 | 2.20827788 |
| hsa-miR-431-star_st | 6.962108 | 0.596349 | 6.27230418 | 0.69489517 |
| hsa-miR-552_st | 6.013338 | 0.719863 | 5.56340615 | 0.6974508 |
| hsa-miR-28-3p_st | 7.126921 | 1.096338 | 7.92138802 | 1.10127288 |
| hsa-miR-4265_st | 8.251232 | 0.647082 | 7.54002814 | 0.72307606 |
| hsa-miR-643_st | 4.276702 | 0.678278 | 4.01396145 | 0.49684553 |
| hsa-miR-136_st | 3.090885 | 0.640674 | 3.23295897 | 0.58806563 |
| hsa-miR-136-star_st | 3.280648 | 0.527551 | 3.38422196 | 0.55529244 |
| hsa-miR-137_st | 3.480772 | 0.387736 | 3.53007945 | 0.47842398 |
| hsa-miR-138_st | 10.59837 | 1.100913 | 10.3596559 | 1.29577727 |
| hsa-miR-138-1-star_st | 8.597092 | 0.518887 | 8.15914785 | 0.72851942 |
| hsa-miR-138-2-star_st | 5.306019 | 0.416304 | 5.39000029 | 0.540153 |
| hsa-miR-139-3p_st | 8.310386 | 0.662357 | 7.93959998 | 0.65140966 |
| hsa-miR-139-5p_st | 7.9866 | 1.235272 | 7.89525401 | 1.27891605 |
| hsa-miR-140-3p_st | 10.74996 | 0.551884 | 11.0888308 | 0.66934848 |
| hsa-miR-140-5p_st | 5.713316 | 1.0075 | 6.6856171 | 1.21515569 |
| hsa-miR-381_st | 6.47366 | 0.789362 | 6.21027434 | 0.92935905 |
| hsa-miR-141-star_st | 3.756301 | 0.425378 | 4.29696272 | 0.84231106 |
| hsa-miR-17-star_st | 6.15162 | 0.608628 | 7.10145886 | 0.91817935 |
| hsa-miR-4300_st | 8.291227 | 0.742608 | 7.79887903 | 0.92079282 |
| hsa-miR-143_st | 11.98463 | 1.07468 | 12.5033545 | 1.12331495 |
| hsa-miR-143-star_st | 6.366789 | 0.764475 | 6.93224596 | 0.94194012 |
| hsa-miR-144_st | 3.478006 | 0.469105 | 3.44911308 | 0.57199178 |
| hsa-miR-3198_st | 10.25799 | 0.399259 | 9.6473457 | 0.70473787 |
| hsa-miR-145_st | 13.02634 | 1.036508 | 13.2760415 | 1.16455684 |
| hsa-miR-145-star_st | 5.576553 | 1.195807 | 6.09930048 | 1.18613508 |
| hsa-miR-1468_st | 6.041576 | 0.706105 | 5.96189889 | 0.72289541 |
| hsa-miR-1469_st | 15.85414 | 0.655596 | 15.7558719 | 0.61472764 |
| hsa-miR-1973_st | 14.84394 | 0.914458 | 14.2257825 | 1.01519972 |
| hsa-miR-3154_st | 11.841 | 0.394263 | 11.2308784 | 0.63736609 |
| hsa-miR-148a-star_st | 5.335065 | 0.856261 | 5.48777864 | 0.74163478 |
| hsa-miR-92a-1-star_st | 8.43774 | 0.627976 | 7.8116457 | 0.67334339 |
| hsa-miR-147_st | 4.369728 | 0.460573 | 4.22191724 | 0.45406229 |
| hsa-miR-194-star_st | 9.014305 | 0.636976 | 8.37899601 | 0.94644073 |
| hsa-miR-1471_st | 11.1678 | 0.665485 | 10.4022786 | 0.88944467 |
| hsa-miR-147b_st | 4.710862 | 0.539004 | 4.66154276 | 0.35880491 |
| hsa-miR-148a_st | 10.876 | 1.381937 | 11.3467232 | 1.35379965 |
| hsa-miR-4299_st | 13.56937 | 0.715054 | 12.9855045 | 1.29560853 |
| hsa-miR-135a-star_st | 13.41044 | 0.452887 | 12.72747 | 0.7998393 |
| hsa-miR-612_st | 12.27801 | 0.527558 | 11.6363967 | 0.75378182 |
| hsa-miR-1321_st | 7.800694 | 0.625883 | 7.3046568 | 0.82545588 |
| hsa-miR-149-star_st | 15.7825 | 0.579601 | 15.539387 | 0.50116132 |
| hsa-miR-604_st | 9.410595 | 0.601619 | 8.98749498 | 1.09741636 |
| hsa-miR-150-star_st | 14.39042 | 0.397546 | 13.8702233 | 0.56661051 |
| hsa-miR-125b-1-star_st | 12.03885 | 0.510609 | 11.5147687 | 0.68349007 |
| hsa-miR-1183_st | 10.46254 | 0.457036 | 9.91407393 | 0.64947921 |
| hsa-miR-152_st | 9.983069 | 1.113642 | 10.1956863 | 1.05953698 |
| hsa-miR-153_st | 3.753755 | 0.499191 | 3.90025313 | 0.67509779 |
| hsa-miR-1537_st | 3.544254 | 0.482314 | 3.76585847 | 0.4795435 |
| hsa-miR-1538_st | 8.138589 | 0.474216 | 7.83350787 | 0.64922918 |
| hsa-miR-1539_st | 5.873557 | 0.553469 | 5.8219744 | 0.50308612 |
| hsa-miR-154_st | 4.757826 | 0.490487 | 4.64627724 | 0.96069642 |
| hsa-miR-154-star_st | 4.969782 | 0.736718 | 4.76407291 | 0.89497382 |
| hsa-miR-432-star_st | 5.0538 | 0.649495 | 4.96343474 | 0.56447033 |
| hsa-miR-155-star_st | 3.68138 | 0.563039 | 3.7271668 | 0.51374138 |
| hsa-miR-298_st | 11.08164 | 0.553009 | 10.5243045 | 0.83832428 |
| hsa-miR-15a-star_st | 5.04323 | 0.639444 | 4.66767205 | 0.46634104 |
| hsa-miR-2110_st | 11.20791 | 0.659696 | 10.6376019 | 0.73598132 |
| hsa-miR-15b-star_st | 3.861323 | 0.520095 | 3.82552288 | 0.35127867 |
| hsa-miR-1266_st | 9.871099 | 0.519955 | 9.21495988 | 0.66398447 |
| hsa-miR-16-1-star_st | 4.506678 | 0.417781 | 4.67082683 | 0.45838599 |
| hsa-miR-16-2-star_st | 4.587863 | 0.582322 | 4.96267077 | 0.81359521 |
| hsa-miR-490-5p_st | 7.772704 | 0.712981 | 7.24444026 | 0.72053352 |
| hsa-miR-19a_st | 5.362558 | 1.205284 | 6.68591444 | 1.36376854 |
| hsa-miR-3192_st | 9.269805 | 0.523768 | 8.65854263 | 0.78246497 |
| hsa-miR-3155_st | 8.0444 | 0.520427 | 7.68563039 | 0.6627022 |
| hsa-miR-187-star_st | 11.4036 | 0.38155 | 10.6131607 | 0.58284327 |
| hsa-miR-16_st | 12.9577 | 0.874914 | 14.1451377 | 0.91414373 |
| hsa-miR-936_st | 11.30008 | 0.425683 | 10.6749869 | 0.57387014 |
| hsa-miR-181c-star_st | 7.423511 | 0.700312 | 7.27320662 | 0.72460569 |
| hsa-miR-379_st | 5.900505 | 0.673361 | 5.82248801 | 0.87968533 |
| hsa-miR-370_st | 11.52791 | 0.552752 | 10.8287245 | 0.67196726 |
| hsa-miR-541_st | 8.817298 | 0.49997 | 8.27813082 | 0.83987041 |
| hsa-miR-518c-star_st | 8.815813 | 0.483748 | 8.25369724 | 0.85647824 |
| hsa-miR-487b_st | 8.545791 | 0.924969 | 9.93495987 | 0.91614829 |
| hsa-miR-182-star_st | 3.526463 | 0.437325 | 3.69208954 | 0.5064464 |
| hsa-miR-3121_st | 8.045173 | 0.825329 | 7.69154438 | 1.0003751 |
| hsa-miR-3190-5p_st | 10.57986 | 0.459501 | 9.94191873 | 0.76952677 |
| hsa-miR-1203_st | 9.303836 | 0.548117 | 8.72714881 | 0.71067448 |
| hsa-miR-185_st | 9.233069 | 0.703361 | 9.77309887 | 0.87678544 |
| hsa-miR-185-star_st | 9.818401 | 0.625494 | 9.256766 | 0.8406816 |
| hsa-miR-4317_st | 10.08478 | 0.647379 | 9.65929362 | 0.61370698 |
| hsa-miR-186-star_st | 3.151068 | 0.536852 | 3.16501739 | 0.49804634 |
| hsa-miR-187_st | 5.410671 | 0.694775 | 5.6881569 | 1.08404849 |
| hsa-miR-4257_st | 12.73521 | 0.380841 | 12.0858235 | 0.69964238 |
| hsa-miR-188-3p_st | 4.753711 | 0.544941 | 4.91175606 | 0.50471447 |
| hsa-miR-1184_st | 5.223914 | 0.594474 | 5.13021174 | 0.49863843 |
| hsa-miR-18a_st | 4.652306 | 0.624515 | 5.34774058 | 0.69489563 |
| hsa-miR-18a-star_st | 5.279843 | 0.672291 | 5.22757857 | 0.58618275 |
| hsa-miR-3140_st | 5.429276 | 0.586338 | 5.25824083 | 0.56121471 |
| hsa-miR-18b-star_st | 4.556296 | 0.43412 | 4.49602146 | 0.56800245 |
| hsa-miR-190_st | 3.000131 | 0.684851 | 2.90799003 | 0.67836777 |
| hsa-miR-601_st | 6.55051 | 0.728232 | 5.98558272 | 0.6274005 |
| hsa-miR-1909_st | 14.91129 | 0.466363 | 14.523262 | 0.53579258 |
| hsa-miR-342-5p_st | 5.824628 | 0.767692 | 6.33479494 | 1.08680023 |
| hsa-miR-575_st | 13.71488 | 0.571579 | 13.0556872 | 0.84194687 |
| hsa-miR-885-5p_st | 7.082986 | 0.705171 | 6.63001186 | 0.82935358 |
| hsa-miR-300_st | 6.09385 | 0.657001 | 5.72401992 | 0.71914145 |
| hsa-miR-1911_st | 4.43363 | 0.595786 | 4.35282976 | 0.42551039 |
| hsa-miR-1911-star_st | 5.474874 | 0.658405 | 5.44707453 | 0.48219626 |
| hsa-miR-1912_st | 4.756903 | 0.401043 | 4.74886851 | 0.44521076 |
| hsa-miR-4266_st | 7.058794 | 0.622565 | 6.5613542 | 0.69611989 |
| hsa-miR-1914_st | 5.933091 | 0.472271 | 5.98994525 | 0.6213241 |
| hsa-miR-3190-3p_st | 11.26445 | 0.391117 | 10.5551338 | 0.69587402 |
| hsa-miR-618_st | 4.056529 | 0.529376 | 4.09823745 | 0.55298877 |
| hsa-miR-1276_st | 7.628428 | 0.732648 | 6.95097618 | 0.76855772 |
| hsa-miR-191-star_st | 6.877947 | 0.797432 | 6.65568204 | 0.76292551 |
| hsa-miR-1258_st | 5.336432 | 0.65674 | 4.98843541 | 0.48470053 |
| hsa-miR-192-star_st | 3.90578 | 0.444768 | 4.14022609 | 0.49228908 |
| hsa-miR-193a-3p_st | 7.54646 | 1.039906 | 7.85222602 | 1.05332127 |
| hsa-miR-193a-5p_st | 13.13197 | 0.543668 | 12.5398464 | 0.52087754 |
| hsa-miR-3157_st | 5.510556 | 0.397314 | 5.10961723 | 0.49942506 |
| hsa-miR-193b-star_st | 11.46221 | 0.522768 | 10.9791069 | 0.52186699 |
| hsa-miR-194_st | 6.856921 | 1.382171 | 8.34760954 | 1.31545108 |
| hsa-miR-4307_st | 3.894476 | 0.569672 | 3.85085686 | 0.59324324 |
| hsa-miR-195_st | 11.21602 | 1.049426 | 11.5275964 | 1.36506121 |
| hsa-miR-610_st | 5.905776 | 0.546873 | 5.68048794 | 0.57907008 |
| hsa-miR-196a_st | 4.516189 | 0.636584 | 4.59412885 | 1.00047501 |
| hsa-miR-196a-star_st | 5.924024 | 0.840354 | 5.53443168 | 1.00990487 |
| hsa-miR-196b_st | 4.102701 | 0.485449 | 4.04742534 | 0.59084418 |
| hsa-miR-196b-star_st | 5.907676 | 0.554815 | 5.73416715 | 0.63995327 |
| hsa-let-7f-2-star_st | 3.876969 | 0.497875 | 4.02648693 | 0.41233585 |
| hsa-miR-1972_st | 12.5182 | 0.513316 | 12.1847632 | 0.5538262 |
| hsa-miR-3184_st | 10.57034 | 0.620189 | 9.96773608 | 0.8452033 |
| hsa-miR-523-star_st | 6.608064 | 0.792583 | 6.14320694 | 0.69851611 |
| hsa-miR-1976_st | 6.908549 | 0.785032 | 6.68599813 | 0.86808183 |
| hsa-miR-625-star_st | 4.640855 | 0.638232 | 4.85998914 | 0.66493379 |
| hsa-miR-587_st | 5.08626 | 0.451682 | 4.69517571 | 0.58532861 |
| hsa-miR-199a-3p_st | 11.09522 | 1.29475 | 11.2147133 | 1.84542999 |
| hsa-miR-1910_st | 9.12995 | 0.525638 | 8.6751606 | 0.62777045 |
| hsa-miR-199b-3p_st | 11.18733 | 1.253928 | 11.3040812 | 1.86805297 |
| hsa-miR-639_st | 9.430837 | 0.737202 | 8.80604054 | 0.64250866 |
| hsa-miR-1909-star_st | 8.852319 | 0.527047 | 8.6380658 | 0.57762219 |
| hsa-miR-19a-star_st | 3.248277 | 0.570135 | 3.25951135 | 0.53384864 |
| hsa-miR-29c-star_st | 7.412177 | 0.987097 | 8.52049852 | 0.88546126 |
| hsa-miR-940_st | 9.142737 | 0.483829 | 8.60968464 | 0.57783102 |
| hsa-miR-19b-2-star_st | 3.159456 | 0.518061 | 3.09772114 | 0.51887323 |
| hsa-miR-4269_st | 8.759823 | 0.448679 | 8.30601513 | 0.76409129 |
| hsa-miR-200a-star_st | 6.786583 | 0.681635 | 7.05532548 | 0.81557213 |
| hsa-miR-1182_st | 10.83303 | 0.373567 | 10.1913771 | 0.70060365 |
| hsa-miR-518f-star_st | 6.904936 | 0.546295 | 6.67131476 | 0.61257083 |
| hsa-miR-92a-2-star_st | 9.340087 | 0.559116 | 8.82478712 | 0.9843132 |
| hsa-miR-200c-star_st | 4.285809 | 0.539298 | 4.49389333 | 0.53785296 |
| hsa-miR-202_st | 9.494886 | 0.507772 | 9.00321838 | 0.60938362 |
| hsa-miR-202-star_st | 2.994445 | 0.594615 | 3.09279121 | 0.66759881 |
| hsa-miR-1914-star_st | 13.47372 | 0.472301 | 12.9179855 | 0.65927069 |
| hsa-miR-204_st | 7.90583 | 2.102357 | 6.50651726 | 2.25679341 |
| hsa-miR-665_st | 13.09387 | 0.396703 | 12.651798 | 0.66511811 |
| hsa-miR-2052_st | 3.011014 | 0.673542 | 3.21956118 | 0.60559582 |
| hsa-miR-2053_st | 3.642616 | 0.587171 | 3.71470981 | 0.47222552 |
| hsa-miR-2054_st | 2.782261 | 0.843189 | 2.81005144 | 0.72992988 |
| hsa-miR-205-star_st | 5.567501 | 0.629178 | 5.5937037 | 0.68009279 |
| hsa-miR-206_st | 6.360138 | 0.714237 | 6.45495418 | 1.56255028 |
| hsa-miR-208a_st | 3.988709 | 0.591335 | 4.04108824 | 0.62465443 |
| hsa-miR-208b_st | 3.840507 | 0.564059 | 4.0074051 | 0.38386192 |
| hsa-miR-18b_st | 4.317941 | 0.399829 | 4.75299489 | 0.59182495 |
| hsa-miR-20a-star_st | 4.08669 | 0.761749 | 4.82009614 | 1.17644656 |
| hsa-miR-3181_st | 9.048286 | 0.551977 | 8.40383643 | 0.74854306 |
| hsa-miR-615-5p_st | 11.51163 | 0.63811 | 11.0233904 | 0.85555952 |
| hsa-miR-4259_st | 11.24691 | 0.429476 | 10.5886901 | 0.53970714 |
| hsa-miR-210_st | 13.14354 | 0.533177 | 12.5272017 | 0.55018594 |
| hsa-miR-211_st | 5.324104 | 0.699883 | 4.88568036 | 0.52373053 |
| hsa-miR-887_st | 9.677032 | 0.43282 | 9.1766726 | 0.61310955 |
| hsa-miR-2113_st | 5.1192 | 0.786091 | 5.07041276 | 0.46627215 |
| hsa-miR-2114_st | 4.522777 | 0.619019 | 4.27711166 | 0.53249886 |
| hsa-miR-943_st | 7.676862 | 0.632179 | 7.14169936 | 0.65822836 |
| hsa-miR-2115_st | 3.29828 | 0.496781 | 3.5167075 | 0.62811412 |
| hsa-miR-2115-star_st | 10.74665 | 0.768738 | 10.4071738 | 0.90301985 |
| hsa-miR-183-star_st | 7.578354 | 0.601006 | 7.25041207 | 0.5974665 |
| hsa-miR-2116-star_st | 4.382764 | 0.611014 | 4.43305146 | 0.48331611 |
| hsa-miR-920_st | 10.72006 | 0.547491 | 10.161402 | 0.73787489 |
| hsa-miR-212_st | 6.381518 | 0.551417 | 6.7216838 | 0.55735742 |
| hsa-miR-214_st | 10.74818 | 0.507169 | 10.578116 | 0.71466936 |
| hsa-miR-1825_st | 7.046347 | 0.568985 | 6.82127986 | 0.67715213 |
| hsa-miR-215_st | 5.718463 | 1.069816 | 6.18895156 | 1.29157609 |
| hsa-miR-216a_st | 3.83513 | 0.353766 | 3.77428564 | 0.5446599 |
| hsa-miR-195-star_st | 5.630064 | 0.658875 | 5.60610538 | 0.63156415 |
| hsa-miR-217_st | 3.462462 | 0.434731 | 3.63432158 | 0.49269356 |
| hsa-miR-23a-star_st | 11.11531 | 0.466606 | 10.6867179 | 0.66917242 |
| hsa-miR-25-star_st | 12.97714 | 0.466602 | 12.4256332 | 0.74302521 |
| hsa-miR-218-2-star_st | 4.510064 | 0.583866 | 4.33482032 | 0.54264988 |
| hsa-miR-1273c_st | 10.87996 | 0.501208 | 10.4793145 | 0.82767498 |
| hsa-miR-219-2-3p_st | 7.345337 | 0.452678 | 6.94335392 | 0.604779 |
| hsa-miR-637_st | 12.37203 | 0.483499 | 11.7189133 | 0.66713904 |
| hsa-miR-21-star_st | 6.666717 | 0.820662 | 7.26837139 | 0.91925052 |
| hsa-miR-22_st | 11.39475 | 1.209008 | 12.1907829 | 0.84367058 |
| hsa-miR-220a_st | 3.41554 | 0.391169 | 3.66550572 | 0.53817747 |
| hsa-miR-220b_st | 4.009127 | 0.536872 | 4.25943623 | 0.48269638 |
| hsa-miR-219-1-3p_st | 5.880131 | 0.41131 | 5.59820794 | 0.53405259 |
| hsa-miR-4316_st | 7.811901 | 0.539669 | 7.33102376 | 0.68199014 |
| hsa-miR-877_st | 12.68284 | 0.404216 | 12.0653331 | 0.57820531 |
| hsa-miR-636_st | 7.92034 | 0.645947 | 7.42920457 | 0.81254252 |
| hsa-miR-3177_st | 12.39135 | 0.469506 | 11.7334651 | 0.61293037 |
| hsa-miR-1249_st | 5.192321 | 0.650324 | 5.14602263 | 0.70941235 |
| hsa-miR-223-star_st | 4.107594 | 0.541261 | 4.50939324 | 0.53768259 |
| hsa-miR-1224-5p_st | 13.77368 | 0.329311 | 13.1571558 | 0.52064089 |
| hsa-miR-564_st | 12.58704 | 0.366432 | 12.0217239 | 0.68890318 |
| hsa-miR-2276_st | 11.62496 | 0.557122 | 10.9767434 | 0.69654964 |
| hsa-miR-2277_st | 7.508897 | 0.530097 | 7.15621848 | 0.71560615 |
| hsa-miR-2278_st | 6.574273 | 0.592552 | 6.31242414 | 0.58854562 |
| hsa-miR-22-star_st | 7.319068 | 1.459441 | 8.38139104 | 1.22092202 |
| hsa-miR-2355_st | 4.45229 | 0.547746 | 4.35902824 | 0.49593076 |
| hsa-miR-769-3p_st | 9.79921 | 0.731407 | 9.30951524 | 0.87187279 |
| hsa-miR-1913_st | 7.906398 | 0.52029 | 7.5443767 | 0.56587509 |
| hsa-miR-1237_st | 7.433565 | 0.586333 | 7.158132 | 0.77084947 |
| hsa-miR-1275_st | 13.80187 | 0.308546 | 13.3347106 | 0.6789055 |
| hsa-miR-30a_st | 12.24781 | 1.042277 | 13.2422955 | 0.99546162 |
| hsa-miR-20b-star_st | 5.93051 | 0.516315 | 5.65993576 | 0.55367313 |
| hsa-miR-24-2-star_st | 6.10863 | 0.959849 | 7.20035511 | 1.00686568 |
| hsa-miR-3173_st | 10.14315 | 0.452426 | 9.59281524 | 0.74737154 |
| hsa-miR-3148_st | 5.688379 | 0.528645 | 5.44543494 | 0.60379501 |
| hsa-miR-4271_st | 12.84732 | 0.335384 | 12.2097731 | 0.6799478 |
| hsa-miR-659_st | 11.09031 | 0.456494 | 10.5973179 | 0.70208839 |
| hsa-miR-26a-2-star_st | 2.926497 | 0.684608 | 3.07198249 | 0.52073345 |
| hsa-miR-1228_st | 7.447184 | 0.687557 | 6.97441665 | 0.6510955 |
| hsa-miR-26b-star_st | 5.295597 | 0.59454 | 6.16374651 | 0.90640347 |
| hsa-miR-3174_st | 7.538033 | 0.791219 | 7.37725199 | 0.73305347 |
| hsa-miR-27a-star_st | 5.74851 | 0.651479 | 5.85742523 | 0.78373376 |
| hsa-miR-1285_st | 12.8171 | 0.377748 | 12.4579705 | 0.64937845 |
| hsa-miR-483-5p_st | 12.11021 | 0.389285 | 11.6618691 | 0.67438196 |
| hsa-miR-1306_st | 7.226534 | 0.437341 | 6.86211736 | 0.55081767 |
| hsa-miR-431_st | 4.179814 | 0.517082 | 4.50994212 | 0.98337035 |
| hsa-miR-765_st | 9.411896 | 0.416691 | 8.99438853 | 0.72723581 |
| hsa-miR-2909_st | 4.051542 | 0.434154 | 4.25558825 | 0.47427972 |
| hsa-miR-296-3p_st | 11.66792 | 0.473418 | 11.1382243 | 0.5810927 |
| hsa-miR-296-5p_st | 4.748007 | 0.603437 | 4.95345489 | 0.77199839 |
| hsa-miR-297_st | 5.275717 | 0.681227 | 5.04247384 | 0.66100959 |
| hsa-miR-323b-5p_st | 7.656137 | 0.597688 | 7.49801768 | 0.64594115 |
| hsa-miR-299-3p_st | 7.60544 | 0.80098 | 7.39980863 | 0.7412039 |
| hsa-miR-299-5p_st | 4.187871 | 0.437855 | 4.28062801 | 0.9515708 |
| hsa-miR-487a_st | 5.905714 | 0.64278 | 6.26788572 | 0.50311898 |
| hsa-miR-29a-star_st | 3.587107 | 0.518688 | 3.62520467 | 0.61848445 |
| hsa-miR-625_st | 7.005339 | 0.586543 | 7.14900401 | 0.65749234 |
| hsa-miR-611_st | 8.444941 | 0.7318 | 8.09734071 | 0.83860473 |
| hsa-miR-3186-3p_st | 9.128762 | 0.536675 | 9.1468968 | 0.69633903 |
| hsa-miR-1294_st | 5.949484 | 0.459751 | 5.70786726 | 0.52188169 |
| hsa-miR-3147_st | 10.95659 | 0.430452 | 10.3402127 | 0.51976672 |
| hsa-miR-4285_st | 11.98503 | 0.626573 | 11.3162171 | 0.85536076 |
| hsa-miR-125a-3p_st | 11.87545 | 0.416548 | 11.5371485 | 0.47433146 |
| hsa-miR-301b_st | 3.380898 | 0.511299 | 3.5384237 | 0.60934524 |
| hsa-miR-302a_st | 3.487983 | 0.500219 | 3.53226882 | 0.51636224 |
| hsa-miR-518e-star_st | 6.871526 | 0.721917 | 6.55741357 | 0.62754693 |
| hsa-miR-302b_st | 3.097469 | 0.719737 | 3.13952956 | 0.66636298 |
| hsa-miR-302b-star_st | 4.473941 | 0.468379 | 4.28003453 | 0.57199119 |
| hsa-miR-302c_st | 3.091218 | 0.528866 | 3.07109404 | 0.56695864 |
| hsa-miR-302c-star_st | 9.322559 | 0.520556 | 8.75764794 | 0.79378319 |
| hsa-miR-302d_st | 3.310685 | 0.540234 | 3.27178825 | 0.49772924 |
| hsa-miR-302d-star_st | 8.81876 | 0.657124 | 8.54582522 | 0.83334835 |
| hsa-miR-302e_st | 3.483191 | 0.55328 | 3.62056265 | 0.47252215 |
| hsa-miR-302f_st | 3.291869 | 0.558694 | 3.4660946 | 0.53620394 |
| hsa-miR-3065-3p_st | 5.198465 | 0.766017 | 5.2761636 | 0.86992375 |
| hsa-miR-3130-5p_st | 7.295173 | 0.751871 | 6.99377791 | 0.64479453 |
| hsa-miR-3074_st | 5.238918 | 0.519661 | 5.09982756 | 0.41671329 |
| hsa-miR-1226-star_st | 12.10807 | 0.441625 | 11.5540232 | 0.445895 |
| hsa-miR-3180-5p_st | 7.029548 | 0.587666 | 6.66773966 | 0.63189876 |
| hsa-miR-1229_st | 7.024116 | 0.46561 | 7.38720818 | 0.57396368 |
| hsa-miR-1272_st | 4.695851 | 0.559417 | 4.49762795 | 0.48101175 |
| hsa-miR-30c_st | 13.35955 | 1.077312 | 14.0569402 | 0.95764945 |
| hsa-miR-675_st | 13.71378 | 0.543176 | 13.2156948 | 0.67167374 |
| hsa-miR-30c-2-star_st | 7.538879 | 0.653015 | 7.44417634 | 0.70232822 |
| hsa-miR-1827_st | 4.679071 | 0.530464 | 4.90573725 | 0.515662 |
| hsa-miR-30d-star_st | 3.72744 | 0.535907 | 4.02268804 | 0.66152951 |
| hsa-miR-30e_st | 9.577361 | 1.245257 | 10.6078373 | 1.2364498 |
| hsa-miR-1915-star_st | 7.190495 | 0.680844 | 7.02434269 | 0.77579649 |
| hsa-miR-1322_st | 4.56829 | 0.45671 | 4.42512905 | 0.52052645 |
| hsa-miR-3115_st | 3.632377 | 0.447203 | 3.79449863 | 0.52190118 |
| hsa-miR-3116_st | 7.160553 | 0.809077 | 6.82579759 | 1.01804093 |
| hsa-miR-3117_st | 4.727633 | 0.447175 | 4.61074147 | 0.60607755 |
| hsa-miR-3118_st | 3.733644 | 0.498701 | 3.87302011 | 0.49055634 |
| hsa-miR-519b-5p_st | 6.371297 | 0.620707 | 6.10313752 | 0.63292002 |
| hsa-miR-3120_st | 6.791567 | 0.715292 | 6.44490673 | 0.73884454 |
| hsa-miR-520d-3p_st | 5.592855 | 0.542073 | 5.38920015 | 0.47207332 |
| hsa-miR-326_st | 6.013079 | 0.451521 | 6.05083719 | 0.54322182 |
| hsa-miR-3123_st | 2.914904 | 0.638766 | 2.9945243 | 0.58600991 |
| hsa-miR-3124_st | 12.72199 | 0.511229 | 12.2259446 | 0.62681791 |
| hsa-miR-3125_st | 7.694069 | 0.672695 | 7.49068981 | 0.74876288 |
| hsa-miR-3126-3p_st | 4.91701 | 0.491548 | 4.64334302 | 0.51987727 |
| hsa-miR-1296_st | 6.90035 | 0.511463 | 6.76773245 | 0.50642685 |
| hsa-miR-3127_st | 9.480921 | 0.5958 | 8.88771639 | 0.75023462 |
| hsa-miR-3128_st | 4.164292 | 0.358876 | 4.10721341 | 0.45186484 |
| hsa-miR-3129_st | 4.304161 | 0.38854 | 4.37728189 | 0.420934 |
| hsa-miR-3130-3p_st | 9.062106 | 0.595567 | 8.49020828 | 0.79300737 |
| hsa-miR-105-star_st | 4.101657 | 0.438842 | 3.93230779 | 0.41293377 |
| hsa-miR-3131_st | 12.176 | 0.445239 | 11.6946806 | 0.59437807 |
| hsa-miR-4255_st | 4.304628 | 0.441999 | 4.12888846 | 0.43303015 |
| hsa-miR-3133_st | 4.535888 | 1.183533 | 4.41228356 | 0.74970779 |
| hsa-miR-3134_st | 3.664814 | 0.507108 | 3.68434235 | 0.44503127 |
| hsa-miR-3135_st | 6.826625 | 0.611152 | 6.40583317 | 0.67177744 |
| hsa-miR-3136_st | 6.417549 | 0.64608 | 6.05961106 | 0.65733355 |
| hsa-miR-19b-1-star_st | 4.061083 | 0.407576 | 4.3058178 | 0.49761986 |
| hsa-miR-1247_st | 7.399755 | 0.56329 | 6.87797483 | 0.82094957 |
| hsa-miR-3139_st | 4.030071 | 0.397504 | 4.0847225 | 0.34945387 |
| hsa-miR-596_st | 6.630219 | 0.560824 | 6.34566826 | 0.68980371 |
| hsa-miR-3141_st | 14.63752 | 0.502169 | 14.1551188 | 0.54081713 |
| hsa-miR-3142_st | 3.228526 | 0.511483 | 3.33628987 | 0.54614208 |
| hsa-miR-1302_st | 3.784348 | 0.422743 | 3.61928841 | 0.41365303 |
| hsa-miR-3144-3p_st | 4.503057 | 0.551934 | 4.45822788 | 0.45194795 |
| hsa-miR-3144-5p_st | 5.473634 | 0.823243 | 5.36844046 | 0.75501648 |
| hsa-miR-3145_st | 3.481279 | 0.556547 | 3.37235746 | 0.58691575 |
| hsa-miR-3146_st | 4.806779 | 0.462714 | 4.59610965 | 0.65799197 |
| hsa-miR-346_st | 8.463695 | 0.537342 | 8.16209963 | 0.54362365 |
| hsa-miR-4256_st | 4.252585 | 0.428697 | 3.96820977 | 0.41703709 |
| hsa-miR-3149_st | 4.668127 | 0.508195 | 4.75573494 | 0.42668429 |
| hsa-miR-3150_st | 10.03205 | 0.583287 | 9.67824986 | 0.66288787 |
| hsa-miR-3151_st | 11.92253 | 0.487621 | 11.3666685 | 0.65451608 |
| hsa-miR-1470_st | 5.76857 | 0.484737 | 5.54979656 | 0.58428066 |
| hsa-miR-3153_st | 9.602147 | 0.618164 | 9.14607191 | 0.66505199 |
| hsa-miR-514_st | 3.335725 | 0.447224 | 3.44119198 | 0.51894863 |
| hsa-miR-3175_st | 12.96856 | 0.299339 | 12.818224 | 0.40814356 |
| hsa-miR-3156_st | 10.56189 | 0.4400761 | 0.5236603 | 0.37037246 |
| hsa-miR-504_st | 5.723855 | 0.588638 | 5.78217385 | 0.76664166 |
| hsa-miR-3158_st | 4.957519 | 0.728669 | 4.83228459 | 0.75523666 |
| hsa-miR-3159_st | 5.224462 | 0.510264 | 4.9953311 | 0.60751679 |
| hsa-miR-3160_st | 5.967081 | 0.650586 | 5.82750267 | 0.52071591 |
| hsa-miR-3161_st | 7.74174 | 0.992511 | 7.55017928 | 0.9167566 |
| hsa-miR-3162_st | 13.69697 | 0.350751 | 13.1835403 | 0.52519718 |
| hsa-miR-1234_st | 7.522424 | 0.471347 | 7.26479761 | 0.54590782 |
| hsa-miR-3164_st | 7.934438 | 0.693833 | 7.26708431 | 0.86761343 |
| hsa-miR-516b-star_st | 3.891291 | 0.443377 | 4.12304902 | 0.47847515 |
| hsa-miR-3166_st | 4.859359 | 0.424716 | 4.99582314 | 0.45108985 |
| hsa-miR-4327_st | 12.98085 | 0.429377 | 12.3343606 | 0.57077669 |
| hsa-miR-3168_st | 3.356534 | 0.493178 | 3.51162735 | 0.53724399 |
| hsa-miR-3169_st | 4.7004 | 0.457484 | 4.50867162 | 0.37410857 |
| hsa-miR-3170_st | 6.866305 | 0.750592 | 6.54416701 | 0.7710439 |
| hsa-miR-3171_st | 3.473133 | 0.446955 | 3.51801041 | 0.49328237 |
| hsa-miR-3172_st | 15.09805 | 0.755682 | 15.4141307 | 0.80483 |
| hsa-miR-374b-star_st | 3.741407 | 0.348141 | 4.06635283 | 0.5645087 |
| hsa-miR-34c-5p_st | 4.082741 | 0.371525 | 4.44666858 | 1.03597985 |
| hsa-miR-758_st | 3.658324 | 0.517302 | 3.69189652 | 0.76661976 |
| hsa-miR-3176_st | 11.91393 | 0.999656 | 11.8241466 | 0.74629134 |
| hsa-miR-606_st | 3.192972 | 0.506689 | 3.43406738 | 0.48633196 |
| hsa-miR-3178_st | 15.48585 | 0.510856 | 15.1609346 | 0.56100051 |
| hsa-miR-3179_st | 4.561842 | 0.868057 | 4.41737193 | 0.69982633 |
| hsa-miR-3180-3p_st | 14.78645 | 0.521448 | 14.3884118 | 0.60103458 |
| hsa-miR-597_st | 3.375527 | 0.531771 | 3.68733555 | 0.579442 |
| hsa-miR-148b-star_st | 3.353429 | 0.459222 | 3.67882454 | 0.65145919 |
| hsa-miR-3182_st | 4.134859 | 0.521338 | 4.18415679 | 0.55662767 |
| hsa-miR-3183_st | 6.107105 | 0.599916 | 5.72577147 | 0.55515728 |
| hsa-miR-144-star_st | 3.795629 | 0.537974 | 4.0415967 | 0.6441144 |
| hsa-miR-3185_st | 15.2779 | 0.555308 | 14.8536058 | 0.53780496 |
| hsa-miR-4270_st | 15.3228 | 0.454482 | 14.9270537 | 0.44340183 |
| hsa-miR-3186-5p_st | 5.55249 | 0.627668 | 5.3504552 | 0.61661896 |
| hsa-miR-3187_st | 13.49394 | 0.494512 | 12.8984145 | 0.59160091 |
| hsa-miR-3188_st | 14.00477 | 0.498515 | 13.354323 | 0.50134359 |
| hsa-miR-3189_st | 8.046312 | 0.717761 | 7.5100952 | 0.63406501 |
| hsa-miR-548b-3p_st | 3.75837 | 0.527389 | 3.92393399 | 0.60290205 |
| hsa-miR-99a-star_st | 4.904107 | 0.561379 | 5.28278655 | 0.91580408 |
| hsa-miR-376a-star_st | 2.947652 | 0.608765 | 3.09117543 | 0.65034026 |
| hsa-miR-216b_st | 3.847461 | 0.462002 | 4.18210728 | 0.53565292 |
| hsa-miR-3193_st | 6.892475 | 0.506815 | 6.77460892 | 0.54830453 |
| hsa-miR-548v_st | 3.260885 | 0.546773 | 3.4425624 | 0.55774687 |
| hsa-miR-26a-1-star_st | 3.511849 | 0.533379 | 3.39882581 | 0.41049936 |
| hsa-miR-1291_st | 9.050779 | 0.63812 | 8.86208373 | 0.8751353 |
| hsa-miR-3197_st | 15.20793 | 0.456222 | 14.7916453 | 0.613482 |
| hsa-miR-629_st | 7.743164 | 0.619386 | 8.2737559 | 0.63210909 |
| hsa-miR-3199_st | 5.593028 | 0.568101 | 5.51927359 | 0.65670565 |
| hsa-miR-190b_st | 3.090465 | 0.552555 | 3.24131796 | 0.51288518 |
| hsa-miR-32_st | 3.185348 | 0.593975 | 3.56481968 | 0.62000975 |
| hsa-miR-3200_st | 4.17715 | 0.468426 | 4.57396482 | 0.65615804 |
| hsa-miR-3201_st | 4.267973 | 0.504037 | 4.07083997 | 0.57408862 |
| hsa-miR-3202_st | 7.949926 | 0.86704 | 7.78409209 | 0.79332959 |
| hsa-miR-320a_st | 12.33395 | 0.43992 | 12.3652897 | 0.43638929 |
| hsa-miR-4289_st | 6.724997 | 0.693183 | 7.03055474 | 0.70200839 |
| hsa-miR-655_st | 3.052842 | 0.590458 | 3.32863594 | 0.60330143 |
| hsa-miR-320b_st | 11.34285 | 0.479626 | 11.6066704 | 0.66364498 |
| hsa-miR-219-5p_st | 3.070874 | 0.71297 | 3.3939776 | 0.6240275 |
| hsa-miR-323-3p_st | 4.370783 | 0.453501 | 4.47349771 | 0.73239938 |
| hsa-miR-323-5p_st | 11.14189 | 0.663054 | 10.4644945 | 0.71084037 |
| hsa-miR-323b-3p_st | 4.066809 | 0.639045 | 4.0036625 | 0.50797183 |
| hsa-miR-103-as_st | 3.886225 | 0.504493 | 4.33234024 | 0.48018738 |
| hsa-miR-424-star_st | 8.632444 | 0.433792 | 9.0418152 | 0.64384415 |
| hsa-miR-33a-star_st | 3.829405 | 0.623432 | 4.53181593 | 0.77903827 |
| hsa-miR-325_st | 4.967083 | 0.721525 | 4.67546839 | 0.83335797 |
| hsa-miR-590-3p_st | 3.2437 | 0.527488 | 3.29693449 | 0.71216016 |
| hsa-miR-328_st | 7.833323 | 0.740985 | 8.31790367 | 1.11451196 |
| hsa-miR-329_st | 3.894032 | 0.482935 | 4.36295644 | 1.02529234 |
| hsa-miR-32-star_st | 4.927833 | 0.478206 | 4.9257615 | 0.55632544 |
| hsa-miR-330-3p_st | 6.597377 | 0.51527 | 6.70560497 | 0.58792932 |
| hsa-miR-330-5p_st | 6.286831 | 0.610344 | 6.03818569 | 0.63197768 |
| hsa-miR-146a-star_st | 3.073914 | 0.521667 | 3.49961338 | 0.56226189 |
| hsa-miR-331-5p_st | 7.183827 | 0.577797 | 7.48843067 | 0.73426129 |
| hsa-miR-3167_st | 3.426018 | 0.427286 | 3.27558859 | 0.47420154 |
| hsa-miR-335-star_st | 3.958195 | 0.752706 | 3.9914167 | 0.75991322 |
| hsa-miR-337-3p_st | 4.499582 | 0.678846 | 4.29293712 | 0.55032248 |
| hsa-miR-337-5p_st | 3.9586 | 0.469901 | 4.04647209 | 0.56852148 |
| hsa-miR-421_st | 6.960999 | 0.658287 | 7.71710635 | 0.72901283 |
| hsa-miR-338-5p_st | 5.636557 | 0.432382 | 6.02253919 | 0.74880317 |
| hsa-miR-339-3p_st | 8.836054 | 0.518838 | 9.07904072 | 0.54197073 |
| hsa-miR-548a-5p_st | 4.496835 | 0.454132 | 4.74687915 | 0.39000992 |
| hsa-miR-33a_st | 3.07625 | 0.554997 | 3.1554349 | 0.67587877 |
| hsa-miR-590-5p_st | 3.523502 | 0.509156 | 4.02123453 | 0.71365623 |
| hsa-miR-33b_st | 3.39599 | 0.456871 | 3.56046677 | 0.54830819 |
| hsa-miR-616-star_st | 3.999118 | 0.524463 | 4.35974257 | 0.52361252 |
| hsa-miR-569_st | 3.841971 | 0.506813 | 3.88670845 | 0.49944099 |
| hsa-miR-340-star_st | 3.159949 | 0.506138 | 3.39729259 | 0.71232231 |
| hsa-miR-342-3p_st | 10.50075 | 1.195893 | 11.4170159 | 1.12296604 |
| hsa-miR-135b-star_st | 8.470475 | 0.891634 | 8.22988542 | 0.89613074 |
| hsa-miR-345_st | 10.30203 | 1.318505 | 10.8398276 | 1.41895486 |
| hsa-miR-1288_st | 5.280472 | 0.443864 | 4.97420276 | 0.43455924 |
| hsa-miR-135a_st | 3.123487 | 0.615175 | 3.45583379 | 0.84785539 |
| hsa-miR-34a-star_st | 5.505559 | 1.007936 | 6.91782236 | 1.34602229 |
| hsa-miR-34b_st | 5.204223 | 0.607549 | 5.27972121 | 0.68271962 |
| hsa-miR-4288_st | 4.475043 | 0.54204 | 5.01688664 | 0.5543019 |
| hsa-miR-222-star_st | 6.106742 | 0.909655 | 7.72599786 | 1.27140568 |
| hsa-miR-933_st | 11.2424 | 0.659944 | 11.7602279 | 0.62488114 |
| hsa-miR-361-3p_st | 6.651218 | 0.958945 | 7.54430313 | 0.95679198 |
| hsa-miR-224-star_st | 5.804651 | 0.474078 | 6.45540807 | 0.79859114 |
| hsa-miR-362-3p_st | 4.477435 | 0.865007 | 5.08892217 | 1.1270098 |
| hsa-miR-362-5p_st | 6.759382 | 0.720225 | 7.57210815 | 1.00828903 |
| hsa-miR-363_st | 5.718796 | 0.905945 | 6.01887139 | 1.36057189 |
| hsa-miR-363-star_st | 9.854413 | 0.618135 | 9.44281281 | 0.71875925 |
| hsa-miR-1979_st | 15.23059 | 0.659874 | 15.4654283 | 0.61680383 |
| hsa-miR-365-star_st | 12.33801 | 0.44289 | 11.6801618 | 0.56165947 |
| hsa-miR-548x_st | 3.005415 | 0.624034 | 3.19707894 | 0.67098945 |
| hsa-miR-367-star_st | 3.192781 | 0.582819 | 3.42140022 | 0.61720824 |
| hsa-miR-369-3p_st | 3.099533 | 0.513888 | 3.17872537 | 0.65428451 |
| hsa-miR-369-5p_st | 3.863914 | 0.349865 | 4.2476199 | 0.81772146 |
| hsa-miR-548l_st | 5.416045 | 0.490895 | 5.05825615 | 0.52841223 |
| hsa-miR-371-3p_st | 4.055385 | 0.441545 | 4.08350367 | 0.44632772 |
| hsa-miR-371-5p_st | 13.07245 | 0.515766 | 12.3968184 | 0.54398317 |
| hsa-miR-372_st | 4.449338 | 0.643097 | 4.23951688 | 0.39848701 |
| hsa-miR-373_st | 5.438272 | 0.601778 | 5.23734577 | 0.52330417 |
| hsa-miR-373-star_st | 12.34867 | 0.561983 | 11.6452636 | 0.65654465 |
| hsa-miR-374a_st | 4.636737 | 1.172011 | 5.56304278 | 1.31405758 |
| hsa-miR-374a-star_st | 3.574642 | 0.492143 | 3.88051884 | 0.51664318 |
| hsa-miR-500_st | 6.445738 | 0.645955 | 6.98979085 | 0.73691867 |
| hsa-miR-320c_st | 10.81818 | 0.65127 | 11.2028649 | 0.78211289 |
| hsa-miR-875-3p_st | 4.703813 | 0.40937 | 4.60366518 | 0.37552284 |
| hsa-miR-376a_st | 4.482481 | 0.692729 | 4.72564609 | 0.95050282 |
| hsa-miR-29b-1-star_st | 6.24022 | 0.726633 | 7.62149452 | 0.90431086 |
| hsa-miR-376b_st | 3.637573 | 0.484211 | 3.83434675 | 0.73888442 |
| hsa-miR-376c_st | 4.692923 | 0.919456 | 5.16942409 | 1.46401293 |
| hsa-miR-377_st | 3.596003 | 0.460698 | 3.64103045 | 0.84459352 |
| hsa-miR-377-star_st | 5.022027 | 0.667778 | 5.12315423 | 0.59711685 |
| hsa-miR-378_st | 9.023316 | 0.481853 | 9.21974786 | 1.13420711 |
| hsa-miR-378b_st | 9.152552 | 0.398945 | 8.79293666 | 0.50636488 |
| hsa-miR-378c_st | 9.324971 | 0.462952 | 9.32843634 | 0.85523297 |
| hsa-miR-378-star_st | 5.410873 | 0.447244 | 5.62438861 | 0.72935466 |
| hsa-miR-662_st | 7.130306 | 0.588838 | 7.89389425 | 0.79891862 |
| hsa-miR-379-star_st | 3.507107 | 0.539389 | 3.73461014 | 0.47555469 |
| hsa-miR-380_st | 3.462347 | 0.479189 | 3.56004782 | 0.57096373 |
| hsa-miR-380-star_st | 3.792815 | 0.44919 | 3.77925687 | 0.4834703 |
| hsa-miR-1274b_st | 14.24856 | 0.805557 | 15.0939516 | 0.64714015 |
| hsa-miR-382_st | 6.732189 | 0.526563 | 6.67731615 | 1.01707887 |
| hsa-miR-383_st | 6.337119 | 0.654392 | 6.0955376 | 0.6368297 |
| hsa-miR-384_st | 3.017593 | 0.605746 | 3.16667199 | 0.56585892 |
| hsa-miR-126-star_st | 3.489964 | 0.593257 | 3.57912631 | 0.59289848 |
| hsa-miR-409-5p_st | 4.571669 | 0.400622 | 4.6291478 | 0.55813454 |
| hsa-miR-410_st | 4.438634 | 0.323714 | 4.55839528 | 0.61591152 |
| hsa-miR-411_st | 4.585955 | 0.478241 | 4.62169062 | 0.58527949 |
| hsa-miR-411-star_st | 4.506773 | 0.498141 | 4.74029158 | 0.58905879 |
| hsa-miR-412_st | 5.14131 | 0.695059 | 4.91085477 | 0.40064634 |
| hsa-miR-3143_st | 3.251294 | 0.592874 | 3.2019943 | 0.68003472 |
| hsa-miR-422a_st | 9.865196 | 0.389244 | 9.86248215 | 0.77199038 |
| hsa-miR-628-5p_st | 3.795382 | 0.51616 | 4.23440721 | 0.6887173 |
| hsa-miR-423-5p_st | 13.59708 | 0.33684 | 13.1924806 | 0.42957103 |
| hsa-miR-27b-star_st | 6.210673 | 0.692693 | 7.55600605 | 0.72431805 |
| hsa-miR-499-5p_st | 3.86941 | 0.509375 | 4.46211531 | 0.82640057 |
| hsa-miR-425_st | 7.93508 | 1.124078 | 9.39941342 | 1.19158046 |
| hsa-miR-193b_st | 8.389325 | 1.217186 | 8.89681573 | 1.55820002 |
| hsa-miR-4252_st | 5.581837 | 0.434368 | 5.40953811 | 0.3989544 |
| hsa-miR-4253_st | 12.48343 | 0.492355 | 11.9432648 | 0.56793561 |
| hsa-miR-4254_st | 5.54837 | 0.59345 | 5.92594763 | 0.67038658 |
| hsa-miR-324-3p_st | 8.478484 | 0.583228 | 8.84526556 | 0.6639283 |
| hsa-miR-4329_st | 5.418088 | 0.524544 | 6.00249289 | 0.60048704 |
| hsa-miR-1251_st | 4.78554 | 0.609984 | 5.49829992 | 1.00945316 |
| hsa-miR-4258_st | 6.225926 | 0.699167 | 5.96051783 | 0.53546304 |
| hsa-miR-1274a_st | 10.55955 | 1.310195 | 11.9561369 | 0.93505051 |
| hsa-miR-425-star_st | 8.190254 | 0.450253 | 8.15469243 | 0.46760782 |
| hsa-miR-4260_st | 11.40847 | 0.475864 | 10.8592329 | 0.67178459 |
| hsa-miR-4261_st | 9.242025 | 0.521913 | 9.11020763 | 0.63120292 |
| hsa-miR-4262_st | 3.438188 | 0.536427 | 3.33774159 | 0.58025607 |
| hsa-miR-4263_st | 3.614392 | 0.385649 | 3.63672359 | 0.49692175 |
| hsa-miR-4264_st | 3.319984 | 0.462291 | 3.36627508 | 0.51985226 |
| hsa-miR-1280_st | 14.71578 | 0.840208 | 15.2467969 | 0.66100378 |
| hsa-miR-132-star_st | 4.280994 | 0.534235 | 5.47483704 | 1.39927309 |
| hsa-miR-4267_st | 7.061295 | 0.686072 | 6.7428723 | 0.54594867 |
| hsa-miR-4268_st | 3.783659 | 0.427158 | 3.7610505 | 0.62835083 |
| hsa-miR-133a_st | 5.982869 | 1.372575 | 6.24329978 | 2.19043142 |
| hsa-miR-888-star_st | 3.610145 | 0.548088 | 3.67991398 | 0.6132167 |
| hsa-miR-1260b_st | 14.82574 | 0.835098 | 15.392403 | 0.63811604 |
| hsa-miR-4272_st | 3.47699 | 0.489957 | 3.52486562 | 0.48349446 |
| hsa-miR-4273_st | 3.421672 | 0.495347 | 3.42999535 | 0.47380105 |
| hsa-miR-4274_st | 5.721386 | 0.399178 | 5.75269271 | 0.60431264 |
| hsa-miR-4275_st | 3.003178 | 0.564367 | 3.27753422 | 0.53135187 |
| hsa-miR-4276_st | 3.14034 | 0.633284 | 3.28994234 | 0.54521572 |
| hsa-miR-4277_st | 3.911188 | 0.427129 | 3.88289138 | 0.51752009 |
| hsa-miR-4278_st | 7.557067 | 0.661615 | 7.21779511 | 0.72547911 |
| hsa-miR-4279_st | 5.160887 | 0.642726 | 5.10971592 | 0.47616144 |
| hsa-miR-4280_st | 4.866567 | 0.651411 | 4.72243706 | 0.60563682 |
| hsa-miR-4281_st | 15.79123 | 0.598525 | 15.5460375 | 0.48164337 |
| hsa-miR-4282_st | 2.89293 | 0.451322 | 2.98124179 | 0.55963954 |
| hsa-miR-4283_st | 8.898519 | 0.522733 | 8.3747562 | 0.77248253 |
| hsa-miR-4284_st | 15.24454 | 0.740276 | 15.5341647 | 0.75142644 |
| hsa-miR-23b-star_st | 6.053939 | 0.595331 | 6.72867297 | 0.81386648 |
| hsa-miR-574-3p_st | 9.376738 | 0.847757 | 9.87041869 | 0.88650414 |
| hsa-miR-4287_st | 5.99126 | 0.632647 | 5.66087059 | 0.5206105 |
| hsa-miR-320e_st | 9.707901 | 0.699655 | 10.3660527 | 0.82396224 |
| hsa-miR-339-5p_st | 7.80776 | 1.124566 | 8.87590386 | 1.00393661 |
| hsa-miR-505_st | 5.843533 | 0.858985 | 6.66046067 | 1.09851375 |
| hsa-miR-4290_st | 5.99431 | 0.8299 | 5.91956479 | 0.73136108 |
| hsa-miR-200b-star_st | 7.077419 | 0.570595 | 7.54983797 | 1.11065203 |
| hsa-miR-4292_st | 8.704875 | 0.541092 | 8.14338347 | 0.73826757 |
| hsa-miR-4293_st | 5.216492 | 0.65563 | 5.02094834 | 0.53772545 |
| hsa-miR-1246_st | 13.26622 | 0.575666 | 13.7145373 | 0.68279781 |
| hsa-miR-4295_st | 3.580301 | 0.432054 | 3.66800483 | 0.511011 |
| hsa-miR-4296_st | 7.418634 | 0.672207 | 6.99650716 | 0.62783237 |
| hsa-miR-4297_st | 5.616406 | 0.950787 | 5.39385531 | 0.75015773 |
| hsa-miR-4298_st | 13.21964 | 0.314554 | 12.7102554 | 0.54889567 |
| hsa-miR-218_st | 5.411662 | 1.070348 | 6.9434086 | 1.54272718 |
| hsa-miR-4286_st | 13.90234 | 1.156584 | 14.8889175 | 0.71083579 |
| hsa-miR-4301_st | 3.274693 | 0.608205 | 3.5209961 | 0.73725687 |
| hsa-miR-4302_st | 6.351504 | 0.684166 | 5.95340305 | 0.52403012 |
| hsa-miR-365_st | 4.901677 | 1.020452 | 5.22420174 | 1.00715641 |
| hsa-miR-4304_st | 6.922901 | 0.821131 | 6.59789057 | 0.76151483 |
| hsa-miR-4305_st | 3.958309 | 0.572152 | 4.09029581 | 0.5724331 |
| hsa-miR-4306_st | 9.382493 | 0.54642 | 9.56618029 | 0.71024414 |
| hsa-miR-26a_st | 14.44511 | 0.849545 | 15.1766251 | 0.73408249 |
| hsa-miR-4308_st | 3.54228 | 0.541698 | 3.66878775 | 0.52848429 |
| hsa-miR-4309_st | 4.382955 | 0.371151 | 4.50123913 | 0.41470692 |
| hsa-miR-186_st | 6.114806 | 0.94683 | 7.09673473 | 1.34625136 |
| hsa-miR-4310_st | 5.647787 | 0.836996 | 5.32953346 | 0.86168613 |
| hsa-miR-4311_st | 5.716136 | 0.600937 | 5.61624172 | 0.55189471 |
| hsa-miR-4312_st | 6.444188 | 0.661073 | 6.12500058 | 0.80599854 |
| hsa-miR-4313_st | 5.997393 | 0.598278 | 6.0552011 | 0.48793013 |
| hsa-miR-4314_st | 9.528721 | 0.725752 | 8.95564551 | 1.13974391 |
| hsa-miR-4315_st | 4.53505 | 0.686156 | 4.48984093 | 0.57823059 |
| hsa-miR-149_st | 6.414102 | 0.663122 | 7.4058717 | 1.17292168 |
| hsa-miR-106b-star_st | 7.490213 | 0.653656 | 8.24710094 | 0.80436565 |
| hsa-miR-4318_st | 4.900414 | 0.448643 | 4.77462316 | 0.40635958 |
| hsa-miR-4319_st | 4.848578 | 0.733648 | 4.8486114 | 0.62689495 |
| hsa-miR-29b-2-star_st | 7.401457 | 0.660914 | 8.37233542 | 0.69725353 |
| hsa-miR-432_st | 7.842229 | 0.574901 | 7.41811807 | 0.84475303 |
| hsa-miR-4320_st | 4.102699 | 0.423987 | 4.24454038 | 0.44298839 |
| hsa-miR-522_st | 3.995208 | 0.523267 | 4.03370672 | 0.59723027 |
| hsa-miR-4322_st | 13.44026 | 0.525556 | 12.8168962 | 0.50658407 |
| hsa-miR-4323_st | 5.305741 | 0.579646 | 5.14149768 | 0.49121558 |
| hsa-miR-93-star_st | 5.092212 | 0.647852 | 5.86163896 | 0.66531905 |
| hsa-miR-4325_st | 3.44381 | 0.553924 | 3.67119081 | 0.57265843 |
| hsa-miR-4326_st | 4.995116 | 0.418015 | 5.00342016 | 0.44784406 |
| hsa-miR-874_st | 13.12208 | 0.500459 | 12.6181094 | 0.52161199 |
| hsa-miR-4328_st | 3.406979 | 0.501841 | 3.52920794 | 0.46670436 |
| hsa-let-7e-star_st | 4.577797 | 0.56767 | 5.4166649 | 0.80311751 |
| hsa-miR-720_st | 15.68662 | 0.689857 | 15.8460648 | 0.68895065 |
| hsa-miR-433_st | 5.951695 | 0.602062 | 5.65434308 | 0.61989667 |
| hsa-miR-4330_st | 5.537312 | 1.047828 | 5.43901994 | 1.02422832 |
| hsa-miR-448_st | 3.983789 | 0.636383 | 3.89808326 | 0.4595336 |
| hsa-miR-449a_st | 3.94546 | 0.345635 | 4.31204086 | 0.6831466 |
| hsa-miR-449b_st | 5.331669 | 0.519455 | 5.35550478 | 0.47661579 |
| hsa-miR-1975_st | 15.72165 | 0.603304 | 15.7873879 | 0.53102109 |
| hsa-miR-449c_st | 7.707047 | 1.004191 | 7.0925273 | 0.81846888 |
| hsa-miR-449c-star_st | 4.619954 | 0.391777 | 4.49859221 | 0.61588559 |
| hsa-miR-1_st | 5.048139 | 1.203957 | 5.62481631 | 2.18671333 |
| hsa-miR-450b-3p_st | 2.963581 | 0.597882 | 3.02656302 | 0.59769935 |
| hsa-miR-450b-5p_st | 3.024492 | 0.642531 | 3.15571341 | 0.69457909 |
| hsa-miR-451_st | 13.09447 | 1.088968 | 13.1823818 | 1.36373878 |
| hsa-miR-452_st | 6.89586 | 0.680286 | 7.04214188 | 0.98586868 |
| hsa-miR-452-star_st | 3.766212 | 0.348038 | 3.75236573 | 0.45794567 |
| hsa-miR-548b-5p_st | 4.28699 | 0.448481 | 4.06331629 | 0.47432124 |
| hsa-miR-454-star_st | 3.846234 | 0.519613 | 3.99982615 | 0.65287472 |
| hsa-miR-455-3p_st | 8.120562 | 0.982113 | 8.54959167 | 1.19648838 |
| hsa-miR-455-5p_st | 4.374861 | 0.763842 | 4.70894293 | 0.94706117 |
| hsa-miR-466_st | 5.581714 | 0.822872 | 5.81929646 | 0.64127151 |
| hsa-miR-483-3p_st | 6.509845 | 0.64951 | 6.50626594 | 0.7131139 |
| hsa-miR-17_st | 9.79197 | 1.024657 | 11.0722725 | 1.22014996 |
| hsa-miR-320d_st | 9.937922 | 0.827667 | 10.5477279 | 0.92712929 |
| hsa-miR-485-3p_st | 5.775795 | 0.661254 | 5.82710954 | 0.84327991 |
| hsa-miR-485-5p_st | 7.378994 | 0.660122 | 7.00475322 | 0.67126201 |
| hsa-miR-486-3p_st | 10.82569 | 0.705103 | 10.3459594 | 0.81830238 |
| hsa-miR-486-5p_st | 8.702254 | 1.160757 | 8.18569382 | 1.4008656 |
| hsa-miR-644_st | 3.603317 | 0.488474 | 3.58195274 | 0.48914084 |
| hsa-miR-3152_st | 7.816206 | 0.855584 | 7.17050225 | 0.83648413 |
| hsa-miR-488_st | 3.655323 | 0.470916 | 3.82583644 | 0.52037269 |
| hsa-miR-488-star_st | 3.76761 | 0.552798 | 3.7876456 | 0.48068826 |
| hsa-miR-450a_st | 4.239673 | 0.612996 | 5.41678463 | 1.25037701 |
| hsa-miR-20a_st | 9.264828 | 1.25689 | 10.7857162 | 1.2087414 |
| hsa-miR-484_st | 7.89797 | 0.865901 | 8.67748389 | 0.91345554 |
| hsa-miR-491-3p_st | 4.795182 | 0.455728 | 5.0025796 | 0.78658515 |
| hsa-miR-491-5p_st | 9.393372 | 0.476622 | 9.35477015 | 0.53315181 |
| hsa-miR-197_st | 7.507565 | 0.861805 | 8.17656423 | 1.21778923 |
| hsa-miR-493_st | 5.773862 | 0.56158 | 5.83422506 | 0.55501001 |
| hsa-miR-493-star_st | 4.373008 | 0.532062 | 4.76621919 | 1.02994215 |
| hsa-miR-494_st | 15.32919 | 0.634427 | 14.8116457 | 0.86601387 |
| hsa-miR-495_st | 3.839973 | 0.642191 | 4.42929326 | 1.17138718 |
| hsa-miR-496_st | 3.281923 | 0.423251 | 3.42757916 | 0.50803811 |
| hsa-miR-497_st | 8.783128 | 1.276071 | 9.03420629 | 1.24078123 |
| hsa-miR-497-star_st | 3.590765 | 0.384453 | 3.68907471 | 0.52598146 |
| hsa-miR-498_st | 12.36457 | 0.479427 | 11.7317108 | 0.60922714 |
| hsa-miR-499-3p_st | 3.959258 | 0.365279 | 4.14673596 | 0.41970242 |
| hsa-miR-24-1-star_st | 4.241738 | 0.911295 | 5.3154219 | 1.01210908 |
| hsa-miR-340_st | 5.039813 | 0.975807 | 6.3171148 | 1.24834241 |
| hsa-miR-500b_st | 4.883788 | 0.505497 | 5.17853319 | 0.60974842 |
| hsa-miR-500-star_st | 9.454917 | 0.404851 | 9.27881846 | 0.53102515 |
| hsa-miR-501-3p_st | 9.489702 | 0.396711 | 9.19382791 | 0.41988179 |
| hsa-miR-501-5p_st | 6.366745 | 0.507204 | 6.39551152 | 0.58207992 |
| hsa-miR-502-3p_st | 9.215907 | 0.37784 | 9.03401862 | 0.70624163 |
| hsa-miR-502-5p_st | 5.38639 | 0.474146 | 5.4049096 | 0.43908919 |
| hsa-miR-503_st | 10.85252 | 0.687416 | 10.4574649 | 0.57696176 |
| hsa-miR-192_st | 7.16421 | 1.283466 | 8.57242898 | 1.2569885 |
| hsa-let-7g_st | 13.26945 | 0.940059 | 14.2205466 | 0.76825716 |
| hsa-miR-505-star_st | 7.603197 | 0.485573 | 7.48544292 | 0.61411802 |
| hsa-miR-506_st | 3.840776 | 0.456902 | 3.9209644 | 0.54489314 |
| hsa-miR-507_st | 3.49053 | 0.538168 | 3.59760566 | 0.54359176 |
| hsa-miR-508-3p_st | 3.322743 | 0.573838 | 3.49436893 | 0.61462929 |
| hsa-miR-508-5p_st | 9.329902 | 0.586465 | 8.92019429 | 0.64462028 |
| hsa-miR-509-3-5p_st | 7.170518 | 0.954568 | 6.94556958 | 0.86770187 |
| hsa-miR-509-3p_st | 6.0357 | 0.569391 | 5.83742865 | 0.67735157 |
| hsa-miR-106a_st | 9.580399 | 1.050156 | 10.9678413 | 1.20305162 |
| hsa-miR-106b_st | 8.560082 | 1.145483 | 10.0632788 | 1.14719127 |
| hsa-miR-511_st | 3.28459 | 0.5506 | 3.22672588 | 0.4977934 |
| hsa-miR-512-3p_st | 5.404369 | 0.60803 | 5.1041096 | 0.41144425 |
| hsa-miR-512-5p_st | 5.902081 | 0.64687 | 5.712204 | 0.52211898 |
| hsa-miR-513a-3p_st | 2.994974 | 0.566369 | 3.14280657 | 0.63463867 |
| hsa-miR-23a_st | 13.84016 | 0.944088 | 14.7248576 | 0.93867496 |
| hsa-miR-25_st | 10.16535 | 1.016639 | 11.3504046 | 0.93370536 |
| hsa-miR-598_st | 5.949416 | 0.912461 | 7.17584265 | 1.10670963 |
| hsa-miR-1260_st | 11.09415 | 0.984005 | 12.1430687 | 0.85562814 |
| hsa-miR-514b-3p_st | 3.62973 | 0.485733 | 3.66120933 | 0.59376863 |
| hsa-miR-514b-5p_st | 11.22534 | 0.481423 | 10.792225 | 0.63788446 |
| hsa-miR-515-3p_st | 4.337475 | 0.492399 | 4.21287078 | 0.52694377 |
| hsa-miR-515-5p_st | 3.636275 | 0.493366 | 3.57128743 | 0.47186337 |
| hsa-miR-516a-3p_st | 3.874549 | 0.482899 | 3.94242832 | 0.47584692 |
| hsa-miR-27a_st | 11.90714 | 1.077963 | 13.250889 | 1.05383889 |
| hsa-miR-100_st | 12.23844 | 1.076291 | 12.9411831 | 1.08306592 |
| hsa-miR-101_st | 5.712467 | 1.184111 | 7.18380992 | 1.42488449 |
| hsa-miR-517a_st | 3.223932 | 0.600189 | 3.4188867 | 0.53290134 |
| hsa-miR-517b_st | 3.750063 | 0.377614 | 3.90609688 | 0.4930514 |
| hsa-miR-517c_st | 2.829136 | 0.557962 | 2.87674096 | 0.50174656 |
| hsa-miR-517-star_st | 5.420155 | 0.565331 | 5.31236377 | 0.45935484 |
| hsa-miR-518a-3p_st | 3.763268 | 0.505304 | 3.66876317 | 0.40393229 |
| hsa-miR-518a-5p_st | 7.475702 | 0.785792 | 7.06197331 | 0.87873414 |
| hsa-miR-518b_st | 4.12342 | 0.561781 | 4.21222673 | 0.52155971 |
| hsa-miR-518c_st | 3.099383 | 0.515476 | 3.1984902 | 0.5989285 |
| hsa-miR-301a_st | 4.246788 | 0.835949 | 5.47677515 | 1.05413779 |
| hsa-miR-518d-3p_st | 4.241136 | 0.339449 | 4.17109377 | 0.48445969 |
| hsa-miR-518d-5p_st | 6.815643 | 0.645825 | 6.70277484 | 0.71478134 |
| hsa-miR-518e_st | 3.042633 | 0.482675 | 3.33469849 | 0.5861646 |
| hsa-let-7f_st | 14.23317 | 0.925492 | 15.0157695 | 0.69288509 |
| hsa-miR-518f_st | 3.243074 | 0.566673 | 3.34818081 | 0.64342434 |
| hsa-miR-1826_st | 15.84369 | 0.630797 | 15.8044054 | 0.64118187 |
| hsa-miR-519a_st | 3.102453 | 0.617387 | 3.05705037 | 0.56978104 |
| hsa-miR-519a-star_st | 6.292434 | 0.800573 | 5.91004117 | 0.69477031 |
| hsa-miR-519b-3p_st | 3.391533 | 0.673187 | 3.48370995 | 0.57490958 |
| hsa-miR-1283_st | 3.974093 | 0.36236 | 3.82132284 | 0.48529743 |
| hsa-miR-519c-3p_st | 2.763353 | 0.597868 | 2.77564533 | 0.67459092 |
| hsa-miR-519c-5p_st | 6.619341 | 0.750737 | 6.32971949 | 0.86214798 |
| hsa-miR-519d_st | 3.780949 | 0.414049 | 3.90846648 | 0.54149127 |
| hsa-miR-519e_st | 3.859283 | 0.464531 | 4.2863061 | 0.56499953 |
| hsa-miR-519e-star_st | 7.364359 | 0.893006 | 6.97192634 | 0.64670384 |
| hsa-miR-520a-3p_st | 3.713982 | 0.48883 | 3.78797954 | 0.55124523 |
| hsa-miR-1908_st | 15.86571 | 0.66346 | 15.8169227 | 0.64455528 |
| hsa-miR-520b_st | 3.395918 | 0.477902 | 3.49234122 | 0.41356443 |
| hsa-miR-520c-3p_st | 4.1761 | 0.457159 | 4.28010158 | 0.52891463 |
| hsa-miR-520c-5p_st | 6.898663 | 0.726649 | 6.85487436 | 0.63179145 |
| hsa-miR-181a-star_st | 5.624778 | 1.056565 | 6.85626107 | 1.02657788 |
| hsa-miR-21_st | 11.22082 | 1.568694 | 13.6241237 | 1.457782 |
| hsa-miR-520e_st | 3.399161 | 0.552609 | 3.37877863 | 0.58115922 |
| hsa-miR-520f_st | 3.615821 | 0.570402 | 3.88710761 | 0.41264357 |
| hsa-miR-520g_st | 3.65981 | 0.469938 | 3.72944274 | 0.59827373 |
| hsa-miR-520h_st | 3.790587 | 0.515831 | 3.85926728 | 0.47191293 |
| hsa-miR-521_st | 3.605736 | 0.424582 | 3.93641888 | 0.57997103 |
| hsa-miR-302a-star_st | 4.062561 | 0.51412 | 3.92646632 | 0.40902817 |
| hsa-miR-886-3p_st | 11.01403 | 1.293679 | 11.9191302 | 1.18380636 |
| hsa-miR-523_st | 3.515156 | 0.48083 | 3.54230355 | 0.57369949 |
| hsa-miR-29a_st | 12.96012 | 1.134281 | 14.3356242 | 0.99042952 |
| hsa-miR-524-3p_st | 4.331525 | 0.486411 | 4.13337971 | 0.46221807 |
| hsa-miR-142-3p_st | 3.190812 | 0.672238 | 3.179795 | 0.78721002 |
| hsa-miR-525-3p_st | 3.667337 | 0.501324 | 3.45615634 | 0.53142411 |
| hsa-miR-525-5p_st | 6.49846 | 0.63598 | 6.39949527 | 0.62196612 |
| hsa-miR-762_st | 15.86588 | 0.663213 | 15.783934 | 0.60212851 |
| hsa-miR-423-3p_st | 9.258395 | 0.708732 | 10.2291543 | 0.78826496 |
| hsa-miR-449b-star_st | 7.926721 | 0.708969 | 7.58301053 | 0.69732249 |
| hsa-miR-527_st | 7.712253 | 0.75801 | 7.30018728 | 0.7494596 |
| hsa-miR-532-3p_st | 7.89441 | 0.904689 | 8.33079 | 0.86947854 |
| hsa-miR-532-5p_st | 7.947601 | 0.883256 | 8.5081953 | 0.82768072 |
| hsa-miR-539_st | 4.500196 | 0.588345 | 4.69314062 | 0.59597677 |
| hsa-let-7a_st | 14.31841 | 0.914189 | 15.0463656 | 0.61498521 |
| hsa-miR-541-star_st | 6.573895 | 0.478082 | 6.34530855 | 0.49970764 |
| hsa-miR-542-3p_st | 6.804265 | 0.992739 | 7.07715023 | 1.2745053 |
| hsa-miR-542-5p_st | 7.681975 | 0.538284 | 8.36918251 | 0.65137291 |
| hsa-miR-543_st | 4.994683 | 0.443972 | 5.03755994 | 0.83926679 |
| hsa-miR-544_st | 3.084294 | 0.655135 | 3.12600645 | 0.63498258 |
| hsa-miR-544b_st | 4.125303 | 0.484133 | 3.98662235 | 0.43445393 |
| hsa-miR-545_st | 3.238889 | 0.582825 | 3.25339113 | 0.60545204 |
| hsa-miR-545-star_st | 2.672494 | 0.644092 | 2.94542447 | 0.65516109 |
| hsa-miR-548a-3p_st | 2.978628 | 0.635474 | 3.06031912 | 0.68061935 |
| hsa-miR-2861_st | 15.87275 | 0.672343 | 15.7986233 | 0.61139366 |
| hsa-miR-31_st | 8.440254 | 1.863791 | 11.1964669 | 3.10840587 |
| hsa-miR-367_st | 3.292134 | 0.661369 | 3.26677948 | 0.60284233 |
| hsa-miR-548c-3p_st | 2.737987 | 0.80695 | 3.04263323 | 0.76570793 |
| hsa-miR-548c-5p_st | 4.904636 | 0.881415 | 5.11002594 | 0.69642675 |
| hsa-miR-548d-3p_st | 3.580087 | 0.531378 | 3.69483712 | 0.57932925 |
| hsa-miR-548d-5p_st | 4.523206 | 0.630285 | 4.62630546 | 0.60136075 |
| hsa-miR-548e_st | 3.10315 | 0.578037 | 3.1343633 | 0.59012984 |
| hsa-miR-548f_st | 3.3415 | 0.594306 | 3.3902609 | 0.54773622 |
| hsa-miR-548g_st | 2.991687 | 0.741289 | 3.13434961 | 0.72528329 |
| hsa-miR-548h_st | 3.667037 | 0.535998 | 4.03062696 | 0.48667257 |
| hsa-miR-548i_st | 4.372465 | 0.483515 | 4.41301748 | 0.50912062 |
| hsa-miR-548j_st | 4.708382 | 0.525985 | 4.59511735 | 0.51477175 |
| hsa-miR-548k_st | 3.72579 | 0.438241 | 3.78470023 | 0.44518082 |
| hsa-miR-526b-star_st | 3.357738 | 0.608487 | 3.33495448 | 0.61714466 |
| hsa-miR-548m_st | 3.950536 | 0.429382 | 3.88094571 | 0.53354887 |
| hsa-miR-548n_st | 3.377699 | 0.480864 | 3.5718754 | 0.54451641 |
| hsa-miR-548o_st | 2.924489 | 0.583211 | 3.03013544 | 0.55306492 |
| hsa-miR-548p_st | 3.136836 | 0.519217 | 3.20336691 | 0.59583593 |
| hsa-miR-548q_st | 9.800167 | 0.711229 | 9.0359398 | 0.86935363 |
| hsa-miR-548s_st | 3.36516 | 0.460472 | 3.3430032 | 0.49911645 |
| hsa-miR-548t_st | 4.135269 | 0.48972 | 4.08933053 | 0.54602671 |
| hsa-miR-548u_st | 3.099664 | 0.670893 | 3.43524819 | 0.76055943 |
| hsa-miR-638_st | 15.87497 | 0.674232 | 15.8642733 | 0.69383676 |
| hsa-miR-548w_st | 4.628354 | 0.672221 | 4.69124433 | 0.65516668 |
| hsa-miR-3119_st | 3.131661 | 0.599412 | 3.01071918 | 0.53389413 |
| hsa-miR-549_st | 4.087185 | 0.398277 | 4.06740003 | 0.47263172 |
| hsa-miR-205_st | 6.382853 | 1.872936 | 7.30235676 | 3.24162491 |
| hsa-miR-550-star_st | 4.781098 | 0.482277 | 5.2048318 | 0.78400216 |
| hsa-miR-551a_st | 4.642474 | 0.352147 | 4.61607942 | 0.60103223 |
| hsa-miR-93_st | 9.274462 | 0.930166 | 10.5909162 | 0.89380237 |
| hsa-miR-551b-star_st | 11.54558 | 0.463516 | 11.5685504 | 0.49574194 |
| hsa-let-7d-star_st | 8.01121 | 0.888434 | 8.73627063 | 1.44440283 |
| hsa-miR-553_st | 3.477332 | 0.519658 | 3.70598808 | 0.4875743 |
| hsa-miR-554_st | 4.233594 | 0.475235 | 4.15368728 | 0.43677016 |
| hsa-miR-555_st | 4.196198 | 0.420081 | 4.01222228 | 0.42637863 |
| hsa-miR-556-3p_st | 3.62307 | 0.607436 | 3.457876 | 0.54916026 |
| hsa-miR-556-5p_st | 3.131472 | 0.670532 | 3.26558836 | 0.5140776 |
| hsa-miR-557_st | 12.38797 | 0.500908 | 11.8334731 | 0.63095953 |
| hsa-miR-558_st | 3.364199 | 0.512671 | 3.56553553 | 0.48236096 |
| hsa-miR-559_st | 3.448604 | 0.543165 | 3.52031487 | 0.55445041 |
| hsa-miR-561_st | 3.390146 | 0.575099 | 3.43733463 | 0.54899155 |
| hsa-miR-562_st | 3.360453 | 0.588345 | 3.41536786 | 0.52407007 |
| hsa-miR-563_st | 4.188824 | 0.486736 | 4.19879075 | 0.46450831 |
| hsa-miR-361-5p_st | 10.74843 | 0.808392 | 11.8573382 | 0.82064345 |
| hsa-miR-566_st | 6.748361 | 0.548755 | 6.45831976 | 0.45860744 |
| hsa-miR-567_st | 4.205559 | 0.477944 | 3.99911103 | 0.60398126 |
| hsa-miR-568_st | 3.252494 | 0.600258 | 3.25321751 | 0.61304382 |
| hsa-miR-29c_st | 11.25493 | 1.106231 | 12.5832332 | 1.09863175 |
| hsa-miR-570_st | 3.35764 | 0.532831 | 3.57219239 | 0.49039663 |
| hsa-miR-571_st | 4.460428 | 0.447366 | 4.39919835 | 0.47158971 |
| hsa-miR-572_st | 14.65858 | 0.554941 | 14.1112417 | 0.57770232 |
| hsa-miR-573_st | 3.450177 | 0.50741 | 3.48885443 | 0.49134066 |
| hsa-miR-132_st | 6.380453 | 0.900662 | 8.01278178 | 1.25535559 |
| hsa-miR-574-5p_st | 8.055551 | 0.390453 | 8.00776417 | 0.41509893 |
| hsa-let-7d_st | 13.8237 | 0.937665 | 14.6395953 | 0.67890386 |
| hsa-miR-576-3p_st | 4.835093 | 0.639694 | 4.6914083 | 0.5788278 |
| hsa-miR-576-5p_st | 4.09447 | 0.55129 | 4.03896274 | 0.60077985 |
| hsa-miR-577_st | 3.433406 | 0.58579 | 3.39783603 | 0.54297977 |
| hsa-miR-578_st | 4.279217 | 0.503452 | 4.74841374 | 0.49801171 |
| hsa-miR-579_st | 3.512209 | 0.465282 | 3.63837976 | 0.62254428 |
| hsa-miR-580_st | 3.389652 | 0.535233 | 3.36288777 | 0.56397498 |
| hsa-miR-191_st | 11.95713 | 1.019289 | 13.1276483 | 0.91076401 |
| hsa-miR-582-3p_st | 4.134722 | 0.551934 | 4.07203176 | 0.4802804 |
| hsa-miR-582-5p_st | 3.651056 | 0.810482 | 3.56044359 | 0.71112644 |
| hsa-miR-1915_st | 15.87686 | 0.67742 | 15.854516 | 0.67119738 |
| hsa-let-7c_st | 13.82209 | 1.007105 | 14.6258993 | 0.68780309 |
| hsa-miR-585_st | 3.888395 | 0.52089 | 3.98546492 | 0.60852002 |
| hsa-miR-586_st | 3.259731 | 0.585465 | 3.29787831 | 0.54890185 |
| hsa-miR-324-5p_st | 8.345719 | 0.998362 | 9.55904048 | 0.98291148 |
| hsa-miR-588_st | 5.356442 | 0.510705 | 5.25973662 | 0.46223529 |
| hsa-miR-589_st | 4.826808 | 0.532217 | 5.03222025 | 0.56481849 |
| hsa-miR-589-star_st | 5.175403 | 0.552334 | 5.02221756 | 0.461047 |
| hsa-miR-188-5p_st | 8.701981 | 0.570579 | 8.01556844 | 0.64230462 |
| hsa-miR-103_st | 12.05687 | 0.974842 | 13.2364783 | 0.83619116 |
| hsa-miR-591_st | 4.180877 | 0.502979 | 4.06804151 | 0.51249386 |
| hsa-miR-592_st | 3.349657 | 0.451327 | 3.6368761 | 0.66362634 |
| hsa-miR-593_st | 6.228812 | 0.589272 | 5.93167992 | 0.51808009 |
| hsa-miR-23b_st | 13.85558 | 0.915987 | 14.8615518 | 0.83668228 |
| hsa-miR-595_st | 7.342816 | 0.49474 | 7.03989249 | 0.5281987 |
| hsa-miR-99a_st | 12.11869 | 1.085738 | 13.0004023 | 1.27045265 |
| hsa-miR-15b_st | 9.960097 | 0.966655 | 11.0795985 | 1.29203982 |
| hsa-miR-135b_st | 3.82595 | 1.003119 | 4.96086975 | 1.44223142 |
| hsa-miR-599_st | 3.08883 | 0.563441 | 3.20512891 | 0.5582047 |
| hsa-miR-600_st | 6.937915 | 0.857592 | 6.88338917 | 0.89473142 |
| hsa-miR-107_st | 11.86548 | 1.033942 | 13.0934488 | 0.90151188 |
| hsa-miR-602_st | 14.23869 | 0.512647 | 13.7394546 | 0.54026734 |
| hsa-miR-603_st | 2.975714 | 0.478973 | 3.10984305 | 0.52585439 |
| hsa-miR-3065-5p_st | 4.189776 | 0.77206 | 5.53365472 | 1.385922 |
| hsa-miR-605_st | 5.608277 | 0.846822 | 5.58333849 | 0.67857659 |
| hsa-miR-24_st | 12.62531 | 0.957253 | 13.9250901 | 0.90967618 |
| hsa-miR-607_st | 3.465717 | 0.530031 | 3.67987038 | 0.60175903 |
| hsa-miR-608_st | 9.715185 | 0.615282 | 9.23127657 | 0.66311245 |
| hsa-miR-609_st | 3.719508 | 0.494947 | 4.03967366 | 0.47646311 |
| hsa-miR-3196_st | 15.87736 | 0.67809 | 15.8824716 | 0.71028462 |
| hsa-miR-26b_st | 11.83907 | 1.259424 | 13.1464841 | 1.20134771 |
| hsa-miR-28-5p_st | 8.94783 | 1.023351 | 10.1279269 | 1.1313323 |
| hsa-miR-613_st | 4.240156 | 0.550972 | 4.18330013 | 0.34438442 |
| hsa-miR-338-3p_st | 4.367939 | 0.827962 | 5.66948339 | 1.57862627 |
| hsa-miR-615-3p_st | 7.476724 | 0.670789 | 7.2177705 | 0.63536172 |
| hsa-miR-454_st | 5.38686 | 1.208853 | 7.03521906 | 1.41553134 |
| hsa-miR-616_st | 7.934369 | 0.601901 | 7.87628515 | 0.67416341 |
| hsa-miR-151-5p_st | 11.44928 | 1.008441 | 12.6499958 | 0.8956519 |
| hsa-miR-617_st | 7.287681 | 1.02451 | 6.84066861 | 0.83430121 |
| hsa-miR-128_st | 7.732543 | 1.189345 | 9.04873541 | 1.05143975 |
| hsa-miR-619_st | 4.681172 | 0.34848 | 4.58496141 | 0.31563468 |
| hsa-miR-620_st | 3.891968 | 0.486872 | 3.71820456 | 0.53975462 |
| hsa-miR-621_st | 3.964123 | 0.440343 | 4.20913005 | 0.49817777 |
| hsa-let-7b_st | 12.88841 | 1.132557 | 13.7900124 | 0.90138505 |
| hsa-miR-623_st | 10.15922 | 0.570529 | 9.59605312 | 0.58480754 |
| hsa-miR-624_st | 4.169795 | 0.448953 | 4.3762814 | 0.55822927 |
| hsa-miR-624-star_st | 4.283074 | 0.444894 | 4.37592611 | 0.55369432 |
| hsa-miR-183_st | 5.896297 | 1.300923 | 7.04951101 | 1.51315999 |
| hsa-miR-30b_st | 12.19333 | 1.11744 | 13.5169929 | 1.07723288 |
| hsa-miR-626_st | 3.486285 | 0.412762 | 3.57027648 | 0.50105984 |
| hsa-miR-627_st | 3.391929 | 0.476413 | 3.50703062 | 0.54436672 |
| hsa-miR-628-3p_st | 11.27074 | 0.681103 | 10.6728633 | 1.15733268 |
| hsa-miR-31-star_st | 4.231425 | 1.424487 | 7.18659167 | 3.13543855 |
| hsa-miR-218-1-star_st | 3.87161 | 0.593967 | 3.89083394 | 0.60424333 |
| hsa-miR-629-star_st | 5.548995 | 0.7401 | 5.97713246 | 0.55937671 |
| hsa-miR-630_st | 8.696618 | 0.854998 | 7.90287743 | 1.06960098 |
| hsa-miR-631_st | 7.499833 | 0.725022 | 6.80270554 | 0.84571171 |
| hsa-miR-632_st | 4.641951 | 0.628312 | 4.74444294 | 0.53425603 |
| hsa-miR-633_st | 3.319096 | 0.433817 | 3.43047285 | 0.4822794 |
| hsa-miR-634_st | 5.909138 | 1.006653 | 5.96391768 | 0.9616405 |
| hsa-miR-635_st | 5.380339 | 0.607 | 5.25686584 | 0.609278 |
| hsa-miR-203_st | 7.026829 | 1.674033 | 8.40698177 | 1.938087 |
| hsa-miR-331-3p_st | 9.604678 | 1.207767 | 10.4486235 | 0.93750986 |
| hsa-miR-125b_st | 14.16815 | 0.956978 | 15.0986673 | 0.75828826 |
| hsa-let-7i_st | 13.08639 | 1.115109 | 14.3904181 | 0.98969489 |
| hsa-miR-640_st | 5.981111 | 0.645478 | 5.83314124 | 0.42916113 |
| hsa-miR-641_st | 4.863604 | 0.67368 | 4.96635703 | 0.53444319 |
| hsa-miR-642_st | 4.231068 | 0.374052 | 4.35733853 | 0.50423828 |
| hsa-miR-29b_st | 11.24211 | 1.292387 | 13.0059592 | 1.27906743 |
| hsa-miR-122_st | 5.148438 | 1.105182 | 4.66908309 | 0.45850611 |
| hsa-miR-645_st | 4.642508 | 0.552498 | 4.52066026 | 0.38427863 |
| hsa-miR-646_st | 3.987533 | 0.409226 | 4.11154526 | 0.4253016 |
| hsa-miR-647_st | 5.052799 | 0.488712 | 4.99012362 | 0.43526436 |
| hsa-miR-648_st | 9.006868 | 0.579699 | 8.37771057 | 0.57515981 |
| hsa-miR-649_st | 4.139383 | 0.544426 | 4.24262331 | 0.5901298 |
| hsa-miR-182_st | 8.320659 | 1.634839 | 9.80407663 | 1.36412885 |
| hsa-miR-651_st | 3.406728 | 0.404252 | 3.37830027 | 0.51666625 |
| hsa-miR-652_st | 7.762146 | 0.785144 | 7.66090052 | 0.90367213 |
| hsa-miR-653_st | 3.106265 | 0.559105 | 3.28515914 | 0.61782589 |
| hsa-miR-654-3p_st | 4.409858 | 0.545831 | 4.69654436 | 1.06940556 |
| hsa-miR-374b_st | 6.784179 | 1.511822 | 8.42756078 | 1.26683136 |
| hsa-miR-130a_st | 10.67886 | 1.109648 | 12.0938004 | 1.15952042 |
| hsa-miR-656_st | 3.858076 | 0.518851 | 3.81634589 | 0.48296611 |
| hsa-miR-657_st | 4.309389 | 0.504934 | 4.2909408 | 0.51396433 |
| hsa-miR-658_st | 12.15535 | 0.595876 | 11.5767069 | 0.73000695 |
| hsa-miR-99b_st | 10.84881 | 0.914911 | 12.0797785 | 0.89322913 |
| hsa-miR-660_st | 7.176118 | 1.310156 | 8.24138059 | 1.07761713 |
| hsa-miR-661_st | 7.553739 | 0.606354 | 7.00692283 | 0.69802235 |
| hsa-miR-30e-star_st | 9.394527 | 1.297462 | 10.4470716 | 1.29096623 |
| hsa-miR-663_st | 15.74623 | 0.575719 | 15.5314612 | 0.55544452 |
| hsa-miR-663b_st | 10.56342 | 0.561011 | 9.9782248 | 0.87057747 |
| hsa-miR-664_st | 7.247443 | 1.084276 | 7.67582306 | 0.98085867 |
| hsa-miR-664-star_st | 10.07695 | 0.769061 | 9.73410892 | 0.76943364 |
| hsa-miR-30d_st | 12.15585 | 1.10351 | 13.2755269 | 1.07853256 |
| hsa-miR-668_st | 8.535779 | 0.711298 | 8.18468539 | 0.80589631 |
| hsa-miR-670_st | 5.602474 | 0.579955 | 5.36417669 | 0.65667168 |
| hsa-miR-671-3p_st | 7.096539 | 0.613408 | 7.2800469 | 0.5130224 |
| hsa-miR-671-5p_st | 12.48527 | 0.471147 | 11.8491822 | 0.55184124 |
| hsa-miR-30a-star_st | 9.913628 | 1.242736 | 11.0098384 | 1.15372118 |
| hsa-miR-675-star_st | 5.199538 | 0.604135 | 5.00677869 | 0.5925252 |
| hsa-miR-148b_st | 8.623071 | 1.412245 | 9.65379722 | 1.28674343 |
| hsa-miR-708_st | 7.497768 | 1.371632 | 7.68731642 | 1.56253808 |
| hsa-miR-708-star_st | 4.449526 | 0.520553 | 4.45173345 | 0.47032717 |
| hsa-miR-221-star_st | 5.940993 | 1.523642 | 8.93721975 | 2.01740794 |
| hsa-let-7e_st | 12.19725 | 1.072228 | 13.3554996 | 0.84428223 |
| hsa-miR-7-1-star_st | 5.60317 | 0.975417 | 6.69217779 | 1.01206461 |
| hsa-miR-27b_st | 11.72495 | 1.192107 | 13.252076 | 1.25561694 |
| hsa-miR-34a_st | 8.818429 | 1.31172 | 10.7390344 | 1.25160145 |
| hsa-miR-744_st | 14.60302 | 0.433249 | 14.1035807 | 0.61488927 |
| hsa-miR-744-star_st | 4.898123 | 0.610025 | 5.2174349 | 0.58086512 |
| hsa-miR-92b_st | 8.744673 | 0.991824 | 9.9861944 | 1.2929912 |
| hsa-miR-759_st | 3.383379 | 0.549334 | 3.60670846 | 0.45031657 |
| hsa-miR-760_st | 10.52103 | 0.526829 | 9.95947682 | 0.64569263 |
| hsa-miR-92a_st | 10.71168 | 1.073545 | 12.178659 | 1.13539102 |
| hsa-miR-335_st | 8.231074 | 1.463711 | 9.70199242 | 1.43443428 |
| hsa-miR-96_st | 5.672075 | 1.554382 | 7.25538985 | 1.51745738 |
| hsa-miR-95_st | 7.520835 | 1.412527 | 8.74440166 | 1.22617304 |
| hsa-miR-766_st | 7.460315 | 0.689031 | 7.18215676 | 0.47913116 |
| hsa-miR-767-3p_st | 4.410398 | 0.494527 | 4.44379549 | 0.46789476 |
| hsa-miR-767-5p_st | 4.344981 | 0.397539 | 4.30224829 | 0.42130275 |
| hsa-miR-98_st | 9.330166 | 1.285238 | 10.565619 | 1.05791507 |
| hsa-miR-769-5p_st | 6.971213 | 0.579558 | 7.37596354 | 0.64934443 |
| hsa-miR-770-5p_st | 7.70514 | 0.54362 | 7.59789118 | 0.49565896 |
| hsa-miR-802_st | 3.03639 | 0.539363 | 3.03550295 | 0.62054666 |
| hsa-miR-34b-star_st | 5.220218 | 1.028231 | 7.22548669 | 1.42185667 |
| hsa-miR-581_st | 3.158983 | 0.692331 | 3.14330238 | 0.73416128 |
| hsa-miR-181d_st | 8.660836 | 0.752218 | 10.140338 | 1.09480489 |
| hsa-miR-875-5p_st | 3.028981 | 0.646981 | 3.05823682 | 0.71543368 |
| hsa-miR-876-3p_st | 3.346264 | 0.525021 | 3.20019159 | 0.55352855 |
| hsa-miR-876-5p_st | 2.935535 | 0.698056 | 2.88597988 | 0.71634259 |
| hsa-miR-151-3p_st | 8.495696 | 1.167665 | 10.1550927 | 1.03563183 |
| hsa-miR-877-star_st | 6.275541 | 0.65407 | 5.9610019 | 0.59463689 |
| hsa-miR-885-3p_st | 13.5902 | 0.664036 | 13.0211006 | 0.68358004 |
| hsa-miR-200c_st | 13.05241 | 1.071641 | 14.154493 | 0.716178 |
| hsa-miR-429_st | 5.263312 | 1.29015 | 7.58990141 | 2.13760823 |
| hsa-miR-886-5p_st | 12.66264 | 0.585452 | 12.6829797 | 0.73836371 |
| hsa-miR-224_st | 6.426349 | 1.086518 | 8.24613982 | 1.20572049 |
| hsa-miR-888_st | 3.56635 | 0.3945 | 3.71912215 | 0.66238961 |
| hsa-miR-4321_st | 10.23156 | 0.60839 | 9.46000352 | 0.67790189 |
| hsa-miR-889_st | 3.66653 | 0.391983 | 3.50288756 | 0.49165527 |
| hsa-miR-181b_st | 9.800986 | 0.859447 | 11.4622963 | 1.08336793 |
| hsa-miR-891a_st | 6.678209 | 0.764227 | 6.67630315 | 0.78421623 |
| hsa-miR-891b_st | 3.892524 | 0.462191 | 4.01530288 | 0.56083733 |
| hsa-miR-892a_st | 4.48396 | 0.655679 | 4.36701033 | 0.78872615 |
| hsa-miR-892b_st | 4.607657 | 0.544555 | 4.77421859 | 0.52276112 |
| hsa-miR-9_st | 3.134078 | 0.653622 | 3.07933128 | 0.51679944 |
| hsa-miR-222_st | 11.22663 | 1.574958 | 14.2099299 | 1.69655332 |
| hsa-miR-141_st | 8.911893 | 1.320621 | 10.6351303 | 1.12192034 |
| hsa-miR-221_st | 11.30484 | 1.647249 | 14.3068888 | 1.57595605 |
| hsa-miR-924_st | 3.920473 | 0.377516 | 4.13217675 | 0.46480255 |
| hsa-miR-181a_st | 11.42016 | 1.036208 | 13.1392992 | 1.19855222 |
| hsa-miR-181c_st | 8.120238 | 1.315791 | 9.88962567 | 1.30046013 |
| hsa-miR-4324_st | 9.881174 | 1.104873 | 11.350197 | 1.29346178 |
| hsa-miR-125a-5p_st | 13.23993 | 1.232642 | 14.5851589 | 1.01534821 |
| hsa-miR-92b-star_st | 14.88035 | 0.46156 | 14.4377746 | 0.54830917 |
| hsa-miR-3195_st | 13.39169 | 0.575352 | 12.8077476 | 0.68159562 |
| hsa-miR-3137_st | 11.65405 | 0.508726 | 10.8570315 | 0.71462821 |
| hsa-miR-934_st | 5.412581 | 0.438068 | 5.39949302 | 0.53710248 |
| hsa-miR-935_st | 4.901166 | 0.550649 | 4.92593139 | 0.63221158 |
| hsa-miR-551b_st | 3.885523 | 0.821241 | 6.71087698 | 2.41243655 |
| hsa-miR-937_st | 5.57512 | 0.577337 | 5.42869087 | 0.7581966 |
| hsa-miR-938_st | 4.939281 | 0.568237 | 4.74006421 | 0.54321864 |
| hsa-miR-939_st | 13.47753 | 0.461945 | 12.8969978 | 0.56679539 |
| hsa-miR-7-2-star_st | 6.700273 | 1.641495 | 5.80045668 | 1.35019357 |
| hsa-miR-146b-5p_st | 8.970762 | 1.795705 | 13.8870864 | 2.49335522 |
| hsa-miR-941_st | 6.047517 | 0.451351 | 5.96755628 | 0.5282621 |
| hsa-miR-942_st | 3.251245 | 0.463577 | 3.41227636 | 0.50774674 |
| hsa-miR-200a_st | 8.98475 | 1.621996 | 11.0555869 | 2.46346642 |
| hsa-miR-944_st | 3.507183 | 0.582631 | 3.79188423 | 0.54955929 |
| hsa-miR-7_st | 8.560339 | 2.695633 | 7.03280562 | 2.50188996 |
| hsa-miR-375_st | 5.421223 | 0.899364 | 8.71765043 | 2.24799369 |
| hsa-miR-96-star_st | 4.453053 | 0.415636 | 4.41441722 | 0.51430937 |
| hsa-miR-424_st | 7.222357 | 1.839925 | 10.0261894 | 1.72948464 |
| hsa-miR-181a-2-star_st | 6.923355 | 1.163371 | 9.81057879 | 1.42816192 |
| hsa-miR-200b_st | 10.31959 | 1.609665 | 12.3585251 | 2.10861116 |
| hsa-miR-146b-3p_st | 4.555994 | 0.540995 | 8.35448682 | 2.75219474 |
| hsa-miR-99b-star_st | 7.760881 | 0.529145 | 7.9866775 | 0.58771086 |
| hsa-miR-9-star_st | 4.198891 | 0.705891 | 4.13911921 | 0.6703382 |

| | Mean | STDEV | Mean | STDEV |
|---|---|---|---|---|
| Sample Name | Hashimoto's | Hashimoto's | FA | FA |
| hsa-miR-150_st | 12.59297681 | 1.493927192 | 9.680561633 | 1.976770319 |
| hsa-let-7a-2-star_st | 4.368898212 | 0.58069382 | 4.476188045 | 0.624028323 |
| hsa-let-7a-star_st | 3.230338439 | 0.627702949 | 3.474234511 | 0.609768215 |
| hsa-miR-513b_st | 11.54315174 | 0.391133362 | 11.10124324 | 0.593154903 |
| hsa-let-7b-star_st | 7.120969452 | 0.504473131 | 7.322926001 | 0.67786054 |
| hsa-miR-593-star_st | 10.13906651 | 0.609117945 | 9.502495992 | 0.811272937 |
| hsa-let-7c-star_st | 5.325940555 | 0.429990487 | 4.865061648 | 0.432286831 |
| hsa-miR-516a-5p_st | 7.848046402 | 0.767276008 | 7.022663975 | 1.115534602 |
| hsa-miR-142-5p_st | 7.968170255 | 1.615030278 | 5.172063056 | 1.912309791 |
| hsa-miR-650_st | 11.77476775 | 0.516748264 | 10.83037117 | 0.555237967 |
| hsa-miR-513c_st | 8.688858513 | 0.648213699 | 8.152700133 | 0.665420531 |
| hsa-miR-761_st | 8.973965633 | 0.599722764 | 8.11475894 | 0.979308734 |
| hsa-let-7f-1-star_st | 5.411462812 | 0.286922672 | 5.208556663 | 0.678998313 |
| hsa-miR-513a-5p_st | 12.85060904 | 0.240141221 | 12.58166042 | 0.557192603 |
| hsa-miR-654-5p_st | 11.68548225 | 0.595803375 | 11.33565226 | 0.835509136 |
| hsa-let-7g-star_st | 4.956051648 | 0.444466323 | 4.593821717 | 0.279972564 |
| hsa-miR-3163_st | 9.374377934 | 0.442891376 | 8.677252722 | 0.648121653 |
| hsa-let-7i-star_st | 6.089701464 | 0.836709695 | 5.905784911 | 0.564078308 |
| hsa-miR-3165_st | 8.199654367 | 0.550486182 | 7.624753964 | 1.012249009 |
| hsa-miR-3194_st | 11.81088772 | 0.269349412 | 11.45806614 | 0.538496495 |
| hsa-miR-100-star_st | 3.089716063 | 0.451174984 | 3.386335228 | 0.333268402 |
| hsa-miR-4251_st | 7.398130285 | 0.477670357 | 6.948129083 | 0.561313199 |
| hsa-miR-101-star_st | 3.918965924 | 0.596644495 | 3.934116088 | 0.381903628 |
| hsa-miR-922_st | 7.070930052 | 0.523415536 | 6.187529739 | 0.559117884 |
| hsa-miR-103-2-star_st | 5.687758948 | 0.525902446 | 5.269101924 | 0.857773044 |
| hsa-miR-155_st | 11.0042425 | 1.166970695 | 9.000990079 | 1.40128148 |
| hsa-miR-105_st | 3.719719058 | 0.626549171 | 3.990380659 | 0.60015099 |
| hsa-miR-127-5p_st | 8.252086589 | 0.527577375 | 7.602931401 | 0.876725045 |
| hsa-miR-1286_st | 10.13222399 | 0.400816059 | 9.626796635 | 0.519182431 |
| hsa-miR-106a-star_st | 3.332232776 | 0.582368462 | 3.621524088 | 0.493800853 |
| hsa-miR-890_st | 7.348422194 | 0.166758269 | 6.44168761 | 0.987851216 |
| hsa-miR-3126-5p_st | 12.87533671 | 0.357600541 | 12.60389554 | 0.509278546 |
| hsa-miR-489_st | 10.45197691 | 0.358268983 | 10.20829983 | 0.353880515 |
| hsa-miR-10a_st | 8.085912925 | 1.183035434 | 7.96490683 | 2.131207469 |
| hsa-miR-10a-star_st | 5.234263177 | 0.867654912 | 4.940245214 | 1.385190067 |
| hsa-miR-10b_st | 9.044114772 | 1.593743563 | 8.777033758 | 1.961390255 |
| hsa-miR-10b-star_st | 5.335873445 | 0.90502076 | 4.681033937 | 0.373355805 |
| hsa-miR-1178_st | 4.735058152 | 0.522051214 | 4.644716321 | 0.62266766 |
| hsa-miR-1179_st | 3.28332617 | 0.385649074 | 3.351393736 | 0.398049605 |
| hsa-miR-1180_st | 9.237115409 | 0.408395509 | 9.361441797 | 0.459159701 |
| hsa-miR-1181_st | 10.04835518 | 0.79245699 | 9.59940679 | 0.579868118 |
| hsa-miR-3138_st | 11.57390612 | 0.415718555 | 11.37410469 | 0.447760147 |
| hsa-miR-220c_st | 7.280060012 | 0.736851631 | 6.423512263 | 0.609959868 |
| hsa-miR-510_st | 9.614357485 | 0.509943879 | 9.126980608 | 0.527992185 |
| hsa-miR-1185_st | 2.888069184 | 0.677903515 | 3.057689402 | 0.550864652 |
| hsa-miR-1193_st | 10.55368838 | 0.316077687 | 10.44077984 | 0.305635098 |
| hsa-miR-1197_st | 3.414038737 | 0.282664296 | 3.788145088 | 0.494372803 |
| hsa-miR-1200_st | 4.700763306 | 0.447839397 | 4.491163572 | 0.364473866 |
| hsa-miR-1201_st | 8.25790029 | 1.392973834 | 9.18167434 | 0.918640358 |
| hsa-miR-1202_st | 13.66139782 | 0.434937139 | 13.8257535 | 0.362138808 |
| hsa-miR-30c-1-star_st | 10.93876915 | 0.264643927 | 10.57238492 | 0.458980139 |
| hsa-miR-1204_st | 5.670308344 | 0.608380222 | 5.25894626 | 0.409776133 |
| hsa-miR-1205_st | 4.794730246 | 0.737430368 | 4.359698996 | 0.439948585 |
| hsa-miR-1206_st | 2.983425706 | 0.660988291 | 3.05550405 | 0.507036239 |
| hsa-miR-1207-3p_st | 4.333833764 | 0.507873147 | 4.419232999 | 0.489448114 |
| hsa-miR-1207-5p_st | 14.53203489 | 0.446180629 | 14.7608213 | 0.336165461 |
| hsa-miR-1208_st | 10.73440982 | 0.543555646 | 10.70012897 | 0.595024775 |
| hsa-miR-622_st | 9.800256627 | 0.350880697 | 9.152592978 | 0.434713273 |
| hsa-miR-1224-3p_st | 5.732684361 | 0.474099909 | 5.509371139 | 0.617223126 |
| hsa-miR-2116_st | 6.931026822 | 0.786323556 | 6.194705652 | 0.726135255 |
| hsa-miR-1225-3p_st | 6.636019111 | 0.578984226 | 6.318312257 | 0.748651688 |
| hsa-miR-1225-5p_st | 14.37488681 | 0.386359068 | 14.59146794 | 0.374737031 |
| hsa-miR-1226_st | 5.998099992 | 0.834599173 | 5.791750377 | 0.592439803 |
| hsa-miR-30b-star_st | 9.707559367 | 0.322239426 | 9.073672296 | 0.683000471 |
| hsa-miR-1227_st | 4.768560782 | 0.74350072 | 4.266449629 | 0.652925114 |
| hsa-miR-1295_st | 7.354523523 | 0.728007659 | 6.733871719 | 0.500940475 |
| hsa-miR-1228-star_st | 15.15013328 | 0.541470856 | 15.62258357 | 0.454389481 |
| hsa-miR-20b_st | 8.50969933 | 1.066293132 | 8.174274658 | 1.249956355 |
| hsa-miR-122-star_st | 3.185233427 | 0.496339808 | 3.346739181 | 0.353847192 |
| hsa-miR-1231_st | 14.17961263 | 0.277523393 | 14.44552546 | 0.387514954 |
| hsa-miR-1233_st | 6.391984551 | 0.767675819 | 6.271968899 | 0.641260474 |
| hsa-miR-524-5p_st | 6.212197532 | 0.702362811 | 5.126889059 | 0.552902226 |
| hsa-miR-1236_st | 6.515675705 | 0.801562509 | 6.183042057 | 0.741015432 |
| hsa-miR-873_st | 6.073319591 | 0.634256284 | 5.172440164 | 0.549512343 |
| hsa-miR-1238_st | 7.765798236 | 0.412240941 | 7.450122404 | 0.776413145 |
| hsa-miR-124_st | 9.176328976 | 0.428233551 | 8.965378131 | 0.60788998 |
| hsa-miR-1243_st | 4.533701111 | 0.379086635 | 4.16677577 | 0.3364925 |
| hsa-miR-1244_st | 8.934190528 | 0.386619413 | 8.575725103 | 0.897634818 |
| hsa-miR-1245_st | 4.246888267 | 0.314342383 | 4.353102394 | 0.466647166 |
| hsa-miR-3122_st | 12.24711595 | 0.371340084 | 12.06888306 | 0.393510403 |
| hsa-miR-134_st | 10.40126373 | 0.507817346 | 10.228483 | 0.466799184 |
| hsa-miR-1248_st | 10.55484521 | 0.993460982 | 10.70466949 | 0.709109624 |
| hsa-miR-146a_st | 10.60962667 | 1.413941756 | 8.811680519 | 1.362935849 |
| hsa-miR-124-star_st | 5.578431866 | 0.632357573 | 5.364276181 | 0.493679723 |
| hsa-miR-1250_st | 8.273833152 | 0.414868258 | 8.314038506 | 0.578746121 |
| hsa-miR-764_st | 6.37732292 | 0.665663247 | 5.59851193 | 0.66463599 |
| hsa-miR-1252_st | 3.035802767 | 0.338545453 | 3.305094136 | 0.464714766 |
| hsa-miR-223_st | 10.46963756 | 1.055806345 | 8.822220621 | 1.012539407 |
| hsa-miR-34c-3p_st | 8.927813871 | 0.64812154 | 8.657939527 | 0.45247484 |
| hsa-miR-1255a_st | 5.882307175 | 0.890141108 | 5.238712964 | 0.615677035 |
| hsa-miR-1255b_st | 8.1907117 | 0.568069492 | 8.212159462 | 0.482706522 |
| hsa-miR-1256_st | 3.917322672 | 0.673328424 | 4.131763266 | 0.321101775 |
| hsa-miR-1257_st | 5.709353364 | 0.756112862 | 4.966715253 | 0.514710387 |
| hsa-miR-199b-5p_st | 8.343658774 | 1.254969683 | 5.336057707 | 1.415638547 |
| hsa-miR-1259_st | 2.883538272 | 0.61802562 | 3.389172524 | 0.344666748 |
| hsa-miR-492_st | 9.659036672 | 0.43927716 | 9.285367559 | 0.712033384 |
| hsa-miR-3132_st | 10.07553489 | 0.472397597 | 9.563034795 | 0.515911558 |
| hsa-miR-516b_st | 7.669039231 | 0.558242407 | 7.184266859 | 0.649156952 |
| hsa-miR-4294_st | 9.35610409 | 0.51020708 | 9.163002279 | 0.418010743 |
| hsa-miR-125b-2-star_st | 10.69743717 | 0.727380836 | 10.40610237 | 0.863495011 |
| hsa-miR-126_st | 13.19969088 | 1.096832979 | 13.94999059 | 0.91764977 |
| hsa-miR-198_st | 12.77634684 | 0.338633314 | 12.62223737 | 0.506933074 |
| hsa-miR-3191_st | 12.39681862 | 0.426328549 | 12.07068319 | 0.700461519 |
| hsa-miR-1261_st | 8.276447894 | 0.813656373 | 7.744511992 | 0.728778638 |
| hsa-miR-1262_st | 4.722918795 | 0.849045086 | 4.177070424 | 0.518414362 |
| hsa-miR-1263_st | 4.436536564 | 0.418040333 | 4.433252164 | 0.434659227 |
| hsa-miR-1264_st | 3.744398965 | 0.397266987 | 3.585860887 | 0.477493979 |
| hsa-miR-1265_st | 5.271184052 | 0.43816928 | 5.26475671 | 0.404006476 |
| hsa-miR-2114-star_st | 7.837103552 | 0.217825136 | 7.689056246 | 0.82749395 |
| hsa-miR-1267_st | 4.381714412 | 0.481218707 | 4.275438504 | 0.441180782 |
| hsa-miR-1268_st | 14.21344156 | 0.559063317 | 14.4700937 | 0.426303104 |
| hsa-miR-1269_st | 6.368019696 | 0.76007708 | 5.947273708 | 0.496007227 |
| hsa-miR-509-5p_st | 7.00983017 | 0.586954845 | 6.439205494 | 0.735965986 |
| hsa-miR-1270_st | 6.602742454 | 0.367946086 | 6.595886359 | 0.555917437 |
| hsa-miR-1271_st | 8.043061591 | 0.407052077 | 7.694166252 | 0.456739339 |
| hsa-miR-4303_st | 7.625100646 | 0.543561808 | 7.022933249 | 0.582635656 |
| hsa-miR-1273_st | 10.30986617 | 0.529601404 | 10.29150279 | 0.413549099 |
| hsa-miR-583_st | 8.0530791 | 0.40470098 | 7.353675766 | 0.717208585 |
| hsa-miR-1273d_st | 10.73467487 | 0.723483077 | 10.69191695 | 0.43109043 |
| hsa-miR-127-3p_st | 6.486647828 | 1.049403078 | 5.542823496 | 0.438654252 |
| hsa-miR-522-star_st | 6.871939286 | 0.60086288 | 6.39408345 | 0.604156303 |
| hsa-miR-214-star_st | 6.396498347 | 0.906662458 | 5.009781595 | 0.554858801 |
| hsa-miR-184_st | 8.797700636 | 0.389805337 | 8.496907957 | 0.754619695 |
| hsa-miR-614_st | 7.061998442 | 0.669870691 | 6.455119527 | 0.740154509 |
| hsa-miR-526b_st | 6.410725808 | 0.697094321 | 5.733628495 | 0.47375245 |
| hsa-miR-1277_st | 3.143089107 | 0.62005871 | 3.414935703 | 0.385892511 |
| hsa-miR-1278_st | 3.751734251 | 0.323889102 | 3.898132752 | 0.415359342 |
| hsa-miR-1279_st | 3.552121651 | 0.478014165 | 3.661172022 | 0.465986492 |
| hsa-miR-409-3p_st | 7.425641825 | 0.445729154 | 6.17878187 | 0.627588767 |
| hsa-miR-199a-5p_st | 10.52535891 | 1.02828183 | 9.101313876 | 1.097608807 |
| hsa-miR-1281_st | 10.33687103 | 0.785432718 | 10.14125631 | 0.693969679 |
| hsa-miR-1282_st | 4.767649378 | 0.718651091 | 4.262204616 | 0.491628748 |
| hsa-miR-33b-star_st | 7.949574391 | 0.341649439 | 7.476900493 | 0.380016722 |
| hsa-miR-1284_st | 5.704228814 | 0.927946095 | 5.182955295 | 0.64118471 |
| hsa-miR-1253_st | 5.812484515 | 0.554153423 | 4.909346903 | 0.585406878 |
| hsa-miR-584_st | 8.369329563 | 0.497670902 | 8.047552575 | 0.629095445 |
| hsa-miR-1287_st | 6.315226468 | 0.532265223 | 6.133798895 | 0.569991546 |
| hsa-miR-15a_st | 9.912067996 | 1.275736905 | 9.897536489 | 0.958282252 |
| hsa-miR-1289_st | 4.826260212 | 0.805293495 | 4.752716934 | 0.442162917 |
| hsa-miR-1290_st | 10.60530785 | 0.390803131 | 10.62808635 | 0.606253347 |
| hsa-miR-2117_st | 4.856708502 | 0.405835923 | 4.269146611 | 0.61583609 |
| hsa-miR-1292_st | 12.99625987 | 0.495604374 | 12.95641907 | 0.540677535 |
| hsa-miR-1293_st | 7.691813732 | 0.437475938 | 7.606666682 | 0.50661088 |
| hsa-miR-129-3p_st | 3.90440647 | 0.436148719 | 4.06977514 | 0.630690643 |
| hsa-miR-520d-5p_st | 7.670459263 | 0.478581168 | 7.149002313 | 0.540720904 |
| hsa-miR-526a_st | 7.501921285 | 0.274065712 | 6.963728794 | 0.608794781 |
| hsa-miR-129-5p_st | 11.76351963 | 0.587185474 | 11.90263263 | 0.468193022 |
| hsa-miR-1254_st | 12.41669108 | 0.52416756 | 12.18536935 | 0.534266367 |
| hsa-miR-1297_st | 3.138479539 | 0.681752217 | 3.336969236 | 0.426451496 |
| hsa-miR-1298_st | 5.482527022 | 0.815290463 | 4.8686209 | 0.615009965 |
| hsa-miR-1299_st | 6.116402879 | 0.800090751 | 5.908460219 | 0.75320404 |
| hsa-miR-129-star_st | 4.381119254 | 0.49350199 | 4.352813039 | 0.623043894 |
| hsa-miR-1301_st | 8.77496205 | 0.44138579 | 8.678627805 | 0.431305149 |
| hsa-miR-19b_st | 9.601395956 | 1.054203825 | 9.459252981 | 1.066373738 |
| hsa-miR-1303_st | 10.51422819 | 0.520783595 | 10.54876598 | 0.492505832 |
| hsa-miR-1304_st | 7.68966199 | 0.728441986 | 7.866726186 | 0.580464672 |
| hsa-miR-1305_st | 4.543575033 | 1.043303288 | 4.258905443 | 0.464888554 |
| hsa-miR-550_st | 12.55025564 | 0.320714865 | 12.3091211 | 0.486426801 |
| hsa-miR-1307_st | 13.74868831 | 0.518514895 | 13.92452954 | 0.513942464 |
| hsa-miR-1308_st | 14.87028449 | 0.432181686 | 15.30710908 | 0.613766636 |
| hsa-miR-4291_st | 7.783940925 | 0.237855524 | 6.998362959 | 1.068012502 |
| hsa-miR-130a-star_st | 4.007375279 | 0.379362456 | 3.912156435 | 0.534217528 |
| hsa-miR-130b_st | 8.162425028 | 0.468649094 | 7.926300994 | 0.615463497 |
| hsa-miR-130b-star_st | 4.946632361 | 0.424839466 | 4.643847022 | 0.614682309 |
| hsa-miR-921_st | 12.24142875 | 0.445230801 | 12.058672 | 0.34431672 |
| hsa-miR-520a-5p_st | 5.63576188 | 0.463917048 | 4.743103487 | 0.393471908 |
| hsa-miR-711_st | 12.53591279 | 0.398241208 | 12.27387907 | 0.594617387 |
| hsa-miR-1323_st | 8.865654215 | 0.612261565 | 8.844227472 | 0.308464882 |
| hsa-miR-1324_st | 3.438783323 | 0.493367472 | 3.575820473 | 0.52933928 |
| hsa-miR-490-3p_st | 6.806239614 | 0.624042546 | 6.100658922 | 0.489402155 |
| hsa-miR-718_st | 9.324827448 | 0.508420404 | 8.948201663 | 0.691448935 |
| hsa-miR-133b_st | 5.220598991 | 0.637760936 | 5.258255881 | 0.700576168 |
| hsa-miR-431-star_st | 7.109649403 | 0.251983413 | 6.590184438 | 0.530551173 |
| hsa-miR-552_st | 6.356368575 | 0.647654248 | 6.066952382 | 0.720277894 |
| hsa-miR-28-3p_st | 7.281992787 | 0.823908522 | 6.895019688 | 0.957579816 |
| hsa-miR-4265_st | 8.404015205 | 0.435495126 | 8.32715757 | 0.508674201 |
| hsa-miR-643_st | 4.779738854 | 0.426969475 | 4.201678651 | 0.743058953 |
| hsa-miR-136_st | 2.890184532 | 0.558213862 | 3.162133428 | 0.457575376 |
| hsa-miR-136-star_st | 3.216338341 | 0.496628876 | 3.417351586 | 0.391138828 |
| hsa-miR-137_st | 3.220344459 | 0.416991633 | 3.634790107 | 0.363085743 |
| hsa-miR-138_st | 9.51379606 | 1.086567709 | 10.67195657 | 0.800093316 |
| hsa-miR-138-1-star_st | 8.489513565 | 0.538260572 | 8.588418344 | 0.482949732 |
| hsa-miR-138-2-star_st | 5.190108811 | 0.249678662 | 5.332668728 | 0.520164192 |
| hsa-miR-139-3p_st | 8.322152123 | 0.594265758 | 8.01035086 | 0.641212816 |
| hsa-miR-139-5p_st | 7.037960775 | 1.448294822 | 8.209362372 | 0.78676886 |
| hsa-miR-140-3p_st | 11.01194322 | 0.563139461 | 10.73342944 | 0.569273694 |
| hsa-miR-140-5p_st | 6.395775207 | 1.321575874 | 5.945129065 | 0.866211636 |
| hsa-miR-381_st | 7.04502034 | 0.625356042 | 6.231692308 | 0.555481808 |
| hsa-miR-141-star_st | 3.733303263 | 0.509729428 | 3.812289122 | 0.444263688 |
| hsa-miR-17-star_st | 6.448350563 | 0.572822677 | 5.805072541 | 0.537873209 |
| hsa-miR-4300_st | 8.613800226 | 0.466243937 | 7.958352121 | 0.680555144 |
| hsa-miR-143_st | 11.80029015 | 1.088424797 | 12.27332708 | 0.943395752 |
| hsa-miR-143-star_st | 5.979569854 | 0.808577228 | 6.684964249 | 0.807284112 |
| hsa-miR-144_st | 3.13603898 | 0.470668665 | 3.411842382 | 0.413331828 |
| hsa-miR-3198_st | 10.42228431 | 0.273030853 | 10.20121318 | 0.437168827 |
| hsa-miR-145_st | 12.34424801 | 1.08868289 | 13.19838962 | 0.712228298 |
| hsa-miR-145-star_st | 5.413067017 | 0.960988521 | 5.679842498 | 1.341225225 |
| hsa-miR-1468_st | 6.181761372 | 0.978342561 | 6.010161668 | 0.852322405 |
| hsa-miR-1469_st | 15.29018598 | 0.572766323 | 15.98482591 | 0.454211488 |
| hsa-miR-1973_st | 15.0382681 | 0.579746007 | 15.49123238 | 0.600947367 |
| hsa-miR-3154_st | 12.02055305 | 0.452014401 | 11.73806747 | 0.278056041 |
| hsa-miR-148a-star_st | 6.23799973 | 0.4016161 | 5.541215715 | 0.652361835 |
| hsa-miR-92a-1-star_st | 8.570503065 | 0.578042905 | 8.159637583 | 0.337849176 |
| hsa-miR-147_st | 4.284393717 | 0.452499164 | 4.270340607 | 0.389067898 |
| hsa-miR-194-star_st | 9.159880339 | 0.598652563 | 8.832984032 | 0.632600779 |
| hsa-miR-1471_st | 11.3878725 | 0.859950302 | 11.1255924 | 0.525165298 |
| hsa-miR-147b_st | 4.85865749 | 0.387316736 | 4.517759129 | 0.453339436 |
| hsa-miR-148a_st | 10.97158612 | 0.949944088 | 11.63704493 | 1.024027428 |
| hsa-miR-4299_st | 13.81659249 | 0.248550142 | 13.91344208 | 0.446050473 |
| hsa-miR-135a-star_st | 13.50517178 | 0.516465855 | 13.3464063 | 0.420961133 |
| hsa-miR-612_st | 12.41954996 | 0.525182181 | 12.07050245 | 0.48592747 |
| hsa-miR-1321_st | 8.092763529 | 0.419292019 | 7.69663273 | 0.495139777 |
| hsa-miR-149-star_st | 15.28597629 | 0.573333644 | 15.91547129 | 0.414391933 |
| hsa-miR-604_st | 9.778796173 | 0.295523916 | 9.682478741 | 0.402774636 |
| hsa-miR-150-star_st | 14.22966314 | 0.379879756 | 14.42183934 | 0.392074932 |
| hsa-miR-125b-1-star_st | 12.30506459 | 0.398136879 | 11.94795678 | 0.562528852 |
| hsa-miR-1183_st | 10.63456706 | 0.31879299 | 10.43869103 | 0.314652122 |
| hsa-miR-152_st | 9.348215417 | 1.234769248 | 10.42296212 | 0.911316914 |
| hsa-miR-153_st | 3.431151403 | 0.464262852 | 3.776103249 | 0.473040479 |
| hsa-miR-1537_st | 3.365761206 | 0.511408159 | 3.446122447 | 0.529613126 |
| hsa-miR-1538_st | 7.883596862 | 0.343325953 | 8.077112647 | 0.562697067 |
| hsa-miR-1539_st | 5.821310732 | 0.528544205 | 5.7348157 | 0.37639766 |
| hsa-miR-154_st | 5.017602473 | 0.764620653 | 4.645635018 | 0.545623289 |
| hsa-miR-154-star_st | 5.468651709 | 0.779266185 | 4.614946983 | 0.566617555 |
| hsa-miR-432-star_st | 5.631413585 | 0.724099146 | 4.706952649 | 0.50121821 |
| hsa-miR-155-star_st | 3.2518343 | 0.450096824 | 3.697576216 | 0.332710499 |
| hsa-miR-298_st | 11.27096321 | 0.42345003 | 10.99572539 | 0.447493668 |
| hsa-miR-15a-star_st | 5.25478601 | 0.862019686 | 5.026365894 | 0.809239072 |
| hsa-miR-2110_st | 11.39380778 | 0.289813785 | 11.19069899 | 0.740794188 |
| hsa-miR-15b-star_st | 3.890166979 | 0.744161027 | 3.74929862 | 0.622325845 |
| hsa-miR-1266_st | 9.96625722 | 0.457454288 | 9.764862447 | 0.498449287 |
| hsa-miR-16-1-star_st | 4.636567414 | 0.394399293 | 4.616639694 | 0.361503493 |
| hsa-miR-16-2-star_st | 4.774362535 | 0.709804814 | 4.801322524 | 0.481349881 |
| hsa-miR-490-5p_st | 8.045061147 | 0.472448416 | 7.759574176 | 0.487181889 |
| hsa-miR-19a_st | 5.940003581 | 1.207340569 | 5.591131621 | 0.923369463 |
| hsa-miR-3192_st | 9.388884824 | 0.377543385 | 9.111902073 | 0.448730631 |
| hsa-miR-3155_st | 8.413696078 | 0.338867414 | 7.837110229 | 0.427101101 |
| hsa-miR-187-star_st | 11.36303714 | 0.382765003 | 11.36127305 | 0.348406872 |
| hsa-miR-16_st | 13.17782644 | 0.856276508 | 13.13051481 | 0.675319087 |
| hsa-miR-936_st | 11.41059269 | 0.56058769 | 11.11624365 | 0.255196152 |
| hsa-miR-181c-star_st | 7.663847447 | 0.460229201 | 7.458598744 | 0.569050369 |
| hsa-miR-379_st | 6.562358207 | 0.310458882 | 5.72442109 | 0.613897801 |
| hsa-miR-370_st | 11.53487494 | 0.541065532 | 11.58933444 | 0.466867693 |
| hsa-miR-541_st | 9.007309072 | 0.363139935 | 8.791512743 | 0.503092957 |
| hsa-miR-518c-star_st | 9.00865515 | 0.491677011 | 8.70984069 | 0.374279069 |
| hsa-miR-487b_st | 8.844536782 | 1.085199516 | 8.959238316 | 0.906423418 |
| hsa-miR-182-star_st | 3.274267497 | 0.403099836 | 3.526936776 | 0.285724948 |
| hsa-miR-3121_st | 8.403770303 | 0.511260865 | 8.192220607 | 0.635880412 |
| hsa-miR-3190-5p_st | 10.64526171 | 0.29222142 | 10.48540666 | 0.435911062 |
| hsa-miR-1203_st | 9.455249589 | 0.352757008 | 9.275954018 | 0.403888763 |
| hsa-miR-185_st | 9.377498703 | 0.46565094 | 9.36294786 | 0.703206029 |
| hsa-miR-185-star_st | 9.731613461 | 0.486840207 | 9.548973138 | 0.64453104 |
| hsa-miR-4317_st | 10.39929652 | 0.383841285 | 10.22601623 | 0.539247807 |
| hsa-miR-186-star_st | 3.167456881 | 0.525039539 | 3.104754006 | 0.553441475 |
| hsa-miR-187_st | 5.855320542 | 0.555911803 | 5.303844034 | 0.891042561 |
| hsa-miR-4257_st | 12.76496141 | 0.479207718 | 12.75927404 | 0.332969343 |
| hsa-miR-188-3p_st | 5.026897235 | 0.739539037 | 4.61630837 | 0.521490701 |
| hsa-miR-1184_st | 5.813593337 | 0.503516567 | 5.176397611 | 0.58494121 |
| hsa-miR-18a_st | 5.058342252 | 0.330475665 | 4.607061934 | 0.759798443 |
| hsa-miR-18a-star_st | 5.57821253 | 0.75759913 | 5.226058381 | 0.64242214 |
| hsa-miR-3140_st | 5.971973814 | 0.655518682 | 5.047745257 | 0.438290744 |
| hsa-miR-18b-star_st | 4.695490903 | 0.738311834 | 4.385397265 | 0.256041553 |
| hsa-miR-190_st | 2.640697035 | 0.532441154 | 3.112156432 | 0.727952426 |
| hsa-miR-601_st | 6.638540853 | 0.325898542 | 6.528271706 | 0.851792055 |
| hsa-miR-1909_st | 14.69273882 | 0.346627258 | 14.75048598 | 0.451076953 |
| hsa-miR-342-5p_st | 6.359989959 | 0.74141318 | 5.388330858 | 0.760959902 |
| hsa-miR-575_st | 13.76414259 | 0.3448865 | 13.9405819 | 0.455774818 |
| hsa-miR-885-5p_st | 7.305481855 | 0.170038563 | 7.048830458 | 0.746833837 |
| hsa-miR-300_st | 6.362645721 | 0.449742117 | 5.970357 | 0.59994688 |
| hsa-miR-1911_st | 4.600373861 | 0.802898143 | 4.193433417 | 0.414958019 |
| hsa-miR-1911-star_st | 6.047802634 | 0.484632737 | 5.380550732 | 0.722980269 |
| hsa-miR-1912_st | 4.903858015 | 0.494711455 | 4.624507585 | 0.45181846 |
| hsa-miR-4266_st | 7.203928071 | 0.204192164 | 6.719455231 | 0.650740081 |
| hsa-miR-1914_st | 6.003719432 | 0.525418265 | 5.841247451 | 0.596677388 |
| hsa-miR-3190-3p_st | 11.22712183 | 0.351888979 | 11.28009251 | 0.316823907 |
| hsa-miR-618_st | 4.550836067 | 0.476917075 | 3.941733546 | 0.326032533 |
| hsa-miR-1276_st | 7.63194633 | 0.495361224 | 7.348842284 | 0.649327547 |
| hsa-miR-191-star_st | 7.025457938 | 0.581820547 | 6.783103247 | 0.851047438 |
| hsa-miR-1258_st | 5.612272209 | 0.323717986 | 5.135981625 | 0.504250726 |
| hsa-miR-192-star_st | 3.84201051 | 0.462954871 | 4.011675706 | 0.501862631 |
| hsa-miR-193a-3p_st | 7.568963535 | 0.622273055 | 7.642839137 | 1.301010916 |
| hsa-miR-193a-5p_st | 12.72807685 | 0.337471845 | 12.98096904 | 0.385582348 |
| hsa-miR-3157_st | 5.759137402 | 0.437829308 | 5.330767541 | 0.273398309 |
| hsa-miR-193b-star_st | 11.29302541 | 0.41887514 | 11.20686901 | 0.402420576 |
| hsa-miR-194_st | 6.651991732 | 1.530259222 | 7.170005188 | 1.231659284 |
| hsa-miR-4307_st | 4.457446636 | 0.401460385 | 3.776068137 | 0.508536222 |
| hsa-miR-195_st | 10.85557618 | 0.789325673 | 11.64324192 | 0.845762285 |
| hsa-miR-610_st | 6.343888877 | 0.451680787 | 5.688924457 | 0.521109134 |
| hsa-miR-196a_st | 4.660586118 | 0.988345407 | 4.669550936 | 0.703856682 |
| hsa-miR-196a-star_st | 5.984980565 | 0.668019133 | 6.435395573 | 0.850318501 |
| hsa-miR-196b_st | 3.786350854 | 0.357301044 | 4.194671926 | 0.486403292 |
| hsa-miR-196b-star_st | 6.107369945 | 0.296279163 | 5.892434246 | 0.499977315 |
| hsa-let-7f-2-star_st | 4.325184829 | 0.434691288 | 3.694520537 | 0.392398101 |
| hsa-miR-1972_st | 12.60833799 | 0.658594338 | 12.58168553 | 0.512639748 |
| hsa-miR-3184_st | 10.59061984 | 0.335389535 | 10.29022648 | 0.592093543 |
| hsa-miR-523-star_st | 6.769203429 | 0.380203317 | 6.494685715 | 0.731056707 |
| hsa-miR-1976_st | 7.350990606 | 0.736126706 | 7.011195428 | 0.791236192 |
| hsa-miR-625-star_st | 5.070569401 | 0.232981354 | 4.356170861 | 0.510997662 |
| hsa-miR-587_st | 5.307002914 | 0.430236466 | 5.078528663 | 0.450415921 |
| hsa-miR-199a-3p_st | 11.68120935 | 1.06998233 | 10.4840672 | 0.927152967 |
| hsa-miR-1910_st | 9.269185002 | 0.474996479 | 8.881228569 | 0.317932991 |
| hsa-miR-199b-3p_st | 11.53994739 | 1.122112158 | 10.712705 | 0.829555674 |
| hsa-miR-639_st | 9.378550407 | 0.496927687 | 9.187536885 | 0.955381778 |
| hsa-miR-1909-star_st | 9.235986566 | 0.09537851 | 8.7244257 | 0.489301447 |
| hsa-miR-19a-star_st | 2.976220386 | 0.493263466 | 3.303514396 | 0.63058184 |
| hsa-miR-29c-star_st | 6.853146715 | 1.469213543 | 7.740637686 | 0.688003874 |
| hsa-miR-940_st | 9.212092664 | 0.474834334 | 8.917964765 | 0.488480576 |
| hsa-miR-19b-2-star_st | 3.059814007 | 0.341694828 | 3.005228387 | 0.581801252 |
| hsa-miR-4269_st | 8.981503238 | 0.344938129 | 8.616971493 | 0.527530453 |
| hsa-miR-200a-star_st | 6.916820447 | 0.463657355 | 6.638609214 | 0.677381499 |
| hsa-miR-1182_st | 10.77634451 | 0.371307879 | 10.66976935 | 0.325362897 |
| hsa-miR-518f-star_st | 7.287741756 | 0.138252405 | 6.83490537 | 0.543979698 |
| hsa-miR-92a-2-star_st | 9.405290013 | 0.301702649 | 9.179324331 | 0.569641345 |
| hsa-miR-200c-star_st | 4.208917552 | 0.686741366 | 4.405990797 | 0.674489036 |
| hsa-miR-202_st | 9.627548021 | 0.746984887 | 9.31472474 | 0.427059353 |
| hsa-miR-202-star_st | 2.578214413 | 0.607423663 | 3.093716672 | 0.448813034 |
| hsa-miR-1914-star_st | 13.51365221 | 0.293210118 | 13.43599804 | 0.540965097 |
| hsa-miR-204_st | 6.545023442 | 2.308105147 | 8.176927693 | 2.488648826 |
| hsa-miR-665_st | 13.24658039 | 0.292473067 | 12.95615237 | 0.430695782 |
| hsa-miR-2052_st | 2.760075146 | 0.555404176 | 3.1531863 | 0.699163894 |
| hsa-miR-2053_st | 3.346415614 | 0.671853663 | 3.718443561 | 0.515446155 |
| hsa-miR-2054_st | 2.354724102 | 1.044023463 | 2.917363703 | 0.852013343 |
| hsa-miR-205-star_st | 5.505860754 | 0.611554474 | 5.494065975 | 0.725187215 |
| hsa-miR-206_st | 6.814145472 | 0.513806714 | 6.197200523 | 0.48880875 |
| hsa-miR-208a_st | 3.903928092 | 0.276030838 | 3.989947251 | 0.738610505 |
| hsa-miR-208b_st | 3.926105847 | 0.306093544 | 4.01584638 | 0.718944553 |
| hsa-miR-18b_st | 4.668858511 | 0.28665112 | 4.165489246 | 0.338448889 |
| hsa-miR-20a-star_st | 4.443416371 | 1.376381674 | 4.145007003 | 0.492730964 |
| hsa-miR-3181_st | 8.974298368 | 0.231934232 | 8.872343368 | 0.490970641 |
| hsa-miR-615-5p_st | 11.56240824 | 0.268978302 | 11.15840105 | 0.696950903 |
| hsa-miR-4259_st | 11.15870007 | 0.450103014 | 11.07611358 | 0.429458326 |
| hsa-miR-210_st | 12.94487154 | 0.508229734 | 13.03031693 | 0.434108253 |
| hsa-miR-211_st | 5.407777645 | 0.845988511 | 5.20158111 | 0.64253476 |
| hsa-miR-887_st | 9.765599704 | 0.42698835 | 9.449489036 | 0.426397089 |
| hsa-miR-2113_st | 5.801335877 | 0.835950895 | 5.041057024 | 0.761862192 |
| hsa-miR-2114_st | 4.696035322 | 0.796251249 | 4.176838826 | 0.346821758 |
| hsa-miR-943_st | 7.694423938 | 0.452350592 | 7.641173266 | 0.760082538 |
| hsa-miR-2115_st | 3.201588126 | 0.418695884 | 3.329121811 | 0.495899106 |
| hsa-miR-2115-star_st | 10.48023923 | 0.634753156 | 11.04554668 | 1.009084519 |
| hsa-miR-183-star_st | 7.818580682 | 0.318013874 | 7.619040909 | 0.596060563 |
| hsa-miR-2116-star_st | 4.723772704 | 0.313965102 | 4.241287031 | 0.881558633 |
| hsa-miR-920_st | 10.74457322 | 0.369714646 | 10.58578481 | 0.643697709 |
| hsa-miR-212_st | 6.705874851 | 0.334308619 | 6.271027126 | 0.530595347 |
| hsa-miR-214_st | 11.02630929 | 0.509666041 | 10.57168393 | 0.504975226 |
| hsa-miR-1825_st | 7.410962651 | 0.436920996 | 6.839384674 | 0.696972939 |
| hsa-miR-215_st | 6.422174142 | 0.790867758 | 5.741971851 | 0.950378666 |
| hsa-miR-216a_st | 3.863135597 | 0.605866789 | 3.883299785 | 0.374058359 |
| hsa-miR-195-star_st | 6.165872399 | 0.427606025 | 5.607956309 | 0.748938875 |
| hsa-miR-217_st | 3.214248113 | 0.368647406 | 3.450278032 | 0.335836861 |
| hsa-miR-23a-star_st | 11.20395014 | 0.449235222 | 11.06230496 | 0.502263536 |
| hsa-miR-25-star_st | 12.96805596 | 0.273925721 | 12.8288534 | 0.499957547 |
| hsa-miR-218-2-star_st | 4.628488181 | 0.410017353 | 4.331783998 | 0.446951497 |
| hsa-miR-1273c_st | 11.12970939 | 0.059694025 | 11.05626111 | 0.42259596 |
| hsa-miR-219-2-3p_st | 7.386043654 | 0.459281434 | 7.218193955 | 0.506772461 |
| hsa-miR-637_st | 12.2380645 | 0.37534016 | 12.1878469 | 0.511503359 |
| hsa-miR-21-star_st | 7.295021846 | 0.413761578 | 6.600574263 | 0.904685023 |
| hsa-miR-22_st | 10.91315927 | 1.292068644 | 12.0285146 | 0.923234886 |
| hsa-miR-220a_st | 3.326423436 | 0.499844818 | 3.423820136 | 0.464684183 |
| hsa-miR-220b_st | 3.924414178 | 0.822393298 | 3.88892985 | 0.336400611 |
| hsa-miR-219-1-3p_st | 6.120407172 | 0.33281665 | 5.704502402 | 0.403114258 |
| hsa-miR-4316_st | 7.849336193 | 0.384567978 | 7.736813109 | 0.551657609 |
| hsa-miR-877_st | 12.60870085 | 0.370165904 | 12.63107819 | 0.38536824 |
| hsa-miR-636_st | 7.965892976 | 0.184383766 | 7.825257039 | 0.691251909 |
| hsa-miR-3177_st | 12.27032888 | 0.438373728 | 12.26541939 | 0.539604174 |
| hsa-miR-1249_st | 5.462245426 | 0.30738947 | 4.949882595 | 0.6268105 |
| hsa-miR-223-star_st | 4.500080107 | 0.449587235 | 4.102742036 | 0.480348152 |
| hsa-miR-1224-5p_st | 13.73200803 | 0.363227423 | 13.81215448 | 0.213992569 |
| hsa-miR-564_st | 12.58505158 | 0.331378659 | 12.55010663 | 0.391707845 |
| hsa-miR-2276_st | 11.63683935 | 0.550169646 | 11.68901578 | 0.5330301 |
| hsa-miR-2277_st | 7.450432673 | 0.092822088 | 7.397569279 | 0.609053951 |
| hsa-miR-2278_st | 6.595480614 | 0.618968643 | 6.424664461 | 0.621708168 |
| hsa-miR-22-star_st | 6.843601838 | 1.452299576 | 8.270075581 | 1.070902988 |
| hsa-miR-2355_st | 4.64994947 | 0.667242155 | 4.153860831 | 0.475868392 |
| hsa-miR-769-3p_st | 9.811633221 | 0.340092065 | 9.448050583 | 0.508736112 |
| hsa-miR-1913_st | 8.08372926 | 0.323769725 | 7.721164302 | 0.509474388 |
| hsa-miR-1237_st | 7.697861326 | 0.277147366 | 7.346392781 | 0.524149581 |
| hsa-miR-1275_st | 13.85026207 | 0.293408242 | 13.79885195 | 0.355554775 |
| hsa-miR-30a_st | 11.66306692 | 1.333084928 | 12.57025379 | 0.669974915 |
| hsa-miR-20b-star_st | 6.187310909 | 0.203685582 | 5.990653449 | 0.543675506 |
| hsa-miR-24-2-star_st | 6.969663452 | 0.79206723 | 6.037840766 | 0.803139343 |
| hsa-miR-3173_st | 10.144865 | 0.24151213 | 10.0414114 | 0.41142212 |
| hsa-miR-3148_st | 5.938334859 | 0.313997028 | 5.68622722 | 0.598628803 |
| hsa-miR-4271_st | 12.73686094 | 0.306387833 | 12.88325576 | 0.200970738 |
| hsa-miR-659_st | 11.06023921 | 0.377207571 | 10.94202916 | 0.43258648 |
| hsa-miR-26a-2-star_st | 2.50902779 | 0.562748283 | 3.071132329 | 0.718697485 |
| hsa-miR-1228_st | 7.485638214 | 0.225204381 | 7.095711605 | 0.732006376 |
| hsa-miR-26b-star_st | 5.222182122 | 0.762304372 | 5.417197762 | 0.583852434 |
| hsa-miR-3174_st | 7.802803437 | 0.336549564 | 7.686101753 | 0.711722026 |
| hsa-miR-27a-star_st | 6.264972807 | 0.838568284 | 5.442168851 | 0.461898557 |
| hsa-miR-1285_st | 13.00598097 | 0.338693999 | 12.87188152 | 0.314517442 |
| hsa-miR-483-5p_st | 12.18239486 | 0.30638993 | 12.10051282 | 0.249688589 |
| hsa-miR-1306_st | 7.321087756 | 0.300005004 | 7.180460859 | 0.458141661 |
| hsa-miR-431_st | 4.374879212 | 0.214524022 | 3.987032986 | 0.392380707 |
| hsa-miR-765_st | 9.475196781 | 0.27331103 | 9.268111222 | 0.36723301 |
| hsa-miR-2909_st | 3.816773155 | 0.378183045 | 4.097995858 | 0.415540151 |
| hsa-miR-296-3p_st | 11.55649972 | 0.605449361 | 11.48000471 | 0.375333936 |
| hsa-miR-296-5p_st | 4.919553775 | 0.705856868 | 4.441437937 | 0.48574802 |
| hsa-miR-297_st | 5.659283477 | 0.559042855 | 5.316389436 | 0.68730864 |
| hsa-miR-323b-5p_st | 7.899009606 | 0.325763596 | 7.590828184 | 0.70969393 |
| hsa-miR-299-3p_st | 7.91068893 | 0.637259974 | 7.306640606 | 0.810793024 |
| hsa-miR-299-5p_st | 3.940575681 | 0.335669601 | 4.101872632 | 0.344268233 |
| hsa-miR-487a_st | 5.987019981 | 0.69742572 | 5.738385626 | 0.727555371 |
| hsa-miR-29a-star_st | 3.56493965 | 0.231192424 | 3.592211235 | 0.67208154 |
| hsa-miR-625_st | 7.634657094 | 0.173404529 | 6.838309084 | 0.427354793 |
| hsa-miR-611_st | 8.518541752 | 0.300012308 | 8.101312402 | 0.786451505 |
| hsa-miR-3186-3p_st | 9.243000141 | 0.3015793 | 8.858347367 | 0.592675032 |
| hsa-miR-1294_st | 6.1706879 | 0.134830064 | 5.765477705 | 0.498227506 |
| hsa-miR-3147_st | 10.7830605 | 0.333592962 | 10.82864992 | 0.400846036 |
| hsa-miR-4285_st | 11.75144695 | 0.137368992 | 11.75567428 | 0.684749329 |
| hsa-miR-125a-3p_st | 12.00569563 | 0.360055672 | 11.81496195 | 0.39624021 |
| hsa-miR-301b_st | 3.259983602 | 0.450638397 | 3.439428324 | 0.557724067 |
| hsa-miR-302a_st | 3.368846232 | 0.478968678 | 3.477564279 | 0.613201997 |
| hsa-miR-518e-star_st | 6.970699489 | 0.404168862 | 6.504477272 | 0.577117365 |
| hsa-miR-302b_st | 2.597282043 | 0.919406479 | 3.230136027 | 0.629390845 |
| hsa-miR-302b-star_st | 4.215388458 | 0.267642731 | 4.483982668 | 0.375640697 |
| hsa-miR-302c_st | 3.230956033 | 0.564849903 | 2.979927799 | 0.410501244 |
| hsa-miR-302c-star_st | 9.053419999 | 0.481100529 | 9.249888538 | 0.490524816 |
| hsa-miR-302d_st | 3.175695033 | 0.467019518 | 3.321578983 | 0.407216318 |
| hsa-miR-302d-star_st | 8.631883853 | 0.323235615 | 9.045044258 | 0.92045216 |
| hsa-miR-302e_st | 3.816638698 | 0.606233875 | 3.583729011 | 0.555188943 |
| hsa-miR-302f_st | 3.148323675 | 0.332905323 | 3.441831968 | 0.476211155 |
| hsa-miR-3065-3p_st | 5.761935044 | 0.985723987 | 5.105828157 | 0.473042377 |
| hsa-miR-3130-5p_st | 7.471005902 | 0.208931189 | 7.489599573 | 0.594629894 |
| hsa-miR-3074_st | 5.441927309 | 0.499434479 | 5.317937234 | 0.571963105 |
| hsa-miR-1226-star_st | 11.98645622 | 0.248957447 | 11.99819789 | 0.477014517 |
| hsa-miR-3180-5p_st | 7.032591818 | 0.195015496 | 7.000051832 | 0.512388455 |
| hsa-miR-1229_st | 7.132909193 | 0.430107361 | 7.018577284 | 0.410224107 |
| hsa-miR-1272_st | 4.878169992 | 0.276113535 | 4.509970111 | 0.462687047 |
| hsa-miR-30c_st | 12.5672631 | 1.292946599 | 13.94021513 | 0.52999949 |
| hsa-miR-675_st | 13.6063338 | 0.302432994 | 13.4965141 | 0.542711393 |
| hsa-miR-30c-2-star_st | 7.668069071 | 0.630449366 | 7.263663226 | 0.665588994 |
| hsa-miR-1827_st | 4.649087415 | 0.44266365 | 4.414640605 | 0.40455572 |
| hsa-miR-30d-star_st | 3.648343129 | 0.696573446 | 3.728423108 | 0.513629088 |
| hsa-miR-30e_st | 9.307704733 | 1.26066379 | 10.14849389 | 0.992619798 |
| hsa-miR-1915-star_st | 7.434364284 | 0.243492889 | 7.030711072 | 0.731973496 |
| hsa-miR-1322_st | 4.691025546 | 0.234154911 | 4.311379098 | 0.371918202 |
| hsa-miR-3115_st | 3.552953812 | 0.62131637 | 3.543149629 | 0.373326216 |
| hsa-miR-3116_st | 7.580046647 | 0.871614745 | 7.143605467 | 0.521457719 |
| hsa-miR-3117_st | 4.701389042 | 0.446000686 | 4.65215389 | 0.383879527 |
| hsa-miR-3118_st | 3.455323211 | 0.552114285 | 3.831231964 | 0.417708026 |
| hsa-miR-519b-5p_st | 6.386126474 | 0.318796048 | 6.08869309 | 0.542952942 |
| hsa-miR-3120_st | 7.095836313 | 0.794855416 | 6.724013822 | 0.451479382 |
| hsa-miR-520d-3p_st | 5.752904031 | 0.248336318 | 5.558840417 | 0.549552874 |
| hsa-miR-326_st | 6.176684958 | 0.718141814 | 5.824990481 | 0.32232359 |
| hsa-miR-3123_st | 2.823050653 | 0.627208915 | 3.011666614 | 0.708174529 |
| hsa-miR-3124_st | 12.56615148 | 0.345904585 | 12.47282686 | 0.49829628 |
| hsa-miR-3125_st | 7.944418984 | 0.383849872 | 7.55203057 | 0.765066912 |
| hsa-miR-3126-3p_st | 4.920841204 | 0.57823121 | 4.892667168 | 0.519594591 |
| hsa-miR-1296_st | 7.163665411 | 0.275273924 | 6.71151592 | 0.591026522 |
| hsa-miR-3127_st | 9.45291955 | 0.728597094 | 9.479477745 | 0.354084814 |
| hsa-miR-3128_st | 4.089988171 | 0.239894206 | 4.192276261 | 0.385750184 |
| hsa-miR-3129_st | 4.37913296 | 0.291774651 | 4.281724146 | 0.391177044 |
| hsa-miR-3130-3p_st | 9.163500897 | 0.606641183 | 8.862886485 | 0.469689533 |
| hsa-miR-105-star_st | 4.306047532 | 0.276473487 | 3.927099582 | 0.47288221 |
| hsa-miR-3131_st | 12.15605251 | 0.498658861 | 11.9589446 | 0.432296015 |
| hsa-miR-4255_st | 4.485985221 | 0.170317023 | 4.114825097 | 0.356452809 |
| hsa-miR-3133_st | 4.303866677 | 0.586581086 | 4.925889371 | 1.784977189 |
| hsa-miR-3134_st | 3.896287544 | 0.384436651 | 3.575803099 | 0.608940554 |
| hsa-miR-3135_st | 6.997822745 | 0.804121787 | 6.807215094 | 0.471767523 |
| hsa-miR-3136_st | 6.292801128 | 0.511485657 | 6.392019402 | 0.458735583 |
| hsa-miR-19b-1-star_st | 3.943443878 | 0.347183576 | 3.959147049 | 0.32500859 |
| hsa-miR-1247_st | 7.255813785 | 0.235726318 | 7.124789375 | 0.65019808 |
| hsa-miR-3139_st | 4.076816362 | 0.24161553 | 3.90671338 | 0.354161945 |
| hsa-miR-596_st | 6.728786675 | 0.131994596 | 6.425032448 | 0.486581407 |
| hsa-miR-3141_st | 14.31298038 | 0.430497846 | 14.53239362 | 0.422062213 |
| hsa-miR-3142_st | 2.893773803 | 0.693809741 | 3.285104865 | 0.440907613 |
| hsa-miR-1302_st | 3.581719298 | 0.36091295 | 3.810408542 | 0.236689367 |
| hsa-miR-3144-3p_st | 4.971301717 | 1.000198783 | 4.372462998 | 0.400731356 |
| hsa-miR-3144-5p_st | 6.025695932 | 1.051816445 | 5.189687362 | 0.369714383 |
| hsa-miR-3145_st | 3.428459876 | 0.732751368 | 3.489102691 | 0.61173327 |
| hsa-miR-3146_st | 4.920849838 | 0.63733804 | 4.804815077 | 0.447846595 |
| hsa-miR-346_st | 8.546477279 | 0.197421063 | 8.478721929 | 0.517790544 |
| hsa-miR-4256_st | 4.308171864 | 0.248339018 | 4.090035948 | 0.365676689 |
| hsa-miR-3149_st | 4.932551145 | 0.57281601 | 4.587341072 | 0.593502683 |
| hsa-miR-3150_st | 9.974437455 | 0.516556671 | 9.718009019 | 0.482252317 |
| hsa-miR-3151_st | 11.78569845 | 0.372343445 | 11.7102942 | 0.458831102 |
| hsa-miR-1470_st | 5.831666096 | 0.210671054 | 5.518516658 | 0.434967592 |
| hsa-miR-3153_st | 9.76080699 | 0.562439606 | 9.438487878 | 0.562261459 |
| hsa-miR-514_st | 3.080612728 | 0.201089517 | 3.451818868 | 0.383807564 |
| hsa-miR-3175_st | 12.87838752 | 0.102131539 | 13.11896015 | 0.280641082 |
| hsa-miR-3156_st | 10.57231291 | 0.361051687 | 10.4790666 | 0.433171319 |
| hsa-miR-504_st | 5.369207788 | 0.254472427 | 5.643876196 | 0.654856859 |
| hsa-miR-3158_st | 5.199532384 | 0.638158246 | 4.862517098 | 0.792679554 |
| hsa-miR-3159_st | 5.35454995 | 0.520929429 | 4.996293774 | 0.413051303 |
| hsa-miR-3160_st | 6.329029618 | 0.578825127 | 5.928122471 | 0.65772148 |
| hsa-miR-3161_st | 8.078330917 | 0.393578397 | 8.028612812 | 0.70304002 |
| hsa-miR-3162_st | 13.61129537 | 0.382647617 | 13.74016591 | 0.281567056 |
| hsa-miR-1234_st | 7.430496844 | 0.24346812 | 7.804084768 | 0.341248964 |
| hsa-miR-3164_st | 8.280812703 | 0.865735498 | 7.767042678 | 0.456826565 |
| hsa-miR-516b-star_st | 3.687851166 | 0.236473182 | 3.940822171 | 0.543666218 |
| hsa-miR-3166_st | 4.678145587 | 0.541500516 | 4.805545909 | 0.446833234 |
| hsa-miR-4327_st | 12.74712647 | 0.402828131 | 12.92894737 | 0.355793001 |
| hsa-miR-3168_st | 3.140494166 | 0.36004325 | 3.27220798 | 0.534956773 |
| hsa-miR-3169_st | 4.75153724 | 0.309753014 | 4.616880149 | 0.571050774 |
| hsa-miR-3170_st | 7.187293277 | 0.589324576 | 6.67695758 | 0.597061822 |
| hsa-miR-3171_st | 3.231063903 | 0.475030581 | 3.637849204 | 0.347724364 |
| hsa-miR-3172_st | 14.58285831 | 0.734537339 | 15.41871443 | 0.763529026 |
| hsa-miR-374b-star_st | 3.605525421 | 0.272833897 | 3.818550782 | 0.193224732 |
| hsa-miR-34c-5p_st | 3.945093199 | 0.261896137 | 4.136344236 | 0.309643599 |
| hsa-miR-758_st | 3.234208461 | 0.329757099 | 3.792955448 | 0.454477117 |
| hsa-miR-3176_st | 11.51930491 | 0.445282637 | 11.65911602 | 0.438608334 |
| hsa-miR-606_st | 2.954713825 | 0.186894213 | 3.228249322 | 0.510867262 |
| hsa-miR-3178_st | 15.17192555 | 0.549934826 | 15.51887145 | 0.507361375 |
| hsa-miR-3179_st | 4.588186265 | 1.035317464 | 4.467336367 | 0.693758702 |
| hsa-miR-3180-3p_st | 14.44952559 | 0.392112236 | 14.64098154 | 0.45564938 |
| hsa-miR-597_st | 3.149339096 | 0.221305103 | 3.359079574 | 0.299809355 |
| hsa-miR-148b-star_st | 3.157023161 | 0.103831491 | 3.29534718 | 0.394187076 |
| hsa-miR-3182_st | 4.213787575 | 0.639297041 | 4.131789963 | 0.391402982 |
| hsa-miR-3183_st | 6.227052298 | 0.499115078 | 6.114589632 | 0.701330752 |
| hsa-miR-144-star_st | 3.489085506 | 0.337877935 | 3.955255478 | 0.374795219 |
| hsa-miR-3185_st | 14.87958507 | 0.48906285 | 15.23627387 | 0.493389999 |
| hsa-miR-4270_st | 15.01088289 | 0.549871585 | 15.41615148 | 0.378861452 |
| hsa-miR-3186-5p_st | 5.821172624 | 0.760935079 | 5.549492043 | 0.402767751 |
| hsa-miR-3187_st | 13.29828434 | 0.406092356 | 13.41204636 | 0.457200818 |
| hsa-miR-3188_st | 13.77097499 | 0.488316812 | 14.01067639 | 0.407855668 |
| hsa-miR-3189_st | 8.05952106 | 0.689246615 | 7.673060932 | 0.504908616 |
| hsa-miR-548b-3p_st | 3.364265581 | 0.423719651 | 3.730346207 | 0.523724925 |
| hsa-miR-99a-star_st | 4.762006882 | 0.366698021 | 5.07411644 | 0.711002596 |
| hsa-miR-376a-star_st | 2.508795248 | 0.413876151 | 3.141246692 | 0.522671865 |
| hsa-miR-216b_st | 3.593037614 | 0.443346073 | 3.731855827 | 0.317200087 |
| hsa-miR-3193_st | 7.021865923 | 0.471895494 | 7.103500797 | 0.370434753 |
| hsa-miR-548v_st | 2.845068859 | 0.205041887 | 3.396418556 | 0.508541998 |
| hsa-miR-26a-1-star_st | 3.573250018 | 0.574812573 | 3.691151571 | 0.364309414 |
| hsa-miR-1291_st | 8.889656447 | 0.402786491 | 9.530605779 | 0.4661868 |
| hsa-miR-3197_st | 15.08746937 | 0.490299136 | 15.3427786 | 0.481987336 |
| hsa-miR-629_st | 7.705268389 | 0.300513199 | 7.533290481 | 0.590257946 |
| hsa-miR-3199_st | 5.728620002 | 0.515684189 | 5.530124897 | 0.530147228 |
| hsa-miR-190b_st | 2.609491809 | 0.249716498 | 3.237094422 | 0.355862875 |
| hsa-miR-32_st | 2.765905493 | 0.725766961 | 3.332213769 | 0.474489246 |
| hsa-miR-3200_st | 4.050219102 | 0.67131976 | 4.294935738 | 0.502940386 |
| hsa-miR-3201_st | 4.231262337 | 0.564257918 | 4.164140932 | 0.444900893 |
| hsa-miR-3202_st | 8.352925569 | 0.584868291 | 7.687301107 | 0.890924001 |
| hsa-miR-320a_st | 12.08249638 | 0.448833517 | 12.22087357 | 0.328277944 |
| hsa-miR-4289_st | 6.454167493 | 0.331477325 | 6.602280966 | 0.764782683 |
| hsa-miR-655_st | 2.684284953 | 0.424472471 | 3.089853494 | 0.631801198 |
| hsa-miR-320b_st | 10.9903144 | 0.204944235 | 11.19330749 | 0.376207817 |
| hsa-miR-219-5p_st | 2.742751789 | 0.415606475 | 3.340970919 | 0.685333818 |
| hsa-miR-323-3p_st | 4.496084213 | 0.275252466 | 4.406669614 | 0.555964999 |
| hsa-miR-323-5p_st | 11.08988762 | 0.561962492 | 11.10042896 | 0.763650438 |
| hsa-miR-323b-3p_st | 4.053537932 | 0.325737681 | 3.878949006 | 0.275697756 |
| hsa-miR-103-as_st | 3.699003147 | 0.234688546 | 4.115786955 | 0.301352484 |
| hsa-miR-424-star_st | 8.403914726 | 0.285606249 | 8.718096087 | 0.412283857 |
| hsa-miR-33a-star_st | 3.854974817 | 0.511255348 | 4.024523327 | 0.574469735 |
| hsa-miR-325_st | 5.096289688 | 0.539990458 | 4.923856584 | 0.648216164 |
| hsa-miR-590-3p_st | 2.908542911 | 0.520373044 | 3.378046505 | 0.519922219 |
| hsa-miR-328_st | 7.180185463 | 1.013616002 | 7.877897664 | 0.406027747 |
| hsa-miR-329_st | 4.138801731 | 0.716059657 | 3.708660036 | 0.353852712 |
| hsa-miR-32-star_st | 5.22691562 | 0.50880596 | 4.873028306 | 0.496570821 |
| hsa-miR-330-3p_st | 6.980399875 | 0.468357306 | 6.496837719 | 0.441196587 |
| hsa-miR-330-5p_st | 6.292974199 | 0.499851427 | 6.061895164 | 0.61460993 |
| hsa-miR-146a-star_st | 2.816475154 | 0.431718623 | 3.265816697 | 0.487030193 |
| hsa-miR-331-5p_st | 7.250211775 | 0.341458026 | 7.23226449 | 0.623240916 |
| hsa-miR-3167_st | 3.166391574 | 0.439408415 | 3.431822699 | 0.403014968 |
| hsa-miR-335-star_st | 4.317069956 | 0.684479857 | 4.211945196 | 0.793779177 |
| hsa-miR-337-3p_st | 4.978977748 | 0.976514438 | 4.421475435 | 0.672586477 |
| hsa-miR-337-5p_st | 4.096343579 | 0.472160758 | 3.723175748 | 0.301297161 |
| hsa-miR-421_st | 7.080391892 | 0.247684602 | 7.001604689 | 0.826111817 |
| hsa-miR-338-5p_st | 5.675320906 | 0.469608971 | 5.495045334 | 0.443624659 |
| hsa-miR-339-3p_st | 8.793336444 | 0.617385745 | 9.042854645 | 0.550001936 |
| hsa-miR-548a-5p_st | 4.067775729 | 0.289196833 | 4.565884157 | 0.359161266 |
| hsa-miR-33a_st | 2.76307636 | 0.655458502 | 3.185567803 | 0.494838441 |
| hsa-miR-590-5p_st | 3.312423448 | 0.470595048 | 3.621515964 | 0.431059799 |
| hsa-miR-33b_st | 3.108084971 | 0.725169784 | 3.473219948 | 0.335862482 |
| hsa-miR-616-star_st | 3.606718749 | 0.274095786 | 4.165351778 | 0.651495724 |
| hsa-miR-569_st | 3.526240331 | 0.459295113 | 3.895423302 | 0.322417623 |
| hsa-miR-340-star_st | 2.98694447 | 0.554046449 | 3.285513465 | 0.458334824 |
| hsa-miR-342-3p_st | 11.3732498 | 1.157887161 | 10.39046304 | 1.180714755 |
| hsa-miR-135b-star_st | 8.205480844 | 0.391984069 | 9.048699314 | 0.80852354 |
| hsa-miR-345_st | 9.585480521 | 1.594889684 | 10.85019504 | 1.133284879 |
| hsa-miR-1288_st | 5.364443903 | 0.698325402 | 5.330922015 | 0.329331984 |
| hsa-miR-135a_st | 2.696558326 | 0.221709873 | 3.425990512 | 0.401453854 |
| hsa-miR-34a-star_st | 5.939815746 | 1.211467671 | 5.839418084 | 1.183024953 |
| hsa-miR-34b_st | 5.714549118 | 0.606605663 | 5.12154628 | 0.491265461 |
| hsa-miR-4288_st | 4.272715494 | 0.331460388 | 4.744027818 | 0.484048948 |
| hsa-miR-222-star_st | 7.010154205 | 0.423408346 | 6.06600137 | 1.065825549 |
| hsa-miR-933_st | 10.9807005 | 0.250931797 | 11.21594032 | 0.567461039 |
| hsa-miR-361-3p_st | 6.456586866 | 1.162052241 | 6.434510586 | 0.901194256 |
| hsa-miR-224-star_st | 5.662827689 | 0.382235707 | 5.715729444 | 0.614769307 |
| hsa-miR-362-3p_st | 5.078554082 | 0.884344704 | 4.777126243 | 0.59707651 |
| hsa-miR-362-5p_st | 6.883450714 | 0.610850787 | 6.775630923 | 0.773578669 |
| hsa-miR-363_st | 6.482254601 | 1.017180257 | 5.603879304 | 0.391840835 |
| hsa-miR-363-star_st | 10.08243004 | 0.498036011 | 9.574926234 | 0.502789225 |
| hsa-miR-1979_st | 14.64560218 | 0.536484465 | 15.49046718 | 0.418231714 |
| hsa-miR-365-star_st | 12.13543589 | 0.372526476 | 12.20874954 | 0.440561784 |
| hsa-miR-548x_st | 2.677428897 | 0.467398017 | 3.201151821 | 0.509250285 |
| hsa-miR-367-star_st | 3.166847803 | 0.47317429 | 3.14813309 | 0.452140821 |
| hsa-miR-369-3p_st | 2.986712428 | 0.496993332 | 3.166547872 | 0.494886052 |
| hsa-miR-369-5p_st | 3.867273705 | 0.336511795 | 3.884941004 | 0.37813369 |
| hsa-miR-548l_st | 5.464725767 | 0.585535491 | 5.372004472 | 0.371108133 |
| hsa-miR-371-3p_st | 4.274001054 | 0.542464216 | 3.92885324 | 0.369251829 |
| hsa-miR-371-5p_st | 12.8344501 | 0.662910031 | 12.95215371 | 0.372916854 |
| hsa-miR-372_st | 4.901782833 | 0.797445159 | 4.025947333 | 0.300770079 |
| hsa-miR-373_st | 5.517532556 | 0.387278109 | 5.290146355 | 0.623551837 |
| hsa-miR-373-star_st | 12.14504458 | 0.617728942 | 12.15353873 | 0.476208252 |
| hsa-miR-374a_st | 4.751465217 | 0.864374745 | 5.118922619 | 1.441028543 |
| hsa-miR-374a-star_st | 3.270840348 | 0.601541701 | 3.553759321 | 0.448996791 |
| hsa-miR-500_st | 6.175925689 | 0.319056244 | 6.557940805 | 0.816131249 |
| hsa-miR-320c_st | 10.38973963 | 0.407609195 | 10.67449306 | 0.388955681 |
| hsa-miR-875-3p_st | 4.636702262 | 0.435967865 | 4.828379715 | 0.31268407 |
| hsa-miR-376a_st | 4.933677887 | 0.669683185 | 4.294236037 | 0.610248294 |
| hsa-miR-29b-1-star_st | 6.808470164 | 0.317346313 | 6.096397657 | 0.920674858 |
| hsa-miR-376b_st | 3.53147834 | 0.349850591 | 3.483365476 | 0.467879354 |
| hsa-miR-376c_st | 5.2147317 | 1.628087929 | 4.425695976 | 0.551211008 |
| hsa-miR-377_st | 3.667466583 | 0.415227156 | 3.543131613 | 0.493522555 |
| hsa-miR-377-star_st | 5.07903151 | 0.735981217 | 4.987155571 | 0.76282856 |
| hsa-miR-378_st | 9.155255817 | 0.243916474 | 9.178607837 | 0.505865573 |
| hsa-miR-378b_st | 9.068725602 | 0.429584292 | 9.073970889 | 0.337357753 |
| hsa-miR-378c_st | 9.59748568 | 0.27894387 | 9.26254085 | 0.298557243 |
| hsa-miR-378-star_st | 5.35873851 | 0.602845881 | 5.265886412 | 0.449391717 |
| hsa-miR-662_st | 7.076468751 | 0.251589484 | 7.521307862 | 0.643808445 |
| hsa-miR-379-star_st | 3.439635437 | 0.515726961 | 3.605072764 | 0.46302619 |
| hsa-miR-380_st | 3.263296242 | 0.432914622 | 3.492819867 | 0.446812646 |
| hsa-miR-380-star_st | 4.082739738 | 0.425954689 | 3.581011012 | 0.332861275 |
| hsa-miR-1274b_st | 14.21859216 | 0.341467194 | 14.75215676 | 0.552398068 |
| hsa-miR-382_st | 6.844108166 | 0.488625795 | 6.70775011 | 0.673161113 |
| hsa-miR-383_st | 6.630387098 | 0.756701143 | 6.138350483 | 0.413981974 |
| hsa-miR-384_st | 2.62475599 | 0.594289301 | 2.97861799 | 0.581679666 |
| hsa-miR-126-star_st | 3.090618572 | 0.412755455 | 3.709486666 | 0.596771335 |
| hsa-miR-409-5p_st | 4.447520965 | 0.203889075 | 4.42663381 | 0.305651106 |
| hsa-miR-410_st | 4.445331541 | 0.201573144 | 4.354181733 | 0.269792822 |
| hsa-miR-411_st | 4.857105619 | 0.701521849 | 4.427464496 | 0.245855902 |
| hsa-miR-411-star_st | 4.816343005 | 0.481288843 | 4.497132142 | 0.512731249 |
| hsa-miR-412_st | 5.212752521 | 0.633443336 | 4.990380937 | 0.671858367 |
| hsa-miR-3143_st | 2.934661021 | 0.510147005 | 3.376775359 | 0.57561388 |
| hsa-miR-422a_st | 9.971119243 | 0.146842825 | 9.99541583 | 0.509299395 |
| hsa-miR-628-5p_st | 3.341885906 | 0.384759617 | 3.907357379 | 0.450726527 |
| hsa-miR-423-5p_st | 13.50054002 | 0.277482869 | 13.52187494 | 0.413675832 |
| hsa-miR-27b-star_st | 6.656510445 | 0.431747539 | 6.260466471 | 0.792349775 |
| hsa-miR-499-5p_st | 3.494440974 | 0.256101375 | 4.108858518 | 0.597579808 |
| hsa-miR-425_st | 8.149344131 | 1.320756171 | 8.379308962 | 0.938481978 |
| hsa-miR-193b_st | 8.060880165 | 0.705997143 | 8.05284561 | 1.052165679 |
| hsa-miR-4252_st | 5.623063449 | 0.498919065 | 5.487794629 | 0.478722537 |
| hsa-miR-4253_st | 12.29975327 | 0.372284206 | 12.35023599 | 0.390757523 |
| hsa-miR-4254_st | 5.836225877 | 0.530458734 | 5.53422782 | 0.584675308 |
| hsa-miR-324-3p_st | 7.930780917 | 0.340065786 | 8.603429333 | 0.628003845 |
| hsa-miR-4329_st | 5.008222334 | 0.367787358 | 5.316564196 | 0.419411743 |
| hsa-miR-1251_st | 4.485914886 | 0.415648951 | 4.809983142 | 0.542896674 |
| hsa-miR-4258_st | 6.333090277 | 0.816073816 | 6.053779074 | 0.884127425 |
| hsa-miR-1274a_st | 10.97591948 | 0.625871027 | 11.49387492 | 0.90734772 |
| hsa-miR-425-star_st | 8.476251492 | 0.477262594 | 8.146923251 | 0.447607298 |
| hsa-miR-4260_st | 11.29415184 | 0.652082284 | 11.32935691 | 0.450697853 |
| hsa-miR-4261_st | 9.459280319 | 0.32938108 | 9.347662405 | 0.436745988 |
| hsa-miR-4262_st | 3.466167525 | 0.606225256 | 3.503654702 | 0.520792155 |
| hsa-miR-4263_st | 3.799849718 | 0.19914035 | 3.556987922 | 0.498312439 |
| hsa-miR-4264_st | 3.237469816 | 0.425485262 | 3.300635174 | 0.470200734 |
| hsa-miR-1280_st | 14.21651563 | 0.690007938 | 15.20657291 | 0.45181172 |
| hsa-miR-132-star_st | 4.476731905 | 0.643431032 | 4.262178837 | 0.536337516 |
| hsa-miR-4267_st | 6.665799105 | 0.796017696 | 6.823276764 | 0.579931915 |
| hsa-miR-4268_st | 3.54207029 | 0.517899414 | 3.838903571 | 0.400783289 |
| hsa-miR-133a_st | 5.182983801 | 0.551544575 | 5.621377515 | 0.872669765 |
| hsa-miR-888-star_st | 3.612483776 | 0.850815377 | 3.719893202 | 0.299255072 |
| hsa-miR-1260b_st | 14.32586379 | 0.576309397 | 15.36453726 | 0.425912754 |
| hsa-miR-4272_st | 3.202665144 | 0.551825936 | 3.531059082 | 0.486605851 |
| hsa-miR-4273_st | 3.58266892 | 0.353144374 | 3.550680173 | 0.480943708 |
| hsa-miR-4274_st | 5.71460909 | 0.41323424 | 5.506414306 | 0.368001989 |
| hsa-miR-4275_st | 2.624976968 | 0.544435123 | 3.228973772 | 0.564291746 |
| hsa-miR-4276_st | 2.964511238 | 0.316609281 | 2.968249899 | 0.58351474 |
| hsa-miR-4277_st | 4.014194722 | 0.317811232 | 3.900972458 | 0.373218283 |
| hsa-miR-4278_st | 7.355075966 | 0.346658372 | 7.416346046 | 0.846871807 |
| hsa-miR-4279_st | 5.36463542 | 0.696142052 | 5.024418515 | 0.665814984 |
| hsa-miR-4280_st | 5.347313844 | 0.842412079 | 4.72171649 | 0.508782947 |
| hsa-miR-4281_st | 15.26645656 | 0.57839292 | 15.93454499 | 0.436729583 |
| hsa-miR-4282_st | 2.663950547 | 0.352345956 | 2.899083144 | 0.36583047 |
| hsa-miR-4283_st | 8.849144938 | 0.439411614 | 8.751144402 | 0.647567451 |
| hsa-miR-4284_st | 15.03758748 | 0.628266437 | 15.63455107 | 0.748864147 |
| hsa-miR-23b-star_st | 5.655365132 | 0.660897667 | 6.187247095 | 0.477791019 |
| hsa-miR-574-3p_st | 8.843824407 | 0.682077023 | 9.476594798 | 0.846282809 |
| hsa-miR-4287_st | 6.31590232 | 1.140026207 | 5.760932161 | 0.27283173 |
| hsa-miR-320e_st | 9.303242502 | 0.698514747 | 9.629066345 | 0.451491017 |
| hsa-miR-339-5p_st | 7.826006619 | 0.794859971 | 8.149693895 | 1.492391906 |
| hsa-miR-505_st | 5.613178946 | 0.858046346 | 6.152618643 | 0.741390891 |
| hsa-miR-4290_st | 6.531030796 | 0.626867363 | 5.932265364 | 0.650016998 |
| hsa-miR-200b-star_st | 6.509608956 | 0.633956213 | 6.869262003 | 0.42487672 |
| hsa-miR-4292_st | 8.636472189 | 0.469237906 | 8.391395154 | 0.535534681 |
| hsa-miR-4293_st | 5.650185679 | 0.620997527 | 5.229993773 | 0.486906139 |
| hsa-miR-1246_st | 12.63293369 | 0.75307796 | 13.44082138 | 0.384326801 |
| hsa-miR-4295_st | 3.715080304 | 0.525230144 | 3.665499751 | 0.428320447 |
| hsa-miR-4296_st | 7.544380309 | 0.522269686 | 7.1948071 | 0.654218085 |
| hsa-miR-4297_st | 6.162451907 | 1.295423249 | 5.762237388 | 0.784792458 |
| hsa-miR-4298_st | 13.0932607 | 0.350959943 | 13.25279635 | 0.29991741 |
| hsa-miR-218_st | 5.845160525 | 0.85479425 | 5.29779846 | 0.949266369 |
| hsa-miR-4286_st | 13.75185093 | 0.503259743 | 14.6658497 | 0.629276818 |
| hsa-miR-4301_st | 2.879288298 | 0.537823003 | 3.268037048 | 0.614296646 |
| hsa-miR-4302_st | 6.201551747 | 0.764939016 | 6.219510564 | 0.602679532 |
| hsa-miR-365_st | 4.105421402 | 0.450902318 | 5.028442045 | 1.063069828 |
| hsa-miR-4304_st | 7.263505113 | 0.791932081 | 6.698126224 | 0.673484963 |
| hsa-miR-4305_st | 3.821391963 | 0.290637775 | 3.979572912 | 0.769965645 |
| hsa-miR-4306_st | 9.680513718 | 0.297463542 | 9.416998374 | 0.537538758 |
| hsa-miR-26a_st | 14.06265926 | 0.643366569 | 14.51988806 | 0.645907622 |
| hsa-miR-4308_st | 3.399246698 | 0.419121852 | 3.533942319 | 0.569774082 |
| hsa-miR-4309_st | 4.284476362 | 0.16993954 | 4.373928527 | 0.358853407 |
| hsa-miR-186_st | 5.964983027 | 0.667842891 | 6.197142666 | 1.160061993 |
| hsa-miR-4310_st | 5.877304755 | 0.49986974 | 5.351169653 | 0.949576648 |
| hsa-miR-4311_st | 6.00514139 | 0.554220506 | 5.507993494 | 0.424596731 |
| hsa-miR-4312_st | 6.700974102 | 0.750195332 | 6.499226613 | 0.715576367 |
| hsa-miR-4313_st | 5.673163784 | 0.498057922 | 6.071167779 | 0.740165349 |
| hsa-miR-4314_st | 9.92478384 | 0.776279846 | 9.355904742 | 0.501936432 |
| hsa-miR-4315_st | 5.040244261 | 0.662159701 | 4.39870044 | 0.521955231 |
| hsa-miR-149_st | 6.25624792 | 0.788758754 | 6.594642354 | 0.674518316 |
| hsa-miR-106b-star_st | 7.091804467 | 0.703222616 | 7.23142957 | 0.518682637 |
| hsa-miR-4318_st | 5.164813579 | 0.431502162 | 4.91263378 | 0.231431961 |
| hsa-miR-4319_st | 5.240884066 | 0.474531261 | 4.851279225 | 0.976481454 |
| hsa-miR-29b-2-star_st | 7.198521986 | 0.498788185 | 7.432710584 | 0.498655518 |
| hsa-miR-432_st | 7.644471654 | 0.687486285 | 7.77954842 | 0.428486803 |
| hsa-miR-4320_st | 4.157803974 | 0.543855896 | 4.106276132 | 0.398947015 |
| hsa-miR-522_st | 3.752102979 | 0.369402226 | 4.152979731 | 0.429654915 |
| hsa-miR-4322_st | 13.18611103 | 0.416549346 | 13.29699992 | 0.417183912 |
| hsa-miR-4323_st | 5.546476182 | 0.613154407 | 4.929921719 | 0.449407249 |
| hsa-miR-93-star_st | 4.648957304 | 0.398472432 | 5.341887775 | 0.663163305 |
| hsa-miR-4325_st | 3.472812966 | 0.766111651 | 3.399023333 | 0.501138761 |
| hsa-miR-4326_st | 5.014180064 | 0.318783572 | 4.940786774 | 0.455168366 |
| hsa-miR-874_st | 13.10588124 | 0.455066955 | 13.29930115 | 0.440358508 |
| hsa-miR-4328_st | 3.446019697 | 0.553353619 | 3.282089766 | 0.502911911 |
| hsa-let-7e-star_st | 4.186213708 | 0.444279002 | 4.438840941 | 0.583980659 |
| hsa-miR-720_st | 15.16426802 | 0.588035966 | 15.92381617 | 0.488269767 |
| hsa-miR-433_st | 6.138137944 | 0.757675594 | 5.794120385 | 0.610340815 |
| hsa-miR-4330_st | 6.186930612 | 1.016268509 | 5.567521249 | 1.12727563 |
| hsa-miR-448_st | 3.63939461 | 1.007526253 | 4.052723609 | 0.422843127 |
| hsa-miR-449a_st | 4.057089965 | 0.373201367 | 3.807615469 | 0.451646959 |
| hsa-miR-449b_st | 5.593402581 | 0.571492419 | 5.24026024 | 0.633033943 |
| hsa-miR-1975_st | 15.17808808 | 0.544027633 | 15.91178915 | 0.500764593 |
| hsa-miR-449c_st | 7.356361981 | 0.810774526 | 7.251217892 | 0.973970395 |
| hsa-miR-449c-star_st | 4.818189122 | 0.514254502 | 4.631370238 | 0.391830188 |
| hsa-miR-1_st | 4.389598188 | 0.654375264 | 4.779897548 | 1.035204203 |
| hsa-miR-450b-3p_st | 2.69492697 | 0.419831728 | 3.024592864 | 0.712510111 |
| hsa-miR-450b-5p_st | 2.804658413 | 0.547349813 | 3.133244669 | 0.574826 |
| hsa-miR-451_st | 13.21390629 | 1.084231782 | 13.1075826 | 0.817803403 |
| hsa-miR-452_st | 7.377192127 | 0.625990407 | 6.838818678 | 0.440390878 |
| hsa-miR-452-star_st | 3.605993958 | 0.296250574 | 3.858275996 | 0.391258058 |
| hsa-miR-548b-5p_st | 4.351876204 | 0.244819683 | 4.352305991 | 0.305134798 |
| hsa-miR-454-star_st | 4.234173336 | 0.323346874 | 3.865179456 | 0.479665683 |
| hsa-miR-455-3p_st | 7.863868663 | 0.983258095 | 8.029366775 | 0.798250584 |
| hsa-miR-455-5p_st | 4.221440418 | 0.696422608 | 4.316368924 | 0.935095353 |
| hsa-miR-466_st | 5.76689287 | 0.167050777 | 5.622942711 | 1.064755284 |
| hsa-miR-483-3p_st | 6.320377239 | 0.637331415 | 6.460073402 | 0.730241126 |
| hsa-miR-17_st | 9.911407432 | 0.819706418 | 9.851855822 | 1.085095739 |
| hsa-miR-320d_st | 9.309138874 | 0.75865606 | 9.861194361 | 0.646697412 |
| hsa-miR-485-3p_st | 6.113855202 | 0.935453875 | 5.63373336 | 0.654373784 |
| hsa-miR-485-5p_st | 7.570602764 | 0.66882958 | 7.27549331 | 0.61145025 |
| hsa-miR-486-3p_st | 10.64593355 | 0.403981283 | 10.444397 | 0.696916964 |
| hsa-miR-486-5p_st | 7.958980322 | 0.994160211 | 8.197724973 | 0.68125073 |
| hsa-miR-644_st | 3.622779929 | 0.627546373 | 3.633581198 | 0.478493508 |
| hsa-miR-3152_st | 8.056812298 | 1.047881398 | 7.709297978 | 0.631389848 |
| hsa-miR-488_st | 3.781760961 | 0.385671248 | 3.69207105 | 0.482256574 |
| hsa-miR-488-star_st | 3.809190571 | 0.468919659 | 3.58041264 | 0.510211437 |
| hsa-miR-450a_st | 4.130970306 | 0.375060776 | 4.367793549 | 0.702786113 |
| hsa-miR-20a_st | 9.608873978 | 0.985258266 | 9.154240888 | 1.570959528 |
| hsa-miR-484_st | 7.397842875 | 0.867635174 | 8.167388556 | 0.811779194 |
| hsa-miR-491-3p_st | 4.855686496 | 0.242751543 | 4.84565073 | 0.414393313 |
| hsa-miR-491-5p_st | 9.424070653 | 0.422905689 | 9.282838637 | 0.541984612 |
| hsa-miR-197_st | 6.810682237 | 0.451539328 | 7.458081014 | 0.672627556 |
| hsa-miR-493_st | 5.809860932 | 0.652961754 | 5.721542924 | 0.501631458 |
| hsa-miR-493-star_st | 4.882266166 | 0.792426882 | 4.326393478 | 0.393539118 |
| hsa-miR-494_st | 15.19879237 | 0.537914517 | 15.71807116 | 0.302046259 |
| hsa-miR-495_st | 4.126178727 | 0.824723165 | 3.764773266 | 0.550530107 |
| hsa-miR-496_st | 3.013409544 | 0.537434768 | 3.24066256 | 0.340835478 |
| hsa-miR-497_st | 8.617519141 | 1.187029325 | 9.31253402 | 1.274820565 |
| hsa-miR-497-star_st | 3.617157328 | 0.411431969 | 3.541402348 | 0.288230566 |
| hsa-miR-498_st | 12.18788868 | 0.490031654 | 12.26996738 | 0.464060825 |
| hsa-miR-499-3p_st | 3.935294503 | 0.273929569 | 4.122394462 | 0.377211944 |
| hsa-miR-24-1-star_st | 3.99375153 | 0.649529313 | 4.371628213 | 1.095631286 |
| hsa-miR-340_st | 4.975184349 | 0.670618291 | 5.315422115 | 1.171203165 |
| hsa-miR-500b_st | 5.032541521 | 0.708096953 | 4.874411859 | 0.40238791 |
| hsa-miR-500-star_st | 9.469760732 | 0.243233572 | 9.55955348 | 0.377213895 |
| hsa-miR-501-3p_st | 9.481838831 | 0.440340115 | 9.546448548 | 0.454813052 |
| hsa-miR-501-5p_st | 6.318312489 | 0.092184448 | 6.420445453 | 0.618899598 |
| hsa-miR-502-3p_st | 9.21758196 | 0.185821322 | 9.335647904 | 0.323462784 |
| hsa-miR-502-5p_st | 5.671931897 | 0.529603559 | 5.403143638 | 0.5101396 |
| hsa-miR-503_st | 10.44903327 | 0.252694404 | 10.65776662 | 0.603084434 |
| hsa-miR-192_st | 7.289860068 | 0.925112021 | 7.360684321 | 1.337981563 |
| hsa-let-7g_st | 12.90594635 | 0.949684998 | 13.55886367 | 0.693278644 |
| hsa-miR-505-star_st | 7.694368548 | 0.362862247 | 7.629871326 | 0.595733983 |
| hsa-miR-506_st | 3.603103318 | 0.389611026 | 3.950790466 | 0.37721683 |
| hsa-miR-507_st | 3.400436032 | 0.345495971 | 3.384626045 | 0.54930887 |
| hsa-miR-508-3p_st | 3.175469508 | 0.373948827 | 3.412628275 | 0.613238957 |
| hsa-miR-508-5p_st | 9.332315514 | 0.481013307 | 9.176048356 | 0.479135237 |
| hsa-miR-509-3-5p_st | 7.741232586 | 0.835204717 | 6.965159833 | 1.084750535 |
| hsa-miR-509-3p_st | 6.163920072 | 0.678640656 | 6.203675997 | 0.598356854 |
| hsa-miR-106a_st | 9.68177744 | 0.931288539 | 9.644741005 | 1.143503481 |
| hsa-miR-106b_st | 8.745261083 | 0.744895877 | 9.031126071 | 1.148491266 |
| hsa-miR-511_st | 3.369775383 | 0.473241432 | 3.193442924 | 0.54137822 |
| hsa-miR-512-3p_st | 5.469054611 | 0.418419699 | 5.239480308 | 0.469294305 |
| hsa-miR-512-5p_st | 6.40508112 | 0.85242686 | 5.778311262 | 0.385036549 |
| hsa-miR-513a-3p_st | 2.938403512 | 0.707064858 | 3.108604996 | 0.324891244 |
| hsa-miR-23a_st | 13.40521412 | 0.83347076 | 13.99358348 | 0.686196145 |
| hsa-miR-25_st | 9.97963034 | 0.989496369 | 10.39025555 | 0.95623205 |
| hsa-miR-598_st | 5.757616441 | 0.675506113 | 6.053068424 | 1.053244455 |
| hsa-miR-1260_st | 10.6909495 | 0.949850747 | 11.79939741 | 0.512065319 |
| hsa-miR-514b-3p_st | 3.703986333 | 0.614638402 | 3.627111192 | 0.618112801 |
| hsa-miR-514b-5p_st | 11.29318544 | 0.384550635 | 11.31621348 | 0.457639244 |
| hsa-miR-515-3p_st | 4.421534265 | 0.559473177 | 4.47155921 | 0.45152108 |
| hsa-miR-515-5p_st | 3.421915412 | 0.46684992 | 3.637529256 | 0.417765063 |
| hsa-miR-516a-3p_st | 3.545396323 | 0.53388883 | 3.963442221 | 0.45977701 |
| hsa-miR-27a_st | 11.89840789 | 1.022894109 | 12.23697775 | 0.984413648 |
| hsa-miR-100_st | 11.53783501 | 0.875241413 | 12.32670387 | 0.953919607 |
| hsa-miR-101_st | 5.706489929 | 1.191460646 | 5.659717607 | 1.349211302 |
| hsa-miR-517a_st | 3.271203117 | 0.665778267 | 3.074939274 | 0.603651488 |
| hsa-miR-517b_st | 3.660535626 | 0.402079349 | 3.767684645 | 0.395297551 |
| hsa-miR-517c_st | 2.625496505 | 0.495948538 | 2.96173305 | 0.510418433 |
| hsa-miR-517-star_st | 5.616741984 | 0.383400145 | 5.356683141 | 0.438582595 |
| hsa-miR-518a-3p_st | 3.774515979 | 0.363039245 | 3.73146198 | 0.607125376 |
| hsa-miR-518a-5p_st | 7.546676851 | 0.895782651 | 7.51740602 | 0.772240288 |
| hsa-miR-518b_st | 3.957233855 | 0.431594022 | 4.028948225 | 0.69391213 |
| hsa-miR-518c_st | 2.820454585 | 0.516204781 | 3.252104081 | 0.538896342 |
| hsa-miR-301a_st | 4.001778778 | 0.790727646 | 4.377887613 | 0.789737028 |
| hsa-miR-518d-3p_st | 4.029427522 | 0.293696172 | 4.160632542 | 0.312335768 |
| hsa-miR-518d-5p_st | 6.959263894 | 0.529187537 | 6.837507757 | 0.444413667 |
| hsa-miR-518e_st | 2.942994704 | 0.702278282 | 3.099102503 | 0.301362312 |
| hsa-let-7f_st | 13.53844043 | 0.957531718 | 14.53149322 | 0.796521392 |
| hsa-miR-518f_st | 3.183794886 | 0.458314101 | 3.240782689 | 0.565568002 |
| hsa-miR-1826_st | 15.28967372 | 0.572637283 | 16.03679633 | 0.513337747 |
| hsa-miR-519a_st | 2.883068029 | 0.607674584 | 3.201570696 | 0.566257143 |
| hsa-miR-519a-star_st | 6.385948319 | 0.739472779 | 6.178999125 | 0.912215206 |
| hsa-miR-519b-3p_st | 2.966350161 | 0.530305981 | 3.394314329 | 0.603805215 |
| hsa-miR-1283_st | 3.818149664 | 0.362926775 | 4.008192314 | 0.396653707 |
| hsa-miR-519c-3p_st | 2.537597609 | 0.430574995 | 2.88125684 | 0.737785061 |
| hsa-miR-519c-5p_st | 6.773995045 | 0.419518244 | 6.311720462 | 0.639096658 |
| hsa-miR-519d_st | 3.719747083 | 0.486899762 | 3.729126735 | 0.282970933 |
| hsa-miR-519e_st | 3.666986117 | 0.537493088 | 4.05185554 | 0.380468692 |
| hsa-miR-519e-star_st | 7.852978474 | 0.806345684 | 7.463457108 | 0.47120124 |
| hsa-miR-520a-3p_st | 4.076495011 | 0.390757527 | 3.668711681 | 0.430966895 |
| hsa-miR-1908_st | 15.2900801 | 0.572704869 | 16.00657699 | 0.46917991 |
| hsa-miR-520b_st | 3.376673639 | 0.624027974 | 3.437756838 | 0.281553526 |
| hsa-miR-520c-3p_st | 4.124562316 | 0.670591664 | 4.20618397 | 0.519514433 |
| hsa-miR-520c-5p_st | 7.225873823 | 0.648600383 | 7.010128898 | 0.658126205 |
| hsa-miR-181a-star_st | 5.36162966 | 0.876368995 | 6.105419351 | 1.335539287 |
| hsa-miR-21_st | 12.27247487 | 0.867928278 | 11.72436469 | 1.579673894 |
| hsa-miR-520e_st | 3.34690233 | 0.461189419 | 3.332044154 | 0.580799756 |
| hsa-miR-520f_st | 3.569338857 | 0.494416808 | 3.675205079 | 0.569323742 |
| hsa-miR-520g_st | 3.715628237 | 0.263020239 | 3.790170258 | 0.426921536 |
| hsa-miR-520h_st | 3.581612961 | 0.629410226 | 3.615219658 | 0.301455663 |
| hsa-miR-521_st | 3.447586947 | 0.441975075 | 3.466793781 | 0.365156685 |
| hsa-miR-302a-star_st | 4.119283472 | 0.39086816 | 4.163693066 | 0.447726682 |
| hsa-miR-886-3p_st | 10.43594576 | 1.092375062 | 11.19634523 | 1.762310894 |
| hsa-miR-523_st | 3.250858763 | 0.678276609 | 3.526151865 | 0.428435737 |
| hsa-miR-29a_st | 12.88670674 | 0.881472224 | 13.3682967 | 0.902596082 |
| hsa-miR-524-3p_st | 4.280548548 | 0.231819334 | 4.288000107 | 0.36687549 |
| hsa-miR-142-3p_st | 3.39962904 | 1.044278077 | 3.278354139 | 0.381792283 |
| hsa-miR-525-3p_st | 3.284680499 | 0.529736098 | 3.556084102 | 0.473997043 |
| hsa-miR-525-5p_st | 6.339650463 | 0.388103515 | 6.633080915 | 0.666712414 |
| hsa-miR-762_st | 15.29003597 | 0.572739171 | 16.00868256 | 0.47157006 |
| hsa-miR-423-3p_st | 8.710366702 | 0.481880908 | 9.375177054 | 0.695775063 |
| hsa-miR-449b-star_st | 8.194175214 | 0.756235824 | 8.335174354 | 0.567521543 |
| hsa-miR-527_st | 7.767903981 | 0.551276159 | 7.80970047 | 0.744666167 |
| hsa-miR-532-3p_st | 7.354938434 | 0.920695458 | 8.044950525 | 0.85382626 |
| hsa-miR-532-5p_st | 7.887195197 | 0.778924247 | 8.068299035 | 0.976940353 |
| hsa-miR-539_st | 4.812742975 | 0.70860201 | 4.243994205 | 0.576836564 |
| hsa-let-7a_st | 13.49390454 | 0.924028709 | 14.52686123 | 0.602367564 |
| hsa-miR-541-star_st | 6.563153172 | 0.273867123 | 6.691637229 | 0.544815687 |
| hsa-miR-542-3p_st | 7.313408328 | 0.425071808 | 7.106489712 | 0.687807281 |
| hsa-miR-542-5p_st | 7.676313231 | 0.903749101 | 7.6481249 | 0.568575219 |
| hsa-miR-543_st | 4.628013935 | 0.368754234 | 5.007496684 | 0.468221753 |
| hsa-miR-544_st | 2.899953315 | 0.754057912 | 3.065592882 | 0.683140413 |
| hsa-miR-544b_st | 4.095844387 | 0.488886763 | 4.055365573 | 0.301503822 |
| hsa-miR-545_st | 2.963886737 | 0.517620244 | 3.251981938 | 0.520254616 |
| hsa-miR-545-star_st | 2.37005741 | 0.611119253 | 2.756596607 | 0.621606158 |
| hsa-miR-548a-3p_st | 2.610282927 | 0.82238186 | 3.042339563 | 0.605724721 |
| hsa-miR-2861_st | 15.28944857 | 0.572764211 | 16.02630754 | 0.496698468 |
| hsa-miR-31_st | 9.738405386 | 0.786490338 | 7.506889665 | 2.175620322 |
| hsa-miR-367_st | 3.04675118 | 0.510897062 | 3.453822947 | 0.635530205 |
| hsa-miR-548c-3p_st | 2.410143039 | 0.963087824 | 2.779940361 | 0.721422528 |
| hsa-miR-548c-5p_st | 5.275070459 | 1.546908633 | 4.8886108 | 0.751328454 |
| hsa-miR-548d-3p_st | 3.49829656 | 0.397604535 | 3.521580668 | 0.449347075 |
| hsa-miR-548d-5p_st | 4.42899606 | 0.912856187 | 4.487909444 | 0.671734608 |
| hsa-miR-548e_st | 3.029536788 | 0.694735974 | 3.139195386 | 0.645159795 |
| hsa-miR-548f_st | 2.890405204 | 0.630671192 | 3.402453836 | 0.56525575 |
| hsa-miR-548g_st | 2.555785224 | 0.914644246 | 3.09329926 | 0.540928993 |
| hsa-miR-548h_st | 3.715772486 | 0.398305069 | 3.607193114 | 0.376311953 |
| hsa-miR-548i_st | 4.445010549 | 0.460413642 | 4.414332592 | 0.563375846 |
| hsa-miR-548j_st | 4.816342289 | 0.480893369 | 4.398913565 | 0.358260768 |
| hsa-miR-548k_st | 3.553971503 | 0.199541141 | 3.697438969 | 0.427280207 |
| hsa-miR-526b-star_st | 3.174061704 | 0.691462473 | 3.510399179 | 0.623128916 |
| hsa-miR-548m_st | 3.913950845 | 0.502544334 | 3.922647559 | 0.49628704 |
| hsa-miR-548n_st | 3.35340513 | 0.621449025 | 3.36614022 | 0.455752058 |
| hsa-miR-548o_st | 2.729832663 | 0.534874975 | 3.01761268 | 0.609969399 |
| hsa-miR-548p_st | 3.005461492 | 0.435036534 | 3.112686142 | 0.470522725 |
| hsa-miR-548q_st | 9.784011814 | 0.652070751 | 9.777873729 | 0.686874287 |
| hsa-miR-548s_st | 3.267890179 | 0.311821367 | 3.429383231 | 0.376135277 |
| hsa-miR-548t_st | 4.254581329 | 0.552042309 | 4.100555118 | 0.361903307 |
| hsa-miR-548u_st | 2.735443914 | 0.709380949 | 2.973809617 | 0.496135649 |
| hsa-miR-638_st | 15.29028291 | 0.572807741 | 16.03093398 | 0.502496496 |
| hsa-miR-548w_st | 4.590615788 | 0.973916847 | 4.566189714 | 0.713827381 |
| hsa-miR-3119_st | 3.000516723 | 0.697574429 | 3.257485837 | 0.55539896 |
| hsa-miR-549_st | 4.060425511 | 0.522020166 | 3.983522539 | 0.447249415 |
| hsa-miR-205_st | 5.868416527 | 0.987107459 | 6.415302921 | 2.382372521 |
| hsa-miR-550-star_st | 5.120996416 | 0.244514565 | 4.806450169 | 0.523105909 |
| hsa-miR-551a_st | 4.84345183 | 0.563323547 | 4.718158107 | 0.292267281 |
| hsa-miR-93_st | 9.052819722 | 1.048751576 | 9.602934567 | 0.861987343 |
| hsa-miR-551b-star_st | 11.26848234 | 0.313339517 | 11.51505499 | 0.472085822 |
| hsa-let-7d-star_st | 7.193338978 | 0.271677733 | 8.029863583 | 0.576215859 |
| hsa-miR-553_st | 3.316698717 | 0.503749243 | 3.47194354 | 0.470110839 |
| hsa-miR-554_st | 4.232798834 | 0.61087144 | 4.169610062 | 0.414779198 |
| hsa-miR-555_st | 3.931142226 | 0.335045289 | 4.215636391 | 0.515013168 |
| hsa-miR-556-3p_st | 3.366659802 | 0.553422029 | 3.704729374 | 0.651918059 |
| hsa-miR-556-5p_st | 2.62303551 | 0.706559957 | 3.313155654 | 0.438176022 |
| hsa-miR-557_st | 12.2567656 | 0.429379501 | 12.16115603 | 0.474097753 |
| hsa-miR-558_st | 3.184928756 | 0.846770143 | 3.452420312 | 0.472527971 |
| hsa-miR-559_st | 3.262389309 | 0.644459838 | 3.480608437 | 0.50623961 |
| hsa-miR-561_st | 3.058835091 | 0.603788142 | 3.365824062 | 0.528766736 |
| hsa-miR-562_st | 2.974325726 | 0.559682007 | 3.535781894 | 0.525761053 |
| hsa-miR-563_st | 4.124724553 | 0.293130513 | 4.059812431 | 0.589158686 |
| hsa-miR-361-5p_st | 10.28905064 | 0.705634134 | 10.8588484 | 0.685039395 |
| hsa-miR-566_st | 6.679007388 | 0.358665264 | 6.57084305 | 0.762437039 |
| hsa-miR-567_st | 4.161498595 | 0.357710399 | 4.179227014 | 0.468556718 |
| hsa-miR-568_st | 2.784968005 | 0.545654278 | 3.263821719 | 0.369748616 |
| hsa-miR-29c_st | 10.98554772 | 0.838199398 | 11.70967646 | 0.908751687 |
| hsa-miR-570_st | 3.374409593 | 0.689094926 | 3.483412582 | 0.390703995 |
| hsa-miR-571_st | 4.101191188 | 0.398495693 | 4.417011732 | 0.487564824 |
| hsa-miR-572_st | 14.39070447 | 0.519961869 | 14.51032918 | 0.499463303 |
| hsa-miR-573_st | 3.33537827 | 0.473493568 | 3.580436095 | 0.475203324 |
| hsa-miR-132_st | 6.409005228 | 0.806207614 | 6.482435009 | 0.847705632 |
| hsa-miR-574-5p_st | 8.108251814 | 0.19086485 | 8.011848197 | 0.492410397 |
| hsa-let-7d_st | 13.00989227 | 0.969917961 | 4.05239565 | 0.684022426 |
| hsa-miR-576-3p_st | 4.658689063 | 0.441771213 | 4.619422191 | 0.38411683 |
| hsa-miR-576-5p_st | 4.335024532 | 0.596901908 | 4.022535217 | 0.519644766 |
| hsa-miR-577_st | 3.319513114 | 0.616278548 | 3.42955308 | 0.4765682 |
| hsa-miR-578_st | 4.32295386 | 0.450696413 | 4.463240952 | 0.520416353 |
| hsa-miR-579_st | 3.282298524 | 0.3249947 | 3.642694969 | 0.327077149 |
| hsa-miR-580_st | 2.980375615 | 0.655879331 | 3.40622339 | 0.525531015 |
| hsa-miR-191_st | 11.53521188 | 1.016026799 | 12.21787403 | 0.831991222 |
| hsa-miR-582-3p_st | 4.03649104 | 0.586109493 | 4.190053542 | 0.633941549 |
| hsa-miR-582-5p_st | 4.294780284 | 1.216725462 | 3.612554114 | 0.669510381 |
| hsa-miR-1915_st | 15.28992116 | 0.572684222 | 16.03640504 | 0.512507373 |
| hsa-let-7c_st | 12.92940572 | 0.905539378 | 13.92261478 | 0.669396526 |
| hsa-miR-585_st | 3.891254254 | 0.499421956 | 3.734859803 | 0.494313938 |
| hsa-miR-586_st | 3.137645614 | 0.502218636 | 3.323442187 | 0.58307489 |
| hsa-miR-324-5p_st | 7.901753598 | 0.535488121 | 8.422669425 | 1.238809139 |
| hsa-miR-588_st | 5.50223679 | 0.449128666 | 5.19651698 | 0.521013227 |
| hsa-miR-589_st | 5.100173667 | 0.568284802 | 4.783919018 | 0.608355677 |
| hsa-miR-589-star_st | 5.144599194 | 0.44850186 | 5.218465403 | 0.510398323 |
| hsa-miR-188-5p_st | 8.458269738 | 0.628579965 | 8.982324701 | 0.405872937 |
| hsa-miR-103_st | 11.57943947 | 1.03845505 | 12.35592736 | 0.859634731 |
| hsa-miR-591_st | 4.276292827 | 0.69395926 | 4.136078358 | 0.481789689 |
| hsa-miR-592_st | 3.277573248 | 0.387020942 | 3.279188464 | 0.37909087 |
| hsa-miR-593_st | 6.21139341 | 0.596413941 | 6.33737736 | 0.733721387 |
| hsa-miR-23b_st | 13.23856481 | 0.848654137 | 14.07858579 | 0.66207202 |
| hsa-miR-595_st | 7.257458881 | 0.514116023 | 7.292184768 | 0.414813816 |
| hsa-miR-99a_st | 11.3875049 | 1.007363751 | 12.3463053 | 0.847981797 |
| hsa-miR-15b_st | 9.442335529 | 0.87290396 | 10.0199783 | 0.84067346 |
| hsa-miR-135b_st | 3.22945361 | 0.798396855 | 4.304817665 | 1.218005328 |
| hsa-miR-599_st | 2.76524505 | 0.572996946 | 3.245363125 | 0.545230737 |
| hsa-miR-600_st | 6.423478596 | 0.694574436 | 7.278047979 | 0.984262659 |
| hsa-miR-107_st | 11.41799085 | 1.035072958 | 12.31077559 | 0.791342542 |
| hsa-miR-602_st | 13.97699159 | 0.378094594 | 14.08554857 | 0.485198154 |
| hsa-miR-603_st | 2.727733058 | 0.627721695 | 3.142942335 | 0.390579257 |
| hsa-miR-3065-5p_st | 3.808900144 | 0.442606824 | 4.418614984 | 0.809191034 |
| hsa-miR-605_st | 6.249296511 | 1.054617464 | 5.683706597 | 0.786049366 |
| hsa-miR-24_st | 12.25538638 | 0.953017587 | 12.80686121 | 0.782954798 |
| hsa-miR-607_st | 3.255728844 | 0.58959146 | 3.500577783 | 0.428756962 |
| hsa-miR-608_st | 9.590752811 | 0.590820876 | 9.53961264 | 0.58609811 |
| hsa-miR-609_st | 3.996156246 | 0.652157964 | 3.597583444 | 0.530770225 |
| hsa-miR-3196_st | 15.29002273 | 0.57271799 | 16.03737462 | 0.513859846 |
| hsa-miR-26b_st | 11.45504823 | 0.943624605 | 12.16655225 | 1.203426527 |
| hsa-miR-28-5p_st | 8.391016946 | 1.046500749 | 9.30035099 | 0.740055614 |
| hsa-miR-613_st | 4.648040923 | 0.614347931 | 4.172820101 | 0.560761512 |
| hsa-miR-338-3p_st | 3.798822829 | 0.590491859 | 4.38968126 | 0.949680643 |
| hsa-miR-615-3p_st | 7.737253651 | 0.72675537 | 7.469800936 | 0.654218859 |
| hsa-miR-454_st | 5.197679188 | 0.858166962 | 5.712982854 | 1.42285409 |
| hsa-miR-616_st | 8.072487885 | 0.683217845 | 7.689660531 | 0.469271941 |
| hsa-miR-151-5p_st | 10.80629037 | 1.103200741 | 11.64328037 | 0.729890198 |
| hsa-miR-617_st | 7.551803162 | 1.170711196 | 7.332988086 | 0.940263891 |
| hsa-miR-128_st | 7.202727337 | 1.15596349 | 7.869565664 | 0.931548446 |
| hsa-miR-619_st | 4.728674725 | 0.323584208 | 4.662569665 | 0.316095423 |
| hsa-miR-620_st | 3.802758564 | 0.358673474 | 3.963262056 | 0.668118775 |
| hsa-miR-621_st | 3.74897129 | 0.561999668 | 3.955204814 | 0.470633985 |
| hsa-let-7b_st | 11.82806599 | 0.842525676 | 12.82209662 | 0.677290735 |
| hsa-miR-623_st | 9.885798422 | 0.578155336 | 10.00710148 | 0.32620826 |
| hsa-miR-624_st | 3.969520828 | 0.422886781 | 4.261004215 | 0.314980347 |
| hsa-miR-624-star_st | 3.99343912 | 0.476864051 | 4.379409284 | 0.422719322 |
| hsa-miR-183_st | 5.058453265 | 0.971905538 | 6.982039097 | 1.259277558 |
| hsa-miR-30b_st | 11.59116391 | 1.198122831 | 12.69215784 | 0.779902862 |
| hsa-miR-626_st | 3.472680424 | 0.308422515 | 3.594160316 | 0.35465532 |
| hsa-miR-627_st | 3.129072514 | 0.539810849 | 3.576409299 | 0.385146715 |
| hsa-miR-628-3p_st | 11.47691285 | 0.579422553 | 11.45508435 | 0.615383402 |
| hsa-miR-31-star_st | 5.239646036 | 1.296229106 | 3.911476855 | 1.459314421 |
| hsa-miR-218-1-star_st | 4.062996958 | 0.285920264 | 3.98423594 | 0.630313349 |
| hsa-miR-629-star_st | 5.871066716 | 0.60748575 | 5.527233816 | 0.794129897 |
| hsa-miR-630_st | 8.938538143 | 0.977584959 | 8.806119351 | 0.595064206 |
| hsa-miR-631_st | 7.583534799 | 0.816214035 | 7.667707635 | 0.564391862 |
| hsa-miR-632_st | 4.916232896 | 0.800829888 | 4.822393076 | 0.606808758 |
| hsa-miR-633_st | 3.138674965 | 0.385893297 | 3.460051755 | 0.433740557 |
| hsa-miR-634_st | 6.258339453 | 0.620137849 | 6.240889141 | 1.272817359 |
| hsa-miR-635_st | 5.659248769 | 0.562681519 | 5.336673748 | 0.464412411 |
| hsa-miR-203_st | 6.423540288 | 1.084374656 | 7.439440382 | 2.171340814 |
| hsa-miR-331-3p_st | 8.472148194 | 1.187506452 | 10.23847105 | 0.646203126 |
| hsa-miR-125b_st | 13.09949383 | 0.702109457 | 14.20146519 | 0.464250863 |
| hsa-let-7i_st | 12.38948499 | 1.068896617 | 13.38923547 | 0.90753087 |
| hsa-miR-640_st | 6.219733565 | 0.794497856 | 5.873776145 | 0.601973187 |
| hsa-miR-641_st | 5.081677999 | 0.562050584 | 4.687074703 | 0.521783455 |
| hsa-miR-642_st | 4.223877565 | 0.272662971 | 4.160296476 | 0.366923815 |
| hsa-miR-29b_st | 11.01764744 | 0.888887242 | 11.7114879 | 1.190344904 |
| hsa-miR-122_st | 4.895684511 | 0.615743826 | 4.985849804 | 0.588096431 |
| hsa-miR-645_st | 4.965567166 | 0.681846899 | 4.379976944 | 0.425810106 |
| hsa-miR-646_st | 3.983426491 | 0.385495041 | 3.865908609 | 0.462377226 |
| hsa-miR-647_st | 5.055109578 | 0.279663437 | 5.076084146 | 0.579340545 |
| hsa-miR-648_st | 8.923372156 | 0.539030404 | 8.902512523 | 0.382869928 |
| hsa-miR-649_st | 4.624207587 | 0.685058185 | 4.075665045 | 0.456131096 |
| hsa-miR-182_st | 7.769010972 | 1.561712896 | 9.605644942 | 1.274144167 |
| hsa-miR-651_st | 3.482006648 | 0.157469598 | 3.406635002 | 0.439939363 |
| hsa-miR-652_st | 7.369480423 | 0.632957095 | 7.746298654 | 0.569445151 |
| hsa-miR-653_st | 3.076122594 | 0.514068141 | 3.31670527 | 0.59769199 |
| hsa-miR-654-3p_st | 4.614610453 | 0.720513685 | 4.355051691 | 0.504758262 |
| hsa-miR-374b_st | 6.394959207 | 1.182598946 | 7.183771368 | 1.711235299 |
| hsa-miR-130a_st | 10.08020103 | 0.766377135 | 10.35948611 | 0.984003351 |
| hsa-miR-656_st | 3.59368367 | 0.472311599 | 3.947381839 | 0.590640879 |
| hsa-miR-657_st | 4.78824282 | 0.50310201 | 4.180997774 | 0.448545847 |
| hsa-miR-658_st | 11.96276372 | 0.309542793 | 11.9505532 | 0.56871267 |
| hsa-miR-99b_st | 10.02021362 | 0.862994708 | 11.04204106 | 0.68261512 |
| hsa-miR-660_st | 7.246923416 | 1.367556434 | 7.487634613 | 1.430580082 |
| hsa-miR-661_st | 7.533247906 | 0.825409084 | 7.506004453 | 0.560404748 |
| hsa-miR-30e-star_st | 8.42191393 | 1.335578255 | 9.774091051 | 1.018720998 |
| hsa-miR-663_st | 15.28550632 | 0.571581079 | 15.80827195 | 0.407674765 |
| hsa-miR-663b_st | 10.65684266 | 0.592315565 | 10.46373313 | 0.5485667 |
| hsa-miR-664_st | 7.01565408 | 1.289861677 | 7.844233025 | 1.041008225 |
| hsa-miR-664-star_st | 10.0060307 | 1.053638776 | 10.59163044 | 0.377126646 |
| hsa-miR-30d_st | 11.25273078 | 1.214156415 | 12.48864482 | 0.640175272 |
| hsa-miR-668_st | 8.777045639 | 0.884135631 | 8.349651293 | 0.553983542 |
| hsa-miR-670_st | 5.747310405 | 0.87405109 | 5.440136642 | 0.492183582 |
| hsa-miR-671-3p_st | 7.584270412 | 0.260656901 | 7.080120327 | 0.695437942 |
| hsa-miR-671-5p_st | 12.35270782 | 0.454811243 | 12.42958886 | 0.478610556 |
| hsa-miR-30a-star_st | 8.899101909 | 1.483153656 | 9.976705262 | 1.063451556 |
| hsa-miR-675-star_st | 4.958203797 | 0.233639639 | 4.91946789 | 0.606874936 |
| hsa-miR-148b_st | 7.599713165 | 1.477317145 | 9.397418032 | 0.881095209 |
| hsa-miR-708_st | 7.193578542 | 0.633795806 | 7.917505926 | 1.763072643 |
| hsa-miR-708-star_st | 4.297842687 | 0.589554105 | 4.496346151 | 0.639644594 |
| hsa-miR-221-star_st | 6.853765937 | 0.886890319 | 6.279868601 | 1.986845527 |
| hsa-let-7e_st | 11.21243375 | 1.184807777 | 12.54330551 | 0.747295788 |
| hsa-miR-7-1-star_st | 6.095382778 | 0.838985946 | 5.766972965 | 1.186764016 |
| hsa-miR-27b_st | 11.22252658 | 1.330058545 | 12.22584587 | 1.067712522 |
| hsa-miR-34a_st | 8.563929477 | 1.30131094 | 9.368804664 | 1.531181708 |
| hsa-miR-744_st | 14.47352761 | 0.342781621 | 14.55719276 | 0.521324827 |
| hsa-miR-744-star_st | 5.129903524 | 0.401186334 | 5.18542505 | 0.736418494 |
| hsa-miR-92b_st | 7.757655051 | 0.621429065 | 8.64744807 | 0.847459277 |
| hsa-miR-759_st | 3.342634429 | 0.474872115 | 3.245981812 | 0.450738006 |
| hsa-miR-760_st | 10.31269004 | 0.338453418 | 10.33090223 | 0.410477449 |
| hsa-miR-92a_st | 9.860196166 | 0.874640451 | 0.66034641 | 0.60031385 |
| hsa-miR-335_st | 7.44065986 | 1.237982497 | 8.849757809 | 1.529777317 |
| hsa-miR-96_st | 4.916560095 | 1.726381766 | 6.782817325 | 1.392668119 |
| hsa-miR-95_st | 6.419503319 | 1.160882877 | 8.038699553 | 1.453109928 |
| hsa-miR-766_st | 8.121625224 | 1.169206144 | 7.444029713 | 0.490436233 |
| hsa-miR-767-3p_st | 4.399336486 | 0.451181211 | 4.356266772 | 0.58142475 |
| hsa-miR-767-5p_st | 4.281355069 | 0.569537805 | 4.293036544 | 0.383117083 |
| hsa-miR-98_st | 8.291406338 | 1.336101601 | 9.857443481 | 1.140926706 |
| hsa-miR-769-5p_st | 7.177465602 | 0.466270242 | 6.990061913 | 0.784687248 |
| hsa-miR-770-5p_st | 7.848862898 | 0.7354968 | 7.547105066 | 0.572219956 |
| hsa-miR-802_st | 2.820589581 | 0.503225295 | 3.168207515 | 0.596932763 |
| hsa-miR-34b-star_st | 4.884535734 | 1.330390012 | 5.785437325 | 1.094083237 |
| hsa-miR-581_st | 3.153942853 | 0.882257369 | 3.282757323 | 0.579800174 |
| hsa-miR-181d_st | 7.775845323 | 0.583324649 | 8.771388849 | 0.75547687 |
| hsa-miR-875-5p_st | 2.780704052 | 0.605992878 | 2.954163167 | 0.673656846 |
| hsa-miR-876-3p_st | 3.476204212 | 0.513375572 | 3.244079558 | 0.511027703 |
| hsa-miR-876-5p_st | 2.515085897 | 0.499140897 | 2.991209608 | 0.517939126 |
| hsa-miR-151-3p_st | 7.815485225 | 1.232437266 | 8.961126175 | 0.788320496 |
| hsa-miR-877-star_st | 6.836231238 | 0.750969254 | 6.233888885 | 0.649756636 |
| hsa-miR-885-3p_st | 13.17428473 | 0.372180383 | 13.35023301 | 0.523191421 |
| hsa-miR-200c_st | 11.72731562 | 1.146003666 | 13.54032501 | 0.628311183 |
| hsa-miR-429_st | 5.098363608 | 0.933323131 | 5.221627301 | 1.532553961 |
| hsa-miR-886-5p_st | 12.34586273 | 0.27548468 | 12.55590537 | 0.704659672 |
| hsa-miR-224_st | 5.699424326 | 0.463733371 | 6.820475407 | 1.296131582 |
| hsa-miR-888_st | 3.842608416 | 0.483434965 | 3.490770866 | 0.30311243 |
| hsa-miR-4321_st | 10.22451298 | 0.650138855 | 10.21041952 | 0.685719231 |
| hsa-miR-889_st | 3.607345442 | 0.389911157 | 3.648761784 | 0.419176963 |
| hsa-miR-181b_st | 8.89460766 | 0.440451072 | 9.950002863 | 0.865528583 |
| hsa-miR-891a_st | 6.877292691 | 0.505592784 | 6.907550631 | 0.792713463 |
| hsa-miR-891b_st | 3.438624537 | 0.53362213 | 4.002907223 | 0.360911427 |
| hsa-miR-892a_st | 4.704843624 | 0.944356777 | 4.65874597 | 0.61843953 |
| hsa-miR-892b_st | 5.006578747 | 0.597825949 | 4.727139299 | 0.596919606 |
| hsa-miR-9_st | 2.982215752 | 0.824162199 | 3.082438366 | 0.422994955 |
| hsa-miR-222_st | 11.62178498 | 0.926089153 | 11.48568956 | 2.126605516 |
| hsa-miR-141_st | 7.986905514 | 1.050589987 | 9.444311432 | 1.261764382 |
| hsa-miR-221_st | 11.60624812 | 0.988222465 | 11.81048476 | 2.19478178 |
| hsa-miR-924_st | 4.181057075 | 0.286787968 | 3.951915242 | 0.361943585 |
| hsa-miR-181a_st | 10.43765387 | 0.698446862 | 11.80267122 | 1.106291877 |
| hsa-miR-181c_st | 7.148352425 | 0.938790181 | 8.61671884 | 1.3557679 |
| hsa-miR-4324_st | 8.603306258 | 0.877013854 | 9.959830037 | 0.788616582 |
| hsa-miR-125a-5p_st | 11.75031192 | 0.767109329 | 13.35353695 | 0.62986375 |
| hsa-miR-92b-star_st | 14.57472977 | 0.39560879 | 14.83763365 | 0.444937351 |
| hsa-miR-3195_st | 13.30765887 | 0.582622655 | 13.32069034 | 0.718420244 |
| hsa-miR-3137_st | 11.58702289 | 0.836696507 | 11.62210382 | 0.443187468 |
| hsa-miR-934_st | 5.310817656 | 0.640263781 | 5.439073765 | 0.386455103 |
| hsa-miR-935_st | 4.623022642 | 0.886389105 | 4.704866773 | 0.37050234 |
| hsa-miR-551b_st | 3.766884015 | 0.279425944 | 4.130794671 | 1.129943036 |
| hsa-miR-937_st | 5.847801764 | 0.56164225 | 5.684689342 | 0.591690475 |
| hsa-miR-938_st | 4.932998476 | 0.731224911 | 4.946088502 | 0.515203533 |
| hsa-miR-939_st | 13.30913259 | 0.486002491 | 13.39199249 | 0.388159133 |
| hsa-miR-7-2-star_st | 5.128238135 | 0.876272281 | 7.414220509 | 1.685048772 |
| hsa-miR-146b-5p_st | 10.89707591 | 1.136970312 | 8.554021569 | 1.874250075 |
| hsa-miR-941_st | 5.920500651 | 0.548773823 | 5.822420998 | 0.35837617 |
| hsa-miR-942_st | 3.234496233 | 0.552133472 | 3.333967456 | 0.460742946 |
| hsa-miR-200a_st | 7.961812576 | 1.302057287 | 8.66965769 | 1.798567474 |
| hsa-miR-944_st | 3.377714124 | 0.567991126 | 3.599649798 | 0.526598467 |
| hsa-miR-7_st | 5.891833519 | 1.306520378 | 9.28147776 | 2.855280788 |
| hsa-miR-375_st | 5.491371894 | 0.284535933 | 5.116386036 | 0.888493672 |
| hsa-miR-96-star_st | 4.320346138 | 0.178619073 | 4.463952783 | 0.451769451 |
| hsa-miR-424_st | 6.750810254 | 1.251319367 | 8.245023788 | 1.319910967 |
| hsa-miR-181a-2-star_st | 6.495686121 | 1.145057647 | 6.932623147 | 1.227627845 |
| hsa-miR-200b_st | 8.841953131 | 1.654855811 | 10.16671313 | 1.530518629 |
| hsa-miR-146b-3p_st | 4.625036094 | 0.588004522 | 4.434495292 | 0.48125597 |
| hsa-miR-99b-star_st | 7.772704802 | 0.29446734 | 7.767958559 | 0.488768244 |
| hsa-miR-9-star_st | 4.860168706 | 0.643219089 | 4.051633994 | 0.728291835 |

| | Mean | STDEV |
|---|---|---|
| Sample Name | FTC | FTC |
| hsa-miR-150_st | 9.684397839 | 2.030200595 |
| hsa-let-7a-2-star_st | 4.801992143 | 0.40031824 |
| hsa-let-7a-star_st | 3.372863948 | 0.306870458 |
| hsa-miR-513b_st | 10.07239541 | 2.125055801 |
| hsa-let-7b-star_st | 7.521110163 | 1.210668934 |
| hsa-miR-593-star_st | 9.057241317 | 1.600550953 |
| hsa-let-7c-star_st | 5.197424112 | 0.716116771 |
| hsa-miR-516a-5p_st | 6.658268436 | 0.812874467 |
| hsa-miR-142-5p_st | 5.708297995 | 1.71919978 |
| hsa-miR-650_st | 11.08938602 | 0.39959731 |
| hsa-miR-513c_st | 7.291099373 | 1.373270189 |
| hsa-miR-761_st | 8.468850929 | 0.776150804 |
| hsa-let-7f-1-star_st | 5.22210362 | 0.445825202 |
| hsa-miR-513a-5p_st | 12.06848022 | 1.449363159 |
| hsa-miR-654-5p_st | 10.86471312 | 0.810883571 |
| hsa-let-7g-star_st | 4.851538847 | 0.382169199 |
| hsa-miR-3163_st | 8.450927076 | 0.793994625 |
| hsa-let-7i-star_st | 6.467173826 | 1.038849141 |
| hsa-miR-3165_st | 7.118819069 | 1.461449021 |
| hsa-miR-3194_st | 11.03308357 | 0.823888489 |
| hsa-miR-100-star_st | 3.11199991 | 0.622316476 |
| hsa-miR-4251_st | 6.441924053 | 0.885883403 |
| hsa-miR-101-star_st | 4.155607649 | 0.372058207 |
| hsa-miR-922_st | 6.063270798 | 0.86276452 |
| hsa-miR-103-2-star_st | 5.863656252 | 0.544295147 |
| hsa-miR-155_st | 9.543627212 | 1.471316722 |
| hsa-miR-105_st | 3.561756364 | 0.349405295 |
| hsa-miR-127-5p_st | 7.39917444 | 1.106211408 |
| hsa-miR-1286_st | 9.30499447 | 0.926152319 |
| hsa-miR-106a-star_st | 3.47848361 | 0.463543538 |
| hsa-miR-890_st | 6.84526332 | 1.196583479 |
| hsa-miR-3126-5p_st | 12.11210691 | 1.209546766 |
| hsa-miR-489_st | 9.726249388 | 1.316939018 |
| hsa-miR-10a_st | 8.559776736 | 1.511251806 |
| hsa-miR-10a-star_st | 4.667641659 | 0.258326618 |
| hsa-miR-10b_st | 9.952445986 | 1.3589001 |
| hsa-miR-10b-star_st | 5.059550184 | 0.693997383 |
| hsa-miR-1178_st | 4.496572393 | 0.480703396 |
| hsa-miR-1179_st | 3.486327382 | 0.457479467 |
| hsa-miR-1180_st | 9.227827435 | 0.337139575 |
| hsa-miR-1181_st | 9.369702547 | 0.658781274 |
| hsa-miR-3138_st | 10.78042744 | 1.23650974 |
| hsa-miR-220c_st | 6.492846656 | 0.731310485 |
| hsa-miR-510_st | 8.790855126 | 1.168963799 |
| hsa-miR-1185_st | 2.89975365 | 0.357709501 |
| hsa-miR-1193_st | 10.34571118 | 0.281969656 |
| hsa-miR-1197_st | 3.690595104 | 0.142039776 |
| hsa-miR-1200_st | 4.308937174 | 0.350211284 |
| hsa-miR-1201_st | 9.679543348 | 0.724633127 |
| hsa-miR-1202_st | 13.44070266 | 0.536454895 |
| hsa-miR-30c-1-star_st | 10.25428921 | 1.076886846 |
| hsa-miR-1204_st | 4.919279224 | 0.60261043 |
| hsa-miR-1205_st | 4.305331921 | 0.393146727 |
| hsa-miR-1206_st | 3.14827082 | 0.2500933 |
| hsa-miR-1207-3p_st | 4.176996676 | 0.520126092 |
| hsa-miR-1207-5p_st | 14.63438172 | 0.186223239 |
| hsa-miR-1208_st | 10.64451191 | 0.443782496 |
| hsa-miR-622_st | 9.076980683 | 0.757941588 |
| hsa-miR-1224-3p_st | 5.920681533 | 0.413151009 |
| hsa-miR-2116_st | 5.930670742 | 0.684583768 |
| hsa-miR-1225-3p_st | 6.297214822 | 0.319139341 |
| hsa-miR-1225-5p_st | 14.33743774 | 0.240262364 |
| hsa-miR-1226_st | 5.748037513 | 0.729821207 |
| hsa-miR-30b-star_st | 8.82906613 | 0.856938386 |
| hsa-miR-1227_st | 4.347000634 | 0.45037551 |
| hsa-miR-1295_st | 6.442125044 | 0.797678092 |
| hsa-miR-1228-star_st | 15.44532655 | 0.287252796 |
| hsa-miR-20b_st | 9.567595294 | 0.788174842 |
| hsa-miR-122-star_st | 3.275916843 | 0.390681148 |
| hsa-miR-1231_st | 14.30384357 | 0.322854194 |
| hsa-miR-1233_st | 6.146864348 | 0.527439621 |
| hsa-miR-524-5p_st | 5.617376015 | 0.437605645 |
| hsa-miR-1236_st | 6.156793888 | 0.318972586 |
| hsa-miR-873_st | 5.176832238 | 0.944395 |
| hsa-miR-1238_st | 7.318574027 | 0.283427483 |
| hsa-miR-124_st | 8.932397447 | 0.783450461 |
| hsa-miR-1243_st | 4.276596714 | 0.371460914 |
| hsa-miR-1244_st | 8.733595356 | 1.083387346 |
| hsa-miR-1245_st | 4.001232657 | 0.33931703 |
| hsa-miR-3122_st | 11.63630869 | 0.328402849 |
| hsa-miR-134_st | 9.883706365 | 0.610515281 |
| hsa-miR-1248_st | 10.69204902 | 1.568204691 |
| hsa-miR-146a_st | 8.888497102 | 1.487954143 |
| hsa-miR-124-star_st | 5.341966048 | 0.331174717 |
| hsa-miR-1250_st | 8.004089295 | 0.471851264 |
| hsa-miR-764_st | 5.457037176 | 0.811159414 |
| hsa-miR-1252_st | 3.21393874 | 0.37811015 |
| hsa-miR-223_st | 9.529322428 | 1.891176982 |
| hsa-miR-34c-3p_st | 8.229898667 | 0.847346814 |
| hsa-miR-1255a_st | 5.593509045 | 0.818559082 |
| hsa-miR-1255b_st | 8.167177678 | 1.29757817 |
| hsa-miR-1256_st | 3.872217782 | 0.428124334 |
| hsa-miR-1257_st | 5.236460925 | 0.506142245 |
| hsa-miR-199b-5p_st | 6.266646668 | 2.494363747 |
| hsa-miR-1259_st | 3.21314522 | 0.448890093 |
| hsa-miR-492_st | 8.999530322 | 0.441067934 |
| hsa-miR-3132_st | 9.678800325 | 0.650675187 |
| hsa-miR-516b_st | 6.973819553 | 1.06256649 |
| hsa-miR-4294_st | 8.691559725 | 1.067271513 |
| hsa-miR-125b-2-star_st | 10.37956176 | 1.054426987 |
| hsa-miR-126_st | 14.08130644 | 0.430060292 |
| hsa-miR-198_st | 12.33326178 | 0.828347642 |
| hsa-miR-3191_st | 11.85803443 | 0.920300444 |
| hsa-miR-1261_st | 7.535298754 | 1.088249212 |
| hsa-miR-1262_st | 4.302906304 | 0.516453445 |
| hsa-miR-1263_st | 4.468539056 | 0.528640803 |
| hsa-miR-1264_st | 3.461767589 | 0.490326851 |
| hsa-miR-1265_st | 5.503663375 | 0.500094805 |
| hsa-miR-2114-star_st | 7.16983379 | 1.192321836 |
| hsa-miR-1267_st | 4.150503304 | 0.267068704 |
| hsa-miR-1268_st | 14.25949219 | 0.191856461 |
| hsa-miR-1269_st | 5.938102341 | 0.526470159 |
| hsa-miR-509-5p_st | 6.081842306 | 0.792727654 |
| hsa-miR-1270_st | 6.494065335 | 0.632697671 |
| hsa-miR-1271_st | 8.045762953 | 0.631924217 |
| hsa-miR-4303_st | 6.840657169 | 0.793532317 |
| hsa-miR-1273_st | 10.01663585 | 0.864017561 |
| hsa-miR-583_st | 7.727106137 | 0.721974354 |
| hsa-miR-1273d_st | 10.75240388 | 0.507761118 |
| hsa-miR-127-3p_st | 6.448008252 | 1.803960093 |
| hsa-miR-522-star_st | 6.352051657 | 0.367469875 |
| hsa-miR-214-star_st | 5.39072323 | 1.448818619 |
| hsa-miR-184_st | 8.340582696 | 0.880349373 |
| hsa-miR-614_st | 6.30527271 | 1.061593282 |
| hsa-miR-526b_st | 5.962668217 | 0.426201988 |
| hsa-miR-1277_st | 3.297477828 | 0.256361604 |
| hsa-miR-1278_st | 4.05045819 | 0.422846181 |
| hsa-miR-1279_st | 3.365732577 | 0.417759882 |
| hsa-miR-409-3p_st | 6.441772661 | 1.162198954 |
| hsa-miR-199a-5p_st | 9.543793019 | 1.961495064 |
| hsa-miR-1281_st | 9.738350928 | 0.476501133 |
| hsa-miR-1282_st | 4.029630098 | 0.783351704 |
| hsa-miR-33b-star_st | 7.296620081 | 0.391831638 |
| hsa-miR-1284_st | 5.232096661 | 0.568826119 |
| hsa-miR-1253_st | 4.935285921 | 0.469735636 |
| hsa-miR-584_st | 7.627957116 | 0.701474559 |
| hsa-miR-1287_st | 6.444806627 | 0.420992295 |
| hsa-miR-15a_st | 11.20760903 | 0.607754854 |
| hsa-miR-1289_st | 4.709557118 | 0.717017416 |
| hsa-miR-1290_st | 10.8100822 | 0.839820881 |
| hsa-miR-2117_st | 3.882431528 | 0.457372323 |
| hsa-miR-1292_st | 12.57355094 | 0.37919776 |
| hsa-miR-1293_st | 7.427683447 | 0.295893299 |
| hsa-miR-129-3p_st | 3.842349929 | 0.518420222 |
| hsa-miR-520d-5p_st | 7.32057227 | 0.437487875 |
| hsa-miR-526a_st | 7.124328592 | 0.467985129 |
| hsa-miR-129-5p_st | 11.7877797 | 0.3716748 |
| hsa-miR-1254_st | 11.73016451 | 0.756583376 |
| hsa-miR-1297_st | 3.006529906 | 0.409742957 |
| hsa-miR-1298_st | 4.867614588 | 0.59670062 |
| hsa-miR-1299_st | 5.532663926 | 1.322641098 |
| hsa-miR-129-star_st | 4.319834478 | 0.533748872 |
| hsa-miR-1301_st | 8.642167863 | 0.177194896 |
| hsa-miR-19b_st | 10.64366877 | 0.495558783 |
| hsa-miR-1303_st | 10.70658683 | 0.426205016 |
| hsa-miR-1304_st | 7.670429896 | 0.921835503 |
| hsa-miR-1305_st | 4.076897822 | 0.417444591 |
| hsa-miR-550_st | 12.10356348 | 0.639005483 |
| hsa-miR-1307_st | 13.80208558 | 0.338179238 |
| hsa-miR-1308_st | 15.16215606 | 0.646017669 |
| hsa-miR-4291_st | 7.800841809 | 0.563278306 |
| hsa-miR-130a-star_st | 4.021740095 | 0.420688406 |
| hsa-miR-130b_st | 8.39229655 | 0.870958584 |
| hsa-miR-130b-star_st | 5.038512793 | 0.666110913 |
| hsa-miR-921_st | 11.65503813 | 0.877904978 |
| hsa-miR-520a-5p_st | 4.885617889 | 0.479530704 |
| hsa-miR-711_st | 12.14191487 | 0.510821702 |
| hsa-miR-1323_st | 8.517354804 | 0.45686443 |
| hsa-miR-1324_st | 3.351568117 | 0.562460357 |
| hsa-miR-490-3p_st | 6.123846509 | 0.41546502 |
| hsa-miR-718_st | 8.645398149 | 0.458718515 |
| hsa-miR-133b_st | 5.610531106 | 2.724024548 |
| hsa-miR-431-star_st | 6.553444872 | 0.300824565 |
| hsa-miR-552_st | 5.683650375 | 0.774719019 |
| hsa-miR-28-3p_st | 8.007495893 | 1.201147279 |
| hsa-miR-4265_st | 8.08795367 | 0.41000352 |
| hsa-miR-643_st | 3.976928251 | 0.558305835 |
| hsa-miR-136_st | 2.969563212 | 0.23345605 |
| hsa-miR-136-star_st | 3.128279102 | 0.36709184 |
| hsa-miR-137_st | 3.378087112 | 0.296252689 |
| hsa-miR-138_st | 9.992441972 | 1.61833596 |
| hsa-miR-138-1-star_st | 8.356146038 | 1.011487779 |
| hsa-miR-138-2-star_st | 5.448002704 | 0.426649171 |
| hsa-miR-139-3p_st | 8.17866992 | 0.522321927 |
| hsa-miR-139-5p_st | 7.71786577 | 1.490029753 |
| hsa-miR-140-3p_st | 10.94742817 | 0.797696947 |
| hsa-miR-140-5p_st | 6.540010051 | 0.999270696 |
| hsa-miR-381_st | 6.641994895 | 0.620288607 |
| hsa-miR-141-star_st | 3.799427769 | 0.408140134 |
| hsa-miR-17-star_st | 6.874398034 | 0.665879157 |
| hsa-miR-4300_st | 8.207999171 | 0.407383826 |
| hsa-miR-143_st | 12.3724 | 0.912433112 |
| hsa-miR-143-star_st | 6.748965421 | 1.106862092 |
| hsa-miR-144_st | 3.380806472 | 0.44073448 |
| hsa-miR-3198_st | 9.922115931 | 0.559358676 |
| hsa-miR-145_st | 12.96810302 | 1.224620382 |
| hsa-miR-145-star_st | 6.018243807 | 1.321094299 |
| hsa-miR-1468_st | 5.951634337 | 0.403696958 |
| hsa-miR-1469_st | 15.76481188 | 0.384176259 |
| hsa-miR-1973_st | 14.50158725 | 1.124348058 |
| hsa-miR-3154_st | 11.52524842 | 0.447345791 |
| hsa-miR-148a-star_st | 5.653785589 | 1.010896254 |
| hsa-miR-92a-1-star_st | 8.067286858 | 0.264280878 |
| hsa-miR-147_st | 4.025157603 | 0.518176196 |
| hsa-miR-194-star_st | 8.884856939 | 0.58032204 |
| hsa-miR-1471_st | 10.51673808 | 0.754633045 |
| hsa-miR-147b_st | 4.521029922 | 0.28223738 |
| hsa-miR-148a_st | 11.90540704 | 1.476361126 |
| hsa-miR-4299_st | 13.27685499 | 1.559239506 |
| hsa-miR-135a-star_st | 12.8429315 | 0.582365542 |
| hsa-miR-612_st | 11.82296472 | 0.536530807 |
| hsa-miR-1321_st | 7.644516768 | 0.492661734 |
| hsa-miR-149-star_st | 15.6720287 | 0.259673068 |
| hsa-miR-604_st | 8.975960499 | 1.169815451 |
| hsa-miR-150-star_st | 14.18390995 | 0.301402314 |
| hsa-miR-125b-1-star_st | 11.66287727 | 0.636846132 |
| hsa-miR-1183_st | 10.18594529 | 0.65309625 |
| hsa-miR-152_st | 10.94107803 | 0.766307302 |
| hsa-miR-153_st | 3.483394959 | 0.583311448 |
| hsa-miR-1537_st | 3.429463182 | 0.32274722 |
| hsa-miR-1538_st | 7.672519742 | 0.568179915 |
| hsa-miR-1539_st | 5.831050586 | 0.290805108 |
| hsa-miR-154_st | 4.453397304 | 1.051347967 |
| hsa-miR-154-star_st | 4.531144987 | 0.488827929 |
| hsa-miR-432-star_st | 4.861411452 | 0.651883684 |
| hsa-miR-155-star_st | 3.654999072 | 0.488749231 |
| hsa-miR-298_st | 10.8586502 | 0.603875865 |
| hsa-miR-15a-star_st | 4.690518246 | 0.591470645 |
| hsa-miR-2110_st | 10.9707848 | 0.766624212 |
| hsa-miR-15b-star_st | 3.706761596 | 0.291785894 |
| hsa-miR-1266_st | 9.510446132 | 0.512890477 |
| hsa-miR-16-1-star_st | 4.547784458 | 0.271903713 |
| hsa-miR-16-2-star_st | 4.675998573 | 0.832093841 |
| hsa-miR-490-5p_st | 7.554610413 | 0.62657975 |
| hsa-miR-19a_st | 6.530567548 | 0.912312856 |
| hsa-miR-3192_st | 9.047569026 | 0.510968073 |
| hsa-miR-3155_st | 7.720338464 | 0.563721376 |
| hsa-miR-187-star_st | 10.9811868 | 0.366948312 |
| hsa-miR-16_st | 14.00993192 | 0.495935174 |
| hsa-miR-936_st | 11.18337823 | 0.376217588 |
| hsa-miR-181c-star_st | 7.295464938 | 0.777034 |
| hsa-miR-379_st | 6.004420442 | 1.061999537 |
| hsa-miR-370_st | 11.04457054 | 0.439061503 |
| hsa-miR-541_st | 8.627429045 | 0.391486639 |
| hsa-miR-518c-star_st | 8.784237219 | 0.433117209 |
| hsa-miR-487b_st | 9.838051 | 0.858903274 |
| hsa-miR-182-star_st | 3.526728094 | 0.36626621 |
| hsa-miR-3121_st | 7.79081877 | 1.313522241 |
| hsa-miR-3190-5p_st | 10.38692172 | 0.418188044 |
| hsa-miR-1203_st | 9.181036937 | 0.521755032 |
| hsa-miR-185_st | 9.651865117 | 0.617785517 |
| hsa-miR-185-star_st | 9.507002775 | 0.523779387 |
| hsa-miR-4317_st | 9.89812612 | 0.580304623 |
| hsa-miR-186-star_st | 2.841188382 | 0.209005359 |
| hsa-miR-187_st | 5.728403231 | 1.088321407 |
| hsa-miR-4257_st | 12.39086919 | 0.544638489 |
| hsa-miR-188-3p_st | 4.835018522 | 0.65645238 |
| hsa-miR-1184_st | 5.089481247 | 0.381829557 |
| hsa-miR-18a_st | 5.197086592 | 0.573671424 |
| hsa-miR-18a-star_st | 5.172481891 | 0.544288689 |
| hsa-miR-3140_st | 5.315162277 | 0.58217134 |
| hsa-miR-18b-star_st | 4.259578437 | 0.37094237 |
| hsa-miR-190_st | 2.586472186 | 0.387319395 |
| hsa-miR-601_st | 5.937820377 | 0.663337894 |
| hsa-miR-1909_st | 14.73085745 | 0.334572678 |
| hsa-miR-342-5p_st | 5.975120939 | 1.136654656 |
| hsa-miR-575_st | 13.47370293 | 0.845807846 |
| hsa-miR-885-5p_st | 7.266016707 | 0.897742696 |
| hsa-miR-300_st | 6.150257496 | 0.434656204 |
| hsa-miR-1911_st | 4.238923243 | 0.337890538 |
| hsa-miR-1911-star_st | 5.614210775 | 0.424510492 |
| hsa-miR-1912_st | 4.585932615 | 0.394089715 |
| hsa-miR-4266_st | 6.953943916 | 0.383669601 |
| hsa-miR-1914_st | 6.003099294 | 0.457417748 |
| hsa-miR-3190-3p_st | 11.0379916 | 0.522051223 |
| hsa-miR-618_st | 3.961741747 | 0.347698399 |
| hsa-miR-1276_st | 7.347252958 | 0.631808791 |
| hsa-miR-191-star_st | 6.558696267 | 0.499301279 |
| hsa-miR-1258_st | 5.009724093 | 0.527690956 |
| hsa-miR-192-star_st | 3.994870165 | 0.31898997 |
| hsa-miR-193a-3p_st | 7.960929943 | 1.212842105 |
| hsa-miR-193a-5p_st | 12.8296714 | 0.453697391 |
| hsa-miR-3157_st | 5.209617259 | 0.598157691 |
| hsa-miR-193b-star_st | 11.15667668 | 0.340266123 |
| hsa-miR-194_st | 8.224233172 | 1.218904932 |
| hsa-miR-4307_st | 3.938445393 | 0.673436607 |
| hsa-miR-195_st | 11.95838606 | 1.068504483 |
| hsa-miR-610_st | 5.791236426 | 0.759668354 |
| hsa-miR-196a_st | 4.177120684 | 0.544313895 |
| hsa-miR-196a-star_st | 5.805146885 | 1.422744354 |
| hsa-miR-196b_st | 3.741124188 | 0.589615969 |
| hsa-miR-196b-star_st | 6.023379509 | 0.723406431 |
| hsa-let-7f-2-star_st | 3.928646853 | 0.296650067 |
| hsa-miR-1972_st | 12.47129182 | 0.617494416 |
| hsa-miR-3184_st | 10.27285548 | 0.641340634 |
| hsa-miR-523-star_st | 6.48935021 | 0.597801623 |
| hsa-miR-1976_st | 7.109527867 | 0.618703856 |
| hsa-miR-625-star_st | 4.342393386 | 0.333273099 |
| hsa-miR-587_st | 4.707223218 | 0.759769795 |
| hsa-miR-199a-3p_st | 11.00824548 | 1.770417008 |
| hsa-miR-1910_st | 8.849575972 | 0.311878349 |
| hsa-miR-199b-3p_st | 11.10214147 | 1.898031481 |
| hsa-miR-639_st | 8.797719601 | 0.445158939 |
| hsa-miR-1909-star_st | 8.802774604 | 0.537845378 |
| hsa-miR-19a-star_st | 3.271634952 | 0.525703123 |
| hsa-miR-29c-star_st | 8.568042505 | 0.762481744 |
| hsa-miR-940_st | 8.688476563 | 0.516037231 |
| hsa-miR-19b-2-star_st | 3.058340813 | 0.258856279 |
| hsa-miR-4269_st | 8.773844202 | 0.397071962 |
| hsa-miR-200a-star_st | 6.512464815 | 0.7518889 |
| hsa-miR-1182_st | 10.57116373 | 0.380926993 |
| hsa-miR-518f-star_st | 7.110157112 | 0.379465089 |
| hsa-miR-92a-2-star_st | 9.194527408 | 0.582851155 |
| hsa-miR-200c-star_st | 4.224332959 | 0.50157294 |
| hsa-miR-202_st | 8.999369422 | 0.549200301 |
| hsa-miR-202-star_st | 2.880805003 | 0.493296367 |
| hsa-miR-1914-star_st | 13.09615005 | 0.581606376 |
| hsa-miR-204_st | 7.537644966 | 2.50428894 |
| hsa-miR-665_st | 13.05111766 | 0.439684093 |
| hsa-miR-2052_st | 2.862672498 | 0.357151221 |
| hsa-miR-2053_st | 3.575832416 | 0.353705128 |
| hsa-miR-2054_st | 2.582416191 | 0.409813936 |
| hsa-miR-205-star_st | 5.595276649 | 0.477292948 |
| hsa-miR-206_st | 6.666957317 | 2.22261148 |
| hsa-miR-208a_st | 3.829837915 | 0.37206721 |
| hsa-miR-208b_st | 3.824268189 | 0.296514263 |
| hsa-miR-18b_st | 4.477775342 | 0.449974491 |
| hsa-miR-20a-star_st | 4.115724515 | 0.576766096 |
| hsa-miR-3181_st | 8.478858206 | 0.500184254 |
| hsa-miR-615-5p_st | 11.03969718 | 0.583072112 |
| hsa-miR-4259_st | 10.79380614 | 0.456419779 |
| hsa-miR-210_st | 12.80761013 | 0.204876062 |
| hsa-miR-211_st | 4.815518832 | 0.534354804 |
| hsa-miR-887_st | 9.495282895 | 0.290965419 |
| hsa-miR-2113_st | 5.095895681 | 0.483423186 |
| hsa-miR-2114_st | 4.402848711 | 0.709485505 |
| hsa-miR-943_st | 7.307454971 | 0.606519357 |
| hsa-miR-2115_st | 3.156025299 | 0.608268562 |
| hsa-miR-2115-star_st | 10.48337232 | 0.91397207 |
| hsa-miR-183-star_st | 7.451154522 | 0.403759852 |
| hsa-miR-2116-star_st | 4.147516168 | 0.234394382 |
| hsa-miR-920_st | 10.49642253 | 0.414405464 |
| hsa-miR-212_st | 6.826705809 | 0.623711158 |
| hsa-miR-214_st | 10.88969117 | 0.669950031 |
| hsa-miR-1825_st | 6.852608265 | 0.598211297 |
| hsa-miR-215_st | 6.19769958 | 1.011839518 |
| hsa-miR-216a_st | 3.87083365 | 0.433180844 |
| hsa-miR-195-star_st | 5.599806239 | 0.706978749 |
| hsa-miR-217_st | 3.475515731 | 0.19427291 |
| hsa-miR-23a-star_st | 10.96238378 | 0.338659258 |
| hsa-miR-25-star_st | 12.76757927 | 0.333093662 |
| hsa-miR-218-2-star_st | 3.96963769 | 0.337888222 |
| hsa-miR-1273c_st | 10.82936056 | 0.481170945 |
| hsa-miR-219-2-3p_st | 7.226891445 | 0.465828305 |
| hsa-miR-637_st | 11.97245562 | 0.33552103 |
| hsa-miR-21-star_st | 6.95176902 | 1.115364727 |
| hsa-miR-22_st | 12.29254594 | 0.852374387 |
| hsa-miR-220a_st | 3.393432707 | 0.317879002 |
| hsa-miR-220b_st | 4.11828969 | 0.294136579 |
| hsa-miR-219-1-3p_st | 5.825546478 | 0.545687734 |
| hsa-miR-4316_st | 7.700213681 | 0.386847562 |
| hsa-miR-877_st | 12.27522299 | 0.483116893 |
| hsa-miR-636_st | 7.511089328 | 0.415543686 |
| hsa-miR-3177_st | 12.01223967 | 0.417184451 |
| hsa-miR-1249_st | 5.154324867 | 0.81149596 |
| hsa-miR-223-star_st | 4.213405686 | 0.436928579 |
| hsa-miR-1224-5p_st | 13.56448668 | 0.283363968 |
| hsa-miR-564_st | 12.55409057 | 0.431169965 |
| hsa-miR-2276_st | 11.4073894 | 0.677146641 |
| hsa-miR-2277_st | 7.281358742 | 0.590818782 |
| hsa-miR-2278_st | 6.307746671 | 0.466857555 |
| hsa-miR-22-star_st | 8.717129888 | 1.108846566 |
| hsa-miR-2355_st | 4.339190991 | 0.259663072 |
| hsa-miR-769-3p_st | 9.635259227 | 0.649630558 |
| hsa-miR-1913_st | 7.819021132 | 0.527950377 |
| hsa-miR-1237_st | 7.277560013 | 0.562455124 |
| hsa-miR-1275_st | 13.61939169 | 0.411604954 |
| hsa-miR-30a_st | 13.27254252 | 0.680557429 |
| hsa-miR-20b-star_st | 5.873349963 | 0.791711391 |
| hsa-miR-24-2-star_st | 6.695415157 | 0.881327059 |
| hsa-miR-3173_st | 10.04382422 | 0.394268639 |
| hsa-miR-3148_st | 5.666403376 | 0.548228387 |
| hsa-miR-4271_st | 12.62482392 | 0.60660977 |
| hsa-miR-659_st | 10.80198836 | 0.268788515 |
| hsa-miR-26a-2-star_st | 3.075755776 | 0.344071646 |
| hsa-miR-1228_st | 7.131983608 | 0.528636384 |
| hsa-miR-26b-star_st | 5.662882666 | 0.754878165 |
| hsa-miR-3174_st | 7.46027396 | 0.882306121 |
| hsa-miR-27a-star_st | 5.877611888 | 0.843031573 |
| hsa-miR-1285_st | 12.81912566 | 0.460549845 |
| hsa-miR-483-5p_st | 12.00631979 | 0.380739147 |
| hsa-miR-1306_st | 6.854525564 | 0.473330343 |
| hsa-miR-431_st | 4.253451385 | 0.465403098 |
| hsa-miR-765_st | 9.327980594 | 0.221413986 |
| hsa-miR-2909_st | 4.007140779 | 0.460967497 |
| hsa-miR-296-3p_st | 11.35043933 | 0.281155734 |
| hsa-miR-296-5p_st | 4.851223384 | 0.532807028 |
| hsa-miR-297_st | 5.184118635 | 0.74662344 |
| hsa-miR-323b-5p_st | 7.581165298 | 0.550300621 |
| hsa-miR-299-3p_st | 7.24823859 | 0.601044849 |
| hsa-miR-299-5p_st | 4.301798986 | 1.575723707 |
| hsa-miR-487a_st | 6.390396571 | 0.459125596 |
| hsa-miR-29a-star_st | 3.385115056 | 0.343965916 |
| hsa-miR-625_st | 7.025496214 | 0.332652052 |
| hsa-miR-611_st | 8.397578281 | 0.478931198 |
| hsa-miR-3186-3p_st | 9.450485367 | 0.425993909 |
| hsa-miR-1294_st | 5.6148037 | 0.442298432 |
| hsa-miR-3147_st | 10.60149054 | 0.24154883 |
| hsa-miR-4285_st | 11.75170552 | 0.470308482 |
| hsa-miR-125a-3p_st | 11.68439475 | 0.391603862 |
| hsa-miR-301b_st | 3.190337513 | 0.469222559 |
| hsa-miR-302a_st | 3.371936124 | 0.350385734 |
| hsa-miR-518e-star_st | 7.036356274 | 0.293623841 |
| hsa-miR-302b_st | 2.791605682 | 0.326027493 |
| hsa-miR-302b-star_st | 4.072245283 | 0.562697047 |
| hsa-miR-302c_st | 3.144173714 | 0.521431056 |
| hsa-miR-302c-star_st | 9.105230572 | 0.526904953 |
| hsa-miR-302d_st | 3.098126626 | 0.458890549 |
| hsa-miR-302d-star_st | 8.666605122 | 0.709005932 |
| hsa-miR-302e_st | 3.47775912 | 0.50198292 |
| hsa-miR-302f_st | 3.280699617 | 0.465873032 |
| hsa-miR-3065-3p_st | 5.184459092 | 0.843557024 |
| hsa-miR-3130-5p_st | 7.050055234 | 0.627795578 |
| hsa-miR-3074_st | 5.249075451 | 0.291399661 |
| hsa-miR-1226-star_st | 11.86460707 | 0.299486483 |
| hsa-miR-3180-5p_st | 6.594980989 | 0.616951413 |
| hsa-miR-1229_st | 7.508303908 | 0.427858034 |
| hsa-miR-1272_st | 4.488833687 | 0.457340631 |
| hsa-miR-30c_st | 14.28937266 | 0.542874065 |
| hsa-miR-675_st | 13.5627101 | 0.421001419 |
| hsa-miR-30c-2-star_st | 7.579822359 | 0.746324753 |
| hsa-miR-1827_st | 4.895316041 | 0.437221839 |
| hsa-miR-30d-star_st | 3.891731196 | 0.616298388 |
| hsa-miR-30e_st | 10.92016909 | 0.773321641 |
| hsa-miR-1915-star_st | 7.349489776 | 0.481345943 |
| hsa-miR-1322_st | 4.23349115 | 0.592526476 |
| hsa-miR-3115_st | 3.610776151 | 0.396545906 |
| hsa-miR-3116_st | 7.208915115 | 1.078896161 |
| hsa-miR-3117_st | 4.435640351 | 0.627420383 |
| hsa-miR-3118_st | 3.715358324 | 0.266485825 |
| hsa-miR-519b-5p_st | 6.559554886 | 0.30156077 |
| hsa-miR-3120_st | 6.677804238 | 0.645550138 |
| hsa-miR-520d-3p_st | 5.576767903 | 0.73520043 |
| hsa-miR-326_st | 6.130531534 | 0.291784329 |
| hsa-miR-3123_st | 2.789237055 | 0.366699135 |
| hsa-miR-3124_st | 12.58558124 | 0.536882942 |
| hsa-miR-3125_st | 7.938228014 | 0.573509091 |
| hsa-miR-3126-3p_st | 4.666901922 | 0.502943863 |
| hsa-miR-1296_st | 6.938926305 | 0.293531727 |
| hsa-miR-3127_st | 8.975174453 | 0.843966032 |
| hsa-miR-3128_st | 3.930650243 | 0.479112795 |
| hsa-miR-3129_st | 4.12888939 | 0.362558573 |
| hsa-miR-3130-3p_st | 8.778599434 | 0.465272591 |
| hsa-miR-105-star_st | 3.708540946 | 0.312977026 |
| hsa-miR-3131_st | 11.99560471 | 0.277339966 |
| hsa-miR-4255_st | 4.107741512 | 0.389544232 |
| hsa-miR-3133_st | 4.216403911 | 0.877561247 |
| hsa-miR-3134_st | 3.418458437 | 0.321640675 |
| hsa-miR-3135_st | 6.941061293 | 0.54630175 |
| hsa-miR-3136_st | 6.008351283 | 0.457374169 |
| hsa-miR-19b-1-star_st | 4.258543503 | 0.282463819 |
| hsa-miR-1247_st | 7.2414891 | 0.640190428 |
| hsa-miR-3139_st | 4.12770938 | 0.24634358 |
| hsa-miR-596_st | 6.55040228 | 0.574760562 |
| hsa-miR-3141_st | 14.43383955 | 0.285458081 |
| hsa-miR-3142_st | 3.164110768 | 0.263262664 |
| hsa-miR-1302_st | 3.570000184 | 0.229394769 |
| hsa-miR-3144-3p_st | 4.471519188 | 0.354572452 |
| hsa-miR-3144-5p_st | 5.289990748 | 0.53647253 |
| hsa-miR-3145_st | 3.118116336 | 0.436308359 |
| hsa-miR-3146_st | 4.574188774 | 0.638085075 |
| hsa-miR-346_st | 8.035645876 | 0.246942258 |
| hsa-miR-4256_st | 3.82307943 | 0.555960757 |
| hsa-miR-3149_st | 4.626665044 | 0.539748912 |
| hsa-miR-3150_st | 9.64957915 | 0.417697005 |
| hsa-miR-3151_st | 11.68190953 | 0.296833232 |
| hsa-miR-1470_st | 5.516185771 | 0.370709765 |
| hsa-miR-3153_st | 9.315784053 | 0.469365186 |
| hsa-miR-514_st | 3.254056859 | 0.142908299 |
| hsa-miR-3175_st | 13.11212652 | 0.265290233 |
| hsa-miR-3156_st | 10.5851039 | 0.344476042 |
| hsa-miR-504_st | 5.833913206 | 0.595891154 |
| hsa-miR-3158_st | 4.83294665 | 0.73299485 |
| hsa-miR-3159_st | 5.200684285 | 0.552992624 |
| hsa-miR-3160_st | 5.795223213 | 0.365159196 |
| hsa-miR-3161_st | 8.155890392 | 0.391936212 |
| hsa-miR-3162_st | 13.49991321 | 0.438457983 |
| hsa-miR-1234_st | 7.201581226 | 0.61909594 |
| hsa-miR-3164_st | 7.554584823 | 0.868121021 |
| hsa-miR-516b-star_st | 3.986724903 | 0.423098861 |
| hsa-miR-3166_st | 4.777615587 | 0.33675287 |
| hsa-miR-4327_st | 12.62650526 | 0.242734875 |
| hsa-miR-3168_st | 3.370656466 | 0.455826435 |
| hsa-miR-3169_st | 4.43904408 | 0.479917343 |
| hsa-miR-3170_st | 6.825223335 | 0.69162506 |
| hsa-miR-3171_st | 3.384329204 | 0.321134498 |
| hsa-miR-3172_st | 15.29627133 | 0.544701056 |
| hsa-miR-374b-star_st | 4.017231016 | 0.586501073 |
| hsa-miR-34c-5p_st | 4.089525444 | 0.299664156 |
| hsa-miR-758_st | 3.683335986 | 1.239779546 |
| hsa-miR-3176_st | 11.74264074 | 0.399727585 |
| hsa-miR-606_st | 3.226613314 | 0.257855175 |
| hsa-miR-3178_st | 15.30976881 | 0.227856895 |
| hsa-miR-3179_st | 4.344222837 | 0.466410272 |
| hsa-miR-3180-3p_st | 14.57238259 | 0.444723077 |
| hsa-miR-597_st | 3.442837033 | 0.391655326 |
| hsa-miR-148b-star_st | 3.566299726 | 0.477039028 |
| hsa-miR-3182_st | 4.085121177 | 0.685616918 |
| hsa-miR-3183_st | 5.812834186 | 0.436499199 |
| hsa-miR-144-star_st | 3.908795092 | 0.53467528 |
| hsa-miR-3185_st | 15.03206059 | 0.306509224 |
| hsa-miR-4270_st | 15.09579795 | 0.21016673 |
| hsa-miR-3186-5p_st | 5.390808732 | 0.46719134 |
| hsa-miR-3187_st | 13.18360219 | 0.327707693 |
| hsa-miR-3188_st | 13.74314582 | 0.273936388 |
| hsa-miR-3189_st | 7.737779353 | 0.502733617 |
| hsa-miR-548b-3p_st | 3.556341108 | 0.710390995 |
| hsa-miR-99a-star_st | 4.912698607 | 1.3043586 |
| hsa-miR-376a-star_st | 2.685285427 | 0.443857371 |
| hsa-miR-216b_st | 3.95727231 | 0.37069765 |
| hsa-miR-3193_st | 6.746882159 | 0.553095246 |
| hsa-miR-548v_st | 3.133453372 | 0.212423805 |
| hsa-miR-26a-1-star_st | 3.338966246 | 0.134812502 |
| hsa-miR-1291_st | 9.084592289 | 0.971269335 |
| hsa-miR-3197_st | 14.93271548 | 0.511207753 |
| hsa-miR-629_st | 8.065266723 | 0.534837224 |
| hsa-miR-3199_st | 5.506277433 | 0.837744071 |
| hsa-miR-190b_st | 3.128437569 | 0.360713843 |
| hsa-miR-32_st | 3.239551137 | 0.513190588 |
| hsa-miR-3200_st | 4.547925804 | 0.906615209 |
| hsa-miR-3201_st | 4.043169762 | 0.32963962 |
| hsa-miR-3202_st | 7.912544662 | 0.468675387 |
| hsa-miR-320a_st | 12.4225901 | 0.401636292 |
| hsa-miR-4289_st | 7.078831223 | 0.722593395 |
| hsa-miR-655_st | 3.151488593 | 0.300066058 |
| hsa-miR-320b_st | 11.53346618 | 0.619793277 |
| hsa-miR-219-5p_st | 3.208878832 | 0.60694573 |
| hsa-miR-323-3p_st | 4.252677406 | 0.419297224 |
| hsa-miR-323-5p_st | 10.86068759 | 0.531035149 |
| hsa-miR-323b-3p_st | 3.866854724 | 0.429435637 |
| hsa-miR-103-as_st | 4.21398918 | 0.353107482 |
| hsa-miR-424-star_st | 8.553265843 | 0.569649486 |
| hsa-miR-33a-star_st | 4.451078454 | 0.902130275 |
| hsa-miR-325_st | 4.712972626 | 0.62508469 |
| hsa-miR-590-3p_st | 2.993479219 | 0.225401655 |
| hsa-miR-328_st | 8.312749571 | 1.535826243 |
| hsa-miR-329_st | 4.2966557 | 1.156107572 |
| hsa-miR-32-star_st | 4.783096994 | 0.422984744 |
| hsa-miR-330-3p_st | 6.893797967 | 0.799702999 |
| hsa-miR-330-5p_st | 5.928273833 | 0.516244273 |
| hsa-miR-146a-star_st | 3.206850686 | 0.407472745 |
| hsa-miR-331-5p_st | 7.398753321 | 0.497356019 |
| hsa-miR-3167_st | 3.045504056 | 0.324089167 |
| hsa-miR-335-star_st | 3.844847555 | 0.652327574 |
| hsa-miR-337-3p_st | 4.199664623 | 0.631295886 |
| hsa-miR-337-5p_st | 4.057207561 | 0.38343855 |
| hsa-miR-421_st | 7.942237641 | 0.57065684 |
| hsa-miR-338-5p_st | 5.886659545 | 0.725039099 |
| hsa-miR-339-3p_st | 9.051177663 | 0.399506814 |
| hsa-miR-548a-5p_st | 4.670450617 | 0.323197334 |
| hsa-miR-33a_st | 2.892415799 | 0.277350782 |
| hsa-miR-590-5p_st | 3.953177377 | 0.523480909 |
| hsa-miR-33b_st | 3.402609732 | 0.291359289 |
| hsa-miR-616-star_st | 4.139337241 | 0.5918454 |
| hsa-miR-569_st | 3.498514273 | 0.320608046 |
| hsa-miR-340-star_st | 3.210560655 | 0.582122833 |
| hsa-miR-342-3p_st | 11.2488144 | 1.16502565 |
| hsa-miR-135b-star_st | 8.240680243 | 0.565953941 |
| hsa-miR-345_st | 10.95286752 | 1.668060341 |
| hsa-miR-1288_st | 4.928072864 | 0.373435624 |
| hsa-miR-135a_st | 2.867734151 | 0.473745911 |
| hsa-miR-34a-star_st | 6.052989514 | 1.225371129 |
| hsa-miR-34b_st | 5.431001289 | 0.882879925 |
| hsa-miR-4288_st | 4.644021522 | 0.580338975 |
| hsa-miR-222-star_st | 6.562350775 | 1.245906925 |
| hsa-miR-933_st | 11.90411852 | 0.796510931 |
| hsa-miR-361-3p_st | 7.013604761 | 0.911944256 |
| hsa-miR-224-star_st | 6.153774762 | 0.871977349 |
| hsa-miR-362-3p_st | 5.32350941 | 1.079941835 |
| hsa-miR-362-5p_st | 7.490249612 | 1.005269378 |
| hsa-miR-363_st | 6.620856034 | 1.849678668 |
| hsa-miR-363-star_st | 9.610751036 | 0.522233527 |
| hsa-miR-1979_st | 15.4413111 | 0.495347856 |
| hsa-miR-365-star_st | 12.08774363 | 0.2897168 |
| hsa-miR-548x_st | 2.782963003 | 0.197377435 |
| hsa-miR-367-star_st | 3.183331613 | 0.457920997 |
| hsa-miR-369-3p_st | 2.961608618 | 0.513217185 |
| hsa-miR-369-5p_st | 4.277538763 | 1.047812939 |
| hsa-miR-548l_st | 4.953185936 | 0.396990376 |
| hsa-miR-371-3p_st | 3.741146864 | 0.32346407 |
| hsa-miR-371-5p_st | 12.74275184 | 0.234540689 |
| hsa-miR-372_st | 4.000273901 | 0.573003016 |
| hsa-miR-373_st | 5.437062044 | 0.514256568 |
| hsa-miR-373-star_st | 12.10740228 | 0.364518949 |
| hsa-miR-374a_st | 5.649003302 | 1.335902823 |
| hsa-miR-374a-star_st | 3.89452037 | 0.396409833 |
| hsa-miR-500_st | 6.6798214 | 0.992860143 |
| hsa-miR-320c_st | 11.1652493 | 0.693041535 |
| hsa-miR-875-3p_st | 4.40886737 | 0.222072564 |
| hsa-miR-376a_st | 4.684874048 | 1.151667552 |
| hsa-miR-29b-1-star_st | 7.279226499 | 0.678571346 |
| hsa-miR-376b_st | 3.707404461 | 0.914756727 |
| hsa-miR-376c_st | 4.908382893 | 1.758459958 |
| hsa-miR-377_st | 3.341287444 | 0.942733943 |
| hsa-miR-377-star_st | 4.931630073 | 0.64737812 |
| hsa-miR-378_st | 9.213482306 | 1.741952188 |
| hsa-miR-378b_st | 9.077366988 | 0.413977109 |
| hsa-miR-378c_st | 9.718320464 | 1.18930087 |
| hsa-miR-378-star_st | 6.030470904 | 1.094828166 |
| hsa-miR-662_st | 7.774829608 | 0.51388285 |
| hsa-miR-379-star_st | 3.559986749 | 0.409320419 |
| hsa-miR-380_st | 3.330200012 | 0.378690677 |
| hsa-miR-380-star_st | 3.711996377 | 0.322691633 |
| hsa-miR-1274b_st | 14.94058828 | 0.54391363 |
| hsa-miR-382_st | 6.652524581 | 1.349296826 |
| hsa-miR-383_st | 6.141995543 | 0.493214782 |
| hsa-miR-384_st | 2.996086095 | 0.403925048 |
| hsa-miR-126-star_st | 3.411377342 | 0.368451784 |
| hsa-miR-409-5p_st | 4.85181071 | 0.707204813 |
| hsa-miR-410_st | 4.498865745 | 0.516632017 |
| hsa-miR-411_st | 4.576889852 | 0.68763499 |
| hsa-miR-411-star_st | 4.561423335 | 0.706340387 |
| hsa-miR-412_st | 4.791947723 | 0.409720832 |
| hsa-miR-3143_st | 2.941950271 | 0.318295492 |
| hsa-miR-422a_st | 10.34599866 | 1.090694276 |
| hsa-miR-628-5p_st | 3.784574225 | 0.506457459 |
| hsa-miR-423-5p_st | 13.43878403 | 0.248437657 |
| hsa-miR-27b-star_st | 7.355607354 | 0.571966205 |
| hsa-miR-499-5p_st | 4.407003916 | 0.718004277 |
| hsa-miR-425_st | 9.0873123 | 1.160485222 |
| hsa-miR-193b_st | 9.166544235 | 1.522180683 |
| hsa-miR-4252_st | 5.343908951 | 0.220163684 |
| hsa-miR-4253_st | 12.40901451 | 0.355129942 |
| hsa-miR-4254_st | 5.88972572 | 0.760154385 |
| hsa-miR-324-3p_st | 8.638252103 | 0.614347755 |
| hsa-miR-4329_st | 5.540145084 | 0.475494365 |
| hsa-miR-1251_st | 5.276138327 | 1.272451509 |
| hsa-miR-4258_st | 5.739429435 | 0.595331953 |
| hsa-miR-1274a_st | 12.01378243 | 1.086884889 |
| hsa-miR-425-star_st | 8.311401133 | 0.443205807 |
| hsa-miR-4260_st | 11.09959526 | 0.401846993 |
| hsa-miR-4261_st | 9.091270887 | 0.444470782 |
| hsa-miR-4262_st | 3.153495546 | 0.283272641 |
| hsa-miR-4263_st | 3.385939651 | 0.341158591 |
| hsa-miR-4264_st | 3.001896818 | 0.408293862 |
| hsa-miR-1280_st | 15.13535033 | 0.52217549 |
| hsa-miR-132-star_st | 4.907042507 | 1.763889342 |
| hsa-miR-4267_st | 6.71640433 | 0.626513026 |
| hsa-miR-4268_st | 3.444763631 | 0.520167354 |
| hsa-miR-133a_st | 5.873173908 | 2.632190041 |
| hsa-miR-888-star_st | 3.27835693 | 0.388910787 |
| hsa-miR-1260b_st | 15.3234836 | 0.505237198 |
| hsa-miR-4272_st | 3.406672131 | 0.305244513 |
| hsa-miR-4273_st | 3.239080082 | 0.369009845 |
| hsa-miR-4274_st | 5.398199373 | 0.714183061 |
| hsa-miR-4275_st | 3.350668526 | 0.386389659 |
| hsa-miR-4276_st | 3.156190599 | 0.301541503 |
| hsa-miR-4277_st | 3.773495107 | 0.401206613 |
| hsa-miR-4278_st | 7.268474565 | 0.564502846 |
| hsa-miR-4279_st | 5.208488961 | 0.51609988 |
| hsa-miR-4280_st | 4.686043367 | 0.603816913 |
| hsa-miR-4281_st | 15.66461547 | 0.261815898 |
| hsa-miR-4282_st | 2.744196394 | 0.26549633 |
| hsa-miR-4283_st | 8.659351553 | 0.326215527 |
| hsa-miR-4284_st | 15.49872761 | 0.490125212 |
| hsa-miR-23b-star_st | 6.13333434 | 0.736120384 |
| hsa-miR-574-3p_st | 9.749266604 | 0.741289817 |
| hsa-miR-4287_st | 5.863355796 | 0.582559867 |
| hsa-miR-320e_st | 10.32594704 | 0.763317095 |
| hsa-miR-339-5p_st | 8.661053114 | 1.163828351 |
| hsa-miR-505_st | 6.621666555 | 1.325958275 |
| hsa-miR-4290_st | 6.200703582 | 0.460321716 |
| hsa-miR-200b-star_st | 6.679871859 | 0.773850547 |
| hsa-miR-4292_st | 8.19748633 | 0.557619175 |
| hsa-miR-4293_st | 5.189029436 | 0.479610732 |
| hsa-miR-1246_st | 13.68095299 | 0.681247365 |
| hsa-miR-4295_st | 3.65522638 | 0.504157094 |
| hsa-miR-4296_st | 7.124150097 | 0.345653387 |
| hsa-miR-4297_st | 5.468155664 | 0.74084134 |
| hsa-miR-4298_st | 13.13573126 | 0.399912879 |
| hsa-miR-218_st | 6.61199358 | 1.259616781 |
| hsa-miR-4286_st | 14.8652516 | 0.578878017 |
| hsa-miR-4301_st | 3.026225561 | 0.564332703 |
| hsa-miR-4302_st | 5.97448935 | 0.439315791 |
| hsa-miR-365_st | 5.176835655 | 1.246394081 |
| hsa-miR-4304_st | 6.871337147 | 0.742003523 |
| hsa-miR-4305_st | 3.787640926 | 0.531944805 |
| hsa-miR-4306_st | 9.535983026 | 0.51714214 |
| hsa-miR-26a_st | 14.90695899 | 0.51156795 |
| hsa-miR-4308_st | 3.78880468 | 0.607563341 |
| hsa-miR-4309_st | 4.31535144 | 0.26428961 |
| hsa-miR-186_st | 7.080516201 | 1.409673502 |
| hsa-miR-4310_st | 4.973343496 | 0.890987577 |
| hsa-miR-4311_st | 5.693961698 | 0.669278087 |
| hsa-miR-4312_st | 6.264058944 | 0.738454049 |
| hsa-miR-4313_st | 5.887436429 | 0.464580421 |
| hsa-miR-4314_st | 9.119900523 | 1.083326925 |
| hsa-miR-4315_st | 4.290008742 | 0.554097088 |
| hsa-miR-149_st | 7.39185902 | 1.226830426 |
| hsa-miR-106b-star_st | 7.915337616 | 0.913391598 |
| hsa-miR-4318_st | 4.6700349 | 0.347001437 |
| hsa-miR-4319_st | 4.928529172 | 0.746914154 |
| hsa-miR-29b-2-star_st | 8.227489531 | 0.536591921 |
| hsa-miR-432_st | 7.631254772 | 0.811761489 |
| hsa-miR-4320_st | 4.223809169 | 0.323386139 |
| hsa-miR-522_st | 3.703969101 | 0.280449254 |
| hsa-miR-4322_st | 13.0863392 | 0.256866791 |
| hsa-miR-4323_st | 5.167897357 | 0.482119592 |
| hsa-miR-93-star_st | 5.994440085 | 0.489588535 |
| hsa-miR-4325_st | 3.488043738 | 0.311391584 |
| hsa-miR-4326_st | 4.808428873 | 0.599008148 |
| hsa-miR-874_st | 12.8461055 | 0.480470556 |
| hsa-miR-4328_st | 3.26785648 | 0.296088316 |
| hsa-let-7e-star_st | 4.963051024 | 0.725530023 |
| hsa-miR-720_st | 15.7483214 | 0.419532142 |
| hsa-miR-433_st | 5.947768346 | 0.337722691 |
| hsa-miR-4330_st | 4.965598442 | 0.612856432 |
| hsa-miR-448_st | 3.86226093 | 0.278933369 |
| hsa-miR-449a_st | 4.241910625 | 0.717370749 |
| hsa-miR-449b_st | 5.170002335 | 0.411848857 |
| hsa-miR-1975_st | 15.77186527 | 0.411917838 |
| hsa-miR-449c_st | 7.071390053 | 0.383437615 |
| hsa-miR-449c-star_st | 4.264993111 | 0.611575968 |
| hsa-miR-1_st | 5.376074978 | 2.305750379 |
| hsa-miR-450b-3p_st | 2.688212847 | 0.249713618 |
| hsa-miR-450b-5p_st | 2.822577189 | 0.439570045 |
| hsa-miR-451_st | 13.39321695 | 1.550064198 |
| hsa-miR-452_st | 7.290523105 | 0.70692095 |
| hsa-miR-452-star_st | 3.649406763 | 0.281861009 |
| hsa-miR-548b-5p_st | 3.871467248 | 0.507449017 |
| hsa-miR-454-star_st | 3.936818624 | 0.585606308 |
| hsa-miR-455-3p_st | 8.847518872 | 1.327857022 |
| hsa-miR-455-5p_st | 5.120472664 | 1.216879325 |
| hsa-miR-466_st | 5.579335548 | 0.407555273 |
| hsa-miR-483-3p_st | 6.507073942 | 0.493416598 |
| hsa-miR-17_st | 10.79610458 | 0.755784015 |
| hsa-miR-320d_st | 10.4789272 | 0.876615473 |
| hsa-miR-485-3p_st | 6.160627625 | 0.847401937 |
| hsa-miR-485-5p_st | 7.303097824 | 0.346895336 |
| hsa-miR-486-3p_st | 10.67965777 | 0.594364678 |
| hsa-miR-486-5p_st | 8.18062968 | 1.812652996 |
| hsa-miR-644_st | 3.149911878 | 0.41232815 |
| hsa-miR-3152_st | 7.21844206 | 0.368349442 |
| hsa-miR-488_st | 3.560412911 | 0.395003544 |
| hsa-miR-488-star_st | 3.599494801 | 0.284920054 |
| hsa-miR-450a_st | 5.11867146 | 1.164158376 |
| hsa-miR-20a_st | 10.62204636 | 0.818243118 |
| hsa-miR-484_st | 8.895203829 | 1.033261644 |
| hsa-miR-491-3p_st | 4.678098205 | 0.561351009 |
| hsa-miR-491-5p_st | 9.136942032 | 0.64798363 |
| hsa-miR-197_st | 7.661304009 | 1.307684561 |
| hsa-miR-493_st | 6.020209593 | 0.570452517 |
| hsa-miR-493-star_st | 4.80526975 | 1.317850989 |
| hsa-miR-494_st | 14.99647551 | 0.939752818 |
| hsa-miR-495_st | 4.484255343 | 1.226010485 |
| hsa-miR-496_st | 3.160833855 | 0.364338261 |
| hsa-miR-497_st | 9.471691257 | 1.226313719 |
| hsa-miR-497-star_st | 3.400323936 | 0.282893889 |
| hsa-miR-498_st | 12.19131735 | 0.442767078 |
| hsa-miR-499-3p_st | 4.058592597 | 0.234983218 |
| hsa-miR-24-1-star_st | 4.718988758 | 0.616138227 |
| hsa-miR-340_st | 6.796638731 | 1.334138597 |
| hsa-miR-500b_st | 5.262278681 | 0.56958521 |
| hsa-miR-500-star_st | 9.569492631 | 0.380664132 |
| hsa-miR-501-3p_st | 9.379328872 | 0.218445155 |
| hsa-miR-501-5p_st | 6.332764202 | 0.617290949 |
| hsa-miR-502-3p_st | 9.416801105 | 0.42692491 |
| hsa-miR-502-5p_st | 5.380939006 | 0.418495834 |
| hsa-miR-503_st | 10.80830664 | 0.623673196 |
| hsa-miR-192_st | 8.541965238 | 1.409131387 |
| hsa-let-7g_st | 14.06382374 | 0.39565784 |
| hsa-miR-505-star_st | 7.78133659 | 0.346535229 |
| hsa-miR-506_st | 3.673161271 | 0.49318153 |
| hsa-miR-507_st | 3.404246303 | 0.515044482 |
| hsa-miR-508-3p_st | 3.074584567 | 0.309106647 |
| hsa-miR-508-5p_st | 9.264666089 | 0.429555275 |
| hsa-miR-509-3-5p_st | 6.81418112 | 0.775902279 |
| hsa-miR-509-3p_st | 5.995533286 | 0.54022872 |
| hsa-miR-106a_st | 10.69222748 | 0.77314535 |
| hsa-miR-106b_st | 10.08163403 | 0.69581812 |
| hsa-miR-511_st | 3.158940863 | 0.292177578 |
| hsa-miR-512-3p_st | 4.999182777 | 0.350162879 |
| hsa-miR-512-5p_st | 6.040161487 | 0.512190591 |
| hsa-miR-513a-3p_st | 2.923100784 | 0.572540564 |
| hsa-miR-23a_st | 14.37856638 | 0.6676877 |
| hsa-miR-25_st | 11.30756239 | 0.882723634 |
| hsa-miR-598_st | 6.869773958 | 0.918089686 |
| hsa-miR-1260_st | 12.05840004 | 0.703605507 |
| hsa-miR-514b-3p_st | 3.699491779 | 0.550667041 |
| hsa-miR-514b-5p_st | 11.35349145 | 0.280259946 |
| hsa-miR-515-3p_st | 3.979624719 | 0.525996601 |
| hsa-miR-515-5p_st | 3.505655154 | 0.343457528 |
| hsa-miR-516a-3p_st | 3.715244151 | 0.466843095 |
| hsa-miR-27a_st | 12.92470244 | 0.577313802 |
| hsa-miR-100_st | 12.77173856 | 1.020204874 |
| hsa-miR-101_st | 6.829956399 | 1.297156336 |
| hsa-miR-517a_st | 3.333133205 | 0.5668127 |
| hsa-miR-517b_st | 3.775604191 | 0.496678305 |
| hsa-miR-517c_st | 2.721664544 | 0.363523875 |
| hsa-miR-517-star_st | 5.450587185 | 0.563151152 |
| hsa-miR-518a-3p_st | 3.559509636 | 0.281367776 |
| hsa-miR-518a-5p_st | 7.434671786 | 0.526800511 |
| hsa-miR-518b_st | 4.012035822 | 0.568792387 |
| hsa-miR-518c_st | 3.068738546 | 0.244250183 |
| hsa-miR-301a_st | 4.998977439 | 0.762131676 |
| hsa-miR-518d-3p_st | 4.038622183 | 0.289602228 |
| hsa-miR-518d-5p_st | 7.25001624 | 0.458017574 |
| hsa-miR-518e_st | 2.970039356 | 0.445043612 |
| hsa-let-7f_st | 14.91679179 | 0.387254065 |
| hsa-miR-518f_st | 3.083099789 | 0.449826696 |
| hsa-miR-1826_st | 15.75362922 | 0.310081871 |
| hsa-miR-519a_st | 2.849666716 | 0.327335175 |
| hsa-miR-519a-star_st | 6.384645207 | 0.492776394 |
| hsa-miR-519b-3p_st | 3.277287738 | 0.426171255 |
| hsa-miR-1283_st | 3.504605506 | 0.305024538 |
| hsa-miR-519c-3p_st | 2.517410105 | 0.522108747 |
| hsa-miR-519c-5p_st | 6.734916687 | 0.563317311 |
| hsa-miR-519d_st | 3.617809075 | 0.342104697 |
| hsa-miR-519e_st | 3.988636353 | 0.572580782 |
| hsa-miR-519e-star_st | 7.342054981 | 0.236977546 |
| hsa-miR-520a-3p_st | 3.596810867 | 0.494428604 |
| hsa-miR-1908_st | 15.8041379 | 0.414691424 |
| hsa-miR-520b_st | 3.353519816 | 0.308070255 |
| hsa-miR-520c-3p_st | 4.303511217 | 0.356594871 |
| hsa-miR-520c-5p_st | 7.224306828 | 0.498754717 |
| hsa-miR-181a-star_st | 6.691426079 | 1.13206964 |
| hsa-miR-21_st | 12.92274273 | 1.239749919 |
| hsa-miR-520e_st | 3.229268256 | 0.387340715 |
| hsa-miR-520f_st | 3.719470114 | 0.431891488 |
| hsa-miR-520g_st | 3.684153879 | 0.660830115 |
| hsa-miR-520h_st | 3.64041064 | 0.374876326 |
| hsa-miR-521_st | 3.60274436 | 0.492597027 |
| hsa-miR-302a-star_st | 3.651475183 | 0.294143152 |
| hsa-miR-886-3p_st | 11.953639 | 1.384627362 |
| hsa-miR-523_st | 3.177186065 | 0.520925895 |
| hsa-miR-29a_st | 14.1920979 | 0.626105259 |
| hsa-miR-524-3p_st | 4.011026282 | 0.234269537 |
| hsa-miR-142-3p_st | 2.751842708 | 0.284039947 |
| hsa-miR-525-3p_st | 3.273198841 | 0.471986861 |
| hsa-miR-525-5p_st | 6.803250978 | 0.454968577 |
| hsa-miR-762_st | 15.78238967 | 0.364665001 |
| hsa-miR-423-3p_st | 9.96946924 | 0.686029368 |
| hsa-miR-449b-star_st | 7.803500892 | 0.51912185 |
| hsa-miR-527_st | 7.685628011 | 0.56673285 |
| hsa-miR-532-3p_st | 8.434978909 | 0.860766793 |
| hsa-miR-532-5p_st | 8.679214426 | 0.741788538 |
| hsa-miR-539_st | 4.481365975 | 0.787647433 |
| hsa-let-7a_st | 14.87045973 | 0.444864419 |
| hsa-miR-541-star_st | 6.464029154 | 0.34654011 |
| hsa-miR-542-3p_st | 7.191838838 | 1.403507336 |
| hsa-miR-542-5p_st | 7.966147356 | 0.456099059 |
| hsa-miR-543_st | 5.199151705 | 0.903256355 |
| hsa-miR-544_st | 2.881950092 | 0.240300269 |
| hsa-miR-544b_st | 3.871452407 | 0.212950363 |
| hsa-miR-545_st | 3.102771856 | 0.473250838 |
| hsa-miR-545-star_st | 2.651760132 | 0.542719342 |
| hsa-miR-548a-3p_st | 2.645582337 | 0.536749311 |
| hsa-miR-2861_st | 15.7733304 | 0.353919206 |
| hsa-miR-31_st | 8.33047045 | 3.06654134 |
| hsa-miR-367_st | 2.913663117 | 0.445397956 |
| hsa-miR-548c-3p_st | 2.551935828 | 0.556166374 |
| hsa-miR-548c-5p_st | 5.080646261 | 0.831691727 |
| hsa-miR-548d-3p_st | 3.356206857 | 0.328192312 |
| hsa-miR-548d-5p_st | 4.559829267 | 0.663743403 |
| hsa-miR-548e_st | 3.022477778 | 0.198223027 |
| hsa-miR-548f_st | 3.292803315 | 0.25201269 |
| hsa-miR-548g_st | 2.890931986 | 0.439552409 |
| hsa-miR-548h_st | 3.924006372 | 0.505485067 |
| hsa-miR-548i_st | 4.167615025 | 0.393444005 |
| hsa-miR-548j_st | 4.439604377 | 0.641083348 |
| hsa-miR-548k_st | 3.620233304 | 0.196363404 |
| hsa-miR-526b-star_st | 2.9672203 | 0.308900456 |
| hsa-miR-548m_st | 3.892823079 | 0.306192177 |
| hsa-miR-548n_st | 3.406112194 | 0.230134883 |
| hsa-miR-548o_st | 2.770759687 | 0.310507008 |
| hsa-miR-548p_st | 2.942716174 | 0.484313272 |
| hsa-miR-548q_st | 9.518485506 | 0.550786137 |
| hsa-miR-548s_st | 3.237843445 | 0.365318126 |
| hsa-miR-548t_st | 3.781180016 | 0.3890071 |
| hsa-miR-548u_st | 2.92668201 | 0.559177899 |
| hsa-miR-638_st | 15.80450035 | 0.414582408 |
| hsa-miR-548w_st | 4.576024101 | 0.924545672 |
| hsa-miR-3119_st | 2.713499091 | 0.471134521 |
| hsa-miR-549_st | 3.691073859 | 0.321937249 |
| hsa-miR-205_st | 6.197855385 | 2.918864929 |
| hsa-miR-550-star_st | 5.356351262 | 1.020352829 |
| hsa-miR-551a_st | 4.361039914 | 0.558662414 |
| hsa-miR-93_st | 10.46192701 | 0.768751285 |
| hsa-miR-551b-star_st | 11.5951683 | 0.489391408 |
| hsa-let-7d-star_st | 8.292860977 | 1.463444009 |
| hsa-miR-553_st | 3.485148328 | 0.370999863 |
| hsa-miR-554_st | 4.065280733 | 0.235432902 |
| hsa-miR-555_st | 3.97615877 | 0.350079823 |
| hsa-miR-556-3p_st | 3.231187659 | 0.362064433 |
| hsa-miR-556-5p_st | 3.07323592 | 0.315889203 |
| hsa-miR-557_st | 11.99752118 | 0.437539131 |
| hsa-miR-558_st | 3.447643248 | 0.355422175 |
| hsa-miR-559_st | 3.32875335 | 0.329172039 |
| hsa-miR-561_st | 3.372329946 | 0.383286749 |
| hsa-miR-562_st | 3.32007404 | 0.308791932 |
| hsa-miR-563_st | 4.001658318 | 0.421946818 |
| hsa-miR-361-5p_st | 11.47227742 | 0.795437576 |
| hsa-miR-566_st | 6.617457293 | 0.273738481 |
| hsa-miR-567_st | 3.803856367 | 0.742263077 |
| hsa-miR-568_st | 3.046832404 | 0.358362772 |
| hsa-miR-29c_st | 12.58141857 | 0.732046128 |
| hsa-miR-570_st | 3.254665253 | 0.21364129 |
| hsa-miR-571_st | 4.153734266 | 0.467807748 |
| hsa-miR-572_st | 14.23434413 | 0.25506185 |
| hsa-miR-573_st | 3.364949517 | 0.269180224 |
| hsa-miR-132_st | 7.495199671 | 1.611518447 |
| hsa-miR-574-5p_st | 8.191959706 | 0.409517456 |
| hsa-let-7d_st | 14.44259776 | 0.486427429 |
| hsa-miR-576-3p_st | 4.691598101 | 0.539145273 |
| hsa-miR-576-5p_st | 4.045272561 | 0.621427033 |
| hsa-miR-577_st | 3.215188096 | 0.458326752 |
| hsa-miR-578_st | 4.175572717 | 0.310672743 |
| hsa-miR-579_st | 3.355939711 | 0.296231116 |
| hsa-miR-580_st | 3.177991807 | 0.31242122 |
| hsa-miR-191_st | 12.8294704 | 0.745669275 |
| hsa-miR-582-3p_st | 3.891236637 | 0.247609148 |
| hsa-miR-582-5p_st | 3.48072578 | 0.993854804 |
| hsa-miR-1915_st | 15.80445781 | 0.41458079 |
| hsa-let-7c_st | 14.33965474 | 0.56142438 |
| hsa-miR-585_st | 3.676706595 | 0.325242062 |
| hsa-miR-586_st | 3.115435748 | 0.330633826 |
| hsa-miR-324-5p_st | 9.334038374 | 0.889638423 |
| hsa-miR-588_st | 5.189310898 | 0.4139754 |
| hsa-miR-589_st | 5.045287429 | 0.464812176 |
| hsa-miR-589-star_st | 5.168989742 | 0.485859056 |
| hsa-miR-188-5p_st | 8.4050448 | 0.611669083 |
| hsa-miR-103_st | 13.09210474 | 0.669298536 |
| hsa-miR-591_st | 4.037498622 | 0.436401955 |
| hsa-miR-592_st | 3.480573401 | 0.662663833 |
| hsa-miR-593_st | 6.042201039 | 0.333899469 |
| hsa-miR-23b_st | 14.57717778 | 0.57446395 |
| hsa-miR-595_st | 7.02794467 | 0.335397875 |
| hsa-miR-99a_st | 12.61401811 | 1.232242591 |
| hsa-miR-15b_st | 10.90719793 | 1.257104115 |
| hsa-miR-135b_st | 4.372410287 | 0.939107468 |
| hsa-miR-599_st | 3.058379929 | 0.519899617 |
| hsa-miR-600_st | 7.072488005 | 0.637333381 |
| hsa-miR-107_st | 13.00670539 | 0.626160471 |
| hsa-miR-602_st | 14.05079982 | 0.282034361 |
| hsa-miR-603_st | 2.98214102 | 0.256461477 |
| hsa-miR-3065-5p_st | 4.563223362 | 1.440582635 |
| hsa-miR-605_st | 5.286786379 | 0.483434825 |
| hsa-miR-24_st | 13.50428429 | 0.663999897 |
| hsa-miR-607_st | 3.448105584 | 0.247464955 |
| hsa-miR-608_st | 9.435447562 | 0.259115932 |
| hsa-miR-609_st | 3.783826276 | 0.312615116 |
| hsa-miR-3196_st | 15.80552392 | 0.415737295 |
| hsa-miR-26b_st | 13.06592106 | 0.688443983 |
| hsa-miR-28-5p_st | 10.02122476 | 0.489418533 |
| hsa-miR-613_st | 4.030864406 | 0.294413709 |
| hsa-miR-338-3p_st | 5.325298242 | 1.493243358 |
| hsa-miR-615-3p_st | 7.380334768 | 0.463290678 |
| hsa-miR-454_st | 7.163103195 | 0.858902895 |
| hsa-miR-616_st | 8.053792417 | 0.511570269 |
| hsa-miR-151-5p_st | 12.28749225 | 0.463419016 |
| hsa-miR-617_st | 7.159992296 | 0.873521986 |
| hsa-miR-128_st | 9.351785733 | 1.053477289 |
| hsa-miR-619_st | 4.486472078 | 0.366900595 |
| hsa-miR-620_st | 3.64624706 | 0.300151884 |
| hsa-miR-621_st | 4.016026495 | 0.382657472 |
| hsa-let-7b_st | 13.31874955 | 1.009507341 |
| hsa-miR-623_st | 9.873685431 | 0.216815663 |
| hsa-miR-624_st | 4.01181992 | 0.285100232 |
| hsa-miR-624-star_st | 4.332323405 | 0.72405747 |
| hsa-miR-183_st | 7.731525357 | 1.296542305 |
| hsa-miR-30b_st | 13.32721506 | 0.373900135 |
| hsa-miR-626_st | 3.293919864 | 0.372581198 |
| hsa-miR-627_st | 3.304561749 | 0.441759495 |
| hsa-miR-628-3p_st | 10.99900052 | 1.292811213 |
| hsa-miR-31-star_st | 4.468161718 | 3.069041371 |
| hsa-miR-218-1-star_st | 3.406688503 | 0.403448151 |
| hsa-miR-629-star_st | 6.135671925 | 0.516401266 |
| hsa-miR-630_st | 8.367283405 | 0.872774703 |
| hsa-miR-631_st | 7.292503851 | 1.004234432 |
| hsa-miR-632_st | 4.466774854 | 0.576344616 |
| hsa-miR-633_st | 3.189727739 | 0.328839765 |
| hsa-miR-634_st | 5.785665723 | 0.972531844 |
| hsa-miR-635_st | 5.061945763 | 0.613134614 |
| hsa-miR-203_st | 8.134155576 | 2.061222705 |
| hsa-miR-331-3p_st | 10.5128864 | 0.699834644 |
| hsa-miR-125b_st | 14.54584858 | 0.60575746 |
| hsa-let-7i_st | 13.62034015 | 0.724742139 |
| hsa-miR-640_st | 5.807272353 | 0.312804794 |
| hsa-miR-641_st | 4.84236123 | 0.6273217 |
| hsa-miR-642_st | 4.005526941 | 0.34892113 |
| hsa-miR-29b_st | 12.78590779 | 0.922218066 |
| hsa-miR-122_st | 4.401655648 | 0.433064488 |
| hsa-miR-645_st | 4.528626248 | 0.244132105 |
| hsa-miR-646_st | 3.982571016 | 0.442295364 |
| hsa-miR-647_st | 4.796297255 | 0.303511173 |
| hsa-miR-648_st | 8.681869631 | 0.393242871 |
| hsa-miR-649_st | 4.021342918 | 0.701149587 |
| hsa-miR-182_st | 10.58249999 | 1.08710128 |
| hsa-miR-651_st | 3.129403256 | 0.352182192 |
| hsa-miR-652_st | 7.877508626 | 0.768591455 |
| hsa-miR-653_st | 3.070031603 | 0.300380206 |
| hsa-miR-654-3p_st | 4.719034401 | 1.653691056 |
| hsa-miR-374b_st | 8.311444342 | 0.685749139 |
| hsa-miR-130a_st | 11.69154937 | 0.686214041 |
| hsa-miR-656_st | 3.578491785 | 0.342033843 |
| hsa-miR-657_st | 4.164262545 | 0.436292898 |
| hsa-miR-658_st | 12.09008412 | 0.399339252 |
| hsa-miR-99b_st | 11.72051 | 0.810486917 |
| hsa-miR-660_st | 8.42891977 | 1.067247336 |
| hsa-miR-661_st | 7.090628412 | 0.376992477 |
| hsa-miR-30e-star_st | 10.47292099 | 1.143237493 |
| hsa-miR-663_st | 15.60499297 | 0.358699166 |
| hsa-miR-663b_st | 10.23191101 | 0.413480867 |
| hsa-miR-664_st | 7.889870823 | 0.787406697 |
| hsa-miR-664-star_st | 10.11457456 | 0.828182343 |
| hsa-miR-30d_st | 13.04724431 | 0.466532488 |
| hsa-miR-668_st | 8.571288609 | 0.575571482 |
| hsa-miR-670_st | 5.160797226 | 0.554286761 |
| hsa-miR-671-3p_st | 7.499483176 | 0.524564387 |
| hsa-miR-671-5p_st | 12.25676283 | 0.232774365 |
| hsa-miR-30a-star_st | 10.82252443 | 1.154206027 |
| hsa-miR-675-star_st | 5.112394025 | 0.520294656 |
| hsa-miR-148b_st | 9.93977646 | 0.771471228 |
| hsa-miR-708_st | 7.940079113 | 1.564941682 |
| hsa-miR-708-star_st | 4.296320901 | 0.353886218 |
| hsa-miR-221-star_st | 6.924257339 | 1.686940568 |
| hsa-let-7e_st | 13.14530741 | 0.696323234 |
| hsa-miR-7-1-star_st | 6.585159303 | 0.856104437 |
| hsa-miR-27b_st | 12.98670716 | 0.447334038 |
| hsa-miR-34a_st | 9.752045866 | 1.354852932 |
| hsa-miR-744_st | 14.33236852 | 0.381917587 |
| hsa-miR-744-star_st | 5.186310427 | 0.441038113 |
| hsa-miR-92b_st | 9.616842204 | 1.445623964 |
| hsa-miR-759_st | 3.293874756 | 0.421380174 |
| hsa-miR-760_st | 10.29890681 | 0.492749846 |
| hsa-miR-92a_st | 11.61845971 | 1.194701029 |
| hsa-miR-335_st | 9.99805058 | 0.847187434 |
| hsa-miR-96_st | 8.153083944 | 1.206737605 |
| hsa-miR-95_st | 8.788939753 | 1.007518409 |
| hsa-miR-766_st | 7.538910686 | 0.345905123 |
| hsa-miR-767-3p_st | 4.202930904 | 0.226676867 |
| hsa-miR-767-5p_st | 4.36764344 | 0.449657305 |
| hsa-miR-98_st | 10.65074756 | 0.808305776 |
| hsa-miR-769-5p_st | 7.394684018 | 0.807342748 |
| hsa-miR-770-5p_st | 7.799338826 | 0.544174665 |
| hsa-miR-802_st | 2.910978116 | 0.287417396 |
| hsa-miR-34b-star_st | 6.452815734 | 1.229679145 |
| hsa-miR-581_st | 2.69005347 | 0.622170491 |
| hsa-miR-181d_st | 9.497680111 | 0.802187112 |
| hsa-miR-875-5p_st | 2.866422213 | 0.352115979 |
| hsa-miR-876-3p_st | 3.22502718 | 0.35181295 |
| hsa-miR-876-5p_st | 2.636975274 | 0.407181072 |
| hsa-miR-151-3p_st | 9.928450931 | 0.511242956 |
| hsa-miR-877-star_st | 5.985272902 | 0.662343654 |
| hsa-miR-885-3p_st | 13.33552778 | 0.521981099 |
| hsa-miR-200c_st | 13.89146666 | 0.446453296 |
| hsa-miR-429_st | 5.835742762 | 1.814180085 |
| hsa-miR-886-5p_st | 12.83676585 | 0.879179868 |
| hsa-miR-224_st | 8.047711461 | 1.374030683 |
| hsa-miR-888_st | 3.480056641 | 0.570579776 |
| hsa-miR-4321_st | 9.605216576 | 0.455188582 |
| hsa-miR-889_st | 3.416494703 | 0.402710511 |
| hsa-miR-181b_st | 10.7791005 | 0.792412486 |
| hsa-miR-891a_st | 6.959925918 | 0.629228746 |
| hsa-miR-891b_st | 3.941669022 | 0.488002907 |
| hsa-miR-892a_st | 4.368166152 | 0.842683271 |
| hsa-miR-892b_st | 4.64473837 | 0.447772897 |
| hsa-miR-9_st | 2.97576649 | 0.324639663 |
| hsa-miR-222_st | 12.34217148 | 1.918798705 |
| hsa-miR-141_st | 10.47024842 | 0.725536346 |
| hsa-miR-221_st | 12.56379474 | 1.641533028 |
| hsa-miR-924_st | 3.899667356 | 0.229009897 |
| hsa-miR-181a_st | 12.46367044 | 1.097214185 |
| hsa-miR-181c_st | 9.310571105 | 1.052647573 |
| hsa-miR-4324_st | 10.65482238 | 1.178890301 |
| hsa-miR-125a-5p_st | 13.96720564 | 1.047507597 |
| hsa-miR-92b-star_st | 14.67935209 | 0.264384316 |
| hsa-miR-3195_st | 12.69957006 | 0.4431018 |
| hsa-miR-3137_st | 10.94948284 | 0.589448822 |
| hsa-miR-934_st | 5.316019706 | 0.606167733 |
| hsa-miR-935_st | 4.766481857 | 0.744618673 |
| hsa-miR-551b_st | 4.959840516 | 2.498427895 |
| hsa-miR-937_st | 5.441795028 | 0.760816242 |
| hsa-miR-938_st | 4.901024337 | 0.402229406 |
| hsa-miR-939_st | 13.19242244 | 0.278406151 |
| hsa-miR-7-2-star_st | 6.461874071 | 1.823716389 |
| hsa-miR-146b-5p_st | 10.72460716 | 2.254121832 |
| hsa-miR-941_st | 5.8472898 | 0.527232251 |
| hsa-miR-942_st | 3.132337606 | 0.448407639 |
| hsa-miR-200a_st | 9.063667443 | 2.478819937 |
| hsa-miR-944_st | 3.475952551 | 0.505791777 |
| hsa-miR-7_st | 7.569002947 | 3.423226794 |
| hsa-miR-375_st | 5.973873271 | 0.890249857 |
| hsa-miR-96-star_st | 4.54741107 | 0.550003676 |
| hsa-miR-424_st | 9.200903135 | 1.485182236 |
| hsa-miR-181a-2-star_st | 8.649746134 | 1.060700412 |
| hsa-miR-200b_st | 10.36920802 | 2.416748349 |
| hsa-miR-146b-3p_st | 5.411185853 | 1.972804791 |
| hsa-miR-99b-star_st | 7.752210153 | 0.491251051 |
| hsa-miR-9-star_st | 4.077600376 | 0.578675413 |

| | Log2 Diff | Fold change | T-test | |
|---|---|---|---|---|
| | (Malignant vs | (Malignant vs | (Malignant vs | |
| Sample Name | Benign) | Benign) | Benign) | p < 0.05 |
| hsa-miR-150_st | 0.222052605 | 1.2 | 0.860205599 | 0 |
| hsa-let-7a-2-star_st | 0.380521359 | 1.3 | 0.021585956 | 1 |
| hsa-let-7a-star_st | 0.173921871 | 1.1 | 0.485965006 | 0 |
| hsa-miR-513b_st | −0.942170264 | 1.9 | 0.035004076 | 1 |
| hsa-let-7b-star_st | 0.303508552 | 1.2 | 0.052070371 | 0 |
| hsa-miR-593-star_st | −0.844203562 | 1.8 | 0.042498824 | 1 |
| hsa-let-7c-star_st | 0.160682635 | 1.1 | 0.242588862 | 0 |
| hsa-miR-516a-5p_st | −0.7087716 | 1.6 | 0.019004175 | 1 |
| hsa-miR-142-5p_st | 0.864378114 | 1.8 | 0.208465715 | 0 |
| hsa-miR-650_st | −0.659154321 | 1.6 | 0.003369901 | 1 |
| hsa-miR-513c_st | −0.773527809 | 1.7 | 0.02662366 | 1 |
| hsa-miR-761_st | −0.436015764 | 1.4 | 0.282013089 | 0 |
| hsa-let-7f-1-star_st | −0.056873642 | 1.0 | 0.994754986 | 0 |
| hsa-miR-513a-5p_st | −0.710774544 | 1.6 | 0.055858062 | 0 |
| hsa-miR-654-5p_st | −0.874558595 | 1.8 | 0.002216159 | 1 |
| hsa-let-7g-star_st | 0.187885637 | 1.1 | 0.162150496 | 0 |
| hsa-miR-3163_st | −0.615485135 | 1.5 | 0.014703979 | 1 |
| hsa-let-7i-star_st | 0.975051153 | 2.0 | 9.84706E−05 | 1 |
| hsa-miR-3165_st | −0.716115742 | 1.6 | 0.024092301 | 1 |
| hsa-miR-3194_st | −0.71116551 | 1.6 | 0.002778842 | 1 |
| hsa-miR-100-star_st | 0.147806712 | 1.1 | 0.679726515 | 0 |
| hsa-miR-4251_st | −0.555906938 | 1.5 | 0.046641914 | 1 |
| hsa-miR-101-star_st | 0.396387368 | 1.3 | 0.216028834 | 0 |
| hsa-miR-922_st | −0.383665861 | 1.3 | 0.120758822 | 0 |
| hsa-miR-103-2-star_st | 0.409014576 | 1.3 | 0.453881694 | 0 |
| hsa-miR-155_st | 0.657110316 | 1.6 | 0.068541709 | 0 |
| hsa-miR-105_st | −0.199067097 | 1.1 | 0.039953133 | 1 |
| hsa-miR-127-5p_st | −0.480881806 | 1.4 | 0.197776991 | 0 |
| hsa-miR-1286_st | −0.361311766 | 1.3 | 0.405657897 | 0 |
| hsa-miR-106a-star_st | 0.10908219 | 1.1 | 0.958945377 | 0 |
| hsa-miR-890_st | −0.175051975 | 1.1 | 0.617679044 | 0 |
| hsa-miR-3126-5p_st | −0.880040173 | 1.8 | 0.002258153 | 1 |
| hsa-miR-489_st | −0.695721922 | 1.6 | 0.038958849 | 1 |
| hsa-miR-10a_st | 0.893140031 | 1.9 | 0.007144861 | 1 |
| hsa-miR-10a-star_st | 0.034602902 | 1.0 | 0.704138675 | 0 |
| hsa-miR-10b_st | 1.236475278 | 2.4 | 0.000452127 | 1 |
| hsa-miR-10b-star_st | 0.239974108 | 1.2 | 0.134880405 | 0 |
| hsa-miR-1178_st | −0.22990198 | 1.2 | 0.144231856 | 0 |
| hsa-miR-1179_st | 0.179450609 | 1.1 | 0.260135043 | 0 |
| hsa-miR-1180_st | −0.3957419 | 1.3 | 0.001411079 | 1 |
| hsa-miR-1181_st | −0.289322804 | 1.2 | 0.140870772 | 0 |
| hsa-miR-3138_st | −0.792844548 | 1.7 | 0.001393198 | 1 |
| hsa-miR-220c_st | −0.46156797 | 1.4 | 0.043585179 | 1 |
| hsa-miR-510_st | −0.761174501 | 1.7 | 0.010677787 | 1 |
| hsa-miR-1185_st | −0.059965152 | 1.0 | 0.485006514 | 0 |
| hsa-miR-1193_st | −0.293261361 | 1.2 | 0.018262307 | 1 |
| hsa-miR-1197_st | 0.005404133 | 1.0 | 0.964332774 | 0 |
| hsa-miR-1200_st | 0.038508566 | 1.0 | 0.956740049 | 0 |
| hsa-miR-1201_st | 0.536104713 | 1.5 | 0.102280969 | 0 |
| hsa-miR-1202_st | −0.579908533 | 1.5 | 0.000136312 | 1 |
| hsa-miR-30c-1-star_st | −0.683488343 | 1.6 | 0.015959194 | 1 |
| hsa-miR-1204_st | −0.234005785 | 1.2 | 0.196014043 | 0 |
| hsa-miR-1205_st | −0.159132582 | 1.1 | 0.154185041 | 0 |
| hsa-miR-1206_st | 0.027885919 | 1.0 | 0.959626916 | 0 |
| hsa-miR-1207-3p_st | −0.109163301 | 1.1 | 0.34613346 | 0 |
| hsa-miR-1207-5p_st | −0.461218285 | 1.4 | 0.000299713 | 1 |
| hsa-miR-1208_st | −0.810294521 | 1.8 | 7.59262E−05 | 1 |
| hsa-miR-622_st | −0.540947195 | 1.5 | 0.078427604 | 0 |
| hsa-miR-1224-3p_st | −0.010074347 | 1.0 | 0.992062807 | 0 |
| hsa-miR-2116_st | −0.514250548 | 1.4 | 0.033503517 | 1 |
| hsa-miR-1225-3p_st | −0.172683387 | 1.1 | 0.547414109 | 0 |
| hsa-miR-1225-5p_st | −0.517668035 | 1.4 | 0.000801489 | 1 |
| hsa-miR-1226_st | −0.193009106 | 1.1 | 0.14831534 | 0 |
| hsa-miR-30b-star_st | −0.659411627 | 1.6 | 0.011099023 | 1 |
| hsa-miR-1227_st | 0.08972765 | 1.1 | 0.977701762 | 0 |
| hsa-miR-1295_st | −0.52190788 | 1.4 | 0.003859278 | 1 |
| hsa-miR-1228-star_st | −0.301796436 | 1.2 | 0.066221826 | 0 |
| hsa-miR-20b_st | 1.450315379 | 2.7 | 0.001914349 | 1 |
| hsa-miR-122-star_st | 0.033323905 | 1.0 | 0.637196006 | 0 |
| hsa-miR-1231_st | −0.539214969 | 1.5 | 0.000314227 | 1 |
| hsa-miR-1233_st | −0.368931947 | 1.3 | 0.033666673 | 1 |
| hsa-miR-524-5p_st | −0.194758584 | 1.1 | 0.442305294 | 0 |
| hsa-miR-1236_st | −0.030539642 | 1.0 | 0.847291014 | 0 |
| hsa-miR-873_st | −0.317384348 | 1.2 | 0.096861262 | 0 |
| hsa-miR-1238_st | −0.245755821 | 1.2 | 0.276479632 | 0 |
| hsa-miR-124_st | −0.149975326 | 1.1 | 0.410583674 | 0 |
| hsa-miR-1243_st | −0.007320981 | 1.0 | 0.940745356 | 0 |
| hsa-miR-1244_st | 0.120529402 | 1.1 | 0.320982615 | 0 |
| hsa-miR-1245_st | −0.072612339 | 1.1 | 0.459296555 | 0 |
| hsa-miR-3122_st | −0.838109462 | 1.8 | 0.000229025 | 1 |
| hsa-miR-134_st | −0.825685919 | 1.8 | 0.000221367 | 1 |
| hsa-miR-1248_st | −0.050695798 | 1.0 | 0.858578269 | 0 |
| hsa-miR-146a_st | 1.703691806 | 3.3 | 0.000239942 | 1 |
| hsa-miR-124-star_st | −0.243733207 | 1.2 | 0.060226873 | 0 |
| hsa-miR-1250_st | −0.607792994 | 1.5 | 0.003333519 | 1 |
| hsa-miR-764_st | −0.318323323 | 1.2 | 0.204880907 | 0 |
| hsa-miR-1252_st | 0.205286327 | 1.2 | 0.196771547 | 0 |
| hsa-miR-223_st | 0.973175877 | 2.0 | 0.065579732 | 0 |
| hsa-miR-34c-3p_st | −0.652186785 | 1.6 | 0.029729915 | 1 |
| hsa-miR-1255a_st | 0.131786979 | 1.1 | 0.817077716 | 0 |
| hsa-miR-1255b_st | −0.252264174 | 1.2 | 0.333691681 | 0 |
| hsa-miR-1256_st | 0.107340868 | 1.1 | 0.731503578 | 0 |
| hsa-miR-1257_st | 0.019477962 | 1.0 | 0.988403769 | 0 |
| hsa-miR-199b-5p_st | −0.21529813 | 1.2 | 0.774554017 | 0 |
| hsa-miR-1259_st | 0.182349168 | 1.1 | 0.52426746 | 0 |
| hsa-miR-492_st | −0.642006592 | 1.6 | 0.006480247 | 1 |
| hsa-miR-3132_st | −0.584728046 | 1.5 | 0.0386641 | 1 |
| hsa-miR-516b_st | −0.561909372 | 1.5 | 0.014122583 | 1 |
| hsa-miR-4294_st | −0.701213945 | 1.6 | 0.003054414 | 1 |
| hsa-miR-125b-2-star_st | −0.262893484 | 1.2 | 0.400570054 | 0 |
| hsa-miR-126_st | 0.506142921 | 1.4 | 0.068976141 | 0 |
| hsa-miR-198_st | −0.700349187 | 1.6 | 0.004579127 | 1 |
| hsa-miR-3191_st | −0.477486945 | 1.4 | 0.119742016 | 0 |
| hsa-miR-1261_st | −0.516847212 | 1.4 | 0.147890143 | 0 |
| hsa-miR-1262_st | −0.109808362 | 1.1 | 0.395853993 | 0 |
| hsa-miR-1263_st | 0.113544564 | 1.1 | 0.54420963 | 0 |
| hsa-miR-1264_st | 0.063831943 | 1.0 | 0.779008887 | 0 |
| hsa-miR-1265_st | 0.173811188 | 1.1 | 0.140514249 | 0 |
| hsa-miR-2114-star_st | −0.503348396 | 1.4 | 0.091566835 | 0 |
| hsa-miR-1267_st | −0.257862054 | 1.2 | 0.024872184 | 1 |
| hsa-miR-1268_st | −0.487502765 | 1.4 | 0.002166064 | 1 |
| hsa-miR-1269_st | −0.37033274 | 1.3 | 0.04866386 | 1 |
| hsa-miR-509-5p_st | −0.459679404 | 1.4 | 0.044420536 | 1 |
| hsa-miR-1270_st | −0.272207857 | 1.2 | 0.033363679 | 1 |
| hsa-miR-1271_st | −0.272891287 | 1.2 | 0.124529908 | 0 |
| hsa-miR-4303_st | −0.499112595 | 1.4 | 0.044577217 | 1 |
| hsa-miR-1273_st | −0.487899233 | 1.4 | 0.088223736 | 0 |
| hsa-miR-583_st | −0.237961517 | 1.2 | 0.405576105 | 0 |
| hsa-miR-1273d_st | −0.263304276 | 1.2 | 0.208253538 | 0 |
| hsa-miR-127-3p_st | 0.465906015 | 1.4 | 0.191036356 | 0 |
| hsa-miR-522-star_st | −0.56698063 | 1.5 | 0.021536929 | 1 |
| hsa-miR-214-star_st | 0.159707729 | 1.1 | 0.375439189 | 0 |
| hsa-miR-184_st | −0.554802057 | 1.5 | 0.092597264 | 0 |
| hsa-miR-614_st | −0.316560945 | 1.2 | 0.176367012 | 0 |
| hsa-miR-526b_st | −0.346183854 | 1.3 | 0.120643184 | 0 |
| hsa-miR-1277_st | 0.216959641 | 1.2 | 0.611342978 | 0 |
| hsa-miR-1278_st | 0.229200297 | 1.2 | 0.316218283 | 0 |
| hsa-miR-1279_st | 0.116000432 | 1.1 | 0.568339756 | 0 |
| hsa-miR-409-3p_st | −0.119256413 | 1.1 | 0.271379705 | 0 |
| hsa-miR-199a-5p_st | 0.266041662 | 1.2 | 0.368234862 | 0 |
| hsa-miR-1281_st | −0.376508269 | 1.3 | 0.015708011 | 1 |
| hsa-miR-1282_st | −0.07627604 | 1.1 | 0.635598922 | 0 |
| hsa-miR-33b-star_st | −0.536314174 | 1.5 | 0.003531689 | 1 |
| hsa-miR-1284_st | −0.043979139 | 1.0 | 0.453609027 | 0 |
| hsa-miR-1253_st | −0.149617083 | 1.1 | 0.40442235 | 0 |
| hsa-miR-584_st | −0.595345983 | 1.5 | 0.00107128 | 1 |
| hsa-miR-1287_st | −0.005456183 | 1.0 | 0.606367673 | 0 |
| hsa-miR-15a_st | 1.890662707 | 3.7 | 4.99062E−05 | 1 |
| hsa-miR-1289_st | −0.079816411 | 1.1 | 0.628434489 | 0 |
| hsa-miR-1290_st | 0.032634716 | 1.0 | 0.499675865 | 0 |
| hsa-miR-2117_st | −0.305112668 | 1.2 | 0.015940351 | 1 |
| hsa-miR-1292_st | −0.611743889 | 1.5 | 0.000848166 | 1 |
| hsa-miR-1293_st | −0.51026792 | 1.4 | 0.002466981 | 1 |
| hsa-miR-129-3p_st | 0.383345945 | 1.3 | 0.19794232 | 0 |
| hsa-miR-520d-5p_st | −0.412973182 | 1.3 | 0.119864002 | 0 |
| hsa-miR-526a_st | −0.298084351 | 1.2 | 0.258970121 | 0 |
| hsa-miR-129-5p_st | −0.66296401 | 1.6 | 0.000187048 | 1 |
| hsa-miR-1254_st | −0.701761114 | 1.6 | 0.000549994 | 1 |
| hsa-miR-1297_st | −0.012651212 | 1.0 | 0.596075545 | 0 |
| hsa-miR-1298_st | 0.055368649 | 1.0 | 0.670207362 | 0 |
| hsa-miR-1299_st | −0.150683798 | 1.1 | 0.456002363 | 0 |
| hsa-miR-129-star_st | 0.136663049 | 1.1 | 0.836867788 | 0 |
| hsa-miR-1301_st | −0.301427895 | 1.2 | 0.017730122 | 1 |
| hsa-miR-19b_st | 1.38730385 | 2.6 | 0.002656394 | 1 |
| hsa-miR-1303_st | −0.404276523 | 1.3 | 0.057547753 | 0 |
| hsa-miR-1304_st | −0.312528984 | 1.2 | 0.141834548 | 0 |
| hsa-miR-1305_st | −0.063087622 | 1.0 | 0.378970808 | 0 |
| hsa-miR-550_st | −0.496093635 | 1.4 | 0.007154735 | 1 |
| hsa-miR-1307_st | −0.554199521 | 1.5 | 0.001022818 | 1 |
| hsa-miR-1308_st | 0.310171917 | 1.2 | 0.021134089 | 1 |
| hsa-miR-4291_st | 0.769804555 | 1.7 | 0.002661911 | 1 |
| hsa-miR-130a-star_st | 0.006223946 | 1.0 | 0.589744809 | 0 |
| hsa-miR-130b_st | 0.417136587 | 1.3 | 0.271794998 | 0 |
| hsa-miR-130b-star_st | 0.275304414 | 1.2 | 0.422166352 | 0 |
| hsa-miR-921_st | −0.671560214 | 1.6 | 0.004421654 | 1 |
| hsa-miR-520a-5p_st | −0.264381023 | 1.2 | 0.07023584 | 0 |
| hsa-miR-711_st | −0.565600546 | 1.5 | 0.01609509 | 1 |
| hsa-miR-1323_st | −0.640389031 | 1.6 | 0.00808267 | 1 |
| hsa-miR-1324_st | 0.135696574 | 1.1 | 0.548358212 | 0 |
| hsa-miR-490-3p_st | −0.435769493 | 1.4 | 0.056015659 | 0 |
| hsa-miR-718_st | −0.599517099 | 1.5 | 0.004795659 | 1 |
| hsa-miR-133b_st | 0.410552029 | 1.3 | 0.236027744 | 0 |
| hsa-miR-431-star_st | −0.689803635 | 1.6 | 0.000147175 | 1 |
| hsa-miR-552_st | −0.449931627 | 1.4 | 0.060962583 | 0 |
| hsa-miR-28-3p_st | 0.794466886 | 1.7 | 0.009148888 | 1 |
| hsa-miR-4265_st | −0.711203667 | 1.6 | 0.000455925 | 1 |
| hsa-miR-643_st | −0.262740454 | 1.2 | 0.015336244 | 1 |
| hsa-miR-136_st | 0.142073896 | 1.1 | 0.624399868 | 0 |
| hsa-miR-136-star_st | 0.103574111 | 1.1 | 0.805234671 | 0 |
| hsa-miR-137_st | 0.049307813 | 1.0 | 0.94879969 | 0 |
| hsa-miR-138_st | −0.238711587 | 1.2 | 0.935750036 | 0 |
| hsa-miR-138-1-star_st | −0.437943957 | 1.4 | 0.326726236 | 0 |
| hsa-miR-138-2-star_st | 0.083980864 | 1.1 | 0.490695804 | 0 |
| hsa-miR-139-3p_st | −0.370786241 | 1.3 | 0.106662544 | 0 |
| hsa-miR-139-5p_st | −0.091346369 | 1.1 | 0.977828318 | 0 |
| hsa-miR-140-3p_st | 0.338868465 | 1.3 | 0.29627309 | 0 |
| hsa-miR-140-5p_st | 0.972300809 | 2.0 | 0.009051354 | 1 |
| hsa-miR-381_st | −0.263385689 | 1.2 | 0.414203152 | 0 |
| hsa-miR-141-star_st | 0.540661697 | 1.5 | 0.00314116 | 1 |
| hsa-miR-17-star_st | 0.949839154 | 1.9 | 0.003725931 | 1 |
| hsa-miR-4300_st | −0.492348072 | 1.4 | 0.095278637 | 0 |
| hsa-miR-143_st | 0.518722575 | 1.4 | 0.024609474 | 1 |
| hsa-miR-143-star_st | 0.565456794 | 1.5 | 0.003663467 | 1 |
| hsa-miR-144_st | −0.028892495 | 1.0 | 0.285478952 | 0 |
| hsa-miR-3198_st | −0.610641835 | 1.5 | 0.002516015 | 1 |
| hsa-miR-145_st | 0.249698607 | 1.2 | 0.173696217 | 0 |
| hsa-miR-145-star_st | 0.522747956 | 1.4 | 0.047488588 | 1 |
| hsa-miR-1468_st | −0.07967708 | 1.1 | 0.918650785 | 0 |
| hsa-miR-1469_st | −0.098271541 | 1.1 | 0.53146079 | 0 |
| hsa-miR-1973_st | −0.618155465 | 1.5 | 0.029852895 | 1 |
| hsa-miR-3154_st | −0.610121669 | 1.5 | 0.001421936 | 1 |
| hsa-miR-148a-star_st | 0.152713606 | 1.1 | 0.555555741 | 0 |
| hsa-miR-92a-1-star_st | −0.626094587 | 1.5 | 0.001472805 | 1 |
| hsa-miR-147_st | −0.1478104 | 1.1 | 0.22943945 | 0 |
| hsa-miR-194-star_st | −0.635308948 | 1.6 | 0.014306649 | 1 |
| hsa-miR-1471_st | −0.765524331 | 1.7 | 0.00263111 | 1 |
| hsa-miR-147b_st | −0.049319243 | 1.0 | 0.766943913 | 0 |
| hsa-miR-148a_st | 0.47072386 | 1.4 | 0.185956664 | 0 |
| hsa-miR-4299_st | −0.583862034 | 1.5 | 0.052157931 | 0 |
| hsa-miR-135a-star_st | −0.682970357 | 1.6 | 0.001086185 | 1 |
| hsa-miR-612_st | −0.641610843 | 1.6 | 0.004947708 | 1 |
| hsa-miR-1321_st | −0.496037076 | 1.4 | 0.064938111 | 0 |
| hsa-miR-149-star_st | −0.243117708 | 1.2 | 0.10108445 | 0 |
| hsa-miR-604_st | −0.423099533 | 1.3 | 0.127502863 | 0 |
| hsa-miR-150-star_st | −0.520200751 | 1.4 | 0.001726856 | 1 |
| hsa-miR-125b-1-star_st | −0.52408129 | 1.4 | 0.001997452 | 1 |
| hsa-miR-1183_st | −0.548470484 | 1.5 | 0.002313088 | 1 |
| hsa-miR-152_st | 0.212617324 | 1.2 | 0.265995123 | 0 |
| hsa-miR-153_st | 0.14649857 | 1.1 | 0.518892811 | 0 |
| hsa-miR-1537_st | 0.221604548 | 1.2 | 0.235352722 | 0 |
| hsa-miR-1538_st | −0.305081411 | 1.2 | 0.110134976 | 0 |
| hsa-miR-1539_st | −0.051582527 | 1.0 | 0.69981064 | 0 |
| hsa-miR-154_st | −0.111549184 | 1.1 | 0.280684098 | 0 |
| hsa-miR-154-star_st | −0.205709255 | 1.2 | 0.224200718 | 0 |
| hsa-miR-432-star_st | −0.090365483 | 1.1 | 0.513894695 | 0 |
| hsa-miR-155-star_st | 0.045786866 | 1.0 | 0.932315595 | 0 |
| hsa-miR-298_st | −0.55733809 | 1.5 | 0.018468242 | 1 |
| hsa-miR-15a-star_st | −0.375557567 | 1.3 | 0.009283093 | 1 |
| hsa-miR-2110_st | −0.570307953 | 1.5 | 0.002057111 | 1 |
| hsa-miR-15b-star_st | −0.035800443 | 1.0 | 0.313629061 | 0 |
| hsa-miR-1266_st | −0.65613913 | 1.6 | 0.000215871 | 1 |
| hsa-miR-16-1-star_st | 0.164148859 | 1.1 | 0.301652611 | 0 |
| hsa-miR-16-2-star_st | 0.374808099 | 1.3 | 0.333336508 | 0 |
| hsa-miR-490-5p_st | −0.528264182 | 1.4 | 0.023155835 | 1 |
| hsa-miR-19a_st | 1.323355992 | 2.5 | 0.015279098 | 1 |
| hsa-miR-3192_st | −0.611262185 | 1.5 | 0.012872496 | 1 |
| hsa-miR-3155_st | −0.358769919 | 1.3 | 0.052632477 | 0 |
| hsa-miR-187-star_st | −0.790439088 | 1.7 | 1.72368E−06 | 1 |
| hsa-miR-16_st | 1.187434133 | 2.3 | 4.99589E−05 | 1 |
| hsa-miR-936_st | −0.625089817 | 1.5 | 3.63119E−05 | 1 |
| hsa-miR-181c-star_st | −0.150304512 | 1.1 | 0.466389322 | 0 |
| hsa-miR-379_st | −0.07801705 | 1.1 | 0.525193243 | 0 |
| hsa-miR-370_st | −0.699186518 | 1.6 | 0.000183243 | 1 |
| hsa-miR-541_st | −0.539167375 | 1.5 | 0.04249099 | 1 |
| hsa-miR-518c-star_st | −0.562116062 | 1.5 | 0.034552014 | 1 |
| hsa-miR-487b_st | 1.389168874 | 2.6 | 2.34956E−07 | 1 |
| hsa-miR-182-star_st | 0.165626251 | 1.1 | 0.331102696 | 0 |
| hsa-miR-3121_st | −0.353628739 | 1.3 | 0.34314123 | 0 |
| hsa-miR-3190-5p_st | −0.637945995 | 1.6 | 0.00219839 | 1 |
| hsa-miR-1203_st | −0.576687389 | 1.5 | 0.001914925 | 1 |
| hsa-miR-185_st | 0.54002987 | 1.5 | 0.156622986 | 0 |
| hsa-miR-185-star_st | −0.561635079 | 1.5 | 0.00439566 | 1 |
| hsa-miR-4317_st | −0.425489077 | 1.3 | 0.055858283 | 0 |
| hsa-miR-186-star_st | 0.013949729 | 1.0 | 0.568387294 | 0 |
| hsa-miR-187_st | 0.277486327 | 1.2 | 0.13063065 | 0 |
| hsa-miR-4257_st | −0.649391272 | 1.6 | 0.001137341 | 1 |
| hsa-miR-188-3p_st | 0.158044582 | 1.1 | 0.208869727 | 0 |
| hsa-miR-1184_st | −0.093702185 | 1.1 | 0.523736249 | 0 |
| hsa-miR-18a_st | 0.695434238 | 1.6 | 0.278369133 | 0 |
| hsa-miR-18a-star_st | −0.052263971 | 1.0 | 0.334453803 | 0 |
| hsa-miR-3140_st | −0.17103481 | 1.1 | 0.129229934 | 0 |
| hsa-miR-18b-star_st | −0.060274491 | 1.0 | 0.275886344 | 0 |
| hsa-miR-190_st | −0.092140883 | 1.1 | 0.440823238 | 0 |
| hsa-miR-601_st | −0.56492771 | 1.5 | 0.003124604 | 1 |
| hsa-miR-1909_st | −0.388023024 | 1.3 | 0.017882341 | 1 |
| hsa-miR-342-5p_st | 0.510166643 | 1.4 | 0.160942342 | 0 |
| hsa-miR-575_st | −0.65919221 | 1.6 | 0.000648499 | 1 |
| hsa-miR-885-5p_st | −0.452974123 | 1.4 | 0.058979047 | 0 |
| hsa-miR-300_st | −0.369829745 | 1.3 | 0.175354401 | 0 |
| hsa-miR-1911_st | −0.080800566 | 1.1 | 0.58531652 | 0 |
| hsa-miR-1911-star_st | −0.027799097 | 1.0 | 0.983135145 | 0 |
| hsa-miR-1912_st | −0.008034504 | 1.0 | 0.849438024 | 0 |
| hsa-miR-4266_st | −0.497439833 | 1.4 | 0.009954683 | 1 |
| hsa-miR-1914_st | 0.056854562 | 1.0 | 0.541624919 | 0 |
| hsa-miR-3190-3p_st | −0.709319801 | 1.6 | 0.000860913 | 1 |
| hsa-miR-618_st | 0.041708331 | 1.0 | 0.8315395 | 0 |
| hsa-miR-1276_st | −0.677451675 | 1.6 | 0.0034958 | 1 |
| hsa-miR-191-star_st | −0.222265208 | 1.2 | 0.303984173 | 0 |
| hsa-miR-1258_st | −0.347996547 | 1.3 | 0.043288493 | 1 |
| hsa-miR-192-star_st | 0.234446033 | 1.2 | 0.732297956 | 0 |
| hsa-miR-193a-3p_st | 0.30576614 | 1.2 | 0.189573355 | 0 |
| hsa-miR-193a-5p_st | −0.592120768 | 1.5 | 0.000133336 | 1 |
| hsa-miR-3157_st | −0.400939077 | 1.3 | 0.000167806 | 1 |
| hsa-miR-193b-star_st | −0.483099108 | 1.4 | 0.010249798 | 1 |
| hsa-miR-194_st | 1.490688147 | 2.8 | 0.003457032 | 1 |
| hsa-miR-4307_st | −0.043618789 | 1.0 | 0.698059565 | 0 |
| hsa-miR-195_st | 0.311579531 | 1.2 | 0.391586559 | 0 |
| hsa-miR-610_st | −0.225288073 | 1.2 | 0.075442265 | 0 |
| hsa-miR-196a_st | 0.077940014 | 1.1 | 0.794099419 | 0 |
| hsa-miR-196a-star_st | −0.389591825 | 1.3 | 0.136676225 | 0 |
| hsa-miR-196b_st | −0.05527541 | 1.0 | 0.213253638 | 0 |
| hsa-miR-196b-star_st | −0.173508489 | 1.1 | 0.376939991 | 0 |
| hsa-let-7f-2-star_st | 0.149517823 | 1.1 | 0.35825894 | 0 |
| hsa-miR-1972_st | −0.333437545 | 1.3 | 0.071033796 | 0 |
| hsa-miR-3184_st | −0.60260069 | 1.5 | 0.004941904 | 1 |
| hsa-miR-523-star_st | −0.464857041 | 1.4 | 0.094665624 | 0 |
| hsa-miR-1976_st | −0.222551222 | 1.2 | 0.142978389 | 0 |
| hsa-miR-625-star_st | 0.219133854 | 1.2 | 0.37662704 | 0 |
| hsa-miR-587_st | −0.391083957 | 1.3 | 0.003825484 | 1 |
| hsa-miR-199a-3p_st | 0.119490334 | 1.1 | 0.653815561 | 0 |
| hsa-miR-1910_st | −0.454789479 | 1.4 | 0.00645943 | 1 |
| hsa-miR-199b-3p_st | 0.116752945 | 1.1 | 0.618217432 | 0 |
| hsa-miR-639_st | −0.624796364 | 1.5 | 0.001615785 | 1 |
| hsa-miR-1909-star_st | −0.214253172 | 1.2 | 0.212847481 | 0 |
| hsa-miR-19a-star_st | 0.011234424 | 1.0 | 0.687240996 | 0 |
| hsa-miR-29c-star_st | 1.108321756 | 2.2 | 0.000152931 | 1 |
| hsa-miR-940_st | −0.53305217 | 1.4 | 0.004700134 | 1 |
| hsa-miR-19b-2-star_st | −0.061734427 | 1.0 | 0.448702321 | 0 |
| hsa-miR-4269_st | −0.453807676 | 1.4 | 0.064105056 | 0 |
| hsa-miR-200a-star_st | 0.268742079 | 1.2 | 0.097169899 | 0 |
| hsa-miR-1182_st | −0.641650097 | 1.6 | 0.000304427 | 1 |
| hsa-miR-518f-star_st | −0.233621327 | 1.2 | 0.451251355 | 0 |
| hsa-miR-92a-2-star_st | −0.515299774 | 1.4 | 0.081651463 | 0 |
| hsa-miR-200c-star_st | 0.208084422 | 1.2 | 0.108188541 | 0 |
| hsa-miR-202_st | −0.491667701 | 1.4 | 0.006502044 | 1 |
| hsa-miR-202-star_st | 0.098346294 | 1.1 | 0.677482857 | 0 |
| hsa-miR-1914-star_st | −0.555736279 | 1.5 | 0.001401361 | 1 |
| hsa-miR-204_st | −1.399312568 | 2.6 | 0.051996525 | 0 |
| hsa-miR-665_st | −0.442075084 | 1.4 | 0.011902631 | 1 |
| hsa-miR-2052_st | 0.208547023 | 1.2 | 0.351417808 | 0 |
| hsa-miR-2053_st | 0.072093581 | 1.1 | 0.738327491 | 0 |
| hsa-miR-2054_st | 0.027790542 | 1.0 | 0.878952903 | 0 |
| hsa-miR-205-star_st | 0.026202665 | 1.0 | 0.694449936 | 0 |
| hsa-miR-206_st | 0.09481611 | 1.1 | 0.753900184 | 0 |
| hsa-miR-208a_st | 0.0523792 | 1.0 | 0.798140056 | 0 |
| hsa-miR-208b_st | 0.166898047 | 1.1 | 0.983201308 | 0 |
| hsa-miR-18b_st | 0.435054312 | 1.4 | 0.776510071 | 0 |
| hsa-miR-20a-star_st | 0.733406108 | 1.7 | 0.081693791 | 0 |
| hsa-miR-3181_st | −0.644449355 | 1.6 | 0.000745468 | 1 |
| hsa-miR-615-5p_st | −0.488241983 | 1.4 | 0.06986354 | 0 |
| hsa-miR-4259_st | −0.658221246 | 1.6 | 1.59656E−05 | 1 |
| hsa-miR-210_st | −0.616340673 | 1.5 | 3.77475E−05 | 1 |
| hsa-miR-211_st | −0.438423188 | 1.4 | 0.013250982 | 1 |
| hsa-miR-887_st | −0.500359564 | 1.4 | 0.008360023 | 1 |
| hsa-miR-2113_st | −0.048787149 | 1.0 | 0.704414214 | 0 |
| hsa-miR-2114_st | −0.24566565 | 1.2 | 0.123991393 | 0 |
| hsa-miR-943_st | −0.535162407 | 1.4 | 0.009522058 | 1 |
| hsa-miR-2115_st | 0.218427945 | 1.2 | 0.234226568 | 0 |
| hsa-miR-2115-star_st | −0.339479529 | 1.3 | 0.368650796 | 0 |
| hsa-miR-183-star_st | −0.327941515 | 1.3 | 0.01985218 | 1 |
| hsa-miR-2116-star_st | 0.050287017 | 1.0 | 0.704218708 | 0 |
| hsa-miR-920_st | −0.558662809 | 1.5 | 0.007714921 | 1 |
| hsa-miR-212_st | 0.340166135 | 1.3 | 0.003912012 | 1 |
| hsa-miR-214_st | −0.170059616 | 1.1 | 0.719896882 | 0 |
| hsa-miR-1825_st | −0.225066876 | 1.2 | 0.135432485 | 0 |
| hsa-miR-215_st | 0.470488353 | 1.4 | 0.442331812 | 0 |
| hsa-miR-216a_st | −0.060844105 | 1.0 | 0.575162441 | 0 |
| hsa-miR-195-star_st | −0.023958939 | 1.0 | 0.90489808 | 0 |
| hsa-miR-217_st | 0.17185971 | 1.1 | 0.176433368 | 0 |
| hsa-miR-23a-star_st | −0.428590827 | 1.3 | 0.031783886 | 1 |
| hsa-miR-25-star_st | −0.551507645 | 1.5 | 0.005143138 | 1 |
| hsa-miR-218-2-star_st | −0.175244052 | 1.1 | 0.271102398 | 0 |
| hsa-miR-1273c_st | −0.40064583 | 1.3 | 0.106881156 | 0 |
| hsa-miR-219-2-3p_st | −0.401983392 | 1.3 | 0.051868979 | 0 |
| hsa-miR-637_st | −0.653117025 | 1.6 | 0.00044811 | 1 |
| hsa-miR-21-star_st | 0.601654482 | 1.5 | 0.009817713 | 1 |
| hsa-miR-22_st | 0.796029353 | 1.7 | 0.014627165 | 1 |
| hsa-miR-220a_st | 0.24996554 | 1.2 | 0.096866346 | 0 |
| hsa-miR-220b_st | 0.250309064 | 1.2 | 0.123739258 | 0 |
| hsa-miR-219-1-3p_st | −0.281923306 | 1.2 | 0.024503465 | 1 |
| hsa-miR-4316_st | −0.480876791 | 1.4 | 0.001533793 | 1 |
| hsa-miR-877_st | −0.617502542 | 1.5 | 6.54248E−05 | 1 |
| hsa-miR-636_st | −0.49113505 | 1.4 | 0.006070819 | 1 |
| hsa-miR-3177_st | −0.657881548 | 1.6 | 0.000298485 | 1 |
| hsa-miR-1249_st | −0.046298628 | 1.0 | 0.848075712 | 0 |
| hsa-miR-223-star_st | 0.401799043 | 1.3 | 0.351859418 | 0 |
| hsa-miR-1224-5p_st | −0.6165197 | 1.5 | 6.71237E−06 | 1 |
| hsa-miR-564_st | −0.565311252 | 1.5 | 0.001267526 | 1 |
| hsa-miR-2276_st | −0.648220232 | 1.6 | 0.000721201 | 1 |
| hsa-miR-2277_st | −0.352678406 | 1.3 | 0.048673567 | 1 |
| hsa-miR-2278_st | −0.261848413 | 1.2 | 0.162059884 | 0 |
| hsa-miR-22-star_st | 1.062323014 | 2.1 | 0.013932188 | 1 |
| hsa-miR-2355_st | −0.093261401 | 1.1 | 0.290771967 | 0 |
| hsa-miR-769-3p_st | −0.489694573 | 1.4 | 0.037094124 | 1 |
| hsa-miR-1913_st | −0.362021722 | 1.3 | 0.084799625 | 0 |
| hsa-miR-1237_st | −0.275433027 | 1.2 | 0.171918458 | 0 |
| hsa-miR-1275_st | −0.467154658 | 1.4 | 0.007809435 | 1 |
| hsa-miR-30a_st | 0.994482288 | 2.0 | 0.00064785 | 1 |
| hsa-miR-20b-star_st | −0.270574253 | 1.2 | 0.01738856 | 1 |
| hsa-miR-24-2-star_st | 1.091725483 | 2.1 | 8.70722E−05 | 1 |
| hsa-miR-3173_st | −0.55033887 | 1.5 | 0.007085336 | 1 |
| hsa-miR-3148_st | −0.242944072 | 1.2 | 0.148633312 | 0 |
| hsa-miR-4271_st | −0.637545881 | 1.6 | 0.000425937 | 1 |
| hsa-miR-659_st | −0.492990393 | 1.4 | 0.00751997 | 1 |
| hsa-miR-26a-2-star_st | 0.145485123 | 1.1 | 0.900453716 | 0 |
| hsa-miR-1228_st | −0.472767718 | 1.4 | 0.012009445 | 1 |
| hsa-miR-26b-star_st | 0.868149649 | 1.8 | 0.003099131 | 1 |
| hsa-miR-3174_st | −0.160780973 | 1.1 | 0.55094482 | 0 |
| hsa-miR-27a-star_st | 0.108915254 | 1.1 | 0.650286341 | 0 |
| hsa-miR-1285_st | −0.359126421 | 1.3 | 0.0367001 | 1 |
| hsa-miR-483-5p_st | −0.448338126 | 1.4 | 0.026117133 | 1 |
| hsa-miR-1306_st | −0.364416168 | 1.3 | 0.003497236 | 1 |
| hsa-miR-431_st | 0.330128391 | 1.3 | 0.344039948 | 0 |
| hsa-miR-765_st | −0.417507121 | 1.3 | 0.024742559 | 1 |
| hsa-miR-2909_st | 0.204046548 | 1.2 | 0.213913136 | 0 |
| hsa-miR-296-3p_st | −0.529698688 | 1.4 | 0.00081683 | 1 |
| hsa-miR-296-5p_st | 0.205448097 | 1.2 | 0.435700668 | 0 |
| hsa-miR-297_st | −0.23324311 | 1.2 | 0.177544416 | 0 |
| hsa-miR-323b-5p_st | −0.158119612 | 1.1 | 0.78811908 | 0 |
| hsa-miR-299-3p_st | −0.205631118 | 1.2 | 0.503522303 | 0 |
| hsa-miR-299-5p_st | 0.092756749 | 1.1 | 0.971021124 | 0 |
| hsa-miR-487a_st | 0.362171988 | 1.3 | 0.046640739 | 1 |
| hsa-miR-29a-star_st | 0.038098103 | 1.0 | 0.846465338 | 0 |
| hsa-miR-625_st | 0.143664769 | 1.1 | 0.553203594 | 0 |
| hsa-miR-611_st | −0.347599942 | 1.3 | 0.139156229 | 0 |
| hsa-miR-3186-3p_st | 0.018134799 | 1.0 | 0.636818552 | 0 |
| hsa-miR-1294_st | −0.241617182 | 1.2 | 0.027633053 | 1 |
| hsa-miR-3147_st | −0.616379964 | 1.5 | 0.000114432 | 1 |
| hsa-miR-4285_st | −0.6688107 | 1.6 | 0.003880846 | 1 |
| hsa-miR-125a-3p_st | −0.338305912 | 1.3 | 0.025111823 | 1 |
| hsa-miR-301b_st | 0.157525213 | 1.1 | 0.960904014 | 0 |
| hsa-miR-302a_st | 0.044285746 | 1.0 | 0.989740982 | 0 |
| hsa-miR-518e-star_st | −0.314112434 | 1.2 | 0.235754188 | 0 |
| hsa-miR-302b_st | 0.04206039 | 1.0 | 0.863508675 | 0 |
| hsa-miR-302b-star_st | −0.193906932 | 1.1 | 0.14514732 | 0 |
| hsa-miR-302c_st | −0.020124281 | 1.0 | 0.65422486 | 0 |
| hsa-miR-302c-star_st | −0.564911045 | 1.5 | 0.03475367 | 1 |
| hsa-miR-302d_st | −0.038896466 | 1.0 | 0.39452106 | 0 |
| hsa-miR-302d-star_st | −0.27293483 | 1.2 | 0.533517783 | 0 |
| hsa-miR-302e_st | 0.137372123 | 1.1 | 0.646575145 | 0 |
| hsa-miR-302f_st | 0.174225731 | 1.1 | 0.322120676 | 0 |
| hsa-miR-3065-3p_st | 0.077698858 | 1.1 | 0.824427886 | 0 |
| hsa-miR-3130-5p_st | −0.301395577 | 1.2 | 0.08069423 | 0 |
| hsa-miR-3074_st | −0.139090182 | 1.1 | 0.353262151 | 0 |
| hsa-miR-1226-star_st | −0.554051488 | 1.5 | 0.000251982 | 1 |
| hsa-miR-3180-5p_st | −0.361808103 | 1.3 | 0.053512943 | 0 |
| hsa-miR-1229_st | 0.36309187 | 1.3 | 0.004726823 | 1 |
| hsa-miR-1272_st | −0.198223501 | 1.1 | 0.108708241 | 0 |
| hsa-miR-30c_st | 0.697390115 | 1.6 | 0.016837899 | 1 |
| hsa-miR-675_st | −0.498084265 | 1.4 | 0.005285191 | 1 |
| hsa-miR-30c-2-star_st | −0.094702821 | 1.1 | 0.828476533 | 0 |
| hsa-miR-1827_st | 0.226665792 | 1.2 | 0.701433956 | 0 |
| hsa-miR-30d-star_st | 0.295247972 | 1.2 | 0.093008682 | 0 |
| hsa-miR-30e_st | 1.030476483 | 2.0 | 0.005609173 | 1 |
| hsa-miR-1915-star_st | −0.166151902 | 1.1 | 0.498904668 | 0 |
| hsa-miR-1322_st | −0.143160741 | 1.1 | 0.315001603 | 0 |
| hsa-miR-3115_st | 0.162121437 | 1.1 | 0.363144244 | 0 |
| hsa-miR-3116_st | −0.334755156 | 1.3 | 0.283622071 | 0 |
| hsa-miR-3117_st | −0.116891649 | 1.1 | 0.458059946 | 0 |
| hsa-miR-3118_st | 0.139375712 | 1.1 | 0.665728467 | 0 |
| hsa-miR-519b-5p_st | −0.268159862 | 1.2 | 0.254280032 | 0 |
| hsa-miR-3120_st | −0.346659861 | 1.3 | 0.08683351 | 0 |
| hsa-miR-520d-3p_st | −0.20365475 | 1.2 | 0.229247768 | 0 |
| hsa-miR-326_st | 0.037758193 | 1.0 | 0.873102064 | 0 |
| hsa-miR-3123_st | 0.079620123 | 1.1 | 0.930829548 | 0 |
| hsa-miR-3124_st | −0.496047919 | 1.4 | 0.003106448 | 1 |
| hsa-miR-3125_st | −0.203379514 | 1.2 | 0.592674566 | 0 |
| hsa-miR-3126-3p_st | −0.273666886 | 1.2 | 0.06017569 | 0 |
| hsa-miR-1296_st | −0.132617848 | 1.1 | 0.827943007 | 0 |
| hsa-miR-3127_st | −0.593205109 | 1.5 | 0.010446767 | 1 |
| hsa-miR-3128_st | −0.057078556 | 1.0 | 0.372803813 | 0 |
| hsa-miR-3129_st | 0.073120444 | 1.1 | 0.826359967 | 0 |
| hsa-miR-3130-3p_st | −0.571898061 | 1.5 | 0.008660477 | 1 |
| hsa-miR-105-star_st | −0.169349295 | 1.1 | 0.105967677 | 0 |
| hsa-miR-3131_st | −0.481316892 | 1.4 | 0.008039702 | 1 |
| hsa-miR-4255_st | −0.175740033 | 1.1 | 0.051477643 | 0 |
| hsa-miR-3133_st | −0.123603968 | 1.1 | 0.449049667 | 0 |
| hsa-miR-3134_st | 0.019528486 | 1.0 | 0.786674222 | 0 |
| hsa-miR-3135_st | −0.42079135 | 1.3 | 0.023316049 | 1 |
| hsa-miR-3136_st | −0.357937798 | 1.3 | 0.010065854 | 1 |
| hsa-miR-19b-1-star_st | 0.244734498 | 1.2 | 0.345497789 | 0 |
| hsa-miR-1247_st | −0.521780044 | 1.4 | 0.027012999 | 1 |
| hsa-miR-3139_st | 0.05465175 | 1.0 | 0.949090377 | 0 |
| hsa-miR-596_st | −0.284551104 | 1.2 | 0.105768314 | 0 |
| hsa-miR-3141_st | −0.482399794 | 1.4 | 0.003310383 | 1 |
| hsa-miR-3142_st | 0.107763773 | 1.1 | 0.592268433 | 0 |
| hsa-miR-1302_st | −0.165059715 | 1.1 | 0.107099038 | 0 |
| hsa-miR-3144-3p_st | −0.044829172 | 1.0 | 0.502897584 | 0 |
| hsa-miR-3144-5p_st | −0.105193877 | 1.1 | 0.671849399 | 0 |
| hsa-miR-3145_st | −0.108921409 | 1.1 | 0.404567597 | 0 |
| hsa-miR-3146_st | −0.21066893 | 1.2 | 0.130181783 | 0 |
| hsa-miR-346_st | −0.301595597 | 1.2 | 0.134822409 | 0 |
| hsa-miR-4256_st | −0.284374753 | 1.2 | 0.004273465 | 1 |
| hsa-miR-3149_st | 0.087608242 | 1.1 | 0.812963268 | 0 |
| hsa-miR-3150_st | −0.353802881 | 1.3 | 0.12042593 | 0 |
| hsa-miR-3151_st | −0.555863627 | 1.5 | 0.003036503 | 1 |
| hsa-miR-1470_st | −0.218773405 | 1.2 | 0.166612242 | 0 |
| hsa-miR-3153_st | −0.456075181 | 1.4 | 0.073992293 | 0 |
| hsa-miR-514_st | 0.105466687 | 1.1 | 0.829731118 | 0 |
| hsa-miR-3175_st | −0.150334576 | 1.1 | 0.356003476 | 0 |
| hsa-miR-3156_st | −0.038231114 | 1.0 | 0.797486007 | 0 |
| hsa-miR-504_st | 0.058318416 | 1.0 | 0.52905772 | 0 |
| hsa-miR-3158_st | −0.125234901 | 1.1 | 0.238811109 | 0 |
| hsa-miR-3159_st | −0.229131179 | 1.2 | 0.124561401 | 0 |
| hsa-miR-3160_st | −0.139578186 | 1.1 | 0.377261574 | 0 |
| hsa-miR-3161_st | −0.191560704 | 1.1 | 0.708789425 | 0 |
| hsa-miR-3162_st | −0.513433751 | 1.4 | 0.000380219 | 1 |
| hsa-miR-1234_st | −0.257626876 | 1.2 | 0.122873601 | 0 |
| hsa-miR-3164_st | −0.667353407 | 1.6 | 0.003387283 | 1 |
| hsa-miR-516b-star_st | 0.23175777 | 1.2 | 0.089270532 | 0 |
| hsa-miR-3166_st | 0.136464153 | 1.1 | 0.221150448 | 0 |
| hsa-miR-4327_st | −0.646494192 | 1.6 | 6.73331E−05 | 1 |
| hsa-miR-3168_st | 0.155093847 | 1.1 | 0.470633398 | 0 |
| hsa-miR-3169_st | −0.19172817 | 1.1 | 0.030523306 | 1 |
| hsa-miR-3170_st | −0.32213791 | 1.3 | 0.149901365 | 0 |
| hsa-miR-3171_st | 0.044877251 | 1.0 | 0.770472338 | 0 |
| hsa-miR-3172_st | 0.316078382 | 1.2 | 0.042390621 | 1 |
| hsa-miR-374b-star_st | 0.324945805 | 1.3 | 0.106535636 | 0 |
| hsa-miR-34c-5p_st | 0.363927256 | 1.3 | 0.082737117 | 0 |
| hsa-miR-758_st | 0.033572974 | 1.0 | 0.770637393 | 0 |
| hsa-miR-3176_st | −0.089780423 | 1.1 | 0.783447529 | 0 |
| hsa-miR-606_st | 0.24109563 | 1.2 | 0.181705923 | 0 |
| hsa-miR-3178_st | −0.324916336 | 1.3 | 0.076234056 | 0 |
| hsa-miR-3179_st | −0.144469575 | 1.1 | 0.252248486 | 0 |
| hsa-miR-3180-3p_st | −0.39804147 | 1.3 | 0.022576113 | 1 |
| hsa-miR-597_st | 0.311808196 | 1.2 | 0.077619238 | 0 |
| hsa-miR-148b-star_st | 0.325395275 | 1.3 | 0.493046729 | 0 |
| hsa-miR-3182_st | 0.049297635 | 1.0 | 0.94256345 | 0 |
| hsa-miR-3183_st | −0.381333046 | 1.3 | 0.031748846 | 1 |
| hsa-miR-144-star_st | 0.245967497 | 1.2 | 0.817863664 | 0 |
| hsa-miR-3185_st | −0.42429146 | 1.3 | 0.006872335 | 1 |
| hsa-miR-4270_st | −0.395746034 | 1.3 | 0.002246656 | 1 |
| hsa-miR-3186-5p_st | −0.202034379 | 1.2 | 0.263945608 | 0 |
| hsa-miR-3187_st | −0.59552154 | 1.5 | 0.000752342 | 1 |
| hsa-miR-3188_st | −0.650448924 | 1.6 | 0.000148429 | 1 |
| hsa-miR-3189_st | −0.536216522 | 1.5 | 0.017541012 | 1 |
| hsa-miR-548b-3p_st | 0.165564469 | 1.1 | 0.357856676 | 0 |
| hsa-miR-99a-star_st | 0.378679583 | 1.3 | 0.029104819 | 1 |
| hsa-miR-376a-star_st | 0.143523874 | 1.1 | 0.754223064 | 0 |
| hsa-miR-216b_st | 0.33464651 | 1.3 | 0.079911971 | 0 |
| hsa-miR-3193_st | −0.117865737 | 1.1 | 0.339670933 | 0 |
| hsa-miR-548v_st | 0.181677341 | 1.1 | 0.383514414 | 0 |
| hsa-miR-26a-1-star_st | −0.113022689 | 1.1 | 0.142542744 | 0 |
| hsa-miR-1291_st | −0.188694808 | 1.1 | 0.558761121 | 0 |
| hsa-miR-3197_st | −0.416280922 | 1.3 | 0.031470741 | 1 |
| hsa-miR-629_st | 0.53059199 | 1.4 | 0.047136654 | 1 |
| hsa-miR-3199_st | −0.073753948 | 1.1 | 0.752895194 | 0 |
| hsa-miR-190b_st | 0.150853396 | 1.1 | 0.467457706 | 0 |
| hsa-miR-32_st | 0.379471614 | 1.3 | 0.324251922 | 0 |
| hsa-miR-3200_st | 0.39681502 | 1.3 | 0.594993034 | 0 |
| hsa-miR-3201_st | −0.197132532 | 1.1 | 0.141976926 | 0 |
| hsa-miR-3202_st | −0.165833823 | 1.1 | 0.75062254 | 0 |
| hsa-miR-320a_st | 0.031341695 | 1.0 | 0.739224092 | 0 |
| hsa-miR-4289_st | 0.305557709 | 1.2 | 0.476379713 | 0 |
| hsa-miR-655_st | 0.275794223 | 1.2 | 0.199105615 | 0 |
| hsa-miR-320b_st | 0.263822749 | 1.2 | 0.390287903 | 0 |
| hsa-miR-219-5p_st | 0.32310387 | 1.3 | 0.135969301 | 0 |
| hsa-miR-323-3p_st | 0.102714784 | 1.1 | 0.568869503 | 0 |
| hsa-miR-323-5p_st | −0.677397776 | 1.6 | 0.002057983 | 1 |
| hsa-miR-323b-3p_st | −0.063146339 | 1.0 | 0.332946797 | 0 |
| hsa-miR-103-as_st | 0.446114831 | 1.4 | 0.406509695 | 0 |
| hsa-miR-424-star_st | 0.409370915 | 1.3 | 0.01747411 | 1 |
| hsa-miR-33a-star_st | 0.702410697 | 1.6 | 0.003440344 | 1 |
| hsa-miR-325_st | −0.29161421 | 1.2 | 0.144937243 | 0 |
| hsa-miR-590-3p_st | 0.053234507 | 1.0 | 0.996316858 | 0 |
| hsa-miR-328_st | 0.484581153 | 1.4 | 0.04436787 | 1 |
| hsa-miR-329_st | 0.468923974 | 1.4 | 0.261414375 | 0 |
| hsa-miR-32-star_st | −0.002071102 | 1.0 | 0.837603528 | 0 |
| hsa-miR-330-3p_st | 0.108227539 | 1.1 | 0.495598243 | 0 |
| hsa-miR-330-5p_st | −0.248644944 | 1.2 | 0.132529571 | 0 |
| hsa-miR-146a-star_st | 0.425698974 | 1.3 | 0.007237824 | 1 |
| hsa-miR-331-5p_st | 0.304603187 | 1.2 | 0.198622198 | 0 |
| hsa-miR-3167_st | −0.15042957 | 1.1 | 0.132278021 | 0 |
| hsa-miR-335-star_st | 0.033221219 | 1.0 | 0.948001108 | 0 |
| hsa-miR-337-3p_st | −0.206645095 | 1.2 | 0.211291805 | 0 |
| hsa-miR-337-5p_st | 0.087872207 | 1.1 | 0.936031535 | 0 |
| hsa-miR-421_st | 0.756107095 | 1.7 | 0.005915337 | 1 |
| hsa-miR-338-5p_st | 0.385981843 | 1.3 | 0.019881365 | 1 |
| hsa-miR-339-3p_st | 0.242986305 | 1.2 | 0.364644233 | 0 |
| hsa-miR-548a-5p_st | 0.250044053 | 1.2 | 0.075005341 | 0 |
| hsa-miR-33a_st | 0.079184898 | 1.1 | 0.893015221 | 0 |
| hsa-miR-590-5p_st | 0.497732874 | 1.4 | 0.113867735 | 0 |
| hsa-miR-33b_st | 0.164476591 | 1.1 | 0.386739804 | 0 |
| hsa-miR-616-star_st | 0.360624151 | 1.3 | 0.188910655 | 0 |
| hsa-miR-569_st | 0.044737942 | 1.0 | 0.948954854 | 0 |
| hsa-miR-340-star_st | 0.23734343 | 1.2 | 0.822883424 | 0 |
| hsa-miR-342-3p_st | 0.916270247 | 1.9 | 0.007606699 | 1 |
| hsa-miR-135b-star_st | −0.240589471 | 1.2 | 0.494181719 | 0 |
| hsa-miR-345_st | 0.537798944 | 1.5 | 0.128822349 | 0 |
| hsa-miR-1288_st | −0.306269153 | 1.2 | 0.010579268 | 1 |
| hsa-miR-135a_st | 0.332346382 | 1.3 | 0.167518586 | 0 |
| hsa-miR-34a-star_st | 1.412262908 | 2.7 | 4.62432E−06 | 1 |
| hsa-miR-34b_st | 0.075498263 | 1.1 | 0.771732268 | 0 |
| hsa-miR-4288_st | 0.541843179 | 1.5 | 0.000322195 | 1 |
| hsa-miR-222-star_st | 1.619255569 | 3.1 | 6.5231E−08 | 1 |
| hsa-miR-933_st | 0.517830677 | 1.4 | 0.000488625 | 1 |
| hsa-miR-361-3p_st | 0.893085031 | 1.9 | 0.002838072 | 1 |
| hsa-miR-224-star_st | 0.650757144 | 1.6 | 7.18153E−05 | 1 |
| hsa-miR-362-3p_st | 0.611487126 | 1.5 | 0.113774191 | 0 |
| hsa-miR-362-5p_st | 0.812726568 | 1.8 | 0.035038911 | 1 |
| hsa-miR-363_st | 0.300075295 | 1.2 | 0.899375362 | 0 |
| hsa-miR-363-star_st | −0.411600303 | 1.3 | 0.096372894 | 0 |
| hsa-miR-1979_st | 0.234834332 | 1.2 | 0.15559618 | 0 |
| hsa-miR-365-star_st | −0.65784463 | 1.6 | 2.95972E−05 | 1 |
| hsa-miR-548x_st | 0.191663608 | 1.1 | 0.473809154 | 0 |
| hsa-miR-367-star_st | 0.228618965 | 1.2 | 0.444389 | 0 |
| hsa-miR-369-3p_st | 0.079192264 | 1.1 | 0.938584045 | 0 |
| hsa-miR-369-5p_st | 0.38370611 | 1.3 | 0.284930739 | 0 |
| hsa-miR-548l_st | −0.357788449 | 1.3 | 0.003068723 | 1 |
| hsa-miR-371-3p_st | 0.028118225 | 1.0 | 0.896624958 | 0 |
| hsa-miR-371-5p_st | −0.675633424 | 1.6 | 0.000284331 | 1 |
| hsa-miR-372_st | −0.209821004 | 1.2 | 0.100365975 | 0 |
| hsa-miR-373_st | −0.200926298 | 1.1 | 0.150770571 | 0 |
| hsa-miR-373-star_st | −0.703405456 | 1.6 | 0.000549114 | 1 |
| hsa-miR-374a_st | 0.926305331 | 1.9 | 0.03393499 | 1 |
| hsa-miR-374a-star_st | 0.305877302 | 1.2 | 0.180851549 | 0 |
| hsa-miR-500_st | 0.544053308 | 1.5 | 0.123353807 | 0 |
| hsa-miR-320c_st | 0.384686554 | 1.3 | 0.23156477 | 0 |
| hsa-miR-875-3p_st | −0.100148108 | 1.1 | 0.548997441 | 0 |
| hsa-miR-376a_st | 0.243165228 | 1.2 | 0.446619806 | 0 |
| hsa-miR-29b-1-star_st | 1.381274558 | 2.6 | 1.06486E−08 | 1 |
| hsa-miR-376b_st | 0.196773499 | 1.1 | 0.482988609 | 0 |
| hsa-miR-376c_st | 0.476501212 | 1.4 | 0.349211393 | 0 |
| hsa-miR-377_st | 0.04502713 | 1.0 | 0.817004286 | 0 |
| hsa-miR-377-star_st | 0.10112772 | 1.1 | 0.572753614 | 0 |
| hsa-miR-378_st | 0.196431612 | 1.1 | 0.614381174 | 0 |
| hsa-miR-378b_st | −0.359615329 | 1.3 | 0.001544915 | 1 |
| hsa-miR-378c_st | 0.003465018 | 1.0 | 0.774234835 | 0 |
| hsa-miR-378-star_st | 0.213515147 | 1.2 | 0.680533548 | 0 |
| hsa-miR-662_st | 0.763588292 | 1.7 | 1.07501E−05 | 1 |
| hsa-miR-379-star_st | 0.227503468 | 1.2 | 0.299168737 | 0 |
| hsa-miR-380_st | 0.097700397 | 1.1 | 0.616418601 | 0 |
| hsa-miR-380-star_st | −0.013558542 | 1.0 | 0.603823228 | 0 |
| hsa-miR-1274b_st | 0.845393159 | 1.8 | 8.83277E−06 | 1 |
| hsa-miR-382_st | −0.054872541 | 1.0 | 0.471142984 | 0 |
| hsa-miR-383_st | −0.241581552 | 1.2 | 0.230492847 | 0 |
| hsa-miR-384_st | 0.149079123 | 1.1 | 0.551389794 | 0 |
| hsa-miR-126-star_st | 0.089161991 | 1.1 | 0.813213299 | 0 |
| hsa-miR-409-5p_st | 0.057478949 | 1.0 | 0.726066758 | 0 |
| hsa-miR-410_st | 0.119761141 | 1.1 | 0.924713955 | 0 |
| hsa-miR-411_st | 0.035735668 | 1.0 | 0.947773657 | 0 |
| hsa-miR-411-star_st | 0.233518908 | 1.2 | 0.274393389 | 0 |
| hsa-miR-412_st | −0.230455194 | 1.2 | 0.094585458 | 0 |
| hsa-miR-3143_st | −0.049299534 | 1.0 | 0.505837404 | 0 |
| hsa-miR-422a_st | −0.002713527 | 1.0 | 0.663973065 | 0 |
| hsa-miR-628-5p_st | 0.439025551 | 1.4 | 0.151445525 | 0 |
| hsa-miR-423-5p_st | −0.404595069 | 1.3 | 4.1287E−05 | 1 |
| hsa-miR-27b-star_st | 1.345333447 | 2.5 | 6.12598E−11 | 1 |
| hsa-miR-499-5p_st | 0.592704906 | 1.5 | 0.001377201 | 1 |
| hsa-miR-425_st | 1.46433333 | 2.8 | 0.002796431 | 1 |
| hsa-miR-193b_st | 0.507490483 | 1.4 | 0.040473273 | 1 |
| hsa-miR-4252_st | −0.172298982 | 1.1 | 0.148194693 | 0 |
| hsa-miR-4253_st | −0.540168527 | 1.5 | 0.002206523 | 1 |
| hsa-miR-4254_st | 0.377577983 | 1.3 | 0.011427783 | 1 |
| hsa-miR-324-3p_st | 0.366781202 | 1.3 | 0.274311175 | 0 |
| hsa-miR-4329_st | 0.584404594 | 1.5 | 2.34854E−05 | 1 |
| hsa-miR-1251_st | 0.712759923 | 1.6 | 0.000898935 | 1 |
| hsa-miR-4258_st | −0.265408362 | 1.2 | 0.101479443 | 0 |
| hsa-miR-1274a_st | 1.396590701 | 2.6 | 9.66639E−06 | 1 |
| hsa-miR-425-star_st | −0.035561492 | 1.0 | 0.368955534 | 0 |
| hsa-miR-4260_st | −0.549233748 | 1.5 | 0.003101776 | 1 |
| hsa-miR-4261_st | −0.131817236 | 1.1 | 0.947174184 | 0 |
| hsa-miR-4262_st | −0.100446121 | 1.1 | 0.190462159 | 0 |
| hsa-miR-4263_st | 0.022331211 | 1.0 | 0.706219063 | 0 |
| hsa-miR-4264_st | 0.046291164 | 1.0 | 0.992089982 | 0 |
| hsa-miR-1280_st | 0.531015773 | 1.4 | 0.002892407 | 1 |
| hsa-miR-132-star_st | 1.193843307 | 2.3 | 5.16813E−05 | 1 |
| hsa-miR-4267_st | −0.31842273 | 1.2 | 0.086827512 | 0 |
| hsa-miR-4268_st | −0.022608683 | 1.0 | 0.638667513 | 0 |
| hsa-miR-133a_st | 0.26043092 | 1.2 | 0.488072584 | 0 |
| hsa-miR-888-star_st | 0.069768899 | 1.0 | 0.878560963 | 0 |
| hsa-miR-1260b_st | 0.56665893 | 1.5 | 0.001477379 | 1 |
| hsa-miR-4272_st | 0.047875654 | 1.0 | 0.922564818 | 0 |
| hsa-miR-4273_st | 0.008323529 | 1.0 | 0.949193841 | 0 |
| hsa-miR-4274_st | 0.031306372 | 1.0 | 0.654021907 | 0 |
| hsa-miR-4275_st | 0.274356152 | 1.2 | 0.193038743 | 0 |
| hsa-miR-4276_st | 0.149602497 | 1.1 | 0.792683539 | 0 |
| hsa-miR-4277_st | −0.028296434 | 1.0 | 0.468126529 | 0 |
| hsa-miR-4278_st | −0.33927234 | 1.3 | 0.157634019 | 0 |
| hsa-miR-4279_st | −0.051171417 | 1.0 | 0.775988362 | 0 |
| hsa-miR-4280_st | −0.144129929 | 1.1 | 0.426319175 | 0 |
| hsa-miR-4281_st | −0.245192821 | 1.2 | 0.10169252 | 0 |
| hsa-miR-4282_st | 0.0883115 | 1.1 | 0.714236975 | 0 |
| hsa-miR-4283_st | −0.523762966 | 1.4 | 0.008304566 | 1 |
| hsa-miR-4284_st | 0.289625456 | 1.2 | 0.067562954 | 0 |
| hsa-miR-23b-star_st | 0.674734444 | 1.6 | 0.00016754 | 1 |
| hsa-miR-574-3p_st | 0.493680235 | 1.4 | 0.016968497 | 1 |
| hsa-miR-4287_st | −0.330389027 | 1.3 | 0.05809358 | 0 |
| hsa-miR-320e_st | 0.658151384 | 1.6 | 0.037311305 | 1 |
| hsa-miR-339-5p_st | 1.068143508 | 2.1 | 0.003493607 | 1 |
| hsa-miR-505_st | 0.816928161 | 1.8 | 0.013571202 | 1 |
| hsa-miR-4290_st | −0.074745651 | 1.1 | 0.917624577 | 0 |
| hsa-miR-200b-star_st | 0.472418518 | 1.4 | 0.010314667 | 1 |
| hsa-miR-4292_st | −0.561491115 | 1.5 | 0.002877083 | 1 |
| hsa-miR-4293_st | −0.195543258 | 1.1 | 0.129949588 | 0 |
| hsa-miR-1246_st | 0.448320609 | 1.4 | 0.002204115 | 1 |
| hsa-miR-4295_st | 0.087703996 | 1.1 | 0.769149115 | 0 |
| hsa-miR-4296_st | −0.42212698 | 1.3 | 0.041649355 | 1 |
| hsa-miR-4297_st | −0.222550583 | 1.2 | 0.364173748 | 0 |
| hsa-miR-4298_st | −0.509384082 | 1.4 | 0.000238858 | 1 |
| hsa-miR-218_st | 1.531746761 | 2.9 | 2.68386E−06 | 1 |
| hsa-miR-4286_st | 0.986581034 | 2.0 | 7.793E−05 | 1 |
| hsa-miR-4301_st | 0.246302895 | 1.2 | 0.209320166 | 0 |
| hsa-miR-4302_st | −0.39810134 | 1.3 | 0.029726087 | 1 |
| hsa-miR-365_st | 0.322524305 | 1.3 | 0.155233838 | 0 |
| hsa-miR-4304_st | −0.325010347 | 1.3 | 0.131393604 | 0 |
| hsa-miR-4305_st | 0.131987059 | 1.1 | 0.629483336 | 0 |
| hsa-miR-4306_st | 0.18368738 | 1.1 | 0.906703202 | 0 |
| hsa-miR-26a_st | 0.731517215 | 1.7 | 0.000973168 | 1 |
| hsa-miR-4308_st | 0.126508126 | 1.1 | 0.726031428 | 0 |
| hsa-miR-4309_st | 0.11828394 | 1.1 | 0.301547703 | 0 |
| hsa-miR-186_st | 0.981928548 | 2.0 | 0.017912001 | 1 |
| hsa-miR-4310_st | −0.318253176 | 1.2 | 0.185655282 | 0 |
| hsa-miR-4311_st | −0.099894225 | 1.1 | 0.508741816 | 0 |
| hsa-miR-4312_st | −0.319187789 | 1.2 | 0.059733035 | 0 |
| hsa-miR-4313_st | 0.057807986 | 1.0 | 0.533807726 | 0 |
| hsa-miR-4314_st | −0.573075857 | 1.5 | 0.074819139 | 0 |
| hsa-miR-4315_st | −0.045208689 | 1.0 | 0.778907653 | 0 |
| hsa-miR-149_st | 0.991769572 | 2.0 | 2.07887E−05 | 1 |
| hsa-miR-106b-star_st | 0.756887645 | 1.7 | 0.081496092 | 0 |
| hsa-miR-4318_st | −0.125790696 | 1.1 | 0.114713029 | 0 |
| hsa-miR-4319_st | 3.3607E−05 | 1.0 | 0.721561227 | 0 |
| hsa-miR-29b-2-star_st | 0.970878605 | 2.0 | 0.000691297 | 1 |
| hsa-miR-432_st | −0.424110692 | 1.3 | 0.050867914 | 0 |
| hsa-miR-4320_st | 0.141840951 | 1.1 | 0.305275801 | 0 |
| hsa-miR-522_st | 0.038498664 | 1.0 | 0.94513551 | 0 |
| hsa-miR-4322_st | −0.623367889 | 1.5 | 0.000138789 | 1 |
| hsa-miR-4323_st | −0.164242896 | 1.1 | 0.102224887 | 0 |
| hsa-miR-93-star_st | 0.7694271 | 1.7 | 0.094194312 | 0 |
| hsa-miR-4325_st | 0.227381131 | 1.2 | 0.216657261 | 0 |
| hsa-miR-4326_st | 0.008303948 | 1.0 | 0.423165271 | 0 |
| hsa-miR-874_st | −0.503966615 | 1.4 | 0.000346609 | 1 |
| hsa-miR-4328_st | 0.122228922 | 1.1 | 0.641831652 | 0 |
| hsa-let-7e-star_st | 0.838867983 | 1.8 | 9.47634E−06 | 1 |
| hsa-miR-720_st | 0.159440883 | 1.1 | 0.231843963 | 0 |
| hsa-miR-433_st | −0.297351442 | 1.2 | 0.10290867 | 0 |
| hsa-miR-4330_st | −0.098292553 | 1.1 | 0.728722347 | 0 |
| hsa-miR-448_st | −0.085705823 | 1.1 | 0.426042536 | 0 |
| hsa-miR-449a_st | 0.366580686 | 1.3 | 0.00793599 | 1 |
| hsa-miR-449b_st | 0.023835538 | 1.0 | 0.984070323 | 0 |
| hsa-miR-1975_st | 0.065733098 | 1.0 | 0.64181964 | 0 |
| hsa-miR-449c_st | −0.614519733 | 1.5 | 0.023372257 | 1 |
| hsa-miR-449c-star_st | −0.121362017 | 1.1 | 0.35285535 | 0 |
| hsa-miR-1_st | 0.576677084 | 1.5 | 0.175040016 | 0 |
| hsa-miR-450b-3p_st | 0.062981579 | 1.0 | 0.940591891 | 0 |
| hsa-miR-450b-5p_st | 0.131221455 | 1.1 | 0.738380499 | 0 |
| hsa-miR-451_st | 0.087909523 | 1.1 | 0.902828672 | 0 |
| hsa-miR-452_st | 0.14628175 | 1.1 | 0.276965995 | 0 |
| hsa-miR-452-star_st | −0.013845816 | 1.0 | 0.524568582 | 0 |
| hsa-miR-548b-5p_st | −0.223673286 | 1.2 | 0.02301896 | 1 |
| hsa-miR-454-star_st | 0.153592273 | 1.1 | 0.709524442 | 0 |
| hsa-miR-455-3p_st | 0.429029588 | 1.3 | 0.03265621 | 1 |
| hsa-miR-455-5p_st | 0.334081936 | 1.3 | 0.114091363 | 0 |
| hsa-miR-466_st | 0.237582262 | 1.2 | 0.274043516 | 0 |
| hsa-miR-483-3p_st | −0.003579204 | 1.0 | 0.673764763 | 0 |
| hsa-miR-17_st | 1.280302358 | 2.4 | 0.002663743 | 1 |
| hsa-miR-320d_st | 0.609805751 | 1.5 | 0.081568607 | 0 |
| hsa-miR-485-3p_st | 0.051314079 | 1.0 | 0.714077809 | 0 |
| hsa-miR-485-5p_st | −0.374240628 | 1.3 | 0.075836232 | 0 |
| hsa-miR-486-3p_st | −0.479726394 | 1.4 | 0.009009035 | 1 |
| hsa-miR-486-5p_st | −0.516559938 | 1.4 | 0.073247071 | 0 |
| hsa-miR-644_st | −0.021364394 | 1.0 | 0.607886124 | 0 |
| hsa-miR-3152_st | −0.645703485 | 1.6 | 0.013781726 | 1 |
| hsa-miR-488_st | 0.17051326 | 1.1 | 0.309623859 | 0 |
| hsa-miR-488-star_st | 0.020035891 | 1.0 | 0.854491308 | 0 |
| hsa-miR-450a_st | 1.177111428 | 2.3 | 1.62434E−05 | 1 |
| hsa-miR-20a_st | 1.52088829 | 2.9 | 0.00108399 | 1 |
| hsa-miR-484_st | 0.779514219 | 1.7 | 0.096639651 | 0 |
| hsa-miR-491-3p_st | 0.207398076 | 1.2 | 0.368163528 | 0 |
| hsa-miR-491-5p_st | −0.038602201 | 1.0 | 0.967909058 | 0 |
| hsa-miR-197_st | 0.66899893 | 1.6 | 0.041442563 | 1 |
| hsa-miR-493_st | 0.060363307 | 1.0 | 0.517703068 | 0 |
| hsa-miR-493-star_st | 0.393211268 | 1.3 | 0.309068907 | 0 |
| hsa-miR-494_st | −0.517544894 | 1.4 | 0.009758189 | 1 |
| hsa-miR-495_st | 0.589319919 | 1.5 | 0.25588984 | 0 |
| hsa-miR-496_st | 0.145656451 | 1.1 | 0.79618088 | 0 |
| hsa-miR-497_st | 0.251078501 | 1.2 | 0.236697173 | 0 |
| hsa-miR-497-star_st | 0.098309609 | 1.1 | 0.769168398 | 0 |
| hsa-miR-498_st | −0.632854392 | 1.6 | 0.000321265 | 1 |
| hsa-miR-499-3p_st | 0.187477685 | 1.1 | 0.328365055 | 0 |
| hsa-miR-24-1-star_st | 1.073684055 | 2.1 | 0.000145774 | 1 |
| hsa-miR-340_st | 1.277302225 | 2.4 | 0.000254287 | 1 |
| hsa-miR-500b_st | 0.294745175 | 1.2 | 0.606052116 | 0 |
| hsa-miR-500-star_st | −0.176098719 | 1.1 | 0.15677611 | 0 |
| hsa-miR-501-3p_st | −0.295874074 | 1.2 | 0.011517777 | 1 |
| hsa-miR-501-5p_st | 0.028766386 | 1.0 | 0.564678845 | 0 |
| hsa-miR-502-3p_st | −0.181888519 | 1.1 | 0.245192366 | 0 |
| hsa-miR-502-5p_st | 0.018519743 | 1.0 | 0.526147604 | 0 |
| hsa-miR-503_st | −0.395058426 | 1.3 | 0.012938435 | 1 |
| hsa-miR-192_st | 1.408219456 | 2.7 | 0.001683851 | 1 |
| hsa-let-7g_st | 0.951098893 | 1.9 | 0.000556005 | 1 |
| hsa-miR-505-star_st | −0.117754301 | 1.1 | 0.412878727 | 0 |
| hsa-miR-506_st | 0.08018806 | 1.1 | 0.650324727 | 0 |
| hsa-miR-507_st | 0.107076115 | 1.1 | 0.784908678 | 0 |
| hsa-miR-508-3p_st | 0.171625966 | 1.1 | 0.510074082 | 0 |
| hsa-miR-508-5p_st | −0.409707488 | 1.3 | 0.08539915 | 0 |
| hsa-miR-509-3-5p_st | −0.224948007 | 1.2 | 0.435423506 | 0 |
| hsa-miR-509-3p_st | −0.198270923 | 1.1 | 0.237548418 | 0 |
| hsa-miR-106a_st | 1.387442345 | 2.6 | 0.001376282 | 1 |
| hsa-miR-106b_st | 1.503196879 | 2.8 | 0.000594296 | 1 |
| hsa-miR-511_st | −0.05786449 | 1.0 | 0.470208528 | 0 |
| hsa-miR-512-3p_st | −0.300259224 | 1.2 | 0.025341252 | 1 |
| hsa-miR-512-5p_st | −0.189876727 | 1.1 | 0.327500962 | 0 |
| hsa-miR-513a-3p_st | 0.147832753 | 1.1 | 0.90217688 | 0 |
| hsa-miR-23a_st | 0.884694686 | 1.8 | 0.000352968 | 1 |
| hsa-miR-25_st | 1.185057753 | 2.3 | 0.002512048 | 1 |
| hsa-miR-598_st | 1.226426823 | 2.3 | 0.000111603 | 1 |
| hsa-miR-1260_st | 1.048914152 | 2.1 | 1.05371E−05 | 1 |
| hsa-miR-514b-3p_st | 0.031479132 | 1.0 | 0.807900103 | 0 |
| hsa-miR-514b-5p_st | −0.433113987 | 1.4 | 0.084324372 | 0 |
| hsa-miR-515-3p_st | −0.124604022 | 1.1 | 0.250116715 | 0 |
| hsa-miR-515-5p_st | −0.064987586 | 1.0 | 0.448814814 | 0 |
| hsa-miR-516a-3p_st | 0.067879583 | 1.0 | 0.543462019 | 0 |
| hsa-miR-27a_st | 1.343744849 | 2.5 | 1.84384E−05 | 1 |
| hsa-miR-100_st | 0.702747422 | 1.6 | 0.003396779 | 1 |
| hsa-miR-101_st | 1.471342647 | 2.8 | 0.002391542 | 1 |
| hsa-miR-517a_st | 0.194954754 | 1.1 | 0.425874486 | 0 |
| hsa-miR-517b_st | 0.156034295 | 1.1 | 0.372704275 | 0 |
| hsa-miR-517c_st | 0.047605038 | 1.0 | 0.896054297 | 0 |
| hsa-miR-517-star_st | −0.107791491 | 1.1 | 0.554795002 | 0 |
| hsa-miR-518a-3p_st | −0.094504876 | 1.1 | 0.256203316 | 0 |
| hsa-miR-518a-5p_st | −0.413729016 | 1.3 | 0.187456262 | 0 |
| hsa-miR-518b_st | 0.088807039 | 1.1 | 0.528113313 | 0 |
| hsa-miR-518c_st | 0.099106728 | 1.1 | 0.656628824 | 0 |
| hsa-miR-301a_st | 1.229986909 | 2.3 | 0.007070412 | 1 |
| hsa-miR-518d-3p_st | −0.070042193 | 1.0 | 0.448313542 | 0 |
| hsa-miR-518d-5p_st | −0.112868354 | 1.1 | 0.953533136 | 0 |
| hsa-miR-518e_st | 0.292065323 | 1.2 | 0.082091477 | 0 |
| hsa-let-7f_st | 0.782597495 | 1.7 | 0.000877066 | 1 |
| hsa-miR-518f_st | 0.10510646 | 1.1 | 0.815781034 | 0 |
| hsa-miR-1826_st | −0.039286991 | 1.0 | 0.726221744 | 0 |
| hsa-miR-519a_st | −0.045402557 | 1.0 | 0.461383253 | 0 |
| hsa-miR-519a-star_st | −0.382392357 | 1.3 | 0.099584817 | 0 |
| hsa-miR-519b-3p_st | 0.092177111 | 1.1 | 0.798480341 | 0 |
| hsa-miR-1283_st | −0.152770096 | 1.1 | 0.101080096 | 0 |
| hsa-miR-519c-3p_st | 0.012292479 | 1.0 | 0.655642874 | 0 |
| hsa-miR-519c-5p_st | −0.289621284 | 1.2 | 0.325886895 | 0 |
| hsa-miR-519d_st | 0.127516999 | 1.1 | 0.48000076 | 0 |
| hsa-miR-519e_st | 0.427023433 | 1.3 | 0.002801606 | 1 |
| hsa-miR-519e-star_st | −0.392432506 | 1.3 | 0.197229694 | 0 |
| hsa-miR-520a-3p_st | 0.073997868 | 1.1 | 0.760236215 | 0 |
| hsa-miR-1908_st | −0.048787304 | 1.0 | 0.71747425 | 0 |
| hsa-miR-520b_st | 0.096423039 | 1.1 | 0.977921461 | 0 |
| hsa-miR-520c-3p_st | 0.104001753 | 1.1 | 0.92273025 | 0 |
| hsa-miR-520c-5p_st | −0.04378828 | 1.0 | 0.741518073 | 0 |
| hsa-miR-181a-star_st | 1.231483023 | 2.3 | 2.86829E−05 | 1 |
| hsa-miR-21_st | 2.403298997 | 5.3 | 2.52274E−07 | 1 |
| hsa-miR-520e_st | −0.020382845 | 1.0 | 0.742603812 | 0 |
| hsa-miR-520f_st | 0.271286942 | 1.2 | 0.103461226 | 0 |
| hsa-miR-520g_st | 0.069632427 | 1.0 | 0.962905246 | 0 |
| hsa-miR-520h_st | 0.068680109 | 1.0 | 0.636074311 | 0 |
| hsa-miR-521_st | 0.330682833 | 1.3 | 0.035773465 | 1 |
| hsa-miR-302a-star_st | −0.136094511 | 1.1 | 0.104813388 | 0 |
| hsa-miR-886-3p_st | 0.905098806 | 1.9 | 0.002050876 | 1 |
| hsa-miR-523_st | 0.027147295 | 1.0 | 0.844981122 | 0 |
| hsa-miR-29a_st | 1.375508718 | 2.6 | 2.88816E−06 | 1 |
| hsa-miR-524-3p_st | −0.198145779 | 1.1 | 0.086716632 | 0 |
| hsa-miR-142-3p_st | −0.011017493 | 1.0 | 0.388997211 | 0 |
| hsa-miR-525-3p_st | −0.211180947 | 1.2 | 0.068652678 | 0 |
| hsa-miR-525-5p_st | −0.098964363 | 1.1 | 0.737175826 | 0 |
| hsa-miR-762_st | −0.081948997 | 1.1 | 0.556859762 | 0 |
| hsa-miR-423-3p_st | 0.970758984 | 2.0 | 0.00014707 | 1 |
| hsa-miR-449b-star_st | −0.34371066 | 1.3 | 0.173898667 | 0 |
| hsa-miR-527_st | −0.412065956 | 1.3 | 0.125421726 | 0 |
| hsa-miR-532-3p_st | 0.436379865 | 1.4 | 0.276500673 | 0 |
| hsa-miR-532-5p_st | 0.560594636 | 1.5 | 0.115791549 | 0 |
| hsa-miR-539_st | 0.192944212 | 1.1 | 0.393435942 | 0 |
| hsa-let-7a_st | 0.727952266 | 1.7 | 0.000940984 | 1 |
| hsa-miR-541-star_st | −0.228586147 | 1.2 | 0.143001924 | 0 |
| hsa-miR-542-3p_st | 0.272885547 | 1.2 | 0.317437786 | 0 |
| hsa-miR-542-5p_st | 0.687207164 | 1.6 | 9.68402E−06 | 1 |
| hsa-miR-543_st | 0.042876484 | 1.0 | 0.64976712 | 0 |
| hsa-miR-544_st | 0.041712614 | 1.0 | 0.86364095 | 0 |
| hsa-miR-544b_st | −0.138680853 | 1.1 | 0.28945059 | 0 |
| hsa-miR-545_st | 0.014502165 | 1.0 | 0.831334779 | 0 |
| hsa-miR-545-star_st | 0.272930768 | 1.2 | 0.254388135 | 0 |
| hsa-miR-548a-3p_st | 0.081691307 | 1.1 | 0.996125063 | 0 |
| hsa-miR-2861_st | −0.074130242 | 1.1 | 0.594353575 | 0 |
| hsa-miR-31_st | 2.756212833 | 6.8 | 1.62816E−05 | 1 |
| hsa-miR-367_st | −0.025354841 | 1.0 | 0.728359058 | 0 |
| hsa-miR-548c-3p_st | 0.304646502 | 1.2 | 0.240803422 | 0 |
| hsa-miR-548c-5p_st | 0.205390424 | 1.2 | 0.247886209 | 0 |
| hsa-miR-548d-3p_st | 0.114750589 | 1.1 | 0.745723133 | 0 |
| hsa-miR-548d-5p_st | 0.103099793 | 1.1 | 0.602511689 | 0 |
| hsa-miR-548e_st | 0.031213042 | 1.0 | 0.835974584 | 0 |
| hsa-miR-548f_st | 0.048760585 | 1.0 | 0.927328557 | 0 |
| hsa-miR-548g_st | 0.142662826 | 1.1 | 0.663781925 | 0 |
| hsa-miR-548h_st | 0.363589905 | 1.3 | 0.02502833 | 1 |
| hsa-miR-548i_st | 0.040552783 | 1.0 | 0.848917438 | 0 |
| hsa-miR-548j_st | −0.113264276 | 1.1 | 0.172409604 | 0 |
| hsa-miR-548k_st | 0.058910016 | 1.0 | 0.585610341 | 0 |
| hsa-miR-526b-star_st | −0.022783961 | 1.0 | 0.606339957 | 0 |
| hsa-miR-548m_st | −0.069590121 | 1.0 | 0.587856834 | 0 |
| hsa-miR-548n_st | 0.194176229 | 1.1 | 0.429688871 | 0 |
| hsa-miR-548o_st | 0.105646683 | 1.1 | 0.948040467 | 0 |
| hsa-miR-548p_st | 0.066531205 | 1.0 | 0.843745461 | 0 |
| hsa-miR-548q_st | −0.764227588 | 1.7 | 0.000146092 | 1 |
| hsa-miR-548s_st | −0.02215727 | 1.0 | 0.582525502 | 0 |
| hsa-miR-548t_st | −0.045938372 | 1.0 | 0.397777564 | 0 |
| hsa-miR-548u_st | 0.335583711 | 1.3 | 0.202550776 | 0 |
| hsa-miR-638_st | −0.010692313 | 1.0 | 0.870008646 | 0 |
| hsa-miR-548w_st | 0.062890236 | 1.0 | 0.754125712 | 0 |
| hsa-miR-3119_st | −0.120941951 | 1.1 | 0.202890168 | 0 |
| hsa-miR-549_st | −0.019785312 | 1.0 | 0.948777667 | 0 |
| hsa-miR-205_st | 0.919503386 | 1.9 | 0.126603166 | 0 |
| hsa-miR-550-star_st | 0.423733934 | 1.3 | 0.700524508 | 0 |
| hsa-miR-551a_st | −0.026394791 | 1.0 | 0.632299916 | 0 |
| hsa-miR-93_st | 1.316454658 | 2.5 | 0.001256201 | 1 |
| hsa-miR-551b-star_st | 0.022968016 | 1.0 | 0.776835489 | 0 |
| hsa-let-7d-star_st | 0.725060868 | 1.7 | 0.028345162 | 1 |
| hsa-miR-553_st | 0.228656256 | 1.2 | 0.107976485 | 0 |
| hsa-miR-554_st | −0.079907143 | 1.1 | 0.261310822 | 0 |
| hsa-miR-555_st | −0.183975779 | 1.1 | 0.043510403 | 1 |
| hsa-miR-556-3p_st | −0.165194459 | 1.1 | 0.12275829 | 0 |
| hsa-miR-556-5p_st | 0.134116363 | 1.1 | 0.787222646 | 0 |
| hsa-miR-557_st | −0.554496973 | 1.5 | 0.003307682 | 1 |
| hsa-miR-558_st | 0.201336596 | 1.1 | 0.212711162 | 0 |
| hsa-miR-559_st | 0.071711274 | 1.1 | 0.911630553 | 0 |
| hsa-miR-561_st | 0.047188353 | 1.0 | 0.965398954 | 0 |
| hsa-miR-562_st | 0.054915353 | 1.0 | 0.816102928 | 0 |
| hsa-miR-563_st | 0.009966974 | 1.0 | 0.810157422 | 0 |
| hsa-miR-361-5p_st | 1.108905457 | 2.2 | 5.96082E−06 | 1 |
| hsa-miR-566_st | −0.290040905 | 1.2 | 0.060673372 | 0 |
| hsa-miR-567_st | −0.206447885 | 1.2 | 0.138576754 | 0 |
| hsa-miR-568_st | 0.000723722 | 1.0 | 0.785135731 | 0 |
| hsa-miR-29c_st | 1.328300194 | 2.5 | 3.39711E−05 | 1 |
| hsa-miR-570_st | 0.214552405 | 1.2 | 0.550796157 | 0 |
| hsa-miR-571_st | −0.061229473 | 1.0 | 0.567493691 | 0 |
| hsa-miR-572_st | −0.547334433 | 1.5 | 0.003043097 | 1 |
| hsa-miR-573_st | 0.038677237 | 1.0 | 0.928813658 | 0 |
| hsa-miR-132_st | 1.632328536 | 3.1 | 1.12141E−06 | 1 |
| hsa-miR-574-5p_st | −0.047786855 | 1.0 | 0.605645966 | 0 |
| hsa-let-7d_st | 0.815898536 | 1.8 | 0.000824037 | 1 |
| hsa-miR-576-3p_st | −0.143684496 | 1.1 | 0.175134581 | 0 |
| hsa-miR-576-5p_st | −0.055506895 | 1.0 | 0.525334138 | 0 |
| hsa-miR-577_st | −0.03556996 | 1.0 | 0.494231971 | 0 |
| hsa-miR-578_st | 0.469196689 | 1.4 | 0.000245009 | 1 |
| hsa-miR-579_st | 0.126170739 | 1.1 | 0.757062969 | 0 |
| hsa-miR-580_st | −0.026764192 | 1.0 | 0.58110593 | 0 |
| hsa-miR-191_st | 1.170520227 | 2.3 | 0.000375032 | 1 |
| hsa-miR-582-3p_st | −0.062689881 | 1.0 | 0.615008094 | 0 |
| hsa-miR-582-5p_st | −0.090612263 | 1.1 | 0.221774204 | 0 |
| hsa-miR-1915_st | −0.022341375 | 1.0 | 0.790524125 | 0 |
| hsa-let-7c_st | 0.803808067 | 1.7 | 0.001218364 | 1 |
| hsa-miR-585_st | 0.097070232 | 1.1 | 0.451375948 | 0 |
| hsa-miR-586_st | 0.03814739 | 1.0 | 0.694978787 | 0 |
| hsa-miR-324-5p_st | 1.213321942 | 2.3 | 0.000822739 | 1 |
| hsa-miR-588_st | −0.096705702 | 1.1 | 0.434699598 | 0 |
| hsa-miR-589_st | 0.205412041 | 1.2 | 0.836859756 | 0 |
| hsa-miR-589-star_st | −0.15318523 | 1.1 | 0.073778148 | 0 |
| hsa-miR-188-5p_st | −0.686412511 | 1.6 | 2.00965E−05 | 1 |
| hsa-miR-103_st | 1.179605862 | 2.3 | 0.000220273 | 1 |
| hsa-miR-591_st | −0.11283514 | 1.1 | 0.318421891 | 0 |
| hsa-miR-592_st | 0.287219487 | 1.2 | 0.117340504 | 0 |
| hsa-miR-593_st | −0.297131666 | 1.2 | 0.090455411 | 0 |
| hsa-miR-23b_st | 1.005976608 | 2.0 | 2.55494E−05 | 1 |
| hsa-miR-595_st | −0.302923138 | 1.2 | 0.066277532 | 0 |
| hsa-miR-99a_st | 0.881711459 | 1.8 | 0.001028468 | 1 |
| hsa-miR-15b_st | 1.119501934 | 2.2 | 0.006089066 | 1 |
| hsa-miR-135b_st | 1.134920075 | 2.2 | 0.00053113 | 1 |
| hsa-miR-599_st | 0.116299041 | 1.1 | 0.721569135 | 0 |
| hsa-miR-600_st | −0.054526051 | 1.0 | 0.589350164 | 0 |
| hsa-miR-107_st | 1.227964029 | 2.3 | 0.000270999 | 1 |
| hsa-miR-602_st | −0.499235292 | 1.4 | 0.003014943 | 1 |
| hsa-miR-603_st | 0.134129003 | 1.1 | 0.595338944 | 0 |
| hsa-miR-3065-5p_st | 1.343878918 | 2.5 | 1.41378E−05 | 1 |
| hsa-miR-605_st | −0.024938167 | 1.0 | 0.963725922 | 0 |
| hsa-miR-24_st | 1.299778717 | 2.5 | 1.84637E−06 | 1 |
| hsa-miR-607_st | 0.214153071 | 1.2 | 0.342950759 | 0 |
| hsa-miR-608_st | −0.483908277 | 1.4 | 0.008742334 | 1 |
| hsa-miR-609_st | 0.320165562 | 1.2 | 0.060331202 | 0 |
| hsa-miR-3196_st | 0.005111657 | 1.0 | 0.890518504 | 0 |
| hsa-miR-26b_st | 1.307409876 | 2.5 | 0.000523609 | 1 |
| hsa-miR-28-5p_st | 1.180096914 | 2.3 | 0.00093076 | 1 |
| hsa-miR-613_st | −0.056855887 | 1.0 | 0.267636275 | 0 |
| hsa-miR-338-3p_st | 1.301544434 | 2.5 | 0.000633288 | 1 |
| hsa-miR-615-3p_st | −0.258953941 | 1.2 | 0.474989926 | 0 |
| hsa-miR-454_st | 1.648358843 | 3.1 | 0.003708464 | 1 |
| hsa-miR-616_st | −0.05808354 | 1.0 | 0.892545983 | 0 |
| hsa-miR-151-5p_st | 1.200716494 | 2.3 | 0.000138505 | 1 |
| hsa-miR-617_st | −0.447012056 | 1.4 | 0.117541577 | 0 |
| hsa-miR-128_st | 1.316192726 | 2.5 | 0.000766319 | 1 |
| hsa-miR-619_st | −0.096210988 | 1.1 | 0.16600817 | 0 |
| hsa-miR-620_st | −0.173763378 | 1.1 | 0.118756965 | 0 |
| hsa-miR-621_st | 0.245006559 | 1.2 | 0.095601607 | 0 |
| hsa-let-7b_st | 0.901603763 | 1.9 | 0.002372164 | 1 |
| hsa-miR-623_st | −0.563171167 | 1.5 | 0.005458933 | 1 |
| hsa-miR-624_st | 0.20648621 | 1.2 | 0.200800858 | 0 |
| hsa-miR-624-star_st | 0.092852362 | 1.1 | 0.497235964 | 0 |
| hsa-miR-183_st | 1.153214093 | 2.2 | 0.021280779 | 1 |
| hsa-miR-30b_st | 1.323660775 | 2.5 | 0.000115837 | 1 |
| hsa-miR-626_st | 0.083991901 | 1.1 | 0.773036467 | 0 |
| hsa-miR-627_st | 0.115102085 | 1.1 | 0.98200278 | 0 |
| hsa-miR-628-3p_st | −0.597876998 | 1.5 | 0.018097305 | 1 |
| hsa-miR-31-star_st | 2.95516693 | 7.8 | 8.40808E−06 | 1 |
| hsa-miR-218-1-star_st | 0.019223529 | 1.0 | 0.854012682 | 0 |
| hsa-miR-629-star_st | 0.42813735 | 1.3 | 0.039800462 | 1 |
| hsa-miR-630_st | −0.793740227 | 1.7 | 0.011695073 | 1 |
| hsa-miR-631_st | −0.697127546 | 1.6 | 0.003865987 | 1 |
| hsa-miR-632_st | 0.10249186 | 1.1 | 0.662502801 | 0 |
| hsa-miR-633_st | 0.111376809 | 1.1 | 0.684042887 | 0 |
| hsa-miR-634_st | 0.054779708 | 1.0 | 0.701961312 | 0 |
| hsa-miR-635_st | −0.123472743 | 1.1 | 0.541749232 | 0 |
| hsa-miR-203_st | 1.380152897 | 2.6 | 0.001372286 | 1 |
| hsa-miR-331-3p_st | 0.843945301 | 1.8 | 0.012373089 | 1 |
| hsa-miR-125b_st | 0.930522281 | 1.9 | 3.23532E−05 | 1 |
| hsa-let-7i_st | 1.304026353 | 2.5 | 4.73346E−05 | 1 |
| hsa-miR-640_st | −0.147969894 | 1.1 | 0.443934792 | 0 |
| hsa-miR-641_st | 0.102753081 | 1.1 | 0.915582667 | 0 |
| hsa-miR-642_st | 0.126270613 | 1.1 | 0.519612819 | 0 |
| hsa-miR-29b_st | 1.763846347 | 3.4 | 1.40667E−06 | 1 |
| hsa-miR-122_st | −0.479354476 | 1.4 | 0.010846614 | 1 |
| hsa-miR-645_st | −0.121847591 | 1.1 | 0.14825713 | 0 |
| hsa-miR-646_st | 0.124012357 | 1.1 | 0.319852173 | 0 |
| hsa-miR-647_st | −0.062675481 | 1.0 | 0.778020524 | 0 |
| hsa-miR-648_st | −0.629157048 | 1.5 | 0.000416072 | 1 |
| hsa-miR-649_st | 0.103239826 | 1.1 | 0.565659407 | 0 |
| hsa-miR-182_st | 1.483418105 | 2.8 | 0.003984442 | 1 |
| hsa-miR-651_st | −0.028427575 | 1.0 | 0.497954662 | 0 |
| hsa-miR-652_st | −0.101245497 | 1.1 | 0.380732905 | 0 |
| hsa-miR-653_st | 0.178894233 | 1.1 | 0.660059889 | 0 |
| hsa-miR-654-3p_st | 0.286686636 | 1.2 | 0.607544215 | 0 |
| hsa-miR-374b_st | 1.643381641 | 3.1 | 0.000157002 | 1 |
| hsa-miR-130a_st | 1.414936386 | 2.7 | 4.65391E−05 | 1 |
| hsa-miR-656_st | −0.041729916 | 1.0 | 0.467218433 | 0 |
| hsa-miR-657_st | −0.018447866 | 1.0 | 0.781685938 | 0 |
| hsa-miR-658_st | −0.578638762 | 1.5 | 0.004086297 | 1 |
| hsa-miR-99b_st | 1.230967815 | 2.3 | 2.67522E−07 | 1 |
| hsa-miR-660_st | 1.065262832 | 2.1 | 0.015165758 | 1 |
| hsa-miR-661_st | −0.546815735 | 1.5 | 0.006490085 | 1 |
| hsa-miR-30e-star_st | 1.052544393 | 2.1 | 0.002485259 | 1 |
| hsa-miR-663_st | −0.214772664 | 1.2 | 0.160677462 | 0 |
| hsa-miR-663b_st | −0.585193882 | 1.5 | 0.012885321 | 1 |
| hsa-miR-664_st | 0.428380051 | 1.3 | 0.145493286 | 0 |
| hsa-miR-664-star_st | −0.342841978 | 1.3 | 0.305667842 | 0 |
| hsa-miR-30d_st | 1.119681573 | 2.2 | 0.000617852 | 1 |
| hsa-miR-668_st | −0.351093124 | 1.3 | 0.181456934 | 0 |
| hsa-miR-670_st | −0.238296944 | 1.2 | 0.139767008 | 0 |
| hsa-miR-671-3p_st | 0.183507426 | 1.1 | 0.114141268 | 0 |
| hsa-miR-671-5p_st | −0.636090027 | 1.6 | 2.96268E−05 | 1 |
| hsa-miR-30a-star_st | 1.096210685 | 2.1 | 0.000119159 | 1 |
| hsa-miR-675-star_st | −0.192759172 | 1.1 | 0.306860739 | 0 |
| hsa-miR-148b_st | 1.030726446 | 2.0 | 0.026559243 | 1 |
| hsa-miR-708_st | 0.18954795 | 1.1 | 0.385007072 | 0 |
| hsa-miR-708-star_st | 0.002207619 | 1.0 | 0.891128538 | 0 |
| hsa-miR-221-star_st | 2.996227 | 8.0 | 3.75772E−09 | 1 |
| hsa-let-7e_st | 1.158251204 | 2.2 | 6.93945E−05 | 1 |
| hsa-miR-7-1-star_st | 1.089007502 | 2.1 | 0.00228216 | 1 |
| hsa-miR-27b_st | 1.527125861 | 2.9 | 6.73838E−06 | 1 |
| hsa-miR-34a_st | 1.920605124 | 3.8 | 9.12061E−08 | 1 |
| hsa-miR-744_st | −0.49943743 | 1.4 | 0.001120325 | 1 |
| hsa-miR-744-star_st | 0.319311407 | 1.2 | 0.092304286 | 0 |
| hsa-miR-92b_st | 1.241521176 | 2.4 | 0.00717976 | 1 |
| hsa-miR-759_st | 0.22332995 | 1.2 | 0.266934081 | 0 |
| hsa-miR-760_st | −0.561549654 | 1.5 | 0.002974681 | 1 |
| hsa-miR-92a_st | 1.466980282 | 2.8 | 0.000179428 | 1 |
| hsa-miR-335_st | 1.470918517 | 2.8 | 0.000224463 | 1 |
| hsa-miR-96_st | 1.583315263 | 3.0 | 0.005022445 | 1 |
| hsa-miR-95_st | 1.223567046 | 2.3 | 0.000158676 | 1 |
| hsa-miR-766_st | −0.278158091 | 1.2 | 0.056005892 | 0 |
| hsa-miR-767-3p_st | 0.033397634 | 1.0 | 0.989784253 | 0 |
| hsa-miR-767-5p_st | −0.042732302 | 1.0 | 0.531562479 | 0 |
| hsa-miR-98_st | 1.235453156 | 2.4 | 0.001678843 | 1 |
| hsa-miR-769-5p_st | 0.404750144 | 1.3 | 0.008723972 | 1 |
| hsa-miR-770-5p_st | −0.107249208 | 1.1 | 0.821855114 | 0 |
| hsa-miR-802_st | −0.000886966 | 1.0 | 0.70437267 | 0 |
| hsa-miR-34b-star_st | 2.005268347 | 4.0 | 3.63387E−09 | 1 |
| hsa-miR-581_st | −0.015680568 | 1.0 | 0.737019566 | 0 |
| hsa-miR-181d_st | 1.479502434 | 2.8 | 1.37945E−08 | 1 |
| hsa-miR-875-5p_st | 0.029255834 | 1.0 | 0.638005997 | 0 |
| hsa-miR-876-3p_st | −0.146072123 | 1.1 | 0.179879274 | 0 |
| hsa-miR-876-5p_st | −0.04955472 | 1.0 | 0.500219566 | 0 |
| hsa-miR-151-3p_st | 1.659396316 | 3.2 | 8.85256E−06 | 1 |
| hsa-miR-877-star_st | −0.314539104 | 1.2 | 0.15218137 | 0 |
| hsa-miR-885-3p_st | −0.569096773 | 1.5 | 0.003529943 | 1 |
| hsa-miR-200c_st | 1.102081595 | 2.1 | 1.6401E−05 | 1 |
| hsa-miR-429_st | 2.326589242 | 5.0 | 6.66032E−07 | 1 |
| hsa-miR-886-5p_st | 0.020342006 | 1.0 | 0.58069058 | 0 |
| hsa-miR-224_st | 1.819791189 | 3.5 | 8.6003E−09 | 1 |
| hsa-miR-888_st | 0.152772525 | 1.1 | 0.648238043 | 0 |
| hsa-miR-4321_st | −0.771553945 | 1.7 | 0.000508592 | 1 |
| hsa-miR-889_st | −0.163642042 | 1.1 | 0.139539478 | 0 |
| hsa-miR-181b_st | 1.661310236 | 3.2 | 3.29519E−09 | 1 |
| hsa-miR-891a_st | −0.001905825 | 1.0 | 0.675958956 | 0 |
| hsa-miR-891b_st | 0.122778528 | 1.1 | 0.84602129 | 0 |
| hsa-miR-892a_st | −0.116949666 | 1.1 | 0.423227968 | 0 |
| hsa-miR-892b_st | 0.166561641 | 1.1 | 0.177432545 | 0 |
| hsa-miR-9_st | −0.054746755 | 1.0 | 0.422141884 | 0 |
| hsa-miR-222_st | 2.983301769 | 7.9 | 1.24496E−09 | 1 |
| hsa-miR-141_st | 1.72323742 | 3.3 | 8.17029E−07 | 1 |
| hsa-miR-221_st | 3.002048527 | 8.0 | 1.37416E−09 | 1 |
| hsa-miR-924_st | 0.211703889 | 1.2 | 0.111204346 | 0 |
| hsa-miR-181a_st | 1.719136859 | 3.3 | 9.38048E−07 | 1 |
| hsa-miR-181c_st | 1.769387685 | 3.4 | 1.74068E−05 | 1 |
| hsa-miR-4324_st | 1.469023191 | 2.8 | 2.62923E−06 | 1 |
| hsa-miR-125a-5p_st | 1.345227037 | 2.5 | 2.24453E−06 | 1 |
| hsa-miR-92b-star_st | −0.442572641 | 1.4 | 0.004154007 | 1 |
| hsa-miR-3195_st | −0.583939454 | 1.5 | 0.002089016 | 1 |
| hsa-miR-3137_st | −0.797016102 | 1.7 | 0.000110528 | 1 |
| hsa-miR-934_st | −0.01308772 | 1.0 | 0.949481661 | 0 |
| hsa-miR-935_st | 0.024765003 | 1.0 | 0.940017372 | 0 |
| hsa-miR-551b_st | 2.825353785 | 7.1 | 6.46443E−08 | 1 |
| hsa-miR-937_st | −0.14642889 | 1.1 | 0.573077793 | 0 |
| hsa-miR-938_st | −0.199216575 | 1.1 | 0.172445635 | 0 |
| hsa-miR-939_st | −0.580530727 | 1.5 | 0.000753121 | 1 |
| hsa-miR-7-2-star_st | −0.899816343 | 1.9 | 0.036822994 | 1 |
| hsa-miR-146b-5p_st | 4.916324482 | 30.2 | 3.73776E−13 | 1 |
| hsa-miR-941_st | −0.079960899 | 1.1 | 0.224876105 | 0 |
| hsa-miR-942_st | 0.16103157 | 1.1 | 0.939611539 | 0 |
| hsa-miR-200a_st | 2.070836736 | 4.2 | 5.32624E−05 | 1 |
| hsa-miR-944_st | 0.28470109 | 1.2 | 0.168615289 | 0 |
| hsa-miR-7_st | −1.527533332 | 2.9 | 0.024812208 | 1 |
| hsa-miR-375_st | 3.296427574 | 9.8 | 2.11039E−10 | 1 |
| hsa-miR-96-star_st | −0.03863582 | 1.0 | 0.708015916 | 0 |
| hsa-miR-424_st | 2.803832229 | 7.0 | 2.81321E−07 | 1 |
| hsa-miR-181a-2-star_st | 2.887223763 | 7.4 | 1.80909E−11 | 1 |
| hsa-miR-200b_st | 2.038934882 | 4.1 | 1.02714E−05 | 1 |
| hsa-miR-146b-3p_st | 3.798493183 | 13.9 | 9.04344E−10 | 1 |
| hsa-miR-99b-star_st | 0.225796912 | 1.2 | 0.038445447 | 1 |
| hsa-miR-9-star_st | −0.059772051 | 1.0 | 0.705017587 | 0 |

| | Log2 Diff | Fold change | T-test | |
|---|---|---|---|---|
| | (FTC vs | (FTC vs | (FTC vs | |
| Sample Name | Benign) | Benign) | Benign) | p < 0.05 |
| hsa-miR-150_st | −0.705125203 | 1.6 | 0.376736 | 0 |
| hsa-let-7a-2-star_st | 0.444703567 | 1.4 | 0.014871 | 1 |
| hsa-let-7a-star_st | −0.088495885 | 1.1 | 0.542407 | 0 |
| hsa-miR-513b_st | −0.777765295 | 1.7 | 0.314133 | 0 |
| hsa-let-7b-star_st | 0.28622064 | 1.2 | 0.510261 | 0 |
| hsa-miR-593-star_st | −0.415621434 | 1.3 | 0.485997 | 0 |
| hsa-let-7c-star_st | 0.095923767 | 1.1 | 0.71416 | 0 |
| hsa-miR-516a-5p_st | −0.436403863 | 1.4 | 0.198673 | 0 |
| hsa-miR-142-5p_st | 0.30182632 | 1.2 | 0.658574 | 0 |
| hsa-miR-650_st | 0.04478799 | 1.0 | 0.803883 | 0 |
| hsa-miR-513c_st | −0.79272027 | 1.7 | 0.13602 | 0 |
| hsa-miR-761_st | 0.272311973 | 1.2 | 0.396859 | 0 |
| hsa-let-7f-1-star_st | −0.046513066 | 1.0 | 0.798852 | 0 |
| hsa-miR-513a-5p_st | −0.258693788 | 1.2 | 0.62051 | 0 |
| hsa-miR-654-5p_st | −0.57149263 | 1.5 | 0.086647 | 0 |
| hsa-let-7g-star_st | 0.051740906 | 1.0 | 0.72708 | 0 |
| hsa-miR-3163_st | −0.380261859 | 1.3 | 0.223572 | 0 |
| hsa-let-7i-star_st | 0.507658113 | 1.4 | 0.197572 | 0 |
| hsa-miR-3165_st | −0.648782648 | 1.6 | 0.237405 | 0 |
| hsa-miR-3194_st | −0.348397348 | 1.3 | 0.27491 | 0 |
| hsa-miR-100-star_st | −0.173286704 | 1.1 | 0.471298 | 0 |
| hsa-miR-4251_st | −0.377615035 | 1.3 | 0.284328 | 0 |
| hsa-miR-101-star_st | 0.249556218 | 1.2 | 0.112121 | 0 |
| hsa-miR-922_st | −0.295733273 | 1.2 | 0.376891 | 0 |
| hsa-miR-103-2-star_st | 0.526503588 | 1.4 | 0.030399 | 1 |
| hsa-miR-155_st | 0.337820839 | 1.3 | 0.548863 | 0 |
| hsa-miR-105_st | −0.391655476 | 1.3 | 0.01529 | 1 |
| hsa-miR-127-5p_st | −0.210908557 | 1.2 | 0.613088 | 0 |
| hsa-miR-1286_st | −0.083760738 | 1.1 | 0.817773 | 0 |
| hsa-miR-106a-star_st | −0.037976884 | 1.0 | 0.841387 | 0 |
| hsa-miR-890_st | 0.40629793 | 1.3 | 0.373653 | 0 |
| hsa-miR-3126-5p_st | −0.536833165 | 1.5 | 0.230247 | 0 |
| hsa-miR-489_st | −0.227689784 | 1.2 | 0.631372 | 0 |
| hsa-miR-10a_st | 0.504540486 | 1.4 | 0.403827 | 0 |
| hsa-miR-10a-star_st | −0.391923756 | 1.3 | 0.069901 | 0 |
| hsa-miR-10b_st | 0.95615843 | 1.9 | 0.097359 | 0 |
| hsa-miR-10b-star_st | 0.065608654 | 1.0 | 0.808163 | 0 |
| hsa-miR-1178_st | −0.191014901 | 1.1 | 0.320212 | 0 |
| hsa-miR-1179_st | 0.112417074 | 1.1 | 0.526058 | 0 |
| hsa-miR-1180_st | −0.320031753 | 1.2 | 0.045333 | 1 |
| hsa-miR-1181_st | −0.064303295 | 1.0 | 0.805207 | 0 |
| hsa-miR-3138_st | −0.514667783 | 1.4 | 0.256269 | 0 |
| hsa-miR-220c_st | −0.158010245 | 1.1 | 0.591023 | 0 |
| hsa-miR-510_st | −0.506521703 | 1.4 | 0.240887 | 0 |
| hsa-miR-1185_st | −0.116364427 | 1.1 | 0.492826 | 0 |
| hsa-miR-1193_st | −0.311693551 | 1.2 | 0.020153 | 1 |
| hsa-miR-1197_st | −0.034176522 | 1.0 | 0.748052 | 0 |
| hsa-miR-1200_st | −0.168222557 | 1.1 | 0.25252 | 0 |
| hsa-miR-1201_st | 1.346322281 | 2.5 | 0.000722 | 1 |
| hsa-miR-1202_st | −0.333126988 | 1.3 | 0.11136 | 0 |
| hsa-miR-30c-1-star_st | −0.304027673 | 1.2 | 0.449863 | 0 |
| hsa-miR-1204_st | −0.528670243 | 1.4 | 0.03777 | 1 |
| hsa-miR-1205_st | −0.150470946 | 1.1 | 0.365049 | 0 |
| hsa-miR-1206_st | 0.011492385 | 1.0 | 0.931796 | 0 |
| hsa-miR-1207-3p_st | −0.249260077 | 1.2 | 0.228909 | 0 |
| hsa-miR-1207-5p_st | −0.139350179 | 1.1 | 0.167506 | 0 |
| hsa-miR-1208_st | −0.266966819 | 1.2 | 0.174928 | 0 |
| hsa-miR-622_st | −0.184818836 | 1.1 | 0.517665 | 0 |
| hsa-miR-1224-3p_st | 0.375517913 | 1.3 | 0.045058 | 1 |
| hsa-miR-2116_st | −0.507855103 | 1.4 | 0.086096 | 0 |
| hsa-miR-1225-3p_st | −0.2387609 | 1.2 | 0.160723 | 0 |
| hsa-miR-1225-5p_st | −0.253987797 | 1.2 | 0.030509 | 1 |
| hsa-miR-1226_st | −0.19661027 | 1.1 | 0.483823 | 0 |
| hsa-miR-30b-star_st | −0.499633441 | 1.4 | 0.1344 | 0 |
| hsa-miR-1227_st | −0.243625314 | 1.2 | 0.231206 | 0 |
| hsa-miR-1295_st | −0.436149087 | 1.4 | 0.158667 | 0 |
| hsa-miR-1228-star_st | −0.165010503 | 1.1 | 0.258944 | 0 |
| hsa-miR-20b_st | 1.372613841 | 2.6 | 0.000586 | 1 |
| hsa-miR-122-star_st | −0.092535904 | 1.1 | 0.564342 | 0 |
| hsa-miR-1231_st | −0.235406452 | 1.2 | 0.109654 | 0 |
| hsa-miR-1233_st | −0.298218185 | 1.2 | 0.177965 | 0 |
| hsa-miR-524-5p_st | 0.20482492 | 1.2 | 0.331235 | 0 |
| hsa-miR-1236_st | 0.021262206 | 1.0 | 0.90298 | 0 |
| hsa-miR-873_st | −0.257309376 | 1.2 | 0.464234 | 0 |
| hsa-miR-1238_st | −0.302667989 | 1.2 | 0.073741 | 0 |
| hsa-miR-124_st | −0.128693872 | 1.1 | 0.653969 | 0 |
| hsa-miR-1243_st | −0.074140994 | 1.1 | 0.624874 | 0 |
| hsa-miR-1244_st | 0.241688021 | 1.2 | 0.548686 | 0 |
| hsa-miR-1245_st | −0.317269112 | 1.2 | 0.035948 | 1 |
| hsa-miR-3122_st | −0.474409619 | 1.4 | 0.002429 | 1 |
| hsa-miR-134_st | −0.334717066 | 1.3 | 0.169914 | 0 |
| hsa-miR-1248_st | 0.524117052 | 1.4 | 0.372505 | 0 |
| hsa-miR-146a_st | 0.000814103 | 1.0 | 0.998876 | 0 |
| hsa-miR-124-star_st | −0.273369935 | 1.2 | 0.073895 | 0 |
| hsa-miR-1250_st | −0.380906207 | 1.3 | 0.064371 | 0 |
| hsa-miR-764_st | −0.292379222 | 1.2 | 0.354669 | 0 |
| hsa-miR-1252_st | −0.001167698 | 1.0 | 0.994297 | 0 |
| hsa-miR-223_st | 0.509957872 | 1.4 | 0.465572 | 0 |
| hsa-miR-34c-3p_st | −0.338884405 | 1.3 | 0.305562 | 0 |
| hsa-miR-1255a_st | 0.356106778 | 1.3 | 0.269823 | 0 |
| hsa-miR-1255b_st | 0.183244498 | 1.1 | 0.690477 | 0 |
| hsa-miR-1256_st | −0.083690389 | 1.1 | 0.623034 | 0 |
| hsa-miR-1257_st | 0.010421012 | 1.0 | 0.959291 | 0 |
| hsa-miR-199b-5p_st | −0.702876123 | 1.6 | 0.456912 | 0 |
| hsa-miR-1259_st | −0.0867272 | 1.1 | 0.635978 | 0 |
| hsa-miR-492_st | −0.328908498 | 1.3 | 0.094757 | 0 |
| hsa-miR-3132_st | −0.020588389 | 1.0 | 0.933656 | 0 |
| hsa-miR-516b_st | −0.305671919 | 1.2 | 0.439099 | 0 |
| hsa-miR-4294_st | −0.39687903 | 1.3 | 0.307969 | 0 |
| hsa-miR-125b-2-star_st | 0.252725504 | 1.2 | 0.530309 | 0 |
| hsa-miR-126_st | 0.58986271 | 1.5 | 0.022983 | 1 |
| hsa-miR-198_st | −0.227017752 | 1.2 | 0.45234 | 0 |
| hsa-miR-3191_st | −0.110464634 | 1.1 | 0.750375 | 0 |
| hsa-miR-1261_st | −0.402076009 | 1.3 | 0.332579 | 0 |
| hsa-miR-1262_st | −0.173430033 | 1.1 | 0.423837 | 0 |
| hsa-miR-1263_st | 0.034996778 | 1.0 | 0.858567 | 0 |
| hsa-miR-1264_st | −0.152946217 | 1.1 | 0.40921 | 0 |
| hsa-miR-1265_st | 0.227693074 | 1.2 | 0.234864 | 0 |
| hsa-miR-2114-star_st | −0.232898825 | 1.2 | 0.598992 | 0 |
| hsa-miR-1267_st | −0.283716499 | 1.2 | 0.028521 | 1 |
| hsa-miR-1268_st | −0.265494198 | 1.2 | 0.02751 | 1 |
| hsa-miR-1269_st | −0.067539498 | 1.0 | 0.75183 | 0 |
| hsa-miR-509-5p_st | −0.452332726 | 1.4 | 0.152492 | 0 |
| hsa-miR-1270_st | −0.159396552 | 1.1 | 0.500581 | 0 |
| hsa-miR-1271_st | 0.274148026 | 1.2 | 0.277666 | 0 |
| hsa-miR-4303_st | −0.372052404 | 1.3 | 0.226101 | 0 |
| hsa-miR-1273_st | −0.127787814 | 1.1 | 0.683312 | 0 |
| hsa-miR-583_st | 0.362006726 | 1.3 | 0.229503 | 0 |
| hsa-miR-1273d_st | 0.004524162 | 1.0 | 0.981568 | 0 |
| hsa-miR-127-3p_st | 0.371937468 | 1.3 | 0.564117 | 0 |
| hsa-miR-522-star_st | −0.204907492 | 1.2 | 0.273233 | 0 |
| hsa-miR-214-star_st | −0.15408747 | 1.1 | 0.768303 | 0 |
| hsa-miR-184_st | −0.097760115 | 1.1 | 0.773863 | 0 |
| hsa-miR-614_st | −0.158201907 | 1.1 | 0.691126 | 0 |
| hsa-miR-526b_st | 0.073159625 | 1.1 | 0.715194 | 0 |
| hsa-miR-1277_st | −0.015773675 | 1.0 | 0.912449 | 0 |
| hsa-miR-1278_st | 0.126417987 | 1.1 | 0.445945 | 0 |
| hsa-miR-1279_st | −0.181762902 | 1.1 | 0.306589 | 0 |
| hsa-miR-409-3p_st | −0.215378355 | 1.2 | 0.615119 | 0 |
| hsa-miR-199a-5p_st | −0.153644046 | 1.1 | 0.830237 | 0 |
| hsa-miR-1281_st | −0.416877219 | 1.3 | 0.051909 | 0 |
| hsa-miR-1282_st | −0.114126036 | 1.1 | 0.69452 | 0 |
| hsa-miR-33b-star_st | −0.311696271 | 1.2 | 0.064741 | 0 |
| hsa-miR-1284_st | 0.029751854 | 1.0 | 0.898056 | 0 |
| hsa-miR-1253_st | −0.207949898 | 1.2 | 0.313972 | 0 |
| hsa-miR-584_st | −0.414372924 | 1.3 | 0.136767 | 0 |
| hsa-miR-1287_st | 0.114178651 | 1.1 | 0.523653 | 0 |
| hsa-miR-15a_st | 1.807026676 | 3.5 | 2.43E−06 | 1 |
| hsa-miR-1289_st | −0.075351136 | 1.1 | 0.774966 | 0 |
| hsa-miR-1290_st | 0.154585932 | 1.1 | 0.612329 | 0 |
| hsa-miR-2117_st | −0.4678768 | 1.4 | 0.021404 | 1 |
| hsa-miR-1292_st | −0.473551934 | 1.4 | 0.006783 | 1 |
| hsa-miR-1293_st | −0.337402691 | 1.3 | 0.021723 | 1 |
| hsa-miR-129-3p_st | −0.063749598 | 1.0 | 0.756925 | 0 |
| hsa-miR-520d-5p_st | 0.117665596 | 1.1 | 0.57154 | 0 |
| hsa-miR-526a_st | 0.200592349 | 1.1 | 0.339392 | 0 |
| hsa-miR-129-5p_st | −0.145688178 | 1.1 | 0.358345 | 0 |
| hsa-miR-1254_st | −0.53796665 | 1.5 | 0.077597 | 0 |
| hsa-miR-1297_st | −0.23189402 | 1.2 | 0.169286 | 0 |
| hsa-miR-1298_st | −0.075777234 | 1.1 | 0.755096 | 0 |
| hsa-miR-1299_st | −0.249617214 | 1.2 | 0.598079 | 0 |
| hsa-miR-129-star_st | −0.052633967 | 1.0 | 0.802861 | 0 |
| hsa-miR-1301_st | −0.159684896 | 1.1 | 0.125708 | 0 |
| hsa-miR-19b_st | 1.451379133 | 2.7 | 2.02E−05 | 1 |
| hsa-miR-1303_st | 0.030203033 | 1.0 | 0.863646 | 0 |
| hsa-miR-1304_st | 0.008870957 | 1.0 | 0.979471 | 0 |
| hsa-miR-1305_st | −0.172064495 | 1.1 | 0.340802 | 0 |
| hsa-miR-550_st | −0.080479496 | 1.1 | 0.745654 | 0 |
| hsa-miR-1307_st | −0.28766034 | 1.2 | 0.078944 | 0 |
| hsa-miR-1308_st | 0.221936442 | 1.2 | 0.37792 | 0 |
| hsa-miR-4291_st | 0.771241259 | 1.7 | 0.007207 | 1 |
| hsa-miR-130a-star_st | 0.043246681 | 1.0 | 0.799175 | 0 |
| hsa-miR-130b_st | 0.306714001 | 1.2 | 0.359874 | 0 |
| hsa-miR-130b-star_st | 0.185794413 | 1.1 | 0.476992 | 0 |
| hsa-miR-921_st | −0.390782102 | 1.3 | 0.234679 | 0 |
| hsa-miR-520a-5p_st | −0.173424195 | 1.1 | 0.388112 | 0 |
| hsa-miR-711_st | −0.133322628 | 1.1 | 0.519927 | 0 |
| hsa-miR-1323_st | −0.312477393 | 1.2 | 0.116213 | 0 |
| hsa-miR-1324_st | −0.220231116 | 1.2 | 0.318216 | 0 |
| hsa-miR-490-3p_st | −0.25827324 | 1.2 | 0.17654 | 0 |
| hsa-miR-718_st | −0.461697063 | 1.4 | 0.026313 | 1 |
| hsa-miR-133b_st | −0.07985615 | 1.1 | 0.933955 | 0 |
| hsa-miR-431-star_st | −0.408662942 | 1.3 | 0.010748 | 1 |
| hsa-miR-552_st | −0.329687403 | 1.3 | 0.277994 | 0 |
| hsa-miR-28-3p_st | 0.880574763 | 1.8 | 0.072737 | 0 |
| hsa-miR-4265_st | −0.163278138 | 1.1 | 0.37954 | 0 |
| hsa-miR-643_st | −0.29977365 | 1.2 | 0.20083 | 0 |
| hsa-miR-136_st | −0.12132186 | 1.1 | 0.399267 | 0 |
| hsa-miR-136-star_st | −0.152368751 | 1.1 | 0.343058 | 0 |
| hsa-miR-137_st | −0.102684521 | 1.1 | 0.412208 | 0 |
| hsa-miR-138_st | −0.605925477 | 1.5 | 0.317351 | 0 |
| hsa-miR-138-1-star_st | −0.240945764 | 1.2 | 0.508704 | 0 |
| hsa-miR-138-2-star_st | 0.141983277 | 1.1 | 0.396209 | 0 |
| hsa-miR-139-3p_st | −0.131716305 | 1.1 | 0.54494 | 0 |
| hsa-miR-139-5p_st | −0.268734608 | 1.2 | 0.63242 | 0 |
| hsa-miR-140-3p_st | 0.197465852 | 1.1 | 0.503398 | 0 |
| hsa-miR-140-5p_st | 0.826693764 | 1.8 | 0.048991 | 1 |
| hsa-miR-381_st | 0.168334871 | 1.1 | 0.515567 | 0 |
| hsa-miR-141-star_st | 0.043126751 | 1.0 | 0.788021 | 0 |
| hsa-miR-17-star_st | 0.722778333 | 1.7 | 0.01285 | 1 |
| hsa-miR-4300_st | −0.08322793 | 1.1 | 0.670807 | 0 |
| hsa-miR-143_st | 0.387768109 | 1.3 | 0.302712 | 0 |
| hsa-miR-143-star_st | 0.382176259 | 1.3 | 0.355454 | 0 |
| hsa-miR-144_st | −0.097199106 | 1.1 | 0.578235 | 0 |
| hsa-miR-3198_st | −0.3358716 | 1.3 | 0.12322 | 0 |
| hsa-miR-145_st | −0.058239856 | 1.0 | 0.899497 | 0 |
| hsa-miR-145-star_st | 0.441691277 | 1.4 | 0.387584 | 0 |
| hsa-miR-1468_st | −0.089941631 | 1.1 | 0.636448 | 0 |
| hsa-miR-1469_st | −0.089331544 | 1.1 | 0.61786 | 0 |
| hsa-miR-1973_st | −0.34235067 | 1.3 | 0.42235 | 0 |
| hsa-miR-3154_st | −0.315751671 | 1.2 | 0.081374 | 0 |
| hsa-miR-148a-star_st | 0.318720552 | 1.2 | 0.409371 | 0 |
| hsa-miR-92a-1-star_st | −0.370453425 | 1.3 | 0.016293 | 1 |
| hsa-miR-147_st | −0.344570036 | 1.3 | 0.098676 | 0 |
| hsa-miR-194-star_st | −0.129448023 | 1.1 | 0.576788 | 0 |
| hsa-miR-1471_st | −0.651064872 | 1.6 | 0.038347 | 1 |
| hsa-miR-147b_st | −0.189832086 | 1.1 | 0.178474 | 0 |
| hsa-miR-148a_st | 1.029407673 | 2.0 | 0.087036 | 0 |
| hsa-miR-4299_st | −0.292511539 | 1.2 | 0.598714 | 0 |
| hsa-miR-135a-star_st | −0.56750887 | 1.5 | 0.021055 | 1 |
| hsa-miR-612_st | −0.45504285 | 1.4 | 0.043613 | 1 |
| hsa-miR-1321_st | −0.156177112 | 1.1 | 0.448367 | 0 |
| hsa-miR-149-star_st | −0.110476034 | 1.1 | 0.429908 | 0 |
| hsa-miR-604_st | −0.434634009 | 1.4 | 0.310819 | 0 |
| hsa-miR-150-star_st | −0.206514116 | 1.2 | 0.11544 | 0 |
| hsa-miR-125b-1-star_st | −0.375972697 | 1.3 | 0.133211 | 0 |
| hsa-miR-1183_st | −0.276599132 | 1.2 | 0.262493 | 0 |
| hsa-miR-152_st | 0.958009077 | 1.9 | 0.008709 | 1 |
| hsa-miR-153_st | −0.2703596 | 1.2 | 0.23357 | 0 |
| hsa-miR-1537_st | −0.11479074 | 1.1 | 0.421789 | 0 |
| hsa-miR-1538_st | −0.466069543 | 1.4 | 0.046073 | 1 |
| hsa-miR-1539_st | −0.04250634 | 1.0 | 0.76586 | 0 |
| hsa-miR-154_st | −0.304429124 | 1.2 | 0.422016 | 0 |
| hsa-miR-154-star_st | −0.438637174 | 1.4 | 0.052235 | 0 |
| hsa-miR-432-star_st | −0.192388767 | 1.1 | 0.452368 | 0 |
| hsa-miR-155-star_st | −0.026380858 | 1.0 | 0.893388 | 0 |
| hsa-miR-298_st | −0.222992341 | 1.2 | 0.342505 | 0 |
| hsa-miR-15a-star_st | −0.352711367 | 1.3 | 0.147192 | 0 |
| hsa-miR-2110_st | −0.237125015 | 1.2 | 0.419191 | 0 |
| hsa-miR-15b-star_st | −0.154561729 | 1.1 | 0.270312 | 0 |
| hsa-miR-1266_st | −0.36065288 | 1.3 | 0.088305 | 0 |
| hsa-miR-16-1-star_st | 0.041106483 | 1.0 | 0.733885 | 0 |
| hsa-miR-16-2-star_st | 0.088135904 | 1.1 | 0.772933 | 0 |
| hsa-miR-490-5p_st | −0.218094027 | 1.2 | 0.391697 | 0 |
| hsa-miR-19a_st | 1.168009101 | 2.2 | 0.006414 | 1 |
| hsa-miR-3192_st | −0.222235789 | 1.2 | 0.276692 | 0 |
| hsa-miR-3155_st | −0.324061844 | 1.3 | 0.14995 | 0 |
| hsa-miR-187-star_st | −0.422413005 | 1.3 | 0.009889 | 1 |
| hsa-miR-16_st | 1.052228302 | 2.1 | 0.000129 | 1 |
| hsa-miR-936_st | −0.116698523 | 1.1 | 0.443455 | 0 |
| hsa-miR-181c-star_st | −0.128046196 | 1.1 | 0.666287 | 0 |
| hsa-miR-379_st | 0.103915381 | 1.1 | 0.787539 | 0 |
| hsa-miR-370_st | −0.483340487 | 1.4 | 0.01525 | 1 |
| hsa-miR-541_st | −0.189869149 | 1.1 | 0.252191 | 0 |
| hsa-miR-518c-star_st | −0.031576082 | 1.0 | 0.855207 | 0 |
| hsa-miR-487b_st | 1.292259999 | 2.4 | 0.001643 | 1 |
| hsa-miR-182-star_st | 0.000264806 | 1.0 | 0.998582 | 0 |
| hsa-miR-3121_st | −0.254354348 | 1.2 | 0.595464 | 0 |
| hsa-miR-3190-5p_st | −0.192943008 | 1.1 | 0.256749 | 0 |
| hsa-miR-1203_st | −0.122799264 | 1.1 | 0.552063 | 0 |
| hsa-miR-185_st | 0.41879612 | 1.3 | 0.106428 | 0 |
| hsa-miR-185-star_st | −0.311398307 | 1.2 | 0.15727 | 0 |
| hsa-miR-4317_st | −0.186656579 | 1.1 | 0.425507 | 0 |
| hsa-miR-186-star_st | −0.309879283 | 1.2 | 0.015556 | 1 |
| hsa-miR-187_st | 0.317732657 | 1.2 | 0.428303 | 0 |
| hsa-miR-4257_st | −0.344345613 | 1.3 | 0.106052 | 0 |
| hsa-miR-188-3p_st | 0.081307044 | 1.1 | 0.741929 | 0 |
| hsa-miR-1184_st | −0.134432676 | 1.1 | 0.43379 | 0 |
| hsa-miR-18a_st | 0.544780248 | 1.5 | 0.028378 | 1 |
| hsa-miR-18a-star_st | −0.107360646 | 1.1 | 0.632394 | 0 |
| hsa-miR-3140_st | −0.114113368 | 1.1 | 0.616387 | 0 |
| hsa-miR-18b-star_st | −0.296717513 | 1.2 | 0.062203 | 0 |
| hsa-miR-190_st | −0.413658722 | 1.3 | 0.031424 | 1 |
| hsa-miR-601_st | −0.612690056 | 1.5 | 0.032494 | 1 |
| hsa-miR-1909_st | −0.180427622 | 1.1 | 0.217014 | 0 |
| hsa-miR-342-5p_st | 0.150492646 | 1.1 | 0.717562 | 0 |
| hsa-miR-575_st | −0.241176456 | 1.2 | 0.441334 | 0 |
| hsa-miR-885-5p_st | 0.18303072 | 1.1 | 0.586245 | 0 |
| hsa-miR-300_st | 0.056407833 | 1.0 | 0.768857 | 0 |
| hsa-miR-1911_st | −0.194707081 | 1.1 | 0.229225 | 0 |
| hsa-miR-1911-star_st | 0.13933715 | 1.1 | 0.464454 | 0 |
| hsa-miR-1912_st | −0.170970394 | 1.1 | 0.277155 | 0 |
| hsa-miR-4266_st | −0.104850116 | 1.1 | 0.54924 | 0 |
| hsa-miR-1914_st | 0.070008609 | 1.0 | 0.696735 | 0 |
| hsa-miR-3190-3p_st | −0.226461966 | 1.2 | 0.255129 | 0 |
| hsa-miR-618_st | −0.094787371 | 1.1 | 0.539664 | 0 |
| hsa-miR-1276_st | −0.281174902 | 1.2 | 0.279381 | 0 |
| hsa-miR-191-star_st | −0.319250976 | 1.2 | 0.166035 | 0 |
| hsa-miR-1258_st | −0.326707862 | 1.3 | 0.145904 | 0 |
| hsa-miR-192-star_st | 0.089090105 | 1.1 | 0.516265 | 0 |
| hsa-miR-193a-3p_st | 0.414470066 | 1.3 | 0.373314 | 0 |
| hsa-miR-193a-5p_st | −0.302295791 | 1.2 | 0.116096 | 0 |
| hsa-miR-3157_st | −0.300939044 | 1.2 | 0.186472 | 0 |
| hsa-miR-193b-star_st | −0.305529357 | 1.2 | 0.053539 | 0 |
| hsa-miR-194_st | 1.36731178 | 2.6 | 0.012301 | 1 |
| hsa-miR-4307_st | 0.043969744 | 1.0 | 0.862372 | 0 |
| hsa-miR-195_st | 0.742369169 | 1.7 | 0.090203 | 0 |
| hsa-miR-610_st | −0.114539588 | 1.1 | 0.682918 | 0 |
| hsa-miR-196a_st | −0.339068156 | 1.3 | 0.137665 | 0 |
| hsa-miR-196a-star_st | −0.118876622 | 1.1 | 0.816708 | 0 |
| hsa-miR-196b_st | −0.361576561 | 1.3 | 0.121272 | 0 |
| hsa-miR-196b-star_st | 0.115703873 | 1.1 | 0.667523 | 0 |
| hsa-let-7f-2-star_st | 0.051677749 | 1.0 | 0.706143 | 0 |
| hsa-miR-1972_st | −0.046908963 | 1.0 | 0.839737 | 0 |
| hsa-miR-3184_st | −0.297481284 | 1.2 | 0.242275 | 0 |
| hsa-miR-523-star_st | −0.11871377 | 1.1 | 0.637685 | 0 |
| hsa-miR-1976_st | 0.200978515 | 1.1 | 0.437262 | 0 |
| hsa-miR-625-star_st | −0.2984619 | 1.2 | 0.077316 | 0 |
| hsa-miR-587_st | −0.379036452 | 1.3 | 0.18636 | 0 |
| hsa-miR-199a-3p_st | −0.086977511 | 1.1 | 0.893946 | 0 |
| hsa-miR-1910_st | −0.280374108 | 1.2 | 0.061371 | 0 |
| hsa-miR-199b-3p_st | −0.085186759 | 1.1 | 0.90189 | 0 |
| hsa-miR-639_st | −0.633117298 | 1.6 | 0.004681 | 1 |
| hsa-miR-1909-star_st | −0.04954437 | 1.0 | 0.81209 | 0 |
| hsa-miR-19a-star_st | 0.023358024 | 1.0 | 0.910746 | 0 |
| hsa-miR-29c-star_st | 1.155865738 | 2.2 | 0.001798 | 1 |
| hsa-miR-940_st | −0.454260244 | 1.4 | 0.036272 | 1 |
| hsa-miR-19b-2-star_st | −0.101114755 | 1.1 | 0.440535 | 0 |
| hsa-miR-4269_st | 0.014021399 | 1.0 | 0.929756 | 0 |
| hsa-miR-200a-star_st | −0.274118588 | 1.2 | 0.347631 | 0 |
| hsa-miR-1182_st | −0.261863445 | 1.2 | 0.09322 | 0 |
| hsa-miR-518f-star_st | 0.205221025 | 1.2 | 0.22068 | 0 |
| hsa-miR-92a-2-star_st | −0.145559488 | 1.1 | 0.520364 | 0 |
| hsa-miR-200c-star_st | −0.061475953 | 1.0 | 0.757001 | 0 |
| hsa-miR-202_st | −0.495516659 | 1.4 | 0.032312 | 1 |
| hsa-miR-202-star_st | −0.113639918 | 1.1 | 0.574156 | 0 |
| hsa-miR-1914-star_st | −0.377571705 | 1.3 | 0.102503 | 0 |
| hsa-miR-204_st | −0.368184866 | 1.3 | 0.696686 | 0 |
| hsa-miR-665_st | −0.042755449 | 1.0 | 0.798664 | 0 |
| hsa-miR-2052_st | −0.148341662 | 1.1 | 0.398067 | 0 |
| hsa-miR-2053_st | −0.066783818 | 1.0 | 0.681403 | 0 |
| hsa-miR-2054_st | −0.199844712 | 1.1 | 0.344208 | 0 |
| hsa-miR-205-star_st | 0.027775609 | 1.0 | 0.889702 | 0 |
| hsa-miR-206_st | 0.306819243 | 1.2 | 0.693583 | 0 |
| hsa-miR-208a_st | −0.158871129 | 1.1 | 0.348101 | 0 |
| hsa-miR-208b_st | −0.016238865 | 1.0 | 0.911071 | 0 |
| hsa-miR-18b_st | 0.159834763 | 1.1 | 0.358119 | 0 |
| hsa-miR-20a-star_st | 0.029034482 | 1.0 | 0.904565 | 0 |
| hsa-miR-3181_st | −0.569427582 | 1.5 | 0.011033 | 1 |
| hsa-miR-615-5p_st | −0.471935172 | 1.4 | 0.056438 | 0 |
| hsa-miR-4259_st | −0.45310523 | 1.4 | 0.020785 | 1 |
| hsa-miR-210_st | −0.335932209 | 1.3 | 0.008511 | 1 |
| hsa-miR-211_st | −0.508584719 | 1.4 | 0.033641 | 1 |
| hsa-miR-887_st | −0.181749273 | 1.1 | 0.164489 | 0 |
| hsa-miR-2113_st | −0.023304227 | 1.0 | 0.915606 | 0 |
| hsa-miR-2114_st | −0.119928599 | 1.1 | 0.656448 | 0 |
| hsa-miR-943_st | −0.369406796 | 1.3 | 0.136634 | 0 |
| hsa-miR-2115_st | −0.142254256 | 1.1 | 0.535572 | 0 |
| hsa-miR-2115-star_st | −0.263281006 | 1.2 | 0.449397 | 0 |
| hsa-miR-183-star_st | −0.127199067 | 1.1 | 0.47531 | 0 |
| hsa-miR-2116-star_st | −0.235248272 | 1.2 | 0.096805 | 0 |
| hsa-miR-920_st | −0.223642303 | 1.2 | 0.20911 | 0 |
| hsa-miR-212_st | 0.445188139 | 1.4 | 0.078512 | 0 |
| hsa-miR-214_st | 0.141515557 | 1.1 | 0.571069 | 0 |
| hsa-miR-1825_st | −0.193738466 | 1.1 | 0.406357 | 0 |
| hsa-miR-215_st | 0.479236378 | 1.4 | 0.240999 | 0 |
| hsa-miR-216a_st | 0.035703908 | 1.0 | 0.82587 | 0 |
| hsa-miR-195-star_st | −0.030258076 | 1.0 | 0.911065 | 0 |
| hsa-miR-217_st | 0.013053857 | 1.0 | 0.900443 | 0 |
| hsa-miR-23a-star_st | −0.15292494 | 1.1 | 0.29645 | 0 |
| hsa-miR-25-star_st | −0.209561617 | 1.2 | 0.153408 | 0 |
| hsa-miR-218-2-star_st | −0.540426686 | 1.5 | 0.002063 | 1 |
| hsa-miR-1273c_st | −0.050599807 | 1.0 | 0.788984 | 0 |
| hsa-miR-219-2-3p_st | −0.118445865 | 1.1 | 0.51403 | 0 |
| hsa-miR-637_st | −0.399574676 | 1.3 | 0.011645 | 1 |
| hsa-miR-21-star_st | 0.285052116 | 1.2 | 0.493057 | 0 |
| hsa-miR-22_st | 0.897792429 | 1.9 | 0.022714 | 1 |
| hsa-miR-220a_st | −0.022107469 | 1.0 | 0.865493 | 0 |
| hsa-miR-220b_st | 0.109162523 | 1.1 | 0.442214 | 0 |
| hsa-miR-219-1-3p_st | −0.054584771 | 1.0 | 0.787164 | 0 |
| hsa-miR-4316_st | −0.111686867 | 1.1 | 0.502461 | 0 |
| hsa-miR-877_st | −0.407612628 | 1.3 | 0.041149 | 1 |
| hsa-miR-636_st | −0.409250291 | 1.3 | 0.036468 | 1 |
| hsa-miR-3177_st | −0.379106948 | 1.3 | 0.035684 | 1 |
| hsa-miR-1249_st | −0.037996395 | 1.0 | 0.900178 | 0 |
| hsa-miR-223-star_st | 0.105811485 | 1.1 | 0.558056 | 0 |
| hsa-miR-1224-5p_st | −0.209188781 | 1.2 | 0.082302 | 0 |
| hsa-miR-564_st | −0.032944601 | 1.0 | 0.839399 | 0 |
| hsa-miR-2276_st | −0.217574237 | 1.2 | 0.398712 | 0 |
| hsa-miR-2277_st | −0.227538142 | 1.2 | 0.321315 | 0 |
| hsa-miR-2278_st | −0.266525885 | 1.2 | 0.180176 | 0 |
| hsa-miR-22-star_st | 1.398061863 | 2.6 | 0.007064 | 1 |
| hsa-miR-2355_st | −0.113098651 | 1.1 | 0.403945 | 0 |
| hsa-miR-769-3p_st | −0.163950585 | 1.1 | 0.531021 | 0 |
| hsa-miR-1913_st | −0.087377293 | 1.1 | 0.670422 | 0 |
| hsa-miR-1237_st | −0.156005014 | 1.1 | 0.483759 | 0 |
| hsa-miR-1275_st | −0.182473605 | 1.1 | 0.245485 | 0 |
| hsa-miR-30a_st | 1.024729276 | 2.0 | 0.00251 | 1 |
| hsa-miR-20b-star_st | −0.057160051 | 1.0 | 0.842689 | 0 |
| hsa-miR-24-2-star_st | 0.586785533 | 1.5 | 0.109135 | 0 |
| hsa-miR-3173_st | −0.099329894 | 1.1 | 0.533755 | 0 |
| hsa-miR-3148_st | −0.021975634 | 1.0 | 0.917253 | 0 |
| hsa-miR-4271_st | −0.222495069 | 1.2 | 0.318823 | 0 |
| hsa-miR-659_st | −0.288319897 | 1.2 | 0.028321 | 1 |
| hsa-miR-26a-2-star_st | 0.149258411 | 1.1 | 0.390824 | 0 |
| hsa-miR-1228_st | −0.315200757 | 1.2 | 0.165415 | 0 |
| hsa-miR-26b-star_st | 0.367285802 | 1.3 | 0.207723 | 0 |
| hsa-miR-3174_st | −0.077758999 | 1.1 | 0.816929 | 0 |
| hsa-miR-27a-star_st | 0.129101911 | 1.1 | 0.681076 | 0 |
| hsa-miR-1285_st | 0.002028737 | 1.0 | 0.990615 | 0 |
| hsa-miR-483-5p_st | −0.103887481 | 1.1 | 0.48891 | 0 |
| hsa-miR-1306_st | −0.372007961 | 1.3 | 0.056927 | 0 |
| hsa-miR-431_st | 0.073637654 | 1.1 | 0.692312 | 0 |
| hsa-miR-765_st | −0.083915062 | 1.1 | 0.439635 | 0 |
| hsa-miR-2909_st | −0.044400927 | 1.0 | 0.802129 | 0 |
| hsa-miR-296-3p_st | −0.317483684 | 1.2 | 0.021269 | 1 |
| hsa-miR-296-5p_st | 0.103216593 | 1.1 | 0.630197 | 0 |
| hsa-miR-297_st | −0.09159831 | 1.1 | 0.748298 | 0 |
| hsa-miR-323b-5p_st | −0.074971997 | 1.1 | 0.731642 | 0 |
| hsa-miR-299-3p_st | −0.357201162 | 1.3 | 0.16969 | 0 |
| hsa-miR-299-5p_st | 0.113927724 | 1.1 | 0.835393 | 0 |
| hsa-miR-487a_st | 0.484682834 | 1.4 | 0.022022 | 1 |
| hsa-miR-29a-star_st | −0.201991506 | 1.2 | 0.192164 | 0 |
| hsa-miR-625_st | 0.02015697 | 1.0 | 0.897879 | 0 |
| hsa-miR-611_st | −0.047362374 | 1.0 | 0.823486 | 0 |
| hsa-miR-3186-3p_st | 0.321723361 | 1.2 | 0.081443 | 0 |
| hsa-miR-1294_st | −0.334680741 | 1.3 | 0.06991 | 0 |
| hsa-miR-3147_st | −0.355102141 | 1.3 | 0.004452 | 1 |
| hsa-miR-4285_st | −0.23332227 | 1.2 | 0.247803 | 0 |
| hsa-miR-125a-3p_st | −0.19105969 | 1.1 | 0.228396 | 0 |
| hsa-miR-301b_st | −0.190560974 | 1.1 | 0.314794 | 0 |
| hsa-miR-302a_st | −0.116046946 | 1.1 | 0.44629 | 0 |
| hsa-miR-518e-star_st | 0.164830266 | 1.1 | 0.327913 | 0 |
| hsa-miR-302b_st | −0.305863491 | 1.2 | 0.085766 | 0 |
| hsa-miR-302b-star_st | −0.40169618 | 1.3 | 0.076571 | 0 |
| hsa-miR-302c_st | 0.052955397 | 1.0 | 0.794809 | 0 |
| hsa-miR-302c-star_st | −0.21732841 | 1.2 | 0.297699 | 0 |
| hsa-miR-302d_st | −0.212558094 | 1.2 | 0.262774 | 0 |
| hsa-miR-302d-star_st | −0.152154928 | 1.1 | 0.577364 | 0 |
| hsa-miR-302e_st | −0.00543141 | 1.0 | 0.978307 | 0 |
| hsa-miR-302f_st | −0.011169252 | 1.0 | 0.953059 | 0 |
| hsa-miR-3065-3p_st | −0.01400565 | 1.0 | 0.965258 | 0 |
| hsa-miR-3130-5p_st | −0.245118257 | 1.2 | 0.344544 | 0 |
| hsa-miR-3074_st | 0.010157705 | 1.0 | 0.941458 | 0 |
| hsa-miR-1226-star_st | −0.243467573 | 1.2 | 0.07422 | 0 |
| hsa-miR-3180-5p_st | −0.434566776 | 1.4 | 0.084939 | 0 |
| hsa-miR-1229_st | 0.484187598 | 1.4 | 0.011296 | 1 |
| hsa-miR-1272_st | −0.207017763 | 1.2 | 0.278179 | 0 |
| hsa-miR-30c_st | 0.929822529 | 1.9 | 0.001828 | 1 |
| hsa-miR-675_st | −0.151068925 | 1.1 | 0.39416 | 0 |
| hsa-miR-30c-2-star_st | 0.040943193 | 1.0 | 0.884835 | 0 |
| hsa-miR-1827_st | 0.216244586 | 1.2 | 0.236704 | 0 |
| hsa-miR-30d-star_st | 0.164291124 | 1.1 | 0.485466 | 0 |
| hsa-miR-30e_st | 1.34280828 | 2.5 | 0.000813 | 1 |
| hsa-miR-1915-star_st | 0.158995183 | 1.1 | 0.445988 | 0 |
| hsa-miR-1322_st | −0.334798644 | 1.3 | 0.147333 | 0 |
| hsa-miR-3115_st | −0.021601043 | 1.0 | 0.891796 | 0 |
| hsa-miR-3116_st | 0.04836237 | 1.0 | 0.903499 | 0 |
| hsa-miR-3117_st | −0.291992768 | 1.2 | 0.221832 | 0 |
| hsa-miR-3118_st | −0.018286078 | 1.0 | 0.88778 | 0 |
| hsa-miR-519b-5p_st | 0.188257503 | 1.1 | 0.228337 | 0 |
| hsa-miR-3120_st | −0.11376235 | 1.1 | 0.659387 | 0 |
| hsa-miR-520d-3p_st | −0.016086995 | 1.0 | 0.952687 | 0 |
| hsa-miR-326_st | 0.117452534 | 1.1 | 0.370762 | 0 |
| hsa-miR-3123_st | −0.125667124 | 1.1 | 0.467766 | 0 |
| hsa-miR-3124_st | −0.136411291 | 1.1 | 0.512649 | 0 |
| hsa-miR-3125_st | 0.24415869 | 1.2 | 0.30138 | 0 |
| hsa-miR-3126-3p_st | −0.250107989 | 1.2 | 0.212594 | 0 |
| hsa-miR-1296_st | 0.038576006 | 1.0 | 0.779673 | 0 |
| hsa-miR-3127_st | −0.505747042 | 1.4 | 0.123558 | 0 |
| hsa-miR-3128_st | −0.233641718 | 1.2 | 0.204258 | 0 |
| hsa-miR-3129_st | −0.175272055 | 1.1 | 0.233191 | 0 |
| hsa-miR-3130-3p_st | −0.283506905 | 1.2 | 0.155025 | 0 |
| hsa-miR-105-star_st | −0.393116139 | 1.3 | 0.007955 | 1 |
| hsa-miR-3131_st | −0.180392821 | 1.1 | 0.159988 | 0 |
| hsa-miR-4255_st | −0.196886986 | 1.1 | 0.219423 | 0 |
| hsa-miR-3133_st | −0.319483614 | 1.2 | 0.394095 | 0 |
| hsa-miR-3134_st | −0.24635543 | 1.2 | 0.098643 | 0 |
| hsa-miR-3135_st | 0.114436769 | 1.1 | 0.601731 | 0 |
| hsa-miR-3136_st | −0.409197574 | 1.3 | 0.04849 | 1 |
| hsa-miR-19b-1-star_st | 0.197460202 | 1.1 | 0.118289 | 0 |
| hsa-miR-1247_st | −0.158265771 | 1.1 | 0.517931 | 0 |
| hsa-miR-3139_st | 0.09763863 | 1.1 | 0.386084 | 0 |
| hsa-miR-596_st | −0.079817084 | 1.1 | 0.720187 | 0 |
| hsa-miR-3141_st | −0.203679005 | 1.2 | 0.13921 | 0 |
| hsa-miR-3142_st | −0.064415331 | 1.0 | 0.622409 | 0 |
| hsa-miR-1302_st | −0.214347942 | 1.2 | 0.061457 | 0 |
| hsa-miR-3144-3p_st | −0.03153786 | 1.0 | 0.842159 | 0 |
| hsa-miR-3144-5p_st | −0.183643588 | 1.1 | 0.443838 | 0 |
| hsa-miR-3145_st | −0.363162533 | 1.3 | 0.057806 | 0 |
| hsa-miR-3146_st | −0.232589803 | 1.2 | 0.33291 | 0 |
| hsa-miR-346_st | −0.428049352 | 1.3 | 0.002429 | 1 |
| hsa-miR-4256_st | −0.42950509 | 1.3 | 0.056375 | 0 |
| hsa-miR-3149_st | −0.041461654 | 1.0 | 0.841517 | 0 |
| hsa-miR-3150_st | −0.382473593 | 1.3 | 0.043291 | 1 |
| hsa-miR-3151_st | −0.240622638 | 1.2 | 0.086257 | 0 |
| hsa-miR-1470_st | −0.252384195 | 1.2 | 0.11675 | 0 |
| hsa-miR-3153_st | −0.286363041 | 1.2 | 0.1578 | 0 |
| hsa-miR-514_st | −0.081668439 | 1.1 | 0.399305 | 0 |
| hsa-miR-3175_st | 0.14356799 | 1.1 | 0.189593 | 0 |
| hsa-miR-3156_st | 0.023212504 | 1.0 | 0.87113 | 0 |
| hsa-miR-504_st | 0.11005777 | 1.1 | 0.635307 | 0 |
| hsa-miR-3158_st | −0.124572837 | 1.1 | 0.662755 | 0 |
| hsa-miR-3159_st | −0.023777996 | 1.0 | 0.910479 | 0 |
| hsa-miR-3160_st | −0.171857638 | 1.1 | 0.326015 | 0 |
| hsa-miR-3161_st | 0.414150406 | 1.3 | 0.075652 | 0 |
| hsa-miR-3162_st | −0.197060809 | 1.1 | 0.242988 | 0 |
| hsa-miR-1234_st | −0.320843263 | 1.2 | 0.180046 | 0 |
| hsa-miR-3164_st | −0.379852892 | 1.3 | 0.254876 | 0 |
| hsa-miR-516b-star_st | 0.095433654 | 1.1 | 0.568313 | 0 |
| hsa-miR-3166_st | −0.081743397 | 1.1 | 0.559271 | 0 |
| hsa-miR-4327_st | −0.35434949 | 1.3 | 0.004595 | 1 |
| hsa-miR-3168_st | 0.014122963 | 1.0 | 0.937651 | 0 |
| hsa-miR-3169_st | −0.261355705 | 1.2 | 0.172964 | 0 |
| hsa-miR-3170_st | −0.041081585 | 1.0 | 0.880919 | 0 |
| hsa-miR-3171_st | −0.088803953 | 1.1 | 0.520105 | 0 |
| hsa-miR-3172_st | 0.198219006 | 1.1 | 0.398746 | 0 |
| hsa-miR-374b-star_st | 0.27582399 | 1.2 | 0.2106 | 0 |
| hsa-miR-34c-5p_st | 0.006784123 | 1.0 | 0.95611 | 0 |
| hsa-miR-758_st | 0.025012445 | 1.0 | 0.954295 | 0 |
| hsa-miR-3176_st | −0.171286239 | 1.1 | 0.45875 | 0 |
| hsa-miR-606_st | 0.033641561 | 1.0 | 0.793768 | 0 |
| hsa-miR-3178_st | −0.176082124 | 1.1 | 0.157413 | 0 |
| hsa-miR-3179_st | −0.217618667 | 1.2 | 0.34018 | 0 |
| hsa-miR-3180-3p_st | −0.214070669 | 1.2 | 0.244807 | 0 |
| hsa-miR-597_st | 0.067309683 | 1.0 | 0.685759 | 0 |
| hsa-miR-148b-star_st | 0.212870465 | 1.2 | 0.259304 | 0 |
| hsa-miR-3182_st | −0.049737974 | 1.0 | 0.844819 | 0 |
| hsa-miR-3183_st | −0.294270331 | 1.2 | 0.125399 | 0 |
| hsa-miR-144-star_st | 0.113165889 | 1.1 | 0.588758 | 0 |
| hsa-miR-3185_st | −0.24583669 | 1.2 | 0.102708 | 0 |
| hsa-miR-4270_st | −0.227001765 | 1.2 | 0.047192 | 1 |
| hsa-miR-3186-5p_st | −0.161680845 | 1.1 | 0.416834 | 0 |
| hsa-miR-3187_st | −0.310333812 | 1.2 | 0.041588 | 1 |
| hsa-miR-3188_st | −0.26162614 | 1.2 | 0.054989 | 0 |
| hsa-miR-3189_st | −0.308532372 | 1.2 | 0.165767 | 0 |
| hsa-miR-548b-3p_st | −0.202028411 | 1.2 | 0.447298 | 0 |
| hsa-miR-99a-star_st | 0.008591644 | 1.0 | 0.985091 | 0 |
| hsa-miR-376a-star_st | −0.262366127 | 1.2 | 0.175589 | 0 |
| hsa-miR-216b_st | 0.109811539 | 1.1 | 0.475646 | 0 |
| hsa-miR-3193_st | −0.1455925 | 1.1 | 0.494757 | 0 |
| hsa-miR-548v_st | −0.127431689 | 1.1 | 0.310566 | 0 |
| hsa-miR-26a-1-star_st | −0.172882256 | 1.1 | 0.12082 | 0 |
| hsa-miR-1291_st | 0.033813753 | 1.0 | 0.923764 | 0 |
| hsa-miR-3197_st | −0.2752107 | 1.2 | 0.173261 | 0 |
| hsa-miR-629_st | 0.322102816 | 1.3 | 0.14937 | 0 |
| hsa-miR-3199_st | −0.086750107 | 1.1 | 0.77707 | 0 |
| hsa-miR-190b_st | 0.037973005 | 1.0 | 0.812507 | 0 |
| hsa-miR-32_st | 0.054203066 | 1.0 | 0.793567 | 0 |
| hsa-miR-3200_st | 0.370776003 | 1.3 | 0.26748 | 0 |
| hsa-miR-3201_st | −0.224802742 | 1.2 | 0.134516 | 0 |
| hsa-miR-3202_st | −0.037381252 | 1.0 | 0.868966 | 0 |
| hsa-miR-320a_st | 0.088642076 | 1.1 | 0.580585 | 0 |
| hsa-miR-4289_st | 0.353834194 | 1.3 | 0.217778 | 0 |
| hsa-miR-655_st | 0.098646879 | 1.1 | 0.511802 | 0 |
| hsa-miR-320b_st | 0.190618529 | 1.1 | 0.414674 | 0 |
| hsa-miR-219-5p_st | 0.138005098 | 1.1 | 0.57633 | 0 |
| hsa-miR-323-3p_st | −0.118105522 | 1.1 | 0.480847 | 0 |
| hsa-miR-323-5p_st | −0.281204668 | 1.2 | 0.210072 | 0 |
| hsa-miR-323b-3p_st | −0.199954115 | 1.1 | 0.294963 | 0 |
| hsa-miR-103-as_st | 0.327763771 | 1.3 | 0.042037 | 1 |
| hsa-miR-424-star_st | −0.079178446 | 1.1 | 0.708366 | 0 |
| hsa-miR-33a-star_st | 0.621673223 | 1.5 | 0.08119 | 0 |
| hsa-miR-325_st | −0.25410997 | 1.2 | 0.321 | 0 |
| hsa-miR-590-3p_st | −0.250220762 | 1.2 | 0.051087 | 0 |
| hsa-miR-328_st | 0.47942705 | 1.4 | 0.388897 | 0 |
| hsa-miR-329_st | 0.402623233 | 1.3 | 0.33579 | 0 |
| hsa-miR-32-star_st | −0.144735608 | 1.1 | 0.398804 | 0 |
| hsa-miR-330-3p_st | 0.296420533 | 1.2 | 0.319573 | 0 |
| hsa-miR-330-5p_st | −0.358556796 | 1.3 | 0.101513 | 0 |
| hsa-miR-146a-star_st | 0.132936281 | 1.1 | 0.436562 | 0 |
| hsa-miR-331-5p_st | 0.214925835 | 1.2 | 0.293243 | 0 |
| hsa-miR-3167_st | −0.380514101 | 1.3 | 0.011104 | 1 |
| hsa-miR-335-star_st | −0.113347924 | 1.1 | 0.667181 | 0 |
| hsa-miR-337-3p_st | −0.299917588 | 1.2 | 0.241301 | 0 |
| hsa-miR-337-5p_st | 0.098607674 | 1.1 | 0.53292 | 0 |
| hsa-miR-421_st | 0.981238385 | 2.0 | 0.000564 | 1 |
| hsa-miR-338-5p_st | 0.250102197 | 1.2 | 0.349619 | 0 |
| hsa-miR-339-3p_st | 0.215123252 | 1.2 | 0.207802 | 0 |
| hsa-miR-548a-5p_st | 0.173615523 | 1.1 | 0.219936 | 0 |
| hsa-miR-33a_st | −0.183834199 | 1.1 | 0.194999 | 0 |
| hsa-miR-590-5p_st | 0.429675725 | 1.3 | 0.049476 | 1 |
| hsa-miR-33b_st | 0.006619555 | 1.0 | 0.959539 | 0 |
| hsa-miR-616-star_st | 0.140218818 | 1.1 | 0.535721 | 0 |
| hsa-miR-569_st | −0.343456236 | 1.3 | 0.024866 | 1 |
| hsa-miR-340-star_st | 0.050611491 | 1.0 | 0.818365 | 0 |
| hsa-miR-342-3p_st | 0.74806875 | 1.7 | 0.117205 | 0 |
| hsa-miR-135b-star_st | −0.229794648 | 1.2 | 0.370114 | 0 |
| hsa-miR-345_st | 0.650838824 | 1.6 | 0.306431 | 0 |
| hsa-miR-1288_st | −0.35239905 | 1.3 | 0.031557 | 1 |
| hsa-miR-135a_st | −0.255753253 | 1.2 | 0.206675 | 0 |
| hsa-miR-34a-star_st | 0.54743006 | 1.5 | 0.247337 | 0 |
| hsa-miR-34b_st | 0.226778338 | 1.2 | 0.487582 | 0 |
| hsa-miR-4288_st | 0.168978062 | 1.1 | 0.452641 | 0 |
| hsa-miR-222-star_st | 0.455608482 | 1.4 | 0.331792 | 0 |
| hsa-miR-933_st | 0.661721296 | 1.6 | 0.043657 | 1 |
| hsa-miR-361-3p_st | 0.362386662 | 1.3 | 0.321141 | 0 |
| hsa-miR-224-star_st | 0.349123832 | 1.3 | 0.278402 | 0 |
| hsa-miR-362-3p_st | 0.846074367 | 1.8 | 0.054201 | 0 |
| hsa-miR-362-5p_st | 0.730868032 | 1.7 | 0.068527 | 0 |
| hsa-miR-363_st | 0.902059941 | 1.9 | 0.19095 | 0 |
| hsa-miR-363-star_st | −0.243662075 | 1.2 | 0.260103 | 0 |
| hsa-miR-1979_st | 0.210717114 | 1.2 | 0.319255 | 0 |
| hsa-miR-365-star_st | −0.250262759 | 1.2 | 0.062003 | 0 |
| hsa-miR-548x_st | −0.222452328 | 1.2 | 0.103741 | 0 |
| hsa-miR-367-star_st | −0.009449646 | 1.0 | 0.960316 | 0 |
| hsa-miR-369-3p_st | −0.137924493 | 1.1 | 0.493441 | 0 |
| hsa-miR-369-5p_st | 0.413624974 | 1.3 | 0.275731 | 0 |
| hsa-miR-548l_st | −0.462858659 | 1.4 | 0.010697 | 1 |
| hsa-miR-371-3p_st | −0.314238581 | 1.2 | 0.032169 | 1 |
| hsa-miR-371-5p_st | −0.329699988 | 1.3 | 0.012149 | 1 |
| hsa-miR-372_st | −0.449063987 | 1.4 | 0.064932 | 0 |
| hsa-miR-373_st | −0.001210024 | 1.0 | 0.995358 | 0 |
| hsa-miR-373-star_st | −0.241266808 | 1.2 | 0.146947 | 0 |
| hsa-miR-374a_st | 1.012265854 | 2.0 | 0.063571 | 0 |
| hsa-miR-374a-star_st | 0.319878832 | 1.2 | 0.063397 | 0 |
| hsa-miR-500_st | 0.234083858 | 1.2 | 0.520834 | 0 |
| hsa-miR-320c_st | 0.347070909 | 1.3 | 0.20656 | 0 |
| hsa-miR-875-3p_st | −0.294945917 | 1.2 | 0.01005 | 1 |
| hsa-miR-376a_st | 0.20239319 | 1.2 | 0.628111 | 0 |
| hsa-miR-29b-1-star_st | 1.039006533 | 2.1 | 0.001431 | 1 |
| hsa-miR-376b_st | 0.069831205 | 1.0 | 0.830814 | 0 |
| hsa-miR-376c_st | 0.215460011 | 1.2 | 0.73194 | 0 |
| hsa-miR-377_st | −0.254715874 | 1.2 | 0.453781 | 0 |
| hsa-miR-377-star_st | −0.090396435 | 1.1 | 0.721982 | 0 |
| hsa-miR-378_st | 0.190166057 | 1.1 | 0.754105 | 0 |
| hsa-miR-378b_st | −0.075184996 | 1.1 | 0.639191 | 0 |
| hsa-miR-378c_st | 0.393349144 | 1.3 | 0.358202 | 0 |
| hsa-miR-378-star_st | 0.619597439 | 1.5 | 0.132812 | 0 |
| hsa-miR-662_st | 0.644523647 | 1.6 | 0.006265 | 1 |
| hsa-miR-379-star_st | 0.052880072 | 1.0 | 0.758413 | 0 |
| hsa-miR-380_st | −0.132147415 | 1.1 | 0.40427 | 0 |
| hsa-miR-380-star_st | −0.080819037 | 1.1 | 0.559834 | 0 |
| hsa-miR-1274b_st | 0.692029878 | 1.6 | 0.008033 | 1 |
| hsa-miR-382_st | −0.079664111 | 1.1 | 0.866517 | 0 |
| hsa-miR-383_st | −0.19512361 | 1.1 | 0.352773 | 0 |
| hsa-miR-384_st | −0.02150677 | 1.0 | 0.903645 | 0 |
| hsa-miR-126-star_st | −0.078586982 | 1.1 | 0.638543 | 0 |
| hsa-miR-409-5p_st | 0.280141855 | 1.2 | 0.284478 | 0 |
| hsa-miR-410_st | 0.060231608 | 1.0 | 0.748033 | 0 |
| hsa-miR-411_st | −0.009065099 | 1.0 | 0.971273 | 0 |
| hsa-miR-411-star_st | 0.054650659 | 1.0 | 0.833063 | 0 |
| hsa-miR-412_st | −0.349362245 | 1.3 | 0.075651 | 0 |
| hsa-miR-3143_st | −0.309343561 | 1.2 | 0.053479 | 0 |
| hsa-miR-422a_st | 0.480802988 | 1.4 | 0.228163 | 0 |
| hsa-miR-628-5p_st | −0.010807438 | 1.0 | 0.956414 | 0 |
| hsa-miR-423-5p_st | −0.158291648 | 1.1 | 0.144523 | 0 |
| hsa-miR-27b-star_st | 1.144934747 | 2.2 | 0.000137 | 1 |
| hsa-miR-499-5p_st | 0.537593512 | 1.5 | 0.061637 | 0 |
| hsa-miR-425_st | 1.152232207 | 2.2 | 0.021107 | 1 |
| hsa-miR-193b_st | 0.777218989 | 1.7 | 0.188445 | 0 |
| hsa-miR-4252_st | −0.237928146 | 1.2 | 0.037826 | 1 |
| hsa-miR-4253_st | −0.07441884 | 1.1 | 0.624666 | 0 |
| hsa-miR-4254_st | 0.341356072 | 1.3 | 0.241972 | 0 |
| hsa-miR-324-3p_st | 0.159767742 | 1.1 | 0.5027 | 0 |
| hsa-miR-4329_st | 0.122056788 | 1.1 | 0.521979 | 0 |
| hsa-miR-1251_st | 0.49059833 | 1.4 | 0.292208 | 0 |
| hsa-miR-4258_st | −0.486496757 | 1.4 | 0.057511 | 0 |
| hsa-miR-1274a_st | 1.454236267 | 2.7 | 0.004247 | 1 |
| hsa-miR-425-star_st | 0.121147214 | 1.1 | 0.487547 | 0 |
| hsa-miR-4260_st | −0.308871356 | 1.2 | 0.072652 | 0 |
| hsa-miR-4261_st | −0.150753977 | 1.1 | 0.407443 | 0 |
| hsa-miR-4262_st | −0.284692167 | 1.2 | 0.047952 | 1 |
| hsa-miR-4263_st | −0.228452731 | 1.2 | 0.109873 | 0 |
| hsa-miR-4264_st | −0.318087101 | 1.2 | 0.066803 | 0 |
| hsa-miR-1280_st | 0.419569178 | 1.3 | 0.086435 | 0 |
| hsa-miR-132-star_st | 0.626048779 | 1.5 | 0.322813 | 0 |
| hsa-miR-4267_st | −0.344890704 | 1.3 | 0.179689 | 0 |
| hsa-miR-4268_st | −0.338895549 | 1.3 | 0.10181 | 0 |
| hsa-miR-133a_st | −0.109694948 | 1.1 | 0.906966 | 0 |
| hsa-miR-888-star_st | −0.33178815 | 1.3 | 0.058608 | 0 |
| hsa-miR-1260b_st | 0.497739532 | 1.4 | 0.040396 | 1 |
| hsa-miR-4272_st | −0.07031784 | 1.0 | 0.611592 | 0 |
| hsa-miR-4273_st | −0.182591736 | 1.1 | 0.250089 | 0 |
| hsa-miR-4274_st | −0.323186967 | 1.3 | 0.225198 | 0 |
| hsa-miR-4275_st | 0.347490459 | 1.3 | 0.049573 | 1 |
| hsa-miR-4276_st | 0.01585076 | 1.0 | 0.91909 | 0 |
| hsa-miR-4277_st | −0.137692708 | 1.1 | 0.390689 | 0 |
| hsa-miR-4278_st | −0.288592884 | 1.2 | 0.217969 | 0 |
| hsa-miR-4279_st | 0.04760162 | 1.0 | 0.822954 | 0 |
| hsa-miR-4280_st | −0.18052362 | 1.1 | 0.454556 | 0 |
| hsa-miR-4281_st | −0.126614891 | 1.1 | 0.377076 | 0 |
| hsa-miR-4282_st | −0.1487339 | 1.1 | 0.234511 | 0 |
| hsa-miR-4283_st | −0.239167612 | 1.2 | 0.115145 | 0 |
| hsa-miR-4284_st | 0.254188388 | 1.2 | 0.247511 | 0 |
| hsa-miR-23b-star_st | 0.079395816 | 1.1 | 0.773269 | 0 |
| hsa-miR-574-3p_st | 0.372528145 | 1.3 | 0.222786 | 0 |
| hsa-miR-4287_st | −0.127903826 | 1.1 | 0.581739 | 0 |
| hsa-miR-320e_st | 0.618045699 | 1.5 | 0.050576 | 0 |
| hsa-miR-339-5p_st | 0.853292759 | 1.8 | 0.074822 | 0 |
| hsa-miR-505_st | 0.778134045 | 1.7 | 0.128209 | 0 |
| hsa-miR-4290_st | 0.206393143 | 1.2 | 0.351671 | 0 |
| hsa-miR-200b-star_st | −0.397547597 | 1.3 | 0.182141 | 0 |
| hsa-miR-4292_st | −0.507388258 | 1.4 | 0.031951 | 1 |
| hsa-miR-4293_st | −0.027462161 | 1.0 | 0.892797 | 0 |
| hsa-miR-1246_st | 0.414736319 | 1.3 | 0.124844 | 0 |
| hsa-miR-4295_st | 0.07492555 | 1.1 | 0.694571 | 0 |
| hsa-miR-4296_st | −0.294484043 | 1.2 | 0.094351 | 0 |
| hsa-miR-4297_st | −0.148250227 | 1.1 | 0.631538 | 0 |
| hsa-miR-4298_st | −0.083908186 | 1.1 | 0.575612 | 0 |
| hsa-miR-218_st | 1.200331744 | 2.3 | 0.024175 | 1 |
| hsa-miR-4286_st | 0.962915116 | 1.9 | 0.002423 | 1 |
| hsa-miR-4301_st | −0.248467648 | 1.2 | 0.276115 | 0 |
| hsa-miR-4302_st | −0.377015037 | 1.3 | 0.065229 | 0 |
| hsa-miR-365_st | 0.275158224 | 1.2 | 0.558456 | 0 |
| hsa-miR-4304_st | −0.05156377 | 1.0 | 0.861577 | 0 |
| hsa-miR-4305_st | −0.170667826 | 1.1 | 0.422615 | 0 |
| hsa-miR-4306_st | 0.153490115 | 1.1 | 0.455602 | 0 |
| hsa-miR-26a_st | 0.461851114 | 1.4 | 0.058941 | 0 |
| hsa-miR-4308_st | 0.24652506 | 1.2 | 0.29665 | 0 |
| hsa-miR-4309_st | −0.067603752 | 1.0 | 0.552812 | 0 |
| hsa-miR-186_st | 0.965710015 | 2.0 | 0.082184 | 0 |
| hsa-miR-4310_st | −0.67444314 | 1.6 | 0.065911 | 0 |
| hsa-miR-4311_st | −0.02217425 | 1.0 | 0.930599 | 0 |
| hsa-miR-4312_st | −0.18012942 | 1.1 | 0.524654 | 0 |
| hsa-miR-4313_st | −0.109956685 | 1.1 | 0.571521 | 0 |
| hsa-miR-4314_st | −0.408820848 | 1.3 | 0.313019 | 0 |
| hsa-miR-4315_st | −0.245040873 | 1.2 | 0.290623 | 0 |
| hsa-miR-149_st | 0.977756894 | 2.0 | 0.046381 | 1 |
| hsa-miR-106b-star_st | 0.425124322 | 1.3 | 0.22197 | 0 |
| hsa-miR-4318_st | −0.230378957 | 1.2 | 0.124319 | 0 |
| hsa-miR-4319_st | 0.079951375 | 1.1 | 0.782544 | 0 |
| hsa-miR-29b-2-star_st | 0.826032717 | 1.8 | 0.001505 | 1 |
| hsa-miR-432_st | −0.210973988 | 1.2 | 0.483967 | 0 |
| hsa-miR-4320_st | 0.121109739 | 1.1 | 0.376654 | 0 |
| hsa-miR-522_st | −0.29123895 | 1.2 | 0.04019 | 1 |
| hsa-miR-4322_st | −0.353924865 | 1.3 | 0.010836 | 1 |
| hsa-miR-4323_st | −0.137843218 | 1.1 | 0.486405 | 0 |
| hsa-miR-93-star_st | 0.902228229 | 1.9 | 0.000326 | 1 |
| hsa-miR-4325_st | 0.044234063 | 1.0 | 0.764658 | 0 |
| hsa-miR-4326_st | −0.186687342 | 1.1 | 0.402938 | 0 |
| hsa-miR-874_st | −0.275970526 | 1.2 | 0.158548 | 0 |
| hsa-miR-4328_st | −0.13912254 | 1.1 | 0.315912 | 0 |
| hsa-let-7e-star_st | 0.385254103 | 1.3 | 0.171653 | 0 |
| hsa-miR-720_st | 0.061697499 | 1.0 | 0.747938 | 0 |
| hsa-miR-433_st | −0.003926179 | 1.0 | 0.980456 | 0 |
| hsa-miR-4330_st | −0.571714053 | 1.5 | 0.05418 | 0 |
| hsa-miR-448_st | −0.121528155 | 1.1 | 0.425056 | 0 |
| hsa-miR-449a_st | 0.296450447 | 1.2 | 0.261168 | 0 |
| hsa-miR-449b_st | −0.161666907 | 1.1 | 0.34903 | 0 |
| hsa-miR-1975_st | 0.050210437 | 1.0 | 0.779874 | 0 |
| hsa-miR-449c_st | −0.635656977 | 1.6 | 0.008109 | 1 |
| hsa-miR-449c-star_st | −0.354961117 | 1.3 | 0.131696 | 0 |
| hsa-miR-1_st | 0.327935748 | 1.3 | 0.691217 | 0 |
| hsa-miR-450b-3p_st | −0.275368593 | 1.2 | 0.055715 | 0 |
| hsa-miR-450b-5p_st | −0.201914769 | 1.2 | 0.298211 | 0 |
| hsa-miR-451_st | 0.29874469 | 1.2 | 0.60144 | 0 |
| hsa-miR-452_st | 0.394662974 | 1.3 | 0.163805 | 0 |
| hsa-miR-452-star_st | −0.116804787 | 1.1 | 0.320648 | 0 |
| hsa-miR-548b-5p_st | −0.415522331 | 1.3 | 0.047547 | 1 |
| hsa-miR-454-star_st | 0.090584749 | 1.1 | 0.684641 | 0 |
| hsa-miR-455-3p_st | 0.72695679 | 1.7 | 0.157444 | 0 |
| hsa-miR-455-5p_st | 0.745611668 | 1.7 | 0.113269 | 0 |
| hsa-miR-466_st | −0.002378655 | 1.0 | 0.9908 | 0 |
| hsa-miR-483-3p_st | −0.002771205 | 1.0 | 0.989313 | 0 |
| hsa-miR-17_st | 1.004134411 | 2.0 | 0.005165 | 1 |
| hsa-miR-320d_st | 0.541005035 | 1.5 | 0.125703 | 0 |
| hsa-miR-485-3p_st | 0.38483216 | 1.3 | 0.236994 | 0 |
| hsa-miR-485-5p_st | −0.075896024 | 1.1 | 0.656094 | 0 |
| hsa-miR-486-3p_st | −0.146028046 | 1.1 | 0.547445 | 0 |
| hsa-miR-486-5p_st | −0.521624073 | 1.4 | 0.434867 | 0 |
| hsa-miR-644_st | −0.453405252 | 1.4 | 0.014361 | 1 |
| hsa-miR-3152_st | −0.597763677 | 1.5 | 0.00553 | 1 |
| hsa-miR-488_st | −0.094910267 | 1.1 | 0.556745 | 0 |
| hsa-miR-488-star_st | −0.168114905 | 1.1 | 0.239729 | 0 |
| hsa-miR-450a_st | 0.878998262 | 1.8 | 0.056476 | 0 |
| hsa-miR-20a_st | 1.357218447 | 2.6 | 0.00111 | 1 |
| hsa-miR-484_st | 0.997234163 | 2.0 | 0.022629 | 1 |
| hsa-miR-491-3p_st | −0.117083314 | 1.1 | 0.579589 | 0 |
| hsa-miR-491-5p_st | −0.256430323 | 1.2 | 0.295795 | 0 |
| hsa-miR-197_st | 0.153738709 | 1.1 | 0.747248 | 0 |
| hsa-miR-493_st | 0.246347842 | 1.2 | 0.276198 | 0 |
| hsa-miR-493-star_st | 0.432261825 | 1.3 | 0.362606 | 0 |
| hsa-miR-494_st | −0.332715091 | 1.3 | 0.342793 | 0 |
| hsa-miR-495_st | 0.644282003 | 1.6 | 0.163043 | 0 |
| hsa-miR-496_st | −0.121088859 | 1.1 | 0.415413 | 0 |
| hsa-miR-497_st | 0.688563468 | 1.6 | 0.167353 | 0 |
| hsa-miR-497-star_st | −0.190441169 | 1.1 | 0.124663 | 0 |
| hsa-miR-498_st | −0.173247862 | 1.1 | 0.331489 | 0 |
| hsa-miR-499-3p_st | 0.099334322 | 1.1 | 0.348574 | 0 |
| hsa-miR-24-1-star_st | 0.477250912 | 1.4 | 0.088102 | 0 |
| hsa-miR-340_st | 1.756826151 | 3.4 | 0.003883 | 1 |
| hsa-miR-500b_st | 0.37849067 | 1.3 | 0.098823 | 0 |
| hsa-miR-500-star_st | 0.114575453 | 1.1 | 0.450079 | 0 |
| hsa-miR-501-3p_st | −0.110373115 | 1.1 | 0.296713 | 0 |
| hsa-miR-501-5p_st | −0.033980928 | 1.0 | 0.883225 | 0 |
| hsa-miR-502-3p_st | 0.200893962 | 1.1 | 0.229183 | 0 |
| hsa-miR-502-5p_st | −0.005450855 | 1.0 | 0.974077 | 0 |
| hsa-miR-503_st | −0.044216702 | 1.0 | 0.858667 | 0 |
| hsa-miR-192_st | 1.377755715 | 2.6 | 0.022037 | 1 |
| hsa-let-7g_st | 0.794375997 | 1.7 | 0.000967 | 1 |
| hsa-miR-505-star_st | 0.178139367 | 1.1 | 0.239215 | 0 |
| hsa-miR-506_st | −0.167615067 | 1.1 | 0.381947 | 0 |
| hsa-miR-507_st | −0.086283243 | 1.1 | 0.670709 | 0 |
| hsa-miR-508-3p_st | −0.248158399 | 1.2 | 0.106166 | 0 |
| hsa-miR-508-5p_st | −0.065235692 | 1.0 | 0.721057 | 0 |
| hsa-miR-509-3-5p_st | −0.356336468 | 1.3 | 0.272238 | 0 |
| hsa-miR-509-3p_st | −0.040166287 | 1.0 | 0.850235 | 0 |
| hsa-miR-106a_st | 1.111828532 | 2.2 | 0.002902 | 1 |
| hsa-miR-106b_st | 1.521552145 | 2.9 | 7.38E−05 | 1 |
| hsa-miR-511_st | −0.125649502 | 1.1 | 0.381641 | 0 |
| hsa-miR-512-3p_st | −0.405186046 | 1.3 | 0.019878 | 1 |
| hsa-miR-512-5p_st | 0.138080761 | 1.1 | 0.517338 | 0 |
| hsa-miR-513a-3p_st | −0.071873038 | 1.1 | 0.746684 | 0 |
| hsa-miR-23a_st | 0.538403502 | 1.5 | 0.07273 | 0 |
| hsa-miR-25_st | 1.142215498 | 2.2 | 0.005119 | 1 |
| hsa-miR-598_st | 0.920358127 | 1.9 | 0.020429 | 1 |
| hsa-miR-1260_st | 0.964245489 | 2.0 | 0.004347 | 1 |
| hsa-miR-514b-3p_st | 0.069761576 | 1.0 | 0.738866 | 0 |
| hsa-miR-514b-5p_st | 0.128152492 | 1.1 | 0.331654 | 0 |
| hsa-miR-515-3p_st | −0.357850082 | 1.3 | 0.094108 | 0 |
| hsa-miR-515-5p_st | −0.130619858 | 1.1 | 0.384019 | 0 |
| hsa-miR-516a-3p_st | −0.159304588 | 1.1 | 0.390325 | 0 |
| hsa-miR-27a_st | 1.017558305 | 2.0 | 0.001115 | 1 |
| hsa-miR-100_st | 0.533302875 | 1.4 | 0.197804 | 0 |
| hsa-miR-101_st | 1.117489124 | 2.2 | 0.039229 | 1 |
| hsa-miR-517a_st | 0.109201257 | 1.1 | 0.626314 | 0 |
| hsa-miR-517b_st | 0.02554161 | 1.0 | 0.889596 | 0 |
| hsa-miR-517c_st | −0.107471381 | 1.1 | 0.507678 | 0 |
| hsa-miR-517-star_st | 0.030431926 | 1.0 | 0.889584 | 0 |
| hsa-miR-518a-3p_st | −0.203758414 | 1.2 | 0.137186 | 0 |
| hsa-miR-518a-5p_st | −0.041030535 | 1.0 | 0.859177 | 0 |
| hsa-miR-518b_st | −0.111383873 | 1.1 | 0.615247 | 0 |
| hsa-miR-518c_st | −0.030644926 | 1.0 | 0.809031 | 0 |
| hsa-miR-301a_st | 0.752189199 | 1.7 | 0.023696 | 1 |
| hsa-miR-518d-3p_st | −0.202513779 | 1.2 | 0.098765 | 0 |
| hsa-miR-518d-5p_st | 0.434373046 | 1.4 | 0.03753 | 1 |
| hsa-miR-518e_st | −0.072593815 | 1.1 | 0.681533 | 0 |
| hsa-let-7f_st | 0.68361981 | 1.6 | 0.003231 | 1 |
| hsa-miR-518f_st | −0.159974557 | 1.1 | 0.394905 | 0 |
| hsa-miR-1826_st | −0.090063164 | 1.1 | 0.56881 | 0 |
| hsa-miR-519a_st | −0.252786209 | 1.2 | 0.122184 | 0 |
| hsa-miR-519a-star_st | 0.092211681 | 1.1 | 0.68124 | 0 |
| hsa-miR-519b-3p_st | −0.114245101 | 1.1 | 0.552331 | 0 |
| hsa-miR-1283_st | −0.469487431 | 1.4 | 0.00146 | 1 |
| hsa-miR-519c-3p_st | −0.245942745 | 1.2 | 0.251864 | 0 |
| hsa-miR-519c-5p_st | 0.115575913 | 1.1 | 0.62724 | 0 |
| hsa-miR-519d_st | −0.163140403 | 1.1 | 0.252884 | 0 |
| hsa-miR-519e_st | 0.129353681 | 1.1 | 0.548914 | 0 |
| hsa-miR-519e-star_st | −0.022303865 | 1.0 | 0.904033 | 0 |
| hsa-miR-520a-3p_st | −0.117170806 | 1.1 | 0.544033 | 0 |
| hsa-miR-1908_st | −0.061572109 | 1.0 | 0.742666 | 0 |
| hsa-miR-520b_st | −0.042398359 | 1.0 | 0.757818 | 0 |
| hsa-miR-520c-3p_st | 0.12741139 | 1.1 | 0.395223 | 0 |
| hsa-miR-520c-5p_st | 0.325644192 | 1.3 | 0.144279 | 0 |
| hsa-miR-181a-star_st | 1.066648028 | 2.1 | 0.026636 | 1 |
| hsa-miR-21_st | 1.701918012 | 3.3 | 0.003731 | 1 |
| hsa-miR-520e_st | −0.169893218 | 1.1 | 0.31581 | 0 |
| hsa-miR-520f_st | 0.103649443 | 1.1 | 0.569346 | 0 |
| hsa-miR-520g_st | 0.024343564 | 1.0 | 0.920078 | 0 |
| hsa-miR-520h_st | −0.15017653 | 1.1 | 0.352571 | 0 |
| hsa-miR-521_st | −0.002991683 | 1.0 | 0.987167 | 0 |
| hsa-miR-302a-star_st | −0.411085649 | 1.3 | 0.006121 | 1 |
| hsa-miR-886-3p_st | 0.939607637 | 1.9 | 0.094772 | 0 |
| hsa-miR-523_st | −0.337970193 | 1.3 | 0.107975 | 0 |
| hsa-miR-29a_st | 1.2319824 | 2.3 | 0.000329 | 1 |
| hsa-miR-524-3p_st | −0.320499202 | 1.2 | 0.011902 | 1 |
| hsa-miR-142-3p_st | −0.438969781 | 1.4 | 0.008531 | 1 |
| hsa-miR-525-3p_st | −0.39413845 | 1.3 | 0.048847 | 1 |
| hsa-miR-525-5p_st | 0.30479135 | 1.2 | 0.129595 | 0 |
| hsa-miR-762_st | −0.083493328 | 1.1 | 0.633601 | 0 |
| hsa-miR-423-3p_st | 0.711073882 | 1.6 | 0.017649 | 1 |
| hsa-miR-449b-star_st | −0.123220301 | 1.1 | 0.577831 | 0 |
| hsa-miR-527_st | −0.026625226 | 1.0 | 0.911283 | 0 |
| hsa-miR-532-3p_st | 0.540568774 | 1.5 | 0.126344 | 0 |
| hsa-miR-532-5p_st | 0.731613762 | 1.7 | 0.025591 | 1 |
| hsa-miR-539_st | −0.018830432 | 1.0 | 0.948402 | 0 |
| hsa-let-7a_st | 0.55204636 | 1.5 | 0.020682 | 1 |
| hsa-miR-541-star_st | −0.109865546 | 1.1 | 0.460317 | 0 |
| hsa-miR-542-3p_st | 0.387574158 | 1.3 | 0.457678 | 0 |
| hsa-miR-542-5p_st | 0.284172013 | 1.2 | 0.138411 | 0 |
| hsa-miR-543_st | 0.20446825 | 1.2 | 0.528457 | 0 |
| hsa-miR-544_st | −0.202343739 | 1.2 | 0.173628 | 0 |
| hsa-miR-544b_st | −0.253850799 | 1.2 | 0.034089 | 1 |
| hsa-miR-545_st | −0.136117108 | 1.1 | 0.486843 | 0 |
| hsa-miR-545-star_st | −0.020733575 | 1.0 | 0.925047 | 0 |
| hsa-miR-548a-3p_st | −0.333045477 | 1.3 | 0.140219 | 0 |
| hsa-miR-2861_st | −0.099423093 | 1.1 | 0.56761 | 0 |
| hsa-miR-31_st | −0.109783603 | 1.1 | 0.921003 | 0 |
| hsa-miR-367_st | −0.378471209 | 1.3 | 0.063538 | 0 |
| hsa-miR-548c-3p_st | −0.186050898 | 1.1 | 0.444477 | 0 |
| hsa-miR-548c-5p_st | 0.176010745 | 1.1 | 0.593144 | 0 |
| hsa-miR-548d-3p_st | −0.223879669 | 1.2 | 0.142763 | 0 |
| hsa-miR-548d-5p_st | 0.036623603 | 1.0 | 0.885945 | 0 |
| hsa-miR-548e_st | −0.080672478 | 1.1 | 0.526272 | 0 |
| hsa-miR-548f_st | −0.048696998 | 1.0 | 0.727798 | 0 |
| hsa-miR-548g_st | −0.100754796 | 1.1 | 0.620972 | 0 |
| hsa-miR-548h_st | 0.256969314 | 1.2 | 0.210166 | 0 |
| hsa-miR-548i_st | −0.204849676 | 1.2 | 0.215507 | 0 |
| hsa-miR-548j_st | −0.268777248 | 1.2 | 0.275809 | 0 |
| hsa-miR-548k_st | −0.105556905 | 1.1 | 0.320016 | 0 |
| hsa-miR-526b-star_st | −0.390518138 | 1.3 | 0.016474 | 1 |
| hsa-miR-548m_st | −0.05771275 | 1.0 | 0.660989 | 0 |
| hsa-miR-548n_st | 0.028413019 | 1.0 | 0.810969 | 0 |
| hsa-miR-548o_st | −0.153729074 | 1.1 | 0.314199 | 0 |
| hsa-miR-548p_st | −0.194119527 | 1.1 | 0.319165 | 0 |
| hsa-miR-548q_st | −0.281681877 | 1.2 | 0.229742 | 0 |
| hsa-miR-548s_st | −0.127317026 | 1.1 | 0.404318 | 0 |
| hsa-miR-548t_st | −0.354088888 | 1.3 | 0.039372 | 1 |
| hsa-miR-548u_st | −0.172982469 | 1.1 | 0.451723 | 0 |
| hsa-miR-638_st | −0.070465295 | 1.1 | 0.709102 | 0 |
| hsa-miR-548w_st | −0.052329994 | 1.0 | 0.877859 | 0 |
| hsa-miR-3119_st | −0.418162043 | 1.3 | 0.044401 | 1 |
| hsa-miR-549_st | −0.396111481 | 1.3 | 0.007663 | 1 |
| hsa-miR-205_st | −0.184997991 | 1.1 | 0.861434 | 0 |
| hsa-miR-550-star_st | 0.575253391 | 1.5 | 0.135855 | 0 |
| hsa-miR-551a_st | −0.2814343 | 1.2 | 0.184199 | 0 |
| hsa-miR-93_st | 1.187465426 | 2.3 | 0.001441 | 1 |
| hsa-miR-551b-star_st | 0.049585931 | 1.0 | 0.792342 | 0 |
| hsa-let-7d-star_st | 0.28165121 | 1.2 | 0.596534 | 0 |
| hsa-miR-553_st | 0.007816507 | 1.0 | 0.960785 | 0 |
| hsa-miR-554_st | −0.168313693 | 1.1 | 0.165081 | 0 |
| hsa-miR-555_st | −0.22003929 | 1.2 | 0.136724 | 0 |
| hsa-miR-556-3p_st | −0.391882801 | 1.3 | 0.026404 | 1 |
| hsa-miR-556-5p_st | −0.058236078 | 1.0 | 0.723537 | 0 |
| hsa-miR-557_st | −0.390448873 | 1.3 | 0.039219 | 1 |
| hsa-miR-558_st | 0.083444317 | 1.1 | 0.58926 | 0 |
| hsa-miR-559_st | −0.11985025 | 1.1 | 0.429681 | 0 |
| hsa-miR-561_st | −0.01781633 | 1.0 | 0.915846 | 0 |
| hsa-miR-562_st | −0.040378468 | 1.0 | 0.790005 | 0 |
| hsa-miR-563_st | −0.187165458 | 1.1 | 0.280274 | 0 |
| hsa-miR-361-5p_st | 0.723844648 | 1.7 | 0.03287 | 1 |
| hsa-miR-566_st | −0.130903377 | 1.1 | 0.346761 | 0 |
| hsa-miR-567_st | −0.401702551 | 1.3 | 0.157023 | 0 |
| hsa-miR-568_st | −0.20566138 | 1.2 | 0.220733 | 0 |
| hsa-miR-29c_st | 1.326485605 | 2.5 | 0.000467 | 1 |
| hsa-miR-570_st | −0.102974732 | 1.1 | 0.404312 | 0 |
| hsa-miR-571_st | −0.306693555 | 1.2 | 0.106421 | 0 |
| hsa-miR-572_st | −0.424231993 | 1.3 | 0.003436 | 1 |
| hsa-miR-573_st | −0.085227674 | 1.1 | 0.518144 | 0 |
| hsa-miR-132_st | 1.114746429 | 2.2 | 0.07689 | 0 |
| hsa-miR-574-5p_st | 0.136408684 | 1.1 | 0.393694 | 0 |
| hsa-let-7d_st | 0.61890098 | 1.5 | 0.014918 | 1 |
| hsa-miR-576-3p_st | −0.143494699 | 1.1 | 0.515039 | 0 |
| hsa-miR-576-5p_st | −0.049197078 | 1.0 | 0.834912 | 0 |
| hsa-miR-577_st | −0.218217894 | 1.2 | 0.260749 | 0 |
| hsa-miR-578_st | −0.103644333 | 1.1 | 0.465353 | 0 |
| hsa-miR-579_st | −0.156269307 | 1.1 | 0.246716 | 0 |
| hsa-miR-580_st | −0.211660157 | 1.2 | 0.154681 | 0 |
| hsa-miR-191_st | 0.87234231 | 1.8 | 0.011941 | 1 |
| hsa-miR-582-3p_st | −0.243485005 | 1.2 | 0.073892 | 0 |
| hsa-miR-582-5p_st | −0.170330076 | 1.1 | 0.648452 | 0 |
| hsa-miR-1915_st | −0.072399572 | 1.1 | 0.702103 | 0 |
| hsa-let-7c_st | 0.517563473 | 1.4 | 0.061755 | 0 |
| hsa-miR-585_st | −0.211688096 | 1.2 | 0.159342 | 0 |
| hsa-miR-586_st | −0.14429517 | 1.1 | 0.360386 | 0 |
| hsa-miR-324-5p_st | 0.988319838 | 2.0 | 0.012885 | 1 |
| hsa-miR-588_st | −0.167131422 | 1.1 | 0.332784 | 0 |
| hsa-miR-589_st | 0.218479222 | 1.2 | 0.252838 | 0 |
| hsa-miR-589-star_st | −0.00641305 | 1.0 | 0.973753 | 0 |
| hsa-miR-188-5p_st | −0.296936153 | 1.2 | 0.219384 | 0 |
| hsa-miR-103_st | 1.03523233 | 2.0 | 0.001823 | 1 |
| hsa-miR-591_st | −0.143378031 | 1.1 | 0.419704 | 0 |
| hsa-miR-592_st | 0.130916791 | 1.1 | 0.591192 | 0 |
| hsa-miR-593_st | −0.186610547 | 1.1 | 0.243335 | 0 |
| hsa-miR-23b_st | 0.721602597 | 1.6 | 0.010104 | 1 |
| hsa-miR-595_st | −0.31487096 | 1.2 | 0.041805 | 1 |
| hsa-miR-99a_st | 0.495327316 | 1.4 | 0.300085 | 0 |
| hsa-miR-15b_st | 0.947101333 | 1.9 | 0.061733 | 0 |
| hsa-miR-135b_st | 0.546460614 | 1.5 | 0.155533 | 0 |
| hsa-miR-599_st | −0.030449943 | 1.0 | 0.882528 | 0 |
| hsa-miR-600_st | 0.13457278 | 1.1 | 0.618444 | 0 |
| hsa-miR-107_st | 1.141220602 | 2.2 | 0.000543 | 1 |
| hsa-miR-602_st | −0.187890095 | 1.1 | 0.172407 | 0 |
| hsa-miR-603_st | 0.006426969 | 1.0 | 0.95885 | 0 |
| hsa-miR-3065-5p_st | 0.373447558 | 1.3 | 0.474244 | 0 |
| hsa-miR-605_st | −0.321490282 | 1.2 | 0.166105 | 0 |
| hsa-miR-24_st | 0.87897293 | 1.8 | 0.005853 | 1 |
| hsa-miR-607_st | −0.017611727 | 1.0 | 0.891842 | 0 |
| hsa-miR-608_st | −0.279737283 | 1.2 | 0.059528 | 0 |
| hsa-miR-609_st | 0.064318178 | 1.0 | 0.648074 | 0 |
| hsa-miR-3196_st | −0.071836039 | 1.1 | 0.704866 | 0 |
| hsa-miR-26b_st | 1.226846823 | 2.3 | 0.000933 | 1 |
| hsa-miR-28-5p_st | 1.073394755 | 2.1 | 0.000182 | 1 |
| hsa-miR-613_st | −0.209291608 | 1.2 | 0.15186 | 0 |
| hsa-miR-338-3p_st | 0.957359286 | 1.9 | 0.097284 | 0 |
| hsa-miR-615-3p_st | −0.096389676 | 1.1 | 0.632522 | 0 |
| hsa-miR-454_st | 1.776242983 | 3.4 | 0.000106 | 1 |
| hsa-miR-616_st | 0.119423732 | 1.1 | 0.566473 | 0 |
| hsa-miR-151-5p_st | 0.838212927 | 1.8 | 0.001664 | 1 |
| hsa-miR-617_st | −0.127688369 | 1.1 | 0.718503 | 0 |
| hsa-miR-128_st | 1.619243053 | 3.1 | 0.001426 | 1 |
| hsa-miR-619_st | −0.19470032 | 1.1 | 0.183164 | 0 |
| hsa-miR-620_st | −0.245720876 | 1.2 | 0.08194 | 0 |
| hsa-miR-621_st | 0.051903003 | 1.0 | 0.736618 | 0 |
| hsa-let-7b_st | 0.430340932 | 1.3 | 0.295475 | 0 |
| hsa-miR-623_st | −0.285538852 | 1.2 | 0.032673 | 1 |
| hsa-miR-624_st | −0.157975269 | 1.1 | 0.224917 | 0 |
| hsa-miR-624-star_st | 0.049249659 | 1.0 | 0.850798 | 0 |
| hsa-miR-183_st | 1.835228439 | 3.6 | 0.002511 | 1 |
| hsa-miR-30b_st | 1.133882973 | 2.2 | 4.01E−05 | 1 |
| hsa-miR-626_st | −0.192364715 | 1.1 | 0.207714 | 0 |
| hsa-miR-627_st | −0.087366785 | 1.1 | 0.618808 | 0 |
| hsa-miR-628-3p_st | −0.271739776 | 1.2 | 0.559436 | 0 |
| hsa-miR-31-star_st | 0.236736977 | 1.2 | 0.827676 | 0 |
| hsa-miR-218-1-star_st | −0.464921904 | 1.4 | 0.014686 | 1 |
| hsa-miR-629-star_st | 0.586676814 | 1.5 | 0.015252 | 1 |
| hsa-miR-630_st | −0.329334252 | 1.3 | 0.338323 | 0 |
| hsa-miR-631_st | −0.207329239 | 1.2 | 0.577404 | 0 |
| hsa-miR-632_st | −0.175176225 | 1.1 | 0.448362 | 0 |
| hsa-miR-633_st | −0.129368301 | 1.1 | 0.354536 | 0 |
| hsa-miR-634_st | −0.123472246 | 1.1 | 0.746401 | 0 |
| hsa-miR-635_st | −0.318392819 | 1.2 | 0.195233 | 0 |
| hsa-miR-203_st | 1.107326704 | 2.2 | 0.168882 | 0 |
| hsa-miR-331-3p_st | 0.908208225 | 1.9 | 0.009822 | 1 |
| hsa-miR-125b_st | 0.377703553 | 1.3 | 0.174537 | 0 |
| hsa-let-7i_st | 0.533948377 | 1.4 | 0.108245 | 0 |
| hsa-miR-640_st | −0.173838782 | 1.1 | 0.282929 | 0 |
| hsa-miR-641_st | −0.021242719 | 1.0 | 0.931745 | 0 |
| hsa-miR-642_st | −0.225540976 | 1.2 | 0.117825 | 0 |
| hsa-miR-29b_st | 1.543794907 | 2.9 | 0.000862 | 1 |
| hsa-miR-122_st | −0.746781919 | 1.7 | 0.005368 | 1 |
| hsa-miR-645_st | −0.113881606 | 1.1 | 0.391144 | 0 |
| hsa-miR-646_st | −0.004961891 | 1.0 | 0.976605 | 0 |
| hsa-miR-647_st | −0.256501841 | 1.2 | 0.072392 | 0 |
| hsa-miR-648_st | −0.324997991 | 1.3 | 0.069872 | 0 |
| hsa-miR-649_st | −0.118040565 | 1.1 | 0.651879 | 0 |
| hsa-miR-182_st | 2.261841464 | 4.8 | 0.000109 | 1 |
| hsa-miR-651_st | −0.277324586 | 1.2 | 0.064918 | 0 |
| hsa-miR-652_st | 0.115362604 | 1.1 | 0.701592 | 0 |
| hsa-miR-653_st | −0.0362333 | 1.0 | 0.803638 | 0 |
| hsa-miR-654-3p_st | 0.309176672 | 1.2 | 0.595322 | 0 |
| hsa-miR-374b_st | 1.527265203 | 2.9 | 0.000203 | 1 |
| hsa-miR-130a_st | 1.012685312 | 2.0 | 0.003332 | 1 |
| hsa-miR-656_st | −0.279584019 | 1.2 | 0.075387 | 0 |
| hsa-miR-657_st | −0.145126125 | 1.1 | 0.414567 | 0 |
| hsa-miR-658_st | −0.065261546 | 1.0 | 0.710103 | 0 |
| hsa-miR-99b_st | 0.87169929 | 1.8 | 0.015517 | 1 |
| hsa-miR-660_st | 1.25280201 | 2.4 | 0.010184 | 1 |
| hsa-miR-661_st | −0.463110155 | 1.4 | 0.01184 | 1 |
| hsa-miR-30e-star_st | 1.078393814 | 2.1 | 0.030312 | 1 |
| hsa-miR-663_st | −0.141240934 | 1.1 | 0.388094 | 0 |
| hsa-miR-663b_st | −0.331507669 | 1.3 | 0.07095 | 0 |
| hsa-miR-664_st | 0.64242781 | 1.6 | 0.067628 | 0 |
| hsa-miR-664-star_st | 0.037623658 | 1.0 | 0.905561 | 0 |
| hsa-miR-30d_st | 0.891398972 | 1.9 | 0.001523 | 1 |
| hsa-miR-668_st | 0.0355101 | 1.0 | 0.880699 | 0 |
| hsa-miR-670_st | −0.44167641 | 1.4 | 0.058068 | 0 |
| hsa-miR-671-3p_st | 0.402943707 | 1.3 | 0.072075 | 0 |
| hsa-miR-671-5p_st | −0.228509395 | 1.2 | 0.060662 | 0 |
| hsa-miR-30a-star_st | 0.9088967 | 1.9 | 0.061902 | 0 |
| hsa-miR-675-star_st | −0.087143839 | 1.1 | 0.678958 | 0 |
| hsa-miR-148b_st | 1.316705685 | 2.5 | 0.001399 | 1 |
| hsa-miR-708_st | 0.442310645 | 1.4 | 0.460724 | 0 |
| hsa-miR-708-star_st | −0.153204931 | 1.1 | 0.327227 | 0 |
| hsa-miR-221-star_st | 0.983264586 | 2.0 | 0.143558 | 0 |
| hsa-let-7e_st | 0.948058979 | 1.9 | 0.005447 | 1 |
| hsa-miR-7-1-star_st | 0.981989019 | 2.0 | 0.010849 | 1 |
| hsa-miR-27b_st | 1.261757032 | 2.4 | 3.78E−05 | 1 |
| hsa-miR-34a_st | 0.933616594 | 1.9 | 0.091903 | 0 |
| hsa-miR-744_st | −0.270649582 | 1.2 | 0.092488 | 0 |
| hsa-miR-744-star_st | 0.288186931 | 1.2 | 0.137449 | 0 |
| hsa-miR-92b_st | 0.872168985 | 1.8 | 0.120481 | 0 |
| hsa-miR-759_st | −0.089503751 | 1.1 | 0.612669 | 0 |
| hsa-miR-760_st | −0.222119663 | 1.2 | 0.264488 | 0 |
| hsa-miR-92a_st | 0.906781037 | 1.9 | 0.063877 | 0 |
| hsa-miR-335_st | 1.766976679 | 3.4 | 0.000147 | 1 |
| hsa-miR-96_st | 2.48100936 | 5.6 | 0.000106 | 1 |
| hsa-miR-95_st | 1.268105136 | 2.4 | 0.007847 | 1 |
| hsa-miR-766_st | 0.078595833 | 1.1 | 0.651704 | 0 |
| hsa-miR-767-3p_st | −0.207466952 | 1.2 | 0.091216 | 0 |
| hsa-miR-767-5p_st | 0.022662849 | 1.0 | 0.894326 | 0 |
| hsa-miR-98_st | 1.32058169 | 2.5 | 0.001374 | 1 |
| hsa-miR-769-5p_st | 0.423470618 | 1.3 | 0.17277 | 0 |
| hsa-miR-770-5p_st | 0.094198435 | 1.1 | 0.657278 | 0 |
| hsa-miR-802_st | −0.125411804 | 1.1 | 0.373819 | 0 |
| hsa-miR-34b-star_st | 1.232597395 | 2.3 | 0.018786 | 1 |
| hsa-miR-581_st | −0.468929481 | 1.4 | 0.074222 | 0 |
| hsa-miR-181d_st | 0.836844503 | 1.8 | 0.016099 | 1 |
| hsa-miR-875-5p_st | −0.162558773 | 1.1 | 0.342197 | 0 |
| hsa-miR-876-3p_st | −0.12123653 | 1.1 | 0.435903 | 0 |
| hsa-miR-876-5p_st | −0.298559327 | 1.2 | 0.124951 | 0 |
| hsa-miR-151-3p_st | 1.432754576 | 2.7 | 1.19E−05 | 1 |
| hsa-miR-877-star_st | −0.290268099 | 1.2 | 0.269744 | 0 |
| hsa-miR-885-3p_st | −0.254669598 | 1.2 | 0.248955 | 0 |
| hsa-miR-200c_st | 0.839055271 | 1.8 | 0.001917 | 1 |
| hsa-miR-429_st | 0.572430596 | 1.5 | 0.398287 | 0 |
| hsa-miR-886-5p_st | 0.174128208 | 1.1 | 0.589378 | 0 |
| hsa-miR-224_st | 1.621362833 | 3.1 | 0.007637 | 1 |
| hsa-miR-888_st | −0.086292979 | 1.1 | 0.680565 | 0 |
| hsa-miR-4321_st | −0.626340887 | 1.5 | 0.003939 | 1 |
| hsa-miR-889_st | −0.250034899 | 1.2 | 0.125379 | 0 |
| hsa-miR-181b_st | 0.978114411 | 2.0 | 0.006642 | 1 |
| hsa-miR-891a_st | 0.281716938 | 1.2 | 0.282343 | 0 |
| hsa-miR-891b_st | 0.049144674 | 1.0 | 0.793571 | 0 |
| hsa-miR-892a_st | −0.115793847 | 1.1 | 0.712346 | 0 |
| hsa-miR-892b_st | 0.037081424 | 1.0 | 0.839625 | 0 |
| hsa-miR-9_st | −0.158311541 | 1.1 | 0.338581 | 0 |
| hsa-miR-222_st | 1.115543372 | 2.2 | 0.13968 | 0 |
| hsa-miR-141_st | 1.558355536 | 2.9 | 0.000125 | 1 |
| hsa-miR-221_st | 1.258954423 | 2.4 | 0.065194 | 0 |
| hsa-miR-924_st | −0.020805508 | 1.0 | 0.842695 | 0 |
| hsa-miR-181a_st | 1.043508083 | 2.1 | 0.025651 | 1 |
| hsa-miR-181c_st | 1.190333118 | 2.3 | 0.012976 | 1 |
| hsa-miR-4324_st | 0.773648613 | 1.7 | 0.105104 | 0 |
| hsa-miR-125a-5p_st | 0.727273781 | 1.7 | 0.101279 | 0 |
| hsa-miR-92b-star_st | −0.200995148 | 1.1 | 0.115034 | 0 |
| hsa-miR-3195_st | −0.692116959 | 1.6 | 0.001413 | 1 |
| hsa-miR-3137_st | −0.704564764 | 1.6 | 0.007236 | 1 |
| hsa-miR-934_st | −0.096561033 | 1.1 | 0.66635 | 0 |
| hsa-miR-935_st | −0.134684525 | 1.1 | 0.62588 | 0 |
| hsa-miR-551b_st | 1.07431732 | 2.1 | 0.23796 | 0 |
| hsa-miR-937_st | −0.133324731 | 1.1 | 0.63771 | 0 |
| hsa-miR-938_st | −0.038256447 | 1.0 | 0.82499 | 0 |
| hsa-miR-939_st | −0.285106046 | 1.2 | 0.033987 | 1 |
| hsa-miR-7-2-star_st | −0.23839895 | 1.2 | 0.731844 | 0 |
| hsa-miR-146b-5p_st | 1.753845263 | 3.4 | 0.055515 | 0 |
| hsa-miR-941_st | −0.200227377 | 1.1 | 0.324271 | 0 |
| hsa-miR-942_st | −0.11890718 | 1.1 | 0.502054 | 0 |
| hsa-miR-200a_st | 0.078917251 | 1.1 | 0.930234 | 0 |
| hsa-miR-944_st | −0.031230587 | 1.0 | 0.878163 | 0 |
| hsa-miR-7_st | −0.991336009 | 2.0 | 0.442726 | 0 |
| hsa-miR-375_st | 0.552650418 | 1.5 | 0.127734 | 0 |
| hsa-miR-96-star_st | 0.094358029 | 1.1 | 0.644533 | 0 |
| hsa-miR-424_st | 1.978545967 | 3.9 | 0.004507 | 1 |
| hsa-miR-181a-2-star_st | 1.726391112 | 3.3 | 0.000884 | 1 |
| hsa-miR-200b_st | 0.049617782 | 1.0 | 0.955056 | 0 |
| hsa-miR-146b-3p_st | 0.855192214 | 1.8 | 0.232998 | 0 |
| hsa-miR-99b-star_st | −0.008670433 | 1.0 | 0.964416 | 0 |
| hsa-miR-9-star_st | −0.121290889 | 1.1 | 0.610079 | 0 |

| | Log2Diff | fold change | | |
|---|---|---|---|---|
| Sample Name | (FTC vs FA) | FTC vs FA | Ttest FTC vs FA | p < 0.05 |
| hsa-miR-150_st | 0.003836 | 1.0 | 0.996655 | 0 |
| hsa-let-7a-2-star_st | 0.325804 | 1.3 | 0.175696 | 0 |
| hsa-let-7a-star_st | −0.10137 | 1.1 | 0.636749 | 0 |
| hsa-miR-513b_st | −1.02885 | 2.0 | 0.192564 | 0 |
| hsa-let-7b-star_st | 0.198184 | 1.1 | 0.669089 | 0 |
| hsa-miR-593-star_st | −0.44525 | 1.4 | 0.463581 | 0 |
| hsa-let-7c-star_st | 0.332362 | 1.3 | 0.244083 | 0 |
| hsa-miR-516a-5p_st | −0.3644 | 1.3 | 0.410034 | 0 |
| hsa-miR-142-5p_st | 0.536235 | 1.5 | 0.517933 | 0 |
| hsa-miR-650_st | 0.259015 | 1.2 | 0.241864 | 0 |
| hsa-miR-513c_st | −0.8616 | 1.8 | 0.112568 | 0 |
| hsa-miR-761_st | 0.354092 | 1.3 | 0.378996 | 0 |
| hsa-let-7f-1-star_st | 0.013547 | 1.0 | 0.957907 | 0 |
| hsa-miR-513a-5p_st | −0.51318 | 1.4 | 0.33953 | 0 |
| hsa-miR-654-5p_st | −0.47094 | 1.4 | 0.2192 | 0 |
| hsa-let-7g-star_st | 0.257717 | 1.2 | 0.113322 | 0 |
| hsa-miR-3163_st | −0.22633 | 1.2 | 0.501702 | 0 |
| hsa-let-7i-star_st | 0.561389 | 1.5 | 0.17176 | 0 |
| hsa-miR-3165_st | −0.50593 | 1.4 | 0.393862 | 0 |
| hsa-miR-3194_st | −0.42498 | 1.3 | 0.205253 | 0 |
| hsa-miR-100-star_st | −0.27434 | 1.2 | 0.257596 | 0 |
| hsa-miR-4251_st | −0.50621 | 1.4 | 0.160794 | 0 |
| hsa-miR-101-star_st | 0.221492 | 1.2 | 0.207631 | 0 |
| hsa-miR-922_st | −0.12426 | 1.1 | 0.715252 | 0 |
| hsa-miR-103-2-star_st | 0.594554 | 1.5 | 0.076983 | 0 |
| hsa-miR-155_st | 0.542637 | 1.5 | 0.413611 | 0 |
| hsa-miR-105_st | −0.42862 | 1.3 | 0.063243 | 0 |
| hsa-miR-127-5p_st | −0.20376 | 1.2 | 0.659722 | 0 |
| hsa-miR-1286_st | −0.3218 | 1.2 | 0.370843 | 0 |
| hsa-miR-106a-star_st | −0.14304 | 1.1 | 0.513652 | 0 |
| hsa-miR-890_st | 0.403576 | 1.3 | 0.429766 | 0 |
| hsa-miR-3126-5p_st | −0.49179 | 1.4 | 0.280106 | 0 |
| hsa-miR-489_st | −0.48205 | 1.4 | 0.313892 | 0 |
| hsa-miR-10a_st | 0.59487 | 1.5 | 0.475798 | 0 |
| hsa-miR-10a-star_st | −0.2726 | 1.2 | 0.535923 | 0 |
| hsa-miR-10b_st | 1.175412 | 2.3 | 0.132391 | 0 |
| hsa-miR-10b-star_st | 0.378516 | 1.3 | 0.167565 | 0 |
| hsa-miR-1178_st | −0.14814 | 1.1 | 0.55587 | 0 |
| hsa-miR-1179_st | 0.134934 | 1.1 | 0.496792 | 0 |
| hsa-miR-1180_st | −0.13361 | 1.1 | 0.463428 | 0 |
| hsa-miR-1181_st | −0.2297 | 1.2 | 0.425063 | 0 |
| hsa-miR-3138_st | −0.59368 | 1.5 | 0.20186 | 0 |
| hsa-miR-220c_st | 0.069334 | 1.0 | 0.823321 | 0 |
| hsa-miR-510_st | −0.33613 | 1.3 | 0.441986 | 0 |
| hsa-miR-1185_st | −0.15794 | 1.1 | 0.450307 | 0 |
| hsa-miR-1193_st | −0.09507 | 1.1 | 0.47958 | 0 |
| hsa-miR-1197_st | −0.09755 | 1.1 | 0.544511 | 0 |
| hsa-miR-1200_st | −0.18223 | 1.1 | 0.271045 | 0 |
| hsa-miR-1201_st | 0.497869 | 1.4 | 0.192279 | 0 |
| hsa-miR-1202_st | −0.38505 | 1.3 | 0.088086 | 0 |
| hsa-miR-30c-1-star_st | −0.3181 | 1.2 | 0.426943 | 0 |
| hsa-miR-1204_st | −0.33967 | 1.3 | 0.172337 | 0 |
| hsa-miR-1205_st | −0.05437 | 1.0 | 0.773985 | 0 |
| hsa-miR-1206_st | 0.092767 | 1.1 | 0.601934 | 0 |
| hsa-miR-1207-3p_st | −0.24224 | 1.2 | 0.302448 | 0 |
| hsa-miR-1207-5p_st | −0.12644 | 1.1 | 0.30313 | 0 |
| hsa-miR-1208_st | −0.05562 | 1.0 | 0.813682 | 0 |
| hsa-miR-622_st | −0.07561 | 1.1 | 0.794946 | 0 |
| hsa-miR-1224-3p_st | 0.41131 | 1.3 | 0.093099 | 0 |
| hsa-miR-2116_st | −0.26403 | 1.2 | 0.41496 | 0 |
| hsa-miR-1225-3p_st | −0.0211 | 1.0 | 0.933814 | 0 |
| hsa-miR-1225-5p_st | −0.25403 | 1.2 | 0.084018 | 0 |
| hsa-miR-1226_st | −0.04371 | 1.0 | 0.886722 | 0 |
| hsa-miR-30b-star_st | −0.24461 | 1.2 | 0.497773 | 0 |
| hsa-miR-1227_st | 0.080551 | 1.1 | 0.748748 | 0 |
| hsa-miR-1295_st | −0.29175 | 1.2 | 0.357567 | 0 |
| hsa-miR-1228-star_st | −0.17726 | 1.1 | 0.303704 | 0 |
| hsa-miR-20b_st | 1.393321 | 2.6 | 0.007491 | 1 |
| hsa-miR-122-star_st | −0.07082 | 1.1 | 0.679438 | 0 |
| hsa-miR-1231_st | −0.14168 | 1.1 | 0.384212 | 0 |
| hsa-miR-1233_st | −0.1251 | 1.1 | 0.637901 | 0 |
| hsa-miR-524-5p_st | 0.490487 | 1.4 | 0.039963 | 1 |
| hsa-miR-1236_st | −0.02625 | 1.0 | 0.917008 | 0 |
| hsa-miR-873_st | 0.004392 | 1.0 | 0.990347 | 0 |
| hsa-miR-1238_st | −0.13155 | 1.1 | 0.610999 | 0 |
| hsa-miR-124_st | −0.03298 | 1.0 | 0.919044 | 0 |
| hsa-miR-1243_st | 0.109821 | 1.1 | 0.502253 | 0 |
| hsa-miR-1244_st | 0.15787 | 1.1 | 0.731159 | 0 |
| hsa-miR-1245_st | −0.35187 | 1.3 | 0.06721 | 0 |
| hsa-miR-3122_st | −0.43257 | 1.3 | 0.015308 | 1 |
| hsa-miR-134_st | −0.34478 | 1.3 | 0.1841 | 0 |
| hsa-miR-1248_st | −0.01262 | 1.0 | 0.982584 | 0 |
| hsa-miR-146a_st | 0.076817 | 1.1 | 0.906507 | 0 |
| hsa-miR-124-star_st | −0.02231 | 1.0 | 0.905552 | 0 |
| hsa-miR-1250_st | −0.30995 | 1.2 | 0.203582 | 0 |
| hsa-miR-764_st | −0.14147 | 1.1 | 0.68 | 0 |
| hsa-miR-1252_st | −0.09116 | 1.1 | 0.634445 | 0 |
| hsa-miR-223_st | 0.707102 | 1.6 | 0.33318 | 0 |
| hsa-miR-34c-3p_st | −0.42804 | 1.3 | 0.198129 | 0 |
| hsa-miR-1255a_st | 0.354796 | 1.3 | 0.29976 | 0 |
| hsa-miR-1255b_st | −0.04498 | 1.0 | 0.923466 | 0 |
| hsa-miR-1256_st | −0.25955 | 1.2 | 0.153655 | 0 |
| hsa-miR-1257_st | 0.269746 | 1.2 | 0.255212 | 0 |
| hsa-miR-199b-5p_st | 0.930589 | 1.9 | 0.338894 | 0 |
| hsa-miR-1259_st | −0.17603 | 1.1 | 0.349428 | 0 |
| hsa-miR-492_st | −0.28584 | 1.2 | 0.287361 | 0 |
| hsa-miR-3132_st | 0.115766 | 1.1 | 0.6706 | 0 |
| hsa-miR-516b_st | −0.21045 | 1.2 | 0.611988 | 0 |
| hsa-miR-4294_st | −0.47144 | 1.4 | 0.24005 | 0 |
| hsa-miR-125b-2-star_st | −0.02654 | 1.0 | 0.952393 | 0 |
| hsa-miR-126_st | 0.131316 | 1.1 | 0.679531 | 0 |
| hsa-miR-198_st | −0.28898 | 1.2 | 0.376912 | 0 |
| hsa-miR-3191_st | −0.21265 | 1.2 | 0.57663 | 0 |
| hsa-miR-1261_st | −0.20921 | 1.2 | 0.629733 | 0 |
| hsa-miR-1262_st | 0.125836 | 1.1 | 0.595311 | 0 |
| hsa-miR-1263_st | 0.035287 | 1.0 | 0.874418 | 0 |
| hsa-miR-1264_st | −0.12409 | 1.1 | 0.576297 | 0 |
| hsa-miR-1265_st | 0.238907 | 1.2 | 0.264928 | 0 |
| hsa-miR-2114-star_st | −0.51922 | 1.4 | 0.287403 | 0 |
| hsa-miR-1267_st | −0.12494 | 1.1 | 0.445954 | 0 |
| hsa-miR-1268_st | −0.2106 | 1.2 | 0.16381 | 0 |
| hsa-miR-1269_st | −0.00917 | 1.0 | 0.968741 | 0 |
| hsa-miR-509-5p_st | −0.35736 | 1.3 | 0.315176 | 0 |
| hsa-miR-1270_st | −0.10182 | 1.1 | 0.710382 | 0 |
| hsa-miR-1271_st | 0.351597 | 1.3 | 0.183804 | 0 |
| hsa-miR-4303_st | −0.18228 | 1.1 | 0.574812 | 0 |
| hsa-miR-1273_st | −0.27487 | 1.2 | 0.399895 | 0 |
| hsa-miR-583_st | 0.37343 | 1.3 | 0.264227 | 0 |
| hsa-miR-1273d_st | 0.060487 | 1.0 | 0.780517 | 0 |
| hsa-miR-127-3p_st | 0.905185 | 1.9 | 0.176428 | 0 |
| hsa-miR-522-star_st | −0.04203 | 1.0 | 0.850447 | 0 |
| hsa-miR-214-star_st | 0.380942 | 1.3 | 0.473353 | 0 |
| hsa-miR-184_st | −0.15633 | 1.1 | 0.679391 | 0 |
| hsa-miR-614_st | −0.14985 | 1.1 | 0.7256 | 0 |
| hsa-miR-526b_st | 0.22904 | 1.2 | 0.27066 | 0 |
| hsa-miR-1277_st | −0.11746 | 1.1 | 0.426858 | 0 |
| hsa-miR-1278_st | 0.152325 | 1.1 | 0.430256 | 0 |
| hsa-miR-1279_st | −0.29544 | 1.2 | 0.152811 | 0 |
| hsa-miR-409-3p_st | 0.262991 | 1.2 | 0.553498 | 0 |
| hsa-miR-199a-5p_st | 0.442479 | 1.4 | 0.557219 | 0 |
| hsa-miR-1281_st | −0.40291 | 1.3 | 0.142918 | 0 |
| hsa-miR-1282_st | −0.23257 | 1.2 | 0.452536 | 0 |
| hsa-miR-33b-star_st | −0.18028 | 1.1 | 0.313987 | 0 |
| hsa-miR-1284_st | 0.049141 | 1.0 | 0.858039 | 0 |
| hsa-miR-1253_st | 0.025939 | 1.0 | 0.913677 | 0 |
| hsa-miR-584_st | −0.4196 | 1.3 | 0.182109 | 0 |
| hsa-miR-1287_st | 0.311008 | 1.2 | 0.178138 | 0 |
| hsa-miR-15a_st | 1.310073 | 2.5 | 0.001717 | 1 |
| hsa-miR-1289_st | −0.04316 | 1.0 | 0.877156 | 0 |
| hsa-miR-1290_st | 0.181996 | 1.1 | 0.594639 | 0 |
| hsa-miR-2117_st | −0.38672 | 1.3 | 0.124999 | 0 |
| hsa-miR-1292_st | −0.38287 | 1.3 | 0.080214 | 0 |
| hsa-miR-1293_st | −0.17898 | 1.1 | 0.339154 | 0 |
| hsa-miR-129-3p_st | −0.22743 | 1.2 | 0.387779 | 0 |
| hsa-miR-520d-5p_st | 0.17157 | 1.1 | 0.443027 | 0 |
| hsa-miR-526a_st | 0.1606 | 1.1 | 0.513446 | 0 |
| hsa-miR-129-5p_st | −0.11485 | 1.1 | 0.548517 | 0 |
| hsa-miR-1254_st | −0.4552 | 1.4 | 0.150491 | 0 |
| hsa-miR-1297_st | −0.33044 | 1.3 | 0.095622 | 0 |
| hsa-miR-1298_st | −0.00101 | 1.0 | 0.997089 | 0 |
| hsa-miR-1299_st | −0.3758 | 1.3 | 0.46307 | 0 |
| hsa-miR-129-star_st | −0.03298 | 1.0 | 0.899993 | 0 |
| hsa-miR-1301_st | −0.03646 | 1.0 | 0.802275 | 0 |
| hsa-miR-19b_st | 1.184416 | 2.3 | 0.005216 | 1 |
| hsa-miR-1303_st | 0.157821 | 1.1 | 0.452521 | 0 |
| hsa-miR-1304_st | −0.1963 | 1.1 | 0.588294 | 0 |
| hsa-miR-1305_st | −0.18201 | 1.1 | 0.369098 | 0 |
| hsa-miR-550_st | −0.20556 | 1.2 | 0.439316 | 0 |
| hsa-miR-1307_st | −0.12244 | 1.1 | 0.531195 | 0 |
| hsa-miR-1308_st | −0.14495 | 1.1 | 0.616316 | 0 |
| hsa-miR-4291_st | 0.802479 | 1.7 | 0.047259 | 1 |
| hsa-miR-130a-star_st | 0.109584 | 1.1 | 0.61411 | 0 |
| hsa-miR-130b_st | 0.465996 | 1.4 | 0.197674 | 0 |
| hsa-miR-130b-star_st | 0.394666 | 1.3 | 0.190611 | 0 |
| hsa-miR-921_st | −0.40363 | 1.3 | 0.222751 | 0 |
| hsa-miR-520a-5p_st | 0.142514 | 1.1 | 0.484687 | 0 |
| hsa-miR-711_st | −0.13196 | 1.1 | 0.600093 | 0 |
| hsa-miR-1323_st | −0.32687 | 1.3 | 0.089014 | 0 |
| hsa-miR-1324_st | −0.22425 | 1.2 | 0.375327 | 0 |
| hsa-miR-490-3p_st | 0.023188 | 1.0 | 0.910049 | 0 |
| hsa-miR-718_st | −0.3028 | 1.2 | 0.257337 | 0 |
| hsa-miR-133b_st | 0.352275 | 1.3 | 0.714409 | 0 |
| hsa-miR-431-star_st | −0.03674 | 1.0 | 0.848118 | 0 |
| hsa-miR-552_st | −0.3833 | 1.3 | 0.272048 | 0 |
| hsa-miR-28-3p_st | 1.112476 | 2.2 | 0.039397 | 1 |
| hsa-miR-4265_st | −0.2392 | 1.2 | 0.259457 | 0 |
| hsa-miR-643_st | −0.22475 | 1.2 | 0.450376 | 0 |
| hsa-miR-136_st | −0.19257 | 1.1 | 0.242366 | 0 |
| hsa-miR-136-star_st | −0.28907 | 1.2 | 0.106537 | 0 |
| hsa-miR-137_st | −0.2567 | 1.2 | 0.098722 | 0 |
| hsa-miR-138_st | −0.67951 | 1.6 | 0.274221 | 0 |
| hsa-miR-138-1-star_st | −0.23227 | 1.2 | 0.540071 | 0 |
| hsa-miR-138-2-star_st | 0.115334 | 1.1 | 0.592604 | 0 |
| hsa-miR-139-3p_st | 0.168319 | 1.1 | 0.525832 | 0 |
| hsa-miR-139-5p_st | −0.4915 | 1.4 | 0.390058 | 0 |
| hsa-miR-140-3p_st | 0.213999 | 1.2 | 0.509873 | 0 |
| hsa-miR-140-5p_st | 0.594881 | 1.5 | 0.178993 | 0 |
| hsa-miR-381_st | 0.410303 | 1.3 | 0.14224 | 0 |
| hsa-miR-141-star_st | −0.01286 | 1.0 | 0.947046 | 0 |
| hsa-miR-17-star_st | 1.069325 | 2.1 | 0.001398 | 1 |
| hsa-miR-4300_st | 0.249647 | 1.2 | 0.324799 | 0 |
| hsa-miR-143_st | 0.099073 | 1.1 | 0.814723 | 0 |
| hsa-miR-143-star_st | 0.064001 | 1.0 | 0.886892 | 0 |
| hsa-miR-144_st | −0.03104 | 1.0 | 0.873945 | 0 |
| hsa-miR-3198_st | −0.2791 | 1.2 | 0.24046 | 0 |
| hsa-miR-145_st | −0.23029 | 1.2 | 0.626408 | 0 |
| hsa-miR-145-star_st | 0.338401 | 1.3 | 0.578648 | 0 |
| hsa-miR-1468_st | −0.05853 | 1.0 | 0.842844 | 0 |
| hsa-miR-1469_st | −0.22001 | 1.2 | 0.255943 | 0 |
| hsa-miR-1973_st | −0.98965 | 2.0 | 0.035477 | 1 |
| hsa-miR-3154_st | −0.21282 | 1.2 | 0.235722 | 0 |
| hsa-miR-148a-star_st | 0.11257 | 1.1 | 0.777449 | 0 |
| hsa-miR-92a-1-star_st | −0.09235 | 1.1 | 0.501626 | 0 |
| hsa-miR-147_st | −0.24518 | 1.2 | 0.259086 | 0 |
| hsa-miR-194-star_st | 0.051873 | 1.0 | 0.850738 | 0 |
| hsa-miR-1471_st | −0.60885 | 1.5 | 0.059959 | 0 |
| hsa-miR-147b_st | 0.003271 | 1.0 | 0.984504 | 0 |
| hsa-miR-148a_st | 0.268362 | 1.2 | 0.651317 | 0 |
| hsa-miR-4299_st | −0.63659 | 1.6 | 0.26584 | 0 |
| hsa-miR-135a-star_st | −0.50347 | 1.4 | 0.047347 | 1 |
| hsa-miR-612_st | −0.24754 | 1.2 | 0.299792 | 0 |
| hsa-miR-1321_st | −0.05212 | 1.0 | 0.817109 | 0 |
| hsa-miR-149-star_st | −0.24344 | 1.2 | 0.12765 | 0 |
| hsa-miR-604_st | −0.70652 | 1.6 | 0.115743 | 0 |
| hsa-miR-150-star_st | −0.23793 | 1.2 | 0.142551 | 0 |
| hsa-miR-125b-1-star_st | −0.28508 | 1.2 | 0.309505 | 0 |
| hsa-miR-1183_st | −0.25275 | 1.2 | 0.309972 | 0 |
| hsa-miR-152_st | 0.518116 | 1.4 | 0.184197 | 0 |
| hsa-miR-153_st | −0.29271 | 1.2 | 0.243131 | 0 |
| hsa-miR-1537_st | −0.01666 | 1.0 | 0.932074 | 0 |
| hsa-miR-1538_st | −0.40459 | 1.3 | 0.129828 | 0 |
| hsa-miR-1539_st | 0.096235 | 1.1 | 0.527217 | 0 |
| hsa-miR-154_st | −0.19224 | 1.1 | 0.628882 | 0 |
| hsa-miR-154-star_st | −0.0838 | 1.1 | 0.72676 | 0 |
| hsa-miR-432-star_st | 0.154459 | 1.1 | 0.568277 | 0 |
| hsa-miR-155-star_st | −0.04258 | 1.0 | 0.82719 | 0 |
| hsa-miR-298_st | −0.13708 | 1.1 | 0.580294 | 0 |
| hsa-miR-15a-star_st | −0.33585 | 1.3 | 0.298625 | 0 |
| hsa-miR-2110_st | −0.21991 | 1.2 | 0.525684 | 0 |
| hsa-miR-15b-star_st | −0.04254 | 1.0 | 0.843238 | 0 |
| hsa-miR-1266_st | −0.25442 | 1.2 | 0.279239 | 0 |
| hsa-miR-16-1-star_st | −0.06886 | 1.0 | 0.63305 | 0 |
| hsa-miR-16-2-star_st | −0.12532 | 1.1 | 0.695799 | 0 |
| hsa-miR-490-5p_st | −0.20496 | 1.2 | 0.434725 | 0 |
| hsa-miR-19a_st | 0.939436 | 1.9 | 0.035645 | 1 |
| hsa-miR-3192_st | −0.06433 | 1.0 | 0.771187 | 0 |
| hsa-miR-3155_st | −0.11677 | 1.1 | 0.615847 | 0 |
| hsa-miR-187-star_st | −0.38009 | 1.3 | 0.030786 | 1 |
| hsa-miR-16_st | 0.879417 | 1.8 | 0.003576 | 1 |
| hsa-miR-936_st | 0.067135 | 1.0 | 0.655372 | 0 |
| hsa-miR-181c-star_st | −0.16313 | 1.1 | 0.607546 | 0 |
| hsa-miR-379_st | 0.279999 | 1.2 | 0.496459 | 0 |
| hsa-miR-370_st | −0.54476 | 1.5 | 0.015404 | 1 |
| hsa-miR-541_st | −0.16408 | 1.1 | 0.422944 | 0 |
| hsa-miR-518c-star_st | 0.074397 | 1.1 | 0.690127 | 0 |
| hsa-miR-487b_st | 0.878813 | 1.8 | 0.039817 | 1 |
| hsa-miR-182-star_st | −0.00021 | 1.0 | 0.998904 | 0 |
| hsa-miR-3121_st | −0.4014 | 1.3 | 0.418844 | 0 |
| hsa-miR-3190-5p_st | −0.09848 | 1.1 | 0.613653 | 0 |
| hsa-miR-1203_st | −0.09492 | 1.1 | 0.661261 | 0 |
| hsa-miR-185_st | 0.288917 | 1.2 | 0.341366 | 0 |
| hsa-miR-185-star_st | −0.04197 | 1.0 | 0.874154 | 0 |
| hsa-miR-4317_st | −0.32789 | 1.3 | 0.212118 | 0 |
| hsa-miR-186-star_st | −0.26357 | 1.2 | 0.168196 | 0 |
| hsa-miR-187_st | 0.424559 | 1.3 | 0.361429 | 0 |
| hsa-miR-4257_st | −0.3684 | 1.3 | 0.099727 | 0 |
| hsa-miR-188-3p_st | 0.21871 | 1.2 | 0.429532 | 0 |
| hsa-miR-1184_st | −0.08692 | 1.1 | 0.694332 | 0 |
| hsa-miR-18a_st | 0.590025 | 1.5 | 0.063609 | 0 |
| hsa-miR-18a-star_st | −0.05358 | 1.0 | 0.842285 | 0 |
| hsa-miR-3140_st | 0.267417 | 1.2 | 0.273013 | 0 |
| hsa-miR-18b-star_st | −0.12582 | 1.1 | 0.402835 | 0 |
| hsa-miR-190_st | −0.52568 | 1.4 | 0.055844 | 0 |
| hsa-miR-601_st | −0.59045 | 1.5 | 0.098543 | 0 |
| hsa-miR-1909_st | −0.01963 | 1.0 | 0.912385 | 0 |
| hsa-miR-342-5p_st | 0.58679 | 1.5 | 0.207251 | 0 |
| hsa-miR-575_st | −0.46688 | 1.4 | 0.163002 | 0 |
| hsa-miR-885-5p_st | 0.217186 | 1.2 | 0.570236 | 0 |
| hsa-miR-300_st | 0.1799 | 1.1 | 0.447808 | 0 |
| hsa-miR-1911_st | 0.04549 | 1.0 | 0.790064 | 0 |
| hsa-miR-1911-star_st | 0.23366 | 1.2 | 0.381517 | 0 |
| hsa-miR-1912_st | −0.03857 | 1.0 | 0.840783 | 0 |
| hsa-miR-4266_st | 0.234489 | 1.2 | 0.331129 | 0 |
| hsa-miR-1914_st | 0.161852 | 1.1 | 0.501282 | 0 |
| hsa-miR-3190-3p_st | −0.2421 | 1.2 | 0.244911 | 0 |
| hsa-miR-618_st | 0.020008 | 1.0 | 0.896828 | 0 |
| hsa-miR-1276_st | −0.00159 | 1.0 | 0.995653 | 0 |
| hsa-miR-191-star_st | −0.22441 | 1.2 | 0.473402 | 0 |
| hsa-miR-1258_st | −0.12626 | 1.1 | 0.594195 | 0 |
| hsa-miR-192-star_st | −0.01681 | 1.0 | 0.928652 | 0 |
| hsa-miR-193a-3p_st | 0.318091 | 1.2 | 0.5794 | 0 |
| hsa-miR-193a-5p_st | −0.1513 | 1.1 | 0.439416 | 0 |
| hsa-miR-3157_st | −0.12115 | 1.1 | 0.585992 | 0 |
| hsa-miR-193b-star_st | −0.05019 | 1.0 | 0.765973 | 0 |
| hsa-miR-194_st | 1.054228 | 2.1 | 0.07215 | 0 |
| hsa-miR-4307_st | 0.162377 | 1.1 | 0.559416 | 0 |
| hsa-miR-195_st | 0.315144 | 1.2 | 0.482844 | 0 |
| hsa-miR-610_st | 0.102312 | 1.1 | 0.736562 | 0 |
| hsa-miR-196a_st | −0.49243 | 1.4 | 0.094788 | 0 |
| hsa-miR-196a-star_st | −0.63025 | 1.5 | 0.264187 | 0 |
| hsa-miR-196b_st | −0.45355 | 1.4 | 0.0835 | 0 |
| hsa-miR-196b-star_st | 0.130945 | 1.1 | 0.652363 | 0 |
| hsa-let-7f-2-star_st | 0.234126 | 1.2 | 0.146362 | 0 |
| hsa-miR-1972_st | −0.11039 | 1.1 | 0.673881 | 0 |
| hsa-miR-3184_st | −0.01737 | 1.0 | 0.951014 | 0 |
| hsa-miR-523-star_st | −0.00534 | 1.0 | 0.985871 | 0 |
| hsa-miR-1976_st | 0.098332 | 1.1 | 0.758766 | 0 |
| hsa-miR-625-star_st | −0.01378 | 1.0 | 0.943011 | 0 |
| hsa-miR-587_st | −0.37131 | 1.3 | 0.219819 | 0 |
| hsa-miR-199a-3p_st | 0.524178 | 1.4 | 0.438371 | 0 |
| hsa-miR-1910_st | −0.03165 | 1.0 | 0.825499 | 0 |
| hsa-miR-199b-3p_st | 0.389436 | 1.3 | 0.579092 | 0 |
| hsa-miR-639_st | −0.38982 | 1.3 | 0.247965 | 0 |
| hsa-miR-1909-star_st | 0.078349 | 1.1 | 0.740041 | 0 |
| hsa-miR-19a-star_st | −0.03188 | 1.0 | 0.903242 | 0 |
| hsa-miR-29c-star_st | 0.827405 | 1.8 | 0.022357 | 1 |
| hsa-miR-940_st | −0.22949 | 1.2 | 0.325235 | 0 |
| hsa-miR-19b-2-star_st | 0.053112 | 1.0 | 0.789727 | 0 |
| hsa-miR-4269_st | 0.156873 | 1.1 | 0.458241 | 0 |
| hsa-miR-200a-star_st | −0.12614 | 1.1 | 0.701441 | 0 |
| hsa-miR-1182_st | −0.09861 | 1.1 | 0.547573 | 0 |
| hsa-miR-518f-star_st | 0.275252 | 1.2 | 0.200814 | 0 |
| hsa-miR-92a-2-star_st | 0.015203 | 1.0 | 0.95393 | 0 |
| hsa-miR-200c-star_st | −0.18166 | 1.1 | 0.499049 | 0 |
| hsa-miR-202_st | −0.31536 | 1.2 | 0.1793 | 0 |
| hsa-miR-202-star_st | −0.21291 | 1.2 | 0.331878 | 0 |
| hsa-miR-1914-star_st | −0.33985 | 1.3 | 0.197798 | 0 |
| hsa-miR-204_st | −0.63928 | 1.6 | 0.576356 | 0 |
| hsa-miR-665_st | 0.094965 | 1.1 | 0.633859 | 0 |
| hsa-miR-2052_st | −0.29051 | 1.2 | 0.248245 | 0 |
| hsa-miR-2053_st | −0.14261 | 1.1 | 0.474388 | 0 |
| hsa-miR-2054_st | −0.33495 | 1.3 | 0.267716 | 0 |
| hsa-miR-205-star_st | 0.101211 | 1.1 | 0.712732 | 0 |
| hsa-miR-206_st | 0.469757 | 1.4 | 0.550088 | 0 |
| hsa-miR-208a_st | −0.16011 | 1.1 | 0.53918 | 0 |
| hsa-miR-208b_st | −0.19158 | 1.1 | 0.434909 | 0 |
| hsa-miR-18b_st | 0.312286 | 1.2 | 0.106268 | 0 |
| hsa-miR-20a-star_st | −0.02928 | 1.0 | 0.905583 | 0 |
| hsa-miR-3181_st | −0.39349 | 1.3 | 0.09546 | 0 |
| hsa-miR-615-5p_st | −0.1187 | 1.1 | 0.683267 | 0 |
| hsa-miR-4259_st | −0.28231 | 1.2 | 0.175944 | 0 |
| hsa-miR-210_st | −0.22271 | 1.2 | 0.152454 | 0 |
| hsa-miR-211_st | −0.38606 | 1.3 | 0.159651 | 0 |
| hsa-miR-887_st | 0.045794 | 1.0 | 0.779471 | 0 |
| hsa-miR-2113_st | 0.054839 | 1.0 | 0.847355 | 0 |
| hsa-miR-2114_st | 0.22601 | 1.2 | 0.400624 | 0 |
| hsa-miR-943_st | −0.33372 | 1.3 | 0.28924 | 0 |
| hsa-miR-2115_st | −0.1731 | 1.1 | 0.502227 | 0 |
| hsa-miR-2115-star_st | −0.56217 | 1.5 | 0.208313 | 0 |
| hsa-miR-183-star_st | −0.16789 | 1.1 | 0.464559 | 0 |
| hsa-miR-2116-star_st | −0.09377 | 1.1 | 0.74101 | 0 |
| hsa-miR-920_st | −0.08936 | 1.1 | 0.712158 | 0 |
| hsa-miR-212_st | 0.555679 | 1.5 | 0.050331 | 0 |
| hsa-miR-214_st | 0.318007 | 1.2 | 0.258146 | 0 |
| hsa-miR-1825_st | 0.013224 | 1.0 | 0.964096 | 0 |
| hsa-miR-215_st | 0.455728 | 1.4 | 0.317787 | 0 |
| hsa-miR-216a_st | −0.01247 | 1.0 | 0.946598 | 0 |
| hsa-miR-195-star_st | −0.00815 | 1.0 | 0.98036 | 0 |
| hsa-miR-217_st | 0.025238 | 1.0 | 0.836272 | 0 |
| hsa-miR-23a-star_st | −0.09992 | 1.1 | 0.603459 | 0 |
| hsa-miR-25-star_st | −0.06127 | 1.0 | 0.747359 | 0 |
| hsa-miR-218-2-star_st | −0.36215 | 1.3 | 0.054017 | 0 |
| hsa-miR-1273c_st | −0.2269 | 1.2 | 0.284232 | 0 |
| hsa-miR-219-2-3p_st | 0.008697 | 1.0 | 0.9686 | 0 |
| hsa-miR-637_st | −0.21539 | 1.2 | 0.273647 | 0 |
| hsa-miR-21-star_st | 0.351195 | 1.3 | 0.45782 | 0 |
| hsa-miR-22_st | 0.264031 | 1.2 | 0.515382 | 0 |
| hsa-miR-220a_st | −0.03039 | 1.0 | 0.864642 | 0 |
| hsa-miR-220b_st | 0.22936 | 1.2 | 0.121463 | 0 |
| hsa-miR-219-1-3p_st | 0.121044 | 1.1 | 0.588524 | 0 |
| hsa-miR-4316_st | −0.0366 | 1.0 | 0.863921 | 0 |
| hsa-miR-877_st | −0.35586 | 1.3 | 0.093057 | 0 |
| hsa-miR-636_st | −0.31417 | 1.2 | 0.226602 | 0 |
| hsa-miR-3177_st | −0.25318 | 1.2 | 0.252259 | 0 |
| hsa-miR-1249_st | 0.204442 | 1.2 | 0.544919 | 0 |
| hsa-miR-223-star_st | 0.110664 | 1.1 | 0.596729 | 0 |
| hsa-miR-1224-5p_st | −0.24767 | 1.2 | 0.047312 | 1 |
| hsa-miR-564_st | 0.003984 | 1.0 | 0.983171 | 0 |
| hsa-miR-2276_st | −0.28163 | 1.2 | 0.325487 | 0 |
| hsa-miR-2277_st | −0.11621 | 1.1 | 0.671345 | 0 |
| hsa-miR-2278_st | −0.11692 | 1.1 | 0.63711 | 0 |
| hsa-miR-22-star_st | 0.447054 | 1.4 | 0.375115 | 0 |
| hsa-miR-2355_st | 0.18533 | 1.1 | 0.285112 | 0 |
| hsa-miR-769-3p_st | 0.187209 | 1.1 | 0.491316 | 0 |
| hsa-miR-1913_st | 0.097857 | 1.1 | 0.680536 | 0 |
| hsa-miR-1237_st | −0.06883 | 1.0 | 0.782384 | 0 |
| hsa-miR-1275_st | −0.17946 | 1.1 | 0.318034 | 0 |
| hsa-miR-30a_st | 0.702289 | 1.6 | 0.033484 | 1 |
| hsa-miR-20b-star_st | −0.1173 | 1.1 | 0.711515 | 0 |
| hsa-miR-24-2-star_st | 0.657574 | 1.6 | 0.102824 | 0 |
| hsa-miR-3173_st | 0.002413 | 1.0 | 0.989499 | 0 |
| hsa-miR-3148_st | −0.01982 | 1.0 | 0.939345 | 0 |
| hsa-miR-4271_st | −0.25843 | 1.2 | 0.250517 | 0 |
| hsa-miR-659_st | −0.14004 | 1.1 | 0.388542 | 0 |
| hsa-miR-26a-2-star_st | 0.004623 | 1.0 | 0.985204 | 0 |
| hsa-miR-1228_st | 0.036272 | 1.0 | 0.899249 | 0 |
| hsa-miR-26b-star_st | 0.245685 | 1.2 | 0.436264 | 0 |
| hsa-miR-3174_st | −0.22583 | 1.2 | 0.544189 | 0 |
| hsa-miR-27a-star_st | 0.435443 | 1.4 | 0.190579 | 0 |
| hsa-miR-1285_st | −0.05276 | 1.0 | 0.774395 | 0 |
| hsa-miR-483-5p_st | −0.09419 | 1.1 | 0.534096 | 0 |
| hsa-miR-1306_st | −0.32594 | 1.3 | 0.138555 | 0 |
| hsa-miR-431_st | 0.266418 | 1.2 | 0.191264 | 0 |
| hsa-miR-765_st | 0.059869 | 1.0 | 0.658546 | 0 |
| hsa-miR-2909_st | −0.09086 | 1.1 | 0.652788 | 0 |
| hsa-miR-296-3p_st | −0.12957 | 1.1 | 0.389583 | 0 |
| hsa-miR-296-5p_st | 0.409785 | 1.3 | 0.093498 | 0 |
| hsa-miR-297_st | −0.13227 | 1.1 | 0.688193 | 0 |
| hsa-miR-323b-5p_st | −0.00966 | 1.0 | 0.973028 | 0 |
| hsa-miR-299-3p_st | −0.0584 | 1.0 | 0.855487 | 0 |
| hsa-miR-299-5p_st | 0.199926 | 1.1 | 0.71785 | 0 |
| hsa-miR-487a_st | 0.652011 | 1.6 | 0.026021 | 1 |
| hsa-miR-29a-star_st | −0.2071 | 1.2 | 0.387386 | 0 |
| hsa-miR-625_st | 0.187187 | 1.1 | 0.285366 | 0 |
| hsa-miR-611_st | 0.296266 | 1.2 | 0.314694 | 0 |
| hsa-miR-3186-3p_st | 0.592138 | 1.5 | 0.01845 | 1 |
| hsa-miR-1294_st | −0.15067 | 1.1 | 0.483346 | 0 |
| hsa-miR-3147_st | −0.22716 | 1.2 | 0.13649 | 0 |
| hsa-miR-4285_st | −0.00397 | 1.0 | 0.987958 | 0 |
| hsa-miR-125a-3p_st | −0.13057 | 1.1 | 0.470488 | 0 |
| hsa-miR-301b_st | −0.24909 | 1.2 | 0.29242 | 0 |
| hsa-miR-302a_st | −0.10563 | 1.1 | 0.635504 | 0 |
| hsa-miR-518e-star_st | 0.531879 | 1.4 | 0.017386 | 1 |
| hsa-miR-302b_st | −0.43853 | 1.4 | 0.062662 | 0 |
| hsa-miR-302b-star_st | −0.41174 | 1.3 | 0.082041 | 0 |
| hsa-miR-302c_st | 0.164246 | 1.1 | 0.453346 | 0 |
| hsa-miR-302c-star_st | −0.14466 | 1.1 | 0.537231 | 0 |
| hsa-miR-302d_st | −0.22345 | 1.2 | 0.27114 | 0 |
| hsa-miR-302d-star_st | −0.37844 | 1.3 | 0.312963 | 0 |
| hsa-miR-302e_st | −0.10597 | 1.1 | 0.659759 | 0 |
| hsa-miR-302f_st | −0.16113 | 1.1 | 0.456363 | 0 |
| hsa-miR-3065-3p_st | 0.078631 | 1.1 | 0.807272 | 0 |
| hsa-miR-3130-5p_st | −0.43954 | 1.4 | 0.129325 | 0 |
| hsa-miR-3074_st | −0.06886 | 1.0 | 0.73261 | 0 |
| hsa-miR-1226-star_st | −0.13359 | 1.1 | 0.455884 | 0 |
| hsa-miR-3180-5p_st | −0.40507 | 1.3 | 0.135352 | 0 |
| hsa-miR-1229_st | 0.489727 | 1.4 | 0.018966 | 1 |
| hsa-miR-1272_st | −0.02114 | 1.0 | 0.919707 | 0 |
| hsa-miR-30c_st | 0.349158 | 1.3 | 0.166232 | 0 |
| hsa-miR-675_st | 0.066196 | 1.0 | 0.762315 | 0 |
| hsa-miR-30c-2-star_st | 0.316159 | 1.2 | 0.337064 | 0 |
| hsa-miR-1827_st | 0.480675 | 1.4 | 0.021877 | 1 |
| hsa-miR-30d-star_st | 0.163308 | 1.1 | 0.534749 | 0 |
| hsa-miR-30e_st | 0.771675 | 1.7 | 0.066487 | 0 |
| hsa-miR-1915-star_st | 0.318779 | 1.2 | 0.258521 | 0 |
| hsa-miR-1322_st | −0.07789 | 1.1 | 0.737166 | 0 |
| hsa-miR-3115_st | 0.067627 | 1.0 | 0.701797 | 0 |
| hsa-miR-3116_st | 0.06531 | 1.0 | 0.870858 | 0 |
| hsa-miR-3117_st | −0.21651 | 1.2 | 0.381911 | 0 |
| hsa-miR-3118_st | −0.11587 | 1.1 | 0.462349 | 0 |
| hsa-miR-519b-5p_st | 0.470862 | 1.4 | 0.026035 | 1 |
| hsa-miR-3120_st | −0.04621 | 1.0 | 0.85861 | 0 |
| hsa-miR-520d-3p_st | 0.017927 | 1.0 | 0.952501 | 0 |
| hsa-miR-326_st | 0.305541 | 1.2 | 0.039498 | 1 |
| hsa-miR-3123_st | −0.22243 | 1.2 | 0.379764 | 0 |
| hsa-miR-3124_st | 0.112754 | 1.1 | 0.635706 | 0 |
| hsa-miR-3125_st | 0.386197 | 1.3 | 0.213787 | 0 |
| hsa-miR-3126-3p_st | −0.22577 | 1.2 | 0.338611 | 0 |
| hsa-miR-1296_st | 0.22741 | 1.2 | 0.280658 | 0 |
| hsa-miR-3127_st | −0.5043 | 1.4 | 0.123788 | 0 |
| hsa-miR-3128_st | −0.26163 | 1.2 | 0.204875 | 0 |
| hsa-miR-3129_st | −0.15283 | 1.1 | 0.37761 | 0 |
| hsa-miR-3130-3p_st | −0.08429 | 1.1 | 0.693096 | 0 |
| hsa-miR-105-star_st | −0.21856 | 1.2 | 0.232531 | 0 |
| hsa-miR-3131_st | 0.03666 | 1.0 | 0.821258 | 0 |
| hsa-miR-4255_st | −0.00708 | 1.0 | 0.966983 | 0 |
| hsa-miR-3133_st | −0.70949 | 1.6 | 0.264718 | 0 |
| hsa-miR-3134_st | −0.15734 | 1.1 | 0.470192 | 0 |
| hsa-miR-3135_st | 0.133846 | 1.1 | 0.570501 | 0 |
| hsa-miR-3136_st | −0.38367 | 1.3 | 0.079466 | 0 |
| hsa-miR-19b-1-star_st | 0.299396 | 1.2 | 0.040925 | 1 |
| hsa-miR-1247_st | 0.1167 | 1.1 | 0.692077 | 0 |
| hsa-miR-3139_st | 0.220996 | 1.2 | 0.118611 | 0 |
| hsa-miR-596_st | 0.12537 | 1.1 | 0.610588 | 0 |
| hsa-miR-3141_st | −0.09855 | 1.1 | 0.543104 | 0 |
| hsa-miR-3142_st | −0.12099 | 1.1 | 0.458119 | 0 |
| hsa-miR-1302_st | −0.24041 | 1.2 | 0.034153 | 1 |
| hsa-miR-3144-3p_st | 0.099056 | 1.1 | 0.565169 | 0 |
| hsa-miR-3144-5p_st | 0.100303 | 1.1 | 0.641545 | 0 |
| hsa-miR-3145_st | −0.37099 | 1.3 | 0.13191 | 0 |
| hsa-miR-3146_st | −0.23063 | 1.2 | 0.375543 | 0 |
| hsa-miR-346_st | −0.44308 | 1.4 | 0.024098 | 1 |
| hsa-miR-4256_st | −0.26696 | 1.2 | 0.237085 | 0 |
| hsa-miR-3149_st | 0.039324 | 1.0 | 0.878595 | 0 |
| hsa-miR-3150_st | −0.06843 | 1.0 | 0.737875 | 0 |
| hsa-miR-3151_st | −0.02838 | 1.0 | 0.869404 | 0 |
| hsa-miR-1470_st | −0.00233 | 1.0 | 0.989822 | 0 |
| hsa-miR-3153_st | −0.1227 | 1.1 | 0.601306 | 0 |
| hsa-miR-514_st | −0.19776 | 1.1 | 0.137694 | 0 |
| hsa-miR-3175_st | −0.00683 | 1.0 | 0.956104 | 0 |
| hsa-miR-3156_st | 0.106037 | 1.1 | 0.549654 | 0 |
| hsa-miR-504_st | 0.190037 | 1.1 | 0.506183 | 0 |
| hsa-miR-3158_st | −0.02957 | 1.0 | 0.932028 | 0 |
| hsa-miR-3159_st | 0.204391 | 1.2 | 0.373196 | 0 |
| hsa-miR-3160_st | −0.1329 | 1.1 | 0.575784 | 0 |
| hsa-miR-3161_st | 0.127278 | 1.1 | 0.616169 | 0 |
| hsa-miR-3162_st | −0.24025 | 1.2 | 0.17853 | 0 |
| hsa-miR-1234_st | −0.6025 | 1.5 | 0.022837 | 1 |
| hsa-miR-3164_st | −0.21246 | 1.2 | 0.520373 | 0 |
| hsa-miR-516b-star_st | 0.045903 | 1.0 | 0.834281 | 0 |
| hsa-miR-3166_st | −0.02793 | 1.0 | 0.875235 | 0 |
| hsa-miR-4327_st | −0.30244 | 1.2 | 0.037494 | 1 |
| hsa-miR-3168_st | 0.098448 | 1.1 | 0.66215 | 0 |
| hsa-miR-3169_st | −0.17784 | 1.1 | 0.459052 | 0 |
| hsa-miR-3170_st | 0.148266 | 1.1 | 0.619156 | 0 |
| hsa-miR-3171_st | −0.25352 | 1.2 | 0.108188 | 0 |
| hsa-miR-3172_st | −0.12244 | 1.1 | 0.681242 | 0 |
| hsa-miR-374b-star_st | 0.19868 | 1.1 | 0.35442 | 0 |
| hsa-miR-34c-5p_st | −0.04682 | 1.0 | 0.736121 | 0 |
| hsa-miR-758_st | −0.10962 | 1.1 | 0.806436 | 0 |
| hsa-miR-3176_st | 0.083525 | 1.1 | 0.661756 | 0 |
| hsa-miR-606_st | −0.00164 | 1.0 | 0.992719 | 0 |
| hsa-miR-3178_st | −0.2091 | 1.2 | 0.240435 | 0 |
| hsa-miR-3179_st | −0.12311 | 1.1 | 0.642513 | 0 |
| hsa-miR-3180-3p_st | −0.0686 | 1.0 | 0.738367 | 0 |
| hsa-miR-597_st | 0.083757 | 1.1 | 0.605695 | 0 |
| hsa-miR-148b-star_st | 0.270953 | 1.2 | 0.191722 | 0 |
| hsa-miR-3182_st | −0.04667 | 1.0 | 0.859014 | 0 |
| hsa-miR-3183_st | −0.30176 | 1.2 | 0.255994 | 0 |
| hsa-miR-144-star_st | −0.04646 | 1.0 | 0.828943 | 0 |
| hsa-miR-3185_st | −0.20421 | 1.2 | 0.273575 | 0 |
| hsa-miR-4270_st | −0.32035 | 1.2 | 0.029384 | 1 |
| hsa-miR-3186-5p_st | −0.15868 | 1.1 | 0.43347 | 0 |
| hsa-miR-3187_st | −0.22844 | 1.2 | 0.210621 | 0 |
| hsa-miR-3188_st | −0.26753 | 1.2 | 0.098285 | 0 |
| hsa-miR-3189_st | 0.064718 | 1.0 | 0.778392 | 0 |
| hsa-miR-548b-3p_st | −0.17401 | 1.1 | 0.55048 | 0 |
| hsa-miR-99a-star_st | −0.16142 | 1.1 | 0.744987 | 0 |
| hsa-miR-376a-star_st | −0.45596 | 1.4 | 0.049213 | 1 |
| hsa-miR-216b_st | 0.225416 | 1.2 | 0.168545 | 0 |
| hsa-miR-3193_st | −0.35662 | 1.3 | 0.121107 | 0 |
| hsa-miR-548v_st | −0.26297 | 1.2 | 0.141894 | 0 |
| hsa-miR-26a-1-star_st | −0.35219 | 1.3 | 0.010771 | 1 |
| hsa-miR-1291_st | −0.44601 | 1.4 | 0.232503 | 0 |
| hsa-miR-3197_st | −0.41006 | 1.3 | 0.084926 | 0 |
| hsa-miR-629_st | 0.531976 | 1.4 | 0.049133 | 1 |
| hsa-miR-3199_st | −0.02385 | 1.0 | 0.942048 | 0 |
| hsa-miR-190b_st | −0.10866 | 1.1 | 0.50915 | 0 |
| hsa-miR-32_st | −0.09266 | 1.1 | 0.683079 | 0 |
| hsa-miR-3200_st | 0.25299 | 1.2 | 0.468814 | 0 |
| hsa-miR-3201_st | −0.12097 | 1.1 | 0.494382 | 0 |
| hsa-miR-3202_st | 0.225244 | 1.2 | 0.479236 | 0 |
| hsa-miR-320a_st | 0.201717 | 1.2 | 0.243897 | 0 |
| hsa-miR-4289_st | 0.47655 | 1.4 | 0.170494 | 0 |
| hsa-miR-655_st | 0.061635 | 1.0 | 0.778477 | 0 |
| hsa-miR-320b_st | 0.340159 | 1.3 | 0.173287 | 0 |
| hsa-miR-219-5p_st | −0.13209 | 1.1 | 0.653346 | 0 |
| hsa-miR-323-3p_st | −0.15399 | 1.1 | 0.48953 | 0 |
| hsa-miR-323-5p_st | −0.23974 | 1.2 | 0.420136 | 0 |
| hsa-miR-323b-3p_st | −0.01209 | 1.0 | 0.942858 | 0 |
| hsa-miR-103-as_st | 0.098202 | 1.1 | 0.518448 | 0 |
| hsa-miR-424-star_st | −0.16483 | 1.1 | 0.479445 | 0 |
| hsa-miR-33a-star_st | 0.426555 | 1.3 | 0.240727 | 0 |
| hsa-miR-325_st | −0.21088 | 1.2 | 0.470211 | 0 |
| hsa-miR-590-3p_st | −0.38457 | 1.3 | 0.043842 | 1 |
| hsa-miR-328_st | 0.434852 | 1.4 | 0.430281 | 0 |
| hsa-miR-329_st | 0.587996 | 1.5 | 0.174679 | 0 |
| hsa-miR-32-star_st | −0.08993 | 1.1 | 0.667123 | 0 |
| hsa-miR-330-3p_st | 0.39696 | 1.3 | 0.207683 | 0 |
| hsa-miR-330-5p_st | −0.13362 | 1.1 | 0.603698 | 0 |
| hsa-miR-146a-star_st | −0.05897 | 1.0 | 0.771533 | 0 |
| hsa-miR-331-5p_st | 0.166489 | 1.1 | 0.51487 | 0 |
| hsa-miR-3167_st | −0.38632 | 1.3 | 0.028809 | 1 |
| hsa-miR-335-star_st | −0.3671 | 1.3 | 0.271161 | 0 |
| hsa-miR-337-3p_st | −0.22181 | 1.2 | 0.457897 | 0 |
| hsa-miR-337-5p_st | 0.334032 | 1.3 | 0.050059 | 0 |
| hsa-miR-421_st | 0.940633 | 1.9 | 0.00781 | 1 |
| hsa-miR-338-5p_st | 0.391614 | 1.3 | 0.180337 | 0 |
| hsa-miR-339-3p_st | 0.008323 | 1.0 | 0.96922 | 0 |
| hsa-miR-548a-5p_st | 0.104566 | 1.1 | 0.502446 | 0 |
| hsa-miR-33a_st | −0.29315 | 1.2 | 0.11412 | 0 |
| hsa-miR-590-5p_st | 0.331661 | 1.3 | 0.147544 | 0 |
| hsa-miR-33b_st | −0.07061 | 1.1 | 0.620926 | 0 |
| hsa-miR-616-star_st | −0.02601 | 1.0 | 0.926597 | 0 |
| hsa-miR-569_st | −0.39691 | 1.3 | 0.013607 | 1 |
| hsa-miR-340-star_st | −0.07495 | 1.1 | 0.757352 | 0 |
| hsa-miR-342-3p_st | 0.858351 | 1.8 | 0.121309 | 0 |
| hsa-miR-135b-star_st | −0.80802 | 1.8 | 0.017496 | 1 |
| hsa-miR-345_st | 0.102672 | 1.1 | 0.877297 | 0 |
| hsa-miR-1288_st | −0.40285 | 1.3 | 0.022154 | 1 |
| hsa-miR-135a_st | −0.55826 | 1.5 | 0.012806 | 1 |
| hsa-miR-34a-star_st | 0.213571 | 1.2 | 0.698607 | 0 |
| hsa-miR-34b_st | 0.309455 | 1.2 | 0.366191 | 0 |
| hsa-miR-4288_st | −0.10001 | 1.1 | 0.685432 | 0 |
| hsa-miR-222-star_st | 0.496349 | 1.4 | 0.3587 | 0 |
| hsa-miR-933_st | 0.688178 | 1.6 | 0.046823 | 1 |
| hsa-miR-361-3p_st | 0.579094 | 1.5 | 0.173452 | 0 |
| hsa-miR-224-star_st | 0.438045 | 1.4 | 0.224581 | 0 |
| hsa-miR-362-3p_st | 0.546383 | 1.5 | 0.19979 | 0 |
| hsa-miR-362-5p_st | 0.714619 | 1.6 | 0.100695 | 0 |
| hsa-miR-363_st | 1.016977 | 2.0 | 0.141305 | 0 |
| hsa-miR-363-star_st | 0.035825 | 1.0 | 0.8785 | 0 |
| hsa-miR-1979_st | −0.04916 | 1.0 | 0.816015 | 0 |
| hsa-miR-365-star_st | −0.12101 | 1.1 | 0.471102 | 0 |
| hsa-miR-548x_st | −0.41819 | 1.3 | 0.0259 | 1 |
| hsa-miR-367-star_st | 0.035199 | 1.0 | 0.865461 | 0 |
| hsa-miR-369-3p_st | −0.20494 | 1.2 | 0.37928 | 0 |
| hsa-miR-369-5p_st | 0.392598 | 1.3 | 0.311119 | 0 |
| hsa-miR-548l_st | −0.41882 | 1.3 | 0.027411 | 1 |
| hsa-miR-371-3p_st | −0.18771 | 1.1 | 0.241609 | 0 |
| hsa-miR-371-5p_st | −0.2094 | 1.2 | 0.144608 | 0 |
| hsa-miR-372_st | −0.02567 | 1.0 | 0.905443 | 0 |
| hsa-miR-373_st | 0.146916 | 1.1 | 0.570795 | 0 |
| hsa-miR-373-star_st | −0.04614 | 1.0 | 0.80902 | 0 |
| hsa-miR-374a_st | 0.530081 | 1.4 | 0.405606 | 0 |
| hsa-miR-374a-star_st | 0.340761 | 1.3 | 0.08856 | 0 |
| hsa-miR-500_st | 0.121881 | 1.1 | 0.771509 | 0 |
| hsa-miR-320c_st | 0.490756 | 1.4 | 0.082508 | 0 |
| hsa-miR-875-3p_st | −0.41951 | 1.3 | 0.002608 | 1 |
| hsa-miR-376a_st | 0.390638 | 1.3 | 0.377488 | 0 |
| hsa-miR-29b-1-star_st | 1.182829 | 2.3 | 0.003987 | 1 |
| hsa-miR-376b_st | 0.224039 | 1.2 | 0.518173 | 0 |
| hsa-miR-376c_st | 0.482687 | 1.4 | 0.447971 | 0 |
| hsa-miR-377_st | −0.20184 | 1.2 | 0.572739 | 0 |
| hsa-miR-377-star_st | −0.05553 | 1.0 | 0.862219 | 0 |
| hsa-miR-378_st | 0.034874 | 1.0 | 0.954931 | 0 |
| hsa-miR-378b_st | 0.003396 | 1.0 | 0.984449 | 0 |
| hsa-miR-378c_st | 0.45578 | 1.4 | 0.291792 | 0 |
| hsa-miR-378-star_st | 0.764584 | 1.7 | 0.077335 | 0 |
| hsa-miR-662_st | 0.253522 | 1.2 | 0.340426 | 0 |
| hsa-miR-379-star_st | −0.04509 | 1.0 | 0.819968 | 0 |
| hsa-miR-380_st | −0.16262 | 1.1 | 0.390033 | 0 |
| hsa-miR-380-star_st | 0.130985 | 1.1 | 0.385396 | 0 |
| hsa-miR-1274b_st | 0.188432 | 1.1 | 0.454347 | 0 |
| hsa-miR-382_st | −0.05523 | 1.0 | 0.912865 | 0 |
| hsa-miR-383_st | 0.003645 | 1.0 | 0.986134 | 0 |
| hsa-miR-384_st | 0.017468 | 1.0 | 0.93792 | 0 |
| hsa-miR-126-star_st | −0.29811 | 1.2 | 0.189104 | 0 |
| hsa-miR-409-5p_st | 0.425177 | 1.3 | 0.122636 | 0 |
| hsa-miR-410_st | 0.144684 | 1.1 | 0.462728 | 0 |
| hsa-miR-411_st | 0.149425 | 1.1 | 0.549403 | 0 |
| hsa-miR-411-star_st | 0.064291 | 1.0 | 0.822688 | 0 |
| hsa-miR-412_st | −0.19843 | 1.1 | 0.428019 | 0 |
| hsa-miR-3143_st | −0.43483 | 1.4 | 0.048262 | 1 |
| hsa-miR-422a_st | 0.350583 | 1.3 | 0.393672 | 0 |
| hsa-miR-628-5p_st | −0.12278 | 1.1 | 0.578758 | 0 |
| hsa-miR-423-5p_st | −0.08309 | 1.1 | 0.586259 | 0 |
| hsa-miR-27b-star_st | 1.095141 | 2.1 | 0.002161 | 1 |
| hsa-miR-499-5p_st | 0.298145 | 1.2 | 0.33479 | 0 |
| hsa-miR-425_st | 0.708003 | 1.6 | 0.159849 | 0 |
| hsa-miR-193b_st | 1.113699 | 2.2 | 0.084179 | 0 |
| hsa-miR-4252_st | −0.14389 | 1.1 | 0.388612 | 0 |
| hsa-miR-4253_st | 0.058779 | 1.0 | 0.729028 | 0 |
| hsa-miR-4254_st | 0.355498 | 1.3 | 0.26767 | 0 |
| hsa-miR-324-3p_st | 0.034823 | 1.0 | 0.902073 | 0 |
| hsa-miR-4329_st | 0.223581 | 1.2 | 0.286302 | 0 |
| hsa-miR-1251_st | 0.466155 | 1.4 | 0.328518 | 0 |
| hsa-miR-4258_st | −0.31435 | 1.2 | 0.357111 | 0 |
| hsa-miR-1274a_st | 0.519908 | 1.4 | 0.26933 | 0 |
| hsa-miR-425-star_st | 0.164478 | 1.1 | 0.422277 | 0 |
| hsa-miR-4260_st | −0.22976 | 1.2 | 0.244278 | 0 |
| hsa-miR-4261_st | −0.25639 | 1.2 | 0.213052 | 0 |
| hsa-miR-4262_st | −0.35016 | 1.3 | 0.074157 | 0 |
| hsa-miR-4263_st | −0.17105 | 1.1 | 0.376312 | 0 |
| hsa-miR-4264_st | −0.29874 | 1.2 | 0.14594 | 0 |
| hsa-miR-1280_st | −0.07122 | 1.1 | 0.75145 | 0 |
| hsa-miR-132-star_st | 0.644864 | 1.6 | 0.317217 | 0 |
| hsa-miR-4267_st | −0.10687 | 1.1 | 0.69977 | 0 |
| hsa-miR-4268_st | −0.39414 | 1.3 | 0.08207 | 0 |
| hsa-miR-133a_st | 0.251796 | 1.2 | 0.789324 | 0 |
| hsa-miR-888-star_st | −0.44154 | 1.4 | 0.01369 | 1 |
| hsa-miR-1260b_st | −0.04105 | 1.0 | 0.848776 | 0 |
| hsa-miR-4272_st | −0.12439 | 1.1 | 0.495421 | 0 |
| hsa-miR-4273_st | −0.3116 | 1.2 | 0.118686 | 0 |
| hsa-miR-4274_st | −0.10821 | 1.1 | 0.687956 | 0 |
| hsa-miR-4275_st | 0.121695 | 1.1 | 0.575715 | 0 |
| hsa-miR-4276_st | 0.187941 | 1.1 | 0.367846 | 0 |
| hsa-miR-4277_st | −0.12748 | 1.1 | 0.475914 | 0 |
| hsa-miR-4278_st | −0.14787 | 1.1 | 0.646857 | 0 |
| hsa-miR-4279_st | 0.18407 | 1.1 | 0.495189 | 0 |
| hsa-miR-4280_st | −0.03567 | 1.0 | 0.889687 | 0 |
| hsa-miR-4281_st | −0.26993 | 1.2 | 0.106036 | 0 |
| hsa-miR-4282_st | −0.15489 | 1.1 | 0.288005 | 0 |
| hsa-miR-4283_st | −0.09179 | 1.1 | 0.686956 | 0 |
| hsa-miR-4284_st | −0.13582 | 1.1 | 0.632133 | 0 |
| hsa-miR-23b-star_st | −0.05391 | 1.0 | 0.852601 | 0 |
| hsa-miR-574-3p_st | 0.272672 | 1.2 | 0.452703 | 0 |
| hsa-miR-4287_st | 0.102424 | 1.1 | 0.636862 | 0 |
| hsa-miR-320e_st | 0.696881 | 1.6 | 0.031988 | 1 |
| hsa-miR-339-5p_st | 0.511359 | 1.4 | 0.400736 | 0 |
| hsa-miR-505_st | 0.469048 | 1.4 | 0.362337 | 0 |
| hsa-miR-4290_st | 0.268438 | 1.2 | 0.295303 | 0 |
| hsa-miR-200b-star_st | −0.18939 | 1.1 | 0.523368 | 0 |
| hsa-miR-4292_st | −0.19391 | 1.1 | 0.441846 | 0 |
| hsa-miR-4293_st | −0.04096 | 1.0 | 0.852496 | 0 |
| hsa-miR-1246_st | 0.240132 | 1.2 | 0.36473 | 0 |
| hsa-miR-4295_st | −0.01027 | 1.0 | 0.961946 | 0 |
| hsa-miR-4296_st | −0.07066 | 1.1 | 0.761157 | 0 |
| hsa-miR-4297_st | −0.29408 | 1.2 | 0.401445 | 0 |
| hsa-miR-4298_st | −0.11707 | 1.1 | 0.478885 | 0 |
| hsa-miR-218_st | 1.314195 | 2.5 | 0.020966 | 1 |
| hsa-miR-4286_st | 0.199402 | 1.1 | 0.470866 | 0 |
| hsa-miR-4301_st | −0.24181 | 1.2 | 0.371978 | 0 |
| hsa-miR-4302_st | −0.24502 | 1.2 | 0.3078 | 0 |
| hsa-miR-365_st | 0.148394 | 1.1 | 0.78099 | 0 |
| hsa-miR-4304_st | 0.173211 | 1.1 | 0.595586 | 0 |
| hsa-miR-4305_st | −0.19193 | 1.1 | 0.519671 | 0 |
| hsa-miR-4306_st | 0.118985 | 1.1 | 0.62133 | 0 |
| hsa-miR-26a_st | 0.387071 | 1.3 | 0.152162 | 0 |
| hsa-miR-4308_st | 0.254862 | 1.2 | 0.350885 | 0 |
| hsa-miR-4309_st | −0.05858 | 1.0 | 0.679624 | 0 |
| hsa-miR-186_st | 0.883374 | 1.8 | 0.151643 | 0 |
| hsa-miR-4310_st | −0.37783 | 1.3 | 0.372101 | 0 |
| hsa-miR-4311_st | 0.185968 | 1.1 | 0.482475 | 0 |
| hsa-miR-4312_st | −0.23517 | 1.2 | 0.482239 | 0 |
| hsa-miR-4313_st | −0.18373 | 1.1 | 0.507883 | 0 |
| hsa-miR-4314_st | −0.236 | 1.2 | 0.559123 | 0 |
| hsa-miR-4315_st | −0.10869 | 1.1 | 0.659939 | 0 |
| hsa-miR-149_st | 0.797217 | 1.7 | 0.106709 | 0 |
| hsa-miR-106b-star_st | 0.683908 | 1.6 | 0.068681 | 0 |
| hsa-miR-4318_st | −0.2426 | 1.2 | 0.094989 | 0 |
| hsa-miR-4319_st | 0.07725 | 1.1 | 0.843461 | 0 |
| hsa-miR-29b-2-star_st | 0.794779 | 1.7 | 0.003481 | 1 |
| hsa-miR-432_st | −0.14829 | 1.1 | 0.630156 | 0 |
| hsa-miR-4320_st | 0.117533 | 1.1 | 0.476177 | 0 |
| hsa-miR-522_st | −0.44901 | 1.4 | 0.011901 | 1 |
| hsa-miR-4322_st | −0.21066 | 1.2 | 0.184247 | 0 |
| hsa-miR-4323_st | 0.237976 | 1.2 | 0.27361 | 0 |
| hsa-miR-93-star_st | 0.652552 | 1.6 | 0.021115 | 1 |
| hsa-miR-4325_st | 0.08902 | 1.1 | 0.633461 | 0 |
| hsa-miR-4326_st | −0.13236 | 1.1 | 0.593053 | 0 |
| hsa-miR-874_st | −0.4532 | 1.4 | 0.044163 | 1 |
| hsa-miR-4328_st | −0.01423 | 1.0 | 0.938238 | 0 |
| hsa-let-7e-star_st | 0.52421 | 1.4 | 0.09974 | 0 |
| hsa-miR-720_st | −0.17549 | 1.1 | 0.398842 | 0 |
| hsa-miR-433_st | 0.153648 | 1.1 | 0.486532 | 0 |
| hsa-miR-4330_st | −0.60192 | 1.5 | 0.148637 | 0 |
| hsa-miR-448_st | −0.19046 | 1.1 | 0.243633 | 0 |
| hsa-miR-449a_st | 0.434295 | 1.4 | 0.138637 | 0 |
| hsa-miR-449b_st | −0.07026 | 1.0 | 0.76864 | 0 |
| hsa-miR-1975_st | −0.13992 | 1.1 | 0.501689 | 0 |
| hsa-miR-449c_st | −0.17983 | 1.1 | 0.583641 | 0 |
| hsa-miR-449c-star_st | −0.36638 | 1.3 | 0.143785 | 0 |
| hsa-miR-1_st | 0.596177 | 1.5 | 0.487841 | 0 |
| hsa-miR-450b-3p_st | −0.33638 | 1.3 | 0.168266 | 0 |
| hsa-miR-450b-5p_st | −0.31067 | 1.2 | 0.187935 | 0 |
| hsa-miR-451_st | 0.285634 | 1.2 | 0.627182 | 0 |
| hsa-miR-452_st | 0.451704 | 1.4 | 0.119076 | 0 |
| hsa-miR-452-star_st | −0.20887 | 1.2 | 0.183201 | 0 |
| hsa-miR-548b-5p_st | −0.48084 | 1.4 | 0.027293 | 1 |
| hsa-miR-454-star_st | 0.071639 | 1.1 | 0.772005 | 0 |
| hsa-miR-455-3p_st | 0.818152 | 1.8 | 0.129233 | 0 |
| hsa-miR-455-5p_st | 0.804104 | 1.7 | 0.124627 | 0 |
| hsa-miR-466_st | −0.04361 | 1.0 | 0.902311 | 0 |
| hsa-miR-483-3p_st | 0.047001 | 1.0 | 0.866173 | 0 |
| hsa-miR-17_st | 0.944249 | 1.9 | 0.034817 | 1 |
| hsa-miR-320d_st | 0.617733 | 1.5 | 0.099858 | 0 |
| hsa-miR-485-3p_st | 0.526894 | 1.4 | 0.147191 | 0 |
| hsa-miR-485-5p_st | 0.027605 | 1.0 | 0.900616 | 0 |
| hsa-miR-486-3p_st | 0.235261 | 1.2 | 0.425971 | 0 |
| hsa-miR-486-5p_st | −0.0171 | 1.0 | 0.979164 | 0 |
| hsa-miR-644_st | −0.48367 | 1.4 | 0.025974 | 1 |
| hsa-miR-3152_st | −0.49086 | 1.4 | 0.045215 | 1 |
| hsa-miR-488_st | −0.13166 | 1.1 | 0.510634 | 0 |
| hsa-miR-488-star_st | 0.019082 | 1.0 | 0.917241 | 0 |
| hsa-miR-450a_st | 0.750878 | 1.7 | 0.114019 | 0 |
| hsa-miR-20a_st | 1.467805 | 2.8 | 0.016524 | 1 |
| hsa-miR-484_st | 0.727815 | 1.7 | 0.105457 | 0 |
| hsa-miR-491-3p_st | −0.16755 | 1.1 | 0.468417 | 0 |
| hsa-miR-491-5p_st | −0.1459 | 1.1 | 0.597685 | 0 |
| hsa-miR-197_st | 0.203223 | 1.2 | 0.680364 | 0 |
| hsa-miR-493_st | 0.298667 | 1.2 | 0.236587 | 0 |
| hsa-miR-493-star_st | 0.478876 | 1.4 | 0.319548 | 0 |
| hsa-miR-494_st | −0.7216 | 1.6 | 0.05317 | 0 |
| hsa-miR-495_st | 0.719482 | 1.6 | 0.132115 | 0 |
| hsa-miR-496_st | −0.07983 | 1.1 | 0.622371 | 0 |
| hsa-miR-497_st | 0.159157 | 1.1 | 0.780012 | 0 |
| hsa-miR-497-star_st | −0.14108 | 1.1 | 0.286301 | 0 |
| hsa-miR-498_st | −0.07865 | 1.1 | 0.703613 | 0 |
| hsa-miR-499-3p_st | −0.0638 | 1.0 | 0.649944 | 0 |
| hsa-miR-24-1-star_st | 0.347361 | 1.3 | 0.384935 | 0 |
| hsa-miR-340_st | 1.481217 | 2.8 | 0.018919 | 1 |
| hsa-miR-500b_st | 0.387867 | 1.3 | 0.107183 | 0 |
| hsa-miR-500-star_st | 0.009939 | 1.0 | 0.954161 | 0 |
| hsa-miR-501-3p_st | −0.16712 | 1.1 | 0.298834 | 0 |
| hsa-miR-501-5p_st | −0.08768 | 1.1 | 0.756055 | 0 |
| hsa-miR-502-3p_st | 0.081153 | 1.1 | 0.64498 | 0 |
| hsa-miR-502-5p_st | −0.0222 | 1.0 | 0.916025 | 0 |
| hsa-miR-503_st | 0.15054 | 1.1 | 0.592808 | 0 |
| hsa-miR-192_st | 1.181281 | 2.3 | 0.073623 | 0 |
| hsa-let-7g_st | 0.50496 | 1.4 | 0.057548 | 0 |
| hsa-miR-505-star_st | 0.151465 | 1.1 | 0.488093 | 0 |
| hsa-miR-506_st | −0.27763 | 1.2 | 0.185458 | 0 |
| hsa-miR-507_st | 0.01962 | 1.0 | 0.935377 | 0 |
| hsa-miR-508-3p_st | −0.33804 | 1.3 | 0.130665 | 0 |
| hsa-miR-508-5p_st | 0.088618 | 1.1 | 0.668303 | 0 |
| hsa-miR-509-3-5p_st | −0.15098 | 1.1 | 0.721564 | 0 |
| hsa-miR-509-3p_st | −0.20814 | 1.2 | 0.424985 | 0 |
| hsa-miR-106a_st | 1.047486 | 2.1 | 0.025948 | 1 |
| hsa-miR-106b_st | 1.050508 | 2.1 | 0.022161 | 1 |
| hsa-miR-511_st | −0.0345 | 1.0 | 0.858261 | 0 |
| hsa-miR-512-3p_st | −0.2403 | 1.2 | 0.206756 | 0 |
| hsa-miR-512-5p_st | 0.26185 | 1.2 | 0.224538 | 0 |
| hsa-miR-513a-3p_st | −0.1855 | 1.1 | 0.403998 | 0 |
| hsa-miR-23a_st | 0.384983 | 1.3 | 0.221879 | 0 |
| hsa-miR-25_st | 0.917307 | 1.9 | 0.039209 | 1 |
| hsa-miR-598_st | 0.816706 | 1.8 | 0.080616 | 0 |
| hsa-miR-1260_st | 0.259003 | 1.2 | 0.371633 | 0 |
| hsa-miR-514b-3p_st | 0.072381 | 1.1 | 0.78519 | 0 |
| hsa-miR-514b-5p_st | 0.037278 | 1.0 | 0.825672 | 0 |
| hsa-miR-515-3p_st | −0.49193 | 1.4 | 0.041601 | 1 |
| hsa-miR-515-5p_st | −0.13187 | 1.1 | 0.448548 | 0 |
| hsa-miR-516a-3p_st | −0.2482 | 1.2 | 0.249938 | 0 |
| hsa-miR-27a_st | 0.687725 | 1.6 | 0.069102 | 0 |
| hsa-miR-100_st | 0.445035 | 1.4 | 0.331952 | 0 |
| hsa-miR-101_st | 1.170239 | 2.3 | 0.064749 | 0 |
| hsa-miR-517a_st | 0.258194 | 1.2 | 0.338358 | 0 |
| hsa-miR-517b_st | 0.00792 | 1.0 | 0.969539 | 0 |
| hsa-miR-517c_st | −0.24007 | 1.2 | 0.23638 | 0 |
| hsa-miR-517-star_st | 0.093904 | 1.1 | 0.688365 | 0 |
| hsa-miR-518a-3p_st | −0.17195 | 1.1 | 0.416557 | 0 |
| hsa-miR-518a-5p_st | −0.08273 | 1.1 | 0.77997 | 0 |
| hsa-miR-518b_st | −0.01691 | 1.0 | 0.952896 | 0 |
| hsa-miR-518c_st | −0.18337 | 1.1 | 0.329533 | 0 |
| hsa-miR-301a_st | 0.62109 | 1.5 | 0.091859 | 0 |
| hsa-miR-518d-3p_st | −0.12201 | 1.1 | 0.377787 | 0 |
| hsa-miR-518d-5p_st | 0.412508 | 1.3 | 0.058226 | 0 |
| hsa-miR-518e_st | −0.12906 | 1.1 | 0.470771 | 0 |
| hsa-let-7f_st | 0.385299 | 1.3 | 0.17797 | 0 |
| hsa-miR-518f_st | −0.15768 | 1.1 | 0.496288 | 0 |
| hsa-miR-1826_st | −0.28317 | 1.2 | 0.146806 | 0 |
| hsa-miR-519a_st | −0.3519 | 1.3 | 0.101156 | 0 |
| hsa-miR-519a-star_st | 0.205646 | 1.2 | 0.53008 | 0 |
| hsa-miR-519b-3p_st | −0.11703 | 1.1 | 0.618565 | 0 |
| hsa-miR-1283_st | −0.50359 | 1.4 | 0.004887 | 1 |
| hsa-miR-519c-3p_st | −0.36385 | 1.3 | 0.214256 | 0 |
| hsa-miR-519c-5p_st | 0.423196 | 1.3 | 0.133279 | 0 |
| hsa-miR-519d_st | −0.11132 | 1.1 | 0.446168 | 0 |
| hsa-miR-519e_st | −0.06322 | 1.0 | 0.78082 | 0 |
| hsa-miR-519e-star_st | −0.1214 | 1.1 | 0.46648 | 0 |
| hsa-miR-520a-3p_st | −0.0719 | 1.1 | 0.736346 | 0 |
| hsa-miR-1908_st | −0.20244 | 1.2 | 0.319725 | 0 |
| hsa-miR-520b_st | −0.08424 | 1.1 | 0.535892 | 0 |
| hsa-miR-520c-3p_st | 0.097327 | 1.1 | 0.626761 | 0 |
| hsa-miR-520c-5p_st | 0.214178 | 1.2 | 0.418918 | 0 |
| hsa-miR-181a-star_st | 0.586007 | 1.5 | 0.302362 | 0 |
| hsa-miR-21_st | 1.198378 | 2.3 | 0.073527 | 0 |
| hsa-miR-520e_st | −0.10278 | 1.1 | 0.642525 | 0 |
| hsa-miR-520f_st | 0.044265 | 1.0 | 0.845596 | 0 |
| hsa-miR-520g_st | −0.10602 | 1.1 | 0.684439 | 0 |
| hsa-miR-520h_st | 0.025191 | 1.0 | 0.872632 | 0 |
| hsa-miR-521_st | 0.135951 | 1.1 | 0.502529 | 0 |
| hsa-miR-302a-star_st | −0.51222 | 1.4 | 0.006825 | 1 |
| hsa-miR-886-3p_st | 0.757294 | 1.7 | 0.29616 | 0 |
| hsa-miR-523_st | −0.34897 | 1.3 | 0.127032 | 0 |
| hsa-miR-29a_st | 0.823801 | 1.8 | 0.027574 | 1 |
| hsa-miR-524-3p_st | −0.27697 | 1.2 | 0.056472 | 0 |
| hsa-miR-142-3p_st | −0.52651 | 1.4 | 0.002399 | 1 |
| hsa-miR-525-3p_st | −0.28289 | 1.2 | 0.20054 | 0 |
| hsa-miR-525-5p_st | 0.17017 | 1.1 | 0.507998 | 0 |
| hsa-miR-762_st | −0.22629 | 1.2 | 0.242063 | 0 |
| hsa-miR-423-3p_st | 0.594292 | 1.5 | 0.07215 | 0 |
| hsa-miR-449b-star_st | −0.53167 | 1.4 | 0.042588 | 1 |
| hsa-miR-527_st | −0.12407 | 1.1 | 0.677438 | 0 |
| hsa-miR-532-3p_st | 0.390028 | 1.3 | 0.325691 | 0 |
| hsa-miR-532-5p_st | 0.610915 | 1.5 | 0.129647 | 0 |
| hsa-miR-539_st | 0.237372 | 1.2 | 0.463201 | 0 |
| hsa-let-7a_st | 0.343599 | 1.3 | 0.160188 | 0 |
| hsa-miR-541-star_st | −0.22761 | 1.2 | 0.272677 | 0 |
| hsa-miR-542-3p_st | 0.085349 | 1.1 | 0.870538 | 0 |
| hsa-miR-542-5p_st | 0.318022 | 1.2 | 0.182103 | 0 |
| hsa-miR-543_st | 0.191655 | 1.1 | 0.575531 | 0 |
| hsa-miR-544_st | −0.18364 | 1.1 | 0.421129 | 0 |
| hsa-miR-544b_st | −0.18391 | 1.1 | 0.128518 | 0 |
| hsa-miR-545_st | −0.14921 | 1.1 | 0.511037 | 0 |
| hsa-miR-545-star_st | −0.10484 | 1.1 | 0.692111 | 0 |
| hsa-miR-548a-3p_st | −0.39676 | 1.3 | 0.138246 | 0 |
| hsa-miR-2861_st | −0.25298 | 1.2 | 0.201364 | 0 |
| hsa-miR-31_st | 0.823581 | 1.8 | 0.508736 | 0 |
| hsa-miR-367_st | −0.54016 | 1.5 | 0.039111 | 1 |
| hsa-miR-548c-3p_st | −0.228 | 1.2 | 0.435425 | 0 |
| hsa-miR-548c-5p_st | 0.192035 | 1.1 | 0.59889 | 0 |
| hsa-miR-548d-3p_st | −0.16537 | 1.1 | 0.354977 | 0 |
| hsa-miR-548d-5p_st | 0.07192 | 1.1 | 0.813325 | 0 |
| hsa-miR-548e_st | −0.11672 | 1.1 | 0.580217 | 0 |
| hsa-miR-548f_st | −0.10965 | 1.1 | 0.572805 | 0 |
| hsa-miR-548g_st | −0.20237 | 1.2 | 0.368203 | 0 |
| hsa-miR-548h_st | 0.316813 | 1.2 | 0.140388 | 0 |
| hsa-miR-548i_st | −0.24672 | 1.2 | 0.265634 | 0 |
| hsa-miR-548j_st | 0.040691 | 1.0 | 0.867893 | 0 |
| hsa-miR-548k_st | −0.07721 | 1.1 | 0.601183 | 0 |
| hsa-miR-526b-star_st | −0.54318 | 1.5 | 0.022687 | 1 |
| hsa-miR-548m_st | −0.02982 | 1.0 | 0.871167 | 0 |
| hsa-miR-548n_st | 0.039972 | 1.0 | 0.802876 | 0 |
| hsa-miR-548o_st | −0.24685 | 1.2 | 0.259895 | 0 |
| hsa-miR-548p_st | −0.16997 | 1.1 | 0.439908 | 0 |
| hsa-miR-548q_st | −0.25939 | 1.2 | 0.361052 | 0 |
| hsa-miR-548s_st | −0.19154 | 1.1 | 0.265269 | 0 |
| hsa-miR-548t_st | −0.31938 | 1.2 | 0.077064 | 0 |
| hsa-miR-548u_st | −0.04713 | 1.0 | 0.846127 | 0 |
| hsa-miR-638_st | −0.22643 | 1.2 | 0.284057 | 0 |
| hsa-miR-548w_st | 0.009834 | 1.0 | 0.979476 | 0 |
| hsa-miR-3119_st | −0.54399 | 1.5 | 0.029221 | 1 |
| hsa-miR-549_st | −0.29245 | 1.2 | 0.107155 | 0 |
| hsa-miR-205_st | −0.21745 | 1.2 | 0.85965 | 0 |
| hsa-miR-550-star_st | 0.549901 | 1.5 | 0.169503 | 0 |
| hsa-miR-551a_st | −0.35712 | 1.3 | 0.109667 | 0 |
| hsa-miR-93_st | 0.858992 | 1.8 | 0.030281 | 1 |
| hsa-miR-551b-star_st | 0.080113 | 1.1 | 0.715935 | 0 |
| hsa-let-7d-star_st | 0.262997 | 1.2 | 0.622517 | 0 |
| hsa-miR-553_st | 0.013205 | 1.0 | 0.944811 | 0 |
| hsa-miR-554_st | −0.10433 | 1.1 | 0.489795 | 0 |
| hsa-miR-555_st | −0.23948 | 1.2 | 0.23392 | 0 |
| hsa-miR-556-3p_st | −0.47354 | 1.4 | 0.056689 | 0 |
| hsa-miR-556-5p_st | −0.23992 | 1.2 | 0.172875 | 0 |
| hsa-miR-557_st | −0.16363 | 1.1 | 0.433601 | 0 |
| hsa-miR-558_st | −0.00478 | 1.0 | 0.979717 | 0 |
| hsa-miR-559_st | −0.15186 | 1.1 | 0.430198 | 0 |
| hsa-miR-561_st | 0.006506 | 1.0 | 0.974951 | 0 |
| hsa-miR-562_st | −0.21571 | 1.2 | 0.270021 | 0 |
| hsa-miR-563_st | −0.05815 | 1.0 | 0.80039 | 0 |
| hsa-miR-361-5p_st | 0.613429 | 1.5 | 0.08676 | 0 |
| hsa-miR-566_st | 0.046614 | 1.0 | 0.85342 | 0 |
| hsa-miR-567_st | −0.37537 | 1.3 | 0.210484 | 0 |
| hsa-miR-568_st | −0.21699 | 1.2 | 0.201307 | 0 |
| hsa-miR-29c_st | 0.871742 | 1.8 | 0.028813 | 1 |
| hsa-miR-570_st | −0.22875 | 1.2 | 0.116054 | 0 |
| hsa-miR-571_st | −0.26328 | 1.2 | 0.235531 | 0 |
| hsa-miR-572_st | −0.27599 | 1.2 | 0.130789 | 0 |
| hsa-miR-573_st | −0.21549 | 1.2 | 0.220353 | 0 |
| hsa-miR-132_st | 1.012765 | 2.0 | 0.11538 | 0 |
| hsa-miR-574-5p_st | 0.180112 | 1.1 | 0.38363 | 0 |
| hsa-let-7d_st | 0.390202 | 1.3 | 0.154499 | 0 |
| hsa-miR-576-3p_st | 0.072176 | 1.1 | 0.740656 | 0 |
| hsa-miR-576-5p_st | 0.022737 | 1.0 | 0.931348 | 0 |
| hsa-miR-577_st | −0.21436 | 1.2 | 0.32068 | 0 |
| hsa-miR-578_st | −0.28767 | 1.2 | 0.144755 | 0 |
| hsa-miR-579_st | −0.28676 | 1.2 | 0.054905 | 0 |
| hsa-miR-580_st | −0.22823 | 1.2 | 0.245572 | 0 |
| hsa-miR-191_st | 0.611596 | 1.5 | 0.100576 | 0 |
| hsa-miR-582-3p_st | −0.29882 | 1.2 | 0.173995 | 0 |
| hsa-miR-582-5p_st | −0.13183 | 1.1 | 0.739206 | 0 |
| hsa-miR-1915_st | −0.23195 | 1.2 | 0.277909 | 0 |
| hsa-let-7c_st | 0.41704 | 1.3 | 0.147112 | 0 |
| hsa-miR-585_st | −0.05815 | 1.0 | 0.756127 | 0 |
| hsa-miR-586_st | −0.20801 | 1.2 | 0.330797 | 0 |
| hsa-miR-324-5p_st | 0.911369 | 1.9 | 0.07229 | 0 |
| hsa-miR-588_st | −0.00721 | 1.0 | 0.972887 | 0 |
| hsa-miR-589_st | 0.261368 | 1.2 | 0.290756 | 0 |
| hsa-miR-589-star_st | −0.04948 | 1.0 | 0.827294 | 0 |
| hsa-miR-188-5p_st | −0.57728 | 1.5 | 0.029955 | 1 |
| hsa-miR-103_st | 0.736177 | 1.7 | 0.045179 | 1 |
| hsa-miR-591_st | −0.09858 | 1.1 | 0.637415 | 0 |
| hsa-miR-592_st | 0.201385 | 1.1 | 0.433659 | 0 |
| hsa-miR-593_st | −0.29518 | 1.2 | 0.252382 | 0 |
| hsa-miR-23b_st | 0.498592 | 1.4 | 0.088324 | 0 |
| hsa-miR-595_st | −0.26424 | 1.2 | 0.132634 | 0 |
| hsa-miR-99a_st | 0.267713 | 1.2 | 0.588923 | 0 |
| hsa-miR-15b_st | 0.88722 | 1.8 | 0.09237 | 0 |
| hsa-miR-135b_st | 0.067593 | 1.0 | 0.890162 | 0 |
| hsa-miR-599_st | −0.18698 | 1.1 | 0.444202 | 0 |
| hsa-miR-600_st | −0.20556 | 1.2 | 0.580542 | 0 |
| hsa-miR-107_st | 0.69593 | 1.6 | 0.041501 | 1 |
| hsa-miR-602_st | −0.03475 | 1.0 | 0.844066 | 0 |
| hsa-miR-603_st | −0.1608 | 1.1 | 0.284283 | 0 |
| hsa-miR-3065-5p_st | 0.144608 | 1.1 | 0.792882 | 0 |
| hsa-miR-605_st | −0.39692 | 1.3 | 0.184109 | 0 |
| hsa-miR-24_st | 0.697423 | 1.6 | 0.044964 | 1 |
| hsa-miR-607_st | −0.05247 | 1.0 | 0.736567 | 0 |
| hsa-miR-608_st | −0.10417 | 1.1 | 0.604513 | 0 |
| hsa-miR-609_st | 0.186243 | 1.1 | 0.343471 | 0 |
| hsa-miR-3196_st | −0.23185 | 1.2 | 0.279359 | 0 |
| hsa-miR-26b_st | 0.899369 | 1.9 | 0.052113 | 0 |
| hsa-miR-28-5p_st | 0.720874 | 1.6 | 0.018164 | 1 |
| hsa-miR-613_st | −0.14196 | 1.1 | 0.478472 | 0 |
| hsa-miR-338-3p_st | 0.935617 | 1.9 | 0.127168 | 0 |
| hsa-miR-615-3p_st | −0.08947 | 1.1 | 0.725218 | 0 |
| hsa-miR-454_st | 1.45012 | 2.7 | 0.012139 | 1 |
| hsa-miR-616_st | 0.364132 | 1.3 | 0.119188 | 0 |
| hsa-miR-151-5p_st | 0.644212 | 1.6 | 0.028287 | 1 |
| hsa-miR-617_st | −0.173 | 1.1 | 0.675479 | 0 |
| hsa-miR-128_st | 1.48222 | 2.8 | 0.004492 | 1 |
| hsa-miR-619_st | −0.1761 | 1.1 | 0.272833 | 0 |
| hsa-miR-620_st | −0.31701 | 1.2 | 0.179873 | 0 |
| hsa-miR-621_st | 0.060822 | 1.0 | 0.753572 | 0 |
| hsa-let-7b_st | 0.496653 | 1.4 | 0.228383 | 0 |
| hsa-miR-623_st | −0.13342 | 1.1 | 0.289272 | 0 |
| hsa-miR-624_st | −0.24918 | 1.2 | 0.080305 | 0 |
| hsa-miR-624-star_st | −0.04709 | 1.0 | 0.865835 | 0 |
| hsa-miR-183_st | 0.749486 | 1.7 | 0.209996 | 0 |
| hsa-miR-30b_st | 0.635057 | 1.6 | 0.030704 | 1 |
| hsa-miR-626_st | −0.30024 | 1.2 | 0.084686 | 0 |
| hsa-miR-627_st | −0.27185 | 1.2 | 0.166404 | 0 |
| hsa-miR-628-3p_st | −0.45608 | 1.4 | 0.352013 | 0 |
| hsa-miR-31-star_st | 0.556685 | 1.5 | 0.627062 | 0 |
| hsa-miR-218-1-star_st | −0.57755 | 1.5 | 0.02377 | 1 |
| hsa-miR-629-star_st | 0.608438 | 1.5 | 0.054473 | 0 |
| hsa-miR-630_st | −0.43884 | 1.4 | 0.220528 | 0 |
| hsa-miR-631_st | −0.3752 | 1.3 | 0.337392 | 0 |
| hsa-miR-632_st | −0.35562 | 1.3 | 0.197197 | 0 |
| hsa-miR-633_st | −0.27032 | 1.2 | 0.130635 | 0 |
| hsa-miR-634_st | −0.45522 | 1.4 | 0.376858 | 0 |
| hsa-miR-635_st | −0.27473 | 1.2 | 0.285299 | 0 |
| hsa-miR-203_st | 0.694715 | 1.6 | 0.473822 | 0 |
| hsa-miR-331-3p_st | 0.274415 | 1.2 | 0.379522 | 0 |
| hsa-miR-125b_st | 0.344383 | 1.3 | 0.18163 | 0 |
| hsa-let-7i_st | 0.231105 | 1.2 | 0.534606 | 0 |
| hsa-miR-640_st | −0.0665 | 1.0 | 0.754923 | 0 |
| hsa-miR-641_st | 0.155287 | 1.1 | 0.561397 | 0 |
| hsa-miR-642_st | −0.15477 | 1.1 | 0.348065 | 0 |
| hsa-miR-29b_st | 1.07442 | 2.1 | 0.035492 | 1 |
| hsa-miR-122_st | −0.58419 | 1.5 | 0.019978 | 1 |
| hsa-miR-645_st | 0.148649 | 1.1 | 0.342359 | 0 |
| hsa-miR-646_st | 0.116662 | 1.1 | 0.57264 | 0 |
| hsa-miR-647_st | −0.27979 | 1.2 | 0.185186 | 0 |
| hsa-miR-648_st | −0.22064 | 1.2 | 0.223533 | 0 |
| hsa-miR-649_st | −0.05432 | 1.0 | 0.844278 | 0 |
| hsa-miR-182_st | 0.976855 | 2.0 | 0.080887 | 0 |
| hsa-miR-651_st | −0.27723 | 1.2 | 0.134973 | 0 |
| hsa-miR-652_st | 0.13121 | 1.1 | 0.676776 | 0 |
| hsa-miR-653_st | −0.24667 | 1.2 | 0.2497 | 0 |
| hsa-miR-654-3p_st | 0.363983 | 1.3 | 0.539926 | 0 |
| hsa-miR-374b_st | 1.127673 | 2.2 | 0.066007 | 0 |
| hsa-miR-130a_st | 1.332063 | 2.5 | 0.002319 | 1 |
| hsa-miR-656_st | −0.36889 | 1.3 | 0.099742 | 0 |
| hsa-miR-657_st | −0.01674 | 1.0 | 0.933805 | 0 |
| hsa-miR-658_st | 0.139531 | 1.1 | 0.528627 | 0 |
| hsa-miR-99b_st | 0.678469 | 1.6 | 0.063338 | 0 |
| hsa-miR-660_st | 0.941285 | 1.9 | 0.109644 | 0 |
| hsa-miR-661_st | −0.41538 | 1.3 | 0.064604 | 0 |
| hsa-miR-30e-star_st | 0.69883 | 1.6 | 0.172264 | 0 |
| hsa-miR-663_st | −0.20328 | 1.2 | 0.251363 | 0 |
| hsa-miR-663b_st | −0.23182 | 1.2 | 0.29588 | 0 |
| hsa-miR-664_st | 0.045638 | 1.0 | 0.912427 | 0 |
| hsa-miR-664-star_st | −0.47706 | 1.4 | 0.139167 | 0 |
| hsa-miR-30d_st | 0.558599 | 1.5 | 0.037068 | 1 |
| hsa-miR-668_st | 0.221637 | 1.2 | 0.39578 | 0 |
| hsa-miR-670_st | −0.27934 | 1.2 | 0.255478 | 0 |
| hsa-miR-671-3p_st | 0.419363 | 1.3 | 0.14201 | 0 |
| hsa-miR-671-5p_st | −0.17283 | 1.1 | 0.308152 | 0 |
| hsa-miR-30a-star_st | 0.845819 | 1.8 | 0.109974 | 0 |
| hsa-miR-675-star_st | 0.192926 | 1.1 | 0.454078 | 0 |
| hsa-miR-148b_st | 0.542358 | 1.5 | 0.15977 | 0 |
| hsa-miR-708_st | 0.022573 | 1.0 | 0.976157 | 0 |
| hsa-miR-708-star_st | −0.20003 | 1.1 | 0.389326 | 0 |
| hsa-miR-221-star_st | 0.644389 | 1.6 | 0.443095 | 0 |
| hsa-let-7e_st | 0.602002 | 1.5 | 0.079492 | 0 |
| hsa-miR-7-1-star_st | 0.818186 | 1.8 | 0.090888 | 0 |
| hsa-miR-27b_st | 0.760861 | 1.7 | 0.050089 | 0 |
| hsa-miR-34a_st | 0.383241 | 1.3 | 0.56035 | 0 |
| hsa-miR-744_st | −0.22482 | 1.2 | 0.281118 | 0 |
| hsa-miR-744-star_st | 0.000885 | 1.0 | 0.997387 | 0 |
| hsa-miR-92b_st | 0.969394 | 2.0 | 0.100133 | 0 |
| hsa-miR-759_st | 0.047893 | 1.0 | 0.80925 | 0 |
| hsa-miR-760_st | −0.032 | 1.0 | 0.878359 | 0 |
| hsa-miR-92a_st | 0.958113 | 1.9 | 0.050394 | 0 |
| hsa-miR-335_st | 1.148293 | 2.2 | 0.049604 | 1 |
| hsa-miR-96_st | 1.370267 | 2.6 | 0.030038 | 1 |
| hsa-miR-95_st | 0.75024 | 1.7 | 0.191291 | 0 |
| hsa-miR-766_st | 0.094881 | 1.1 | 0.619106 | 0 |
| hsa-miR-767-3p_st | −0.15334 | 1.1 | 0.435771 | 0 |
| hsa-miR-767-5p_st | 0.074607 | 1.1 | 0.698643 | 0 |
| hsa-miR-98_st | 0.793304 | 1.7 | 0.086422 | 0 |
| hsa-miR-769-5p_st | 0.404622 | 1.3 | 0.274484 | 0 |
| hsa-miR-770-5p_st | 0.252234 | 1.2 | 0.327362 | 0 |
| hsa-miR-802_st | −0.25723 | 1.2 | 0.226403 | 0 |
| hsa-miR-34b-star_st | 0.667378 | 1.6 | 0.222506 | 0 |
| hsa-miR-581_st | −0.5927 | 1.5 | 0.043457 | 1 |
| hsa-miR-181d_st | 0.726291 | 1.7 | 0.05447 | 0 |
| hsa-miR-875-5p_st | −0.08774 | 1.1 | 0.713419 | 0 |
| hsa-miR-876-3p_st | −0.01905 | 1.0 | 0.922733 | 0 |
| hsa-miR-876-5p_st | −0.35423 | 1.3 | 0.104072 | 0 |
| hsa-miR-151-3p_st | 0.967325 | 2.0 | 0.004102 | 1 |
| hsa-miR-877-star_st | −0.24862 | 1.2 | 0.411266 | 0 |
| hsa-miR-885-3p_st | −0.01471 | 1.0 | 0.950795 | 0 |
| hsa-miR-200c_st | 0.351142 | 1.3 | 0.162451 | 0 |
| hsa-miR-429_st | 0.614115 | 1.5 | 0.431716 | 0 |
| hsa-miR-886-5p_st | 0.28086 | 1.2 | 0.449666 | 0 |
| hsa-miR-224_st | 1.227236 | 2.3 | 0.0576 | 0 |
| hsa-miR-888_st | −0.01071 | 1.0 | 0.960366 | 0 |
| hsa-miR-4321_st | −0.6052 | 1.5 | 0.030217 | 1 |
| hsa-miR-889_st | −0.23227 | 1.2 | 0.224312 | 0 |
| hsa-miR-181b_st | 0.829098 | 1.8 | 0.038702 | 1 |
| hsa-miR-891a_st | 0.052375 | 1.0 | 0.871011 | 0 |
| hsa-miR-891b_st | −0.06124 | 1.0 | 0.758825 | 0 |
| hsa-miR-892a_st | −0.29058 | 1.2 | 0.402913 | 0 |
| hsa-miR-892b_st | −0.0824 | 1.1 | 0.72864 | 0 |
| hsa-miR-9_st | −0.10667 | 1.1 | 0.531681 | 0 |
| hsa-miR-222_st | 0.856482 | 1.8 | 0.356972 | 0 |
| hsa-miR-141_st | 1.025937 | 2.0 | 0.03662 | 1 |
| hsa-miR-221_st | 0.75331 | 1.7 | 0.391953 | 0 |
| hsa-miR-924_st | −0.05225 | 1.0 | 0.699682 | 0 |
| hsa-miR-181a_st | 0.660999 | 1.6 | 0.199088 | 0 |
| hsa-miR-181c_st | 0.693852 | 1.6 | 0.214097 | 0 |
| hsa-miR-4324_st | 0.694992 | 1.6 | 0.153322 | 0 |
| hsa-miR-125a-5p_st | 0.613669 | 1.5 | 0.147431 | 0 |
| hsa-miR-92b-star_st | −0.15828 | 1.1 | 0.338215 | 0 |
| hsa-miR-3195_st | −0.62112 | 1.5 | 0.030017 | 1 |
| hsa-miR-3137_st | −0.67262 | 1.6 | 0.012909 | 1 |
| hsa-miR-934_st | −0.12305 | 1.1 | 0.606649 | 0 |
| hsa-miR-935_st | 0.061615 | 1.0 | 0.824991 | 0 |
| hsa-miR-551b_st | 0.829046 | 1.8 | 0.377235 | 0 |
| hsa-miR-937_st | −0.24289 | 1.2 | 0.445653 | 0 |
| hsa-miR-938_st | −0.04506 | 1.0 | 0.828648 | 0 |
| hsa-miR-939_st | −0.19957 | 1.1 | 0.198377 | 0 |
| hsa-miR-7-2-star_st | −0.95235 | 1.9 | 0.246207 | 0 |
| hsa-miR-146b-5p_st | 2.170586 | 4.5 | 0.035006 | 1 |
| hsa-miR-941_st | 0.024869 | 1.0 | 0.905811 | 0 |
| hsa-miR-942_st | −0.20163 | 1.1 | 0.336643 | 0 |
| hsa-miR-200a_st | 0.39401 | 1.3 | 0.696049 | 0 |
| hsa-miR-944_st | −0.1237 | 1.1 | 0.599987 | 0 |
| hsa-miR-7_st | −1.71247 | 3.3 | 0.24874 | 0 |
| hsa-miR-375_st | 0.857487 | 1.8 | 0.046519 | 1 |
| hsa-miR-96-star_st | 0.083458 | 1.1 | 0.719748 | 0 |
| hsa-miR-424_st | 0.955879 | 1.9 | 0.151543 | 0 |
| hsa-miR-181a-2-star_st | 1.717123 | 3.3 | 0.003546 | 1 |
| hsa-miR-200b_st | 0.202495 | 1.2 | 0.83073 | 0 |
| hsa-miR-146b-3p_st | 0.976691 | 2.0 | 0.181724 | 0 |
| hsa-miR-99b-star_st | −0.01575 | 1.0 | 0.943836 | 0 |
| hsa-miR-9-star_st | 0.025966 | 1.0 | 0.930189 | 0 |

| | | Fold change | | |
|---|---|---|---|---|
| | Log2Diff (Hash | (Hash vs | Ttest (Hash vs | |
| Sample Name | vs Benign) | Benign) | Benign) | p < 0.05 |
| hsa-miR-150_st | 2.203453771 | 4.6 | 0.008364 | 1 |
| hsa-let-7a-2-star_st | 0.011609636 | 1.0 | 0.74618 | 0 |
| hsa-let-7a-star_st | −0.231021394 | 1.2 | 0.489092 | 0 |
| hsa-miR-513b_st | 0.692991032 | 1.6 | 0.10349 | 0 |
| hsa-let-7b-star_st | −0.113920071 | 1.1 | 0.521436 | 0 |
| hsa-miR-593-star_st | 0.666203761 | 1.6 | 0.111667 | 0 |
| hsa-let-7c-star_st | 0.224440211 | 1.2 | 0.083203 | 0 |
| hsa-miR-516a-5p_st | 0.753374103 | 1.7 | 0.113246 | 0 |
| hsa-miR-142-5p_st | 2.56169858 | 5.9 | 0.013961 | 1 |
| hsa-miR-650_st | 0.730169717 | 1.7 | 0.010038 | 1 |
| hsa-miR-513c_st | 0.605038869 | 1.5 | 0.166732 | 0 |
| hsa-miR-761_st | 0.777426676 | 1.7 | 0.05169 | 0 |
| hsa-let-7f-1-star_st | 0.142846126 | 1.1 | 0.415137 | 0 |
| hsa-miR-513a-5p_st | 0.523435037 | 1.4 | 0.198806 | 0 |
| hsa-miR-654-5p_st | 0.249276505 | 1.2 | 0.360797 | 0 |
| hsa-let-7g-star_st | 0.156253707 | 1.1 | 0.148959 | 0 |
| hsa-miR-3163_st | 0.543188999 | 1.5 | 0.02872 | 1 |
| hsa-let-7i-star_st | 0.130185751 | 1.1 | 0.670973 | 0 |
| hsa-miR-3165_st | 0.43205265 | 1.3 | 0.16596 | 0 |
| hsa-miR-3194_st | 0.429406799 | 1.3 | 0.103363 | 0 |
| hsa-miR-100-star_st | −0.195570551 | 1.1 | 0.235634 | 0 |
| hsa-miR-4251_st | 0.578591196 | 1.5 | 0.132532 | 0 |
| hsa-miR-101-star_st | 0.012914493 | 1.0 | 0.960248 | 0 |
| hsa-miR-922_st | 0.711925981 | 1.6 | 0.014799 | 1 |
| hsa-miR-103-2-star_st | 0.350606284 | 1.3 | 0.253605 | 0 |
| hsa-miR-155_st | 1.798436129 | 3.5 | 0.014737 | 1 |
| hsa-miR-105_st | −0.233692782 | 1.2 | 0.442062 | 0 |
| hsa-miR-127-5p_st | 0.642003592 | 1.6 | 0.090898 | 0 |
| hsa-miR-1286_st | 0.743468778 | 1.7 | 0.05942 | 0 |
| hsa-miR-106a-star_st | −0.184227718 | 1.1 | 0.368139 | 0 |
| hsa-miR-890_st | 0.909456805 | 1.9 | 0.012924 | 1 |
| hsa-miR-3126-5p_st | 0.226396632 | 1.2 | 0.246346 | 0 |
| hsa-miR-489_st | 0.498037741 | 1.4 | 0.242395 | 0 |
| hsa-miR-10a_st | 0.030676675 | 1.0 | 0.886626 | 0 |
| hsa-miR-10a-star_st | 0.174697763 | 1.1 | 0.615306 | 0 |
| hsa-miR-10b_st | 0.047827215 | 1.0 | 0.779194 | 0 |
| hsa-miR-10b-star_st | 0.341931915 | 1.3 | 0.184372 | 0 |
| hsa-miR-1178_st | 0.047470858 | 1.0 | 0.769634 | 0 |
| hsa-miR-1179_st | −0.090584137 | 1.1 | 0.754225 | 0 |
| hsa-miR-1180_st | −0.31074378 | 1.2 | 0.601011 | 0 |
| hsa-miR-1181_st | 0.614349339 | 1.5 | 0.298981 | 0 |
| hsa-miR-3138_st | 0.278810901 | 1.2 | 0.408688 | 0 |
| hsa-miR-220c_st | 0.629203111 | 1.5 | 0.059606 | 0 |
| hsa-miR-510_st | 0.316980656 | 1.2 | 0.117429 | 0 |
| hsa-miR-1185_st | −0.128048893 | 1.1 | 0.639716 | 0 |
| hsa-miR-1193_st | −0.103716351 | 1.1 | 0.523278 | 0 |
| hsa-miR-1197_st | −0.310732889 | 1.2 | 0.078067 | 0 |
| hsa-miR-1200_st | 0.223603575 | 1.2 | 0.39149 | 0 |
| hsa-miR-1201_st | −0.075320777 | 1.1 | 0.227089 | 0 |
| hsa-miR-1202_st | −0.112431833 | 1.1 | 0.486393 | 0 |
| hsa-miR-30c-1-star_st | 0.380452272 | 1.3 | 0.065707 | 0 |
| hsa-miR-1204_st | 0.222358878 | 1.2 | 0.220054 | 0 |
| hsa-miR-1205_st | 0.33892738 | 1.3 | 0.272226 | 0 |
| hsa-miR-1206_st | −0.153352729 | 1.1 | 0.835432 | 0 |
| hsa-miR-1207-3p_st | −0.092422989 | 1.1 | 0.761077 | 0 |
| hsa-miR-1207-5p_st | −0.241697006 | 1.2 | 0.345079 | 0 |
| hsa-miR-1208_st | −0.177068911 | 1.1 | 0.912285 | 0 |
| hsa-miR-622_st | 0.538457109 | 1.5 | 0.010469 | 1 |
| hsa-miR-1224-3p_st | 0.187520742 | 1.1 | 0.446743 | 0 |
| hsa-miR-2116_st | 0.492500977 | 1.4 | 0.117165 | 0 |
| hsa-miR-1225-3p_st | 0.100043389 | 1.1 | 0.376665 | 0 |
| hsa-miR-1225-5p_st | −0.216538721 | 1.2 | 0.326366 | 0 |
| hsa-miR-1226_st | 0.05345221 | 1.0 | 0.63599 | 0 |
| hsa-miR-30b-star_st | 0.378859796 | 1.3 | 0.024537 | 1 |
| hsa-miR-1227_st | 0.177934835 | 1.1 | 0.235217 | 0 |
| hsa-miR-1295_st | 0.476249392 | 1.4 | 0.136194 | 0 |
| hsa-miR-1228-star_st | −0.460203769 | 1.4 | 0.135239 | 0 |
| hsa-miR-20b_st | 0.314717878 | 1.2 | 0.59429 | 0 |
| hsa-miR-122-star_st | −0.18321932 | 1.1 | 0.53654 | 0 |
| hsa-miR-1231_st | −0.359637397 | 1.3 | 0.147486 | 0 |
| hsa-miR-1233_st | −0.053097982 | 1.0 | 0.769955 | 0 |
| hsa-miR-524-5p_st | 0.799646437 | 1.7 | 0.020832 | 1 |
| hsa-miR-1236_st | 0.380144023 | 1.3 | 0.45589 | 0 |
| hsa-miR-873_st | 0.639177977 | 1.6 | 0.02933 | 1 |
| hsa-miR-1238_st | 0.14455622 | 1.1 | 0.308167 | 0 |
| hsa-miR-124_st | 0.115237657 | 1.1 | 0.443001 | 0 |
| hsa-miR-1243_st | 0.182963403 | 1.1 | 0.10554 | 0 |
| hsa-miR-1244_st | 0.442283194 | 1.4 | 0.28315 | 0 |
| hsa-miR-1245_st | −0.071613502 | 1.1 | 0.603549 | 0 |
| hsa-miR-3122_st | 0.136397635 | 1.1 | 0.407173 | 0 |
| hsa-miR-134_st | 0.182840295 | 1.1 | 0.537802 | 0 |
| hsa-miR-1248_st | 0.386913244 | 1.3 | 0.771592 | 0 |
| hsa-miR-146a_st | 1.721943675 | 3.3 | 0.046042 | 1 |
| hsa-miR-124-star_st | −0.036904116 | 1.0 | 0.526467 | 0 |
| hsa-miR-1250_st | −0.111162351 | 1.1 | 0.87748 | 0 |
| hsa-miR-764_st | 0.627906523 | 1.5 | 0.06263 | 0 |
| hsa-miR-1252_st | −0.17930367 | 1.1 | 0.21923 | 0 |
| hsa-miR-223_st | 1.450273001 | 2.7 | 0.020339 | 1 |
| hsa-miR-34c-3p_st | 0.359030798 | 1.3 | 0.43261 | 0 |
| hsa-miR-1255a_st | 0.644904909 | 1.6 | 0.1947 | 0 |
| hsa-miR-1255b_st | 0.206778521 | 1.2 | 0.943722 | 0 |
| hsa-miR-1256_st | −0.038585498 | 1.0 | 0.528789 | 0 |
| hsa-miR-1257_st | 0.483313452 | 1.4 | 0.094918 | 0 |
| hsa-miR-199b-5p_st | 1.374135983 | 2.6 | 0.002211 | 1 |
| hsa-miR-1259_st | −0.416334147 | 1.3 | 0.145578 | 0 |
| hsa-miR-492_st | 0.330597852 | 1.3 | 0.222847 | 0 |
| hsa-miR-3132_st | 0.376146181 | 1.3 | 0.084348 | 0 |
| hsa-miR-516b_st | 0.389547759 | 1.3 | 0.160611 | 0 |
| hsa-miR-4294_st | 0.267665335 | 1.2 | 0.484447 | 0 |
| hsa-miR-125b-2-star_st | 0.570600914 | 1.5 | 0.501618 | 0 |
| hsa-miR-126_st | −0.291752851 | 1.2 | 0.226422 | 0 |
| hsa-miR-198_st | 0.216067303 | 1.2 | 0.488113 | 0 |
| hsa-miR-3191_st | 0.428319564 | 1.3 | 0.273366 | 0 |
| hsa-miR-1261_st | 0.339073131 | 1.3 | 0.250594 | 0 |
| hsa-miR-1262_st | 0.246582458 | 1.2 | 0.237055 | 0 |
| hsa-miR-1263_st | 0.002994286 | 1.0 | 0.98887 | 0 |
| hsa-miR-1264_st | 0.129685159 | 1.1 | 0.504958 | 0 |
| hsa-miR-1265_st | −0.00478625 | 1.0 | 0.978527 | 0 |
| hsa-miR-2114-star_st | 0.434370937 | 1.4 | 0.590149 | 0 |
| hsa-miR-1267_st | −0.052505392 | 1.0 | 0.686661 | 0 |
| hsa-miR-1268_st | −0.311544831 | 1.2 | 0.395279 | 0 |
| hsa-miR-1269_st | 0.362377856 | 1.3 | 0.303275 | 0 |
| hsa-miR-509-5p_st | 0.475655138 | 1.4 | 0.128606 | 0 |
| hsa-miR-1270_st | −0.050719434 | 1.0 | 0.97721 | 0 |
| hsa-miR-1271_st | 0.271446664 | 1.2 | 0.161724 | 0 |
| hsa-miR-4303_st | 0.412391073 | 1.3 | 0.078033 | 0 |
| hsa-miR-1273_st | 0.165442504 | 1.1 | 0.947419 | 0 |
| hsa-miR-583_st | 0.687979689 | 1.6 | 0.027615 | 1 |
| hsa-miR-1273d_st | −0.013204854 | 1.0 | 0.906886 | 0 |
| hsa-miR-127-3p_st | 0.410577043 | 1.3 | 0.115024 | 0 |
| hsa-miR-522-star_st | 0.314980137 | 1.2 | 0.179915 | 0 |
| hsa-miR-214-star_st | 0.851687647 | 1.8 | 0.022454 | 1 |
| hsa-miR-184_st | 0.359357824 | 1.3 | 0.312376 | 0 |
| hsa-miR-614_st | 0.598523825 | 1.5 | 0.140224 | 0 |
| hsa-miR-526b_st | 0.521217217 | 1.4 | 0.097793 | 0 |
| hsa-miR-1277_st | −0.170162396 | 1.1 | 0.40409 | 0 |
| hsa-miR-1278_st | −0.172305953 | 1.1 | 0.462221 | 0 |
| hsa-miR-1279_st | 0.004626172 | 1.0 | 0.681663 | 0 |
| hsa-miR-409-3p_st | 0.768490808 | 1.7 | 0.000866 | 1 |
| hsa-miR-199a-5p_st | 0.827921846 | 1.8 | 0.0351 | 1 |
| hsa-miR-1281_st | 0.18164288 | 1.1 | 0.646935 | 0 |
| hsa-miR-1282_st | 0.623893244 | 1.5 | 0.205007 | 0 |
| hsa-miR-33b-star_st | 0.341258038 | 1.3 | 0.036234 | 1 |
| hsa-miR-1284_st | 0.501884007 | 1.4 | 0.299586 | 0 |
| hsa-miR-1253_st | 0.669248696 | 1.6 | 0.017339 | 1 |
| hsa-miR-584_st | 0.326999523 | 1.3 | 0.297353 | 0 |
| hsa-miR-1287_st | −0.015401509 | 1.0 | 0.553083 | 0 |
| hsa-miR-15a_st | 0.511485641 | 1.4 | 0.982591 | 0 |
| hsa-miR-1289_st | 0.041351958 | 1.0 | 0.855474 | 0 |
| hsa-miR-1290_st | −0.050188417 | 1.0 | 0.929735 | 0 |
| hsa-miR-2117_st | 0.506400174 | 1.4 | 0.043604 | 1 |
| hsa-miR-1292_st | −0.050843006 | 1.0 | 0.88824 | 0 |
| hsa-miR-1293_st | −0.073272405 | 1.1 | 0.739425 | 0 |
| hsa-miR-129-3p_st | −0.001693057 | 1.0 | 0.55594 | 0 |
| hsa-miR-520d-5p_st | 0.467552588 | 1.4 | 0.085284 | 0 |
| hsa-miR-526a_st | 0.578185042 | 1.5 | 0.028716 | 1 |
| hsa-miR-129-5p_st | −0.169948256 | 1.1 | 0.65617 | 0 |
| hsa-miR-1254_st | 0.148559927 | 1.1 | 0.439701 | 0 |
| hsa-miR-1297_st | −0.099944387 | 1.1 | 0.572545 | 0 |
| hsa-miR-1298_st | 0.539135199 | 1.5 | 0.182728 | 0 |
| hsa-miR-1299_st | 0.334121739 | 1.3 | 0.637893 | 0 |
| hsa-miR-129-star_st | 0.008650809 | 1.0 | 0.924159 | 0 |
| hsa-miR-1301_st | −0.02689071 | 1.0 | 0.694778 | 0 |
| hsa-miR-19b_st | 0.409106317 | 1.3 | 0.809645 | 0 |
| hsa-miR-1303_st | −0.162155607 | 1.1 | 0.903812 | 0 |
| hsa-miR-1304_st | 0.028103052 | 1.0 | 0.647933 | 0 |
| hsa-miR-1305_st | 0.294612715 | 1.2 | 0.585706 | 0 |
| hsa-miR-550_st | 0.366212669 | 1.3 | 0.263279 | 0 |
| hsa-miR-1307_st | −0.341057611 | 1.3 | 0.546509 | 0 |
| hsa-miR-1308_st | −0.069935135 | 1.0 | 0.130856 | 0 |
| hsa-miR-4291_st | 0.754340375 | 1.7 | 0.039109 | 1 |
| hsa-miR-130a-star_st | 0.028881865 | 1.0 | 0.691859 | 0 |
| hsa-miR-130b_st | 0.07684248 | 1.1 | 0.418256 | 0 |
| hsa-miR-130b-star_st | 0.093913981 | 1.1 | 0.277798 | 0 |
| hsa-miR-921_st | 0.195608523 | 1.1 | 0.445453 | 0 |
| hsa-miR-520a-5p_st | 0.576719796 | 1.5 | 0.007799 | 1 |
| hsa-miR-711_st | 0.260675292 | 1.2 | 0.321107 | 0 |
| hsa-miR-1323_st | 0.035822018 | 1.0 | 0.943835 | 0 |
| hsa-miR-1324_st | −0.13301591 | 1.1 | 0.627783 | 0 |
| hsa-miR-490-3p_st | 0.424119864 | 1.3 | 0.064365 | 0 |
| hsa-miR-718_st | 0.217732235 | 1.2 | 0.248671 | 0 |
| hsa-miR-133b_st | −0.469788265 | 1.4 | 0.917947 | 0 |
| hsa-miR-431-star_st | 0.147541588 | 1.1 | 0.018985 | 1 |
| hsa-miR-552_st | 0.343030797 | 1.3 | 0.445382 | 0 |
| hsa-miR-28-3p_st | 0.155071657 | 1.1 | 0.429667 | 0 |
| hsa-miR-4265_st | 0.152783398 | 1.1 | 0.763516 | 0 |
| hsa-miR-643_st | 0.503036953 | 1.4 | 0.071571 | 0 |
| hsa-miR-136_st | −0.20070054 | 1.1 | 0.374108 | 0 |
| hsa-miR-136-star_st | −0.064309513 | 1.0 | 0.452847 | 0 |
| hsa-miR-137_st | −0.260427174 | 1.2 | 0.097396 | 0 |
| hsa-miR-138_st | −1.084571389 | 2.1 | 0.076191 | 0 |
| hsa-miR-138-1-star_st | −0.107578237 | 1.1 | 0.735396 | 0 |
| hsa-miR-138-2-star_st | −0.115910616 | 1.1 | 0.471397 | 0 |
| hsa-miR-139-3p_st | 0.011765898 | 1.0 | 0.369208 | 0 |
| hsa-miR-139-5p_st | −0.948639603 | 1.9 | 0.148973 | 0 |
| hsa-miR-140-3p_st | 0.2619809 | 1.2 | 0.387848 | 0 |
| hsa-miR-140-5p_st | 0.682458919 | 1.6 | 0.513297 | 0 |
| hsa-miR-381_st | 0.571360316 | 1.5 | 0.041189 | 1 |
| hsa-miR-141-star_st | −0.022997755 | 1.0 | 0.773926 | 0 |
| hsa-miR-17-star_st | 0.296730861 | 1.2 | 0.069551 | 0 |
| hsa-miR-4300_st | 0.322573125 | 1.3 | 0.046109 | 1 |
| hsa-miR-143_st | −0.184341742 | 1.1 | 0.429635 | 0 |
| hsa-miR-143-star_st | −0.387219308 | 1.3 | 0.14521 | 0 |
| hsa-miR-144_st | −0.341966598 | 1.3 | 0.296928 | 0 |
| hsa-miR-3198_st | 0.164296782 | 1.1 | 0.241836 | 0 |
| hsa-miR-145_st | −0.682094867 | 1.6 | 0.162888 | 0 |
| hsa-miR-145-star_st | −0.163485512 | 1.1 | 0.66014 | 0 |
| hsa-miR-1468_st | 0.140185404 | 1.1 | 0.745272 | 0 |
| hsa-miR-1469_st | −0.563957441 | 1.5 | 0.051315 | 0 |
| hsa-miR-1973_st | 0.19433018 | 1.1 | 0.189619 | 0 |
| hsa-miR-3154_st | 0.179552967 | 1.1 | 0.248993 | 0 |
| hsa-miR-148a-star_st | 0.902934693 | 1.9 | 0.022192 | 1 |
| hsa-miR-92a-1-star_st | 0.132762782 | 1.1 | 0.196171 | 0 |
| hsa-miR-147_st | −0.085333922 | 1.1 | 0.953812 | 0 |
| hsa-miR-194-star_st | 0.145575377 | 1.1 | 0.348301 | 0 |
| hsa-miR-1471_st | 0.22006954 | 1.2 | 0.553971 | 0 |
| hsa-miR-147b_st | 0.147795482 | 1.1 | 0.156315 | 0 |
| hsa-miR-148a_st | 0.095586754 | 1.1 | 0.239099 | 0 |
| hsa-miR-4299_st | 0.247225958 | 1.2 | 0.588211 | 0 |
| hsa-miR-135a-star_st | 0.094731411 | 1.1 | 0.56718 | 0 |
| hsa-miR-612_st | 0.14154239 | 1.1 | 0.246254 | 0 |
| hsa-miR-1321_st | 0.292069649 | 1.2 | 0.132047 | 0 |
| hsa-miR-149-star_st | −0.496528448 | 1.4 | 0.069502 | 0 |
| hsa-miR-604_st | 0.368201664 | 1.3 | 0.602615 | 0 |
| hsa-miR-150-star_st | −0.160760929 | 1.1 | 0.380117 | 0 |
| hsa-miR-125b-1-star_st | 0.266214617 | 1.2 | 0.174494 | 0 |
| hsa-miR-1183_st | 0.172022645 | 1.1 | 0.286923 | 0 |
| hsa-miR-152_st | −0.634853536 | 1.6 | 0.131429 | 0 |
| hsa-miR-153_st | −0.322603156 | 1.3 | 0.208263 | 0 |
| hsa-miR-1537_st | −0.178492716 | 1.1 | 0.780523 | 0 |
| hsa-miR-1538_st | −0.254992422 | 1.2 | 0.413787 | 0 |
| hsa-miR-1539_st | −0.052246194 | 1.0 | 0.752834 | 0 |
| hsa-miR-154_st | 0.259776045 | 1.2 | 0.365195 | 0 |
| hsa-miR-154-star_st | 0.498869548 | 1.4 | 0.070034 | 0 |
| hsa-miR-432-star_st | 0.577613365 | 1.5 | 0.042533 | 1 |
| hsa-miR-155-star_st | −0.42954563 | 1.3 | 0.094111 | 0 |
| hsa-miR-298_st | 0.189320666 | 1.1 | 0.269543 | 0 |
| hsa-miR-15a-star_st | 0.211556396 | 1.2 | 0.631205 | 0 |
| hsa-miR-2110_st | 0.185897965 | 1.1 | 0.444748 | 0 |
| hsa-miR-15b-star_st | 0.028843654 | 1.0 | 0.723751 | 0 |
| hsa-miR-1266_st | 0.095158208 | 1.1 | 0.449161 | 0 |
| hsa-miR-16-1-star_st | 0.129889439 | 1.1 | 0.926047 | 0 |
| hsa-miR-16-2-star_st | 0.186499866 | 1.1 | 0.941051 | 0 |
| hsa-miR-490-5p_st | 0.272356707 | 1.2 | 0.299292 | 0 |
| hsa-miR-19a_st | 0.577445133 | 1.5 | 0.585889 | 0 |
| hsa-miR-3192_st | 0.119080009 | 1.1 | 0.231625 | 0 |
| hsa-miR-3155_st | 0.36929577 | 1.3 | 0.016146 | 1 |
| hsa-miR-187-star_st | −0.040562667 | 1.0 | 0.993231 | 0 |
| hsa-miR-16_st | 0.220122823 | 1.2 | 0.916486 | 0 |
| hsa-miR-936_st | 0.110515944 | 1.1 | 0.314959 | 0 |
| hsa-miR-181c-star_st | 0.240336314 | 1.2 | 0.462049 | 0 |
| hsa-miR-379_st | 0.661853147 | 1.6 | 0.00289 | 1 |
| hsa-miR-370_st | 0.006963913 | 1.0 | 0.851394 | 0 |
| hsa-miR-541_st | 0.190010878 | 1.1 | 0.352851 | 0 |
| hsa-miR-518c-star_st | 0.192841849 | 1.1 | 0.270646 | 0 |
| hsa-miR-487b_st | 0.298745781 | 1.2 | 0.842984 | 0 |
| hsa-miR-182-star_st | −0.252195791 | 1.2 | 0.25357 | 0 |
| hsa-miR-3121_st | 0.358597185 | 1.3 | 0.49505 | 0 |
| hsa-miR-3190-5p_st | 0.065396988 | 1.0 | 0.40608 | 0 |
| hsa-miR-1203_st | 0.151413387 | 1.1 | 0.391938 | 0 |
| hsa-miR-185_st | 0.144429706 | 1.1 | 0.961782 | 0 |
| hsa-miR-185-star_st | −0.086787621 | 1.1 | 0.545045 | 0 |
| hsa-miR-4317_st | 0.31451382 | 1.2 | 0.479108 | 0 |
| hsa-miR-186-star_st | 0.016389215 | 1.0 | 0.832989 | 0 |
| hsa-miR-187_st | 0.444649968 | 1.4 | 0.157438 | 0 |
| hsa-miR-4257_st | 0.029746602 | 1.0 | 0.981628 | 0 |
| hsa-miR-188-3p_st | 0.273185757 | 1.2 | 0.305809 | 0 |
| hsa-miR-1184_st | 0.589679414 | 1.5 | 0.05273 | 0 |
| hsa-miR-18a_st | 0.406035907 | 1.3 | 0.120102 | 0 |
| hsa-miR-18a-star_st | 0.298369992 | 1.2 | 0.397917 | 0 |
| hsa-miR-3140_st | 0.542698169 | 1.5 | 0.029979 | 1 |
| hsa-miR-18b-star_st | 0.139194953 | 1.1 | 0.407341 | 0 |
| hsa-miR-190_st | −0.359433873 | 1.3 | 0.174358 | 0 |
| hsa-miR-601_st | 0.08803042 | 1.1 | 0.71448 | 0 |
| hsa-miR-1909_st | −0.218546252 | 1.2 | 0.785095 | 0 |
| hsa-miR-342-5p_st | 0.535361666 | 1.4 | 0.042474 | 1 |
| hsa-miR-575_st | 0.049263204 | 1.0 | 0.412505 | 0 |
| hsa-miR-885-5p_st | 0.222495867 | 1.2 | 0.301399 | 0 |
| hsa-miR-300_st | 0.268796058 | 1.2 | 0.176561 | 0 |
| hsa-miR-1911_st | 0.166743537 | 1.1 | 0.333433 | 0 |
| hsa-miR-1911-star_st | 0.572929009 | 1.5 | 0.051711 | 0 |
| hsa-miR-1912_st | 0.146955006 | 1.1 | 0.317028 | 0 |
| hsa-miR-4266_st | 0.145134038 | 1.1 | 0.042949 | 1 |
| hsa-miR-1914_st | 0.070628747 | 1.1 | 0.59658 | 0 |
| hsa-miR-3190-3p_st | −0.037331733 | 1.0 | 0.78176 | 0 |
| hsa-miR-618_st | 0.494306949 | 1.4 | 0.04247 | 1 |
| hsa-miR-1276_st | 0.00351847 | 1.0 | 0.360422 | 0 |
| hsa-miR-191-star_st | 0.147510694 | 1.1 | 0.520521 | 0 |
| hsa-miR-1258_st | 0.275840254 | 1.2 | 0.04271 | 1 |
| hsa-miR-192-star_st | −0.06376955 | 1.0 | 0.525846 | 0 |
| hsa-miR-193a-3p_st | 0.022503659 | 1.0 | 0.880142 | 0 |
| hsa-miR-193a-5p_st | −0.403890343 | 1.3 | 0.21739 | 0 |
| hsa-miR-3157_st | 0.248581098 | 1.2 | 0.094905 | 0 |
| hsa-miR-193b-star_st | −0.169180628 | 1.1 | 0.710155 | 0 |
| hsa-miR-194_st | −0.20492966 | 1.2 | 0.528825 | 0 |
| hsa-miR-4307_st | 0.562970988 | 1.5 | 0.016437 | 1 |
| hsa-miR-195_st | −0.360440708 | 1.3 | 0.10651 | 0 |
| hsa-miR-610_st | 0.438112863 | 1.4 | 0.03075 | 1 |
| hsa-miR-196a_st | 0.144397279 | 1.1 | 0.986012 | 0 |
| hsa-miR-196a-star_st | 0.060957058 | 1.0 | 0.279451 | 0 |
| hsa-miR-196b_st | −0.316349895 | 1.2 | 0.08753 | 0 |
| hsa-miR-196b-star_st | 0.19969431 | 1.1 | 0.30428 | 0 |
| hsa-let-7f-2-star_st | 0.448215724 | 1.4 | 0.027173 | 1 |
| hsa-miR-1972_st | 0.090137215 | 1.1 | 0.938615 | 0 |
| hsa-miR-3184_st | 0.020283069 | 1.0 | 0.220167 | 0 |
| hsa-miR-523-star_st | 0.161139449 | 1.1 | 0.341426 | 0 |
| hsa-miR-1976_st | 0.442441254 | 1.4 | 0.426464 | 0 |
| hsa-miR-625-star_st | 0.429714114 | 1.3 | 0.001811 | 1 |
| hsa-miR-587_st | 0.220743245 | 1.2 | 0.359828 | 0 |
| hsa-miR-199a-3p_st | 0.585986358 | 1.5 | 0.068287 | 0 |
| hsa-miR-1910_st | 0.139234923 | 1.1 | 0.14963 | 0 |
| hsa-miR-199b-3p_st | 0.352619157 | 1.3 | 0.189896 | 0 |
| hsa-miR-639_st | −0.052286492 | 1.0 | 0.608086 | 0 |
| hsa-miR-1909-star_st | 0.383667591 | 1.3 | 0.006294 | 1 |
| hsa-miR-19a-star_st | −0.272056542 | 1.2 | 0.287445 | 0 |
| hsa-miR-29c-star_st | −0.559030052 | 1.5 | 0.256118 | 0 |
| hsa-miR-940_st | 0.069355857 | 1.0 | 0.287876 | 0 |
| hsa-miR-19b-2-star_st | −0.09964156 | 1.1 | 0.818241 | 0 |
| hsa-miR-4269_st | 0.221680435 | 1.2 | 0.126453 | 0 |
| hsa-miR-200a-star_st | 0.130237044 | 1.1 | 0.359164 | 0 |
| hsa-miR-1182_st | −0.056682666 | 1.0 | 0.597884 | 0 |
| hsa-miR-518f-star_st | 0.382805669 | 1.3 | 0.023402 | 1 |
| hsa-miR-92a-2-star_st | 0.065203117 | 1.0 | 0.319181 | 0 |
| hsa-miR-200c-star_st | −0.07689136 | 1.1 | 0.607763 | 0 |
| hsa-miR-202_st | 0.132661941 | 1.1 | 0.420546 | 0 |
| hsa-miR-202-star_st | −0.416230508 | 1.3 | 0.139646 | 0 |
| hsa-miR-1914-star_st | 0.039930461 | 1.0 | 0.71646 | 0 |
| hsa-miR-204_st | −1.360806389 | 2.6 | 0.235145 | 0 |
| hsa-miR-665_st | 0.152707278 | 1.1 | 0.142516 | 0 |
| hsa-miR-2052_st | −0.250939013 | 1.2 | 0.25587 | 0 |
| hsa-miR-2053_st | −0.29620062 | 1.2 | 0.31199 | 0 |
| hsa-miR-2054_st | −0.427536801 | 1.3 | 0.32845 | 0 |
| hsa-miR-205-star_st | −0.061640286 | 1.0 | 0.973845 | 0 |
| hsa-miR-206_st | 0.454007399 | 1.4 | 0.056038 | 0 |
| hsa-miR-208a_st | −0.084780951 | 1.1 | 0.740568 | 0 |
| hsa-miR-208b_st | 0.085598792 | 1.1 | 0.731521 | 0 |
| hsa-miR-18b_st | 0.350917932 | 1.3 | 0.012977 | 1 |
| hsa-miR-20a-star_st | 0.356726338 | 1.3 | 0.659528 | 0 |
| hsa-miR-3181_st | −0.073987421 | 1.1 | 0.581726 | 0 |
| hsa-miR-615-5p_st | 0.050775889 | 1.0 | 0.117573 | 0 |
| hsa-miR-4259_st | −0.088211305 | 1.1 | 0.739551 | 0 |
| hsa-miR-210_st | −0.198670803 | 1.1 | 0.754396 | 0 |
| hsa-miR-211_st | 0.083674094 | 1.1 | 0.644261 | 0 |
| hsa-miR-887_st | 0.088567536 | 1.1 | 0.207835 | 0 |
| hsa-miR-2113_st | 0.682135969 | 1.6 | 0.12567 | 0 |
| hsa-miR-2114_st | 0.173258011 | 1.1 | 0.224163 | 0 |
| hsa-miR-943_st | 0.017562171 | 1.0 | 0.864482 | 0 |
| hsa-miR-2115_st | −0.09669143 | 1.1 | 0.60714 | 0 |
| hsa-miR-2115-star_st | −0.266414089 | 1.2 | 0.199105 | 0 |
| hsa-miR-183-star_st | 0.240227093 | 1.2 | 0.399326 | 0 |
| hsa-miR-2116-star_st | 0.341008264 | 1.3 | 0.131291 | 0 |
| hsa-miR-920_st | 0.024508378 | 1.0 | 0.544312 | 0 |
| hsa-miR-212_st | 0.324357181 | 1.3 | 0.070228 | 0 |
| hsa-miR-214_st | 0.278133685 | 1.2 | 0.137009 | 0 |
| hsa-miR-1825_st | 0.364615919 | 1.3 | 0.069404 | 0 |
| hsa-miR-215_st | 0.703710939 | 1.6 | 0.168019 | 0 |
| hsa-miR-216a_st | 0.028005855 | 1.0 | 0.947679 | 0 |
| hsa-miR-195-star_st | 0.535808084 | 1.4 | 0.082091 | 0 |
| hsa-miR-217_st | −0.248213761 | 1.2 | 0.261048 | 0 |
| hsa-miR-23a-star_st | 0.088641418 | 1.1 | 0.587624 | 0 |
| hsa-miR-25-star_st | −0.009084933 | 1.0 | 0.486094 | 0 |
| hsa-miR-218-2-star_st | 0.118423806 | 1.1 | 0.2265 | 0 |
| hsa-miR-1273c_st | 0.249749016 | 1.2 | 0.584105 | 0 |
| hsa-miR-219-2-3p_st | 0.040706344 | 1.0 | 0.529187 | 0 |
| hsa-miR-637_st | −0.133965798 | 1.1 | 0.829827 | 0 |
| hsa-miR-21-star_st | 0.628304942 | 1.5 | 0.053729 | 0 |
| hsa-miR-22_st | −0.481594244 | 1.4 | 0.133164 | 0 |
| hsa-miR-220a_st | −0.089116741 | 1.1 | 0.722455 | 0 |
| hsa-miR-220b_st | −0.084712989 | 1.1 | 0.929793 | 0 |
| hsa-miR-219-1-3p_st | 0.240275924 | 1.2 | 0.057287 | 0 |
| hsa-miR-4316_st | 0.037435645 | 1.0 | 0.647248 | 0 |
| hsa-miR-877_st | −0.074134772 | 1.1 | 0.914585 | 0 |
| hsa-miR-636_st | 0.045553357 | 1.0 | 0.541566 | 0 |
| hsa-miR-3177_st | −0.12101774 | 1.1 | 0.98502 | 0 |
| hsa-miR-1249_st | 0.269924164 | 1.2 | 0.046071 | 1 |
| hsa-miR-223-star_st | 0.392485906 | 1.3 | 0.145991 | 0 |
| hsa-miR-1224-5p_st | −0.041667436 | 1.0 | 0.664727 | 0 |
| hsa-miR-564_st | −0.001983593 | 1.0 | 0.857694 | 0 |
| hsa-miR-2276_st | 0.011875718 | 1.0 | 0.863729 | 0 |
| hsa-miR-2277_st | −0.058464211 | 1.0 | 0.784097 | 0 |
| hsa-miR-2278_st | 0.021208058 | 1.0 | 0.623441 | 0 |
| hsa-miR-22-star_st | −0.475466187 | 1.4 | 0.096217 | 0 |
| hsa-miR-2355_st | 0.197659827 | 1.1 | 0.185229 | 0 |
| hsa-miR-769-3p_st | 0.012423408 | 1.0 | 0.11924 | 0 |
| hsa-miR-1913_st | 0.177330835 | 1.1 | 0.111568 | 0 |
| hsa-miR-1237_st | 0.2642963 | 1.2 | 0.102964 | 0 |
| hsa-miR-1275_st | 0.04839678 | 1.0 | 0.768159 | 0 |
| hsa-miR-30a_st | −0.584746325 | 1.5 | 0.209717 | 0 |
| hsa-miR-20b-star_st | 0.256800895 | 1.2 | 0.312316 | 0 |
| hsa-miR-24-2-star_st | 0.861033828 | 1.8 | 0.061995 | 0 |
| hsa-miR-3173_st | 0.001710887 | 1.0 | 0.540528 | 0 |
| hsa-miR-3148_st | 0.24995585 | 1.2 | 0.289613 | 0 |
| hsa-miR-4271_st | −0.11045805 | 1.1 | 0.368593 | 0 |
| hsa-miR-659_st | −0.030069052 | 1.0 | 0.592931 | 0 |
| hsa-miR-26a-2-star_st | −0.417469575 | 1.3 | 0.121586 | 0 |
| hsa-miR-1228_st | 0.038453849 | 1.0 | 0.131694 | 0 |
| hsa-miR-26b-star_st | −0.073414743 | 1.1 | 0.628726 | 0 |
| hsa-miR-3174_st | 0.264770477 | 1.2 | 0.663021 | 0 |
| hsa-miR-27a-star_st | 0.51646283 | 1.4 | 0.093285 | 0 |
| hsa-miR-1285_st | 0.18888405 | 1.1 | 0.476498 | 0 |
| hsa-miR-483-5p_st | 0.072187585 | 1.1 | 0.617728 | 0 |
| hsa-miR-1306_st | 0.094554231 | 1.1 | 0.479575 | 0 |
| hsa-miR-431_st | 0.195065481 | 1.1 | 0.024129 | 1 |
| hsa-miR-765_st | 0.063301125 | 1.0 | 0.236606 | 0 |
| hsa-miR-2909_st | −0.234768551 | 1.2 | 0.215933 | 0 |
| hsa-miR-296-3p_st | −0.111423297 | 1.1 | 0.803913 | 0 |
| hsa-miR-296-5p_st | 0.171546983 | 1.1 | 0.220193 | 0 |
| hsa-miR-297_st | 0.383566532 | 1.3 | 0.31691 | 0 |
| hsa-miR-323b-5p_st | 0.242872311 | 1.2 | 0.253737 | 0 |
| hsa-miR-299-3p_st | 0.305249178 | 1.2 | 0.139037 | 0 |
| hsa-miR-299-5p_st | −0.247295581 | 1.2 | 0.402571 | 0 |
| hsa-miR-487a_st | 0.081306244 | 1.1 | 0.532351 | 0 |
| hsa-miR-29a-star_st | −0.022166912 | 1.0 | 0.906334 | 0 |
| hsa-miR-625_st | 0.62931785 | 1.5 | 0.000114 | 1 |
| hsa-miR-611_st | 0.073601097 | 1.1 | 0.148153 | 0 |
| hsa-miR-3186-3p_st | 0.114238135 | 1.1 | 0.108442 | 0 |
| hsa-miR-1294_st | 0.221203458 | 1.2 | 0.026848 | 1 |
| hsa-miR-3147_st | −0.17353218 | 1.1 | 0.817427 | 0 |
| hsa-miR-4285_st | −0.23358084 | 1.2 | 0.984675 | 0 |
| hsa-miR-125a-3p_st | 0.130241195 | 1.1 | 0.367499 | 0 |
| hsa-miR-301b_st | −0.120914885 | 1.1 | 0.510285 | 0 |
| hsa-miR-302a_st | −0.119136838 | 1.1 | 0.708886 | 0 |
| hsa-miR-518e-star_st | 0.099173481 | 1.1 | 0.089954 | 0 |
| hsa-miR-302b_st | −0.50018713 | 1.4 | 0.213546 | 0 |
| hsa-miR-302b-star_st | −0.258553005 | 1.2 | 0.131689 | 0 |
| hsa-miR-302c_st | 0.139737716 | 1.1 | 0.406483 | 0 |
| hsa-miR-302c-star_st | −0.269138984 | 1.2 | 0.473404 | 0 |
| hsa-miR-302d_st | −0.134989687 | 1.1 | 0.566297 | 0 |
| hsa-miR-302d-star_st | −0.186876197 | 1.1 | 0.20843 | 0 |
| hsa-miR-302e_st | 0.333448168 | 1.3 | 0.48786 | 0 |
| hsa-miR-302f_st | −0.143545195 | 1.1 | 0.183425 | 0 |
| hsa-miR-3065-3p_st | 0.563470303 | 1.5 | 0.217531 | 0 |
| hsa-miR-3130-5p_st | 0.175832411 | 1.1 | 0.928059 | 0 |
| hsa-miR-3074_st | 0.203009563 | 1.2 | 0.670824 | 0 |
| hsa-miR-1226-star_st | −0.121618421 | 1.1 | 0.949475 | 0 |
| hsa-miR-3180-5p_st | 0.003044053 | 1.0 | 0.857124 | 0 |
| hsa-miR-1229_st | 0.108792883 | 1.1 | 0.631479 | 0 |
| hsa-miR-1272_st | 0.182318542 | 1.1 | 0.070518 | 0 |
| hsa-miR-30c_st | −0.792287033 | 1.7 | 0.075058 | 0 |
| hsa-miR-675_st | −0.107445216 | 1.1 | 0.613588 | 0 |
| hsa-miR-30c-2-star_st | 0.129189904 | 1.1 | 0.275299 | 0 |
| hsa-miR-1827_st | −0.02998404 | 1.0 | 0.346014 | 0 |
| hsa-miR-30d-star_st | −0.079096943 | 1.1 | 0.825503 | 0 |
| hsa-miR-30e_st | −0.269656082 | 1.2 | 0.233162 | 0 |
| hsa-miR-1915-star_st | 0.24386969 | 1.2 | 0.124104 | 0 |
| hsa-miR-1322_st | 0.122735752 | 1.1 | 0.029139 | 1 |
| hsa-miR-3115_st | −0.079423382 | 1.1 | 0.975094 | 0 |
| hsa-miR-3116_st | 0.419493902 | 1.3 | 0.343724 | 0 |
| hsa-miR-3117_st | −0.026244077 | 1.0 | 0.837162 | 0 |
| hsa-miR-3118_st | −0.27832119 | 1.2 | 0.222457 | 0 |
| hsa-miR-519b-5p_st | 0.01482909 | 1.0 | 0.194415 | 0 |
| hsa-miR-3120_st | 0.304269725 | 1.2 | 0.37177 | 0 |
| hsa-miR-520d-3p_st | 0.160049132 | 1.1 | 0.347169 | 0 |
| hsa-miR-326_st | 0.163605958 | 1.1 | 0.344952 | 0 |
| hsa-miR-3123_st | −0.091853526 | 1.1 | 0.605872 | 0 |
| hsa-miR-3124_st | −0.155841051 | 1.1 | 0.673441 | 0 |
| hsa-miR-3125_st | 0.25034966 | 1.2 | 0.194418 | 0 |
| hsa-miR-3126-3p_st | 0.003831294 | 1.0 | 0.928335 | 0 |
| hsa-miR-1296_st | 0.263315112 | 1.2 | 0.055726 | 0 |
| hsa-miR-3127_st | −0.028001945 | 1.0 | 0.941335 | 0 |
| hsa-miR-3128_st | −0.074303791 | 1.1 | 0.529974 | 0 |
| hsa-miR-3129_st | 0.074971515 | 1.1 | 0.591394 | 0 |
| hsa-miR-3130-3p_st | 0.101394559 | 1.1 | 0.36222 | 0 |
| hsa-miR-105-star_st | 0.204390446 | 1.2 | 0.066377 | 0 |
| hsa-miR-3131_st | −0.019945016 | 1.0 | 0.470769 | 0 |
| hsa-miR-4255_st | 0.181356724 | 1.1 | 0.013821 | 1 |
| hsa-miR-3133_st | −0.232020849 | 1.2 | 0.317196 | 0 |
| hsa-miR-3134_st | 0.231473678 | 1.2 | 0.226633 | 0 |
| hsa-miR-3135_st | 0.171198221 | 1.1 | 0.641851 | 0 |
| hsa-miR-3136_st | −0.124747728 | 1.1 | 0.721364 | 0 |
| hsa-miR-19b-1-star_st | −0.117639424 | 1.1 | 0.934124 | 0 |
| hsa-miR-1247_st | −0.143941086 | 1.1 | 0.565652 | 0 |
| hsa-miR-3139_st | 0.046745612 | 1.0 | 0.286044 | 0 |
| hsa-miR-596_st | 0.098567312 | 1.1 | 0.077547 | 0 |
| hsa-miR-3141_st | −0.324538175 | 1.3 | 0.370717 | 0 |
| hsa-miR-3142_st | −0.334752295 | 1.3 | 0.293943 | 0 |
| hsa-miR-1302_st | −0.202628829 | 1.2 | 0.245605 | 0 |
| hsa-miR-3144-3p_st | 0.468244668 | 1.4 | 0.257366 | 0 |
| hsa-miR-3144-5p_st | 0.552061596 | 1.5 | 0.15142 | 0 |
| hsa-miR-3145_st | −0.052818993 | 1.0 | 0.876659 | 0 |
| hsa-miR-3146_st | 0.114071261 | 1.1 | 0.725827 | 0 |
| hsa-miR-346_st | 0.082782051 | 1.1 | 0.711318 | 0 |
| hsa-miR-4256_st | 0.055587343 | 1.0 | 0.190026 | 0 |
| hsa-miR-3149_st | 0.264424446 | 1.2 | 0.301062 | 0 |
| hsa-miR-3150_st | −0.057615289 | 1.0 | 0.377404 | 0 |
| hsa-miR-3151_st | −0.13683372 | 1.1 | 0.735127 | 0 |
| hsa-miR-1470_st | 0.06309613 | 1.0 | 0.0732 | 0 |
| hsa-miR-3153_st | 0.158659896 | 1.1 | 0.31969 | 0 |
| hsa-miR-514_st | −0.255112571 | 1.2 | 0.024471 | 1 |
| hsa-miR-3175_st | −0.090171011 | 1.1 | 0.025631 | 1 |
| hsa-miR-3156_st | 0.01042151 | 1.0 | 0.663664 | 0 |
| hsa-miR-504_st | −0.354647647 | 1.3 | 0.248216 | 0 |
| hsa-miR-3158_st | 0.242012898 | 1.2 | 0.387274 | 0 |
| hsa-miR-3159_st | 0.130087668 | 1.1 | 0.220911 | 0 |
| hsa-miR-3160_st | 0.361948767 | 1.3 | 0.25058 | 0 |
| hsa-miR-3161_st | 0.33659093 | 1.3 | 0.859574 | 0 |
| hsa-miR-3162_st | −0.085678651 | 1.1 | 0.524787 | 0 |
| hsa-miR-1234_st | −0.091927645 | 1.1 | 0.030069 | 1 |
| hsa-miR-3164_st | 0.346374988 | 1.3 | 0.266081 | 0 |
| hsa-miR-516b-star_st | −0.203440083 | 1.2 | 0.215677 | 0 |
| hsa-miR-3166_st | −0.181213397 | 1.1 | 0.660455 | 0 |
| hsa-miR-4327_st | −0.233728282 | 1.2 | 0.41463 | 0 |
| hsa-miR-3168_st | −0.216039337 | 1.2 | 0.57451 | 0 |
| hsa-miR-3169_st | 0.051137454 | 1.0 | 0.552598 | 0 |
| hsa-miR-3170_st | 0.320988357 | 1.2 | 0.148907 | 0 |
| hsa-miR-3171_st | −0.242069253 | 1.2 | 0.136424 | 0 |
| hsa-miR-3172_st | −0.515194022 | 1.4 | 0.070227 | 0 |
| hsa-miR-374b-star_st | −0.135881605 | 1.1 | 0.166965 | 0 |
| hsa-miR-34c-5p_st | −0.137648123 | 1.1 | 0.232896 | 0 |
| hsa-miR-758_st | −0.42411508 | 1.3 | 0.018479 | 1 |
| hsa-miR-3176_st | −0.394622078 | 1.3 | 0.575357 | 0 |
| hsa-miR-606_st | −0.238257927 | 1.2 | 0.14124 | 0 |
| hsa-miR-3178_st | −0.313925385 | 1.2 | 0.268607 | 0 |
| hsa-miR-3179_st | 0.026344761 | 1.0 | 0.8203 | 0 |
| hsa-miR-3180-3p_st | −0.336927668 | 1.3 | 0.412272 | 0 |
| hsa-miR-597_st | −0.226188253 | 1.2 | 0.14718 | 0 |
| hsa-miR-148b-star_st | −0.196406099 | 1.1 | 0.298728 | 0 |
| hsa-miR-3182_st | 0.078928423 | 1.1 | 0.800725 | 0 |
| hsa-miR-3183_st | 0.119947781 | 1.1 | 0.721543 | 0 |
| hsa-miR-144-star_st | −0.306543697 | 1.2 | 0.036562 | 1 |
| hsa-miR-3185_st | −0.398312207 | 1.3 | 0.214967 | 0 |
| hsa-miR-4270_st | −0.311916829 | 1.2 | 0.187261 | 0 |
| hsa-miR-3186-5p_st | 0.268683048 | 1.2 | 0.485646 | 0 |
| hsa-miR-3187_st | −0.195651659 | 1.1 | 0.630086 | 0 |
| hsa-miR-3188_st | −0.233796974 | 1.2 | 0.37223 | 0 |
| hsa-miR-3189_st | 0.013209335 | 1.0 | 0.303601 | 0 |
| hsa-miR-548b-3p_st | −0.394103938 | 1.3 | 0.16976 | 0 |
| hsa-miR-99a-star_st | −0.14210008 | 1.1 | 0.267534 | 0 |
| hsa-miR-376a-star_st | −0.438856306 | 1.4 | 0.026751 | 1 |
| hsa-miR-216b_st | −0.254423156 | 1.2 | 0.551732 | 0 |
| hsa-miR-3193_st | 0.129391264 | 1.1 | 0.743454 | 0 |
| hsa-miR-548v_st | −0.415816203 | 1.3 | 0.008072 | 1 |
| hsa-miR-26a-1-star_st | 0.061401517 | 1.0 | 0.689128 | 0 |
| hsa-miR-1291_st | −0.16112209 | 1.1 | 0.0205 | 1 |
| hsa-miR-3197_st | −0.120456811 | 1.1 | 0.361294 | 0 |
| hsa-miR-629_st | −0.037895518 | 1.0 | 0.453804 | 0 |
| hsa-miR-3199_st | 0.135592462 | 1.1 | 0.499345 | 0 |
| hsa-miR-190b_st | −0.480972754 | 1.4 | 0.001896 | 1 |
| hsa-miR-32_st | −0.419442578 | 1.3 | 0.164795 | 0 |
| hsa-miR-3200_st | −0.126930699 | 1.1 | 0.493669 | 0 |
| hsa-miR-3201_st | −0.036710166 | 1.0 | 0.821673 | 0 |
| hsa-miR-3202_st | 0.402999655 | 1.3 | 0.101864 | 0 |
| hsa-miR-320a_st | −0.251451643 | 1.2 | 0.558959 | 0 |
| hsa-miR-4289_st | −0.270829537 | 1.2 | 0.597481 | 0 |
| hsa-miR-655_st | −0.368556761 | 1.3 | 0.158592 | 0 |
| hsa-miR-320b_st | −0.35253325 | 1.3 | 0.186899 | 0 |
| hsa-miR-219-5p_st | −0.328121945 | 1.3 | 0.051686 | 0 |
| hsa-miR-323-3p_st | 0.125301285 | 1.1 | 0.673919 | 0 |
| hsa-miR-323-5p_st | −0.052004644 | 1.0 | 0.975905 | 0 |
| hsa-miR-323b-3p_st | −0.013270906 | 1.0 | 0.333781 | 0 |
| hsa-miR-103-as_st | −0.187222262 | 1.1 | 0.013297 | 1 |
| hsa-miR-424-star_st | −0.228529563 | 1.2 | 0.105266 | 0 |
| hsa-miR-33a-star_st | 0.025569587 | 1.0 | 0.569404 | 0 |
| hsa-miR-325_st | 0.129207092 | 1.1 | 0.591898 | 0 |
| hsa-miR-590-3p_st | −0.33515707 | 1.3 | 0.13373 | 0 |
| hsa-miR-328_st | −0.653137058 | 1.6 | 0.202436 | 0 |
| hsa-miR-329_st | 0.244769264 | 1.2 | 0.259484 | 0 |
| hsa-miR-32-star_st | 0.299083019 | 1.2 | 0.231699 | 0 |
| hsa-miR-330-3p_st | 0.383022441 | 1.3 | 0.090106 | 0 |
| hsa-miR-330-5p_st | 0.006143569 | 1.0 | 0.445408 | 0 |
| hsa-miR-146a-star_st | −0.257439252 | 1.2 | 0.097765 | 0 |
| hsa-miR-331-5p_st | 0.066384289 | 1.0 | 0.942027 | 0 |
| hsa-miR-3167_st | −0.259626583 | 1.2 | 0.287251 | 0 |
| hsa-miR-335-star_st | 0.358874478 | 1.3 | 0.792819 | 0 |
| hsa-miR-337-3p_st | 0.479395538 | 1.4 | 0.292348 | 0 |
| hsa-miR-337-5p_st | 0.137743692 | 1.1 | 0.159703 | 0 |
| hsa-miR-421_st | 0.119392636 | 1.1 | 0.7771 | 0 |
| hsa-miR-338-5p_st | 0.038763558 | 1.0 | 0.491261 | 0 |
| hsa-miR-339-3p_st | −0.042717967 | 1.0 | 0.463727 | 0 |
| hsa-miR-548a-5p_st | −0.429059365 | 1.3 | 0.014966 | 1 |
| hsa-miR-33a_st | −0.313173638 | 1.2 | 0.245048 | 0 |
| hsa-miR-590-5p_st | −0.211078204 | 1.2 | 0.250467 | 0 |
| hsa-miR-33b_st | −0.287905206 | 1.2 | 0.33353 | 0 |
| hsa-miR-616-star_st | −0.392399674 | 1.3 | 0.030131 | 1 |
| hsa-miR-569_st | −0.315730178 | 1.2 | 0.156427 | 0 |
| hsa-miR-340-star_st | −0.173004694 | 1.1 | 0.329519 | 0 |
| hsa-miR-342-3p_st | 0.872504154 | 1.8 | 0.156627 | 0 |
| hsa-miR-135b-star_st | −0.264994046 | 1.2 | 0.014113 | 1 |
| hsa-miR-345_st | −0.716548174 | 1.6 | 0.161559 | 0 |
| hsa-miR-1288_st | 0.083971989 | 1.1 | 0.922635 | 0 |
| hsa-miR-135a_st | −0.426929078 | 1.3 | 0.000433 | 1 |
| hsa-miR-34a-star_st | 0.434256293 | 1.4 | 0.881006 | 0 |
| hsa-miR-34b_st | 0.510326166 | 1.4 | 0.099675 | 0 |
| hsa-miR-4288_st | −0.202327966 | 1.2 | 0.044094 | 1 |
| hsa-miR-222-star_st | 0.903411912 | 1.9 | 0.024026 | 1 |
| hsa-miR-933_st | −0.261696727 | 1.2 | 0.269561 | 0 |
| hsa-miR-361-3p_st | −0.194631233 | 1.1 | 0.971136 | 0 |
| hsa-miR-224-star_st | −0.141823241 | 1.1 | 0.837286 | 0 |
| hsa-miR-362-3p_st | 0.601119039 | 1.5 | 0.515 | 0 |
| hsa-miR-362-5p_st | 0.124069134 | 1.1 | 0.770287 | 0 |
| hsa-miR-363_st | 0.763458508 | 1.7 | 0.126319 | 0 |
| hsa-miR-363-star_st | 0.228016924 | 1.2 | 0.096887 | 0 |
| hsa-miR-1979_st | −0.584991801 | 1.5 | 0.019293 | 1 |
| hsa-miR-365-star_st | −0.202570494 | 1.2 | 0.738511 | 0 |
| hsa-miR-548x_st | −0.327986435 | 1.3 | 0.07609 | 0 |
| hsa-miR-367-star_st | −0.025933456 | 1.0 | 0.94268 | 0 |
| hsa-miR-369-3p_st | −0.112820683 | 1.1 | 0.52116 | 0 |
| hsa-miR-369-5p_st | 0.003359916 | 1.0 | 0.927553 | 0 |
| hsa-miR-548l_st | 0.048681172 | 1.0 | 0.756705 | 0 |
| hsa-miR-371-3p_st | 0.218615609 | 1.2 | 0.245427 | 0 |
| hsa-miR-371-5p_st | −0.238001731 | 1.2 | 0.725143 | 0 |
| hsa-miR-372_st | 0.452444944 | 1.4 | 0.068495 | 0 |
| hsa-miR-373_st | 0.079260487 | 1.1 | 0.390916 | 0 |
| hsa-miR-373-star_st | −0.203624504 | 1.2 | 0.979082 | 0 |
| hsa-miR-374a_st | 0.114727769 | 1.1 | 0.538862 | 0 |
| hsa-miR-374a-star_st | −0.303801191 | 1.2 | 0.383062 | 0 |
| hsa-miR-500_st | −0.269811853 | 1.2 | 0.20076 | 0 |
| hsa-miR-320c_st | −0.428438761 | 1.3 | 0.227782 | 0 |
| hsa-miR-875-3p_st | −0.067111025 | 1.0 | 0.410409 | 0 |
| hsa-miR-376a_st | 0.451197028 | 1.4 | 0.110634 | 0 |
| hsa-miR-29b-1-star_st | 0.568250198 | 1.5 | 0.03902 | 1 |
| hsa-miR-376b_st | −0.106094916 | 1.1 | 0.823776 | 0 |
| hsa-miR-376c_st | 0.521808818 | 1.4 | 0.344991 | 0 |
| hsa-miR-377_st | 0.071463265 | 1.1 | 0.61359 | 0 |
| hsa-miR-377-star_st | 0.057005002 | 1.0 | 0.824696 | 0 |
| hsa-miR-378_st | 0.131939567 | 1.1 | 0.902688 | 0 |
| hsa-miR-378b_st | −0.083826382 | 1.1 | 0.981489 | 0 |
| hsa-miR-378c_st | 0.27251436 | 1.2 | 0.059742 | 0 |
| hsa-miR-378-star_st | −0.052134955 | 1.0 | 0.768495 | 0 |
| hsa-miR-662_st | −0.05383721 | 1.0 | 0.0675 | 0 |
| hsa-miR-379-star_st | −0.06747124 | 1.0 | 0.558628 | 0 |
| hsa-miR-380_st | −0.199051186 | 1.1 | 0.358806 | 0 |
| hsa-miR-380-star_st | 0.289924324 | 1.2 | 0.05639 | 0 |
| hsa-miR-1274b_st | −0.029966248 | 1.0 | 0.035583 | 1 |
| hsa-miR-382_st | 0.111919473 | 1.1 | 0.656666 | 0 |
| hsa-miR-383_st | 0.293267945 | 1.2 | 0.229382 | 0 |
| hsa-miR-384_st | −0.392836875 | 1.3 | 0.300101 | 0 |
| hsa-miR-126-star_st | −0.399345752 | 1.3 | 0.034858 | 1 |
| hsa-miR-409-5p_st | −0.12414789 | 1.1 | 0.874792 | 0 |
| hsa-miR-410_st | 0.006697404 | 1.0 | 0.469478 | 0 |
| hsa-miR-411_st | 0.271150668 | 1.2 | 0.246715 | 0 |
| hsa-miR-411-star_st | 0.309570329 | 1.2 | 0.261571 | 0 |
| hsa-miR-412_st | 0.071442552 | 1.1 | 0.540408 | 0 |
| hsa-miR-3143_st | −0.316632811 | 1.2 | 0.158183 | 0 |
| hsa-miR-422a_st | 0.105923567 | 1.1 | 0.88658 | 0 |
| hsa-miR-628-5p_st | −0.453495757 | 1.4 | 0.029429 | 1 |
| hsa-miR-423-5p_st | −0.096535656 | 1.1 | 0.905582 | 0 |
| hsa-miR-27b-star_st | 0.445837838 | 1.4 | 0.219383 | 0 |
| hsa-miR-499-5p_st | −0.374969431 | 1.3 | 0.012163 | 1 |
| hsa-miR-425_st | 0.214264038 | 1.2 | 0.73765 | 0 |
| hsa-miR-193b_st | −0.328445081 | 1.3 | 0.985986 | 0 |
| hsa-miR-4252_st | 0.041226352 | 1.0 | 0.625325 | 0 |
| hsa-miR-4253_st | −0.18368008 | 1.1 | 0.810606 | 0 |
| hsa-miR-4254_st | 0.287856229 | 1.2 | 0.334793 | 0 |
| hsa-miR-324-3p_st | −0.547703445 | 1.5 | 0.015682 | 1 |
| hsa-miR-4329_st | −0.409865962 | 1.3 | 0.171829 | 0 |
| hsa-miR-1251_st | −0.299625111 | 1.2 | 0.219551 | 0 |
| hsa-miR-4258_st | 0.107164085 | 1.1 | 0.552863 | 0 |
| hsa-miR-1274a_st | 0.416373316 | 1.3 | 0.212083 | 0 |
| hsa-miR-425-star_st | 0.285997573 | 1.2 | 0.231456 | 0 |
| hsa-miR-4260_st | −0.114314779 | 1.1 | 0.916543 | 0 |
| hsa-miR-4261_st | 0.217255454 | 1.2 | 0.584209 | 0 |
| hsa-miR-4262_st | 0.027979812 | 1.0 | 0.908204 | 0 |
| hsa-miR-4263_st | 0.185457336 | 1.1 | 0.186157 | 0 |
| hsa-miR-4264_st | −0.082514104 | 1.1 | 0.796352 | 0 |
| hsa-miR-1280_st | −0.499265528 | 1.4 | 0.028172 | 1 |
| hsa-miR-132-star_st | 0.195738177 | 1.1 | 0.537453 | 0 |
| hsa-miR-4267_st | −0.39549593 | 1.3 | 0.705016 | 0 |
| hsa-miR-4268_st | −0.24158889 | 1.2 | 0.297297 | 0 |
| hsa-miR-133a_st | −0.799885056 | 1.7 | 0.247447 | 0 |
| hsa-miR-888-star_st | 0.002338696 | 1.0 | 0.795839 | 0 |
| hsa-miR-1260b_st | −0.499880282 | 1.4 | 0.011008 | 1 |
| hsa-miR-4272_st | −0.274324827 | 1.2 | 0.290413 | 0 |
| hsa-miR-4273_st | 0.160997101 | 1.1 | 0.884204 | 0 |
| hsa-miR-4274_st | −0.00677725 | 1.0 | 0.366125 | 0 |
| hsa-miR-4275_st | −0.378201099 | 1.3 | 0.076063 | 0 |
| hsa-miR-4276_st | −0.1758286 | 1.1 | 0.987038 | 0 |
| hsa-miR-4277_st | 0.103006907 | 1.1 | 0.547518 | 0 |
| hsa-miR-4278_st | −0.201991483 | 1.2 | 0.840443 | 0 |
| hsa-miR-4279_st | 0.203748079 | 1.2 | 0.386984 | 0 |
| hsa-miR-4280_st | 0.480746858 | 1.4 | 0.180613 | 0 |
| hsa-miR-4281_st | −0.524773798 | 1.4 | 0.059731 | 0 |
| hsa-miR-4282_st | −0.228979747 | 1.2 | 0.255901 | 0 |
| hsa-miR-4283_st | −0.049374227 | 1.0 | 0.729987 | 0 |
| hsa-miR-4284_st | −0.206951741 | 1.2 | 0.131033 | 0 |
| hsa-miR-23b-star_st | −0.398573391 | 1.3 | 0.156942 | 0 |
| hsa-miR-574-3p_st | −0.532914051 | 1.4 | 0.143724 | 0 |
| hsa-miR-4287_st | 0.324642699 | 1.3 | 0.340234 | 0 |
| hsa-miR-320e_st | −0.40465884 | 1.3 | 0.378533 | 0 |
| hsa-miR-339-5p_st | 0.018246264 | 1.0 | 0.58179 | 0 |
| hsa-miR-505_st | −0.230353564 | 1.2 | 0.264633 | 0 |
| hsa-miR-4290_st | 0.536720356 | 1.5 | 0.117718 | 0 |
| hsa-miR-200b-star_st | −0.5678105 | 1.5 | 0.293779 | 0 |
| hsa-miR-4292_st | −0.0684024 | 1.0 | 0.379099 | 0 |
| hsa-miR-4293_st | 0.433694082 | 1.4 | 0.22694 | 0 |
| hsa-miR-1246_st | −0.633282983 | 1.6 | 0.072857 | 0 |
| hsa-miR-4295_st | 0.134779474 | 1.1 | 0.858855 | 0 |
| hsa-miR-4296_st | 0.125746169 | 1.1 | 0.280132 | 0 |
| hsa-miR-4297_st | 0.546046016 | 1.5 | 0.548689 | 0 |
| hsa-miR-4298_st | −0.126378752 | 1.1 | 0.408526 | 0 |
| hsa-miR-218_st | 0.433498689 | 1.4 | 0.282421 | 0 |
| hsa-miR-4286_st | −0.150485561 | 1.1 | 0.011504 | 1 |
| hsa-miR-4301_st | −0.395404911 | 1.3 | 0.232709 | 0 |
| hsa-miR-4302_st | −0.149952641 | 1.1 | 0.96444 | 0 |
| hsa-miR-365_st | −0.796256029 | 1.7 | 0.028729 | 1 |
| hsa-miR-4304_st | 0.340604197 | 1.3 | 0.210005 | 0 |
| hsa-miR-4305_st | −0.136916788 | 1.1 | 0.561748 | 0 |
| hsa-miR-4306_st | 0.298020807 | 1.2 | 0.230751 | 0 |
| hsa-miR-26a_st | −0.38244862 | 1.3 | 0.225316 | 0 |
| hsa-miR-4308_st | −0.143032922 | 1.1 | 0.60723 | 0 |
| hsa-miR-4309_st | −0.098478831 | 1.1 | 0.509835 | 0 |
| hsa-miR-186_st | −0.149823159 | 1.1 | 0.622279 | 0 |
| hsa-miR-4310_st | 0.229518119 | 1.2 | 0.170438 | 0 |
| hsa-miR-4311_st | 0.289005441 | 1.2 | 0.123099 | 0 |
| hsa-miR-4312_st | 0.256785738 | 1.2 | 0.627573 | 0 |
| hsa-miR-4313_st | −0.324229329 | 1.3 | 0.231956 | 0 |
| hsa-miR-4314_st | 0.396062469 | 1.3 | 0.187354 | 0 |
| hsa-miR-4315_st | 0.505194646 | 1.4 | 0.101405 | 0 |
| hsa-miR-149_st | −0.157854206 | 1.1 | 0.434131 | 0 |
| hsa-miR-106b-star_st | −0.398408826 | 1.3 | 0.704568 | 0 |
| hsa-miR-4318_st | 0.264399722 | 1.2 | 0.272967 | 0 |
| hsa-miR-4319_st | 0.392306268 | 1.3 | 0.30148 | 0 |
| hsa-miR-29b-2-star_st | −0.202934828 | 1.2 | 0.409929 | 0 |
| hsa-miR-432_st | −0.197757106 | 1.1 | 0.700814 | 0 |
| hsa-miR-4320_st | 0.055104544 | 1.0 | 0.855596 | 0 |
| hsa-miR-522_st | −0.243105072 | 1.2 | 0.088311 | 0 |
| hsa-miR-4322_st | −0.254153034 | 1.2 | 0.635321 | 0 |
| hsa-miR-4323_st | 0.240735608 | 1.2 | 0.090052 | 0 |
| hsa-miR-93-star_st | −0.443254552 | 1.4 | 0.023149 | 1 |
| hsa-miR-4325_st | 0.029003291 | 1.0 | 0.850711 | 0 |
| hsa-miR-4326_st | 0.019063849 | 1.0 | 0.717733 | 0 |
| hsa-miR-874_st | −0.016194793 | 1.0 | 0.450244 | 0 |
| hsa-miR-4328_st | 0.039040676 | 1.0 | 0.589432 | 0 |
| hsa-let-7e-star_st | −0.391583213 | 1.3 | 0.363255 | 0 |
| hsa-miR-720_st | −0.522355883 | 1.4 | 0.041567 | 1 |
| hsa-miR-433_st | 0.186443419 | 1.1 | 0.404195 | 0 |
| hsa-miR-4330_st | 0.649618116 | 1.6 | 0.304572 | 0 |
| hsa-miR-448_st | −0.344394475 | 1.3 | 0.420657 | 0 |
| hsa-miR-449a_st | 0.111629787 | 1.1 | 0.275298 | 0 |
| hsa-miR-449b_st | 0.261733339 | 1.2 | 0.298153 | 0 |
| hsa-miR-1975_st | −0.543566756 | 1.5 | 0.03636 | 1 |
| hsa-miR-449c_st | −0.350685049 | 1.3 | 0.826556 | 0 |
| hsa-miR-449c-star_st | 0.198234894 | 1.1 | 0.496224 | 0 |
| hsa-miR-1_st | −0.658541042 | 1.6 | 0.379555 | 0 |
| hsa-miR-450b-3p_st | −0.26865447 | 1.2 | 0.269257 | 0 |
| hsa-miR-450b-5p_st | −0.219833545 | 1.2 | 0.304435 | 0 |
| hsa-miR-451_st | 0.119434024 | 1.1 | 0.851313 | 0 |
| hsa-miR-452_st | 0.481331997 | 1.4 | 0.133942 | 0 |
| hsa-miR-452-star_st | −0.160217591 | 1.1 | 0.184628 | 0 |
| hsa-miR-548b-5p_st | 0.064886625 | 1.0 | 0.997663 | 0 |
| hsa-miR-454-star_st | 0.387939462 | 1.3 | 0.097588 | 0 |
| hsa-miR-455-3p_st | −0.256693419 | 1.2 | 0.751639 | 0 |
| hsa-miR-455-5p_st | −0.153420578 | 1.1 | 0.825595 | 0 |
| hsa-miR-466_st | 0.185178668 | 1.1 | 0.670737 | 0 |
| hsa-miR-483-3p_st | −0.189467908 | 1.1 | 0.707198 | 0 |
| hsa-miR-17_st | 0.11943726 | 1.1 | 0.905865 | 0 |
| hsa-miR-320d_st | −0.628783294 | 1.5 | 0.202497 | 0 |
| hsa-miR-485-3p_st | 0.338059737 | 1.3 | 0.340176 | 0 |
| hsa-miR-485-5p_st | 0.191608916 | 1.1 | 0.427966 | 0 |
| hsa-miR-486-3p_st | −0.17975227 | 1.1 | 0.480174 | 0 |
| hsa-miR-486-5p_st | −0.743273431 | 1.7 | 0.643843 | 0 |
| hsa-miR-644_st | 0.0194628 | 1.0 | 0.973767 | 0 |
| hsa-miR-3152_st | 0.24060656 | 1.2 | 0.520603 | 0 |
| hsa-miR-488_st | 0.126437783 | 1.1 | 0.699516 | 0 |
| hsa-miR-488-star_st | 0.041580865 | 1.0 | 0.40329 | 0 |
| hsa-miR-450a_st | −0.108702892 | 1.1 | 0.396352 | 0 |
| hsa-miR-20a_st | 0.344046068 | 1.3 | 0.495483 | 0 |
| hsa-miR-484_st | −0.500126791 | 1.4 | 0.135309 | 0 |
| hsa-miR-491-3p_st | 0.060504977 | 1.0 | 0.952596 | 0 |
| hsa-miR-491-5p_st | 0.030698298 | 1.0 | 0.584523 | 0 |
| hsa-miR-197_st | −0.696883063 | 1.6 | 0.044 | 1 |
| hsa-miR-493_st | 0.035999181 | 1.0 | 0.796909 | 0 |
| hsa-miR-493-star_st | 0.509258241 | 1.4 | 0.197965 | 0 |
| hsa-miR-494_st | −0.130398234 | 1.1 | 0.097488 | 0 |
| hsa-miR-495_st | 0.286205387 | 1.2 | 0.407756 | 0 |
| hsa-miR-496_st | −0.268513169 | 1.2 | 0.420812 | 0 |
| hsa-miR-497_st | −0.165608648 | 1.1 | 0.318572 | 0 |
| hsa-miR-497-star_st | 0.026392223 | 1.0 | 0.72276 | 0 |
| hsa-miR-498_st | −0.176676534 | 1.1 | 0.760928 | 0 |
| hsa-miR-499-3p_st | −0.023963772 | 1.0 | 0.287539 | 0 |
| hsa-miR-24-1-star_st | −0.247986316 | 1.2 | 0.40637 | 0 |
| hsa-miR-340_st | −0.064628232 | 1.0 | 0.475878 | 0 |
| hsa-miR-500b_st | 0.148753511 | 1.1 | 0.659832 | 0 |
| hsa-miR-500-star_st | 0.014843554 | 1.0 | 0.578955 | 0 |
| hsa-miR-501-3p_st | −0.007863155 | 1.0 | 0.794506 | 0 |
| hsa-miR-501-5p_st | −0.048432641 | 1.0 | 0.603738 | 0 |
| hsa-miR-502-3p_st | 0.001674817 | 1.0 | 0.373789 | 0 |
| hsa-miR-502-5p_st | 0.285542036 | 1.2 | 0.370688 | 0 |
| hsa-miR-503_st | −0.403490078 | 1.3 | 0.346126 | 0 |
| hsa-miR-192_st | 0.125650546 | 1.1 | 0.904627 | 0 |
| hsa-let-7g_st | −0.363501397 | 1.3 | 0.216764 | 0 |
| hsa-miR-505-star_st | 0.091171325 | 1.1 | 0.794287 | 0 |
| hsa-miR-506_st | −0.23767302 | 1.2 | 0.135341 | 0 |
| hsa-miR-507_st | −0.090093514 | 1.1 | 0.945494 | 0 |
| hsa-miR-508-3p_st | −0.147273458 | 1.1 | 0.359695 | 0 |
| hsa-miR-508-5p_st | 0.002413732 | 1.0 | 0.563613 | 0 |
| hsa-miR-509-3-5p_st | 0.570714998 | 1.5 | 0.14875 | 0 |
| hsa-miR-509-3p_st | 0.128220498 | 1.1 | 0.913518 | 0 |
| hsa-miR-106a_st | 0.101378491 | 1.1 | 0.946798 | 0 |
| hsa-miR-106b_st | 0.185179199 | 1.1 | 0.563126 | 0 |
| hsa-miR-511_st | 0.085185017 | 1.1 | 0.526099 | 0 |
| hsa-miR-512-3p_st | 0.064685789 | 1.0 | 0.354096 | 0 |
| hsa-miR-512-5p_st | 0.503000395 | 1.4 | 0.179486 | 0 |
| hsa-miR-513a-3p_st | −0.05657031 | 1.0 | 0.62999 | 0 |
| hsa-miR-23a_st | −0.434948759 | 1.4 | 0.212513 | 0 |
| hsa-miR-25_st | −0.185716551 | 1.1 | 0.46048 | 0 |
| hsa-miR-598_st | −0.191799391 | 1.1 | 0.513132 | 0 |
| hsa-miR-1260_st | −0.403205051 | 1.3 | 0.056879 | 0 |
| hsa-miR-514b-3p_st | 0.07425613 | 1.1 | 0.822853 | 0 |
| hsa-miR-514b-5p_st | 0.067846485 | 1.0 | 0.919044 | 0 |
| hsa-miR-515-3p_st | 0.084059464 | 1.1 | 0.865942 | 0 |
| hsa-miR-515-5p_st | −0.2143596 | 1.2 | 0.405633 | 0 |
| hsa-miR-516a-3p_st | −0.329152416 | 1.3 | 0.174738 | 0 |
| hsa-miR-27a_st | −0.008736244 | 1.0 | 0.552974 | 0 |
| hsa-miR-100_st | −0.700600673 | 1.6 | 0.140808 | 0 |
| hsa-miR-101_st | −0.005977347 | 1.0 | 0.945932 | 0 |
| hsa-miR-517a_st | 0.04727117 | 1.0 | 0.591028 | 0 |
| hsa-miR-517b_st | −0.089526955 | 1.1 | 0.633292 | 0 |
| hsa-miR-517c_st | −0.20363942 | 1.2 | 0.248041 | 0 |
| hsa-miR-517-star_st | 0.196586726 | 1.1 | 0.260688 | 0 |
| hsa-miR-518a-3p_st | 0.011247929 | 1.0 | 0.863137 | 0 |
| hsa-miR-518a-5p_st | 0.07097453 | 1.1 | 0.951435 | 0 |
| hsa-miR-518b_st | −0.16618584 | 1.1 | 0.805289 | 0 |
| hsa-miR-518c_st | −0.278928886 | 1.2 | 0.164114 | 0 |
| hsa-miR-301a_st | −0.245009462 | 1.2 | 0.403999 | 0 |
| hsa-miR-518d-3p_st | −0.211708439 | 1.2 | 0.439569 | 0 |
| hsa-miR-518d-5p_st | 0.1436207 | 1.1 | 0.668487 | 0 |
| hsa-miR-518e_st | −0.099638468 | 1.1 | 0.654467 | 0 |
| hsa-let-7f_st | −0.694731549 | 1.6 | 0.08495 | 0 |
| hsa-miR-518f_st | −0.05927946 | 1.0 | 0.835217 | 0 |
| hsa-miR-1826_st | −0.554018667 | 1.5 | 0.040821 | 1 |
| hsa-miR-519a_st | −0.219384895 | 1.2 | 0.352649 | 0 |
| hsa-miR-519a-star_st | 0.093514792 | 1.1 | 0.641192 | 0 |
| hsa-miR-519b-3p_st | −0.425182678 | 1.3 | 0.186583 | 0 |
| hsa-miR-1283_st | −0.155943273 | 1.1 | 0.371834 | 0 |
| hsa-miR-519c-3p_st | −0.22575524 | 1.2 | 0.264196 | 0 |
| hsa-miR-519c-5p_st | 0.154654271 | 1.1 | 0.111986 | 0 |
| hsa-miR-519d_st | −0.061202395 | 1.0 | 0.969483 | 0 |
| hsa-miR-519e_st | −0.192296554 | 1.1 | 0.199336 | 0 |
| hsa-miR-519e-star_st | 0.488619628 | 1.4 | 0.358655 | 0 |
| hsa-miR-520a-3p_st | 0.362513338 | 1.3 | 0.095464 | 0 |
| hsa-miR-1908_st | −0.575629916 | 1.5 | 0.046413 | 1 |
| hsa-miR-520b_st | −0.019244537 | 1.0 | 0.842801 | 0 |
| hsa-miR-520c-3p_st | −0.051537511 | 1.0 | 0.817038 | 0 |
| hsa-miR-520c-5p_st | 0.327211186 | 1.3 | 0.556497 | 0 |
| hsa-miR-181a-star_st | −0.263148392 | 1.2 | 0.210917 | 0 |
| hsa-miR-21_st | 1.051650156 | 2.1 | 0.388359 | 0 |
| hsa-miR-520e_st | −0.052259144 | 1.0 | 0.957308 | 0 |
| hsa-miR-520f_st | −0.046481814 | 1.0 | 0.714076 | 0 |
| hsa-miR-520g_st | 0.055817922 | 1.0 | 0.676425 | 0 |
| hsa-miR-520h_st | −0.208974208 | 1.2 | 0.914089 | 0 |
| hsa-miR-521_st | −0.158149096 | 1.1 | 0.934861 | 0 |
| hsa-miR-302a-star_st | 0.05672264 | 1.0 | 0.845147 | 0 |
| hsa-miR-886-3p_st | −0.578085603 | 1.5 | 0.312507 | 0 |
| hsa-miR-523_st | −0.264297496 | 1.2 | 0.438418 | 0 |
| hsa-miR-29a_st | −0.073408757 | 1.1 | 0.344176 | 0 |
| hsa-miR-524-3p_st | −0.050976935 | 1.0 | 0.961604 | 0 |
| hsa-miR-142-3p_st | 0.208816551 | 1.2 | 0.812056 | 0 |
| hsa-miR-525-3p_st | −0.382656793 | 1.3 | 0.359001 | 0 |
| hsa-miR-525-5p_st | −0.158809165 | 1.1 | 0.289635 | 0 |
| hsa-miR-762_st | −0.575847028 | 1.5 | 0.045985 | 1 |
| hsa-miR-423-3p_st | −0.548028656 | 1.5 | 0.048835 | 1 |
| hsa-miR-449b-star_st | 0.267454021 | 1.2 | 0.722372 | 0 |
| hsa-miR-527_st | 0.055650744 | 1.0 | 0.90262 | 0 |
| hsa-miR-532-3p_st | −0.539471701 | 1.5 | 0.19665 | 0 |
| hsa-miR-532-5p_st | −0.060405467 | 1.0 | 0.699909 | 0 |
| hsa-miR-539_st | 0.312546568 | 1.2 | 0.162657 | 0 |
| hsa-let-7a_st | −0.824508829 | 1.8 | 0.065088 | 0 |
| hsa-miR-541-star_st | −0.010741527 | 1.0 | 0.540966 | 0 |
| hsa-miR-542-3p_st | 0.509143648 | 1.4 | 0.475878 | 0 |
| hsa-miR-542-5p_st | −0.005662111 | 1.0 | 0.951076 | 0 |
| hsa-miR-543_st | −0.36666952 | 1.3 | 0.111372 | 0 |
| hsa-miR-544_st | −0.184340516 | 1.1 | 0.687404 | 0 |
| hsa-miR-544b_st | −0.029458818 | 1.0 | 0.870442 | 0 |
| hsa-miR-545_st | −0.275002227 | 1.2 | 0.333558 | 0 |
| hsa-miR-545-star_st | −0.302436296 | 1.2 | 0.277267 | 0 |
| hsa-miR-548a-3p_st | −0.368344887 | 1.3 | 0.3328 | 0 |
| hsa-miR-2861_st | −0.583304924 | 1.5 | 0.042577 | 1 |
| hsa-miR-31_st | 1.298151333 | 2.5 | 0.009757 | 1 |
| hsa-miR-367_st | −0.245383145 | 1.2 | 0.203076 | 0 |
| hsa-miR-548c-3p_st | −0.327843687 | 1.3 | 0.471866 | 0 |
| hsa-miR-548c-5p_st | 0.370434943 | 1.3 | 0.618749 | 0 |
| hsa-miR-548d-3p_st | −0.081789967 | 1.1 | 0.919378 | 0 |
| hsa-miR-548d-5p_st | −0.094209604 | 1.1 | 0.901321 | 0 |
| hsa-miR-548e_st | −0.073613468 | 1.1 | 0.773161 | 0 |
| hsa-miR-548f_st | −0.451095109 | 1.4 | 0.163708 | 0 |
| hsa-miR-548g_st | −0.435901558 | 1.4 | 0.273558 | 0 |
| hsa-miR-548h_st | 0.048735427 | 1.0 | 0.622131 | 0 |
| hsa-miR-548i_st | 0.072545848 | 1.1 | 0.910885 | 0 |
| hsa-miR-548j_st | 0.107960665 | 1.1 | 0.132578 | 0 |
| hsa-miR-548k_st | −0.171818706 | 1.1 | 0.375565 | 0 |
| hsa-miR-526b-star_st | −0.183676733 | 1.1 | 0.383082 | 0 |
| hsa-miR-548m_st | −0.036584983 | 1.0 | 0.975121 | 0 |
| hsa-miR-548n_st | −0.024294044 | 1.0 | 0.968557 | 0 |
| hsa-miR-548o_st | −0.194656099 | 1.1 | 0.36543 | 0 |
| hsa-miR-548p_st | −0.131374209 | 1.1 | 0.667308 | 0 |
| hsa-miR-548q_st | −0.016155569 | 1.0 | 0.986717 | 0 |
| hsa-miR-548s_st | −0.097270292 | 1.1 | 0.391382 | 0 |
| hsa-miR-548t_st | 0.119312425 | 1.1 | 0.590276 | 0 |
| hsa-miR-548u_st | −0.364220565 | 1.3 | 0.522679 | 0 |
| hsa-miR-638_st | −0.584682732 | 1.5 | 0.04193 | 1 |
| hsa-miR-548w_st | −0.037738308 | 1.0 | 0.961521 | 0 |
| hsa-miR-3119_st | −0.131144411 | 1.1 | 0.493569 | 0 |
| hsa-miR-549_st | −0.026759829 | 1.0 | 0.783934 | 0 |
| hsa-miR-205_st | −0.51443685 | 1.4 | 0.527071 | 0 |
| hsa-miR-550-star_st | 0.339898545 | 1.3 | 0.12366 | 0 |
| hsa-miR-551a_st | 0.200977616 | 1.1 | 0.658473 | 0 |
| hsa-miR-93_st | −0.221641861 | 1.2 | 0.341126 | 0 |
| hsa-miR-551b-star_st | −0.277100035 | 1.2 | 0.241495 | 0 |
| hsa-let-7d-star_st | −0.817870789 | 1.8 | 0.001487 | 1 |
| hsa-miR-553_st | −0.160633104 | 1.1 | 0.577204 | 0 |
| hsa-miR-554_st | −0.000795591 | 1.0 | 0.840677 | 0 |
| hsa-miR-555_st | −0.265055834 | 1.2 | 0.21244 | 0 |
| hsa-miR-556-3p_st | −0.256410658 | 1.2 | 0.312103 | 0 |
| hsa-miR-556-5p_st | −0.508436488 | 1.4 | 0.095273 | 0 |
| hsa-miR-557_st | −0.13120445 | 1.1 | 0.699336 | 0 |
| hsa-miR-558_st | −0.179270174 | 1.1 | 0.53685 | 0 |
| hsa-miR-559_st | −0.18621429 | 1.1 | 0.526963 | 0 |
| hsa-miR-561_st | −0.331311185 | 1.3 | 0.360479 | 0 |
| hsa-miR-562_st | −0.386126782 | 1.3 | 0.097783 | 0 |
| hsa-miR-563_st | −0.064099224 | 1.0 | 0.773148 | 0 |
| hsa-miR-361-5p_st | −0.459382135 | 1.4 | 0.171234 | 0 |
| hsa-miR-566_st | −0.069353282 | 1.0 | 0.705441 | 0 |
| hsa-miR-567_st | −0.044060323 | 1.0 | 0.935407 | 0 |
| hsa-miR-568_st | −0.467525779 | 1.4 | 0.126731 | 0 |
| hsa-miR-29c_st | −0.269385247 | 1.2 | 0.155476 | 0 |
| hsa-miR-570_st | 0.016769609 | 1.0 | 0.753979 | 0 |
| hsa-miR-571_st | −0.359236634 | 1.3 | 0.202981 | 0 |
| hsa-miR-572_st | −0.267871647 | 1.2 | 0.677986 | 0 |
| hsa-miR-573_st | −0.114798921 | 1.1 | 0.366426 | 0 |
| hsa-miR-132_st | 0.028551986 | 1.0 | 0.872063 | 0 |
| hsa-miR-574-5p_st | 0.052700792 | 1.0 | 0.582451 | 0 |
| hsa-let-7d_st | −0.813804506 | 1.8 | 0.073832 | 0 |
| hsa-miR-576-3p_st | −0.176403738 | 1.1 | 0.868782 | 0 |
| hsa-miR-576-5p_st | 0.240554893 | 1.2 | 0.346771 | 0 |
| hsa-miR-577_st | −0.113892876 | 1.1 | 0.73489 | 0 |
| hsa-miR-578_st | 0.043736811 | 1.0 | 0.596228 | 0 |
| hsa-miR-579_st | −0.229910494 | 1.2 | 0.074895 | 0 |
| hsa-miR-580_st | −0.409276349 | 1.3 | 0.245493 | 0 |
| hsa-miR-191_st | −0.421916212 | 1.3 | 0.232485 | 0 |
| hsa-miR-582-3p_st | −0.098230602 | 1.1 | 0.647968 | 0 |
| hsa-miR-582-5p_st | 0.643724428 | 1.6 | 0.291378 | 0 |
| hsa-miR-1915_st | −0.586936222 | 1.5 | 0.040942 | 1 |
| hsa-let-7c_st | −0.892685551 | 1.9 | 0.069965 | 0 |
| hsa-miR-585_st | 0.002859563 | 1.0 | 0.576827 | 0 |
| hsa-miR-586_st | −0.122085304 | 1.1 | 0.530982 | 0 |
| hsa-miR-324-5p_st | −0.443964939 | 1.4 | 0.259965 | 0 |
| hsa-miR-588_st | 0.14579447 | 1.1 | 0.261043 | 0 |
| hsa-miR-589_st | 0.27336546 | 1.2 | 0.341313 | 0 |
| hsa-miR-589-star_st | −0.030803597 | 1.0 | 0.77687 | 0 |
| hsa-miR-188-5p_st | −0.243711215 | 1.2 | 0.141976 | 0 |
| hsa-miR-103_st | −0.477432941 | 1.4 | 0.190041 | 0 |
| hsa-miR-591_st | 0.095416174 | 1.1 | 0.697 | 0 |
| hsa-miR-592_st | −0.072083362 | 1.1 | 0.993985 | 0 |
| hsa-miR-593_st | −0.017418176 | 1.0 | 0.724079 | 0 |
| hsa-miR-23b_st | −0.617010375 | 1.5 | 0.095369 | 0 |
| hsa-miR-595_st | −0.085356748 | 1.1 | 0.898469 | 0 |
| hsa-miR-99a_st | −0.731185897 | 1.7 | 0.108421 | 0 |
| hsa-miR-15b_st | −0.517761071 | 1.4 | 0.251774 | 0 |
| hsa-miR-135b_st | −0.596496063 | 1.5 | 0.058175 | 0 |
| hsa-miR-599_st | −0.323584821 | 1.3 | 0.156143 | 0 |
| hsa-miR-600_st | −0.514436629 | 1.4 | 0.072178 | 0 |
| hsa-miR-107_st | −0.447493933 | 1.4 | 0.135448 | 0 |
| hsa-miR-602_st | −0.261698324 | 1.2 | 0.637781 | 0 |
| hsa-miR-603_st | −0.247980993 | 1.2 | 0.2261 | 0 |
| hsa-miR-3065-5p_st | −0.380875659 | 1.3 | 0.07396 | 0 |
| hsa-miR-605_st | 0.641019851 | 1.6 | 0.324367 | 0 |
| hsa-miR-24_st | −0.369924977 | 1.3 | 0.297219 | 0 |
| hsa-miR-607_st | −0.209988467 | 1.2 | 0.436253 | 0 |
| hsa-miR-608_st | −0.124432034 | 1.1 | 0.876296 | 0 |
| hsa-miR-609_st | 0.276648148 | 1.2 | 0.272527 | 0 |
| hsa-miR-3196_st | −0.587337228 | 1.5 | 0.040807 | 1 |
| hsa-miR-26b_st | −0.384026001 | 1.3 | 0.230171 | 0 |
| hsa-miR-28-5p_st | −0.55681306 | 1.5 | 0.130808 | 0 |
| hsa-miR-613_st | 0.40788491 | 1.3 | 0.183099 | 0 |
| hsa-miR-338-3p_st | −0.569116127 | 1.5 | 0.154786 | 0 |
| hsa-miR-615-3p_st | 0.260529206 | 1.2 | 0.504159 | 0 |
| hsa-miR-454_st | −0.189181024 | 1.1 | 0.387638 | 0 |
| hsa-miR-616_st | 0.138119199 | 1.1 | 0.300393 | 0 |
| hsa-miR-151-5p_st | −0.642988953 | 1.6 | 0.175205 | 0 |
| hsa-miR-617_st | 0.264122497 | 1.2 | 0.724963 | 0 |
| hsa-miR-128_st | −0.529815342 | 1.4 | 0.297195 | 0 |
| hsa-miR-619_st | 0.047502326 | 1.0 | 0.713212 | 0 |
| hsa-miR-620_st | −0.089209372 | 1.1 | 0.543616 | 0 |
| hsa-miR-621_st | −0.215152202 | 1.2 | 0.499079 | 0 |
| hsa-let-7b_st | −1.060342624 | 2.1 | 0.056396 | 0 |
| hsa-miR-623_st | −0.273425861 | 1.2 | 0.678655 | 0 |
| hsa-miR-624_st | −0.200274361 | 1.1 | 0.216688 | 0 |
| hsa-miR-624-star_st | −0.289634627 | 1.2 | 0.164177 | 0 |
| hsa-miR-183_st | −0.837843653 | 1.8 | 0.007493 | 1 |
| hsa-miR-30b_st | −0.602168176 | 1.5 | 0.112342 | 0 |
| hsa-miR-626_st | −0.013604155 | 1.0 | 0.504051 | 0 |
| hsa-miR-627_st | −0.26285602 | 1.2 | 0.146501 | 0 |
| hsa-miR-628-3p_st | 0.20617255 | 1.2 | 0.947019 | 0 |
| hsa-miR-31-star_st | 1.008221295 | 2.0 | 0.10216 | 0 |
| hsa-miR-218-1-star_st | 0.19138655 | 1.1 | 0.736088 | 0 |
| hsa-miR-629-star_st | 0.322071604 | 1.3 | 0.364393 | 0 |
| hsa-miR-630_st | 0.241920486 | 1.2 | 0.789739 | 0 |
| hsa-miR-631_st | 0.083701709 | 1.1 | 0.841573 | 0 |
| hsa-miR-632_st | 0.274281817 | 1.2 | 0.823026 | 0 |
| hsa-miR-633_st | −0.180421076 | 1.1 | 0.172827 | 0 |
| hsa-miR-634_st | 0.349201484 | 1.3 | 0.97113 | 0 |
| hsa-miR-635_st | 0.278910187 | 1.2 | 0.301707 | 0 |
| hsa-miR-203_st | −0.603288584 | 1.5 | 0.233348 | 0 |
| hsa-miR-331-3p_st | −1.132529983 | 2.2 | 0.025408 | 1 |
| hsa-miR-125b_st | −1.068651203 | 2.1 | 0.020043 | 1 |
| hsa-let-7i_st | −0.696906788 | 1.6 | 0.113879 | 0 |
| hsa-miR-640_st | 0.238622431 | 1.2 | 0.418223 | 0 |
| hsa-miR-641_st | 0.21807405 | 1.2 | 0.22334 | 0 |
| hsa-miR-642_st | −0.007190351 | 1.0 | 0.707111 | 0 |
| hsa-miR-29b_st | −0.224465445 | 1.2 | 0.223127 | 0 |
| hsa-miR-122_st | −0.252753055 | 1.2 | 0.79053 | 0 |
| hsa-miR-645_st | 0.323059312 | 1.3 | 0.131863 | 0 |
| hsa-miR-646_st | −0.004106416 | 1.0 | 0.608446 | 0 |
| hsa-miR-647_st | 0.002310483 | 1.0 | 0.923629 | 0 |
| hsa-miR-648_st | −0.083495466 | 1.1 | 0.940363 | 0 |
| hsa-miR-649_st | 0.484824104 | 1.4 | 0.156254 | 0 |
| hsa-miR-182_st | −0.551647558 | 1.5 | 0.057179 | 0 |
| hsa-miR-651_st | 0.075278806 | 1.1 | 0.623722 | 0 |
| hsa-miR-652_st | −0.392665599 | 1.3 | 0.291985 | 0 |
| hsa-miR-653_st | −0.030142309 | 1.0 | 0.431318 | 0 |
| hsa-miR-654-3p_st | 0.204752724 | 1.2 | 0.494421 | 0 |
| hsa-miR-374b_st | −0.389219932 | 1.3 | 0.308202 | 0 |
| hsa-miR-130a_st | −0.598663032 | 1.5 | 0.551599 | 0 |
| hsa-miR-656_st | −0.264392133 | 1.2 | 0.230117 | 0 |
| hsa-miR-657_st | 0.47885415 | 1.4 | 0.053702 | 0 |
| hsa-miR-658_st | −0.192581941 | 1.1 | 0.956642 | 0 |
| hsa-miR-99b_st | −0.828597088 | 1.8 | 0.055557 | 0 |
| hsa-miR-660_st | 0.070805656 | 1.1 | 0.755811 | 0 |
| hsa-miR-661_st | −0.020490661 | 1.0 | 0.948768 | 0 |
| hsa-miR-30e-star_st | −0.972613242 | 2.0 | 0.089056 | 0 |
| hsa-miR-663_st | −0.46072759 | 1.4 | 0.115409 | 0 |
| hsa-miR-663b_st | 0.09342398 | 1.1 | 0.555169 | 0 |
| hsa-miR-664_st | −0.231788933 | 1.2 | 0.250493 | 0 |
| hsa-miR-664-star_st | −0.070920199 | 1.1 | 0.286971 | 0 |
| hsa-miR-30d_st | −0.903114559 | 1.9 | 0.084351 | 0 |
| hsa-miR-668_st | 0.24126713 | 1.2 | 0.36126 | 0 |
| hsa-miR-670_st | 0.144836769 | 1.1 | 0.494357 | 0 |
| hsa-miR-671-3p_st | 0.487730943 | 1.4 | 0.05442 | 0 |
| hsa-miR-671-5p_st | −0.132564406 | 1.1 | 0.76555 | 0 |
| hsa-miR-30a-star_st | −1.014525825 | 2.0 | 0.194213 | 0 |
| hsa-miR-675-star_st | −0.241334067 | 1.2 | 0.8568 | 0 |
| hsa-miR-148b_st | −1.02335761 | 2.0 | 0.049828 | 1 |
| hsa-miR-708_st | −0.304189926 | 1.2 | 0.249819 | 0 |
| hsa-miR-708-star_st | −0.151683145 | 1.1 | 0.559369 | 0 |
| hsa-miR-221-star_st | 0.912773185 | 1.9 | 0.437778 | 0 |
| hsa-let-7e_st | −0.984814682 | 2.0 | 0.064014 | 0 |
| hsa-miR-7-1-star_st | 0.492212494 | 1.4 | 0.539457 | 0 |
| hsa-miR-27b_st | −0.502423556 | 1.4 | 0.184995 | 0 |
| hsa-miR-34a_st | −0.254499795 | 1.2 | 0.306261 | 0 |
| hsa-miR-744_st | −0.12949049 | 1.1 | 0.710007 | 0 |
| hsa-miR-744-star_st | 0.231780028 | 1.2 | 0.848745 | 0 |
| hsa-miR-92b_st | −0.987018168 | 2.0 | 0.038841 | 1 |
| hsa-miR-759_st | −0.040744078 | 1.0 | 0.712175 | 0 |
| hsa-miR-760_st | −0.208336436 | 1.2 | 0.927732 | 0 |
| hsa-miR-92a_st | −0.851482509 | 1.8 | 0.114543 | 0 |
| hsa-miR-335_st | −0.790414042 | 1.7 | 0.080158 | 0 |
| hsa-miR-96_st | −0.755514489 | 1.7 | 0.074444 | 0 |
| hsa-miR-95_st | −1.101331298 | 2.1 | 0.038983 | 1 |
| hsa-miR-766_st | 0.661310372 | 1.6 | 0.271489 | 0 |
| hsa-miR-767-3p_st | −0.011061369 | 1.0 | 0.875187 | 0 |
| hsa-miR-767-5p_st | −0.063625523 | 1.0 | 0.968104 | 0 |
| hsa-miR-98_st | −1.038759536 | 2.1 | 0.05849 | 0 |
| hsa-miR-769-5p_st | 0.206252202 | 1.2 | 0.562884 | 0 |
| hsa-miR-770-5p_st | 0.143722508 | 1.1 | 0.446095 | 0 |
| hsa-miR-802_st | −0.215800339 | 1.2 | 0.257675 | 0 |
| hsa-miR-34b-star_st | −0.335682604 | 1.3 | 0.229502 | 0 |
| hsa-miR-581_st | −0.005040097 | 1.0 | 0.776021 | 0 |
| hsa-miR-181d_st | −0.884990285 | 1.8 | 0.01641 | 1 |
| hsa-miR-875-5p_st | −0.248276935 | 1.2 | 0.621334 | 0 |
| hsa-miR-876-3p_st | 0.129940501 | 1.1 | 0.426214 | 0 |
| hsa-miR-876-5p_st | −0.420448704 | 1.3 | 0.118189 | 0 |
| hsa-miR-151-3p_st | −0.68021113 | 1.6 | 0.10884 | 0 |
| hsa-miR-877-star_st | 0.560690237 | 1.5 | 0.166084 | 0 |
| hsa-miR-885-3p_st | −0.415912648 | 1.3 | 0.459231 | 0 |
| hsa-miR-200c_st | −1.325095773 | 2.5 | 0.020261 | 1 |
| hsa-miR-429_st | −0.164948557 | 1.1 | 0.846295 | 0 |
| hsa-miR-886-5p_st | −0.316774919 | 1.2 | 0.406905 | 0 |
| hsa-miR-224_st | −0.726924302 | 1.7 | 0.02415 | 1 |
| hsa-miR-888_st | 0.276258796 | 1.2 | 0.189061 | 0 |
| hsa-miR-4321_st | −0.007044479 | 1.0 | 0.969431 | 0 |
| hsa-miR-889_st | −0.05918416 | 1.0 | 0.852076 | 0 |
| hsa-miR-181b_st | −0.906378432 | 1.9 | 0.006273 | 1 |
| hsa-miR-891a_st | 0.199083711 | 1.1 | 0.928248 | 0 |
| hsa-miR-891b_st | −0.453899811 | 1.4 | 0.077047 | 0 |
| hsa-miR-892a_st | 0.220883626 | 1.2 | 0.923924 | 0 |
| hsa-miR-892b_st | 0.398921801 | 1.3 | 0.411778 | 0 |
| hsa-miR-9_st | −0.151862278 | 1.1 | 0.807469 | 0 |
| hsa-miR-222_st | 0.395156873 | 1.3 | 0.861048 | 0 |
| hsa-miR-141_st | −0.924987374 | 1.9 | 0.038222 | 1 |
| hsa-miR-221_st | 0.301407799 | 1.2 | 0.801187 | 0 |
| hsa-miR-924_st | 0.260584211 | 1.2 | 0.203996 | 0 |
| hsa-miR-181a_st | −0.98250849 | 2.0 | 0.011286 | 1 |
| hsa-miR-181c_st | −0.971885561 | 2.0 | 0.028918 | 1 |
| hsa-miR-4324_st | −1.277867506 | 2.4 | 0.020845 | 1 |
| hsa-miR-125a-5p_st | −1.489619943 | 2.8 | 0.005292 | 1 |
| hsa-miR-92b-star_st | −0.30561747 | 1.2 | 0.267503 | 0 |
| hsa-miR-3195_st | −0.084028146 | 1.1 | 0.970109 | 0 |
| hsa-miR-3137_st | −0.067024711 | 1.0 | 0.933037 | 0 |
| hsa-miR-934_st | −0.101763082 | 1.1 | 0.694268 | 0 |
| hsa-miR-935_st | −0.27814374 | 1.2 | 0.850873 | 0 |
| hsa-miR-551b_st | −0.118639181 | 1.1 | 0.335244 | 0 |
| hsa-miR-937_st | 0.272682005 | 1.2 | 0.6105 | 0 |
| hsa-miR-938_st | −0.006282307 | 1.0 | 0.972352 | 0 |
| hsa-miR-939_st | −0.168395898 | 1.1 | 0.747793 | 0 |
| hsa-miR-7-2-star_st | −1.572034886 | 3.0 | 0.003288 | 1 |
| hsa-miR-146b-5p_st | 1.926314015 | 3.8 | 0.00917 | 1 |
| hsa-miR-941_st | −0.127016526 | 1.1 | 0.727922 | 0 |
| hsa-miR-942_st | −0.016748552 | 1.0 | 0.736351 | 0 |
| hsa-miR-200a_st | −1.022937616 | 2.0 | 0.393213 | 0 |
| hsa-miR-944_st | −0.129469014 | 1.1 | 0.481936 | 0 |
| hsa-miR-7_st | −2.668505438 | 6.4 | 0.005751 | 1 |
| hsa-miR-375_st | 0.070149041 | 1.0 | 0.227761 | 0 |
| hsa-miR-96-star_st | −0.132706904 | 1.1 | 0.37858 | 0 |
| hsa-miR-424_st | −0.471546914 | 1.4 | 0.060324 | 0 |
| hsa-miR-181a-2-star_st | −0.427668902 | 1.3 | 0.507989 | 0 |
| hsa-miR-200b_st | −1.477637109 | 2.8 | 0.170971 | 0 |
| hsa-miR-146b-3p_st | 0.069042454 | 1.0 | 0.547276 | 0 |
| hsa-miR-99b-star_st | 0.011824216 | 1.0 | 0.981221 | 0 |
| hsa-miR-9-star_st | 0.66127744 | 1.6 | 0.052915 | 0 |
| | | Fold change | | |
| | Log2Diff (Hash | (Hash vs | Ttest (Hash vs | |
| Sample Name | vs Malignant) | Malignant) | Malignant | p < 0.05 |
| hsa-miR-150_st | 1.875714 | 3.7 | 0.044579 | 1 |
| hsa-let-7a-2-star_st | −0.31551 | 1.2 | 0.301611 | 0 |
| hsa-let-7a-star_st | −0.36659 | 1.3 | 0.265805 | 0 |
| hsa-miR-513b_st | 1.508771 | 2.8 | 0.00012 | 1 |
| hsa-let-7b-star_st | −0.44157 | 1.4 | 0.1364 | 0 |
| hsa-miR-593-star_st | 1.423566 | 2.7 | 0.0021 | 1 |
| hsa-let-7c-star_st | 0.089854 | 1.1 | 0.68784 | 0 |
| hsa-miR-516a-5p_st | 1.412039 | 2.7 | 0.009506 | 1 |
| hsa-miR-142-5p_st | 1.612655 | 3.1 | 0.088518 | 0 |
| hsa-miR-650_st | 1.332113 | 2.5 | 0.001228 | 1 |
| hsa-miR-513c_st | 1.315059 | 2.5 | 0.005088 | 1 |
| hsa-miR-761_st | 1.17578 | 2.3 | 0.006414 | 1 |
| hsa-let-7f-1-star_st | 0.210488 | 1.2 | 0.228788 | 0 |
| hsa-miR-513a-5p_st | 1.172649 | 2.3 | 0.000182 | 1 |
| hsa-miR-654-5p_st | 1.123106 | 2.2 | 0.007934 | 1 |
| hsa-let-7g-star_st | −0.03205 | 1.0 | 0.886417 | 0 |
| hsa-miR-3163_st | 1.117019 | 2.2 | 0.001389 | 1 |
| hsa-let-7i-star_st | −0.83393 | 1.8 | 0.090286 | 0 |
| hsa-miR-3165_st | 1.110869 | 2.2 | 0.005339 | 1 |
| hsa-miR-3194_st | 1.105484 | 2.2 | 1.27E−05 | 1 |
| hsa-miR-100-star_st | −0.28409 | 1.2 | 0.245667 | 0 |
| hsa-miR-4251_st | 1.105055 | 2.2 | 0.002238 | 1 |
| hsa-miR-101-star_st | −0.34338 | 1.3 | 0.276063 | 0 |
| hsa-miR-922_st | 1.102917 | 2.1 | 0.004213 | 1 |
| hsa-miR-103-2-star_st | −0.04052 | 1.0 | 0.883122 | 0 |
| hsa-miR-155_st | 1.026916 | 2.0 | 0.123084 | 0 |
| hsa-miR-105_st | −0.01722 | 1.0 | 0.955109 | 0 |
| hsa-miR-127-5p_st | 1.096208 | 2.1 | 0.004424 | 1 |
| hsa-miR-1286_st | 1.078866 | 2.1 | 0.000741 | 1 |
| hsa-miR-106a-star_st | −0.26819 | 1.2 | 0.373393 | 0 |
| hsa-miR-890_st | 1.061787 | 2.1 | 9.37E−07 | 1 |
| hsa-miR-3126-5p_st | 1.050997 | 2.1 | 0.000607 | 1 |
| hsa-miR-489_st | 1.047216 | 2.1 | 0.000714 | 1 |
| hsa-miR-10a_st | −0.94081 | 1.9 | 0.155039 | 0 |
| hsa-miR-10a-star_st | 0.157045 | 1.1 | 0.715294 | 0 |
| hsa-miR-10b_st | −1.26782 | 2.4 | 0.151474 | 0 |
| hsa-miR-10b-star_st | 0.127257 | 1.1 | 0.774825 | 0 |
| hsa-miR-1178_st | 0.273575 | 1.2 | 0.324821 | 0 |
| hsa-miR-1179_st | −0.2557 | 1.2 | 0.234035 | 0 |
| hsa-miR-1180_st | 0.11037 | 1.1 | 0.595952 | 0 |
| hsa-miR-1181_st | 0.849947 | 1.8 | 0.072693 | 0 |
| hsa-miR-3138_st | 1.042224 | 2.1 | 0.001262 | 1 |
| hsa-miR-220c_st | 1.040993 | 2.1 | 0.030176 | 1 |
| hsa-miR-510_st | 1.024315 | 2.0 | 0.005425 | 1 |
| hsa-miR-1185_st | −0.03782 | 1.0 | 0.909432 | 0 |
| hsa-miR-1193_st | 0.195471 | 1.1 | 0.25975 | 0 |
| hsa-miR-1197_st | −0.30152 | 1.2 | 0.078413 | 0 |
| hsa-miR-1200_st | 0.204895 | 1.2 | 0.378627 | 0 |
| hsa-miR-1201_st | −0.70374 | 1.6 | 0.330666 | 0 |
| hsa-miR-1202_st | 0.452039 | 1.4 | 0.080756 | 0 |
| hsa-miR-30c-1-star_st | 1.010031 | 2.0 | 0.000171 | 1 |
| hsa-miR-1204_st | 0.496589 | 1.4 | 0.145406 | 0 |
| hsa-miR-1205_st | 0.493938 | 1.4 | 0.211099 | 0 |
| hsa-miR-1206_st | −0.13518 | 1.1 | 0.677162 | 0 |
| hsa-miR-1207-3p_st | 0.027025 | 1.0 | 0.913751 | 0 |
| hsa-miR-1207-5p_st | 0.217031 | 1.2 | 0.35373 | 0 |
| hsa-miR-1208_st | 0.615208 | 1.5 | 0.064249 | 0 |
| hsa-miR-622_st | 1.002602 | 2.0 | 0.000496 | 1 |
| hsa-miR-1224-3p_st | 0.18884 | 1.1 | 0.44359 | 0 |
| hsa-miR-2116_st | 0.997903 | 2.0 | 0.043086 | 1 |
| hsa-miR-1225-3p_st | 0.275144 | 1.2 | 0.365143 | 0 |
| hsa-miR-1225-5p_st | 0.303019 | 1.2 | 0.165493 | 0 |
| hsa-miR-1226_st | 0.232875 | 1.2 | 0.572675 | 0 |
| hsa-miR-30b-star_st | 0.99662 | 2.0 | 0.000239 | 1 |
| hsa-miR-1227_st | 0.130739 | 1.1 | 0.722961 | 0 |
| hsa-miR-1295_st | 0.965171 | 2.0 | 0.037721 | 1 |
| hsa-miR-1228-star_st | −0.10986 | 1.1 | 0.684339 | 0 |
| hsa-miR-20b_st | −1.17916 | 2.3 | 0.066541 | 0 |
| hsa-miR-122-star_st | −0.21621 | 1.2 | 0.400662 | 0 |
| hsa-miR-1231_st | 0.176725 | 1.1 | 0.2744 | 0 |
| hsa-miR-1233_st | 0.309807 | 1.2 | 0.427353 | 0 |
| hsa-miR-524-5p_st | 0.962215 | 1.9 | 0.034037 | 1 |
| hsa-miR-1236_st | 0.392062 | 1.3 | 0.346635 | 0 |
| hsa-miR-873_st | 0.960328 | 1.9 | 0.023024 | 1 |
| hsa-miR-1238_st | 0.418415 | 1.3 | 0.089886 | 0 |
| hsa-miR-124_st | 0.301845 | 1.2 | 0.208797 | 0 |
| hsa-miR-1243_st | 0.216208 | 1.2 | 0.292643 | 0 |
| hsa-miR-1244_st | 0.273916 | 1.2 | 0.286377 | 0 |
| hsa-miR-1245_st | 0.014963 | 1.0 | 0.927859 | 0 |
| hsa-miR-3122_st | 0.956415 | 1.9 | 0.001031 | 1 |
| hsa-miR-134_st | 0.951455 | 1.9 | 0.008063 | 1 |
| hsa-miR-1248_st | 0.341599 | 1.3 | 0.525042 | 0 |
| hsa-miR-146a_st | −0.10185 | 1.1 | 0.886781 | 0 |
| hsa-miR-124-star_st | 0.188331 | 1.1 | 0.549282 | 0 |
| hsa-miR-1250_st | 0.477374 | 1.4 | 0.062697 | 0 |
| hsa-miR-764_st | 0.944549 | 1.9 | 0.029763 | 1 |
| hsa-miR-1252_st | −0.36086 | 1.3 | 0.0759 | 0 |
| hsa-miR-223_st | 0.345189 | 1.3 | 0.536099 | 0 |
| hsa-miR-34c-3p_st | 0.931121 | 1.9 | 0.027117 | 1 |
| hsa-miR-1255a_st | 0.544442 | 1.5 | 0.250369 | 0 |
| hsa-miR-1255b_st | 0.421079 | 1.3 | 0.225408 | 0 |
| hsa-miR-1256_st | −0.09835 | 1.1 | 0.765266 | 0 |
| hsa-miR-1257_st | 0.465829 | 1.4 | 0.244453 | 0 |
| hsa-miR-199b-5p_st | 1.498449 | 2.8 | 0.05722 | 0 |
| hsa-miR-1259_st | −0.58277 | 1.5 | 0.102356 | 0 |
| hsa-miR-492_st | 0.921568 | 1.9 | 0.004193 | 1 |
| hsa-miR-3132_st | 0.91529 | 1.9 | 0.006633 | 1 |
| hsa-miR-516b_st | 0.906583 | 1.9 | 0.01613 | 1 |
| hsa-miR-4294_st | 0.893861 | 1.9 | 0.011225 | 1 |
| hsa-miR-125b-2-star_st | 0.767035 | 1.7 | 0.076332 | 0 |
| hsa-miR-126_st | −0.82732 | 1.8 | 0.170127 | 0 |
| hsa-miR-198_st | 0.889231 | 1.9 | 0.001101 | 1 |
| hsa-miR-3191_st | 0.886404 | 1.8 | 0.004907 | 1 |
| hsa-miR-1261_st | 0.831512 | 1.8 | 0.085713 | 0 |
| hsa-miR-1262_st | 0.393495 | 1.3 | 0.364087 | 0 |
| hsa-miR-1263_st | −0.12747 | 1.1 | 0.552383 | 0 |
| hsa-miR-1264_st | 0.0838 | 1.1 | 0.688254 | 0 |
| hsa-miR-1265_st | −0.13493 | 1.1 | 0.554693 | 0 |
| hsa-miR-2114-star_st | 0.884951 | 1.8 | 9.46E−05 | 1 |
| hsa-miR-1267_st | 0.227677 | 1.2 | 0.357215 | 0 |
| hsa-miR-1268_st | 0.194238 | 1.1 | 0.49276 | 0 |
| hsa-miR-1269_st | 0.735073 | 1.7 | 0.096024 | 0 |
| hsa-miR-509-5p_st | 0.884033 | 1.8 | 0.022491 | 1 |
| hsa-miR-1270_st | 0.222275 | 1.2 | 0.288086 | 0 |
| hsa-miR-1271_st | 0.47179 | 1.4 | 0.060323 | 0 |
| hsa-miR-4303_st | 0.881737 | 1.8 | 0.016852 | 1 |
| hsa-miR-1273_st | 0.600535 | 1.5 | 0.06331 | 0 |
| hsa-miR-583_st | 0.877605 | 1.8 | 0.004025 | 1 |
| hsa-miR-1273d_st | 0.216905 | 1.2 | 0.549029 | 0 |
| hsa-miR-127-3p_st | −0.10559 | 1.1 | 0.848858 | 0 |
| hsa-miR-522-star_st | 0.873604 | 1.8 | 0.027286 | 1 |
| hsa-miR-214-star_st | 0.642526 | 1.6 | 0.202705 | 0 |
| hsa-miR-184_st | 0.871629 | 1.8 | 0.00286 | 1 |
| hsa-miR-614_st | 0.857975 | 1.8 | 0.04195 | 1 |
| hsa-miR-526b_st | 0.857611 | 1.8 | 0.04803 | 1 |
| hsa-miR-1277_st | −0.35282 | 1.3 | 0.280035 | 0 |
| hsa-miR-1278_st | −0.35407 | 1.3 | 0.071881 | 0 |
| hsa-miR-1279_st | −0.07028 | 1.0 | 0.771238 | 0 |
| hsa-miR-409-3p_st | 0.856455 | 1.8 | 0.007228 | 1 |
| hsa-miR-199a-5p_st | 0.427177 | 1.3 | 0.4469 | 0 |
| hsa-miR-1281_st | 0.591077 | 1.5 | 0.169969 | 0 |
| hsa-miR-1282_st | 0.687995 | 1.6 | 0.098686 | 0 |
| hsa-miR-33b-star_st | 0.856443 | 1.8 | 0.001443 | 1 |
| hsa-miR-1284_st | 0.527601 | 1.4 | 0.275269 | 0 |
| hsa-miR-1253_st | 0.855604 | 1.8 | 0.021967 | 1 |
| hsa-miR-584_st | 0.85471 | 1.8 | 0.012501 | 1 |
| hsa-miR-1287_st | 0.022292 | 1.0 | 0.933218 | 0 |
| hsa-miR-15a_st | −1.44424 | 2.7 | 0.062589 | 0 |
| hsa-miR-1289_st | 0.136444 | 1.1 | 0.728477 | 0 |
| hsa-miR-1290_st | −0.08536 | 1.1 | 0.70507 | 0 |
| hsa-miR-2117_st | 0.853282 | 1.8 | 0.006 | 1 |
| hsa-miR-1292_st | 0.567588 | 1.5 | 0.060194 | 0 |
| hsa-miR-1293_st | 0.4512 | 1.4 | 0.082812 | 0 |
| hsa-miR-129-3p_st | −0.33341 | 1.3 | 0.230296 | 0 |
| hsa-miR-520d-5p_st | 0.844618 | 1.8 | 0.01104 | 1 |
| hsa-miR-526a_st | 0.830069 | 1.8 | 0.000269 | 1 |
| hsa-miR-129-5p_st | 0.485606 | 1.4 | 0.145359 | 0 |
| hsa-miR-1254_st | 0.822758 | 1.8 | 0.018758 | 1 |
| hsa-miR-1297_st | −0.04232 | 1.0 | 0.899938 | 0 |
| hsa-miR-1298_st | 0.488409 | 1.4 | 0.260542 | 0 |
| hsa-miR-1299_st | 0.487699 | 1.4 | 0.26277 | 0 |
| hsa-miR-129-star_st | −0.10101 | 1.1 | 0.687767 | 0 |
| hsa-miR-1301_st | 0.252078 | 1.2 | 0.280775 | 0 |
| hsa-miR-19b_st | −1.05119 | 2.1 | 0.08905 | 0 |
| hsa-miR-1303_st | 0.250626 | 1.2 | 0.369998 | 0 |
| hsa-miR-1304_st | 0.305952 | 1.2 | 0.427725 | 0 |
| hsa-miR-1305_st | 0.400742 | 1.3 | 0.44183 | 0 |
| hsa-miR-550_st | 0.822385 | 1.8 | 0.001078 | 1 |
| hsa-miR-1307_st | 0.224586 | 1.2 | 0.406932 | 0 |
| hsa-miR-1308_st | −0.34814 | 1.3 | 0.152621 | 0 |
| hsa-miR-4291_st | −0.09111 | 1.1 | 0.602121 | 0 |
| hsa-miR-130a-star_st | 0.031171 | 1.0 | 0.871897 | 0 |
| hsa-miR-130b_st | −0.34378 | 1.3 | 0.206141 | 0 |
| hsa-miR-130b-star_st | −0.14432 | 1.1 | 0.543456 | 0 |
| hsa-miR-921_st | 0.821315 | 1.8 | 0.008626 | 1 |
| hsa-miR-520a-5p_st | 0.818193 | 1.8 | 0.012442 | 1 |
| hsa-miR-711_st | 0.817404 | 1.8 | 0.004764 | 1 |
| hsa-miR-1323_st | 0.674252 | 1.6 | 0.067574 | 0 |
| hsa-miR-1324_st | −0.22954 | 1.2 | 0.37862 | 0 |
| hsa-miR-490-3p_st | 0.816985 | 1.8 | 0.038855 | 1 |
| hsa-miR-718_st | 0.809921 | 1.8 | 0.017676 | 1 |
| hsa-miR-133b_st | −0.87168 | 1.8 | 0.075142 | 0 |
| hsa-miR-431-star_st | 0.807161 | 1.7 | 0.00016 | 1 |
| hsa-miR-552_st | 0.798145 | 1.7 | 0.047519 | 1 |
| hsa-miR-28-3p_st | −0.68249 | 1.6 | 0.14528 | 0 |
| hsa-miR-4265_st | 0.795713 | 1.7 | 0.009225 | 1 |
| hsa-miR-643_st | 0.791608 | 1.7 | 0.010439 | 1 |
| hsa-miR-136_st | −0.284 | 1.2 | 0.33253 | 0 |
| hsa-miR-136-star_st | −0.1462 | 1.1 | 0.566334 | 0 |
| hsa-miR-137_st | −0.29411 | 1.2 | 0.199558 | 0 |
| hsa-miR-138_st | −0.86137 | 1.8 | 0.158401 | 0 |
| hsa-miR-138-1-star_st | 0.327307 | 1.3 | 0.268606 | 0 |
| hsa-miR-138-2-star_st | −0.16997 | 1.1 | 0.26209 | 0 |
| hsa-miR-139-3p_st | 0.378296 | 1.3 | 0.240417 | 0 |
| hsa-miR-139-5p_st | −0.84982 | 1.8 | 0.269061 | 0 |
| hsa-miR-140-3p_st | −0.09719 | 1.1 | 0.735093 | 0 |
| hsa-miR-140-5p_st | −0.30577 | 1.2 | 0.644907 | 0 |
| hsa-miR-381_st | 0.790236 | 1.7 | 0.043949 | 1 |
| hsa-miR-141-star_st | −0.53573 | 1.4 | 0.08319 | 0 |
| hsa-miR-17-star_st | −0.65299 | 1.6 | 0.063488 | 0 |
| hsa-miR-4300_st | 0.786614 | 1.7 | 0.013676 | 1 |
| hsa-miR-143_st | −0.74859 | 1.7 | 0.205791 | 0 |
| hsa-miR-143-star_st | −0.95118 | 1.9 | 0.055095 | 0 |
| hsa-miR-144_st | −0.26459 | 1.2 | 0.295062 | 0 |
| hsa-miR-3198_st | 0.786366 | 1.7 | 0.000418 | 1 |
| hsa-miR-145_st | −0.9402 | 1.9 | 0.128782 | 0 |
| hsa-miR-145-star_st | −0.69784 | 1.6 | 0.191585 | 0 |
| hsa-miR-1468_st | 0.213869 | 1.2 | 0.658863 | 0 |
| hsa-miR-1469_st | −0.40619 | 1.3 | 0.192709 | 0 |
| hsa-miR-1973_st | 0.77824 | 1.7 | 0.036356 | 1 |
| hsa-miR-3154_st | 0.774015 | 1.7 | 0.01305 | 1 |
| hsa-miR-148a-star_st | 0.772736 | 1.7 | 0.0069 | 1 |
| hsa-miR-92a-1-star_st | 0.772103 | 1.7 | 0.036102 | 1 |
| hsa-miR-147_st | 0.078408 | 1.1 | 0.729668 | 0 |
| hsa-miR-194-star_st | 0.772062 | 1.7 | 0.041214 | 1 |
| hsa-miR-1471_st | 0.955038 | 1.9 | 0.065698 | 0 |
| hsa-miR-147b_st | 0.196952 | 1.1 | 0.331561 | 0 |
| hsa-miR-148a_st | −0.38789 | 1.3 | 0.447254 | 0 |
| hsa-miR-4299_st | 0.769782 | 1.7 | 0.003414 | 1 |
| hsa-miR-135a-star_st | 0.764534 | 1.7 | 0.02403 | 1 |
| hsa-miR-612_st | 0.764183 | 1.7 | 0.025795 | 1 |
| hsa-miR-1321_st | 0.763902 | 1.7 | 0.009257 | 1 |
| hsa-miR-149-star_st | −0.22187 | 1.2 | 0.445766 | 0 |
| hsa-miR-604_st | 0.758691 | 1.7 | 0.002329 | 1 |
| hsa-miR-150-star_st | 0.343949 | 1.3 | 0.122282 | 0 |
| hsa-miR-125b-1-star_st | 0.752057 | 1.7 | 0.007682 | 1 |
| hsa-miR-1183_st | 0.745688 | 1.7 | 0.002172 | 1 |
| hsa-miR-152_st | −0.875 | 1.8 | 0.192855 | 0 |
| hsa-miR-153_st | −0.44898 | 1.4 | 0.100604 | 0 |
| hsa-miR-1537_st | −0.41152 | 1.3 | 0.150367 | 0 |
| hsa-miR-1538_st | 0.063639 | 1.0 | 0.741651 | 0 |
| hsa-miR-1539_st | −0.00076 | 1.0 | 0.997692 | 0 |
| hsa-miR-154_st | 0.357909 | 1.3 | 0.380628 | 0 |
| hsa-miR-154-star_st | 0.710075 | 1.6 | 0.113858 | 0 |
| hsa-miR-432-star_st | 0.668255 | 1.6 | 0.108 | 0 |
| hsa-miR-155-star_st | −0.42433 | 1.3 | 0.103115 | 0 |
| hsa-miR-298_st | 0.743518 | 1.7 | 0.011212 | 1 |
| hsa-miR-15a-star_st | 0.607346 | 1.5 | 0.19181 | 0 |
| hsa-miR-2110_st | 0.739017 | 1.7 | 0.001131 | 1 |
| hsa-miR-15b-star_st | 0.054874 | 1.0 | 0.878563 | 0 |
| hsa-miR-1266_st | 0.737198 | 1.7 | 0.016896 | 1 |
| hsa-miR-16-1-star_st | −0.04953 | 1.0 | 0.806199 | 0 |
| hsa-miR-16-2-star_st | −0.18057 | 1.1 | 0.621224 | 0 |
| hsa-miR-490-5p_st | 0.733163 | 1.7 | 0.019749 | 1 |
| hsa-miR-19a_st | −0.73853 | 1.7 | 0.258325 | 0 |
| hsa-miR-3192_st | 0.725774 | 1.7 | 0.00695 | 1 |
| hsa-miR-3155_st | 0.724445 | 1.7 | 0.003758 | 1 |
| hsa-miR-187-star_st | 0.706712 | 1.6 | 0.009081 | 1 |
| hsa-miR-16_st | −1.03856 | 2.1 | 0.050954 | 0 |
| hsa-miR-936_st | 0.702428 | 1.6 | 0.045123 | 1 |
| hsa-miR-181c-star_st | 0.331128 | 1.3 | 0.204591 | 0 |
| hsa-miR-379_st | 0.701926 | 1.6 | 0.003104 | 1 |
| hsa-miR-370_st | 0.694164 | 1.6 | 0.041315 | 1 |
| hsa-miR-541_st | 0.69062 | 1.6 | 0.007675 | 1 |
| hsa-miR-518c-star_st | 0.689772 | 1.6 | 0.03014 | 1 |
| hsa-miR-487b_st | −1.10699 | 2.2 | 0.08351 | 0 |
| hsa-miR-182-star_st | −0.39898 | 1.3 | 0.092095 | 0 |
| hsa-miR-3121_st | 0.678557 | 1.6 | 0.039293 | 1 |
| hsa-miR-3190-5p_st | 0.671926 | 1.6 | 0.002554 | 1 |
| hsa-miR-1203_st | 0.671562 | 1.6 | 0.007305 | 1 |
| hsa-miR-185_st | −0.43336 | 1.4 | 0.116931 | 0 |
| hsa-miR-185-star_st | 0.459497 | 1.4 | 0.114769 | 0 |
| hsa-miR-4317_st | 0.670075 | 1.6 | 0.011878 | 1 |
| hsa-miR-186-star_st | 0.034947 | 1.0 | 0.893137 | 0 |
| hsa-miR-187_st | 0.128295 | 1.1 | 0.685571 | 0 |
| hsa-miR-4257_st | 0.668313 | 1.6 | 0.030443 | 1 |
| hsa-miR-188-3p_st | 0.1315 | 1.1 | 0.716928 | 0 |
| hsa-miR-1184_st | 0.665635 | 1.6 | 0.037958 | 1 |
| hsa-miR-18a_st | −0.28267 | 1.2 | 0.164215 | 0 |
| hsa-miR-18a-star_st | 0.342808 | 1.3 | 0.378501 | 0 |
| hsa-miR-3140_st | 0.747055 | 1.7 | 0.061304 | 0 |
| hsa-miR-18b-star_st | 0.216133 | 1.2 | 0.557616 | 0 |
| hsa-miR-190_st | −0.21498 | 1.2 | 0.439422 | 0 |
| hsa-miR-601_st | 0.660332 | 1.6 | 0.005155 | 1 |
| hsa-miR-1909_st | 0.192208 | 1.1 | 0.313762 | 0 |
| hsa-miR-342-5p_st | 0.042705 | 1.0 | 0.913482 | 0 |
| hsa-miR-575_st | 0.654796 | 1.6 | 0.008434 | 1 |
| hsa-miR-885-5p_st | 0.648065 | 1.6 | 0.000298 | 1 |
| hsa-miR-300_st | 0.646007 | 1.6 | 0.027217 | 1 |
| hsa-miR-1911_st | 0.301352 | 1.2 | 0.451263 | 0 |
| hsa-miR-1911-star_st | 0.556923 | 1.5 | 0.059267 | 0 |
| hsa-miR-1912_st | 0.166966 | 1.1 | 0.506126 | 0 |
| hsa-miR-4266_st | 0.644002 | 1.6 | 0.000275 | 1 |
| hsa-miR-1914_st | 0.018837 | 1.0 | 0.944043 | 0 |
| hsa-miR-3190-3p_st | 0.640988 | 1.6 | 0.00925 | 1 |
| hsa-miR-618_st | 0.488546 | 1.4 | 0.083241 | 0 |
| hsa-miR-1276_st | 0.640921 | 1.6 | 0.04047 | 1 |
| hsa-miR-191-star_st | 0.331398 | 1.3 | 0.296017 | 0 |
| hsa-miR-1258_st | 0.639865 | 1.6 | 0.006649 | 1 |
| hsa-miR-192-star_st | −0.2641 | 1.2 | 0.284911 | 0 |
| hsa-miR-193a-3p_st | −0.32757 | 1.3 | 0.342606 | 0 |
| hsa-miR-193a-5p_st | 0.183923 | 1.1 | 0.326434 | 0 |
| hsa-miR-3157_st | 0.638836 | 1.6 | 0.026165 | 1 |
| hsa-miR-193b-star_st | 0.331651 | 1.3 | 0.159696 | 0 |
| hsa-miR-194_st | −1.73238 | 3.3 | 0.062446 | 0 |
| hsa-miR-4307_st | 0.638766 | 1.6 | 0.01805 | 1 |
| hsa-miR-195_st | −0.80453 | 1.7 | 0.084874 | 0 |
| hsa-miR-610_st | 0.638641 | 1.6 | 0.028997 | 1 |
| hsa-miR-196a_st | 0.039663 | 1.0 | 0.93635 | 0 |
| hsa-miR-196a-star_st | 0.411119 | 1.3 | 0.270488 | 0 |
| hsa-miR-196b_st | −0.23934 | 1.2 | 0.237931 | 0 |
| hsa-miR-196b-star_st | 0.372442 | 1.3 | 0.052483 | 0 |
| hsa-let-7f-2-star_st | 0.318026 | 1.2 | 0.182124 | 0 |
| hsa-miR-1972_st | 0.380764 | 1.3 | 0.272945 | 0 |
| hsa-miR-3184_st | 0.637447 | 1.6 | 0.008355 | 1 |
| hsa-miR-523-star_st | 0.634662 | 1.6 | 0.014225 | 1 |
| hsa-miR-1976_st | 0.628612 | 1.5 | 0.13413 | 0 |
| hsa-miR-625-star_st | 0.268834 | 1.2 | 0.093935 | 0 |
| hsa-miR-587_st | 0.624556 | 1.5 | 0.026177 | 1 |
| hsa-miR-199a-3p_st | 0.292851 | 1.2 | 0.612147 | 0 |
| hsa-miR-1910_st | 0.609315 | 1.5 | 0.041325 | 1 |
| hsa-miR-199b-3p_st | 0.055928 | 1.0 | 0.924762 | 0 |
| hsa-miR-639_st | 0.60737 | 1.5 | 0.048683 | 1 |
| hsa-miR-1909-star_st | 0.606356 | 1.5 | 1.25E−06 | 1 |
| hsa-miR-19a-star_st | −0.27865 | 1.2 | 0.289921 | 0 |
| hsa-miR-29c-star_st | −1.6614 | 3.2 | 0.06327 | 0 |
| hsa-miR-940_st | 0.606168 | 1.5 | 0.042061 | 1 |
| hsa-miR-19b-2-star_st | −4.6E−05 | 1.0 | 0.999792 | 0 |
| hsa-miR-4269_st | 0.598367 | 1.5 | 0.012089 | 1 |
| hsa-miR-200a-star_st | −0.18332 | 1.1 | 0.477612 | 0 |
| hsa-miR-1182_st | 0.597921 | 1.5 | 0.016922 | 1 |
| hsa-miR-518f-star_st | 0.595153 | 1.5 | 1.56E−05 | 1 |
| hsa-miR-92a-2-star_st | 0.590639 | 1.5 | 0.011594 | 1 |
| hsa-miR-200c-star_st | −0.24667 | 1.2 | 0.477193 | 0 |
| hsa-miR-202_st | 0.641966 | 1.6 | 0.128031 | 0 |
| hsa-miR-202-star_st | −0.48909 | 1.4 | 0.150944 | 0 |
| hsa-miR-1914-star_st | 0.589626 | 1.5 | 0.005491 | 1 |
| hsa-miR-204_st | 0.102403 | 1.1 | 0.929327 | 0 |
| hsa-miR-665_st | 0.588756 | 1.5 | 0.005496 | 1 |
| hsa-miR-2052_st | −0.40226 | 1.3 | 0.188069 | 0 |
| hsa-miR-2053_st | −0.36242 | 1.3 | 0.300434 | 0 |
| hsa-miR-2054_st | −0.40666 | 1.3 | 0.439771 | 0 |
| hsa-miR-205-star_st | −0.08515 | 1.1 | 0.784191 | 0 |
| hsa-miR-206_st | 0.355367 | 1.3 | 0.320102 | 0 |
| hsa-miR-208a_st | −0.06721 | 1.0 | 0.678773 | 0 |
| hsa-miR-208b_st | −0.09144 | 1.1 | 0.568574 | 0 |
| hsa-miR-18b_st | −0.05011 | 1.0 | 0.766176 | 0 |
| hsa-miR-20a-star_st | −0.26139 | 1.2 | 0.701305 | 0 |
| hsa-miR-3181_st | 0.587489 | 1.5 | 0.001646 | 1 |
| hsa-miR-615-5p_st | 0.576959 | 1.5 | 0.005529 | 1 |
| hsa-miR-4259_st | 0.571165 | 1.5 | 0.042946 | 1 |
| hsa-miR-210_st | 0.411214 | 1.3 | 0.150148 | 0 |
| hsa-miR-211_st | 0.498095 | 1.4 | 0.26268 | 0 |
| hsa-miR-887_st | 0.561505 | 1.5 | 0.037889 | 1 |
| hsa-miR-2113_st | 0.697154 | 1.6 | 0.136226 | 0 |
| hsa-miR-2114_st | 0.411317 | 1.3 | 0.318544 | 0 |
| hsa-miR-943_st | 0.559797 | 1.5 | 0.047085 | 1 |
| hsa-miR-2115_st | −0.2913 | 1.2 | 0.21671 | 0 |
| hsa-miR-2115-star_st | 0.154402 | 1.1 | 0.639485 | 0 |
| hsa-miR-183-star_st | 0.555936 | 1.5 | 0.011356 | 1 |
| hsa-miR-2116-star_st | 0.31883 | 1.2 | 0.089494 | 0 |
| hsa-miR-920_st | 0.555817 | 1.5 | 0.023831 | 1 |
| hsa-miR-212_st | −0.05172 | 1.0 | 0.772202 | 0 |
| hsa-miR-214_st | 0.38808 | 1.3 | 0.175459 | 0 |
| hsa-miR-1825_st | 0.555623 | 1.5 | 0.043279 | 1 |
| hsa-miR-215_st | 0.214687 | 1.2 | 0.618197 | 0 |
| hsa-miR-216a_st | 0.119691 | 1.1 | 0.691055 | 0 |
| hsa-miR-195-star_st | 0.555205 | 1.5 | 0.039797 | 1 |
| hsa-miR-217_st | −0.3559 | 1.3 | 0.098919 | 0 |
| hsa-miR-23a-star_st | 0.553064 | 1.5 | 0.047762 | 1 |
| hsa-miR-25-star_st | 0.552629 | 1.5 | 0.006373 | 1 |
| hsa-miR-218-2-star_st | 0.337983 | 1.3 | 0.14847 | 0 |
| hsa-miR-1273c_st | 0.550118 | 1.5 | 3.29E−05 | 1 |
| hsa-miR-219-2-3p_st | 0.391086 | 1.3 | 0.136592 | 0 |
| hsa-miR-637_st | 0.548247 | 1.5 | 0.025514 | 1 |
| hsa-miR-21-star_st | 0.004585 | 1.0 | 0.985108 | 0 |
| hsa-miR-22_st | −1.30177 | 2.5 | 0.086492 | 0 |
| hsa-miR-220a_st | −0.30806 | 1.2 | 0.2524 | 0 |
| hsa-miR-220b_st | −0.3178 | 1.2 | 0.441672 | 0 |
| hsa-miR-219-1-3p_st | 0.547109 | 1.5 | 0.015326 | 1 |
| hsa-miR-4316_st | 0.545925 | 1.5 | 0.028937 | 1 |
| hsa-miR-877_st | 0.544226 | 1.5 | 0.024713 | 1 |
| hsa-miR-636_st | 0.542077 | 1.5 | 0.001797 | 1 |
| hsa-miR-3177_st | 0.54161 | 1.5 | 0.047178 | 1 |
| hsa-miR-1249_st | 0.363863 | 1.3 | 0.065578 | 0 |
| hsa-miR-223-star_st | −0.0065 | 1.0 | 0.977432 | 0 |
| hsa-miR-1224-5p_st | 0.540511 | 1.5 | 0.023547 | 1 |
| hsa-miR-564_st | 0.537952 | 1.5 | 0.017022 | 1 |
| hsa-miR-2276_st | 0.641524 | 1.6 | 0.05684 | 0 |
| hsa-miR-2277_st | 0.246085 | 1.2 | 0.05544 | 0 |
| hsa-miR-2278_st | 0.282229 | 1.2 | 0.380202 | 0 |
| hsa-miR-22-star_st | −1.5792 | 3.0 | 0.07011 | 0 |
| hsa-miR-2355_st | 0.332667 | 1.3 | 0.334732 | 0 |
| hsa-miR-769-3p_st | 0.528291 | 1.4 | 0.024697 | 1 |
| hsa-miR-1913_st | 0.524346 | 1.4 | 0.016332 | 1 |
| hsa-miR-1237_st | 0.523439 | 1.4 | 0.010516 | 1 |
| hsa-miR-1275_st | 0.519394 | 1.4 | 0.011609 | 1 |
| hsa-miR-30a_st | −1.59389 | 3.0 | 0.053482 | 0 |
| hsa-miR-20b-star_st | 0.516908 | 1.4 | 0.001255 | 1 |
| hsa-miR-24-2-star_st | −0.28328 | 1.2 | 0.49321 | 0 |
| hsa-miR-3173_st | 0.513023 | 1.4 | 0.005621 | 1 |
| hsa-miR-3148_st | 0.504822 | 1.4 | 0.017042 | 1 |
| hsa-miR-4271_st | 0.503092 | 1.4 | 0.016961 | 1 |
| hsa-miR-659_st | 0.502228 | 1.4 | 0.037237 | 1 |
| hsa-miR-26a-2-star_st | −0.53467 | 1.4 | 0.100688 | 0 |
| hsa-miR-1228_st | 0.492951 | 1.4 | 0.004204 | 1 |
| hsa-miR-26b-star_st | −0.91139 | 1.9 | 0.052335 | 0 |
| hsa-miR-3174_st | 0.488054 | 1.4 | 0.028285 | 1 |
| hsa-miR-27a-star_st | 0.365267 | 1.3 | 0.398611 | 0 |
| hsa-miR-1285_st | 0.48656 | 1.4 | 0.0276 | 1 |
| hsa-miR-483-5p_st | 0.484088 | 1.4 | 0.019715 | 1 |
| hsa-miR-1306_st | 0.47498 | 1.4 | 0.018178 | 1 |
| hsa-miR-431_st | −0.13576 | 1.1 | 0.480103 | 0 |
| hsa-miR-765_st | 0.469709 | 1.4 | 0.015836 | 1 |
| hsa-miR-2909_st | −0.41055 | 1.3 | 0.070524 | 0 |
| hsa-miR-296-3p_st | 0.454547 | 1.4 | 0.172491 | 0 |
| hsa-miR-296-5p_st | 0.030755 | 1.0 | 0.931089 | 0 |
| hsa-miR-297_st | 0.59143 | 1.5 | 0.076305 | 0 |
| hsa-miR-323b-5p_st | 0.467628 | 1.4 | 0.02754 | 1 |
| hsa-miR-299-3p_st | 0.527648 | 1.4 | 0.143613 | 0 |
| hsa-miR-299-5p_st | −0.3216 | 1.2 | 0.157633 | 0 |
| hsa-miR-487a_st | −0.24674 | 1.2 | 0.481717 | 0 |
| hsa-miR-29a-star_st | −0.03674 | 1.0 | 0.800392 | 0 |
| hsa-miR-625_st | 0.465514 | 1.4 | 0.001444 | 1 |
| hsa-miR-611_st | 0.463188 | 1.4 | 0.028433 | 1 |
| hsa-miR-3186-3p_st | 0.13237 | 1.1 | 0.464634 | 0 |
| hsa-miR-1294_st | 0.45925 | 1.4 | 0.000207 | 1 |
| hsa-miR-3147_st | 0.453551 | 1.4 | 0.033619 | 1 |
| hsa-miR-4285_st | 0.452341 | 1.4 | 0.005763 | 1 |
| hsa-miR-125a-3p_st | 0.449103 | 1.4 | 0.045559 | 1 |
| hsa-miR-301b_st | −0.25304 | 1.2 | 0.302497 | 0 |
| hsa-miR-302a_st | −0.12894 | 1.1 | 0.596222 | 0 |
| hsa-miR-518e-star_st | 0.38764 | 1.3 | 0.105122 | 0 |
| hsa-miR-302b_st | −0.52908 | 1.4 | 0.273314 | 0 |
| hsa-miR-302b-star_st | −0.04225 | 1.0 | 0.784851 | 0 |
| hsa-miR-302c_st | 0.199064 | 1.1 | 0.488595 | 0 |
| hsa-miR-302c-star_st | 0.29394 | 1.2 | 0.280373 | 0 |
| hsa-miR-302d_st | −0.06298 | 1.0 | 0.788555 | 0 |
| hsa-miR-302d-star_st | 0.175419 | 1.1 | 0.365582 | 0 |
| hsa-miR-302e_st | 0.214081 | 1.2 | 0.483859 | 0 |
| hsa-miR-302f_st | −0.30715 | 1.2 | 0.116968 | 0 |
| hsa-miR-3065-3p_st | 0.445283 | 1.4 | 0.381861 | 0 |
| hsa-miR-3130-5p_st | 0.443525 | 1.4 | 0.006041 | 1 |
| hsa-miR-3074_st | 0.361137 | 1.3 | 0.185526 | 0 |
| hsa-miR-1226-star_st | 0.430801 | 1.3 | 0.011928 | 1 |
| hsa-miR-3180-5p_st | 0.422603 | 1.3 | 0.004494 | 1 |
| hsa-miR-1229_st | −0.26277 | 1.2 | 0.265342 | 0 |
| hsa-miR-1272_st | 0.404677 | 1.3 | 0.025278 | 1 |
| hsa-miR-30c_st | −1.52878 | 2.9 | 0.055533 | 0 |
| hsa-miR-675_st | 0.403807 | 1.3 | 0.042038 | 1 |
| hsa-miR-30c-2-star_st | 0.193289 | 1.1 | 0.551076 | 0 |
| hsa-miR-1827_st | −0.25393 | 1.2 | 0.286345 | 0 |
| hsa-miR-30d-star_st | −0.35176 | 1.3 | 0.335497 | 0 |
| hsa-miR-30e_st | −1.35054 | 2.6 | 0.072977 | 0 |
| hsa-miR-1915-star_st | 0.394113 | 1.3 | 0.030865 | 1 |
| hsa-miR-1322_st | 0.284062 | 1.2 | 0.059485 | 0 |
| hsa-miR-3115_st | −0.1998 | 1.1 | 0.521976 | 0 |
| hsa-miR-3116_st | 0.710094 | 1.6 | 0.148728 | 0 |
| hsa-miR-3117_st | 0.098397 | 1.1 | 0.674454 | 0 |
| hsa-miR-3118_st | −0.38242 | 1.3 | 0.200637 | 0 |
| hsa-miR-519b-5p_st | 0.29833 | 1.2 | 0.124851 | 0 |
| hsa-miR-3120_st | 0.617442 | 1.5 | 0.161642 | 0 |
| hsa-miR-520d-3p_st | 0.391973 | 1.3 | 0.018418 | 1 |
| hsa-miR-326_st | 0.13226 | 1.1 | 0.709207 | 0 |
| hsa-miR-3123_st | −0.14352 | 1.1 | 0.647179 | 0 |
| hsa-miR-3124_st | 0.345081 | 1.3 | 0.100198 | 0 |
| hsa-miR-3125_st | 0.428808 | 1.3 | 0.073487 | 0 |
| hsa-miR-3126-3p_st | 0.297163 | 1.2 | 0.325054 | 0 |
| hsa-miR-1296_st | 0.383213 | 1.3 | 0.031933 | 1 |
| hsa-miR-3127_st | 0.573278 | 1.5 | 0.158541 | 0 |
| hsa-miR-3128_st | −0.04611 | 1.0 | 0.743651 | 0 |
| hsa-miR-3129_st | 0.032291 | 1.0 | 0.831513 | 0 |
| hsa-miR-3130-3p_st | 0.672925 | 1.6 | 0.066666 | 0 |
| hsa-miR-105-star_st | 0.375449 | 1.3 | 0.033706 | 1 |
| hsa-miR-3131_st | 0.465825 | 1.4 | 0.107269 | 0 |
| hsa-miR-4255_st | 0.365234 | 1.3 | 0.003793 | 1 |
| hsa-miR-3133_st | −0.0711 | 1.1 | 0.814963 | 0 |
| hsa-miR-3134_st | 0.238194 | 1.2 | 0.249768 | 0 |
| hsa-miR-3135_st | 0.570109 | 1.5 | 0.19266 | 0 |
| hsa-miR-3136_st | 0.239942 | 1.2 | 0.379858 | 0 |
| hsa-miR-19b-1-star_st | −0.34712 | 1.3 | 0.091626 | 0 |
| hsa-miR-1247_st | 0.360962 | 1.3 | 0.048596 | 1 |
| hsa-miR-3139_st | 0.0296 | 1.0 | 0.814292 | 0 |
| hsa-miR-596_st | 0.34814 | 1.3 | 0.010696 | 1 |
| hsa-miR-3141_st | 0.179654 | 1.1 | 0.428193 | 0 |
| hsa-miR-3142_st | −0.39884 | 1.3 | 0.275076 | 0 |
| hsa-miR-1302_st | −0.03991 | 1.0 | 0.828261 | 0 |
| hsa-miR-3144-3p_st | 0.493278 | 1.4 | 0.335096 | 0 |
| hsa-miR-3144-5p_st | 0.654076 | 1.6 | 0.241783 | 0 |
| hsa-miR-3145_st | 0.102562 | 1.1 | 0.775374 | 0 |
| hsa-miR-3146_st | 0.354102 | 1.3 | 0.29619 | 0 |
| hsa-miR-346_st | 0.340806 | 1.3 | 0.015166 | 1 |
| hsa-miR-4256_st | 0.337694 | 1.3 | 0.034184 | 1 |
| hsa-miR-3149_st | 0.174477 | 1.1 | 0.542794 | 0 |
| hsa-miR-3150_st | 0.328084 | 1.3 | 0.245074 | 0 |
| hsa-miR-3151_st | 0.431145 | 1.3 | 0.061934 | 0 |
| hsa-miR-1470_st | 0.2901 | 1.2 | 0.04659 | 1 |
| hsa-miR-3153_st | 0.608063 | 1.5 | 0.071465 | 0 |
| hsa-miR-514_st | −0.32643 | 1.3 | 0.018998 | 1 |
| hsa-miR-3175_st | 0.061548 | 1.0 | 0.458648 | 0 |
| hsa-miR-3156_st | 0.039395 | 1.0 | 0.827998 | 0 |
| hsa-miR-504_st | −0.38157 | 1.3 | 0.039504 | 1 |
| hsa-miR-3158_st | 0.337955 | 1.3 | 0.324134 | 0 |
| hsa-miR-3159_st | 0.375088 | 1.3 | 0.193 | 0 |
| hsa-miR-3160_st | 0.52557 | 1.4 | 0.112793 | 0 |
| hsa-miR-3161_st | 0.484887 | 1.4 | 0.05837 | 0 |
| hsa-miR-3162_st | 0.398542 | 1.3 | 0.081205 | 0 |
| hsa-miR-1234_st | 0.140992 | 1.1 | 0.339242 | 0 |
| hsa-miR-3164_st | 0.981603 | 2.0 | 0.061762 | 0 |
| hsa-miR-516b-star_st | −0.40417 | 1.3 | 0.012774 | 1 |
| hsa-miR-3166_st | −0.29905 | 1.2 | 0.292792 | 0 |
| hsa-miR-4327_st | 0.435291 | 1.4 | 0.072588 | 0 |
| hsa-miR-3168_st | −0.34 | 1.3 | 0.105811 | 0 |
| hsa-miR-3169_st | 0.243496 | 1.2 | 0.162131 | 0 |
| hsa-miR-3170_st | 0.654847 | 1.6 | 0.066308 | 0 |
| hsa-miR-3171_st | −0.24959 | 1.2 | 0.316457 | 0 |
| hsa-miR-3172_st | −0.72679 | 1.7 | 0.090613 | 0 |
| hsa-miR-374b-star_st | −0.42979 | 1.3 | 0.019 | 1 |
| hsa-miR-34c-5p_st | −0.45892 | 1.4 | 0.036567 | 1 |
| hsa-miR-758_st | −0.46021 | 1.4 | 0.037008 | 1 |
| hsa-miR-3176_st | −0.34295 | 1.3 | 0.178088 | 0 |
| hsa-miR-606_st | −0.46641 | 1.4 | 0.001349 | 1 |
| hsa-miR-3178_st | 0.042974 | 1.0 | 0.875195 | 0 |
| hsa-miR-3179_st | 0.214452 | 1.2 | 0.674335 | 0 |
| hsa-miR-3180-3p_st | 0.100646 | 1.1 | 0.626711 | 0 |
| hsa-miR-597_st | −0.48867 | 1.4 | 0.00307 | 1 |
| hsa-miR-148b-star_st | −0.49185 | 1.4 | 0.000147 | 1 |
| hsa-miR-3182_st | 0.050587 | 1.0 | 0.872925 | 0 |
| hsa-miR-3183_st | 0.475639 | 1.4 | 0.101116 | 0 |
| hsa-miR-144-star_st | −0.5221 | 1.4 | 0.020296 | 1 |
| hsa-miR-3185_st | 0.057419 | 1.0 | 0.814649 | 0 |
| hsa-miR-4270_st | 0.101124 | 1.1 | 0.709515 | 0 |
| hsa-miR-3186-5p_st | 0.457756 | 1.4 | 0.257013 | 0 |
| hsa-miR-3187_st | 0.431073 | 1.3 | 0.076258 | 0 |
| hsa-miR-3188_st | 0.389334 | 1.3 | 0.154408 | 0 |
| hsa-miR-3189_st | 0.563296 | 1.5 | 0.144408 | 0 |
| hsa-miR-548b-3p_st | −0.52461 | 1.4 | 0.046675 | 1 |
| hsa-miR-99a-star_st | −0.53393 | 1.4 | 0.032903 | 1 |
| hsa-miR-376a-star_st | −0.53981 | 1.5 | 0.039221 | 1 |
| hsa-miR-216b_st | −0.54189 | 1.5 | 0.048686 | 1 |
| hsa-miR-3193_st | 0.214962 | 1.2 | 0.385821 | 0 |
| hsa-miR-548v_st | −0.54296 | 1.5 | 0.000842 | 1 |
| hsa-miR-26a-1-star_st | 0.219347 | 1.2 | 0.447431 | 0 |
| hsa-miR-1291_st | 0.050424 | 1.0 | 0.832165 | 0 |
| hsa-miR-3197_st | 0.296022 | 1.2 | 0.267736 | 0 |
| hsa-miR-629_st | −0.57916 | 1.5 | 0.00683 | 1 |
| hsa-miR-3199_st | 0.233721 | 1.2 | 0.395209 | 0 |
| hsa-miR-190b_st | −0.5809 | 1.5 | 0.002315 | 1 |
| hsa-miR-32_st | −0.76384 | 1.7 | 0.076709 | 0 |
| hsa-miR-3200_st | −0.51984 | 1.4 | 0.163318 | 0 |
| hsa-miR-3201_st | 0.200403 | 1.1 | 0.485845 | 0 |
| hsa-miR-3202_st | 0.557603 | 1.5 | 0.103653 | 0 |
| hsa-miR-320a_st | −0.28228 | 1.2 | 0.242213 | 0 |
| hsa-miR-4289_st | −0.59249 | 1.5 | 0.010417 | 1 |
| hsa-miR-655_st | −0.60014 | 1.5 | 0.029042 | 1 |
| hsa-miR-320b_st | −0.60324 | 1.5 | 0.000494 | 1 |
| hsa-miR-219-5p_st | −0.60391 | 1.5 | 0.026049 | 1 |
| hsa-miR-323-3p_st | 0.023479 | 1.0 | 0.894121 | 0 |
| hsa-miR-323-5p_st | 0.637984 | 1.6 | 0.061775 | 0 |
| hsa-miR-323b-3p_st | 0.080146 | 1.1 | 0.648082 | 0 |
| hsa-miR-103-as_st | −0.61739 | 1.5 | 0.00099 | 1 |
| hsa-miR-424-star_st | −0.61812 | 1.5 | 0.003543 | 1 |
| hsa-miR-33a-star_st | −0.63921 | 1.6 | 0.045775 | 1 |
| hsa-miR-325_st | 0.39161 | 1.3 | 0.202669 | 0 |
| hsa-miR-590-3p_st | −0.3489 | 1.3 | 0.223918 | 0 |
| hsa-miR-328_st | −1.11995 | 2.2 | 0.066768 | 0 |
| hsa-miR-329_st | −0.21775 | 1.2 | 0.568575 | 0 |
| hsa-miR-32-star_st | 0.332362 | 1.3 | 0.231181 | 0 |
| hsa-miR-330-3p_st | 0.27184 | 1.2 | 0.284543 | 0 |
| hsa-miR-330-5p_st | 0.306067 | 1.2 | 0.261057 | 0 |
| hsa-miR-146a-star_st | −0.66904 | 1.6 | 0.020381 | 1 |
| hsa-miR-331-5p_st | −0.2209 | 1.2 | 0.287661 | 0 |
| hsa-miR-3167_st | −0.0953 | 1.1 | 0.669109 | 0 |
| hsa-miR-335-star_st | 0.382073 | 1.3 | 0.292268 | 0 |
| hsa-miR-337-3p_st | 0.685706 | 1.6 | 0.19338 | 0 |
| hsa-miR-337-5p_st | 0.056412 | 1.0 | 0.81568 | 0 |
| hsa-miR-421_st | −0.67225 | 1.6 | 0.000691 | 1 |
| hsa-miR-338-5p_st | −0.37539 | 1.3 | 0.166568 | 0 |
| hsa-miR-339-3p_st | −0.33611 | 1.3 | 0.297066 | 0 |
| hsa-miR-548a-5p_st | −0.67355 | 1.6 | 0.003185 | 1 |
| hsa-miR-33a_st | −0.34041 | 1.3 | 0.322089 | 0 |
| hsa-miR-590-5p_st | −0.69213 | 1.6 | 0.024674 | 1 |
| hsa-miR-33b_st | −0.3991 | 1.3 | 0.291439 | 0 |
| hsa-miR-616-star_st | −0.70233 | 1.6 | 0.001223 | 1 |
| hsa-miR-569_st | −0.3296 | 1.3 | 0.192037 | 0 |
| hsa-miR-340-star_st | −0.40199 | 1.3 | 0.193664 | 0 |
| hsa-miR-342-3p_st | −0.10668 | 1.1 | 0.852719 | 0 |
| hsa-miR-135b-star_st | 0.046316 | 1.0 | 0.838353 | 0 |
| hsa-miR-345_st | −1.27999 | 2.4 | 0.149817 | 0 |
| hsa-miR-1288_st | 0.393475 | 1.3 | 0.280707 | 0 |
| hsa-miR-135a_st | −0.71224 | 1.6 | 0.000328 | 1 |
| hsa-miR-34a-star_st | −1.00721 | 2.0 | 0.141062 | 0 |
| hsa-miR-34b_st | 0.412819 | 1.3 | 0.214896 | 0 |
| hsa-miR-4288_st | −0.71691 | 1.6 | 0.003787 | 1 |
| hsa-miR-222-star_st | −0.74306 | 1.7 | 0.016909 | 1 |
| hsa-miR-933_st | −0.76174 | 1.7 | 0.000268 | 1 |
| hsa-miR-361-3p_st | −1.08364 | 2.1 | 0.10538 | 0 |
| hsa-miR-224-star_st | −0.76381 | 1.7 | 0.005614 | 1 |
| hsa-miR-362-3p_st | 0.006796 | 1.0 | 0.988151 | 0 |
| hsa-miR-362-5p_st | −0.72667 | 1.7 | 0.054596 | 0 |
| hsa-miR-363_st | 0.443007 | 1.4 | 0.41525 | 0 |
| hsa-miR-363-star_st | 0.579354 | 1.5 | 0.057523 | 0 |
| hsa-miR-1979_st | −0.78826 | 1.7 | 0.025769 | 1 |
| hsa-miR-365-star_st | 0.445629 | 1.4 | 0.053414 | 0 |
| hsa-miR-548x_st | −0.45802 | 1.4 | 0.095962 | 0 |
| hsa-miR-367-star_st | −0.23215 | 1.2 | 0.359146 | 0 |
| hsa-miR-369-3p_st | −0.15192 | 1.1 | 0.557067 | 0 |
| hsa-miR-369-5p_st | −0.35635 | 1.3 | 0.101355 | 0 |
| hsa-miR-548l_st | 0.419119 | 1.3 | 0.18952 | 0 |
| hsa-miR-371-3p_st | 0.207185 | 1.2 | 0.451694 | 0 |
| hsa-miR-371-5p_st | 0.437153 | 1.4 | 0.220173 | 0 |
| hsa-miR-372_st | 0.693872 | 1.6 | 0.123654 | 0 |
| hsa-miR-373_st | 0.254604 | 1.2 | 0.239647 | 0 |
| hsa-miR-373-star_st | 0.512566 | 1.4 | 0.141115 | 0 |
| hsa-miR-374a_st | −0.79374 | 1.7 | 0.118177 | 0 |
| hsa-miR-374a-star_st | −0.59143 | 1.5 | 0.092059 | 0 |
| hsa-miR-500_st | −0.80128 | 1.7 | 0.001245 | 1 |
| hsa-miR-320c_st | −0.81423 | 1.8 | 0.005592 | 1 |
| hsa-miR-875-3p_st | 0.061773 | 1.0 | 0.773593 | 0 |
| hsa-miR-376a_st | 0.227783 | 1.2 | 0.524136 | 0 |
| hsa-miR-29b-1-star_st | −0.82186 | 1.8 | 0.001095 | 1 |
| hsa-miR-376b_st | −0.28092 | 1.2 | 0.185203 | 0 |
| hsa-miR-376c_st | 0.025575 | 1.0 | 0.97473 | 0 |
| hsa-miR-377_st | 0.048302 | 1.0 | 0.839812 | 0 |
| hsa-miR-377-star_st | −0.02002 | 1.0 | 0.955863 | 0 |
| hsa-miR-378_st | −0.13188 | 1.1 | 0.521252 | 0 |
| hsa-miR-378b_st | 0.277363 | 1.2 | 0.236973 | 0 |
| hsa-miR-378c_st | 0.200218 | 1.1 | 0.280367 | 0 |
| hsa-miR-378-star_st | −0.27132 | 1.2 | 0.395511 | 0 |
| hsa-miR-662_st | −0.83351 | 1.8 | 0.000127 | 1 |
| hsa-miR-379-star_st | −0.26992 | 1.2 | 0.317944 | 0 |
| hsa-miR-380_st | −0.26478 | 1.2 | 0.260717 | 0 |
| hsa-miR-380-star_st | 0.341074 | 1.3 | 0.152083 | 0 |
| hsa-miR-1274b_st | −0.85283 | 1.8 | 0.001466 | 1 |
| hsa-miR-382_st | 0.171209 | 1.1 | 0.549609 | 0 |
| hsa-miR-383_st | 0.561393 | 1.5 | 0.176321 | 0 |
| hsa-miR-384_st | −0.50449 | 1.4 | 0.132072 | 0 |
| hsa-miR-126-star_st | −0.44699 | 1.4 | 0.071867 | 0 |
| hsa-miR-409-5p_st | −0.18055 | 1.1 | 0.186087 | 0 |
| hsa-miR-410_st | −0.1086 | 1.1 | 0.436408 | 0 |
| hsa-miR-411_st | 0.231502 | 1.2 | 0.513814 | 0 |
| hsa-miR-411-star_st | 0.09155 | 1.1 | 0.712877 | 0 |
| hsa-miR-412_st | 0.292108 | 1.2 | 0.367269 | 0 |
| hsa-miR-3143_st | −0.22515 | 1.2 | 0.404125 | 0 |
| hsa-miR-422a_st | 0.090633 | 1.1 | 0.527905 | 0 |
| hsa-miR-628-5p_st | −0.86805 | 1.8 | 0.002935 | 1 |
| hsa-miR-423-5p_st | 0.302279 | 1.2 | 0.072829 | 0 |
| hsa-miR-27b-star_st | −0.90672 | 1.9 | 0.00456 | 1 |
| hsa-miR-499-5p_st | −0.90952 | 1.9 | 6.77E−05 | 1 |
| hsa-miR-425_st | −1.32873 | 2.5 | 0.086668 | 0 |
| hsa-miR-193b_st | −0.9194 | 1.9 | 0.044117 | 1 |
| hsa-miR-4252_st | 0.250581 | 1.2 | 0.33181 | 0 |
| hsa-miR-4253_st | 0.340679 | 1.3 | 0.119376 | 0 |
| hsa-miR-4254_st | −0.11969 | 1.1 | 0.66528 | 0 |
| hsa-miR-324-3p_st | −0.95255 | 1.9 | 0.00081 | 1 |
| hsa-miR-4329_st | −0.97187 | 2.0 | 0.001264 | 1 |
| hsa-miR-1251_st | −0.97729 | 2.0 | 0.002066 | 1 |
| hsa-miR-4258_st | 0.383606 | 1.3 | 0.359047 | 0 |
| hsa-miR-1274a_st | −0.99078 | 2.0 | 0.018219 | 1 |
| hsa-miR-425-star_st | 0.318846 | 1.2 | 0.218195 | 0 |
| hsa-miR-4260_st | 0.444888 | 1.4 | 0.210779 | 0 |
| hsa-miR-4261_st | 0.335569 | 1.3 | 0.095322 | 0 |
| hsa-miR-4262_st | 0.15299 | 1.1 | 0.615328 | 0 |
| hsa-miR-4263_st | 0.188585 | 1.1 | 0.131973 | 0 |
| hsa-miR-4264_st | −0.08916 | 1.1 | 0.679251 | 0 |
| hsa-miR-1280_st | −0.99181 | 2.0 | 0.028722 | 1 |
| hsa-miR-132-star_st | −1.00618 | 2.0 | 0.020822 | 1 |
| hsa-miR-4267_st | −0.10366 | 1.1 | 0.790937 | 0 |
| hsa-miR-4268_st | −0.17902 | 1.1 | 0.501817 | 0 |
| hsa-miR-133a_st | −1.01968 | 2.0 | 0.029363 | 1 |
| hsa-miR-888-star_st | −0.02584 | 1.0 | 0.950438 | 0 |
| hsa-miR-1260b_st | −1.02941 | 2.0 | 0.012167 | 1 |
| hsa-miR-4272_st | −0.30521 | 1.2 | 0.292354 | 0 |
| hsa-miR-4273_st | 0.140959 | 1.1 | 0.456688 | 0 |
| hsa-miR-4274_st | −0.02513 | 1.0 | 0.908477 | 0 |
| hsa-miR-4275_st | −0.61281 | 1.5 | 0.063197 | 0 |
| hsa-miR-4276_st | −0.28848 | 1.2 | 0.124878 | 0 |
| hsa-miR-4277_st | 0.136556 | 1.1 | 0.43648 | 0 |
| hsa-miR-4278_st | 0.205645 | 1.2 | 0.299545 | 0 |
| hsa-miR-4279_st | 0.266617 | 1.2 | 0.447893 | 0 |
| hsa-miR-4280_st | 0.605559 | 1.5 | 0.186328 | 0 |
| hsa-miR-4281_st | −0.2498 | 1.2 | 0.397618 | 0 |
| hsa-miR-4282_st | −0.29165 | 1.2 | 0.149744 | 0 |
| hsa-miR-4283_st | 0.511803 | 1.4 | 0.059597 | 0 |
| hsa-miR-4284_st | −0.40301 | 1.3 | 0.233105 | 0 |
| hsa-miR-23b-star_st | −1.04182 | 2.1 | 0.019091 | 1 |
| hsa-miR-574-3p_st | −1.04489 | 2.1 | 0.021388 | 1 |
| hsa-miR-4287_st | 0.649212 | 1.6 | 0.2742 | 0 |
| hsa-miR-320e_st | −1.04849 | 2.1 | 0.023436 | 1 |
| hsa-miR-339-5p_st | −1.05351 | 2.1 | 0.036858 | 1 |
| hsa-miR-505_st | −1.05709 | 2.1 | 0.047437 | 1 |
| hsa-miR-4290_st | 0.594214 | 1.5 | 0.103205 | 0 |
| hsa-miR-200b-star_st | −1.06694 | 2.1 | 0.013656 | 1 |
| hsa-miR-4292_st | 0.532987 | 1.4 | 0.062882 | 0 |
| hsa-miR-4293_st | 0.635439 | 1.6 | 0.082819 | 0 |
| hsa-miR-1246_st | −1.0963 | 2.1 | 0.02776 | 1 |
| hsa-miR-4295_st | 0.08526 | 1.1 | 0.747239 | 0 |
| hsa-miR-4296_st | 0.527848 | 1.4 | 0.086917 | 0 |
| hsa-miR-4297_st | 0.734495 | 1.7 | 0.277893 | 0 |
| hsa-miR-4298_st | 0.359887 | 1.3 | 0.087727 | 0 |
| hsa-miR-218_st | −1.09644 | 2.1 | 0.043681 | 1 |
| hsa-miR-4286_st | −1.11281 | 2.2 | 0.003879 | 1 |
| hsa-miR-4301_st | −0.61789 | 1.5 | 0.059833 | 0 |
| hsa-miR-4302_st | 0.250463 | 1.2 | 0.512832 | 0 |
| hsa-miR-365_st | −1.11673 | 2.2 | 0.001388 | 1 |
| hsa-miR-4304_st | 0.631283 | 1.5 | 0.15302 | 0 |
| hsa-miR-4305_st | −0.23412 | 1.2 | 0.176014 | 0 |
| hsa-miR-4306_st | 0.066563 | 1.0 | 0.696751 | 0 |
| hsa-miR-26a_st | −1.1466 | 2.2 | 0.012738 | 1 |
| hsa-miR-4308_st | −0.23737 | 1.2 | 0.293694 | 0 |
| hsa-miR-4309_st | −0.20677 | 1.2 | 0.06645 | 0 |
| hsa-miR-186_st | −1.15647 | 2.2 | 0.012007 | 1 |
| hsa-miR-4310_st | 0.536411 | 1.5 | 0.079085 | 0 |
| hsa-miR-4311_st | 0.387188 | 1.3 | 0.201073 | 0 |
| hsa-miR-4312_st | 0.545958 | 1.5 | 0.187312 | 0 |
| hsa-miR-4313_st | −0.34859 | 1.3 | 0.199382 | 0 |
| hsa-miR-4314_st | 0.920085 | 1.9 | 0.05435 | 0 |
| hsa-miR-4315_st | 0.545556 | 1.5 | 0.141582 | 0 |
| hsa-miR-149_st | −1.16032 | 2.2 | 0.024674 | 1 |
| hsa-miR-106b-star_st | −1.16247 | 2.2 | 0.016346 | 1 |
| hsa-miR-4318_st | 0.40804 | 1.3 | 0.101943 | 0 |
| hsa-miR-4319_st | 0.376405 | 1.3 | 0.162053 | 0 |
| hsa-miR-29b-2-star_st | −1.18536 | 2.3 | 0.002818 | 1 |
| hsa-miR-432_st | 0.243124 | 1.2 | 0.498705 | 0 |
| hsa-miR-4320_st | −0.07682 | 1.1 | 0.775095 | 0 |
| hsa-miR-522_st | −0.24478 | 1.2 | 0.236455 | 0 |
| hsa-miR-4322_st | 0.365759 | 1.3 | 0.125926 | 0 |
| hsa-miR-4323_st | 0.428463 | 1.3 | 0.197673 | 0 |
| hsa-miR-93-star_st | −1.18842 | 2.3 | 0.000519 | 1 |
| hsa-miR-4325_st | −0.15717 | 1.1 | 0.677045 | 0 |
| hsa-miR-4326_st | 0.021366 | 1.0 | 0.898117 | 0 |
| hsa-miR-874_st | 0.472868 | 1.4 | 0.080117 | 0 |
| hsa-miR-4328_st | −0.06311 | 1.0 | 0.817515 | 0 |
| hsa-let-7e-star_st | −1.20176 | 2.3 | 0.000898 | 1 |
| hsa-miR-720_st | −0.61139 | 1.5 | 0.079327 | 0 |
| hsa-miR-433_st | 0.478322 | 1.4 | 0.237514 | 0 |
| hsa-miR-4330_st | 0.738696 | 1.7 | 0.186338 | 0 |
| hsa-miR-448_st | −0.23906 | 1.2 | 0.626683 | 0 |
| hsa-miR-449a_st | −0.23688 | 1.2 | 0.273276 | 0 |
| hsa-miR-449b_st | 0.255343 | 1.2 | 0.384548 | 0 |
| hsa-miR-1975_st | −0.59647 | 1.5 | 0.068224 | 0 |
| hsa-miR-449c_st | 0.229738 | 1.2 | 0.578924 | 0 |
| hsa-miR-449c-star_st | 0.339858 | 1.3 | 0.225351 | 0 |
| hsa-miR-1_st | −1.21267 | 2.3 | 0.017395 | 1 |
| hsa-miR-450b-3p_st | −0.2953 | 1.2 | 0.205481 | 0 |
| hsa-miR-450b-5p_st | −0.28982 | 1.2 | 0.31888 | 0 |
| hsa-miR-451_st | −0.03731 | 1.0 | 0.946044 | 0 |
| hsa-miR-452_st | 0.291868 | 1.2 | 0.395208 | 0 |
| hsa-miR-452-star_st | −0.1084 | 1.1 | 0.487922 | 0 |
| hsa-miR-548b-5p_st | 0.293886 | 1.2 | 0.056737 | 0 |
| hsa-miR-454-star_st | 0.275892 | 1.2 | 0.152569 | 0 |
| hsa-miR-455-3p_st | −0.74169 | 1.7 | 0.175044 | 0 |
| hsa-miR-455-5p_st | −0.46334 | 1.4 | 0.23172 | 0 |
| hsa-miR-466_st | −0.0117 | 1.0 | 0.928298 | 0 |
| hsa-miR-483-3p_st | −0.17839 | 1.1 | 0.586027 | 0 |
| hsa-miR-17_st | −1.24124 | 2.4 | 0.022884 | 1 |
| hsa-miR-320d_st | −1.24305 | 2.4 | 0.016573 | 1 |
| hsa-miR-485-3p_st | 0.265894 | 1.2 | 0.573541 | 0 |
| hsa-miR-485-5p_st | 0.567209 | 1.5 | 0.13375 | 0 |
| hsa-miR-486-3p_st | 0.324086 | 1.3 | 0.181653 | 0 |
| hsa-miR-486-5p_st | −0.23003 | 1.2 | 0.661842 | 0 |
| hsa-miR-644_st | 0.080448 | 1.1 | 0.793768 | 0 |
| hsa-miR-3152_st | 0.881141 | 1.8 | 0.134634 | 0 |
| hsa-miR-488_st | −0.01991 | 1.0 | 0.920397 | 0 |
| hsa-miR-488-star_st | 0.069247 | 1.0 | 0.766997 | 0 |
| hsa-miR-450a_st | −1.24402 | 2.4 | 0.000162 | 1 |
| hsa-miR-20a_st | −1.24779 | 2.4 | 0.043716 | 1 |
| hsa-miR-484_st | −1.29519 | 2.5 | 0.024706 | 1 |
| hsa-miR-491-3p_st | −0.13408 | 1.1 | 0.440417 | 0 |
| hsa-miR-491-5p_st | 0.041577 | 1.0 | 0.846465 | 0 |
| hsa-miR-197_st | −1.30773 | 2.5 | 0.000409 | 1 |
| hsa-miR-493_st | −0.04313 | 1.0 | 0.893703 | 0 |
| hsa-miR-493-star_st | 0.068753 | 1.0 | 0.867647 | 0 |
| hsa-miR-494_st | 0.345122 | 1.3 | 0.257661 | 0 |
| hsa-miR-495_st | −0.28479 | 1.2 | 0.518507 | 0 |
| hsa-miR-496_st | −0.38173 | 1.3 | 0.192888 | 0 |
| hsa-miR-497_st | −0.49443 | 1.4 | 0.417865 | 0 |
| hsa-miR-497-star_st | −0.03322 | 1.0 | 0.874365 | 0 |
| hsa-miR-498_st | 0.471247 | 1.4 | 0.099652 | 0 |
| hsa-miR-499-3p_st | −0.1898 | 1.1 | 0.217676 | 0 |
| hsa-miR-24-1-star_st | −1.31849 | 2.5 | 0.005603 | 1 |
| hsa-miR-340_st | −1.33011 | 2.5 | 0.00583 | 1 |
| hsa-miR-500b_st | −0.16416 | 1.1 | 0.642732 | 0 |
| hsa-miR-500-star_st | 0.157403 | 1.1 | 0.271468 | 0 |
| hsa-miR-501-3p_st | 0.25968 | 1.2 | 0.266636 | 0 |
| hsa-miR-501-5p_st | −0.07301 | 1.1 | 0.492835 | 0 |
| hsa-miR-502-3p_st | 0.119274 | 1.1 | 0.339925 | 0 |
| hsa-miR-502-5p_st | 0.268249 | 1.2 | 0.330544 | 0 |
| hsa-miR-503_st | 0.000161 | 1.0 | 0.999156 | 0 |
| hsa-miR-192_st | −1.33799 | 2.5 | 0.026824 | 1 |
| hsa-let-7g_st | −1.34037 | 2.5 | 0.031696 | 1 |
| hsa-miR-505-star_st | 0.20394 | 1.2 | 0.319073 | 0 |
| hsa-miR-506_st | −0.27491 | 1.2 | 0.205414 | 0 |
| hsa-miR-507_st | −0.19228 | 1.1 | 0.316814 | 0 |
| hsa-miR-508-3p_st | −0.2872 | 1.2 | 0.181928 | 0 |
| hsa-miR-508-5p_st | 0.370544 | 1.3 | 0.16899 | 0 |
| hsa-miR-509-3-5p_st | 0.77745 | 1.7 | 0.106877 | 0 |
| hsa-miR-509-3p_st | 0.309421 | 1.2 | 0.38079 | 0 |
| hsa-miR-106a_st | −1.37191 | 2.6 | 0.02611 | 1 |
| hsa-miR-106b_st | −1.38069 | 2.6 | 0.009763 | 1 |
| hsa-miR-511_st | 0.122845 | 1.1 | 0.614312 | 0 |
| hsa-miR-512-3p_st | 0.390853 | 1.3 | 0.105227 | 0 |
| hsa-miR-512-5p_st | 0.660443 | 1.6 | 0.159525 | 0 |
| hsa-miR-513a-3p_st | −0.20092 | 1.1 | 0.5732 | 0 |
| hsa-miR-23a_st | −1.40296 | 2.6 | 0.016684 | 1 |
| hsa-miR-25_st | −1.41356 | 2.7 | 0.029964 | 1 |
| hsa-miR-598_st | −1.41999 | 2.7 | 0.004541 | 1 |
| hsa-miR-1260_st | −1.42155 | 2.7 | 0.025045 | 1 |
| hsa-miR-514b-3p_st | 0.079916 | 1.1 | 0.792966 | 0 |
| hsa-miR-514b-5p_st | 0.451744 | 1.4 | 0.056935 | 0 |
| hsa-miR-515-3p_st | 0.257161 | 1.2 | 0.372246 | 0 |
| hsa-miR-515-5p_st | −0.14137 | 1.1 | 0.551557 | 0 |
| hsa-miR-516a-3p_st | −0.3671 | 1.3 | 0.20403 | 0 |
| hsa-miR-27a_st | −1.43065 | 2.7 | 0.032296 | 1 |
| hsa-miR-100_st | −1.466 | 2.8 | 0.015928 | 1 |
| hsa-miR-101_st | −1.46742 | 2.8 | 0.047468 | 1 |
| hsa-miR-517a_st | −0.11374 | 1.1 | 0.729152 | 0 |
| hsa-miR-517b_st | −0.22637 | 1.2 | 0.296381 | 0 |
| hsa-miR-517c_st | −0.22077 | 1.2 | 0.390675 | 0 |
| hsa-miR-517-star_st | 0.286517 | 1.2 | 0.180976 | 0 |
| hsa-miR-518a-3p_st | 0.122831 | 1.1 | 0.509466 | 0 |
| hsa-miR-518a-5p_st | 0.450068 | 1.4 | 0.336769 | 0 |
| hsa-miR-518b_st | −0.23318 | 1.2 | 0.311518 | 0 |
| hsa-miR-518c_st | −0.33735 | 1.3 | 0.228688 | 0 |
| hsa-miR-301a_st | −1.46999 | 2.8 | 0.009301 | 1 |
| hsa-miR-518d-3p_st | −0.10291 | 1.1 | 0.518346 | 0 |
| hsa-miR-518d-5p_st | 0.229352 | 1.2 | 0.416537 | 0 |
| hsa-miR-518e_st | −0.35396 | 1.3 | 0.331555 | 0 |
| hsa-let-7f_st | −1.47955 | 2.8 | 0.023454 | 1 |
| hsa-miR-518f_st | −0.10237 | 1.1 | 0.667527 | 0 |
| hsa-miR-1826_st | −0.45843 | 1.4 | 0.151401 | 0 |
| hsa-miR-519a_st | −0.11837 | 1.1 | 0.694239 | 0 |
| hsa-miR-519a-star_st | 0.441549 | 1.4 | 0.261878 | 0 |
| hsa-miR-519b-3p_st | −0.47802 | 1.4 | 0.115601 | 0 |
| hsa-miR-1283_st | 0.019298 | 1.0 | 0.917184 | 0 |
| hsa-miR-519c-3p_st | −0.20606 | 1.2 | 0.37948 | 0 |
| hsa-miR-519c-5p_st | 0.450758 | 1.4 | 0.086822 | 0 |
| hsa-miR-519d_st | −0.15044 | 1.1 | 0.548921 | 0 |
| hsa-miR-519e_st | −0.57309 | 1.5 | 0.073961 | 0 |
| hsa-miR-519e-star_st | 0.818031 | 1.8 | 0.084783 | 0 |
| hsa-miR-520a-3p_st | 0.30561 | 1.2 | 0.167727 | 0 |
| hsa-miR-1908_st | −0.46528 | 1.4 | 0.146264 | 0 |
| hsa-miR-520b_st | −0.0826 | 1.1 | 0.785643 | 0 |
| hsa-miR-520c-3p_st | −0.11881 | 1.1 | 0.719481 | 0 |
| hsa-miR-520c-5p_st | 0.313653 | 1.2 | 0.352911 | 0 |
| hsa-miR-181a-star_st | −1.48692 | 2.8 | 0.014503 | 1 |
| hsa-miR-21_st | −1.49665 | 2.8 | 0.013004 | 1 |
| hsa-miR-520e_st | −0.00382 | 1.0 | 0.987061 | 0 |
| hsa-miR-520f_st | −0.29484 | 1.2 | 0.260527 | 0 |
| hsa-miR-520g_st | 0.025867 | 1.0 | 0.863878 | 0 |
| hsa-miR-520h_st | −0.23061 | 1.2 | 0.465788 | 0 |
| hsa-miR-521_st | −0.4412 | 1.4 | 0.090071 | 0 |
| hsa-miR-302a-star_st | 0.183684 | 1.1 | 0.370531 | 0 |
| hsa-miR-886-3p_st | −1.49782 | 2.8 | 0.033148 | 1 |
| hsa-miR-523_st | −0.23984 | 1.2 | 0.483016 | 0 |
| hsa-miR-29a_st | −1.5044 | 2.8 | 0.015461 | 1 |
| hsa-miR-524-3p_st | 0.170023 | 1.1 | 0.211849 | 0 |
| hsa-miR-142-3p_st | 0.255482 | 1.2 | 0.622115 | 0 |
| hsa-miR-525-3p_st | −0.12933 | 1.1 | 0.625719 | 0 |
| hsa-miR-525-5p_st | −0.07056 | 1.1 | 0.735299 | 0 |
| hsa-miR-762_st | −0.43808 | 1.4 | 0.165812 | 0 |
| hsa-miR-423-3p_st | −1.50794 | 2.8 | 0.000424 | 1 |
| hsa-miR-449b-star_st | 0.559689 | 1.5 | 0.178184 | 0 |
| hsa-miR-527_st | 0.47098 | 1.4 | 0.137194 | 0 |
| hsa-miR-532-3p_st | −1.01366 | 2.0 | 0.067465 | 0 |
| hsa-miR-532-5p_st | −0.63302 | 1.6 | 0.147815 | 0 |
| hsa-miR-539_st | 0.126512 | 1.1 | 0.719238 | 0 |
| hsa-let-7a_st | −1.54323 | 2.9 | 0.018056 | 1 |
| hsa-miR-541-star_st | 0.21097 | 1.2 | 0.193034 | 0 |
| hsa-miR-542-3p_st | 0.152322 | 1.1 | 0.591227 | 0 |
| hsa-miR-542-5p_st | −0.6952 | 1.6 | 0.16266 | 0 |
| hsa-miR-543_st | −0.42727 | 1.3 | 0.074664 | 0 |
| hsa-miR-544_st | −0.18347 | 1.1 | 0.623766 | 0 |
| hsa-miR-544b_st | 0.127209 | 1.1 | 0.603508 | 0 |
| hsa-miR-545_st | −0.26396 | 1.2 | 0.336005 | 0 |
| hsa-miR-545-star_st | −0.52642 | 1.4 | 0.128244 | 0 |
| hsa-miR-548a-3p_st | −0.38127 | 1.3 | 0.366102 | 0 |
| hsa-miR-2861_st | −0.45367 | 1.4 | 0.154287 | 0 |
| hsa-miR-31_st | −1.5512 | 2.9 | 0.018739 | 1 |
| hsa-miR-367_st | −0.18755 | 1.1 | 0.478768 | 0 |
| hsa-miR-548c-3p_st | −0.59016 | 1.5 | 0.249024 | 0 |
| hsa-miR-548c-5p_st | 0.165415 | 1.1 | 0.824566 | 0 |
| hsa-miR-548d-3p_st | −0.15575 | 1.1 | 0.462901 | 0 |
| hsa-miR-548d-5p_st | −0.17104 | 1.1 | 0.702449 | 0 |
| hsa-miR-548e_st | −0.0759 | 1.1 | 0.824529 | 0 |
| hsa-miR-548f_st | −0.47056 | 1.4 | 0.174292 | 0 |
| hsa-miR-548g_st | −0.56438 | 1.5 | 0.245965 | 0 |
| hsa-miR-548h_st | −0.3003 | 1.2 | 0.177204 | 0 |
| hsa-miR-548i_st | 0.078643 | 1.1 | 0.734091 | 0 |
| hsa-miR-548j_st | 0.25418 | 1.2 | 0.318076 | 0 |
| hsa-miR-548k_st | −0.21967 | 1.2 | 0.084338 | 0 |
| hsa-miR-526b-star_st | −0.12919 | 1.1 | 0.707069 | 0 |
| hsa-miR-548m_st | 0.066115 | 1.0 | 0.792698 | 0 |
| hsa-miR-548n_st | −0.17766 | 1.1 | 0.567752 | 0 |
| hsa-miR-548o_st | −0.25402 | 1.2 | 0.360821 | 0 |
| hsa-miR-548p_st | −0.17622 | 1.1 | 0.446389 | 0 |
| hsa-miR-548q_st | 0.71986 | 1.6 | 0.067832 | 0 |
| hsa-miR-548s_st | −0.06254 | 1.0 | 0.708593 | 0 |
| hsa-miR-548t_st | 0.191443 | 1.1 | 0.496084 | 0 |
| hsa-miR-548u_st | −0.6602 | 1.6 | 0.106665 | 0 |
| hsa-miR-638_st | −0.50391 | 1.4 | 0.122174 | 0 |
| hsa-miR-548w_st | −0.07509 | 1.1 | 0.874394 | 0 |
| hsa-miR-3119_st | 0.023444 | 1.0 | 0.945048 | 0 |
| hsa-miR-549_st | 0.008477 | 1.0 | 0.973793 | 0 |
| hsa-miR-205_st | −1.55979 | 2.9 | 0.037086 | 1 |
| hsa-miR-550-star_st | −0.08505 | 1.1 | 0.623248 | 0 |
| hsa-miR-551a_st | 0.25166 | 1.2 | 0.39002 | 0 |
| hsa-miR-93_st | −1.56661 | 3.0 | 0.025987 | 1 |
| hsa-miR-551b-star_st | −0.25958 | 1.2 | 0.150179 | 0 |
| hsa-let-7d-star_st | −1.57161 | 3.0 | 1E−06 | 1 |
| hsa-miR-553_st | −0.35993 | 1.3 | 0.190737 | 0 |
| hsa-miR-554_st | 0.096033 | 1.1 | 0.7478 | 0 |
| hsa-miR-555_st | −0.06063 | 1.0 | 0.724188 | 0 |
| hsa-miR-556-3p_st | −0.06018 | 1.0 | 0.827136 | 0 |
| hsa-miR-556-5p_st | −0.59567 | 1.5 | 0.13322 | 0 |
| hsa-miR-557_st | 0.440682 | 1.4 | 0.085371 | 0 |
| hsa-miR-558_st | −0.38111 | 1.3 | 0.376246 | 0 |
| hsa-miR-559_st | −0.24743 | 1.2 | 0.451607 | 0 |
| hsa-miR-561_st | −0.37003 | 1.3 | 0.2507 | 0 |
| hsa-miR-562_st | −0.42219 | 1.3 | 0.171043 | 0 |
| hsa-miR-563_st | −0.07291 | 1.1 | 0.646014 | 0 |
| hsa-miR-361-5p_st | −1.5803 | 3.0 | 0.004489 | 1 |
| hsa-miR-566_st | 0.24973 | 1.2 | 0.206058 | 0 |
| hsa-miR-567_st | 0.210488 | 1.2 | 0.288931 | 0 |
| hsa-miR-568_st | −0.43354 | 1.4 | 0.155504 | 0 |
| hsa-miR-29c_st | −1.61118 | 3.1 | 0.008279 | 1 |
| hsa-miR-570_st | −0.15191 | 1.1 | 0.656083 | 0 |
| hsa-miR-571_st | −0.25703 | 1.2 | 0.232749 | 0 |
| hsa-miR-572_st | 0.30286 | 1.2 | 0.275827 | 0 |
| hsa-miR-573_st | −0.12504 | 1.1 | 0.60286 | 0 |
| hsa-miR-132_st | −1.61895 | 3.1 | 0.006005 | 1 |
| hsa-miR-574-5p_st | 0.102743 | 1.1 | 0.37019 | 0 |
| hsa-let-7d_st | −1.62762 | 3.1 | 0.017634 | 1 |
| hsa-miR-576-3p_st | 0.006569 | 1.0 | 0.976898 | 0 |
| hsa-miR-576-5p_st | 0.324087 | 1.3 | 0.303538 | 0 |
| hsa-miR-577_st | −0.05216 | 1.0 | 0.863778 | 0 |
| hsa-miR-578_st | −0.41059 | 1.3 | 0.113335 | 0 |
| hsa-miR-579_st | −0.30904 | 1.2 | 0.111718 | 0 |
| hsa-miR-580_st | −0.36563 | 1.3 | 0.289385 | 0 |
| hsa-miR-191_st | −1.63421 | 3.1 | 0.020089 | 1 |
| hsa-miR-582-3p_st | −0.01156 | 1.0 | 0.967935 | 0 |
| hsa-miR-582-5p_st | 0.751847 | 1.7 | 0.242341 | 0 |
| hsa-miR-1915_st | −0.5001 | 1.4 | 0.124304 | 0 |
| hsa-let-7c_st | −1.66897 | 3.2 | 0.012062 | 1 |
| hsa-miR-585_st | −0.05951 | 1.0 | 0.815908 | 0 |
| hsa-miR-586_st | −0.12226 | 1.1 | 0.630922 | 0 |
| hsa-miR-324-5p_st | −1.67397 | 3.2 | 0.000385 | 1 |
| hsa-miR-588_st | 0.266646 | 1.2 | 0.265895 | 0 |
| hsa-miR-589_st | 0.051192 | 1.0 | 0.85737 | 0 |
| hsa-miR-589-star_st | 0.12791 | 1.1 | 0.576093 | 0 |
| hsa-miR-188-5p_st | 0.414976 | 1.3 | 0.223638 | 0 |
| hsa-miR-103_st | −1.6799 | 3.2 | 0.0199 | 1 |
| hsa-miR-591_st | 0.246684 | 1.2 | 0.479477 | 0 |
| hsa-miR-592_st | −0.35004 | 1.3 | 0.126418 | 0 |
| hsa-miR-593_st | 0.279291 | 1.2 | 0.366004 | 0 |
| hsa-miR-23b_st | −1.67999 | 3.2 | 0.009157 | 1 |
| hsa-miR-595_st | 0.221336 | 1.2 | 0.407849 | 0 |
| hsa-miR-99a_st | −1.7069 | 3.3 | 0.015518 | 1 |
| hsa-miR-15b_st | −1.7135 | 3.3 | 0.007437 | 1 |
| hsa-miR-135b_st | −1.72432 | 3.3 | 0.00367 | 1 |
| hsa-miR-599_st | −0.39735 | 1.3 | 0.202803 | 0 |
| hsa-miR-600_st | −0.42431 | 1.3 | 0.262063 | 0 |
| hsa-miR-107_st | −1.7247 | 3.3 | 0.017993 | 1 |
| hsa-miR-602_st | 0.244492 | 1.2 | 0.244548 | 0 |
| hsa-miR-603_st | −0.35019 | 1.3 | 0.287249 | 0 |
| hsa-miR-3065-5p_st | −1.7278 | 3.3 | 2E−05 | 1 |
| hsa-miR-605_st | 0.654207 | 1.6 | 0.241571 | 0 |
| hsa-miR-24_st | −1.73165 | 3.3 | 0.012912 | 1 |
| hsa-miR-607_st | −0.38094 | 1.3 | 0.230152 | 0 |
| hsa-miR-608_st | 0.399753 | 1.3 | 0.214128 | 0 |
| hsa-miR-609_st | −0.01148 | 1.0 | 0.971304 | 0 |
| hsa-miR-3196_st | −0.51976 | 1.4 | 0.113395 | 0 |
| hsa-miR-26b_st | −1.74563 | 3.4 | 0.010462 | 1 |
| hsa-miR-28-5p_st | −1.81666 | 3.5 | 0.015131 | 1 |
| hsa-miR-613_st | 0.463317 | 1.4 | 0.168306 | 0 |
| hsa-miR-338-3p_st | −1.83368 | 3.6 | 0.000214 | 1 |
| hsa-miR-615-3p_st | 0.518109 | 1.4 | 0.19134 | 0 |
| hsa-miR-454_st | −1.84034 | 3.6 | 0.004066 | 1 |
| hsa-miR-616_st | 0.26552 | 1.2 | 0.445685 | 0 |
| hsa-miR-151-5p_st | −1.8621 | 3.6 | 0.016985 | 1 |
| hsa-miR-617_st | 0.69404 | 1.6 | 0.261134 | 0 |
| hsa-miR-128_st | −1.88819 | 3.7 | 0.018707 | 1 |
| hsa-miR-619_st | 0.153134 | 1.1 | 0.362526 | 0 |
| hsa-miR-620_st | 0.109661 | 1.1 | 0.564687 | 0 |
| hsa-miR-621_st | −0.45683 | 1.4 | 0.145843 | 0 |
| hsa-let-7b_st | −1.93383 | 3.8 | 0.004097 | 1 |
| hsa-miR-623_st | 0.312316 | 1.2 | 0.305791 | 0 |
| hsa-miR-624_st | −0.38213 | 1.3 | 0.118097 | 0 |
| hsa-miR-624-star_st | −0.36289 | 1.3 | 0.171384 | 0 |
| hsa-miR-183_st | −1.93409 | 3.8 | 0.006078 | 1 |
| hsa-miR-30b_st | −1.97702 | 3.9 | 0.018453 | 1 |
| hsa-miR-626_st | −0.10175 | 1.1 | 0.547901 | 0 |
| hsa-miR-627_st | −0.35975 | 1.3 | 0.21916 | 0 |
| hsa-miR-628-3p_st | 0.714063 | 1.6 | 0.052801 | 0 |
| hsa-miR-31-star_st | −1.97746 | 3.9 | 0.025758 | 1 |
| hsa-miR-218-1-star_st | 0.171178 | 1.1 | 0.317876 | 0 |
| hsa-miR-629-star_st | −0.12576 | 1.1 | 0.678675 | 0 |
| hsa-miR-630_st | 0.956088 | 1.9 | 0.093918 | 0 |
| hsa-miR-631_st | 0.751588 | 1.7 | 0.110098 | 0 |
| hsa-miR-632_st | 0.169891 | 1.1 | 0.666667 | 0 |
| hsa-miR-633_st | −0.23553 | 1.2 | 0.253977 | 0 |
| hsa-miR-634_st | 0.363455 | 1.3 | 0.294704 | 0 |
| hsa-miR-635_st | 0.413661 | 1.3 | 0.18374 | 0 |
| hsa-miR-203_st | −2.00197 | 4.0 | 0.008637 | 1 |
| hsa-miR-331-3p_st | −2.00221 | 4.0 | 0.017173 | 1 |
| hsa-miR-125b_st | −2.0088 | 4.0 | 0.001535 | 1 |
| hsa-let-7i_st | −2.01069 | 4.0 | 0.010629 | 1 |
| hsa-miR-640_st | 0.36472 | 1.3 | 0.367639 | 0 |
| hsa-miR-641_st | 0.112934 | 1.1 | 0.689435 | 0 |
| hsa-miR-642_st | −0.10501 | 1.1 | 0.495134 | 0 |
| hsa-miR-29b_st | −2.03746 | 4.1 | 0.003653 | 1 |
| hsa-miR-122_st | 0.260995 | 1.2 | 0.404904 | 0 |
| hsa-miR-645_st | 0.442712 | 1.4 | 0.222791 | 0 |
| hsa-miR-646_st | −0.10513 | 1.1 | 0.592753 | 0 |
| hsa-miR-647_st | 0.116209 | 1.1 | 0.45229 | 0 |
| hsa-miR-648_st | 0.517924 | 1.4 | 0.098509 | 0 |
| hsa-miR-649_st | 0.427329 | 1.3 | 0.242068 | 0 |
| hsa-miR-182_st | −2.05592 | 4.2 | 0.039156 | 1 |
| hsa-miR-651_st | 0.136891 | 1.1 | 0.211866 | 0 |
| hsa-miR-652_st | −0.29319 | 1.2 | 0.39282 | 0 |
| hsa-miR-653_st | −0.17913 | 1.1 | 0.502094 | 0 |
| hsa-miR-654-3p_st | −0.09408 | 1.1 | 0.806228 | 0 |
| hsa-miR-374b_st | −2.06484 | 4.2 | 0.013714 | 1 |
| hsa-miR-130a_st | −2.07106 | 4.2 | 0.001563 | 1 |
| hsa-miR-656_st | −0.18179 | 1.1 | 0.453615 | 0 |
| hsa-miR-657_st | 0.524405 | 1.4 | 0.078783 | 0 |
| hsa-miR-658_st | 0.396407 | 1.3 | 0.053112 | 0 |
| hsa-miR-99b_st | −2.07354 | 4.2 | 0.003615 | 1 |
| hsa-miR-660_st | −1.00935 | 2.0 | 0.177414 | 0 |
| hsa-miR-661_st | 0.556038 | 1.5 | 0.211682 | 0 |
| hsa-miR-30e-star_st | −2.07466 | 4.2 | 0.02214 | 1 |
| hsa-miR-663_st | −0.19822 | 1.1 | 0.492136 | 0 |
| hsa-miR-663b_st | 0.683826 | 1.6 | 0.059632 | 0 |
| hsa-miR-664_st | −0.67316 | 1.6 | 0.315716 | 0 |
| hsa-miR-664-star_st | 0.238579 | 1.2 | 0.646885 | 0 |
| hsa-miR-30d_st | −2.07642 | 4.2 | 0.016353 | 1 |
| hsa-miR-668_st | 0.58339 | 1.5 | 0.221596 | 0 |
| hsa-miR-670_st | 0.401209 | 1.3 | 0.371453 | 0 |
| hsa-miR-671-3p_st | 0.278112 | 1.2 | 0.086159 | 0 |
| hsa-miR-671-5p_st | 0.503639 | 1.4 | 0.066593 | 0 |
| hsa-miR-30a-star_st | −2.08511 | 4.2 | 0.031655 | 1 |
| hsa-miR-675-star_st | −0.04357 | 1.0 | 0.766069 | 0 |
| hsa-miR-148b_st | −2.09371 | 4.3 | 0.030988 | 1 |
| hsa-miR-708_st | −0.54092 | 1.5 | 0.179872 | 0 |
| hsa-miR-708-star_st | −0.13177 | 1.1 | 0.651749 | 0 |
| hsa-miR-221-star_st | −2.10599 | 4.3 | 0.001863 | 1 |
| hsa-let-7e_st | −2.12879 | 4.4 | 0.01363 | 1 |
| hsa-miR-7-1-star_st | −0.59785 | 1.5 | 0.198372 | 0 |
| hsa-miR-27b_st | −2.1404 | 4.4 | 0.020858 | 1 |
| hsa-miR-34a_st | −2.16416 | 4.5 | 0.016747 | 1 |
| hsa-miR-744_st | 0.385006 | 1.3 | 0.068587 | 0 |
| hsa-miR-744-star_st | −0.05725 | 1.0 | 0.788584 | 0 |
| hsa-miR-92b_st | −2.18683 | 4.6 | 9.76E−05 | 1 |
| hsa-miR-759_st | −0.24225 | 1.2 | 0.329115 | 0 |
| hsa-miR-760_st | 0.37276 | 1.3 | 0.074669 | 0 |
| hsa-miR-92a_st | −2.2569 | 4.8 | 0.001851 | 1 |
| hsa-miR-335_st | −2.27502 | 4.8 | 0.010623 | 1 |
| hsa-miR-96_st | −2.27634 | 4.8 | 0.038554 | 1 |
| hsa-miR-95_st | −2.31054 | 5.0 | 0.007726 | 1 |
| hsa-miR-766_st | 0.932585 | 1.9 | 0.149437 | 0 |
| hsa-miR-767-3p_st | −0.01512 | 1.0 | 0.946471 | 0 |
| hsa-miR-767-5p_st | −0.00328 | 1.0 | 0.990581 | 0 |
| hsa-miR-98_st | −2.31096 | 5.0 | 0.015734 | 1 |
| hsa-miR-769-5p_st | −0.17583 | 1.1 | 0.480561 | 0 |
| hsa-miR-770-5p_st | 0.212152 | 1.2 | 0.561233 | 0 |
| hsa-miR-802_st | −0.18983 | 1.1 | 0.471489 | 0 |
| hsa-miR-34b-star_st | −2.33393 | 5.0 | 0.013135 | 1 |
| hsa-miR-581_st | 0.038637 | 1.0 | 0.929187 | 0 |
| hsa-miR-181d_st | −2.38781 | 5.2 | 5.43E−05 | 1 |
| hsa-miR-875-5p_st | −0.24512 | 1.2 | 0.437978 | 0 |
| hsa-miR-876-3p_st | 0.301243 | 1.2 | 0.272646 | 0 |
| hsa-miR-876-5p_st | −0.34164 | 1.3 | 0.218447 | 0 |
| hsa-miR-151-3p_st | −2.39852 | 5.3 | 0.010408 | 1 |
| hsa-miR-877-star_st | 0.85547 | 1.8 | 0.061317 | 0 |
| hsa-miR-885-3p_st | 0.184839 | 1.1 | 0.377934 | 0 |
| hsa-miR-200c_st | −2.41315 | 5.3 | 0.007772 | 1 |
| hsa-miR-429_st | −2.49862 | 5.7 | 0.000779 | 1 |
| hsa-miR-886-5p_st | −0.31579 | 1.2 | 0.086841 | 0 |
| hsa-miR-224_st | −2.57625 | 6.0 | 6.34E−07 | 1 |
| hsa-miR-888_st | 0.138689 | 1.1 | 0.585938 | 0 |
| hsa-miR-4321_st | 0.775601 | 1.7 | 0.052804 | 0 |
| hsa-miR-889_st | 0.118195 | 1.1 | 0.560606 | 0 |
| hsa-miR-181b_st | −2.58165 | 6.0 | 4.34E−07 | 1 |
| hsa-miR-891a_st | 0.218514 | 1.2 | 0.431066 | 0 |
| hsa-miR-891b_st | −0.55826 | 1.5 | 0.078082 | 0 |
| hsa-miR-892a_st | 0.344266 | 1.3 | 0.472797 | 0 |
| hsa-miR-892b_st | 0.226274 | 1.2 | 0.458406 | 0 |
| hsa-miR-9_st | −0.09564 | 1.1 | 0.811743 | 0 |
| hsa-miR-222_st | −2.59688 | 6.0 | 0.000732 | 1 |
| hsa-miR-141_st | −2.59852 | 6.1 | 0.002638 | 1 |
| hsa-miR-221_st | −2.70452 | 6.5 | 0.001066 | 1 |
| hsa-miR-924_st | 0.07618 | 1.1 | 0.622376 | 0 |
| hsa-miR-181a_st | −2.74062 | 6.7 | 0.000111 | 1 |
| hsa-miR-181c_st | −2.77321 | 6.8 | 0.00094 | 1 |
| hsa-miR-4324_st | −2.7793 | 6.9 | 0.000579 | 1 |
| hsa-miR-125a-5p_st | −2.78675 | 6.9 | 0.000273 | 1 |
| hsa-miR-92b-star_st | 0.161865 | 1.1 | 0.441506 | 0 |
| hsa-miR-3195_st | 0.545451 | 1.5 | 0.105432 | 0 |
| hsa-miR-3137_st | 0.714678 | 1.6 | 0.13027 | 0 |
| hsa-miR-934_st | −0.10872 | 1.1 | 0.733275 | 0 |
| hsa-miR-935_st | −0.26142 | 1.2 | 0.554128 | 0 |
| hsa-miR-551b_st | −2.9308 | 7.6 | 2.75E−08 | 1 |
| hsa-miR-937_st | 0.433089 | 1.4 | 0.171528 | 0 |
| hsa-miR-938_st | 0.206369 | 1.2 | 0.57125 | 0 |
| hsa-miR-939_st | 0.428522 | 1.3 | 0.123188 | 0 |
| hsa-miR-7-2-star_st | −0.63671 | 1.6 | 0.202747 | 0 |
| hsa-miR-146b-5p_st | −3.07337 | 8.4 | 0.000759 | 1 |
| hsa-miR-941_st | −0.02729 | 1.0 | 0.920459 | 0 |
| hsa-miR-942_st | −0.15221 | 1.1 | 0.584159 | 0 |
| hsa-miR-200a_st | −3.20855 | 9.2 | 0.001744 | 1 |
| hsa-miR-944_st | −0.38554 | 1.3 | 0.210878 | 0 |
| hsa-miR-7_st | −0.95705 | 1.9 | 0.213978 | 0 |
| hsa-miR-375_st | −3.22767 | 9.4 | 5.75E−10 | 1 |
| hsa-miR-96-star_st | −0.07371 | 1.1 | 0.531711 | 0 |
| hsa-miR-424_st | −3.28988 | 9.8 | 0.001702 | 1 |
| hsa-miR-181a-2-star_st | −3.36316 | 10.3 | 0.001188 | 1 |
| hsa-miR-200b_st | −3.59322 | 12.1 | 0.004911 | 1 |
| hsa-miR-146b-3p_st | −3.7959 | 13.9 | 3.15E−08 | 1 |
| hsa-miR-99b-star_st | −0.20555 | 1.2 | 0.24171 | 0 |
| hsa-miR-9-star_st | 0.757846 | 1.7 | 0.054881 | 0 |

TABLE O
List of miRNAs

| | | | SEQ |
|---|---|---|---|
| | Alternate | | ID |
| miR Name | Name | Sequence | NO: |
| miR-375 | miR-375 | UUUGUUCGUUCGGCUCGCGUGA | 13 |
| miR-7 | miR-7-5p | UGGAAGACUAGUGAUUUUGUUGU | 2 |
| miR-15a | miR-15a-5p | UAGCAGCACAUAAUGGUUUGUG | 36 |
| miR-19b | miR49b-3p | UGUGCAAAUCCAUGCAAAACUGA | 90 |
| miR-21 | miR-21-5p | UAGCUUAUCAGACUGAUGUUGA | 22 |
| miR-29b | miR-29b-3p | UAGCUUAUCAGACUGAUGUUGA | 139 |
| miR-29b- | miR-29b-1-5p | GCUGGUUUCAUAUGGUGGUUUAGA | 138 |
| 1* | | | |
| miR-31 | miR-31-5p | AGGCAAGAUGCUGGCAUAGCU | 18 |
| miR-31* | miR-31-3p | UGCUAUGCCAACAUAUUGCCAU | 41 |
| miR-34a | miR-34a-5p | UGGCAGUGUCUUAGCUGGUUGU | 23 |
| miR-34a* | miR-34a-3p | CAAUCAGCAAGUAUACUGCCCU | 42 |
| miR-34b* | miR-34b-5p | UAGGCAGUGUCAUUAGCUGAUUG | 43 |
| miR-96 | r6R-96-5p | UUUGLXACUAGCACAUUUUUGCU | 129 |
| miR-138- | miR-138-1-3p | GCUACUUCACAACACCAGGGCC | 24 |
| 1* | | | |
| miR-139- | miR-139-5p | UCUACAGUGCACGUGUCUCCAG | 25 |
| 5p | | | |
| miR-141 | miR-141-3p | UAACACUGUCUGGUAAAGAUGG | 26 |
| miR-146b- | miR-146b-3p | UGCCCUGUGGACUCAGUUCUGG | 4 |
| 3p | | | |
| miR-146b- | miR-14611-5p | UGAGAACUGAAUUCCAUAGGCU | 3 |
| 5p | | | |
| miR-151- | miR-151a-3p | CUAGACUGAAGCUCCUUGAGG | 388 |
| 3p | | | |
| miR-155 | miR-155-5p | UUAAUGCUAAUCGUGAUAGGGGU | 28 |
| miR-181a- | miR-181a-2-3p | ACCACUGACCGUUGACUGUACC | 178 |
| 2* | | | |
| miR-197 | miR-197-3p | UUCACCACCUUCUCCACCCAGC | 30 |
| miR-204 | miR-204-5p | UUCCCUUUGUCAUCCUAUGCCU | 192 |
| miR-221 | miR-221-3p | AGCUACAUUGUCUGCUGGGUUUC | 17 |
| miR-221* | miR-221-5p | ACCUGGcAUACAAUGUAGAUUU | 7 |
| miR-222 | miR-222-3p | AGCUACAVCUGGCUACUGGGU | 31 |
| miR-328 | miR-328 | CUGGCCCUCUCUGCCCUUCCGU | 379 |
| miR-424 | miR-424-5p | CAGCAGCAAUUCAUGUUUUGAA | 413 |
| miR-551b | miR-551b-3p | GCGACCCAUACUUGGUUUCAG | 54 |

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,384,261
U.S. Pat. No. 5,405,783
U.S. Pat. No. 5,412,087
U.S. Pat. No. 5,424,186
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent application Ser. No. 09/545,207
U.S. patent application Ser. No. 10/667,126
U.S. patent application Ser. No. 11/141,707
U.S. patent application Ser. No. 11/273,640
U.S. Patent Prov. Appn. Ser. No. 60/575,743
U.S. Patent Prov. Appn. Ser. No. 60/649,584
U.S. Patent Prov. Appn. Ser. No. 60/826,173
Akao et al., DNA Cell Biol., 26:311-320, 2007.
All et al., Ann. Trans. Res., 3:28-47, 2010.
Allen et al., Ann. Surg., 250:754-760, 2009.
Ambros, Cell, 107(7):823-826, 2001.
Beaucage, and Lyer, Tetrahedron, 48:2223-2311, 1992.
Brennecke et al., Cell, 113:25-36, 2003.
Calin et al., Proc. Natl. Acad. Sci. USA, 99:15524-15529, 2002.
Carrington et al. Science, 301(5631):336-338, 2003.
Chan et al., Anticancer Res., 28:907-911, 2008.
Chen et al., BMC Genomnics, 10:407, 2009.
Chou et al., Thyroid 20(5):489-493, 2010.
Cummins et al., In: IRT: Nucleosides and nucleosides, La Jolla Calif., 72, 1996.
Denli et al., Trends Biochem. Sci., 28:196, 2003.
Didenko, Biotechniques, 31(5):1106-16; 1118, 1120-I, 2001.
Dillhoff et al., J. Gastrointest. Surg., 12:2171-2176, 2008.
DiMagno, Clin. Gastroenterol. Hepatol., 5:797-798, 2007.
Doleshal et al., J. Mol. Diagn., 10:203-211, 2008.
Emptage et al., Neuron, 2001 January; 29(1):197-208, 2001.
EP 266,032
EP 373 203
EP 785 280
EP 799 897
Esquela-Kerscher and Slack, Nat Rev Cancer, 6(4):259-269, 2006.
Fodor et al., Science, 251:767-777, 1991.
Freelove and Walling, Am. Fat. Physician, 73(3):485-492, 2006.
Froehler et al., Nucleic Acids Res., 14(13):5399-5407, 1986:
Gillam et al., J. Biol. Chem., 253:2532, 1978.
Gillam et al., Nucleic Acids Res., 6:2973, 1979.
Giovannetti et al., Cancer Res., 70:4528-4538, 2010.
Gironella et al., Proc. Natl. Acad. Sci. USA, 104:16170-16175, 2007.
Gottardo et al., Urologic Oncol. Sem, Orig. Invest., 25:387-392, 2007.
Griffey e al., J Mass Spectrom, 32(3):305-13, 1997.
Habbe et al., Cancer Biol. Ther., 8:340-346, 2009.
He et al., Proc. Natl. Acad. Sci. USA, 102(52):19075-19080, 2005.
Hu et al. Int. J. Cancer, 128:132-143, 2011.
Iorio et al., Cancer Res., 65:7065-7070, 2005.
Itakura and Riggs, Science, 209:1401-1405, 1980.
Itakura et al., J. Biol. Chem., 250:4592, 1975.
Jemal et al., CA Cancer J Clin., 56(2):106-130, 2006.
Khalid et al., Clin. Gastroenterol. Hepatol., 3:967-973, 2005.
Khalid et al., Gastrointest. Endosc., 69:1095-1102, 2009.
Khorana, Science, 203, 614, 1979.
Kimura et al., Int. Pancreatol., 18:197-206, 1995.
Klostermeier and Millar, Biopolymers, 61(3):159-79, 2001-2002.
Kornberg and Baker, In: DNA Replication, 2d Ed., Freeman, San Francisco, 1992.
Lagos-Quintana et al., Science, 294(5543):853-858, 2001.
Lanza et al., Mol Cancer, 6:54, 2007.
Lau et al., Science, 294(5543):858-862, 2001.
Lee and Ambros, Science, 294(5543):862-864, 2001.
Lee et al., EMBO J. 21:4663-70, 2002.
Lu et al., Nature, 435(7043):834-838, 2005.
Matthaei and Maitra, In: Pre-Invasive Disease:Pathogenesis and Clinical Management,
Fitzgerald (Ed.), Springer, N.Y., 395-420,2011.
Mestdagh et al., Nucleic Acids Res., 36:e143, 2008.
Michael et al., Mo. Cancer Res., 1:882-891, 2003.
Monti et al., Virchows Arch., 445(3):236-247, 2004.
Nagula et al., J. Gastrointest. Surg., 14:1997-2003, 2010.
Olsen et al., Dev. Biol., 216:671, 1999.
Raval et al.; Mod. Pathol., 23:1467-1476, 2010.
Roggin et al., Curr. Probl. Surg., 47:459-510, 2010.
Ryu et al., Diagn. Cytopathol., 31:100-105, 2004.
Ryu et al., Pancreatology, 10:66-73, 2010.
Ryu et al., Pancreatology, 11:343-350, 2011.
Sambrook et al., In: DNA microarrays: a molecular cloning manual. Cold Spring Harbor
Laboratory Press, Cold Spring Harbor, N.Y., 2003.
Sambrook et al., In: Molecular cloning: a laboratory manual, 2^{nd} Ed., Cold Spring Harbor
Laboratory Press, Cold Spring Harbor, N.Y., 1989.
Sambrook et al., In: Molecular cloning: a laboratory manual, 3^{rd} Ed., Cold Spring Harbor
Laboratory Press, Cold Spring Harbor, N.Y., 2001.
Schmittgen et al., Methods, 44:31-38, 2008.
Schoedel et al., Diagn. Cytopathol., 34:605-608, 2006.
Seggerson et al., Dev. Biol., 243:215, 2002.
Szafranska et al. J. Mol. Diagn., 10:415-423, 2008.
Tanaka et al., Pancreatology, 6:17-32, 2006.
Tanaka, Nat. Rev. Gastroenterol. Hepatol., 8:56-60, 2011.
Tetzlaff et al., Endocr. Pathol., 18:163-173, 2007.
U.K. Patent 1,529,202
UK Appln. 8 803 000
Volinia et al., Proc. Natl. Acad. Sci. USA, 103:2257-2261, 2006.
Vriens et al., Cancer, 2011 DOI: 10.1002/cncr.26587
Winter et al., J. Gastrointest. Surg., 10:1199-1210, 2006.
WO 0.138580
WO 0168255
WO 03020898
WO 03022421
WO 03023058
WO 03029485
WO 03040410
WO 03053586
WO 03066906
WO 03067217
WO 03076928
WO 03087297
WO 03091426
WO 03093810
WO 03100012
WO 03100448A1
WO 04020085
WO 04027093
WO 09923256
WO 09936760
WO 93/17126
WO 95/11995
WO 95/21265
WO 95/21944
WO 95/35505
WO 96/31622
WO 97/10365
WO 97/27317
WO 9743450
WO 99135505
World Health Organization recommendations (WHO), Bosman et al., WHO Classification of Tumours of the Digestive System IARC. Lyon: 2010.
Wu et al., Sci. Trans. Med., 3:92ra66, 2011.
Xu et al., Cur: Biol., 13:790-795, 2003.
Yeo et al., Curr Prob Cancer, 26:176-275, 2002.

1-219. (canceled)

220. A method of treating a patient with a malignant thyroid tumor, the method comprising:
a) amplifying nucleic molecules from a patient thyroid sample;
b) measuring the level of expression in the thyroid sample of a group of microRNA selected from the group consisting of: miRNAs: miR-375, miR-146b-5p, miR-204-5p, miR-7-5p, and miR-141-3p;
c) comparing the level of expression of each biomarker miRNA measured in a) to the level of expression of another biomarker miRNA measured in a),
wherein mir-375 is compared to the level of expression of miR-7-5p, miR-204-5p, or miR-141-3p, or
wherein miR-146b-5p is compared to the level of expression of miR-7-5p, miR-204-5p, or miR-141-3p;
c) calculating a diagnostic score implemented by a computer that indicates the probability that the thyroid sample is malignant, wherein the diagnostic score is based on one or more of the comparisons in b),
wherein the sample is identified as malignant if the probability indicates the thyroid sample is malignant; and
treating the patient identified to have a malignant thyroid sample by administering radioactive iodine, radiation, chemotherapy to the patient identified to have a malignant thyroid sample or performing a full or partial thyroidectomy on the patient identified to have a malignant thyroid sample.

221. The method of claim 220, wherein the thyroid sample is a cystic fluid sample.

222. The method of claim 220, wherein the thyroid sample is a fine need aspirate sample.

223. The method of claim 220, wherein the diagnostic score is calculated using weighted coefficients for each of the measured expression levels of biomarker miRNAs.

224. The method of claim 220, wherein the diagnostic score identifies the thyroid sample as malignant when the expression level of at least one measured miRNA is increased in a malignant thyroid sample relative to the expression level of a benign thyroid sample and the expression level of at least a second measured miRNA is decreased in a malignant thyroid sample relative to the expression level of a benign thyroid sample,
wherein the increased miRNA is miR-141-3p, miR-146b-5p, or miR-375 and the decreased miRNA is miR-7-5p or miR-205-5p.

225. The method of claim 220, further comprising generating a malignant data set and a benign data set using logistic regression model and fitting the diagnostic score into either the malignant data set or the benign data set, wherein the diagnostic score that fits in the malignant data set is identified as a malignant thyroid sample or the diagnostic score that fits in the benign data set is identified as a benign thyroid sample.

226. The method of claim 220, wherein the treating comprises administering radioactive iodine to the patient identified to have a malignant thyroid sample.

227. The method of claim 220, wherein the treating comprises administering radiation to the patient identified to have a malignant thyroid sample.

228. The method of claim 220, wherein the treating comprises administering chemotherapy to the patient identified to have a malignant thyroid sample.

229. The method of claim 220, wherein the treating comprises performing a partial thyroidectomy on the patient identified to have a malignant thyroid sample.

230. The method of claim 220, wherein the treating comprises performing a full thyroidectomy on the patient identified to have a malignant thyroid sample.

231. A method of producing a labeled set of nucleic acid molecules, the method comprising:
a) amplifying nucleic molecules from a patient thyroid sample; and
b) labeling a group of microRNA molecules selected from the group consisting of: miRNAs: miR-375, miR-146b-5p, miR-204-5p, miR-7-5p, and miR-141-3p.

232. The method of claim 231, wherein the label is a fluorescent label.

234. The method of claim 231, wherein the amplifying step labels the group of microRNA.

235. The method of claim 231, wherein the amplifying step comprises contacting the sample with labeled primers.

236. The method of claim 235, wherein the labeled primers are fluorescent labeled primers.


SEQUENCE LISTING (1164 sequences)

SEQ ID NO: 1 - RNA, 22 nt, Homo sapiens
ggagaaauuauccuuggugugu

SEQ ID NO: 2 - RNA, 23 nt, Homo sapiens
uggaagacuagugauuuuguugu

SEQ ID NO: 3 - RNA, 22 nt, Homo sapiens
ugagaacugaauuccauaggcu

SEQ ID NO: 4 - RNA, 22 nt, Homo sapiens
ugcccuguggacucaguucugg

SEQ ID NO: 5 - RNA, 22 nt, Homo sapiens
aacacaccugguuaaccucuuu

SEQ ID NO: 6 - RNA, 22 nt, Homo sapiens
ucggauccgucugagcuuggcu

SEQ ID NO: 7 - RNA, 22 nt, Homo sapiens
accuggcauacaauguagauuu

SEQ ID NO: 8 - RNA, 21 nt, Homo sapiens
ccaguccugugccugccgccu

SEQ ID NO: 9 - RNA, 22 nt, Homo sapiens
aaacaaacauggugcacuucuu

SEQ ID NO: 10 - RNA, 22 nt, Homo sapiens
gaauguugcucggugaaccccu

SEQ ID NO: 11 - RNA, 21 nt, Homo sapiens
aacauagaggaaauuccacgu

SEQ ID NO: 12 - RNA, 22 nt, Homo sapiens
uccuucauuccaccggagucug

SEQ ID NO: 13 - RNA, 22 nt, Homo sapiens
uuuguucguucggcucgcguga

SEQ ID NO: 14 - RNA, 22 nt, Homo sapiens
cucaguagccaguguagauccu

SEQ ID NO: 15 - RNA, 22 nt, Homo sapiens
agaucgaccguguuauauucgc

SEQ ID NO: 16 - RNA, 23 nt, Homo sapiens
ugucugcccgcaugccugccucu

SEQ ID NO: 17 - RNA, 23 nt, Homo sapiens
agcuacauugucugcuggguuuc

SEQ ID NO: 18 - RNA, 21 nt, Homo sapiens
aggcaagaugcuggcauagcu

SEQ ID NO: 19 - RNA, 22 nt, Homo sapiens
uuguacaugguaggcuuucauu

SEQ ID NO: 20 - RNA, 22 nt, Homo sapiens
aaucugagaaggcgcacaaggu

SEQ ID NO: 21 - RNA, 22 nt, Homo sapiens
caccuugcgcuacucaggucug

SEQ ID NO: 22 - RNA, 22 nt, Homo sapiens
uagcuuaucagacugauguuga

SEQ ID NO: 23 - RNA, 22 nt, Homo sapiens
uggcagugucuuagcugguugu

SEQ ID NO: 24 - RNA, 22 nt, Homo sapiens
gcuacuucacaacaccagggcc

SEQ ID NO: 25 - RNA, 22 nt, Homo sapiens
ucuacagugcacgugucuccag

SEQ ID NO: 26 - RNA, 22 nt, Homo sapiens
uaacacugucugguaaagaugg

SEQ ID NO: 27 - RNA, 23 nt, Homo sapiens
guccaguuuucccaggaaucccu

SEQ ID NO: 28 - RNA, 23 nt, Homo sapiens
uuaaugcuaaucgugauaggggu

SEQ ID NO: 29 - RNA, 22 nt, Homo sapiens
uauggcacugguagaauucacu

SEQ ID NO: 30 - RNA, 22 nt, Homo sapiens
uucaccaccuucuccacccagc

SEQ ID NO: 31 - RNA, 21 nt, Homo sapiens
agcuacaucuggcuacugggu

SEQ ID NO: 32 - RNA, 21 nt, Homo sapiens
caagucacuagugguuccguu

SEQ ID NO: 33 - RNA, 23 nt, Homo sapiens
aaugacacgaucacucccguuga

SEQ ID NO: 34 - RNA, 22 nt, Homo sapiens
ugagaacugaauuccauggguu

SEQ ID NO: 35 - RNA, 23 nt, Homo sapiens
uacccuguagaaccgaauuugug

SEQ ID NO: 36 - RNA, 22 nt, Homo sapiens
uagcagcacauaaugguuugug

SEQ ID NO: 37 - RNA, 22 nt, Homo sapiens
ugccuacugagcugauaucagu

SEQ ID NO: 38 - RNA, 22 nt, Homo sapiens
ugccuacugagcugaaacacag

SEQ ID NO: 39 - RNA, 21 nt, Homo sapiens
uucacaguggcuaaguuccgc

SEQ ID NO: 40 - RNA, 22 nt, Homo sapiens
agagcuuagcugauuggugaac

SEQ ID NO: 41 - RNA, 22 nt, Homo sapiens
ugcuaugccaacauauugccau

SEQ ID NO: 42 - RNA, 22 nt, Homo sapiens
caaucagcaaguauacugcccu

SEQ ID NO: 43 - RNA, 23 nt, Homo sapiens
uaggcagugucauuagcugauug

SEQ ID NO: 44 - RNA, 22 nt, Homo sapiens
uaacagucuacagccauggucg

SEQ ID NO: 45 - RNA, 22 nt, Homo sapiens
accguggcuuucgauuguuacu

SEQ ID NO: 46 - RNA, 21 nt, Homo sapiens
cauaaaguagaaagcacuacu

SEQ ID NO: 47 - RNA, 22 nt, Homo sapiens
accaucgaccguugauuguacc

SEQ ID NO: 48 - RNA, 22 nt, Homo sapiens
aacauucaaccugucggugagu

SEQ ID NO: 49 - RNA, 24 nt, Homo sapiens
uuuggcaaugguagaacucacacu

SEQ ID NO: 50 - RNA, 22 nt, Homo sapiens
uagguaguuucauguuguuggg

SEQ ID NO: 51 - RNA, 23 nt, Homo sapiens
cagugcaauaguauugucaaagc

SEQ ID NO: 52 - RNA, 23 nt, Homo sapiens
ucaagagcaauaacgaaaaaugu

SEQ ID NO: 53 - RNA, 22 nt, Homo sapiens
uuauaaagcaaugagacugauu

SEQ ID NO: 54 - RNA, 21 nt, Homo sapiens
gcgacccauacuugguuucag

SEQ ID NO: 55 - RNA, 18 nt, Homo sapiens
ugaggcaguagauugaau

SEQ ID NO: 56 - RNA, 23 nt, Homo sapiens
ucaacaaaaucacugaugcugga

SEQ ID NO: 57 - RNA, 17 nt, Homo sapiens
uugucugcugaguuucc

SEQ ID NO: 58 - RNA, 22 nt, Homo sapiens
aggggcuggcuuuccucugguc

SEQ ID NO: 59 - RNA, 17 nt, Homo sapiens
cagagaauuguuuaauc

SEQ ID NO: 60 - RNA, 21 nt, Homo sapiens
ccaguccugugccugccgccu

SEQ ID NO: 61 - RNA, 22 nt, Homo sapiens
uccuguacugagcugccccgag

SEQ ID NO: 62 - RNA, 22 nt, Homo sapiens
aaagugcauccuuuuagagugu

SEQ ID NO: 63 - RNA, 21 nt, Homo sapiens
cauaaaguagaaagcacuacu

SEQ ID NO: 64 - RNA, 18 nt, Homo sapiens
gucccuguucaggcgcca

SEQ ID NO: 65 - RNA, 22 nt, Homo sapiens
ugagguaguagguuguauaguu

SEQ ID NO: 66 - RNA, 21 nt, Homo sapiens
cuauacaaucuacugucuuuc

SEQ ID NO: 67 - RNA, 22 nt, Homo sapiens
cuguacagccuccuagcuuucc

SEQ ID NO: 68 - RNA, 22 nt, Homo sapiens
ugagguaguagguugugugguu

SEQ ID NO: 69 - RNA, 22 nt, Homo sapiens
cuauacaaccuacugccuuccc

SEQ ID NO: 70 - RNA, 22 nt, Homo sapiens
ugagguaguagguuguaugguu

SEQ ID NO: 71 - RNA, 22 nt, Homo sapiens
uagaguuacacccugggaguua

SEQ ID NO: 72 - RNA, 22 nt, Homo sapiens
agagguaguagguugcauaguu

SEQ ID NO: 73 - RNA, 22 nt, Homo sapiens
cuauacgaccugcugccuuucu

SEQ ID NO: 74 - RNA, 22 nt, Homo sapiens
ugagguaggagguuguauaguu

SEQ ID NO: 75 - RNA, 22 nt, Homo sapiens
cuauacggccuccuagcuuucc

SEQ ID NO: 76 - RNA, 22 nt, Homo sapiens
ugagguaguagauuguauaguu

SEQ ID NO: 77 - RNA, 22 nt, Homo sapiens
cuauacaaucuauugccuuccc

SEQ ID NO: 78 - RNA, 22 nt, Homo sapiens
cuauacagucuacugucuuucc

SEQ ID NO: 79 - RNA, 22 nt, Homo sapiens
uagcagcacauaaugguuugug

SEQ ID NO: 80 - RNA, 22 nt, Homo sapiens
caggccauauugugcugccuca

SEQ ID NO: 81 - RNA, 22 nt, Homo sapiens
uagcagcacguaaauauuggcg

SEQ ID NO: 82 - RNA, 22 nt, Homo sapiens
ccaguauuaacugugcugcuga

SEQ ID NO: 83 - RNA, 23 nt, Homo sapiens
caaagugcuuacagugcagguag

SEQ ID NO: 84 - RNA, 22 nt, Homo sapiens
acugcagugaaggcacuuguag

SEQ ID NO: 85 - RNA, 23 nt, Homo sapiens
uaaggugcaucuagugcagauag

SEQ ID NO: 86 - RNA, 23 nt, Homo sapiens
acugcccuaagugcuccuucugg

SEQ ID NO: 87 - RNA, 22 nt, Homo sapiens
aguuuugcauaguugcacuaca

SEQ ID NO: 88 - RNA, 23 nt, Homo sapiens
ugugcaaaucuaugcaaaacuga

SEQ ID NO: 89 - RNA, 23 nt, Homo sapiens
aguuuugcagguuugcauccagc

SEQ ID NO: 90 - RNA, 23 nt, Homo sapiens
ugugcaaauccaugcaaaacuga

SEQ ID NO: 91 - RNA, 22 nt, Homo sapiens
aguuuugcagguuugcauuuca

SEQ ID NO: 92 - RNA, 23 nt, Homo sapiens
uaaagugcuuauagugcagguag

SEQ ID NO: 93 - RNA, 22 nt, Homo sapiens
acugcauuaugagcacuuaaag

SEQ ID NO: 94 - RNA, 22 nt, Homo sapiens
uagcuuaucagacugauguuga

SEQ ID NO: 95 - RNA, 21 nt, Homo sapiens
caacaccagucgaugggcugu

SEQ ID NO: 96 - RNA, 22 nt, Homo sapiens
aguucuucaguggcaagcuuua

SEQ ID NO: 97 - RNA, 22 nt, Homo sapiens
aagcugccaguugaagaacugu

SEQ ID NO: 98 - RNA, 22 nt, Homo sapiens
gggguuccuggggaugggauuu

SEQ ID NO: 99 - RNA, 21 nt, Homo sapiens
aucacauugccagggauuucc

SEQ ID NO: 100 - RNA, 22 nt, Homo sapiens
ugccuacugagcugauaucagu

SEQ ID NO: 101 - RNA, 22 nt, Homo sapiens
uggcucaguucagcaggaacag

SEQ ID NO: 102 - RNA, 22 nt, Homo sapiens
ugccuacugagcugaaacacag

SEQ ID NO: 103 - RNA, 21 nt, Homo sapiens
aggcggagacuugggcaauug

SEQ ID NO: 104 - RNA, 22 nt, Homo sapiens
cauugcacuugucucggucuga

SEQ ID NO: 105 - RNA, 22 nt, Homo sapiens
uucaaguaauccaggauaggcu

SEQ ID NO: 106 - RNA, 22 nt, Homo sapiens
ccuauucuugguuacuugcacg

SEQ ID NO: 107 - RNA, 21 nt, Homo sapiens
uucaaguaauucaggauaggu

SEQ ID NO: 108 - RNA, 22 nt, Homo sapiens
ccuguucuccauuacuuggcuc

SEQ ID NO: 109 - RNA, 22 nt, Homo sapiens
agggcuuagcugcuugugagca

SEQ ID NO: 110 - RNA, 21 nt, Homo sapiens
uucacaguggcuaaguuccgc

SEQ ID NO: 111 - RNA, 22 nt, Homo sapiens
aaggagcucacagucuauugag

SEQ ID NO: 112 - RNA, 22 nt, Homo sapiens
cacuagauugugagcuccugga

SEQ ID NO: 113 - RNA, 22 nt, Homo sapiens
acugauuucuuuugguguucag

SEQ ID NO: 114 - RNA, 22 nt, Homo sapiens
uagcaccaucugaaaucgguua

SEQ ID NO: 115 - RNA, 22 nt, Homo sapiens
uguaaacauccucgacuggaag

SEQ ID NO: 116 - RNA, 22 nt, Homo sapiens
cuuucagucggauguuugcagc

SEQ ID NO: 117 - RNA, 21 nt, Homo sapiens
aggcaagaugcuggcauagcu

SEQ ID NO: 118 - RNA, 22 nt, Homo sapiens
ugcuaugccaacauauugccau

SEQ ID NO: 119 - RNA, 22 nt, Homo sapiens
uauugcacauuacuaaguugca

SEQ ID NO: 120 - RNA, 22 nt, Homo sapiens
caauuuagugugugugauauuu

SEQ ID NO: 121 - RNA, 21 nt, Homo sapiens
gugcauuguaguugcauugca

SEQ ID NO: 122 - RNA, 22 nt, Homo sapiens
caauguuuccacagugcaucac

SEQ ID NO: 123 - RNA, 23 nt, Homo sapiens
agguugggaucgguugcaaugcu

SEQ ID NO: 124 - RNA, 22 nt, Homo sapiens
uauugcacuugucccggccugu

SEQ ID NO: 125 - RNA, 22 nt, Homo sapiens
ggguggggauuuguugcauuac

SEQ ID NO: 126 - RNA, 23 nt, Homo sapiens
caaagugcuguucgugcagguag

SEQ ID NO: 127 - RNA, 22 nt, Homo sapiens
acugcugagcuagcacuucccg

SEQ ID NO: 128 - RNA, 22 nt, Homo sapiens
uucaacggguauuuauugagca

SEQ ID NO: 129 - RNA, 23 nt, Homo sapiens
uuuggcacuagcacauuuuugcu

SEQ ID NO: 130 - RNA, 22 nt, Homo sapiens
aaucaugugcagugccaauaug

SEQ ID NO: 131 - RNA, 22 nt, Homo sapiens
ugagguaguaaguuguauuguu

SEQ ID NO: 132 - RNA, 22 nt, Homo sapiens
aacccguagauccgaucuugug

SEQ ID NO: 133 - RNA, 22 nt, Homo sapiens
caagcucgcuucuaugggucug

SEQ ID NO: 134 - RNA, 22 nt, Homo sapiens
aacccguagauccgaacuugug

SEQ ID NO: 135 - RNA, 22 nt, Homo sapiens
caagcuuguaucuauagguaug

SEQ ID NO: 136 - RNA, 22 nt, Homo sapiens
caguuaucacagugcugaugcu

SEQ ID NO: 137 - RNA, 21 nt, Homo sapiens
uacaguacugugauaacugaa

SEQ ID NO: 138 - RNA, 24 nt, Homo sapiens
gcugguuucauauggugguuuaga

SEQ ID NO: 139 - RNA, 23 nt, Homo sapiens
uagcaccauuugaaaucaguguu

SEQ ID NO: 140 - RNA, 22 nt, Homo sapiens
cugguuucacaugguggcuuag

SEQ ID NO: 141 - RNA, 23 nt, Homo sapiens
agcuucuuuacagugcugccuug

SEQ ID NO: 142 - RNA, 23 nt, Homo sapiens
agcagcauuguacagggcuauga

SEQ ID NO: 143 - RNA, 23 nt, Homo sapiens
ucaaaugcucagacuccuguggu

SEQ ID NO: 144 - RNA, 22 nt, Homo sapiens
acggauguuugagcaugugcua

SEQ ID NO: 145 - RNA, 23 nt, Homo sapiens
aaaagugcuuacagugcagguag

SEQ ID NO: 146 - RNA, 22 nt, Homo sapiens
cugcaauguaagcacuucuuac

SEQ ID NO: 147 - RNA, 23 nt, Homo sapiens
agcagcauuguacagggcuauca

SEQ ID NO: 148 - RNA, 22 nt, Homo sapiens
ccaauauuacugugcugcuuua

SEQ ID NO: 149 - RNA, 21 nt, Homo sapiens
cugaccuaugaauugacagcc

SEQ ID NO: 150 - RNA, 22 nt, Homo sapiens
cugccaauuccauaggucacag

SEQ ID NO: 151 - RNA, 22 nt, Homo sapiens
uagguaguuucauguuguuggg

SEQ ID NO: 152 - RNA, 22 nt, Homo sapiens
uucaccaccuucuccacccagc

SEQ ID NO: 153 - RNA, 22 nt, Homo sapiens
gguccagaggggagauagguuc

SEQ ID NO: 154 - RNA, 23 nt, Homo sapiens
cccaguguucagacuaccuguuc

SEQ ID NO: 155 - RNA, 22 nt, Homo sapiens
acaguagucugcacauugguua

SEQ ID NO: 156 - RNA, 22 nt, Homo sapiens
auaagacgagcaaaaagcuugu

SEQ ID NO: 157 - RNA, 21 nt, Homo sapiens
cuuuuugcggucugggcuugc

SEQ ID NO: 158 - RNA, 22 nt, Homo sapiens
aagcccuuaccccaaaaaguau

SEQ ID NO: 159 - RNA, 22 nt, Homo sapiens
aaaguucugagacacuccgacu

SEQ ID NO: 160 - RNA, 22 nt, Homo sapiens
ucagugcacuacagaacuuugu

SEQ ID NO: 161 - RNA, 23 nt, Homo sapiens
uguaaacauccuacacucucagc

SEQ ID NO: 162 - RNA, 22 nt, Homo sapiens
cugggagaaggcuguuuacucu

SEQ ID NO: 163 - RNA, 22 nt, Homo sapiens
uguaaacauccccgacuggaag

SEQ ID NO: 164 - RNA, 22 nt, Homo sapiens
cuuucagucagauguuugcugc

SEQ ID NO: 165 - RNA, 22 nt, Homo sapiens
ucuacagugcacgugucuccag

SEQ ID NO: 166 - RNA, 22 nt, Homo sapiens
ggagacgcggcccuguuggagu

SEQ ID NO: 167 - RNA, 20 nt, Homo sapiens
guguguggaaaugcuucugc

SEQ ID NO: 168 - RNA, 23 nt, Homo sapiens
uggaagacuagugauuuuguugu

SEQ ID NO: 169 - RNA, 22 nt, Homo sapiens
caacaaaucacagucugccaua

SEQ ID NO: 170 - RNA, 22 nt, Homo sapiens
caacaaaucccagucuaccuaa

SEQ ID NO: 171 - RNA, 23 nt, Homo sapiens
uacccuguagauccgaauuugug

SEQ ID NO: 172 - RNA, 22 nt, Homo sapiens
caaauucguaucuaggggaaua

SEQ ID NO: 173 - RNA, 23 nt, Homo sapiens
uacccuguagaaccgaauuugug

SEQ ID NO: 174 - RNA, 22 nt, Homo sapiens
acagauucgauucuaggggaau

SEQ ID NO: 175 - RNA, 22 nt, Homo sapiens
uggcagugucuuagcugguugu

SEQ ID NO: 176 - RNA, 22 nt, Homo sapiens
caaucagcaaguauacugcccu

SEQ ID NO: 177 - RNA, 23 nt, Homo sapiens
aacauucaacgcugucggugagu

SEQ ID NO: 178 - RNA, 22 nt, Homo sapiens
accacugaccguugacuguacc

SEQ ID NO: 179 - RNA, 23 nt, Homo sapiens
aacauucauugcugucggugggu

SEQ ID NO: 180 - RNA, 22 nt, Homo sapiens
aacauucaaccugucggugagu

SEQ ID NO: 181 - RNA, 22 nt, Homo sapiens
aaccaucgaccguugaguggac

SEQ ID NO: 182 - RNA, 24 nt, Homo sapiens
uuuggcaaugguagaacucacacu

SEQ ID NO: 183 - RNA, 21 nt, Homo sapiens
ugguucuagacuugccaacua

SEQ ID NO: 184 - RNA, 22 nt, Homo sapiens
uauggcacugguagaauucacu

SEQ ID NO: 185 - RNA, 22 nt, Homo sapiens
gugaauuaccgaagggccauaa

SEQ ID NO: 186 - RNA, 22 nt, Homo sapiens
ggcuacaacacaggacccgggc

SEQ ID NO: 187 - RNA, 22 nt, Homo sapiens
ucgugucuuguguugcagccgg

SEQ ID NO: 188 - RNA, 22 nt, Homo sapiens
cggcaacaagaaacugccugag

SEQ ID NO: 189 - RNA, 23 nt, Homo sapiens
cccaguguuuagacuaucuguuc

SEQ ID NO: 190 - RNA, 22 nt, Homo sapiens
acaguagucugcacauugguua

SEQ ID NO: 191 - RNA, 22 nt, Homo sapiens
gugaaauguuuaggaccacuag

SEQ ID NO: 192 - RNA, 22 nt, Homo sapiens
uucccuuugucauccuaugccu

SEQ ID NO: 193 - RNA, 22 nt, Homo sapiens
uccuucauuccaccggagucug

SEQ ID NO: 194 - RNA, 21 nt, Homo sapiens
gauuucaguggagugaaguuc

SEQ ID NO: 195 - RNA, 22 nt, Homo sapiens
cugugcgugugacagcggcuga

SEQ ID NO: 196 - RNA, 22 nt, Homo sapiens
uucccuuugucauccuucgccu

SEQ ID NO: 197 - RNA, 21 nt, Homo sapiens
uaacagucuccagucacggcc

SEQ ID NO: 198 - RNA, 22 nt, Homo sapiens
accaucgaccguugauuguacc

SEQ ID NO: 199 - RNA, 22 nt, Homo sapiens
ugccugucuacacuugcugugc

SEQ ID NO: 200 - RNA, 22 nt, Homo sapiens
acagcaggcacagacaggcagu

SEQ ID NO: 201 - RNA, 21 nt, Homo sapiens
augaccuaugaauugacagac

SEQ ID NO: 202 - RNA, 22 nt, Homo sapiens
uaaucucagcuggcaacuguga

SEQ ID NO: 203 - RNA, 23 nt, Homo sapiens
uacugcaucaggaacugauugga

SEQ ID NO: 204 - RNA, 21 nt, Homo sapiens
uugugcuugaucuaaccaugu

SEQ ID NO: 205 - RNA, 22 nt, Homo sapiens
augguuccgucaagcaccaugg

SEQ ID NO: 206 - RNA, 22 nt, Homo sapiens
caugguucugucaagcaccgcg

SEQ ID NO: 207 - RNA, 21 nt, Homo sapiens
ugauuguccaaacgcaauucu

SEQ ID NO: 208 - RNA, 22 nt, Homo sapiens
agaguugagucuggacgucccg

SEQ ID NO: 209 - RNA, 21 nt, Homo sapiens
ccacaccguaucugacacuuu

SEQ ID NO: 210 - RNA, 22 nt, Homo sapiens
accuggcauacaauguagauuu

SEQ ID NO: 211 - RNA, 23 nt, Homo sapiens
agcuacauugucugcuggguuuc

SEQ ID NO: 212 - RNA, 22 nt, Homo sapiens
cucaguagccaguguagauccu

SEQ ID NO: 213 - RNA, 21 nt, Homo sapiens
agcuacaucuggcuacugggu

SEQ ID NO: 214 - RNA, 22 nt, Homo sapiens
cguguauuugacaagcugaguu

SEQ ID NO: 215 - RNA, 22 nt, Homo sapiens
ugucaguuugucaaauacccca

SEQ ID NO: 216 - RNA, 21 nt, Homo sapiens
caagucacuagugguuccguu

SEQ ID NO: 217 - RNA, 23 nt, Homo sapiens
aaaauggugcccuagugacuaca

SEQ ID NO: 218 - RNA, 22 nt, Homo sapiens
caucuuacugggcagcauugga

SEQ ID NO: 219 - RNA, 22 nt, Homo sapiens
uaauacugccugguaaugauga

SEQ ID NO: 220 - RNA, 22 nt, Homo sapiens
ugagguaguaguuuguacaguu

SEQ ID NO: 221 - RNA, 21 nt, Homo sapiens
cuguacaggccacugccuugc

SEQ ID NO: 222 - RNA, 22 nt, Homo sapiens
ugagguaguaguuugugcuguu

SEQ ID NO: 223 - RNA, 22 nt, Homo sapiens
cugcgcaagcuacugccuugcu

SEQ ID NO: 224 - RNA, 22 nt, Homo sapiens
uggaauguaaagaaguauguau

SEQ ID NO: 225 - RNA, 22 nt, Homo sapiens
uagcagcacaucaugguuuaca

SEQ ID NO: 226 - RNA, 22 nt, Homo sapiens
cgaaucauuauuugcugcucua

SEQ ID NO: 227 - RNA, 22 nt, Homo sapiens
uggguuccuggcaugcugauuu

SEQ ID NO: 228 - RNA, 21 nt, Homo sapiens
aucacauugccagggauuacc

SEQ ID NO: 229 - RNA, 22 nt, Homo sapiens
agagcuuagcugauuggugaac

SEQ ID NO: 230 - RNA, 21 nt, Homo sapiens
uucacaguggcuaaguucugc

SEQ ID NO: 231 - RNA, 22 nt, Homo sapiens
uguaaacauccuacacucagcu

SEQ ID NO: 232 - RNA, 22 nt, Homo sapiens
cugggagguggauguuuacuuc

SEQ ID NO: 233 - RNA, 22 nt, Homo sapiens
uggagugugacaaugguguuug

SEQ ID NO: 234 - RNA, 22 nt, Homo sapiens
aacgccauuaucacacuaaaua

SEQ ID NO: 235 - RNA, 22 nt, Homo sapiens
cguguucacagcggaccuugau

SEQ ID NO: 236 - RNA, 20 nt, Homo sapiens
uaaggcacgcggugaaugcc

SEQ ID NO: 237 - RNA, 22 nt, Homo sapiens
ucccugagacccuaacuuguga

SEQ ID NO: 238 - RNA, 22 nt, Homo sapiens
acggguuaggcucuugggagcu

SEQ ID NO: 239 - RNA, 21 nt, Homo sapiens
ucacagugaaccggucucuuu

SEQ ID NO: 240 - RNA, 22 nt, Homo sapiens
uucacauugugcuacugucugc

SEQ ID NO: 241 - RNA, 22 nt, Homo sapiens
cagugcaauguuaaaagggcau

SEQ ID NO: 242 - RNA, 22 nt, Homo sapiens
accguggcuuucgauuguuacu

SEQ ID NO: 243 - RNA, 22 nt, Homo sapiens
uaacagucuacagccauggucg

SEQ ID NO: 244 - RNA, 22 nt, Homo sapiens
uuugguccccuucaaccagcug

SEQ ID NO: 245 - RNA, 23 nt, Homo sapiens
uauggcuuuuuauuccuauguga

SEQ ID NO: 246 - RNA, 22 nt, Homo sapiens
uauagggauuggagccguggcg

SEQ ID NO: 247 - RNA, 23 nt, Homo sapiens
uuauugcuuaagaauacgcguag

SEQ ID NO: 248 - RNA, 23 nt, Homo sapiens
agcugguguugugaaucaggccg

SEQ ID NO: 249 - RNA, 22 nt, Homo sapiens
gcuauuucacgacaccaggguu

SEQ ID NO: 250 - RNA, 22 nt, Homo sapiens
cagugguuuuacccuaugguag

SEQ ID NO: 251 - RNA, 21 nt, Homo sapiens
uaccacaggguagaaccacgg

SEQ ID NO: 252 - RNA, 22 nt, Homo sapiens
caucuuccaguacaguguugga

SEQ ID NO: 253 - RNA, 22 nt, Homo sapiens
uaacacugucugguaaagaugg

SEQ ID NO: 254 - RNA, 21 nt, Homo sapiens
cauaaaguagaaagcacuacu

SEQ ID NO: 255 - RNA, 23 nt, Homo sapiens
uguaguguuuccuacuuuaugga

SEQ ID NO: 256 - RNA, 22 nt, Homo sapiens
ggugcagugcugcaucucuggu

SEQ ID NO: 257 - RNA, 21 nt, Homo sapiens
ugagaugaagcacuguagcuc

SEQ ID NO: 258 - RNA, 22 nt, Homo sapiens
ggauaucaucauauacuguaag

SEQ ID NO: 259 - RNA, 20 nt, Homo sapiens
uacaguauagaugauguacu

SEQ ID NO: 260 - RNA, 23 nt, Homo sapiens
guccaguuuucccaggaaucccu

SEQ ID NO: 261 - RNA, 22 nt, Homo sapiens
ggauuccuggaaauacuguucu

SEQ ID NO: 262 - RNA, 21 nt, Homo sapiens
ucagugcaugacagaacuugg

SEQ ID NO: 263 - RNA, 22 nt, Homo sapiens
uugcauagucacaaaagugauc

SEQ ID NO: 264 - RNA, 23 nt, Homo sapiens
caacggaaucccaaaagcagcug

SEQ ID NO: 265 - RNA, 22 nt, Homo sapiens
gcugcgcuuggauuucgucccc

SEQ ID NO: 266 - RNA, 23 nt, Homo sapiens
ucuuugguuaucuagcuguauga

SEQ ID NO: 267 - RNA, 22 nt, Homo sapiens
auaaagcuagauaaccgaaagu

SEQ ID NO: 268 - RNA, 24 nt, Homo sapiens
ucccugagacccuuuaaccuguga

SEQ ID NO: 269 - RNA, 22 nt, Homo sapiens
acaggugagguucuugggagcc

SEQ ID NO: 270 - RNA, 22 nt, Homo sapiens
ucacaagucaggcucuugggac

SEQ ID NO: 271 - RNA, 21 nt, Homo sapiens
cauuauuacuuuugguacgcg

SEQ ID NO: 272 - RNA, 22 nt, Homo sapiens
ucguaccgugaguaauaaugcg

SEQ ID NO: 273 - RNA, 22 nt, Homo sapiens
cugaagcucagagggcucugau

SEQ ID NO: 274 - RNA, 22 nt, Homo sapiens
ucggauccgucugagcuuggcu

SEQ ID NO: 275 - RNA, 22 nt, Homo sapiens
aagcccuuaccccaaaaagcau

SEQ ID NO: 276 - RNA, 22 nt, Homo sapiens
ugugacugguugaccagagggg

SEQ ID NO: 277 - RNA, 23 nt, Homo sapiens
acuccauuuguuuugaugaugga

SEQ ID NO: 278 - RNA, 22 nt, Homo sapiens
caucaucgucucaaaugagucu

SEQ ID NO: 279 - RNA, 22 nt, Homo sapiens
gcuacuucacaacaccagggcc

SEQ ID NO: 280 - RNA, 22 nt, Homo sapiens
ugagaacugaauuccauggguu

SEQ ID NO: 281 - RNA, 22 nt, Homo sapiens
ccucugaaauucaguucuucag

SEQ ID NO: 282 - RNA, 23 nt, Homo sapiens
ucuggcuccgugucuucacuccc

SEQ ID NO: 283 - RNA, 21 nt, Homo sapiens
agggagggacgggggcugugc

SEQ ID NO: 284 - RNA, 22 nt, Homo sapiens
ucucccaacccuuguaccagug

SEQ ID NO: 285 - RNA, 22 nt, Homo sapiens
cugguacaggccugggggacag

SEQ ID NO: 286 - RNA, 22 nt, Homo sapiens
uagguuauccguguugccuucg

SEQ ID NO: 287 - RNA, 22 nt, Homo sapiens
aaucauacacgguugaccuauu

SEQ ID NO: 288 - RNA, 22 nt, Homo sapiens
uggacggagaacugauaagggu

SEQ ID NO: 289 - RNA, 22 nt, Homo sapiens
uggagagaaaggcaguuccuga

SEQ ID NO: 290 - RNA, 22 nt, Homo sapiens
aggggcuggcuuuccucugguc

SEQ ID NO: 291 - RNA, 22 nt, Homo sapiens
caaagaauucuccuuuugggcu

SEQ ID NO: 292 - RNA, 22 nt, Homo sapiens
gcccaaaggugaauuuuuuggg

SEQ ID NO: 293 - RNA, 21 nt, Homo sapiens
caucccuugcaugguggaggg

SEQ ID NO: 294 - RNA, 21 nt, Homo sapiens
cucccacaugcaggguuugca

SEQ ID NO: 295 - RNA, 22 nt, Homo sapiens
ugauauguuugauauauuaggu

SEQ ID NO: 296 - RNA, 22 nt, Homo sapiens
ugggucuuugcgggcgagauga

SEQ ID NO: 297 - RNA, 22 nt, Homo sapiens
aacuggccuacaaagucccagu

SEQ ID NO: 298 - RNA, 22 nt, Homo sapiens
uguaacagcaacuccaugugga

SEQ ID NO: 299 - RNA, 21 nt, Homo sapiens
uagcagcacagaaauauuggc

SEQ ID NO: 300 - RNA, 22 nt, Homo sapiens
ccaauauuggcugugcugcucc

SEQ ID NO: 301 - RNA, 22 nt, Homo sapiens
uggaauguaaggaagugugugg

SEQ ID NO: 302 - RNA, 22 nt, Homo sapiens
aaaagcuggguugagagggcga

SEQ ID NO: 303 - RNA, 22 nt, Homo sapiens
cgucuuacccagcaguguuugg

SEQ ID NO: 304 - RNA, 23 nt, Homo sapiens
uaauacugccggguaaugaugga

SEQ ID NO: 305 - RNA, 23 nt, Homo sapiens
uuaaugcuaaucgugauaggggu

SEQ ID NO: 306 - RNA, 22 nt, Homo sapiens
cuccuacauauuagcauuaaca

SEQ ID NO: 307 - RNA, 22 nt, Homo sapiens
ccaguggggcugcuguuaucug

SEQ ID NO: 308 - RNA, 21 nt, Homo sapiens
uaaagugcugacagugcagau

SEQ ID NO: 309 - RNA, 22 nt, Homo sapiens
ccgcacuguggguacuugcugc

SEQ ID NO: 310 - RNA, 22 nt, Homo sapiens
ugaccgauuucuccugguguuc

SEQ ID NO: 311 - RNA, 22 nt, Homo sapiens
uagcaccauuugaaaucgguua

SEQ ID NO: 312 - RNA, 22 nt, Homo sapiens
cugggagaggguuguuuacucc

SEQ ID NO: 313 - RNA, 22 nt, Homo sapiens
caucuuaccggacagugcugga

SEQ ID NO: 314 - RNA, 22 nt, Homo sapiens
uaacacugucugguaacgaugu

SEQ ID NO: 315 - RNA, 23 nt, Homo sapiens
acuuaaacguggauguacuugcu

SEQ ID NO: 316 - RNA, 23 nt, Homo sapiens
uaagugcuuccauguuuugguga

SEQ ID NO: 317 - RNA, 22 nt, Homo sapiens
agaauuguggcuggacaucugu

SEQ ID NO: 318 - RNA, 23 nt, Homo sapiens
uaggcagugucauuagcugauug

SEQ ID NO: 319 - RNA, 22 nt, Homo sapiens
caaucacuaacuccacugccau

SEQ ID NO: 320 - RNA, 23 nt, Homo sapiens
aggcaguguaguuagcugauugc

SEQ ID NO: 321 - RNA, 22 nt, Homo sapiens
aaucacuaaccacacggccagg

SEQ ID NO: 322 - RNA, 22 nt, Homo sapiens
ugguuuaccgucccacauacau

SEQ ID NO: 323 - RNA, 22 nt, Homo sapiens
uaugugggaugguaaaccgcuu

SEQ ID NO: 324 - RNA, 23 nt, Homo sapiens
cagugcaauaguauugucaaagc

SEQ ID NO: 325 - RNA, 22 nt, Homo sapiens
cacccguagaaccgaccuugcg

SEQ ID NO: 326 - RNA, 22 nt, Homo sapiens
caagcucgugucuguggguccg

SEQ ID NO: 327 - RNA, 21 nt, Homo sapiens
agggcccccccucaauccugu

SEQ ID NO: 328 - RNA, 22 nt, Homo sapiens
gaggguuggguggaggcucucc

SEQ ID NO: 329 - RNA, 21 nt, Homo sapiens
acucuuucccuguugcacuac

SEQ ID NO: 330 - RNA, 22 nt, Homo sapiens
cagugcaaugaugaaagggcau

SEQ ID NO: 331 - RNA, 22 nt, Homo sapiens
uguaaacauccuugacuggaag

SEQ ID NO: 332 - RNA, 22 nt, Homo sapiens
cuuucagucggauguuuacagc

SEQ ID NO: 333 - RNA, 22 nt, Homo sapiens
ccuauucuugauuacuuguuuc

SEQ ID NO: 334 - RNA, 22 nt, Homo sapiens
uuaucagaaucuccagggguac

SEQ ID NO: 335 - RNA, 23 nt, Homo sapiens
ucccccaggugugauucugauuu

SEQ ID NO: 336 - RNA, 24 nt, Homo sapiens
aauccuuggaaccuaggugugagu

SEQ ID NO: 337 - RNA, 22 nt, Homo sapiens
aacacaccuauucaaggauuca

SEQ ID NO: 338 - RNA, 22 nt, Homo sapiens
cggguggaucacgaugcaauuu

SEQ ID NO: 339 - RNA, 22 nt, Homo sapiens
aauugcacgguauccaucugua

SEQ ID NO: 340 - RNA, 22 nt, Homo sapiens
uaaugccccuaaaaauccuuau

SEQ ID NO: 341 - RNA, 22 nt, Homo sapiens
agggacuuucaggggcagcugu

SEQ ID NO: 342 - RNA, 22 nt, Homo sapiens
acuuuaacauggaagugcuuuc

SEQ ID NO: 343 - RNA, 23 nt, Homo sapiens
uaagugcuuccauguuuuaguag

SEQ ID NO: 344 - RNA, 22 nt, Homo sapiens
uuuaacauggggguaccugcug

SEQ ID NO: 345 - RNA, 23 nt, Homo sapiens
uaagugcuuccauguuucagugg

SEQ ID NO: 346 - RNA, 22 nt, Homo sapiens
acuuuaacauggaggcacuugc

SEQ ID NO: 347 - RNA, 23 nt, Homo sapiens
uaagugcuuccauguuugagugu

SEQ ID NO: 348 - RNA, 22 nt, Homo sapiens
acuguugcuaauaugcaacucu

SEQ ID NO: 349 - RNA, 22 nt, Homo sapiens
aauugcacuuuagcaaugguga

SEQ ID NO: 350 - RNA, 21 nt, Homo sapiens
aacauagaggaaauuccacgu

SEQ ID NO: 351 - RNA, 22 nt, Homo sapiens
agaucgaccguguuauauucgc

SEQ ID NO: 352 - RNA, 21 nt, Homo sapiens
aauaauacaugguugaucuuu

SEQ ID NO: 353 - RNA, 22 nt, Homo sapiens
gccugcugggguggaaccuggu

SEQ ID NO: 354 - RNA, 20 nt, Homo sapiens
acucaaacugugggggcacu

SEQ ID NO: 355 - RNA, 23 nt, Homo sapiens
aagugccgccaucuuuugagugu

SEQ ID NO: 356 - RNA, 23 nt, Homo sapiens
aaagugcugcgacauuugagcgu

SEQ ID NO: 357 - RNA, 22 nt, Homo sapiens
acucaaaaugggggcgcuuucc

SEQ ID NO: 358 - RNA, 23 nt, Homo sapiens
gaagugcuucgauuuuggggugu

SEQ ID NO: 359 - RNA, 22 nt, Homo sapiens
uuauaauacaaccugauaagug

SEQ ID NO: 360 - RNA, 22 nt, Homo sapiens
cuuaucagauuguauuguaauu

SEQ ID NO: 361 - RNA, 22 nt, Homo sapiens
uuuguucguucggcucgcguga

SEQ ID NO: 362 - RNA, 22 nt, Homo sapiens
guagauucuccuucuaugagua

SEQ ID NO: 363 - RNA, 21 nt, Homo sapiens
aucauagaggaaaauccacgu

SEQ ID NO: 364 - RNA, 22 nt, Homo sapiens
agagguugcccuuggugaauuc

SEQ ID NO: 365 - RNA, 22 nt, Homo sapiens
aucacacaaaggcaacuuuugu

SEQ ID NO: 366 - RNA, 22 nt, Homo sapiens
cuccugacuccagguccugugu

SEQ ID NO: 367 - RNA, 21 nt, Homo sapiens
acuggacuuggagucagaagg

SEQ ID NO: 368 - RNA, 21 nt, Homo sapiens
ugguagacuauggaacguagg

SEQ ID NO: 369 - RNA, 22 nt, Homo sapiens
uauguaacaugguccacuaacu

SEQ ID NO: 370 - RNA, 22 nt, Homo sapiens
ugguugaccauagaacaugcgc

SEQ ID NO: 371 - RNA, 22 nt, Homo sapiens
uauguaauaugguccacaucuu

SEQ ID NO: 372 - RNA, 22 nt, Homo sapiens
uauacaagggcaagcucucugu

SEQ ID NO: 373 - RNA, 22 nt, Homo sapiens
gaaguuguucgugguggauucg

SEQ ID NO: 374 - RNA, 22 nt, Homo sapiens
agaucagaaggugauuguggcu

SEQ ID NO: 375 - RNA, 22 nt, Homo sapiens
uuauaaagcaaugagacugauu

SEQ ID NO: 376 - RNA, 22 nt, Homo sapiens
uccgucucaguuacuuuauagc

SEQ ID NO: 377 - RNA, 22 nt, Homo sapiens
ucucugggccugugucuuaggc

SEQ ID NO: 378 - RNA, 23 nt, Homo sapiens
gcaaagcacacggccugcagaga

SEQ ID NO: 379 - RNA, 22 nt, Homo sapiens
cuggcccucucugcccuuccgu

SEQ ID NO: 380 - RNA, 21 nt, Homo sapiens
aggggugcuaucugugauuga

SEQ ID NO: 381 - RNA, 23 nt, Homo sapiens
ucucacacagaaaucgcacccgu

SEQ ID NO: 382 - RNA, 21 nt, Homo sapiens
gaacggcuucauacaggaguu

SEQ ID NO: 383 - RNA, 22 nt, Homo sapiens
cuccuauaugaugccuuucuuc

SEQ ID NO: 384 - RNA, 22 nt, Homo sapiens
aggugguccguggcgcguucgc

SEQ ID NO: 385 - RNA, 21 nt, Homo sapiens
cacauuacacggucgaccucu

SEQ ID NO: 386 - RNA, 20 nt, Homo sapiens
ccucugggcccuuccuccag

SEQ ID NO: 387 - RNA, 21 nt, Homo sapiens
ucgaggagcucacagucuagu

SEQ ID NO: 388 - RNA, 21 nt, Homo sapiens
cuagacugaagcuccuugagg

SEQ ID NO: 389 - RNA, 23 nt, Homo sapiens
uauggcuuuucauuccuauguga

SEQ ID NO: 390 - RNA, 22 nt, Homo sapiens
auguagggcuaaaagccauggg

SEQ ID NO: 391 - RNA, 22 nt, Homo sapiens
aaguucuguuauacacucaggc

SEQ ID NO: 392 - RNA, 22 nt, Homo sapiens
ucagugcaucacagaacuuugu

SEQ ID NO: 393 - RNA, 22 nt, Homo sapiens
cuagguauggucccagggaucc

SEQ ID NO: 394 - RNA, 21 nt, Homo sapiens
gccccugggccuauccuagaa

SEQ ID NO: 395 - RNA, 23 nt, Homo sapiens
cgcauccccuagggcauuggugu

SEQ ID NO: 396 - RNA, 20 nt, Homo sapiens
acugccccaggugcugcugg

SEQ ID NO: 397 - RNA, 22 nt, Homo sapiens
aacaauauccuggugcugagug

SEQ ID NO: 398 - RNA, 22 nt, Homo sapiens
uccagcaucagugauuuuguug

SEQ ID NO: 399 - RNA, 23 nt, Homo sapiens
ucccuguccuccaggagcucacg

SEQ ID NO: 400 - RNA, 23 nt, Homo sapiens
ugagcgccucgacgacagagccg

SEQ ID NO: 401 - RNA, 23 nt, Homo sapiens
ucaagagcaauaacgaaaaaugu

SEQ ID NO: 402 - RNA, 22 nt, Homo sapiens
uuuuucauuauugcuccugacc

SEQ ID NO: 403 - RNA, 22 nt, Homo sapiens
uuugguccccuucaaccagcua

SEQ ID NO: 404 - RNA, 23 nt, Homo sapiens
ccuaguagguguccaguaagugu

SEQ ID NO: 405 - RNA, 22 nt, Homo sapiens
gcugacuccuaguccagggcuc

SEQ ID NO: 406 - RNA, 23 nt, Homo sapiens
ugucugcccgcaugccugccucu

SEQ ID NO: 407 - RNA, 20 nt, Homo sapiens
auuccuagaaauuguucaua

SEQ ID NO: 408 - RNA, 22 nt, Homo sapiens
uagguaguuuccuguuguuggg

SEQ ID NO: 409 - RNA, 22 nt, Homo sapiens
ucgacagcacgacacugccuuc

SEQ ID NO: 410 - RNA, 22 nt, Homo sapiens
acuggacuuagggucagaaggc

SEQ ID NO: 411 - RNA, 23 nt, Homo sapiens
ugaggggcagagagcgagacuuu

SEQ ID NO: 412 - RNA, 23 nt, Homo sapiens
agcucggucugaggccccucagu

SEQ ID NO: 413 - RNA, 22 nt, Homo sapiens
cagcagcaauucauguuuugaa

SEQ ID NO: 414 - RNA, 21 nt, Homo sapiens
caaaacgugaggcgcugcuau

SEQ ID NO: 415 - RNA, 23 nt, Homo sapiens
aaugacacgaucacucccguuga

SEQ ID NO: 416 - RNA, 22 nt, Homo sapiens
aucgggaaugucguguccgccc

SEQ ID NO: 417 - RNA, 23 nt, Homo sapiens
uaaggugcaucuagugcaguuag

SEQ ID NO: 418 - RNA, 22 nt, Homo sapiens
ugcccuaaaugccccuucuggc

SEQ ID NO: 419 - RNA, 23 nt, Homo sapiens
caaagugcucauagugcagguag

SEQ ID NO: 420 - RNA, 22 nt, Homo sapiens
acuguaguaugggcacuuccag

SEQ ID NO: 421 - RNA, 22 nt, Homo sapiens
uugcauauguaggaugucccau

SEQ ID NO: 422 - RNA, 22 nt, Homo sapiens
uaauacugucugguaaaaccgu

SEQ ID NO: 423 - RNA, 22 nt, Homo sapiens
uggcaguguauuguuagcuggu

SEQ ID NO: 424 - RNA, 22 nt, Homo sapiens
uuuugcgauguguuccuaauau

SEQ ID NO: 425 - RNA, 21 nt, Homo sapiens
ugucuugcaggccgucaugca

SEQ ID NO: 426 - RNA, 22 nt, Homo sapiens
caggucgucuugcagggcuucu

SEQ ID NO: 427 - RNA, 22 nt, Homo sapiens
aucaugaugggcuccucggugu

SEQ ID NO: 428 - RNA, 22 nt, Homo sapiens
aacacaccugguuaaccucuuu

SEQ ID NO: 429 - RNA, 22 nt, Homo sapiens
aaaccguuaccauuacugaguu

SEQ ID NO: 430 - RNA, 22 nt, Homo sapiens
aacuguuugcagaggaaacuga

SEQ ID NO: 431 - RNA, 22 nt, Homo sapiens
cucaucugcaaagaaguaagug

SEQ ID NO: 432 - RNA, 23 nt, Homo sapiens
agguuacccgagcaacuuugcau

SEQ ID NO: 433 - RNA, 22 nt, Homo sapiens
gaauguugcucggugaaccccu

SEQ ID NO: 434 - RNA, 23 nt, Homo sapiens
acuucaccugguccacuagccgu

SEQ ID NO: 435 - RNA, 21 nt, Homo sapiens
aauauaacacagauggccugu

SEQ ID NO: 436 - RNA, 22 nt, Homo sapiens
aucauagaggaaaauccauguu

SEQ ID NO: 437 - RNA, 22 nt, Homo sapiens
aagacgggaggaaagaagggag

SEQ ID NO: 438 - RNA, 21 nt, Homo sapiens
ucacuccucuccucccgucuu

SEQ ID NO: 439 - RNA, 22 nt, Homo sapiens
ucaggcucaguccccucccgau

SEQ ID NO: 440 - RNA, 22 nt, Homo sapiens
agaggcuggccgugaugaauuc

SEQ ID NO: 441 - RNA, 22 nt, Homo sapiens
gucauacacggcucuccucucu

SEQ ID NO: 442 - RNA, 22 nt, Homo sapiens
uccuguacugagcugccccgag

SEQ ID NO: 443 - RNA, 21 nt, Homo sapiens
cggggcagcucaguacaggau

SEQ ID NO: 444 - RNA, 22 nt, Homo sapiens
aaucauacagggacauccaguu

SEQ ID NO: 445 - RNA, 21 nt, Homo sapiens
cccagauaauggcacucucaa

SEQ ID NO: 446 - RNA, 21 nt, Homo sapiens
uugaaaggcuauuucuugguc

SEQ ID NO: 447 - RNA, 22 nt, Homo sapiens
gugacaucacauauacggcagc

SEQ ID NO: 448 - RNA, 20 nt, Homo sapiens
ccauggaucuccaggugggu

SEQ ID NO: 449 - RNA, 22 nt, Homo sapiens
caaccuggaggacuccaugcug

SEQ ID NO: 450 - RNA, 22 nt, Homo sapiens
aguggggaacccuuccaugagg

SEQ ID NO: 451 - RNA, 22 nt, Homo sapiens
cuuaugcaagauucccuucuac

SEQ ID NO: 452 - RNA, 21 nt, Homo sapiens
gugucuuuugcucugcaguca

SEQ ID NO: 453 - RNA, 22 nt, Homo sapiens
ugagaacugaauuccauaggcu

SEQ ID NO: 454 - RNA, 22 nt, Homo sapiens
ugcccuguggacucaguucugg

SEQ ID NO: 455 - RNA, 22 nt, Homo sapiens
uuccuaugcauauacuucuuug

SEQ ID NO: 456 - RNA, 20 nt, Homo sapiens
agagguauagggcaugggaa

SEQ ID NO: 457 - RNA, 23 nt, Homo sapiens
aggaccugcgggacaagauucuu

SEQ ID NO: 458 - RNA, 22 nt, Homo sapiens
uuguacaugguaggcuuucauu

SEQ ID NO: 459 - RNA, 22 nt, Homo sapiens
ugaaggucuacugugugccagg

SEQ ID NO: 460 - RNA, 23 nt, Homo sapiens
ucuuggaguaggucauugggugg

SEQ ID NO: 461 - RNA, 21 nt, Homo sapiens
cuggauggcuccuccaugucu

SEQ ID NO: 462 - RNA, 22 nt, Homo sapiens
ugaaacauacacgggaaaccuc

SEQ ID NO: 463 - RNA, 22 nt, Homo sapiens
aaacaaacauggugcacuucuu

SEQ ID NO: 464 - RNA, 22 nt, Homo sapiens
ugaguauuacauggccaaucuc

SEQ ID NO: 465 - RNA, 22 nt, Homo sapiens
cgggguuuugagggcgagauga

SEQ ID NO: 466 - RNA, 22 nt, Homo sapiens
aacuggcccucaaagucccgcu

SEQ ID NO: 467 - RNA, 21 nt, Homo sapiens
cagcagcacacugugguuugu

SEQ ID NO: 468 - RNA, 22 nt, Homo sapiens
caaaccacacugugguguuaga

SEQ ID NO: 469 - RNA, 23 nt, Homo sapiens
aacauucauuguugucggugggu

SEQ ID NO: 470 - RNA, 23 nt, Homo sapiens
cacucagccuugagggcacuuuc

SEQ ID NO: 471 - RNA, 22 nt, Homo sapiens
aagugcugucauagcugagguc

SEQ ID NO: 472 - RNA, 23 nt, Homo sapiens
uuucaagccagggggcguuuuuc

SEQ ID NO: 473 - RNA, 21 nt, Homo sapiens
aaagugcuuccuuuuugaggg

SEQ ID NO: 474 - RNA, 24 nt, Homo sapiens
uucuccaaaagaaagcacuuucug

SEQ ID NO: 475 - RNA, 22 nt, Homo sapiens
gagugccuucuuuuggagcguu

SEQ ID NO: 476 - RNA, 22 nt, Homo sapiens
uucuccaaaagggagcacuuuc

SEQ ID NO: 477 - RNA, 22 nt, Homo sapiens
aagugccuccuuuuagaguguu

SEQ ID NO: 478 - RNA, 22 nt, Homo sapiens
aagugcuuccuuuuagaggguu

SEQ ID NO: 479 - RNA, 22 nt, Homo sapiens
cucuagagggaagcgcuuucug

SEQ ID NO: 480 - RNA, 22 nt, Homo sapiens
aaagugcaucuuuuuagaggau

SEQ ID NO: 481 - RNA, 21 nt, Homo sapiens
cuccagagggaaguacuuucu

SEQ ID NO: 482 - RNA, 22 nt, Homo sapiens
aaagugcuucccuuuggacugu

SEQ ID NO: 483 - RNA, 23 nt, Homo sapiens
cucuugagggaagcacuuucugu

SEQ ID NO: 484 - RNA, 22 nt, Homo sapiens
gaaagugcuuccuuuuagaggc

SEQ ID NO: 485 - RNA, 22 nt, Homo sapiens
cucuagagggaagcgcuuucug

SEQ ID NO: 486 - RNA, 22 nt, Homo sapiens
aaagugcauccuuuuagagguu

SEQ ID NO: 487 - RNA, 21 nt, Homo sapiens
cuccagagggaugcacuuucu

SEQ ID NO: 488 - RNA, 22 nt, Homo sapiens
gaaggcgcuucccuuuagagcg

SEQ ID NO: 489 - RNA, 22 nt, Homo sapiens
cucuagagggaagcgcuuucug

SEQ ID NO: 490 - RNA, 23 nt, Homo sapiens
gaacgcgcuucccuauagagggu

SEQ ID NO: 491 - RNA, 22 nt, Homo sapiens
cucuagagggaagcacuuucuc

SEQ ID NO: 492 - RNA, 21 nt, Homo sapiens
gaaagcgcuucucuuuagagg

SEQ ID NO: 493 - RNA, 21 nt, Homo sapiens
aaagugcuuccuuuuagaggg

SEQ ID NO: 494 - RNA, 22 nt, Homo sapiens
caaagcgcuccccuuuagaggu

SEQ ID NO: 495 - RNA, 22 nt, Homo sapiens
cucuagagggaagcacuuucug

SEQ ID NO: 496 - RNA, 22 nt, Homo sapiens
cucuagagggaagcacuuucug

SEQ ID NO: 497 - RNA, 22 nt, Homo sapiens
aaagugcuuccuuuuagagggu

SEQ ID NO: 498 - RNA, 23 nt, Homo sapiens
ucucuggagggaagcacuuucug

SEQ ID NO: 499 - RNA, 23 nt, Homo sapiens
caaagcgcuucucuuuagagugu

SEQ ID NO: 500 - RNA, 22 nt, Homo sapiens
cuacaaagggaagcacuuucuc

SEQ ID NO: 501 - RNA, 21 nt, Homo sapiens
gaaggcgcuucccuuuggagu

SEQ ID NO: 502 - RNA, 22 nt, Homo sapiens
ccucuagauggaagcacugucu

SEQ ID NO: 503 - RNA, 22 nt, Homo sapiens
aucgugcaucccuuuagagugu

SEQ ID NO: 504 - RNA, 22 nt, Homo sapiens
caaagugccucccuuuagagug

SEQ ID NO: 505 - RNA, 22 nt, Homo sapiens
aacgcacuucccuuuagagugu

SEQ ID NO: 506 - RNA, 20 nt, Homo sapiens
cuacaaagggaagcccuuuc

SEQ ID NO: 507 - RNA, 22 nt, Homo sapiens
aaagugcuucucuuuggugggu

SEQ ID NO: 508 - RNA, 22 nt, Homo sapiens
ucgugcaucccuuuagaguguu

SEQ ID NO: 509 - RNA, 24 nt, Homo sapiens
acaaagugcuucccuuuagagugu

SEQ ID NO: 510 - RNA, 22 nt, Homo sapiens
aucuggagguaagaagcacuuu

SEQ ID NO: 511 - RNA, 18 nt, Homo sapiens
ugcuuccuuucagagggu

SEQ ID NO: 512 - RNA, 22 nt, Homo sapiens
cucuagagggaagcgcuuucug

SEQ ID NO: 513 - RNA, 21 nt, Homo sapiens
aaagcgcuucccuucagagug

SEQ ID NO: 514 - RNA, 20 nt, Homo sapiens
cugcaaagggaagcccuuuc

SEQ ID NO: 515 - RNA, 22 nt, Homo sapiens
gaaagcgcuucccuuugcugga

SEQ ID NO: 516 - RNA, 22 nt, Homo sapiens
cucuagagggaagcacuuucug

SEQ ID NO: 517 - RNA, 21 nt, Homo sapiens
caaagcgcuucccuuuggagc

SEQ ID NO: 518 - RNA, 22 nt, Homo sapiens
aucgugcauccuuuuagagugu

SEQ ID NO: 519 - RNA, 22 nt, Homo sapiens
acaaagugcuucccuuuagagu

SEQ ID NO: 520 - RNA, 22 nt, Homo sapiens
cucuagagggaagcgcuuucug

SEQ ID NO: 521 - RNA, 22 nt, Homo sapiens
aaaaugguucccuuuagagugu

SEQ ID NO: 522 - RNA, 22 nt, Homo sapiens
cucuagagggaagcgcuuucug

SEQ ID NO: 523 - RNA, 22 nt, Homo sapiens
aaagugcauccuuuuagagugu

SEQ ID NO: 524 - RNA, 20 nt, Homo sapiens
cugcaaagggaagcccuuuc

SEQ ID NO: 525 - RNA, 23 nt, Homo sapiens
uucucgaggaaagaagcacuuuc

SEQ ID NO: 526 - RNA, 18 nt, Homo sapiens
ugcuuccuuucagagggu

SEQ ID NO: 527 - RNA, 21 nt, Homo sapiens
uuaagacuugcagugauguuu

SEQ ID NO: 528 - RNA, 22 nt, Homo sapiens
aacaucacagcaagucugugcu

SEQ ID NO: 529 - RNA, 23 nt, Homo sapiens
uaauccuugcuaccugggugaga

SEQ ID NO: 530 - RNA, 22 nt, Homo sapiens
augcaccugggcaaggauucug

SEQ ID NO: 531 - RNA, 22 nt, Homo sapiens
aauccuuugucccugggugaga

SEQ ID NO: 532 - RNA, 22 nt, Homo sapiens
aaugcacccgggcaaggauucu

SEQ ID NO: 533 - RNA, 21 nt, Homo sapiens
auccuugcuaucugggugcua

SEQ ID NO: 534 - RNA, 22 nt, Homo sapiens
aaugcaccugggcaaggauuca

SEQ ID NO: 535 - RNA, 23 nt, Homo sapiens
uagcagcgggaacaguucugcag

SEQ ID NO: 536 - RNA, 22 nt, Homo sapiens
agacccuggucugcacucuauc

SEQ ID NO: 537 - RNA, 22 nt, Homo sapiens
gggagccaggaaguauugaugu

SEQ ID NO: 538 - RNA, 22 nt, Homo sapiens
cgucaacacuugcugguuuccu

SEQ ID NO: 539 - RNA, 18 nt, Homo sapiens
uucacagggaggugucau

SEQ ID NO: 540 - RNA, 23 nt, Homo sapiens
uaaauuucaccuuucugagaagg

SEQ ID NO: 541 - RNA, 21 nt, Homo sapiens
uaaggcacccuucugaguaga

SEQ ID NO: 542 - RNA, 21 nt, Homo sapiens
uuuugcaccuuuuggagugaa

SEQ ID NO: 543 - RNA, 23 nt, Homo sapiens
uacuccagagggcgucacucaug

SEQ ID NO: 544 - RNA, 23 nt, Homo sapiens
ugauuguagccuuuuggaguaga

SEQ ID NO: 545 - RNA, 21 nt, Homo sapiens
uacugcagacaguggcaauca

SEQ ID NO: 546 - RNA, 22 nt, Homo sapiens
ugauugguacgucuguggguag

SEQ ID NO: 547 - RNA, 22 nt, Homo sapiens
uacucaggagaguggcaaucac

SEQ ID NO: 548 - RNA, 21 nt, Homo sapiens
auugacacuucugugaguaga

SEQ ID NO: 549 - RNA, 22 nt, Homo sapiens
caugccuugaguguaggaccgu

SEQ ID NO: 550 - RNA, 22 nt, Homo sapiens
ccucccacacccaaggcuugca

SEQ ID NO: 551 - RNA, 22 nt, Homo sapiens
uaugugccuuuggacuacaucg

SEQ ID NO: 552 - RNA, 21 nt, Homo sapiens
gcaguccaugggcauauacac

SEQ ID NO: 553 - RNA, 22 nt, Homo sapiens
ggagaaauuauccuuggugugu

SEQ ID NO: 554 - RNA, 22 nt, Homo sapiens
auucugcauuuuuagcaaguuc

SEQ ID NO: 555 - RNA, 22 nt, Homo sapiens
ucaguaaauguuuauuagauga

SEQ ID NO: 556 - RNA, 22 nt, Homo sapiens
ucagcaaacauuuauugugugc

SEQ ID NO: 557 - RNA, 22 nt, Homo sapiens
aaucguacagggucauccacuu

SEQ ID NO: 558 - RNA, 21 nt, Homo sapiens
gcgacccacucuugguuucca

SEQ ID NO: 559 - RNA, 21 nt, Homo sapiens
aacaggugacugguuagacaa

SEQ ID NO: 560 - RNA, 21 nt, Homo sapiens
aaaacggugagauuuuguuuu

SEQ ID NO: 561 - RNA, 21 nt, Homo sapiens
gcuaguccugacucagccagu

SEQ ID NO: 562 - RNA, 22 nt, Homo sapiens
agggacgggacgcggugcagug

SEQ ID NO: 563 - RNA, 22 nt, Homo sapiens
uauugcacucgucccggccucc

SEQ ID NO: 564 - RNA, 21 nt, Homo sapiens
aggguaagcugaaccucugau

SEQ ID NO: 565 - RNA, 22 nt, Homo sapiens
gaugagcucauuguaauaugag

SEQ ID NO: 566 - RNA, 22 nt, Homo sapiens
auauuaccauuagcucaucuuu

SEQ ID NO: 567 - RNA, 23 nt, Homo sapiens
guuugcacgggugggccuugucu

SEQ ID NO: 568 - RNA, 19 nt, Homo sapiens
ugagcugcuguaccaaaau

SEQ ID NO: 569 - RNA, 21 nt, Homo sapiens
uaaaguaaauaugcaccaaaa

SEQ ID NO: 570 - RNA, 22 nt, Homo sapiens
caaaguuuaagauccuugaagu

SEQ ID NO: 571 - RNA, 20 nt, Homo sapiens
aaaguagcuguaccauuugc

SEQ ID NO: 572 - RNA, 19 nt, Homo sapiens
agguugacauacguuuccc

SEQ ID NO: 573 - RNA, 19 nt, Homo sapiens
aggcacggugucagcaggc

SEQ ID NO: 574 - RNA, 19 nt, Homo sapiens
gggcgccugugaucccaac

SEQ ID NO: 575 - RNA, 23 nt, Homo sapiens
aguauguucuuccaggacagaac

SEQ ID NO: 576 - RNA, 20 nt, Homo sapiens
auguauaaauguauacacac

SEQ ID NO: 577 - RNA, 22 nt, Homo sapiens
gaaaucaagcgugggugagacc

SEQ ID NO: 578 - RNA, 21 nt, Homo sapiens
gcgacccauacuugguuucag

SEQ ID NO: 579 - RNA, 21 nt, Homo sapiens
aguuaaugaauccuggaaagu

SEQ ID NO: 580 - RNA, 22 nt, Homo sapiens
cgaaaacagcaauuaccuuugc

SEQ ID NO: 581 - RNA, 21 nt, Homo sapiens
ugaguuggccaucugagugag

SEQ ID NO: 582 - RNA, 20 nt, Homo sapiens
guccgcucggcgguggccca

SEQ ID NO: 583 - RNA, 24 nt, Homo sapiens
cugaagugauguguaacugaucag

SEQ ID NO: 584 - RNA, 23 nt, Homo sapiens
ugagugugugugugugagugugu

SEQ ID NO: 585 - RNA, 22 nt, Homo sapiens
cacgcucaugcacacacccaca

SEQ ID NO: 586 - RNA, 19 nt, Homo sapiens
gagccaguuggacaggagc

SEQ ID NO: 587 - RNA, 22 nt, Homo sapiens
auucuaauuucuccacgucuuu

SEQ ID NO: 588 - RNA, 22 nt, Homo sapiens
aagauguggaaaaauuggaauc

SEQ ID NO: 589 - RNA, 21 nt, Homo sapiens
uagauaaaauauugguaccug

SEQ ID NO: 590 - RNA, 21 nt, Homo sapiens
cuucuugugcucuaggauugu

SEQ ID NO: 591 - RNA, 23 nt, Homo sapiens
uucauuugguauaaaccgcgauu

SEQ ID NO: 592 - RNA, 22 nt, Homo sapiens
uugagaaugaugaaucauuagg

SEQ ID NO: 593 - RNA, 21 nt, Homo sapiens
ucuuguguucucuagaucagu

SEQ ID NO: 594 - RNA, 23 nt, Homo sapiens
uuacaguuguucaaccaguuacu

SEQ ID NO: 595 - RNA, 22 nt, Homo sapiens
uaacugguugaacaacugaacc

SEQ ID NO: 596 - RNA, 21 nt, Homo sapiens
caaagaggaaggucccauuac

SEQ ID NO: 597 - RNA, 22 nt, Homo sapiens
uuaugguuugccugggacugag

SEQ ID NO: 598 - RNA, 19 nt, Homo sapiens
ugggcguaucuguaugcua

SEQ ID NO: 599 - RNA, 22 nt, Homo sapiens
caaaacuggcaauuacuuuugc

SEQ ID NO: 600 - RNA, 22 nt, Homo sapiens
uaugcauuguauuuuuaggucc

SEQ ID NO: 601 - RNA, 21 nt, Homo sapiens
uuuccauaggugaugagucac

SEQ ID NO: 602 - RNA, 22 nt, Homo sapiens
aaaaguaauugugguuuuggcc

SEQ ID NO: 603 - RNA, 22 nt, Homo sapiens
caagaaccucaguugcuuuugu

SEQ ID NO: 604 - RNA, 21 nt, Homo sapiens
uuggccacaauggguuagaac

SEQ ID NO: 605 - RNA, 22 nt, Homo sapiens
ugagaaccacgucugcucugag

SEQ ID NO: 606 - RNA, 24 nt, Homo sapiens
ucagaacaaaugccgguucccaga

SEQ ID NO: 607 - RNA, 23 nt, Homo sapiens
agugccugagggaguaagagccc

SEQ ID NO: 608 - RNA, 22 nt, Homo sapiens
ugucuuacucccucaggcacau

SEQ ID NO: 609 - RNA, 22 nt, Homo sapiens
gagcuuauucauaaaagugcag

SEQ ID NO: 610 - RNA, 21 nt, Homo sapiens
uaauuuuauguauaagcuagu

SEQ ID NO: 611 - RNA, 20 nt, Homo sapiens
agaccauggguucucauugu

SEQ ID NO: 612 - RNA, 22 nt, Homo sapiens
uugugucaauaugcgaugaugu

SEQ ID NO: 613 - RNA, 25 nt, Homo sapiens
aggcaccagccaggcauugcucagc

SEQ ID NO: 614 - RNA, 19 nt, Homo sapiens
ugucucugcugggguuucu

SEQ ID NO: 615 - RNA, 21 nt, Homo sapiens
gaagugugccguggugugucu

SEQ ID NO: 616 - RNA, 21 nt, Homo sapiens
aagccugcccggcuccucggg

SEQ ID NO: 617 - RNA, 22 nt, Homo sapiens
ugugucacucgaugaccacugu

SEQ ID NO: 618 - RNA, 22 nt, Homo sapiens
uacgucaucguugucaucguca

SEQ ID NO: 619 - RNA, 20 nt, Homo sapiens
guugugucaguuuaucaaac

SEQ ID NO: 620 - RNA, 22 nt, Homo sapiens
aaaaguaauugcgaguuuuacc

SEQ ID NO: 621 - RNA, 23 nt, Homo sapiens
acuuacagacaagagccuugcuc

SEQ ID NO: 622 - RNA, 22 nt, Homo sapiens
uggucuaggauuguuggaggag

SEQ ID NO: 623 - RNA, 23 nt, Homo sapiens
gacacgggcgacagcugcggccc

SEQ ID NO: 624 - RNA, 22 nt, Homo sapiens
cacacacugcaauuacuuuugc

SEQ ID NO: 625 - RNA, 19 nt, Homo sapiens
aggcugcggaauucaggac

SEQ ID NO: 626 - RNA, 23 nt, Homo sapiens
uaaaucccauggugccuucuccu

SEQ ID NO: 627 - RNA, 21 nt, Homo sapiens
aaacuacugaaaaucaaagau

SEQ ID NO: 628 - RNA, 21 nt, Homo sapiens
guucaaauccagaucuauaac

SEQ ID NO: 629 - RNA, 25 nt, Homo sapiens
aggggugguguugggacagcuccgu

SEQ ID NO: 630 - RNA, 20 nt, Homo sapiens
aggguguuucucucaucucu

SEQ ID NO: 631 - RNA, 21 nt, Homo sapiens
ugagcuaaaugugugcuggga

SEQ ID NO: 632 - RNA, 23 nt, Homo sapiens
gcgaggaccccucggggucugac

SEQ ID NO: 633 - RNA, 25 nt, Homo sapiens
gcugggcagggcuucugagcuccuu

SEQ ID NO: 634 - RNA, 20 nt, Homo sapiens
aggaauguuccuucuuugcc

SEQ ID NO: 635 - RNA, 23 nt, Homo sapiens
gaacgccuguucuugccaggugg

SEQ ID NO: 636 - RNA, 22 nt, Homo sapiens
ggggguccccggugcucggauc

SEQ ID NO: 637 - RNA, 22 nt, Homo sapiens
uccgagccugggucucccucuu

SEQ ID NO: 638 - RNA, 22 nt, Homo sapiens
acucaaaacccuucagugacuu

SEQ ID NO: 639 - RNA, 22 nt, Homo sapiens
agucauuggaggguuugagcag

SEQ ID NO: 640 - RNA, 22 nt, Homo sapiens
aaaaguaauugcgguuuuugcc

SEQ ID NO: 641 - RNA, 22 nt, Homo sapiens
caaaaaucucaauuacuuuugc

SEQ ID NO: 642 - RNA, 22 nt, Homo sapiens
agacuucccauuugaagguggc

SEQ ID NO: 643 - RNA, 23 nt, Homo sapiens
aaacucuacuuguccuucugagu

SEQ ID NO: 644 - RNA, 24 nt, Homo sapiens
gaccuggacauguuugugcccagu

SEQ ID NO: 645 - RNA, 20 nt, Homo sapiens
auggagauagauauagaaau

SEQ ID NO: 646 - RNA, 21 nt, Homo sapiens
ggcuagcaacagcgcuuaccu

SEQ ID NO: 647 - RNA, 21 nt, Homo sapiens
acagucugcugagguuggagc

SEQ ID NO: 648 - RNA, 23 nt, Homo sapiens
aucccuugcaggggcuguugggu

SEQ ID NO: 649 - RNA, 22 nt, Homo sapiens
uaguaccaguaccuuguguuca

SEQ ID NO: 650 - RNA, 21 nt, Homo sapiens
cacaagguauugguauuaccu

SEQ ID NO: 651 - RNA, 21 nt, Homo sapiens
agggggaaaguucuauagucc

SEQ ID NO: 652 - RNA, 22 nt, Homo sapiens
gacuauagaacuuucccccuca

SEQ ID NO: 653 - RNA, 19 nt, Homo sapiens
agcugucugaaaaugucuu

SEQ ID NO: 654 - RNA, 22 nt, Homo sapiens
gugagucucuaagaaaagagga

SEQ ID NO: 655 - RNA, 22 nt, Homo sapiens
augcugacauauuuacuagagg

SEQ ID NO: 656 - RNA, 21 nt, Homo sapiens
ucuaguaagaguggcagucga

SEQ ID NO: 657 - RNA, 21 nt, Homo sapiens
uggguuuacguugggagaacu

SEQ ID NO: 658 - RNA, 22 nt, Homo sapiens
guucucccaacguaagcccagc

SEQ ID NO: 659 - RNA, 22 nt, Homo sapiens
aguauucuguaccagggaaggu

SEQ ID NO: 660 - RNA, 21 nt, Homo sapiens
agaccuggcccagaccucagc

SEQ ID NO: 661 - RNA, 20 nt, Homo sapiens
gugcauugcuguugcauugc

SEQ ID NO: 662 - RNA, 22 nt, Homo sapiens
cagugccucggcagugcagccc

SEQ ID NO: 663 - RNA, 19 nt, Homo sapiens
gugucugcuuccuguggga

SEQ ID NO: 664 - RNA, 23 nt, Homo sapiens
cuaauaguaucuaccacaauaaa

SEQ ID NO: 665 - RNA, 22 nt, Homo sapiens
aaccagcaccccaacuuuggac

SEQ ID NO: 666 - RNA, 23 nt, Homo sapiens
acuugggcacugaaacaaugucc

SEQ ID NO: 667 - RNA, 23 nt, Homo sapiens
ugugcuugcucgucccgcccgca

SEQ ID NO: 668 - RNA, 24 nt, Homo sapiens
acugggggcuuucgggcucugcgu

SEQ ID NO: 669 - RNA, 25 nt, Homo sapiens
agggaucgcgggcggguggcggccu

SEQ ID NO: 670 - RNA, 23 nt, Homo sapiens
aucgcugcgguugcgagcgcugu

SEQ ID NO: 671 - RNA, 21 nt, Homo sapiens
augauccaggaaccugccucu

SEQ ID NO: 672 - RNA, 24 nt, Homo sapiens
aaagacauaggauagagucaccuc

SEQ ID NO: 673 - RNA, 22 nt, Homo sapiens
gucccucuccaaaugugucuug

SEQ ID NO: 674 - RNA, 22 nt, Homo sapiens
acuuguaugcuagcucagguag

SEQ ID NO: 675 - RNA, 19 nt, Homo sapiens
aguguggcuuucuuagagc

SEQ ID NO: 676 - RNA, 19 nt, Homo sapiens
ucuaggcugguacugcuga

SEQ ID NO: 677 - RNA, 19 nt, Homo sapiens
aagcagcugccucugaggc

SEQ ID NO: 678 - RNA, 21 nt, Homo sapiens
guggcugcacucacuuccuuc

SEQ ID NO: 679 - RNA, 19 nt, Homo sapiens
aagugugcagggcacuggu

SEQ ID NO: 680 - RNA, 22 nt, Homo sapiens
aaaccuguguuguucaagaguc

SEQ ID NO: 681 - RNA, 21 nt, Homo sapiens
aggaggcagcgcucucaggac

SEQ ID NO: 682 - RNA, 22 nt, Homo sapiens
uuuaggauaagcuugacuuuug

SEQ ID NO: 683 - RNA, 21 nt, Homo sapiens
aauggcgccacuaggguugug

SEQ ID NO: 684 - RNA, 22 nt, Homo sapiens
aaaaguaauugugguuuuugcc

SEQ ID NO: 685 - RNA, 22 nt, Homo sapiens
caaaaaccacaguuucuuuugc

SEQ ID NO: 686 - RNA, 24 nt, Homo sapiens
ugccugggucucuggccugcgcgu

SEQ ID NO: 687 - RNA, 21 nt, Homo sapiens
ucccacguuguggcccagcag

SEQ ID NO: 688 - RNA, 22 nt, Homo sapiens
aggcggggcgccgcgggaccgc

SEQ ID NO: 689 - RNA, 22 nt, Homo sapiens
aggcaguguauuguuagcuggc

SEQ ID NO: 690 - RNA, 22 nt, Homo sapiens
cagccacaacuacccugccacu

SEQ ID NO: 691 - RNA, 21 nt, Homo sapiens
guguugaaacaaucucuacug

SEQ ID NO: 692 - RNA, 21 nt, Homo sapiens
uaguagaccguauagcguacg

SEQ ID NO: 693 - RNA, 22 nt, Homo sapiens
uauguaacacgguccacuaacc

SEQ ID NO: 694 - RNA, 22 nt, Homo sapiens
uggugggccgcagaacaugugc

SEQ ID NO: 695 - RNA, 22 nt, Homo sapiens
uaugucugcugaccaucaccuu

SEQ ID NO: 696 - RNA, 22 nt, Homo sapiens
auaauacaugguuaaccucuuu

SEQ ID NO: 697 - RNA, 21 nt, Homo sapiens
aauauuauacagucaaccucu

SEQ ID NO: 698 - RNA, 21 nt, Homo sapiens
ugacaacuauggaugagcucu

SEQ ID NO: 699 - RNA, 23 nt, Homo sapiens
ggcagguucucacccucucuagg

SEQ ID NO: 700 - RNA, 25 nt, Homo sapiens
ggcggagggaaguagguccguuggu

SEQ ID NO: 701 - RNA, 22 nt, Homo sapiens
cuugguucagggagggucccca

SEQ ID NO: 702 - RNA, 22 nt, Homo sapiens
uacccauugcauaucggaguug

SEQ ID NO: 703 - RNA, 23 nt, Homo sapiens
aucaacagacauuaauugggcgc

SEQ ID NO: 704 - RNA, 23 nt, Homo sapiens
ucggggaucaucaugucacgaga

SEQ ID NO: 705 - RNA, 22 nt, Homo sapiens
ugugacagauugauaacugaaa

SEQ ID NO: 706 - RNA, 22 nt, Homo sapiens
uuugugaccugguccacuaacc

SEQ ID NO: 707 - RNA, 23 nt, Homo sapiens
caagucuuauuugagcaccuguu

SEQ ID NO: 708 - RNA, 23 nt, Homo sapiens
aggaagcccuggaggggcuggag

SEQ ID NO: 709 - RNA, 21 nt, Homo sapiens
uccgguucucagggcuccacc

SEQ ID NO: 710 - RNA, 23 nt, Homo sapiens
ugucacucggcucggcccacuac

SEQ ID NO: 711 - RNA, 23 nt, Homo sapiens
ugcaccaugguugucugagcaug

SEQ ID NO: 712 - RNA, 23 nt, Homo sapiens
ucugcucauaccccaugguuucu

SEQ ID NO: 713 - RNA, 19 nt, Homo sapiens
gugaggacucgggaggugg

SEQ ID NO: 714 - RNA, 21 nt, Homo sapiens
ccccaccuccucucuccucag

SEQ ID NO: 715 - RNA, 22 nt, Homo sapiens
aaaagcuggguugagagggcaa

SEQ ID NO: 716 - RNA, 20 nt, Homo sapiens
aaaagcuggguugagagggu

SEQ ID NO: 717 - RNA, 22 nt, Homo sapiens
uuagggcccuggcuccaucucc

SEQ ID NO: 718 - RNA, 21 nt, Homo sapiens
cuccguuugccuguuucgcug

SEQ ID NO: 719 - RNA, 22 nt, Homo sapiens
ucaaaacugaggggcauuuucu

SEQ ID NO: 720 - RNA, 22 nt, Homo sapiens
cuuggcaccuagcaagcacuca

SEQ ID NO: 721 - RNA, 24 nt, Homo sapiens
uugcagcugccugggagugacuuc

SEQ ID NO: 722 - RNA, 22 nt, Homo sapiens
acccuaucaauauugucucugc

SEQ ID NO: 723 - RNA, 23 nt, Homo sapiens
uagugcaauauugcuuauagggu

SEQ ID NO: 724 - RNA, 21 nt, Homo sapiens
agaggauacccuuuguauguu

SEQ ID NO: 725 - RNA, 25 nt, Homo sapiens
uaggcaguguauugcuagcggcugu

SEQ ID NO: 726 - RNA, 23 nt, Homo sapiens
uugcuaguugcacuccucucugu

SEQ ID NO: 727 - RNA, 22 nt, Homo sapiens
ucuacaaaggaaagcgcuuucu

SEQ ID NO: 728 - RNA, 22 nt, Homo sapiens
ugagaccucuggguucugagcu

SEQ ID NO: 729 - RNA, 23 nt, Homo sapiens
cugggaucuccggggucuugguu

SEQ ID NO: 730 - RNA, 22 nt, Homo sapiens
acuccagccccacagccucagc

SEQ ID NO: 731 - RNA, 22 nt, Homo sapiens
ggggcuggggccggggccgagc

SEQ ID NO: 732 - RNA, 23 nt, Homo sapiens
caguaacaaagauucauccuugu

SEQ ID NO: 733 - RNA, 20 nt, Homo sapiens
gucccugaguguauguggug

SEQ ID NO: 734 - RNA, 22 nt, Homo sapiens
uucauucggcuguccagaugua

SEQ ID NO: 735 - RNA, 21 nt, Homo sapiens
auuugugcuuggcucugucac

SEQ ID NO: 736 - RNA, 22 nt, Homo sapiens
gcagcagggugaaacugacaca

SEQ ID NO: 737 - RNA, 22 nt, Homo sapiens
gcaggugcucacuuguccuccu

SEQ ID NO: 738 - RNA, 22 nt, Homo sapiens
gcagagugcaaacaauuuugac

SEQ ID NO: 739 - RNA, 21 nt, Homo sapiens
uggaggagaaggaaggugaug

SEQ ID NO: 740 - RNA, 23 nt, Homo sapiens
uccaguaccacgugucagggcca

SEQ ID NO: 741 - RNA, 23 nt, Homo sapiens
uggugcggagagggcccacagug

SEQ ID NO: 742 - RNA, 20 nt, Homo sapiens
cuguaugcccucaccgcuca

SEQ ID NO: 743 - RNA, 24 nt, Homo sapiens
agcagaagcagggagguucuccca

SEQ ID NO: 744 - RNA, 22 nt, Homo sapiens
ugcaacgaaccugagccacuga

SEQ ID NO: 745 - RNA, 22 nt, Homo sapiens
uauacaagggcagacucucucu

SEQ ID NO: 746 - RNA, 23 nt, Homo sapiens
cgggucggaguuagcucaagcgg

SEQ ID NO: 747 - RNA, 21 nt, Homo sapiens
cgcgggugcuuacugacccuu

SEQ ID NO: 748 - RNA, 21 nt, Homo sapiens
cacuguguccuuucugcguag

SEQ ID NO: 749 - RNA, 21 nt, Homo sapiens
ccaccaccgugucugacacuu

SEQ ID NO: 750 - RNA, 22 nt, Homo sapiens
uuuugcaauauguuccugaaua

SEQ ID NO: 751 - RNA, 22 nt, Homo sapiens
uugggaucauuuugcauccaua

SEQ ID NO: 752 - RNA, 22 nt, Homo sapiens
cugcccuggcccgagggaccga

SEQ ID NO: 753 - RNA, 21 nt, Homo sapiens
uacuuggaaaggcaucaguug

SEQ ID NO: 754 - RNA, 22 nt, Homo sapiens
ugcaacuuaccugagucauuga

SEQ ID NO: 755 - RNA, 22 nt, Homo sapiens
acacagggcuguugugaagacu

SEQ ID NO: 756 - RNA, 21 nt, Homo sapiens
uacucaaaaagcugucaguca

SEQ ID NO: 757 - RNA, 22 nt, Homo sapiens
gacugacaccucuuugggugaa

SEQ ID NO: 758 - RNA, 22 nt, Homo sapiens
cacuggcuccuuucuggguaga

SEQ ID NO: 759 - RNA, 25 nt, Homo sapiens
aaaggauucugcugucggucccacu

SEQ ID NO: 760 - RNA, 22 nt, Homo sapiens
uggugggcacagaaucuggacu

SEQ ID NO: 761 - RNA, 21 nt, Homo sapiens
uuaauaucggacaaccauugu

SEQ ID NO: 762 - RNA, 22 nt, Homo sapiens
uauaccucaguuuuaucaggug

SEQ ID NO: 763 - RNA, 21 nt, Homo sapiens
ccuggaaacacugagguugug

SEQ ID NO: 764 - RNA, 22 nt, Homo sapiens
uggauuucuuugugaaucacca

SEQ ID NO: 765 - RNA, 22 nt, Homo sapiens
uggugguuuacaaaguaauuca

SEQ ID NO: 766 - RNA, 23 nt, Homo sapiens
aaggagcuuacaaucuagcuggg

SEQ ID NO: 767 - RNA, 22 nt, Homo sapiens
caacuagacugugagcuucuag

SEQ ID NO: 768 - RNA, 22 nt, Homo sapiens
gugugcggaaaugcuucugcua

SEQ ID NO: 769 - RNA, 21 nt, Homo sapiens
ugauauguuugauauuggguu

SEQ ID NO: 770 - RNA, 22 nt, Homo sapiens
ugcggggcuagggcuaacagca

SEQ ID NO: 771 - RNA, 22 nt, Homo sapiens
cuguugccacuaaccucaaccu

SEQ ID NO: 772 - RNA, 22 nt, Homo sapiens
uccauuacacuacccugccucu

SEQ ID NO: 773 - RNA, 22 nt, Homo sapiens
aggcagcgggguguaguggaua

SEQ ID NO: 774 - RNA, 20 nt, Homo sapiens
guagaggagauggcgcaggg

SEQ ID NO: 775 - RNA, 21 nt, Homo sapiens
uccucuucucccuccucccag

SEQ ID NO: 776 - RNA, 22 nt, Homo sapiens
gugaacgggcgccaucccgagg

SEQ ID NO: 777 - RNA, 20 nt, Homo sapiens
accaggaggcugaggccccu

SEQ ID NO: 778 - RNA, 21 nt, Homo sapiens
gcaggaacuugugagucuccu

SEQ ID NO: 779 - RNA, 22 nt, Homo sapiens
aaacauucgcggugcacuucuu

SEQ ID NO: 780 - RNA, 22 nt, Homo sapiens
auauaauacaaccugcuaagug

SEQ ID NO: 781 - RNA, 22 nt, Homo sapiens
cuuagcagguuguauuaucauu

SEQ ID NO: 782 - RNA, 20 nt, Homo sapiens
cggcucugggucugugggga

SEQ ID NO: 783 - RNA, 23 nt, Homo sapiens
cagugcaaugauauugucaaagc

SEQ ID NO: 784 - RNA, 22 nt, Homo sapiens
aaaucucugcaggcaaauguga

SEQ ID NO: 785 - RNA, 22 nt, Homo sapiens
auaagacgaacaaaagguuugu

SEQ ID NO: 786 - RNA, 20 nt, Homo sapiens
ggggagcuguggaagcagua

SEQ ID NO: 787 - RNA, 25 nt, Homo sapiens
cuagugagggacagaaccaggauuc

SEQ ID NO: 788 - RNA, 23 nt, Homo sapiens
gcagcagagaauaggacuacguc

SEQ ID NO: 789 - RNA, 20 nt, Homo sapiens
agagucuugugaugucuugc

SEQ ID NO: 790 - RNA, 22 nt, Homo sapiens
uacugcagacguggcaaucaug

SEQ ID NO: 791 - RNA, 22 nt, Homo sapiens
ugugcgcagggagaccucuccc

SEQ ID NO: 792 - RNA, 22 nt, Homo sapiens
ugucuacuacuggagacacugg

SEQ ID NO: 793 - RNA, 23 nt, Homo sapiens
ccaguuaccgcuuccgcuaccgc

SEQ ID NO: 794 - RNA, 22 nt, Homo sapiens
acaguagagggaggaaucgcag

SEQ ID NO: 795 - RNA, 22 nt, Homo sapiens
auccgcgcucugacucucugcc

SEQ ID NO: 796 - RNA, 22 nt, Homo sapiens
ugcccuuaaaggugaacccagu

SEQ ID NO: 797 - RNA, 24 nt, Homo sapiens
uggggagcugaggcucugggggug

SEQ ID NO: 798 - RNA, 21 nt, Homo sapiens
aaggcagggcccccgcucccc

SEQ ID NO: 799 - RNA, 23 nt, Homo sapiens
cacccggcugugugcacaugugc

SEQ ID NO: 800 - RNA, 22 nt, Homo sapiens
ucuucucuguuuuggccaugug

SEQ ID NO: 801 - RNA, 21 nt, Homo sapiens
cugacuguugccguccuccag

SEQ ID NO: 802 - RNA, 22 nt, Homo sapiens
aaauuauuguacaucggaugag

SEQ ID NO: 803 - RNA, 21 nt, Homo sapiens
auguaugugugcaugugcaug

SEQ ID NO: 804 - RNA, 21 nt, Homo sapiens
uugcucacuguucuucccuag

SEQ ID NO: 805 - RNA, 21 nt, Homo sapiens
aagcauucuuucauugguugg

SEQ ID NO: 806 - RNA, 22 nt, Homo sapiens
uuuccggcucgcgugggugugu

SEQ ID NO: 807 - RNA, 21 nt, Homo sapiens
ccgucgccgccacccgagccg

SEQ ID NO: 808 - RNA, 23 nt, Homo sapiens
gagggucuugggagggaugugac

SEQ ID NO: 809 - RNA, 27 nt, Homo sapiens
cacuguaggugauggugagagugggca

SEQ ID NO: 810 - RNA, 23 nt, Homo sapiens
ccugcagcgacuugauggcuucc

SEQ ID NO: 811 - RNA, 22 nt, Homo sapiens
guggguacggcccagugggggg

SEQ ID NO: 812 - RNA, 22 nt, Homo sapiens
ugagccccugugccgcccccag

SEQ ID NO: 813 - RNA, 26 nt, Homo sapiens
gugagggcaugcaggccuggaugggg

SEQ ID NO: 814 - RNA, 22 nt, Homo sapiens
ucaccagcccuguguucccuag

SEQ ID NO: 815 - RNA, 20 nt, Homo sapiens
cgugccacccuuuuccccag

SEQ ID NO: 816 - RNA, 21 nt, Homo sapiens
gugggcgggggcaggugugug

SEQ ID NO: 817 - RNA, 20 nt, Homo sapiens
ucacaccugccucgcccccc

SEQ ID NO: 818 - RNA, 23 nt, Homo sapiens
cucucaccacugcccucccacag

SEQ ID NO: 819 - RNA, 20 nt, Homo sapiens
gugucugggcggacagcugc

SEQ ID NO: 820 - RNA, 20 nt, Homo sapiens
ugagcccuguccucccgcag

SEQ ID NO: 821 - RNA, 22 nt, Homo sapiens
ucggccugaccacccaccccac

SEQ ID NO: 822 - RNA, 22 nt, Homo sapiens
ccucuuccccuugucucuccag

SEQ ID NO: 823 - RNA, 21 nt, Homo sapiens
uccuucugcuccgucccccag

SEQ ID NO: 824 - RNA, 20 nt, Homo sapiens
cuuccucgucugucugcccc

SEQ ID NO: 825 - RNA, 22 nt, Homo sapiens
cuccugagccauucugagccuc

SEQ ID NO: 826 - RNA, 24 nt, Homo sapiens
agccugauuaaacacaugcucuga

SEQ ID NO: 827 - RNA, 21 nt, Homo sapiens
gugccagcugcagugggggag

SEQ ID NO: 828 - RNA, 21 nt, Homo sapiens
cccggagccaggaugcagcuc

SEQ ID NO: 829 - RNA, 22 nt, Homo sapiens
gguggcccggccgugccugagg

SEQ ID NO: 830 - RNA, 21 nt, Homo sapiens
ucguggccuggucuccauuau

SEQ ID NO: 831 - RNA, 20 nt, Homo sapiens
ucugcaggguuugcuuugag

SEQ ID NO: 832 - RNA, 21 nt, Homo sapiens
uguucauguagauguuuaagc

SEQ ID NO: 833 - RNA, 21 nt, Homo sapiens
uggcagggaggcugggagggg

SEQ ID NO: 834 - RNA, 18 nt, Homo sapiens
ucagcuggcccucauuuc

SEQ ID NO: 835 - RNA, 20 nt, Homo sapiens
ucacuguucagacaggcgga

SEQ ID NO: 836 - RNA, 22 nt, Homo sapiens
aaaaacugagacuacuuuugca

SEQ ID NO: 837 - RNA, 22 nt, Homo sapiens
aaaaguaauugcggucuuuggu

SEQ ID NO: 838 - RNA, 22 nt, Homo sapiens
ucugggcaacaaagugagaccu

SEQ ID NO: 839 - RNA, 21 nt, Homo sapiens
ugcaggaccaagaugagcccu

SEQ ID NO: 840 - RNA, 22 nt, Homo sapiens
ugcuggaucagugguucgaguc

SEQ ID NO: 841 - RNA, 23 nt, Homo sapiens
uggaguccaggaaucugcauuuu

SEQ ID NO: 842 - RNA, 19 nt, Homo sapiens
uggauuuuuggaucaggga

SEQ ID NO: 843 - RNA, 24 nt, Homo sapiens
uggcccugacugaagaccagcagu

SEQ ID NO: 844 - RNA, 22 nt, Homo sapiens
aaaaguacuugcggauuuugcu

SEQ ID NO: 845 - RNA, 22 nt, Homo sapiens
uggguggucuggagauuugugc

SEQ ID NO: 846 - RNA, 22 nt, Homo sapiens
ugugagguuggcauuguugucu

SEQ ID NO: 847 - RNA, 21 nt, Homo sapiens
uuaggccgcagaucuggguga

SEQ ID NO: 848 - RNA, 17 nt, Homo sapiens
uucaaguaauucaggug

SEQ ID NO: 849 - RNA, 22 nt, Homo sapiens
uucuggaauucugugugaggga

SEQ ID NO: 850 - RNA, 22 nt, Homo sapiens
aaaaguauuugcggguuuuguc

SEQ ID NO: 851 - RNA, 21 nt, Homo sapiens
uugggacauacuuaugcuaaa

SEQ ID NO: 852 - RNA, 22 nt, Homo sapiens
uuuagagacggggucuugcucu

SEQ ID NO: 853 - RNA, 22 nt, Homo sapiens
uuugaggcuacagugagaugug

SEQ ID NO: 854 - RNA, 22 nt, Homo sapiens
uuuucaacucuaaugggagaga

SEQ ID NO: 855 - RNA, 22 nt, Homo sapiens
aacuggaucaauuauaggagug

SEQ ID NO: 856 - RNA, 19 nt, Homo sapiens
aaaaacuguaauuacuuuu

SEQ ID NO: 857 - RNA, 26 nt, Homo sapiens
aaguaguugguuuguaugagaugguu

SEQ ID NO: 858 - RNA, 21 nt, Homo sapiens
aagugaucuaaaggccuacau

SEQ ID NO: 859 - RNA, 19 nt, Homo sapiens
aauggauuuuuggagcagg

SEQ ID NO: 860 - RNA, 22 nt, Homo sapiens
acccgucccguucguccccgga

SEQ ID NO: 861 - RNA, 27 nt, Homo sapiens
accuucuuguauaagcacugugcuaaa

SEQ ID NO: 862 - RNA, 22 nt, Homo sapiens
acgcccuucccccccuucuuca

SEQ ID NO: 863 - RNA, 21 nt, Homo sapiens
acggugcuggauguggccuuu

SEQ ID NO: 864 - RNA, 21 nt, Homo sapiens
acucuagcugccaaaggcgcu

SEQ ID NO: 865 - RNA, 21 nt, Homo sapiens
agagaagaagaucagccugca

SEQ ID NO: 866 - RNA, 24 nt, Homo sapiens
agccuggaagcuggagccugcagu

SEQ ID NO: 867 - RNA, 23 nt, Homo sapiens
aggaugagcaaagaaaguagauu

SEQ ID NO: 868 - RNA, 22 nt, Homo sapiens
aggcauugacuucucacuagcu

SEQ ID NO: 869 - RNA, 21 nt, Homo sapiens
agugaaugauggguucugacc

SEQ ID NO: 870 - RNA, 21 nt, Homo sapiens
aguuaggauuaggucguggaa

SEQ ID NO: 871 - RNA, 21 nt, Homo sapiens
auauaugaugacuuagcuuuu

SEQ ID NO: 872 - RNA, 18 nt, Homo sapiens
aucccaccucugccacca

SEQ ID NO: 873 - RNA, 22 nt, Homo sapiens
aaaacuguaauuacuuuuguac

SEQ ID NO: 874 - RNA, 19 nt, Homo sapiens
auggauaaggcuuuggcuu

SEQ ID NO: 875 - RNA, 22 nt, Homo sapiens
augggugaauuuguagaaggau

SEQ ID NO: 876 - RNA, 22 nt, Homo sapiens
augguacccuggcauacugagu

SEQ ID NO: 877 - RNA, 22 nt, Homo sapiens
caaaaguaauuguggauuuugu

SEQ ID NO: 878 - RNA, 21 nt, Homo sapiens
caaagguauuugugguuuuug

SEQ ID NO: 879 - RNA, 22 nt, Homo sapiens
caggauguggucaaguguuguu

SEQ ID NO: 880 - RNA, 22 nt, Homo sapiens
ccaaaacugcaguuacuuuugc

SEQ ID NO: 881 - RNA, 23 nt, Homo sapiens
ccucagggcuguagaacagggcu

SEQ ID NO: 882 - RNA, 21 nt, Homo sapiens
ccuguugaaguguaaucccca

SEQ ID NO: 883 - RNA, 18 nt, Homo sapiens
cgggcguggugguggggg

SEQ ID NO: 884 - RNA, 22 nt, Homo sapiens
cuggacugagccgugcuacugg

SEQ ID NO: 885 - RNA, 23 nt, Homo sapiens
cuggagauauggaagagcugugu

SEQ ID NO: 886 - RNA, 26 nt, Homo sapiens
gaugaugauggcagcaaauucugaaa

SEQ ID NO: 887 - RNA, 25 nt, Homo sapiens
gggcgacaaagcaagacucuuucuu

SEQ ID NO: 888 - RNA, 18 nt, Homo sapiens
gucccuguucaggcgcca

SEQ ID NO: 889 - RNA, 22 nt, Homo sapiens
aaaaguaaucgcgguuuuuguc

SEQ ID NO: 890 - RNA, 17 nt, Homo sapiens
gugggggagaggcuguc

SEQ ID NO: 891 - RNA, 20 nt, Homo sapiens
uaaagagcccuguggagaca

SEQ ID NO: 892 - RNA, 17 nt, Homo sapiens
uaagugcuuccaugcuu

SEQ ID NO: 893 - RNA, 17 nt, Homo sapiens
uaauugcuuccauguuu

SEQ ID NO: 894 - RNA, 22 nt, Homo sapiens
uacguagauauauauguauuuu

SEQ ID NO: 895 - RNA, 22 nt, Homo sapiens
uagcaaaaacugcaguuacuuu

SEQ ID NO: 896 - RNA, 22 nt, Homo sapiens
aaaaguaauugcggauuuugcc

SEQ ID NO: 897 - RNA, 22 nt, Homo sapiens
uaguacugugcauaucaucuau

SEQ ID NO: 898 - RNA, 17 nt, Homo sapiens
ucauauugcuucuuucu

SEQ ID NO: 899 - RNA, 17 nt, Homo sapiens
ucccuguucgggcgcca

SEQ ID NO: 900 - RNA, 17 nt, Homo sapiens
ucgccuccuccucuccc

SEQ ID NO: 901 - RNA, 20 nt, Homo sapiens
ucguuugccuuuuucugcuu

SEQ ID NO: 902 - RNA, 22 nt, Homo sapiens
ucuauacagacccuggcuuuuc

SEQ ID NO: 903 - RNA, 21 nt, Homo sapiens
uggacugcccugaucuggaga

SEQ ID NO: 904 - RNA, 25 nt, Homo sapiens
ugggaacggguuccggcagacgcug

SEQ ID NO: 905 - RNA, 22 nt, Homo sapiens
agaaggaaauugaauucauuua

SEQ ID NO: 906 - RNA, 22 nt, Homo sapiens
cggaugagcaaagaaagugguu

SEQ ID NO: 907 - RNA, 17 nt, Homo sapiens
ucccaccgcugccaccc

SEQ ID NO: 908 - RNA, 18 nt, Homo sapiens
gcaugggugguucagugg

SEQ ID NO: 909 - RNA, 24 nt, Homo sapiens
acuggcuagggaaaaugauuggau

SEQ ID NO: 910 - RNA, 23 nt, Homo sapiens
uauucauuuauccccagccuaca

SEQ ID NO: 911 - RNA, 18 nt, Homo sapiens
acguuggcucugguggug

SEQ ID NO: 912 - RNA, 22 nt, Homo sapiens
acucggcguggcgucggucgug

SEQ ID NO: 913 - RNA, 22 nt, Homo sapiens
uucacaaggaggugucauuuau

SEQ ID NO: 914 - RNA, 22 nt, Homo sapiens
uucucaaggaggugucguuuau

SEQ ID NO: 915 - RNA, 18 nt, Homo sapiens
cagggaggugaaugugau

SEQ ID NO: 916 - RNA, 19 nt, Homo sapiens
gaugaugcugcugaugcug

SEQ ID NO: 917 - RNA, 17 nt, Homo sapiens
ucucgcuggggccucca

SEQ ID NO: 918 - RNA, 21 nt, Homo sapiens
uaggacacauggucuacuucu

SEQ ID NO: 919 - RNA, 24 nt, Homo sapiens
ccagacagaauucuaugcacuuuc

SEQ ID NO: 920 - RNA, 22 nt, Homo sapiens
cucggcgcggggcgcgggcucc

SEQ ID NO: 921 - RNA, 21 nt, Homo sapiens
gcccuccgcccgugcaccccg

SEQ ID NO: 922 - RNA, 22 nt, Homo sapiens
gcccgcguguggagccaggugu

SEQ ID NO: 923 - RNA, 22 nt, Homo sapiens
aaaaccgucuaguuacaguugu

SEQ ID NO: 924 - RNA, 23 nt, Homo sapiens
cggcccgggcugcugcuguuccu

SEQ ID NO: 925 - RNA, 21 nt, Homo sapiens
uccugcgcgucccagaugccc

SEQ ID NO: 926 - RNA, 23 nt, Homo sapiens
ucauagcccuguacaaugcugcu

SEQ ID NO: 927 - RNA, 19 nt, Homo sapiens
aaaagcuggguugagagga

SEQ ID NO: 928 - RNA, 18 nt, Homo sapiens
uccagugcccuccucucc

SEQ ID NO: 929 - RNA, 27 nt, Homo sapiens
auugaucaucgacacuucgaacgcaau

SEQ ID NO: 930 - RNA, 18 nt, Homo sapiens
ugaggcaguagauugaau

SEQ ID NO: 931 - RNA, 21 nt, Homo sapiens
cggcggggacggcgauugguc

SEQ ID NO: 932 - RNA, 21 nt, Homo sapiens
ugagugccggugccugcccug

SEQ ID NO: 933 - RNA, 22 nt, Homo sapiens
cgcaggggccgggugcucaccg

SEQ ID NO: 934 - RNA, 21 nt, Homo sapiens
ccaguccugugccugccgccu

SEQ ID NO: 935 - RNA, 23 nt, Homo sapiens
ugaguaccgccaugucuguuggg

SEQ ID NO: 936 - RNA, 20 nt, Homo sapiens
caccaggcauuguggucucc

SEQ ID NO: 937 - RNA, 22 nt, Homo sapiens
uacccagagcaugcagugugaa

SEQ ID NO: 938 - RNA, 22 nt, Homo sapiens
ucugcccccuccgcugcugcca

SEQ ID NO: 939 - RNA, 22 nt, Homo sapiens
cccugugcccggcccacuucug

SEQ ID NO: 940 - RNA, 22 nt, Homo sapiens
ggaggggucccgcacugggagg

SEQ ID NO: 941 - RNA, 22 nt, Homo sapiens
accuugccuugcugcccgggcc

SEQ ID NO: 942 - RNA, 20 nt, Homo sapiens
ccccagggcgacgcggcggg

SEQ ID NO: 943 - RNA, 22 nt, Homo sapiens
ucaggccaggcacaguggcuca

SEQ ID NO: 944 - RNA, 19 nt, Homo sapiens
accgugcaaagguagcaua

SEQ ID NO: 945 - RNA, 25 nt, Homo sapiens
cccccacaaccgcgcuugacuagcu

SEQ ID NO: 946 - RNA, 20 nt, Homo sapiens
ccuccugcccuccuugcugu

SEQ ID NO: 947 - RNA, 22 nt, Homo sapiens
cucccacugcuucacuugacua

SEQ ID NO: 948 - RNA, 20 nt, Homo sapiens
uguuuugauaacaguaaugu

SEQ ID NO: 949 - RNA, 23 nt, Homo sapiens
guguuaauuaaaccucuauuuac

SEQ ID NO: 950 - RNA, 23 nt, Homo sapiens
cuguaauauaaauuuaauuuauu

SEQ ID NO: 951 - RNA, 22 nt, Homo sapiens
uuggggaaacggccgcugagug

SEQ ID NO: 952 - RNA, 22 nt, Homo sapiens
uagucccuuccuugaagcgguc

SEQ ID NO: 953 - RNA, 21 nt, Homo sapiens
cgagccucaagcaagggacuu

SEQ ID NO: 954 - RNA, 22 nt, Homo sapiens
agcuuccaugacuccugaugga

SEQ ID NO: 955 - RNA, 22 nt, Homo sapiens
caucagaauucauggaggcuag

SEQ ID NO: 956 - RNA, 21 nt, Homo sapiens
gguucuuagcauaggaggucu

SEQ ID NO: 957 - RNA, 21 nt, Homo sapiens
ccucccaugccaagaacuccc

SEQ ID NO: 958 - RNA, 21 nt, Homo sapiens
uguucucuuugccaaggacag

SEQ ID NO: 959 - RNA, 22 nt, Homo sapiens
gcuggugcaaaaguaauggcgg

SEQ ID NO: 960 - RNA, 22 nt, Homo sapiens
ucugcaagugucagaggcgagg

SEQ ID NO: 961 - RNA, 21 nt, Homo sapiens
ugacagcgcccugccuggcuc

SEQ ID NO: 962 - RNA, 22 nt, Homo sapiens
gagagcaguguguguugccugg

SEQ ID NO: 963 - RNA, 22 nt, Homo sapiens
gggacccagggagagacguaag

SEQ ID NO: 964 - RNA, 21 nt, Homo sapiens
cuuccgccccgccgggcgucg

SEQ ID NO: 965 - RNA, 19 nt, Homo sapiens
ggggccuggcggugggcgg

SEQ ID NO: 966 - RNA, 21 nt, Homo sapiens
guuagggccaacaucucuugg

SEQ ID NO: 967 - RNA, 20 nt, Homo sapiens
auauggguuuacuaguuggu

SEQ ID NO: 968 - RNA, 22 nt, Homo sapiens
ugccuggaacauaguagggacu

SEQ ID NO: 969 - RNA, 21 nt, Homo sapiens
auaggacucauauagugccag

SEQ ID NO: 970 - RNA, 23 nt, Homo sapiens
ugugacugcauuaugaaaauucu

SEQ ID NO: 971 - RNA, 20 nt, Homo sapiens
uggcuuuuaacuuugauggc

SEQ ID NO: 972 - RNA, 21 nt, Homo sapiens
cacagcaaguguagacaggca

SEQ ID NO: 973 - RNA, 22 nt, Homo sapiens
uaaauagaguaggcaaaggaca

SEQ ID NO: 974 - RNA, 22 nt, Homo sapiens
guugggacaagaggacggucuu

SEQ ID NO: 975 - RNA, 17 nt, Homo sapiens
cagagaauuguuuaauc

SEQ ID NO: 976 - RNA, 21 nt, Homo sapiens
uucgcgggcgaaggcaaaguc

SEQ ID NO: 977 - RNA, 23 nt, Homo sapiens
auggccaaaacugcaguuauuuu

SEQ ID NO: 978 - RNA, 20 nt, Homo sapiens
uagaggaagcuguggagaga

SEQ ID NO: 979 - RNA, 22 nt, Homo sapiens
ugagggacagaugccagaagca

SEQ ID NO: 980 - RNA, 22 nt, Homo sapiens
caucuggcauccgucacacaga

SEQ ID NO: 981 - RNA, 23 nt, Homo sapiens
aucagggcuuguggaaugggaag

SEQ ID NO: 982 - RNA, 23 nt, Homo sapiens
ucuggcaaguaaaaaacucucau

SEQ ID NO: 983 - RNA, 22 nt, Homo sapiens
gcaguaguguagagauugguuu

SEQ ID NO: 984 - RNA, 21 nt, Homo sapiens
uacccagucuccggugcagcc

SEQ ID NO: 985 - RNA, 21 nt, Homo sapiens
gcugcaccggagacuggguaa

SEQ ID NO: 986 - RNA, 23 nt, Homo sapiens
ucgaggacugguggaagggccuu

SEQ ID NO: 987 - RNA, 24 nt, Homo sapiens
uggguagagaaggagcucagagga

SEQ ID NO: 988 - RNA, 22 nt, Homo sapiens
uaaagaacucuuaaaacccaau

SEQ ID NO: 989 - RNA, 19 nt, Homo sapiens
acuggacuuggaggcagaa

SEQ ID NO: 990 - RNA, 23 nt, Homo sapiens
ugauggauaaaagacuacauauu

SEQ ID NO: 991 - RNA, 22 nt, Homo sapiens
ugccuaggcugagacugcagug

SEQ ID NO: 992 - RNA, 23 nt, Homo sapiens
auacacauacacgcaacacacau

SEQ ID NO: 993 - RNA, 23 nt, Homo sapiens
cugacugaauagguagggucauu

SEQ ID NO: 994 - RNA, 22 nt, Homo sapiens
accugagguugugcauuucuaa

SEQ ID NO: 995 - RNA, 24 nt, Homo sapiens
ucuguagccugggagcaauggggu

SEQ ID NO: 996 - RNA, 24 nt, Homo sapiens
uguggacagugagguagagggagu

SEQ ID NO: 997 - RNA, 23 nt, Homo sapiens
uaggagcucaacagaugccuguu

SEQ ID NO: 998 - RNA, 22 nt, Homo sapiens
agcuuuugggaauucagguagu

SEQ ID NO: 999 - RNA, 21 nt, Homo sapiens
caaaagugaucgugguuuuug

SEQ ID NO: 1000 - RNA, 19 nt, Homo sapiens
gagggcggguggaggagga

SEQ ID NO: 1001 - RNA, 22 nt, Homo sapiens
aaggccuuucugaaccuucaga

SEQ ID NO: 1002 - RNA, 25 nt, Homo sapiens
auaacauuguaaagcgcuucuuucg

SEQ ID NO: 1003 - RNA, 23 nt, Homo sapiens
caaagacugcaauuacuuuugcg

SEQ ID NO: 1004 - RNA, 22 nt, Homo sapiens
aggggaccaaagagauauauag

SEQ ID NO: 1005 - RNA, 22 nt, Homo sapiens
auauaccuguucggucucuuua

SEQ ID NO: 1006 - RNA, 24 nt, Homo sapiens
agauauuuugaguguuuggaauug

SEQ ID NO: 1007 - RNA, 22 nt, Homo sapiens
ggcgacaaaacgagacccuguc

SEQ ID NO: 1008 - RNA, 22 nt, Homo sapiens
caugcuaggauagaaagaaugg

SEQ ID NO: 1009 - RNA, 24 nt, Homo sapiens
gguugggcagugaggaggguguga

SEQ ID NO: 1010 - RNA, 22 nt, Homo sapiens
agcuacaguuacuuuugcacca

SEQ ID NO: 1011 - RNA, 22 nt, Homo sapiens
uggaaaaaacuggugugugcuu

SEQ ID NO: 1012 - RNA, 23 nt, Homo sapiens
uuuguauggauauguguguguau

SEQ ID NO: 1013 - RNA, 22 nt, Homo sapiens
cuggggagauccucgagguugg

SEQ ID NO: 1014 - RNA, 21 nt, Homo sapiens
gguggggcaaugggaucaggu

SEQ ID NO: 1015 - RNA, 22 nt, Homo sapiens
uguguuagaauaggggcaauaa

SEQ ID NO: 1016 - RNA, 23 nt, Homo sapiens
ggggaaagcgaguagggacauuu

SEQ ID NO: 1017 - RNA, 22 nt, Homo sapiens
gauaucagcucaguaggcaccg

SEQ ID NO: 1018 - RNA, 22 nt, Homo sapiens
cagaaggggaguugggagcaga

SEQ ID NO: 1019 - RNA, 21 nt, Homo sapiens
ccaggcucugcagugggaacu

SEQ ID NO: 1020 - RNA, 22 nt, Homo sapiens
aaagaucuggaagugggagaca

SEQ ID NO: 1021 - RNA, 22 nt, Homo sapiens
uucagccaggcuagugcagucu

SEQ ID NO: 1022 - RNA, 22 nt, Homo sapiens
aagggcuuccucucugcaggac

SEQ ID NO: 1023 - RNA, 22 nt, Homo sapiens
uaggauuacaagugucggccac

SEQ ID NO: 1024 - RNA, 22 nt, Homo sapiens
agagcugagacuagaaagccca

SEQ ID NO: 1025 - RNA, 23 nt, Homo sapiens
cugauaagaacagaggcccagau

SEQ ID NO: 1026 - RNA, 23 nt, Homo sapiens
uuagggaguagaaggguggggag

SEQ ID NO: 1027 - RNA, 22 nt, Homo sapiens
uauaaaaugagggcaguaagac

SEQ ID NO: 1028 - RNA, 22 nt, Homo sapiens
ugugacuuuaagggaaauggcg

SEQ ID NO: 1029 - RNA, 22 nt, Homo sapiens
agguggaugcaaugugaccuca

SEQ ID NO: 1030 - RNA, 23 nt, Homo sapiens
cgcagacaaugccuacuggccua

SEQ ID NO: 1031 - RNA, 19 nt, Homo sapiens
aucccaccacugccaccau

SEQ ID NO: 1032 - RNA, 22 nt, Homo sapiens
aggauuucagaaauacuggugu

SEQ ID NO: 1033 - RNA, 17 nt, Homo sapiens
gaguucuacagucagac

SEQ ID NO: 1034 - RNA, 22 nt, Homo sapiens
uaggacugugcuuggcacauag

SEQ ID NO: 1035 - RNA, 22 nt, Homo sapiens
cugggguucugagacagacagu

SEQ ID NO: 1036 - RNA, 24 nt, Homo sapiens
agauguauggaaucuguauauauc

SEQ ID NO: 1037 - RNA, 19 nt, Homo sapiens
ugggguuuugcaguccuua

SEQ ID NO: 1038 - RNA, 22 nt, Homo sapiens
aaaggaggaaauaggcaggcca

SEQ ID NO: 1039 - RNA, 23 nt, Homo sapiens
gggaugguagaccggugacgugc

SEQ ID NO: 1040 - RNA, 23 nt, Homo sapiens
agguuguccguggugaguucgca

SEQ ID NO: 1041 - RNA, 22 nt, Homo sapiens
cccaauacacggucgaccucuu

SEQ ID NO: 1042 - RNA, 23 nt, Homo sapiens
uagugaguuagagaugcagagcc

SEQ ID NO: 1043 - RNA, 22 nt, Homo sapiens
cggggagagaacgcagugacgu

SEQ ID NO: 1044 - RNA, 19 nt, Homo sapiens
acuggccugggacuaccgg

SEQ ID NO: 1045 - RNA, 21 nt, Homo sapiens
ugcacggcacuggggacacgu

SEQ ID NO: 1046 - RNA, 17 nt, Homo sapiens
ggggcgcggccggaucg

SEQ ID NO: 1047 - RNA, 22 nt, Homo sapiens
agaaggggugaaauuuaaacgu

SEQ ID NO: 1048 - RNA, 25 nt, Homo sapiens
cuuccagacgcuccgccccacgucg

SEQ ID NO: 1049 - RNA, 22 nt, Homo sapiens
uggggcggagcuuccggaggcc

SEQ ID NO: 1050 - RNA, 23 nt, Homo sapiens
aaaaguaacugcgguuuuugccu

SEQ ID NO: 1051 - RNA, 19 nt, Homo sapiens
aucgggcccucggcgccgg

SEQ ID NO: 1052 - RNA, 17 nt, Homo sapiens
gcuucuguaguguaguc

SEQ ID NO: 1053 - RNA, 22 nt, Homo sapiens
gccucucucggagucgcucgga

SEQ ID NO: 1054 - RNA, 24 nt, Homo sapiens
ugaggggccucagaccgagcuuuu

SEQ ID NO: 1055 - RNA, 23 nt, Homo sapiens
agaagaaggcggucggucugcgg

SEQ ID NO: 1056 - RNA, 23 nt, Homo sapiens
ucaacaaaaucacugaugcugga

SEQ ID NO: 1057 - RNA, 23 nt, Homo sapiens
ucagcaccaggauauuguuggag

SEQ ID NO: 1058 - RNA, 22 nt, Homo sapiens
caggcgucugucuacguggcuu

SEQ ID NO: 1059 - RNA, 21 nt, Homo sapiens
ucacgcggagagauggcuuug

SEQ ID NO: 1060 - RNA, 20 nt, Homo sapiens
uuggccauggggcugcgcgg

SEQ ID NO: 1061 - RNA, 23 nt, Homo sapiens
agaggcuuugugcggauacgggg

SEQ ID NO: 1062 - RNA, 21 nt, Homo sapiens
cccuugggucugaugggguag

SEQ ID NO: 1063 - RNA, 18 nt, Homo sapiens
aaagcuggguugagaagg

SEQ ID NO: 1064 - RNA, 23 nt, Homo sapiens
uguggaagguagacggccagaga

SEQ ID NO: 1065 - RNA, 22 nt, Homo sapiens
uggaagguagacggccagagag

SEQ ID NO: 1066 - RNA, 23 nt, Homo sapiens
uggggacguagcuggccagacag

SEQ ID NO: 1067 - RNA, 23 nt, Homo sapiens
ucugggagguuguagcaguggaa

SEQ ID NO: 1068 - RNA, 22 nt, Homo sapiens
uccugcguaggaucugaggagu

SEQ ID NO: 1069 - RNA, 21 nt, Homo sapiens
ggccagccaccaggagggcug

SEQ ID NO: 1070 - RNA, 17 nt, Homo sapiens
cgcgccgggcccggguu

SEQ ID NO: 1071 - RNA, 18 nt, Homo sapiens
cggggcggcaggggccuc

SEQ ID NO: 1072 - RNA, 21 nt, Homo sapiens
uaaaaacugcaauuacuuuca

SEQ ID NO: 1073 - RNA, 23 nt, Homo sapiens
ggaggcgcaggcucggaaaggcg

SEQ ID NO: 1074 - RNA, 22 nt, Homo sapiens
guggaguccuggggaauggaga

SEQ ID NO: 1075 - RNA, 23 nt, Homo sapiens
agggacugccuuaggagaaaguu

SEQ ID NO: 1076 - RNA, 22 nt, Homo sapiens
caccuugcgcuacucaggucug

SEQ ID NO: 1077 - RNA, 17 nt, Homo sapiens
gggauaugaagaaaaau

SEQ ID NO: 1078 - RNA, 22 nt, Homo sapiens
uucucaagagggaggcaaucau

SEQ ID NO: 1079 - RNA, 21 nt, Homo sapiens
auugacaccucugugagugga

SEQ ID NO: 1080 - RNA, 22 nt, Homo sapiens
uggaagggagaagagcuuuaau

SEQ ID NO: 1081 - RNA, 25 nt, Homo sapiens
gaacccaugagguugaggcugcagu

SEQ ID NO: 1082 - RNA, 18 nt, Homo sapiens
cagugcaauguuuuccuu

SEQ ID NO: 1083 - RNA, 17 nt, Homo sapiens
augugggcucaggcuca

SEQ ID NO: 1084 - RNA, 16 nt, Homo sapiens
ugccuuccugucugug

SEQ ID NO: 1085 - RNA, 25 nt, Homo sapiens
acuggacuuggagucagaagagugg

SEQ ID NO: 1086 - RNA, 17 nt, Homo sapiens
cagccugacaggaacag

SEQ ID NO: 1087 - RNA, 17 nt, Homo sapiens
gggagucuacagcaggg

SEQ ID NO: 1088 - RNA, 20 nt, Homo sapiens
ucccacuacuucacuuguga

SEQ ID NO: 1089 - RNA, 18 nt, Homo sapiens
gcuggugacaugagaggc

SEQ ID NO: 1090 - RNA, 22 nt, Homo sapiens
cugggacaggaggaggaggcag

SEQ ID NO: 1091 - RNA, 18 nt, Homo sapiens
ugggagcuggacuacuuc

SEQ ID NO: 1092 - RNA, 17 nt, Homo sapiens
ccggcauguccagggca

SEQ ID NO: 1093 - RNA, 18 nt, Homo sapiens
ccaguguggcucagcgag

SEQ ID NO: 1094 - RNA, 17 nt, Homo sapiens
uucugagcugaggacag

SEQ ID NO: 1095 - RNA, 18 nt, Homo sapiens
ccuagacaccuccaguuc

SEQ ID NO: 1096 - RNA, 17 nt, Homo sapiens
uggagagaaaggcagua

SEQ ID NO: 1097 - RNA, 18 nt, Homo sapiens
cuggagucuaggauucca

SEQ ID NO: 1098 - RNA, 19 nt, Homo sapiens
aauguuuuuuccuguuucc

SEQ ID NO: 1099 - RNA, 18 nt, Homo sapiens
ucccuggaguuucuucuu

SEQ ID NO: 1100 - RNA, 16 nt, Homo sapiens
gcagcauucauguccc

SEQ ID NO: 1101 - RNA, 18 nt, Homo sapiens
gaaagagagcugagugug

SEQ ID NO: 1102 - RNA, 19 nt, Homo sapiens
ggccuuguuccugucccca

SEQ ID NO: 1103 - RNA, 20 nt, Homo sapiens
agcccccuggccccaaaccc

SEQ ID NO: 1104 - RNA, 18 nt, Homo sapiens
ccgcuuucugagcuggac

SEQ ID NO: 1105 - RNA, 17 nt, Homo sapiens
ggugaggcuagcuggug

SEQ ID NO: 1106 - RNA, 18 nt, Homo sapiens
cucugggaaaugggacag

SEQ ID NO: 1107 - RNA, 17 nt, Homo sapiens
cacuguggguacaugcu

SEQ ID NO: 1108 - RNA, 17 nt, Homo sapiens
ucccugagcaaagccac

SEQ ID NO: 1109 - RNA, 18 nt, Homo sapiens
gggauucuguagcuuccu

SEQ ID NO: 1110 - RNA, 17 nt, Homo sapiens
acauugccagggaguuu

SEQ ID NO: 1111 - RNA, 21 nt, Homo sapiens
cugugggcucagcgcgugggg

SEQ ID NO: 1112 - RNA, 21 nt, Homo sapiens
uuagcgguggaccgcccugcg

SEQ ID NO: 1113 - RNA, 18 nt, Homo sapiens
cagccccacagccucaga

SEQ ID NO: 1114 - RNA, 20 nt, Homo sapiens
cccugagacccuaaccuuaa

SEQ ID NO: 1115 - RNA, 18 nt, Homo sapiens
aucugaccugaugaaggu

SEQ ID NO: 1116 - RNA, 18 nt, Homo sapiens
ccagagguggggacugag

SEQ ID NO: 1117 - RNA, 17 nt, Homo sapiens
ccccgccaccgccuugg

SEQ ID NO: 1118 - RNA, 22 nt, Homo sapiens
caguugggucuaggggucagga

SEQ ID NO: 1119 - RNA, 19 nt, Homo sapiens
cuuggggcauggaguccca

SEQ ID NO: 1120 - RNA, 18 nt, Homo sapiens
agggcauguccagggggu

SEQ ID NO: 1121 - RNA, 17 nt, Homo sapiens
ccugagaaaagggccaa

SEQ ID NO: 1122 - RNA, 23 nt, Homo sapiens
gccuggagcuacuccaccaucuc

SEQ ID NO: 1123 - RNA, 17 nt, Homo sapiens
caguguucagagaugga

SEQ ID NO: 1124 - RNA, 19 nt, Homo sapiens
ggccacugagucagcacca

SEQ ID NO: 1125 - RNA, 18 nt, Homo sapiens
uugcacuugucucaguga

SEQ ID NO: 1126 - RNA, 20 nt, Homo sapiens
uguuccucugucucccagac

SEQ ID NO: 1127 - RNA, 19 nt, Homo sapiens
ggcuugcaugggggacugg

SEQ ID NO: 1128 - RNA, 16 nt, Homo sapiens
aggaaacagggaccca

SEQ ID NO: 1129 - RNA, 19 nt, Homo sapiens
cugugggcucagcucuggg

SEQ ID NO: 1130 - RNA, 16 nt, Homo sapiens
cuaggaggccuuggcc

SEQ ID NO: 1131 - RNA, 16 nt, Homo sapiens
uccagcucgguggcac

SEQ ID NO: 1132 - RNA, 17 nt, Homo sapiens
gacauucagacuaccug

SEQ ID NO: 1133 - RNA, 21 nt, Homo sapiens
auccccagauacaauggacaa

SEQ ID NO: 1134 - RNA, 21 nt, Homo sapiens
ggcuccuccucucaggaugug

SEQ ID NO: 1135 - RNA, 21 nt, Homo sapiens
gcaggcacagacagcccuggc

SEQ ID NO: 1136 - RNA, 18 nt, Homo sapiens
auucuaagugccuuggcc

SEQ ID NO: 1137 - RNA, 17 nt, Homo sapiens
acucagucauggucauu

SEQ ID NO: 1138 - RNA, 20 nt, Homo sapiens
ucagggagucaggggagggc

SEQ ID NO: 1139 - RNA, 19 nt, Homo sapiens
gggggaagaaaaggugggg

SEQ ID NO: 1140 - RNA, 18 nt, Homo sapiens
cauucaacuagugauugu

SEQ ID NO: 1141 - RNA, 18 nt, Homo sapiens
guguucucugauggacag

SEQ ID NO: 1142 - RNA, 17 nt, Homo sapiens
cucagugacucaugugc

SEQ ID NO: 1143 - RNA, 17 nt, Homo sapiens
ccaauuaccacuucuuu

SEQ ID NO: 1144 - RNA, 18 nt, Homo sapiens
cagcagucccucccccug

SEQ ID NO: 1145 - RNA, 18 nt, Homo sapiens
gggucccggggagggggg

SEQ ID NO: 1146 - RNA, 21 nt, Homo sapiens
gcaguucugagcacaguacac

SEQ ID NO: 1147 - RNA, 16 nt, Homo sapiens
cucuccucccggcuuc

SEQ ID NO: 1148 - RNA, 18 nt, Homo sapiens
cuaggggguuugcccuug

SEQ ID NO: 1149 - RNA, 21 nt, Homo sapiens
gaguguaguucugagcagagc

SEQ ID NO: 1150 - RNA, 18 nt, Homo sapiens
uaaaauuugcauccagga

SEQ ID NO: 1151 - RNA, 18 nt, Homo sapiens
gcggcgaguccgacucau

SEQ ID NO: 1152 - RNA, 17 nt, Homo sapiens
uggggcucagcgaguuu

SEQ ID NO: 1153 - RNA, 18 nt, Homo sapiens
gggcucacaucaccccau

SEQ ID NO: 1154 - RNA, 17 nt, Homo sapiens
accccacuccugguacc

SEQ ID NO: 1155 - RNA, 19 nt, Homo sapiens
ucucccuugagggcacuuu

SEQ ID NO: 1156 - RNA, 17 nt, Homo sapiens
uugucugcugaguuucc

SEQ ID NO: 1157 - RNA, 18 nt, Homo sapiens
ccccugggccggccuugg

SEQ ID NO: 1158 - RNA, 19 nt, Homo sapiens
gcauugugcagggcuauca

SEQ ID NO: 1159 - RNA, 19 nt, Homo sapiens
ugcccuccuuucuucccuc

SEQ ID NO: 1160 - RNA, 16 nt, Homo sapiens
uucagcaggaacagcu

SEQ ID NO: 1161 - RNA, 19 nt, Homo sapiens
ccugagacccuaguuccac

SEQ ID NO: 1162 - RNA, 19 nt, Homo sapiens
ccucagaucagagccuugc

SEQ ID NO: 1163 - RNA, 18 nt, Homo sapiens
aauccuugcuaccugggu

SEQ ID NO: 1164 - RNA, 17 nt, Homo sapiens
ccaguuuucccaggauu